(19) United States
(12) Patent Application Publication
(10) Pub. No.: US 2026/0285878 A1
(43) Pub. Date: Sep. 24, 2026

(54) SMALL MOLECULE GLP-1 AGONISTS

(71) Applicant: Ambrosia Biosciences Inc., Boulder, CO (US)

(72) Inventors: Donald Backos, Aurora, CO (US); Bismarck Campos, Dacono, CO (US); Natalie Dwulet, Boulder, CO (US); Erik James Hicken, Longmont, CO (US); Michael Hilton, Broomfield, CO (US); Dean Kahn, Longmont, CO (US); Robert Pipal, Boulder, CO (US)

(21) Appl. No.: 19/462,025

(22) Filed: Jan. 28, 2026

Related U.S. Application Data
(60) Provisional application No. 63/750,572, filed on Jan. 28, 2025.
(60) Provisional application No. 63/750,578, filed on Jan. 28, 2025.
(60) Provisional application No. 63/858,669, filed on Aug. 6, 2025.

(51) Int. Cl. C07D 498/22; A61K 31/439; A61K 31/444; A61K 31/501; A61K 31/506; A61K 31/5377; A61K 31/5383; A61K 31/5386; A61K 31/55; A61K 31/553; C07D 471/22; C07D 513/22; C07D 519/00

(52) U.S. Cl. CPC C07D 498/22; A61K 31/439; A61K 31/444; A61K 31/501; A61K 31/506; A61K 31/5377; A61K 31/5383; A61K 31/5386; A61K 31/55; A61K 31/553; C07D 471/22; C07D 513/22; C07D 519/00

(57) ABSTRACT

Disclosed are compounds of formulae (I) and (I-a), and pharmaceutically acceptable salts thereof, which are useful as GLP-1R agonists. Also provided are pharmaceutical compositions comprising a compound of formula (I) or (I-a), or a pharmaceutically acceptable salt thereof, and methods involving use of the compounds or pharmaceutically acceptable salts thereof and compositions in the treatment of various GLP-1-associated diseases, disorders, and conditions, such as obesity.

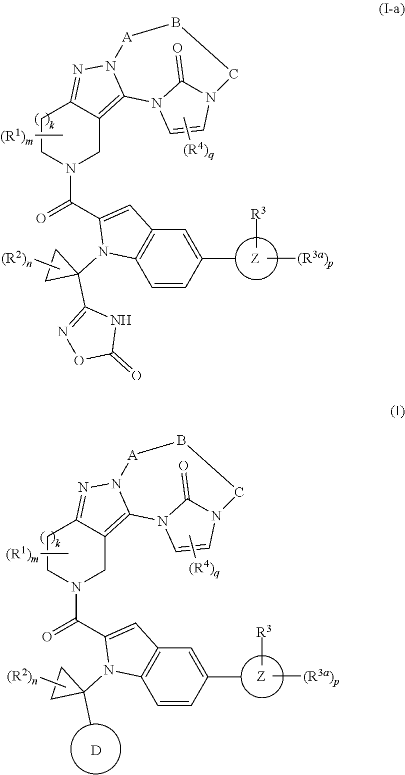

### CROSS REFERENCE TO RELATED APPLICATIONS

This application claims the benefit of priority to U.S. Provisional Patent Application Nos. 63/858,669, filed Aug. 6, 2025; 63/750,578, filed Jan. 28, 2025; and 63/750,572, filed Jan. 28, 2025.

### BACKGROUND

Glucagon-like peptide-1 (GLP-1) is an incretin metabolic hormone which plays a critical role in the regulation of glucose homeostasis. When food passes through the digestive tract, GLP-1 is secreted into the bloodstream by L cells of the small intestine. Binding of GLP-1 to glucagon-like peptide-1 receptors (GLP-1R) in the pancreas causes an increase in insulation secretion and a decrease in glucagon secretion, ultimately resulting in a reduction in blood sugar levels. GLP-1 production causes a variety of other physiological effects, such as decreasing gastric motility and increasing satiety. Synthetic GLP-1R agonists are effective therapeutic agents for the treatment of both diabetes and obesity because of their ability to decrease blood sugar levels and appetite. As GLP-1 has been shown to exert neuroprotective effects, GLP-1R agonists may also be used to treat neurodegenerative conditions such as Huntington's disease. While GLP-1R agonists have demonstrated therapeutic value, the GLP-1R agonists developed to date have poor oral bioavailability and require invasive subcutaneous administration.

Thus, there remains a need for additional GLP-1R agonists which can be non-invasively administered and which will be suitable for various important therapeutic applications.

### SUMMARY

In certain aspects, provided herein are compounds of formula (I):

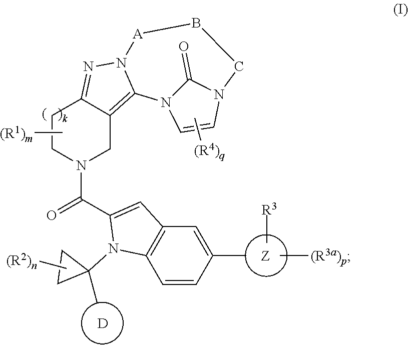

or pharmaceutically acceptable salts thereof; wherein,
A is optionally substituted arylene, heteroarylene, or heterocycloalkylene;
B is -(alkylene)O(alkylene)-, alkylene, haloalkylene, alkenylene, —O(alkylene)-, -(alkylene)O—, —O(alkylene)O—, —O(alkylene)O(alkylene)-, -(alkylene)O(alkylene)O—, —S(alkylene)-, -(alkylene)S—, -(alkylene)S(alkylene)-, —S(alkylene)S—, —S(alkylene)S(alkylene)-, -(alkylene)S(alkylene)S—, —NH(alkylene)-, -(alkylene)NH—, -(alkylene)NH(alkylene)-, —NH(alkylene)NH—, —NH(alkylene)NH(alkylene)-, -(alkylene)NH(alkylene)NH—, —N(alkyl)(alkylene)-, -(alkylene)N(alkyl)-, or is a bond;
C is optionally substituted arylene, heteroarylene, or heterocycloalkylene;
Ring D is

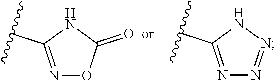

Ring Z is heterocycloalkylene or heteroarylene;
R¹ is independently for each occurrence alkyl, alkoxy, or halo; or two geminal occurrences of R¹ taken together with the carbon atom to which they are attached form an optionally substituted spirocyclic cycloalkyl ring;
R² is independently for each occurrence alkyl, alkoxy, or halo;
R³ is haloalkyl, unsubstituted cycloalkyl, cycloalkyl substituted with alkyl, cycloalkyl substituted with haloalkyl, cycloalkyl substituted with halo, unsubstituted heteroaryl, heteroaryl substituted with alkyl, heteroaryl substituted with halo, unsubstituted heterocycloalkyl, heterocycloalkyl substituted with alkyl, heterocycloalkyl substituted with halo, haloalkoxy, —NH₂, —NH(alkyl), or —N(alkyl)₂;
R^{3a} is independently for each occurrence alkyl, alkoxy, halo, haloalkyl, optionally substituted cycloalkyl, optionally substituted heteroaryl, optionally substituted heterocycloalkyl, haloalkoxy, —NH₂, —NH(alkyl), or —N(alkyl)₂;
R⁴ is independently for each occurrence alkyl, alkoxy, or halo;
k is an integer selected from 0-2;
m is an integer selected from 0-6;
n is an integer selected from 0-4;
p is an integer selected from 0-4; and
q is an integer selected from 0-2.

In certain aspects, provided herein are pharmaceutical compositions comprising a compound of the disclosure, or a pharmaceutically acceptable salt thereof, and a pharmaceutically acceptable excipient.

In certain aspects, provided herein are methods of inducing GLP-1R activity in a subject in need thereof, the methods comprising administering to the subject a therapeutically effective amount of a compound of the disclosure, or a pharmaceutically acceptable salt thereof, or a pharmaceutical composition of the disclosure.

In certain aspects, provided herein are methods of treating GLP-1-associated diseases, disorders, or conditions in a subject in need thereof, the methods comprising administering to the subject a therapeutically effective amount of a compound of the disclosure, or a pharmaceutically acceptable salt thereof, or a pharmaceutical composition of the disclosure.

### DETAILED DESCRIPTION

Provided herein are compounds of formula (I) and pharmaceutically acceptable salts thereof that are useful as GLP-1R agonists. The compounds of formula (I) are useful in the treatment or prevention of a disease or condition that would benefit from activation of GLP-1R. For example, the disclosed GLP-1R agonists are useful in therapeutic methods and compositions suitable for use in treating obesity and diabetes.

### Definitions

The articles “a” and “an” are used herein to refer to one or to more than one (i.e., to at least one) of the grammatical object of the article. By way of example, “an element” means one element or more than one element.

The term “heteroatom” is art-recognized and refers to an atom of any element other than carbon or hydrogen. Illustrative heteroatoms include boron, nitrogen, oxygen, phosphorus, sulfur and selenium, and alternatively oxygen, nitrogen or sulfur.

The term “alkyl” as used herein is a term of art and refers to saturated aliphatic groups, including straight-chain alkyl groups, branched-chain alkyl groups, cycloalkyl groups, alkyl substituted cycloalkyl groups, and (cycloalkyl)alkyl groups. In certain embodiments, a straight-chain or branched-chain alkyl has about 30 or fewer carbon atoms in its backbone (e.g., C₁-C₃₀ for straight chain, C₃-C₃₀ for branched chain), and alternatively, about 20 or fewer, or 10 or fewer. In certain embodiments, the term “alkyl” refers to a C₁-C₁₀ alkyl group. In certain embodiments, the term “alkyl” refers to a C₁-C₆ alkyl group, for example a C₁-C₆ straight-chain alkyl group. In certain embodiments, the term “alkyl” refers to a C₃-C₁₂ branched-chain alkyl group. In certain embodiments, the term “alkyl” refers to a C₃-C₈ branched-chain alkyl group. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “cycloalkyl” means mono- or bicyclic saturated carbocyclic rings, each having from 3 to 12 carbon atoms. Certain cycloalkyls have from 5-12 carbon atoms in their ring structure, and may have 6-10 carbons in the ring structure. Preferably, cycloalkyl is (C₃-C₇) cycloalkyl, which represents a monocyclic saturated carbocyclic ring, having from 3 to 7 carbon atoms. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems include bridged bicyclic rings, fused bicyclic rings, and spiro bicyclic rings. Bridged bicyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bridged bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocycloalkyl, a monocyclic heterocycloalkenyl, or a monocyclic heteroaryl. Spiro bicyclic cycloalkyl ring systems contain a monocyclic cycloalkyl ring that shares only one ring atom in common with a second ring. The second ring may be a monocyclic cycloalkyl or a monocyclic heterocycloalkyl. The bridged, fused, or spiro bicyclic cycloalkyl is attached to the parent molecular moiety through any carbon atom contained within the monocyclic cycloalkyl ring. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocycloalkyl, a 5 or 6 membered monocyclic heterocycloalkenyl, or a 5 or 6 membered monocyclic heteroaryl, wherein the fused bicyclic cycloalkyl is optionally substituted.

The term “cycloalkylene” as used herein refers to a divalent cycloalkyl group. In some embodiments, a cycloalkylene may be fused to an arylene or heteroarylene group; i.e., a cycloalkylene may be bonded at two adjacent positions to an arylene or heteroarylene group. In such embodiments, the cycloalkylene is saturated at all atoms except the atoms that are fused to the arylene group.

The term “(cycloalkyl)alkyl” as used herein refers to an alkyl group substituted with one or more cycloalkyl groups. An example of (cycloalkyl)alkyl is cyclohexylmethyl group.

The term “cycloalkenyl” as used herein refers to a cycloalkyl group as defined above that additionally contains at least one carbon-carbon double bond. In certain embodiments, the cycloalkenyl is a mono- or bicyclic carbocyclic ring having at least one carbon-carbon double bond and containing from 3 to 12 carbon atoms. For avoidance of doubt, a cycloalkenyl group is not aromatic. Representative examples of cycloalkenyl include, but are not limited to, cyclohexenyl and cyclopentenyl.

The term “cycloalkynyl” as used herein refers to a cycloalkyl group as defined above that additionally contains at least one carbon-carbon triple bond. In certain embodiments, the cycloalkynyl is a mono- or bicyclic carbocyclic ring having at least one carbon-carbon triple bond and containing from 3 to 12 carbon atoms. For avoidance of doubt, a cycloalkynyl group is not aromatic.

The term “cycloalkenylene” as used herein refers to a divalent cycloalkenyl group. In some embodiments, a cycloalkenylene may be fused to an arylene or heteroarylene group; i.e., a cycloalkenylene may be bonded at two adjacent positions to an arylene or heteroarylene group. In such embodiments, the cycloalkenylene contains at least one saturated carbon atom and at least one carbon-carbon double bond in addition to the atoms that are fused to the arylene group.

The term “heterocycloalkyl” as used herein refers to a radical of a non-aromatic ring system, including, but not limited to, monocyclic, bicyclic, and tricyclic rings, which can be completely saturated or which can contain one or more units of unsaturation, wherein for the avoidance of doubt, the degree of unsaturation does not result in an aromatic ring system, and having 3 to 12 atoms including at least one heteroatom, such as nitrogen, oxygen, or sulfur. Bicyclic heterocycloalkyl ring systems include bridged bicyclic rings, fused bicyclic rings, and spiro bicyclic rings. For purposes of exemplification, which should not be construed as limiting the scope of this invention, the following are examples of heterocyclic rings: aziridinyl, azirinyl, oxiranyl, thiiranyl, thiirenyl, dioxiranyl, diazirinyl, diazepanyl, 1,3-dioxanyl, 1,3-dioxolanyl, 1,3-dithiolanyl, 1,3-dithianyl, imidazolidinyl, isothiazolinyl, isothiazolidinyl, isoxazolinyl, isoxazolidinyl, azetyl, oxetanyl, oxetyl, thietanyl, thietyl, diazetidinyl, dioxetanyl, dithietanyl, dioxalanyl, azepines, azetidinyl, morpholinyl, oxadiazolinyl, oxadiazolidinyl, oxazolinyl, oxazolidinyl, oxopiperidinyl, oxopyrrolidinyl, piperazinyl, piperidinyl, pyranyl, pyrazolinyl, pyrazolidinyl, pyrrolinyl, pyrrolidinyl, quinuclidinyl, thiomorpholinyl, tetrahydropyranyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, trithianyl, tropanyl, 2-azobicyclo[3.1.0]hexane, and 2-oxa-6-azaspiro[3.3]heptane. A heterocycloalkyl group may be optionally substituted by one or more substituents as described below. In certain embodiments, the heterocycloalkyl is attached to the rest of the molecule through a carbon atom in the heterocycloalkyl group, i.e., not through a heteroatom, such as a nitrogen atom, in the heterocycloalkyl group. In certain embodiments, “heterocycloalkyl” is used interchangeably with “heterocyclyl.”

The term “heterocycloalkylene” as used herein refers to a divalent heterocycloalkyl group. In some embodiments, a heterocycloalkylene may be fused to an arylene or heteroarylene group; i.e., a heterocycloalkylene may be bonded at two adjacent positions to an arylene or heteroarylene group. In such embodiments, the heterocycloalkylene is saturated at all atoms except the atoms that are fused to the arylene group. In certain embodiments, “heterocycloalkylene” is used interchangeably with “heterocyclylene.”

The term “heterocycloalkenyl” as used herein refers to a heterocycloalkyl group, as defined above, that additionally contains at least one carbon-carbon double bond. For avoidance of doubt, a heterocycloalkenyl group is not aromatic.

The term “(heterocycloalkyl)alkyl” or (heterocyclyl)alkyl” as used herein refers to an alkyl group substituted with one or more heterocycloalkyl (i.e., heterocyclyl) groups.

The term “heterocycloalkynyl” as used herein refers to a heterocycloalkyl group, as defined above, that additionally contains at least one carbon-carbon triple bond. For avoidance of doubt, a heterocycloalkynyl group is not aromatic.

The term “heterocycloalkenylene” as used herein refers to a divalent heterocycloalkenyl group. In some embodiments, a heterocycloalkenylene may be fused to an arylene or heteroarylene group; i.e., a heterocycloalkenylene may be bonded at two adjacent positions to an arylene or heteroarylene group. In such embodiments, the heterocycloalkenylene contains at least one carbon-carbon double bond in addition to the atoms that are fused to the arylene group.

The term “alkenyl” as used herein means a straight or branched chain hydrocarbon radical containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s).

The term “alkynyl” as used herein means a straight or branched chain hydrocarbon radical containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “alkylene” is art-recognized, and as used herein pertains to a diradical obtained by removing two hydrogen atoms of an alkyl group, as defined above. In certain embodiments an alkylene refers to a disubstituted alkane, i.e., an alkane substituted at two positions with substituents such as those described below. That is, in certain embodiments, a “substituted alkyl” is an “alkylene”.

The term “alkenylene” is art-recognized, and as used herein pertains to a diradical obtained by removing two hydrogen atoms of an alkenyl group, as defined above.

The term “amino” is a term of art and as used herein refers to both unsubstituted and substituted amines, e.g., a moiety that may be represented by the general formulas:

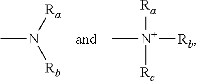

wherein Rₐ, R_{b}, and R_{c} each independently represent a hydrogen, —(CH₂)ₓ—Rₐ, —C(O)-alkyl, —C(O)-alkenyl, where the alkyl or alkenyl may be optionally substituted, or optionally substituted alkyl, alkenyl, alkynyl, aryl, heteroaryl, cycloalkyl, cycloalkenyl, heterocycloalkyl, heterocycloalkenyl, (cycloalkyl)alkyl, (heterocycloalkyl)alkyl, arylalkyl, heteroarylalkyl, alkoxyalkyl, or haloalkyl; or Rₐ and R_{b}, taken together with the N atom to which they are attached form a heterocycle having from 4 to 8 atoms in the ring structure, which may be optionally substituted; R_{d} represents optionally substituted aryl, heteroaryl, cycloalkyl, cycloalkenyl, heterocyclyl or polycyclyl; and x is zero or an integer in the range of 1 to 8. In certain embodiments, only one of Rₐ or R_{b} contains a carbonyl adjacent to the N atom, e.g., Rₐ, R_{b}, and the nitrogen together do not form an imide. In other embodiments, Rₐ and R_{b} (and optionally R_{c}) each independently represent hydrogen, optionally substituted alkyl, optionally substituted alkenyl, or —(CH₂)ₓ—R_{d}. In certain embodiments, the term “amino” refers to —NH₂.

In certain embodiments, the term “alkylamino” refers to —NH(alkyl).

In certain embodiments, the term “dialkylamino” refers to —N(alkyl)₂.

The term “amido”, as used herein, means-NHC(═O)—, wherein the amido group is bound to the parent molecular moiety through the nitrogen or through the carbonyl carbon. Examples of amido include —C(O)NH₂, —C(O)NH(methyl), and alkylamido such as CH₃C(═O)N(H)— and CH₃CH₂C(═O)N(H)—.

The term “acyl” is a term of art and as used herein refers to any group or radical of the form RC(O)— where R is any organic group, e.g., alkyl, aryl, heteroaryl, aralkyl, and heteroaralkyl. Representative acyl groups include acetyl, benzoyl, and malonyl.

The term “aminoalkyl” as used herein refers to an alkyl group substituted with one or more one amino groups. In certain embodiments, the term “aminoalkyl” refers to an aminomethyl group, i.e., —CH₂NH₂.

The term “aminoacyl” is a term of art and as used herein refers to an acyl group substituted with one or more amino groups.

The term “aminothionyl” is a term of art and as used herein refers to any group or radical of the form RC(S)—, wherein R is any organic group, e.g., alkyl, aryl, heteroaryl, arylalkyl, and heteroarylalkyl.

The term “phosphoryl” is a term of art and as used herein may in general be represented by the formula:

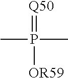

wherein Q50 represents S or O, and R59 represents hydrogen, optionally substituted (C₁-C₆) alkyl or optionally substituted aryl; for example, —P(O)(OMe)- or —P(O)(OH)₂. When used to substitute, e.g., an alkyl, the phosphoryl group of the phosphorylalkyl may be represented by the general formulas:

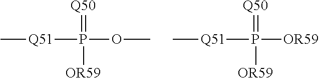

wherein Q50 and R59, each independently, are defined above, and Q51 represents O, S or N; for example, —O—P(O)(OH)OMe or —NH—P(O)(OH)₂. When Q50 is S, the phosphoryl moiety is a “phosphorothioate.”

The term “aminophosphoryl” as used herein refers to a phosphoryl group substituted with at least one amino group, as defined herein; for example, —P(O)(OH)NMe₂.

The term “azide” or “azido”, as used herein, means an —N₃ group.

The term “carbonyl” as used herein refers to —C(═O)—.

The term “thiocarbonyl” as used herein refers to —C(═S)—.

The term “alkylphosphoryl” as used herein refers to a phosphoryl group substituted with at least one alkyl group, as defined herein; for example, —P(O)(OH)Me.

The term “alkylthio” as used herein refers to alkyl-S—. The term “(alkylthio)alkyl” refers to an alkyl group substituted by an alkylthio group.

The term “carboxyl”, as used herein, means a —CO₂H group.

The term “aryl” is a term of art and as used herein refers to includes monocyclic, bicyclic and polycyclic aromatic hydrocarbon groups, for example, benzene, naphthalene, anthracene, and pyrene. Typically, an aryl group contains from 6-10 carbon ring atoms (i.e., (C₆-C₁₀)aryl). The aromatic ring may be optionally substituted at one or more ring positions with one or more substituents as described below. The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, heterocyclyls, heterocycloalkenyls, and/or heterocycloalkynyls. In certain embodiments, the term “aryl” refers to a phenyl group.

The term “arylene” means a diradical obtained by removing two hydrogen atoms of an aryl group, as defined above. Arylene includes, without limitation, 1,2-phenylene, 1,3-phenylene, and 1,4-phenylene, as depicted below:

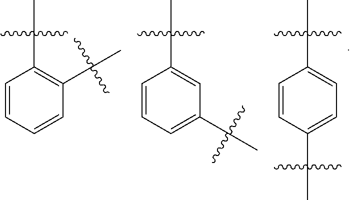 [SMILES: *c1ccccc1* ; *c1cccc(*)c1 ; *c1ccc(*)cc1]

Arylene groups may be optionally substituted at one or more ring positions with one or more substituents, valency permitting, such as the exemplary substituents described below.

The term “heteroaryl” is a term of art and as used herein refers to a monocyclic, bicyclic, or polycyclic aromatic group having 3 to 12 total atoms including one or more heteroatoms such as nitrogen, oxygen, or sulfur in the ring structure. Exemplary heteroaryl groups include azaindolyl, benzo (b) thienyl, benzimidazolyl, benzofuranyl, benzoxazolyl, benzothiazolyl, benzothiadiazolyl, benzotriazolyl, benzoxadiazolyl, furanyl, 1,3-dihydro-2H-imidazol-2-one, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, triazolyl, or triazine, and the like. The “heteroaryl” may be optionally substituted at one or more ring positions with one or more substituents as described below. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, heterocyclyls, heterocycloalkenyls, and/or heterocycloalkynyls.

The term “heteroarylene” as used herein pertains to a diradical obtained by removing two hydrogen atoms of a heteroaryl group, as defined above. Heteroarylene includes, without limitation, the divalent heteroarylene groups depicted below:

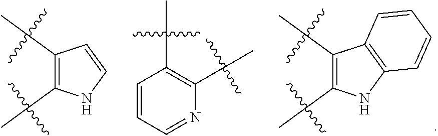

Heteroarylene groups may be optionally substituted at one or more ring positions with one or more substituents, valency permitting, such as the exemplary substituents described below.

The term “aralkyl” or “arylalkyl” is a term of art and as used herein refers to an alkyl group substituted with an aryl group, wherein the moiety is appended to the parent molecule through the alkyl group.

The term “heteroaralkyl” or “heteroarylalkyl” is a term of art and as used herein refers to an alkyl group, as defined herein, substituted with a heteroaryl group, as defined herein, appended to the parent molecular moiety through the alkyl group.

The term “alkoxy” as used herein refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “haloalkoxy” as used herein refers to an alkoxy group, as defined herein, wherein some or all of the hydrogens of the alkyl group are replaced with halogen atoms, as defined herein. Representative examples of haloalkoxy include, but are not limited to, —OCF₃.

The term “alkoxyalkyl” as used herein refers to an alkyl group, as defined herein, substituted by an alkoxy group as defined herein.

The term “alkoxycarbonyl” as used herein means an alkoxy group, as defined herein, appended to the parent molecular moiety through a carbonyl group, represented by —C(═O)—, as defined herein. Representative examples of alkoxycarbonyl include, but are not limited to, methoxycarbonyl, ethoxycarbonyl, and tert-butoxycarbonyl.

The term “alkylcarbonyl”, as used herein, means an alkyl group, as defined herein, appended to the parent molecular moiety through a carbonyl group, represented by —C(═O)—, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “arylcarbonyl”, as used herein, means an aryl group, as defined herein, appended to the parent molecular moiety through a carbonyl group, represented by —C(═O)—, as defined herein. Representative examples of arylcarbonyl include, but are not limited to, benzoyl and (2-pyridinyl)carbonyl.

The term “alkylcarbonyloxy” and “arylcarbonyloxy”, as used herein, means an alkylcarbonyl or arylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. Representative examples of arylcarbonyloxy include, but are not limited to phenylcarbonyloxy.

The term “alkenoxy” or “alkenoxyl” means an alkenyl group, as defined herein, appended to the parent molecular moiety through an oxygen atom. Representative examples of alkenoxyl include, but are not limited to, 2-propen-1-oxyl (i.e., CH₂═CH—CH₂—O—) and vinyloxy (i.e., CH₂═CH—O—).

The term “aryloxy” as used herein means an aryl group, as defined herein, appended to the parent molecular moiety through an oxygen atom.

The term “heteroaryloxy” as used herein means a heteroaryl group, as defined herein, appended to the parent molecular moiety through an oxygen atom.

The term “carbocyclyl” as used herein means a monocyclic or multicyclic (e.g., bicyclic, tricyclic, etc.) hydrocarbon radical containing from 3 to 12 carbon atoms that is completely saturated or has one or more unsaturated bonds, and for the avoidance of doubt, the degree of unsaturation does not result in an aromatic ring system (e.g., phenyl). Examples of carbocyclyl groups include 1-cyclopropyl, 1-cyclobutyl, 2-cyclopentyl, 1-cyclopentenyl, 3-cyclohexyl, 1-cyclohexenyl and 2-cyclopentenylmethyl.

The term “cyano” is a term of art and as used herein refers to —CN.

The term “halo” is a term of art and as used herein refers to —F, —Cl, —Br, or —I.

The term “haloalkyl” as used herein refers to an alkyl group, as defined herein, wherein some or all of the hydrogens are replaced with halogen atoms, as defined herein. Representative examples of haloalkyl include, but are not limited to, trifluoromethyl and fluoroethyl.

The term “hydroxy” is a term of art and as used herein refers to —OH.

The term “hydroxyalkyl”, as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “silyl”, as used herein, includes hydrocarbyl derivatives of the silyl (H₃Si—) group (i.e., (hydrocarbyl)₃Si—), wherein hydrocarbyl groups are univalent groups formed by removing a hydrogen atom from a hydrocarbon, e.g., ethyl, phenyl. The hydrocarbyl groups can be combinations of differing groups which can be varied in order to provide a number of silyl groups, such as trimethylsilyl (TMS), tert-butyldiphenylsilyl (TBDPS), tert-butyldimethylsilyl (TBS/TBDMS), triisopropylsilyl (TIPS), and [2-(trimethylsilyl)ethoxy]methyl (SEM).

The term “silyloxy”, as used herein, means a silyl group, as defined herein, is appended to the parent molecule through an oxygen atom.

Certain compounds contained in compositions of the present invention may exist in particular geometric or stereoisomeric forms. In addition, compounds of the present invention may also be optically active. The present invention contemplates all such compounds, including cis- and trans-isomers, (R)- and(S)-enantiomers, diastereoisomers, atropisomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

In certain embodiments, the optional substituents contemplated in this invention include, for example, halogen, oxo (═O), azide, alkyl, alkenyl, alkynyl, aryl, arylalkyl, heteroaryl, heteroarylalkyl, cycloalkyl, cycloalkenyl, cycloalkynyl, (cycloalkyl)alkyl, heterocycloalkyl, heterocycloalkenyl, heterocycloalkynyl, (heterocycloalkyl)alkyl, hydroxyl, alkoxy, alkenyloxy, alkynyloxy, amino (e.g., —NH₂, —NH(alkyl), or —N(alkyl)₂), aminoalkyl, nitro, sulfhydryl (—SH), imino, amido (e.g., —C(O)NH₂, —C(O)NH(alkyl), —C(O)N(alkyl)₂, —NHC(O)(alkyl), or —N(alkyl)C(O)(alkyl)), phosphoryl, phosphate (e.g., —OP(O)(O-alkyl)₂), phosphonate (e.g., —P(O)(O-alkyl)₂), phosphinate (e.g. —P(O)(alkyl)(O-alkyl)), phosphine (e.g., —P(aryl)₂), carbonyl, carboxyl (—C(O)OH), carboxylalkyl (e.g., -alkylene-(COOH)), silyl, silyloxy, ether (e.g., -alkylene-O(alkyl)), thioether (e.g., -alkylene-S(alkyl)), alkylthio (—S-alkyl), sulfonyl (e.g., —S(O)₂(alkyl)), sulfoxide (e.g., —S(O)(alkyl)), sulfonamido (e.g., —S(O)₂NH₂, —S(O)₂NH(alkyl), or —S(O)₂N(alkyl)₂), sulfinamido (e.g., S(O)NH₂, —S(O)NH(alkyl), or —S(O)N(alkyl)₂), Boc (—C(O)—O—C(CH₃)₃), ketone (e.g., —C(O)(alkyl)), aldehyde (—C(O)H), ester (e.g., -alkylene-C(O)O(alkyl) or —C(O)O(alkyl)), acyloxy (e.g., —OC(O)(alkyl)), haloalkyl, hydroxyalkyl, alkoxyalkyl, haloalkoxy, haloalkoxyalkyl, and cyano (—CN).

As used herein, the term “optionally substituted” or “substituted or unsubstituted” when it precedes a list of chemical moieties means that the list of chemical moieties that follow are each substituted or unsubstituted. For example, “substituted or unsubstituted aryl, heteroaryl, and cycloalkyl” or “optionally substituted aryl, heteroaryl, and cycloalkyl” means substituted or unsubstituted aryl, substituted or unsubstituted heteroaryl, and substituted or unsubstituted cycloalkyl.

The term “vicinal” describes the positional relationship between two substituents, moieties, or functional groups each of which is bonded to one of two adjacent carbon atoms that are bonded to each other (i.e., the two substituents, moieties, or functional groups are in a 1,2-relationship). The methyl groups in 3,4-dimethylheptane are vicinal.

The term “geminal” describes the positional relationship between two substituents, moieties, or functional groups bonded to the same carbon atom (i.e., the two substituents, moieties, or functional groups are in a 1,1-relationship). The methyl groups in 3,3-dimethylheptane are geminal.

The term “hominal” describes the positional relationship between two substituents, moieties, or functional groups each of which is bonded to one of two carbon atoms that themselves are each bonded to a single carbon atom (i.e., the two substituents, moieties, or functional groups are in a 1,3-relationship). The methyl groups in 3,5-dimethylheptane are hominal.

The phrase “protecting group”, as used herein, means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

For purposes of the invention, the chemical elements are identified in accordance with the Periodic Table of the Elements, CAS version, Handbook of Chemistry and Physics, 67th Ed., 1986-87, inside cover.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference). Unless otherwise defined, all technical and scientific terms used herein have the same meaning as commonly understood by one of ordinary skill in the art to which this invention pertains.

The term “GLP-1R” or “GLP-1 receptor” as used herein is meant to include, without limitation, nucleic acids, polynucleotides, oligonucleotides, sense and antisense polynucleotide strands, complementary sequences, peptides, polypeptides, proteins, homologous, and/or orthologous GLP-IR molecules, isoforms, precursors, mutants, variants, derivatives, splice variants, alleles, different species, and active fragments thereof.

The term “GLP-1-associated disease” as used herein is meant to include, without limitation, all those diseases, disorders, or conditions in which modulating glucagon-like peptide-1 (GLP-1) receptor signaling can alter the pathology and/or symptoms and/or progression of the disease, disorder, or condition.

The term “GLP-1 agonist” or “GLP-1R agonist” or “GLP-1RA” as used herein refers to an agonist of the glucagon-like peptide-1 (GLP-1) receptor. GLP-1R agonists enhance glucose-dependent insulin secretion; suppress inappropriately elevated glucagon levels, both in fasting and postprandial states; and slow gastric emptying (Karla et al., Glucagon-like peptide-I receptor agonists in the treatment of type 2 diabetes: Past, present, and future, Indian J. Endocrinol. Metab. 2016 March-April; 20 (2): 254-267). GLP-1R agonists have been shown to treat type 2 diabetes. Examples of GLP-1R agonists include, but are not limited to, albiglutiden (TANZEUM®), dulaglutide (LY2189265, TRULICITY®), efpeglenatide, exenatide (BYETTA®, BYDUREON®, Exendin-4), liraglutide (VICTOZA®, NN2211), lixisenatide (LYXUMIA®), semaglutide (OZEMPIC®), tirzepatide, Orforglipron, Danuglipron, Lotiglipron, Aleniglipron (GSBR-1290), ECC-5004, ZP2929, NNCOI 13-0987, BPI-3016, and TT401. See, also, for example, additional GLP-1 receptor agonists described in U.S. Pat. Nos. 12,187,724; 11,814,381; 10,858,356; 10,370,426; 10,308,700; 10,259,823; 10,208,019; 9,920,106; 9,839,664; 8,129,343; 8,536,122; 7,919,598; 6,414,126; 6,628,343; and RE45313; and international publication numbers WO 2025/006921; WO 2024/169952; WO 2024/125602; WO 2023/169456; WO 2023/016546; WO 2022/052958; WO 2022/048665; WO 2021/155841; WO 2022/017338; WO 2024/153070; WO 2025/002250; WO 2023/220109; WO 2023/220112; WO 2019/239319; WO 2019/239371; WO 2020/103815; WO 2020/207474; WO 2020/234726; WO 2020/044266; WO 2020/117987; WO 2020/263695; WO 2018/056453; and foreign publication numbers CN 116003403; CN 117069743; and CN 117447493.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula I. As another example, the salt may comprise less than one inorganic or organic acid molecule per molecule of base, such as two molecules of compound of Formula I per molecule of tartaric acid.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

The term “treat” as used herein means prevent, halt or slow the progression of, or eliminate a disease or condition in a subject. In certain embodiments “treat” means halt or slow the progression of, or eliminate a disease or condition in a subject. In certain embodiments, “treat” means reduce at least one objective manifestation of a disease or condition in a subject.

The term “effective amount” as used herein refers to an amount that is sufficient to bring about a desired biological effect.

The term “therapeutically effective amount” as used herein refers to an amount that is sufficient to bring about a desired therapeutic effect.

The term “inhibit” as used herein means decrease by an objectively measurable amount or extent. In various embodiments “inhibit” means decrease by at least 5, 10, 20, 30, 40, 50, 60, 70, 80, 90, or 95 percent compared to relevant control. In certain embodiments “inhibit” means decrease 100 percent, i.e., halt or eliminate.

The term “activate” as used herein means increase by an objectively measurable amount or extent. In various embodiments “activate” means increase by at least 5, 10, 20, 30, 40, 50, 60, 70, 80, 90, or 100 percent compared to relevant control.

The term “induce” as used herein includes the promotion of a function, activity, or expression of a particular protein or enzyme. In certain embodiments, a compound of the disclosure induces GLP-1R activity, e.g., increases the activity of GLP-1R. Induction of GLP-1R activity may be measured according to techniques known to those skilled in the art. For example, induction of GLP-1R activity can be measured using a cellular GLP-1R agonist CAMP assay as described below in the Examples.

The term “modulate” or “modulation”, as used herein, refers to a regulation or an adjustment (e.g., increase or decrease) and can include, for example agonism, partial agonism, or antagonism.

The term “subject” as used herein refers to a mammal. In various embodiments, a subject is a mouse, rat, rabbit, cat, dog, pig, sheep, horse, cow, or non-human primate. In certain embodiments, a subject is a human.

As used herein, the symbol [symbol] , when used in connection with a carbon-carbon double bond, indicates that the stereoisomeric configuration of the substituents with respect to the double bond is unknown, or is a mixture of the cis and trans isomers.

### Compounds

In certain aspects, provided herein are compounds of formula (I):

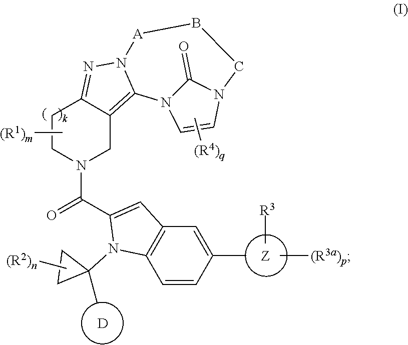

or a pharmaceutically acceptable salts thereof; wherein,
A is optionally substituted arylene, heteroarylene, or heterocycloalkylene;
B is -(alkylene)O(alkylene)-, alkylene, haloalkylene, alkenylene, —O(alkylene)-, (alkylene)O—, —O(alkylene)O—, —O(alkylene)O(alkylene)-, (alkylene)O(alkylene)O—, —S(alkylene)-, -(alkylene)S—, -(alkylene)S(alkylene)-, —S(alkylene)S—, —S(alkylene)S(alkylene)-, -(alkylene)S(alkylene)S—, —NH(alkylene)-, -(alkylene)NH—, -(alkylene)NH(alkylene)-, —NH(alkylene)NH—, —NH(alkylene)NH(alkylene)-, -(alkylene)NH(alkylene)NH—, —N(alkyl)(alkylene)-, (alkylene)N(alkyl)-, or is a bond;
C is optionally substituted arylene, heteroarylene, or heterocycloalkylene;
Ring D is

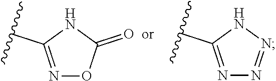

Ring Z is heterocycloalkylene or heteroarylene;
R¹ is independently for each occurrence alkyl, alkoxy, or halo; or two geminal occurrences of R¹ taken together with the carbon atom to which they are attached form an optionally substituted spirocyclic cycloalkyl ring;
R² is independently for each occurrence alkyl, alkoxy, or halo;
R³ is haloalkyl, unsubstituted cycloalkyl, cycloalkyl substituted with alkyl, cycloalkyl substituted with haloalkyl, cycloalkyl substituted with halo, unsubstituted heteroaryl, heteroaryl substituted with alkyl, heteroaryl substituted with halo, unsubstituted heterocycloalkyl, heterocycloalkyl substituted with alkyl, heterocycloalkyl substituted with halo, haloalkoxy, —NH₂, —NH(alkyl), or —N(alkyl)₂;
R^{3a} is independently for each occurrence alkyl, alkoxy, halo, haloalkyl, optionally substituted cycloalkyl, optionally substituted heteroaryl, optionally substituted heterocycloalkyl, haloalkoxy, —NH₂, —NH(alkyl), or —N(alkyl)₂;
R⁴ is independently for each occurrence alkyl, alkoxy, or halo;
k is an integer selected from 0-2;
m is an integer selected from 0-6;
n is an integer selected from 0-4;
p is an integer selected from 0-4; and
q is an integer selected from 0-2.

In certain embodiments, the compound is represented by formula (I-a):

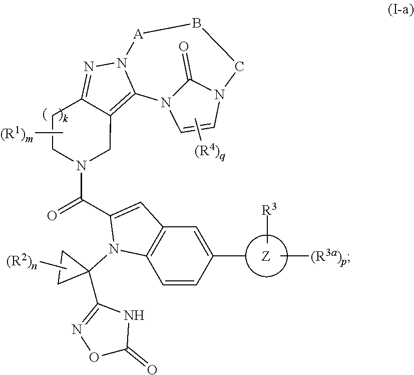

or a pharmaceutically acceptable salt thereof; wherein,
A is optionally substituted arylene, heteroarylene, or heterocycloalkylene;
B is alkylene, haloalkylene, alkenylene, —O(alkylene)-, -(alkylene)O—, (alkylene)O(alkylene)-, —O(alkylene)O—, —O(alkylene)O(alkylene)-, (alkylene)O(alkylene)O—, —S(alkylene)-, -(alkylene)S—, -(alkylene)S(alkylene)-, —S(alkylene)S—, —S(alkylene)S(alkylene)-, -(alkylene)S(alkylene)S—, —NH(alkylene)-, (alkylene)NH—, -(alkylene)NH(alkylene)-, —NH(alkylene)NH—, —NH(alkylene)NH(alkylene)-, -(alkylene)NH(alkylene)NH—, —N(alkyl)(alkylene)-, (alkylene)N(alkyl)-, or is a bond;
C is optionally substituted arylene, heteroarylene, or heterocycloalkylene;
Ring Z is heteroarylene or heterocycloalkylene;
R¹ is independently for each occurrence alkyl, alkoxy, or halo; or
two occurrences of R¹ taken together with the atom(s) to which they are attached form an optionally substituted cycloalkyl ring;
R² is independently for each occurrence alkyl, alkoxy, or halo;
R³ is haloalkyl, unsubstituted cycloalkyl, cycloalkyl substituted with alkyl, cycloalkyl substituted with haloalkyl cycloalkyl substituted with halo, unsubstituted heteroaryl, heteroaryl substituted with alkyl, heteroaryl substituted with halo, unsubstituted heterocycloalkyl, heterocycloalkyl substituted with alkyl, heterocycloalkyl substituted with halo, haloalkoxy, —NH₂, —NH(alkyl), or —N(alkyl)₂;
R^{3a} is independently for each occurrence alkyl, alkoxy, halo, haloalkyl, optionally substituted cycloalkyl, optionally substituted heteroaryl, optionally substituted heterocycloalkyl, haloalkoxy, —NH₂, —NH(alkyl), or —N(alkyl)₂;
R⁴ is independently for each occurrence alkyl, alkoxy, or halo;
k is an integer selected from 0-2;
m is an integer selected from 0-6;
n is an integer selected from 0-4;
p is an integer selected from 0-4; and
q is an integer selected from 0-2.

In certain embodiments, wherein k is 1.

In certain embodiments, the compound is represented by formula (I-b):

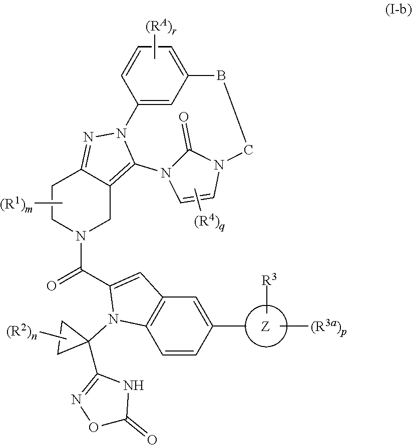

or a pharmaceutically acceptable salt thereof; wherein,
R^{A} is independently for each occurrence alkyl, cycloalkyl, heterocycloalkyl, alkenyl, cycloalkenyl, heterocycloalkenyl, alkynyl, silyl, —NH₂, —NH(alkyl), —N(alkyl)₂, hydroxyl, hydroxyalkyl, alkoxy, —SH, —S-alkyl, —S—S-alkyl, halo, haloalkyl, haloalkoxy, —C(O)H, —C(O)-alkyl, —C(O)NH₂, —C(O)NH(alkyl), —C(O)N(alkyl)₂, —C(O)N(alkyl)(cycloalkyl), —C(O)OH, —OC(O)-alkyl, —C(O)O-alkyl, —C(O)S-alkyl, —S(O)-alkyl, —S(O)₂-alkyl, —S(O)₂-cycloalkyl, —S(O)₂NH₂, —S(O)₂NH(alkyl), —S(O)₂N(alkyl)₂, —S(O)NH₂, —S(O)NH(alkyl), —S(O)N(alkyl)₂, —S(═O)(═NH)(-alkyl), —S(═O)(═N-alkyl)(-alkyl), —CH₂S(O)₂(alkyl), —OP(O)(O-alkyl)₂, —P(O)(alkyl)₂, —CN, —NO₂, —N₃, aryl, or heteroaryl; and
r is an integer selected from 0-4.

In certain embodiments, the compound is represented by formula (I-c):

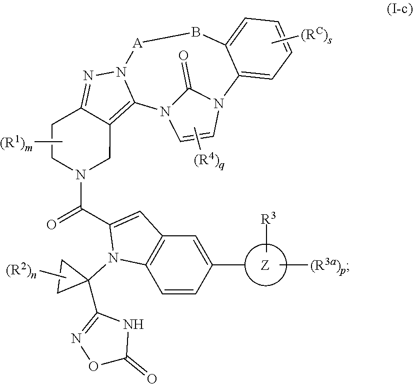

or a pharmaceutically acceptable salt thereof; wherein,
R^{C} is independently for each occurrence alkyl, cycloalkyl, heterocycloalkyl, alkenyl, cycloalkenyl, heterocycloalkenyl, alkynyl, silyl, —NH₂, —NH(alkyl), —N(alkyl)₂, hydroxyl, hydroxyalkyl, alkoxy, —SH, —S-alkyl, —S—S-alkyl, halo, haloalkyl, haloalkoxy, —C(O)H, —C(O)-alkyl, —C(O)NH₂, —C(O)NH(alkyl), —C(O)N(alkyl)₂, —C(O)N(alkyl)(cycloalkyl), —C(O)OH, —OC(O)-alkyl, —C(O)O-alkyl, —C(O)S-alkyl, —S(O)-alkyl, —S(O)₂-alkyl, —S(O)₂-cycloalkyl, —S(O)₂NH₂, —S(O)₂NH(alkyl), —S(O)₂N(alkyl)₂, —S(O)NH₂, —S(O)NH(alkyl), —S(O)N(alkyl)₂, —S(═O)(═NH)(-alkyl), —S(═O)(═N-alkyl)(-alkyl), —CH₂S(O)₂(alkyl), —OP(O)(O-alkyl)₂, —P(O)(alkyl)₂, —CN, —NO₂, —N₃, aryl, or heteroaryl; and
s is an integer selected from 0-4.

In certain embodiments, the compound is represented by formula (I-d):

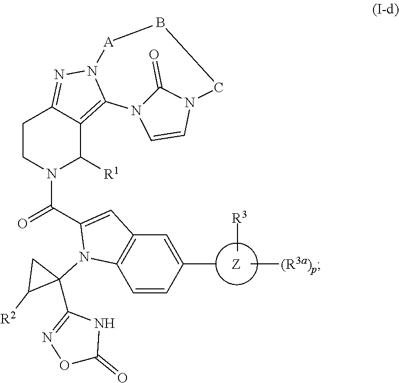

or a pharmaceutically acceptable salt thereof.

In certain embodiments, the compound is represented by formula (I-e):

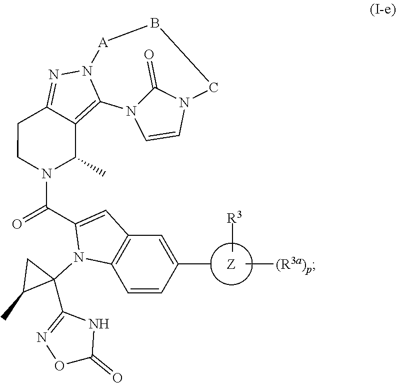

or a pharmaceutically acceptable salt thereof.

In certain embodiments, the compound is represented by formula (I-f):

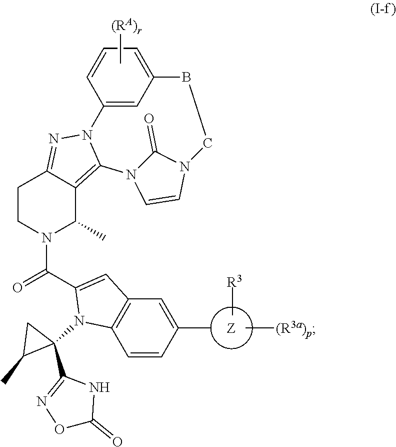

or a pharmaceutically acceptable salt thereof.

In certain embodiments, the compound is represented by formula (I-g):

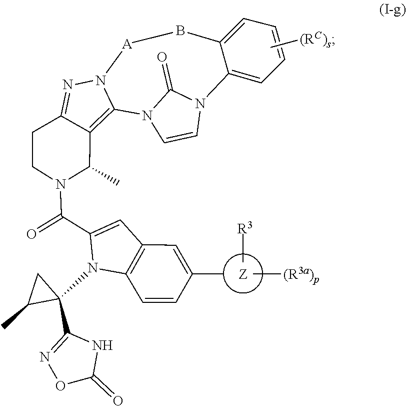

or a pharmaceutically acceptable salt thereof.

In certain embodiments, the compound is represented by formula (I-h):

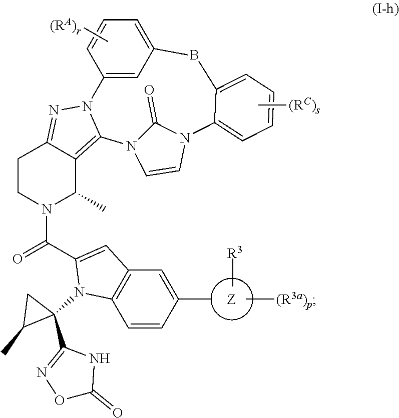

or a pharmaceutically acceptable salt thereof; wherein,
R^{A} is independently for each occurrence alkyl, cycloalkyl, heterocycloalkyl, alkenyl, cycloalkenyl, heterocycloalkenyl, alkynyl, silyl, —NH₂, —NH(alkyl), —N(alkyl)₂, hydroxyl, hydroxyalkyl, alkoxy, —SH, —S-alkyl, —S—S-alkyl, halo, haloalkyl, haloalkoxy, —C(O)H, —C(O)-alkyl, —C(O)NH₂, —C(O)NH(alkyl), —C(O)N(alkyl)₂, —C(O)N(alkyl)(cycloalkyl), —C(O)OH, —OC(O)-alkyl, —C(O)O-alkyl, —C(O)S-alkyl, —S(O)-alkyl, —S(O)₂-alkyl, —S(O)₂-cycloalkyl, —S(O)₂NH₂, —S(O)₂NH(alkyl), —S(O)₂N(alkyl)₂, —S(O)NH₂, —S(O)NH(alkyl), —S(O)N(alkyl)₂, —S(═O)(═NH)(-alkyl), —S(═O)(═N-alkyl)(-alkyl), —CH₂S(O)₂(alkyl), —OP(O)(O-alkyl)₂, —P(O)(alkyl)₂, —CN, —NO₂, —N₃, aryl, or heteroaryl;
R^{C} is independently for each occurrence alkyl, cycloalkyl, heterocycloalkyl, alkenyl, cycloalkenyl, heterocycloalkenyl, alkynyl, silyl, —NH₂, —NH(alkyl), —N(alkyl)₂, hydroxyl, hydroxyalkyl, alkoxy, —SH, —S-alkyl, —S—S-alkyl, halo, haloalkyl, haloalkoxy, —C(O)H, —C(O)-alkyl, —C(O)NH₂, —C(O)NH(alkyl), —C(O)N(alkyl)₂, —C(O)N(alkyl)(cycloalkyl), —C(O)OH, —OC(O)-alkyl, —C(O)O-alkyl, —C(O)S-alkyl, —S(O)-alkyl, —S(O)₂-alkyl, —S(O)₂-cycloalkyl, —S(O)₂NH₂, —S(O)₂NH(alkyl), —S(O)₂N(alkyl)₂, —S(O)NH₂, —S(O)NH(alkyl), —S(O)N(alkyl)₂, —S(═O)(═NH)(-alkyl), —S(═O)(═N-alkyl)(-alkyl), —CH₂S(O)₂(alkyl), —OP(O)(O-alkyl)₂, —P(O)(alkyl)₂, —CN, —NO₂, —N₃, aryl, or heteroaryl;
r is an integer selected from 0-4; and
s is an integer selected from 0-4.

In certain embodiments, A is optionally substituted phenylene, pyridinylene, pyridazinylene, pyrimidinylene, or pyrazinylene. In more particular embodiments, A is optionally substituted phenylene.

In certain embodiments, A is optionally substituted by one or more R^{A}, wherein R^{A} is independently for each occurrence alkyl, cycloalkyl, heterocycloalkyl, alkenyl, cycloalkenyl, heterocycloalkenyl, alkynyl, silyl, —NH₂, —NH(alkyl), —N(alkyl)₂, hydroxyl, hydroxyalkyl, alkoxy, —SH, —S-alkyl, —S—S-alkyl, halo, haloalkyl, haloalkoxy, —C(O)H, —C(O)-alkyl, —C(O)NH₂, —C(O)NH(alkyl), —C(O)N(alkyl)₂, —C(O)N(alkyl)(cycloalkyl), —C(O)OH, —OC(O)-alkyl, —C(O)O-alkyl, —C(O)S-alkyl, —S(O)-alkyl, —S(O)₂-alkyl, —S(O)₂-cycloalkyl, —S(O)₂NH₂, —S(O)₂NH(alkyl), —S(O)₂N(alkyl)₂, —S(O)NH₂, —S(O)NH(alkyl), —S(O)N(alkyl)₂, —S(═O)(═NH)(-alkyl), —S(═O)(═N-alkyl)(-alkyl), —CH₂S(O)₂(alkyl), —OP(O)(O-alkyl)₂, —P(O)(alkyl)₂, —CN, —NO₂, —N₃, aryl, or heteroaryl.

In certain embodiments, each R^{A} is independently alkyl, haloalkyl, cycloalkyl, alkoxy, or halo.

In certain embodiments, R^{A} is independently methyl, trifluoromethyl, cyclopropyl, methoxy, fluoro, or chloro. In more particular embodiments, each R^{A} is independently methyl, fluoro, or chloro.

In certain embodiments, r is an integer selected from 1 or 2. In alternative embodiments, r is 0.

In certain embodiments, -A-* is

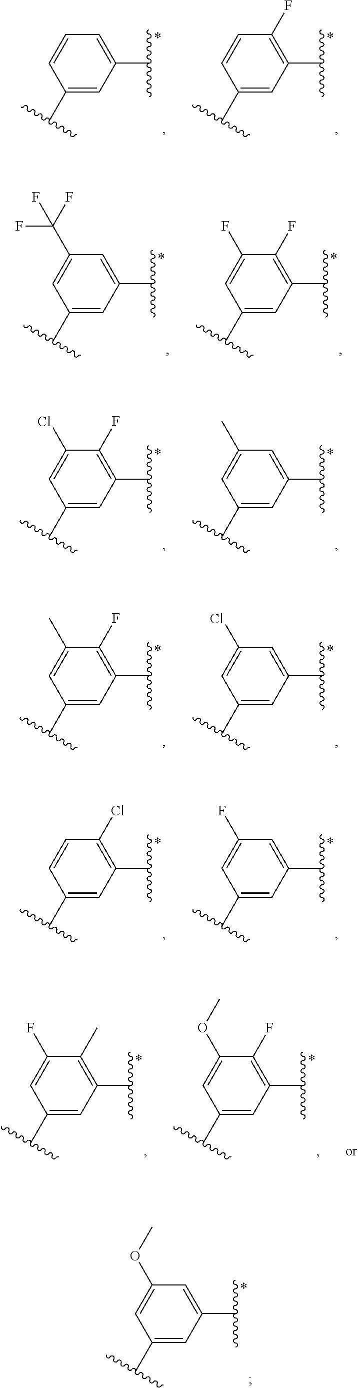

wherein the asterisk (*) indicates the point of attachment to B.

In more particular embodiments, -A-* is

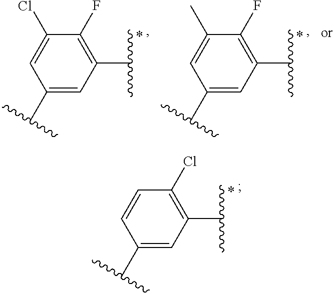

wherein the asterisk (*) indicates the point of attachment to B.

In certain embodiments, B is *—CH₂CH₂—, *—CH₂CH₂CH₂—, *—CH₂CH₂CH₂CH₂—, *—CH₂CH₂CH₂CH₂CH₂—, *—CF₂CH₂CH₂CH₂—, *—CH(CH₃)OCH₂—, *—OCH₂—, *—CH₂O—, *—OCH₂CH₂—, *—CH₂OCH₂—, *—CH₂CH₂O—, *—OCH₂CH₂CH₂—, *—CH₂OCH₂CH₂—, *—CH₂CH₂OCH₂—, *—CH₂CH₂CH₂O—, *—OCH₂CH₂O—, *—OCH₂CH₂CH₂CH₂—, *—CH₂OCH₂CH₂CH₂—, *—CH₂CH₂OCH₂CH₂—, *—CH₂CH₂CH₂OCH₂—, * CH₂CH₂CH₂CH₂O—, *—SCH₂—, *—CH₂S—, *—SCH₂CH₂—, *—CH₂SCH₂—, *—CH₂CH₂S—, *—SCH₂CH₂CH₂—, *—CH₂SCH₂CH₂—, *—CH₂CH₂SCH₂—, *—CH₂CH₂CH₂S—, *—SCH₂CH₂S—, *—SCH₂CH₂CH₂CH₂—, *—CH₂SCH₂CH₂CH₂—, *—CH₂CH₂SCH₂CH₂—, *—CH₂CH₂CH₂SCH₂—, CH₂CH₂CH₂CH₂S—, *—CH═CH—, *—CH—CHCH₂—, *—CH₂CH—CH—, *—CH—CHCH₂CH₂—, *_CH₂CH—CHCH₂—, *—CH₂CH₂CH—CH—, *—CH═CHCH₂CH₂CH₂—, *—CH₂CH═CHCH₂CH₂—, *—CH₂CH₂CH═CHCH₂—, *—CH₂CH₂CH₂CH═CH—, *—N(CH₃)CH₂CH₂—, or *—CH₂CH₂N(CH₃)—;

wherein the asterisk (*) indicates the point of attachment to A.

In more particular embodiments, B is *—CH₂CH₂—, *—CH₂CH₂CH₂—, *—CH₂CH₂CH₂CH₂—, *—CF₂CH₂CH₂CH₂—, *—CH═CHCH₂—, *—CH₂CH═CHCH₂—, *—CH₂O—, *—OCH₂—, *—CH₂OCH₂—, *—OCH₂CH₂—, *—OCH₂CH₂CH₂—, *—CH₂OCH₂CH₂—, *—CH₂CH₂OCH₂—, *—CH₂CH₂CH₂O—, *—OCH₂CH₂O—, *—CH(CH₃)OCH₂—, *—N(CH₃)CH₂CH₂—, *—SCH₂CH₂—, *—SCH₂CH₂CH₂—;

wherein the asterisk (*) indicates the point of attachment to A.

In further embodiments, B is

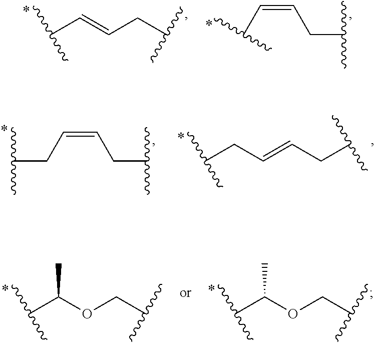

wherein the asterisk (*) indicates the point of attachment to A.

In some embodiments, B is *—CH₂OCH₂—;

wherein the asterisk (*) indicates the point of attachment to A.

In certain embodiments, C is optionally substituted phenylene, pyridinylene, pyridazinylene, pyrimidinylene, pyrazinylene, indolylene, indazolylene, benzimidazolylene, or 2-hydroxybenzimidazolylene. In more particular embodiments, C is optionally substituted phenylene.

In certain embodiments, C is optionally substituted by one or more R^{C}, wherein R^{C} is independently for each occurrence alkyl, cycloalkyl, heterocycloalkyl, alkenyl, cycloalkenyl, heterocycloalkenyl, alkynyl, silyl, —NH₂, —NH(alkyl), —N(alkyl)₂, hydroxyl, hydroxyalkyl, alkoxy, —SH, —S-alkyl, —S—S-alkyl, halo, haloalkyl, haloalkoxy, —C(O)H, —C(O)-alkyl, —C(O)NH₂, —C(O)NH(alkyl), —C(O)N(alkyl)₂, —C(O)N(alkyl)(cycloalkyl), —C(O)OH, —OC(O)-alkyl, —C(O)O-alkyl, —C(O)S-alkyl, —S(O)-alkyl, —S(O)₂-alkyl, —S(O)₂-cycloalkyl, —S(O)₂NH₂, —S(O)₂NH(alkyl), —S(O)₂N(alkyl)₂, —S(O)NH₂, —S(O)NH(alkyl), —S(O)N(alkyl)₂, —S(═O)(═NH)(-alkyl), —S(═O)(═N-alkyl)(-alkyl), —CH₂S(O)₂(alkyl), —OP(O)(O-alkyl)₂, —P(O)(alkyl)₂, —CN, —NO₂, —N₃, aryl, or heteroaryl.

In more particular embodiments, each R^{C} is independently alkyl, alkoxy, halo, —C(O)NH(alkyl), —C(O)N(alkyl)(cycloalkyl), —S(O)₂-alkyl, —S(O)₂-cycloalkyl, —CN, or heterocycloalkyl.

In further embodiments, each R^{C} is independently methyl, methoxy, chloro, fluoro, —C(O)NHMe, —S(O)₂Me, —S(O)₂Et, —CN,

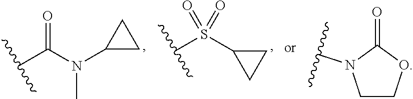

In some embodiments, R^{C} is fluoro.

In certain embodiments, s is an integer selected from 1 or 2.

In alternative embodiments, s is 0.

In certain embodiments, *—C— is

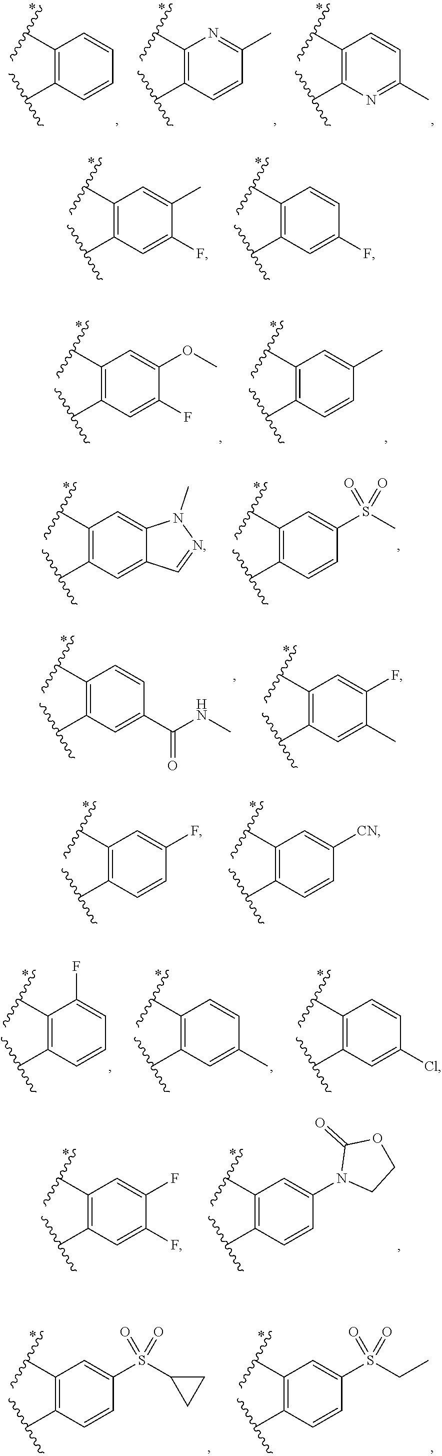

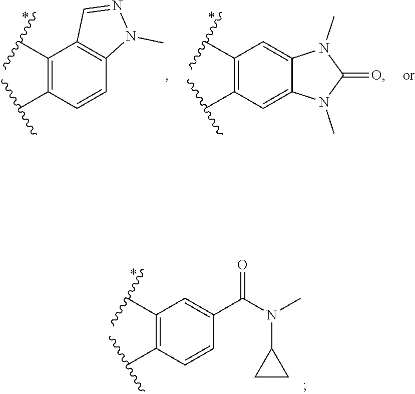

wherein the asterisk (*) indicates the point of attachment to B.

In more particular embodiments, *—C— is

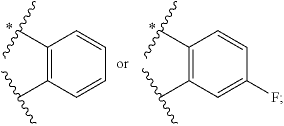 [SMILES: *c1ccccc1* ; *c1ccc(F)cc1*]

wherein the asterisk (*) indicates the point of attachment to B.

In certain embodiments, ring Z is heterocycloalkylene.

In more particular embodiments, ring Z is tetrahydropyranylene, morpholinylene, 8-oxa-3-azabicyclo[3.2.1]octanylene, 2,3-dihydro-1H-pyrido[2,3-b][1,4]oxazinylene, 6,7-dihydro-5H-cyclopenta[b]pyridinylene, 2-oxa-6-azaspiro[3.3]heptanylene, or 1,4-oxazepanylene. In some embodiments, ring Z is morpholinylene.

In certain embodiments, ring Z is heteroarylene.

In more particular embodiments, ring Z is pyridinylene, pyrimidinylene, pyridazinylene, pyrazinylene, imidazolylene, 2,3-dihydro-1H-pyrido[2,3-b][1,4]oxazinylene, 6,7-dihydro-5H-cyclopenta[b]pyridinylene, 1H-pyrrolo[2,3-b]pyridinylene, 1,2,3-triazolylene, or pyrazolylene. In some embodiments, ring Z is pyridinylene.

In certain embodiments, R³ is —NH₂, —N(Me)(Et), NMe₂, —CHF₂, —CF₃, —CH₂CF₃, —CF₂CH₃, —CF₂CF₃, —OCHF₂, —OCF₃,

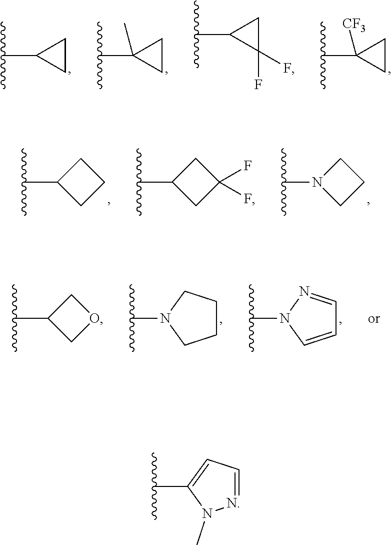

In more particular embodiments, R³ is CF₃, —CF₂CH₃, or

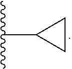 [SMILES: *C1CC1]

In certain embodiments, R^{3a} is alkyl or halo. In more particular embodiments, R^{3a} is methyl or fluoro.

In certain embodiments, p is 1. In alternative embodiments, p is 0.

In certain embodiments,

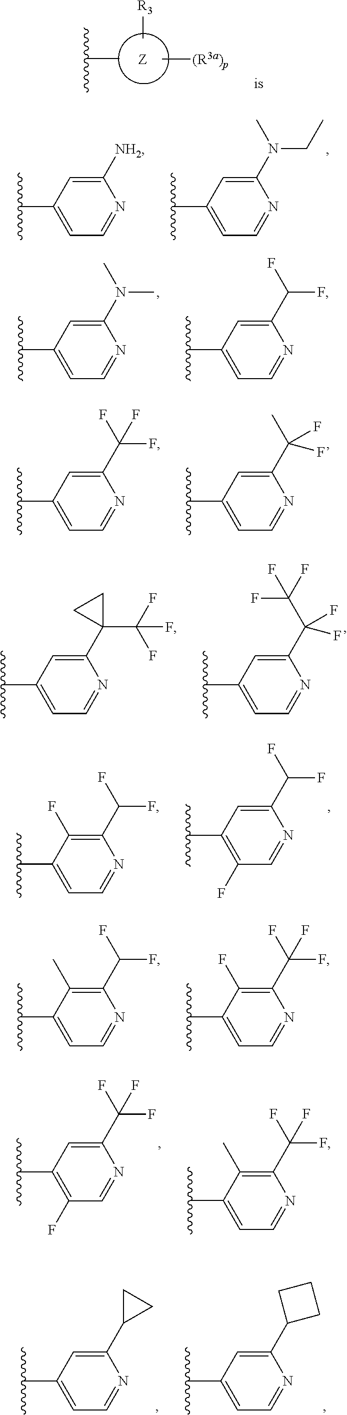

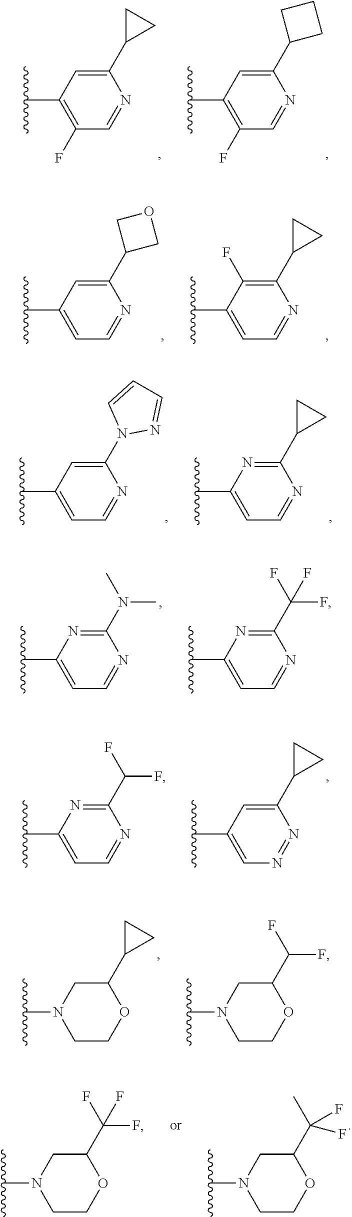

In more particular embodiments,

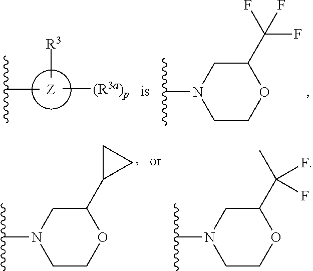

In further embodiments,

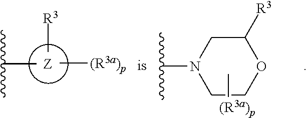

In some embodiments,

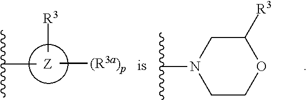

In certain embodiments, R¹ is alkyl. In more particular embodiments, R¹ is methyl.

In certain embodiments, two geminal occurrences of R¹ taken together with the carbon atom to which they are attached form an optionally substituted spirocyclic cycloalkyl ring. In more particular embodiments, two geminal occurrences of R¹ taken together with the carbon atom to which they are attached form a spirocyclic cyclopropyl ring.

In certain embodiments, m is an integer selected from 1-3. In more particular embodiments, m is 1.

In certain embodiments, m is an integer selected from 3 and 4, wherein two geminal occurrences of R¹ taken together with the carbon atom to which they are attached form an optionally substituted spirocyclic cycloalkyl ring, and the remaining occurrences of R¹ are each independently alkyl, alkoxy, or halo. In more particular embodiments, m is 3, two geminal occurrences of R¹ taken together with the carbon atom to which they are attached form a spirocyclic cyclopropyl ring, and the remaining occurrence of R¹ is methyl.

In certain embodiments, R² is alkyl. In more particular embodiments, R² is methyl.

In certain embodiments, n is 1.

In certain embodiments, q is 0.

In certain embodiments, ring D is

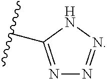 [SMILES: *c1nnn[nH]1]

In certain embodiments, the compound is selected from:

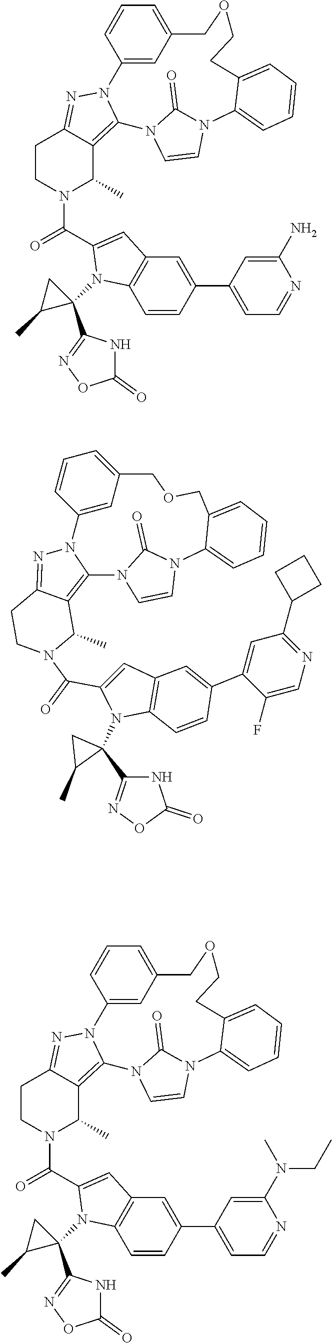 [SMILES: C[C@H]1c2c(nn3c2-n2ccn(c2=O)-c2ccccc2CCOCc2cccc-3c2)CCN1C(=O)c1cc2cc(-c3ccnc(N)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3cc(C4CCC4)ncc3F)ccc2n1[C@@]1(c2noc(=O)[nH]2)C[C@@H]1C ; CCN(C)c1cc(-c2ccc3c(c2)cc(C(=O)N2CCc4nn5c(c4[C@@H]2C)-n2ccn(c2=O)-c2ccccc2CCOCc2cccc-5c2)n3[C@@]2(c3noc(=O)[nH]3)C[C@@H]2C)ccn1]

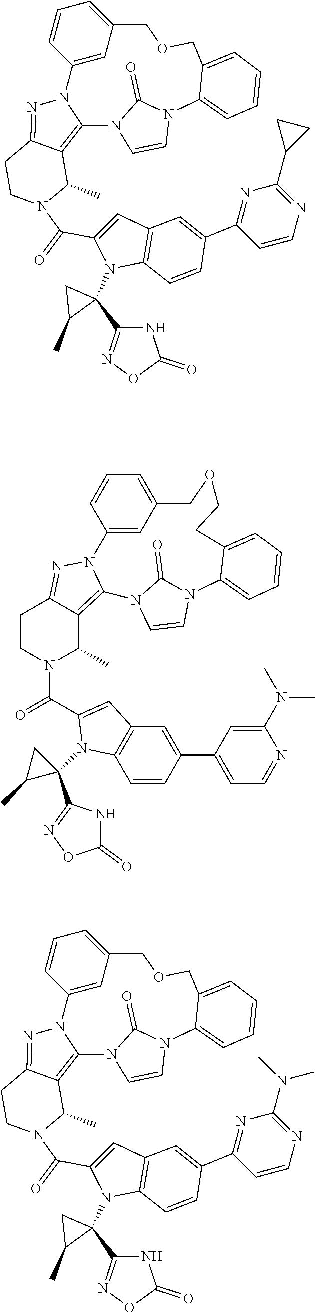

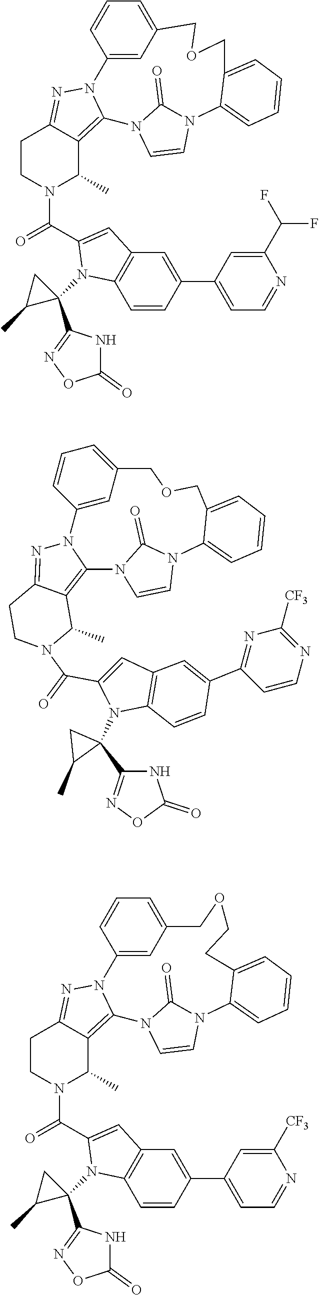 [SMILES: C[C@H]1c2c(nn3c2-n2ccn(c2=O)-c2ccccc2COCc2cccc-3c2)CCN1C(=O)c1cc2cc(-c3ccnc(C(F)F)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3ccnc(C(F)(F)F)n3)ccc2n1[C@@]1(c2noc(=O)[nH]2)C[C@@H]1C ; C[C@H]1c2c(nn3c2-n2ccn(c2=O)-c2ccccc2CCOCc2cccc-3c2)CCN1C(=O)c1cc2cc(-c3ccnc(C(F)(F)F)c3)ccc2n1[C@@]1(c2noc(=O)[nH]2)C[C@@H]1C]

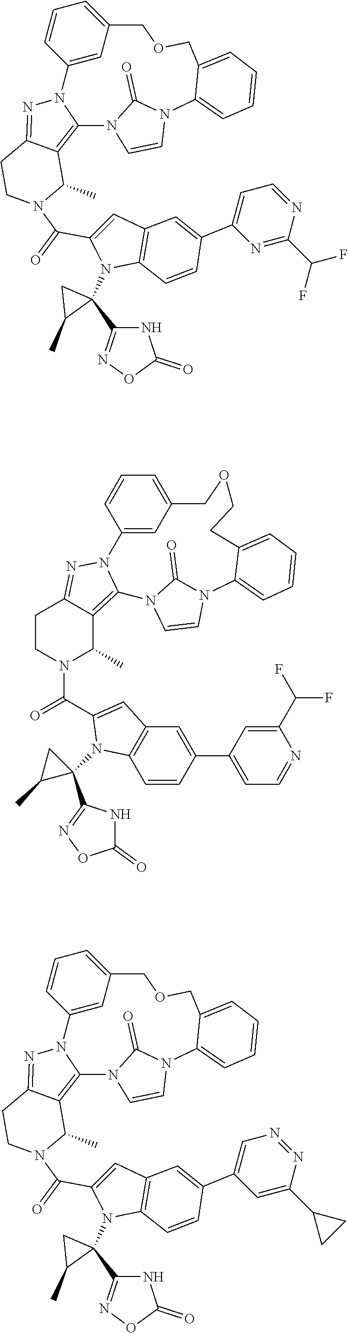 [SMILES: C[C@H]1c2c(nn3c2-n2ccn(c2=O)-c2ccccc2COCc2cccc-3c2)CCN1C(=O)c1cc2cc(-c3ccnc(C(F)F)n3)ccc2n1[C@@]1(c2noc(=O)[nH]2)C[C@@H]1C ; C[C@H]1c2c(nn3c2-n2ccn(c2=O)-c2ccccc2CCOCc2cccc-3c2)CCN1C(=O)c1cc2cc(-c3ccnc(C(F)F)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3cnnc(C4CC4)c3)ccc2n1[C@@]1(c2noc(=O)[nH]2)C[C@@H]1C]

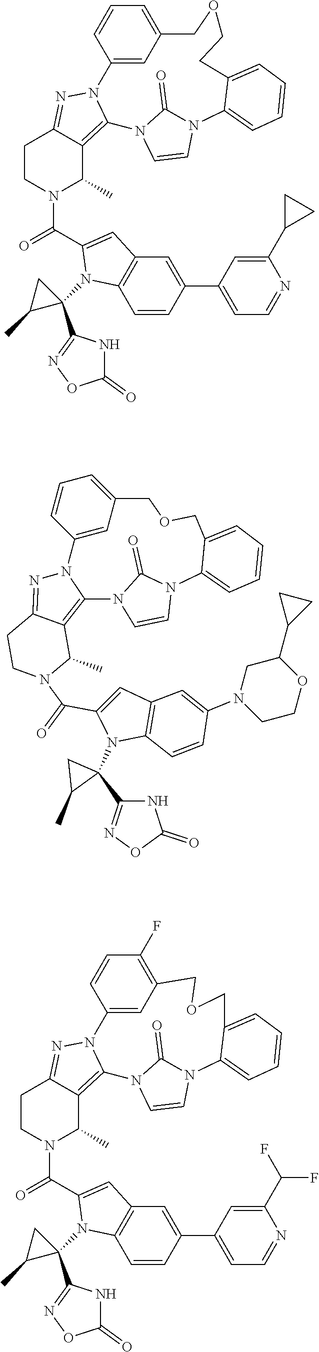 [SMILES: C[C@H]1c2c(nn3c2-n2ccn(c2=O)-c2ccccc2CCOCc2cccc-3c2)CCN1C(=O)c1cc2cc(-c3ccnc(C4CC4)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(N3CCOC(C4CC4)C3)ccc2n1[C@@]1(c2noc(=O)[nH]2)C[C@@H]1C ; C[C@H]1c2c(nn3c2-n2ccn(c2=O)-c2ccccc2COCc2cc-3ccc2F)CCN1C(=O)c1cc2cc(-c3ccnc(C(F)F)c3)ccc2n1[C@@]1(c2noc(=O)[nH]2)C[C@@H]1C]

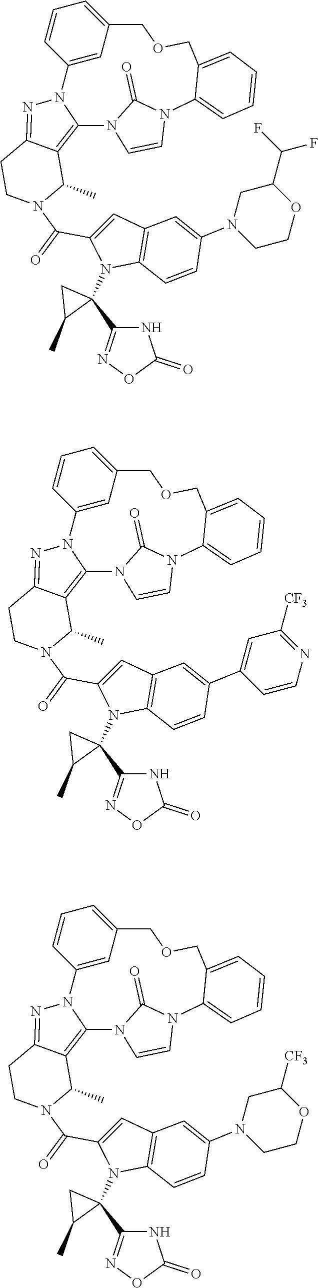 [SMILES: C[C@H]1c2c(nn3c2-n2ccn(c2=O)-c2ccccc2COCc2cccc-3c2)CCN1C(=O)c1cc2cc(N3CCOC(C(F)F)C3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3ccnc(C(F)(F)F)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(N3CCOC(C(F)(F)F)C3)ccc2n1[C@@]1(c2noc(=O)[nH]2)C[C@@H]1C]

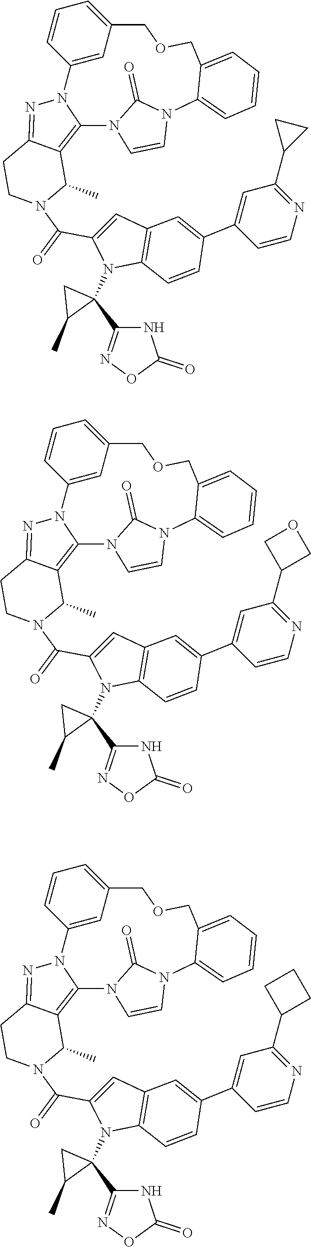 [SMILES: C[C@H]1c2c(nn3c2-n2ccn(c2=O)-c2ccccc2COCc2cccc-3c2)CCN1C(=O)c1cc2cc(-c3ccnc(C4CC4)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3ccnc(C4COC4)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3ccnc(C4CCC4)c3)ccc2n1[C@@]1(c2noc(=O)[nH]2)C[C@@H]1C]

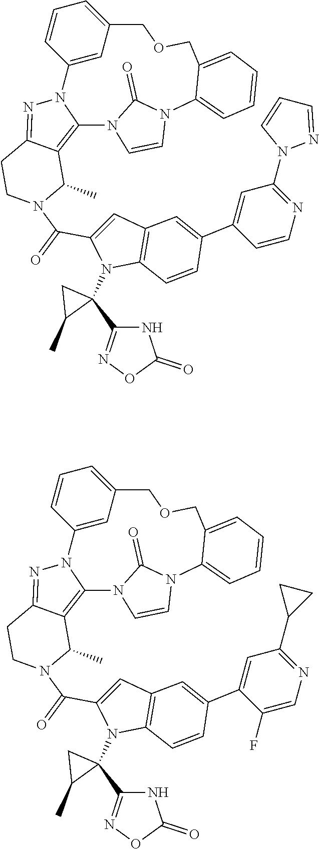 [SMILES: C[C@H]1c2c(nn3c2-n2ccn(c2=O)-c2ccccc2COCc2cccc-3c2)CCN1C(=O)c1cc2cc(-c3ccnc(-n4cccn4)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3cc(C4CC4)ncc3F)ccc2n1[C@@]1(c2noc(=O)[nH]2)C[C@@H]1C]

or a pharmaceutically acceptable salt thereof.

In certain embodiments, the compound is selected from:

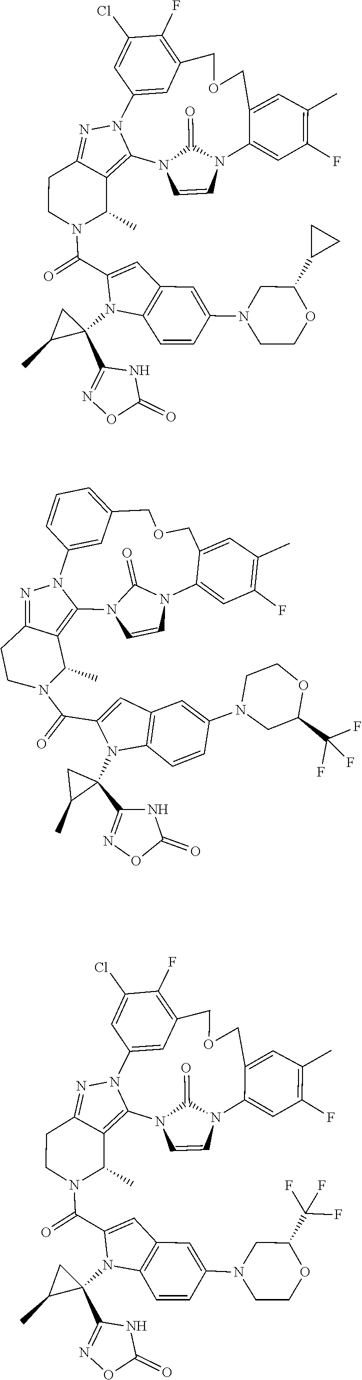

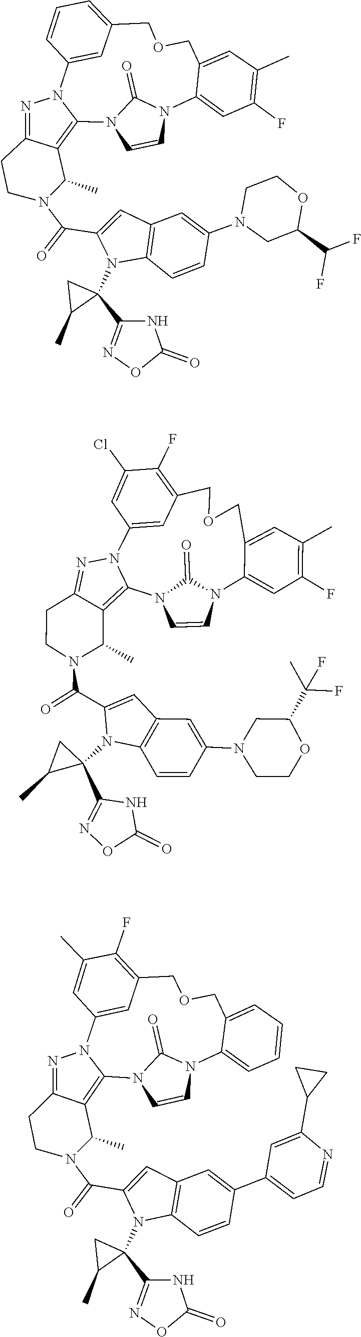

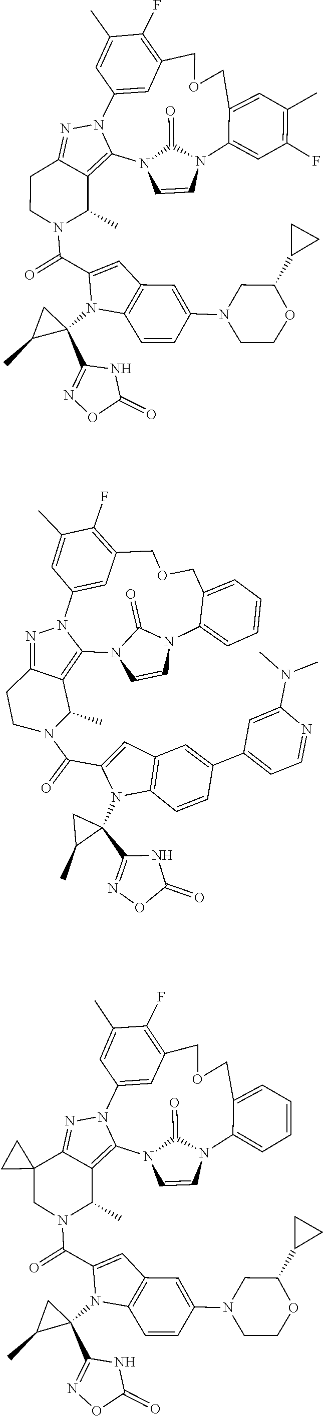

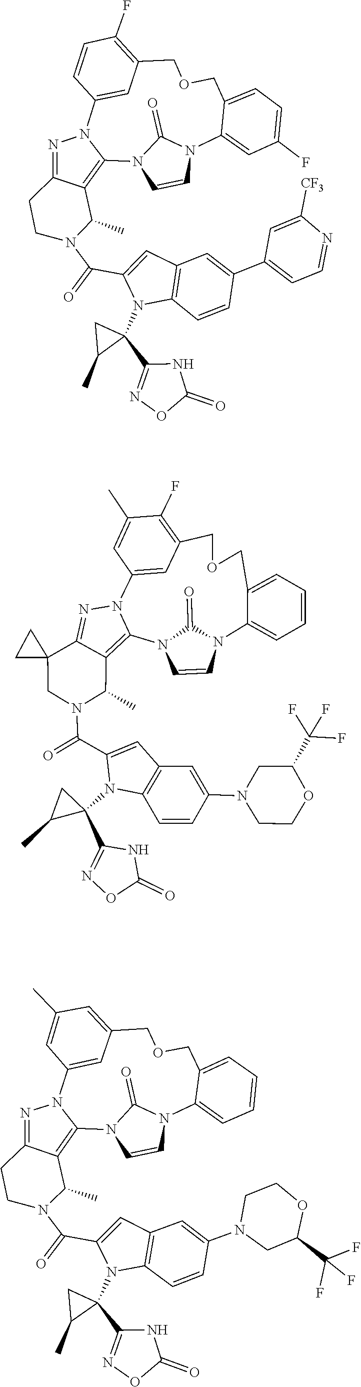

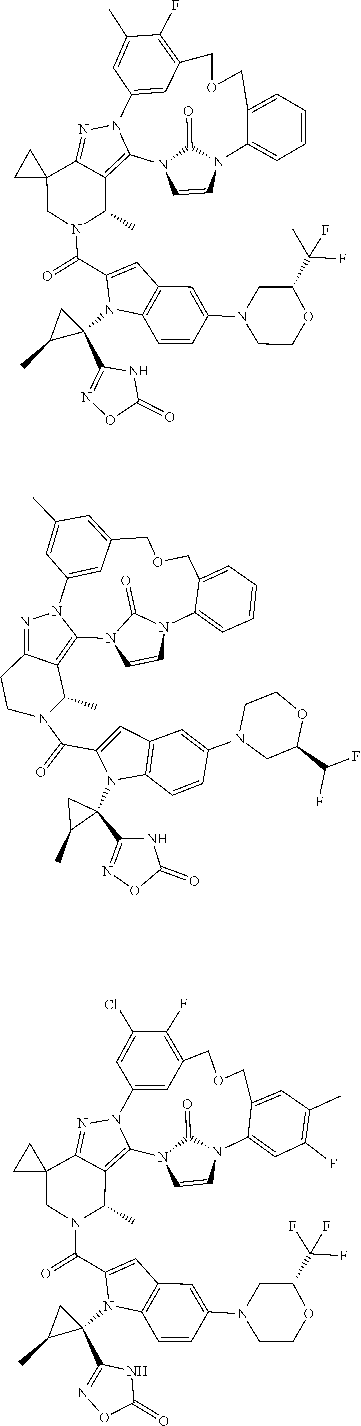

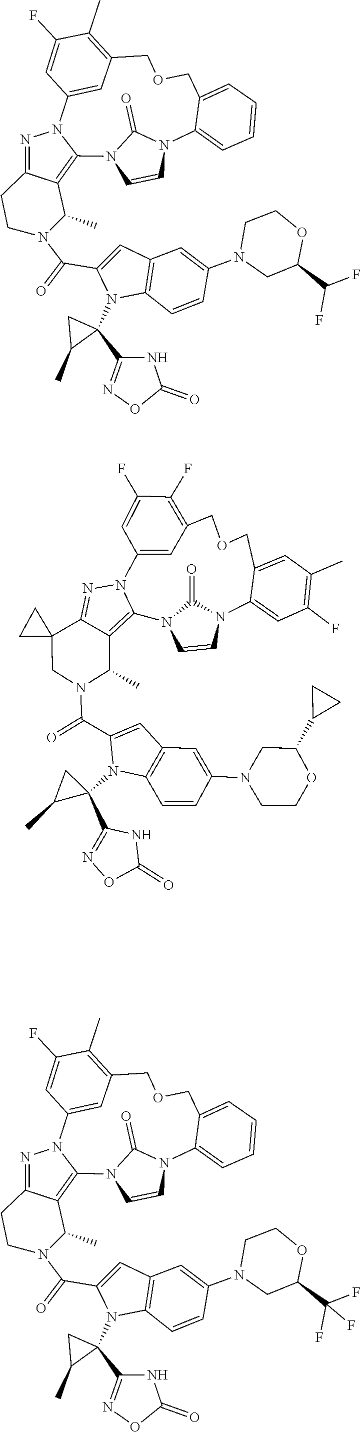

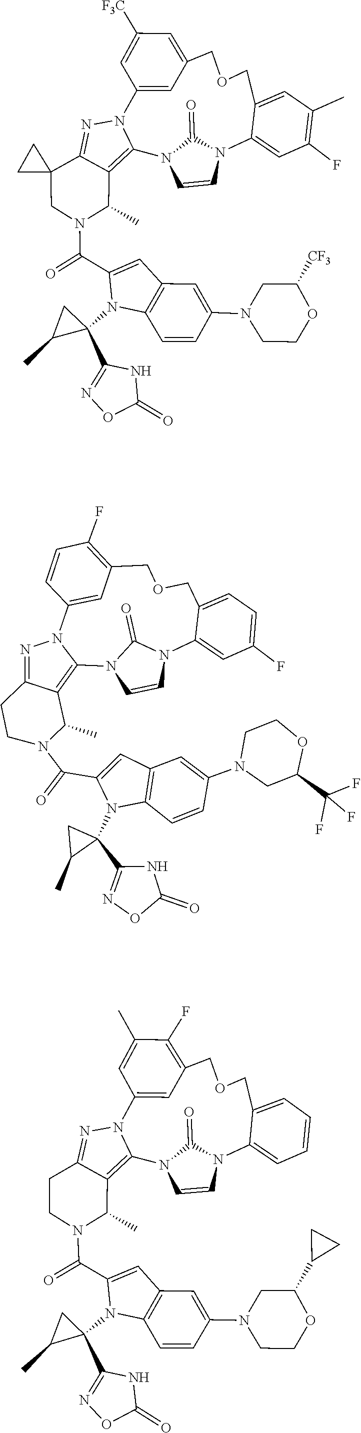

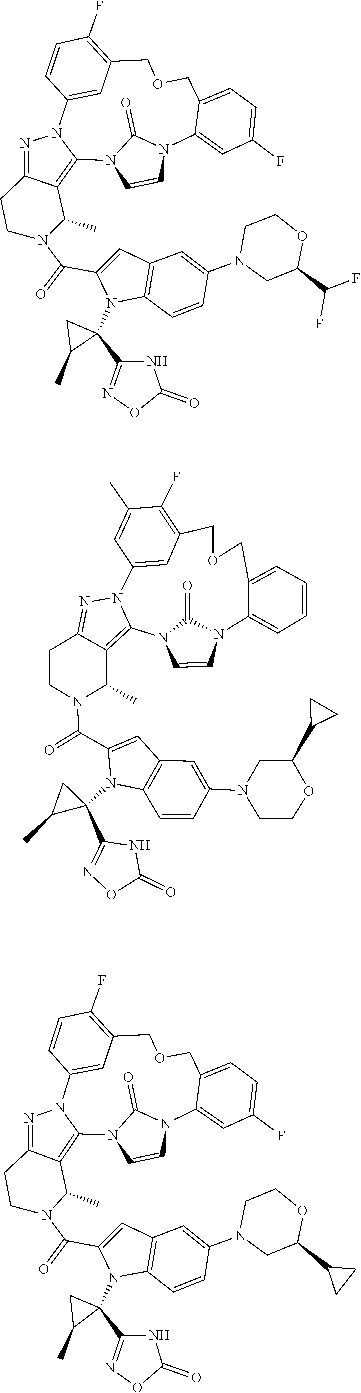

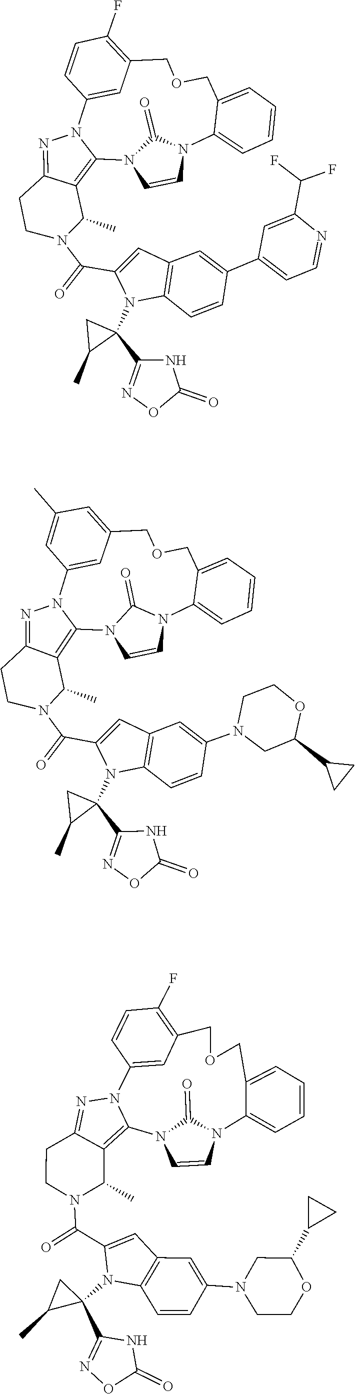

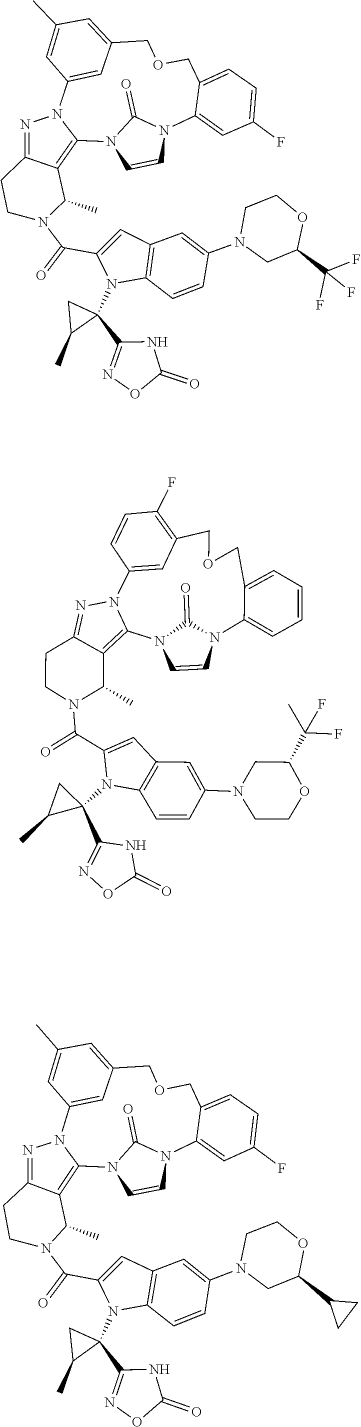

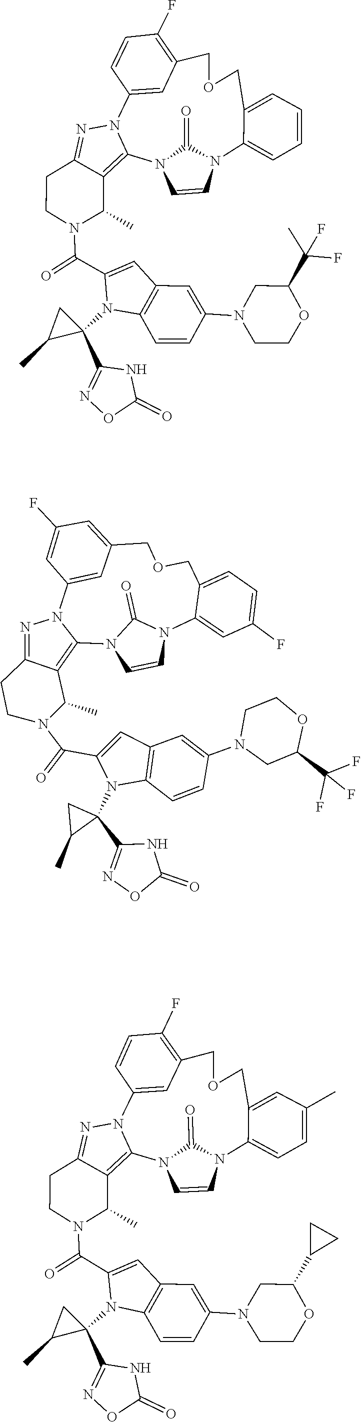

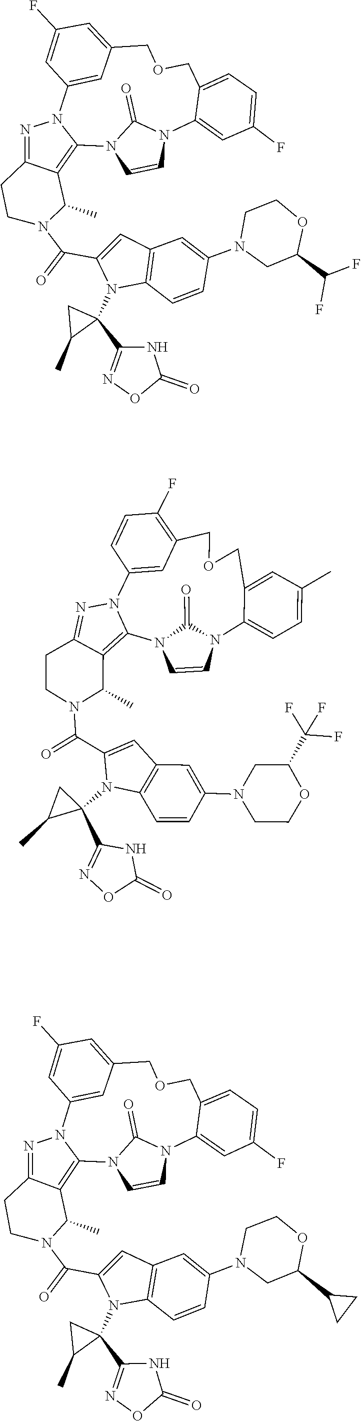

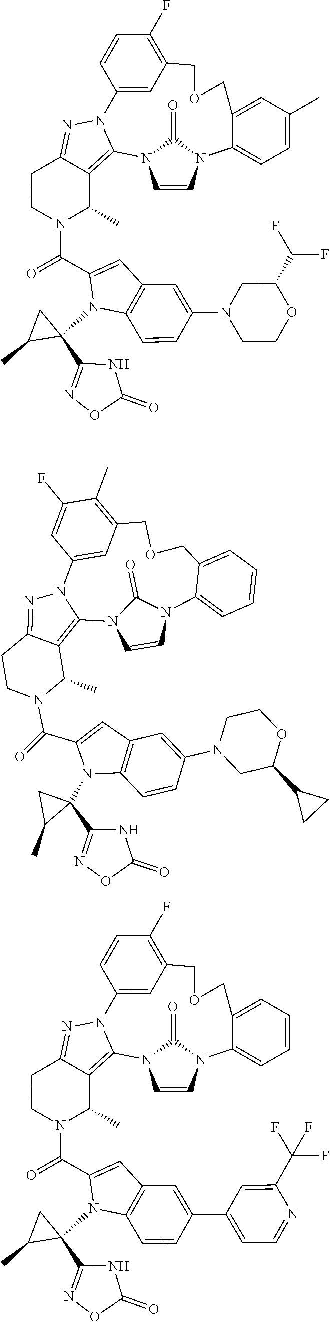

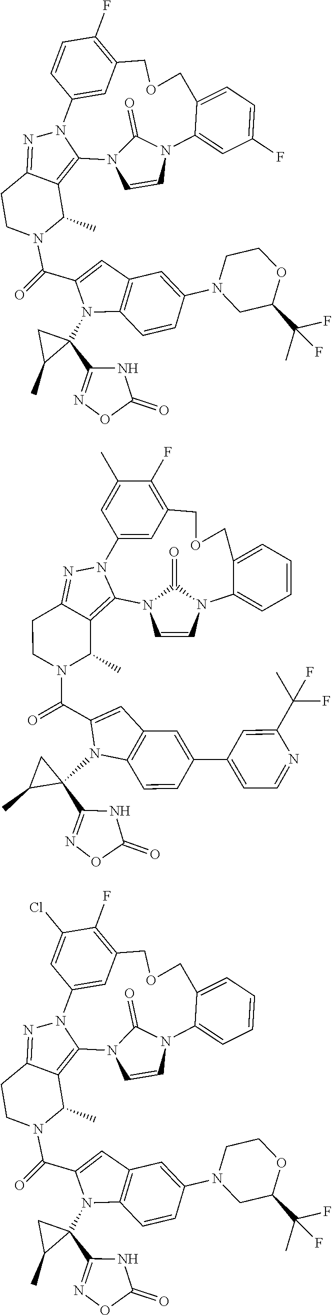

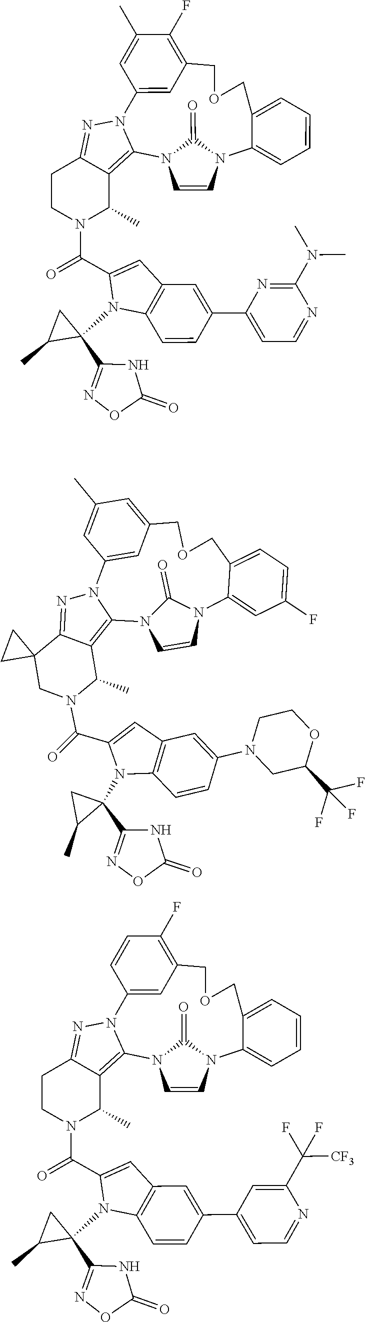

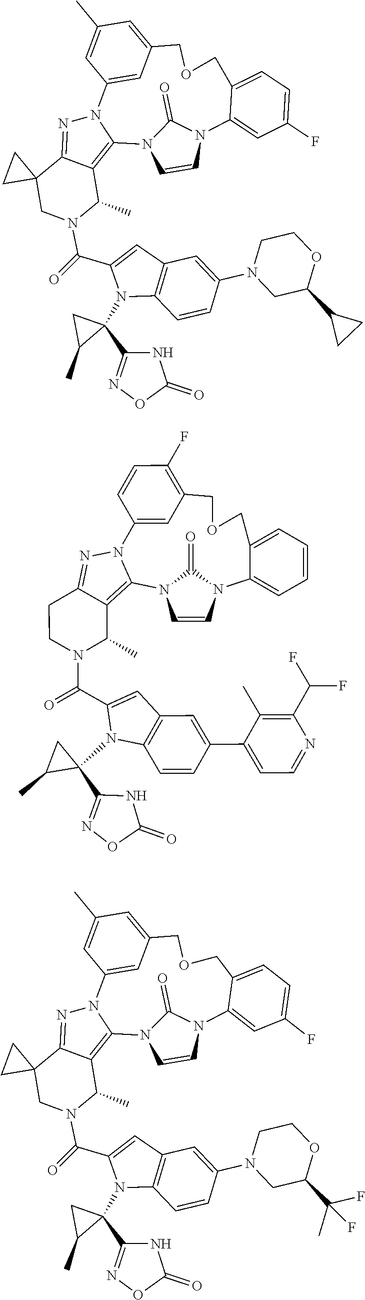

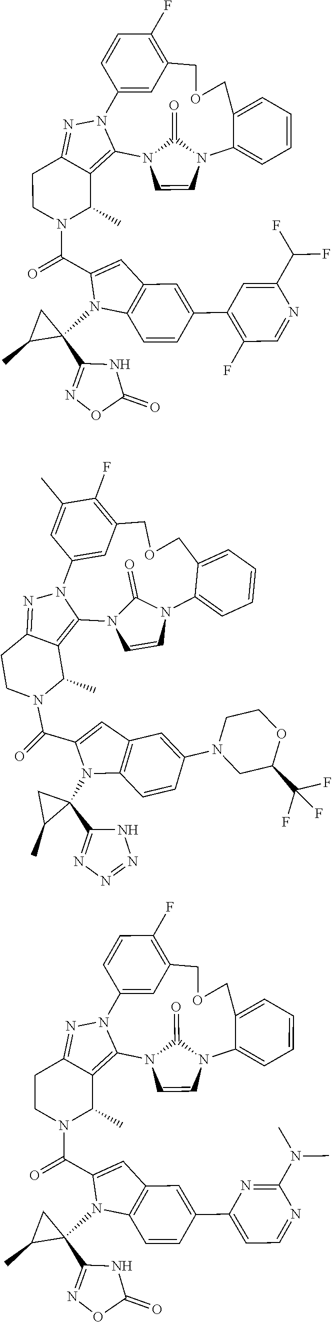

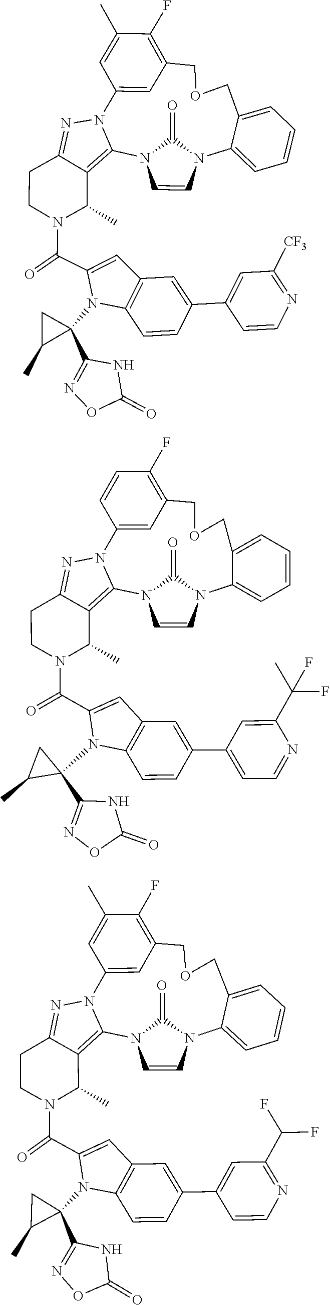

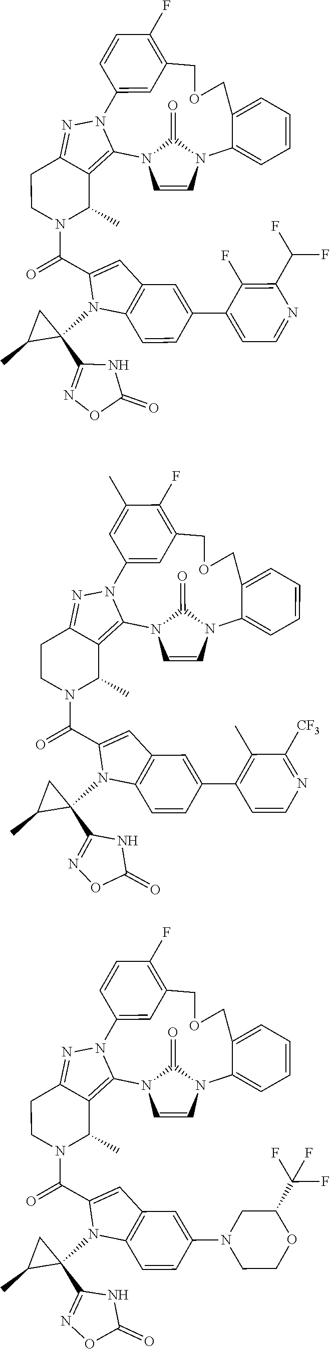

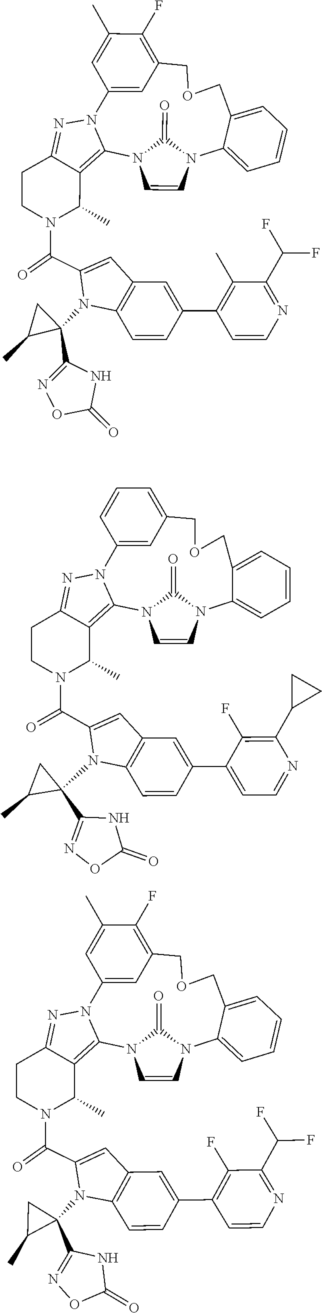

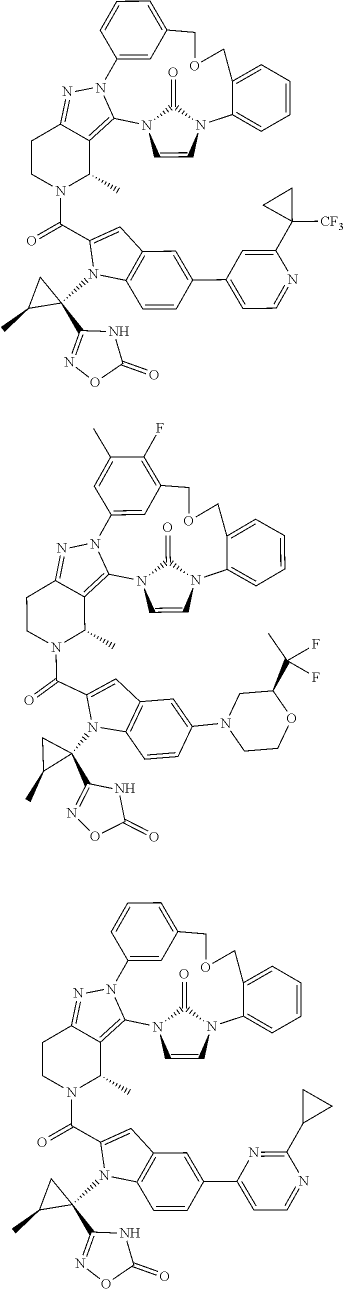

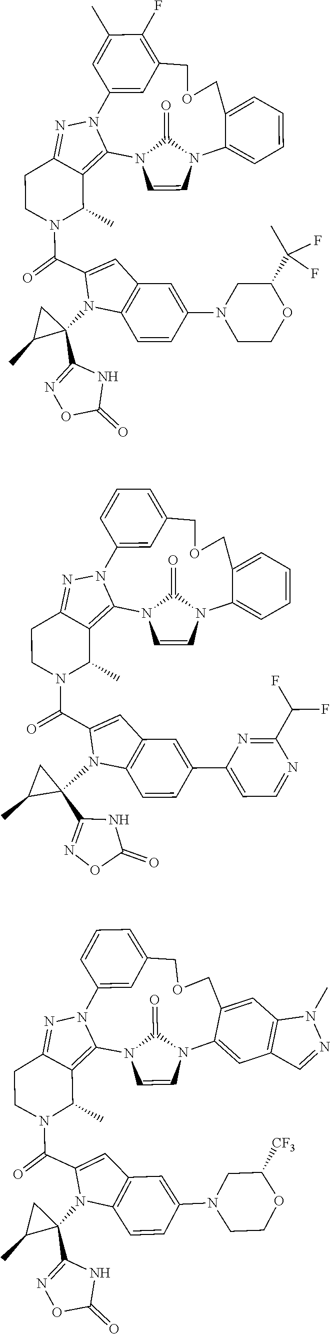

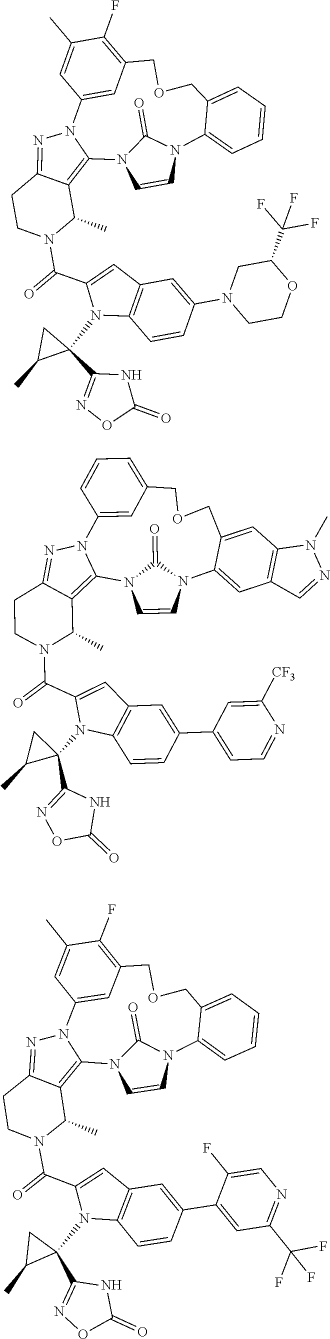

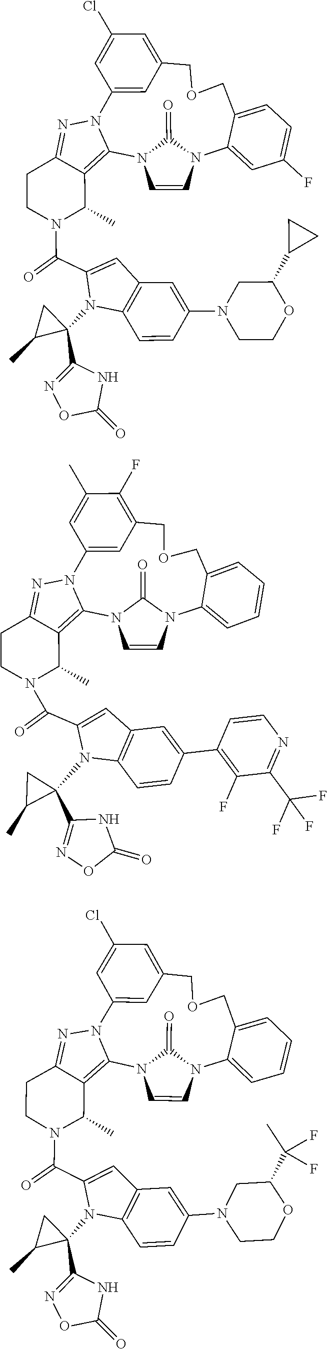

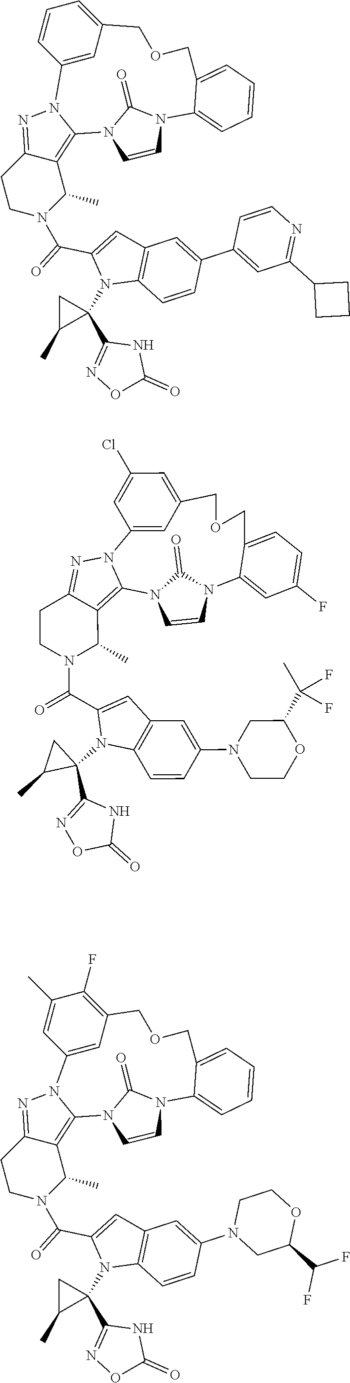

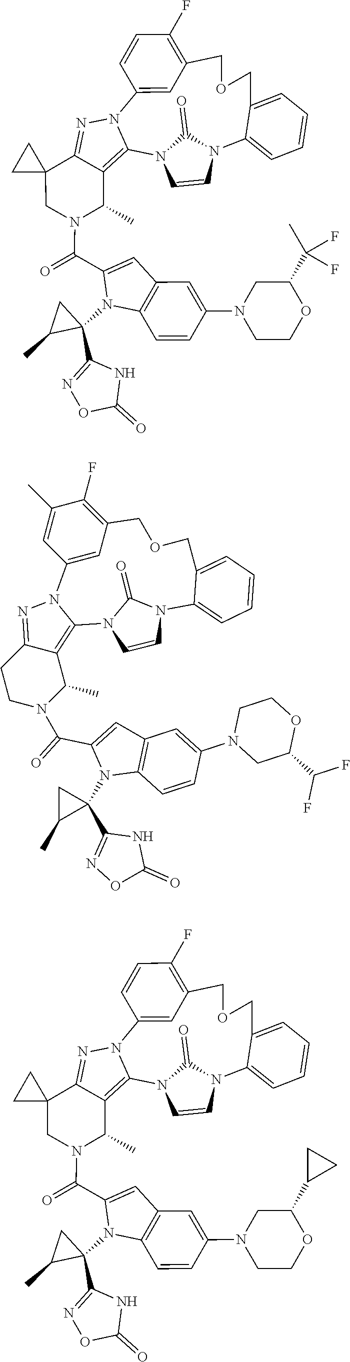

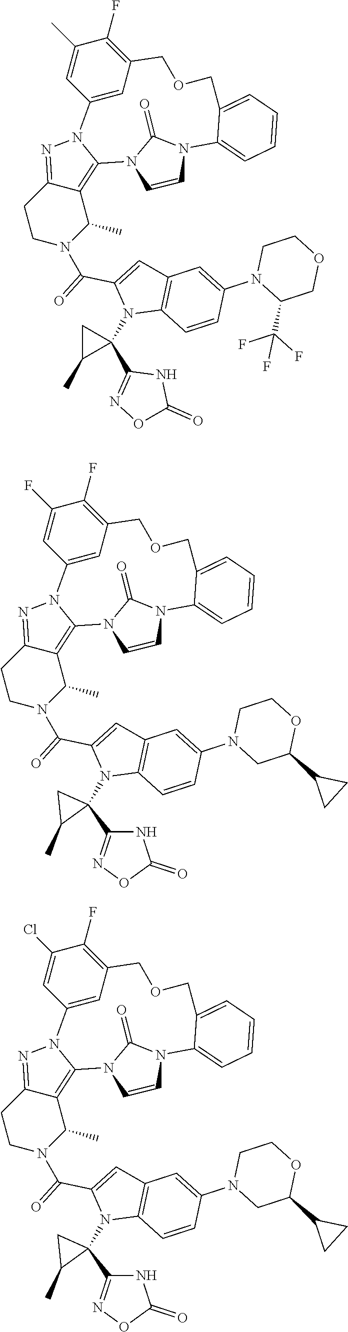

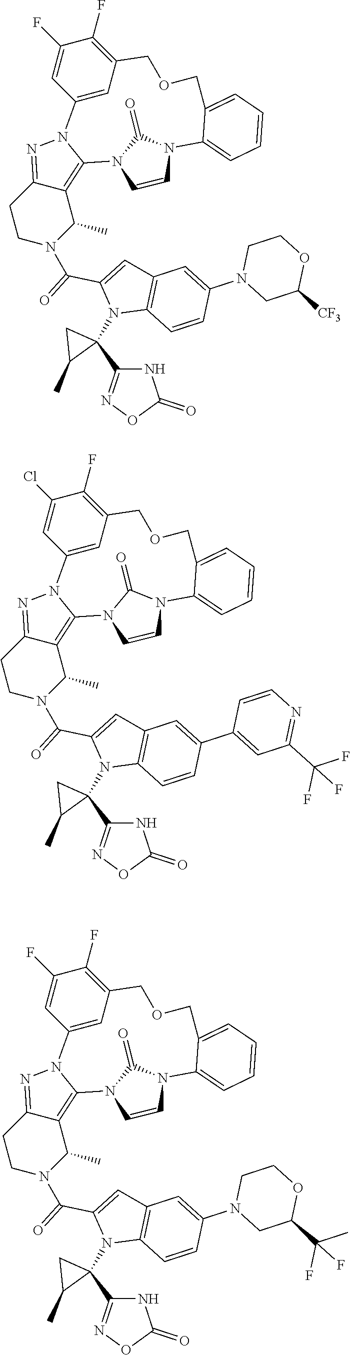

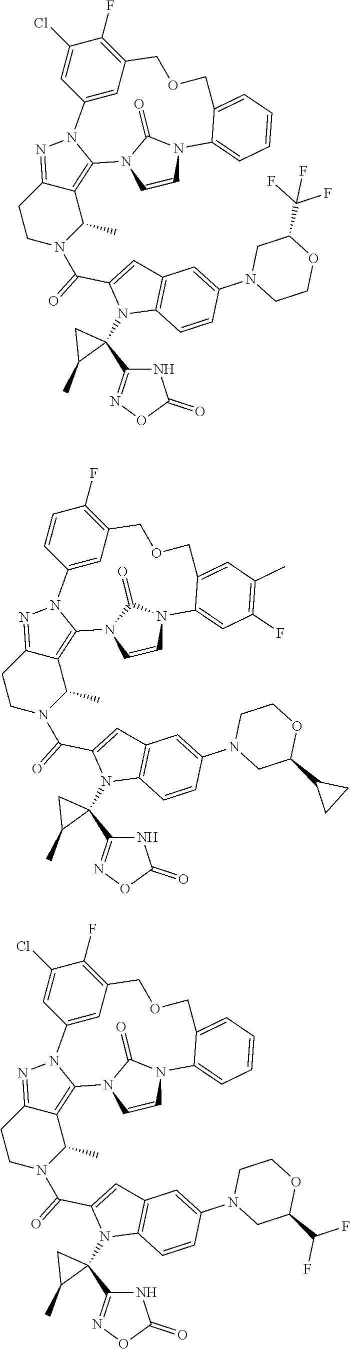

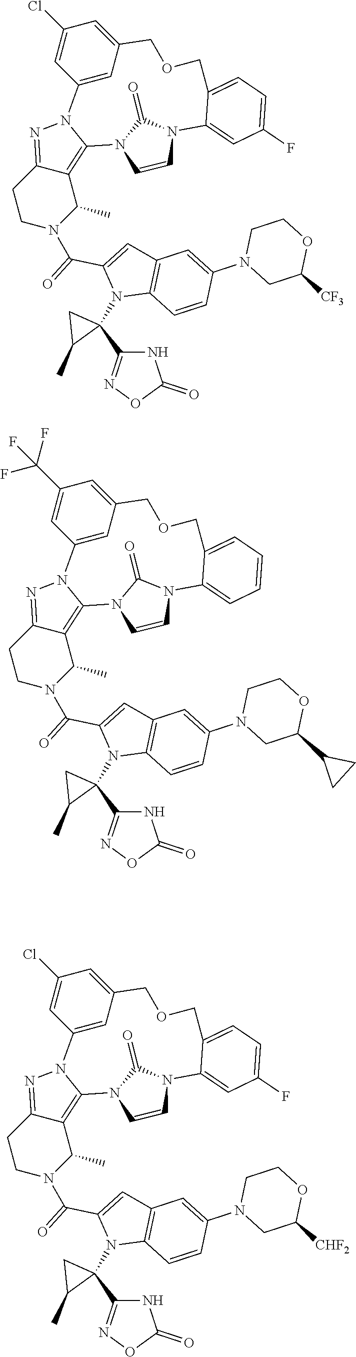

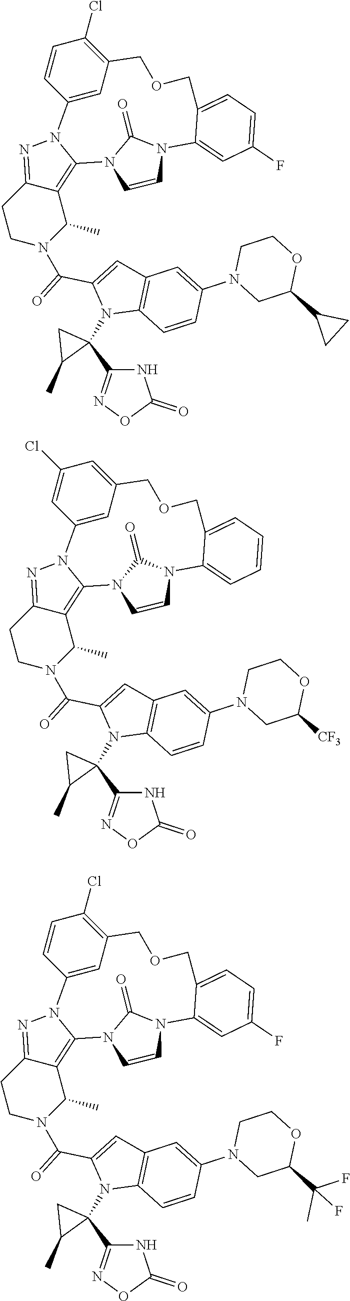

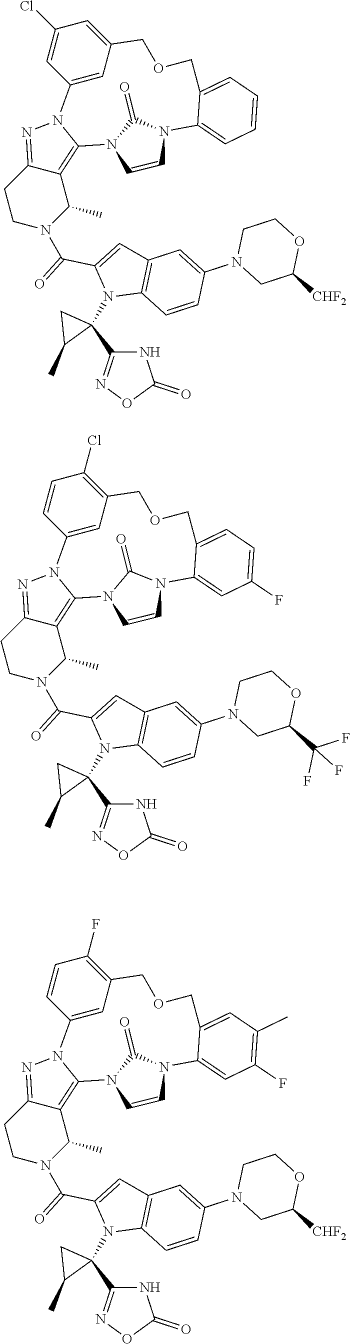

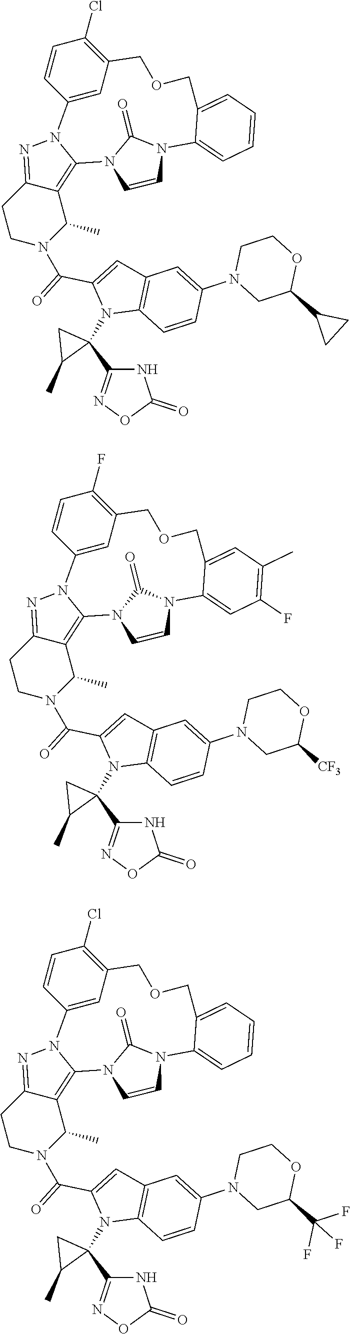

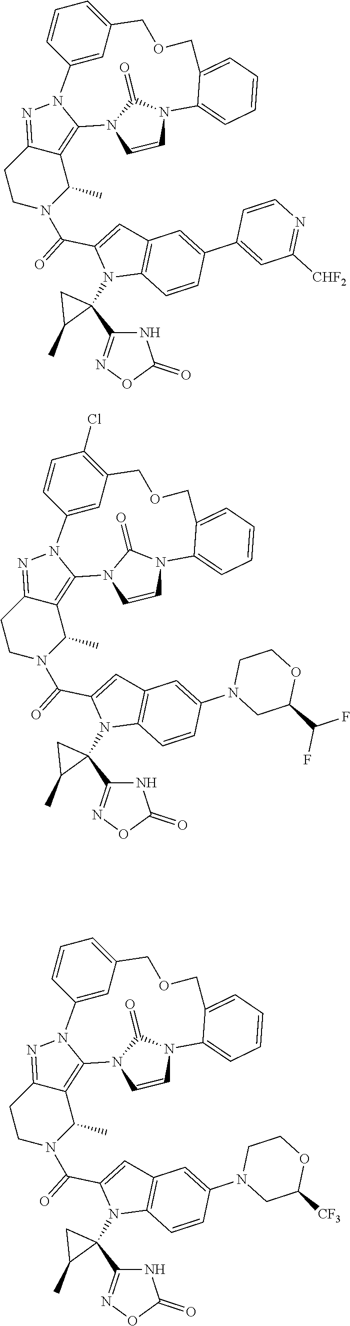

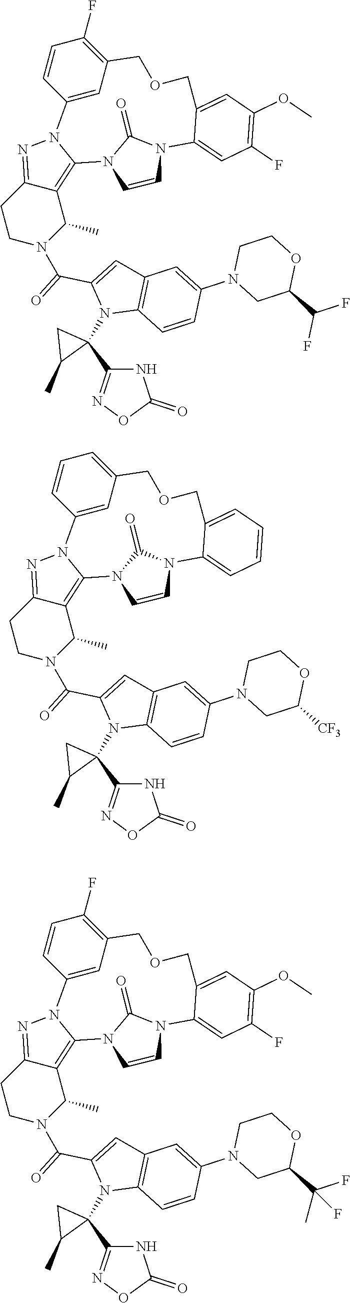

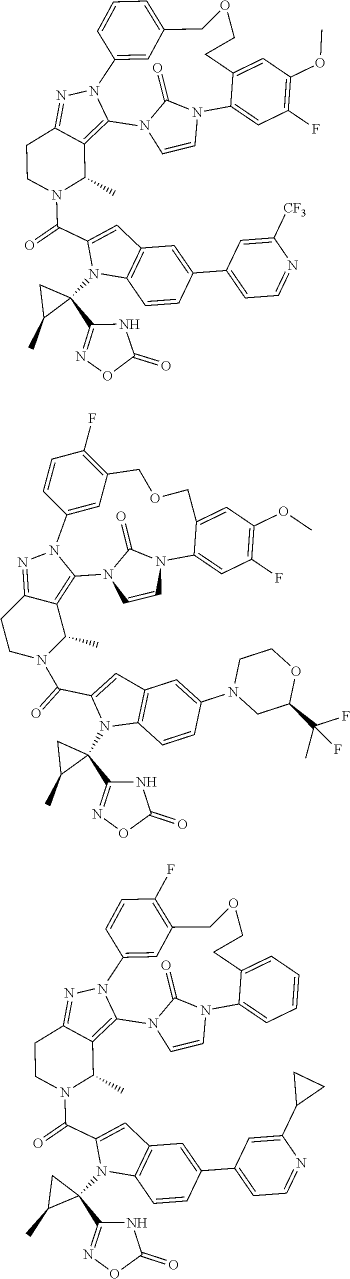

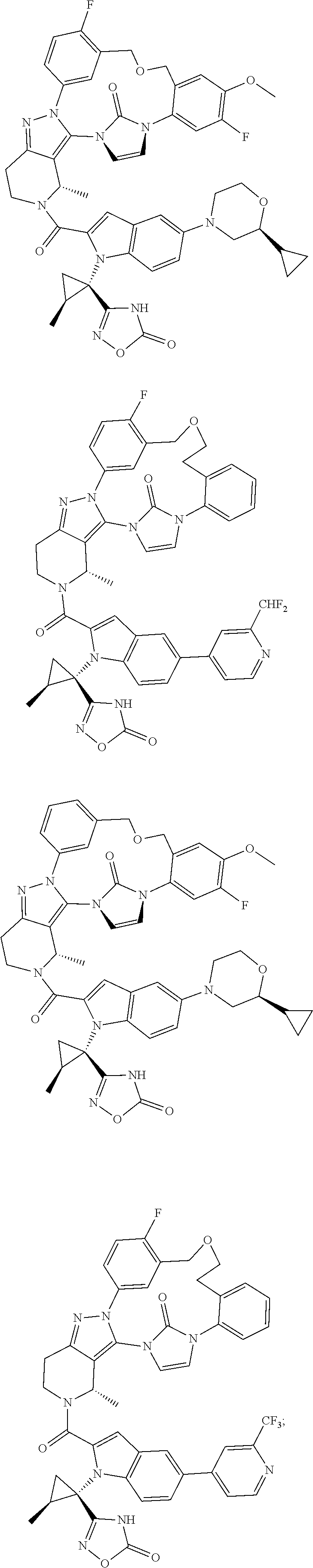

or a pharmaceutically acceptable salt thereof.

In certain aspects, provided herein is a compound selected from:

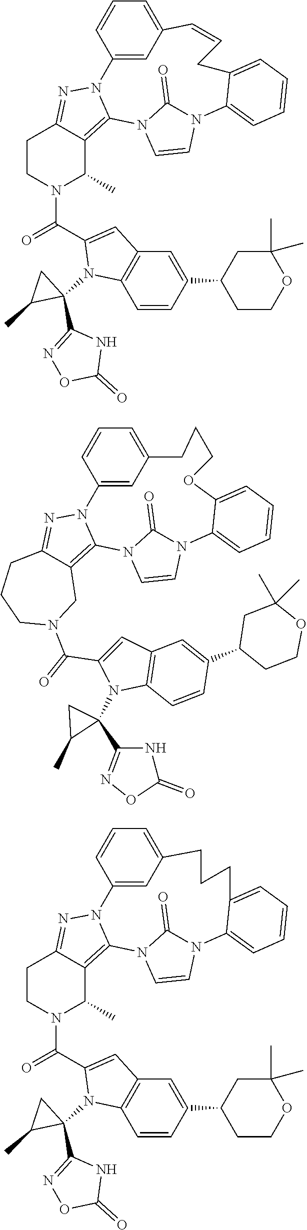

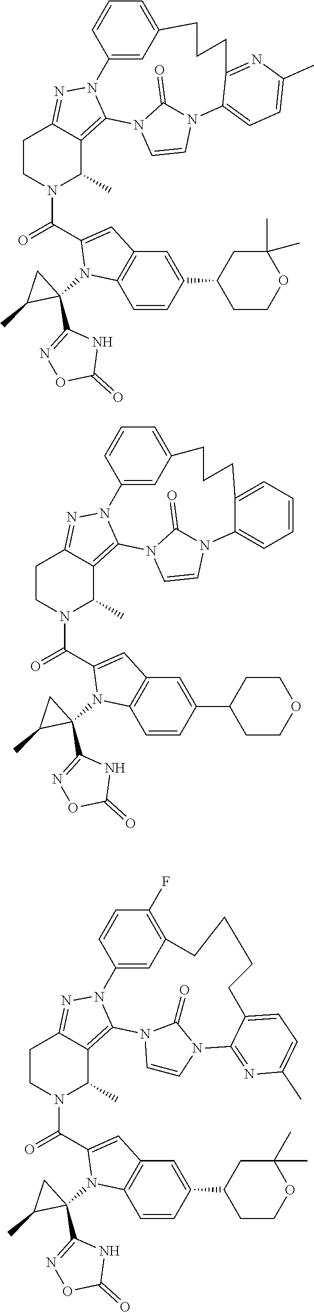

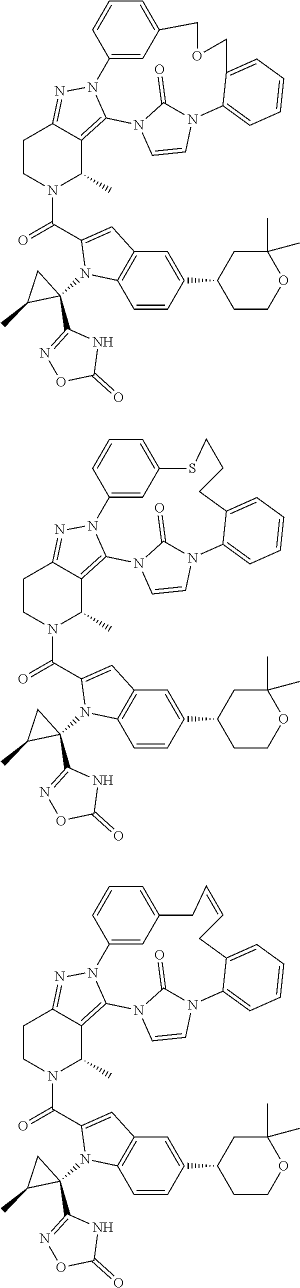

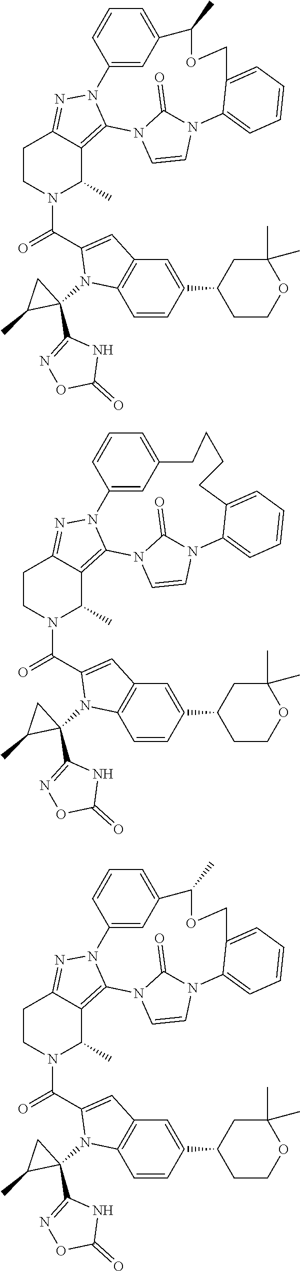

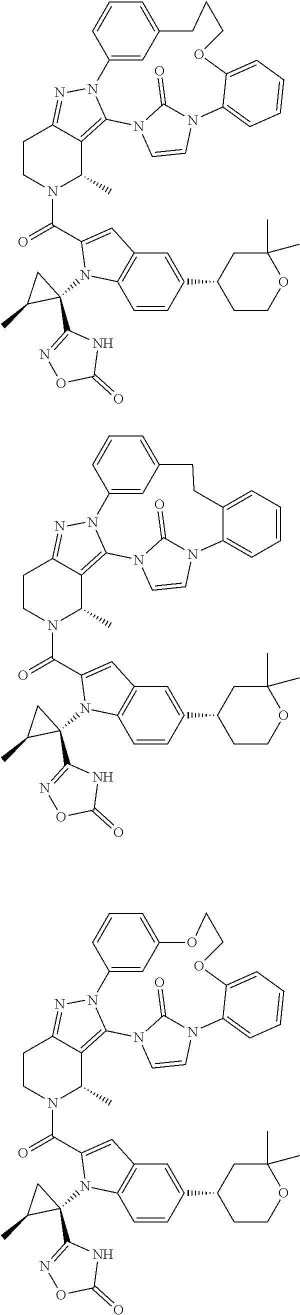

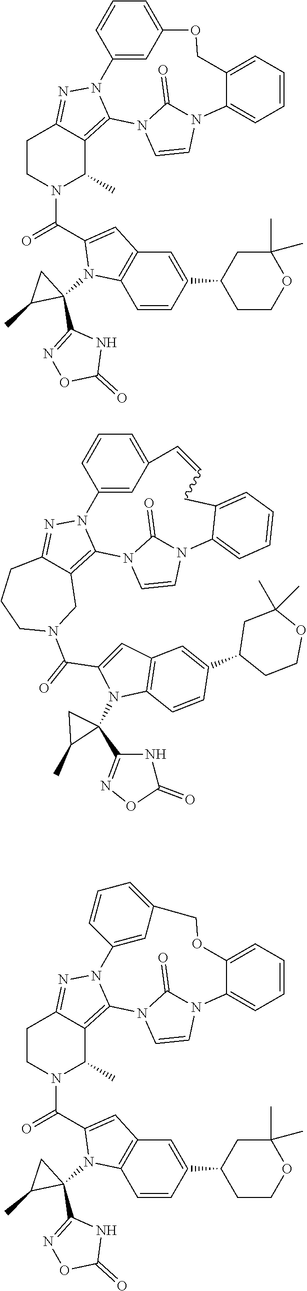

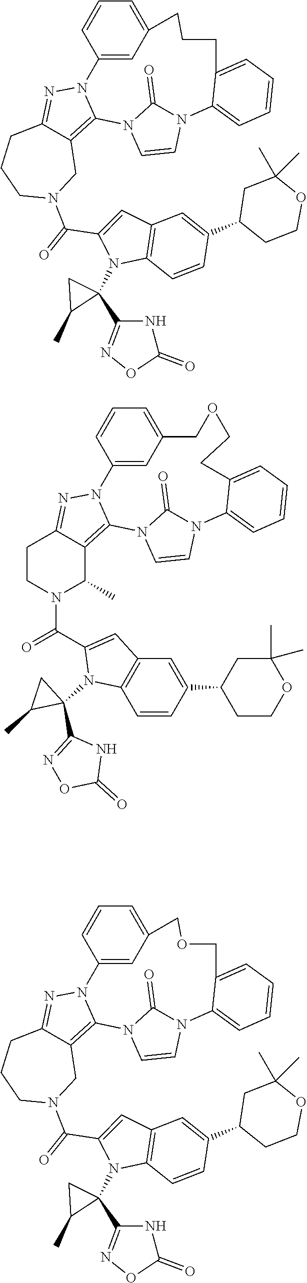

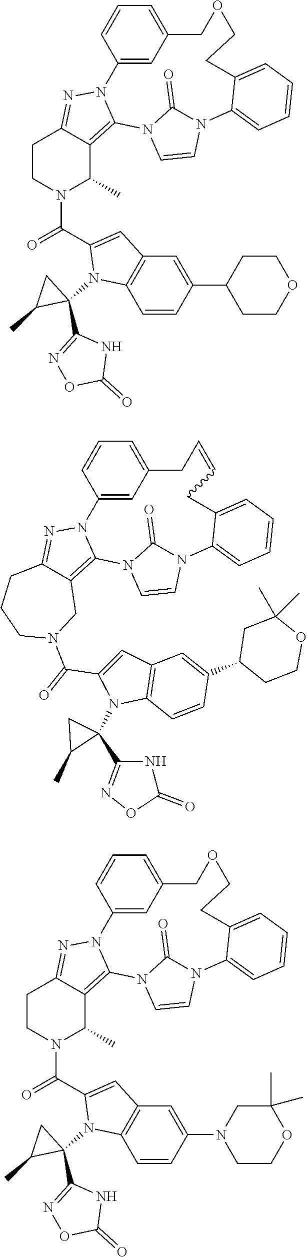

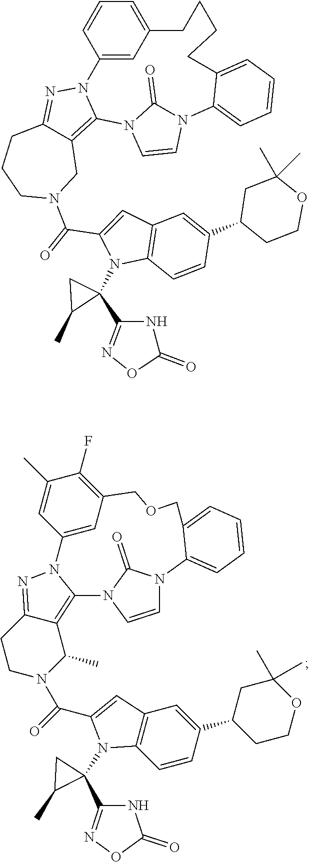 [SMILES: C[C@H]1C[C@]1(c1noc(=O)[nH]1)n1c(C(=O)N2CCCc3nn4c(c3C2)-n2ccn(c2=O)-c2ccccc2CCCCc2cccc-4c2)cc2cc([C@H]3CCOC(C)(C)C3)ccc21 ; Cc1cc2cc(c1F)COCc1ccccc1-n1ccn(c1=O)-c1c3c(nn1-2)CCN(C(=O)c1cc2cc([C@H]4CCOC(C)(C)C4)ccc2n1[C@@]1(c2noc(=O)[nH]2)C[C@@H]1C)[C@H]3C]

or a pharmaceutically acceptable salt thereof.

In certain aspects, provided herein is a compound selected from:

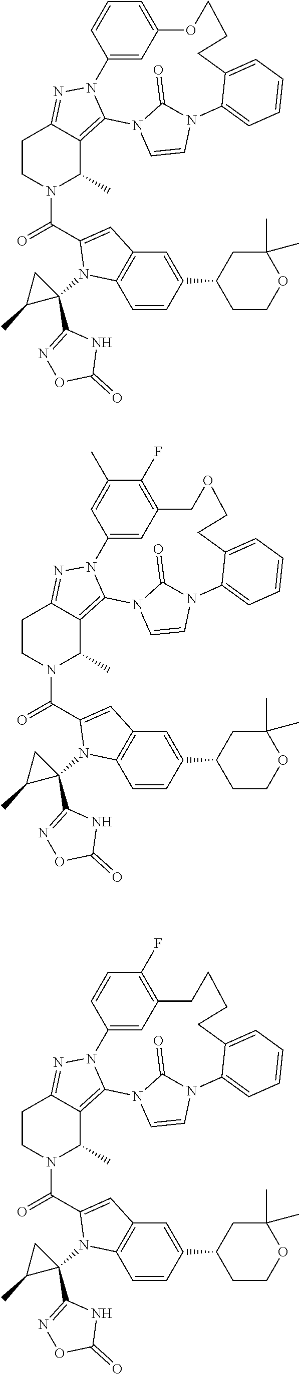

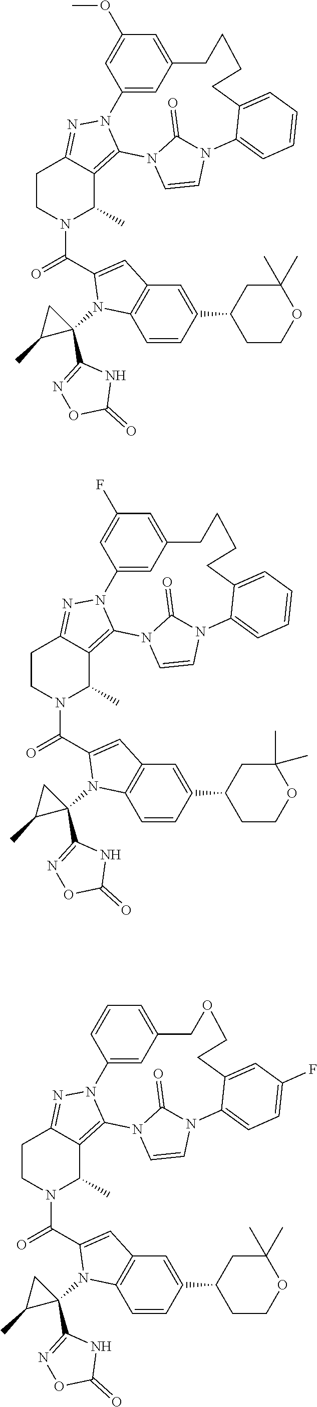

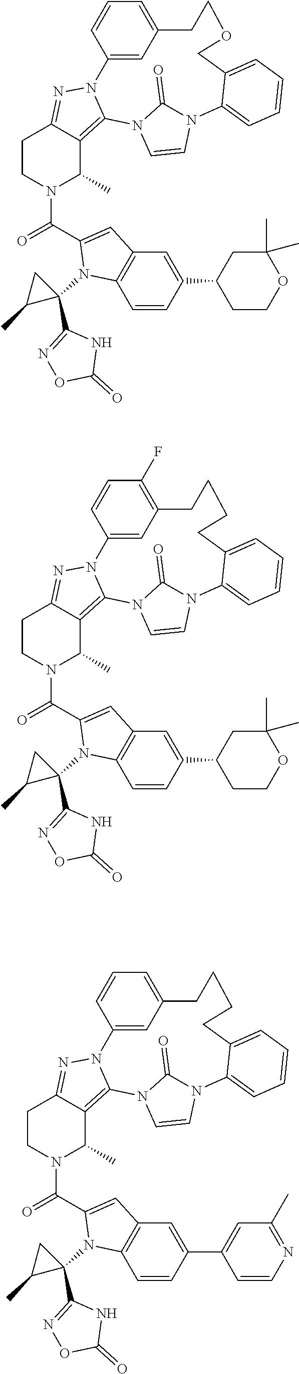

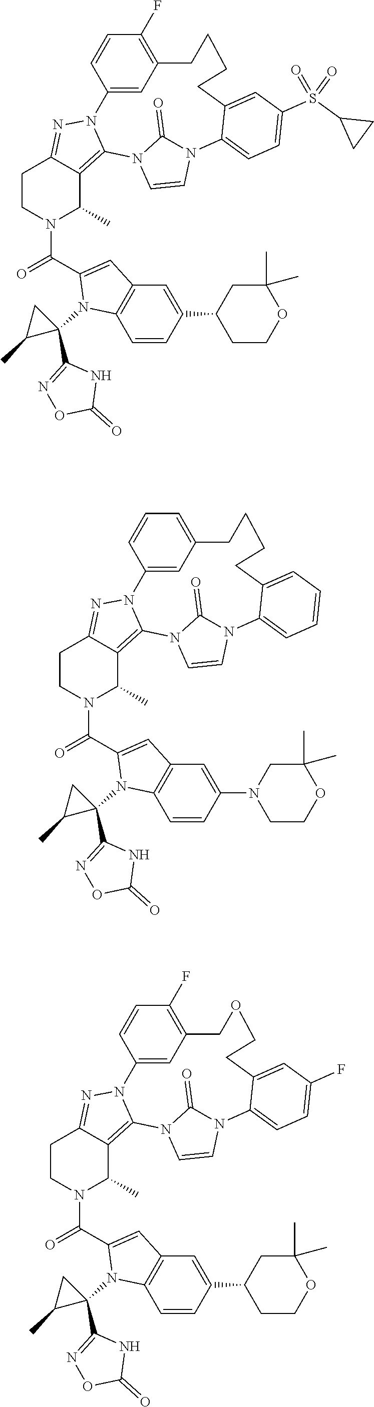

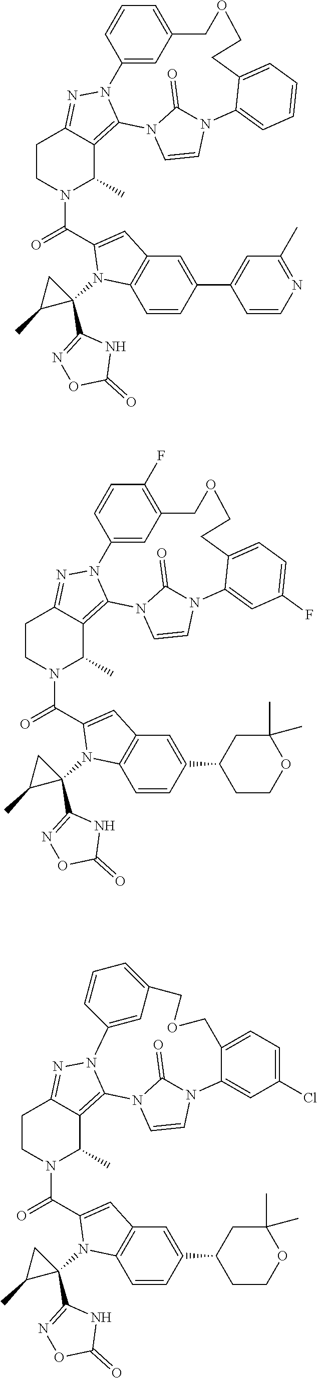

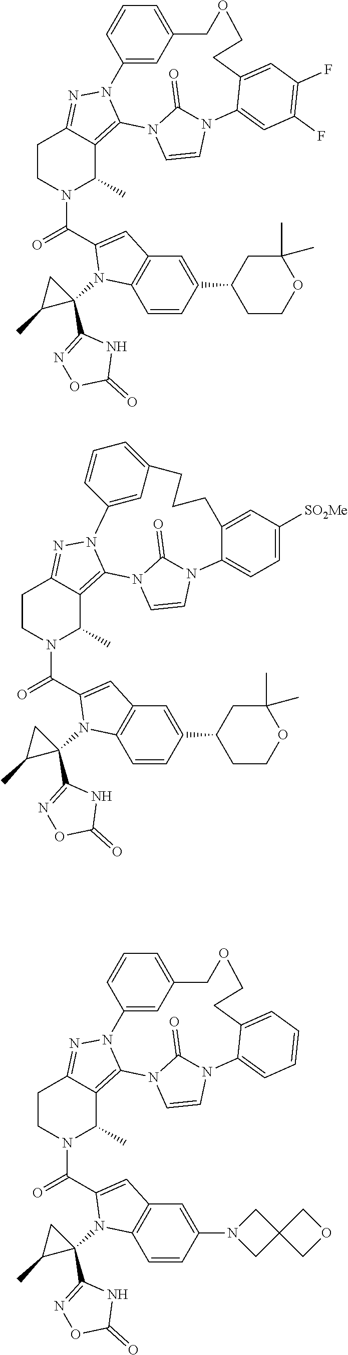

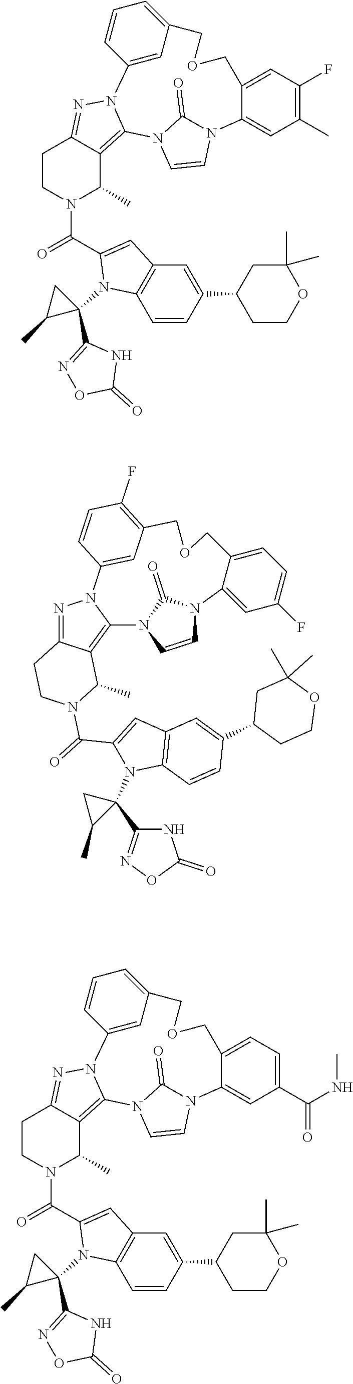

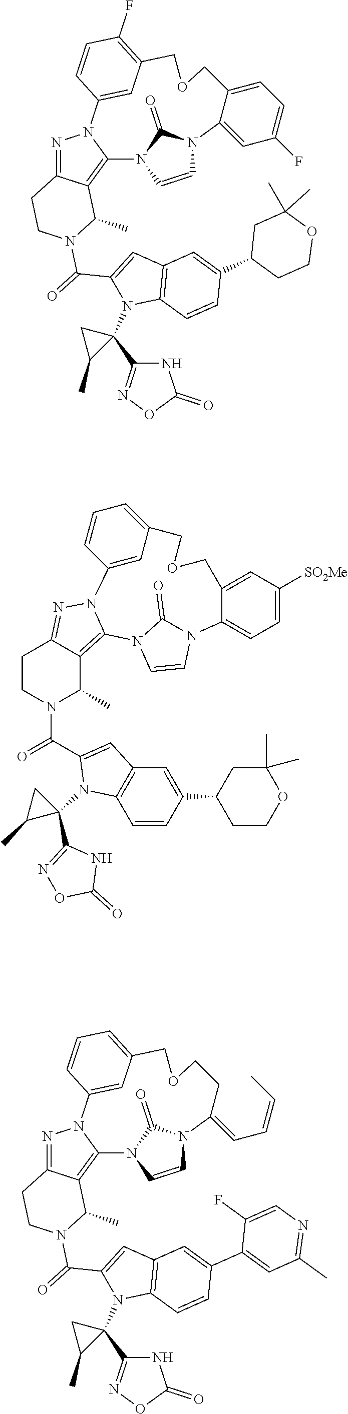

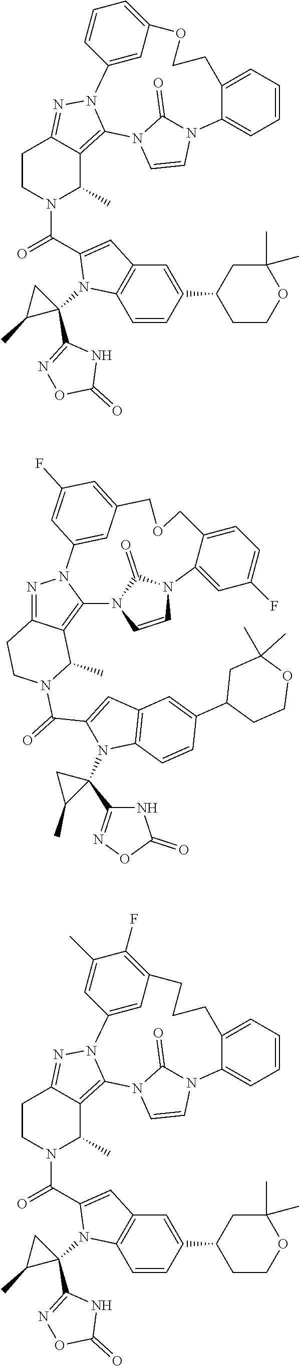

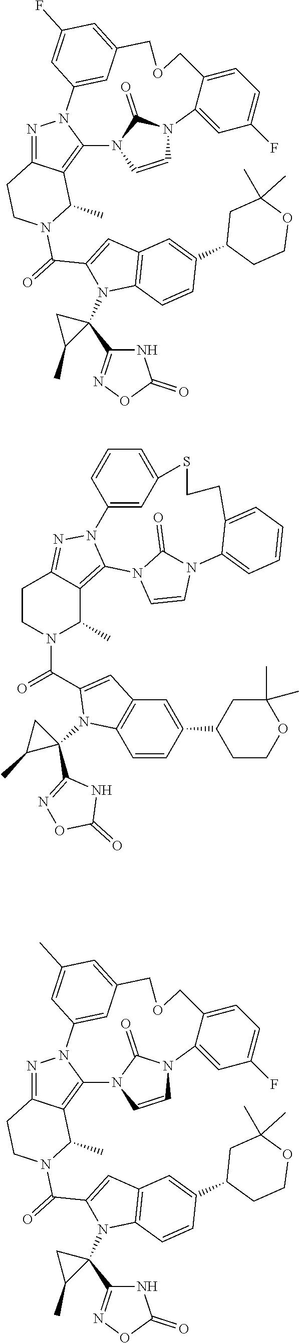

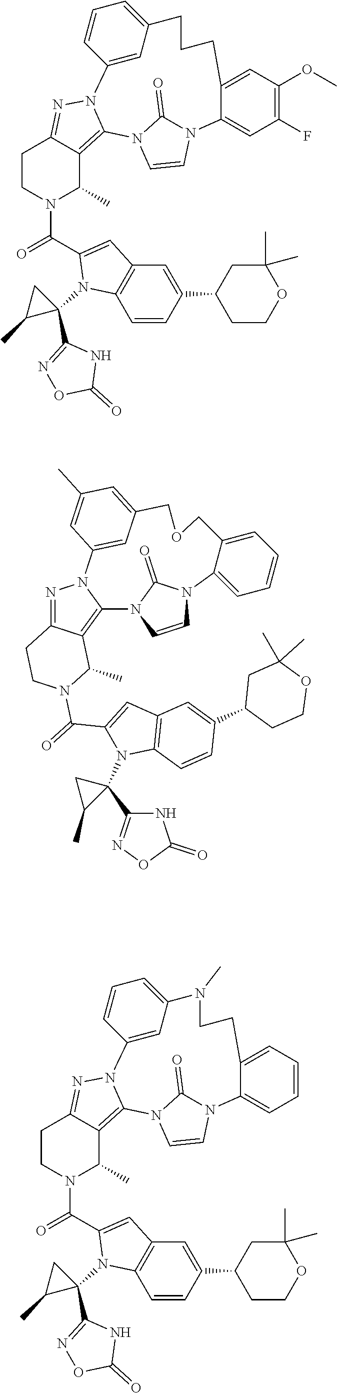

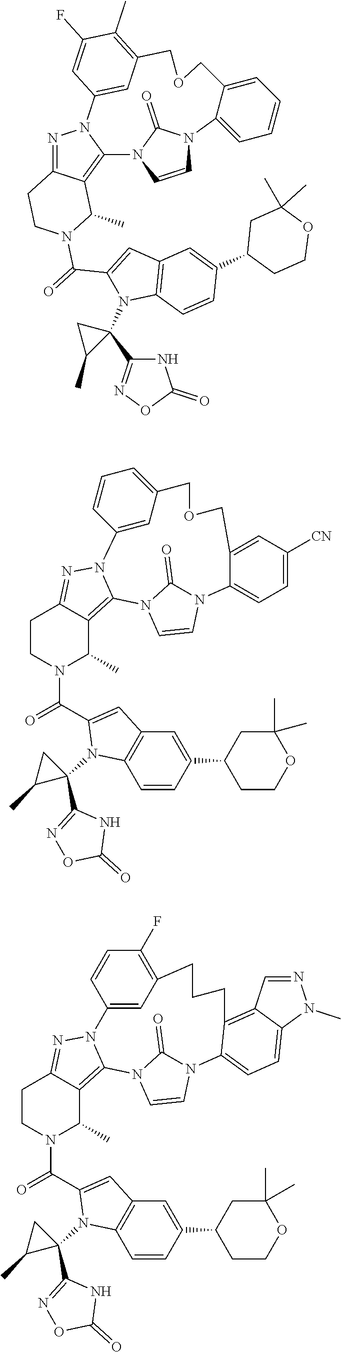

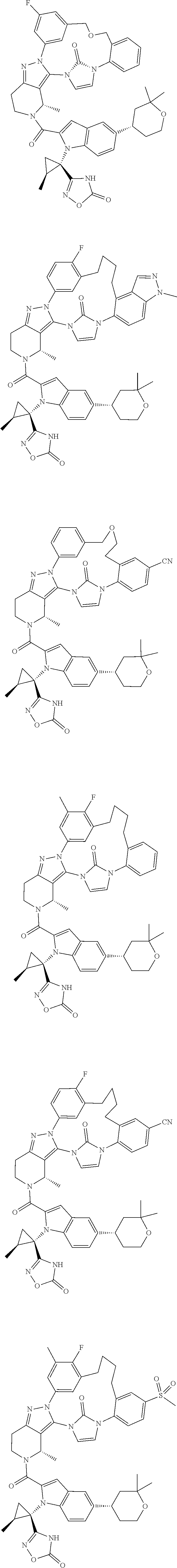

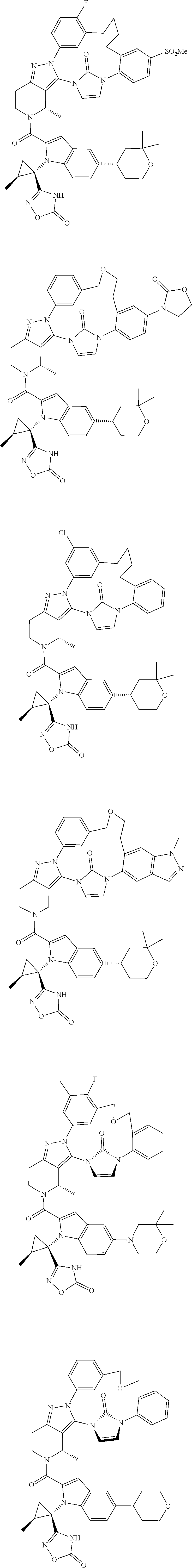

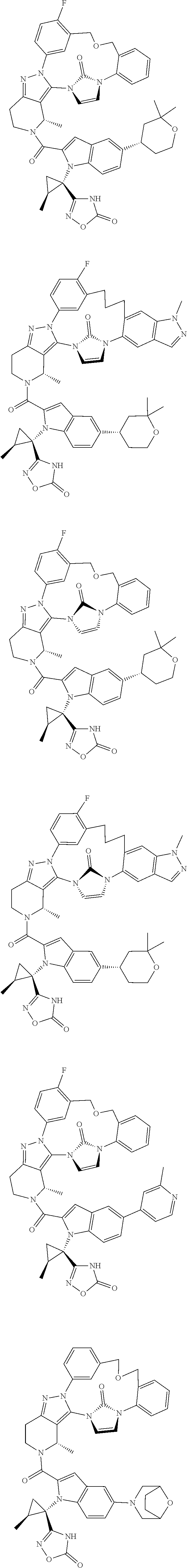

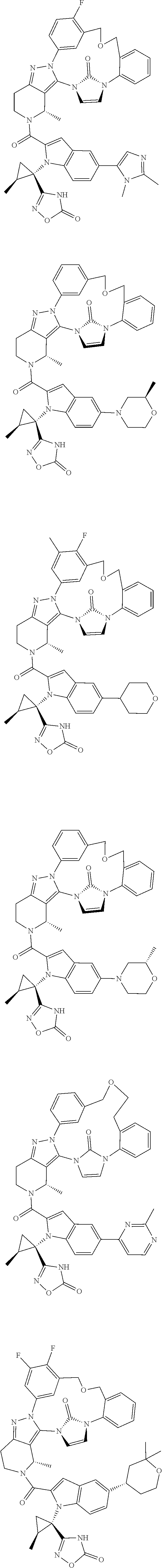

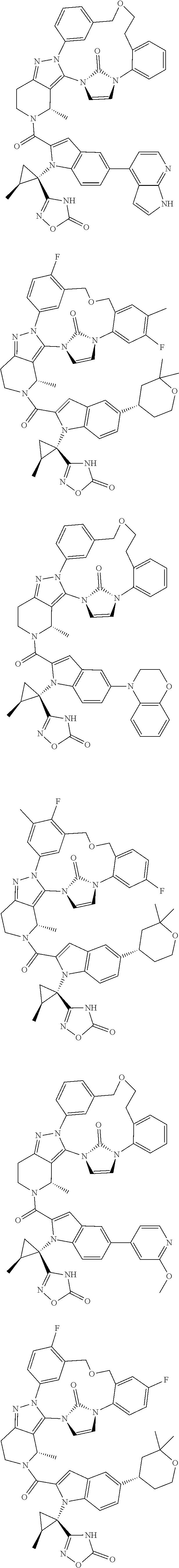

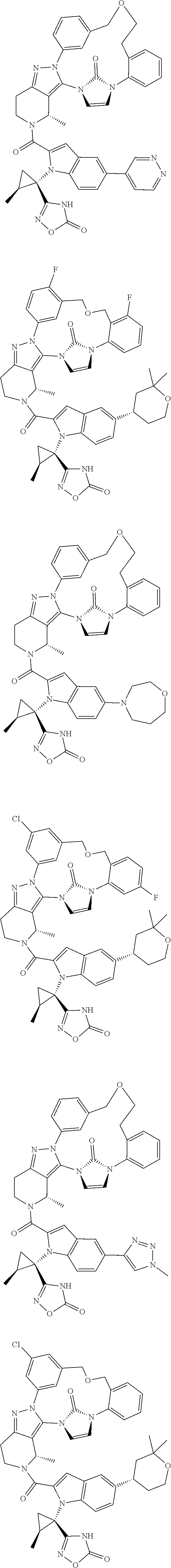

[chemical structure]

[chemical structure]

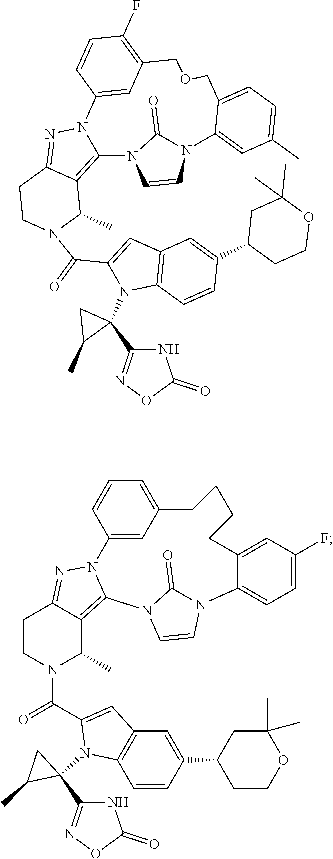

or a pharmaceutically acceptable salt thereof.

In certain aspects, provided herein is a compound selected from:

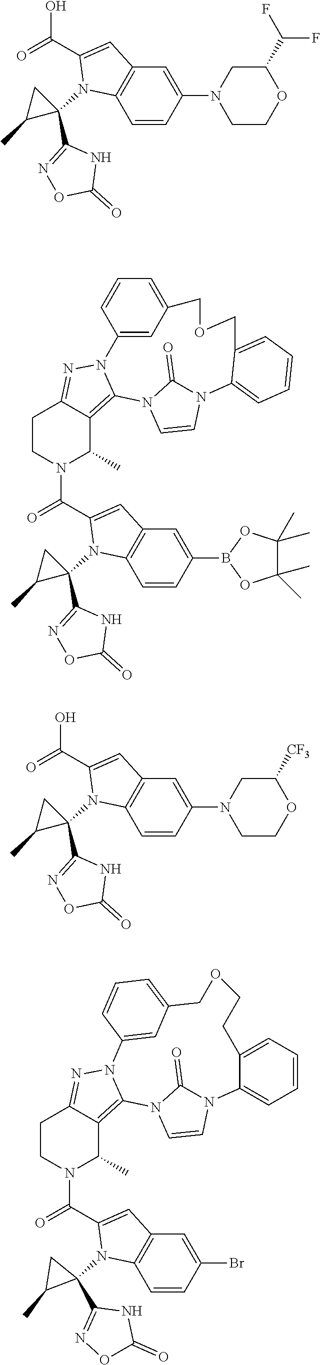

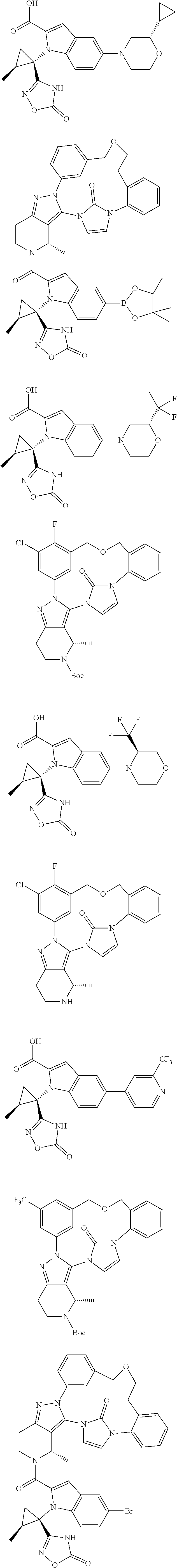

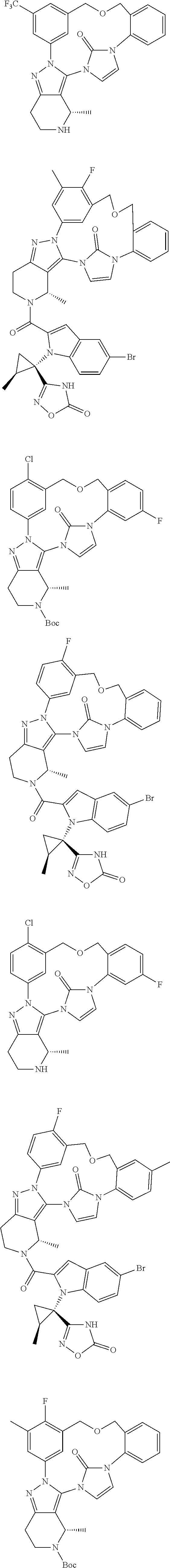

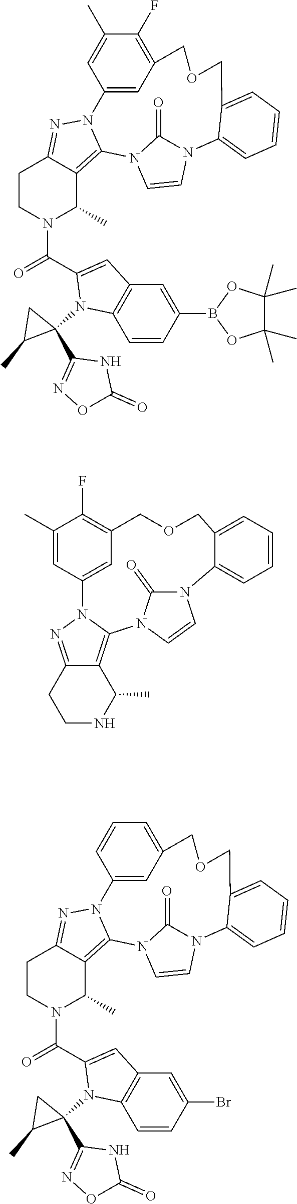

or a salt thereof.

In certain aspects, the present invention provides a compound of formula (I-p):

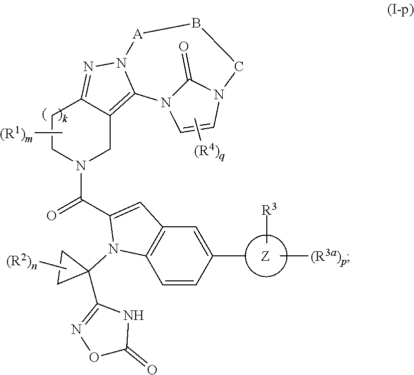

or a pharmaceutically acceptable salt thereof; wherein,
A is optionally substituted arylene, heteroarylene, or heterocycloalkylene;
B is alkylene, alkenylene, —O(alkylene)-, -(alkylene)O—, -(alkylene)O(alkylene)-, —O(alkylene)O—, —O(alkylene)O(alkylene)-, -(alkylene)O(alkylene)O—, —S(alkylene)-, -(alkylene)S—, -(alkylene)S(alkylene)-, —S(alkylene)S—, —S(alkylene)S(alkylene)-, -(alkylene)S(alkylene)S—, —NH(alkylene)-, -(alkylene)NH—, -(alkylene)NH(alkylene)-, —NH(alkylene)NH—, —NH(alkylene)NH(alkylene)-, -(alkylene)NH(alkylene)NH—, or is a bond;
C is optionally substituted arylene, heteroarylene, or heterocycloalkylene;
Ring Z is heteroarylene or heterocycloalkylene;
R¹ is independently for each occurrence alkyl, alkoxy, or halo;
R² is independently for each occurrence alkyl, alkoxy, or halo;
R³ is haloalkyl, unsubstituted cycloalkyl, cycloalkyl substituted with alkyl, cycloalkyl substituted with halo, unsubstituted heteroaryl, heteroaryl substituted with alkyl, heteroaryl substituted with halo, unsubstituted heterocycloalkyl, heterocycloalkyl substituted with alkyl, heterocycloalkyl substituted with halo, haloalkoxy, —NH₂, —NH(alkyl), or —N(alkyl)₂;
R^{3a} is independently for each occurrence alkyl, alkoxy, halo, haloalkyl, optionally substituted cycloalkyl, optionally substituted heteroaryl, optionally substituted heterocycloalkyl, haloalkoxy, —NH₂, —NH(alkyl), or —N(alkyl)₂;
R⁴ is independently for each occurrence alkyl, alkoxy, or halo;
k is an integer selected from 0-2;
m is an integer selected from 0-6;
n is an integer selected from 0-4;
p is an integer selected from 0-4; and
q is an integer selected from 0-2.

In certain aspects, the present invention provides a compound of Formula (I-o):

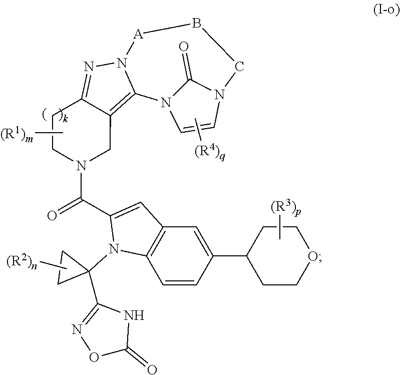

or a pharmaceutically acceptable salt thereof; wherein,
A is optionally substituted arylene, heteroarylene, or heterocycloalkylene;
B is alkylene, alkenylene, —O(alkylene)-, -(alkylene)O—, -(alkylene)O(alkylene)-, —O(alkylene)O—, —O(alkylene)O(alkylene)-, -(alkylene)O(alkylene)O—, —S(alkylene)-, -(alkylene)S—, -(alkylene)S(alkylene)-, —S(alkylene)S—, —S(alkylene)S(alkylene)-, -(alkylene)S(alkylene)S—, —NH(alkylene)-, -(alkylene)NH—, -(alkylene)NH(alkylene)-, —NH(alkylene)NH—, —NH(alkylene)NH(alkylene)-, -(alkylene)NH(alkylene)NH—, or is a bond;
C is optionally substituted arylene, heteroarylene, or heterocycloalkylene;
R¹ is independently for each occurrence alkyl, alkoxy, or halo;
R² is independently for each occurrence alkyl, alkoxy, or halo;
R³ is independently for each occurrence alkyl, alkoxy, or halo;
or two geminal or vicinal occurrences of R³, taken together with the carbon atom or atoms to which they are attached, form a cycloalkyl ring;
R⁴ is independently for each occurrence alkyl, alkoxy, or halo;
k is an integer selected from 0-2;
m is an integer selected from 0-6;
n is an integer selected from 0-4;
p is an integer selected from 0-9; and
q is an integer selected from 0-2.

In certain aspects, the present invention provides a compound of Formula (I-z):

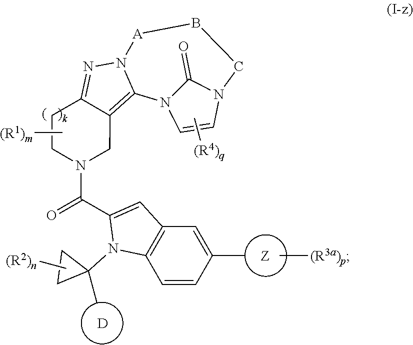

or pharmaceutically acceptable salts thereof; wherein,
A is optionally substituted arylene, heteroarylene, or heterocycloalkylene;
B is -(alkylene)O(alkylene)-, alkylene, haloalkylene, alkenylene, —O(alkylene)-, -(alkylene)O—, —O(alkylene)O—, —O(alkylene)O(alkylene)-, -(alkylene)O(alkylene)O—, —S(alkylene)-, -(alkylene)S—, -(alkylene)S(alkylene)-, —S(alkylene)S—, —S(alkylene)S(alkylene)-, -(alkylene)S(alkylene)S—, —NH(alkylene)-, -(alkylene)NH—, -(alkylene)NH(alkylene)-, —NH(alkylene)NH—, —NH(alkylene)NH(alkylene)-, -(alkylene)NH(alkylene)NH—, —N(alkyl)(alkylene)-, -(alkylene)N(alkyl)-, or is a bond;
C is optionally substituted arylene, heteroarylene, or heterocycloalkylene;
Ring D is

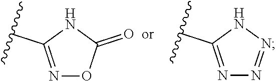

Ring Z is heterocycloalkylene or heteroarylene;
R¹ is independently for each occurrence alkyl, alkoxy, or halo; or two geminal occurrences of R¹ taken together with the carbon atom to which they are attached form an optionally substituted spirocyclic cycloalkyl ring;
R² is independently for each occurrence alkyl, alkoxy, or halo;
R^{3a} is independently for each occurrence alkyl, alkoxy, halo, haloalkyl, optionally substituted cycloalkyl, optionally substituted heteroaryl, optionally substituted heterocycloalkyl, haloalkoxy, —NH₂, —NH(alkyl), or —N(alkyl)₂;
R⁴ is independently for each occurrence alkyl, alkoxy, or halo;
k is an integer selected from 0-2;
m is an integer selected from 0-6;
n is an integer selected from 0-4;
p is an integer selected from 0-4; and
q is an integer selected from 0-2.

### Pharmaceutical Compositions

The invention provides pharmaceutical compositions, each comprising one or more compounds of the invention, or pharmaceutically acceptable salts thereof, and a pharmaceutically acceptable carrier. In certain embodiments, the pharmaceutical composition comprises a compound of the disclosure and a pharmaceutically acceptable carrier. In certain embodiments, the pharmaceutical composition comprises a plurality of compounds of the invention, or pharmaceutically acceptable salts thereof, and a pharmaceutically acceptable carrier.

In certain embodiments, a pharmaceutical composition of the invention further comprises at least one additional pharmaceutically active agent other than a compound of the disclosure. The at least one additional pharmaceutically active agent can be an agent useful in the treatment of a disease or condition that would be benefitted by activation of GLP-1R. For example, the at least one additional pharmaceutically active agent may be a GLP-1R agonist, as described herein.

Pharmaceutical compositions of the invention can be prepared by combining one or more compounds of the invention, or pharmaceutically acceptable salts thereof, with a pharmaceutically acceptable carrier and, optionally, one or more additional pharmaceutically active agents.

### Methods of Use

The present invention provides compounds, and pharmaceutically acceptable salts thereof, that are useful for treating or preventing a disease or condition whose treatment would benefit from GLP-1R activation.

In certain embodiments, the compounds described herein are useful for modulating the activity of the glucagon-like peptide-I (GLP-1) receptor. In certain embodiments, the compounds described herein are agonists of the glucagon-like peptide-1 (GLP-1) receptor. In certain embodiments, the compounds described herein activate, or induce activity of, the glucagon-like peptide-I (GLP-1) receptor.

In certain aspects, the invention provides a method of inducing GLP-1R activity in a subject in need thereof, comprising administering to the subject a therapeutically effective amount of a compound of the disclosure (e.g., a compound of formula (I) or a pharmaceutically acceptable salt thereof). In certain aspects, the invention provides a method of inducing GLP-1R activity in a subject in need thereof, comprising administering to the subject an amount of a compound of the disclosure (e.g., a compound of formula (I) or a pharmaceutically acceptable salt thereof, or a pharmaceutical composition comprising the same). In certain embodiments, the amount is an effective amount.

In certain aspects, the invention provides a compound of the disclosure, or a pharmaceutically acceptable salt thereof, for use as a medicament.

The present invention also provides a method of treating a GLP-1-associated disease, disorder, or condition in a subject in need thereof, the method comprising administering to the subject a therapeutically effective amount of a compound of the disclosure, or a pharmaceutically acceptable salt thereof, or a pharmaceutical composition comprising the same. In some aspects, the present invention provides a method of treating a GLP-1-associated disease, disorder, or condition in a subject in need thereof, the method comprising administering to the subject an amount of a compound of the disclosure, or a pharmaceutically acceptable salt thereof, or a pharmaceutical composition comprising the same. In certain embodiments, the amount is a therapeutically effective amount.

In certain embodiments, the GLP-1-associated disease, disorder, or condition is type I diabetes mellitus, type 2 diabetes mellitus, early onset type 2 diabetes mellitus, idiopathic type I diabetes mellitus (Type Ib), youth onset atypical diabetes (YOAD), maturity onset diabetes of the young (MODY), latent autoimmune diabetes in adults (LADA), obesity, weight gain from use of other agents, idiopathic intracranial hypertension, Wolfram syndrome, gout, excessive sugar craving, hypertriglyceridemia, dyslipidemia, hyperlipidemia, malnutrition-related diabetes, gestational diabetes, kidney disease, adipocyte dysfunction, sleep apnea, obstructive sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, hyperosmolar hyperglycemic state, post-prandial lipemia, metabolic acidosis, diabetic ketoacidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, anginapectoris, diabetic nephropathy, impaired glucose tolerance, neuropathy, diabetic neuropathy, neuropathy of eight cranial nerve, neuropathic pain, diabetic retinopathy, macular degeneration, cataract, glomerulosclerosis, arthritis, primary and secondary osteoarthritis, psoriatic arthritis, rheumatoid arthritis, osteoporosis, treatment of addiction, cocaine dependence, bipolar disorder, major depressive disorder, cognitive dysfunction in major depressive disorder, persistent depressive disorder (dysthymia), skin and connective tissue disorders, foot ulcerations, foot ulcers, presure ulcers, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's disease, Alzheimer's disease, Huntington's disease, impaired cognition, schizophrenia, Polycystic Ovary Syndrome (PCOS), peri- and post-menopausal symptom regulation, peri- and post-menopausal hormone regulation, gambling disorder, hypersexuality, adrenaline addiction, tobacco-use disorder, substance use disorder, metabolic dysfunction-associated steatotic liver disease (MASLD), spinal stenosis, degenerative disc disease, fractures, major and mild neurocognitive disorder, multiple sclerosis, Addison's disease, celiac disease, Grave's disease, Hashimoto's thyroiditis, myasthenia gravis, pernicious anemia, Sjögren's syndrome, systemic lupus erythematosus, cancer, cholecystitis, coronary artery disease, cardiac fibrosis, atrial fibrillation, arrythmia, renal fibrosis, asthma, restrictive lung disease, erectile dysfunction, infertility, glaucoma, macular edema, diabetic papillopathy, nyctalopia, sensorineural hearing loss, auditory neuropathy spectrum disorder, auditory processing disorder, malignant otitis externa, meniere's disease, gastroparesis, gastroesophageal reflux disease, hepatic fibrosis, chronic and acute pancreatitis, infection-associated chronic condition (long COVID), cellulitis (bacterial and fungal), periodontal disease, HIV lipohypertrophy, necrobiosis lipoidica, acanthosis nigricans, diabetic dermopathy, scleroderma-type skin changes, scleroderma diabetocorum, bullosis diabeticorum, ichthyosiform changes of the shins, xerosis, acquired perforating dermatosis, eruptive xanthomas, acrochordons, diabetes-associated pruritis, Huntley's papules, keratosis pilaris, pigmented purpuric dermatosis, palmar erythema, periungual telangiectasias, rubeosis faciei, onychocryptosis, generalized granuloma annulare, vitiligo, hidradenitis supparativa, glucagonoma, all-cause mortality, sedentary lifestyle, body dysmorphic disorder, inferiority complex, or any combination thereof.

In certain embodiments, the GLP-1-associated disease, disorder, or condition is obesity.

In certain embodiments, the GLP-1-associated disease, disorder, or condition is diabetes.

In certain embodiments, the GLP-1-associated disease, disorder, or condition is cancer.

In certain embodiments, the cancer is endometrial cancer, breast cancer, colorectal cancer, prostate cancer, liver cancer, gallbladder cancer, kidney cancer, ovarian cancer, esophageal cancer, meningioma cancer, stomach cancer, or pancreatic cancer.

The compounds of the invention are useful in treating any disease or condition whose treatment would benefit from GLP-1R activation, meaning that in such disease or condition it would be desirable to increase GLP-1R activity. For example, it may be desirable to increase GLP-1R activity in the setting of inappropriate inactivation of GLP-1R.

In any of the foregoing, an additional pharmaceutically active agent other than a compound of the disclosure may also be administered to the subject.

### Formulations, Routes of Administration, and Dosing

The compounds of the invention, and pharmaceutically acceptable salts thereof, either alone or as a component of a pharmaceutical composition, can be administered to a mammalian host, such as a human patient, in a variety of forms adapted to the chosen route of administration, e.g., orally or parenterally, by intravenous, intraperitoneal, intramuscular, topical, or subcutaneous routes. Additional routes of administration are also contemplated by the invention.

Thus, the present compounds or pharmaceutically acceptable salts thereof may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound, or a pharmaceutically acceptable salt thereof, may be combined with one or more pharmaceutically acceptable carriers and used in the form of ingestible tablets, buccal tablets, troches, capsules, elixirs, suspensions, syrups, wafers, and the like. In some embodiments, such compositions and preparations contain at least 0.1% by weight of active compound, or a pharmaceutically acceptable salt thereof. The percentage of the active compound, or a pharmaceutically acceptable salt thereof, in such compositions and preparations may, of course, be varied and may conveniently be between about 2% to about 60% of the weight of a given unit dosage form. In some embodiments, the amount of active compound, or a pharmaceutically acceptable salt thereof, in such compositions is a therapeutically effective amount.

The tablets, troches, pills, capsules, and the like may also contain the following diluents and carriers: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, or a pharmaceutically acceptable salt thereof, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound, or a pharmaceutically acceptable salt thereof, may be incorporated into sustained-release preparations and devices.

The active compound, or a pharmaceutically acceptable salt thereof, may also be administered intravenously or intraperitoneally by infusion or injection. Solutions of the active compound, or a pharmaceutically acceptable salt thereof, can be prepared in water or physiologically acceptable aqueous solution, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active compound, or a pharmaceutically acceptable salt thereof, which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound, or a pharmaceutically acceptable salt thereof, in the required amount in the appropriate solvent with various of the other ingredients enumerated above, as required, followed by filter sterilization. In the case of sterile powders for the preparation of sterile injectable solutions, methods of preparation can include vacuum drying and the freeze drying techniques, which yield a powder of the active compound, or a pharmaceutically acceptable salt thereof, plus any additional desired ingredient present in the previously sterile-filtered solutions.

For topical administration, the active compound, or a pharmaceutically acceptable salt thereof, may be applied in pure form, i.e., when they are liquids. However, it will generally be desirable to administer them to the skin as compositions or formulations, in combination with a pharmaceutically acceptable carrier suitable for dermatologic use, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the active compound, or pharmaceutically acceptable salt thereof, can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the invention, or pharmaceutically acceptable salts thereof, to the skin are known in the art; for example, see Jacquet et al. (U.S. Pat. No. 4,608,392; incorporated herein by reference), Geria (U.S. Pat. No. 4,992,478; incorporated herein by reference), Smith et al. (U.S. Pat. No. 4,559,157; incorporated herein by reference), and Wortzman (U.S. Pat. No. 4,820,508; incorporated herein by reference).

Useful dosages of the active compound, or a pharmaceutically acceptable salt thereof, can be determined, at least initially, by comparing their in vitro activity and in vivo activity in animal models. Methods for the extrapolation of effective dosages in mice, and other animals, to humans are known in the art; for example, see U.S. Pat. No. 4,938,949 (incorporated herein by reference).

The amount of the active compound, or a pharmaceutically acceptable salt thereof, required for use in treatment will vary not only with the particular compound or salt selected but also with the route of administration, the nature of the condition being treated, and the age and condition of the patient and will be ultimately at the discretion of the attendant physician or clinician.

In general, however, a suitable dose will be in the range of from about 0.5 to about 100 mg/kg body weight of the recipient per day, e.g., from about 3 to about 90 mg/kg of body weight per day, from about 6 to about 75 mg per kilogram of body weight per day, from about of 10 to about 60 mg/kg of body weight per day, or from about 15 to about 50 mg/kg of body weight per day.

An active compound, or a pharmaceutically acceptable salt thereof, can be conveniently formulated in unit dosage form; for example, containing 5 to 1000 mg, 10 to 750 mg, or 50 to 500 mg of active compound, or a pharmaceutically acceptable salt thereof, per unit dosage form. In certain embodiments, the invention provides a composition comprising an active compound, or pharmaceutically acceptable salt thereof, formulated in such a unit dosage form. The desired dose may conveniently be presented in a single dose or as divided doses to be administered at appropriate intervals, for example, as two, three, four or more sub-doses per day. The sub-dose itself may be further divided, e.g., into a number of discrete loosely spaced administrations.

An active compound, or a pharmaceutically acceptable salt thereof, can also be administered in combination with other therapeutic agents, for example, other agents that are useful for treating or preventing a disease or condition whose treatment would benefit from GLP-1R activation.

Other delivery systems can include time-release, delayed release, or sustained release delivery systems such as are well-known in the art. Such systems can avoid repeated administrations of the active compound, or a pharmaceutically acceptable salt thereof, increasing convenience to the subject and the physician. Many types of release delivery systems are available and known to those of ordinary skill in the art. Use of a long-term sustained release implant may be desirable. Long-term release, as used herein, means that the delivery system or is implant constructed and arranged to deliver therapeutic levels of the active compound, or a pharmaceutically acceptable salt thereof, for at least 30 days, and preferably 60 days.

In certain embodiments, an active compound, or pharmaceutically acceptable salt thereof, is formulated for intraocular administration, for example direct injection or insertion within or in association with an intraocular medical device.

An active compound, or a pharmaceutically acceptable salt thereof, may be formulated for depositing into a medical device, which may include any of a variety of conventional grafts, stents, including stent grafts, catheters, balloons, baskets, or other device that can be deployed or permanently implanted within a body lumen. As a particular example, it would be desirable to have devices and methods which can deliver compounds of the invention, or pharmaceutically acceptable salts thereof, to the region of a body which has been treated by interventional technique.

In exemplary embodiments, an active compound, or a pharmaceutically acceptable salt thereof, may be deposited within a medical device, such as a stent, and delivered to the treatment site for treatment of a portion of the body.

Stents have been used as delivery vehicles for therapeutic agents (i.e., drugs). Intravascular stents are generally permanently implanted in coronary or peripheral vessels. Stent designs include those of U.S. Pat. No. 4,733,655 (Palmaz), U.S. Pat. No. 4,800,882 (Gianturco), or U.S. Pat. No. 4,886,062 (Wiktor). Such designs include both metal and polymeric stents, as well as self-expanding and balloon-expandable stents. Stents may also be used to deliver a drug at the site of contact with the vasculature, as disclosed in U.S. Pat. No. 5,102,417 (Palmaz), U.S. Pat. No. 5,419,760 (Narciso, Jr.), U.S. Pat. No. 5,429,634 (Narciso, Jr.), and in International Patent Application Nos. WO 91/12779 (Medtronic, Inc.) and WO 90/13332 (Cedars-Sanai Medical Center), for example.

The term “deposited” means that the active compound, or a pharmaceutically acceptable salt thereof, is coated, adsorbed, placed, or otherwise incorporated into the device by methods known in the art. For example, the active compound, or a pharmaceutically acceptable salt thereof, may be embedded and released from within (“matrix type”) or surrounded by and released through (“reservoir type”) polymer materials that coat or span the medical device. In the latter example, the active compound, or a pharmaceutically acceptable salt thereof, may be entrapped within the polymer materials or coupled to the polymer materials using one or more the techniques for generating such materials known in the art. In other formulations, the active compound, or a pharmaceutically acceptable salt thereof, may be linked to the surface of the medical device without the need for a coating, for example by means of detachable bonds, and release with time or can be removed by active mechanical or chemical processes. In other formulations, the active compound, or a pharmaceutically acceptable salt thereof, may be in a permanently immobilized form that presents the active compound at the implantation site.

In certain embodiments, the active compound, or a pharmaceutically acceptable salt thereof, may be incorporated with polymer compositions during the formation of biocompatible coatings for medical devices, such as stents. The coatings produced from these components are typically homogeneous and are useful for coating a number of devices designed for implantation.

The polymer may be either a biostable or a bioabsorbable polymer depending on the desired rate of release or the desired degree of polymer stability, but frequently a bioabsorbable polymer is suitable for this embodiment because, unlike a biostable polymer, it will typically not be present long after implantation to cause any adverse, chronic local response. Bioabsorbable polymers that could be used include, but are not limited to, poly(L-lactic acid), polycaprolactone, polyglycolide (PGA), poly(lactide-co-glycolide) (PLLA/PGA), poly(hydroxybutyrate), poly(hydroxybutyrate-co-valerate), polydioxanone, polyorthoester, polyanhydride, poly(glycolic acid), poly(D-lactic acid), poly(L-lactic acid), poly(D, L-lactic acid), poly(D, L-lactide) (PLA), poly(L-lactide) (PLLA), poly(glycolic acid-co-trimethylene carbonate) (PGA/PTMC), polyethylene oxide (PEO), polydioxanone (PDS), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen and hyaluronic acid, polyepsilon caprolactone, polyhydroxy butyric acid, polyorthoesters, polyacetals, polydihydropyrans, polycyanoacrylates, cross linked or amphipathic block copolymers of hydrogels, and other suitable bioabsorbable poplymers known in the art. Also, biostable polymers with a relatively low chronic tissue response such as polyurethanes, silicones, and polyesters could be used, and other polymers could also be used if they can be dissolved and cured or polymerized on the medical device such as polyolefins, polyisobutylene and ethylene-alphaolefin copolymers; acrylic polymers and copolymers, vinyl halide polymers and copolymers, such as polyvinyl chloride; polyvinylpyrrolidone; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; pyran copolymer; polyhydroxy-propyl-methacrylamide-phenol; polyhydroxyethyl-aspartamide-phenol; polyethyleneoxide-polylysine substituted with palmitoyl residues; polyamides, such as Nylon 66 and polycaprolactam; alkyd resins, polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

Polymers and semipermeable polymer matrices may be formed into shaped articles, such as valves, stents, tubing, prostheses and the like.

In certain embodiments of the invention, the compound of the disclosure, or pharmaceutically acceptable salt thereof, is coupled to a polymer or semipermeable polymer matrix that is formed as a stent or stent-graft device.

Typically, polymers are applied to the surface of an implantable device by spin coating, dipping, or spraying. Additional methods known in the art can also be utilized for this purpose. Methods of spraying include traditional methods as well as microdeposition techniques with an inkjet type of dispenser. Additionally, a polymer can be deposited on an implantable device using photo-patterning to place the polymer on only specific portions of the device. This coating of the device provides a uniform layer around the device which allows for improved diffusion of various analytes through the device coating.

In certain embodiments of the invention, the compound of the disclosure, or pharmaceutically acceptable salt thereof, is formulated for release from the polymer coating into the environment in which the medical device is placed. Preferably, the active compound, or a pharmaceutically acceptable salt thereof, is released in a controlled manner over an extended time frame (e.g., months) using at least one of several well-known techniques involving polymer carriers or layers to control elution. Some of these techniques are described in U.S. Patent Application 2004/0243225A1, the entire disclosure of which is incorporated herein in its entirety.

Moreover, as described for example in U.S. Pat. No. 6,770,729, which is incorporated herein in its entirety, the reagents and reaction conditions of the polymer compositions can be manipulated so that the release of the active compound, or a pharmaceutically acceptable salt thereof, from the polymer coating can be controlled. For example, the diffusion coefficient of the one or more polymer coatings can be modulated to control the release of the active compound, or a pharmaceutically acceptable salt thereof, from the polymer coating. In a variation on this theme, the diffusion coefficient of the one or more polymer coatings can be controlled to modulate the ability of an analyte that is present in the environment in which the medical device is placed (e.g., an analyte that facilitates the breakdown or hydrolysis of some portion of the polymer) to access one or more components within the polymer composition (and for example, thereby modulate the release of the active compound, or a pharmaceutically acceptable salt thereof, from the polymer coating). Yet another embodiment of the invention includes a device having a plurality of polymer coatings, each having a plurality of diffusion coefficients. In such embodiments of the invention, the release of the active compound, or a pharmaceutically acceptable salt thereof, from the polymer coating can be modulated by the plurality of polymer coatings.

In yet another embodiment of the invention, the release of the active compound, or a pharmaceutically acceptable salt thereof, from the polymer coating is controlled by modulating one or more of the properties of the polymer composition, such as the presence of one or more endogenous or exogenous compounds, or alternatively, the pH of the polymer composition. For example, certain polymer compositions can be designed to release an active compound, or a pharmaceutically acceptable salt thereof, in response to a decrease in the pH of the polymer composition.

### Kits

Kits comprising a compound of the disclosure are also provided. In certain embodiments, a kit is provided comprising a compound of the disclosure, or a pharmaceutically acceptable salt thereof, and at least one of packaging material, and instructions for administering the compound of the disclosure or the pharmaceutically acceptable salt thereof and the other therapeutic agent or agents to a mammal to treat or prevent a disease or condition that would benefit from GLP-1R activation. In certain embodiments, the mammal is a human. In a specific embodiment, the mammal is a human.

In another embodiment, a kit is provided comprising a compound of the disclosure, or a pharmaceutically acceptable salt thereof, and at least one of at least one other therapeutic agent, packaging material, and instructions for administering the compound of the disclosure or the pharmaceutically acceptable salt thereof and the other therapeutic agent or agents to a mammal to treat or prevent a disease or condition that would benefit from GLP-1R activation. In a specific embodiment, the mammal is a human.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof.

### EXAMPLES

The invention now being generally described, it will be more readily understood by reference to the following examples which are included merely for purposes of illustration of certain aspects and embodiments of the present invention, and are not intended to limit the invention.

### Preparation 1—Synthetic Scheme

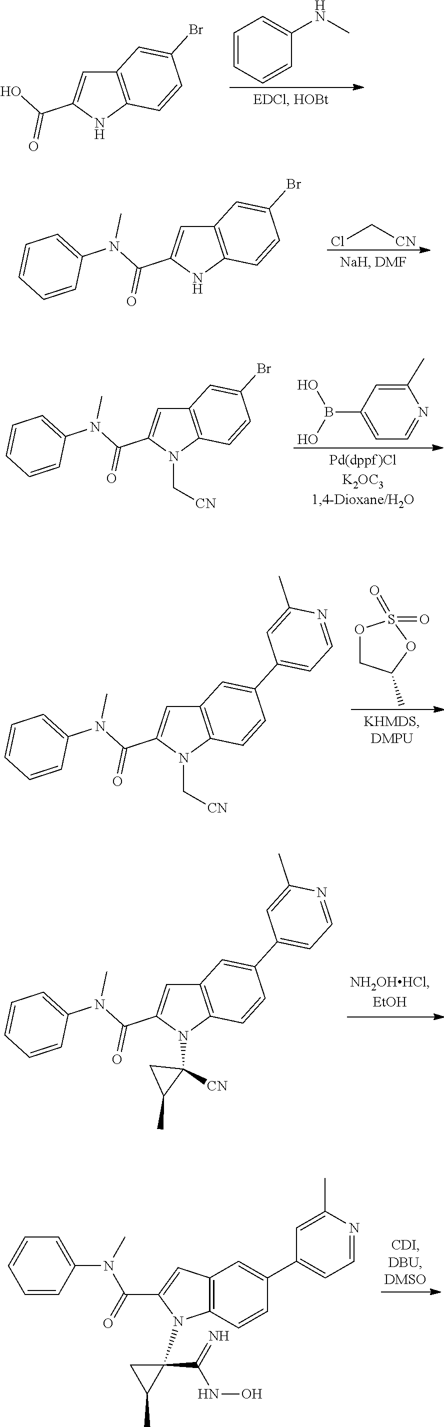

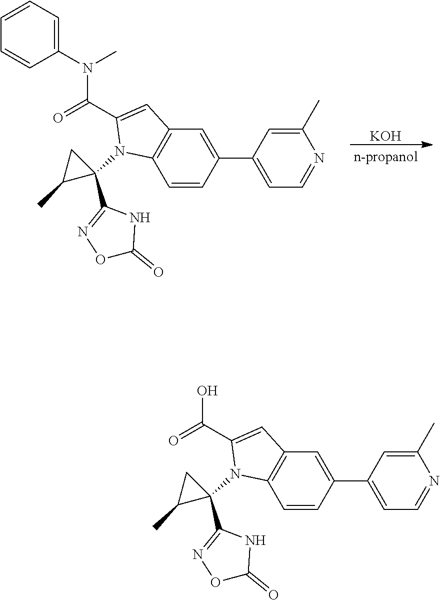

### Preparation 1—Synthetic Procedure

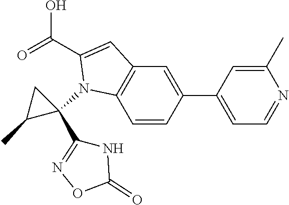

Step A: To a solution of 5-bromo-2-indolecarboxylic acid (20 g, 83.3 mmol) and N-methylaniline (9.82 g, 91.6 mmol) in DMF (0.2 L) at 0° C. were added EDCI (31.9 g, 167 mmol) and HOBT (22.5 g, 167 mmol). The mixture was warmed to ambient temperature and stirred for 16 hours. The mixture was then diluted with water (200 mL) and the precipitate was collected by vacuum filtration. The solid was washed with water (100 mL) then dried to give N-methyl-N-phenyl-5-bromo-2-indolecarboxamide (24 g, 65.6 mmol) as an off-white solid. m/z (ESI-pos) M+1=329.0, 331.0.

Step B: To a solution of N-methyl-N-phenyl-5-bromo-2-indolecarboxamide (10 g, 27.3 mmol) in DMF (50 mL) at 0° C. was added sodium hydride (60% w/w, 1.97 g, 49.2 mmol). The mixture was stirred at 0° C. for 30 mins and then chloroacetonitrile (4.13 g, 54.7 mmol) was added dropwise at this temperature. The resulting mixture was allowed to warm to ambient temperature and stirred for 16 hours. The reaction was quenched with water, extracted with EtOAc (2×). The combined EtOAc layers were washed with brine (2×), dried over Na₂SO₄, filtered and concentrated under reduced pressure. The crude product was purified by column chromatography (0-25% EtOAc in PE) to give N-methyl-N-phenyl-5-bromo-1-(cyanomethyl)-2-indolecarboxamide (9 g, 22 mmol) as a yellow solid. m/z (ESI-pos) M+1=368.0, 370.0

Step C: To a mixture of N-methyl-N-phenyl-5-bromo-1-(cyanomethyl)-2-indolecarboxamide (9 g, 24.4 mmol), (2-methyl-4-pyridyl)boranediol (4.02 g, 29.3 mmol) and K₂CO₃ (6.76 g, 48.9 mmol) in 1,4-dioxane (180 mL) and water (36 mL) was added Pd(dppf)Cl₂ dichloromethane complex (2 g, 2.44 mmol). The mixture was stirred at 80° C. for 2.5 h and then the reaction mixture was cooled to ambient temperature. The mixture was diluted with ethyl acetate and washed with water (2×). The aqueous layer was back extracted with ethyl acetate (2×). The combined organic layers were washed with brine solution, dried over anhydrous Na₂SO₄, filtered and concentrated. The crude residue was purified by column chromatography (0-50% EtOAc in PE) to give N-methyl-N-phenyl-1-(cyanomethyl)-5-(2-methyl-4-pyridyl)-2-indolecarboxamide (5 g, 13.1 mmol) as a yellow solid. m/z (ESI-pos) M+1=381.2

Step D: To a solution of N-methyl-N-phenyl-1-(cyanomethyl)-5-(2-methyl-4-pyridyl)-2-indolecarboxamide (5 g, 13.1 mmol) in DMPU (250 mL) at was added (R)-4-methyl-1,3,2-dioxathiolane 2,2-dioxide (4.54 g, 32.9 mmol), followed by dropwise addition of KHMDS (1.0 M in THF, 52.6 mL, 52.6 mmol). The mixture was stirred at 0° C. for 2 hours and quenched with aqueous NH₄Cl. The mixture was extracted with EtOAc (2×). The combined EtOAc layers were dried over Na₂SO₄, filtered and concentrated. The crude product was purified by column chromatography (0-25% EtOAc in PE) to give N-methyl-N-phenyl-1-[(2S)-1-cyano-2-methylcyclopropyl]-5-(2-methyl-4-pyridyl)-2-indolecarboxamide (4.5 g, 10.7 mmol) as a yellow liquid. m/z (ESI-pos) M+1=421.3

Step E: To a solution of N-methyl-N-phenyl-1-[(2S)-1-cyano-2-methylcyclopropyl]-5-(2-methyl-4-pyridyl)-2-indolecarboxamide (4 g, 9.51 mmol) in EtOH (77.7 mL) were added Hydroxylamine hydrochloride (3.31 g, 47.6 mmol) and K₂CO₃ (7.23 g, 52.3 mmol). The mixture was stirred at 100° C. for 2 hours and then concentrated under reduced pressure. The residue was diluted with H₂O and the precipitate was collected by vacuum filtration to give N-methyl-N-phenyl-1-[(2S)-1-(hydroxyamino) carboximidoyl-2-methylcyclopropyl]-5-(2-methyl-4-pyridyl)-2-indolecarboxamide (3 g, 6.61 mmol) as a white solid. m/z (ESI-pos) M+1=454.2

Step F: To a solution of N-methyl-N-phenyl-1-[(2S)-1-(hydroxyamino) carboximidoyl-2-methylcyclopropyl]-5-(2-methyl-4-pyridyl)-2-indolecarboxamide (3 g, 6.61 mmol) in DMSO (61.8 mL) was added CDI (2.15 g, 13.2 mmol) and DBU (2.52 g, 16.5 mmol). The mixture was stirred at 80° C. for 2 hours and then cooled to ambient temperature, diluted with ethyl acetate, and washed with water (2×). The aqueous layer was extracted with ethyl acetate (2×). The combined organic layers were washed with brine solution, dried over anhydrous Na₂SO₄, filtered and concentrated to give the crude compound, which was purified by column chromatography (0-80% EA in PE) to give N-methyl-N-phenyl-1-[(2S)-2-methyl-1-(5-oxo-4,5-dihydro-1,2,4-oxadiazol-3-yl)cyclopropyl]-5-(2-methyl-4-pyridyl)-2-indolecarboxamide (2.1 g, 4.38 mmol) as a yellow solid. m/z (ESI-pos) M+1=480.3

Step G: To a solution of N-methyl-N-phenyl-1-[(2S)-2-methyl-1-(5-oxo-4,5-dihydro-1,2,4-oxadiazol-3-yl)cyclopropyl]-5-(2-methyl-4-pyridyl)-2-indolecarboxamide (2.1 g, 4.38 mmol) in n-propanol (63 mL) was added KOH (2.46 g, 43.8 mmol). The mixture was stirred at 130° C. for 16 h, then additional KOH (1.23 g, 21.9 mmol) was added and stirred at 130° C. for another 16 h. The reaction mixture was concentrated under reduced pressure. The residue was diluted with H₂O (5 mL) at 0°, then 6N aq. HCl was added dropwise to the mixture until solid began to precipitate, then additional 1N aq. HCl was added dropwise until the pH was ~5-6. The precipitate was collected by vacuum filtration, to give 1-[(2S)-2-methyl-1-(5-oxo-4,5-dihydro-1,2,4-oxadiazol-3-yl)cyclopropyl]-5-(2-methyl-4-pyridyl)-2-indolecarboxylic acid (1.04 g, 2.66 mmol) as a white solid. m/z (ESI-pos) M+1=381.2, ¹H NMR (400 MHz, DMSO) δ 8.45 (d, J=5.2 Hz, 1H), 8.00 (s, 1H), 7.66 (dd, J=1.6, 8.8 Hz, 1H), 7.58 (s, 1H), 7.52 (d, J=8.4 Hz, 1H), 7.49 (d, J=5.2 Hz, 1H) 6.81 (s, 1H), 2.52 (s, 3H), 1.71-1.63 (m, 2H), 1.50-1.47 (m, 1H) 1.11 (d, J=6.0 Hz, 3H).

### Preparation 2—Synthetic Scheme

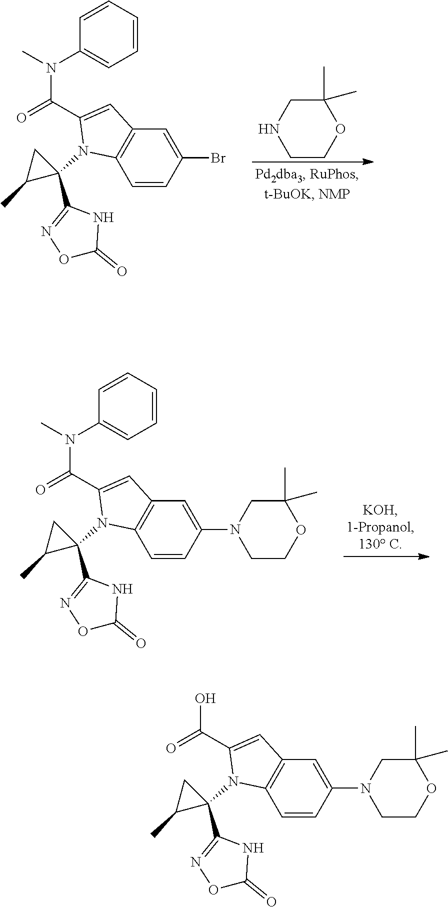

### Preparation 2—Synthetic Procedure

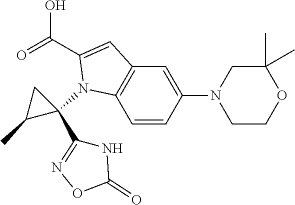

Step A: To a mixture of 5-bromo-N-methyl-1-(1-(5-oxo-4,5-dihydro-1,2,4-oxadiazol-3-yl)cyclopropyl)-N-phenyl-1H-indole-2-carboxamide (500 mg, 1.11 mmol), 2,2-dimethylmorpholine (165 mg, 1.14 mmol) in N-Methyl-2-pyrrolidone (25 mL) were added t-BuONa (319 mg, 3.32 mmol), 2-Dicyclohexylphosphino-2′,6′-di-i-propoxy-1,1′-biphenyl (103 mg, 0.221 mmol) and tris(dibenzylideneacetone) dipalladium (101 mg, 0.11 mmol) under N₂ protection and then stirred at 100° C. for 0.5 h. The reaction mixture was cooled to ambient temperature, quenched with water and extracted with EtOAc (3×). The combined organic layers were washed with brine, dried over Na₂SO₄, filtered and the filtrate was concentrated to give a residue, which was purified by column chromatography (0-50% EtOAc in PE) to give 5-(2,2-dimethylmorpholino)-N-methyl-1-((1S,2S)-2-methyl-1-(5-oxo-4,5-dihydro-1,2,4-oxadiazol-3-yl)cyclopropyl)-N-phenyl-1H-indole-2-carboxamide (500 mg, 0.84 mmol) as a yellow oil. m/z (ESI-pos) M+1=502.3

Step B: To a solution of 5-(2,2-dimethylmorpholino)-N-methyl-1-((1S,2S)-2-methyl-1-(5-oxo-4,5-dihydro-1,2,4-oxadiazol-3-yl)cyclopropyl)-N-phenyl-1H-indole-2-carboxamide (220 mg, 439 μmol) in 1-propanol (5 mL) was added potassium hydroxide (426 mg, 4.39 mmol). The reaction mixture was stirred at 130° C. for 36 h. The reaction mixture was cooled to ambient temperature and concentrated to remove 1-propanol. The residue was suspended in water (30 mL) and extracted with EtOAc (10 mL, the organics were discarded). The aqueous layer was acidified to pH about 5 with 1N aq. HCl and extracted with EtOAc (3×). The combined organic layers were washed with brine, dried over Na₂SO₄, filtered and the filtrate was concentrated to give crude 5-(2,2-dimethylmorpholino)-1-((1S,2S)-2-methyl-1-(5-oxo-4,5-dihydro-1,2,4-oxadiazol-3-yl)cyclopropyl)-1H-indole-2-carboxylic acid (70 mg, 153 μmol) as a yellow solid. m/z (ESI-pos) M+1=411.2

### Preparation 3-Synthetic Scheme

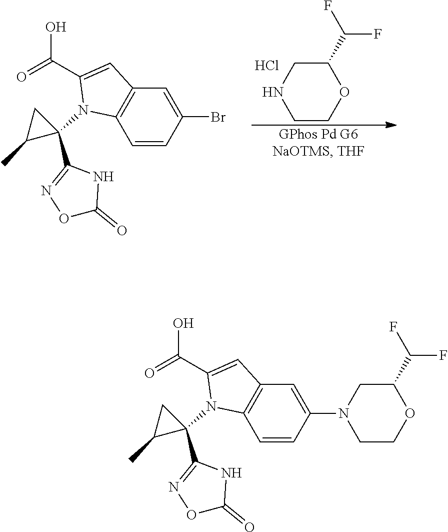

### Preparation 3-Synthetic Procedure

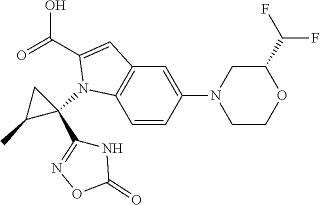 [SMILES: C[C@H]1C[C@]1(c1noc(=O)[nH]1)n1c(C(=O)O)cc2cc(N3CCO[C@@H](C(F)F)C3)ccc21]

Step A: To a mixture of 5-bromo-1-[(1S,2S)-2-methyl-1-(5-oxo-4,5-dihydro-1,2,4-oxadiazol-3-yl)cyclopropyl]-2-indolecarboxylic acid (315 mg, 833 μmol), (R)-2-(difluoromethyl)morpholine hydrochloride (214 mg, 1.25 mmol), bromidopalladium(1+) 2-(trimethylsilyl)ethyl 1-benzenid-4-carboxylate-(3-tert-butoxy-2′,6′-diisopropyl-6-methoxy-2-biphenylyl)dicyclohexylphosphine (1/1) (39.3 mg, 41.6 μmol), sodium trimethylsilanolate (467 mg, 4.16 mmol), was added tetrahydrofuran (2.78 mL). The mixture was sparged for 5 minutes with Ar and then heated to 70° C. for 16 hours. The reaction was cooled and diluted with EtOAc. The organics were washed twice with 1M aq HCl, then brine. Organics were dried over magnesium sulfate, filtered, and concentrated in vacuo. The crude residue was purified by reverse-phase prep-HPLC purification eluting with 20-80% MeCN+0.1% FA in water+0.1% FA. Desired fractions were concentrated to afford 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid (302 mg, 695 μmol). m/z (ESI-pos) M+1=435.3; ¹H NMR (400 MHz, CHLOROFORM-D) δ 11.69 (s, 1H), 10.61 (s, 1H), 7.63 (d, J=9.1 Hz, 1H), 7.24-7.16 (m, 2H), 7.11 (d, J=2.3 Hz, 1H), 5.82 (td, J=55.3, 4.3 Hz, 1H), 4.10 (d, J=11.3 Hz, 1H), 4.02-3.83 (m, 2H), 3.49 (d, J=11.5 Hz, 1H), 3.34 (d, J=11.9 Hz, 1H), 2.90 (td, J=11.6, 3.3 Hz, 1H), 2.85-2.75 (m, 1H), 1.90-1.74 (m, 2H), 1.54-1.46 (m, 1H), 1.26 (d, J=5.9 Hz, 3H); 19F NMR (376 MHz, CHLOROFORM-D) δ −127.79 (ddd, J=295.9, 55.1, 9.2 Hz), −131.12 (ddd, J=295.9, 55.4, 11.0 Hz).

### Preparation 4—Synthetic Scheme

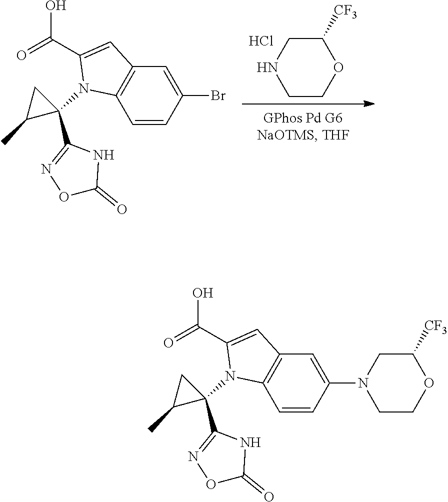

### Preparation 4—Synthetic Procedure

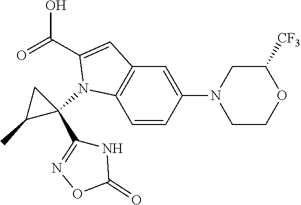 [SMILES: C[C@H]1C[C@]1(c1noc(=O)[nH]1)n1c(C(=O)O)cc2cc(N3CCO[C@@H](C(F)(F)F)C3)ccc21]

Step A: To a mixture of 5-bromo-1-[(1S,2S)-2-methyl-1-(5-oxo-4,5-dihydro-1,2,4-oxadiazol-3-yl)cyclopropyl]-2-indolecarboxylic acid (2 g, 5.29 mmol), (R)-2-(trifluoromethyl)morpholine hydrochloride (1.3 g, 6.87 mmol), bromidopalladium(1+) 2-(trimethylsilyl)ethyl 1-benzenid-4-carboxylate-(3-tert-butoxy-2′,6′-diisopropyl-6-methoxy-2-biphenylyl)dicyclohexylphosphine (1/1) (250 mg, 264 μmol), and sodium trimethylsilanolate (2.97 g, 26.4 mmol), was added tetrahydrofuran (17.6 mL). The mixture was sparged for 5 minutes then heated to 70° C. for 16 hours. The reaction was cooled and diluted with EtOAc. The organics were washed twice with 1M aq HCl, then brine. Organics were dried over magnesium sulfate, filtered, and concentrated in vacuo. The crude residue was purified by reverse-phase prep-HPLC purification eluting with 20-75% MeCN+0.1% FA in water+0.1% FA. Desired fractions were concentrated to afford 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid (1.58 g, 3.49 mmol). m/z (ESI-pos) M+1=453.3; ¹H NMR (400 MHz, CHLOROFORM-D) δ 11.91 (s, 1H), 10.66 (s, 1H), 7.64 (d, J=9.0 Hz, 1H), 7.24-7.16 (m, 2H), 7.12 (d, J=2.3 Hz, 1H), 4.20-4.08 (m, 2H), 3.90 (td, J=11.4, 2.6 Hz, 1H), 3.58-3.50 (m, 1H), 3.39-3.31 (m, 1H), 2.98-2.81 (m, 2H), 1.90-1.74 (m, 2H), 1.58-1.46 (m, 1H), 1.26 (d, J=6.0 Hz, 3H).

### Preparation 5—Synthetic Scheme

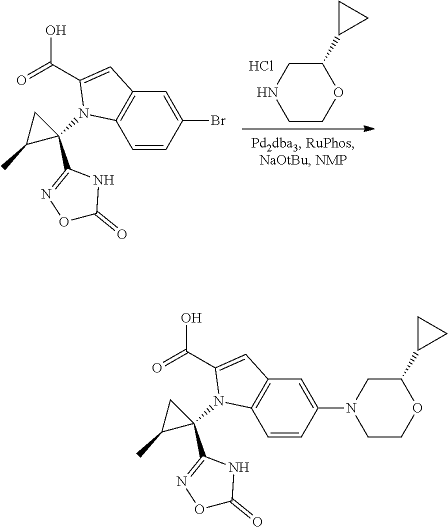

### Preparation 5—Synthetic Procedure

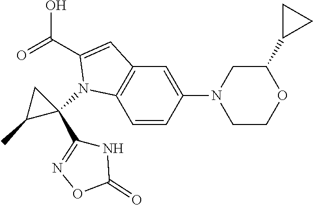 [SMILES: C[C@H]1C[C@]1(c1noc(=O)[nH]1)n1c(C(=O)O)cc2cc(N3CCO[C@@H](C4CC4)C3)ccc21]

Step A: A 50 mL round bottom flask was charged with(S)-4-chloro-2-cyclopropyl-4λ⁵-morpholine (833 mg, 5.16 mmol), Pd₂dba₃ (182 mg, 198 μmol), dicyclohexyl(2′,6′-diisopropoxy-2-biphenylyl)phosphine (185 mg, 397 μmol), NaOtBu (1.91 g, 19.8 mmol) and N-methyl-2-pyrrolidone (13.2 mL). The mixture was sparged for 5 minutes with Ar, then 5-bromo-1-[(1S,2S)-2-methyl-1-(5-oxo-4,5-dihydro-1,2,4-oxadiazol-3-yl)cyclopropyl]-2-indolecarboxylic acid (1.5 g, 3.97 mmol) was added and the mixture was heated to 100° C. where it stirred for 1 hour. The mixture was cooled to ambient temperature and diluted with water and extracted with EtOAc (3×). The EtOAc layer was discarded. The aqueous phase was then treated with 1M HCl (14 mL) until pH=~1, and the mixture was extracted with EtOAc (4×). The combined organics were dried over Na₂SO₄, filtered through Celite, and concentrated. The product was purified by Prep HPLC (Isco ACCQ Prep, Gemini column NX-C18, 250×50 mm, 120 ml/min, 35-minute separation, 10 to 60% H₂O/ACN with 0.1% Formic acid buffer), to give 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid (1.39 g, 3.28 mmol). m/z (ESI-pos) M+1=425.3, ¹H NMR (400 MHz, CHLOROFORM-D) δ 10.64 (s, 1H), 7.60 (d, J=9.1 Hz, 1H), 7.22-7.01 (m, 3H), 4.07-4.00 (m, 1H), 3.77 (td, J=11.4, 2.5 Hz, 1H), 3.50 (d, J=11.5 Hz, 1H), 3.32 (d, J=11.7 Hz, 1H), 2.98-2.89 (m, 1H), 2.83 (td, J=11.6, 3.2 Hz, 1H), 2.69 (dd, J=11.7, 10.1 Hz, 1H), 1.88-1.73 (m, 2H), 1.57-1.45 (m, 1H), 1.28-1.22 (m, 4H), 0.95 (dtd, J=13.4, 8.6, 5.0 Hz, 1H), 0.66-0.50 (m, 2H), 0.45 (dq, J=9.1, 4.7 Hz, 1H), 0.29 (dq, J=9.6, 4.8 Hz, 1H).

### Preparation 6—Synthetic Scheme

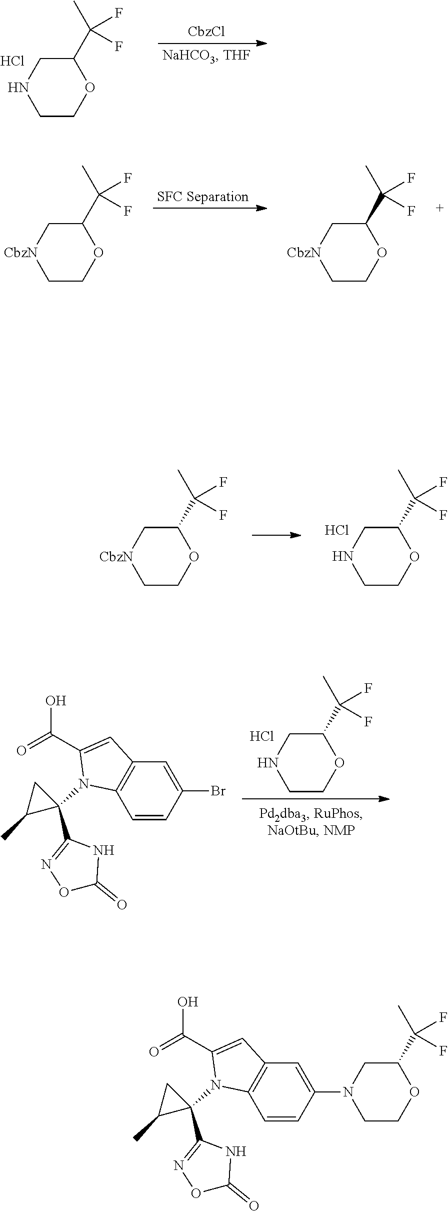

### Preparation 6—Synthetic Procedure

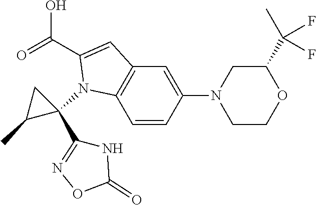 [SMILES: C[C@H]1C[C@]1(c1noc(=O)[nH]1)n1c(C(=O)O)cc2cc(N3CCO[C@@H](C(C)(F)F)C3)ccc21]

Step A: A solution of 2-(1,1-difluoroethyl)morpholine hydrogen chloride (5.0 g, 26.7 mmol) in tetrahydrofuran (53.3 mL) and water (17.8 mL) was treated with sodium hydrogencarbonate (8.96 g, 107 mmol) followed by phenylmethyl chloroformate (4.13 mL, 29.3 mmol). The mixture was then stirred at ambient temperature overnight. The mixture was then diluted with water and extracted with EtOAc (3×). The combined organic layers were then washed with brine (2×), dried over Na₂SO₄, filtered and concentrated. The crude product was then purified via column chromatography (0-15% EtOAc/heptane) to afford benzyl 2-(1,1-difluoroethyl)-4-morpholinecarboxylate (6.72 g, 23.6 mmol) as a thick, colorless oil. m/z (ESI-pos) M+1=286.2, ¹H NMR (400 MHz, CHLOROFORM-D) δ 7.39-7.29 (m, 5H), 5.19-5.12 (m, 2H), 4.31-3.82 (m, 4H), 3.65-3.43 (m, 2H), 3.00 (br s, 1H), 1.62 (t, J=19.3 Hz, 3H).

Step B: Benzyl 2-(1,1-difluoroethyl)-4-morpholinecarboxylate (5.95 g) was then separated by SFC using a Berger MG-II Preparative SFC with a Regis® Whelk-O1 (R,R) (250×20 mm) column @ 35° C. The mobile phase CO₂ modifier was 5% [1:1] methanol:ethanol (neat) w/8 mL/min post-BPR make-up flow ethanol with a flow rate of 60 mL/min.

Peak 1: benzyl(S)-2-(1,1-difluoroethyl)-4-morpholinecarboxylate (2.40 g, stereochemistry arbitrarily assigned), [α]_{D}²³+7.9° (c 1.8, MeOH).

Peak 2: benzyl (R)-2-(1,1-difluoroethyl)-4-morpholinecarboxylate (2.50 g, stereochemistry arbitrarily assigned), [α]_{D}²³−7.9° (c 2.7, MeOH).

Step C: A solution of benzyl (R)-2-(1,1-difluoroethyl)-4-morpholinecarboxylate (SFC separation Peak 2, 2.45 g, 8.59 mmol) in ethyl acetate (28.6 mL) was treated with Pd/C (5% w/w, 274 mg, 129 μmol). The mixture was then purged of air by placing the system under vacuum and then introducing hydrogen gas (3×). The vial was sealed and the mixture was stirred under a H₂ atmosphere (balloon pressure) for 4 hours. The hydrogen atmosphere was removed and the mixture was briefly purged with N₂. The mixture was then filtered through celite and the filtrate was treated with hydrogen chloride (2.15 mL, 4.0M in dioxane, 8.59 mmol) and heptane (25 mL). The mixture was concentrated in vacuo. The crude product was then dissolved in CH₂Cl₂ and MTBE and the mixture was concentrated in vacuo to a thick oil that was placed under vacuum overnight to form a tan solid. The solid was then sonicated in MTBE and the solid was isolated by vacuum filtration. The solid was then washed with heptane and dried in vacuo to provide (R)-2-(1,1-difluoroethyl)morpholine hydrogen chloride (1.52 g, 8.1 mmol) as an off-white solid. m/z (ESI-pos) M+1=152.1, ¹H NMR (400 MHz, CHLOROFORM-D) δ 10.23 (s, 2H), 4.27-3.95 (m, 3H), 3.63-3.45 (m, 1H), 3.36 (d, J=12.9 Hz, 1H), 3.18-2.94 (m, 2H), 1.65 (t, J=19.2 Hz, 3H), [α]_{D}²³+7.7° (c 1.15, MeOH).

Step D: A mixture of (R)-2-(1,1-difluoroethyl)morpholine hydrogen chloride (957 mg, 5.16 mmol), Pd₂dba₃ (182 mg, 198 μmol), dicyclohexyl(2′,6′-diisopropoxy-2-biphenylyl)phosphine (185 mg, 397 μmol), NaOtBu (1.91 g, 19.8 mmol) and N-methyl-2-pyrrolidone (13.2 mL) was sparged for 5 minutes with Ar. Then 5-bromo-1-[(1S,2S)-2-methyl-1-(5-oxo-4,5-dihydro-1,2,4-oxadiazol-3-yl)cyclopropyl]-2-indolecarboxylic acid (1.5 g, 3.97 mmol) was added and the mixture was heated to 100° C. under an Ar atmosphere where it stirred for 2 hours. The mixture was cooled to ambient temperature and diluted with water and EtOAc and the mixture was filtered through celite. The layers were separated and the aqueous phase was extracted with EtOAc (2×). The EtOAc layers were discarded. The aqueous phase was then treated with 1M HCl (15 mL) until pH=~1, and the mixture was extracted with EtOAc (4×). The combined organics were dried over Na₂SO₄, filtered through Celite, and the filtrate was concentrated. The product purified by Prep HPLC (Isco ACCQ Prep, Gemini column NX—C18, 250×50 mm, 120 ml/min, 35-minute separation, 10 to 60% H2O/ACN with 0.1% Formic acid buffer) to give 5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolecarboxylic acid (1.46 g, 3.26 mmol). m/z (ESI-pos) M+1=449.3, ¹H NMR (400 MHz, CHLOROFORM-D) δ 10.66 (s, 1H), 7.61 (d, J=9.1 Hz, 1H), 7.21-7.08 (m, 3H), 4.15-4.06 (m, 1H), 3.91-3.77 (m, 2H), 3.56 (d, J=11.6 Hz, 1H), 3.34 (d, J=11.9 Hz, 1H), 2.86 (td, J=11.8, 3.3 Hz, 1H), 2.75 (t, J=11.1 Hz, 1H), 1.89-1.76 (m, 2H), 1.68 (t, J=19.3 Hz, 3H), 1.56-1.43 (m, 1H), 1.26-1.23 (m, 3H).

### Preparation 7—Synthetic Scheme

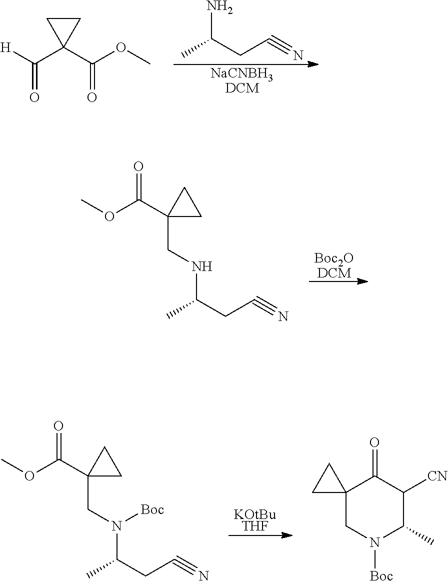

### Preparation 7—Synthetic Procedure

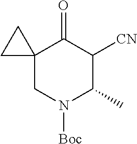 [SMILES: C[C@H]1C(C#N)C(=O)C2(CC2)CN1C(=O)OC(C)(C)C]

Step A: A solution of(S)-3-aminobutyronitrile-hydrogen chloride (1/1) (17 g, 141 mmol) and methyl 1-formylcyclopropanecarboxylate (19.9 g, 155 mmol) in DCM (300 mL) was stirred for 30 minutes, then NaCNBH₃ (11 g, 183 mmol) was added and stirred overnight at room temperature. The solution was then filtered through celite and partitioned between sat. aq. NH₄Cl and DCM. The organics were dried with sodium sulfate, filtered, concentrated, and taken forward as the crude material. m/z (ESI-pos) [M+1]=197.2

Step B: A solution of crude methyl 1-{[(S)-2-cyano-1-methylethylamino]methyl}cyclopropanecarboxylate (20 g, 102 mmol) and Boc₂O (28.9 g, 132 mmol) in DCM (300 mL) was stirred overnight at room temperature. The solution was then concentrated and purified by silica gel chromatography (5-95% EtOAc/heptanes using ELSD to monitor peaks), and the product was concentrated to give methyl 1-({tert-butyl[(S)-2-cyano-1-methylethyl](oxycarbonylamino)}methyl)cyclopropanecarboxylate (9 g, 30.4 mmol). m/z (ESI-pos) [M+1-CH₃]=283.2

Step C: To a solution of methyl 1-({tert-butyl[(S)-2-cyano-1-methylethyl](oxycarbonylamino)}methyl)cyclopropanecarboxylate (8.3 g, 28 mmol) in THF (120 mL) at 0° C. was added KOtBu (3.46 g, 30.8 mmol) and stirred for 2 hours at room temperature. The solution was then quenched with aq. sat. NH₄Cl and partitioned between DCM and water. The organics were dried with sodium sulfate, filtered, and concentrated to give tert-butyl (6S)-7-cyano-6-methyl-8-oxo-5-aza-5-spiro[2.5]octanecarboxylate (7.4 g, 28 mmol). m/z (ESI-pos) [M+1-tBu]=209.1

### Preparation 8—Synthetic Scheme

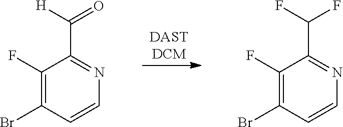 [SMILES: O=Cc1nccc(Br)c1F ; Fc1c(Br)ccnc1C(F)F]

### Preparation 8—Synthetic Procedure

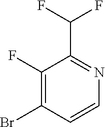 [SMILES: Fc1c(Br)ccnc1C(F)F]

Step A: To a solution of 4-bromo-3-fluoro-2-pyridinecarbaldehyde (153 mg, 750 μmol) in 4 mL of DCM in a vial was slowly added DAST (495 μL, 3.75 mmol) and stirred for 30 minutes at room temperature. The solution was then carefully quenched with sat. NaHCO₃ and partitioned between water and DCM. The organics were concentrated and purified by silica gel chromatography (0-20% EtOAc/heptanes), and the product was concentrated to give 4-bromo-2-(difluoromethyl)-3-fluoropyridine (50 mg, 221 μmol). ¹H NMR (400 MHz, chloroform-D) δ 8.28 (d, J=5.1 Hz, 1H), 7.67 (tt, J=5.0, 0.9 Hz, 1H), 6.77 (t, J=53.5 Hz, 1H).

### Preparation 9—Synthetic Scheme

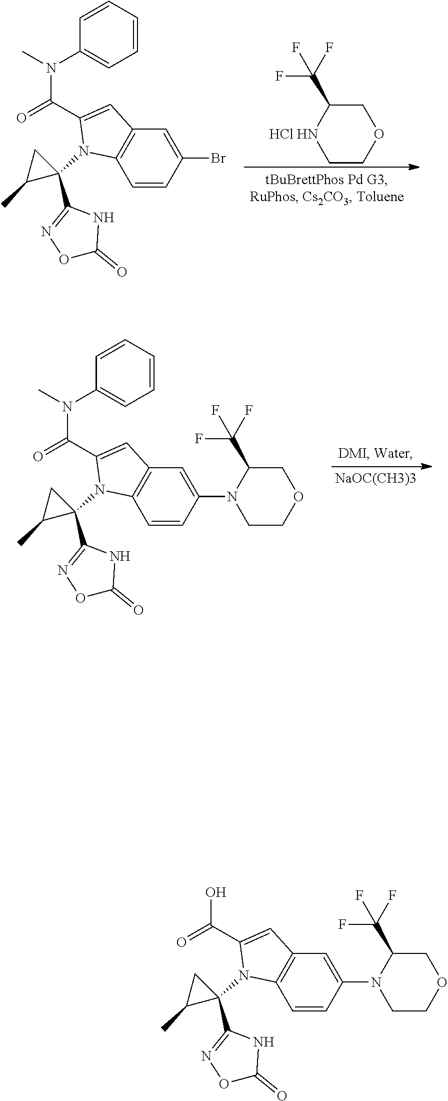

### Preparation 9—Synthetic Procedure

Step A: To a mixture of N-methyl-N-phenyl-5-bromo-1-[(2S)-1-cyano-2-methylcyclopropyl]-2-indolecarboxamide (290 mg, 710 μmol), (R)-3-(trifluoromethyl)morpholine hydrogen chloride (272 mg, 1.42 mmol) in Toluene (4 mL) were added dicaesium carbonate (1.16 g, 3.55 mmol), RuPhos (66.3 mg, 142 μmol) and tBuBrettPhos Pd G3 (60.8 mg, 71 μmol) under N₂ protection and then stirred at 90° C. for 16 h. The reaction mixture was cooled to ambient temperature, filtered over celite. The filtrate was concentrated to give a residue, which was purified by column chromatography (0-50% EtOAc in PE) to give N-methyl-N-phenyl-1-[(2S)-1-cyano-2-methylcyclopropyl]-5-[(R)-3-(trifluoromethyl)-4-morpholinyl]-2-indolecarboxamide (98 mg, 203 μmol) as a yellow foam. m/z (ESI-pos) M+1=483.3

Step B: To a solution of N-methyl-N-phenyl-1-[(2S)-1-cyano-2-methylcyclopropyl]-5-[(R)-3-(trifluoromethyl)-4-morpholinyl]-2-indolecarboxamide (50 mg, 92.3 μmol) in 1,3-Dimethyl-2-imidazolidinone (1.79 mL, 51.7 mmol) and water (2.08 μL, 115 μmol) was added Sodium tert-butoxide (26.6 mg, 277 μmol). The reaction mixture was stirred at 85° C. for 24 h. The pH value of the mixture was adjusted to around 3 by adding 6M HCl. The mixture was extracted with 3:1 IPA/chloroform in Water. collected organic layer and dried over sodium sulfate. concentrated, the crude residue was directly loaded (with some MeCN, MeOH, and H2O) onto the reverse phase column and purified by Prep HPLC (Isco ACCQ Prep, Gemini column NX—C18, 250×21 mm, 20 ml/min, 45 minute separation, 20 to 80% H2O/ACN with 0.1% Formic acid buffer), fractions were collected and lyophilized to give. 1-[(2S)-2-methyl-1-(5-oxo-4,5-dihydro-1,2,4-oxadiazol-3-yl)cyclopropyl]-5-[(R)-3-(trifluoromethyl)-4-morpholinyl]-2-indolecarboxylic acid (10 mg, 22.1 μmol) m/z (ESI-pos) M+1=453.2.

### Preparation 10—Synthetic Scheme

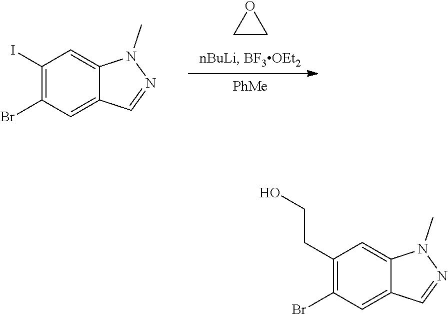

### Preparation 10—Synthetic Procedure

Step A: To a 40 mL vial was added 5-bromo-6-iodo-1-methyl-1H-indazole (633 mg, 1.88 mmol) and toluene (18.8 mL) under nitrogen. After cooling the mixture to −78° C., 2.5M nBuLi in Hexane (751 μL, 1.88 mmol) was added dropwise to the solution and the resulting mixture was stirred for 1 hour. 2.5M oxirane in THF (1.13 mL, 2.82 mmol) was added, the resulting mixture was stirred at −78° C. for 5 minutes, and then ethoxyethane-trifluoroborane (1/1) (348 μL, 2.82 mmol) was added to the mixture. The resulting mixture was stirred at −78° C. for 20 minutes and the reaction was quenched with saturated aqueous sodium bicarbonate and transferred to a separatory funnel. The mixture was extracted twice with EtOAc. Organics were washed with brine, dried over sodium sulfate, filtered and concentrated in vacuo. The crude residue was purified over silica eluting with 20-75% EtOAc/Heptane to afford 2-(5-bromo-1-methyl-1H-indazol-6-yl) ethanol (222 mg, 870 μmol). m/z (ESI-pos) M+1=255.0/257.0

### Preparation 11—Synthetic Scheme

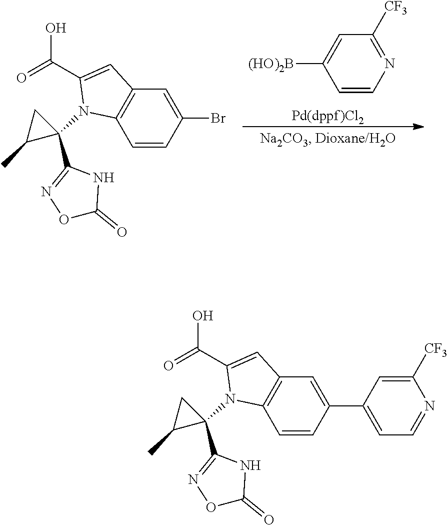

### Preparation 11—Synthetic Procedure

Step A: A solution of 5-bromo-1-[(1S,2S)-2-methyl-1-(5-oxo-4,5-dihydro-1,2,4-oxadiazol-3-yl)cyclopropyl]-2-indolecarboxylic acid (0.2 g, 529 μmol) Pd(dppf)Cl₂ (21.6 mg, 26.4 μmol), [2-(trifluoromethyl)-4-pyridyl]boranediol (151 mg, 793 μmol) and disodium carbonate (280 mg, 2.64 mmol) in 1,4-dioxane (5.29 mL) and water (1.76 mL) was heated to 80° C. for 2 hours. The solution was partitioned between EtOAc and IN aqueous HCl, the organic layer was collected and dried with Na₂SO₄, filtered and concentrated. The crude residue was purified by reverse-phase chromatography eluting with 10-75% MeCN/water with 0.1% formic acid. Desired fractions were lyophilized to afford 1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolecarboxylic acid (110 mg, 248 μmol). m/z (ESI-pos) M+1=445.2

### Preparation 12—Synthetic Scheme

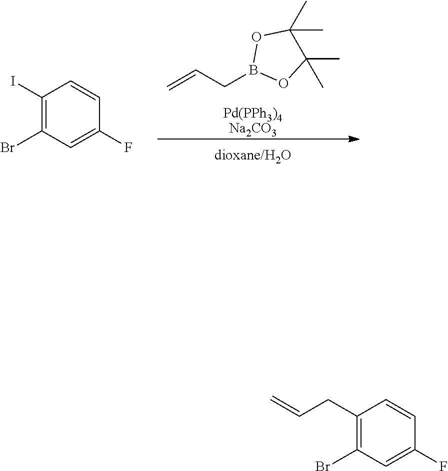

### Preparation 12—Synthetic Procedure

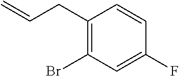

Step A: To a mixture of 2-bromo-4-fluoro-1-iodobenzene (343 mg, 1.14 mmol), disodium carbonate (242 mg, 2.28 mmol), water (1.14 mL), 1,4-dioxane (5.7 mL) was added palladium-triphenylphosphine (1/4) (132 mg, 114 μmol) and 2-allyl-4,4,5,5-tetramethyl-1,3,2-dioxaborolane (641 μL, 3.42 mmol) at room temperature. The mixture was sparged with N₂ for 10 minutes and stirred at 80° C. overnight. Additional palladium-triphenylphosphine (1/4) (132 mg, 114 μmol) and 2-allyl-4,4,5,5-tetramethyl-1,3,2-dioxaborolane (641 μL, 3.42 mmol) were added and the mixture was sparged with N₂ for 10 minutes. The reaction was stirred at 80° C. for 5 hours. Additional palladium-triphenylphosphine (1/4) (66 mg, 57 μmol) and disodium carbonate (121 mg, 1.14 mmol) were added and the mixture was sparged with N₂ for 10 minutes. The reaction was stirred at 80° C. for 2 hours. The reaction mixture was allowed to cool to room temperature, diluted with water, and extracted with ethyl acetate. The organic layer was washed with brine, dried over anhydrous Na₂SO₄, and concentrated in vacuo. The crude residue was purified by column chromatography (silica, 0-20% EtOAc/heptane) to afford 1-allyl-2-bromo-4-fluorobenzene (156 mg, 726 μmol). ¹H NMR (400 MHz, CHLOROFORM-D) δ 7.30 (dd, J=8.3, 2.7 Hz, 1H), 7.19 (dd, J=8.6, 6.0 Hz, 1H), 6.98 (td, J=8.3, 2.7 Hz, 1H), 5.94 (ddt, J=16.7, 10.1, 6.5 Hz, 1H), 5.17-4.99 (m, 2H), 3.47 (d, J=6.4 Hz, 2H).

### Preparation 13—Synthetic Scheme

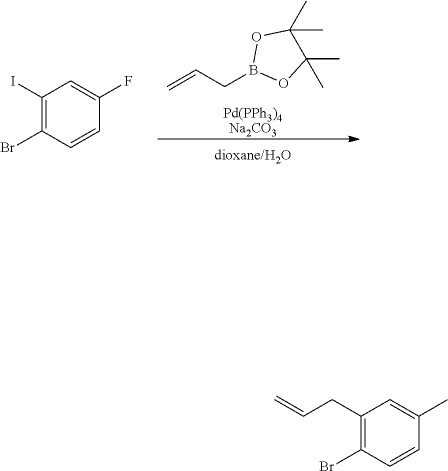

### Preparation 13—Synthetic Procedure

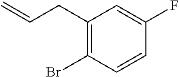

Step A: To a mixture of 1-bromo-4-fluoro-2-iodobenzene (324 mg, 1.08 mmol), disodium carbonate (228 mg, 2.15 mmol), water (1.08 mL), 1,4-dioxane (5.38 mL) was added palladium-triphenylphosphine (1/4) (124 mg, 108 μmol) and 2-allyl-4,4,5,5-tetramethyl-1,3,2-dioxaborolane (606 μL, 3.23 mmol) at room temperature. The mixture was sparged with N₂ for 10 minutes and stirred at 80° C. overnight. Additional 2-allyl-4,4,5,5-tetramethyl-1,3,2-dioxaborolane (606 μL, 3.23 mmol) and palladium-triphenylphosphine (1/4) (124 mg, 108 μmol) were added and the mixture was sparged with N₂ for 10 minutes. The mixture was stirred at 80° C. for 4 hours. Additional 2-allyl-4,4,5,5-tetramethyl-1,3,2-dioxaborolane (303 μL, 162 mmol), palladium-triphenylphosphine (1/4) (62 mg, 54 μmol), and disodium carbonate (114 mg, 1.08 mmol) were added, and the mixture was sparged with N₂ for 10 minutes. The reaction was stirred at 80° C. for 2 hours. The reaction mixture was allowed to cool to room temperature, diluted with water, and extracted with ethyl acetate. The organic layer was washed with brine, dried over anhydrous Na₂SO₄, filtered, and the filtrate was evaporated under reduced pressure to give the crude compound. The crude material was purified by column chromatography (silica, 0-20% EtOAc/heptane) to afford 2-allyl-1-bromo-4-fluorobenzene (113 mg, 525 μmol). ¹H NMR (400 MHz, CHLOROFORM-D) δ 7.49 (dd, J=8.8, 5.4 Hz, 1H), 6.97 (dd, J=9.4, 3.1 Hz, 1H), 6.82 (td, J=8.3, 3.1 Hz, 1H), 5.94 (ddt, J=16.8, 10.1, 6.6 Hz, 1H), 5.21-5.07 (m, 2H), 3.48 (dt, J=6.6, 1.6 Hz, 2H).

### Preparation 14—Synthetic Scheme

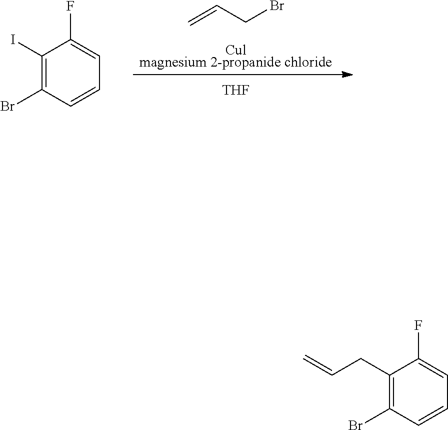

### Preparation 14—Synthetic Procedure

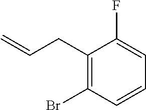

Step A: 2M magnesium 2-propanide chloride (0.65 mL, 1.3 mmol) was added to a solution of 1-bromo-3-fluoro-2-iodobenzene (0.3 g, 997 μmol) in THF (1 mL) at −20° C. The solution was stirred for 15 minutes, and copper iodide (47.5 mg, 249 μmol) was added. The solution was stirred for 10 minutes and allowed to warm to 0° C. 3-bromopropene (172 μL, 1.99 mmol) was added and the reaction was stirred at 0° C. for 30 minutes. The reaction was quenched with sat. aq. NH₄Cl, diluted with water, and extracted with ethyl acetate. The organic layer was washed with brine, dried over anhydrous Na₂SO₄, filtered, and the filtrate was evaporated under reduced pressure to give the crude compound. The crude material was purified by column chromatography (silica, 0-10% EtOAc/heptane) to afford 2-allyl-1-bromo-3-fluorobenzene (117 mg, 544 μmol). ¹H NMR (400 MHz, CHLOROFORM-D) δ 7.36 (dt, J=7.8, 1.2 Hz, 1H), 7.12-6.97 (m, 2H), 5.93 (ddt, J=16.6, 10.3, 6.1 Hz, 1H), 5.12-5.05 (m, 2H), 3.58 (dq, J=5.9, 1.8 Hz, 2H).

### Example 1—Synthesis of Compound 1

### Synthetic Scheme

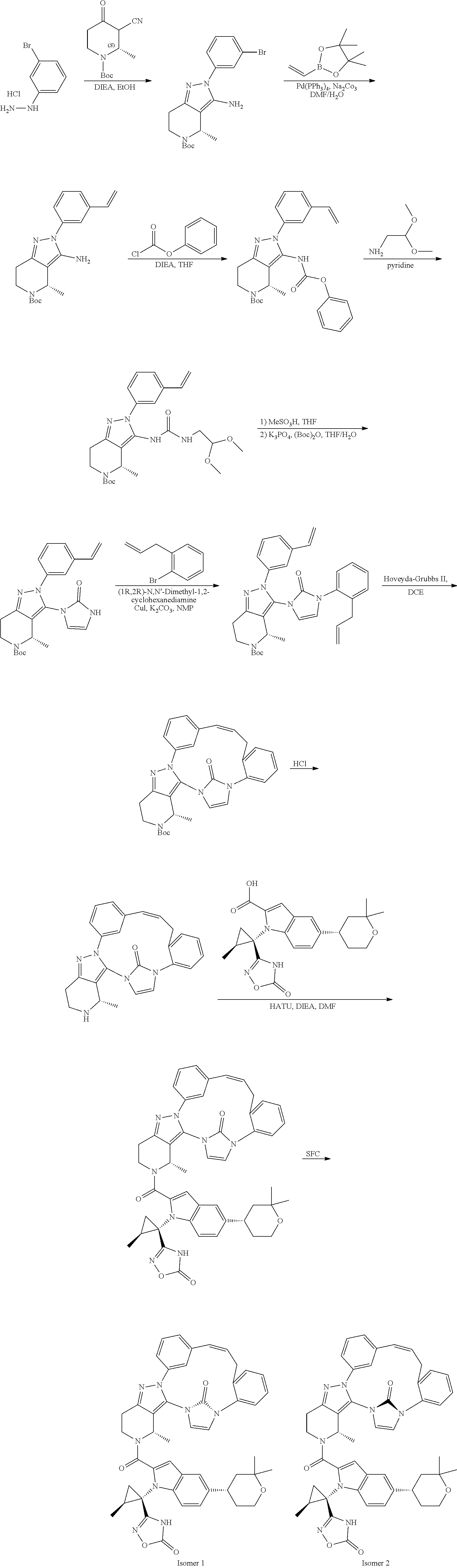

### Procedure for Synthesis of Compound 1

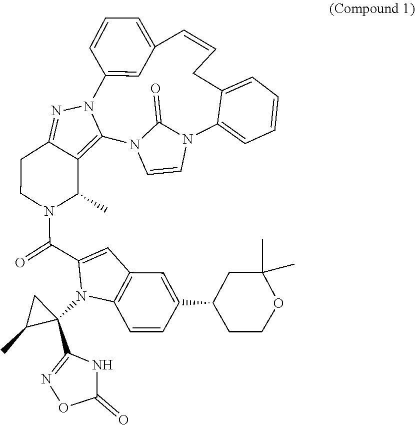

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴,0²,¹⁰,0⁴,⁹.0¹⁵,²⁰]nonacosa-1(28),3,9,12,15,17,19,22,24,26-decaen-29-one

Step A: To a mixture of (3-bromophenyl)hydrazine hydrogen chloride (2.0 g, 9.0 mmol) in ethanol (25 mL) was added DIEA (3.48 g, 27.03 mmol) at 25° C. and stirred for 5 minutes, then tert-butyl(2S)-3-cyano-2-methyl-4-oxo-1-piperidinecarboxylate (3.2 g, 13.5 mmol) was added to the mixture. The resulting mixture was stirred at 80° C. for 3 hr. The reaction mixture was cooled down to room temperature, diluted with ethyl acetate (40 mL) and washed with water (50 mL×3). The aqueous layer was extracted with ethyl acetate (40 mL×2). The combined organic layers were washed with brine solution (40 mL), dried over anhydrous Na₂SO₄, filtered and the filtrate was evaporated under reduced pressure to give the crude compound. The crude compound was purified by column chromatography (0-60% EtOAc in PE) to give tert-butyl (S)-3-amino-2-(3-bromophenyl)-4-methyl-2,4,6,7-tetrahydro-5H-pyrazolo[4,3-c]pyridine-5-carboxylate (2.0 g, 4.03 mmol) as a yellow solid. m/z (ESI-pos) M+1=407.1, 409.1.

Step B: To a mixture of tert-butyl (S)-3-amino-2-(3-bromophenyl)-4-methyl-2,4,6,7-tetrahydro-5H-pyrazolo[4,3-c]pyridine-5-carboxylate (1.22 g, 3.0 mmol), 4,4,5,5-tetramethyl-2-vinyl-1,3,2-dioxaborolane (2.31 g, 15.0 mmol) and Na₂CO₃ (636 mg, 6.0 mmol) in DMF/H₂O (15 mL/3 mL) was added Pd(PPh₃)₄ (346 mg, 0.3 mmol) at 25° C. under a N₂ atmosphere. The resulting mixture was stirred at 80° C. for 16 hr. The reaction mixture was cooled down to room temperature, diluted with ethyl acetate (50 mL) and washed with water (50 mL×3). The aqueous layer was extracted with ethyl acetate (30 mL×3). The combined organic layers were washed with brine solution (50 mL×3), dried over anhydrous Na₂SO₄, filtered and concentrated under reduced pressure to give the crude compound. The crude compound was purified by column chromatography (0-50% EtOAc in PE) to give tert-butyl (S)-3-amino-4-methyl-2-(3-vinylphenyl)-2,4,6,7-tetrahydro-5H-pyrazolo[4,3-c]pyridine-5-carboxylate (845 mg, 1.90 mmol) as a yellow oil. m/z (ESI-pos) M+1=355.2

Step C: To a solution of tert-butyl (S)-3-amino-4-methyl-2-(3-vinylphenyl)-2,4,6,7-tetrahydro-5H-pyrazolo[4,3-c]pyridine-5-carboxylate (845 mg, 2.38 mmol), N-ethyldiisopropylamine (921 mg, 7.14 mmol) in Tetrahydrofuran (15 mL) was added benzenyl chloroformate (742 mg, 4.76 mmol). The reaction mixture was stirred at 25° C. for 2 hr. The reaction mixture was concentrated to give the crude tert-butyl (S)-4-methyl-3-((phenoxycarbonyl)amino)-2-(3-vinylphenyl)-2,4,6,7-tetrahydro-5H-pyrazolo[4,3-c]pyridine-5-carboxylate (1.02 g crude) as a yellow oil, which was used directly in the next step. m/z (ESI-pos) M+1=475.1

Step D: Crude tert-butyl (S)-4-methyl-3-((phenoxycarbonyl)amino)-2-(3-vinylphenyl)-2,4,6,7-tetrahydro-5H-pyrazolo[4,3-c]pyridine-5-carboxylate (1.02 g, 2.15 mmol) was dissolved in pyridine (15 mL), to which 2,2-dimethoxyethylamine (226 mg, 2.15 mmol) was added. The mixture was stirred at 25° C. for 3 hr. The reaction mixture was concentrated to give the crude tert-butyl (S)-3-(3-(2,2-dimethoxyethyl)ureido)-4-methyl-2-(3-vinylphenyl)-2,4,6,7-tetrahydro-5H-pyrazolo[4,3-c]pyridine-5-carboxylate (1.16 g crude) as a yellow oil, which was directly used for next step. m/z (ESI-pos) M+1=486.3

Step E: To a solution of crude tert-butyl (S)-3-(3-(2,2-dimethoxyethyl)ureido)-4-methyl-2-(3-vinylphenyl)-2,4,6,7-tetrahydro-5H-pyrazolo[4,3-c]pyridine-5-carboxylate (1.16 g, 2.39 mmol) in tetrahydrofuran (20 mL) was treated with methanesulfonic acid (1.14 g, 11.95 mmol). The mixture was stirred at 60° C. for 2 hr. Aqueous K₃PO₄ was added to adjusted pH to about 9, and then tert-butoxycarbonyl-tert-butyl carbonate (781 mg, 3.58 mmol) was added, and the resulting mixture was stirred at 25° C. for 16 h. The reaction mixture was diluted with ethyl acetate (50 mL, washed with water (30 mL×2) and the aqueous layer was extracted with ethyl acetate (30 mL×2). The combined organic layers were washed with brine solution (50 mL) and dried over anhydrous Na₂SO₄, filtered and the filtrate was evaporated under reduced pressure to give the crude compound. The crude compound was purified by column chromatography (0-60% EtOAc in PE) to give tert-butyl (S)-4-methyl-3-(2-oxo-2,3-dihydro-1H-imidazol-1-yl)-2-(3-vinylphenyl)-2,4,6,7-tetrahydro-5H-pyrazolo[4,3-c]pyridine-5-carboxylate (500 mg, 1.06 mmol) as a yellow solid. M/z (ESI-pos) M+1=422.2

Step F: To a solution of tert-butyl (S)-4-methyl-3-(2-oxo-2,3-dihydro-1H-imidazol-1-yl)-2-(3-vinylphenyl)-2,4,6,7-tetrahydro-5H-pyrazolo[4,3-c]pyridine-5-carboxylate (500 mg, 1.18 mmol) and 1-allyl-2-bromobenzene (301 mg, 1.53 mmol) in N-methyl-2-pyrrolidone (10 mL) were added (1R,2R)-1,2-bis(methylamino)cyclohexane (84 mg, 0.59 mmol), CuI (224 mg, 1.18 μmol) and K₂CO₃ (488 mg, 3.54 mmol). The reaction mixture was heated at 130° C. for 3 hr under the N₂. The reaction mixture was cooled to ambient temperature and diluted with ethyl acetate (50 mL), washed with water (30 mL×3) and the aqueous layer was extracted with ethyl acetate (30 mL×3). The combined organic layers were washed with brine solution (50 mL), dried over anhydrous Na₂SO₄ and evaporated under reduced pressure to give the crude compound. The crude compound was purified by column chromatography (0-60% EtOAc in PE) to give tert-butyl (S)-3-(3-(2-allylphenyl)-2-oxo-2,3-dihydro-1H-imidazol-1-yl)-4-methyl-2-(3-vinylphenyl)-2,4,6,7-tetrahydro-5H-pyrazolo[4,3-c]pyridine-5-carboxylate (360 mg) as a yellow solid. m/z (ESI-pos) M+1=538.3

Step G: To a solution of tert-butyl (S)-3-(3-(2-allylphenyl)-2-oxo-2,3-dihydro-1H-imidazol-1-yl)-4-methyl-2-(3-vinylphenyl)-2,4,6,7-tetrahydro-5H-pyrazolo[4,3-c]pyridine-5-carboxylate (350 mg, 0.65 mmol) in DCE (350 mL) was added Hoveyda-Grubbs II (350 mg, 0.56 mmol) at 25° C. under N₂ protection. The reaction mixture was heated at 80° C. for 3 hr under the N₂. The reaction mixture was cooled to ambient temperature and quenched with water (300 mL) and the aqueous layer was extracted with dichloromethane (50 mL×3). The combined organic layers were washed with brine solution (50 mL), dried over anhydrous Na₂SO₄, filtered and concentrated to give the crude compound. The crude compound was purified by column chromatography (0-100% EtOAc in PE) to give tert-butyl (S)-8-methyl-29-oxo-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,22,24,26-decaene-7-carboxylate (248 mg) as a yellow solid. m/z (ESI-pos) M+1=510.2

Step H: tert-butyl (S)-8-methyl-29-oxo-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,22,24,26-decaene-7-carboxylate (80 mg, 0.15 mmol) was dissolved in HCl/1,4-dioxane (4M, 4 mL) and stirred at 25° C. for 1 h. The reaction mixture was directly concentrated under reduced pressure to give (S)-8-methyl-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,22,24,26-decaen-29-one hydrogen chloride (100 mg, 0.24 mmol) as a yellow solid. m/z (ESI-pos) M+1=410.1

Step I: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (65 mg, 0.157 mmol), in dimethylformamide (3 mL) was added DIEA (60 mg, 0.471 mmol) and HATU (89 mg, 0.235 mmol), the mixture stirred at rt for 10 min, then(S)-8-methyl-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,22,24,26-decaen-29-one hydrogen chloride (100 mg crude, 0.157 mmol) was added to the mixture. The mixture was stirred at ambient temperature overnight. The reaction mixture was diluted with ethyl acetate (20 mL), washed with water (20 mL×2). The aqueous layer was extracted with ethyl acetate (20 mL×2). The combined organic layers were washed with brine solution (20 mL), dried over anhydrous Na₂SO₄, filtered and the filtrate was evaporated under reduced pressure to give the crude compound and purified by column chromatography (0-10% MeOH in DCM) to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,22,24,26-decaen-29-one (90 mg) as a yellow solid.

Step J: (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,22,24,26-decaen-29-one (90 mg, 0.11 mmol) was dissolved in MeOH (150 mL) and separated by chiral supercritical fluid chromatography (separation condition: Daicel CHIRALPAK OJ_3, 3*150 mm, 3 um; Mobile Phase: CO₂/MeOH (0.1% DEA)=45/55 at 2 mL/min; Temp: 37° C.; Wavelength: 254 nm) to afford the crude Isomer 1 (30 mg, purity: 90%) and Isomer 2 (30 mg, purity: 90%), which was further purified by pre-HPLC [Waters MS-triggered Prep-LC with SQD2 detector, Xbridge 5 u C18 150×19 mm, eluted with 60-100% CAN in H₂O (0.1% FA)] to give pure Isomer 1 (11.6 mg) and Isomer 2 (10.1 mg) as white solids (The absolute configuration was unknown, the configuration of two isomers were assigned arbitrarily).

Isomer 1: m/z (ESI-pos) M+1=803.3; ¹H NMR (400 MHz, DMSO) δ 11.63 (s, 1H) 7.63-7.57 (m, 2H), 7.51-7.45 (m, 3H), 7.39-7.33 (m, 3H), 7.24 (d, J=8.4 Hz, 1H), 7.15 (d, J=7.6 Hz, 1H), 6.97 (s, 1H), 6.83 (s, 1H), 6.72-6.65 (m, 2H), 6.57 (d, J=11.2 Hz, 1H), 5.96-5.90 (m, 1H), 5.64 (s, 1H), 4.43 (s, 1H), 3.73-3.71 (m, 2H), 3.57 (s, 1H), 3.26 (d, J=8.0 Hz, 3H), 3.00-2.88 (m, 3H), 1.77-1.68 (m, 4H), 1.63-1.50 (m, 5H), 1.27 (s, 3H), 1.24-1.19 (m, 6H).

Isomer 2: m/z (ESI-pos) M+1=803.3; ¹H NMR (400 MHz, DMSO) δ 11.56 (s, 1H), 7.65 (d, J=7.2 Hz, 1H), 7.56-7.36 (m, 7H), 7.23 (d, J=8.4 Hz, 1H), 7.17 (d, J=7.2 Hz, 1H), 6.96 (s, 1H), 6.86 (s, 1H), 6.75-6.72 (m, 1H), 6.65 (d, J=2.4 Hz, 1H), 6.58 (d, J=11.2 Hz, 1H), 5.99-5.92 (m, 1H), 5.65 (s, 1H), 4.41 (s, 1H), 3.73-3.70 (m, 2H), 3.60-3.52 (m, 1H), 3.29-3.25 (m, 3H), 2.95-2.86 (m, 3H), 1.80-1.68 (m, 4H), 1.61-1.49 (m, 5H), 1.27 (s, 3H), 1.22-1.12 (m, 6H). (The absolute configuration was unknown, the configuration was assigned arbitrarily)

### Example 2—Synthesis of Compound 2

### Synthetic Scheme

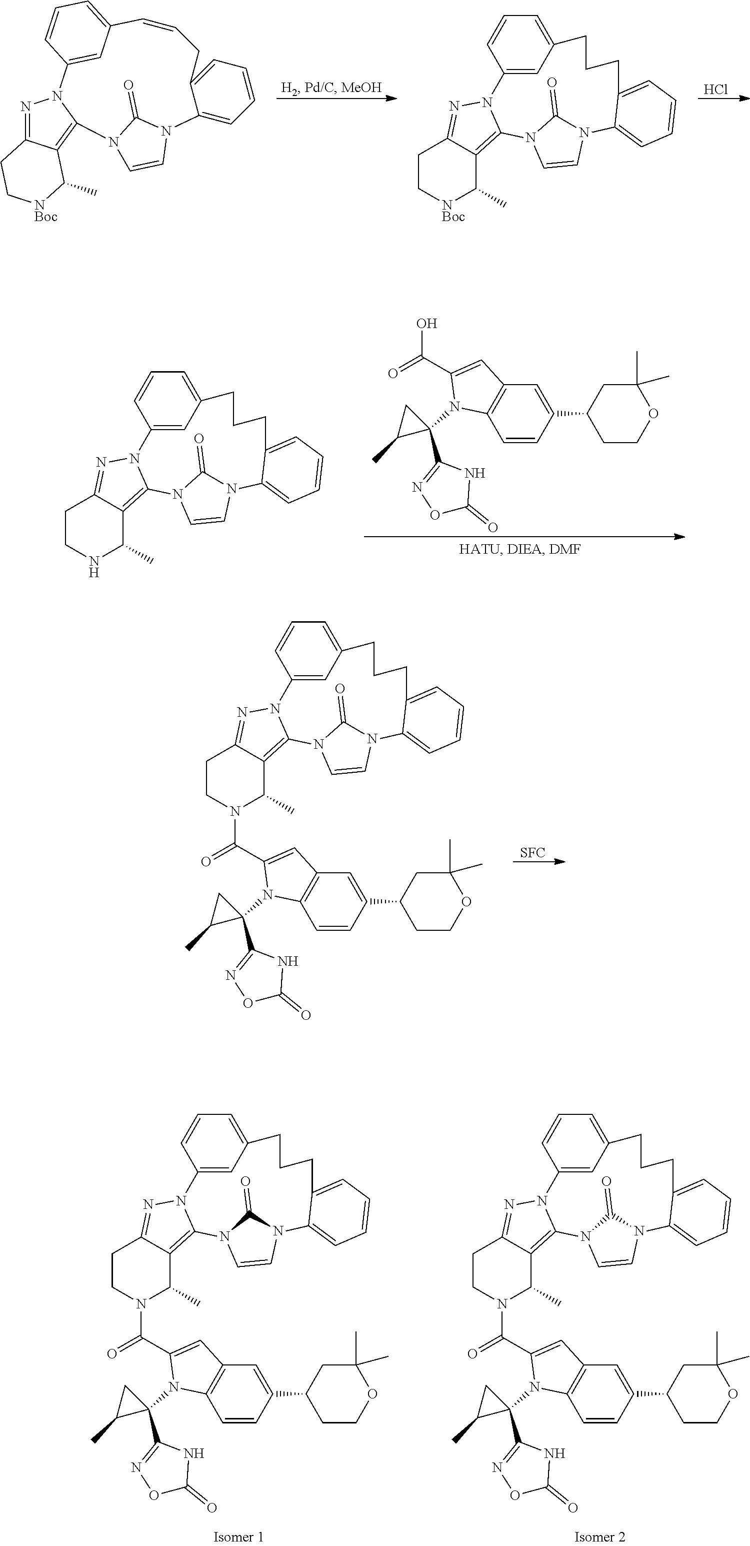

### Procedure for Synthesis of Compound 2

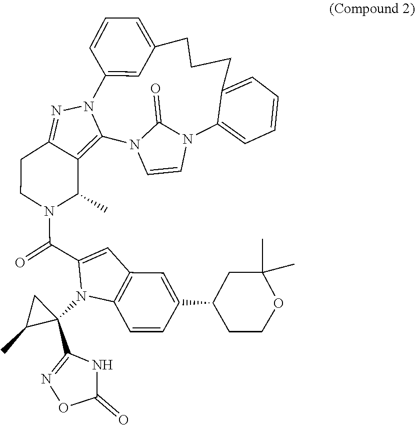

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰,0⁴,⁹,0¹⁵,²⁰]nonacosa-1(28),3,9,12,15,17,19,24,26-nonaen-29-one

Step A: To a solution of tert-butyl (S)-8-methyl-29-oxo-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,22,24,26-decaene-7-carboxylate (70 mg, 0.13 mmol, from Example 1, step G) in methanol (8 mL) was added Pd/C (35 mg, 50% w/w) at 25° C. The mixture was evacuated and backfilled with H₂ (repeat three times). The mixture was stirred at 25° C. under H₂ balloon for 16 h. The reaction mixture was filtered and the filtrate was evaporated under reduced pressure to give the tert-butyl(S)-8-methyl-29-oxo-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaene-7-carboxylate (70 mg) as a yellow solid. m/z (ESI-pos) M+1=512.2

Step B: tert-butyl (S)-8-methyl-29-oxo-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaene-7-carboxylate (70 mg, 0.14 mmol) was dissolved in HCl/1,4-dioxane (4M, 4 mL) and stirred at 25° C. for 1 h. The reaction mixture was directly concentrated under reduced pressure to give crude(S)-8-methyl-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (80 mg, 0.19 mmol) as a yellow solid. m/z (ESI-pos) M+1=412.2

Step C: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (80 mg, 0.194 mmol) in DMF (3 mL) was added DIPEA (75 mg, 0.582 mmol) and HATU (110 mg, 0.291 mmol), the mixture stirred at ambient temperature for 10 min, then(S)-8-methyl-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (80 mg crude, 0.19 mmol) was added to the mixture. The mixture was stirred at ambient temperature overnight. The reaction mixture was diluted with ethyl acetate, washed with water (2×). The aqueous layer was extracted with ethyl acetate (2×). The combined organic layers were washed with brine, dried over anhydrous Na₂SO₄, filtered and the filtrate was evaporated under reduced pressure to give the crude compound and purified by column chromatography (0-10% MeOH in DCM) to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one (60 mg) as a yellow solid. m/z (ESI-pos) M+1=805.3

Step D: (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one (60 mg, 0.07 mmol) was dissolved in MeOH (150 mL) and separated by chiral supercritical fluid chromatography (separation condition: Daicel CHIRALPAK IA 4.6*250 mm, 5 um; Mobile Phase: CO₂/EtOH (0.1% DEA)=50/50 at 1.5 mL/min; Temp: 37° C.; Wavelength: 254 nm) to afford the Isomer 1 (30 mg) and Isomer 2 (30 mg), which was further purified by pre-HPLC [Instrument: Waters MS-triggered Prep-LC with SQD2 detector; Column: Xbridge 5 u C18 150×19 mm; Gradient: eluted with 60-100% ACN in H₂O (0.1% FA)] to give Isomer 1 (10.5 mg) and Isomer 2 (12.2 mg) as white solids.

Isomer 1: m/z (ESI-pos) M+1=805.3; ¹H NMR (400 MHz, DMSO) δ 11.59 (s, 1H), 7.50 (s, 1H), 7.43-7.38 (m, 6H), 7.35-7.27 (m, 1H), 7.24-7.19 (m, 2H), 7.15 (s, 1H), 6.88 (s, 1H), 6.60-6.55 (m, 2H), 5.60 (s, 1H), 4.43 (s, 1H), 3.72-3.70 (m, 2H), 3.62-3.55 (m, 1H), 3.02-2.99 (m, 2H), 2.88-2.83 (m, 2H), 2.75-2.68 (m, 1H), 2.37-2.29 (m, 1H), 2.15-2.08 (m, 1H), 1.91-1.86 (m, 2H), 1.70-1.67 (m, 3H), 1.61-1.49 (m, 6H), 1.31-1.24 (m, 4H), 1.18 (s, 6H).

Isomer 2: m/z (ESI-pos) M+1=805.3; ¹H NMR (400 MHz, DMSO) δ 11.63 (s, 1H), 7.51 (s, 1H), 7.47-7.39 (m, 5H), 7.36-7.28 (m, 2H), 7.25-7.20 (m, 2H), 7.13 (s, 1H), 6.84 (s, 1H), 6.62 (s, 2H), 5.61 (s, 1H), 4.44 (s, 1H), 3.73 (d, J=7.6 Hz, 2H), 3.63-3.52 (m, 1H), 2.91-2.73 (m, 5H), 2.37-2.31 (m, 1H), 2.12-2.07 (m, 1H), 1.91-1.85 (m, 3H), 1.72-1.69 (m, 3H), 1.65-1.50 (m, 6H), 1.27 (s, 3H), 1.19 (s, 6H). (The absolute configuration was unknown, the configuration was assigned arbitrarily)

### Example 3—Synthesis of Compound 3

### Synthetic Scheme

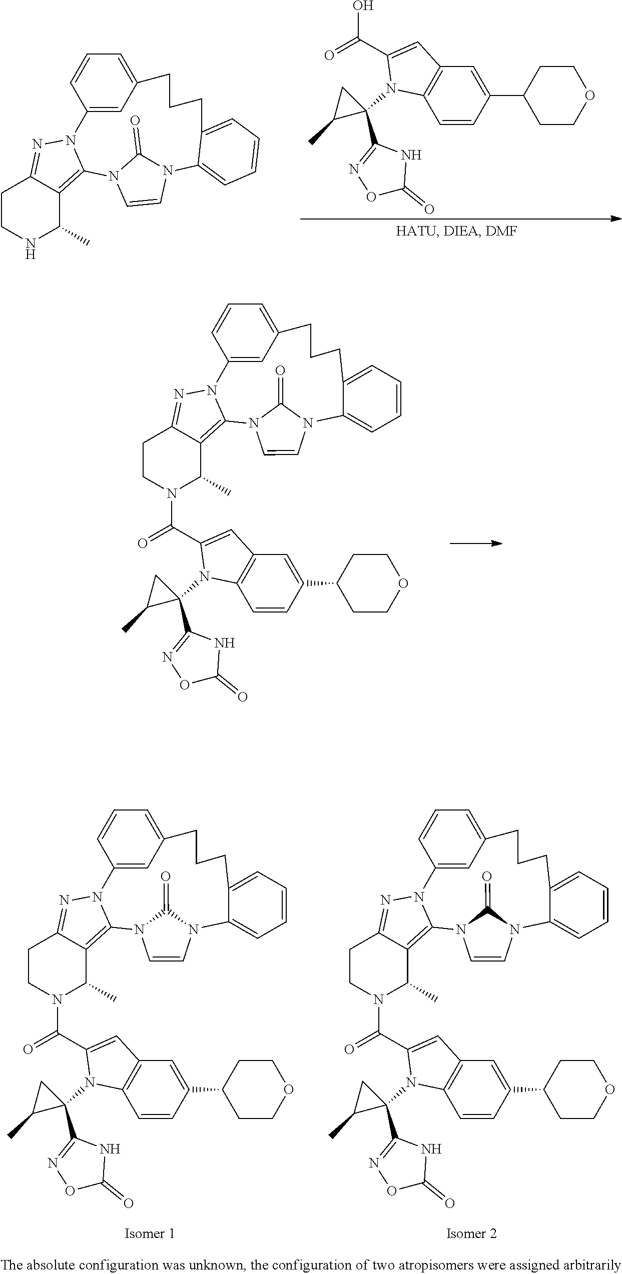

### Procedure for Synthesis of Compound 3

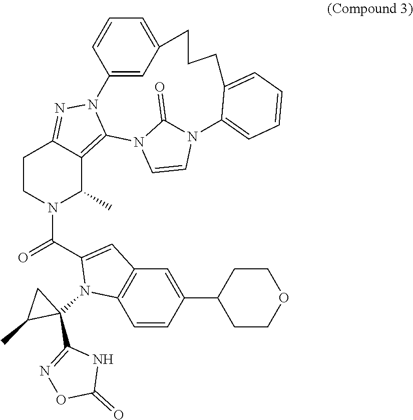

### (S)-8-methyl-7-({1-[(1S,2S)-2-methyl-1-(5-oxo-4,5-dihydro-1,2,4-oxadiazol-3-yl)cyclopropyl]-5-(tetrahydro-4H-pyran-4-yl)-2-indolyl}carbonyl)-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (466 mg, 1.22 mmol) in dimethylformamide (8 mL) was added DIEA (1.57 g, 12.2 mmol) and HATU (693 mg, 1.82 mmol) at 25° C., the mixture stirred for 10 min, then(S)-8-methyl-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (500 mg crude, 1.22 mmol) was added to the mixture. The mixture was stirred at ambient temperature overnight. The reaction mixture was diluted with ethyl acetate, washed with water (3×). The aqueous layer was extracted with ethyl acetate (3×). The combined organic layers were washed with brine solution, dried over anhydrous Na₂SO₄, filtered and the filtrate was evaporated under reduced pressure to give the crude compound and purified by pre-HPLC [Instrument: GILSON Prep LC with UV detector; Column: XBridge Prep C18 19*150 mm 5 μm; Gradient: eluted with 70-95% ACN in H2O (0.1% FA) in 10 min; Flow rate: 15 ml/min] to give (S)-8-methyl-7-({1-[(1S,2S)-2-methyl-1-(5-oxo-4,5-dihydro-1,2,4-oxadiazol-3-yl)cyclopropyl]-5-(tetrahydro-4H-pyran-4-yl)-2-indolyl}carbonyl)-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one (225 mg) as a yellow solid

Step B: (S)-8-methyl-7-({1-[(1S,2S)-2-methyl-1-(5-oxo-4,5-dihydro-1,2,4-oxadiazol-3-yl)cyclopropyl]-5-(tetrahydro-4H-pyran-4-yl)-2-indolyl}carbonyl)-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one (225 mg, 0.11 mmol) was dissolved in MeOH (140 mL) and separated by chiral supercritical fluid chromatography [separation condition: Daicel CHIRALPAK IA 4.6*250 mm, 5 um; Mobile Phase: CO2/MeOH (0.1% DEA)=50/50 at 1.5 mL/min; Temp: 37° C.; Wavelength: 254 nm] to afford Isomer 1 (84.0 mg) and Isomer 2 (104.5 mg) as white solids (The absolute configuration was unknown, the configuration of two atropisomers were assigned arbitrarily). Isomer 1: m/z (ESI-pos) M+1=777.3; ¹H NMR (400 MHz, DMSO) δ 11.52 (s, 1H), 7.50 (s, 1H), 7.43-7.39 (m, 6H), 7.35-7.31 (m, 1H), 7.25-7.21 (m, 2H), 7.14 (s, 1H), 6.88 (s, 1H), 6.60-6.55 (m, 2H), 5.66-5.57 (m, 1H), 4.52-4.35 (m, 1H), 3.97-3.94 (m, 2H), 3.66-3.56 (m, 1H), 3.50-3.43 (m, 2H), 2.89-2.81 (m, 3H), 2.72-2.65 (m, 1H), 2.35-2.31 (m, 1H), 2.16-2.08 (m, 1H), 1.93-1.86 (m, 2H), 1.75-1.71 (m, 6H), 1.59-1.48 (m, 4H), 1.30-1.11 (m, 4H). Isomer 2: m/z (ESI-pos) M+1=777.3; ¹H NMR (400 MHz, DMSO) δ 11.59 (s, 1H), 7.52 (s, 1H), 7.42-7.39 (m, 5H), 7.38-7.33 (m, 2H), 7.28-7.24 (m, 1H), 7.21-7.19 (m, 1H), 7.13 (s, 1H), 6.90-6.81 (m, 1H), 6.70-6.55 (m, 2H), 5.71-5.57 (m, 1H), 4.56-4.30 (m, 1H), 3.97-3.95 (m, 2H), 3.50-3.44 (m, 3H), 2.86-2.75 (m, 4H), 2.35-2.31 (m, 1H), 2.13-2.06 (m, 1H), 1.91-1.71 (m, 10H), 1.59-1.48 (m, 2H), 1.28-1.13 (m, 4H).

### Example 4—Synthesis of Compound 4

### Synthetic Scheme

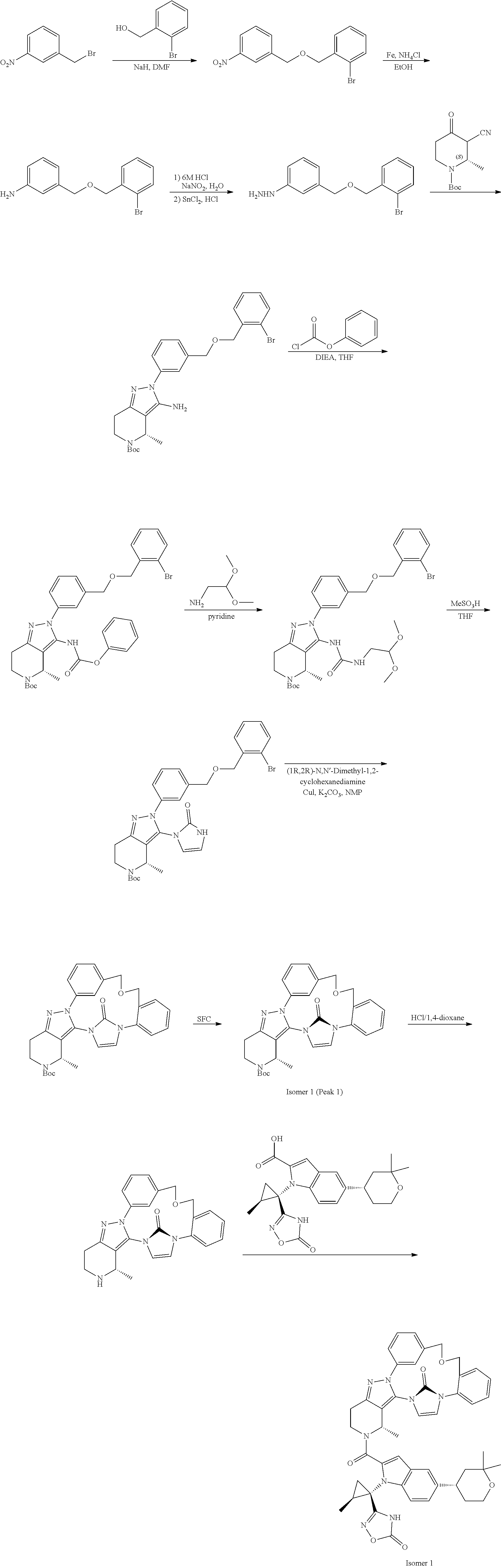

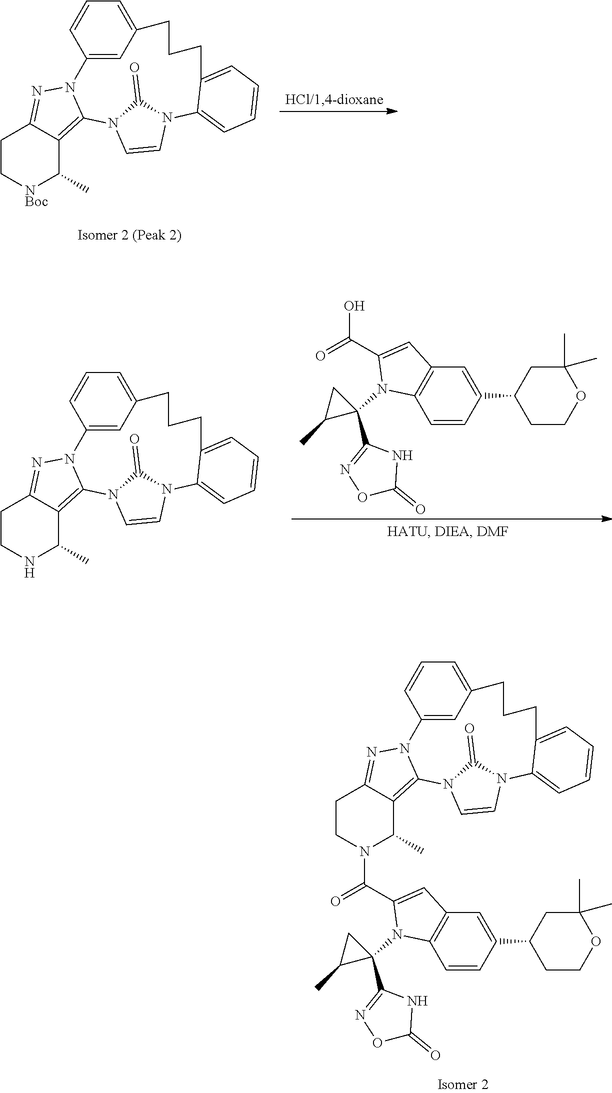

### Procedure for Synthesis of Compound 4

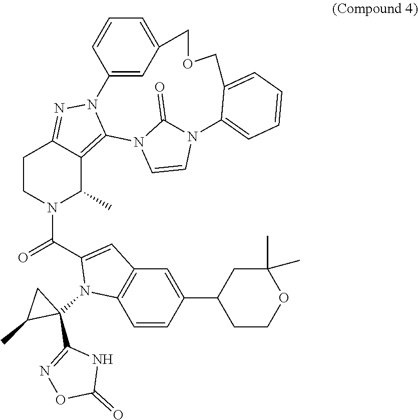

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one

Step A: To the solution of (2-bromophenyl) methanol (3 g, 16 mmol) in DMF (60 mL, 775 mmol) was added sodium hydride (770 mg, 32.1 mmol) at 0° C. under N₂. The reaction stirred at 0° C. for 30 min. Then 1-(bromomethyl)-3-nitrobenzene (4.16 g, 19.2 mmol) was added to the mixture. The reaction was allowed to warm to ambient temperature where it stirred for 2 h. The reaction mixture was quenched with saturated aqueous NH₄Cl (20 mL), diluted with ethyl acetate (30 mL), washed with water (20 mL×2) and the aqueous layer was extracted with ethyl acetate (20 mL×2). The combined organic layers were washed with brine (10 mL), dried over anhydrous Na₂SO₄ and evaporated under reduced pressure to give the crude compound, which was purified by column chromatography (0-10% EtOAc in PE) to give 1-bromo-2-(((3-nitrobenzyl)oxy)methyl)benzene (4.7 g, 14.6 mmol) as a yellow oil. ¹H NMR (400 MHz, DMSO) δ 8.24 (s, 1H), 8.17 (dd, J=1.6 Hz, 8.0 Hz, 1H), 7.85 (d, J=7.6 Hz, 1H), 7.71-7.61 (m, 2H), 7.56 (dd, J=1.2 Hz, 7.2 Hz, 1H), 7.43 (td, J=0.8 Hz, 1H), 7.28 (td, J=1.6 Hz, 1H), 4.76 (s, 2H), 4.63 (s, 2H).

Step B: To a solution of 1-bromo-2-(((3-nitrobenzyl)oxy)methyl)benzene (4.7 g, 14.6 mmol) in the ethanol (80 mL) and water (10 mL) was added iron (4.07 g, 72.9 mmol) and ammonium chloride (3.9 g, 72.9 mmol). The reaction was stirred at 80° C. for 2 h. The reaction mixture was cooled to ambient temperature and diluted with ethyl acetate (50 mL), washed with water (25 mL×2) and the aqueous layer was extracted with ethyl acetate (20 mL×2). The combined organic layers were washed with brine (20 mL), dried over anhydrous Na₂SO₄, filtered and concentrated under reduced pressure to give the crude compound. The crude compound was purified by column chromatography (0-6% MeOH in DCM) to give 3-(((2-bromobenzyl)oxy)methyl)aniline (3.7 g, 12.7 mmol) as a colorless oil. m/z (ESI-pos) M+1=292.0

Step C: To a solution of 3-(((2-bromobenzyl)oxy)methyl)aniline (3.7 g, 12.7 mmol) in 6M HCl at 0° C. was added a solution of sodium nitrite (874 mg, 12.7 mmol) in water (103 mL, 5.71 mol) dropwise at 0° C. The mixture was then stirred at 0° C. for 1 h. A solution of SnCl₂ (4.8 g, 25.3 mmol) in HCl (30 mL) was added dropwise to the reaction mixture and then the mixture was allowed to warm to ambient temperature and stirred for 2 h. The pH was adjusted to ~14 with 4N aqueous NaOH. The mixture was diluted with ethyl acetate (40 mL), washed with water (25 mL×2) and the aqueous layer was extracted with ethyl acetate (25 mL×2). The combined organic layers were washed with brine (40 mL) and dried over anhydrous Na₂SO₄, filtered and concentrated under reduced pressure. The residue was purified by column chromatography (0-10% MeOH in DCM) to give (3-(((2-bromobenzyl)oxy)methyl)phenyl)hydrazine (3 g, 9.77 mmol) as a yellow oil. m/z (ESI-pos) M+1=307.0/309.0

Step D: To a solution of (3-(((2-bromobenzyl)oxy)methyl)phenyl)hydrazine (1 g, 3.26 mmol) in Ethanol (30 mL) was added tert-butyl(2S)-3-cyano-2-methyl-4-oxo-1-piperidinecarboxylate (776 mg, 3.26 mmol). The resulting mixture was stirred at 80° C. for 8 hr. The reaction mixture was concentrated under reduced pressure to give the crude compound, which was purified by column chromatography (0-6% MeOH in DCM) to give tert-butyl (S)-3-amino-2-(3-(((2-bromobenzyl)oxy)methyl)phenyl)-4-methyl-2,4,6,7-tetrahydro-5H-pyrazolo[4,3-c]pyridine-5-carboxylate (1.1 g, 2.09 mmol) as a yellow oil. m/z (ESI-pos) M+1=527.0/529.0

Step E: To a solution of tert-butyl (S)-3-amino-2-(3-(((2-bromobenzyl)oxy)methyl)phenyl)-4-methyl-2,4,6,7-tetrahydro-5H-pyrazolo[4,3-c]pyridine-5-carboxylate (1.1 g, 2.09 mmol) and N-ethyldiisopropylamine (809 mg, 6.26 mmol) in tetrahydrofuran (30 mL) was added benzenyl chloroformate (653 mg, 4.17 mmol). The reaction was stirred at 25° C. for 2 hr. The reaction mixture was concentrated to give crude tert-butyl (S)-2-(3-(((2-bromobenzyl)oxy)methyl)phenyl)-4-methyl-3-((phenoxycarbonyl)amino)-2,4,6,7-tetrahydro-5H-pyrazolo[4,3-c]pyridine-5-carboxylate (4.1 g, 1.85 mmol) as a yellow oil, which was used directly for the next step without any further purification. m/z (ESI-pos) M+1=647.0/649.1

Step F: Crude tert-butyl (S)-2-(3-(((2-bromobenzyl)oxy)methyl)phenyl)-4-methyl-3-((phenoxycarbonyl)amino)-2,4,6,7-tetrahydro-5H-pyrazolo[4,3-c]pyridine-5-carboxylate (4.1 g, 1.85 mmol) was dissolved in pyridine (50 mL), to which 2,2-dimethoxyethylamine (2 g, 19 mmol) was added. The mixture was stirred at 25° C. for 3 hr. The reaction mixture was concentrated under reduced pressure. The crude compound was purified by column chromatography (0-5% MeOH in DCM) to give tert-butyl (S)-2-(3-(((2-bromobenzyl)oxy)methyl)phenyl)-3-(3-(2,2-dimethoxyethyl)ureido)-4-methyl-2,4,6,7-tetrahydro-5H-pyrazolo[4,3-c]pyridine-5-carboxylate (1.5 g, 2.28 mmol) as a yellow oil. m/z (ESI-pos) M+1=658.2

Step G: To a solution of tert-butyl (S)-2-(3-(((2-bromobenzyl)oxy)methyl)phenyl)-3-(3-(2,2-dimethoxyethyl)ureido)-4-methyl-2,4,6,7-tetrahydro-5H-pyrazolo[4,3-c]pyridine-5-carboxylate (1.3 g, 1.98 mmol) in tetrahydrofuran (26 mL) was added methanesulfonic acid (190 mg, 1.98 mmol). The mixture was stirred at 60° C. for 2 hr. Aqueous K₃PO₄ was added to adjust the pH to about 9, and then tert-butyl-tert-butyl (oxycarbonyloxy) formylate (432 mg, 1.98 mmol) was added and the resulting mixture was stirred at 25° C. for 1 hr. The reaction mixture was diluted with ethyl acetate (50 mL) washed with water (25 mL×2) and the aqueous layer was extracted with ethyl acetate (25 mL×2). The combined organic layers were washed with 1N aqueous HCl (20 mL), brine (20 mL) and dried over anhydrous Na₂SO₄. The mixture was filtered and the filtrate was evaporated under reduced pressure to give the crude compound. The crude compound was purified by column chromatography (0-80% EA in PE) to give product tert-butyl (4S)-2-(3-(((2-bromobenzyl)oxy)methyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (970 mg, 1.63 mmol) as a yellow solid. m/z (ESI-pos) M+1=594.1

Step H: A mixture of tert-butyl (4S)-2-(3-(((2-bromobenzyl)oxy)methyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (570 mg, 959 μmol), CuI (30.5 mg, 479 μmol), (1R,2R)-1,2-bis(methylamino)cyclohexane (205 mg, 1.44 mmol) and dipotassium carbonate (398 mg, 2.88 mmol) in N-methyl-2-pyrrolidone (14 mL) was stirred at 130° C. for 4 h. The reaction mixture was cooled to ambient temperature and diluted with ethyl acetate (50 mL), washed with water (25 mL×2) and the aqueous layer was extracted with ethyl acetate (25 mL×2). The combined organic layers were washed with aq. LiCl solution (20 mL) and dried over anhydrous Na₂SO₄. The mixture was filtered and the filtrate was evaporated under reduced pressure. The obtained residue was purified by column chromatography (0-80% EtOAc in PE) to give tert-butyl (S)-8-methyl-29-oxo-22-oxa-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaene-7-carboxylate (0.1 g, 195 μmol), which was separated by SFC [Column: Daicel CHIRALCEL ID, 250 mm 30 mm I.D., 10 μmMobile phase: CO2/MeOH [0.2% NH₃ (7M Solution in MeOH)]=50/50] to provide isomer 1 (40 mg) and isomer 2 (60 mg) (The absolute configuration was unknown, and assigned arbitrarily). m/z (ESI-pos) M+1=514.1

Step I: Isomer 1 from the SFC separation (40 mg, 77.9 μmol) was dissolved in HCl/1,4-dioxane (4M, 1 mL) and stirred at 25° C. for 1 h. The reaction mixture was directly concentrated to give the crude product (S)-8-methyl-22-oxa-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (40 mg, 77.4 μmol) as a yellow solid, which was used directly in the next step. m/z (ESI-pos) M+1=414.2

Step J: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (39.8 mg, 96.7 μmol) in DMF (4 mL) was added DIPEA (37.5 mg, 290 μmol) and HATU (55.2 mg, 145 μmol), the mixture stirred at ambient temperature for 10 min, then (S)-8-methyl-22-oxa-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (40 mg, 96.7 μmol) was added to the mixture. The mixture was stirred at ambient temperature overnight. The reaction mixture was diluted with ethyl acetate (15 mL), washed with water (10 mL×2). The aqueous layer was extracted with ethyl acetate (10 mL×2). The combined organic layers were washed with brine (20 mL), dried over anhydrous Na₂SO₄, filtered and concentrated under reduced pressure to give the crude compound. The obtained residue was purified by prep-HPLC [Instrument: Waters MS-triggered Prep-LC with SQD2 detector; Column: Xbridge 5 u C18 150×19 mm; Gradient: 75-100% ACN in H₂O (0.1% FA) in 9.5 min; Flow rate: 20 mL/min] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one Isomer 1 (14.5 mg) as a white solid. m/z (ESI-pos) M+1=807.3; ¹H NMR (400 MHz, DMSO) δ 11.66 (br, 1H), 7.59-7.50 (m, 7H), 7.42 (d, J=8.4 Hz, 1H), 7.30-7.24 (m, 2H), 7.17 (s, 1H), 6.90 (s, 1H), 6.73 (d, J=2.0 Hz, 1H), 6.58 (d, J=2.8 Hz, 1H), 5.76-5.56 (m, 1H), 4.65 (dd, J1=14.4 Hz, J2=46.8 Hz, 2H), 4.51-4.35 (m, 1H), 4.18 (dd, J1=9.2 Hz, J2=22.4 Hz, 2H), 3.77-3.67 (m, 2H), 3.67-3.54 (m, 1H), 3.05-2.87 (m, 3H), 1.84-1.51 (m, 10H), 1.29 (s, 3H), 1.13 (s, 6H). (The absolute configuration was unknown, the configuration was assigned arbitrarily)

Step K: Steps I and J were repeated using Isomer 2 (60 mg, 117 μmol) in place of Isomer 1 to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[23.3.1.1¹¹,¹⁴.0²,¹⁰.0⁴,⁹.0¹⁵,²⁰]nonacosa-1(28),3,9,12,15,17,19,24,26-nonaen-29-one Isomer 2 (30.7 mg) as a white solid. m/z (ESI-pos) M+1=807.3; ¹H NMR (400 MHz, DMSO) δ 11.66 (br, 1H), 7.59-7.50 (m, 7H), 7.42 (d, J=8.4 Hz, 1H), 7.30-7.24 (m, 2H), 7.17 (s, 1H), 6.90 (s, 1H), 6.73 (d, J=2.0 Hz, 1H), 6.58 (d, J=2.8 Hz, 1H), 5.76-5.56 (m, 1H), 4.65 (dd, J1=14.4 Hz, J2=46.8 Hz, 2H), 4.51-4.35 (m, 1H), 4.18 (dd, J1=9.2 Hz, J2=22.4 Hz, 2H), 3.77-3.67 (m, 2H), 3.67-3.54 (m, 1H), 3.05-2.87 (m, 3H), 1.84-1.51 (m, 10H), 1.29 (s, 3H), 1.13 (s, 6H). (The absolute configuration was unknown, the configuration was assigned arbitrarily)

### Example 5—Synthesis of Compound 5

### Synthetic Scheme

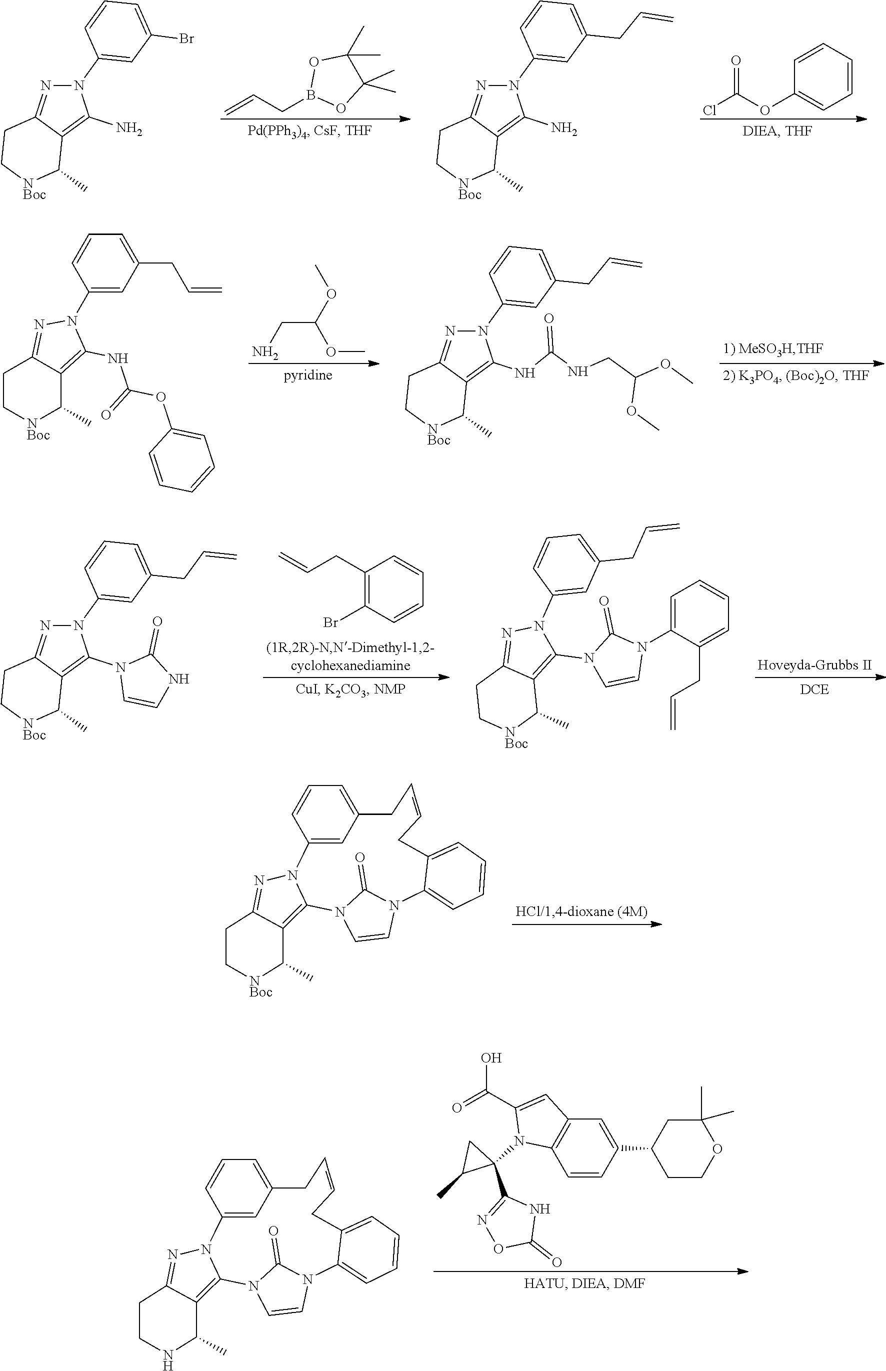

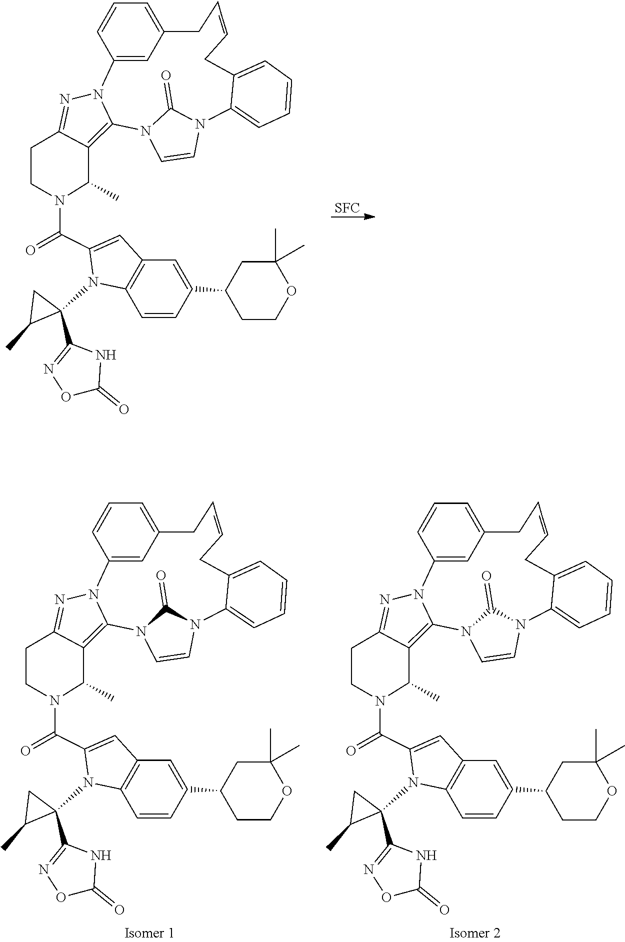

### Procedure for Synthesis of Compound 5

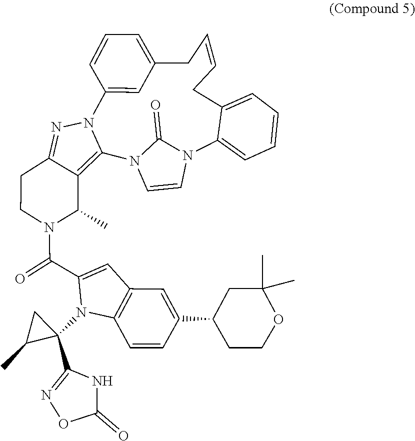

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaen-30-one

Step A: To a mixture of tert-butyl (S)-3-amino-2-(3-bromophenyl)-4-methyl-2,4,6,7-tetrahydro-5H-pyrazolo[4,3-c]pyridine-5-carboxylate (1.5 g, 3.68 mmol, from Example 1 step A), 2-allyl-4,4,5,5-tetramethyl-1,3,2-dioxaborolane (805 mg, 4.79 mmol) and CsF (1.68 g, 11.0 mmol) in THF (20 mL) was added Pd(PPh₃)₄ (213 mg, 0.184 mmol) at 25° C. under N₂ protection. The resulting mixture was stirred at 80° C. for 16 hr and then cooled down to room temperature, diluted with ethyl acetate (50 mL), washed with water (50 mL×3). The aqueous layer was extracted with ethyl acetate (30 mL×3). The combined organic layers were washed with brine (50 mL×3), dried over anhydrous Na₂SO₄, filtered and concentrated to give the crude compound. The crude compound was purified by column chromatography (0-50% EtOAc in PE) to give tert-butyl (S)-2-(3-allylphenyl)-3-amino-4-methyl-2,4,6,7-tetrahydro-5H-pyrazolo[4,3-c]pyridine-5-carboxylate (1100 mg, 2.69 mmol) as a yellow oil. m/z (ESI-pos) M+1=369.2

Step B: To a solution of tert-butyl (S)-2-(3-allylphenyl)-3-amino-4-methyl-2,4,6,7-tetrahydro-5H-pyrazolo[4,3-c]pyridine-5-carboxylate (1100 mg, 2.98 mmol), DIPEA (1156 mg, 8.94 mmol) in Tetrahydrofuran (15 mL) was added benzenyl chloroformate (929 mg, 5.96 mmol). The reaction mixture was stirred at 25° C. for 2 hr. The reaction mixture was directly concentrated under reduced pressure to give the crude tert-butyl (S)-2-(3-allylphenyl)-4-methyl-3-((phenoxycarbonyl)amino)-2,4,6,7-tetrahydro-5H-pyrazolo[4,3-c]pyridine-5-carboxylate (1.3 g crude) as a yellow oil, which was used directly in the next step without any further purification. m/z (ESI-pos) M+1=489.2

Step C: Crude tert-butyl (S)-2-(3-allylphenyl)-4-methyl-3-((phenoxycarbonyl)amino)-2,4,6,7-tetrahydro-5H-pyrazolo[4,3-c]pyridine-5-carboxylate (1.3 g, 2.66 mmol) was dissolved in pyridine (15 mL) and then 2,2-dimethoxyethylamine (279 mg, 2.66 mmol) was added. The mixture was stirred at 25° C. for 3 hr and directly concentrated under reduced pressure to give the crude residue (1.5 g) as a yellow oil, which was purified by column chromatography (0-60% EtOAc in PE) to give tert-butyl (S)-2-(3-allylphenyl)-3-(3-(2,2-dimethoxyethyl)ureido)-4-methyl-2,4,6,7-tetrahydro-5H-pyrazolo[4,3-c]pyridine-5-carboxylate (1.2 g, 2.40 mmol) as a yellow solid. m/z (ESI-pos) M+1=500.2

Step D: To a solution of tert-butyl (S)-2-(3-allylphenyl)-3-(3-(2,2-dimethoxyethyl)ureido)-4-methyl-2,4,6,7-tetrahydro-5H-pyrazolo[4,3-c]pyridine-5-carboxylate (1.2 g, 2.40 mmol) in THF (20 mL) was added methanesulfonic acid (1.15 g, 12.02 mmol). The mixture was stirred at 60° C. for 2 hr and cooled to ambient temperature. Aqueous K₃PO₄ was added to the reaction mixture to adjust pH to ~9, tert-butoxycarbonyl-tert-butyl carbonate (784 mg, 3.60 mmol) was added, and the resulting mixture was stirred at 25° C. for 16 hr. The reaction mixture was diluted with ethyl acetate (40 mL), washed with water (30 mL×2) and the aqueous layer was extracted with ethyl acetate (30 mL×2). The combined organic layers were washed with brine (50 mL) and dried over anhydrous Na₂SO₄. Filtered and concentrated under reduced pressure to give the crude compound, which was purified by column chromatography (0-60% EtOAc in PE) to give tert-butyl (S)-2-(3-allylphenyl)-4-methyl-3-(2-oxo-2,3-dihydro-1H-imidazol-1-yl)-2,4,6,7-tetrahydro-5H-pyrazolo[4,3-c]pyridine-5-carboxylate (1.0 g, 2.29 mmol) as a yellow solid. m/z (ESI-pos) M+1=436.2

Step E: To a solution of tert-butyl (S)-2-(3-allylphenyl)-4-methyl-3-(2-oxo-2,3-dihydro-1H-imidazol-1-yl)-2,4,6,7-tetrahydro-5H-pyrazolo[4,3-c]pyridine-5-carboxylate (1000 mg, 2.29 mmol) and 1-allyl-2-bromobenzene (583 mg, 2.97 mmol) in NMP (10 mL) were added (1R,2R)-1,2-bis(methylamino)cyclohexane (325 mg, 2.29 mmol), CuI (436 mg, 2.29 mmol) and K₂CO₃ (948 mg, 6.87 mmol). The reaction mixture was heated at 130° C. for 3 hr under N₂. The reaction mixture was cooled to ambient temperature and diluted with ethyl acetate (50 mL), washed with water (30 mL×3) and the aqueous layer was extracted with ethyl acetate (30 mL×3). The combined organic layers were washed with brine solution (50 mL), dried over anhydrous Na₂SO₄, filtered and evaporated under reduced pressure to give the crude compound. The crude compound was purified by column chromatography (0-80% EtOAc in PE) to give tert-butyl (S)-2-(3-allylphenyl)-3-(3-(2-allylphenyl)-2-oxo-2,3-dihydro-1H-imidazol-1-yl)-4-methyl-2,4,6,7-tetrahydro-5H-pyrazolo[4,3-c]pyridine-5-carboxylate (507 mg) as a yellow solid. m/z (ESI-pos) M+1=552.2

Step F: To a solution of tert-butyl (S)-2-(3-allylphenyl)-3-(3-(2-allylphenyl)-2-oxo-2,3-dihydro-1H-imidazol-1-yl)-4-methyl-2,4,6,7-tetrahydro-5H-pyrazolo[4,3-c]pyridine-5-carboxylate (507 mg, 0.92 mmol) in DCE (507 mL) was added Hoveyda-Grubbs II (507 mg, 0.81 mmol) at 25° C. under N₂ atmosphere. The reaction mixture was then heated at 80° C. for an additional 3 hr under N₂. The reaction mixture was then concentrated under reduced pressure to give the crude compound, which was purified by column chromatography (0-100% EtOAc in PE) to give tert-butyl (S)-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (400 mg, 83% yield) as a yellow solid. m/z (ESI-pos) M+1=524.2

Step G: tert-butyl (S)-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (190 mg, 0.36 mmol) was dissolved in HCl/1,4-dioxane (4M, 4 mL) and stirred at 25° C. for 1 h. The reaction mixture was concentrated under reduced pressure to give (S)-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaen-30-one hydrogen chloride (190 mg crude, 0.42 mmol) as a yellow solid. m/z (ESI-pos) M+1=424.2

Step H: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (110 mg, 0.26 mmol) in DMF (3 mL) were added DIEA (335 mg, 2.6 mmol) and HATU (152 mg, 0.40 mmol), the mixture stirred at ambient temperature for 10 min, then(S)-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaen-30-one hydrogen chloride (113 mg crude, 0.26 mmol) was added to the mixture. The mixture was stirred at ambient temperature overnight and then diluted with ethyl acetate (30 mL) and washed with water (30 mL×2). The aqueous layer was extracted with ethyl acetate (30 mL×3). The combined organic layers were washed with brine solution (30 mL), dried over anhydrous Na₂SO₄, filtered and concentrated under reduced pressure. The crude compound was purified by column chromatography (0-10% MeOH in DCM) to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaen-30-one (120 mg) as a yellow solid. m/z (ESI-pos) M+1=817.3

Step I: (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaen-30-one (120 mg, 0.14 mmol) was dissolved in MeOH (150 mL) and separated by chiral supercritical fluid chromatography (separation condition: Daicel CHIRALPAK IA 4.6*250 mm, 5 um; Mobile Phase: CO₂/MeOH (0.1% DEA)=50/50 at 1.5 mL/min; Temp: 37° C.; Wavelength: 254 nm) to afford the Isomer 1 (20 mg) and Isomer 2 (50 mg), which were further purified by prep-HPLC [Waters MS-triggered Pre LC with QDA detector, Welch 21.2*250 mm 10 μm C18, eluted with 60-100% ACN in H₂O (0.1% NH₃)] to give pure Isomer 1 (11.86 mg) and Isomer 2 (33.37 mg) as white solids (The absolute configuration was unknown, the configuration was assigned arbitrarily).

Isomer 1: m/z (ESI-pos) M+1=817.3; ¹H NMR (400 MHz, DMSO) δ 11.62 (s, 1H), 7.54-7.50 (m, 2H), 7.40-7.32 (m, 6H), 7.25-7.15 (m, 3H), 6.97-6.85 (m, 4H), 6.54 (d, J=3.2 Hz, 1H), 5.61-5.46 (m, 2H), 5.33-5.23 (m, 1H), 4.41 (s, 1H), 3.73-3.68 (m, 2H), 3.59-3.51 (m, 1H), 3.42-3.38 (m, 1H), 3.28-3.24 (m, 2H), 2.88-2.82 (m, 1H), 1.71-1.63 (m, 4H), 1.57-1.47 (m, 4H), 1.32-1.24 (m, 6H), 1.18 (s, 6H).

Isomer 2: m/z (ESI-pos) M+1=817.3; ¹H NMR (400 MHz, DMSO) δ 11.57 (s, 1H), 7.55-7.50 (m, 2H), 7.39-7.30 (m, 7H), 7.25-7.21 (m, 1H), 7.13 (d, J=7.6 Hz, 1H), 7.05 (s, 1H), 6.99 (s, 1H), 6.85-6.56 (m, 3H), 5.54-5.47 (m, 2H), 5.34-5.27 (m, 1H), 4.42 (s, 1H), 3.76-3.71 (m, 2H), 3.66-3.59 (m, 1H), 3.45-3.33 (m, 2H), 3.29-3.24 (m, 2H), 2.99-2.88 (m, 2H), 1.73-1.67 (m, 4H), 1.62-1.49 (m, 5H), 1.27 (s, 3H), 1.18 (s, 6H).

### Example 6—Synthesis of Compound 6

### Synthetic Scheme

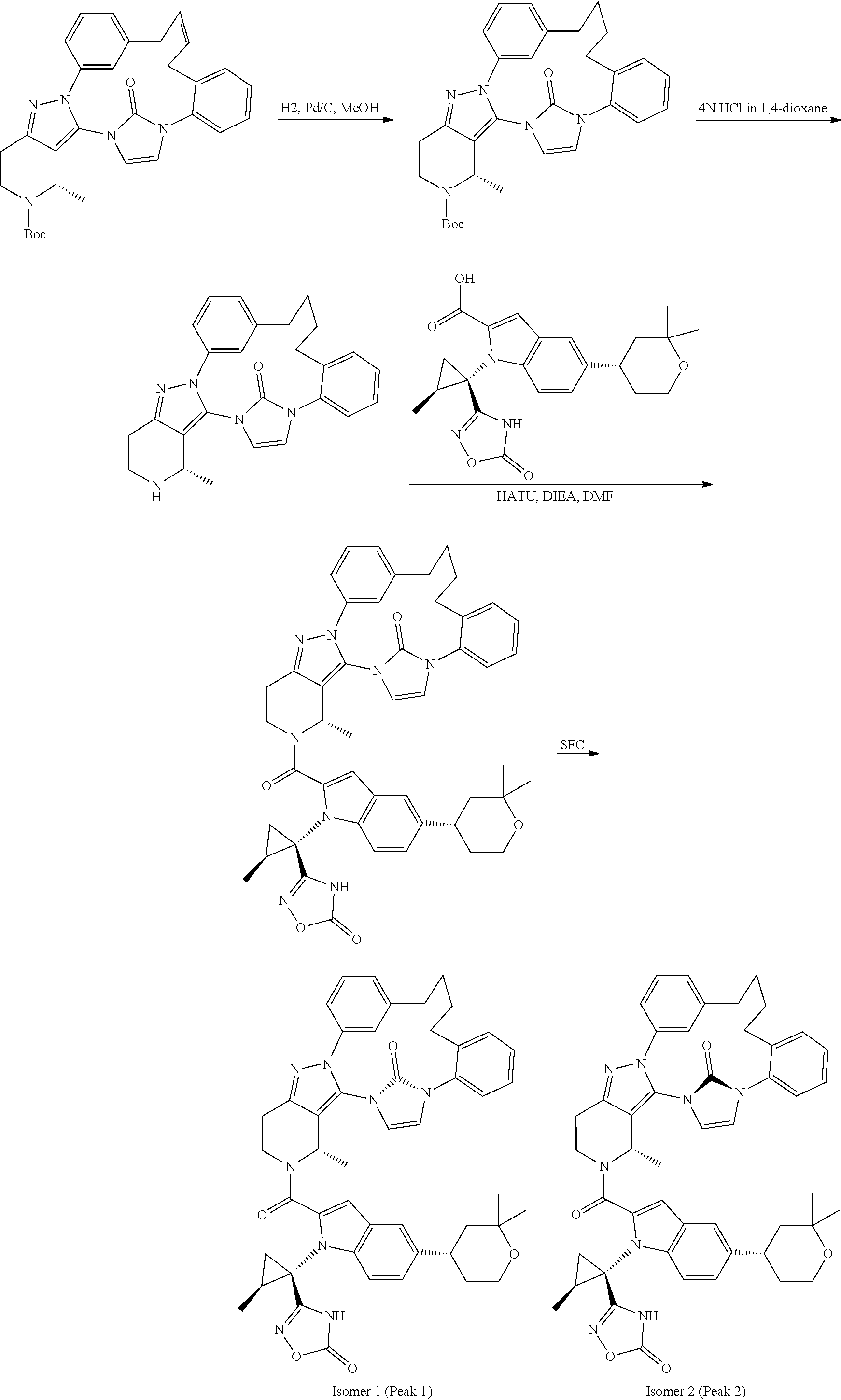

### Procedure for Synthesis of Compound 6

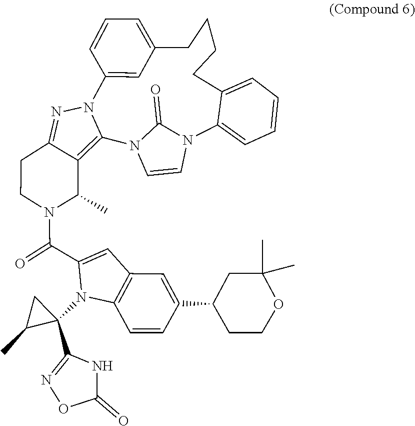

### 3-((1S,2S)-1-(5-((S)-2,2-dimethyltetrahydro-2H-pyran-4-yl)-2-((2⁴S)-2⁴-methyl-3²-oxo-2⁴,2⁵,2⁶,2⁷,3²,3³-hexahydro-2²H,3¹H-2(2,3)-pyrazolo[4,3-c]pyridina-3(1,3)-imidazola-1(1,3),4(1,2)-dibenzenacyclooctaphane-25-carbonyl)-1H-indol-1-yl)-2-methylcyclopropyl)-1,2,4-oxadiazol-5(4H)-one

Step A: To a solution of tert-butyl (S)-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (210 mg, 0.40 mmol) in methanol (10 mL) was added Pd/C (105 mg) at 25° C. followed by the introduction of a H₂ atmosphere. The mixture was stirred at 25° C. for 16 hr. The reaction mixture was then filtered and the filtrate was evaporated under reduced pressure to give tert-butyl (S)-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (150 mg, 0.28 mmol) as a yellow solid. m/z (ESI-pos) M+1=526.3

Step B: tert-butyl (S)-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (140 mg, 0.26 mmol) was dissolved in HCl/1,4-dioxane (4M, 4 mL) and stirred at 25° C. for 1 h. The reaction mixture was directly concentrated under reduced pressure to give crude(S)-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (150 mg, 0.32 mmol) as a yellow solid. m/z (ESI-pos) M+1=426.2

Step C: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (87 mg, 0.21 mmol) in DMF (3 mL) was added DIEA (114 mg, 0.88 mmol) and HATU (114 mg, 0.88 mmol), the mixture stirred at ambient temperature for 10 min, then(S)-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (75 mg crude, 0.17 mmol) was added to the mixture. The mixture was stirred at ambient temperature overnight. The reaction mixture was diluted with ethyl acetate (30 mL), washed with water (30 mL×2). The aqueous layer was extracted with ethyl acetate (30 mL×2). The combined organic layers were washed with brine (30 mL), dried over anhydrous Na₂SO₄, filtered and concentrated under reduced pressure to give the crude compound and purified by column chromatography (0-10% MeOH in DCM) to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (100 mg) as a yellow solid. m/z (ESI-pos) M+1=819.4

Step D: (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (100 mg, 0.12 mmol) was dissolved in MeOH (150 mL) and separated by chiral supercritical fluid chromatography (separation condition: Daicel CHIRALPAK IA 4.6*250 mm, 5 μm; Mobile Phase: CO2/EtOH (0.1% DEA)=50/50 at 1.5 mL/min; Temp: 37° C.; Wavelength: 254 nm) to afford crude Isomer 1(20 mg) and Isomer 2 (30 mg), which was further purified by prep-HPLC [Waters MS-triggered Prep-LC with SQD2 detector, Xbridge 5 u C18 150×19 mm, eluted with 60-100% ACN in H₂O (0.1% FA)] to give Isomer 1 (10.23 mg) and Isomer 2 (18.88 mg) as white solids (The absolute configuration was unknown, the configuration of two atropisomers were assigned arbitrarily). Isomer 1: m/z (ESI-pos) M+1=819.4; ¹H NMR (400 MHz, DMSO) δ 11.56 (s, 1H), 7.53-7.48 (m, 1H), 7.44-7.38 (m, 3H), 7.36-7.33 (m, 2H), 7.30-7.18 (m, 4H), 7.00-6.96 (m, 1H), 6.85-6.69 (m, 3H), 5.60 (s, 1H), 4.40 (s, 1H), 3.76-3.71 (m, 2H), 3.59-3.52 (m, 1H), 2.92-2.85 (m, 1H), 2.75-2.62 (m, 3H), 1.84-1.76 (m, 2H), 1.71-1.68 (m, 4H), 1.62-1.46 (m, 7H), 1.32-1.24 (m, 7H), 1.19 (s, 6H).

Isomer 2: m/z (ESI-pos) M+1=819.4; ¹H NMR (400 MHz, DMSO) δ 11.41 (s, 1H), 7.55-7.50 (m, 1H), 7.47-7.40 (m, 3H), 7.38-7.35 (m, 2H), 7.29-7.20 (m, 4H), 7.02-6.98 (m, 1H), 6.90-6.70 (m, 3H), 5.61 (s, 1H), 4.40 (s, 1H), 3.74-3.72 (m, 2H), 3.62-3.47 (m, 1H), 2.95-2.85 (m, 1H), 2.75-2.63 (m, 3H), 1.85-1.77 (m, 2H), 1.73-1.70 (m, 4H), 1.67-1.41 (m, 7H), 1.32-1.26 (m, 7H), 1.20 (s, 6H)

### Example 7—Synthesis of Compound 7

### Synthetic Scheme

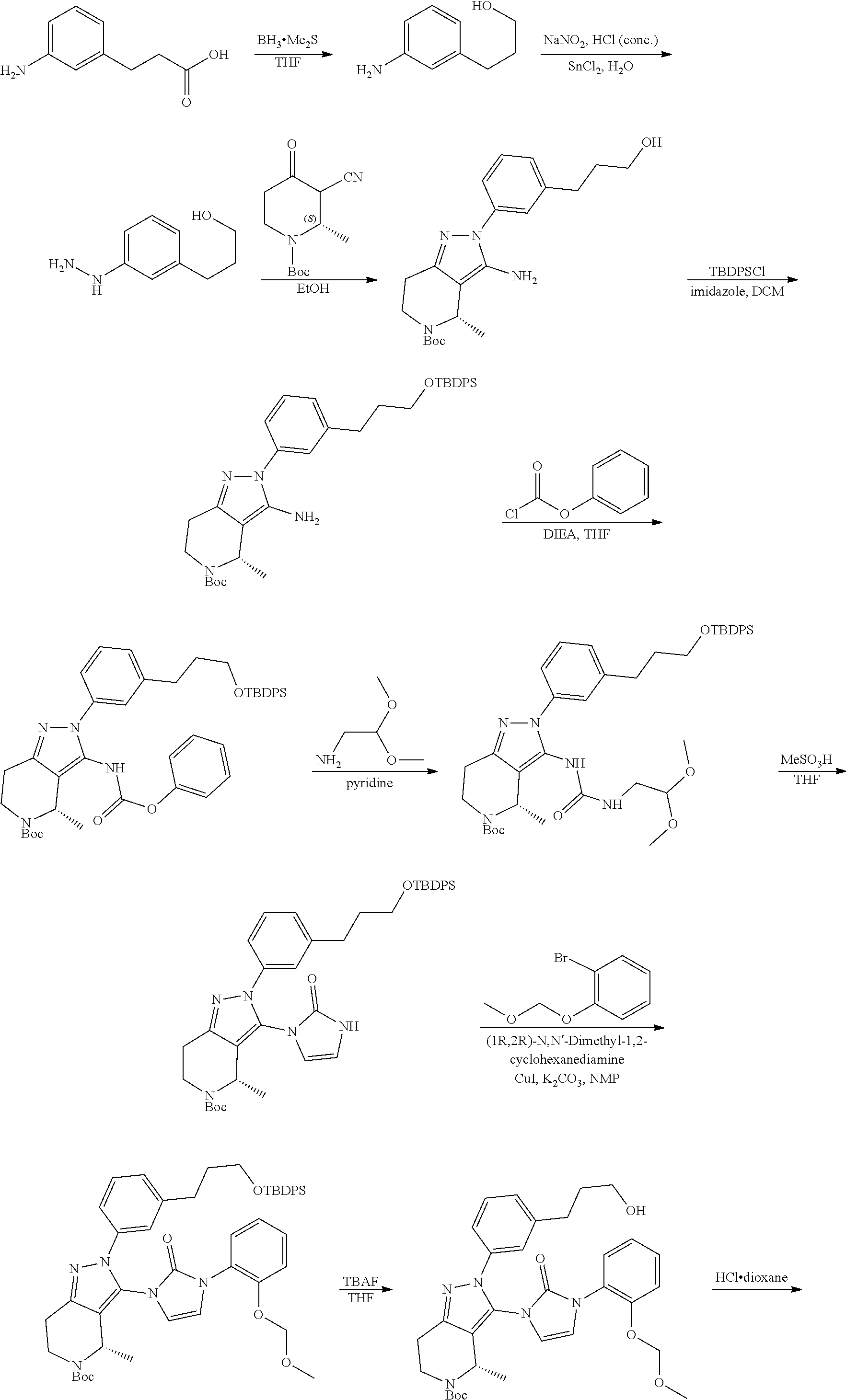

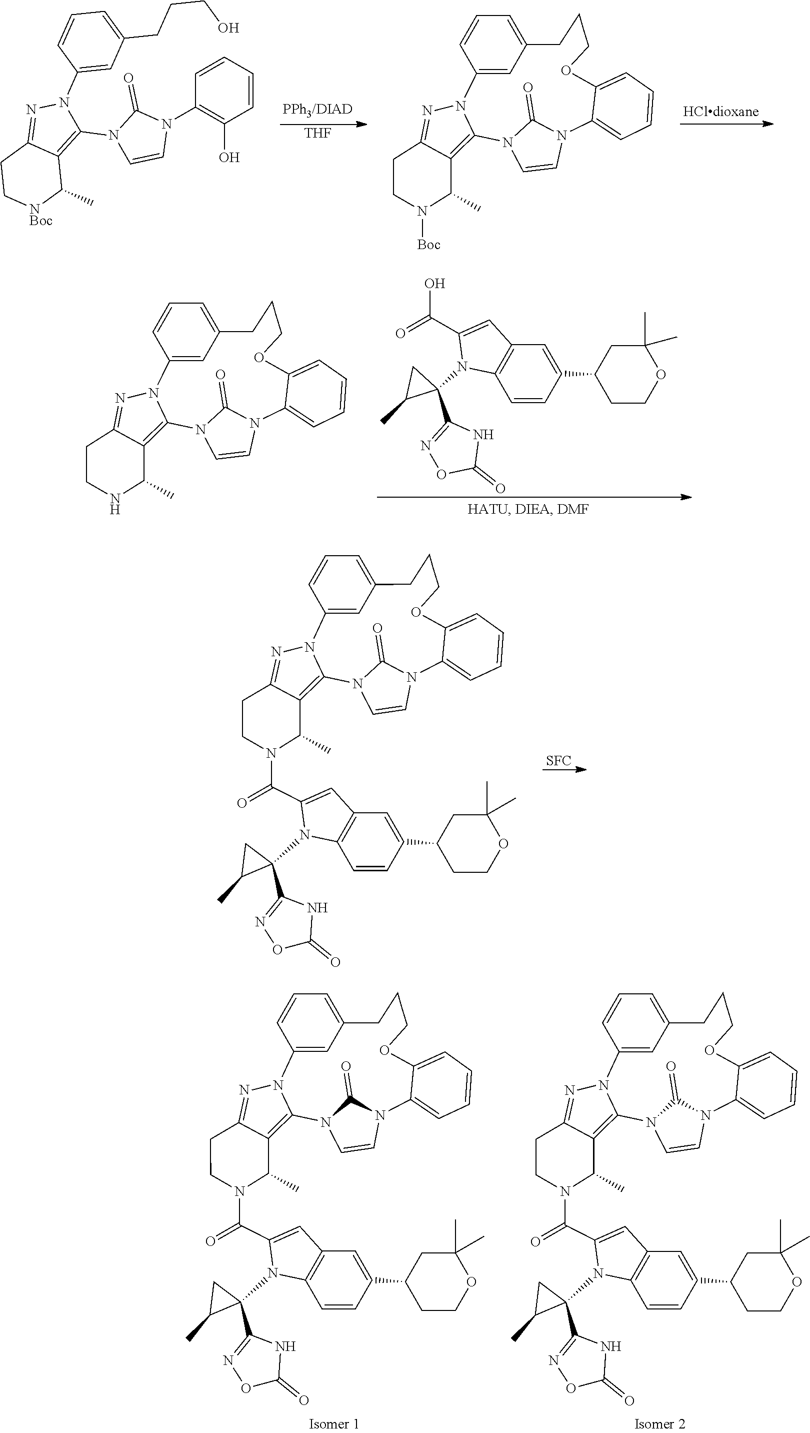

### Procedure for Synthesis of Compound 7

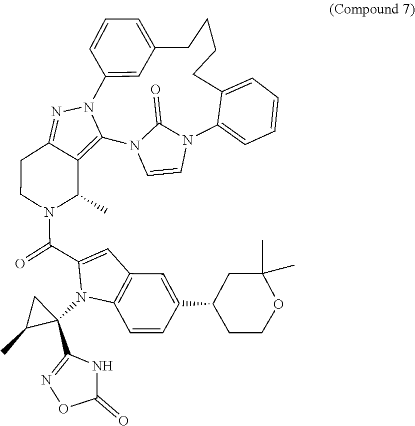

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-21-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a solution of 3-(3-aminophenyl) propionic acid (3 g, 18.2 mmol) in THF (30 mL) was added Borane-methyl sulfide complex (21.8 mL, 21.8 mmol) at 0° C. After addition, the mixture was allowed to warm to ambient temperature and stirred for 12 h. The reaction solution was quenched carefully by H₂O (100 mL) and extracted with EtOAc (2×100 mL). The combined organic layers were washed with brine (50 mL), then dried over Na₂SO₄, filtered and concentrated in vacuo. The crude product was purified by column chromatography (0-50% EtOAc in PE) to give 3-(3-aminophenyl)-1-propanol (2 g, 11.9 mmol, 73% yield) as colorless oil. m/z (ESI-pos) M+1=152.2

Step B: A solution of 3-(3-aminophenyl)-1-propanol (2 g, 11.9 mmol) in conc.HCl (4.5 mL) and H₂O (4.5 mL) at 0° C. was added NaNO₂ (904 mg, 13.1 mmol) in H₂O (6 mL) dropwise. After the mixture was stirred at 0° C. for 1 hour, SnCl₂ (4.97 g, 26.2 mmol) in conc. HCl (4.5 mL) and H₂O (4.5 mL) was added. The mixture was stirred at ambient temperature for another 1 hour. The reaction mixture was quenched with aqueous NaOH (10 N, 12 mL) to adjust pH to ~8-9 and the mixture was extracted with EtOAc (2×200 mL). The combined organics were washed with brine (50 mL), dried over Na₂SO₄, filtered and concentrated in vacuo. The crude product was purified by column chromatography (0-100% EtOAc in PE). to give 3-(3-hydrazineylphenyl) propan-1-ol (1.3 g, 7.04 mmol, 59% yield) as a yellow solid. m/z (ESI-pos) M+1=167.1

Step C: To a solution of 3-(3-hydrazineylphenyl)propan-1-ol (1.25 g, 6.77 mmol) in EtOH (20 mL) was added tert-butyl(2S)-3-cyano-2-methyl-4-oxo-1-piperidinecarboxylate (1.94 g, 8.12 mmol) and the mixture was stirred at 80° C. for 16 hours. The mixture was concentrated and the crude was purified by column chromatography (0-100 EtOAc in PE) to give tert-butyl (S)-3-amino-2-(3-(3-hydroxypropyl)phenyl)-4-methyl-2,4,6,7-tetrahydro-5H-pyrazolo[4,3-c]pyridine-5-carboxylate (1.3 g, 3.36 mmol, 45% yield) as a yellow solid. m/z (ESI-pos) M+1=387.2; ¹H NMR (400 MHz, CDCl₃) δ 7.40-7.32 (m, 3H), 7.18 (d, J=7.2 Hz, 1H), 5.21-5.03 (m, 1H), 4.41-4.24 (m, 1H), 3.72 (s, 1H), 3.65 (t, J=6.4 Hz, 2H), 3.12 (s, 1H), 2.78-2.74 (m, 2H), 2.69 (s, 2H), 1.93-1.86 (m, 2H), 1.50 (s, 9H), 1.35 (d, J=6.8 Hz, 3H).

Step D: To a solution of tert-butyl (S)-3-amino-2-(3-(3-hydroxypropyl)phenyl)-4-methyl-2,4,6,7-tetrahydro-5H-pyrazolo[4,3-c]pyridine-5-carboxylate (1.3 g, 3.03 mmol) in CH₂Cl₂ (26 mL) was added tert-butyl (chloro)bis(phenyl) silane (832 mg, 3.03 mmol) and imidazole (412 mg, 6.05 mmol). The mixture was stirred at 20° C. for 16 hours and then quenched with water (15 mL), extracted with CH₂Cl₂ (2×10 mL). The combined organics were dried over Na₂SO₄, filtered and concentrated under reduced pressure. The crude product was purified by column chromatography (0-25% EtOAc in PE) to give tert-butyl (S)-3-amino-2-(3-(3-((tert-butyldiphenylsilyl)oxy)propyl)phenyl)-4-methyl-2,4,6,7-tetrahydro-5H-pyrazolo[4,3-c]pyridine-5-carboxylate (1.5 g, 2.28 mmol, 75% yield) as a light yellow solid. m/z (ESI-pos) M+1=625.2; ¹HNMR (400 MHz, MeOD) δ 7.66-7.63 (m, 4H), 7.43-7.28 (m, 9H), 7.18 (d, J=7.6 Hz, 1H), 5.15 (q, J=6.4 Hz, 1H), 4.27 (s, 1H), 3.68 (t, J=6.4 Hz, 2H), 3.14 (s, 1H), 2.80 (t, J=7.2 Hz, 2H), 2.61-2.57 (m, 2H), 1.93-1.85 (m, 2H), 1.50 (s, 9H), 1.35 (d, J=6.4 Hz, 3H), 1.04 (s, 9H)

Step E: To a solution of tert-butyl (S)-3-amino-2-(3-(3-((tert-butyldiphenylsilyl)oxy)propyl)phenyl)-4-methyl-2,4,6,7-tetrahydro-5H-pyrazolo[4,3-c]pyridine-5-carboxylate (1.5 g, 2.28 mmol) in tetrahydrofuran (15 mL) at 0° C. was added N-ethyldiisopropylamine (884 mg, 6.84 mmol) and benzenyl chloroformate (714 mg, 4.56 mmol) and the mixture solution was stirred at 20° C. for 2 hours. Then the mixture was concentrated to provide crude tert-butyl (S)-2-(3-(3-((tert-butyldiphenyl silyl)oxy)propyl)phenyl)-4-methyl-3-((phenoxycarbonyl)amino)-2,4,6,7-tetrahydro-5H-pyrazolo[4,3-c]pyridine-5-carboxylate (1900 mg, 2.04 mmol) as a light yellow solid, which was used directly in the next step without further purification . . . m/z (ESI-pos) M+1=745.3

Step F: To a solution of tert-butyl (S)-2-(3-(3-((tert-butyldiphenyl silyl)oxy)propyl)phenyl)-4-methyl-3-((phenoxycarbonyl)amino)-2,4,6,7-tetrahydro-5H-pyrazolo[4,3-c]pyridine-5-carboxylate (1.9 g, 2.04 mmol) in pyridine (20 mL) was added 2,2-dimethoxyethylamine (644 mg, 6.12 mmol) and the mixture was stirred at 20° C. for 2 hours. The reaction mixture was concentrated to provide the crude product, which was purified by column chromatography (0-50% EtOAc in PE) to give tert-butyl (S)-2-(3-(3-((tert-butyldiphenylsilyl)oxy)propyl)phenyl)-3-(3-(2,2-dimethoxyethyl)ureido)-4-methyl-2,4,6,7-tetrahydro-5H-pyrazolo[4,3-c]pyridine-5-carboxylate (1.3 g, 1.63 mmol, 72% yield over 2 steps) as a white solid. m/z (ESI-pos) M+1=756.3

Step G: To a solution of tert-butyl (S)-2-(3-(3-((tert-butyldiphenylsilyl)oxy)propyl)phenyl)-3-(3-(2,2-dimethoxyethyl)ureido)-4-methyl-2,4,6,7-tetrahydro-5H-pyrazolo[4,3-c]pyridine-5-carboxylate (1.3 g, 1.63 mmol) in THF (20 mL) was added methanesulfonic acid (173 mg, 1.8 mmol). The mixture was stirred at 60° C. for 2 hr. Aqueous K₃PO₄ was added to adjust the pH to ~9, and then Boc₂O (71.1 mg, 0.33 mmol) was added, and the resulting mixture was stirred at 25° C. for 2 hrs. The reaction mixture was diluted with ethyl acetate (50 mL), washed with water (30 mL×2) and the aqueous layer was extracted with ethyl acetate (30 mL×2). The combined organic layers were washed with brine (50 mL) and dried over anhydrous Na₂SO₄. The mixture was filtered and concentrated under reduced pressure. The crude compound was purified by column chromatography (0-100% EtOAc in PE) to give tert-butyl (S)-2-(3-(3-((tert-butyldiphenylsilyl)oxy)propyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (1000 mg, 1.3 mmol) as a yellow solid. m/z (ESI-pos) M+1=692.3; ¹HNMR (400 MHz, DMSO) δ 10.33 (s, 1H), 7.62-7.60 (m, 4H), 7.48-7.40 (m, 6H), 7.31 (t, J=8.0 Hz, 1H), 7.21 (s, 1H), 7.15-7.13 (m, 2H), 6.51 (t, J=2.4 Hz, 1H), 6.48 (s, 1H), 5.07 (s, 1H), 4.20 (s, 1H), 3.64 (t, J=6.0 Hz, 2H), 3.11 (s, 1H), 2.74-2.63 (m, 4H), 1.83-1.76 (m, 2H), 1.43 (s, 9H), 1.13 (d, J=6.4 Hz, 3H), 1.01 (s, 9H).

Step H: To a solution of o-bromophenol (2 g, 11.6 mmol) in DCM (20 mL) at 0° C. was added DIPEA (3.14 g, 24.3 mmol) and bromomethoxymethane (1.73 g, 13.9 mmol). The mixture was allowed to warm to ambient temperature and stirred for 4 hours. Water (50 mL) was added and the mixture was extracted with DCM (2×100 mL), the combined organic layers were dried over anhydrous Na₂SO₄, filtered and concentrated under vacuum. The crude product was purified by column chromatography (0-10% EtOAc in PE) to give 1-bromo-2-(methoxymethoxy)benzene (2.2 g, 9.63 mmol) as a colorless oil. ¹H NMR (400 MHz, CDCl₃) δ 7.55-7.53 (m, 1H), 7.27-7.22 (m, 1H), 7.16-7.14 (m1H), 6.91-6.87 (m, 1H), 5.25 (s, 2H), 3.52 (s, 3H).

Step I: To a solution of(S)-2-(3-(3-((tert-butyldiphenylsilyl)oxy)propyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (0.9 g, 1.17 mmol) in NMP (34.1 mL) was added 1-bromo-2-(methoxymethoxy)benzene (508 mg, 2.34 mmol), Cuprous iodide (111 mg, 585 μmol), trans-(1R,2R) N,N′-Dimethyl-cyclohexane-1,2-diamine (167 mg, 1.17 mmol) and K₂CO₃ (485 mg, 3.51 mmol). The resulting mixture was stirred under N₂ at 130° C. for 3 hours. The reaction mixture was cooled to ambient temperature and diluted with water (80 mL), extracted with EtOAc (200 mL). The combined organics were washed with aqueous LiCl (3×50 mL), dried over anhydrous Na₂SO₄, filtered and the filtrate was concentrated under vacuum. The crude product was purified by column chromatography (0-25% EtOAc in PE) to give tert-butyl (4S)-2-(3-(3-((tert-butyldiphenylsilyl)oxy)propyl)phenyl)-3-(3-(2-(methoxymethoxy)phenyl)-2-oxo-2,3-dihydro-1H-imidazol-1-yl)-4-methyl-2,4,6,7-tetrahydro-5H-pyrazolo[4,3-c]pyridine-5-carboxylate (820 mg, 891 μmol) as a white solid. m/z (ESI-pos) M+1=828.3

Step J: To a solution of tert-butyl (4S)-2-(3-(3-((tert-butyldiphenylsilyl)oxy)propyl)phenyl)-3-(3-(2-(methoxymethoxy)phenyl)-2-oxo-2,3-dihydro-1H-imidazol-1-yl)-4-methyl-2,4,6,7-tetrahydro-5H-pyrazolo[4,3-c]pyridine-5-carboxylate (0.8 g, 869 μmol) in THF (30 mL) at 0° C. was added TBAF (1.0 M in THF, 1.3 mL, 1.3 mmol) and the mixture was allowed to warm to ambient temperature and stirred for 2 hours. Water (20 mL) was added and the mixture was extracted with EtOAc (3×20 mL). The organics were dried over anhydrous Na₂SO₄, filtered and concentrated to provide the crude product, which was purified by column chromatography (0-100% EtOAc in PE) to tert-butyl (4S)-2-(3-(3-hydroxypropyl)phenyl)-3-(3-(2-(methoxymethoxy)phenyl)-2-oxo-2,3-dihydro-1H-imidazol-1-yl)-4-methyl-2,4,6,7-tetrahydro-5H-pyrazolo[4,3-c]pyridine-5-carboxylate (520 mg, 794 μmol) as a white solid. m/z (ESI-pos) M+1=590.2

Step K: A solution of tert-butyl (4S)-2-(3-(3-hydroxypropyl)phenyl)-3-(3-(2-(methoxymethoxy)phenyl)-2-oxo-2,3-dihydro-1H-imidazol-1-yl)-4-methyl-2,4,6,7-tetrahydro-5H-pyrazolo[4,3-c]pyridine-5-carboxylate (520 mg, 794 μmol) in HCl/1,4-dioxane (4M, 10 mL) was stirred at 25° C. for 1 hour. Then directly concentrated under vacuum. The residue was diluted with DCM (15 mL) and aqueous K₃PO₄ was added to adjusted pH to ~9, then Boc₂O (207.7 mg, 0.95 mmol) was added. The resulting mixture was stirred at 25° C. for 2 hrs. Water (10 mL) was added and the mixture was extracted with EtOAc (3×100 mL), The combined organic layers were dried over anhydrous Na₂SO₄, filtered and concentrated. The crude product was purified by column chromatography (0-100% EtOAc in PE) to give tert-butyl (4S)-3-(3-(2-hydroxyphenyl)-2-oxo-2,3-dihydro-1H-imidazol-1-yl)-2-(3-(3-hydroxypropyl)phenyl)-4-methyl-2,4,6,7-tetrahydro-5H-pyrazolo[4,3-c]pyridine-5-carboxylate (330 mg, 544 μmol) as a white solid. m/z (ESI-pos) M+1=546.3; ¹H NMR (400 MHz, DMSO) δ 9.96 (s, 1H), 7.38-7.33 (m, 1H), 7.23-7.20 (m, 5H), 6.99 (d, J=7.2 Hz, 1H), 6.89-6.85 (m, 2H), 6.78 (s, 1H), 5.14 (s, 1H), 4.49 (s, 1H), 4.22 (s, 1H), 3.39 (s, 2H), 3.14 (s, 1H), 2.76-2.61 (m, 4H), 1.71-1.67 (m, 2H), 1.44 (s, 9H), 1.21 (d, J=6.0 Hz, 3H).

Step L: To a solution of tert-butyl tert-butyl (4S)-3-(3-(2-hydroxyphenyl)-2-oxo-2,3-dihydro-1H-imidazol-1-yl)-2-(3-(3-hydroxypropyl)phenyl)-4-methyl-2,4,6,7-tetrahydro-5H-pyrazolo[4,3-c]pyridine-5-carboxylate (290 mg, 478 μmol) in THF (29 mL) at 0° C. was added PPh₃ (314 mg, 1.2 mmol) and DIAD (242 mg, 1.2 mmol) under N₂ atmosphere. The mixture was stirred at 60° C. under N₂ for 2 hours. The reaction was cooled to ambient temperature and quenched with water (100 mL), extracted with EtOAc (3×100 mL). The combined organic layers were dried over anhydrous Na₂SO₄, filtered and concentrated. The crude product was purified by column chromatography (0-45% EtOAc in PE) to give tert-butyl (S)-8-methyl-30-oxo-21-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (185 mg, 316 μmol) as a white solid. m/z (ESI-pos) M+1=528.2

Step M: A solution of tert-butyl (S)-8-methyl-30-oxo-21-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (0.1 g, 171 μmol) in HCl/1,4-dioxane (4M, 2 mL) was stirred at ambient temperature for 1 hour. The mixture was concentrated to give (S)-8-methyl-21-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (80 mg, 168 μmol) as a white solid. m/z (ESI-pos) M+1=428.1

Step N: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (69.3 mg, 168 μmol) in DMF (1 mL) was added DIEA (87.1 mg, 674 μmol) and HATU (96.1 mg, 253 μmol). The mixture was stirred at ambient temperature for 30 mins, and then(S)-8-methyl-21-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (80 mg, 168 μmol) in DMF (0.5 mL) was added. The resulting mixture was stirred for 16 hours. Water (50 mL) was added and the reaction mixture was extracted with EtOAc (2×100 mL). The combined organic layers were washed with brine (50 mL), dried over Na₂SO₄, filtered and concentrated in vacuo. The crude product was purified by column chromatography (0-5% MeOH in DCM) to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-21-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (110 mg, 114 μmol) as a white solid. m/z (ESI-pos) M+1=821.3

Step O: (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-21-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (110 mg, 114 μmol) was dissolved in MeOH (150 mL) and separated by chiral supercritical fluid chromatography (separation condition: Daicel CHIRALPAK IA-H, 250 mm×30 mm I.D., 10 μm; Mobile Phase: CO₂/MeOH [0.2% NH₃ (7M Solution in MeOH)]=50/50 at 120 g/min; Temp: 35° C.; Wavelength: 214 nm) to afford the crude Isomer 1 (30 mg) and Isomer 2 (60 mg), which were further purified by prep-HPLC [Waters MS-triggered Prep-LC with SQD2 detector, Xbridge 5 u C18 150×19 mm, eluted with 80-100% ACN in H₂O (0.1% FA)] to give pure Isomer 1 (18.5 mg) and Isomer 2 (34.6 mg) as white solids (The absolute configuration was unknown, the configuration of two atropisomers were assigned arbitrarily).

Isomer 1: m/z (ESI-pos) M+1=821.3; ¹H NMR (400 MHz, DMSO) δ 11.59 (s, 1H), 7.50-7.45 (m, 2H), 7.42-7.38 (m, 4H), 7.24-7.22 (m, 1H), 7.20-7.17 (m, 2H), 7.11 (s, 1H), 7.08-7.05 (m, 1H), 6.87 (s, 1H), 6.65 (s, 1H), 6.59 (d, J=2.4 Hz, 1H), 5.59 (s, 1H), 4.42 (s, 1H), 3.91-3.87 (m, 2H), 3.73-3.70 (m, 2H), 3.59 (s, 1H), 3.02-2.98 (m, 2H), 2.89-2.80 (m, 2H), 2.75-2.68 (m, 1H), 2.15-2.11 (m, 1H), 2.04-1.98 (m, 2H), 1.70-1.67 (m, 3H), 1.62-1.55 (m, 2H), 1.53 (d, J=4.0 Hz, 3H), 1.27-1.25 (m, 6H), 1.19 (s, 4H).

Isomer 2: m/z (ESI-pos) M+1=821.3; ¹H NMR (400 MHz, DMSO) δ 11.66 (s, 1H), 7.51 (s, 1H), 7.47-7.45 (m, 1H), 7.42-7.39 (m3H), 7.37-7.34 (m, 1H), 7.24 (d, J=8.8 Hz, 1H), 7.18 (d, J=5.6 Hz, 3H), 7.08 (t, J=7.6 Hz, 1H), 6.84 (s, 1H), 6.68-6.59 (m, 2H), 5.60 (s, 1H), 4.42 (s, 1H), 3.94-3.89 (m, 1H), 3.83-3.78 (m, 1H), 3.73-3.71 (m, 2H), 3.58 (s, 1H), 3.03-2.99 (m, 2H), 2.90-2.79 (m, 2H), 2.76-2.70 (m, 1H), 2.17-1.98 (m, 3H), 1.74-1.68 (m, 3H), 1.65-1.56 (m, 2H), 1.51 (d, J=13.2 Hz, 3H), 1.28-1.25 (m, 4H), 1.19 (s, 6H).

### Example 8—Synthesis of Compound 8

### Synthetic Scheme

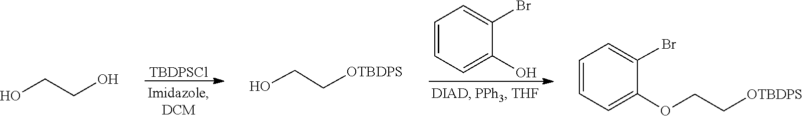

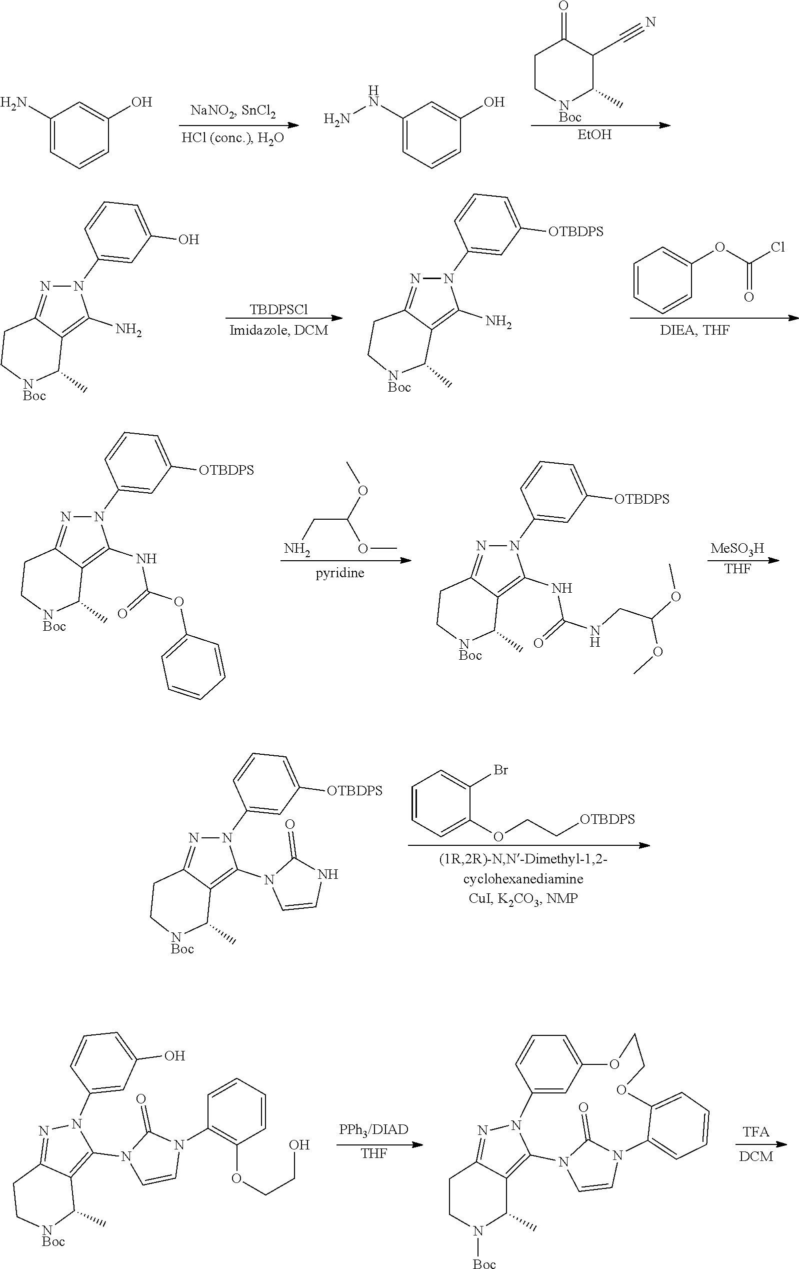

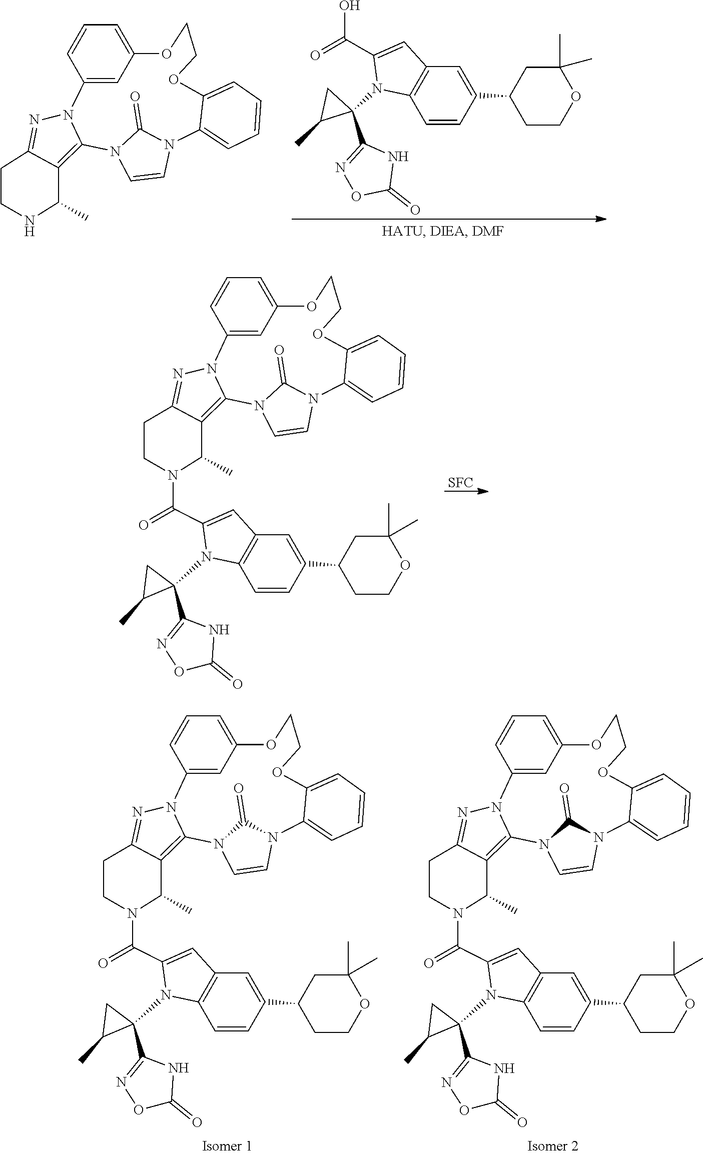

### Procedure for Synthesis of Compound 8

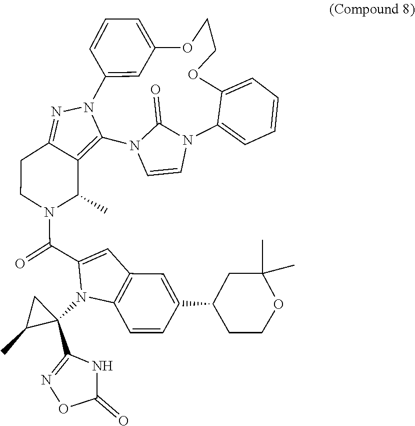

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-21,24-dioxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: A solution of 3-aminophenol (5 g, 45.8 mmol) in conc. HCl (50 mL) at 0° C. was treated with NaNO₂ (3.48 g, 50.4 mmol) in H₂O (25 mL) dropwise. After the mixture was stirred for 25 mins, SnCl₂ (19.1 g, 101 mmol) in conc. HCl (25 mL) was added dropwise and the mixture was stirred for 1 hour. The reaction was quenched with aqueous NaOH (10N, 90 mL) to adjust pH to about 9. Then the mixture was extracted with EtOAc (3×500 mL), the combined organic layers were dried over anhydrous Na₂SO₄, filtered and concentrated under vacuum. The crude product was purified by column chromatography (0-100% EtOAc in PE) to give 3-hydrazineylphenol (2.8 g, 20.3 mmol) as a yellow solid. m/z (ESI-pos) M+1=125.2; ¹H NMR (400 MHz, DMSO) δ 8.90 (s, 1H), 6.84 (t, J=7.6 Hz, 1H), 6.51 (s, 1H), 6.23 (t, J=2.0 Hz, 1H), 6.19-6.17 (m, 1H), 6.01-5.98 (m, 1H), 3.91 (s, 2H).

Step B: To a solution of 3-hydrazinophenol (1.4 g, 10.1 mmol) in EtOH (30 mL) was added tert-butyl(2S)-3-cyano-2-methyl-4-oxo-1-piperidinecarboxylate (2.42 g, 10.1 mmol). The mixture was stirred at 80° C. for 16 hours and then concentrated under reduced pressure to give the crude product, which was purified by column chromatography (0-100% EtOAc in PE) to give tert-butyl (S)-3-amino-2-(3-hydroxyphenyl)-4-methyl-2,4,6,7-tetrahydro-5H-pyrazolo[4,3-c]pyridine-5-carboxylate (2.6 g, 6.79 mmol) as a yellow solid. m/z (ESI-pos) M+1=345.1

Step C: To a solution of tert-butyl (S)-3-amino-2-(3-hydroxyphenyl)-4-methyl-2,4,6,7-tetrahydro-5H-pyrazolo[4,3-c]pyridine-5-carboxylate (1 g, 2.61 mmol) in CH₂Cl₂ (20 mL) at 0° C. was added imidazole (356 mg, 5.23 mmol) and tert-butyl (chloro)bis(phenyl) silane (790 mg, 2.87 mmol). The mixture was allowed to stir at ambient temperature for 2 hours. The reaction was quenched with H₂O (50 mL), extracted with CH₂Cl₂ (2×100 mL). The combined organic layers were dried over Na₂SO₄, filtered and concentrated under reduced pressure. The crude product was purified by column chromatography (0-25% EtOAc in PE) to give tert-butyl (S)-3-amino-2-(3-((tert-butyldiphenylsilyl)oxy)phenyl)-4-methyl-2,4,6,7-tetrahydro-5H-pyrazolo[4,3-c]pyridine-5-carboxylate (1.5 g, 2.32 mmol) as a yellow solid. m/z (ESI-pos) M+1=583.3

Step D: To a solution To a solution of tert-butyl (S)-3-amino-2-{m-[tert-butylbis(phenyl)siloxy]phenyl}-4-methyl-1,2,5-triaza-2,4,6,7-tetrahydro-5H-indene-5-carboxylate (1.5 g, 2.32 mmol) in THF (20 mL) at 0° C. were added DIEA (898 mg, 6.95 mmol) and benzenyl chloroformate (725 mg, 4.63 mmol). The mixture was allowed to stir at ambient temperature for 2 hours and then concentrated under vacuum to provide crude tert-butyl (S)-2-(3-((tert-butyldiphenylsilyl)oxy)phenyl)-4-methyl-3-((phenoxycarbonyl)amino)-2,4,6,7-tetrahydro-5H-pyrazolo[4,3-c]pyridine-5-carboxylate (2.7 g) as a light yellow solid, which was directly used in next step without further purification. m/z (ESI-pos) M+1=703.3

Step E: To a solution of tert-butyl (S)-2-(3-((tert-butyldiphenylsilyl)oxy)phenyl)-4-methyl-3-((phenoxycarbonyl)amino)-2,4,6,7-tetrahydro-5H-pyrazolo[4,3-c]pyridine-5-carboxylate (2.7 g, 2.3 mmol) in pyridine (20.8 mL) was added 2,2-dimethoxyethylamine (727 mg, 6.91 mmol) and the mixture was stirred at 20° C. for 2 hours. The reaction mixture was concentrated to provide the crude product, which was purified by column chromatography (0-50% EtOAc in PE) to give tert-butyl (S)-2-(3-((tert-butyldiphenylsilyl)oxy)phenyl)-3-(3-(2,2-dimethoxyethyl)ureido)-4-methyl-2,4,6,7-tetrahydro-5H-pyrazolo[4,3-c]pyridine-5-carboxylate (1.6 g, 2.02 mmol) as a white solid. m/z (ESI-pos) M+1=714.3

Step F: To a solution of tert-butyl (S)-2-(3-((tert-butyldiphenylsilyl)oxy)phenyl)-3-(3-(2,2-dimethoxyethyl)ureido)-4-methyl-2,4,6,7-tetrahydro-5H-pyrazolo[4,3-c]pyridine-5-carboxylate (1.6 g, 2.02 mmol) in THF (30 mL) was added methanesulfonic acid (194 mg, 2.02 mmol) and the mixture was stirred at 60° C. for 1 hour. The reaction was quenched by the addition of H₂O (50 mL) and the mixture was extracted with EtOAc (2×100 mL). The combined organic layers were dried over anhydrous Na₂SO₄, filtered and concentrated. The crude product was purified by column chromatography (0-100% EtOAc in PE) to give tert-butyl (4S)-2-(3-((tert-butyldiphenylsilyl)oxy)phenyl)-4-methyl-3-(2-oxo-2,3-dihydro-1H-imidazol-1-yl)-2,4,6,7-tetrahydro-5H-pyrazolo[4,3-c]pyridine-5-carboxylate (1.2 g, 1.66 mmol) as a white solid. m/z (ESI-pos) M+1=650.3

Step G: To a solution of ethylene glycol (2 g, 32.2 mmol) in CH₂Cl₂ (20 mL) was added tert-butyl (chloro)bis(phenyl) silane (8.86 g, 32.2 mmol) and imidazole (4.39 g, 64.4 mmol). The mixture was stirred at ambient temperature for 16 hours. The reaction was quenched with water (50 mL), extracted with CH₂Cl₂ (2×100 mL). The combined organic layers were dried over Na₂SO₄, filtered and concentrated under reduced pressure. The crude product was purified by column chromatography (0-25% EtOAc in PE) to give 2-[tert-butylbis(phenyl)siloxy]ethanol (4 g, 12 mmol) as colorless oil. m/z (ESI-pos) M+1=323.1; ¹H NMR (400 MHz, CDCl₃) δ 7.68-7.66 (m, 4H), 7.46-7.37 (m, 6H), 3.78-3.75 (m, 2H), 3.69-3.67 (m, 2H), 2.15-2.11 (m, 1H), 1.08-1.07 (m, 9H).

Step H: To a solution of 2-[tert-butylbis(phenyl)siloxy]ethanol (2 g, 5.99 mmol) in THF (20 mL) was added o-bromophenol (1.04 g, 5.99 mmol) and triphenylphosphine (3.14 g, 12 mmol) at 0° C. under N₂ atmosphere. Then isopropyl (oxycarbonylamino) 2-propanecarbamate (2.42 g, 12 mmol) was added dropwise. The resulting mixture was allowed to warm to ambient temperature and stir for 2 hours. The reaction was quenched with water (50 mL), extracted with CH₂Cl₂ (2×100 mL). The combined organics were dried over Na₂SO₄, filtered and concentrated under vacuum. The crude product was purified by column chromatography (0-25% EtOAc in PE) to give (2-(2-bromophenoxy) ethoxy)(tert-butyl)diphenylsilane (1.2 g, 2.37 mmol) as colorless oil. ¹H NMR (400 MHz, CDCl₃) δ 7.74-7.72 (m, 4H), 7.55-7.53 (m, 1H), 7.43-7.36 (m, 6H), 7.24-7.20 (m, 1H), 6.89-6.80 (m, 2H), 4.16 (t, J=5.2 Hz, 2H), 4.03 (t, J=5.2 Hz, 2H), 1.07-1.05 (m, 9H).

Step I: To a solution of tert-butyl (4S)-2-(3-((tert-butyldiphenylsilyl)oxy)phenyl)-4-methyl-3-(2-oxo-2,3-dihydro-1H-imidazol-1-yl)-2,4,6,7-tetrahydro-5H-pyrazolo[4,3-c]pyridine-5-carboxylate (1.1 g, 1.52 mmol) in NMP (20 mL) was added (2-(2-bromophenoxy) ethoxy)(tert-butyl)diphenylsilane (1.04 g, 2.29 mmol), copper iodide (145 mg, 762 μmol), (1S,2S)-1,2-bis(methylamino)cyclohexane (217 mg, 1.52 mmol) and K₂CO₃ (632 mg, 4.57 mmol). The mixture was stirred at 130° C. for 3 hours. The reaction was cooled to ambient temperature and quenched with water (100 mL), extracted with EtOAc (2×100 mL). The combined organic layers were washed with aqueous LiCl (2×50 mL), dried over Na₂SO₄, filtered and concentrated in vacuo. The crude product was purified by column chromatography (0-100% EtOAc in PE) to give tert-butyl (4S)-3-(3-(2-(2-hydroxyethoxy)phenyl)-2-oxo-2,3-dihydro-1H-imidazol-1-yl)-2-(3-hydroxyphenyl)-4-methyl-2,4,6,7-tetrahydro-5H-pyrazolo[4,3-c]pyridine-5-carboxylate (0.5 g, 822 μmol) as a white solid. m/z (ESI-pos) M+1=548.3

Step J: To a solution of tert-butyl (4S)-3-(3-(2-(2-hydroxyethoxy)phenyl)-2-oxo-2,3-dihydro-1H-imidazol-1-yl)-2-(3-hydroxyphenyl)-4-methyl-2,4,6,7-tetrahydro-5H-pyrazolo[4,3-c]pyridine-5-carboxylate (0.5 g, 822 μmol) in THF (28 mL) was added triphenylphosphine (539 mg, 2.05 mmol) and isopropyl (oxycarbonylamino) 2-propanecarbamate (415 mg, 2.05 mmol) dropwise at 0° C. under N₂ atmosphere. The mixture was warmed 60° C. where it stirred for 2 hours. The reaction mixture was concentrated under reduced pressure and the residue was purified by column chromatography (0-75% EtOAc in PE) to give tert-butyl (S)-8-methyl-30-oxo-21,24-dioxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (0.7 g, 661 μmol) as a white solid. m/z (ESI-pos) M+1=530.2

Step K: To a solution of tert-butyl (S)-8-methyl-30-oxo-21,24-dioxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (0.3 g, 283 μmol) in CH₂Cl₂ (4 mL) under N₂ atmosphere at 0° C. and was added trifluoroacetic acid (2 mL) and the mixture was stirred for 1 hour. The mixture was concentrated to give crude(S)-8-methyl-21,24-dioxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one trifluoroacetic acid (260 mg, 285 μmol) as a white solid. m/z (ESI-pos) M+1=430.2

Step L: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (0.1 g, 243 μmol) in DMF (2.5 mL) was added DIEA (126 mg, 972 μmol) and HATU (139 mg, 365 μmol). The mixture was stirred at 20° C. for 30 mins, and then(S)-8-methyl-21,24-dioxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one trifluoroacetic acid (244 mg, 267 μmol) was added. The resulting mixture was stirred at 20° C. for 16 hours. The reaction was quenched with water (50 mL), extracted with EtOAc (2×100 mL). The combined organics were washed with brine (2×50 mL), dried over Na₂SO₄, filtered and concentrated to provide the crude product. The crude product was purified by column chromatography (0-5% MeOH in CH₂Cl₂) to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-21,24-dioxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (0.3 g, 182 μmol) as a white solid. m/z (ESI-pos) M+1=823.3

Step M: (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-21,24-dioxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (0.3 g, 182 μmol) was dissolved in MeOH (150 mL) and separated by chiral supercritical fluid chromatography (separation condition: Daicel CHIRALCEL IH, 250 mm×30 mm I.D., 10 μm; Mobile Phase: CO2/MeOH [0.2% NH3 (7M Solution in MeOH)]=50/50 at 80 g/min; Temp: 35° C.; Wavelength: 214 nm) to afford the crude Isomer 1 (60 mg) and Isomer 2 (50 mg), which was further purified by prep-HPLC [Waters MS-triggered Prep-LC with SQD2 detector, Xbridge 5 u C18 150×19 mm, eluted with 70-100% ACN in H₂O (0.1% FA)] to give pure Isomer 1 (31.35 mg) and Isomer 2 (25.28 mg) as white solids (The absolute configuration was unknown, the configuration of two atropisomers were assigned arbitrarily).

Isomer 1: m/z (ESI-pos) M+1=823.3; ¹H NMR (400 MHz, DMSO) δ 11.62 (s, 1H), 7.51 (s, 1H), 7.44-7.34 (m, 4H), 7.24 (d, J=8.4 Hz, 2H), 7.18-7.14 (m, 2H), 7.05 (t, J=7.2 Hz, 1H), 6.93-6.91 (m, 1H), 6.84 (s, 1H), 6.57-6.48 (m, 2H), 5.58 (s, 1H), 4.56-4.50 (m, 1H), 4.39-4.31 (m, 3H), 4.21 (t, J=9.2 Hz, 1H), 3.73-3.71 (m, 2H), 3.59 (s, 1H), 3.03-2.99 (m, 2H), 2.90-2.87 (m, 1H), 1.74-1.68 (m, 4H), 1.65-1.56 (m, 2H), 1.51 (d, J=12.8 Hz, 3H), 1.28-1.25 (m, 4H), 1.19 (s, 6H).

Isomer 2: m/z (ESI-pos) M+1=823.3; ¹H NMR (400 MHz, DMSO) δ 11.58 (s, 1H), 7.49 (s, 1H), 7.45-7.36 (m, 4H), 7.23-7.12 (m, 4H), 7.05 (t, J=7.2 Hz, 1H), 6.96-6.93 (m, 1H), 6.87 (s, 1H), 6.56 (s, 1H), 6.42 (d, J=2.8 Hz, 1H), 5.59 (s, 1H), 4.53-4.47 (m, 1H), 4.39-4.32 (m, 3H), 4.23-4.18 (m, 1H), 3.72-3.70 (m, 2H), 3.57 (s, 1H), 3.02-2.97 (m, 2H), 2.89-2.85 (m, 1H), 1.71-1.67 (m, 4H), 1.61-1.55 (m, 2H), 1.50 (d, J=12.0 Hz, 3H), 1.27-1.25 (m, 4H), 1.18 (s, 6H).

### Example 9—Synthesis of Compound 9

### Synthetic Scheme

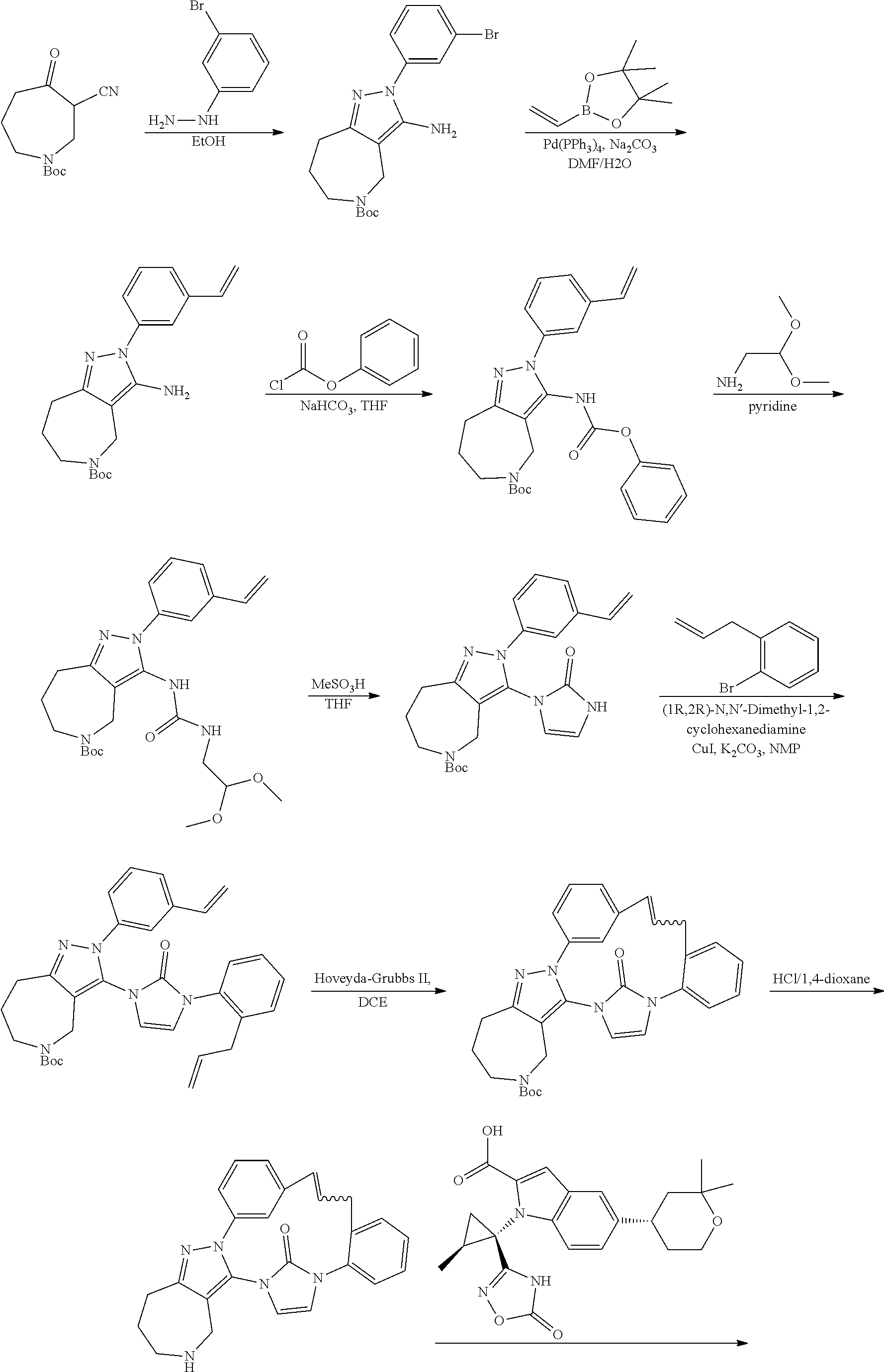

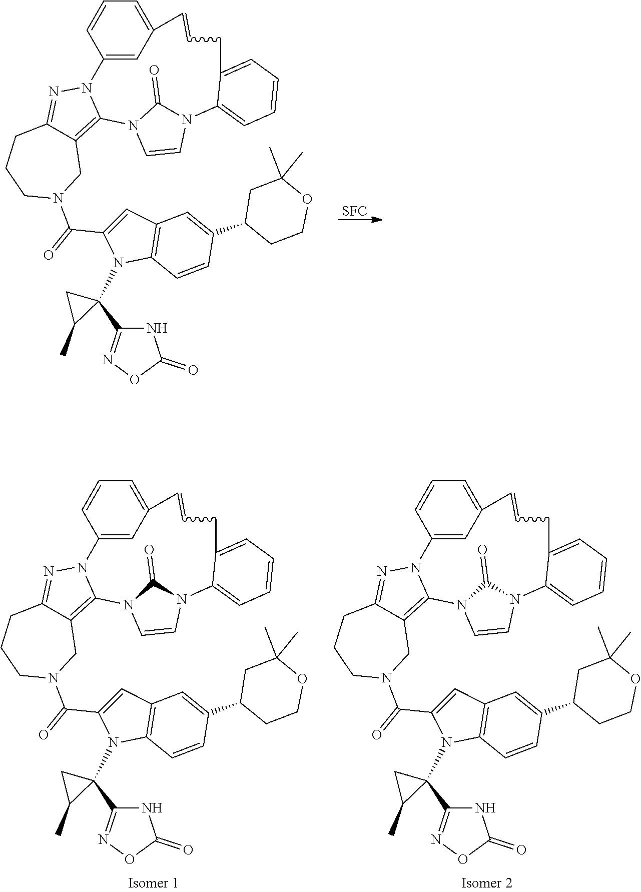

### Procedure for Synthesis of Compound 9

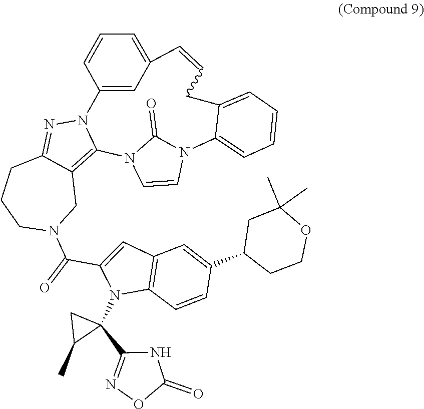 [SMILES: C[C@H]1C[C@]1(c1noc(=O)[nH]1)n1c(C(=O)N2CCCc3nn4c(c3C2)-n2ccn(c2=O)-c2ccccc2CC=Cc2cccc-4c2)cc2cc([C@H]3CCOC(C)(C)C3)ccc21]

### 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,23,25,27-decaen-30-one

Step A: To a solution of tert-butyl 3-cyano-4-oxoazepane-1-carboxylate (2.8 g, 11.76 mmol) in Ethanol (110 mL) was added (3-bromophenyl)hydrazine (2.41 g, 12.94 mmol) at 25° C. The resulting mixture was warmed to 80° C. where it stirred for 8 hr. The reaction mixture was cooled to room temperature, diluted with ethyl acetate (50 mL) and washed with water (50 mL×3). The aqueous layer was extracted with ethyl acetate (40 mL×2). The combined organic layers were washed with brine (50 mL), dried over anhydrous Na₂SO₄, filtered and concentrated under reduced pressure to give the crude compound. The crude compound was purified by column chromatography (0-60% EtOAc in PE) to give tert-butyl 3-amino-2-(3-bromophenyl)-2,6,7,8-tetrahydropyrazolo[4,3-c]azepine-5(4H)-carboxylate (4.0 g, 8.47 mmol) as a yellow solid. m/z (ESI-pos) M+1=407.1/409.1

Step B: To a mixture of tert-butyl 3-amino-2-(3-bromophenyl)-2,6,7,8-tetrahydro pyrazolo[4,3-c]azepine-5(4H)-carboxylate (1.8 g, 4.43 mmol), 4,4,5,5-tetramethyl-2-vinyl-1,3,2-dioxaborolane (3.41 g, 22.16 mmol) and Na₂CO₃ (939 mg, 8.86 mmol) in DMF/H₂O (30 mL/6 mL) was added Pd(PPh₃)₄ (511 mg, 0.443 mmol) at 25° C. under N₂ protection. The resulting mixture was stirred at 80° C. for 16 hr under N₂ and then cooled down to room temperature, and diluted with ethyl acetate (50 mL) and the organic phase was washed with water (50 mL×3). The aqueous layer was extracted with ethyl acetate (30 mL×3). The combined organic layers were washed with brine (50 mL×3), dried over anhydrous Na₂SO₄, filtered and concentrated under reduced pressure. The crude residue was purified by column chromatography (0-60% EtOAc in PE) to give tert-butyl 3-amino-2-(3-vinylphenyl)-2,6,7,8-tetrahydropyrazolo[4,3-c]azepine-5(4H)-carboxylate (1.5 g, 2.92 mmol) as a yellow oil. m/z (ESI-pos) M+1=355.2

Step C: To a solution of tert-butyl 3-amino-2-(3-vinylphenyl)-2,6,7,8-tetrahydropyrazolo[4,3-c]azepine-5(4H)-carboxylate (1.5 g, 4.23 mmol), NaHCO₃ (1.06 g, 12.69 mmol) in Tetrahydrofuran (15 mL) was added benzenyl chloroformate (1.31 g, 8.46 mmol) at 0° C. The reaction mixture was warmed to 30° C. where it stirred for 2 hr. The reaction mixture was directly concentrated to give crude tert-butyl 3-((phenoxycarbonyl)amino)-2-(3-vinylphenyl)-2,6,7,8-tetrahydropyrazolo[4,3-c]azepine-5(4H)-carboxylate (1.6 g crude) as a yellow oil, which was used directly for next step without any further purification. m/z (ESI-pos) M+1=475.1

Step D: Crude tert-butyl 3-((phenoxycarbonyl)amino)-2-(3-vinylphenyl)-2,6,7,8-tetrahydropyrazolo[4,3-c]azepine-5(4H)-carboxylate (1.6 g, 4.23 mmol) was dissolved in pyridine (15 mL), to which 2,2-dimethoxyethylamine (888 mg, 8.46 mmol) was added. The mixture was stirred at 25° C. for 3 hr and then concentrated under reduced pressure to give the crude product, which was purified by column chromatography (0-60% EtOAc in PE) to give tert-butyl (S)-4-methyl-3-(2-oxo-2,3-dihydro-1H-imidazol-1-yl)-2-(3-vinylphenyl)-2,4,6,7-tetrahydro-5H-pyrazolo[4,3-c]pyridine-5-carboxylate (1.4 g, 2.59 mmol) as a yellow solid. m/z (ESI-pos) M+1=486.3

Step E: To a solution of tert-butyl 3-(2-oxo-2,3-dihydro-1H-imidazol-1-yl)-2-(3-vinylphenyl)-2,6,7,8-tetrahydropyrazolo[4,3-c]azepine-5(4H)-carboxylate (1.4 g, 2.88 mmol) in THF (20 mL) was added methanesulfonic acid (277 mg, 2.88 mmol). The mixture was stirred at 60° C. for 2 hr. Aqueous K₃PO₄ was then added to the reaction mixture to adjust pH to ~9, and then Boc₂O (627 mg, 2.88 mmol) was added, and the resulting mixture was stirred at 25° C. for 16 hr. The reaction mixture was diluted with ethyl acetate (50 mL), washed with water (30 mL×3) and the aqueous layer was extracted with ethyl acetate (30 mL×3). The combined organic layers were washed with brine (50 mL) and dried over anhydrous Na₂SO₄, filtered and evaporated under reduced pressure to give the crude compound. The crude compound was purified by column chromatography (0-100% EtOAc in PE) to give tert-butyl 3-(2-oxo-2,3-dihydro-1H-imidazol-1-yl)-2-(3-vinylphenyl)-2,6,7,8-tetrahydropyrazolo[4,3-c]azepine-5 (4H)-carboxylate (1.1 g, 2.35 mmol) as a yellow solid. m/z (ESI-pos) M+1=422.2

Step F: To a solution of tert-butyl 3-(2-oxo-2,3-dihydro-1H-imidazol-1-yl)-2-(3-vinylphenyl)-2,6,7,8-tetrahydropyrazolo[4,3-c]azepine-5(4H)-carboxylate (1100 mg, 2.61 mmol) and 1-allyl-2-bromobenzene (668 mg, 3.39 mmol) in NMP (10 mL) were added (1R,2R)-1,2-bis(methylamino)cyclohexane (185 mg, 1.30 mmol), CuI (497 mg, 2.61 mmol) and K₂CO₃ (1.08 g, 7.83 mmol). The reaction mixture was heated at 130° C. for 3 hr under N₂. The reaction mixture was cooled to ambient temperature and diluted with ethyl acetate (50 mL), washed with water (30 mL×3) and the aqueous layer was extracted with ethyl acetate (30 mL×3). The combined organic layers were washed with brine (50 mL), dried over anhydrous Na₂SO₄, filtered and concentrated under reduced pressure. The crude compound was purified by column chromatography (0-100% EtOAc in PE) to give tert-butyl 3-(3-(2-allylphenyl)-2-oxo-2,3-dihydro-1H-imidazol-1-yl)-2-(3-vinylphenyl)-2,6,7,8-tetrahydropyrazolo[4,3-c]azepine-5(4H)-carboxylate (675 mg) as a yellow solid. m/z (ESI-pos) M+1=538.3

Step G: To a solution of tert-butyl 3-(3-(2-allylphenyl)-2-oxo-2,3-dihydro-1H-imidazol-1-yl)-2-(3-vinylphenyl)-2,6,7,8-tetrahydropyrazolo[4,3-c]azepine-5(4H)-carboxylate (370 mg, 0.65 mmol) in DCE (350 mL) was added Hoveyda-Grubbs II (370 mg, 0.59 mmol) at 25° C. under N₂ protection. The reaction mixture was heated to 80° C. where it stirred for 3 hr under N₂. The reaction mixture was cooled to ambient temperature and quenched with water (300 mL) and the aqueous layer was extracted with dichloromethane (50 mL×3). The combined organic layers were washed with brine (50 mL), dried over anhydrous Na₂SO₄, filtered and concentrated under reduced pressure to give the crude compound, which was purified by column chromatography (0-100% EtOAc in PE) to give tert-butyl 30-oxo-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,23,25,27-decaene-8-carboxylate (220 mg) as a yellow solid. m/z (ESI-pos) M+1=510.3

Step H: tert-butyl 30-oxo-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,23,25,27-decaene-8-carboxylate (150 mg, 0.294 mmol) was dissolved in HCl/1,4-dioxane (4M, 6 mL) and stirred at 25° C. for 1 h. The reaction mixture was directly concentrated under reduced pressure to give crude 2,3,8,12,15-pentaazahexacyclo[23.3.1.1¹²,¹⁵.0²,¹¹.0⁴,¹⁰.0¹⁶,²¹]triaconta-1(29),3,10,13,16,18,20,23,25,27-decaen-30-one hydrogen chloride (170 mg, 0.29 mmol) as a yellow solid. m/z (ESI-pos) M+1=410.2 Step I: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (121 mg, 0.294 mmol) in DMF (5 mL) was added DIEA (114 mg, 0.882 mmol) and HATU (168 mg, 0.441 mmol) at 25° C. The mixture stirred at ambient temperature for 10 min, then 2,3,8,12,15-pentaazahexacyclo[23.3.1.1¹²,¹⁵.0²,¹¹.0⁴,¹⁰.0¹⁶,²¹]triaconta-1(29),3,10,13,16,18,20,23,25,27-decaen-30-one hydrogen chloride (150 mg crude, 0.294 mmol) was added to the mixture. The mixture was stirred at ambient temperature overnight. The reaction mixture was diluted with ethyl acetate (30 mL), washed with water (30 mL×2). The aqueous layer was extracted with ethyl acetate (30 mL×2). The combined organic layers were washed with brine (30 mL), dried over anhydrous Na₂SO₄, filtered and concentrated under reduced pressure. The crude compound and purified by prep-HPLC [Waters MS-triggered Prep-LC with QDA detector, WELCH Xtimate C18 21.2*250 mm 10 μm, eluted with 60-100% ACN in H₂O (0.1% FA)] to give 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,23,25,27-decaen-30-one (90 mg) as a yellow solid. m/z (ESI-pos) M+1=803.3

Step J: 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,23,25,27-decaen-30-one (90 mg, 0.11 mmol) was dissolved in MeOH (150 mL) and separated by chiral supercritical fluid chromatography (separation condition: Daicel CHIRALPAK OD_3, 3*150 mm, 3 um; Mobile Phase: CO2/MeOH (0.1% DEA)=50/50 at 1.5 mL/min; Temp: 37° C.; Wavelength: 254 nm) to afford Isomer 1 (40.3 mg) and Isomer 2 (25.1 mg) as white solids (The absolute configuration was unknown, the configuration of two atropisomers were assigned arbitrarily).

Isomer 1: m/z (ESI-pos) M+1=803.3; ¹H NMR (400 MHz, DMSO) δ 9.04 (s, 1H), 7.62-7.55 (m, 2H), 7.48-7.28 (m, 6H), 7.15 (d, J=8.4 Hz, 1H), 7.10 (d, J=7.6 Hz, 1H), 6.80 (s, 1H), 6.61-6.53 (m, 4H), 5.90-5.84 (m, 1H), 4.77 (s, 1H), 4.06-3.89 (m, 2H), 3.73-3.68 (m, 2H), 3.27-3.14 (m, 4H), 2.89-2.84 (m, 2H), 2.02-1.93 (m, 2H), 1.74-1.67 (m, 3H), 1.58-1.48 (m, 4H), 1.26 (s, 3H), 1.15-1.08 (m, 6H).

Isomer 2: m/z (ESI-pos) M+1=803.3; ¹H NMR (400 MHz, DMSO) δ 11.35 (s, 1H), 7.62-7.58 (m, 2H), 7.50-7.32 (m, 6H), 7.21 (d, J=8.4 Hz, 1H), 7.11 (d, J=7.6 Hz, 1H), 6.79 (s, 2H), 6.63-6.42 (m, 3H), 5.90-5.84 (m, 1H), 5.06-4.65 (m, 2H), 3.71 (d, J=8.0 Hz, 2H), 3.32-3.15 (m, 2H), 2.18-1.99 (m, 3H), 1.65-1.43 (m, 8H), 1.30-1.22 (m, 6H), 1.14 (s, 6H).

### Example 10—Synthesis of Compound 10

### Synthetic Scheme

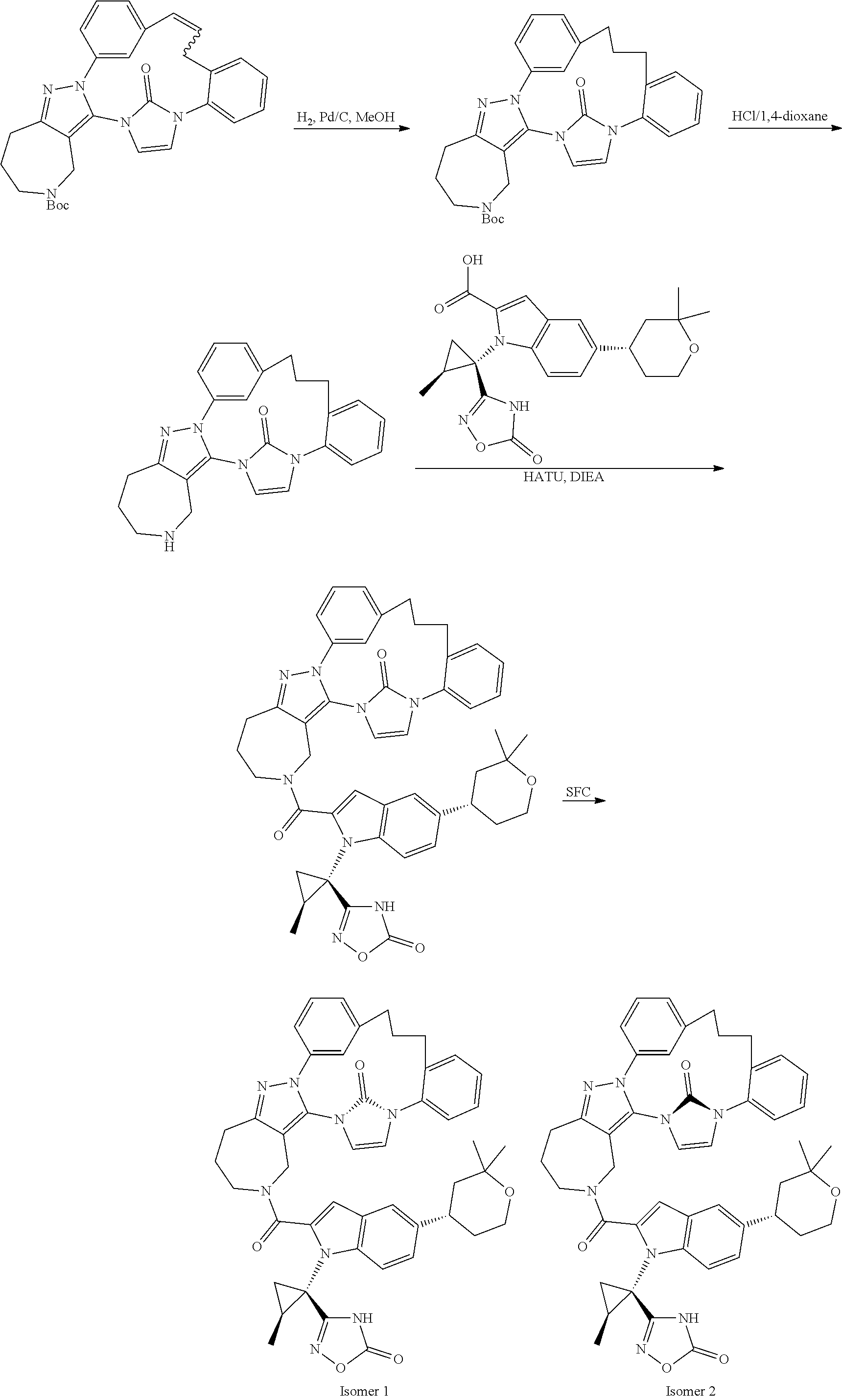

### Procedure for Synthesis of Compound 10

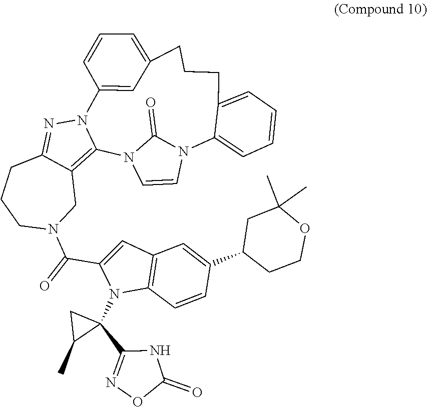 [SMILES: C[C@H]1C[C@]1(c1noc(=O)[nH]1)n1c(C(=O)N2CCCc3nn4c(c3C2)-n2ccn(c2=O)-c2ccccc2CCCc2cccc-4c2)cc2cc([C@H]3CCOC(C)(C)C3)ccc21]

### 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaen-30-one

Step A: To a solution of tert-butyl 30-oxo-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,23,25,27-decaene-8-carboxylate (210 mg, 0.41 mmol) in methanol (10 mL) was added Pd/C (105 mg, 50% w/w) at 25° C. The mixture was evacuated and backfilled with H₂ and then stirred under H₂ atmosphere at 25° C. for 16 hr. The reaction mixture was filtered through a pad of Celite and the filtrate was evaporated under reduced pressure to give tert-butyl 30-oxo-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaene-8-carboxylate (120 mg, 0.199 mmol) as a yellow solid. m/z (ESI-pos) M+1=512.2 Step B: tert-butyl 30-oxo-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaene-8-carboxylate (120 mg, 0.23 mmol) was dissolved in HCl/1,4-dioxane (4M, 6 mL) and stirred at 25° C. for 1 h. The reaction mixture was concentrated under reduced pressure to give crude 2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaen-30-one hydrogen chloride (130 mg, 0.23 mmol) as a yellow solid. m/z (ESI-pos) M+1=412.3

Step C: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (94 mg, 0.23 mmol) in DMF (5 mL) was added DIEA (89 mg, 0.69 mmol) and HATU (131 mg, 0.345 mmol) at 25° C., the mixture stirred for 10 min, then 2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaen-30-one hydrogen chloride (130 mg crude, 0.23 mmol) was added to the mixture. The mixture was stirred at ambient temperature overnight. The reaction mixture was diluted with ethyl acetate (30 mL), washed with water (30 mL×2). The aqueous layer was extracted with ethyl acetate (30 mL×2). The combined organic layers were washed with brine (30 mL), dried over anhydrous Na₂SO₄, filtered and concentrated under reduced pressure to give the crude compound which was purified by prep-HPLC [Instrument: Waters MS-triggered Prep-LC with QDA detector; Column: WELCH Xtimate C18 21.2*250 mm 10 μm; Gradient: 69-79% ACN in H₂O (0.1% FA) in 10 min; Flow rate: 30 mL/min] to give 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaen-30-one (60 mg) as a yellow solid. m/z (ESI-pos) M+1=805.3

Step D: 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaen-30-one (60 mg, 0.07 mmol) was dissolved in MeOH (150 mL) and separated by chiral supercritical fluid chromatography [separation condition: Daicel CHIRALPAK OX_3, 3*150 mm, 3 um; Mobile Phase: CO2/MeOH (0.1% DEA)=50/50 at 1.5 mL/min; Temp: 37° C.; Wavelength: 254 nm] to afford Isomer 1(24.82 mg) and Isomer 2 (19.15 mg) as white solids (The absolute configuration was unknown, the configuration of two atropisomers were assigned arbitrarily).

Isomer 1: m/z (ESI-pos) M+1=805.3; ¹H NMR (400 MHz, DMSO) δ 11.34 (s, 1H), 7.44-7.26 (m, 8H), 7.18-7.15 (m, 2H), 6.92 (s, 1H), 6.70 (s, 1H), 6.51-6.19 (m, 2H), 4.96-4.68 (m, 2H), 3.74-3.68 (m, 2H), 2.86-2.65 (m, 4H), 2.41-2.31 (m, 1H), 2.06-1.97 (m, 4H), 1.85-1.79 (m, 3H), 1.64-1.43 (m, 6H), 1.24 (s, 3H), 1.17-1.12 (m, 8H).

Isomer 2: m/z (ESI-pos) M+1=805.3; ¹H NMR (400 MHz, DMSO) δ 11.21 (s, 1H), 7.44-7.32 (m, 7H), 7.18-7.14 (m, 3H), 6.90 (s, 1H), 6.74-6.40 (m, 3H), 4.84-4.67 (m, 1H), 3.78-3.68 (m, 2H), 2.99-2.91 (m, 1H), 2.73-2.66 (m, 3H), 2.36-2.26 (m, 1H), 2.04-1.97 (m, 3H), 1.76-1.48 (m, 10H), 1.26-1.24 (m, 6H), 1.16 (s, 3H), 1.07-0.94 (m, 3H).

### Example 11—Synthesis of Compound 11

### Synthetic Scheme

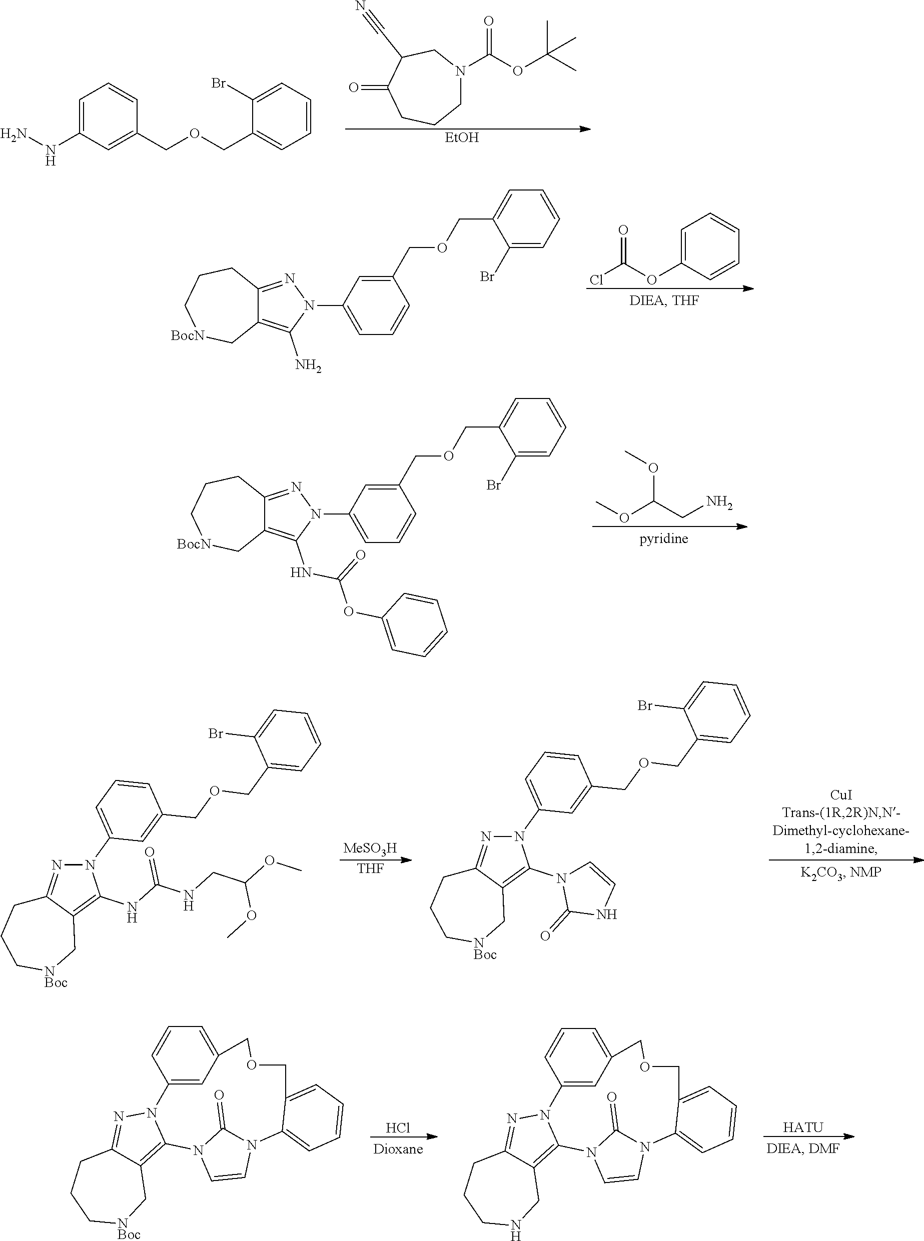

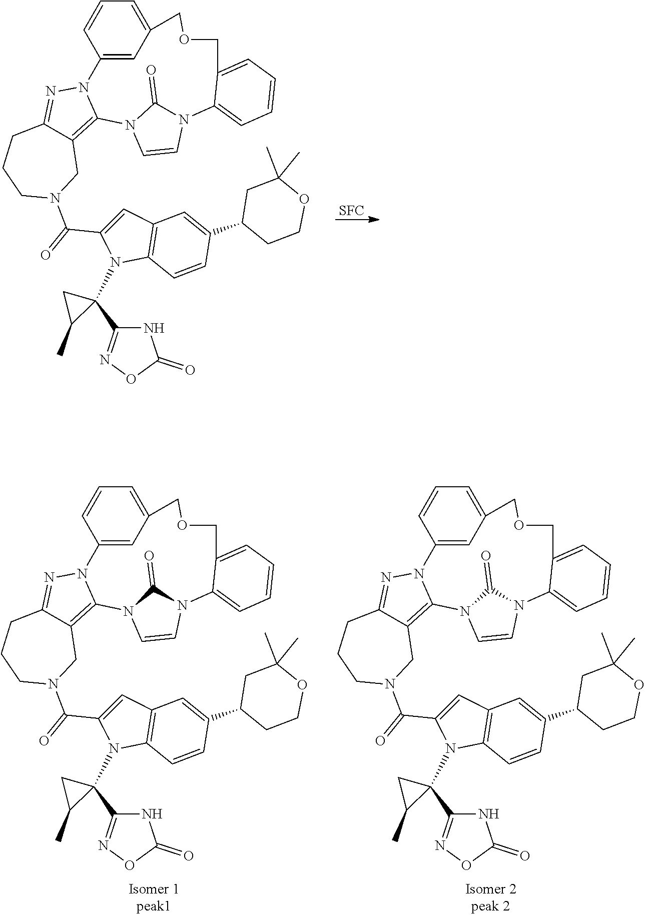

### Procedure for Synthesis of Compound 11

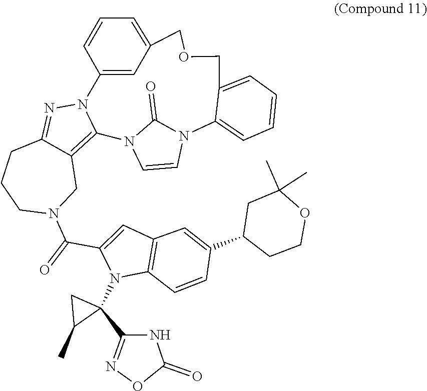 [SMILES: C[C@H]1C[C@]1(c1noc(=O)[nH]1)n1c(C(=O)N2CCCc3nn4c(c3C2)-n2ccn(c2=O)-c2ccccc2COCc2cccc-4c2)cc2cc([C@H]3CCOC(C)(C)C3)ccc21]

### 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-23-oxa-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaen-30-one

Step A: To a solution of (3-(((2-bromobenzyl)oxy)methyl)phenyl)hydrazine (0.7 g, 2.28 mmol, from Example 3, step C) in EtOH (14 mL) was added tert-butyl 3-cyano-4-oxo-1-azepanecarboxylate (543 mg, 2.28 mmol), and the reaction was stirred at 80° C. for 16 hr. The reaction mixture was concentrated under reduced pressure, and the residue was purified by column chromatography (0-40% EtOAc in PE) to give tert-butyl 3-amino-2-(3-(((2-bromobenzyl)oxy)methyl)phenyl)-2,6,7,8-tetrahydropyrazolo[4,3-c]azepine-5(4H)-carboxylate (950 mg, 1.8 mmol) as a yellow oil. m/z (ESI-pos) M+1=527.1/529.1

Step B: To a solution of tert-butyl 3-amino-2-(3-(((2-bromobenzyl)oxy)methyl)phenyl)-2,6,7,8-tetrahydropyrazolo[4,3-c]azepine-5(4H)-carboxylate (950 mg, 1.8 mmol) and DIPEA (698 mg, 5.4 mmol) in THF (38 mL) was added benzenyl chloroformate (564 mg, 3.6 mmol), and the reaction was stirred at 25° C. for 1 hr. The reaction mixture was concentrated under reduced pressure to give crude tert-butyl 2-(3-(((2-bromobenzyl)oxy)methyl)phenyl)-3-((phenoxycarbonyl)amino)-2,6,7,8-tetrahydropyrazolo[4,3-c]azepine-5(4H)-carboxylate (1.7 g, 1.58 mmol) as a yellow oil, which was used directly in the next step without further purification. m/z (ESI-pos) M+1=647.2/649.2

Step C: Crude tert-butyl 2-(3-(((2-bromobenzyl)oxy)methyl)phenyl)-3-((phenoxycarbonyl)amino)-2,6,7,8-tetrahydropyrazolo[4,3-c]azepine-5(4H)-carboxylate (1.7 g, 2.63 mmol) was dissolved in pyridine (34 mL), to which 2,2-dimethoxyethylamine (828 mg, 7.88 mmol) was added. The mixture was stirred at 25° C. for 3 hr. The reaction mixture was concentrated under reduced pressure. The crude compound was purified by column chromatography (0-65% EtOAc in PE) to give tert-butyl 2-(3-(((2-bromobenzyl)oxy)methyl)phenyl)-3-(3-(2,2-dimethoxyethyl)ureido)-2,6,7,8-tetrahydropyrazolo[4,3-c]azepine-5(4H)-carboxylate (0.7 g, 1.06 mmol) as a yellow oil. m/z (ESI-pos) M+1=658.1/660.1

Step D: To a solution of tert-butyl 2-(3-(((2-bromobenzyl)oxy)methyl)phenyl)-3-(3-(2,2-dimethoxyethyl)ureido)-2,6,7,8-tetrahydropyrazolo[4,3-c]azepine-5(4H)-carboxylate (0.7 g, 1.06 mmol) in THF (14 mL) was added methanesulfonic acid (102 mg, 1.06 mmol). The mixture was stirred at 60° C. for 1 hr. Aqueous K₃PO₄ was added to adjust the pH to ~9, and Boc₂O (232 mg, 1.06 mmol) was added and the resulting mixture was stirred at 25° C. for 1 hr. The reaction mixture was diluted with ethyl acetate (50 mL), washed with water (25 mL×2) and the aqueous layer was extracted with ethyl acetate (25 mL×2). The combined organic layers were washed with 1N aq. HCl (20 mL), then brine (20 mL) and dried over anhydrous Na₂SO₄. The mixture was filtered and concentrated under reduced pressure. The crude residue was purified by column chromatography (0-80% EtOAc in PE) to give product tert-butyl 2-(3-(((2-bromobenzyl)oxy)methyl)phenyl)-3-(2-oxo-2,3-dihydro-1H-imidazol-1-yl)-2,6,7,8-tetrahydropyrazolo[4,3-c]azepine-5(4H)-carboxylate (0.5 g, 841 μmol) as a yellow solid. m/z (ESI-pos) M+1=594.2/596.2

Step E: To a solution of tert-butyl 2-(3-(((2-bromobenzyl)oxy)methyl)phenyl)-3-(2-oxo-2,3-dihydro-1H-imidazol-1-yl)-2,6,7,8-tetrahydropyrazolo[4,3-c]azepine-5(4H)-carboxylate (0.3 g, 505 μmol) in NMP (15 mL) was added (1R,2R)-1,2-bis(methylamino)cyclohexane (108 mg, 757 μmol), copper iodide (48.1 mg, 252 μmol) and K₂CO₃ (209 mg, 1.51 mmol). The reaction mixture was heated at 130° C. for 3 hr under N₂. The reaction mixture was cooled to ambient temperature and diluted with ethyl acetate (50 mL), washed with water (25 mL×2) and the aqueous layer was extracted with ethyl acetate (25 mL×2). The combined organic layers were washed with aqueous LiCl solution (20 mL) and dried over anhydrous Na₂SO₄. The mixture was then filtered and concentrated under reduced pressure. The residue was purified by column chromatography (0-40% EtOAc in PE) to give tert-butyl 30-oxo-23-oxa-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaene-8-carboxylate (0.2 g, 389 μmol) as a yellow oil. m/z (ESI-pos) M+1=514.2

Step F: tert-butyl 30-oxo-23-oxa-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaene-8-carboxylate (0.2 g, 389 μmol) was dissolved in HCl/1,4-dioxane (4M, 1 mL) and stirred at 25° C. for 1 h. The reaction mixture was concentrated to give 23-oxa-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaen-30-one hydrogen chloride (0.2 g, 387 μmol) as a yellow solid, which was used directly in the next step. m/z (ESI-pos) M+1=414.1

Step G: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (199 mg, 484 μmol) in DMF (20 mL) was added HATU (221 mg, 580 μmol) and DIPEA (188 mg, 1.45 mmol). The mixture was stirred at ambient temperature for 10 min, then 23-oxa-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaen-30-one hydrogen chloride (0.2 g, 484 μmol) was added to the mixture. The mixture was stirred overnight. The reaction mixture was diluted with ethyl acetate (50 mL), washed with water (25 mL×2) and the aqueous layer was extracted with ethyl acetate (25 mL×2). The combined organic layers were washed with brine (20 mL), dried over anhydrous Na₂SO₄, filtered and concentrated under reduced pressure. The residue was purified by column chromatography (0-80% EtOAc in PE) to give 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-23-oxa-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaen-30-one (160 mg, 0.198 mmol) as a white solid. m/z (ESI-pos) M+1=807.3

Step H: 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-23-oxa-2,3,8,12,15-pentaazahexacyclo[23.3.1.1¹²,¹⁵.0²,¹¹.0⁴,¹⁰.0¹⁶,²¹]triaconta-1(29),3,10,13,16,18,20,25,27-nonaen-30-one (160 mg, 0.2 mmol) was dissolved in MeOH (200 mL) and separated by chiral supercritical fluid chromatography (separation condition: Daicel CHIRALCEL OJ, 250 mm*30 mm I.D., 10 μm; CO2/MeOH [0.2% NH₃ (7M Solution in MeOH)]=45/55 at 70 g/min; Temp: 35° C.; Wavelength: 214 nm) to afford Isomer 1 (50 mg, purity: 90%) and Isomer 2 (60 mg, purity: 90%), which was further purified by prep-HPLC [Instrument: Waters MS-triggered Prep-LC with SQD2 detector; Column: Xbridge 5 u C18 150×19 mm; Gradient: eluted with 72-100% ACN in H₂O (0.1% FA) in 9.5 min; Flow rate: 20 mL/min] to give Isomer 1 (17.56 mg, 35% yield) and Isomer 2 (12.7 mg, 21.6% yield) as white solids (The absolute configuration was unknown, the configuration of two atropisomers were assigned arbitrarily). Isomer 1: m/z (ESI-pos) M+1=807.3; ¹H NMR (400 MHz, DMSO) δ 11.60 (s, 1H), 7.59-7.37 (m, 6H), 7.25 (dd, J=7.6 Hz, 4H), 6.91 (s, 1H), 6.79 (s, 1H), 6.55 (s, 2H), 4.73-4.50 (m, 3H), 4.18 (s, 1H), 4.00 (d, J=9.6 Hz, 2H), 3.68 (s, 3H), 3.05 (s, 1H), 3.02-2.90 (m, 2H), 2.02 (d, J=10.8 Hz, 2H), 1.69-1.54 (m, 7H), 1.21 (d, J=41.6 Hz, 8H), 0.93 (d, J=56.0 Hz, 2H). Isomer 2: m/z (ESI-pos) M+1=807.3; ¹H NMR (400 MHz, DMSO) δ 11.55 (s, 1H), 7.60-7.41 (m, 6H), 7.33 (d, J=8.0 Hz, 1H), 7.21 (dd, J=7.6 Hz, 3H), 6.92 (s, 1H), 6.74 (s, 1H), 6.50 (d, J=92.0 Hz, 2H), 4.97-4.70 (m, 3H), 4.50 (d, J=13.6 Hz, 1H), 4.27 (s, 1H), 3.99 (d, J=8.8 Hz, 1H), 3.69 (d, J=6.0 Hz, 3H), 3.03-2.89 (m, 3H), 2.10-1.98 (m, 2H), 1.85-1.34 (m, 7H), 1.30-0.99 (m, 10H).

### Example 12—Synthetic of Compound 12

### Synthetic Scheme

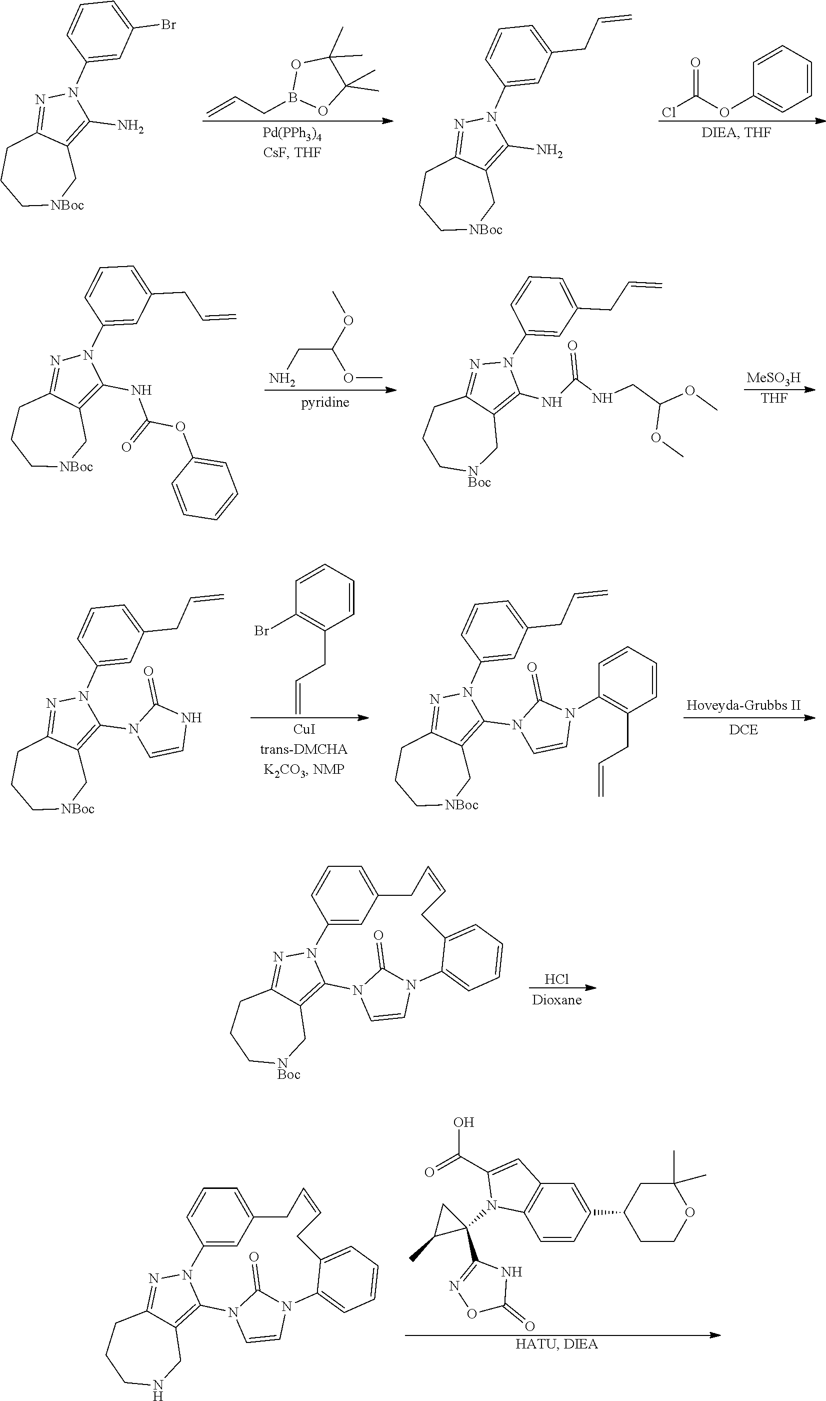

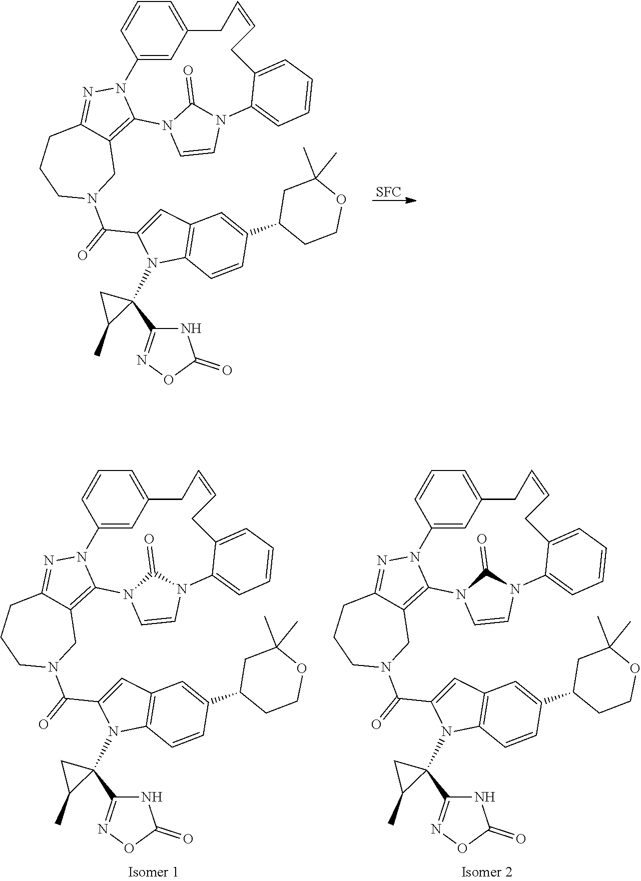

### Procedure for Synthesis of Compound 12

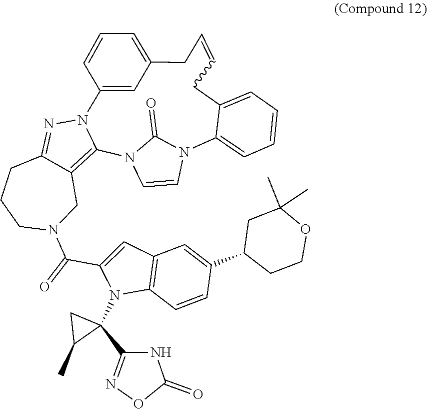 [SMILES: C[C@H]1C[C@]1(c1noc(=O)[nH]1)n1c(C(=O)N2CCCc3nn4c(c3C2)-n2ccn(c2=O)-c2ccccc2CC=CCc2cccc-4c2)cc2cc([C@H]3CCOC(C)(C)C3)ccc21]

### 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,23,26,28-decaen-31-one

Step A: To a mixture of tert-butyl 3-amino-2-(3-bromophenyl)-2,6,7,8-tetrahydropyrazolo[4,3-c]azepine-5(4H)-carboxylate (2.2 g, 5.4 mmol, from Example 8, Step A), 2-allyl-4,4,5,5-tetramethyl-1,3,2-dioxaborolane (1.36 g, 8.1 mmol) and CsF (2.46 g, 16.2 mmol) in THF (50 mL) was added Pd(PPh₃)₄ (312 mg, 0.27 mmol) at 25° C. under N₂ protection. The resulting mixture was warmed to 80° C. where it stirred for 16 hr. The reaction mixture was cooled down to room temperature, diluted with ethyl acetate (50 mL), and washed with water (50 mL×3). The aqueous layer was extracted with ethyl acetate (30 mL×3). The combined organic layers were washed with brine (50 mL×3), dried over anhydrous Na₂SO₄, filtered and concentrated under reduced pressure to give the crude compound. Then the crude compound was purified by column chromatography (0-60% EtOAc in PE) to give tert-butyl 3-amino-2-(3-vinylphenyl)-2,6,7,8-tetrahydropyrazolo[4,3-c]azepine-5(4H)-carboxylate (2.2 g, 4.72 mmol) as a yellow oil. m/z (ESI-pos) M+1=369.3

Step B: To a solution of tert-butyl 3-amino-2-(3-vinylphenyl)-2,6,7,8-tetrahydropyrazolo[4,3-c]azepine-5(4H)-carboxylate (2.0 g, 5.43 mmol), DIEA (2.1 g, 16.3 mmol) in Tetrahydrofuran (15 mL) was added benzenyl chloroformate (1.7 g, 10.9 mmol) at 0° C. The reaction mixture was then allowed to warm to 30° C. where it stirred for 2 hr. The reaction mixture was concentrated to give crude tert-butyl 2-(3-allylphenyl)-3-((phenoxycarbonyl)amino)-2,6,7,8-tetrahydropyrazolo[4,3-c]azepine-5(4H)-carboxylate (2.1 g crude) as a yellow oil, which was used directly without any further purification. m/z (ESI-pos) M+1=489.3

Step C: Crude tert-butyl 2-(3-allylphenyl)-3-((phenoxycarbonyl)amino)-2,6,7,8-tetrahydropyrazolo[4,3-c]azepine-5(4H)-carboxylate (2.1 g crude, 5.43 mmol) was dissolved in pyridine (50 mL), to which 2,2-dimethoxyethylamine (1.8 g, 17.1 mmol) was added. The mixture was stirred at 25° C. for 3 hr. The reaction mixture was concentrated under reduced pressure. The residue was purified by column chromatography (0-60% EtOAc in PE) to give tert-butyl 2-(3-allylphenyl)-3-(3-(2,2-dimethoxyethyl)ureido)-2,6,7,8-tetrahydropyrazolo[4,3-c]azepine-5(4H)-carboxylate (1.5 g, 3.00 mmol) as a yellow solid. m/z (ESI-pos) M+1=500.3

Step D: To a solution of tert-butyl 2-(3-allylphenyl)-3-(3-(2,2-dimethoxyethyl)ureido)-2,6,7,8-tetrahydropyrazolo[4,3-c]azepine-5(4H)-carboxylate (1.5 g, 3.00 mmol) in THF (60 mL) was added methanesulfonic acid (346 mg, 3.6 mmol) at ambient temperature. The mixture was stirred at 60° C. for 2 hr. The mixture weas cooled to ambient temperature and then aqueous K₃PO₄ was added to adjust pH to ~9, and then Boc₂O (654 mg, 3.00 mmol) was added. The resulting mixture was stirred at 25° C. for 16 hr and then diluted with ethyl acetate (50 mL), washed with water (30 mL×3) and the aqueous layer was extracted with ethyl acetate (30 mL×3). The combined organic layers were washed with brine (50 mL) and dried over anhydrous Na₂SO₄, filtered and concentrated under reduced pressure. The crude compound was purified by column chromatography (0-100% EtOAc in PE) to give tert-butyl 2-(3-allylphenyl)-3-(2-oxo-2,3-dihydro-1H-imidazol-1-yl)-2,6,7,8-tetrahydropyrazolo[4,3-c]azepine-5(4H)-carboxylate (1.0 g, 2.07 mmol) as a yellow solid. m/z (ESI-pos) M+1=436.2

Step E: To a solution of tert-butyl 2-(3-allylphenyl)-3-(2-oxo-2,3-dihydro-1H-imidazol-1-yl)-2,6,7,8-tetrahydropyrazolo[4,3-c]azepine-5(4H)-carboxylate (650 mg, 1.49 mmol) and 1-allyl-2-bromobenzene (382 mg, 1.94 mmol) in N-methyl-2-pyrrolidone (10 mL) were added (1R,2R)-1,2-bis(methylamino)cyclohexane (212 mg, 1.49 mmol), CuI (47 mg, 0.746 mmol) and K₂CO₃ (618 mg, 4.48 mmol). The reaction mixture was heated at 130° C. for 3 hr under the N₂. The reaction mixture was cooled to ambient temperature and diluted with ethyl acetate (50 mL), washed with water (30 mL×3) and the aqueous layer was extracted with ethyl acetate (30 mL×3). The combined organic layers were washed with brine (50 mL), dried over anhydrous Na₂SO₄, filtered and concentrated under reduced pressure to give the crude compound, which was purified by column chromatography (0-100% EtOAc in PE) to give tert-butyl 2-(3-allylphenyl)-3-(3-(2-allylphenyl)-2-oxo-2,3-dihydro-1H-imidazol-1-yl)-2,6,7,8-tetrahydropyrazolo[4,3-c]azepine-5(4H)-carboxylate (670 mg) as a yellow solid. m/z (ESI-pos) M+1=552.3

Step F: To a solution of tert-butyl 2-(3-allylphenyl)-3-(3-(2-allylphenyl)-2-oxo-2,3-dihydro-1H-imidazol-1-yl)-2,6,7,8-tetrahydropyrazolo[4,3-c]azepine-5(4H)-carboxylate (670 mg, 1.21 mmol) in DCE (670 mL) was added Hoveyda-Grubbs II (670 mg, 1.07 mmol) at 25° C. under N₂ atmosphere. The reaction mixture was heated at 80° C. for 3 hr. The reaction mixture was cooled to ambient temperature and quenched with water (300 mL) and the aqueous layer was extracted with dichloromethane (50 mL×3). The combined organic layers were washed with brine (350 mL), dried over anhydrous Na₂SO₄, filtered and concentrated under reduced pressure to give the crude compound. The crude compound was purified by column chromatography (0-100% EA in PE) to give tert-butyl 31-oxo-2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,23,26,28-decaene-8-carboxylate (510 mg) as a yellow solid. m/z (ESI-pos) M+1=524.3

Step G: tert-butyl 31-oxo-2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,23,26,28-decaene-8-carboxylate (160 mg, 0.306 mmol) was dissolved in HCl/1,4-dioxane (4M, 6 mL) and stirred at 25° C. for 1 h. The reaction mixture was concentrated under reduced pressure to give 2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,23,26,28-decaen-31-one hydrogen chloride (180 mg crude, 0.306 mmol) as a yellow solid, which was used directly to next step. m/z (ESI-pos) M+1=424.2

Step H: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (125 mg, 0.306 mmol) in DMF (5 mL) was added DIEA (394 mg, 3.06 mmol) and HATU (174 mg, 0.459 mmol) at 25° C., the mixture was stirred at ambient temperature for 10 min, then 2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,23,26,28-decaen-31-one hydrogen chloride (180 mg crude, 0.306 mmol) was added to the mixture. The mixture was stirred overnight and then diluted with ethyl acetate (30 mL) and washed with water (30 mL×2). The aqueous layer was extracted with ethyl acetate (30 mL×2). The combined organic layers were washed with brine (30 mL), dried over anhydrous Na₂SO₄, filtered and concentrated under reduced pressure to give the crude compound which was purified by prep-HPLC [Waters MS-triggered Prep-LC with QDA detector, WELCH Xtimate C18 21.2*250 mm 10 μm, eluted with 60-100% ACN in H₂O (0.1% FA)] to give 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,23,26,28-decaen-31-one (60 mg) as a yellow solid. m/z (ESI-pos) M+1=817.3

Step I: 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,23,26,28-decaen-31-one (60 mg, 0.07 mmol) was dissolved in MeOH (150 mL) and separated by chiral supercritical fluid chromatography [separation condition: Daicel CHIRALPAK OZ_3, 3*150 mm, 3 um; Mobile Phase: CO2/MeOH (0.1% DEA)=45/55 at 1.5 mL/min; Temp: 37° C.; Wavelength: 254 nm] to afford Isomer 1 (34.55 mg) and Isomer 2 (16.15 mg) as white solids (The absolute configuration was unknown, the configuration of two atropisomers were assigned arbitrarily).

Isomer 1: m/z (ESI-pos) M+1=817.3; ¹H NMR (400 MHz, DMSO) δ 10.60 (s, 1H), 7.58-7.46 (m, 2H), 7.35-7.14 (m, 8H), 7.07-7.01 (m, 2H), 6.87-6.74 (m, 2H), 6.61-6.42 (m, 2H), 5.45-5.32 (m, 1H), 5.28-5.16 (m, 1H), 4.91-4.56 (m, 2H), 4.11-3.87 (m, 2H), 3.71-3.59 (m, 2H), 3.34-3.14 (m, 3H), 2.96-2.87 (m, 3H), 2.02-1.89 (m, 2H), 1.63-1.49 (m, 5H), 1.30-1.22 (m, 6H), 1.18-1.12 (m, 6H).

Isomer 2: m/z (ESI-pos) M+1=817.3; ¹H NMR (400 MHz, DMSO) δ 11.48 (s, 1H), 7.48-7.27 (m, 9H), 7.06 (d, J=7.6 Hz, 1H), 6.91-6.61 (m, 4H), 5.54-5.37 (m, 1H), 5.22-5.17 (m, 1H), 4.71-4.43 (m, 2H), 3.72-3.61 (m, 4H), 3.27-3.19 (m, 3H), 3.00-2.91 (m, 3H), 2.08-1.99 (m, 2H), 1.65-1.40 (m, 8H), 1.22 (s, 3H), 1.13 (s, 3H), 1.03 (s, 3H).

### Example 13—Synthesis of Compound 13

### Synthetic Scheme

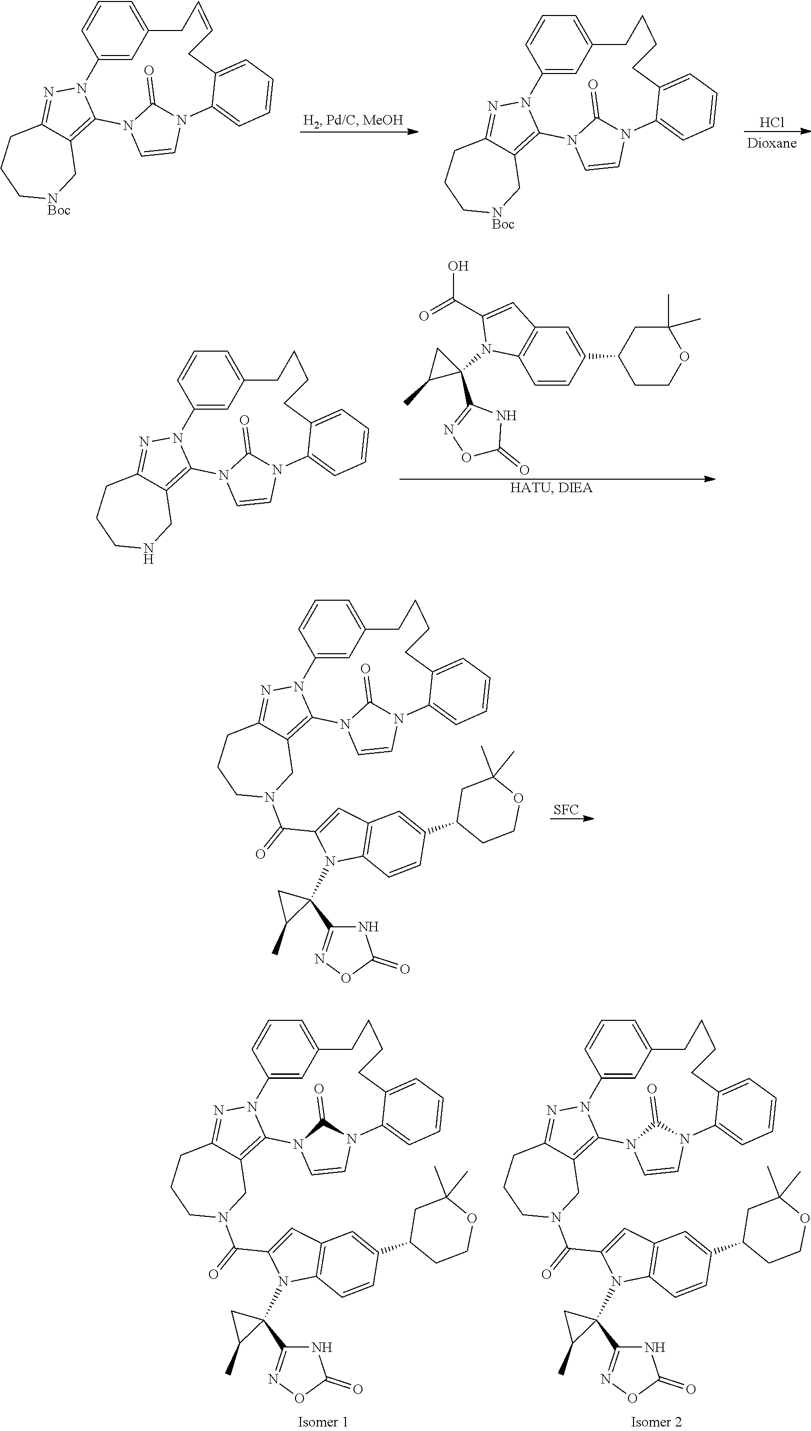

### Procedure for Synthesis of Compound 13

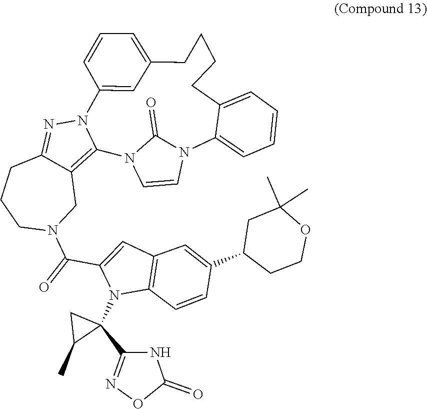 [SMILES: C[C@H]1C[C@]1(c1noc(=O)[nH]1)n1c(C(=O)N2CCCc3nn4c(c3C2)-n2ccn(c2=O)-c2ccccc2CCCCc2cccc-4c2)cc2cc([C@H]3CCOC(C)(C)C3)ccc21]

### 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-2,3,8,12,15-pentaazahexacyclo[24.3.1.1¹²,¹⁵.0²,¹¹.0⁴,¹⁰.0¹⁶,²¹]hentriaconta-1(30), 3,10,13,16,18,20,26,28-nonaen-31-one

Step A: To a solution of tert-butyl 31-oxo-2,3,8,12,15-pentaazahexacyclo[24.3.1.1¹²,¹⁵.0²,¹¹.0⁴,¹⁰.0¹⁶,²¹]hentriaconta-1(30), 3,10,13,16,18,20,23,26,28-decaene-8-carboxylate (210 mg, 0.40 mmol) in MeOH (10 mL) was added Pd/C (105 mg, 50% w/w) at 25° C. The mixture was evacuated and backfilled with H₂, then stirred at 25° C., under H₂ atmosphere for 16 hr. The reaction mixture was filtered through a pad of celite and the filtrate was evaporated under reduced pressure to give tert-butyl 31-oxo-2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,26,28-nonaene-8-carboxylate (200 mg, 0.36 mmol) as a yellow solid. m/z (ESI-pos) M+1=526.2

Step B: tert-butyl 31-oxo-2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,26,28-nonaene-8-carboxylate (200 mg, 0.38 mmol) was dissolved in HCl/1,4-dioxane (4M, 6 mL) and stirred at 25° C. for 1 h. The reaction mixture was concentrated under reduced pressure to give 2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,26,28-nonaen-31-one hydrogen chloride (220 mg crude, 0.38 mmol) as a yellow solid. m/z (ESI-pos) M+1=426.3

Step C: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (156 mg, 0.38 mmol) in DMF (5 mL) was added DIEA (490 mg, 3.8 mmol) and HATU (216 mg, 0.56 mmol) at 25° C., the mixture was stirred at ambient temperature for 10 min, then 2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,26,28-nonaen-31-one hydrogen chloride (220 mg crude, 0.38 mmol) was added to the mixture. The mixture was stirred overnight. The reaction mixture was diluted with ethyl acetate (30 mL), washed with water (30 mL×2). The aqueous layer was extracted with ethyl acetate (30 mL×2). The combined organic layers were washed with brine (30 mL), dried over anhydrous Na₂SO₄, filtered and concentrated under reduced pressure to give the crude compound which was purified by prep-HPLC [Waters MS-triggered Prep-LC with QDA detector, WELCH Xtimate C18 21.2*250 mm 10 μm, eluted with 60-100% ACN in H₂O (0.1% FA)] to give 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,26,28-nonaen-31-one (100 mg) as a yellow solid. m/z (ESI-pos) M+1=819.4

Step D: 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,26,28-nonaen-31-one (100 mg, 0.12 mmol) was dissolved in MeOH (150 mL) and separated by chiral supercritical fluid chromatography (separation condition: Daicel CHIRALPAK OX_3, 3*150 mm, 3 um; Mobile Phase: CO₂/MeOH (0.1% DEA)=50/50 at 1.5 mL/min; Temp: 37° C.; Wavelength: 254 nm) to afford the Isomer 1(26.7 mg) and Isomer 2 (27.4 mg) as white solids (The absolute configuration was unknown, the configuration of two atropisomers were assigned arbitrarily).

Isomer 1: m/z (ESI-pos) M+1=819.4; ¹H NMR (400 MHz, DMSO) δ 11.29 (s, 1H), 7.43-7.12 (m, 10H), 6.98-6.65 (m, 4H), 4.86-4.66 (m, 2H), 4.04-3.67 (m, 4H), 2.91-2.65 (m, 1H), 2.73-2.65 (m, 1H), 2.04-1.98 (m, 2H), 1.69-1.47 (m, 9H), 1.30-1.23 (m, 8H), 1.17-1.08 (m, 6H), 1.04-0.93 (m, 2H).

Isomer 2: m/z (ESI-pos) M+1=819.3; ¹H NMR (400 MHz, DMSO) δ 10.04 (s, 1H), 7.43-7.15 (m, 10H), 6.83-6.61 (m, 4H), 4.97-4.77 (m, 2H), 4.02-3.70 (m, 4H), 2.93-2.88 (m, 1H), 2.72-2.68 (m, 1H), 2.06-2.00 (m, 2H), 1.71-1.49 (m, 9H), 1.32-1.25 (m, 8H), 1.18-1.15 (m, 6H), 1.09-1.06 (m, 2H).

### Example 14—Synthesis of Compound 14

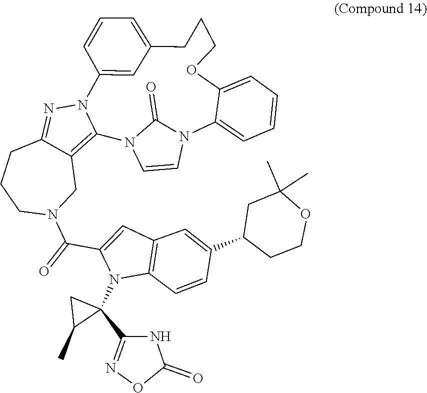 [SMILES: C[C@H]1C[C@]1(c1noc(=O)[nH]1)n1c(C(=O)N2CCCc3nn4c(c3C2)-n2ccn(c2=O)-c2ccccc2OCCCc2cccc-4c2)cc2cc([C@H]3CCOC(C)(C)C3)ccc21]

### 8-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-22-oxa-2,3,8,12,15-pentaazahexacyclo[24.3.1.1¹²,¹⁵.0²,¹¹.0⁴,¹⁰.0¹⁶,²¹]hentriaconta-1(30),3,10,13,16,18,20,26,28-nonaen-31-one

Compound 14 was synthesized following the procedure of Example 7, with tert-butyl (2S)-3-cyano-2-methyl-4-oxo-1-piperidinecarboxylate being replaced with tert-butyl 3-cyano-4-oxo-1-azepanecarboxylate.

Isomer 1: m/z (ESI-pos) M+1=821.3; ¹H NMR (400 MHz, DMSO) δ 11.59 (s, 1H), 7.47-7.30 (m, 6H), 7.25-7.20 (m, 1H), 7.15-7.10 (m, 2H), 6.99-6.47 (m, 5H), 5.15-4.46 (m, 3H), 3.85-3.69 (m, 5H), 3.01-2.93 (m, 2H), 2.77-2.68 (m, 2H), 2.04-2.01 (m, 4H), 1.68-1.49 (m, 7H), 1.26-1.23 (m, 4H), 1.15-0.99 (m, 6H).

Isomer 2: m/z (ESI-pos) M+1=821.3; ¹H NMR (400 MHz, DMSO) δ 11.60 (s, 1H), 7.47-7.30 (m, 5H), 7.25-7.13 (m, 4H), 6.98-6.88 (m, 2H), 6.77-6.47 (m, 3H), 4.81-4.26 (m, 3H), 3.88-3.69 (m, 5H), 3.02-2.95 (m, 2H), 2.79-2.71 (m, 2H), 2.05-2.01 (m, 4H), 1.69-1.49 (m, 7H), 1.27-1.23 (m, 4H), 1.15-1.00 (m, 6H).

### Example 15—Synthesis of Compound 15

### Synthetic Scheme

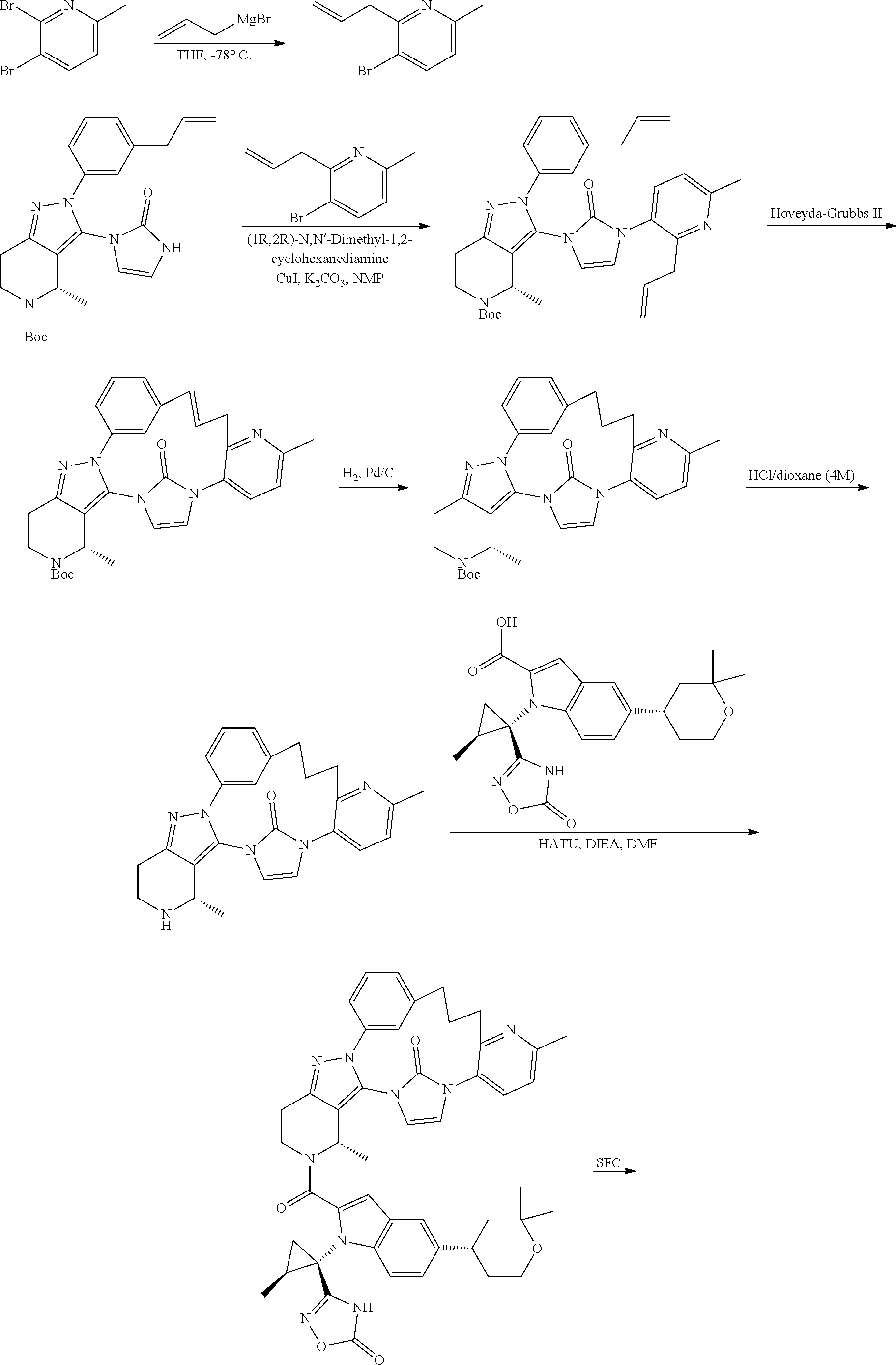

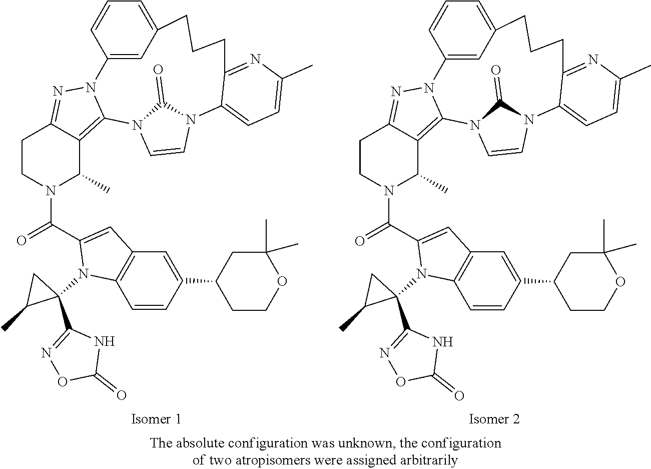

### Procedure for Synthesis of Compound 15

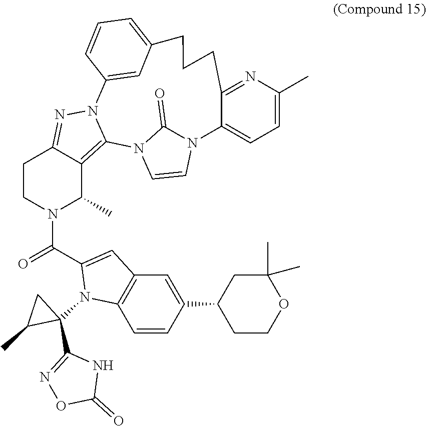

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8,18-dimethyl-2,3,7,11,14,19-hexaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of 2,3-dibromo-6-methylpyridine (4.6 g, 18.3 mmol) in tetrahydrofuran (50 mL) was added allylmagnesium bromide (2.8 g, 19.2 mmol, 1.0 mol/L in diethyl ether) at −78° C. N₂ protection. The resulting mixture was allowed to warm to rt and stirred at ambient temperature for 16 hr. The reaction was quenched with water and diluted with ethyl acetate, washed with water (3×). The aqueous layer was extracted with ethyl acetate (3×). The combined organic layers were washed with brine solution (3×), dried over anhydrous Na₂SO₄, filtered and concentrated to give the crude compound. The crude compound was purified by column chromatography (0-50% EtOAc in PE) to give 2-allyl-3-bromo-6-methylpyridine (3.3 g, 14 mmol, 76% yield) as a brown oil. ¹H NMR (400 MHz, DMSO) δ 7.88 (d, J=8.4 Hz, 1H), 7.08 (d, J=8.0 Hz, 1H), 6.03-5.94 (m, 1H), 5.10-5.04 (m, 2H), 3.63-3.60 (m, 2H), 2.42 (s, 3H).

Step B: To a solution of tert-butyl (S)-2-(3-allylphenyl)-4-methyl-3-(2-oxo-2,3-dihydro-1H-imidazol-1-yl)-2,4,6,7-tetrahydro-5H-pyrazolo[4,3-c]pyridine-5-carboxylate (1100 mg, 2.52 mmol) (See Example 4) and 1-allyl-2-bromobenzene (533 mg, 2.52 mmol) in N-methyl-2-pyrrolidone (10 mL) were added (1R,2R)-1,2-bis(methylamino)cyclohexane (429 mg, 3.02 mmol), CuI (480 mg, 2.52 mmol) and K₂CO₃ (1043 mg, 7.56 mmol). The reaction mixture was heated at 130° C. for 3 hr under a N₂ atmosphere. The reaction mixture was cooled to ambient temperature and was diluted with ethyl acetate, washed with water (3×) and the aqueous layer was back extracted with ethyl acetate (3×). The combined organic layers were washed with brine solution (3×), dried over anhydrous Na₂SO₄ and evaporated under reduced pressure to give the crude compound. The crude compound was purified by column chromatography (0-100% EtOAc in PE) to give tert-butyl (S)-3-(3-(2-allyl-6-methylpyridin-3-yl)-2-oxo-2,3-dihydro-1H-imidazol-1-yl)-2-(3-allylphenyl)-4-methyl-2,4,6,7-tetrahydro-5H-pyrazolo[4,3-c]pyridine-5-carboxylate (660 mg, 41% yield) as a yellow solid. m/z (ESI-pos) M+1=567.3

Step C: To a solution of tert-butyl (S)-3-(3-(2-allyl-6-methylpyridin-3-yl)-2-oxo-2,3-dihydro-1H-imidazol-1-yl)-2-(3-allylphenyl)-4-methyl-2,4,6,7-tetrahydro-5H-pyrazolo[4,3-c]pyridine-5-carboxylate (500 mg, 0.88 mmol) in DCE (500 mL) was added Hoveyda-Grubbs II (500 mg, 0.8 mmol) and acetic acid (1.2 g, 6.34 mmol) at 25° C. under N₂. The reaction mixture was heated at 80° C. for 2 hr. The reaction mixture was cooled to ambient temperature and quenched with water and the aqueous layer was extracted with dichloromethane (3×). The combined organic layers were washed with brine, dried over anhydrous Na₂SO₄ and concentrated. The crude compound was purified by column chromatography (0-100% EtOAc in PE) to give tert-butyl (S)-8,18-dimethyl-29-oxo-2,3,7,11,14,19-hexaazahexacyclo[22.3.1.1¹¹,¹⁴.0²,¹⁰.0⁴,⁹.0¹⁵,²⁰]nonacosa-1(28),3,9,12,15,17,19,22,24,26-decaene-7-carboxylate (160 mg) as a yellow solid. m/z (ESI-pos) M+1=525.2

Step D: To a solution of tert-butyl (S)-8,18-dimethyl-29-oxo-2,3,7,11,14,19-hexaazahexacyclo[22.3.1.1¹¹,¹⁴.0²,¹⁰.0⁴,⁹.0¹⁵,²⁰]nonacosa-1(28),3,9,12,15,17,19,22,24,26-decaene-7-carboxylate (130 mg, 0.41 mmol) in tetrahydrofuran (10 mL) was added Pd/C (65 mg, 50% w/w) at 25° C. The mixture was then stirred under a H₂ atmosphere at 25° C. for 16 hr. The reaction mixture was filtered and the filtrate was concentrated to give crude tert-butyl (S)-8,18-dimethyl-29-oxo-2,3,7,11,14,19-hexaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (130 mg) as a yellow solid. m/z (ESI-pos) M+1=527.2.

Step E: tert-butyl (S)-8,18-dimethyl-29-oxo-2,3,7,11,14,19-hexaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (130 mg, 0.15 mmol) was dissolved in HCl/1,4-dioxane (4M, 6 mL) and stirred at 25° C. for 1 h. The reaction mixture was directly concentrated under reduced pressure to give (S)-8,18-dimethyl-2,3,7,11,14,19-hexaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (120 mg, 0.25 mmol) as a yellow solid. m/z (ESI-pos) M+1=427.1.

Step F: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (116 mg, 0.28 mmol) in dimethylformamide (6 mL) was added DIEA (364 mg, 2.81 mmol) and HATU (160 mg, 042 mmol), the mixture stirred at ambient temperature for 10 min, then(S)-8,18-dimethyl-2,3,7,11,14,19-hexaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (120 mg crude, 0.28 mmol) was added to the mixture. The mixture was stirred at ambient temperature overnight. The reaction mixture was diluted with ethyl acetate, washed with water (3×). The aqueous layer was extracted with ethyl acetate (3×). The combined organic layers were washed with brine solution, dried over anhydrous Na₂SO₄, filtered and concentrated to give the crude compound which was purified by pre-HPLC [Waters MS-triggered Prep-LC with QDA detector, WELCH Xtimate C18 21.2*250 mm 10 μm, eluted with 60-100% ACN in H₂O (0.1% FA)] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8,18-dimethyl-2,3,7,11,14,19-hexaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (40 mg) as a yellow solid. The material was then was dissolved in Hex/EtOH (130 mL) and separated by chiral supercritical fluid chromatography (Column: Daicel CHIRALCEL IB N-5, 250 mm×30 mm I.D., 10 μm; Mobile Phase: Hex/EtOH [0.2% NH₃ (7M Solution in MeOH)]=50/50 at 30 mL/min; Temp: 35° C.; Wavelength: 214 nm) to afford the Isomer 1 (19.58 mg) and Isomer 2 (11.32 mg) as white solids (The absolute configuration was unknown, the configuration of two atropisomers were assigned arbitrarily).

Isomer 1: m/z (ESI-pos) M+1=820.3. ¹H NMR (400 MHz, DMSO) δ 11.30 (s, 1H), 7.66 (d, J=8.0 Hz, 1H), 7.50 (s, 1H), 7.43-7.39 (m, 3H), 7.23-7.15 (m, 4H), 6.85-6.78 (m, 1H), 6.67-6.55 (m, 2H), 5.69-5.52 (m, 1H), 4.51-4.28 (m, 1H), 3.73-3.71 (m, 2H), 3.60-3.48 (m, 1H), 2.91-2.75 (m, 4H), 2.38-2.21 (m, 2H), 2.02-1.94 (m, 2H), 1.71-1.49 (m, 9H), 1.36-1.16 (m, 14H).

Isomer 2: m/z (ESI-pos) M+1=820.3. ¹H NMR (400 MHz, DMSO) δ 7.75 (d, J=6.4 Hz, 1H), 7.46-7.38 (m, 4H), 7.24-7.15 (m, 4H), 6.81-6.75 (m, 1H), 6.62-6.57 (m, 2H), 5.66-5.57 (m, 1H), 4.43-4.28 (m, 1H), 3.76-3.70 (m, 2H), 3.58-3.50 (m, 1H), 2.94-2.71 (m, 4H), 2.34-2.25 (m, 2H), 2.05-1.90 (m, 2H), 1.70-1.52 (m, 9H), 1.36-1.15 (m, 14H).

### Example 16—Synthesis of Compound 16

### Synthetic Scheme

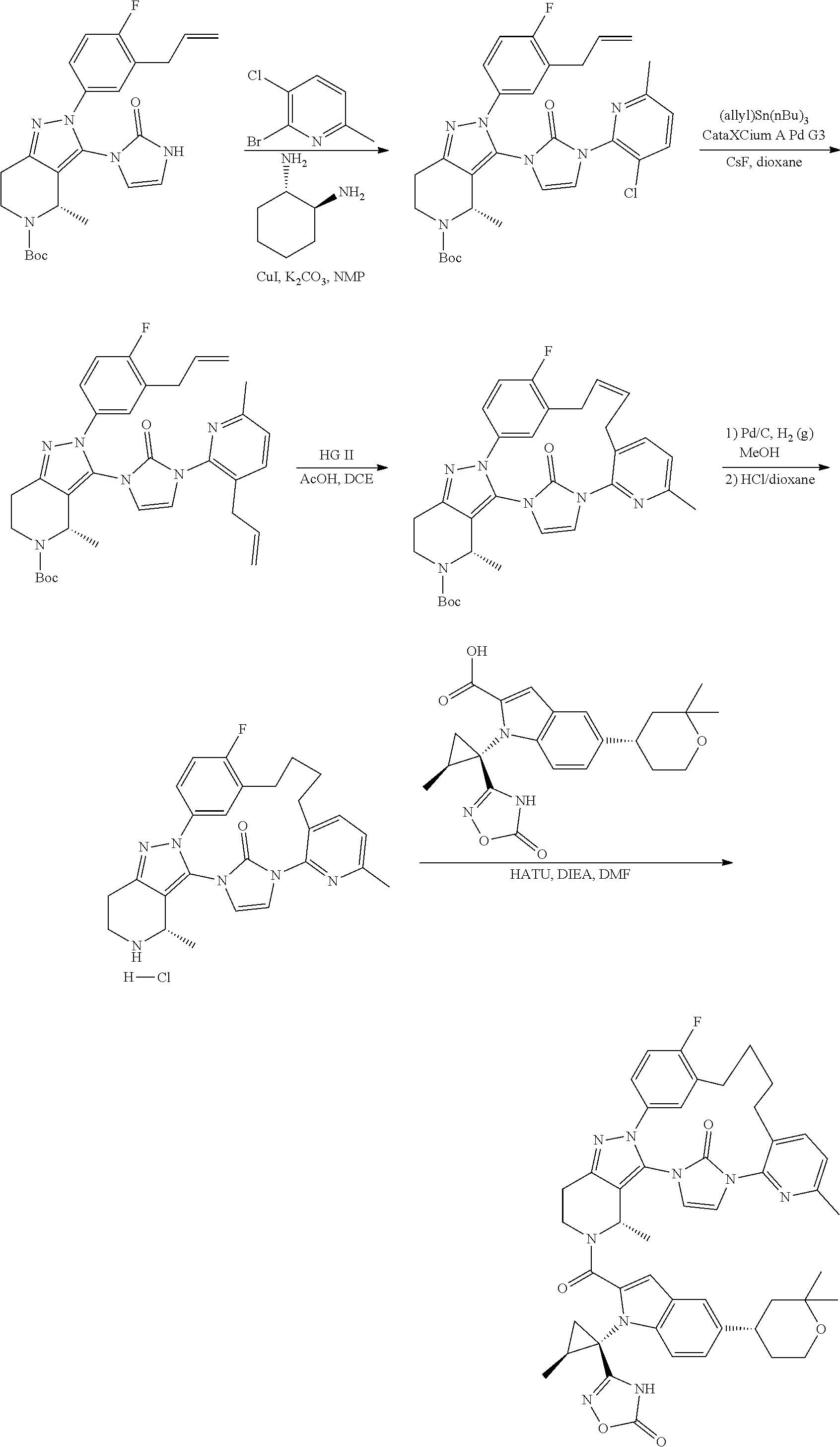

### Procedure for Synthesis of Compound 16

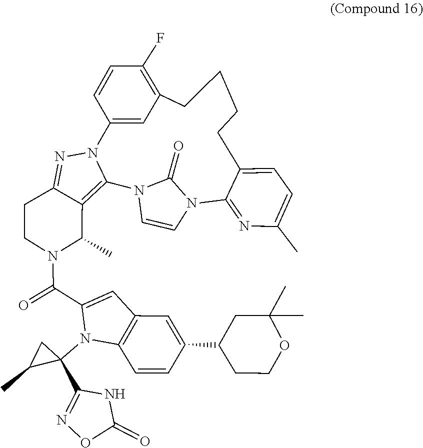

### (S)-7-({5-[(R)-2,2-dimethyltetrahydro-4H-pyran-4-yl]-1-[(2S)-2-methyl-1-(5-oxo-4,5-dihydro-1,2,4-oxadiazol-3-yl)cyclopropyl]-2-indolyl}carbonyl)-26-fluoro-8,17-dimethyl-2,3,7,11,14,16-hexaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: A solution of tert-butyl (S)-2-(3-allyl-4-fluorophenyl)-4-methyl-3-(2-oxo-2,3-dihydro-1H-imidazol-1-yl)-1,2,5-triaza-2,4,6,7-tetrahydro-5H-indene-5-carboxylate (120 mg, 265 μmol), 2-bromo-3-chloro-6-methylpyridine (81.9 mg, 397 μmol) (Obtained from following a similar procedure to that shown for Compound 1), CuI (25.2 mg, 132 μmol), (1S,2S)-1,2-bis(methylamino)cyclohexane (33.9 mg, 238 μmol), and K₂CO₃ (110 mg, 794 μmol) in 0.6 mL of NMP was placed under vacuumed and then backfilled with nitrogen and heated to 130° C. for 9 hours. The solution was then taken up in EtOAc and washed with water (3×). The organics were concentrated and purified by silica gel chromatography (1-10% MeOH/DCM), the product was concentrated to give tert-butyl (S)-2-(3-allyl-4-fluorophenyl)-3-[3-(3-chloro-6-methyl-2-pyridyl)-2-oxo-2,3-dihydro-1H-imidazol-1-yl]-4-methyl-1,2,5-triaza-2,4,6,7-tetrahydro-5H-indene-5-carboxylate (135 mg) as an oil. m/z (ESI-pos) M+1=579.3

Step B: A solution of tert-butyl (S)-2-(3-allyl-4-fluorophenyl)-3-[3-(3-chloro-6-methyl-2-pyridyl)-2-oxo-2,3-dihydro-1H-imidazol-1-yl]-4-methyl-1,2,5-triaza-2,4,6,7-tetrahydro-5H-indene-5-carboxylate (135 mg, 233 μmol), allyltris (butyl) stannane (216 μL, 699 μmol), CataXCium A Pd G3 (34 mg, 46.6 μmol), and CsF (106 mg, 699 μmol) in 1.2 mL of dioxane was vacuum/backfilled with nitrogen and heated to 90° C. overnight. The solution was then diluted with filtered through celite, concentrated, and purified by silica gel chromatography chromatography (1-10% MeOH/DCM). This provided tert-butyl (S)-2-(3-allyl-4-fluorophenyl)-3-[3-(3-allyl-6-methyl-2-pyridyl)-2-oxo-2,3-dihydro-1H-imidazol-1-yl]-4-methyl-1,2,5-triaza-2,4,6,7-tetrahydro-5H-indene-5-carboxylate (91 mg) as an oil. m/z (ESI-pos) M+1=585.4

Step C: A solution of tert-butyl (S)-2-(3-allyl-4-fluorophenyl)-3-[3-(3-allyl-6-methyl-2-pyridyl)-2-oxo-2,3-dihydro-1H-imidazol-1-yl]-4-methyl-1,2,5-triaza-2,4,6,7-tetrahydro-5H-indene-5-carboxylate (91 mg), Hoveyda-Grubbs II (50.4 mg, 80.4 μmol), and acetic acid (4.6 μL, 80.4 μmol) in 35 mL of DCE was bubbled with nitrogen for 5 minutes and heated to 40° C. overnight. The mixture was then concentrated and purified by silica gel chromatography (1-10% MeOH/DCM), to give tert-butyl (S)-26-fluoro-8,17-dimethyl-30-oxo-2,3,7,11,14,16-hexaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (84 mg) as an oil. m/z (ESI-pos) M+1=557.4

Step D: A solution of tert-butyl (S)-26-fluoro-8,17-dimethyl-30-oxo-2,3,7,11,14,16-hexaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (81 mg, 146 μmol) and Pd/C 10% w/w (15.5 mg) in 1.5 mL of MeOH was sparged with hydrogen and then sealed and stirred for 1 hour. The solution was then filtered through celite, concentrated, and stirred in 0.5 mL of 4M HCl in dioxane for 1 hour. The solution was then filtered and taken forward as the crude(S)-26-fluoro-8,17-dimethyl-2,3,7,11,14,16-hexaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (70 mg) as an oil. m/z (ESI-pos) M+1=459.4

Step E: A solution of(S)-26-fluoro-8,17-dimethyl-2,3,7,11,14,16-hexaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (65 mg, 131 μmol), 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (70.2 mg, 171 μmol), HATU (74.9 mg, 197 μmol), and DIEA (68.6 μL, 394 μmol) in 0.2 mL of DMF was stirred for 1 hour. The solution was then taken up in EtOAc and washed with water (3×). The organics were concentrated and purified by silica gel chromatography (1-10% MeOH/DCM) to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8,17-dimethyl-2,3,7,11,14,16-hexaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (35 mg, 38.6 μmol) as a white solid. m/z (ESI-pos) M+1=852.6. ¹H NMR (400 MHz, chloroform-D) δ 11.22 (t, J=15.8 Hz, 1H), 8.00 (s, 1H), 7.56 (dd, J=16.8, 8.1 Hz, 2H), 7.49 (d, J=7.1 Hz, 1H), 7.45-7.36 (m, 1H), 7.15 (t, J=8.0 Hz, 2H), 6.74-6.38 (m, 2H), 6.27-6.06 (m, 1H), 5.87-5.68 (m, 1H), 4.54-4.35 (m, 1H), 3.83 (d, J=13.7 Hz, 2H), 3.58 (dd, J=24.1, 14.6 Hz, 1H), 3.36 (q, J=7.2 Hz, 1H), 3.17 (s, OH), 3.00 (d, J=12.4 Hz, 3H), 2.94 (s, 5H), 2.87 (d, J=0.8 Hz, 3H), 2.56-2.43 (m, 3H), 2.32 (d, J=17.0 Hz, 1H), 1.81-1.65 (m, 2H), 1.64-1.57 (m, 2H), 1.56-1.48 (m, 2H), 1.33 (d, J=3.0 Hz, 3H), 1.27 (d, J=3.7 Hz, 3H), 1.21 (dd, J=21.2, 6.0 Hz, 3H).

### Example 17—Synthesis of Compound 17

### Synthetic Scheme

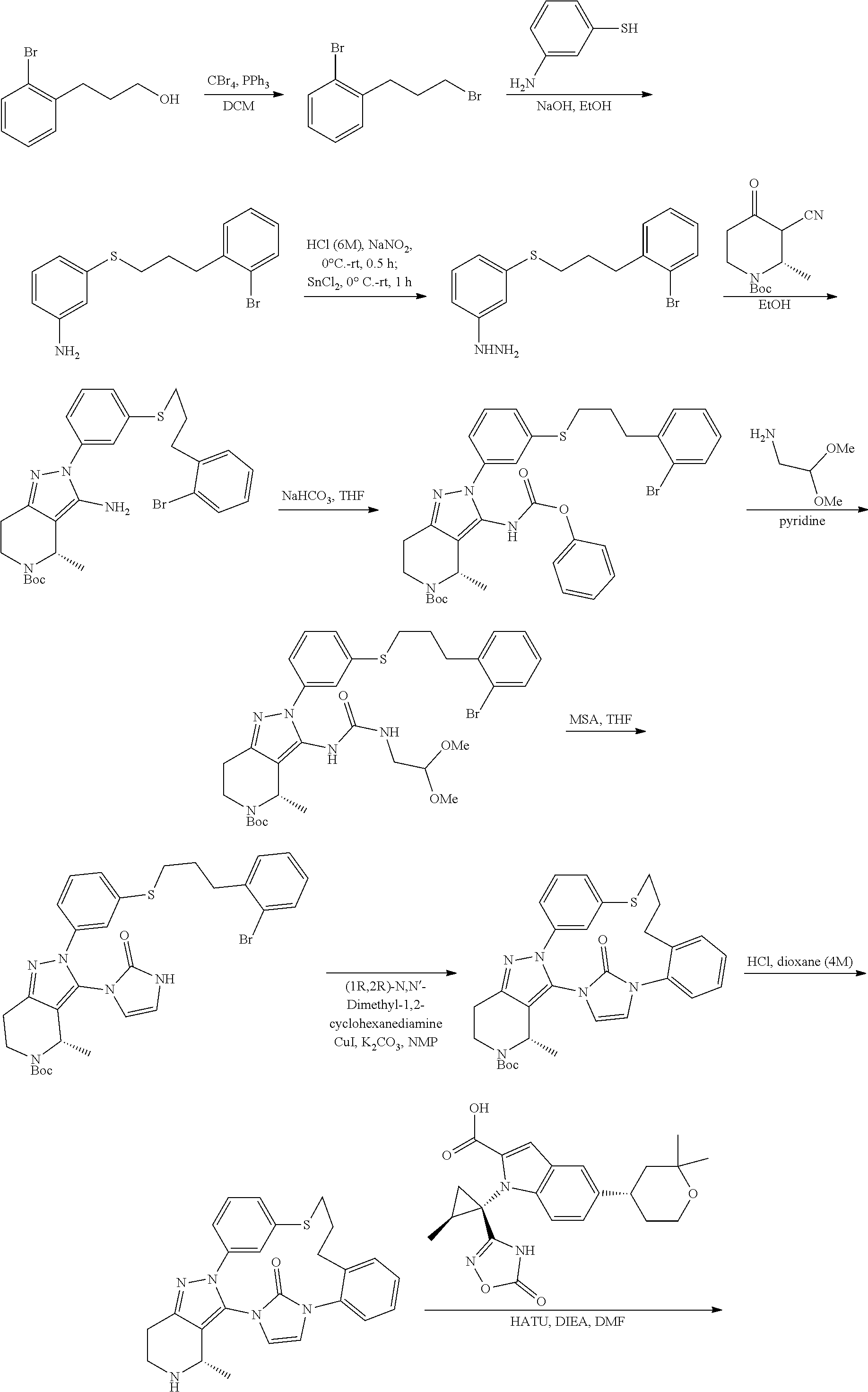

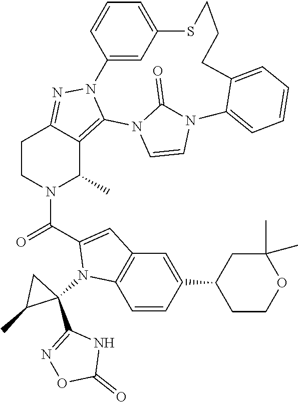

### Procedure for Synthesis of Compound 17

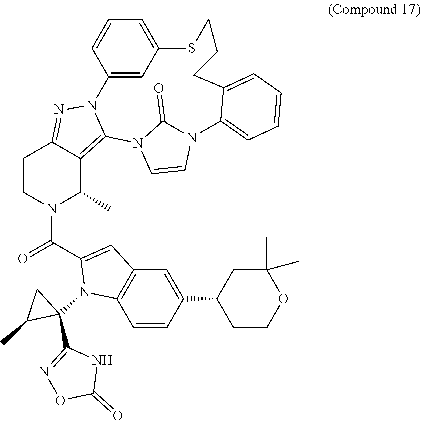

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-24-thia-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a mixture of 3-(o-bromophenyl)-1-propanol (5 g, 23.2 mmol) and perbromomethane (8.09 g, 24.4 mmol) in DCM (0.1 L) was added PPh₃ (6.4 g, 24.4 mmol) at 0° C. in batches, and then stirred at room temperature for 16 h. The reaction mixture was quenched with aqueous NaHCO₃ solution and extracted with DCM (2×). The combined organic layers were washed with brine, dried over Na₂SO₄, filtered and concentrated to give a residue, which was purified by column chromatography (PE/EtOAc=100/1) to give o-bromo (3-bromopropyl)benzene (5.1 g, yield 79%) as a colorless oil. ¹H NMR (400 MHz, CDCl3) δ 7.54 (d, J=8.0 Hz, 1H), 7.27-7.21 (m, 2H), 7.12-7.05 (m, 1H), 3.43 (t, J=6.8 Hz, 2H), 2.90 (t, J=7.6 Hz, 2H), 2.25-2.13 (m, 2H).

Step B: To a solution of m-aminobenzenethiol (2.3 g, 18.3 mmol) in NaOH aqueous solution (50 mL. 1M) and EtOH (100 mL) was added o-bromo (3-bromopropyl)benzene (5.1 g, 18.3 mmol) in EtOH (5 mL) dropwise at 0° C. and then stirred at room temperature for 2 h. The reaction mixture was diluted with water (200 mL), extracted with EtOAc (2×). The combined organic layers were washed with brine, dried over Na₂SO₄, filtered and concentrated to give a residue, which was purified by column chromatography (PE/EtOAc=3/1) to give m-[3-(o-bromophenyl)propylthio]aniline (5 g) as a colorless oil. m/z (ESI-pos) M+1=322.0/324.0

Step C: To a solution of m-[3-(o-bromophenyl)propylthio]aniline (4.5 g, 14 mmol) in aqueous HCl (100 mL, 6 M) was added sodium nitrite (1.06 g, 15.4 mmol) in water (5 mL) at 0° C. and the reaction mixture was stirred at room temperature for 0.5 h. SnCl₂ in con. HCl (10 mL) was added into the above reaction mixture at 0° C. and the reaction mixture was stirred at ambient temperature for 1 h. The reaction mixture was basified with NaOH solution (5 M) until pH 9 and the reaction mixture was extracted with EtOAc (2×). The combined organic layers were washed with brine, dried over Na₂SO₄, filtered and concentrated to give a residue, which was purified by column chromatography (PE/EtOAc=1/1)) to give {m-[3-(0-bromophenyl)propylthio]phenyl}hydrazine (0.7 g, yield 15%) as light yellow oil. m/z (ESI-pos) M+1=337.1

Step D: A mixture of {m-[3-(o-bromophenyl)propylthio]phenyl}hydrazine (0.7 g, 2.08 mmol) and tert-butyl(2S)-3-cyano-2-methyl-4-oxo-1-piperidinecarboxylate (544 mg, 2.28 mmol) in ethanol (20 mL) was stirred at 80° C. under N₂ atmosphere for 16 h. The reaction mixture was cooled to ambient temperature and then concentrated to give a residue, which was purified by column chromatography (PE/EtOAc=3/1) to give tert-butyl (S)-3-amino-2-{m-[3-(o-bromophenyl)propylthio]phenyl}-4-methyl-1,2,5-triaza-2,4,6,7-tetrahydro-5H-indene-5-carboxylate (550 mg, yield 48%) as a light yellow solid. m/z (ESI-pos) M+1=557.2

Step E: a mixture of tert-butyl (S)-3-amino-2-{m-[3-(o-bromophenyl)propylthio]phenyl}-4-methyl-1,2,5-triaza-2,4,6,7-tetrahydro-5H-indene-5-carboxylate (550 mg, 986 μmol) and sodium hydrogencarbonate (249 mg, 2.96 mmol) in tetrahydrofuran (10 mL) was added benzenyl chloroformate (232 mg, 1.48 mmol) at 0° C. under N₂ and the mixture was allowed to warm to room temperature and stirred for 4 h. The reaction mixture was then filtered and the filtrate was concentrated to give tert-butyl (S)-2-{m-[3-(o-bromophenyl)propylthio]phenyl}-4-methyl-3-[phenyl(oxycarbonylamino)]-1,2,5-triaza-2,4,6,7-tetrahydro-5H-indene-5-carboxylate (0.7 g) as a brown oil. m/z (ESI-pos) M+1=677.2

Step F: To a solution of tert-butyl (S)-2-{m-[3-(o-bromophenyl)propylthio]phenyl}-4-methyl-3-[phenyl(oxycarbonylamino)]-1,2,5-triaza-2,4,6,7-tetrahydro-5H-indene-5 carboxylate (0.7 g, 1.03 mmol) in pyridine (10 mL) was added 2,2-dimethoxyethylamine (326 mg, 3.1 mmol) at 0° C. under N₂ and the mixture was allowed to warm to ambient temperature and stirred for 4 h. The reaction was concentrated to give a residue which was purified by column chromatography (PE/EtOAc=1/1) to give tert-butyl (S)-2-{m-[3-(o-bromophenyl)propylthio]phenyl}-3-[3-(2,2-dimethoxyethyl)ureido]-4-methyl-1,2,5-triaza-2,4,6,7-tetrahydro-5H-indene-5-carboxylate (430 mg, yield 64%) as a light yellow solid. m/z (ESI-pos) M+1=688.2

Step G: To a solution of tert-butyl (S)-2-{m-[3-(o-bromophenyl)propylthio]phenyl}-3-[3-(2,2-dimethoxyethyl)ureido]-4-methyl-1,2,5-triaza-2,4,6,7-tetrahydro-5H-indene-5-carboxylate (460 mg, 668 μmol) in tetrahydrofuran (25 mL) was added methanesulfonic acid (64.2 mg, 668 μmol) at 0° C. under N₂ and the mixture was warmed to 60° C. and stirred for 2 h. The reaction was quenched with NaHCO₃ aqueous solution (50 mL) and extracted with EtOAc (2×). The combined organics were washed with brine, dried over Na₂SO₄, filtered and then concentrated under reduced pressure. The obtained residue was purified by column chromatography (PE/EtOAc=1/1) to give tert-butyl (S)-2-{m-[3-(o-bromophenyl)propylthio]phenyl}-4-methyl-3-(2-oxo-2,3-dihydro-1H-imidazol-1-yl)-1,2,5-triaza-2,4,6,7-tetrahydro-5H-indene-5-carboxylate (350 mg, yield 84%) as a light yellow solid. m/z (ESI-pos) M+1=624.1

Step H: To a mixture of tert-butyl (S)-2-{m-[3-(o-bromophenyl)propylthio]phenyl}-4-methyl-3-(2-oxo-2,3-dihydro-1H-imidazol-1-yl)-1,2,5-triaza-2,4,6,7-tetrahydro-5H-indene-5-carboxylate (0.3 g, 480 μmol), dipotassium carbonate (199 mg, 1.44 mmol) in N-methyl-2-pyrrolidone (30 mL) was added CuI (45.6 mg, 240 μmol) and (1R,2R)-1,2-bis(methylamino)cyclohexane (68.3 mg, 480 μmol) under N₂ and the mixture was stirred at 100° C. for 16 h. The reaction was quenched with water and extracted with EtOAc (2×). The combined organics were washed with brine, dried over Na₂SO₄, filtered and concentrated under reduced pressure. The obtained residue was purified by column chromatography (PE/EtOAc=1/1) to give tert-butyl (S)-8-methyl-30-oxo-24-thia-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (0.1 g) as a light yellow solid. m/z (ESI-pos) M+1=544.2; ¹H NMR (400 MHz, CDCl₃) δ 7.54-7.49 (m, 1H), 7.36 (s, 1H), 7.29-7.26 (m, 1H), 7.25-7.17 (m, 4H), 7.09-7.02 (m, 1H), 6.29 (s, 1H), 6.11 (s, 1H), 5.29 (s, 1H), 4.60-4.23 (m, 1H), 3.19-3.05 (m, 1H), 2.95 (t, J=7.2 Hz, 2H), 2.86 (t, J=7.2 Hz, 2H), 2.83-2.75 (m, 2H), 1.99-1.89 (m, 2H), 1.49 (s, 9H), 1.29-1.24 (m, 3H).

Step I: A solution of tert-butyl (S)-8-methyl-30-oxo-24-thia-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (0.1 g, 184 μmol) in HCl/1,4-dioxane (5 mL, 4 M) was stirred at room temperature for 1 h. The reaction mixture was concentrated under reduced pressure to give crude(S)-8-methyl-24-thia-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (85 mg) as a light yellow solid. m/z (ESI-pos) M+1=444.2

Step J: To a mixture of(S)-8-methyl-24-thia-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (85 mg, 192 μmol), 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (86.7 mg, 211 μmol) and DIEA (248 mg, 1.92 mmol) in DMF (10 mL) was added HATU (109 mg, 287 μmol) under N₂ and the mixture was stirred at room temperature for 16 h. The reaction was quenched with water and extracted with EtOAc (2×). The combined organics were washed with brine, dried over Na₂SO₄, filtered and concentrated under reduced pressure. The obtained residue was purified by Prep-HPLC [Waters MS-triggered Prep-LC with QDA detector, WELCH ultimate C18 21.2*250 mm 10 μm, eluted with 70-100% ACN in H₂O (0.1% FA), flow rate: 25 mL/min] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-24-thia-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (91.7 mg) as a white solid. m/z (ESI-pos) M+1=837.3; ¹H NMR (400 MHz, DMSO) δ 11.60 (s, 1H), 7.56-7.20 (m, 10.5H), 7.05-6.65 (m, 3.5H), 5.63 (s, 1H), 4.43 (s, 1H), 3.75-3.68 (m, 2H), 3.66-3.45 (m, 1H), 3.04-2.66 (m, 7H), 1.88-1.40 (m, 12H), 1.33-1.10 (m, 9H).

### Example 18—Synthesis of Compound 18

### Synthetic Scheme

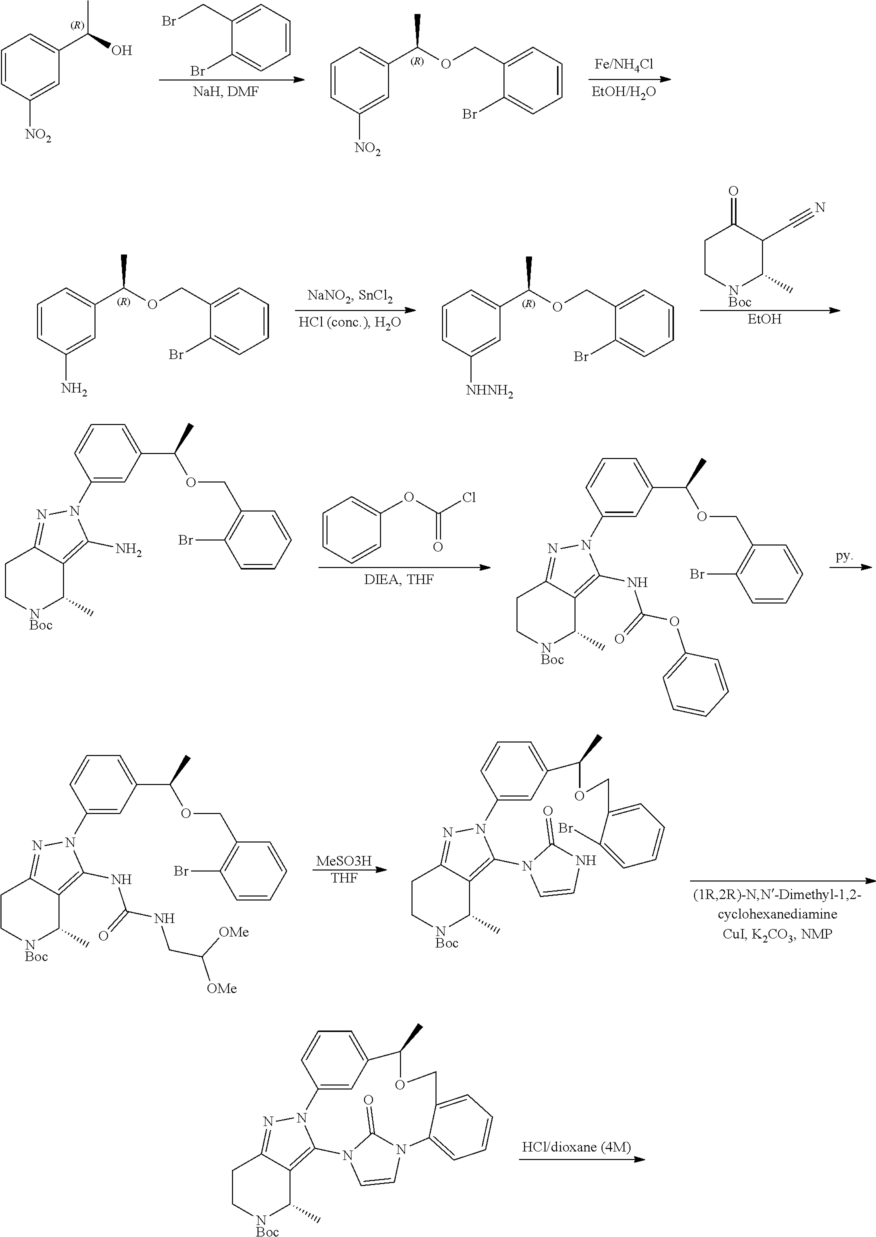

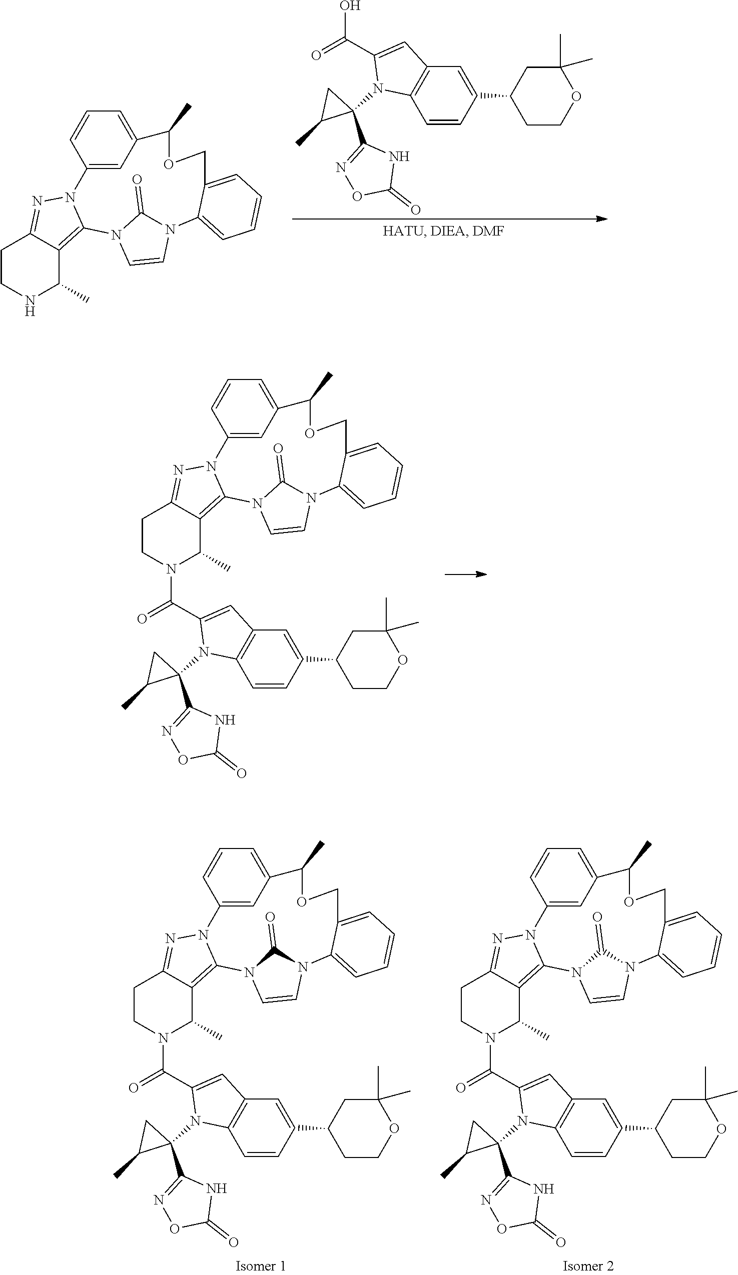

### Procedure for Synthesis of Compound 18

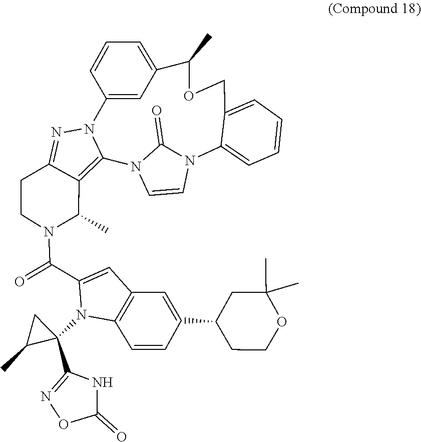

### (8S,23R)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8,23-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To the solution of (R)-1-(3-nitrophenyl) ethan-1-ol (2 g, 12 mmol) in DMF (30 mL) was added Sodium hydride (60% w/w, 957 mg, 23.9 mmol) at 0° C. under N₂. The reaction mixture was stirred at 0° C. for 30 min. Then 1-bromo-2-(bromomethyl)benzene (2.99 g, 12 mmol) was added to the mixture and the mixture was allowed to warm to ambient temperature where it stirred for 2 h. The reaction mixture was quenched with aq. NH₄Cl, diluted with ethyl acetate, washed with water (2×) and the aqueous layer was extracted with ethyl acetate (2×). The combined EtOAc layers were washed with brine, dried over Na₂SO₄ filtered and concentrated in vacuo. The crude product was purified by column chromatography (0-20% EtOAc in PE). to give (R)-1-bromo-2-((1-(3-nitrophenyl) ethoxy)methyl)benzene (4 g, 10.7 mmol) as a yellow oil. 1HNMR (400 MHz, CDCl₃) δ 8.16 (s, 1H), 8.12 (d, J=8.4 Hz, 1H), 7.60 (d, J=7.6 Hz, 1H), 7.55-7.46 (m, 2H), 7.31-7.23 (m, 2H), 7.11-7.07 (m, 1H), 4.61 (s, 2H), 3.76 (t, J=6.8 Hz, 2H), 3.11 (t, J=6.8 Hz, 2H).

Step B: To the solution of (R)-1-bromo-2-((1-(3-nitrophenyl) ethoxy)methyl)benzene (4 g, 10.7 mmol) in EtOH (0.1 L) was added H₂O (25 mL), iron (2.99 g, 53.5 mmol) and ammonium chloride (2.86 g, 53.5 mmol). The reaction mixture was stirred at 70° C. for 4 h. The mixture was cooled to ambient temperature and diluted with H₂O, extracted with EtOAc (2×). The combined EtOAc layers were washed with brine, dried over Na₂SO₄ filtered and concentrated in vacuo. The crude product was purified by column chromatography (0-20% EtOAc in PE) to give (R)-3-(1-((2-bromobenzyl)oxy)ethyl)aniline (3 g, 8.82 mmol) as a yellow oil. m/z (ESI-pos) M+1=306.1/308.1.

Step C: To a solution of (R)-3-(1-((2-bromobenzyl)oxy)ethyl)aniline (3 g, 8.82 mmol) in conc. HCl (25 mL) and H₂O (25 mL) at 0° C. was added NaNO₂ (669 mg, 9.7 mmol) in H₂O (5 mL) dropwise. After the addition, the mixture was stirred at 0° C. for 45 mins. Then a SnCl₂ (3.68 g, 19.4 mmol) solution in conc. HCl (20 mL) and H₂O (20 mL) was added dropwise. The mixture was stirred at ambient temperature for another 1 hour and then quenched with aq. NaOH (10N, 55 mL) to adjust pH to about 9. Then the mixture was extracted with EtOAc (3×), the combined organic layers were dried over anhydrous Na₂SO₄, filtered and concentrated under vacuum. The crude product was purified by column chromatography (0-100% EtOAc in PE) to give (R)-(3-(1-((2-bromobenzyl)oxy)ethyl)phenyl)hydrazine (1.5 g, 4.2 mmol) as a yellow solid. m/z (ESI-pos) M+1=321.1/323.1

Step D: To a solution of (R)-(3-(1-((2-bromobenzyl)oxy)ethyl)phenyl)hydrazine (1.5 g, 4.2 mmol) in EtOH (30 mL) was added tert-butyl(2S)-3-cyano-2-methyl-4-oxo-1-piperidinecarboxylate (1.0 g, 4.2 mmol) and the mixture was stirred at 80° C. for 16 hours. The mixture was concentrated under reduced pressure to get the crude product, which was purified by column chromatography (0-33% EtOAc in PE) to give tert-butyl (S)-3-amino-2-(3-((R)-1-((2-bromobenzyl)oxy)ethyl)phenyl)-4-methyl-2,4,6,7-tetrahydro-5H-pyrazolo[4,3-c]pyridine-5-carboxylate (1.7 g, 2.83 mmol) as a yellow solid. m/z (ESI-pos) M+1=541.1/543.2.

Step E: To a solution of tert-butyl (S)-3-amino-2-(3-((R)-1-((2-bromobenzyl)oxy)ethyl)phenyl)-4-methyl-2,4,6,7-tetrahydro-5H-pyrazolo[4,3-c]pyridine-5-carboxylate (1.7 g, 2.83 mmol) in tetrahydrofuran (40 mL) at 0° C. was added DIEA (1.1 g, 8.48 mmol) and benzenyl chloroformate (885 mg, 5.65 mmol). The mixture was stirred at ambient temperature for 2 hours and then concentrated to get crude tert-butyl (S)-2-(3-((R)-1-((2-bromobenzyl)oxy)ethyl)phenyl)-4-methyl-3-((phenoxycarbonyl)amino)-2,4,6,7-tetrahydro-5H-pyrazolo[4,3-c]pyridine-5-carboxylate (2.65 g) as a yellow solid, which was used directly in next step. m/z (ESI-pos) M+1=661.2/663.2.

Step F: To a solution of crude tert-butyl (S)-2-(3-((R)-1-((2-bromobenzyl)oxy)ethyl)phenyl)-4-methyl-3-((phenoxycarbonyl)amino)-2,4,6,7-tetrahydro-5H-pyrazolo[4,3-c]pyridine-5-carboxylate (2.65 g, 2.8 mmol) in pyridine (20 mL) was added 2,2-dimethoxyethylamine (884 mg, 8.41 mmol). The mixture was stirred at ambient temperature for 2 hours and then concentrated under vacuum. The obtained residue was purified by column chromatography (0-100% EtOAc in PE) to give tert-butyl (S)-2-(3-((R)-1-((2-bromobenzyl)oxy)ethyl)phenyl)-3-(3-(2,2-dimethoxyethyl)ureido)-4-methyl-2,4,6,7-tetrahydro-5H-pyrazolo[4,3-c]pyridine-5-carboxylate (1.55 g) as a white solid. m/z (ESI-pos) M+1=672.2/674.2

Step G: To a solution of tert-butyl (S)-2-(3-((R)-1-((2-bromobenzyl)oxy)ethyl)phenyl)-3-(3-(2,2-dimethoxyethyl)ureido)-4-methyl-2,4,6,7-tetrahydro-5H-pyrazolo[4,3-c]pyridine-5-carboxylate (1.55 g, 2.07 mmol) in tetrahydrofuran (20 mL) was added methanesulfonic acid (239 mg, 2.49 mmol). The mixture was stirred at 60° C. for 1 hour and then cooled to ambient temperature. The reaction mixture was diluted with H₂O, extracted with EtOAc (2×). The combined organic layers were dried over anhydrous Na₂SO₄, filtered and concentrated. The crude product was purified by column chromatography (0-100% EtOAc in PE) to give tert-butyl (S)-2-(3-((R)-1-((2-bromobenzyl)oxy)ethyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (1.3 g) as a white solid. m/z (ESI-pos) M+1=608.2/610.2

Step H: To a solution of tert-butyl (S)-2-(3-((R)-1-((2-bromobenzyl)oxy)ethyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (1.3 g, 1.92 mmol) in NMP (100 mL) was added copper iodide (183 mg, 961 μmol), (1R,2R)—N,N′-Dimethyl-1,2 cyclohexane diamine (273 mg, 1.92 mmol) and K₂CO₃ (797 mg, 5.77 mmol). The mixture was stirred at 100° C. for 16 hours and then cooled to ambient temperature, diluted with H₂O and extracted with EtOAc (2×). The combined EtOAc layers were washed with aqueous LiCl (2×). Dried over Na₂SO₄, filtered and concentrated in vacuo. The crude product was purified by column chromatography (0-50% EtOAc in PE) to give tert-butyl (8S,23R)-8,23-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (400 mg) as a white solid. m/z (ESI-pos) M+1=528.3

Step I: A solution of tert-butyl (8S,23R)-8,23-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (180 mg, 307 μmol) in HCl/1,4-dioxane (4M, 10 mL) was stirred at 20° C. for 1 hour. The mixture was directly concentrated under reduced pressure to give crude (8S,23R)-8,23-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (144 mg) as a white solid. m/z (ESI-pos) M+1=428.2.

Step J: To a solution of (8S,23R)-8,23-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (144 mg, 303 μmol) in dimethylformamide (10 mL) was added 5-[(S)-2,2-dimethyltetrahydro-4H-pyran-4-yl]-1-[(2S)-2-methyl-1-(5-oxo-4,5 dihydro-1,2,4-oxadiazol-3-yl)cyclopropyl]-2-indolecarboxylic acid (125 mg, 303 μmol) and DIEA (157 mg, 1.21 mmol). The mixture was stirred at 20° C. for 5 mins, then HATU (173 mg, 455 μmol) was added into the mixture and stirred at 20° C. for 16 hours. H₂O was added and the mixture was extracted with EtOAc (2×). The combined EtOAc layers were washed with brine, then dried over Na₂SO₄, filtered and concentrated in vacuo. The crude residue was purified by column chromatography (0-5% MeOH in DCM) to give (8S,23R)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8,23-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (194 mg), which was further purified by prep-HPLC [Waters MS-triggered Prep-LC with QDA detector, WELCH Xtimate C18 21.2*250 mm 10 μm, eluted with 70-100% ACN in H₂O (0.1% FA)] to give:

Isomer 1 (11.4 mg) as a white solid. m/z (ESI-pos) M+1=821.3. 1H NMR (400 MHz, DMSO) δ 11.54 (s, 1H), 7.63-7.61 (m, 1H), 7.52-7.44 (m, 6H), 7.41 (d, J=8.4 Hz, 1H), 7.31-7.30 (m, 2H), 7.25-7.22 (m, 1H), 6.87 (s, 1H), 6.57 (d, J=2.4 Hz, 1H), 6.39 (d, J=2.8 Hz, 1H), 5.66 (s, 1H), 4.65-4.60 (m, 1H), 4.42 (s, 1H), 4.12 (s, 2H), 3.73-3.71 (m, 2H), 3.57 (s, 1H), 3.01-2.99 (m, 2H), 2.89-2.85 (m, 1H), 1.81-1.69 (m, 4H), 1.65-1.50 (m, 6H), 1.28-1.25 (m, 7H), 1.19 (s, 5H).

Isomer 2 (76.8 mg) as a white solid. m/z (ESI-pos) M+1=821.3; 1H NMR (400 MHz, DMSO) δ 11.57 (s, 1H), 7.72-7.70 (m, 1H), 7.54 (s, 1H), 7.46-7.37 (m, 6H), 7.27-7.21 (m, 2H), 7.01-6.88 (m, 3H), 6.73 (s, 1H), 5.67 (s, 1H), 4.52-4.47 (m, 2H), 4.24 (d, J=9.6 Hz, 1H), 3.90 (d, J=9.6 Hz, 1H), 3.74-3.72 (m, 2H), 3.56 (s, 1H), 3.04-3.00 (m, 2H), 2.94-2.88 (m, 1H), 1.74-1.54 (m, 7H), 1.51-1.47 (m, 3H), 1.35 (d, J=6.4 Hz, 3H), 1.28 (s, 3H), 1.20 (s, 6H).

(The absolute configuration was unknown, the configuration of two atropisomers were assigned arbitrarily).

### Example 19—Synthesis of Compound 19

### Procedure for Synthesis of Compound 19

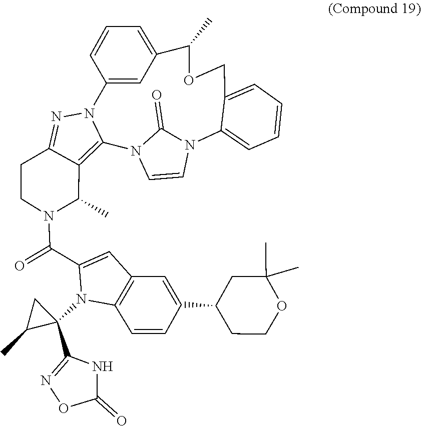

### (8S,23S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8,23-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 19 was prepared following an analogous procedure to that used for Compound 18 except that (R)-1-(3-nitrophenyl) ethan-1-ol was replaced with(S)-1-(3-nitrophenyl) ethan-1-ol in Step A. The title compound was purified by prep-HPLC [Instrument: Waters Prep-LC with UV detector; Waters Luna C18 10 μm 21.2*250 mm; Gradient: 40-80% ACN in H₂O (0.1% FA) in 18 min; Flow rate: 20 mL/min] to give Isomer 1 (35.3 mg) and Isomer 2 (20.7 mg) as white solids. (The absolute configuration was unknown, the configuration of two atropisomers were assigned arbitrarily).

Isomer 1: m/z (ESI-pos) M+1=821.3; ¹H NMR (400 MHz, DMSO) δ 11.66 (s, 1H), 7.61 (d, J=4.8 Hz, 1H), 7.54-7.34 (m, 8H), 7.29 (d, J=6.8 Hz, 1H), 7.22 (d, J=8.8 Hz, 1H), 6.82 (s, 1H), 6.45 (d, J=54.8 Hz, 2H), 5.32 (s, 0.5H), 4.61 (d, J=6.4 Hz, 1H), 4.55-4.21 (m, 0.5H), 4.09 (q, J=8.8 Hz, 2H), 3.73-3.58 (m, 3H), 2.94 (d, J=39.2 Hz, 3H), 1.79-1.49 (m, 10H), 1.33-1.24 (m, 8H), 1.19 (s, 5H).

Isomer 2: m/z (ESI-pos) M+1=821.3; ¹H NMR (400 MHz, DMSO) δ 11.56 (s, 1H), 7.72 (d, J=8.0 Hz, 1H), 7.57-7.32 (m, 7H), 7.26-7.17 (m, 2H), 6.96 (s, 2H), 6.84 (s, 1H), 6.73 (s, 1H), 5.61 (s, 1H), 4.53-4.38 (m, 2H), 4.17 (d, J=10.0 Hz, 1H), 3.89 (d, J=10.0 Hz, 1H), 3.71 (d, J=7.6 Hz, 2H), 3.58 (s, 1H), 3.05-2.82 (m, 3H), 1.81-1.44 (m, 10H), 1.36 (d, J=6.4 Hz, 3H), 1.26 (d, J=8.8 Hz, 4H), 1.18 (s, 5H).

### Example 20—Synthesis of Compound 20

### Synthetic Scheme

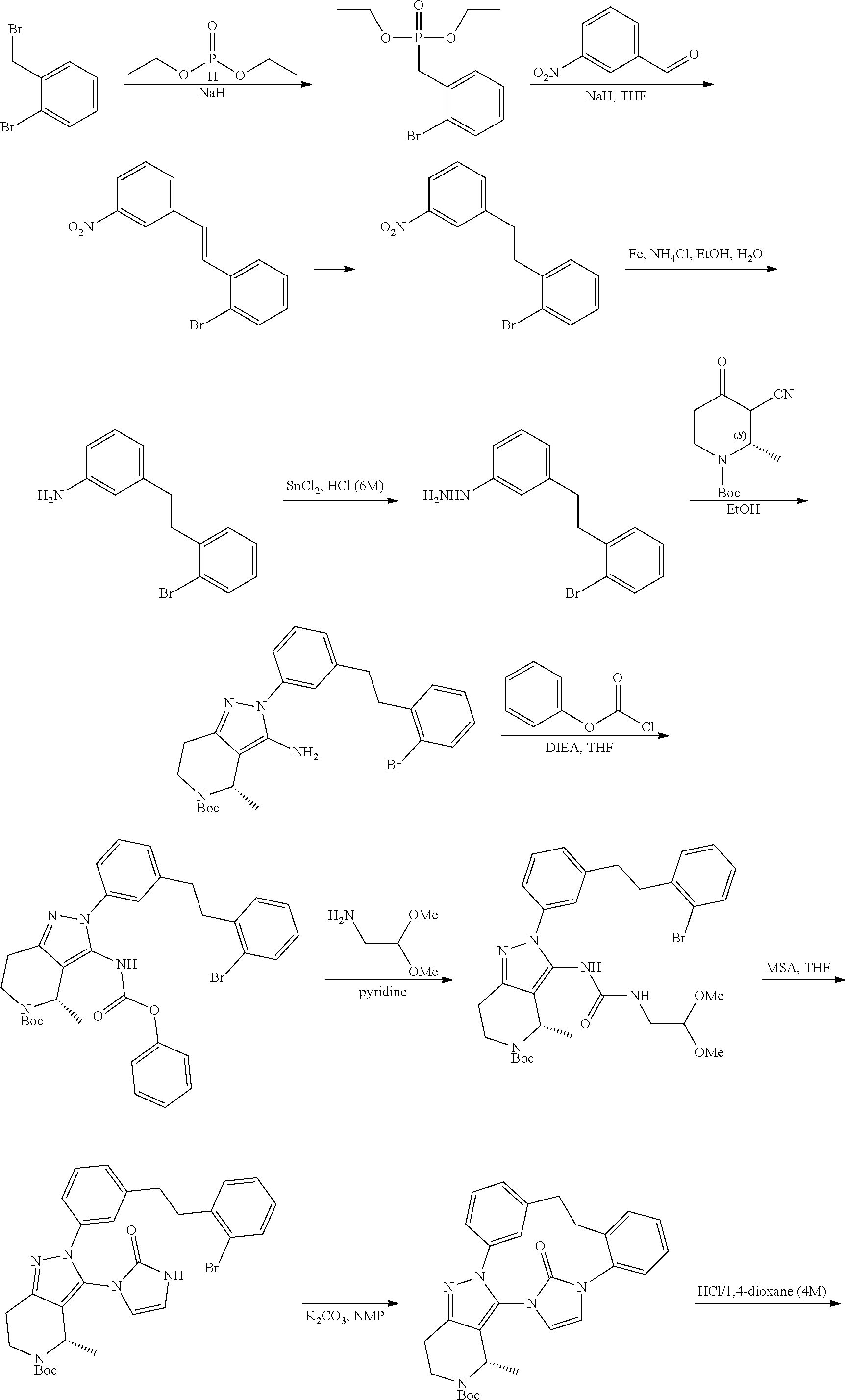

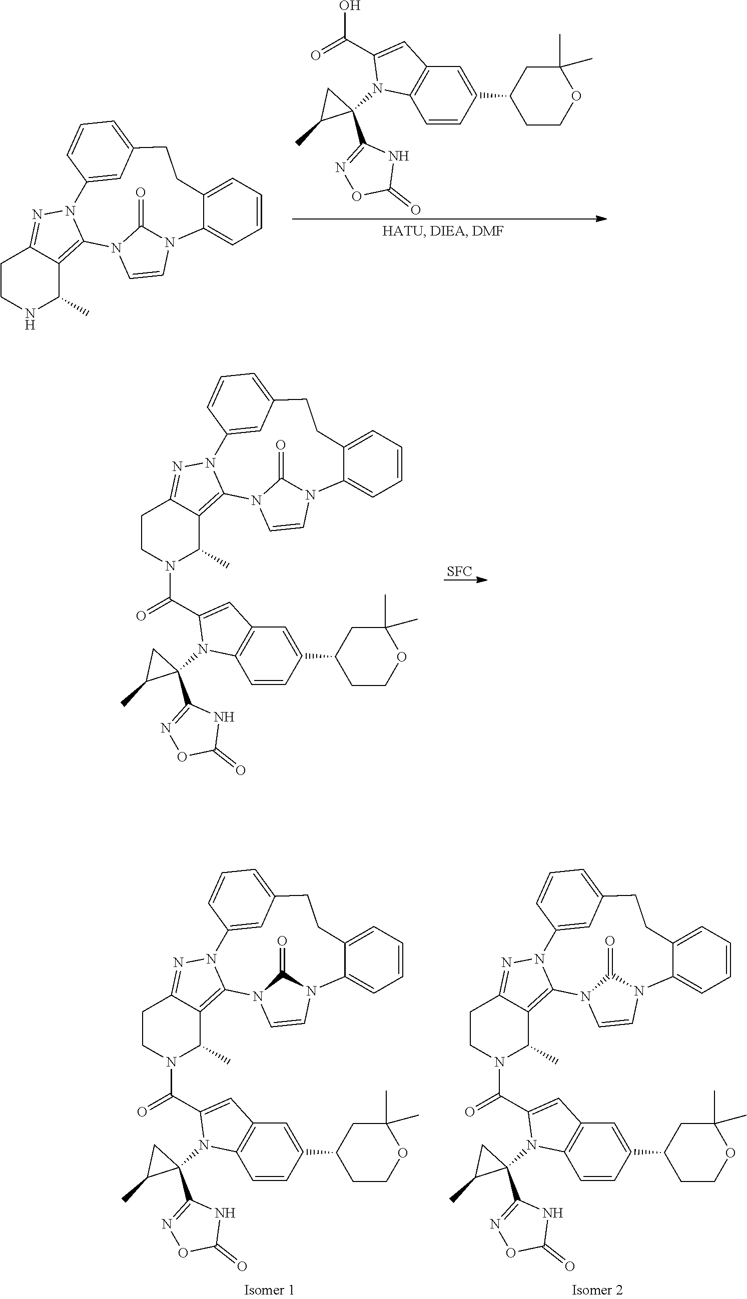

### Procedure for Synthesis of Compound 20

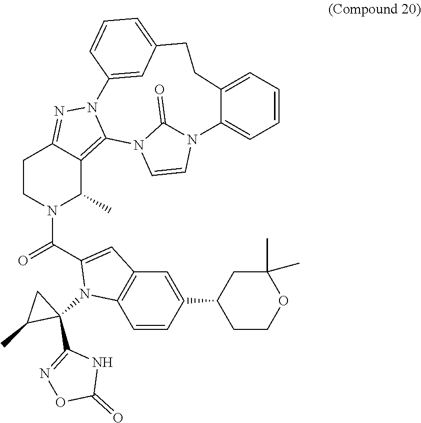

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaen-28-one

Step A: To a suspension of sodium hydride (576 mg, 24 mmol) in dimethylformamide (15.8 mL) under N₂ at 0° C., was added diethyl phosphonate (2.76 g, 20 mmol). The mixture was stirred at 0° C. for 30 min, then o-bromo(bromomethyl)benzene (5 g, 20 mmol) was added to the mixture dropwise. The resulting mixture was stirred at 0° C. for 3 h. The reaction was quenched with water and extracted with ethyl acetate (3×). The combined organic layers were washed with brine solution, dried over anhydrous Na₂SO₄, filtered and evaporated under reduced pressure to give the crude compound, which was purified by column chromatography (0-5% EtOAc in PE) to give diethyl [(o-bromophenyl)methyl]phosphonate (5 g, 13 mmol) as a white solid. m/z (ESI-pos) M+1=307.1/309.1

Step B: To a solution of diethyl [(o-bromophenyl)methyl]phosphonate (5 g, 13 mmol) in tetrahydrofuran (10 mL) was added sodium hydride (938 mg, 39.1 mmol) at −10° C. under N₂. Then a THF (10 mL) solution of m-nitrobenzaldehyde (1.38 g, 9.12 mmol) was added into the mixture dropwise over 2 mins. The mixture was allowed to warm to ambient temperature and stirred for 3 h. The reaction was quenched by pouring the mixture into ice water, extracted with ethyl acetate (2×). The combined organic layers were washed with brine solution and dried over anhydrous Na₂SO4. Filtered and the filtrate was evaporated under reduced pressure. The crude compound was purified by column chromatography (0-5% EtOAc in PE) to give (E)-2-bromo-5′-nitrostilbene (3.1 g, 10.2 mmol) as a yellow solid. ¹H NMR (400 MHz, CDCl₃) δ 8.43-8.33 (m, 1H), 8.13 (dd, J=8.0, 1.6 Hz, 1H), 7.84 (t, J=8.0 Hz, 1H), 7.70-7.51 (m, 4H), 7.35 (t, J=8.0 Hz, 1H), 7.18 (td, J=7.2, 1.6 Hz, 1H), 7.07 (d, J=16.4 Hz, 1H).

Step C: To a solution of (E)-2-bromo-5′-nitrostilbene (3.5 g, 11.5 mmol) in tetrahydrofuran (87.5 mL) was added tosylhydrazine (10.7 g, 57.5 mmol) and sodium acetate (4.72 g, 57.5 mmol). The mixture was stirred at 90° C. for 5 h and the mixture was cooled to ambient temperature, diluted with ethyl acetate, washed with water (2×) and the aqueous layer was extracted with ethyl acetate (2×). The combined organic layers were washed with brine solution and dried over anhydrous Na₂SO₄. Filtered and the filtrate was evaporated under reduced pressure to give the crude compound. The crude compound was purified by column chromatography (0-50% EtOAc in PE) to give o-bromo[2-(m-nitrophenyl)ethyl]benzene (2.8 g, 9.15 mmol) as a white solid. ¹H NMR (400 MHz, CDCl₃) δ 8.10-8.04 (m, 2H), 7.56 (dd, J=8.0, 1.2 Hz, 1H), 7.50 (d, J=7.6 Hz, 1H), 7.46-7.41 (m, 1H), 7.23-7.19 (m, 1H), 7.15-7.04 (m, 2H), 3.19-2.92 (m, 4H).

Step D: To a solution of o-bromo[2-(m-nitrophenyl)ethyl]benzene (2.8 g, 9.15 mmol) in ethanol (93.3 mL) and water (9.33 mL) was added iron powder (2.55 g, 45.7 mmol) and ammonium chloride (2.45 g, 45.7 mmol). The reaction mixture was stirred at 70° C. for 2 h. The reaction mixture was cooled to ambient temperature and quenched with H₂O, extracted with ethyl acetate (2×). The combined EtOAc layers were washed with brine, dried over Na₂SO₄ and concentrated. The crude product was purified by column chromatography (0-5% DCM in MeOH). to give m-[2-(o-bromophenyl)ethyl]aniline (2.3 g, 8.33 mmol) as a yellow oil. m/z (ESI-pos) M+1=276.1/278.1

Step E: To a solution of m-[2-(o-bromophenyl)ethyl]aniline (2.3 g, 8.33 mmol) in 6M aq. HCl at 0° C. was added a solution of sodium nitrite (575 mg, 8.33 mmol) in water (5 mL) dropwise. The mixture was then stirred at 0° C. for 1 h, a solution of SnCl₂ (3.16 g, 16.7 mmol) in conc. hydrogen chloride (40 mL) was then added dropwise to the reaction mixture and then the mixture was allowed to warm to ambient temperature and stirred for 2 h. The pH was adjusted to about 14 with 4N aq. NaOH and the mixture extracted with ethyl acetate (3×). The combined organic layers were washed with brine solution and dried over anhydrous Na₂SO₄. Filtered and the filtrate was evaporated under reduced pressure. The residue was purified by column chromatography (0-80% PE in EtOAc) to give {m-[2-(0-bromophenyl)ethyl]phenyl}hydrazine (1.8 g, 6.18 mmol) as a yellow oil. m/z (ESI-pos) M+1=291.1/293.1

Step F: To a solution of {m-[2-(o-bromophenyl)ethyl]phenyl}hydrazine (1.8 g, 6.18 mmol) in ethanol (48 mL) at ambient temperature was added tert-butyl(2S)-3-cyano-2-methyl-4-oxo-1-piperidinecarboxylate (1.47 g, 6.18 mmol) and the mixture was stirred at 80° C. for 16 hours. The mixture was concentrated under reduced pressure to get crude product, which was purified by column chromatography (0-60% EtOAc in PE) to give tert-butyl (S)-3-amino-2-{m-[2-(o-bromophenyl)ethyl]phenyl}-4-methyl-1,2,5-triaza-2,4,6,7-tetrahydro-5H-indene-5-carboxylate (2.5 g, 4.89 mmol) as a yellow solid. m/z (ESI-pos) M+1=511.2/513.2

Step G: To a solution of tert-butyl (S)-3-amino-2-{m-[2-(o-bromophenyl)ethyl]phenyl}-4-methyl-1,2,5-triaza-2,4,6,7-tetrahydro-5H-indene-5-carboxylate (2.5 g, 4.89 mmol) in tetrahydrofuran (50 mL) at 0° C. was added N-ethyldiisopropylamine (1.9 g, 14.7 mmol) and benzenyl chloroformate (1.53 g, 9.78 mmol). The mixture was stirred at ambient temperature for 2 hours and then concentrated to get crude tert-butyl (S)-2-{m-[2-(o-bromophenyl)ethyl]phenyl}-4-methyl-3-[phenyl(oxycarbonylamino)]-1,2,5-triaza-2,4,6,7-tetrahydro-5H-indene-5-carboxylate (3 g, 3.8 mmol) as a yellow solid, which was used directly in the next step. m/z (ESI-pos) M+1=631.2/633.2

Step H: To a solution of crude tert-butyl (S)-2-{m-[2-(o-bromophenyl)ethyl]phenyl}-4-methyl-3-[phenyl(oxycarbonylamino)]-1,2,5-triaza-2,4,6,7-tetrahydro-5H-indene-5-carboxylate (3 g, 3.8 mmol) in pyridine (40 mL) was added 2,2-dimethoxyethylamine (2.5 g, 23.8 mmol). The mixture was stirred at ambient temperature for 2 hours and then concentrated under vacuum. The obtained residue was purified by column chromatography (0-80% EA in PE) to give tert-butyl (S)-2-{m-[2-(o-bromophenyl)ethyl]phenyl}-3-[3-(2,2-dimethoxyethyl)ureido]-4-methyl-1,2,5-triaza-2,4,6,7-tetrahydro-5H-indene-5-carboxylate (2 g, 3.11 mmol) as a white solid. m/z (ESI-pos) M+1=642.2/644.2

Step I: tert-butyl (S)-2-{m-[2-(o-bromophenyl)ethyl]phenyl}-3-[3-(2,2-dimethoxyethyl)ureido]-4-methyl-1,2,5-triaza-2,4,6,7-tetrahydro-5H-indene-5-carboxylate (2 g, 3.11 mmol) in tetrahydrofuran (40 mL) was added methanesulfonic acid (299 mg, 3.11 mmol). The mixture was stirred at 60° C. for 1 hour and then cooled to ambient temperature. The reaction mixture was diluted with H₂O, extracted with EtOAc (2×). The combined organic layers were dried over anhydrous Na₂SO₄, filtered and concentrated under vacuum to get crude product, which was purified by column chromatography (0-80% EtOAc in PE) to give tert-butyl(S)-2-{m-[2-(o-bromophenyl)ethyl]phenyl}-4-methyl-3-(2-oxo-2,3-dihydro-1H-imidazol-1-yl)-1,2,5-triaza-2,4,6,7-tetrahydro-5H-indene-5-carboxylate (1.8 g, 3.11 mmol) as a white solid. m/z (ESI-pos) M+1=578.2/580.2

Step J: To a solution of tert-butyl (S)-2-{m-[2-(o-bromophenyl)ethyl]phenyl}-4-methyl-3-(2-oxo-2,3-dihydro-1H-imidazol-1-yl)-1,2,5-triaza-2,4,6,7-tetrahydro-5H-indene-5-carboxylate (0.9 g, 1.56 mmol) in N-methyl-2-pyrrolidone (36 mL) was added copper iodide (296 mg, 1.56 mmol), (1R,2R)-1,2-bis(methylamino)cyclohexane (443 mg, 3.11 mmol) and dipotassium carbonate (415 g, 3 mol). The mixture was stirred at 100° C. for 16 hours. The reaction mixture was cooled to ambient temperature and diluted with ethyl acetate, washed with water (2×) and the aqueous layer was extracted with ethyl acetate (2×). The combined organic layers were washed with aq. LiCl solution and dried over anhydrous Na₂SO₄. Filtered and the filtrate was evaporated under reduced pressure. The obtained residue was purified by column chromatography (0-80% EtOAc in PE) to give tert-butyl (S)-8-methyl-28-oxo-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaene-7-carboxylate (220 mg, 442 μmol) as yellow solid. m/z (ESI-pos) M+1=498.2

Step K: A solution of tert-butyl (S)-8-methyl-28-oxo-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaene-7-carboxylate (210 mg, 422 μmol) in HCl/1,4-dioxane (4M, 2 mL) was stirred at ambient temperature for 1 hour. Then the mixture was directly concentrated under vacuum to give crude (S)-8-methyl-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaen-28-one hydrogen chloride (180 mg, 453 μmol) as a white solid. m/z (ESI-pos) M+1=398.2

Step L To a solution of(S)-8-methyl-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaen-28-one hydrogen chloride (180 mg, 453 μmol) in dimethylformamide (6 mL) was added 5-[(S)-2,2-dimethyltetrahydro-4H-pyran-4-yl]-1-[(2S)-2-methyl-1-(5-oxo-4,5-dihydro-1,2,4-oxadiazol-3-yl)cyclopropyl]-2-indolecarboxylic acid (186 mg, 453 μmol) and N-ethyldiisopropylamine (176 mg, 1.36 mmol). The mixture was stirred for 5 mins, then HATU (258 mg, 679 μmol) was added into and the mixture was stirred at ambient temperature for 16 hours. H₂O was added to quench the reaction and the mixture was extracted with EtOAc (2×). The combined EtOAc layers were washed with brine, dried over Na₂SO₄, filtered and concentrated in vacuo to get crude product, which was purified by column chromatography (0-5% MeOH in DCM) to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaen-28-one (80 mg, 101 μmol) as a yellow solid, which was separated by SFC [Column:: Daicel CHIRALCEL IC, 250 mm×30 mm I.D., 10 μm: Hex/EtOH [0.2% TFA]=50/50] to get isomer 1 (12.2 mg) and isomer 2 (12.0 mg). (The absolute configuration was unknown, the configuration of two atropisomers were assigned arbitrarily) Isomer 1: m/z (ESI-pos) M+1=791.3; 1H NMR (400 MHz, DMSO) δ 11.49 (s, 1H), 7.49 (d, J=7.6 Hz, 2H), 7.44-7.30 (m, 2H), 7.26-7.10 (m, 6H), 6.89 (dd, J=14.0, 6.8 Hz, 3H), 6.53 (s, 1H), 5.63 (s, 1H), 4.40 (s, 1H), 3.75-3.68 (m, 2H), 3.59 (s, 1H), 3.34 (dd, J=39.2, 13.6 Hz, 2H), 2.99 (s, 2H), 2.90 (d, J=7.2 Hz, 2H), 2.82-2.72 (m, 1H), 1.77-1.48 (m, 9H), 1.22 (dd, J=35.6, 10.8 Hz, 10H).

Isomer 2: m/z (ESI-pos) M+1=791.3; 1H NMR (400 MHz, DMSO) δ 11.58 (s, 1H), 7.55-7.46 (m, 2H), 7.42 (d, J=8.8 Hz, 1H), 7.37-7.32 (m, 1H), 7.26-7.12 (m, 7H), 6.88 (d, J=7.6 Hz, 2H), 6.49 (s, 1H), 5.74 (s, 1H), 4.45 (s, 1H), 3.73 (dd, J=12.0, 19.2 Hz, 2H), 3.56 (s, 1H), 3.44-3.22 (m, 2H), 3.01 (d, J=3.6 Hz, 2H), 2.94-2.83 (m, 2H), 2.81-2.71 (m, 1H), 1.80-1.46 (m, 9H), 1.36-1.11 (m, 10H).

### Example 21—Synthesis of Compound 21

### Synthetic Scheme

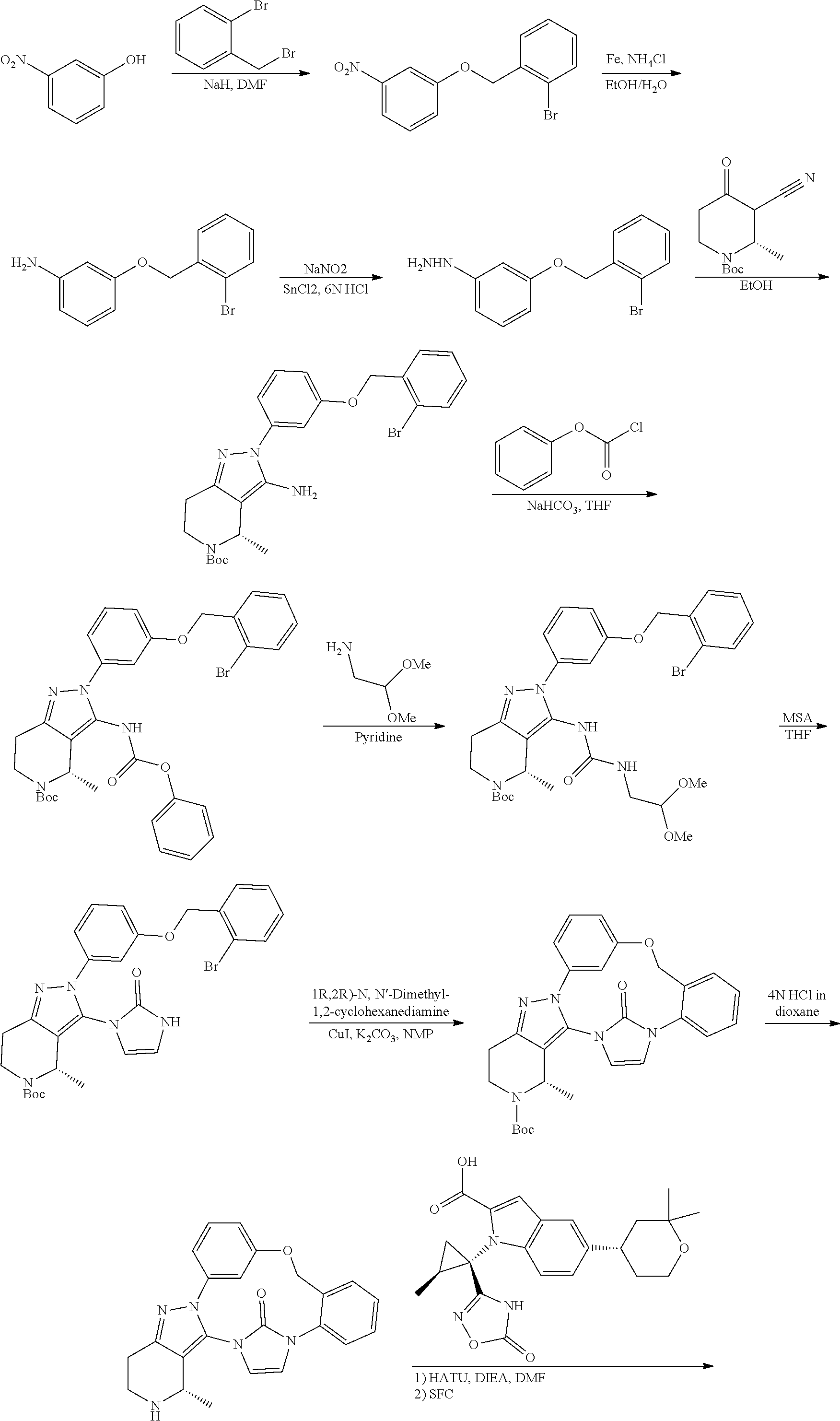

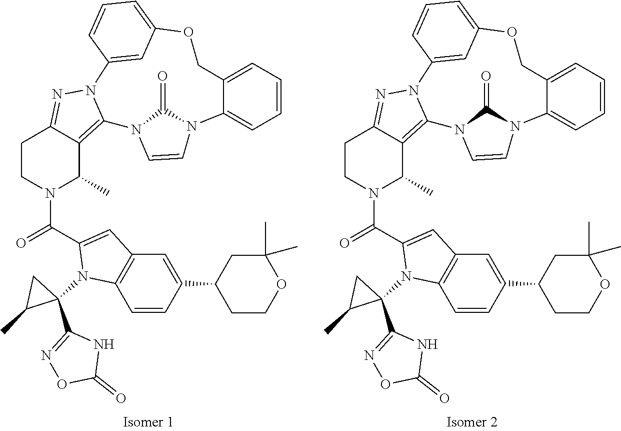

### Procedure for Synthesis of Compound 21

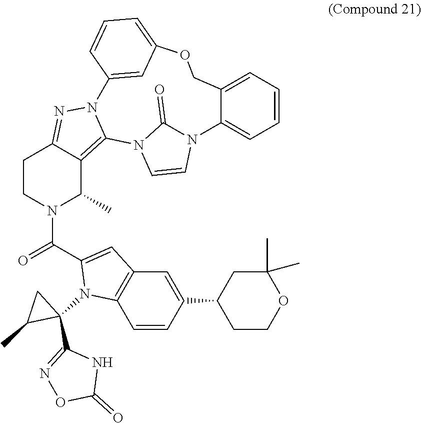

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaen-28-one

Step A: To the solution of 3-nitrophenol (3 g, 21.6 mmol) in DMF (120 mL) was added sodium hydride (1.73 g, 43.1 mmol) at 0° C. under N₂. The reaction was stirred at 0° C. for 30 min. Then 1-bromo-2-(bromomethyl)benzene (6.47 g, 25.9 mmol) was added to the mixture. The reaction was allowed to warm to ambient temperature where it stirred for 2 h. The reaction mixture was quenched with saturated aq. NH₄Cl and extracted with ethyl acetate. The organic layer was washed with water (2×) and the aqueous layer was further extracted with ethyl acetate (2×). The combined organic layers were washed with brine solution, dried over anhydrous Na₂SO₄, filtered and evaporated under reduced pressure to give the crude compound, which was purified by column chromatography (0-10% EtOAc in PE) to give 1-bromo-2-((3-nitrophenoxy)methyl)benzene (6.7 g, 21.1 mmol) as a yellow solid. 1H NMR (400 MHz, DMSO) δ 7.87-7.83 (m, 2H), 7.71 (d, J=7.9 Hz, 1H), 7.64-7.60 (m, 2H), 7.53-7.51 (m, 1H), 7.47-7.42 (m, 1H), 7.36-7.32 (m, 1H), 5.26 (s, 2H).

Step B: To a solution of 1-bromo-2-((3-nitrophenoxy)methyl)benzene (6.7 g, 21.7 mmol) in ethanol (100 mL) and water (20 mL) was added iron (6.0 g, 109 mmol) and ammonium chloride (11.6 g, 217 mmol). The reaction was stirred at 80° C. for 16 h. The reaction mixture was cooled to ambient temperature and diluted with ethyl acetate, washed with water (2×) and the aqueous layer was extracted with ethyl acetate (3×). The combined organic layers were washed with brine solution, dried over anhydrous Na₂SO₄, filtered and evaporated under reduced pressure to give the crude compound. The crude compound was purified by column chromatography (0-6% MeOH in DCM) to give 3-((2-bromobenzyl)oxy) aniline (5.2 g, 17.8 mmol) as a yellow oil. m/z (ESI-pos) M+1=278.1/280.0

Step C: To a solution of 3-((2-bromobenzyl)oxy) aniline (5.2 g, 18.77 mmol) in 6M aq. HCl at 0° C. was added a solution of sodium nitrite (1.42 g, 20.64 mmol) in water (10 mL) dropwise. The mixture was then stirred at 0° C. for 1 h. A solution of SnCl₂ (7.1 g, 37.54 mmol) in con. HCl (30 mL) was added dropwise to the reaction mixture and then the mixture was allowed to warm to ambient temperature and stirred for 2 h. The pH was adjusted to ~14 with 4N aq. NaOH. The mixture was diluted with ethyl acetate, washed with water (2×) and the aqueous layer was extracted with ethyl acetate (2×). The combined organic layers were washed with brine solution, and dried over anhydrous Na₂SO₄., filtered and evaporated under reduced pressure. The residue was purified by column chromatography (0-10% MeOH in DCM) to give (3-((2-bromobenzyl)oxy)phenyl)hydrazine (3.05 g) as a yellow oil. m/z (ESI-pos) M+1=293.0/295.0

Step D: To a solution of (3-((2-bromobenzyl)oxy)phenyl)hydrazine (3.05 g, 10.44 mmol) in Ethanol (50 mL) was added tert-butyl(2S)-3-cyano-2-methyl-4-oxo-1-piperidinecarboxylate (2.48 g, 10.44 mmol). The resulting mixture was stirred at 80° C. for 16 hr. The reaction mixture was concentrated under reduced pressure to give the crude compound, which was purified by column chromatography (0-6% MeOH in DCM) to give tert-butyl (S)-3-amino-2-(3-((2-bromobenzyl)oxy)phenyl)-4-methyl-2,4,6,7-tetrahydro-5H-pyrazolo[4,3-c]pyridine-5-carboxylate (3.34 g, 5.85 mmol) as a yellow oil. m/z (ESI-pos) M+1=513.1/515.1

Step E: To a solution of tert-butyl (S)-3-amino-2-(3-((2-bromobenzyl)oxy)phenyl)-4-methyl-2,4,6,7-tetrahydro-5H-pyrazolo[4,3-c]pyridine-5-carboxylate (2.0 g, 3.90 mmol) and NaHCO₃ (982 mg, 11.7 mmol) in tetrahydrofuran (30 mL) was added benzenyl chloroformate (761 mg, 7.81 mmol). The reaction was stirred at 25° C. for 2 hr. The reaction mixture was concentrated to give tert-butyl (S)-2-(3-((2-bromobenzyl)oxy)phenyl)-4-methyl-3-((phenoxycarbonyl)amino)-2,4,6,7-tetrahydro-5H-pyrazolo[4,3-c]pyridine-5-carboxylate (2.2 g) as a crude yellow oil, which was used directly for the next step without any further purification. m/z (ESI-pos) M+1=633.1/635.1

Step F: Crude tert-butyl (S)-2-(3-((2-bromobenzyl)oxy)phenyl)-4-methyl-3-((phenoxy carbonyl)amino)-2,4,6,7-tetrahydro-5H-pyrazolo[4,3-c]pyridine-5-carboxylate (2.2 g crude, 3.90 mmol) was dissolved in pyridine (50 mL), to which 2,2-dimethoxyethylamine (1.23 g, 11.7 mmol) was added. The mixture was stirred at 25° C. for 3 hr. The reaction mixture was concentrated under reduced pressure and the crude compound was purified by column chromatography (0-5% MeOH in DCM) to give tert-butyl (S)-3-{3-[2,2-bis(methylamino)ethyl]ureido}-2-(m-{[(o-bromophenyl)methoxy]methyl}phenyl)-4-methyl-1,2,5-triaza-2,4,6,7-tetrahydro-5H-indene-5-carboxylate (1.5 g, 1.86 mmol) as a yellow oil. m/z (ESI-pos) M+1=644.2/646.2

Step G: To a solution of tert-butyl (S)-3-{3-[2,2-bis(methylamino)ethyl]ureido}-2-(m-{[(o-bromophenyl)methoxy]methyl}phenyl)-4-methyl-1,2,5-triaza-2,4,6,7-tetrahydro-5H-indene-5-carboxylate (1.5 g, 2.33 mmol) in tetrahydrofuran (50 mL) was added methanesulfonic acid (291 mg, 3.03 mmol). The mixture was stirred at 60° C. for 2 hr. Aqueous K₃PO₄ was added to adjust the pH to ~9, and then Boc2O (507 mg, 2.33 mmol) was added. The resulting mixture was stirred at 25° C. for 1 hr. The reaction mixture was diluted with ethyl acetate, washed with water (2×) and the aqueous layer was extracted with ethyl acetate (2×). The combined organic layers were washed with 1N aq. HCl (20 mL), followed by brine and then dried over anhydrous Na₂SO₄. The mixture was filtered and the filtrate was evaporated under reduced pressure to give the crude compound. The crude compound was purified by column chromatography (0-80% EtOAc in PE) to give tert-butyl (S)-2-(3-((2-bromo benzyl)oxy)phenyl)-4-methyl-3-(2-oxo-2,3-dihydro-1H-imidazol-1-yl)-2,4,6,7-tetrahydro-5H-pyrazolo[4,3-c]pyridine-5-carboxylate (850 mg, 1.32 mmol) as a yellow solid. m/z (ESI-pos) M+1=580.1/582.1

Step H: To a solution of tert-butyl (S)-2-(3-((2-bromobenzyl)oxy)phenyl)-4-methyl-3-(2-oxo-2,3-dihydro-1H-imidazol-1-yl)-2,4,6,7-tetrahydro-5H-pyrazolo[4,3-c]pyridine-5-carboxylate (1360 mg, 2.34 mmol) in N-methyl-2-pyrrolidone (80 mL) was added dipotassium carbonate (972 mg, 7.04 mmol), (1R,2R)-1,2-bis(methylamino)cyclohexane (332 mg, 2.34 mmol), copper iodide (445 mg, 2.34 mmol) at 25° C. under N₂. The resulting mixture was stirred at 130° C. for 16 hr. The reaction mixture was cooled to ambient temperature and diluted with ethyl acetate, washed with water (2×) and the aqueous layer was extracted with ethyl acetate (2×). The combined organic layers were washed with aq. LiCl solution and dried over anhydrous Na₂SO₄. The mixture was filtered and the filtrate was evaporated under reduced pressure. The obtained residue was purified by column chromatography (0-80% EtOAc in PE) to give tert-butyl (S)-8-methyl-28-oxo-22-oxa-2,3,7,11,14-pentaazahexacyclo[21.3.1.1¹¹,¹⁴.0²,¹⁰.0⁴,⁹.0¹⁵,²⁰]octacosa 1(27),3,9,12,15,17,19,23,25-nonaene-7-carboxylate (120 mg) as a yellow solid. m/z (ESI-pos) M+1=500.1

Step I: tert-butyl (S)-8-methyl-28-oxo-22-oxa-2,3,7,11,14-pentaazahexacyclo[21.3.1.1¹¹,¹⁴.0²,¹⁰.0⁴,⁹.0¹⁵,²⁰]octacosa 1(27),3,9,12,15,17,19,23,25-nonaene-7-carboxylate (120 mg, 240 μmol) was dissolved in HCl/1,4-dioxane (4M, 5 mL) and stirred at 25° C. for 1 h. The reaction mixture was directly concentrated to give the crude(S)-8-methyl-22-oxa-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaen-28-one hydrogen chloride (112 mg) as a yellow solid, which was used to the next step directly. m/z (ESI-pos) M+1=400.2

Step J: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (115 mg, 280 μmol), DIEA (109 mg, 841 μmol) in DMF (5 mL) was added HATU (160 mg, 421 μmol). The mixture was stirred at 25° C. for 20 minutes and(S)-8-methyl-22-oxa-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaen-28-one hydrogen chloride (112 mg, 280 μmol) was added. The resulting mixture was stirred at 25° C. for 16 hr, then diluted with ethyl acetate, washed with water (2×). The aqueous layer was extracted with ethyl acetate (2×). The combined organic layers were washed with brine solution, then dried over anhydrous Na₂SO₄, filtered and the filtrate was evaporated under reduced pressure to give the crude compound which was purified by column chromatography (0-10% MeOH in DCM) to give the desired product (130 mg), which was further purified by prep-HPLC [Instrument: GILSON Prep LC with UV detector; Column: XBridge Prep C18 19*150 mm 5 μm; Gradient: eluted with 70-95% ACN in H₂O (0.1% NH4HCO3) in 10 mins; Flow rate: 15 ml/min] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaen-28-one (30 mg) as yellow solid, which was dissolved in MeOH (100 mL) and separated by chiral supercritical fluid chromatography (separation condition: Daicel CHIRALPAK IC_3, 4.6 mm*250 mm 5 um; Mobile Phase: MeOH-DCM-DEA at 1 mL/min; Temp: 30° C.; Wavelength: 254 nm) to afford Isomer 1 (13.8 mg) and Isomer 2 (11.4 mg) as white solids. (The absolute configuration was unknown, the configuration of two atropisomers were assigned arbitrarily).

Isomer 1: m/z (ESI-pos) M+1=793.3; 1H NMR (400 MHz, DMSO) δ 11.67 (s, 1H), 7.74-6.69 (m, 1H), 7.53-7.41 (m, 4H), 7.38-7.34 (m, 1H), 7.26-7.17 (m, 3H), 7.11-7.02 (m, 2H), 6.87 (s, 1H), 6.66-6.61 (m, 1H), 6.18 (s, 1H), 5.83-5.66 (m, 1H), 5.54 (d, J=13.2 Hz, 1H), 4.94 (d, J=13.6 Hz, 1H), 4.54-4.38 (m, 1H), 3.73 (d, J=7.6 Hz, 2H), 3.60-3.49 (m, 1H), 3.00-2.87 (m, 2H), 1.86-1.50 (m, 11H), 1.28 (s, 3H), 1.24-1.12 (m, 6H).

Isomer 2: m/z (ESI-pos) M+1=793.3; 1H NMR (400 MHz, DMSO) δ 11.59 (s, 1H), 7.70 (d, J=7.6 Hz, 1H), 7.50-7.38 (m, 4H), 7.35-7.26 (m, 2H), 7.24-7.17 (m, 2H), 7.13-7.08 (m, 1H), 6.96 (s, 1H), 6.87 (s, 1H), 6.63 (d, J=8.0 Hz, 1H), 6.21 (s, 1H), 5.78-5.52 (m, 2H), 4.95 (d, J=13.6 Hz, 1H), 4.51-4.34 (m, 1H), 3.76-3.70 (m, 2H), 3.63-3.51 (m, 1H), 2.99-2.83 (m, 2H), 1.76-1.49 (m, 11H), 1.27 (s, 3H), 1.24-1.12 (m, 6H).

### Example 22—Synthesis of Compound 22

### Synthetic Scheme

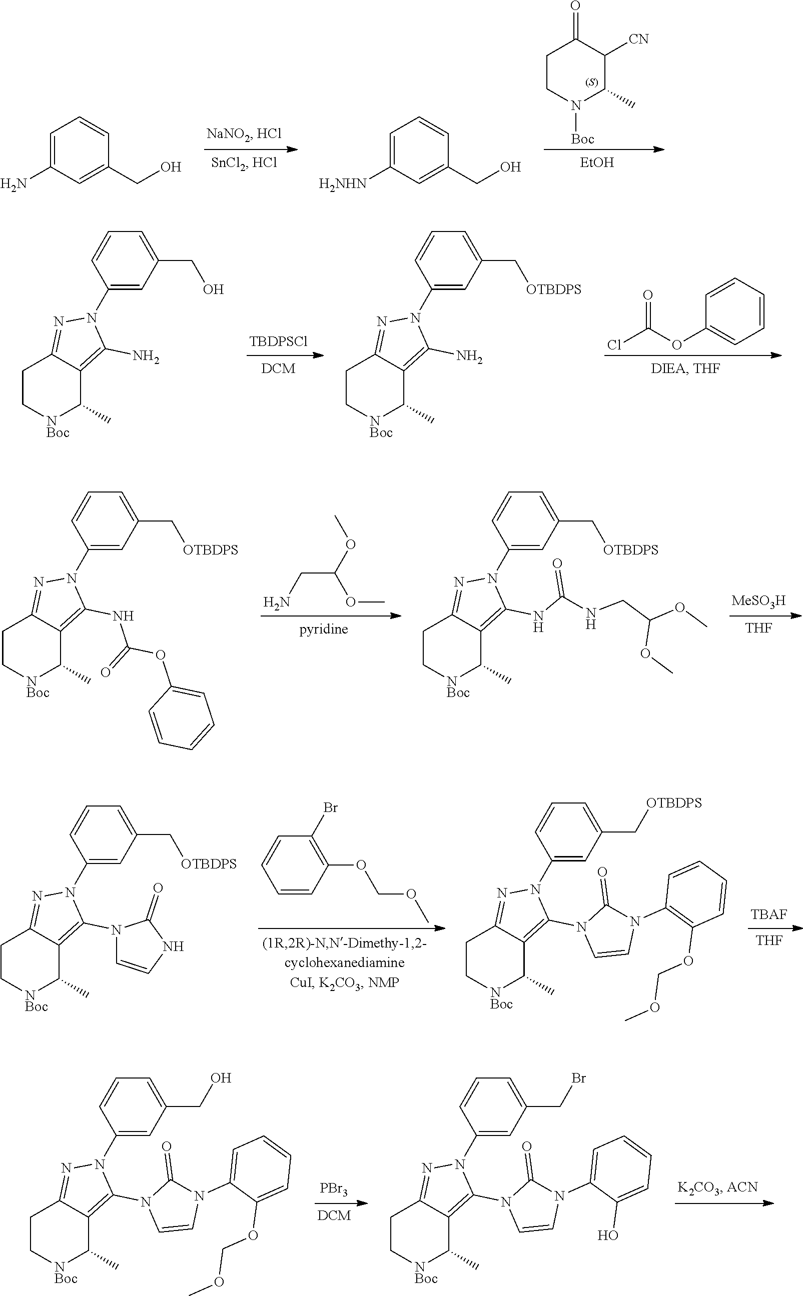

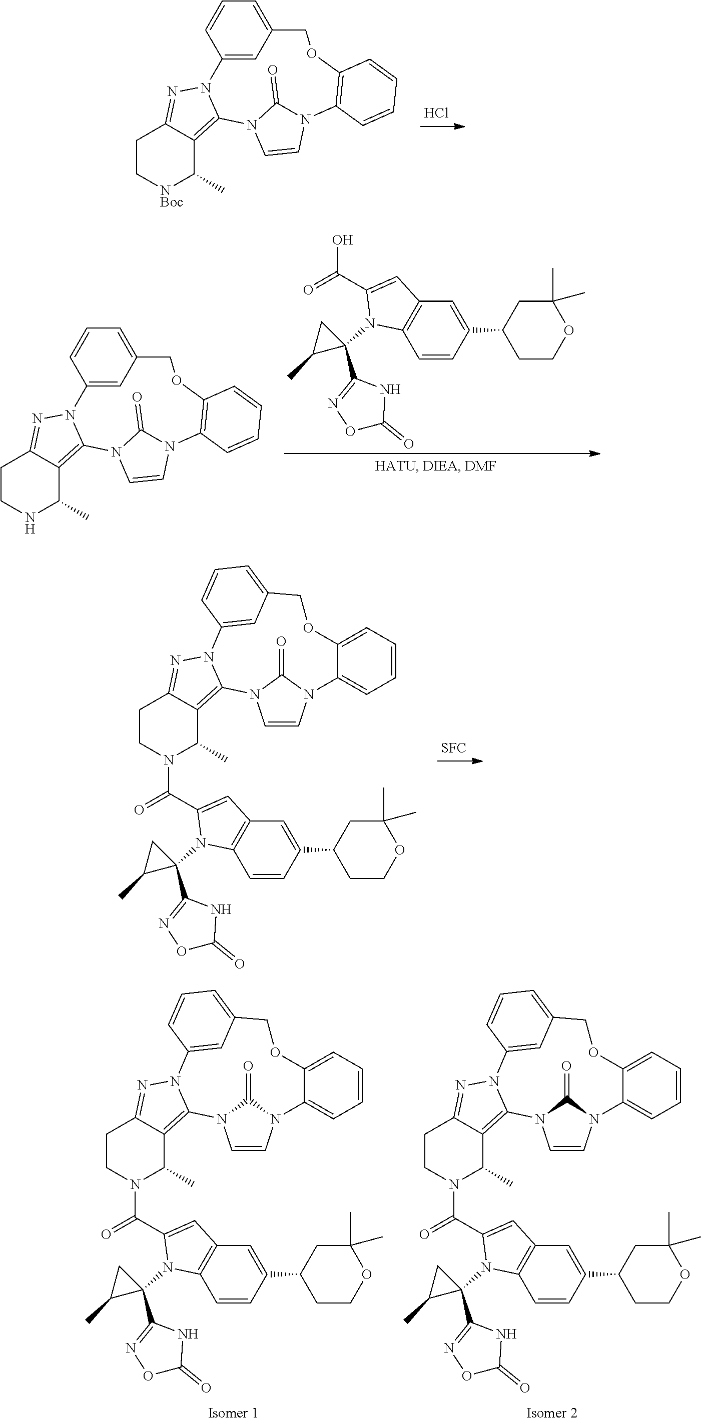

### Procedure for Synthesis of Compound 22

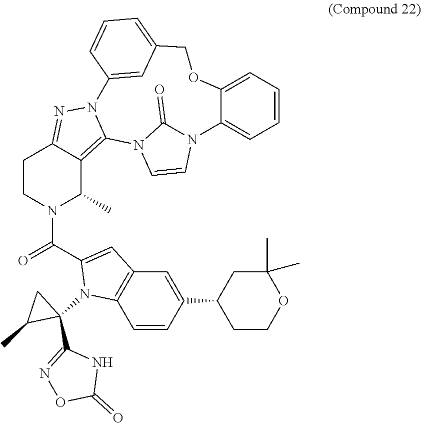

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-21-oxa-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaen-28-one

Step A: To a solution of (3-aminophenyl) methanol (4 g, 32.5 mmol) in conc. HCl (15 mL) and H₂O (15 mL) at 0° C. was added NaNO₂ (2.47 g, 35.7 mmol) in H₂O (5 mL), dropwise. After the mixture was stirred at 0° C. for 45 min, SnCl₂ (13.5 g, 71.5 mmol) in conc. HCl (15 mL) and H₂O (15 mL) was added dropwise and the mixture was stirred for 1 hour. The reaction was quenched with aqueous NaOH (10N, 35 mL) to adjust the pH to about 9. Then the mixture was extracted with EtOAc (3×) and the combined organic layers were dried over anhydrous Na₂SO₄, filtered and concentrated. The crude product was purified by column chromatography (0-100% EtOAc in PE) to give (3-hydrazinylphenyl) methanol (1.8 g, 11.7 mmol) as a yellow solid. m/z (ESI-pos) M+1=139.1

Step B: To a solution of (3-hydrazinylphenyl) methanol (1.8 g, 11.7 mmol) in EtOH (40 mL) was added tert-butyl(2S)-3-cyano-2-methyl-4-oxopiperidine-1-carboxylate (2.79 g, 11.7 mmol). The mixture was stirred at 80° C. for 8 hours. The reaction mixture was concentrated under reduced pressure and the crude residue was purified by column chromatography (0-100 EtOAc in PE) to give tert-butyl (S)-3-amino-2-(3-(hydroxymethyl)phenyl)-4-methyl-2,4,6,7-tetrahydro-5H-pyrazolo[4,3-c]pyridine-5-carboxylate (3.4 g, 8.54 mmol) as a yellow solid. m/z (ESI-pos) M+1=359.2.

Step C: To a solution of tert-butyl (S)-3-amino-2-(3-(hydroxymethyl)phenyl)-4-methyl-2,4,6,7-tetrahydro-5H-pyrazolo[4,3-c]pyridine-5-carboxylate (3.4 g, 8.54 mmol) in DCM (50 mL) at 0° C. was added imidazole (1.16 g, 17.1 mmol) and tert-butyl(chloro)bis(phenyl) silane (2.58 g, 9.39 mmol). The mixture was stirred at 20° C. for 2 hours. The reaction mixture was quenched with H₂O and extracted with DCM (2×). The combined organics were dried over Na₂SO₄, filtered and concentrated. The crude product was purified by column chromatography (0-25% EtOAc in PE) to give tert-butyl (S)-3-amino-2-(m-{[tert-butylbis(phenyl)siloxy]methyl}phenyl)-4-methyl-1,2,5-triaza-2,4,6,7-tetrahydro-5H-indene-5-carboxylate (5 g, 7.54 mmol) as a yellow solid. m/z (ESI-pos) M+1=597.3.

Step D: To a solution of tert-butyl (S)-3-amino-2-(m-{[tert-butylbis(phenyl)siloxy]methyl}phenyl)-4-methyl-1,2,5-triaza-2,4,6,7-tetrahydro-5H-indene-5-carboxylate (5 g, 7.54 mmol) in tetrahydrofuran (50 mL) at 0° C. was added DIEA (2.92 g, 22.6 mmol) and benzenyl chloroformate (2.36 g, 15.1 mmol). The mixture was stirred at ambient temperature for 2 hours. Then the mixture was concentrated to get crude tert-butyl (S)-2-(3-(((tert-butyldiphenylsilyl)oxy)methyl)phenyl)-4-methyl-3-((phenoxycarbonyl)amino)-2,4,6,7-tetrahydro-5H-pyrazolo[4,3-c]pyridine-5-carboxylate (7.7 g, 7.52 mmol) as a yellow solid, that was used directly in the subsequent step. m/z (ESI-pos) M+1=717.3.

Step E: To a solution of tert-butyl (S)-2-(3-(((tert-butyldiphenylsilyl)oxy)methyl)phenyl)-4-methyl-3-((phenoxycarbonyl)amino)-2,4,6,7-tetrahydro-5H-pyrazolo[4,3-c]pyridine-5-carboxylate (7.7 g, 7.52 mmol) in pyridine (50 mL) was added 2,2-dimethoxyethylamine (2.37 g, 22.6 mmol) and the mixture was stirred at ambient temperature for 2 hours. The reaction mixture was concentrated under reduced pressure and the crude product was purified by column chromatography (0-50% EtOAc in PE) to give tert-butyl (S)-2-(3-(((tert-butyldiphenylsilyl)oxy)methyl)phenyl)-3-(3-(2,2-dimethoxyethyl)ureido)-4-methyl-2,4,6,7-tetrahydro-5H-pyrazolo[4,3-c]pyridine-5-carboxylate (5.5 g, 6.8 mmol) as a white solid. m/z (ESI-pos) M+1=728.3.

Step F: To a solution of tert-butyl (S)-2-(3-(((tert-butyldiphenylsilyl)oxy)methyl)phenyl)-3-(3-(2,2-dimethoxyethyl)ureido)-4-methyl-2,4,6,7-tetrahydro-5H-pyrazolo[4,3-c]pyridine-5-carboxylate (5.5 g, 6.8 mmol) in tetrahydrofuran (200 mL) was added methanesulfonic acid (784 mg, 8.16 mmol) and the mixture was stirred at 60° C. for 1 hour. The reaction mixture was cooled to ambient temperature and diluted with H₂O and then extracted with EtOAc (2×). The combined organic layers were dried over anhydrous Na₂SO₄, filtered and the filtrate was concentrated under reduced pressure. The crude product was purified by column chromatography (0-100% EtOAc in PE) to give tert-butyl(S)-2-(3-(((tert-butyldiphenylsilyl)oxy)methyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (4.1 g, 5.56 mmol) as a white solid. m/z (ESI-pos) M+1=664.3.

Step G: To a solution of tert-butyl (S)-2-(3-(((tert-butyldiphenylsilyl)oxy)methyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (2 g, 3.01 mmol) in N-methyl-2-pyrrolidone (40 mL) was added o-bromomethoxymethoxybenzene (981 mg, 4.52 mmol), copper iodide (287 mg, 1.51 mmol), (1R,2R)-1,2-bis(methylamino)cyclohexane (429 mg, 3.01 mmol) and tripotassium phosphate (1.92 g, 9.04 mmol). The mixture was stirred at 100° C. for 16 hours. The reaction mixture was cooled to ambient temperature and diluted with H₂O then extracted with EtOAc (2×). The combined EtOAc layers were washed with aqueous LiCl (2×), dried over Na₂SO₄, filtered and concentrated in vacuo. The crude product was purified by column chromatography (0-50% EtOAc in PE) to give tert-butyl (S)-2-(3-(((tert-butyldiphenylsilyl)oxy)methyl)phenyl)-3-(3-(2-(methoxy methoxy)phenyl)-2-oxo-2,3-dihydro-1H-imidazol-1-yl)-4-methyl-2,4,6,7-tetrahydro-5H-pyrazolo[4,3-c]pyridine-5-carboxylate (2.2 g, 2.47 mmol) as a white solid. m/z (ESI-pos) M+1=800.3.

Step H: To a solution of tert-butyl (S)-2-(3-(((tert-butyldiphenylsilyl)oxy)methyl)phenyl)-3-(3-(2-(methoxymethoxy)phenyl)-2-oxo-2,3-dihydro-1H-imidazol-1-yl)-4-methyl-2,4,6,7-tetrahydro-5H-pyrazolo[4,3-c]pyridine-5-carboxylate (2.2 g, 2.47 mmol) in tetrahydrofuran (30 mL) at 0° C. was added TBAF (1.0M in THF, 3.71 mL, 3.71 mmol). The mixture was allowed to warm to ambient temperature and stirred for 1 hour. The reaction mixture was diluted H₂O and extracted with EtOAc (2×). The combined EtOAc layers were washed with brine, dried over Na₂SO₄, filtered and concentrated in vacuo. The crude product was purified by column chromatography (0-60% EtOAc in PE) to give tert-butyl 2-(3-(3-hydroxypropyl)phenyl)-3-(3-(2-(methoxymethoxy)phenyl)-2-oxo-2,3-dihydro-1H-imidazol-1-yl)-2,6,7,8-tetrahydro pyrazolo[4,3-c]azepine-5(4H)-carboxylate (1.3 g, 2.08 mmol) as a white solid. m/z (ESI-pos) M+1=562.2

Step I: To a solution of tert-butyl 2-(3-(3-hydroxypropyl)phenyl)-3-(3-(2-(methoxymethoxy)phenyl)-2-oxo-2,3-dihydro-1H-imidazol-1-yl)-2,6,7,8-tetrahydro pyrazolo[4,3-c]azepine-5(4H)-carboxylate (1.3 g, 2.08 mmol) in DCM (50 mL) at 0° C. was added tribromophosphane (338 mg, 1.25 mmol). The mixture was stirred at 0° C. for 1 hour. Aqueous NaHCO₃ solution was added to adjust pH to about 8 and Boc₂O (544 mg, 2.5 mmol) was added, the mixture was stirred at 20° C. for 1 hour. The mixture was diluted with H₂O and extracted with EtOAc (2×). The combined EtOAc layers were washed with brine, dried over Na₂SO₄, filtered and the filtrate was concentrated in vacuo. The crude residue was purified by column chromatography (0-50% EtOAc in PE) to give tert-butyl (S)-2-(3-(bromomethyl)phenyl)-3-(3-(2-hydroxyphenyl)-2-oxo-2,3-dihydro-1H-imidazol-1-yl)-4-methyl-2,4,6,7-tetrahydro-5H-pyrazolo[4,3-c]pyridine-5-carboxylate (440 mg, 682 μmol) as a white solid. m/z (ESI-pos) M+1=580.1.

Step J: To a solution of tert-butyl (S)-2-[m-(bromomethyl)phenyl]-3-[3-(o-hydroxyphenyl)-2-oxo-2,3-dihydro-1H-imidazol-1-yl]-4-methyl-1,2,5-triaza-2,4,6,7-tetrahydro-5H-indene-5-carboxylate (440 mg, 682 μmol) in ACN (50 mL) was added dipotassium carbonate (283 mg, 2.05 mmol) and the mixture was stirred at ambient temperature for 16 hours. The reaction mixture was diluted with H₂O and extracted with EtOAc (2×). The combined EtOAc layers were washed with brine, dried over Na₂SO₄, filtered and concentrated in vacuo. The crude product was purified by column chromatography (0-50% EtOAc in PE) to give tert-butyl (S)-8-methyl-28-oxo-21-oxa-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaene-7-carboxylate (270 mg, 486 μmol) as a white solid. m/z (ESI-pos) M+1=500.2.

Step K: A solution of tert-butyl (S)-8-methyl-28-oxo-21-oxa-2,3,7,11,14-pentaazahexacyclo[21.3.1.1¹¹,¹⁴.0²,¹⁰.0⁴,⁹.0¹⁵,²⁰]octacosa1(27),3,9,12,15(20), 16,18,23,25-nonaene-7-carboxylate (270 mg, 486 μmol) in HCl/1,4-dioxane (4M, 5 mL) was stirred at ambient temperature for 2 hours. The mixture was concentrated directly in vacuo to get crude(S)-8-methyl-21-oxa-2,3,7,11,14-pentaazahexacyclo[21.3.1.1¹¹,¹⁴.0²,¹⁰.0⁴,⁹.0¹⁵,²⁰]octacosa-1(27),3,9,12,15(20),16,18,23,25-nonaen-28-one hydrogen chloride (215 mg, 484 μmol) as a white solid. m/z (ESI-pos) M+1=400.2.

Step L: To a solution of(S)-8-methyl-21-oxa-2,3,7,11,14-pentaazahexacyclo[21.3.1.1¹¹,¹⁴.0²,¹⁰.0⁴,⁹.0¹⁵,²⁰]octacosa1(27),3,9,12,15(20),16,18,23,25-nonaen-28-one hydrogen chloride (215 mg, 484 μmol) in DMF (10.8 mL) was added 5-[(S)-2,2-dimethyltetrahydro-4H-pyran-4-yl]-1-[(2S)-2-methyl-1-(5-oxo-4,5-dihydro-1,2,4-oxadiazol-3-yl)cyclo propyl]-2-indolecarboxylic acid (199 mg, 484 μmol) and DIEA (250 mg, 1.94 mmol) and the reaction mixture solution was stirred at 20° C. for 10 mins. Then HATU (239 mg, 630 μmol) was added and the mixture was stirred for 16 hours. H₂O was then added and the mixture was extracted with EtOAc (2×). The combined EtOAc layers were washed with brine, dried over Na₂SO₄, filtered and concentrated in vacuo. The crude product was purified by column chromatography (0-5% MeOH in DCM) to give 300 mg of product, which was further purified by prep-HPLC [Instrument: GILSON Prep LC with UV detector; Column: XBridge Prep C18 19*150 mm 5 μm; Gradient: eluted with 75-95% ACN in H₂O (0.1% FA)] to give (S)-7-({5-[(S)-2,2-dimethyltetrahydro-4H-pyran-4-yl]-1-[(2S)-2-methyl-1-(5-oxo-4,5-dihydro-1,2,4-oxadiazol-3-yl)cyclopropyl]-2-indolyl}carbonyl)-8-methyl-21-oxa-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaen-28-one (140 mg, 168 μmol) as a white solid. m/z (ESI-pos) M+1=793.3.

Step M: A solution of(S)-7-({5-[(S)-2,2-dimethyltetrahydro-4H-pyran-4-yl]-1-[(2S)-2-methyl-1-(5-oxo-4,5-dihydro-1,2,4-oxadiazol-3-yl)cyclopropyl]-2-indolyl}carbonyl)-8-methyl-21-oxa-2,3,7,11,14-pentaazahexacyclo[21.3.1.1¹¹,¹⁴.0²,¹⁰.0⁴,⁹.0¹⁵,²⁰]octacosa-1(27),3,9,12,15,17,19,23,25-nonaen-28-one (140 mg, 177 μmol) in MeOH (20 mL) was separated by chiral supercritical fluid chromatography (separation condition: Daicel CHIRALCEL IC, 250 mm*30 mm I.D., 10 μm; Mobile Phase: CO2/MeOH [0.2% NH₃ (7M Solution in MeOH)]=90/10 at 25 mL/min; Temp: 35° C.; Wavelength: 230 nm) to afford Isomer 1 (60 mg) and Isomer 2 (50 mg), which were further purified by prep-HPLC [Instrument: GILSON Prep LC with UV detector; Column: XBridge Prep C18 19*150 mm 5 μm; Gradient: eluted with 70-95% ACN in H₂O (0.1% FA) in 10 mins; Flow rate: 15 mL/min] to give pure Isomer 1 (46.3 mg) and Isomer 2 (33.0 mg) as white solids (The absolute configuration was unknown, the configuration of two atropisomers were assigned arbitrarily).

Isomer 1: m/z (ESI-pos) M+1=793.3; ¹HNMR (400 MHz, DMSO) δ 11.65 (s, 1H), 7.53 (s, 1H), 7.48-7.40 (m, 4H), 7.32-7.24 (m, 3H), 7.11-7.03 (m, 2H), 6.96-6.80 (m, 4H), 5.72 (s, 1H), 5.40 (d, J=13.6 Hz, 1H), 5.07 (d, J=14.0 Hz, 1H), 4.46 (s, 1H), 3.73-3.71 (m, 2H), 3.58 (s, 1H), 3.01-2.99 (m, 2H), 2.90 (d, J=13.6 Hz, 1H), 1.82-1.65 (m, 5H), 1.62-1.50 (m, 5H), 1.28 (s, 3H), 1.19 (s, 6H)

Isomer 2: m/z (ESI-pos) M+1=793.3; ¹HNMR (400 MHz, DMSO) δ 11.60 (s, 1H), 7.51-7.38 (m, 5H), 7.32-7.23 (m, 3H), 7.10-6.99 (m3H), 6.87-6.82 (m, 3H), 5.62 (s, 1H), 5.41 (d, J=14.0 Hz, 1H), 5.06 (d, J=14.0 Hz, 1H), 4.44 (s, 1H), 3.73-3.71 (m, 2H), 3.60 (s, 1H), 3.03-2.99 (m, 2H), 2.92-2.88 (m, 1H), 1.73-1.65 (m, 7H), 1.62-1.50 (m, 3H), 1.28 (s, 3H), 1.19 (s, 6H).

### Example 23—Synthesis of Compound 23

### Synthetic Scheme

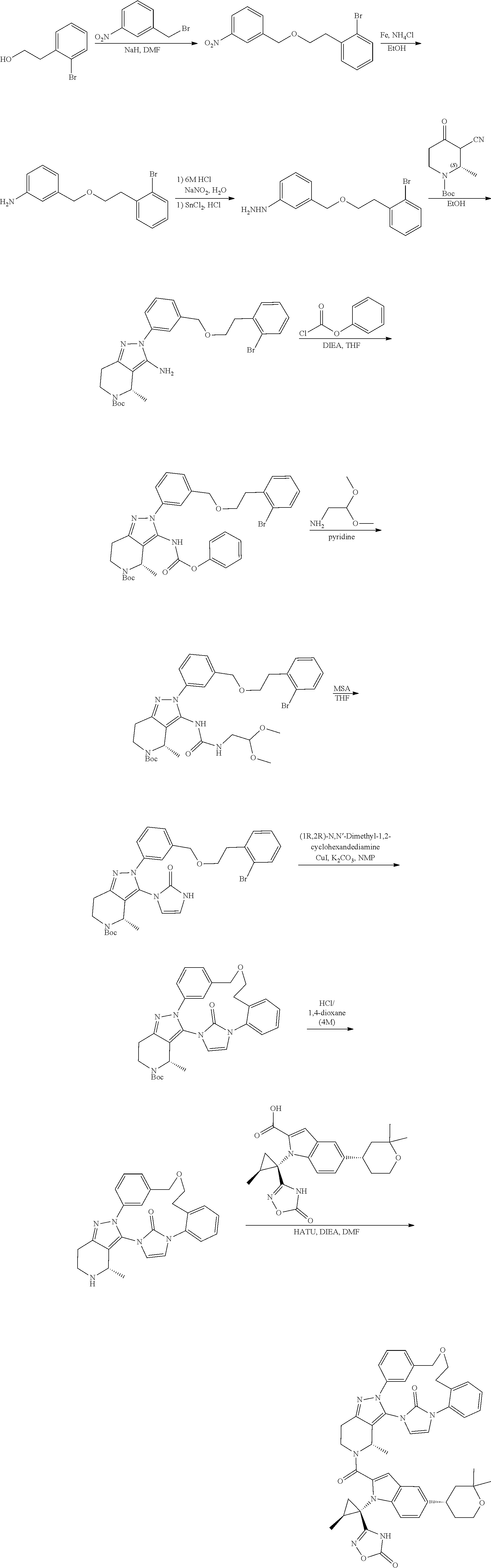

### Procedure for Synthesis of Compound 23

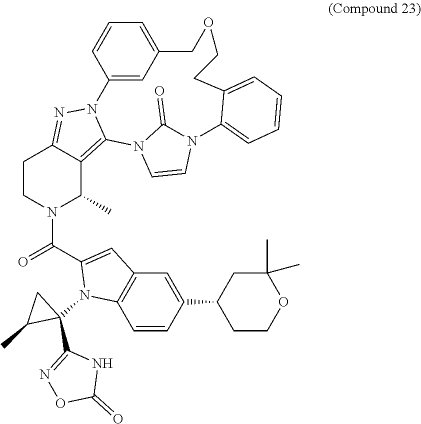

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴,0²,¹⁰,0⁴,⁹.0¹⁵,²⁰]triaconta-1(29),3,9,12,15,17,19,25,27-30-one

Step A: To a solution of 2-(o-bromophenyl) ethanol (3 g, 14.9 mmol) in DMF (30 mL) was added sodium hydride 60% w/w (1.19 g, 29.8 mmol) at 0° C. under N₂. The mixture was stirred at 0° C. for 30 min. Then m-(bromomethyl) nitrobenzene (3.87 g, 17.9 mmol) was added to the mixture. The reaction was allowed to warm to room temperature and was stirred for 2 hours. The reaction mixture was then quenched with saturated aqueous NH₄Cl, diluted with ethyl acetate, washed with water (2×) and the aqueous layer was extracted with ethyl acetate (2×). The combined ethyl acetate layers were washed with brine, dried over Na₂SO₄, filtered and then concentrated in vacuo. The crude product was purified by column chromatography (0-15% ethyl acetate in pet. ether). to give 1-bromo-2-(2-((3-nitrobenzyl)oxy)ethyl)benzene (4.8 g, 12.9 mmol) as a yellow oil. 1HNMR (400 MHz, CDCl₃) δ 8.16 (s, 1H), 8.12 (d, J=8.4 Hz, 1H), 7.60 (d, J=7.6 Hz, 1H), 7.55-7.46 (m, 2H), 7.31-7.23 (m, 2H), 7.11-7.07 (m, 1H), 4.61 (s, 2H), 3.76 (t, J=6.8 Hz, 2H), 3.11 (t, J=6.8 Hz, 2H).

Step B: To the solution of o-bromo {2-[(m-nitrophenyl)methoxy]ethyl}benzene (4.8 g, 12.9 mmol) in EtOH (80 mL) was added H₂O (20 mL), iron (3.59 g, 64.3 mmol) and ammonium chloride (3.44 g, 64.3 mmol) under N₂. The mixture was stirred at 70° C. for 2 hours. The reaction mixture was cooled to room temperature and quenched with H₂O, and then extracted with ethyl acetate (2×). The combined ethyl acetate layers were washed with brine, then dried over Na₂SO₄, filtered and concentrated. The crude product was purified by column chromatography (0-33% ethyl acetate in pet. ether) to give 3-((2-bromophenethoxy)methyl)aniline (4.1 g, 12.1 mmol) as a yellow oil. m/z (ESI-pos) M+1=306.0/308.0.

Step C: To a solution of m-{[2-(o-bromophenyl) ethoxy]methyl}aniline (2 g, 5.88 mmol) in conc. HCl (15 mL) and H₂O (15 mL) at 0° C. was added NaNO₂ (446 mg, 6.47 mmol) in H₂O (5 mL) dropwise. The mixture was stirred at 0° C. for 45 minutes, and then SnCl₂ (2.45 g, 12.9 mmol) in conc. HCl (15 mL) and H₂O (15 mL) was added dropwise, and the mixture was stirred at room temperature for another 1 hour. The reaction was quenched with aq. NaOH (10N, 35 mL) to adjust the pH to ~9. Then the mixture was extracted with ethyl acetate (3×), and the combined organic layers were dried over anhydrous Na₂SO₄, filtered, and concentrated under vacuum. The crude product was purified by column chromatography (0-100% ethyl acetate in pet. ether) to give (3-((2-bromophenethoxy)methyl)phenyl)hydrazine (1 g, 2.8 mmol) as a yellow solid. m/z (ESI-pos) M+1=321.1/323.1

Step D: To a solution of (3-((2-bromophenethoxy)methyl)phenyl)hydrazine (1 g, 2.8 mmol) in EtOH (20 mL) was added tert-butyl(2S)-3-cyano-2-methyl-4-oxo-1-piperidinecarboxylate (668 mg, 2.8 mmol) and the mixture was stirred at 80° C. for 16 hours. The mixture was concentrated under reduced pressure to get the crude product, which was purified by column chromatography (0-33% ethyl acetate in pet. ether) to give tert-butyl (S)-3-amino-2-(3-((2-bromophenethoxy)methyl)phenyl)-4-methyl-2,4,6,7-tetrahydro-5H-pyrazolo[4,3-c]pyridine-5-carboxylate (1 g, 1.66 mmol) as a yellow solid m/z (ESI-pos) M+1=541.1/543.2

Step E: To a solution of tert-butyl (S)-3-amino-2-(m-{[2-(o-bromophenyl) ethoxy]methyl}phenyl)-4-methyl-1,2,5-triaza-2,4,6,7-tetrahydro-5H-indene-5-carboxylate (1 g, 1.66 mmol) in tetrahydrofuran (9.58 mL) at 0° C. was added DIEA (644 mg, 4.99 mmol) and benzenyl chloroformate (520 mg, 3.32 mmol). The mixture was stirred at room temperature for 2 hours and then concentrated to get crude tert-butyl (S)-2-(3-((2-bromophenethoxy)methyl)phenyl)-4-methyl-3-((phenoxycarbonyl)amino)-2,4,6,7-tetrahydro-5H-pyrazolo[4,3-c]pyridine-5-carboxylate (1.8 g, 1.63 mmol) as a yellow solid, which was used directly in next step. m/z (ESI-pos) M+1=661.2/663.2

Step F: To a solution of crude tert-butyl (S)-2-(m-{[2-(o-bromophenyl) ethoxy]methyl}phenyl)-4-methyl-3-[phenyl(oxycarbonylamino)]-1,2,5-triaza-2,4,6,7-tetrahydro-5H-indene-5-carboxylate (1.8 g, 1.63 mmol) in pyridine (10 mL) was added 2,2-dimethoxyethylamine (515 mg, 4.9 mmol). The mixture was stirred at room temperature for 2 hours and then concentrated under vacuum. The obtained residue was purified by column chromatography (0-100% ethyl acetate in pet. ether) to give tert-butyl (S)-2-(3-((2-bromophenethoxy)methyl)phenyl)-3-(3-(2,2-dimethoxyethyl)ureido)-4-methyl-2,4,6,7-tetrahydro-5H-pyrazolo[4,3-c]pyridine-5-carboxylate (1.1 g, 1.47 mmol) as a white solid. m/z (ESI-pos) M+1=672.2/674.2

Step G: To a of tert-butyl (S)-2-(m-{[2-(0-solution bromophenyl)ethoxy]methyl}phenyl)-3-[3-(2,2-dimethoxyethyl)ureido]-4-methyl-1,2,5-triaza-2,4,6,7-tetrahydro-5H-indene 5-carboxylate (1.1 g, 1.47 mmol) in tetrahydrofuran (10 mL) was added methanesulfonic acid (170 mg, 1.77 mmol). The mixture was stirred at 60° C. for 1 hour and then cooled to room temperature. The reaction mixture was diluted with H₂O and extracted with ethyl acetate (2×). The combined organic layers were dried over anhydrous Na2SO4 and concentrated under vacuum to get the crude product, which was purified by column chromatography (0-100% ethyl acetate in pet. ether) to give tert-butyl (S)-2-(3-((2-bromophenethoxy)methyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (890 mg, 1.32 mmol) as a white solid. m/z (ESI-pos) M+1=608.2/610.2

Step H: To a solution of tert-butyl (S)-2-(3-((2-bromophenethoxy)methyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (890 mg, 1.32 mmol) in NMP (80 mL) was added copper iodide (125 mg, 658 μmol), (1R,2R)-1,2-bis(methylamino)cyclohexane (187 mg, 1.32 mmol) and dipotassium carbonate (546 mg, 3.95 mmol). The mixture was stirred at 100° C. for 16 hours and then cooled to room temperature, diluted with H₂O and extracted with ethyl acetate (2×). The combined ethyl acetate layers were washed with aqueous LiCl (2×), then dried over Na₂SO₄, filtered and concentrated in vacuo. The crude product was purified by column chromatography (0-50% ethyl acetate in pet. ether) to give tert-butyl (S)-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (320 mg, 546 μmol) as a white solid. m/z (ESI-pos) M+1=528.3

Step I: A solution of tert-butyl (S)-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (130 mg, 222 μmol) in HCl/1,4-dioxane (4M, 5 mL) was stirred at room temperature for 1 hour. The mixture was concentrated under vacuum to give crude(S)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (103 mg, 217 μmol) as a white solid. m/z (ESI-pos) M+1=428.2

Step J: To a solution of(S)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (103 mg, 217 μmol) in DMF (5 mL) was added 5-[(S)-2,2-dimethyltetrahydro-4H-pyran-4-yl]-1-[(2S)-2-methyl-1-(5-oxo-4,5 dihydro-1,2,4-oxadiazol-3-yl)cyclopropyl]-2-indolecarboxylic acid (89.2 mg, 217 μmol) and DIEA (112 mg, 867 μmol) and the mixture was stirred at room temperature for 5 minutes, then HATU (124 mg, 325 μmol) was added and the mixture was stirred at room temperature for 16 hours. Then H₂O was added and extracted with ethyl acetate (2×). The combined ethyl acetate layers were washed with brine, then dried over Na₂SO₄ and concentrated in vacuo to get the crude product, which was purified by column chromatography (0-5% MeOH in DCM) to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (150 mg, 164 μmol) as a yellow solid. m/z (ESI-pos) M+1=821.3. ¹H NMR (400 MHz, DMSO) δ 11.61 (s, 1H), 7.61-7.45 (m, 3H), 7.44-7.19 (m, 7H), 7.14-6.62 (m, 4H), 5.64 (s, 1H), 4.70-4.32 (m, 3H), 3.76-3.68 (m, 2H), 3.67-3.51 (m, 1H), 3.42-3.22 (m, 2H), 3.20-3.09 (m, 1H), 3.03-2.75 (m, 4H), 1.93-1.43 (m, 10H), 1.32-1.09 (m, 9H).

### Example 24—Synthesis of Compound 24

### Synthetic Scheme

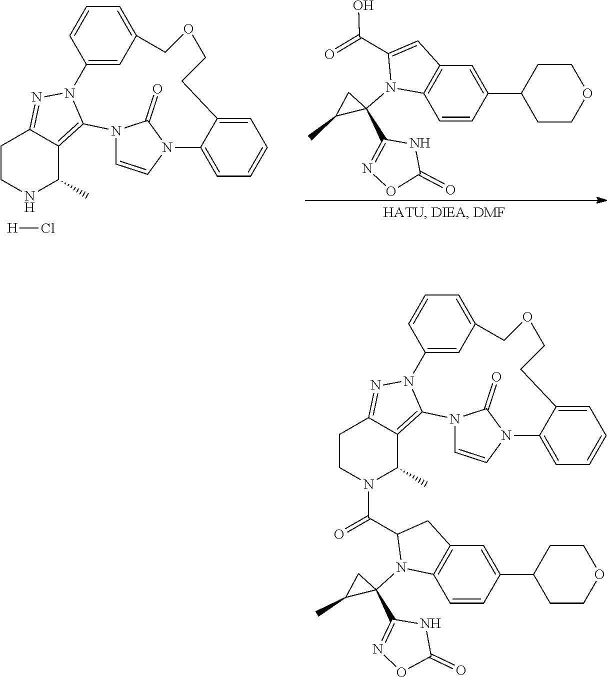

### Procedure for Synthesis of Compound 24

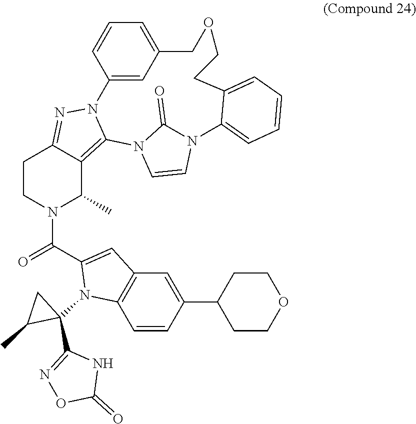

### (S)-8-methyl-7-({1-[(1S,2S)-2-methyl-1-(5-oxo-4,5-dihydro-1,2,4-oxadiazol-3-yl)cyclopropyl]-5-(tetrahydro-4H-pyran-4-yl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a mixture of 1-[(1S,2S)-2-methyl-1-(5-oxo-4,5-dihydro-1,2,4-oxadiazol-3-yl)cyclopropyl]-5-(tetrahydro-4H-pyran-4-yl)-2-indolecarboxylic acid (67.3 mg, 175 μmol), (S)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (75 mg, 175 μmol) and DIEA (227 mg, 1.75 mmol) in DMF (10 mL) was added HATU (0.1 g, 263 μmol) under N₂. The resulting mixture was stirred at room temperature for 16 hours, and then the reaction was quenched with water, and extracted with EtOAc (2×). The combined organic layers were washed with brine, dried over Na₂SO₄, filtered and concentrated. The crude residue was purified by Prep-HPLC [Waters MS-triggered Prep-LC with SQD2 detector, Welch Ultimate AQ-C18 10 um 21.2*250 mm, eluted with 70-100% ACN in H₂O (0.1% FA), flow rate: 25 mL/min] to give (S)-8-methyl-7-({1-[(1S,2S)-2-methyl-1-(5-oxo-4,5-dihydro-1,2,4-oxadiazol-3-yl)cyclopropyl]-5-(tetrahydro-4H-pyran-4-yl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (45.3 mg) as a white solid. m/z (ESI-pos) M+1=793.2; ¹H NMR (400 MHz, DMSO) δ 11.66 (s, 1H), 7.61-7.45 (m, 3H), 7.44-7.18 (m, 7H), 7.14-7.01 (m, 1H), 6.99-6.60 (m, 3H), 5.64 (s, 1H), 4.72-4.30 (m, 3H), 4.01-3.91 (m, 2H), 3.69-3.40 (m, 3H), 3.39-3.22 (m, 2H), 3.19-3.08 (m, 1H), 3.02-2.76 (m, 4H), 1.92-1.39 (m, 10H), 1.31-1.07 (m, 3H).

### Example 25—Synthesis of Compound 25

### Synthetic Scheme

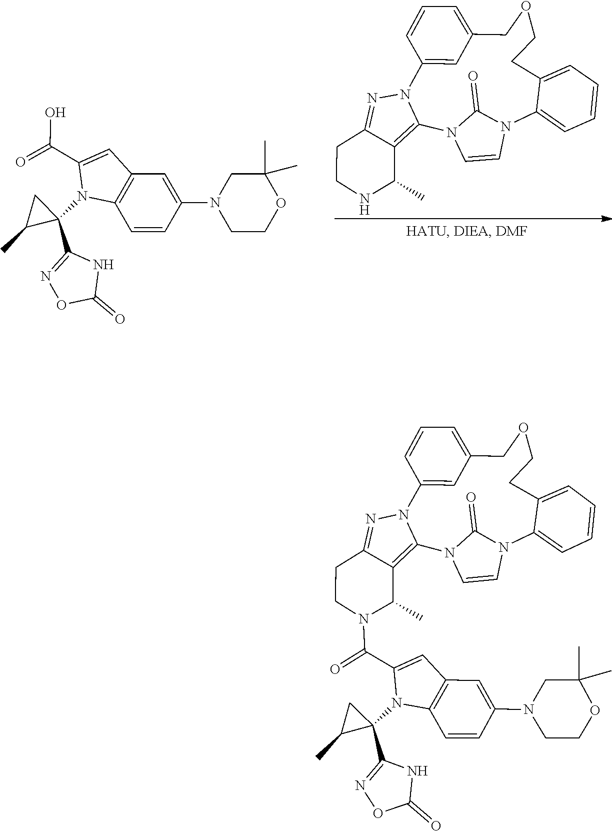

### Procedure for Synthesis of Compound 25

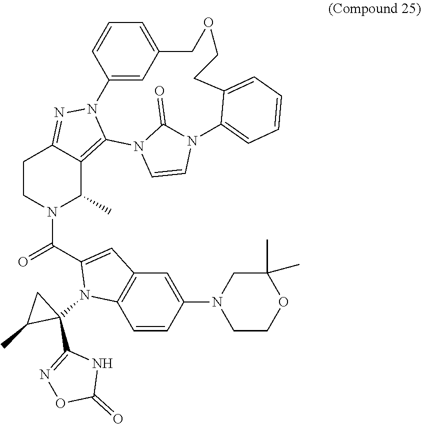

### (S)-7-{[5-(2,2-dimethyl-4-morpholinyl)-1-[(1S,2S)-2-methyl-1-(5-oxo-4,5-dihydro-1,2,4-oxadiazol-3-yl)cyclopropyl]-2-indolyl]carbonyl}-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a mixture of 5-(2,2-dimethylmorpholino)-1-((1S,2S)-2-methyl-1-(5-oxo-4,5-dihydro-1,2,4-oxadiazol-3-yl)cyclopropyl)-1H-indole-2-carboxylic acid (122 mg, 295 μmol), (S)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (126 mg, 295 μmol) and N-ethyldiisopropylamine (381 mg, 2.95 mmol) in DMF (10 mL) was added HATU (168 mg, 442 μmol) under N₂ and then stirred at room temperature for 16 h. The reaction was quenched with water, extracted with EtOAc (2×). The combined organic layers were washed with brine, dried over Na₂SO₄, filtered and the filtrate was concentrated to give a residue, which was purified by Prep-HPLC [Waters MS-triggered Prep-LC with QDA detector, WELCH ultimate C18 21.2*250 mm 10 um, eluted with 65-100% ACN in H₂O (0.1% FA), flow rate: 25 mL/min] to give (S)-7-{[5-(2,2-dimethyl-4-morpholinyl)-1-[(1S,2S)-2-methyl-1-(5-oxo-4,5-dihydro-1,2,4-oxadiazol-3-yl)cyclopropyl]-2-indolyl]carbonyl}-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (28.9 mg) as a white solid. m/z (ESI-pos) M+1=822.3; ¹H NMR (400 MHz, DMSO) δ 11.71 (s, 1H), 7.67-7.55 (m, 2H), 7.46-7.42 (m, 4H), 7.37-7.31 (m, 2H), 7.20-7.14 (m, 3H), 6.97-6.74 (m, 3H), 5.76-5.61 (m, 1H), 4.77-4.53 (m, 3H), 3.89-3.85 (m, 2H), 3.70-3.58 (m, 1H), 3.45-3.35 (m, 2H), 3.11-3.08 (m, 4H), 3.00-2.93 (m, 4H), 1.92-1.57 (m, 6H), 1.40-1.35 (m, 6H), 1.32-1.19 (m, 3H).

### Example 26—Synthesis of Compound 26

### Synthetic Scheme

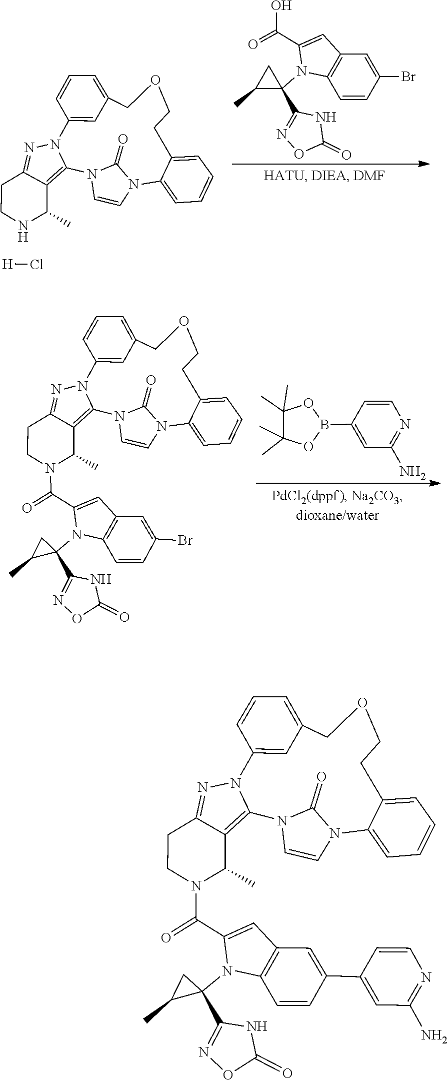

### Procedure for Synthesis of Compound 26

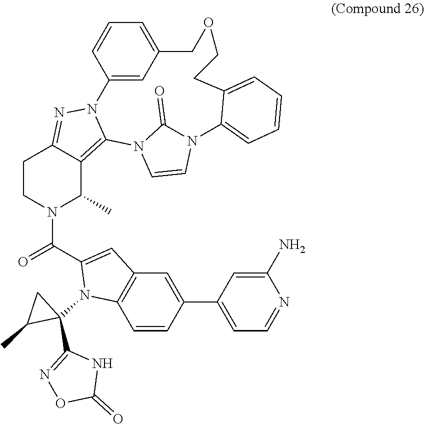

### (S)-7-{[5-(2-amino-4-pyridyl)-1-[(1S,2S)-2-methyl-1-(5-oxo-4,5-dihydro-1,2,4-oxadiazol-3-yl)cyclopropyl]-2-indolyl]carbonyl}-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: A solution of(S)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (480 mg, 1.03 mmol), 5-bromo-1-[(1S,2S)-2-methyl-1-(5-oxo-4,5-dihydro-1,2,4-oxadiazol-3-yl)cyclopropyl]-2-indolecarboxylic acid (469 mg, 1.24 mmol), HATU (590 mg, 1.55 mmol), and DIEA (541 μL, 3.1 mmol) in 1 mL of DMF was stirred for 1 hour at 50° C. The solution was then taken up in ethyl acetate and washed with water (3×). The organics were concentrated and purified by silica-gel chromatography (10-95% EtOAc/heptanes) to give (S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (550 mg, 663 μmol). m/z (ESI-pos) M+1=787.4

Step B: A solution of(S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (10 mg, 12.7 μmol), PdCl₂(dppf) (1.04 mg, 1.27 μmol), 4-(4,4,5,5-tetramethyl-1,3,2-dioxaborolan-2-yl)pyridin-2-amine (5.6 mg, 25.4 μmol), and disodium carbonate (6.73 mg, 63.5 μmol) in 0.1 mL of dioxane was heated to 90° C. for 1 hour. The solution was then diluted with MeOH, filtered, and purified directly by reverse-phase chromatography (5-95% MeCN/water, 0.1% FA), and the product was concentrated to give (S)-7-{[5-(2-amino-4-pyridyl)-1-[(1S,2S)-2-methyl-1-(5-oxo-4,5-dihydro-1,2,4-oxadiazol-3-yl)cyclopropyl]-2-indolyl]carbonyl}-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (8 mg, 9.99 μmol) as a white solid. m/z (ESI-pos) M+1=801.6. 1H NMR (400 MHz, chloroform-D) δ 8.37 (s, 2H), 7.93 (s, 1H), 7.82 (d, J=6.9 Hz, 1H), 7.75 (d, J=8.6 Hz, 1H), 7.57 (d, J=8.4 Hz, 2H), 7.51-7.46 (m, 1H), 7.34 (d, J=2.9 Hz, 3H), 7.22-7.15 (m, 1H), 7.10-7.03 (m, 1H), 6.96 (d, J=6.4 Hz, 1H), 6.89 (s, 1H), 6.83 (d, J=7.8 Hz, 1H), 6.51-6.21 (m, 2H), 6.07-5.76 (m, 1H), 4.82-4.38 (m, 4H), 3.75-3.61 (m, 1H), 3.34 (h, J=11.0 Hz, 3H), 3.24-3.13 (m, 1H), 3.06 (td, J=12.1, 5.9 Hz, 1H), 2.97-2.83 (m, OH), 1.97 (t, J=6.4 Hz, 1H), 1.65 (d, J=6.7 Hz, 2H), 1.62-1.49 (m, 1H), 1.22 (d, J=6.0 Hz, 3H).

### Example 27—Synthesis of Compound 27

### Synthetic Scheme

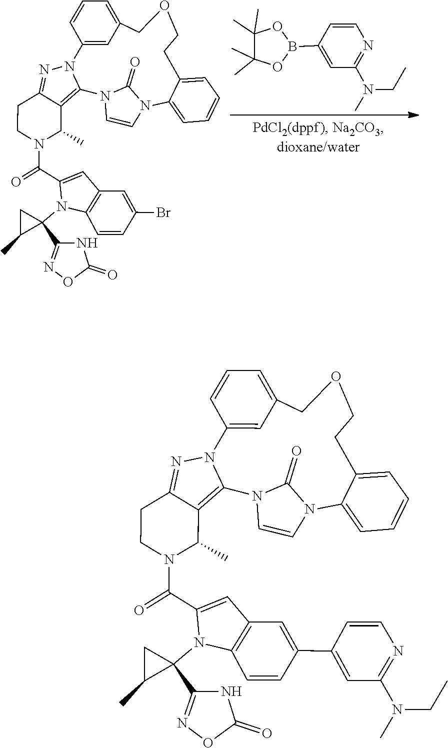

### Procedure for Synthesis of Compound 27

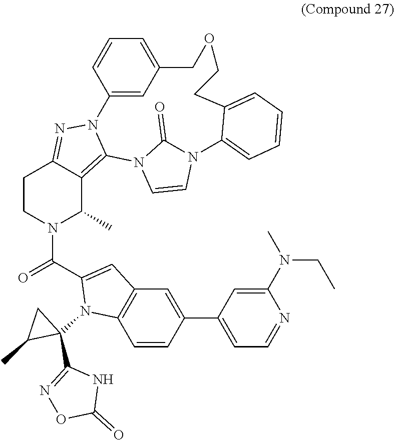

### (S)-8-methyl-7-({1-[(1S,2S)-2-methyl-1-(5-oxo-4,5-dihydro-1,2,4-oxadiazol-3-yl)cyclopropyl]-5-[2-(N-ethyl-N-methylamino)-4-pyridyl]-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: A solution of(S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (10 mg, 12.7 μmol) PdCl2(dppf) (1.04 mg, 1.27 μmol), N-ethyl-N-methyl-4-(4,4,5,5-tetramethyl-1,3,2-dioxaborolan-2-yl)pyridin-2-amine, and disodium carbonate (6.73 mg, 63.5 μmol) in 0.1 mL of dioxane was heated to 90° C. for 1 hour. The solution was then diluted with MeOH, filtered, and purified directly by reverse-phase chromatography (5-95% MeCN/water, 0.1% FA), and the product was concentrated to give (S)-8-methyl-7-({1-[(1S,2S)-2-methyl-1-(5-oxo-4,5-dihydro-1,2,4-oxadiazol-3-yl)cyclopropyl]-5-[2-(N-ethyl-N-methylamino)-4-pyridyl]-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (5 mg, 5.93 μmol). m/z (ESI-pos) M+1=843.7; ¹H NMR (400 MHz, chloroform-D) δ 8.26-8.17 (m, 1H), 7.95-7.89 (m, 1H), 7.73 (t, J=8.4 Hz, 1H), 7.60 (ddd, J=14.7, 7.7, 1.9 Hz, 2H), 7.48 (td, J=7.9, 3.2 Hz, 1H), 7.34 (d, J=3.0 Hz, 3H), 7.24-7.09 (m, 2H), 6.89-6.68 (m, 3H), 6.50-6.25 (m, 2H), 6.12-5.76 (m, 1H), 4.78-4.40 (m, 3H), 3.67 (q, J=6.9 Hz, 3H), 3.47-3.27 (m, 3H), 3.19-3.14 (m, 3H), 3.11-2.85 (m, 2H), 2.01-1.77 (m, 1H), 1.73-1.55 (m, 3H), 1.27-1.18 (m, 6H).

### Example 28—Synthesis of Compound 28

### Synthetic Scheme

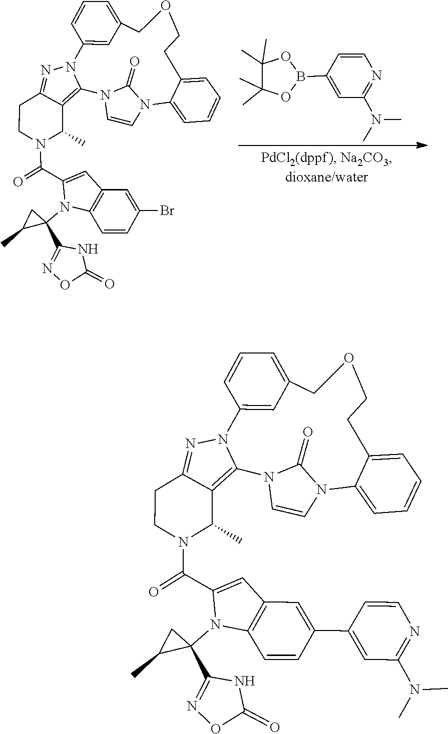

### Procedure for Synthesis of Compound 28

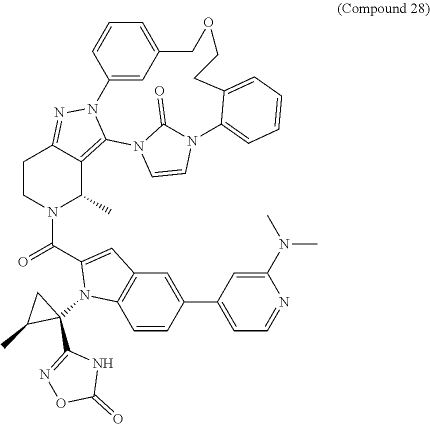

### (S)-7-({5-[2-(dimethylamino)-4-pyridyl]-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: A solution of(S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (15 mg, 19 μmol), PdCl2(dppf) (1.56 mg, 1.9 μmol), N,N-dimethyl[4-(dihydroxyboryl)-2-pyridyl]amine (6.32 mg, 38.1 μmol), and disodium carbonate (10.1 mg, 95.2 μmol) in 0.1 mL of dioxane was heated to 90° C. for 1 hour. The solution was then diluted with MeOH, filtered, and purified by reverse-phase chromatography (5-95% MeCN/water, 0.1% FA). The product was then concentrated to give (S)-7-({5-[2-(dimethylamino)-4-pyridyl]-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (8 mg, 9.65 μmol) as a white solid. m/z (ESI-pos) M+1=829.6; 1H NMR (400 MHz, chloroform-D) δ 8.23 (d, J=5.9 Hz, 3H), 7.93 (t, J=2.1 Hz, 1H), 7.77-7.69 (m, 1H), 7.67-7.45 (m, 3H), 7.33 (t, J=3.7 Hz, 3H), 7.23-7.16 (m, 1H), 6.94-6.85 (m, 1H), 6.85-6.74 (m, 2H), 6.51-6.22 (m, 2H), 6.11-5.76 (m, 1H), 4.90 (s, 4H), 4.71-4.36 (m, 3H), 3.68 (td, J=13.5, 4.3 Hz, 1H), 3.46-3.29 (m, 3H), 3.25-3.20 (m, 7H), 3.10-3.01 (m, 1H), 2.90 (td, J=11.7, 4.4 Hz, 1H), 2.01-1.86 (m, 1H), 1.81 (d, J=6.8 Hz, OH), 1.65 (d, J=6.7 Hz, 3H), 1.21 (d, J=6.0 Hz, 3H).

### Example 29—Synthesis of Compound 29

### Synthetic Scheme

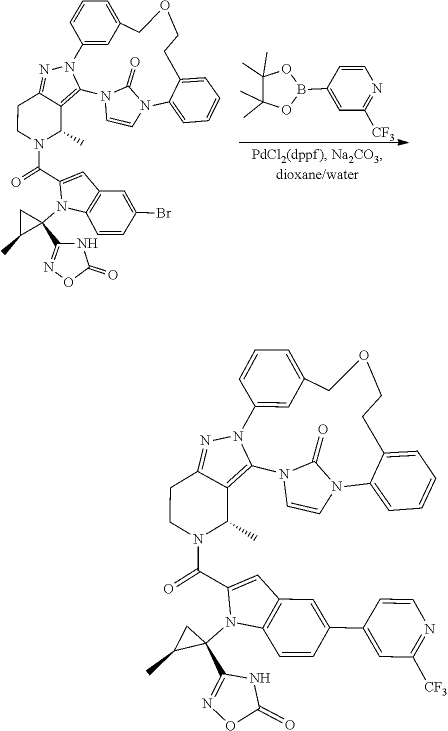

### Procedure for Synthesis of Compound 29

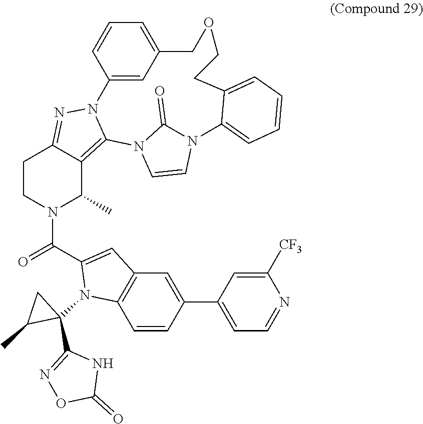

### (S)-8-methyl-7-({1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: A solution of(S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (12 mg, 15.2 μmol), PdCl2(dppf) (1.24 mg, 1.52 μmol), [2-(trifluoromethyl)-4-pyridyl]boranediol (5.82 mg, 30.5 μmol), and disodium carbonate (8.07 mg, 76.2 μmol) in 0.1 mL of dioxane was heated to 90° C. for 1 hour. The solution was then diluted with MeOH, filtered, and purified by reverse-phase chromatography (5-95% MeCN/water, 0.1% FA). The product was then concentrated to give (S)-8-methyl-7-({1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (5 mg, 5.86 μmol) as a white solid. m/z (ESI-pos) M+1=854.5; 1H NMR (400 MHz, chloroform-D) δ 11.22 (d, J=18.3 Hz, 1H), 8.80-8.70 (m, 1H), 8.04-7.97 (m, 1H), 7.93 (d, J=1.6 Hz, 1H), 7.79 (d, J=8.7 Hz, 1H), 7.72 (dd, J=5.1, 1.8 Hz, 1H), 7.65 (ddd, J=9.2, 7.4, 1.8 Hz, 1H), 7.60-7.55 (m, 1H), 7.48 (td, J=7.8, 3.0 Hz, 1H), 7.37-7.32 (m, 3H), 7.23-7.17 (m, 1H), 6.92-6.77 (m, 1H), 6.51-6.35 (m, 2H), 6.28-5.75 (m, 2H), 4.79-4.52 (m, 3H), 4.52-4.40 (m, 1H), 3.69 (td, J=13.2, 4.3 Hz, 1H), 3.35 (q, J=10.0 Hz, 3H), 3.23-3.00 (m, 2H), 2.95-2.73 (m, 1H), 2.02-1.89 (m, 1H), 1.68 (dd, J=13.4, 6.7 Hz, 3H), 1.22 (d, J=6.1 Hz, 3H).

### Example 30—Synthesis of Compound 30

### Synthetic Scheme

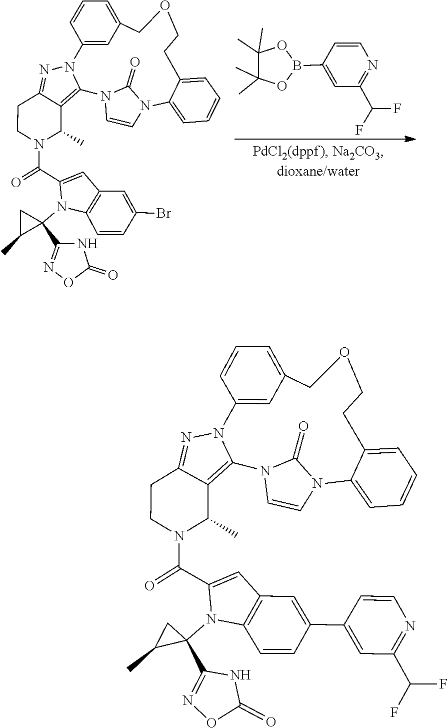

### Procedure for Synthesis of Compound 30

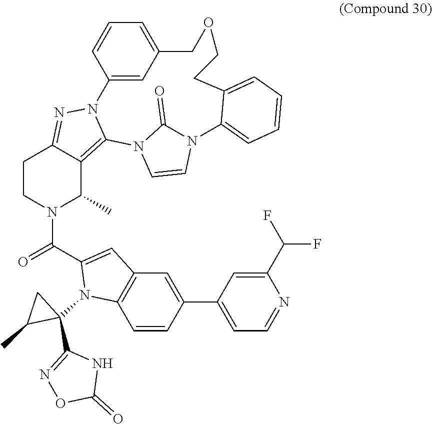

### (S)-7-({5-[2-(difluoromethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: A solution of(S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (15 mg, 19 μmol), PdCl2(dppf) (1.56 mg, 1.9 μmol), 2-[2-(difluoromethyl)-4-pyridyl]-4,4,5,5-tetramethyl-1,3,2-dioxaborolane (9.71 mg, 38.1 μmol), and disodium carbonate (10.1 mg, 95.2 μmol) in 1,4-dioxane (100 μL) was heated to 80° C. for 1 hour. The solution was then diluted with MeOH, filtered, and purified directly by reverse-phase chromatography (5-95% MeCN/water, 0.1% FA), and the product was concentrated to afford(S)-7-({5-[2-(difluoromethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (10 mg, 12 μmol) as a white solid. m/z (ESI-pos) M+1=836.6; 1H NMR (400 MHz, CHLOROFORM-D) δ 11.25 (s, 1H), 8.69 (d, J=5.2 Hz, 1H), 8.00 (d, J=1.9 Hz, 1H), 7.89 (s, 1H), 7.77 (t, J=8.6 Hz, 1H), 7.69-7.63 (m, 2H), 7.58 (d, J=8.3 Hz, 1H), 7.48 (t, J=7.8 Hz, 1H), 7.37-7.27 (m, 4H), 7.18 (dd, J=24.8, 8.4 Hz, 2H), 6.83 (d, J=5.4 Hz, 1H), 6.47 (d, J=3.1 Hz, 1H), 6.38 (d, J=3.1 Hz, 1H), 5.82 (q, J=6.6 Hz, 1H), 4.77-4.39 (m, 3H), 3.74-3.63 (m, 1H), 3.47-3.27 (m, 4H), 3.24-3.00 (m, 2H), 2.96-2.83 (m, 1H), 1.98 (t, J=6.0 Hz, 1H), 1.66 (d, J=6.7 Hz, 3H), 1.61 (d, J=6.5 Hz, 2H), 1.22 (d, J=6.0 Hz, 3H).

### Example 31—Synthesis of Compound 31

### Synthetic Scheme

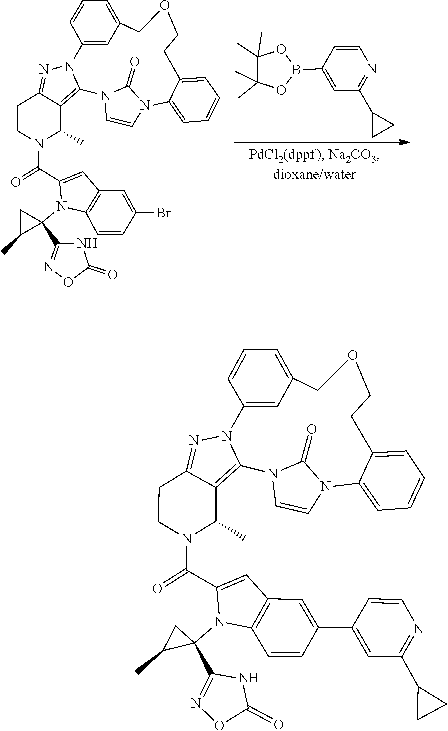

### Procedure for Synthesis of Compound 31

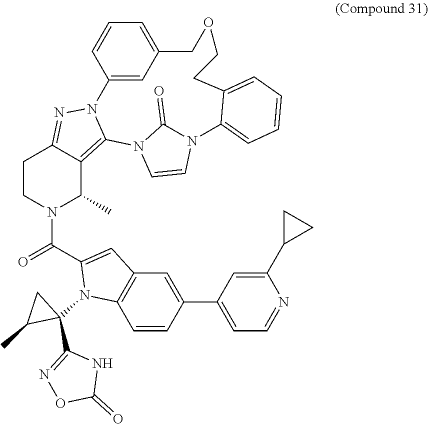

### ((S)-7-{[5-(2-cyclopropyl-4-pyridyl)-1-[(1S,2S)-2-methyl-1-(5-oxo-4,5-dihydro-1,2,4-oxadiazol-3-yl)cyclopropyl]-2-indolyl]carbonyl}-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: A solution of(S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (15 mg, 19 μmol), PdCl2(dppf) (1.56 mg, 1.9 μmol), (2-cyclopropyl-4-pyridyl)boranediol (6.21 mg, 38.1 μmol), and disodium carbonate (10.1 mg, 95.2 μmol) in 1,4-dioxane (100 μL) was heated to 90° C. for 1 hour. The solution was then diluted with MeOH, filtered, and purified directly by reverse-phase chromatography (5-95% MeCN/water, 0.1% FA), and the product was concentrated to afford(S)-7-{[5-(2-cyclopropyl-4-pyridyl)-1-[(1S,2S)-2-methyl-1-(5-oxo-4,5-dihydro-1,2,4-oxadiazol-3-yl)cyclopropyl]-2-indolyl]carbonyl}-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (11 mg, 70% yield) as a white solid. m/z (ESI-pos) M+1=826.6; 1H NMR (400 MHz, chloroform-D) δ 11.27 (s, 1H), 8.48 (d, J=5.1 Hz, 1H), 7.93 (d, J=1.7 Hz, 1H), 7.74 (d, J=8.7 Hz, 1H), 7.64 (dd, J=8.7, 1.7 Hz, 1H), 7.58 (d, J=7.9 Hz, 1H), 7.48 (t, J=7.8 Hz, 1H), 7.37 (s, 1H), 7.34 (d, J=3.3 Hz, 4H), 7.30-7.28 (m, 1H), 7.20-7.16 (m, 1H), 6.82 (s, 1H), 6.47 (d, J=3.1 Hz, 1H), 6.38 (d, J=3.1 Hz, 1H), 5.82 (q, J=6.6 Hz, 1H), 4.69-4.42 (m, 3H), 3.72-3.64 (m, 1H), 3.41-3.30 (m, 3H), 3.23-3.01 (m, 2H), 2.95-2.87 (m, 1H), 2.13-2.07 (m, 1H), 1.98-1.95 (m, 1H), 1.83 (d, J=6.7 Hz, 1H), 1.66 (d, J=6.7 Hz, 3H), 1.22 (d, J=5.9 Hz, 3H), 1.10-0.99 (m, 4H)

### Example 32—Synthesis of Compound 32

### Synthetic Scheme

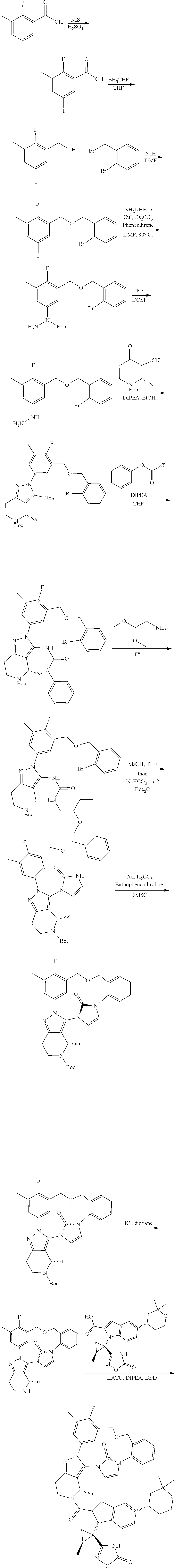

### Procedure for Synthesis of Compound 32

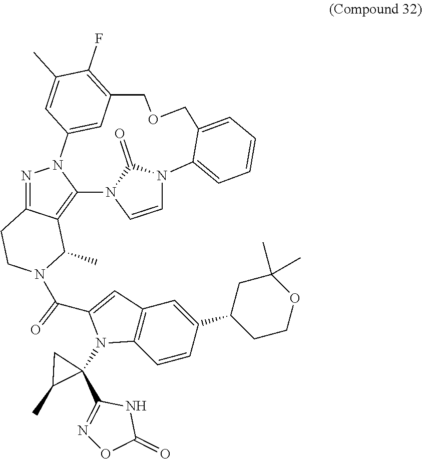

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: 2-fluoro-3-toluic acid (15 g, 97.3 mmol) and conc. aqueous sulfuric acid (75 mL) were added into the reaction flask and cooled to 0° C., 1-iodo-2,5-pyrrolidinedione (21.9 g, 97.3 mmol) was added portion-wise. After the addition was complete, the reaction temperature was naturally raised to room temperature and stirred for 2 h. Upon completion, the reaction was cooled in an ice bath and diluted with water, the mixture was filtered, rinsing with aqueous sodium dithionate solution (~500 mg in 200 mL water), then twice with water. The filter cake was dried in a vacuum oven overnight to afford 2-fluoro-5-iodo-3-toluic acid (26.1 g, 93.1 mmol). ¹H NMR (400 MHz, CHLOROFORM-D) δ 11.41 (s, 1H), 8.12 (dd, J=6.3, 2.4 Hz, 1H), 7.73 (ddd, J=6.3, 2.4, 0.9 Hz, 1H), 2.28 (d, J=2.4 Hz, 3H).

Step B: 1M Borane in THF (12.9 g, 150 mmol) was added dropwise to a stirred solution of 2-fluoro-5-iodo-3-toluic acid (21 g, 75 mmol) in THF (75 mL) cooled to 0° C. The resulting mixture was warmed to room temperature and stirred for an additional 3 h. The mixture was poured into a sat. aq. ammonium chloride solution and extracted with ethyl acetate. The combined organic phases were washed with sat. aq. sodium bicarbonate, brine, dried over Na₂SO₄ and concentrated to afford (2-fluoro-5-iodo-3-tolyl)methanol (19.9 g, 75 mmol) which was used in the next step without purification. ¹H NMR (400 MHz, CHLOROFORM-D) δ 7.56 (ddq, J=6.4, 2.5, 0.7 Hz, 1H), 7.48-7.41 (m, 1H), 4.69 (d, J=4.5 Hz, 2H), 2.22 (dt, J=2.1, 0.7 Hz, 3H), 1.74 (d, J=6.3 Hz, 1H).

Step C: To a solution of (2-fluoro-5-iodo-3-tolyl)methanol (970 mg, 3.65 mmol) and o-bromo(bromomethyl)benzene (911 mg, 3.65 mmol) in DMF (7.29 mL) was added sodium hydride 60% w/w (153 mg, 3.83 mmol) at 0° C. The reaction was stirred at 0° C. for 30 min and then at ambient temperature for 30 min. The reaction mixture was quenched with sat. aq. NH₄Cl, diluted with ethyl acetate, and washed with brine (2×). The organic layer was dried over Na₂SO₄, filtered and concentrated. The crude product was purified by silica gel column chromatography (0-10% EtOAc/heptane) to give 3-{[(o-bromophenyl)methoxy]methyl}-2-fluoro-5-iodotoluene (1.04 g, 2.4 mmol) as a yellow oil.

Step D: A solution of 3-{[(o-bromophenyl)methoxy]methyl}-2-fluoro-5-iodotoluene (1.04 g, 2.4 mmol), tert-butyl carbazate (413 mg, 3.12 mmol), dicaesium carbonate (1.1 g, 3.36 mmol), copper iodide (22.9 mg, 120 μmol), and 4,5-diazaphenanthrene (86.6 mg, 480 μmol) in DMF (2.4 mL) was sparged with Ar for 5 min and heated to 80° C. for 3 h. Upon cooling to rt, the solution was taken up in EtOAc, filtered through Celite, and washed with brine (3×). The organic phase was dried over Na₂SO₄, filtered, and concentrated. The crude product was purified by silica gel chromatography (5-30% EtOAc/heptane) to give tert-butyl 2-(m-{[2-(2-bromo-4,5-difluorophenyl) ethoxy]methyl}phenyl) carbazate (347 mg, 759 μmol) as a yellow oil. m/z (ESI-pos) M+1=383.1 and 385.1 (loss of tBu).

Step E: To a solution of tert-butyl 2-(3-{[(o-bromophenyl)methoxy]methyl}-4-fluoro-5-tolyl)carbazate (0.8 g, 1.82 mmol) in DCM (6.07 mL), trifluoroacetic acid (2.09 mL, 27.3 mmol) was added at ambient temperature. The reaction was stirred for 3 h. The reaction was then quenched with sat. aq. Na₂CO₃ (~20 mL) and extracted with DCM (1×), then washed with water (2×) and brine (1×). The combined aqueous layers were then extracted with 20% IPA/CHCl₃ (2×) and the combined organic layers were dried over Na₂SO₄, filtered, and concentrated to provide (3-{[(o-bromophenyl)methoxy]methyl}-4-fluoro-5-tolyl)hydrazine (618 mg, 1.82 mmol) as a yellow oil which was used without purification in the following step. m/z (ESI-pos) M+1=338.1 and 340.1.

Step F: To a solution of crude (3-{[(o-bromophenyl)methoxy]methyl}-4-fluoro-5-tolyl) hydrazine (618 mg, 1.82 mmol) in EtOH (7.29 mL) was added N-ethyldiisopropylamine (317 μL, 1.82 mmol) then tert-butyl(2S)-3-cyano-2-methyl-4-oxo-1-piperidinecarboxylate (521 mg, 2.19 mmol) at ambient temperature. The mixture was then stirred at 80° C. for 20 h. After completion, the reaction mixture was cooled and the solvent was evaporated under vacuo. The crude material was purified by column chromatography on silica gel (10-60% EtOAc/heptane) to give tert-butyl (S)-3-amino-2-(3-{[(o-bromophenyl)methoxy]methyl}-4-fluoro-5-tolyl)-4-methyl-1,2,5-triaza-2,4,6,7-tetrahydro-5H-indene-5-carboxylate (554 mg, 990 μmol) as a yellow solid over 2 steps. m/z (ESI-pos) M+1=559.3 and 561.3.

Step G: To a solution of tert-butyl (S)-3-amino-2-(3-{[(o-bromophenyl)methoxy]methyl}-4-fluoro-5-tolyl)-4-methyl-1,2,5-triaza-2,4,6,7-tetrahydro-5H-indene-5-carboxylate (554 mg, 990 μmol) in THF (5 mL) was added N-ethyldiisopropylamine (517 μL, 2.97 mmol) followed by benzenyl chloroformate (248 μL, 1.98 mmol). The reaction mixture was stirred at room temperature for 30 min. The reaction mixture was directly concentrated under vacuo to give the crude tert-butyl (S)-2-(3-{[(0-bromophenyl)methoxy]methyl}-4-fluoro-5-tolyl)-4-methyl-3-[phenyl(oxycarbonylamino)]-1,2,5-triaza-2,4,6,7-tetrahydro-5H-indene-5-carboxylate (673 mg, 990 μmol) as a yellow solid, which was used directly in the next step without any further purification. m/z (ESI-pos) M+1=679.3 and 681.3.

Step H: Crude tert-butyl (S)-2-(3-{[(o-bromophenyl)methoxy]methyl}-4-fluoro-5-tolyl)-4-methyl-3-[phenyl(oxycarbonylamino)]-1,2,5-triaza-2,4,6,7-tetrahydro-5H-indene-5-carboxylate (673 mg, 990 μmol) was dissolved in pyridine (3.3 mL) and then 2,2-dimethoxyethylamine (162 μL, 1.49 mmol) was added. The mixture was stirred at room temperature for 30 min and then directly concentrated under vacuo to give the crude residue which was purified by column chromatography (5-80% EtOAc/heptane) to give tert-butyl (S)-2-(3-{[(o-bromophenyl)methoxy]methyl}-4-fluoro-5-tolyl)-3-[3-(2,2-dimethoxyethyl)ureido]-4-methyl-1,2,5-triaza-2,4,6,7-tetrahydro-5H-indene-5-carboxylate (464 mg, 672 μmol) as a white solid over 2 steps. m/z (ESI-pos) M+1=689.3 and 691.3.

Step I: To a solution of tert-butyl (S)-2-(3-{[(2-bromo-4-fluorophenyl)methoxy]methyl}-4-fluoro-5-tolyl)-3-[3-(2,2-dimethoxyethyl)ureido]-4-methyl-1,2,5-triaza-2,4,6,7-tetrahydro-5H-indene-5-carboxylate (464 mg, 655 μmol) in THF (3.3 mL) was added methanesulfonic acid (128 μL, 1.96 mmol). The mixture was stirred at 55° C. for 1 h and then cooled to ambient temperature. Next, saturated aqueous NaHCO₃ was added until the solution was pH 8 and then tert-butoxycarbonyl-tert-butyl carbonate (157 mg, 720 μmol) was added, and the resulting mixture was stirred at room temperature for 30 min. Upon completion, the reaction mixture was diluted with water and extracted with ethyl acetate (2×). The combined organic layers were washed with brine and dried over anhydrous Na₂SO₄, filtered and concentrated under vacuo. The crude compound was purified by column chromatography (5-90% EtOAc/heptane) to give tert-butyl (S)-2-(3-{[(o-bromophenyl)methoxy]methyl}-4-fluoro-5-tolyl)-4-methyl-3-(2-oxo-2,3-dihydro-1H-imidazol-1-yl)-1,2,5-triaza-2,4,6,7-tetrahydro-5H-indene-5-carboxylate (340 mg, 543 μmol) as an off-white solid. m/z (ESI-pos) M+1=626.3 and 628.3.

Step J: A solution of tert-butyl (S)-2-(3-{[(o-bromophenyl)methoxy]methyl}-4-fluoro-5-tolyl)-4-methyl-3-(2-oxo-2,3-dihydro-1H-imidazol-1-yl)-1,2,5-triaza-2,4,6,7-tetrahydro-5H-indene-5-carboxylate (340 mg, 543 μmol), copper iodide (77.5 mg, 407 μmol), bathophenanthroline (180 mg, 543 μmol), and dipotassium carbonate (187 mg, 1.36 mmol) in DMSO (18.1 mL) was sparged with Ar for 5 minutes and then heated to 130° C. overnight for 21 h. The reaction was diluted with EtOAc and sat. aq. NH₄Cl and then filtered through a pad of celite. The filtrate was partitioned, and the organic phase was washed with brine, dried with sodium sulfate, filtered, concentrated under vacuo. The crude material was purified by silica gel chromatography (5-50% EtOAc/heptane), which provided separation of the atropisomers. The major atropisomer product was tert-butyl (8S,10M)-25-fluoro-8,26-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (100 mg, 183 μmol). m/z (ESI-pos) M+1=546.4.

Step K: 4.0M HC in dioxane (2 mL) was added to a vial containing tert-butyl (8S,10M)-25-fluoro-8,26-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (27.1 mg, 49.7 μmol) and the solution stirred at ambient temperature for 20 min. Upon completion, the solvent was evaporated to give a crude white residue which was used in the next step without purification. m/z (ESI-pos) M+1=446.3

Step L: To a solution of crude (8S,10M)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (23.9 mg, 49.6 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (26.5 mg, 64.5 μmol) in DMF (1 mL) was added N-ethyldiisopropylamine (43.2 μL, 248 μmol) and HATU (37.7 mg, 99.2 μmol). The mixture stirred at ambient temperature overnight. The reaction mixture was directly loaded onto the reverse phase column and purified by Prep HPLC (Isco ACCQ Prep, Gemini column NX—C18, 250×21 mm, 20 ml/min, 45 minute separation, 20 to 80% H₂O/ACN with 0.1% Formic acid buffer), pure fractions were collected and lyophilized to give (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (33.6 mg). m/z (ESI-pos) M+1=839.6. ¹H NMR (400 MHz, CHLOROFORM-D) δ 11.29 (d, J=18.4 Hz, 1H), 7.62-7.27 (m, 8H), 7.14-7.01 (m, 1H), 6.79-6.68 (m, 1H), 6.47-5.86 (m, 2H), 5.85-5.19 (m, 1H), 5.00-4.41 (m, 3H), 4.39-4.09 (m, 2H), 3.98-3.77 (m, 1H), 3.70-3.34 (m, 1H), 3.24-2.96 (m, 3H), 2.36-2.29 (m, 3H), 1.87 (dt, J=25.3, 6.1 Hz, 1H), 1.81-1.59 (m, 7H), 1.59-1.48 (m, 3H), 1.33 (s, 3H), 1.27 (d, J=3.2 Hz, 3H), 1.16 (dd, J=29.5, 6.0 Hz, 3H).

### Example 33—Synthesis of Compound 33

### Synthetic Scheme

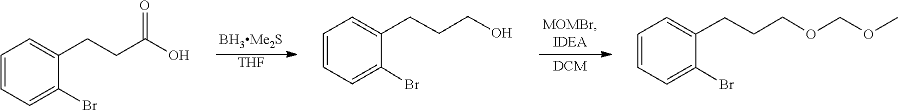

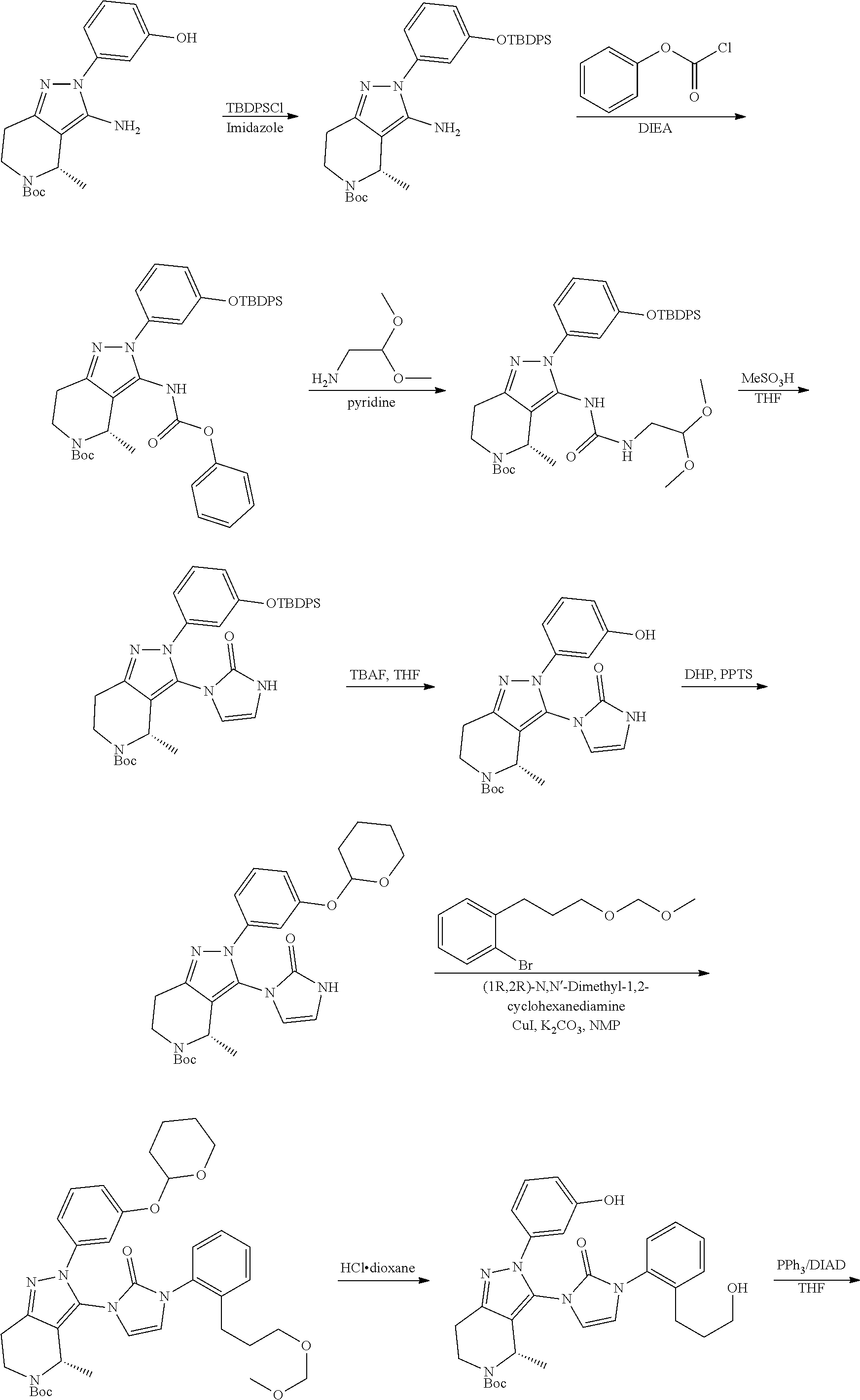

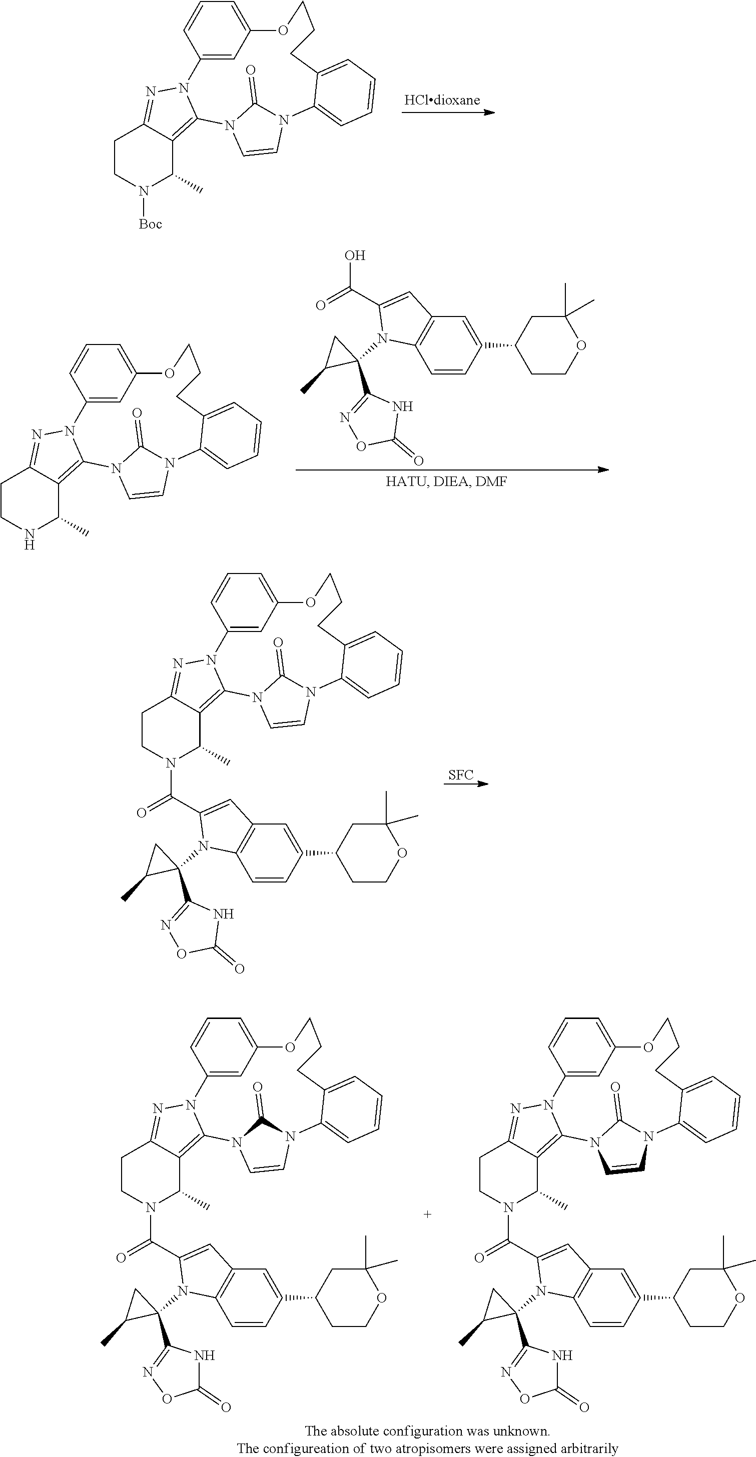

### Procedure for Synthesis of Compound 33

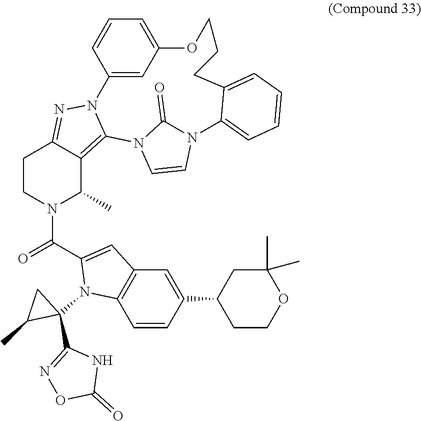

### (S)-7-({5-[(S)-2,2-dimethyltetrahydro-4H-pyran-4-yl]-1-[(2S)-2-methyl-1-(5-oxo-4,5-dihydro-1,2,4-oxadiazol-3-yl)cyclopropyl]-2-indolyl}carbonyl)-8-methyl-24-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a solution of 3-(o-bromophenyl) propionic acid (2 g, 8.73 mmol) in THF (30 mL) at 0° C. was added BH₃·Me₂S complex (13.1 mL, 13.1 mmol, 1.0M in THF). The mixture was allowed to warm to ambient temperature and stirred for 16 hours. The reaction was quenched by addition of H₂O and extracted with EtOAc (2×). The combined EtOAc layers were washed with brine, dried over Na₂SO₄, filtered and concentrated. The crude product was purified by column chromatography (0-25% EtOAc in PE) to give 3-(0-bromophenyl)-1-propanol (1.7 g, 7.11 mmol, 90% yield) as a white solid. ¹H NMR (400 MHz, CDCl₃) δ 7.52 (d, J=8.0 Hz, 1H), 7.24-7.22 (m, 2H), 7.07-7.03 (m, 1H), 3.69 (t, J=6.4 Hz, 2H), 2.85-2.81 (m, 2H), 1.92-1.85 (m, 2H).

Step B: To a solution of 3-(o-bromophenyl)-1-propanol (1.7 g, 7.11 mmol) in DCM (20 mL) at 0° C. was added DIEA (1.84 g, 14.2 mmol) and MOMBr (978 mg, 7.82 mmol). The mixture was allowed to warm to ambient temperature and stirred for 2 hours. The reaction mixture was quenched by addition of H₂O and extracted with DCM (2×). The combined DCM layers were washed with brine, dried over Na₂SO₄, filtered and concentrated in vacuo. The crude product was purified by column chromatography (0-20% EA in PE) to give 1-bromo-2-(3-(methoxymethoxy)propyl)benzene (2.0 g, 6.95 mmol) as a colorless oil. ¹H NMR (400 MHz, CDCl₃) δ 7.53 (d, J=7.6 Hz, 1H), 7.26-7.22 (m, 2H), 7.07-7.03 (m, 1H), 4.65 (s, 2H), 3.58 (t, J=6.4 Hz, 2H), 3.39 (s, 3H), 2.85-2.81 (m, 2H), 1.96-1.89 (m, 2H).

Step C: To a solution of tert-butyl (S)-3-amino-2-(m-hydroxyphenyl)-4-methyl-1,2,5-triaza-2,4,6,7-tetrahydro-5H-indene-5-carboxylate (1 g, 2.61 mmol) in DCM (20 mL) at 0° C. were added imidazole (356 mg, 5.23 mmol) and tert-butyl(chloro)bis(phenyl)silane (790 mg, 2.87 mmol). The mixture was allowed to warm to ambient temperature and stirred for 2 hours. The reaction was quenched with H₂O and extracted with DCM (2×). The combined organics were dried over Na₂SO₄, filtered and concentrated under reduced pressure. The crude product was purified by column chromatography (0-25% EtOAc in PE) to give tert-butyl(S)-3-amino-2-{m-[tert-butylbis(phenyl)siloxy]phenyl}-4-methyl-1,2,5-triaza-2,4,6,7-tetrahydro-5H-indene-5-carboxylate (1.6 g, 2.47 mmol) as a yellow solid. m/z (ESI-pos) M+1=583.3

Step D: To a solution of tert-butyl (S)-3-amino-2-{m-[tert-butylbis(phenyl)siloxy]phenyl}-4-methyl-1,2,5-triaza-2,4,6,7-tetrahydro-5H-indene-5-carboxylate (1.6 g, 2.47 mmol) in THF (20 mL) at 0° C. were added DIEA (958 mg, 7.41 mmol) and benzenyl chloroformate (774 mg, 4.94 mmol). The mixture was allowed to warm to ambient temperature and stirred for 2 hours, then directly concentrated under vacuum to get crude tert-butyl (S)-2-(3-((tert-butyldiphenylsilyl)oxy)phenyl)-4-methyl-3-((phenoxy carbonyl)amino)-2,4,6,7-tetrahydro-5H-pyrazolo[4,3-c]pyridine-5-carboxylate (2.9 g) as a light yellow solid, which was directly used in next step without further purification. m/z (ESI-pos) M+1=703.3.

Step E: To a solution of tert-butyl (S)-2-{m-[tert-butylbis(phenyl)siloxy]phenyl}-4-methyl-3-[phenyl(oxycarbonylamino)]-1,2,5-triaza-2,4,6,7-tetrahydro-5H-indene-5 carboxylate (2.9 g, 2.48 mmol) in pyridine (20 mL) was added 2,2-dimethoxyethylamine (781 mg, 7.43 mmol) and the mixture was stirred at 20° C. for 2 hours. The reaction mixture was concentrated and the crude product was purified by column chromatography (0-50% EtOAc in PE) to give tert-butyl (S)-2-{m-[tert-butylbis(phenyl)siloxy]phenyl}-3-[3-(2,2-dimethoxyethyl)ureido]-4-methyl-1,2,5-triaza-2,4,6,7-tetrahydro-5H-indene-5 carboxylate (1.6 g, 2.02 mmol, 81% yield over 2 steps) as a white solid. m/z (ESI-pos) M+1=714.3

Step F: To a solution of tert-butyl (S)-2-{m-[tert-butylbis(phenyl)siloxy]phenyl}-3-[3-(2,2-dimethoxyethyl)ureido]-4-methyl-1,2,5-triaza-2,4,6,7-tetrahydro-5H-indene carboxylate (1.6 g, 2.02 mmol) in THF (30 mL) was added methanesulfonic acid (194 mg, 2.02 mmol) and the mixture solution was stirred at 60° C. for 1 hour. The reaction solution was quenched with H₂O and extracted with EtOAc (2×). The combined organic layers were dried over anhydrous Na₂SO₄, filtered and concentrated. The crude product was purified by column chromatography (0-100% EtOAc in PE) to give tert-butyl (S)-2-{m-[tert-butylbis(phenyl)siloxy]phenyl}-4-methyl-3-(2-oxo-2,3-dihydro-1H-imidazol-1-yl)-1,2,5-triaza-2,4,6,7-tetrahydro-5H-indene-5-carboxylate (1.2 g, 1.66 mmol, 82% yield) as a white solid. m/z (ESI-pos) M+1=650.3.

Step G: To a solution of tert-butyl (S)-2-(3-((tert-butyldiphenylsilyl)oxy)phenyl)-4-methyl-3-(2-oxo-2,3-dihydro-1H-imidazol-1-yl)-2,4,6,7-tetrahydro-5H-pyrazolo[4,3-c]pyridine-5-carboxylate (980 mg, 1.36 mmol) in THF (12.6 mL) was added TBAF (1.0 M in THF, 1.62 mL, 2.04 mmol) dropwise at 0° C. After addition, the mixture was allowed to warm to ambient temperature and stirred for 2 hours. H₂O was added into the mixture to quench the reaction and extracted with EtOAc (2×). The combined organic layers were dried over anhydrous Na₂SO₄, filtered and concentrated under reduced pressure. The crude product was purified by column chromatography (0-100% EtOAc in PE) to give tert-butyl (S)-2-(3-hydroxyphenyl)-4-methyl-3-(2-oxo-2,3-dihydro-1H-imidazol-1-yl)-2,4,6,7-tetrahydro-5H-pyrazolo[4,3-c]pyridine-5-carboxylate (560 mg, 1.22 mmol, 90% yield) as a white solid. m/z (ESI-pos) M+1=412.2.

Step H: To a solution of tert-butyl (S)-2-(3-hydroxyphenyl)-4-methyl-3-(2-oxo-2,3-dihydro-1H-imidazol-1-yl)-2,4,6,7-tetrahydro-5H-pyrazolo[4,3-c]pyridine-5-carboxylate (560 mg, 1.22 mmol) in DCM (25 mL) was added 3,4-dihydro-2H-pyran (515 mg, 6.12 mmol) and 1-pyridinium p-toluenesulfonate (61.6 mg, 245 μmol). The mixture was stirred at ambient temperature for 16 hours. The reaction mixture was diluted with H₂O and extracted with EtOAc (2×). The combined organic layers were dried over anhydrous Na₂SO₄, filtered and concentrated under reduced pressure. The crude product was purified by column chromatography (0-50% EtOAc in PE) to give tert-butyl (4S)-4-methyl-3-(2-oxo-2,3-dihydro-1H-imidazol-1-yl)-2-(3-((tetrahydro-2H-pyran-2-yl)oxy)phenyl)-2,4,6,7-tetrahydro-5H-pyrazolo[4,3 c]pyridine-5-carboxylate (590 mg, 1.07 mmol) as a white solid. m/z (ESI-pos) M+1=496.2.

Step I: To a solution of tert-butyl (4S)-4-methyl-3-(2-oxo-2,3-dihydro-1H-imidazol-1-yl)-2-(3-((tetrahydro-2H-pyran-2-yl)oxy)phenyl)-2,4,6,7-tetrahydro-5H-pyrazolo[4,3-c]pyridine-5-carboxylate (590 mg, 1.07 mmol) in NMP (30 mL) were added 1-bromo-2-(3-(methoxymethoxy)propyl)benzene (694 mg, 2.68 mmol), copper iodide (102 mg, 536 μmol), (1R,2R)-1,2-bis(methylamino)cyclohexane (152 mg, 1.07 mmol) and dipotassium carbonate (444 mg, 3.21 mmol) under N₂ atmosphere. The mixture was stirred at 100° C. for 16 hours. The reaction mixture was cooled to ambient temperature and diluted with H₂O then extracted with EtOAc (2×). The combined EtOAc layers were washed with LiCl aqueous (2×), then dried over Na₂SO₄, filtered and concentrated in vacuo. The crude product was purified by column chromatography (0-50% EtOAc in PE) to give tert-butyl (4S)-3-(3-(2-(3-(methoxymethoxy)propyl)phenyl)-2-oxo-2,3-dihydro-1H-imidazol-1-yl)-4-methyl-2-(3-((tetrahydro-2H-pyran-2-yl)oxy)phenyl)-2,4,6,7-tetrahydro-5H-pyrazolo[4,3-c]pyridine-5-carboxylate (0.5 g, 668 μmol) as a white solid. m/z (ESI-pos) M+1=674.3.

Step J: To a solution of tert-butyl (4S)-3-(3-(2-(3-(methoxymethoxy)propyl)phenyl)-2-oxo-2,3-dihydro-1H-imidazol-1-yl)-4-methyl-2-(3-((tetrahydro-2H-pyran-2-yl)oxy)phenyl)-2,4,6,7-tetrahydro-5H-pyrazolo[4,3-c]pyridine-5-carboxylate (0.5 g, 668 μmol) in DCM (5 mL) was added HC/1,4-dioxane (4M, 5 mL) at 0° C. The mixture was then stirred at ambient temperature for 1 hour. The mixture was concentrated under reduced pressure and aqueous K₃PO₄ was added to the residue to adjusted pH to about 9, and then Boc₂O (175 mg, 0.80 mmol) was added. The resulting mixture was stirred at ambient temperature for 2 hrs. Aqueous citric acid was added and the mixture was extracted with EtOAc (3×). The combined organic layers were dried over anhydrous Na₂SO₄, filtered and concentrated. The crude product was purified by column chromatography (0-100% EtOAc in PE) to give tert-butyl(S)-2-(3-hydroxyphenyl)-3-(3-(2-(3-hydroxypropyl)phenyl)-2-oxo-2,3-dihydro-1H-imidazol-1-yl)-4-methyl-2,4,6,7-tetrahydro-5H-pyrazolo[4,3-c]pyridine-5-carboxylate (275 mg, 454 μmol) as a white solid. m/z (ESI-pos) M+1=546.3.

Step K: To a solution of tert-butyl (S)-2-(3-hydroxyphenyl)-3-(3-(2-(3-hydroxypropyl)phenyl)-2-oxo-2,3-dihydro-1H-imidazol-1-yl)-4-methyl-2,4,6,7-tetrahydro-5H-pyrazolo[4,3-c]pyridine-5-carboxylate (275 mg, 454 μmol) and triphenylphosphine (297 mg, 1.13 mmol) in THF (25 mL) at 0° C. was added DIAD (229 mg, 1.13 mmol) dropwise under N₂ atmosphere. The resulting mixture was then stirred at 60° C. for 2 hours. The reaction mixture was cooled and quenched with H₂O and extracted with EtOAc (2×). The combined EtOAc layers were washed with brine then dried over Na₂SO₄, filtered and concentrated in vacuo. The crude product was purified by column chromatography (0-40% EtOAc in PE) to give tert-butyl (S)-8-methyl-30-oxo-24-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (125 mg, 213 μmol) as a white solid. m/z (ESI-pos) M+1=528.3.

Step L: A solution of tert-butyl (S)-8-methyl-30-oxo-24-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (125 mg, 213 μmol) in HCl/1,4-dioxane (4M, 10 mL) was stirred at 20° C. for an hour. The mixture was directly concentrated under vacuum to give crude(S)-8-methyl-24-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (98 mg) as a white solid. m/z (ESI-pos) M+1=428.2

Step M: To a solution of 5-[(S)-2,2-dimethyltetrahydro-4H-pyran-4-yl]-1-[(2S)-2-methyl-1-(5-oxo-4,5-dihydro-1,2,4-oxadiazol-3-yl)cyclopropyl]-2-indolecarboxylic acid (84.9 mg, 206 μmol) in DMF (5 mL) were added DIEA (107 mg, 825 μmol) and HATU (118 mg, 309 μmol). The mixture was stirred at ambient temperature for 30 mins and then(S)-8-methyl-24-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (98 mg, 206 μmol) was added. The resulting mixture was stirred at 25° C. for 16 hours. H₂O was added and the mixture was extracted with EtOAc (2×). The combined EtOAc layers were washed with brine then dried over Na₂SO₄, filtered and concentrated in vacuo. The crude residue was purified by column chromatography (0-3% MeOH in DCM) to give (S)-7-({5-[(S)-2,2-dimethyltetrahydro-4H-pyran-4-yl]-1-[(2S)-2-methyl-1-(5-oxo-4,5-dihydro-1,2,4-oxadiazol-3-yl)cyclopropyl]-2-indolyl}carbonyl)-8-methyl-24-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (140 mg, 153 μmol) as a white solid. m/z (ESI-pos) M+1=821.3.

Step N: (S)-7-({5-[(S)-2,2-dimethyltetrahydro-4H-pyran-4-yl]-1-[(2S)-2-methyl-1-(5-oxo-4,5-dihydro-1,2,4-oxadiazol-3-yl)cyclopropyl]-2-indolyl}carbonyl)-8-methyl-24-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (140 mg, 153 μmol) was dissolved in MeOH (150 mL) and separated by chiral supercritical fluid chromatography (separation condition: Daicel CHIRALPAK IA-H, 250 mm×30 mm I.D., 10 μm; Mobile Phase: CO₂/MeOH[0.2% NH₃ (7M Solution in MeOH)]=45/55 at 120 g/min; Temp: 35° C.; Wavelength: 214 nm) to afford the crude Isomer 1 (45 mg), which was further purified by prep-HPLC [Waters MS-triggered Prep-LC with QDA detector, WELCH Xtimate C18 21.2*250 mm 10 μm, eluted with 75-100% ACN in H₂O (0.1% FA)] to give pure Isomer 1 (11.72 mg) and Isomer 2 (31.21 mg) as white solids (The absolute configuration was unknown, the configuration of two atropisomers were assigned arbitrarily).

Isomer 1: m/z (ESI-pos) M+1=821.3; ¹H NMR (400 MHz, DMSO) δ 11.56 (s, 1H), 7.51 (s, 1H), 7.46 (t, J=8.0 Hz, 1H), 7.43-7.39 (m, 5H), 7.29-7.24 (m, 2H), 7.01-6.99 (m, 1H), 6.88 (s, 1H), 6.81 (s, 1H), 6.67 (d, J=2.8 Hz, 1H), 6.64 (s, 1H), 5.64 (s, 1H), 4.43 (s, 1H), 4.29-4.22 (m, 1H), 4.14-4.08 (m, 1H), 3.75-3.72 (m, 2H), 3.60 (s, 1H), 3.00-2.88 (m, 3H), 2.65-2.57 (m, 1H), 1.72-1.61 (m, 9H), 1.57-1.51 (m, 3H), 1.29-1.27 (m, 4H), 1.20 (s, 6H).

Isomer 2: m/z (ESI-pos) M+1=821.3; ¹H NMR (400 MHz, DMSO) δ 11.49 (s, 1H), 7.48 (s, 1H), 7.41-7.35 (m, 4H), 7.30-7.20 (m, 3H), 7.21 (d, J=8.0 Hz, 1H), 6.96 (d, J=7.6 Hz, 1H), 6.86-6.71 (m, 4H), 5.61 (s, 1H), 4.40 (s, 1H), 4.17 (t, J=6.4 Hz, 2H), 3.73-3.71 (m, 2H), 3.51 (s, 1H), 3.02-2.98 (m, 1H), 2.89-2.81 (m, 2H), 2.66-2.60 (m, 1H), 1.74-1.50 (m, 9H), 1.45-1.42 (m, 3H), 1.28-1.25 (m, 4H), 1.19 (s, 6H).

### Example 34—Synthesis of Compound 34

### Synthetic Scheme

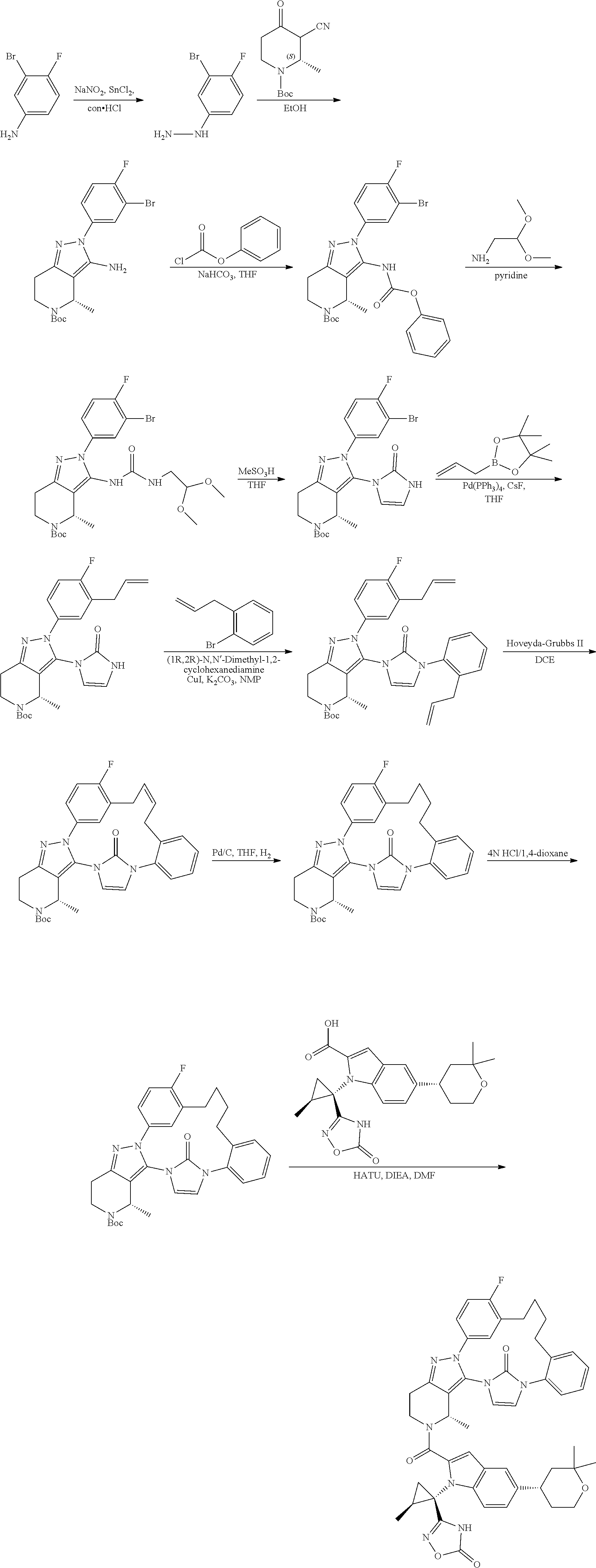

### Procedure for Synthesis of Compound 34

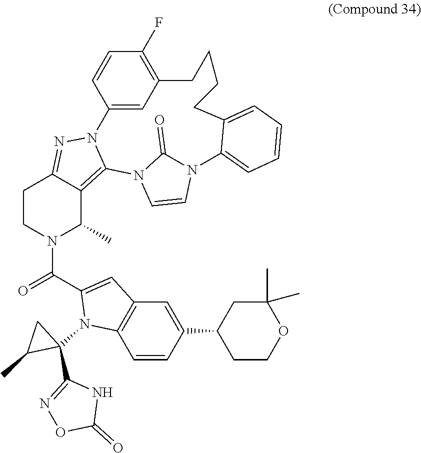

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: A solution of NaNO₂ (2.0 g, 28.9 mmol) in water (10 mL) was added dropwise to a mixture of 3-bromo-4-fluoroaniline (5.5 g, 28.9 mmol) in concentrated HCl (100 mL, 6 M) at 0° C. for 10 min. The resulting clear solution was stirred at 25° C. for 60 min. A solution of stannous chloride (11 g, 57.9 mmol) in concentrated HCl (10 mL) was added into the above mixture dropwise at 0° C. The mixture was stirred for 1 h at room temperature. The reaction mixture was diluted with water and adjusted the pH to about 10 by 5 Naq. NaOH, the suspension was filtered and the filtrate was extracted with ethyl acetate (3×). The combined organic layers were washed with brine solution and dried over anhydrous Na₂SO₄, filtered and concentrated. The crude compound was purified by column chromatography (0-100% EtOAc in PE) to give (3-bromo-4-fluorophenyl)hydrazine (5.4 g, 19.5 mmol, 72.8% yield) as a yellow solid. m/z (ESI-pos) M+1=205.1

Step B: To a mixture of (3-bromo-4-fluorophenyl)hydrazine (5.21 g, 25.4 mmol)_in ethanol (100 mL) was added tert-butyl(2S)-3-cyano-2-methyl-4-oxopiperidine-1-carboxylate (4.66 g, 19.58 mmol) at 25° C. The resulting mixture was stirred at 80° C. for 8 hr. The reaction mixture was cooled to room temperature, and diluted with ethyl acetate. The mixture was washed with water (3×) and the combined aqueous layers were extracted with ethyl acetate (2×). The combined organic layers were washed with brine, dried over anhydrous Na₂SO₄, filtered and the filtrate was evaporated under reduced pressure. The residue was purified by column chromatography (0-100% EtOAc in PE) to give tert-butyl (S)-3-amino-2-(3-bromo-4-fluorophenyl)-4-methyl-2,4,6,7-tetrahydro-5H-pyrazolo[4,3-c]pyridine-5-carboxylate (6.7 g, 11.8 mmol) as a yellow solid. m/z (ESI-pos) M+1=425.0

Step C: To a solution of tert-butyl (S)-3-amino-2-(3-bromo-4-fluorophenyl)-4-methyl-2,4,6,7-tetrahydro-5H-pyrazolo[4,3-c]pyridine-5-carboxylate (3000 mg, 7.05 mmol), sodium bicarbonate (1.77 g, 21.15 mmol) and tetrahydrofuran (60 mL) was added benzenyl chloroformate (2.21 g, 14.11 mmol). The reaction mixture was stirred at 25° C. for 2 h and then directly concentrated under vacuum to give crude tert-butyl (S)-2-(3-bromo-4-fluorophenyl)-4-methyl-3-((phenoxycarbonyl)amino)-2,4,6,7-tetrahydro-5H-pyrazolo[4,3-c]pyridine-5-carboxylate (4.1 g crude) as a yellow oil, which was used directly for next step without any further purification. m/z (ESI-pos) M+1=545.1.

Step D: The above obtained crude tert-butyl (S)-2-(3-bromo-4-fluorophenyl)-4-methyl-3-((phenoxycarbonyl)amino)-2,4,6,7-tetrahydro-5H-pyrazolo[4,3-c]pyridine-5-carboxylate (4.1 g, 7.05 mmol) was dissolved in pyridine (50 mL), to which 2,2-dimethoxyethylamine (2.22 g, 21.15 mmol) was added. The mixture was stirred at 25° C. for 3 h and then concentrated under vacuum. The residue was purified by column chromatography (0-60% EtOAc in PE) to give tert-butyl (S)-2-(3-bromo-4-fluorophenyl)-3-(3-(2,2-dimethoxyethyl)ureido)-4-methyl-2,4,6,7-tetrahydro-5H-pyrazolo[4,3-c]pyridine-5-carboxylate (2.98 g, 4.82 mmol) as a yellow solid. m/z (ESI-pos) M+1=556.3

Step E: A solution of tert-butyl (S)-2-(3-bromo-4-fluorophenyl)-3-(3-(2,2-dimethoxyethyl)ureido)-4-methyl-2,4,6,7-tetrahydro-5H-pyrazolo[4,3-c]pyridine-5-carboxylate (2.98 g, 5.36 mmol) in tetrahydrofuran (100 mL) was treated with methanesulfonic acid (669 mg, 6.96 mmol). The mixture was stirred at 60° C. for 2 h and then cooled to ambient temperature. The mixture was diluted with ethyl acetate and basified with saturated sodium bicarbonate then washed with water (2×) and the aqueous layer was extracted with ethyl acetate (2×). The combined organic layers were washed with brine and dried over anhydrous Na₂SO₄, filtered concentrated under reduced pressure, The crude compound was purified by column chromatography (0-80% EtOAc in PE) to give tert-butyl (S)-2-(3-bromo-4-fluorophenyl)-4-methyl-3-(2-oxo-2,3-dihydro-1H-imidazol-1-yl)-2,4,6,7-tetrahydro-5H-pyrazolo[4,3-c]pyridine-5-carboxylate (3.0 g, 5.18 mmol) as a yellow solid. m/z (ESI-pos) M+1=492.0

Step F: To a mixture of tert-butyl (S)-2-(3-bromo-4-fluorophenyl)-4-methyl-3-(2-oxo-2,3-dihydro-1H-imidazol-1-yl)-2,4,6,7-tetrahydro-5H-pyrazolo[4,3-c]pyridine-5-carboxylate (2.0 g, 4.07 mmol), 2-allyl-4,4,5,5-tetramethyl-1,3,2-dioxaborolane (2.05 g, 12.21 mmol) and CsF (6.18 g, 40.7 mmol) in THF (150 mL) was added Pd(PPh₃)₄ (470 mg, 0.407 mmol) at 25° C. under N₂. The resulting mixture was stirred at 80° C. for 16 h. The reaction mixture was cooled to room temperature, and diluted with ethyl acetate and washed with water (3×). The combined aqueous layers were extracted with ethyl acetate (3×). The combined organic layers were washed with brine (3×), dried over anhydrous Na₂SO₄, filtered and concentrated. The crude compound was purified by column chromatography (0-80% EtOAc in PE) to give tert-butyl (S)-2-(3-allyl-4-fluorophenyl)-4-methyl-3-(2-oxo-2,3-dihydro-1H-imidazol-1-yl)-2,4,6,7-tetrahydro-5H-pyrazolo[4,3-c]pyridine-5-carboxylate (2.0 g, 3.53 mmol) as a yellow oil. m/z (ESI-pos) M+1=454.1.

Step G: To a solution of tert-butyl (S)-2-(3-allyl-4-fluorophenyl)-4-methyl-3-(2-oxo-2,3-dihydro-1H-imidazol-1-yl)-2,4,6,7-tetrahydro-5H-pyrazolo[4,3-c]pyridine-5-carboxylate (2.0 g, 4.41 mmol) and 1-allyl-2-bromobenzene (1.13 g, 5.73 mmol) in N-methyl-2-pyrrolidone (50 mL) was added (1R,2R)-1,2-bis(methylamino)cyclohexane (628 mg, 4.41 mmol), CuI (840 mg, 4.41 mmol) and K₂CO₃ (1.82 g, 13.23 mmol). The reaction mixture was heated at 130° C. for 3 h under the N₂ and then cooled to ambient temperature, diluted with ethyl acetate, and washed with water (3×). The combined aqueous layers were extracted with ethyl acetate (3×). The combined organic layers were washed with brine, dried over anhydrous Na₂SO₄, filtered and evaporated under reduced pressure. The crude product was purified by column chromatography (0-100% EtOAc in PE) to give tert-butyl (S)-2-(3-allyl-4-fluorophenyl)-3-(3-(2-allylphenyl)-2-oxo-2,3-dihydro-1H-imidazol-1-yl)-4-methyl-2,4,6,7-tetrahydro-5H-pyrazolo[4,3-c]pyridine-5-carboxylate (580 mg) as a yellow solid. m/z (ESI-pos) M+1=570.3

Step H: To a solution of tert-butyl (S)-2-(3-allyl-4-fluorophenyl)-3-(3-(2-allylphenyl)-2-oxo-2,3-dihydro-1H-imidazol-1-yl)-4-methyl-2,4,6,7-tetrahydro-5H-pyrazolo[4,3-c]pyridine-5-carboxylate (500 mg, 0.87 mmol) in DCE (500 mL) was added Hoveyda-Grubbs II (500 mg, 0.79 mmol) at 25° C. under N₂. The reaction mixture was heated at 40° C. for 16 h under N₂. After that, the reaction mixture was cooled to ambient temperature and quenched with water, and the aqueous layer was extracted with dichloromethane (3×). The combined organic layers were washed with brine solution, dried over anhydrous Na₂SO₄, filtered and concentrated. The crude compound was purified by column chromatography (0-100% EtOAc in PE) to give tert-butyl (S)-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (430 mg) as a yellow solid. m/z (ESI-pos) M+1=542.2

Step I: To a solution of tert-butyl (S)-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (430 mg, 0.79 mmol) in THF (8 mL) was added Pd/C (215 mg, 50% w/w) at 25° C. under a H₂ atmosphere. The mixture was stirred at 25° C. for 16 h. The reaction mixture was filtered through celite and the filtrate was evaporated under reduced pressure to give tert-butyl (S)-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (290 mg, 0.53 mmol) as a yellow solid. m/z (ESI-pos) M+1=544.3

Step J: tert-butyl (S)-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (290 mg, 0.53 mmol) was dissolved in HCl/1,4-dioxane (4M, 10 mL) and stirred at 25° C. for 1 h. The reaction mixture was directly concentrated under reduced pressure to give (S)-26-fluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (330 mg crude, 0.53 mmol) as a yellow solid. m/z (ESI-pos) M+1=444.2

Step K: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (217 mg, 0.53 mmol) in dimethylformamide (10 mL) were added DIEA (683 mg, 5.3 mmol) and HATU (302 mg, 0.795 mmol), the mixture stirred at ambient temperature for 10 min, then (S)-26-fluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-on hydrogen chloride (330 mg, 0.53 mmol) was added to the mixture. The mixture was stirred overnight. The reaction mixture was diluted with ethyl acetate and washed with water (2×). The aqueous layer was extracted with ethyl acetate (2×) and the combined organic layers were washed with brine, dried over anhydrous Na₂SO₄, filtered and the filtrate was evaporated under reduced pressure. The residue was purified by column chromatography (0-10% MeOH in DCM) to give the compound, which was further purified by prep-HPLC [Waters MS-triggered Prep-LC with QDA detector, WELCH Xtimate C18 21.2*250 mm 10 μm, eluted with 60-100% ACN in H₂O (0.1% FA)] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (135 mg) as a white solid. m/z (ESI-pos) M+1=837.3; ¹H NMR (400 MHz, DMSO) δ 10.28 (s, 1H), 7.54-7.48 (m, 2H), 7.43-7.38 (m, 1H), 7.34-7.19 (m, 6H), 7.08-7.02 (m, 1H), 6.86-6.68 (m, 3H), 5.64-5.55 (m, 1H), 4.48-4.28 (m, 1H), 3.73 (d, J=8.8 Hz, 2H), 3.55-3.42 (m, 1H), 3.04-2.89 (m, 1H), 2.83-2.65 (m, 4H), 1.78-1.43 (m, 13H), 1.27 (s, 3H), 1.24-1.151 (m, 9H).

### Example 35—Synthesis of Compound 35

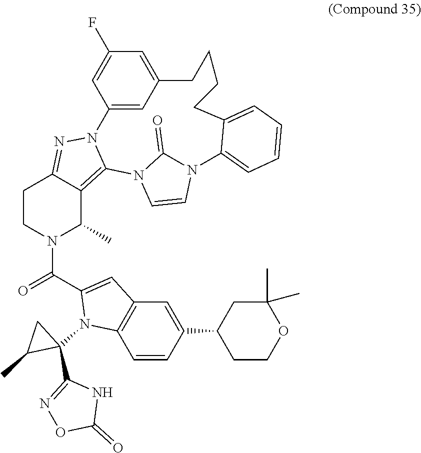

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-27-fluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 35 was synthesized following the procedure of Example 34, with 3-bromo-4-fluoroaniline being replaced with 3-bromo-5-fluoroaniline in step A. m/z (ESI-pos) M+1=837.3; ¹H NMR (400 MHz, DMSO) δ 11.53 (s, 1H), 7.57-7.15 (m, 8H), 7.13-6.61 (m, 5H), 5.60 (s, 1H), 4.41 (s, 1H), 3.76-3.68 (m, 2H), 3.64-3.32 (s, 1H), 3.03-2.52 (m, 7H), 1.85-1.49 (m, 10H), 1.48-1.33 (m, 2H), 1.33-1.10 (m, 11H).

### Example 36—Synthesis of Compound 36

### Synthetic Scheme

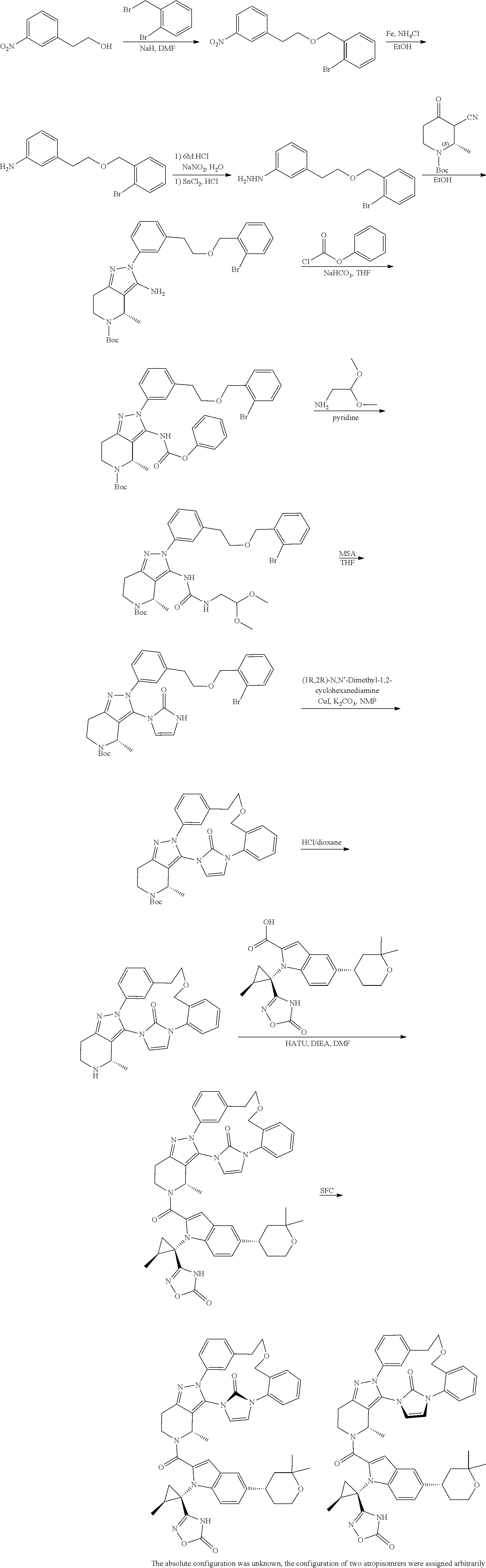

### Procedure for Synthesis of Compound 36

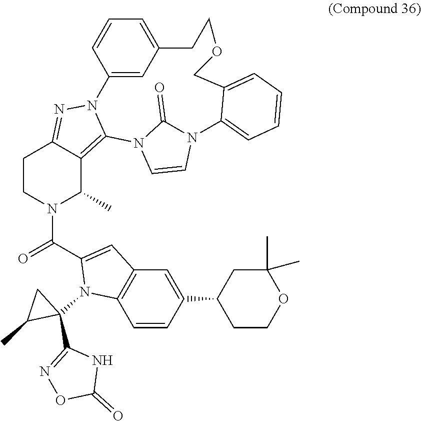

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a solution of 2-(3-nitrophenyl) ethan-1-ol (3.0 g, 17.94 mmol) in DMF (60 mL) was added NaH (60%, 5.38 g, 35.88 mmol) at 0° C. under N₂ and stirred for 45 min, then 1-bromo-2-(bromomethyl)benzene (5.38 g, 21.53 mmol) was added. The resulting mixture was stirred at 0° C. for 1 hr. The reaction mixture was quenched with saturated aqueous NH₄Cl, and diluted with ethyl acetate, washed with water (3×). The aqueous layer was extracted with ethyl acetate (3×). The combined organic layers were washed with brine (3×), dried over anhydrous Na₂SO₄, filtered and the filtrate was evaporated under reduced pressure. The crude compound was purified by column chromatography (0-50% EtOAc in PE) to give 1-bromo-2-((3-nitrophenethoxy)methyl)benzene (5.94 g, 15.9 mmol) as a yellow oil. m/z (ESI-pos) [M+23]=358.02.

Step B: To a solution of 1-bromo-2-((3-nitrophenethoxy)methyl)benzene (5.94 g, 17.73 mmol) in ethanol (100 mL) and water (20 mL) was added ammonium chloride (9.45 g, 177 mmol) and iron (4.93 g, 88.3 mmol) at 25° C. and the mixture was stirred at 70° C. for 4 h. The reaction mixture was cooled to ambient temperature and diluted with ethyl acetate, washed with water (3×) and the aqueous layer was extracted with ethyl acetate (3×). The combined organic layers were washed with brine, dried over anhydrous Na₂SO₄, filtered and concentrated. The crude compound was purified by column chromatography (0-80% EtOAc in PE) to give 3-(2-((2-bromobenzyl)oxy)ethyl)aniline (5.0 g, 14.7 mmol) as a yellow oil. m/z (ESI-pos) M+1=306.0/308.0.

Step C: A solution of sodium nitrite (1.24 g, 18.03 mmol) in 20 mL of water was added dropwise to a mixture of 3-(2-((2-bromobenzyl)oxy)ethyl)aniline (5.0 g, 16.39 mmol) and concentrated HCl (100 mL, 6 M) at 0° C. over 10 min. The resulting clear solution was stirred at 0° C. for 60 min, and then a solution of stannous chloride (6.21 g, 32.78 mmol) in concentrated HCl (10 mL) was added dropwise at 0° C. for 1.5 h. The mixture was stirred for 1 h at room temperature. The reaction mixture was diluted with water (200 mL) and adjusted to pH of about 10 with 5 N aq. NaOH. The resulting suspension was filtered and the filtrate was extracted with ethyl acetate (3×). The combined organic layers were washed with brine and dried over anhydrous Na₂SO₄, filtered and the filtrate was evaporated under reduced pressure. The crude compound was purified by column chromatography (0-10% MeOH in DCM) to give (3-(2-((2-bromobenzyl)oxy)ethyl)phenyl)hydrazine (1.7 g, 4.76 mmol) as a yellow solid. m/z (ESI-pos) M+1=321.1/323.1

Step D: To a mixture of (3-(2-((2-bromobenzyl)oxy)ethyl)phenyl)hydrazine (1.7 g, 5.31 mmol) in ethanol (40 mL) was added tert-butyl(2S)-3-cyano-2-methyl-4-oxopiperidine-1-carboxylate (1.15 g, 4.82 mmol) at 25° C. The resulting mixture was stirred at 80° C. for 8 h and then the reaction mixture was cooled down to room temperature, diluted with ethyl acetate and washed with water (3×). The aqueous layer was extracted with ethyl acetate (2×). The combined organic layers were washed with brine, dried over anhydrous Na₂SO₄, filtered and the filtrate was evaporated under reduced pressure. The crude compound was purified by column chromatography (0-100% EtOAc in PE) to give tert-butyl (S)-3-amino-2-(3-(2-((2-bromobenzyl)oxy)ethyl)phenyl)-4-methyl-2,4,6,7-tetrahydro-5H-pyrazolo[4,3-c]pyridine-5-carboxylate (1.69 g, 2.81 mmol) as a yellow solid. m/z (ESI-pos) M+1=541.2/543.2.

Step E: To a solution of tert-butyl (S)-3-amino-2-(3-(2-((2-bromobenzyl)oxy)ethyl)phenyl)-4-methyl-2,4,6,7-tetrahydro-5H-pyrazolo[4,3-c]pyridine-5-carboxylate (1.69 g, 3.12 mmol), sodium bicarbonate (786 mg, 9.36 mmol) in tetrahydrofuran (50 mL) was added benzenyl chloroformate (977 mg, 6.24 mmol). The reaction mixture was stirred at 25° C. for 2 h. The reaction mixture was directly concentrated under vacuum to give crude tert-butyl (S)-2-(3-(2-((2-bromobenzyl)oxy)ethyl)phenyl)-4-methyl-3-((phenoxycarbonyl)amino)-2,4,6,7-tetrahydro-5H-pyrazolo[4,3-c]pyridine-5-carboxylate (2.2 g) as a yellow oil, which was used directly for next step without further purification. m/z (ESI-pos) M+1=661.2/663.2.

Step F: Crude tert-butyl (S)-2-(3-(2-((2-bromobenzyl)oxy)ethyl)phenyl)-4-methyl-3-((phenoxycarbonyl)amino)-2,4,6,7-tetrahydro-5H-pyrazolo[4,3-c]pyridine-5-carboxylate (2.2 g, 3.12 mmol) was dissolved in pyridine (50 mL), to which 2,2-dimethoxyethylamine (984 mg, 9.36 mmol) was added. The mixture was stirred at 25° C. for 3 h and directly concentrated under vacuum to give the crude product which was purified by column chromatography (0-100% EtOAc in PE) to give tert-butyl (S)-2-(3-(2-((2-bromobenzyl)oxy)ethyl)phenyl)-3-(3-(2,2-dimethoxyethyl)ureido)-4-methyl-2,4,6,7-tetrahydro-5H-pyrazolo[4,3-c]pyridine-5-carboxylate (1.7 g, 2.53 mmol) as a yellow solid. m/z (ESI-pos) M+1=672.2/674.2.

Step G: To a solution of tert-butyl (S)-2-(3-(2-((2-bromobenzyl)oxy)ethyl)phenyl)-3-(3-(2,2-dimethoxyethyl)ureido)-4-methyl-2,4,6,7-tetrahydro-5H-pyrazolo[4,3-c]pyridine-5-carboxylate (1.7 g, 2.53 mmol) in tetrahydrofuran (100 mL) was added methanesulfonic acid (316 mg, 3.29 mmol). The mixture was stirred at 60° C. for 2 h and then cooled to ambient temperature, diluted with saturated sodium bicarbonate and ethyl acetate then washed with water (2×). The aqueous layer was extracted with ethyl acetate (2×) and the combined organic layers were washed with brine and dried over anhydrous Na₂SO₄, filtered and the filtrate was evaporated under reduced pressure. The crude compound was purified by column chromatography (0-80% EtOAc in PE) to give tert-butyl (S)-2-(3-(2-((2-bromobenzyl)oxy)ethyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (1.43 g, 2.11 mmol) as a yellow solid. m/z (ESI-pos) M+1=608.2/610.2.

Step H: To a solution of tert-butyl (S)-2-(3-(2-((2-bromobenzyl)oxy)ethyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (600 mg, 0.98 mmol) in N-methyl-2-pyrrolidone (30 mL) were added (1R,2R)-1,2-bis(methylamino)cyclohexane (210 mg, 1.48 mmol), CuI (188 mg, 0.98 mmol) and K₂CO₃ (408 mg, 2.96 mmol). The reaction mixture was heated at 130° C. for 3 h under the N₂ and then cooled to ambient temperature, diluted with ethyl acetate, and washed with water (3×). The aqueous layer was extracted with ethyl acetate (3×) and the combined organic layers were washed with brine, dried over anhydrous Na₂SO₄, filtered and evaporated under reduced pressure. The crude compound was purified by column chromatography (0-100% EtOAc in PE) to give tert-butyl (S)-8-methyl-30-oxo-22-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (338 mg) as a yellow solid. m/z (ESI-pos) M+1=528.3

Step I: tert-butyl (S)-8-methyl-30-oxo-22-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (150 mg, 0.28 mmol) was dissolved in HCl/1,4-dioxane (4M, 10 mL) and stirred at 25° C. for 1 h. The reaction mixture was directly concentrated under reduced pressure to give (S)-8-methyl-22-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (220 mg crude, 0.28 mmol) as a yellow solid. m/z (ESI-pos) M+1=428.3

Step J: To a solution of 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (146 mg, 0.35 mmol) in dimethylformamide (8 mL) were added DIEA (451 mg, 3.5 mmol) and HATU (285 mg, 0.75 mmol). The mixture stirred for 10 min, then(S)-8-methyl-22-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (220 mg, 0.28 mmol) was added to the mixture. The mixture was stirred at ambient temperature overnight. The reaction mixture was diluted with ethyl acetate and washed with water (2×). The aqueous layer was extracted with ethyl acetate (3×) and the combined organic layers were washed with brine, dried over anhydrous Na₂SO₄, filtered and the filtrate was evaporated under reduced pressure to give the crude compound, which was purified by prep-HPLC [Instrument: Waters MS-triggered Prep-LC with QDA detector; Column: WELCH Xtimate C18 21.2*250 mm 10 μm; Gradient: 70-90% ACN in H₂O (0.1% FA) in 9 min; Flow rate: 25 mL/min] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (70 mg) as a yellow solid. m/z (ESI-pos) M+1=821.3

Step K: (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (70 mg, 0.085 mmol) was dissolved in EtOH (120 mL) and separated by chiral supercritical fluid chromatography (separation condition: Daicel CHIRALPAK IA_4.6*250 mm, 5 μm; Mobile Phase: CO2/EtOH (0.1% DEA)=50/50 at 1.5 mL/min; Temp: 37° C.; Wavelength: 254 nm) to afford Isomer 1(24.7 mg) and Isomer 2 (44.4 mg) as white solids (The absolute configuration was unknown, the configuration of two atropisomers were assigned arbitrarily).

Isomer 1—m/z (ESI-pos) M+1=821.3; ¹H NMR (400 MHz, DMSO) δ 10.52 (s, 1H), 7.47-7.38 (m, 8H), 7.22-7.17 (m, 2H), 7.04 (s, 1H), 6.83-6.75 (m, 2H), 6.52 (d, J=3.2 Hz, 1H), 5.71-5.52 (m, 1H), 4.55-4.29 (m, 3H), 3.77-3.71 (m, 3H), 3.60-3.55 (m, 2H), 2.85-2.81 (m, 2H), 1.76-1.49 (m, 10H), 1.30-1.24 (m, 6H), 1.18-1.15 (m, 6H).

Isomer 2—m/z (ESI-pos) M+1=821.3; ¹H NMR (400 MHz, DMSO) δ 10.11 (s, 1H), 7.47-7.35 (m, 8H), 7.15-6.63 (m, 3H), 6.88-6.63 (m, 3H), 5.66-5.52 (m, 1H), 4.53-4.42 (m, 3H), 3.75-3.70 (m, 3H), 3.64-3.58 (m, 1H), 2.94-2.86 (m, 1H), 2.82-2.76 (m, 2H), 1.76-1.47 (m, 10H), 1.27 (s, 3H), 1.24-1.14 (m, 9H).

### Example 37—Synthesis of Compound 37

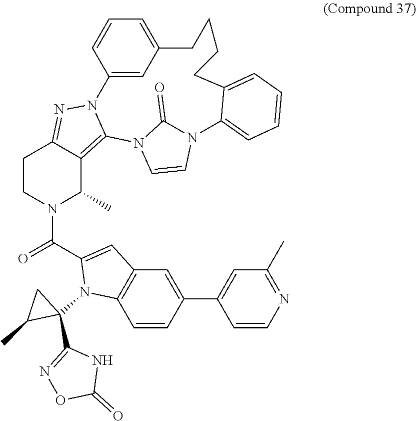

### (S)-8-methyl-7-({1-[(1S,2S)-2-methyl-1-(5-oxo-4,5-dihydro-1,2,4-oxadiazol-3-yl)cyclopropyl]-5-(2-methyl-4-pyridyl)-2-indolyl}carbonyl)-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 37 was synthesized following the procedure of Example 6, with 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid being replaced with 1-[(1S,2S)-2-methyl-1-(5-oxo-4,5-dihydro-1,2,4-oxadiazol-3-yl)cyclopropyl]-5-(2-methyl-4-pyridyl)-2-indolecarboxylic acid in step C. m/z (ESI-pos) M+1=798.3; ¹H NMR (400 MHz, DMSO) δ 11.76 (s, 1H), 8.46 (d, J=4.8 Hz, 1H), 8.07 (d, J=33.6 Hz, 1H), 7.68 (d, J=8.4 Hz, 1H), 7.58 (d, J=13.6 Hz, 2H), 7.51-7.38 (m, 3H), 7.37-7.12 (m, 5H), 7.06-6.62 (m, 4H), 5.63 (s, 1H), 4.33 (s, 1H), 3.52 (s, 1H), 2.94 (dd, J=16.0, 8.8 Hz, 1H), 2.86-2.59 (m, 5H), 2.53 (s, 3H), 1.73 (d, J=27.2 Hz, 4H), 1.47 (d, J=34.8 Hz, 3H), 1.33-1.10 (m, 6H).

### Example 38—Synthesis of Compound 38

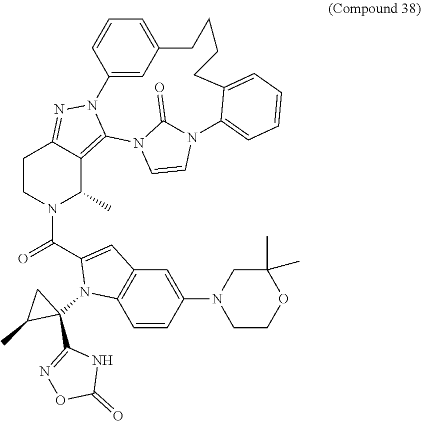

### (S)-7-{[5-(2,2-dimethyl-4-morpholinyl)-1-[(1S,2S)-2-methyl-1-(5-oxo-4,5-dihydro-1,2,4-oxadiazol-3-yl)cyclopropyl]-2-indolyl]carbonyl}-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 38 was synthesized following the procedure of Example 6, with 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid being replaced with 5-(2,2-dimethyl-4-morpholinyl)-1-[(1S,2S)-2-methyl-1-(5-oxo-4,5-dihydro-1,2,4-oxadiazol-3-yl)cyclopropyl]-2-indolecarboxylic acid in step C. m/z (ESI-pos) M+1=820.3; ¹H NMR (400 MHz, DMSO) δ 11.50 (s, 1H), 7.45-7.41 (m, 2H), 7.33-7.17 (m, 6H), 7.08-6.96 (m, 3H), 6.87-6.69 (m, 3H), 5.64-5.55 (m, 1H), 4.46-4.33 (m, 1H), 3.79-3.77 (m, 2H), 3.55-3.38 (m, 1H), 3.01-2.96 (m, 3H), 2.87-2.84 (m, 2H), 2.79-2.61 (m, 4H), 1.78-1.37 (m, 9H), 1.26 (s, 6H), 1.22-1.11 (m, 5H).

### Example 39—Synthesis of Compound 39

### Synthetic Scheme

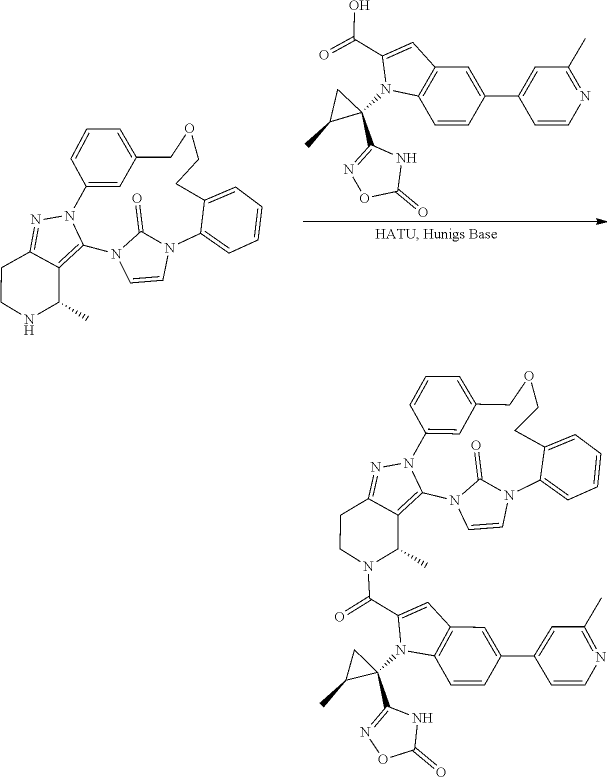

### Procedure for Synthesis of Compound 39

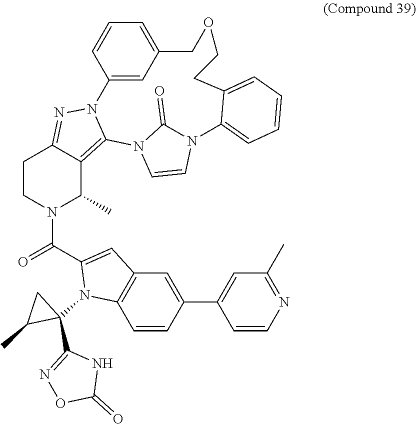

### (S)-8-methyl-7-({1-[(1S,2S)-2-methyl-1-(5-oxo-4,5-dihydro-1,2,4-oxadiazol-3-yl)cyclopropyl]-5-(2-methyl-4-pyridyl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a solution of(S)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (55 mg, 129 μmol) in dimethylformamide (2 mL) was added 1-[(1S,2S)-2-methyl-1-(5-oxo-4,5-dihydro-1,2,4-oxadiazol-3-yl)cyclopropyl]-5-(2-methyl-4-pyridyl)-2-indolecarboxylic acid (55.2 mg, 142 μmol) and N-ethyldiisopropylamine (50 mg, 386 μmol) and the mixture was stirred for 5 mins, then HATU (73.4 mg, 193 μmol) was added and the mixture was stirred at ambient temperature for 16 hours. The reaction mixture was diluted with ethyl acetate, washed with water (2×). The aqueous layer was extracted with ethyl acetate (2×). The combined organic layers were washed with brine solution, dried over anhydrous Na₂SO₄, filtered and the filtrate was evaporated under reduced pressure. The residue was purified by prep-HPLC [Instrument: Waters Prep-LC with UV detector; Column: Waters Luna C18 5 μm 21.2*250 mm; Gradient: 20-95% ACN in H₂O (0.1% FA) in 9.5 min; Flow rate: 20 mL/min] to give (S)-8-methyl-7-({1-[(1S,2S)-2-methyl-1-(5-oxo-4,5-dihydro-1,2,4-oxadiazol-3-yl)cyclopropyl]-5-(2-methyl-4-pyridyl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (17.6 mg) as a white solid. m/z (ESI-pos) M+1=798.3; ¹H NMR (400 MHz, DMSO) δ 11.76 (s, 1H), 8.46 (d, J=4.8 Hz, 1H), 8.07 (d, J=33.6 Hz, 1H), 7.68 (d, J=8.4 Hz, 1H), 7.58 (d, J=13.6 Hz, 2H), 7.51-7.38 (m, 3H), 7.37-7.12 (m, 5H), 7.06-6.62 (m, 4H), 5.63 (s, 1H), 4.33 (s, 1H), 3.52 (s, 1H), 2.94 (dd, J=16.0, 8.8 Hz, 1H), 2.86-2.59 (m, 5H), 2.53 (s, 3H), 1.73 (d, J=27.2 Hz, 4H), 1.47 (d, J=34.8 Hz, 3H), 1.33-1.10 (m, 6H).

### Example 40—Synthesis of Compound 40

### Synthetic Scheme

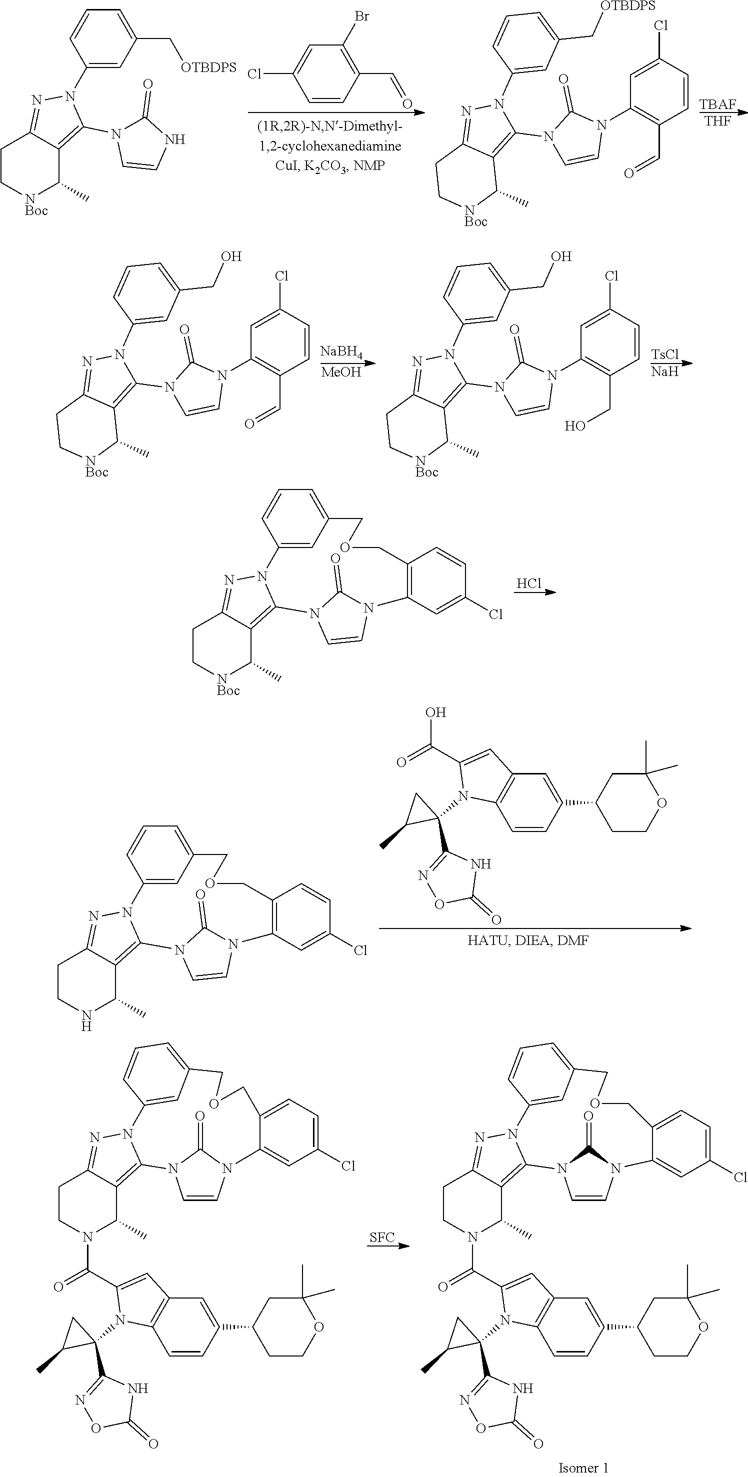

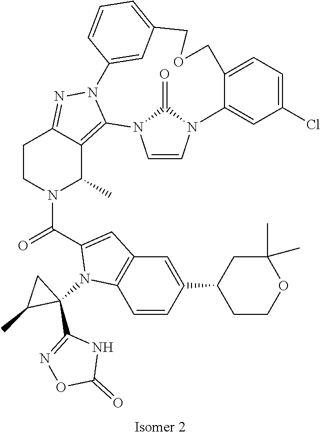

### Procedure for Synthesis of Compound 40

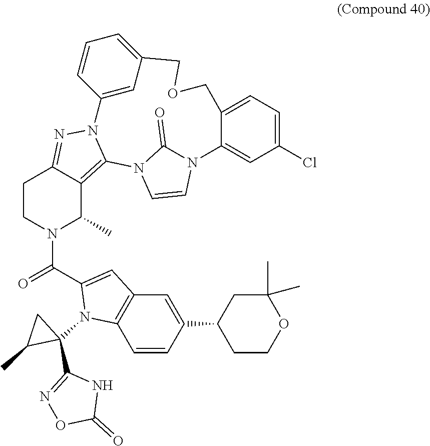

### (S)-17-chloro-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: A mixture of tert-butyl (S)-2-(m-{[tert-butylbis(phenyl)siloxy]methyl}phenyl)-4-methyl-3-(2-oxo-2,3-dihydro-1H-imidazol-1-yl)-1,2,5-triaza-2,4,6,7-tetrahydro-5H-indene-5-carboxylate (1.3 g, 1.96 mmol), 2-bromo-4-chlorobenzaldehyde (859 mg, 3.92 mmol), copper iodide (373 mg, 1.96 mmol), (1R,2R)-1,2-bis(methylamino)cyclohexane (557 mg, 3.92 mmol) and dipotassium carbonate (812 mg, 5.87 mmol) in N-methyl-2-pyrrolidone (26 mL, 269 mmol) was stirred at 100° C. for 16 h. The reaction mixture was then cooled to ambient temperature and diluted with ethyl acetate, washed with water (2×) and the aqueous layer was extracted with ethyl acetate (2×). The combined organic layers were washed with aqueous LiCl solution, dried over anhydrous Na₂SO₄, filtered and concentrated. The residue was purified by column chromatography (0-80% EtOAc in PE) to give tert-butyl (S)-2-(m-{[tert-butylbis(phenyl)siloxy]methyl}phenyl)-3-[3-(5-chloro-2-formylphenyl)-2-oxo-2,3-dihydro-1H-imidazol-1-yl]-4-methyl-1,2,5-triaza-2,4,6,7-tetrahydro-5H-indene-5-carboxylate (1 g, 1.25 mmol) as a yellow solid. m/z (ESI-pos) M+1=802.2.

Step B: To a solution of tert-butyl (S)-2-(m-{[tert-butylbis(phenyl)siloxy]methyl}phenyl)-3-[3-(5-chloro-2-formylphenyl)-2-oxo-2,3-dihydro-1H-imidazol-1-yl]-4-methyl-1,2,5-triaza-2,4,6,7-tetrahydro-5H-indene-5-carboxylate (1 g, 1.25 mmol) in tetrahydrofuran (20.8 mL) at 0° C. was added tetrabutylammonium fluoride (489 mg, 1.87 mmol) and the mixture was stirred for 1 hour. The reaction was quenched by H₂O and extracted with EtOAc (2×). The combined EtOAc layers were washed with brine, dried over Na₂SO₄, filtered and concentrated in vacuo. The crude product was purified by column chromatography (0-100% EtOAc in PE) to give tert-butyl (S)-3-[3-(5-chloro-2-formylphenyl)-2-oxo-2,3-dihydro-1H-imidazol-1-yl]-2-[m-(hydroxymethyl)phenyl]-4-methyl-1,2,5-triaza-2,4,6,7-tetrahydro-5H-indene-5-carboxylate (540 mg, 957 μmol) as a white solid. m/z (ESI-pos) M+1=564.2.

Step C: To a solution of tert-butyl (S)-3-[3-(5-chloro-2-formylphenyl)-2-oxo-2,3-dihydro-1H-imidazol-1-yl]-2-[m-(hydroxymethyl)phenyl]-4-methyl-1,2,5-triaza-2,4,6,7-tetrahydro-5H-indene-5-carboxylate (610 mg, 1.08 mmol) in methanol (10.2 mL) at 0° C. was added NaBH₄ (81.8 mg, 2.16 mmol). The reaction was stirred at 0° C. for 2 h, then diluted with ethyl acetate and washed with water (2×). The aqueous layer was extracted with ethyl acetate (2×) and the combined organic layers were washed with aqueous LiCl solution, dried over anhydrous Na₂SO₄, filtered and concentrated. The residue was purified by column chromatography (0-80% EtOAc in PE) to give tert-butyl (S)-3-{3-[5-chloro-2-(hydroxymethyl)phenyl]-2-oxo-2,3-dihydro-1H-imidazol-1-yl}-2-[m-(hydroxymethyl)phenyl]-4-methyl-1,2,5-triaza-2,4,6,7-tetrahydro-5H-indene-5-carboxylate (0.4 g, 707 μmol) as yellow solid. m/z (ESI-pos) M+1=566.2

Step D: To a solution of tert-butyl (S)-3-{3-[5-chloro-2-(hydroxymethyl)phenyl]-2-oxo-2,3-dihydro-1H-imidazol-1-yl}-2-[m-(hydroxymethyl)phenyl]-4-methyl-1,2,5-triaza-2,4,6,7-tetrahydro-5H-indene-5-carboxylate (0.4 g, 707 μmol) in tetrahydrofuran (40 mL) was added sodium hydride (106 mg, 1.77 mmol) at 0° C. The mixture was stirred at 0° C. for 20 min and then p-(chlorosulfonyl) toluene (67.4 mg, 353 μmol) in THF (2 mL) was added dropwise. The resulting mixture was stirred at 0° C. for 40 min and then allowed to warm to ambient temperature where it stirred overnight. The reaction mixture was quenched with water, extracted with ethyl acetate. The aqueous layer was extracted with ethyl acetate (2×) and the combined organic layers were washed with brine solution, dried over anhydrous Na₂SO₄, filtered and concentrated. The residue was purified by column chromatography (0-50% EtOAc in PE) to give tert-butyl (S)-17-chloro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (170 mg, 310 μmol) as a yellow solid. m/z (ESI-pos) M+1=548.2

Step E: tert-butyl (S)-17-chloro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (170 mg, 310 μmol) was dissolved in HCl/1,4-dioxane (4M, 1 mL) and stirred at 25° C. for 1 h. The mixture was directly concentrated to give (S)-17-chloro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (140 mg, 313 μmol) as a yellow solid, which was used directly in the next step. m/z (ESI-pos) M+1=448.2

Step F: To a solution of(S)-17-chloro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (130 mg, 290 μmol) in dimethylformamide (2 mL) was added N-ethyldiisopropylamine (150 mg, 1.16 mmol) and HATU (166 mg, 435 μmol). The mixture stirred for 10 min, then 5-[(S)-2,2-dimethyltetrahydro-4H-pyran-4-yl]-1-[(2S)-2-methyl-1-(5-oxo-4,5-dihydro-1,2,4-oxadiazol-3-yl)cyclopropyl]-2-indolecarboxylic acid (179 mg, 435 μmol) was added to the mixture and the mixture was stirred at ambient temperature overnight. The reaction mixture was diluted with ethyl acetate and washed with water (2×). The aqueous layer was extracted with ethyl acetate (2×). The combined organic layers were washed with brine, dried over anhydrous Na₂SO₄, filtered and concentrated. The crude residue was purified by prep-HPLC [Instrument: Waters MS-triggered Prep-LC with QDA detector; Column: Ultimate WELCH Xtimate C18 21.2*250 mm 10 μm; Gradient: 60-90% ACN in H₂O (0.1% FA) in 10 min; Flow rate: 25 mL/min] to give (S)-17-chloro-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (95 mg, 113 μmol) as a white solid, which was separated by SFC [Column: Daicel CHIRALPAK IA-H, 250 mm*30 mm I.D., 10 μm; Mobile phase: CO₂/MeOH [0.2% NH₃ (7M Solution in MeOH)]=50/50; Flow rate: 120 g/min] to get Isomer 1(25.8 mg) and Isomer 2 (58.7 mg) as white solids. (The absolute configuration was unknown, the configuration of two atropisomers were assigned arbitrarily)

Isomer 1: m/z (ESI-pos) M+1=841.2; ¹H NMR (400 MHz, DMSO) δ 11.50 (s, 1H), 7.73-7.45 (m, 6H), 7.40 (d, J=8.4 Hz, 1H), 7.25 (dd, J=13.2, 7.6 Hz, 2H), 7.12 (s, 1H), 6.88 (s, 1H), 6.75 (s, 1H), 6.57 (d, J=2.8 Hz, 1H), 5.64 (s, 1H), 4.69 (d, J=14.4 Hz, 1H), 4.56 (d, J=14.4 Hz, 1H), 4.43 (s, 1H), 4.14 (dd, J=25.6, 9.2 Hz, 2H), 3.72 (d, J=7.6 Hz, 2H), 3.59 (s, 1H), 2.89 (d, J=13.6 Hz, 1H), 1.81-1.44 (m, 10H), 1.38-1.04 (m, 11H).

Isomer 2: m/z (ESI-pos) M+1=841.2; ¹H NMR (400 MHz, DMSO) δ 11.65 (s, 1H), 7.65-7.36 (m, 8H), 7.25 (t, J=7.2 Hz, 2H), 7.10 (s, 1H), 6.81 (d, J=29.2 Hz, 2H), 5.67 (s, 1H), 4.59 (d, J=18.0 Hz, 2H), 4.43 (s, 1H), 4.19 (d, J=9.2 Hz, 1H), 4.06 (d, J=9.2 Hz, 1H), 3.72 (d, J=7.6 Hz, 2H), 3.60 (s, 1H), 2.91 (d, J=15.2 Hz, 1H), 1.78-1.48 (m, 10H), 1.31-1.13 (m, 11H).

### Example 41—Synthesis of Compound 41

### Synthetic Scheme

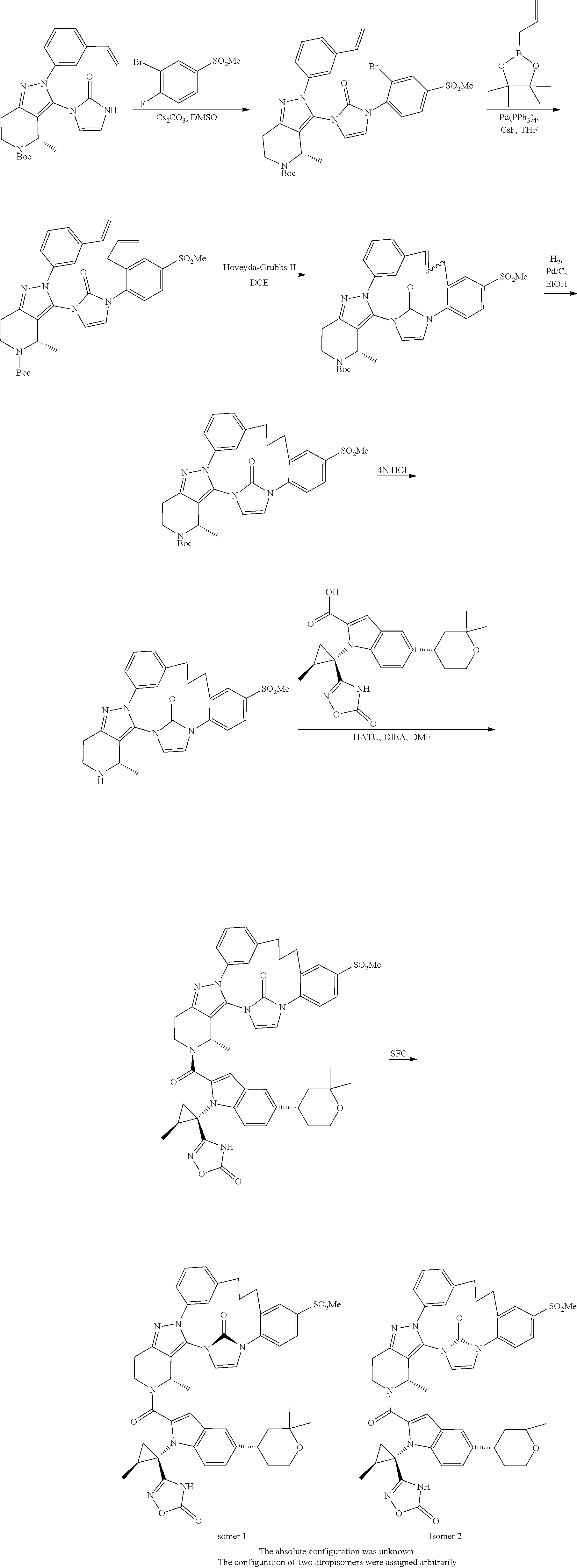

### Procedure for Synthesis of Compound 41

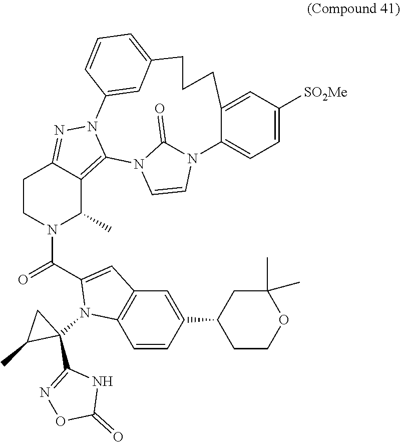

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18-mesyl-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a mixture of tert-butyl (S)-4-methyl-3-(2-oxo-2,3-dihydro-1H-imidazol-1-yl)-2-(3-vinylphenyl)-2,4,6,7-tetrahydro-5H-pyrazolo[4,3-c]pyridine-5-carboxylate (800 mg, 1.90 mmol) and Cesium carbonate (1.54 g, 4.75 mmol) in dimethyl sulfoxide (10 mL) was added 2-bromo-1-fluoro-4-(methylsulfonyl)benzene (622 mg, 2.47 mmol). The reaction mixture was heated at 60° C. where it stirred for 16 h under N₂. The reaction mixture was cooled to ambient temperature and diluted with ethyl acetate, then washed with water (3×) and the aqueous layer was extracted with ethyl acetate (3×). The combined organic layers were washed with brine, dried over anhydrous Na₂SO₄, filtered and evaporated under reduced pressure. The crude compound was purified by column chromatography (0-100% EtOAc in PE) to give tert-butyl (S)-3-(3-(2-bromo-4-(methylsulfonyl)phenyl)-2-oxo-2,3-dihydro-1H-imidazol-1-yl)-4-methyl-2-(3-vinylphenyl)-2,4,6,7-tetrahydro-5H-pyrazolo[4,3-c]pyridine-5-carboxylate (670 mg, 1.02 mmol) as a yellow solid. m/z (ESI-pos) M+1=654.1/656.1

Step B: To a mixture of tert-butyl (S)-3-(3-(2-bromo-4-(methylsulfonyl)phenyl)-2-oxo-2,3-dihydro-1H-imidazol-1-yl)-4-methyl-2-(3-vinylphenyl)-2,4,6,7-tetrahydro-5H-pyrazolo[4,3-c]pyridine-5-carboxylate (670 mg, 1.02 mmol), 2-allyl-4,4,5,5-tetramethyl-1,3,2-dioxaborolane (861 mg, 5.13 mmol) and CsF (619 mg, 4.08 mmol) in THF (20 mL) was added Pd(PPh₃)₄ (118 mg, 0.102 mmol) at 25° C. under N₂. The resulting mixture was stirred at 80° C. for 16 h under N₂. The reaction mixture was cooled down to room temperature, and diluted with ethyl acetate, then washed with water (3×). The combined aqueous layers were extracted with ethyl acetate (3×). The combined organic layers were washed with brine, dried over anhydrous Na₂SO₄, filtered and evaporated under reduced pressure. The crude compound was purified by column chromatography (0-60% EtOAc in PE) to give tert-butyl (S)-3-(3-(2-allyl-4-(methylsulfonyl)phenyl)-2-oxo-2,3-dihydro-1H-imidazol-1-yl)-4-methyl-2-(3-vinylphenyl)-2,4,6,7-tetrahydro-5H-pyrazolo[4,3-c]pyridine-5-carboxylate (438 mg, 0.64 mmol) as a yellow oil. m/z (ESI-pos) M+1=616.2

Step C: To a solution of tert-butyl (S)-3-(3-(2-allyl-4-(methylsulfonyl)phenyl)-2-oxo-2,3-dihydro-1H-imidazol-1-yl)-4-methyl-2-(3-vinylphenyl)-2,4,6,7-tetrahydro-5H-pyrazolo[4,3-c]pyridine-5-carboxylate (438 mg, 0.71 mmol) in DCE (438 mL) was added Hoveyda-Grubbs II (438 mg, 0.70 mmol) at 25° C. under N₂. The reaction mixture was heated at 80° C. for 3 h under N₂. The reaction mixture was cooled to ambient temperature and diluted with water. The mixture was extracted with dichloromethane (3×). The combined organic layers were washed with brine (2×), dried over anhydrous Na₂SO₄, filtered and evaporated under reduced pressure. The crude compound was purified by column chromatography (0-100% EtOAc in PE) to give tert-butyl (S)-18-mesyl-8-methyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,22,24,26-decaene-7-carboxylate (220 mg) as a yellow solid. m/z (ESI-pos) M+1=588.2

Step D: To a solution of tert-butyl (S)-18-mesyl-8-methyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,22,24,26-decaene-7-carboxylate (200 mg, 0.34 mmol) in ethanol (10 mL) was added Pd/C (100 mg, 50% w/w) at 25° C. The mixture was stirred at 25° C. for 16 h under a H₂ atmosphere. The reaction mixture was then filtered through a pad of Celite and the filtrate was evaporated under reduced pressure to give tert-butyl (S)-18-mesyl-8-methyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (180 mg) as a yellow solid. m/z (ESI-pos) M+1=590.3.

Step E: tert-butyl (S)-18-mesyl-8-methyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (180 mg, 0.305 mmol) was dissolved in HCl/1,4-dioxane (4M, 4 mL) and stirred at 25° C. for 1 h. The reaction mixture was directly concentrated under reduced pressure to give (S)-18-mesyl-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (150 mg, 0.26 mmol) as a yellow solid. m/z (ESI-pos) M+1=490.1

Step F: To a solution of 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (126 mg, 0.306 mmol) in dimethylformamide (5 mL) were added DIEA (396 mg, 3.06 mmol) and HATU (175 mg, 0.46 mmol). The mixture stirred for 10 min, then(S)-18-mesyl-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (150 mg, 0.26 mmol) was added to the mixture. The mixture was stirred at ambient temperature overnight. The reaction mixture was diluted with ethyl acetate and washed with water (3×). The aqueous layer was extracted with ethyl acetate (2×). The combined organic layers were washed with brine, dried over anhydrous Na₂SO₄, filtered and the filtrate was evaporated under reduced pressure to give the crude compound, which was purified by prep-HPLC [Instrument: GILSON Prep LC with UV detector; Column: XBridge Prep C18 19*150 mm 5 μm; Gradient: eluted with 62-95% ACN in H₂O (0.1% NH₄HCO₃) in 10 min; Flow rate: 15 ml/min] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18-mesyl-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (85 mg) as a yellow solid. m/z (ESI-pos) M+1=883.4

Step G: (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18-mesyl-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (85 mg, 0.11 mmol) was dissolved in EtOH (150 mL) and separated by chiral supercritical fluid chromatography [Apparatus: Gilson281; Column: Daicel CHIRALPAK IB, 250 mm*30 mm I.D., 10 μm; Mobile phase: Hex/EtOH [0.2% NH₃ (7M Solution in MeOH)]=40/60; Flow rate: 25 ml/min]; Temp: 35° C.; Wavelength: 254 nm] to afford Isomer 1 (27.4 mg) and Isomer 2 (40.6 mg) as white solids (The absolute configuration was unknown, the configuration of two atropisomers were assigned arbitrarily.

Isomer 1: m/z (ESI-pos) M+1=883.4; ¹H NMR (400 MHz, DMSO) δ 11.57 (s, 1H), 7.98 (s, 1H), 7.92-7.87 (m, 1H), 7.66 (d, J=8.0 Hz, 1H), 7.51 (s, 1H), 7.43-7.37 (m, 3H), 7.24-7.19 (m, 2H), 7.09 (s, 1H), 6.83-6.66 (m, 3H), 5.71-5.48 (m, 1H), 4.52-4.34 (m, 1H), 3.76-3.71 (m, 2H), 3.63-3.45 (m, 1H), 3.23 (s, 3H), 2.99-2.96 (m, 2H), 2.91-2.73 (m, 4H), 2.28-2.17 (m, 1H), 2.00-1.48 (m, 15H), 1.19 (s, 6H).

Isomer 2: m/z (ESI-pos) M+1=883.4; ¹H NMR (400 MHz, DMSO) δ 11.14 (s, 1H), 8.02-7.99 (m, 1H), 7.90-7.86 (m, 1H), 7.73-7.68 (m, 1H), 7.48 (s, 1H), 7.43-7.34 (m, 3H), 7.24-7.20 (m, 2H), 7.10 (s, 1H), 6.86-6.80 (m, 1H), 6.71-6.69-0 (m, 1H), 6.65-6.62 (m, 1H), 5.68-5.57 (m, 1H), 4.46-4.29 (m, 1H), 3.73-3.70 (m, 2H), 3.60-3.46 (m, 1H), 3.24 (s, 3H), 3.02-2.98 (m, 2H), 2.92-2.72 (m, 4H), 2.26-2.17 (m, 1H), 2.02-1.52 (m, 15H), 1.18 (s, 6H).

### Example 42—Synthesis of Compound 42

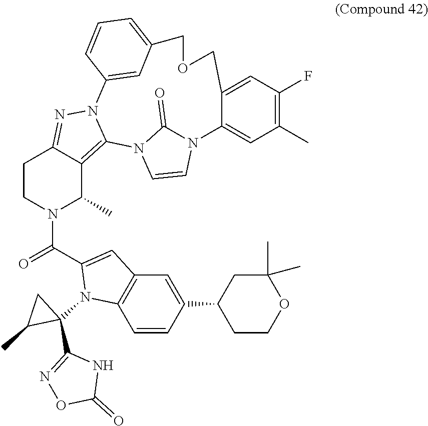

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18-fluoro-8,17-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 42 was synthesized following the procedure of Example 40, with 2-bromo-4-chlorobenzaldehyde being replaced with 5-bromo-2-fluoro-4-tolualdehyde in step A.

Isomer 1: m/z (ESI-pos) M+1=839.3; ¹H NMR (400 MHz, DMSO) δ 11.43 (s, 1H), 7.56 (d, J=8.0 Hz, 1H), 7.49 (dd, J=12.8, 5.2 Hz, 2H), 7.43-7.30 (m, 3H), 7.24 (t, J=7.2 Hz, 2H), 7.11 (s, 1H), 6.76 (d, J=59.6 Hz, 3H), 5.67 (s, 1H), 4.64-4.55 (m, 2H), 4.53-4.25 (m, 1H), 4.14 (d, J=9.6 Hz, 1H), 4.00 (d, J=9.2 Hz, 1H), 3.75-3.69 (m, 2H), 3.57 (s, 1H), 2.90 (d, J=16.0 Hz, 1H), 2.32-2.24 (m, 3H), 1.77-1.43 (m, 10H), 1.32-1.10 (m, 11H).

Isomer 2: m/z (ESI-pos) M+1=839.3; ¹H NMR (400 MHz, DMSO) δ 10.78 (s, 1H), 7.58-7.43 (m, 4H), 7.37 (dd, J=19.6, 8.4 Hz, 2H), 7.30-7.11 (m, 3H), 6.86 (s, 1H), 6.69 (d, J=2.4 Hz, 1H), 6.55 (d, J=3.2 Hz, 1H), 5.64 (s, 1H), 4.69 (d, J=14.8 Hz, 1H), 4.54 (d, J=14.4 Hz, 1H), 4.40 (s, 1H), 4.09 (dd, J=25.6, 9.2 Hz, 2H), 3.73 (dd, J=19.2, 12.0 Hz, 2H), 3.57 (s, 1H), 2.89 (dd, J=19.2, 26.0 Hz, 1H), 2.35-2.17 (m, 3H), 1.79-1.45 (m, 10H), 1.31-1.15 (m, 11H).

### Example 43—Synthesis of Compound 43

### Synthetic Scheme

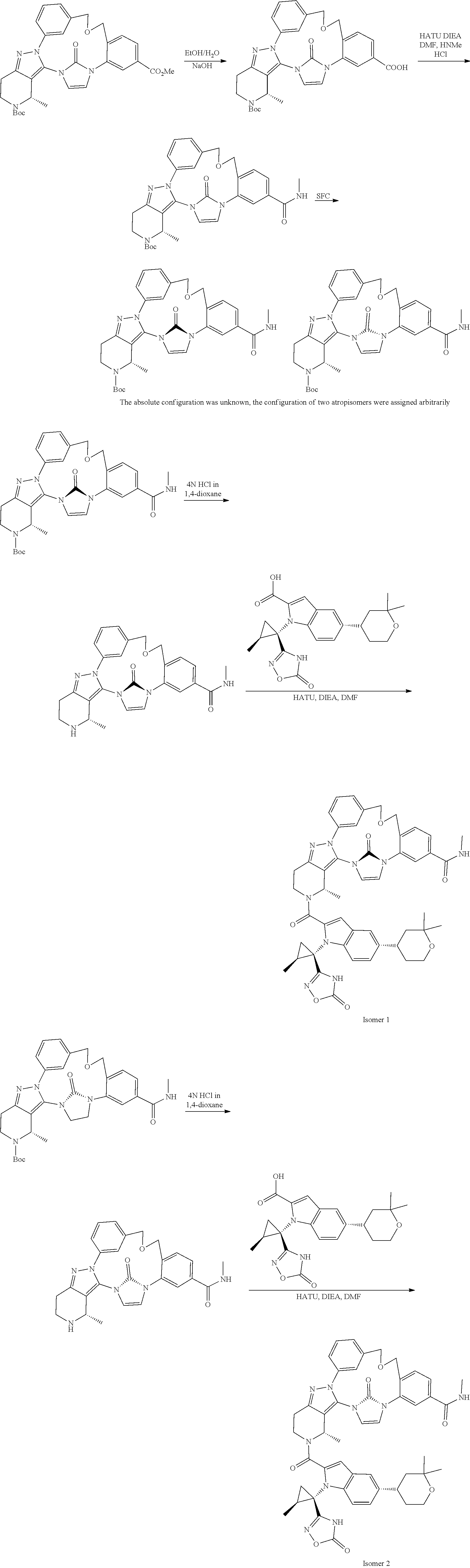

### Procedure for Synthesis of Compound 43

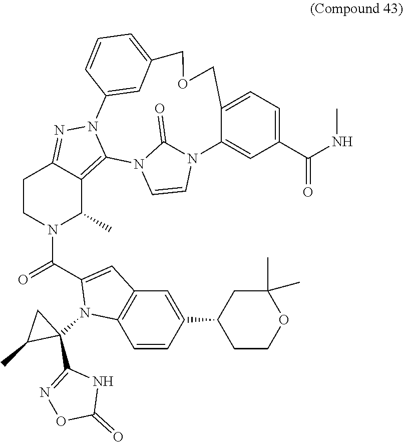

### N-methyl-(S)-7-({5-[(R)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-17-carboxamide

Step A: tert-butyl (S)-17-methoxycarbonyl-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate was prepared following the procedure of Example 40, with 2-bromo-4-chlorobenzaldehyde being replaced with methyl 3-bromo-4-formylbenzoate in step A. m/z (ESI-pos) M+1=572.2.

Step B: To a solution of tert-butyl (S)-17-methoxycarbonyl-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (560 mg, 980 μmol) in EtOH (10 mL) and water (2 mL) was added sodium hydroxide (196 mg, 4.9 mmol). The mixture was stirred at ambient temperature for 1 h. The reaction was quenched with saturated aqueous citric acid and then extracted with EtOAc (3×). The combined organic layers were washed with brine, dried over Na₂SO₄, filtered and concentrated to give (S)-7-tert-butoxycarbonyl-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-17-carboxylic acid (500 mg, 673 μmol) as a yellow solid. m/z (ESI-pos) M+1=558.2.

Step C: To a solution of(S)-7-tert-butoxycarbonyl-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-17-carboxylic acid (320 mg, 574 μmol), methylamine hydrochloride (356 mg, 11.5 mmol) in DMF (8 mL) at ambient temperature were added DIEA (2.23 g, 17.2 mmol) and HATU (327 mg, 861 μmol). The mixture was stirred for 16 h and then diluted with water and extracted with EtOAc (2×). The combined EtOAc layers were washed with brine, dried over Na₂SO₄, filtered and concentrated in vacuo. The crude product was purified by column chromatography (0-5% MeOH in DCM) to give tert-butyl (S)-8-methyl-17-(N-methylcarbamoyl)-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (80 mg, 126 μmol) as a yellow solid. m/z (ESI-pos) M+1=571.2.

Step D: A solution of tert-butyl (S)-8-methyl-17-(N-methylcarbamoyl)-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (80 mg, 126 μmol) in MeOH (100 mL) was separated by chiral supercritical fluid chromatography [separation condition: Daicel CHIRALPAK IH_3, 3.0*150 mm, 3 um; Mobile Phase: CO2/MeOH (0.1% DEA)=80/20 at 80 g/min; Temp: 37° C.; Wavelength: 214 nm] to afford Isomer A (18 mg) and Isomer B (45 mg) as yellow solids. (The absolute configuration was unknown, the configuration of two atropisomers were assigned arbitrarily).

Step E: To a solution of tert-butyl (S)-8-methyl-17-(N-methylcarbamoyl)-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (Isomer B, 45 mg, 70.1 μmol) in DCM (5 mL) was added HCl/1,4-dioxane (4M, 5 mL). The reaction mixture was stirred at ambient temperature for 1 hour and then directly concentrated in vacuo to get N-methyl-(S)-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-17-carboxamide hydrochloride (45 mg, 88.79 μmol) as a yellow solid. m/z (ESI-pos) M+1=471.3.

Step F: To a solution of N-methyl-(S)-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-17-carboxamide hydrochloride (45 mg, 88.79 μmol) in DMF (5 mL) were added 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (32 mg, 78.6 μmol) and DIEA (102 mg, 786 μmol). The reaction mixture was stirred at ambient temperature for 10 mins and then HATU (44.8 mg, 118 μmol) was added. The mixture was stirred for 16 hours and then diluted with water and extracted with EtOAc (3×). The combined EtOAc layers were washed with brine, dried over Na₂SO₄, filtered and concentrated in vacuo. The crude product was purified by column chromatography (0-5% MeOH in DCM) to give 50 mg of the desired product, which was further purified by prep-HPLC [Instrument: Waters Prep-LC with UV detector; Column: Waters Luna C18 10 μm 21.2*250 mm; Gradient: eluted with 50-85% ACN in H₂O (0.1% FA) in 10.5 min; Flow rate: 20 mL/min] to give N-methyl-(S)-7-({5-[(R)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-17-carboxamide (Isomer 2, 5.39 mg, 6.24 μmol) as a white solid. m/z (ESI-pos) M+1=864.3; ¹HNMR (400 MHz, DMSO) δ 11.59 (s, 1H), 8.35 (s, 1H), 7.96-7.88 (m, 2H), 7.66 (d, J=7.6 Hz, 1H), 7.57-7.48 (m, 3H), 7.40 (d, J=8.4 Hz, 1H), 7.28-7.21 (m, 2H), 7.14 (s, 1H), 6.87 (s, 1H), 6.78-6.74 (m, 1H), 6.60 (d, J=3.2 Hz, 1H), 5.71-5.52 (m, 1H), 4.71-4.56 (m, 2H), 4.47-4.37 (m, 1H), 4.22-4.14 (m, 2H), 3.75-3.70 (m, 2H), 3.61-3.50 (m, 1H), 2.99-2.86 (m, 2H), 2.80 (d, J=4.4 Hz, 3H), 1.71-1.47 (m, 10H), 1.30-1.24 (m, 4H), 1.18 (s, 6H).

Step G: Isomer A (18 mg) was also carried through steps E and F to give N-methyl-(S)-7-({5-[(R)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-17-carboxamide (Isomer 1, 14.6 mg, 17.00 μmol) as a white solid. m/z (ESI-pos) M+1=864.3; ¹HNMR (400 MHz, DMSO) δ 11.64 (s, 1H), 8.32 (s, 1H), 7.92-7.85 (m, 2H), 7.63-7.57 (m, 2H), 7.52-7.47 (m, 2H), 7.41 (d, J=8.4 Hz, 1H), 7.27-7.23 (m, 2H), 7.10 (s, 1H), 6.85-6.63 (m, 3H), 5.72-5.59 (m, 1H), 4.61-4.38 (m, 3H), 4.25 (d, J=9.6 Hz, 1H), 4.10 (d, J=9.2 Hz, 1H), 3.73-3.71 (m, 2H), 3.61-3.50 (m, 1H), 3.00-2.89 (m, 2H), 2.81 (d, J=4.4 Hz, 3H), 1.74-1.50 (m, 10H), 1.30-1.24 (m, 4H), 1.19 (s, 6H).

### Example 44—Synthesis of Compound 44

### Synthetic Scheme

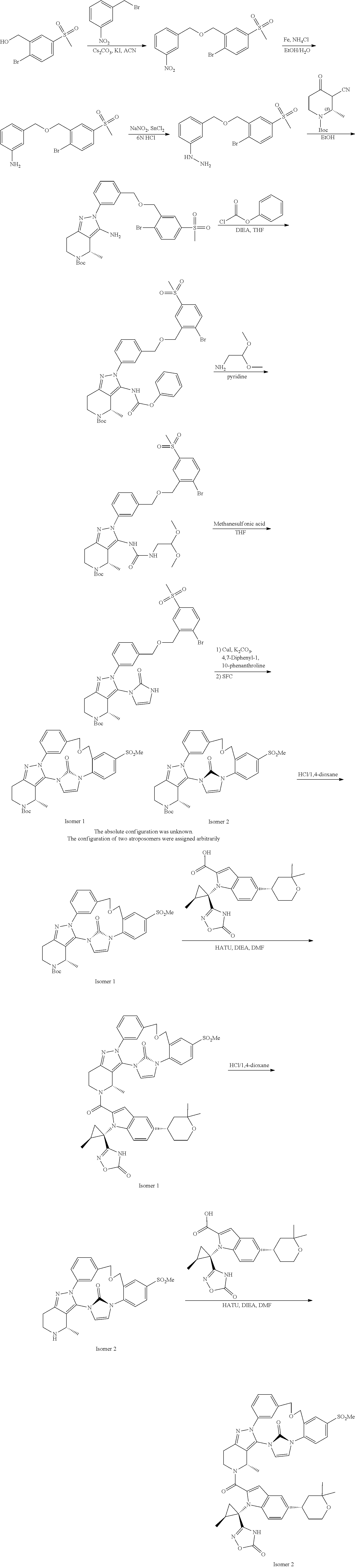

### Procedure for Synthesis of Compound 44

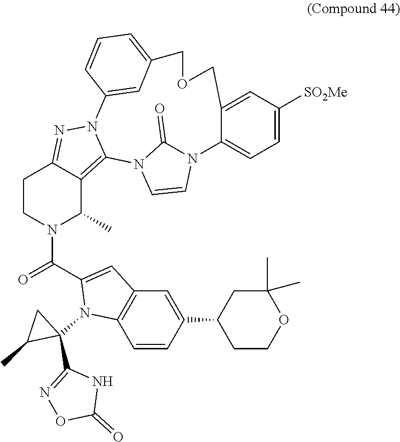

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18-mesyl-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (2-bromo-5-(methylsulfonyl)phenyl) methanol (2.97 g, 11.2 mmol) in acetonitrile (150 mL) were added 1-(bromomethyl)-3-nitrobenzene (2.42 g, 11.2 mmol), potassium iodide (0.372 g, 2.24 mmol) and Cs₂CO₃ (10.9 g, 33.6 mmol). The mixture was stirred at 40° C. for 16 hours and then concentrated under vacuum to remove the solvent, the residue was diluted with water and extracted with EtOAc (2×). The combined EtOAc layers were washed with brine (2×), dried over Na₂SO₄, filtered and concentrated in vacuo. The crude residue was purified by column chromatography (0-30% EtOAc in PE) to give 1-bromo-4-(methylsulfonyl)-2-(((3-nitrobenzyl)oxy)methyl)benzene (3.49 g) as a yellow solid. m/z (ESI-pos) M+1=399.9, 402.0.

Step B: To a solution of 1-bromo-4-(methylsulfonyl)-2-(((3-nitrobenzyl)oxy)methyl)benzene (3.49 g, 8.72 mmol) in ethanol (120 mL) were added iron (2.43 g, 43.6 mmol), ammonium chloride (4.67 g, 87.2 mmol) and water (40 mL). The mixture was stirred at 80° C. for 16 hours and then concentrated to remove EtOH. The residue was diluted with water and extracted with EtOAc (2×). The combined EtOAc layers were washed with brine (2×), dried over Na₂SO₄, filtered and concentrated in vacuo. The crude product was purified by column chromatography (0-20% EtOAc in PE) to give 3-(((2-bromo-5-(methylsulfonyl)benzyl)oxy)methyl)aniline (2.78 g, 4.5 mmol) as a colorless oil. m/z (ESI-pos) M+1=370.0, 372.0.

Step C: To a solution of 3-(((2-bromo-5-(methylsulfonyl)benzyl)oxy)methyl)aniline (2.78 g, 7.53 mmol) in aq. HCl (6N., 90 mL) at 0° C. was added sodium nitrite (572 mg, 8.28 mmol) in H₂O (5 mL). The mixture was stirred at 0° C. for 1 hour and then SnCl₂ (2.85 g, 15.06 mmol) in aq. HCl (6N, 10 mL) was added. The resulting mixture was warm to ambient temperature and stirred for 1 hour and then quenched the reaction with aq. NaOH (10 N, 40 mL) to adjust pH about 8-9. The mixture was then extracted with EtOAc (3×). The combined organics were washed with brine, dried over Na₂SO₄, filtered and the filtrate was concentrated in vacuo. The crude product was purified by column chromatography (0-100% EtOAc in PE) to give (3-(((2-bromo-5-(methylsulfonyl)benzyl)oxy)methyl)phenyl)hydrazine (2.37 g, 6.17 mmol) as a yellow solid. m/z (ESI-pos) M+1=385.0, 387.0.

Step D: To a solution of (3-(((2-bromo-5-(methylsulfonyl)benzyl)oxy)methyl)phenyl) hydrazine (2.37 g, 6.17 mmol) in ethanol (30 mL) was added tert-butyl(2S)-3-cyano-2-methyl-4-oxo-1-piperidinecarboxylate (1.47 g, 6.17 mmol). The mixture was stirred at 80° C. for 16 hours. The reaction mixture was directly concentrated to get the crude product, which was purified by column chromatography (0-80% EtOAc in PE) to give tert-butyl (S)-3-amino-2-(3-(((2-bromo-5-(methylsulfonyl)benzyl)oxy)methyl)phenyl)-4-methyl-2,4,6,7-tetrahydro-5H-pyrazolo[4,3-c]pyridine-5-carboxylate (1.5 g, 2.23 mmol) as a yellow solid. m/z (ESI-pos) M+1=605.2, 607.1.

Step E: To a solution of tert-butyl (S)-3-amino-2-(3-(((2-bromo-5-(methylsulfonyl)benzyl)oxy)methyl)phenyl)-4-methyl-2,4,6,7-tetrahydro-5H-pyrazolo[4,3-c]pyridine-5-carboxylate (1.5 g, 2.48 mmol) in THF (60 mL) were added DIEA (960 mg, 7.44 mmol) and benzenyl chloroformate (776 mg, 4.96 mmol). The mixture was stirred at ambient temperature for 2 hours and then directly concentrated under reduced pressure to get crude tert-butyl (S)-2-(3-(((2-bromo-5-(methylsulfonyl)benzyl)oxy)methyl)phenyl)-4-methyl-3-((phenoxycarbonyl)amino)-2,4,6,7-tetrahydro-5H-pyrazolo[4,3-c]pyridine-5-carboxylate (2.3 g crude, 2.48 mmol) as a yellow solid, which was used directly to next step. m/z (ESI-pos) M+1=725.1, 727.1.

Step F: To a solution of crude tert-butyl (S)-2-(3-(((2-bromo-5-(methylsulfonyl)benzyl)oxy)methyl)phenyl)-4-methyl-3-((phenoxycarbonyl)amino)-2,4,6,7-tetrahydro-5H-pyrazolo[4,3-c]pyridine-5-carboxylate (2.3 g crude, 2.48 mmol) in pyridine (20 mL) at ambient temperature was added 2,2-dimethoxyethylamine (781 mg, 7.44 mmol). The mixture was stirred for 2 hours and then concentrated to get crude product, which was purified by column chromatography (0-100% EtOAc in PE) to give tert-butyl (S)-2-(3-(((2-bromo-5-(methylsulfonyl)benzyl)oxy)methyl)phenyl)-3-(3-(2,2-dimethoxyethyl)ureido)-4-methyl-2,4,6,7-tetrahydro-5H-pyrazolo[4,3-c]pyridine-5-carboxylate (1.2 g, 1.47 mmol) as a yellow solid. m/z (ESI-pos) M+1=736.2, 738.1.

Step G: To a solution of tert-butyl (S)-2-(3-(((2-bromo-5-(methylsulfonyl)benzyl)oxy)methyl)phenyl)-3-(3-(2,2-dimethoxyethyl)ureido)-4-methyl-2,4,6,7-tetrahydro-5H-pyrazolo[4,3-c]pyridine-5-carboxylate (1.2 g, 1.63 mmol) in THF (120 mL) was added methanesulfonic acid (235 mg, 2.45 mmol). The mixture was stirred at 60° C. for 2 hours and then cooled to ambient temperature, quenched with aq. NaHCO₃ (50 mL), extracted with EtOAc (2×). The combined organic layers were dried over anhydrous Na₂SO₄, filtered and concentrated under reduced pressure. The crude product was purified by column chromatography (0-100% EtOAc in PE) to give tert-butyl (S)-2-(3-(((2-bromo-5-(methylsulfonyl)benzyl)oxy)methyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (0.9 g, 1.2 mmol) as a yellow solid. m/z (ESI-pos) M+1=672.1, 674.1.

Step H: To a solution of tert-butyl (S)-2-(m-{[(2-bromo-4,5-difluorophenyl)methoxy]methyl}phenyl)-4-methyl-3-(2-oxo-2,3-dihydro-1H-imidazol-1-yl)-1,2,5-triaza-2,4,6,7-tetrahydro-5H-indene-5-carboxylate (680 mg, 971 μmol) in DMSO (56.7 mL) were added CuI (139 mg, 728 μmol), 4,7-Diphenyl-1,10-phenanthroline (323 mg, 971 μmol) and K₂CO₃ (335 mg, 2.43 mmol). The mixture was stirred at 120° C. for 24 hours. The mixture cooled to ambient temperature, diluted with water and extracted with EtOAc (2×). The combined organics were washed with brine (2×), dried over Na₂SO₄ and concentrated in vacuo. The crude product was purified by column chromatography (0-50% EtOAc in PE) to give racemic product (168 mg), which was further separated by chiral supercritical fluid chromatography [Column: Daicel CHIRALCEL OD, 3*150 mm, 3 um; Mobile Phase: CO₂/MeOH [0.1% DEA (7M Solution in MeOH)]=60/40; Flow rate: 1.5 mL/min; Temp: 35° C.; Wavelength: 214 nm] to afford(S)-18-mesyl-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 1, 134 mg) and(S)-18-mesyl-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 2, 56 mg). m/z (ESI-pos) M+1=592.2. (The absolute configuration was unknown, assigned arbitrarily).

Step I: To a solution of(S)-18-mesyl-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 1, 134 mg) in DCM (5 mL) was added HCl/1,4-dioxane (4M, 5 mL) at 0° C. The mixture was warmed to ambient temperature and stirred for 1 hour. The mixture was concentrated to give crude(S)-18-mesyl-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (Isomer 1, 110 mg, 224 μmol) as a yellow solid. m/z (ESI-pos) M+1=492.2.

Step J: To a solution of(S)-18-mesyl-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (Isomer 1, 110 mg, 224 μmol) in DMF (5 mL) were added 5-[(S)-2,2-dimethyltetrahydro-4H-pyran-4-yl]-1-[(2S)-2-methyl-1-(5-oxo-4,5-dihydro-1,2,4-oxadiazol-3-yl)cyclopropyl]-2-indolecarboxylic acid (92.1 mg, 224 μmol) and DIEA (289 mg, 2.24 mmol). The reaction mixture was stirred at ambient temperature for 10 min, followed by the addition of HATU (128 mg, 336 μmol). The mixture was stirred for 16 hours and then quenched with water, and extracted with EtOAc (2×). The combined EtOAc layers were washed with brine, dried over Na₂SO₄, filtered and concentrated in vacuo. The residue was purified by column chromatography (0-5% MeOH in DCM) to give the product, which was further purified by prep-HPLC [Instrument: Waters Prep-LC with UV detector; Column: Waters Luna C18 10 μm 21.2*250 mm; Gradient: eluted with 60-95% ACN in H2O (0.1% FA) in 12.7 min; Flow rate: 20 mL/min] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18-mesyl-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 1, 95.9 mg, 108 μmol) as a white solid. m/z (ESI-pos) M+1=885.2; ¹H NMR (400 MHz, DMSO) δ 8.14-8.05 (m, 1H), 8.07 (d, J=8.0 Hz, 1H), 7.76 (d, J=8.0 Hz, 1H), 7.61 (d, J=8.4 Hz, 1H), 7.52-7.41 (m, 2H), 7.43 (d, J=8.4 Hz, 1H), 7.31-7.23 (m, 2H), 7.11 (s, 1H), 6.83-6.63 (m, 2H), 5.82-5.58 (m, 1H), 4.68-4.61 (m, 2H), 4.51-4.33 (m, 2H), 4.22 (d, J=9.2 Hz, 1H), 3.75-3.70 (m, 2H), 3.61-3.47 (m, 1H), 3.27 (s, 3H), 2.96-2.89 (m, 2H), 1.73-1.55 (m, 10H), 1.34-1.26 (m, 4H), 1.21 (s, 6H).

Step K: Steps I and J were repeated, with (Isomer 2, 56 mg) to afford(S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18-mesyl-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 2, 14.7 mg, 16.6 μmol) as a white solid. m/z (ESI-pos) M+1=885.2; ¹H NMR (400 MHz, DMSO) δ 11.53 (s, 1H), 8.15 (s, 1H), 8.09-7.87 (m, 1H), 7.85-7.73 (m, 1H), 7.55-7.50 (m, 3H), 7.41 (d, J=8.4 Hz, 1H), 7.30 (d, J=7.2 Hz, 1H), 7.24 (d, J=8.4 Hz, 1H), 7.10 (s, 1H), 6.94-6.80 (m, 2H), 6.66-6.57 (m, 1H), 5.72-5.56 (m, 1H), 4.73-4.57 (m, 2H), 4.48-4.23 (m, 3H), 3.73 (d, J=8.0 Hz, 2H), 3.62-3.53 (m, 1H), 3.26 (s, 3H), 2.99-2.87 (m, 2H), 1.70-1.49 (m, 10H), 1.32-1.24 (m, 4H), 1.18 (s, 6H).

### Example 45—Synthesis of Compound 45

### Synthetic Scheme

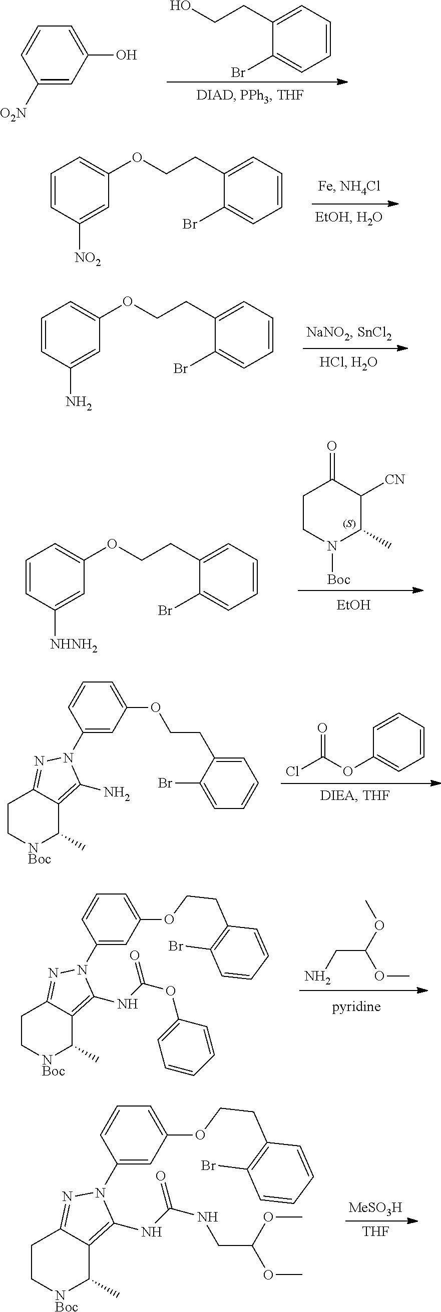

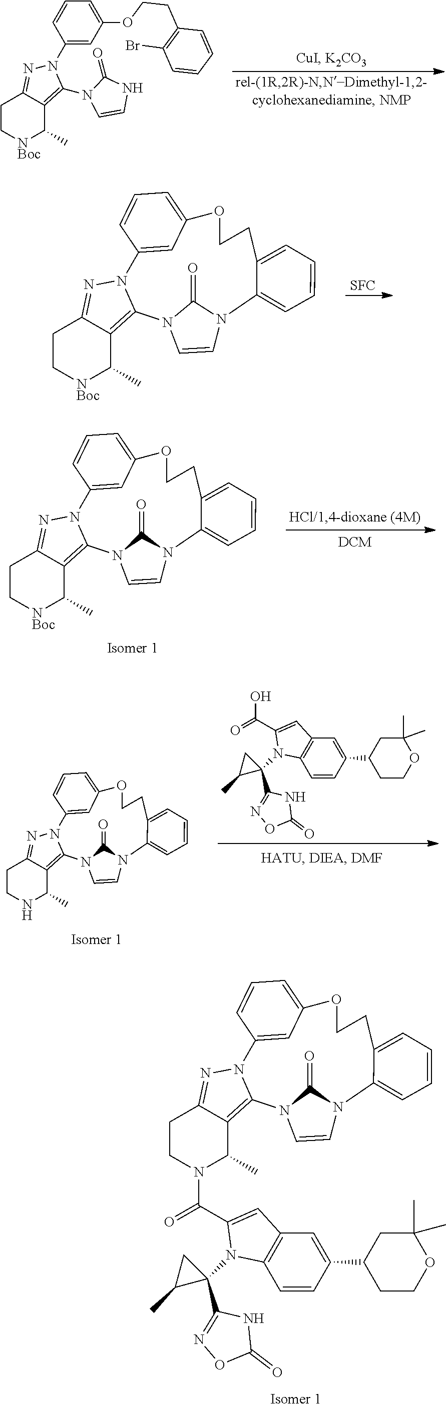

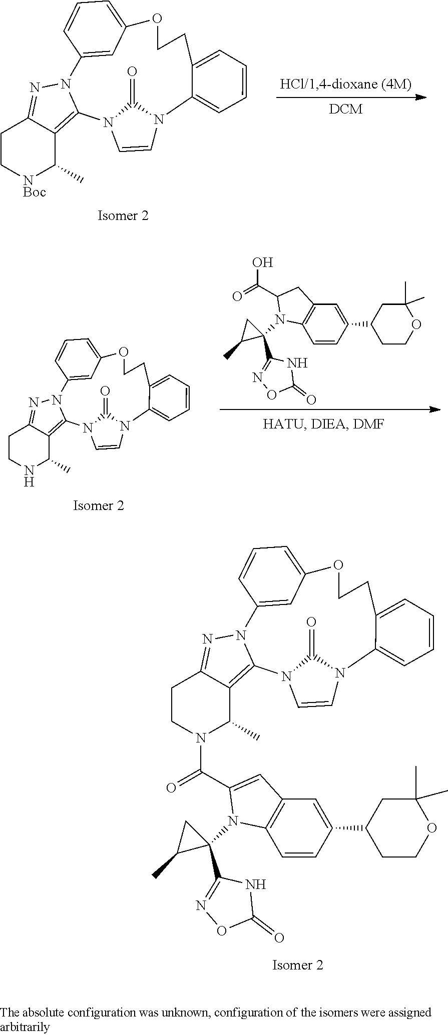

### Procedure for Synthesis of Compound 45

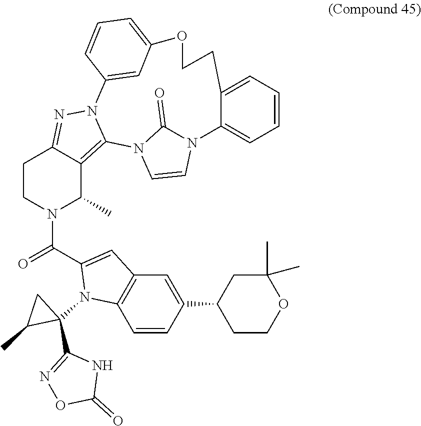

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[22.3.1.1¹¹,¹⁴.0²,¹⁰.0⁴,⁹.0¹⁵,²⁰]nonacosa-1(28), 3,9,12,15,17,19,24,26-nonaen-29-one

Step A: To a solution of m-nitrophenol (3 g, 21.6 mmol) in THF (60 mL) were added 2-(2-bromophenyl) ethan-1-ol (4.34 g, 21.6 mmol) and PPh3 (8.48 g, 32.3 mmol) at 0° C. under N₂, followed by the addition of DIAD (6.54 g, 32.3 mmol) dropwise. The reaction mixture was then wared to ambient temperature and stirred for 16 hours. The mixture was diluted with H₂O and extracted with EtOAc (2×). The combined EtOAc layers were washed with brine, dried over Na₂SO₄, filtered and concentrated in vacuo. The crude product was purified by column chromatography (0-10% EtOAc in PE) to give 1-bromo-2-(2-(3-nitrophenoxy)ethyl)benzene (4.3 g, 12 mmol) as a white solid. ¹HNMR (400 MHz, CDCl3) δ 7.81-7.78 (m, 1H), 7.73 (t, J=2.4 Hz, 1H), 7.58-7.56 (m, 1H), 7.40 (t, J=8.0 Hz, 1H), 7.35-7.33 (m, 1H), 7.30-7.26 (m, 1H), 7.23-7.20 (m, 1H), 7.14-7.10 (m, 1H), 4.26 (t, J=6.8 Hz, 2H), 3.27 (t, J=7.2 Hz, 2H).

Step B: To a solution of 1-bromo-2-(2-(3-nitrophenoxy)ethyl)benzene (4.3 g, 12 mmol) in EtOH (90 mL) was added iron (3.35 g, 60.1 mmol), ammonium chloride (3.21 g, 60.1 mmol) and water (30 mL). The mixture was stirred at 70° C. for 4 hours and then cooled to ambient temperature, diluted with brine and extracted with EtOAc (2×). The combined EtOAc layers were dried over Na₂SO₄, filtered and concentrated in vacuo. The crude product was purified by column chromatography (0-20% EtOAc in PE) to give 3-(2-bromophenethoxy) aniline (3.5 g, 10.8 mmol) as a white solid. m/z (ESI-pos) M+1=292.0, 294.0.

Step C: To a solution of 3-(2-bromophenethoxy) aniline (3.5 g, 10.8 mmol) in aq. HCl (6N., 60 mL) at 0° C. was added sodium nitrite (818 mg, 11.9 mmol) in H₂O (5 mL) dropwise. After the addition, the mixture was stirred at 0° C. for 45 mins, then SnCl₂ (4.5 g, 23.7 mmol) in aq. HCl (6N, 20 mL) was added dropwise. The mixture was stirred at ambient temperature for another 1 hour. The reaction was quenched with aq. NaOH (10N, 50 mL) to adjust pH to about 10. The mixture was extracted with EtOAc (3×), the combined organic layers were dried over anhydrous Na₂SO₄, filtered and the filtrate was concentrated under vacuum. The crude product was purified by column chromatography (0-50% EtOAc in PE) to give (3-(2-bromophenethoxy)phenyl)hydrazine (1.39 g, 4.07 mmol) as a yellow solid. m/z (ESI-pos) M+1=307.0, 309.0.

Step D: To a solution of (3-(2-bromophenethoxy)phenyl)hydrazine (1.39 g, 4.07 mmol) in EtOH (50 mL) was added tert-butyl(2S)-3-cyano-2-methyl-4-oxo-1-piperidinecarboxylate (970 mg, 4.07 mmol) and the mixture was stirred at 80° C. for 16 hours and then directly concentrated under reduced pressure. The crude product was purified by column chromatography (0-33% EtOAc in PE) to give tert-butyl (S)-3-amino-2-(3-(2-bromophenethoxy)phenyl)-4-methyl-2,4,6,7-tetrahydro-5H-pyrazolo[4,3-c]pyridine-5-carboxylate (1.6 g, 2.73 mmol) as a yellow solid. m/z (ESI-pos) M+1=527.1, 529.1.

Step E: To a solution of tert-butyl (S)-3-amino-2-(3-(2-bromophenethoxy)phenyl)-4-methyl-2,4,6,7-tetrahydro-5H-pyrazolo[4,3-c]pyridine-5-carboxylate (1.6 g, 2.7 mmol) in THF (30 mL) at 0° C. were added DIEA (1.06 g, 8.19 mmol) and benzenyl chloroformate (855 mg, 5.46 mmol). The mixture was stirred at ambient temperature for 2 hours. The mixture was concentrated under vacuum to get crude tert-butyl (S)-2-{m-[2-(o-bromophenyl)ethoxy]phenyl}-4-methyl-3-[phenyl(oxycarbonylamino)]-1,2,5-triaza-2,4,6,7-tetrahydro-5H-indene-5-carboxylate (2.5 g, 2.7 mmol) as a light yellow solid, which was used directly in next step without further purification. m/z (ESI-pos) M+1=647.1, 649.1.

Step F: To a solution of tert-butyl (S)-2-{m-[2-(o-bromophenyl)ethoxy]phenyl}-4-methyl-3-[phenyl(oxycarbonylamino)]-1,2,5-triaza-2,4,6,7-tetrahydro-5H-indene-5-carboxylate (2.5 g, 2.7 mmol) in pyridine (25 mL) was added 2,2-dimethoxyethylamine (852 mg, 8.11 mmol) and the mixture was stirred at ambient temperature for 2 hours. The reaction mixture was concentrated, and the crude product was purified by column chromatography (0-80% EtOAc in PE) to give tert-butyl (S)-2-(3-(2-bromophenethoxy)phenyl)-3-(3-(2,2-dimethoxyethyl)ureido)-4-methyl-2,4,6,7-tetrahydro-5H-pyrazolo[4,3-c]pyridine-5-carboxylate (1.2 g, 1.64 mmol) as a yellow solid. m/z (ESI-pos) M+1=658.2, 660.2.

Step G: To a solution of tert-butyl (S)-2-(3-(2-bromophenethoxy)phenyl)-3-(3-(2,2-dimethoxyethyl)ureido)-4-methyl-2,4,6,7-tetrahydro-5H-pyrazolo[4,3-c]pyridine-5-carboxylate (1.2 g, 1.82 mmol) in THF (80 mL) was added methanesulfonic acid (263 mg, 2.73 mmol) and the mixture was stirred at 60° C. for 2 hours. The reaction mixture was cooled to ambient temperature and diluted with water and then extracted with EtOAc (2×). The combined organic layers were dried over anhydrous Na₂SO₄, filtered and concentrated. The crude product was purified by column chromatography (0-100% EtOAc in PE) to give tert-butyl(S)-2-(3-(2-bromophenethoxy)phenyl)-4-methyl-3-(2-oxo-2,3-dihydro-1H-imidazol-1-yl)-2,4,6,7-tetrahydro-5H-pyrazolo[4,3-c]pyridine-5-carboxylate (0.8 g, 1.21 mmol) as a white solid. m/z (ESI-pos) M+1=594.2, 596.2.

Step H: To a solution of tert-butyl (S)-2-(3-(2-bromophenethoxy)phenyl)-4-methyl-3-(2-oxo-2,3-dihydro-1H-imidazol-1-yl)-2,4,6,7-tetrahydro-5H-pyrazolo[4,3-c]pyridine-5-carboxylate (770 mg, 1.17 mmol) in NMP (110 mL) was added CuI (222 mg, 1.17 mmol), (1R,2R)-1,2-bis(methylamino)cyclohexane (332 mg, 2.33 mmol) and K₂CO₃ (483 mg, 3.5 mmol). The mixture was stirred at 100° C. for 16 hours and then cooled to ambient temperature and diluted with H₂O. The mixture was extracted with EtOAc (2×) and the combined organics were washed with brine (2×), aq. LiCl, then dried over Na₂SO₄, filtered and concentrated in vacuo. The crude material was purified by column chromatography (0-50% EtOAc in PE). The product was then separated by chiral supercritical fluid chromatography [Column: Daicel CHIRALCEL OX, 250 mm*30 mm I.D., 10 μm; Mobile Phase: CO2/MeOH [0.2% NH₃ (7M Solution in MeOH)]=50/50; Flow rate: 120 g/min; Temp: 35° C.; Wavelength: 214 nm] to afford tert-butyl (S)-8-methyl-29-oxo-23-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate Isomer 1 (50 mg, 92.5 μmol) and Isomer 2 (110 mg, 203 μmol) as white solids (The absolute configurations were unknown and assigned arbitrarily). Isomer 1 m/z (ESI-pos) M+1=514.2; Isomer 2 m/z (ESI-pos) M+1=514.2.

Step I: To a solution of tert-butyl (S)-8-methyl-29-oxo-23-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate Isomer 2 (110 mg, 203 μmol) in DCM (5 mL) was added HCl/1,4-dioxane (4M, 5 mL) at 0° C. and then the mixture was stirred ambient temperature for 1 hour. The mixture was concentrated to give (S)-8-methyl-23-oxa-2,3,7,11,14 pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrochloride (Isomer 2, 88 mg, 202 μmol) as a white solid. m/z (ESI-pos) M+1=414.1.

Step J: To a solution of(S)-8-methyl-23-oxa-2,3,7,11,14 pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrochloride (88 mg, 202 μmol) in DMF (10 mL) was added 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (83.2 mg, 202 μmol) and DIEA (105 mg, 809 μmol). The mixture was stirred at ambient temperature for 10 mins and then HATU (99.9 mg, 263 μmol) was added and the mixture was stirred for 16 hours. The mixture was diluted with H₂O, and extracted with EtOAc (2×). The combined EtOAc layers were washed with brine, dried over Na₂SO₄, filtered and concentrated. The crude product was purified by column chromatography (0-5% MeOH in DCM). The product was further purified prep-HPLC [Instrument: Waters MS-triggered Prep-LC with QDA detector; Column: WELCH Xtimate C18 21.2*250 mm 10 μm; Gradient: eluted with 65-95% ACN in H2O (0.1% FA) in 9 min; Flow rate: 25 mL/min] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 2, 62.7 mg, 77.7 μmol) as a white solid. m/z (ESI-pos) M+1=807.3; ¹ HNMR (400 MHz, DMSO) δ 11.64 (s, 1H), 7.52-7.41 (m, 7H), 7.25 (d, J=7.6 Hz, 2H), 7.09 (s, 1H), 6.92-6.86 (m, 2H), 6.71 (s, 2H), 5.61 (s, 1H), 4.44 (s, 1H), 4.30-4.13 (m, 2H), 3.72 (d, J=7.6 Hz, 2H), 3.59 (s, 1H), 3.04-2.98 (m, 2H), 2.93-2.84 (m, 2H), 2.63-2.57 (m, 1H), 1.73-1.69 (m, 4H), 1.63-1.50 (m, 5H), 1.28 (s, 4H), 1.19 (s, 6H).

Step K: Steps I and J were repeated, with (Isomer 1, 50 mg) to afford(S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 1, 33.7 mg, 41.8 μmol) as a white solid. m/z (ESI-pos) M+1=807.3; ¹HNMR (400 MHz, DMSO) δ 11.61 (s, 1H), 7.53-7.39 (m, 7H), 7.25-7.22 (m, 2H), 7.03 (s, 1H), 6.93-6.90 (m, 1H), 6.87 (s, 1H), 6.74 (d, J=2.8 Hz, 1H), 6.68 (d, J=2.8 Hz, 1H), 5.65 (s, 1H), 4.42 (s, 1H), 4.31-4.23 (m, 1H), 4.12-4.06 (m, 1H), 3.73-3.71 (m, 2H), 3.58 (s, 1H), 3.02-2.98 (m, 2H), 2.90-2.79 (m, 2H), 2.65-2.59 (m, 1H), 1.71-1.68 (m, 3H), 1.65-1.50 (m, 6H), 1.28-1.25 (m, 4H), 1.19 (s, 6H).

### Example 46—Synthesis of Compound 46

### Synthetic Scheme

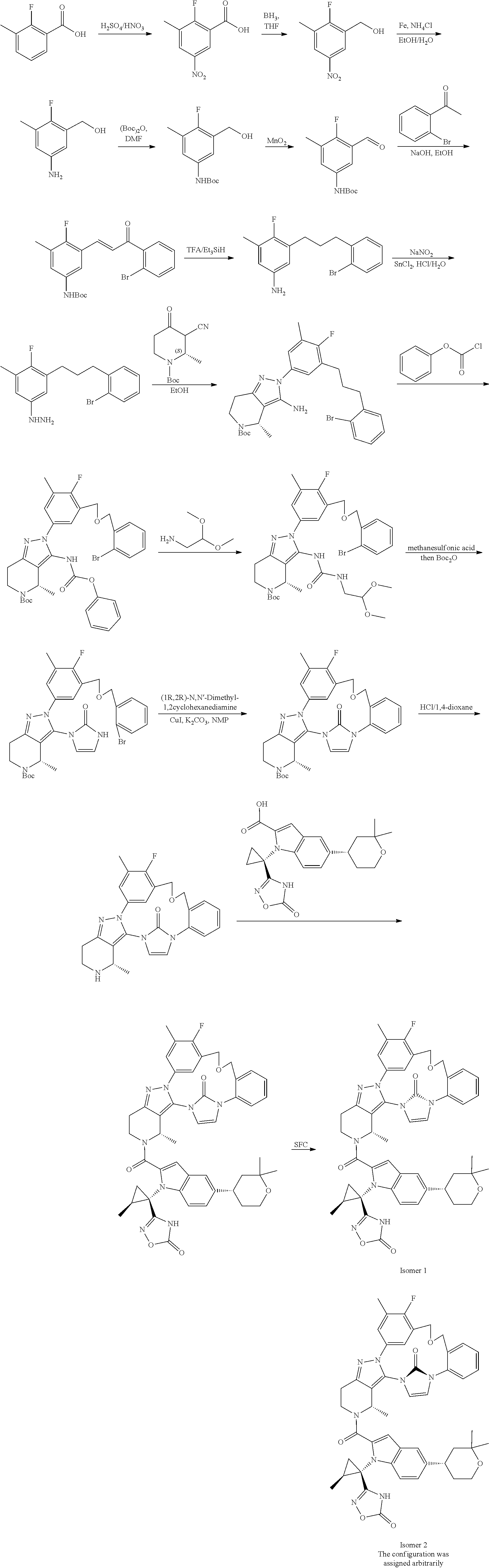

### Procedure for Synthesis of Compound 46

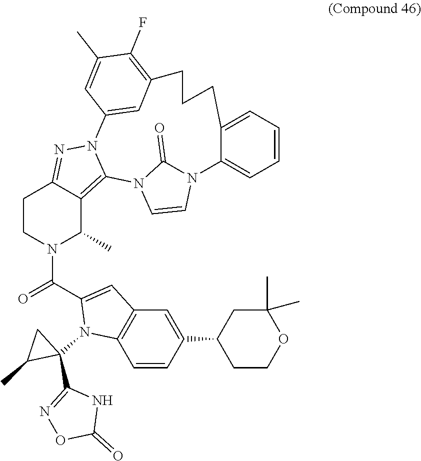

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of 2-fluoro-3-toluic acid (15 g, 97.3 mmol) in H₂SO₄ (90 mL) at 0° C. was added nitric acid (70% w/w, 13.1 g, 146 mmol) dropwise. The mixture was stirred at 0° C. for 3 hours. The mixture was poured into water and extracted with EtOAc (2×). The combined EtOAc layers were washed with brine (2×), dried over Na₂SO₄, filtered and the filtrate was concentrated in vacuo. The crude product was purified by column chromatography (0-50% EtOAc in PE) to give 2-fluoro-5-nitro-3-toluic acid (17 g, 76.8 mmol) as a white solid. m/z (ESI-pos) M+1=200.1.

Step B: To a solution of 2-fluoro-5-nitro-3-toluic acid (17 g, 76.8 mmol) in THF (340 mL) at 0° C. was added Borane-methyl sulfide complex (2M in THF, 115 mL, 230 mmol). The mixture was allowed to warm up to ambient temperature and stirred for 16 hours. The reaction mixture was carefully quenched with MeOH (80 mL) and then the mixture was stirred at 60° C. for 1 hour. Then the mixture was concentrated under reduced pressure and the residue was diluted with water. The mixture was extracted with EtOAc (2×) and the combined EtOAc layers were washed with brine (2×), dried over Na₂SO₄, filtered and concentrated in vacuo. The crude product was purified by column chromatography (0-50% EtOAc in PE) to give (2-fluoro-5-nitro-3-tolyl)methanol (14.5 g, 70.5 mmol) as a white solid. m/z (ESI-pos) M+1=186.2.

Step C: To a solution of (2-fluoro-5-nitro-3-tolyl)methanol (14.5 g, 78.3 mmol) in ethanol (290 mL) and water (116 mL) was added iron (21.9 g, 392 mmol) and ammonium chloride (20.9 g, 392 mmol). The reaction mixture was stirred at 80° C. for 2 h. The reaction mixture was concentrated under reduced pressure; the residue was diluted with ethyl acetate and washed with water (2×). The aqueous layer was extracted with ethyl acetate (2×). The combined organic layers were washed with brine solution, dried over anhydrous Na₂SO₄, filtered and evaporated under reduced pressure to give the crude (5-amino-2-fluoro-3-tolyl)methanol (13 g, 83.8 mmol), which was used directly to next step. m/z (ESI-pos) M+1=156.2

Step D: To a solution of (5-amino-2-fluoro-3-tolyl)methanol (13 g, 83.8 mmol) in DMF (111 mL) was added tert-butoxycarbonyl-tert-butyl carbonate (36.6 g, 168 mmol) and the mixture stirred at 60° C. for 3 h. The reaction mixture was cooled to ambient temperature and diluted with ethyl acetate then washed with water (2×). The aqueous layer was extracted with ethyl acetate (2×). The combined organic layers were washed with 1N aq. HCl and brine solution, dried over anhydrous Na₂SO₄, filtered and the filtrate was evaporated under reduced pressure. The crude compound was purified by column chromatography (0-80% EtOAc in PE) to give 4-fluoro-3-(hydroxymethyl)-5-tolyl 2-methyl-2-propanecarbamate (14.7 g, 57.6 mmol) as yellow oil. m/z (ESI-pos) M+23=278.1

Step E: To a solution of 4-fluoro-3-(hydroxymethyl)-5-tolyl 2-methyl-2-propanecarbamate (15 g, 58.8 mmol) in 1,2-dichloroethane (0.3 L) was added manganese dioxide (51.1 g, 588 mmol). The mixture was stirred at 50° C. overnight and then cooled to ambient temperature. The mixture was filtered and the filter cake was flushed with DCM. The filtrate was evaporated under reduced pressure. The residue was purified by column chromatography (0-50% EtOAc in PE) to give 4-fluoro-3-formyl-5-tolyl 2-methyl-2-propanecarbamate (12.8 g, 50.5 mmol) as a white solid. m/z (ESI-pos) M-56=198.1.

Step F: To a solution of 1-(o-bromophenyl)-1-ethanone (6.68 g, 33.6 mmol) in ethanol (85 mL) was added sodium hydroxide (268 mg, 6.71 mmol). The mixture was stirred at 0° C. for 10 mins, then 4-fluoro-3-formyl-5-tolyl 2-methyl-2-propanecarbamate (8.5 g, 33.6 mmol) was added into. The mixture was stirred at ambient temperature for 2 h and then concentrated under reduced pressure. The residue was diluted with ethyl acetate and washed with water (2×) and the aqueous layer was extracted with ethyl acetate (2×). The combined organic layers were washed with brine solution, dried over anhydrous Na₂SO₄ and evaporated under reduced pressure to give the crude compound, which was purified by column chromatography (0-80% EtOAc in PE) to give 3-[(E)-2-(o-bromobenzoyl)-1-ethenyl]-4-fluoro-5-tolyl 2-methyl-2-propanecarbamate (10 g, 23 mmol) as a yellow oil. m/z (ESI-pos) M+1=434.1/436.1

Step G: To a solution of 3-[(E)-2-(o-bromobenzoyl)-1-ethenyl]-4-fluoro-5-tolyl 2-methyl-2-propanecarbamate (10 g, 23 mmol) in TFA (62.5 mL) was added triethylsilane (18.7 g, 161 mmol). The mixture was stirred at ambient temperature for 2 h. The reaction mixture was concentrated under reduced pressure. The residue was diluted with ethyl acetate (100 mL), quenched with aq. NaHCO₃ (100 mL) and the organic layer was further washed with water (2×) and the aqueous layer was extracted with ethyl acetate (2×). The combined organic layers were washed with brine solution, dried over anhydrous Na₂SO₄, filtered and evaporated under reduced pressure to give the crude intermediate [(E)-3-(3-(2-bromophenyl) prop-1-en-1-yl)-4-fluoro-5-methylaniline], which was dissolved THF (40 mL) and treated with tosylhydrazine (21.4 g, 115 mmol) and sodium acetate (9.44 g, 115 mmol). The mixture was stirred at 80° C. for 2 h. The reaction mixture was cooled to ambient temperature and diluted with ethyl acetate, washed with water (2×) and the aqueous layer was extracted with ethyl acetate (2×). The combined organic layers were washed with brine solution (2×), dried over anhydrous Na₂SO₄, filtered and evaporated under reduced pressure. The crude product was purified by column chromatography (0-50% EtOAc in PE) to give 3-[3-(o-bromophenyl)propyl]-4-fluoro-5-tolylamine (4.8 g, 14.9 mmol) as a yellow oil. m/z (ESI-pos) M+1=322.1

Step H: To a solution of 3-[3-(o-bromophenyl)propyl]-4-fluoro-5-tolylamine (4.8 g, 14.9 mmol) in 6M HCl (80 mL) at 0° C. was added a solution of NaNO₂ (1234 mg, 17.88 mmol) in water (15 mL) dropwise. The mixture was then stirred at 0° C. for 1 h. A solution of SnCl₂ (5.65 g, 29.8 mmol) in aq. HCl (6N, 20 mL) was added dropwise to the reaction mixture and then the mixture was allowed to warm to ambient temperature and stirred for 2 h. The mixture was diluted with ethyl acetate, washed with water (2×) and the aqueous layer was extracted with ethyl acetate (2×). The combined organic layers were washed with brine solution and dried over anhydrous Na₂SO₄, filtered and the filtrate was evaporated under reduced pressure to give the crude {3-[3-(o-bromophenyl)propyl]-4-fluoro-5-tolyl}hydrazine (3 g, 8.9 mmol) as yellow liquid, which was used immediately in the next step. m/z (ESI-pos) M+1=337.1/339.1

Step I: To a solution of crude {3-[3-(o-bromophenyl)propyl]-4-fluoro-5-tolyl}hydrazine (3 g, 8.9 mmol) in ethanol (60 mL) was added tert-butyl(2S)-3-cyano-2-methyl-4-oxo-1-piperidinecarboxylate (2.12 g, 8.9 mmol). The resulting mixture was stirred at 80° C. for 16 hr. The reaction mixture was concentrated under reduced pressure to give the crude compound, which was purified by column chromatography (0-60% EtOAc in PE) to give tert-butyl (S)-3-amino-2-{3-[3-(o-bromophenyl)propyl]-4-fluoro-5-tolyl}-4-methyl-1,2,5-triaza-2,4,6,7-tetrahydro-5H-indene-5-carboxylate (2 g, 3.59 mmol) as yellow oil. m/z (ESI-pos) M+1=557.3/559.3

Step J: To a solution of tert-butyl (S)-3-amino-2-{3-[3-(o-bromophenyl)propyl]-4-fluoro-5-tolyl}-4-methyl-1,2,5-triaza-2,4,6,7-tetrahydro-5H-indene-5-carboxylate (2 g, 3.59 mmol) in THF (5.71 mL) was added benzenyl chloroformate (674 mg, 4.3 mmol). The reaction was stirred at 25° C. for 2 hr. The reaction mixture was concentrated to give crude tert-butyl (S)-2-{3-[3-(o-bromophenyl)propyl]-4-fluoro-5-tolyl}-4-methyl-3-[phenyl(oxycarbonylamino)]-1,2,5-triaza-2,4,6,7-tetrahydro-5H-indene-5-carboxylate (2.2 g, 3.25 mmol) as yellow oil. m/z (ESI-pos) M+1=677.2.

Step K: Crude tert-butyl (S)-2-{3-[3-(o-bromophenyl)propyl]-4-fluoro-5-tolyl}-4-methyl-3-[phenyl(oxycarbonylamino)]-1,2,5-triaza-2,4,6,7-tetrahydro-5H-indene-5-carboxylate (2.2 g, 3.25 mmol) was dissolved in pyridine (17.6 mL), to which was added 2,2-dimethoxyethylamine (1.02 g, 9.74 mmol). The mixture was stirred at 25° C. for 3 hr. The reaction mixture was concentrated under reduced pressure. The crude compound was purified by column chromatography (0-80% EtOAc in PE) to give tert-butyl (S)-2-{3-[3-(o-bromophenyl)propyl]-4-fluoro-5-tolyl}-3-[3-(2,2-dimethoxyethyl)ureido]-4-methyl-1,2,5-triaza-2,4,6,7-tetrahydro-5H-indene-5-carboxylate (2 g, 2.9 mmol) as yellow solid. m/z (ESI-pos) M+1=688.3/690.3

Step L: To a solution of tert-butyl (S)-2-{3-[3-(o-bromophenyl)propyl]-4-fluoro-5-tolyl}-3-[3-(2,2-dimethoxyethyl)ureido]-4-methyl-1,2,5-triaza-2,4,6,7-tetrahydro-5H-indene-5-carboxylate (2 g, 2.9 mmol) in THF (20 mL) was added methanesulfonic acid (279 mg, 2.9 mmol). The mixture was stirred at 60° C. for 2 h. Then aqueous K₃PO₄ was added to the reaction mixture until pH 8-9, and then tert-butyl-tert-butyl (oxycarbonyloxy) formylate (127 mg, 581 μmol) was added. The resulting mixture was stirred at ambient temperature for 1 h, and then the mixture was diluted with ethyl acetate, washed with water (2×) and the aqueous layer was extracted with ethyl acetate (2×). The combined organic layers were washed with IN aq. HCl (20 mL) and the organic layers were washed with brine and then dried over anhydrous Na₂SO₄, filtered and the filtrate was evaporated under reduced pressure to give the crude product, which was purified by column chromatography (0-80% EtOAc in PE) to give product tert-butyl (S)-2-{3-[3-(o-bromophenyl)propyl]-4-fluoro-5-tolyl}-4-methyl-3-(2-oxo-2,3-dihydro-1H-imidazol-1-yl)-1,2,5-triaza-2,4,6,7-tetrahydro-5H-indene-5-carboxylate (1.6 g, 2.56 mmol) as yellow oil. m/z (ESI-pos) M+1=624.2/626.2.

Step M: A mixture of tert-butyl (S)-2-{3-[3-(o-bromophenyl)propyl]-4-fluoro-5-tolyl}-4-methyl-3-(2-oxo-2,3-dihydro-1H-imidazol-1-yl)-1,2,5-triaza-2,4,6,7-tetrahydro-5H-indene-5-carboxylate (1.6 g, 2.56 mmol), copper iodide (488 mg, 2.56 mmol), (1R,2R)-1,2-bis(methylamino)cyclohexane (729 mg, 5.12 mmol) and K₂CO₃ (1.06 g, 7.69 mmol) in NMP (240 mL) was stirred at 130° C. for 4 h. The reaction mixture was cooled to ambient temperature and diluted with ethyl acetate then washed with water (2×). The aqueous layer was extracted with ethyl acetate (2×) and the combined organic layers were washed with aq. LiCl solution, dried over anhydrous Na₂SO₄, filtered and the filtrate was evaporated under reduced pressure. The residue was purified by column chromatography (0-80% EtOAc in PE) to give tert-butyl (S)-25-fluoro-8,26-dimethyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (120 mg, 221 μmol) as a white solid. m/z (ESI-pos) M+1=544.2

Step N: tert-butyl (S)-25-fluoro-8,26-dimethyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (120 mg, 221 μmol) was dissolved in HCl/1,4-dioxane (4M, 2 mL) and stirred at 25° C. for 1 h. The reaction mixture was directly concentrated to give (S)-25-fluoro-8,26-dimethyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-on hydrogen chloride (110 mg, 247 μmol) as a yellow solid, which was used directly in the next step. m/z (ESI-pos) M+1=444.2

Step O: To a solution of 5-[(S)-2,2-dimethyltetrahydro-4H-pyran-4-yl]-1-[(2S)-2-methyl-1-(5-oxo-4,5-dihydro-1,2,4-oxadiazol-3-yl)cyclopropyl]-2-indolecarboxylic acid (102 mg, 247 μmol), in DMF (11 mL) was added DIEA (95.9 mg, 742 μmol) and HATU (113 mg, 297 μmol), the mixture stirred at ambient temperature for 10 min, then(S)-25-fluoro-8,26-dimethyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-on hydrogen chloride (110 mg, 247 μmol) was added to the mixture. The mixture was stirred at ambient temperature overnight. The reaction mixture was diluted with ethyl acetate, and washed with water (2×). The aqueous layer was extracted with ethyl acetate (2×). The combined organic layers were washed with brine, dried over anhydrous Na₂SO₄, filtered and the filtrate was evaporated under reduced pressure. The residue was purified by column chromatography (0-80% EtOAc in PE) to give product, which was further purified by prep-HPLC [Instrument: Waters Prep-LC with UV detector; Column: Waters Luna C18 10 μm 21.2*250 mm; Gradient: eluted with 70-80% acetonitrile in H₂O (0.1% FA) in 12 min; Flow rate: 20 mL/min to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (80 mg, 95.6 μmol) as a white solid. The atropisomer mixture was separated by SFC [Instrument: SFC 150; Column: Daicel CHIRALPAK IB-N, 250 mm*30 mm I.D., 10 μm; Mobile phase: CO2/MeOH [0.2% NH₃ (7M Solution in MeOH)]=50/50; Flow rate: 120 g/min; Wave length: UV 214 nm; Temperature: 35° C.] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 1, 31.6 mg) and (Isomer 2, 19.2 mg) as white solids (The configurations were unknown and assigned arbitrarily). m/z (ESI-pos) M+1=837.3; ¹H NMR (400 MHz, DMSO) (Isomer 1) δ 11.69 (s, 1H), 7.55-748 (m, 1H), 7.46-7.29 (m, 6H), 7.27-7.19 (m, 1H), 7.12-7.02 (m, 1H), 6.91-6.55 (m, 3H), 5.60 (s, 1H), 4.50 (s, 1H), 3.77-3.68 (m, 2H), 3.67-3.46 (m, 1H), 3.00-2.85 (m, 3H), 2.84-2.67 (m, 2H), 2.40-2.25 (m, 4H), 2.19-2.07 (m, 1H), 2.06-1.95 (m, 1H), 1.94-1.83 (m, 2H), 1.78-1.67 (m, 3H), 1.66-1.46 (m, 6H), 1.26-1.18 (m, 9H);¹H NMR (400 MHz, DMSO) (Isomer 2) δ 11.59 (s, 1H), 7.57-7.27 (m, 7H), 7.26-7.20 (m, 1H), 7.10-7.01 (m, 1H), 6.91-6.75 (m, 1H), 6.62 (m, 2H), 5.58 (s, 1H), 4.44 (s, 1H), 3.76-3.68 (m, 2H), 3.66-3.51 (m, 1H), 3.01-2.67 (m, 4H), 2.41-2.29 (m, 4H), 2.20-2.09 (m, 1H), 2.05-1.83 (m, 3H), 1.78-1.46 (m, 10H), 1.27-1.24 (m, 9H).

### Example 47—Synthesis of Compound 47

### Synthetic Scheme

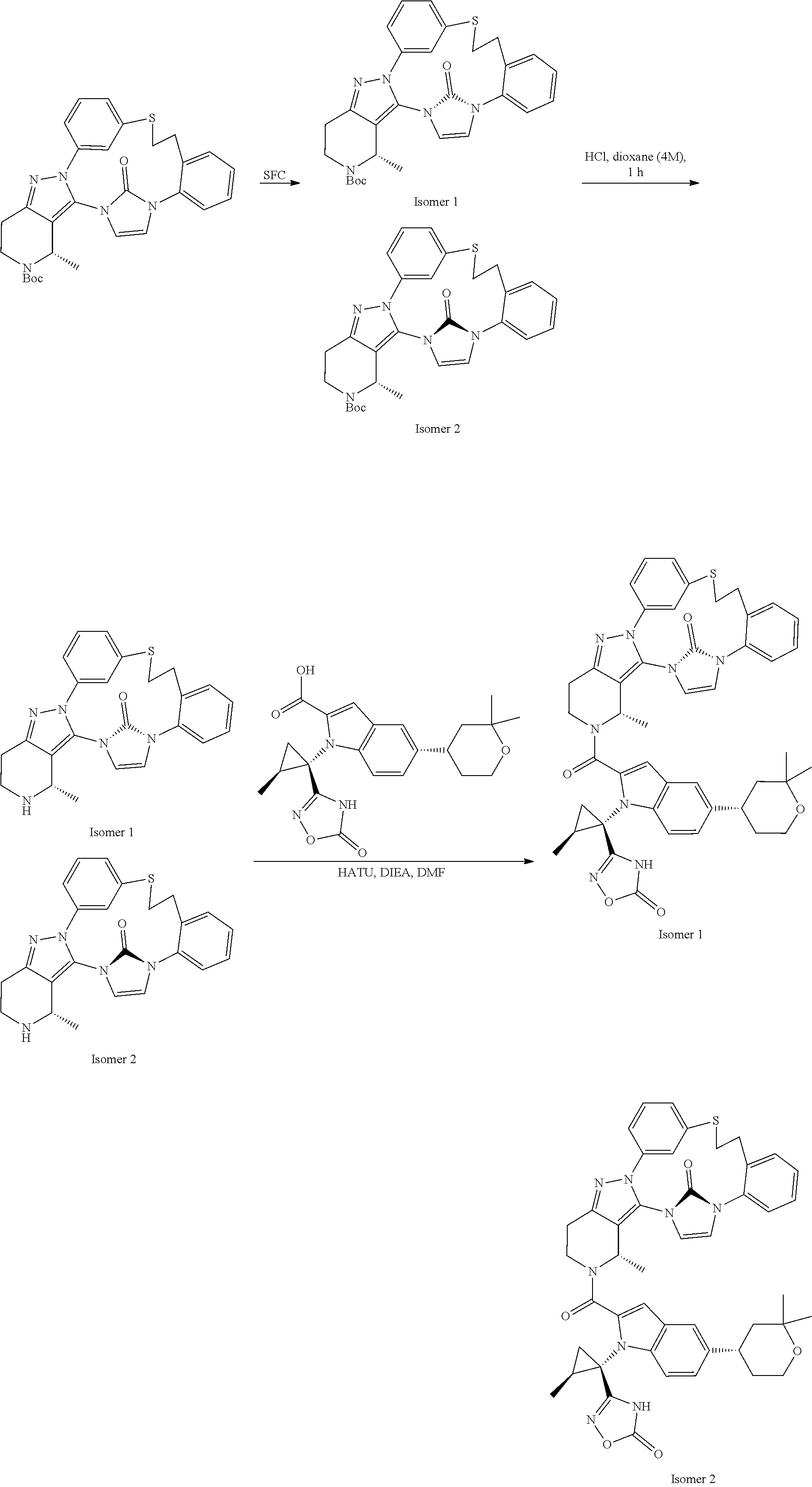

### Procedure for Synthesis of Compound 47

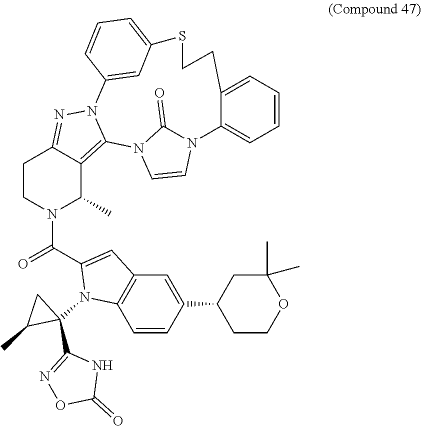

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-23-thia-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: tert-butyl (S)-8-methyl-29-oxo-23-thia-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate was prepared using the procedure described for Example 17, where 3-(o-bromophenyl)-1-propanol was replaced with 2-(o-bromophenyl) ethanol in Step A. The isomers were separated by SFC [Apparatus: Gilson281; Column: Daicel CHIRALCEL IC, 250 mm×30 mm I.D., 10 μm Mobile phase: Hex/EtOH [0.2% NH₃ (7M Solution in MeOH)]=40/60; Flow rate: 30 ml/min] to give Isomer 1 (400 mg) and Isomer 2 (190 mg). (The absolute configuration was unknown, the configurations of two atropisomers were assigned arbitrarily) m/z (ESI-pos) M+1=530.2.

Step B: tert-butyl (S)-8-methyl-29-oxo-23-thia-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (Isomer 1, 150 mg) was dissolved in HCl/1,4-dioxane (4M, 2 mL) and stirred at 25° C. for 1 h. The reaction mixture was directly concentrated to give (S)-8-methyl-23-thia-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (120 mg, 279 μmol) as a yellow solid, which was used directly in the next step. m/z (ESI-pos) M+1=430.2

Step C: To a solution of(S)-8-methyl-23-thia-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (70 mg, 163 μmol) in DMF (2.33 mL) was added DIEA (63.2 mg, 489 μmol) and HATU (124 mg, 326 μmol). The mixture stirred at ambient temperature for 10 min, then 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (80.5 mg, 196 μmol) was added and the mixture was stirred overnight. The reaction mixture was diluted with ethyl acetate, washed with water (2×). The aqueous layer was extracted with ethyl acetate (2×). The combined organic layers were washed with brine, dried over anhydrous Na₂SO₄, filtered and the filtrate was evaporated under reduced pressure. The crude compound was purified by prep-HPLC [Instrument: Waters MS-triggered Prep-LC with SQD2 detector; Column: Welch Ultimate AQ-C18 10 μm 21.2*250 mm; Gradient: 60-90% ACN in H₂O (0.1% FA) in 9.5 min; Flow rate: 25 mL/min] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-23-thia-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 1, 47.3 mg) as a white solid. m/z (ESI-pos) M+1=823.2; ¹H NMR (400 MHz, DMSO) δ 11.50 (s, 1H), 7.53 (d, J=7.2 Hz, 2H), 7.50-7.28 (m, 8H), 7.25 (dd, J=8.4, 0.8 Hz, 1H), 6.86 (s, 1H), 6.75 (s, 2H), 5.60 (s, 1H), 4.45 (s, 1H), 3.75-3.69 (m, 2H), 3.59 (s, 1H), 3.23-3.14 (m, 1H), 3.04-2.81 (m, 5H), 2.61-2.51 (m, 1H), 1.94-1.45 (m, 10H), 1.32-1.16 (m, 9H).

Step D: Steps B and C were repeated replacing Isomer 1 with Isomer 2 to provide (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-23-thia-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 2, 63.5 mg) as a white solid. m/z (ESI-pos) M+1=823.2; ¹H NMR (400 MHz, DMSO) δ 11.03 (s, 1H), 7.58-7.35 (m, 8H), 7.33-7.19 (m, 3H), 6.88 (s, 1H), 6.71 (dd, J=19.2, 2.0 Hz, 2H), 5.61 (s, 1H), 4.43 (s, 1H), 3.76-3.69 (m, 2H), 3.59 (s, 1H), 3.17 (d, J=13.2 Hz, 1H), 3.03-2.78 (m, 5H), 2.61-2.54 (m, 1H), 1.85-1.48 (m, 10H), 1.32-1.13 (m, 9H).

### Example 48—Synthesis of Compound 48

### Synthetic Scheme

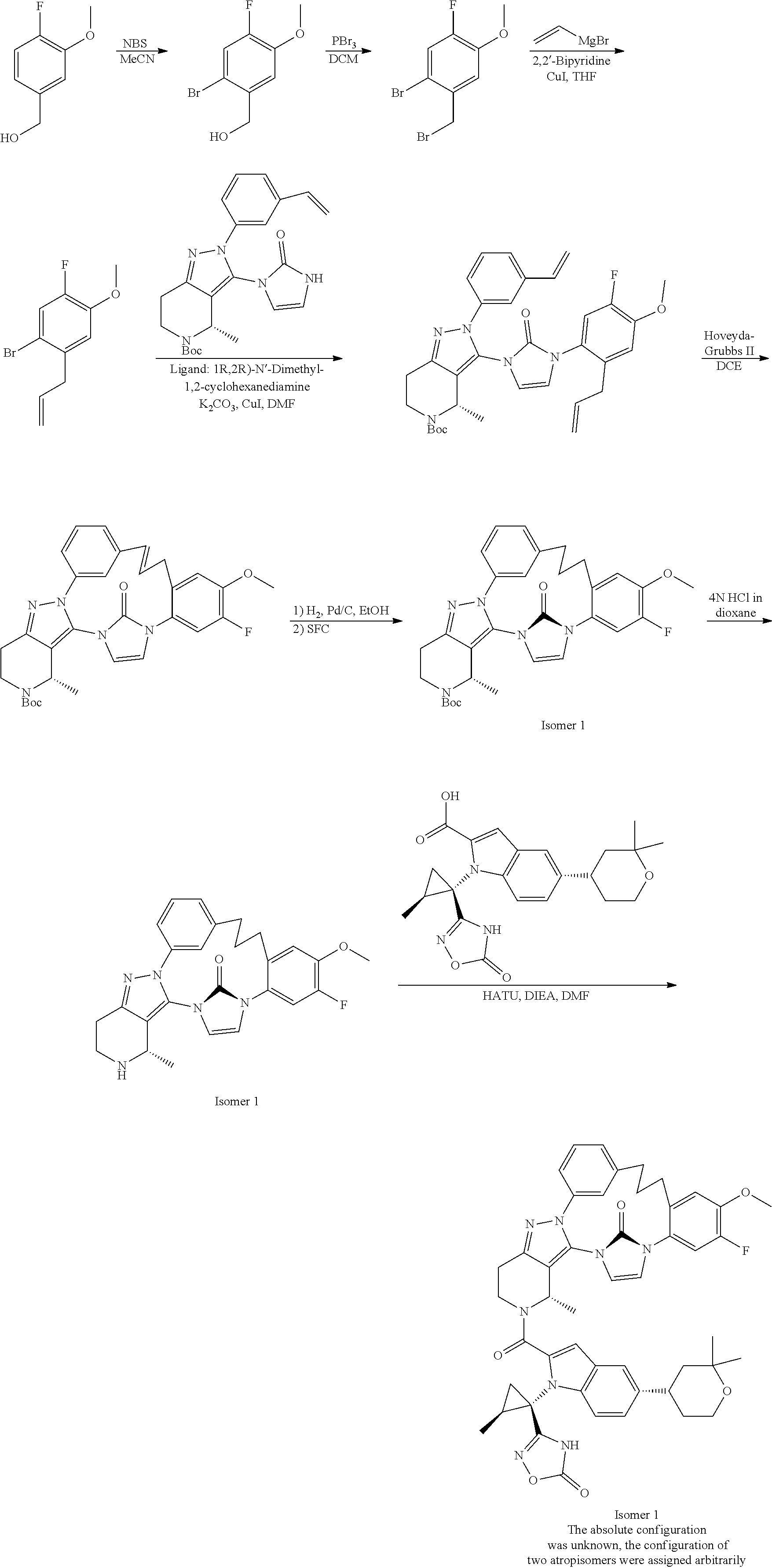

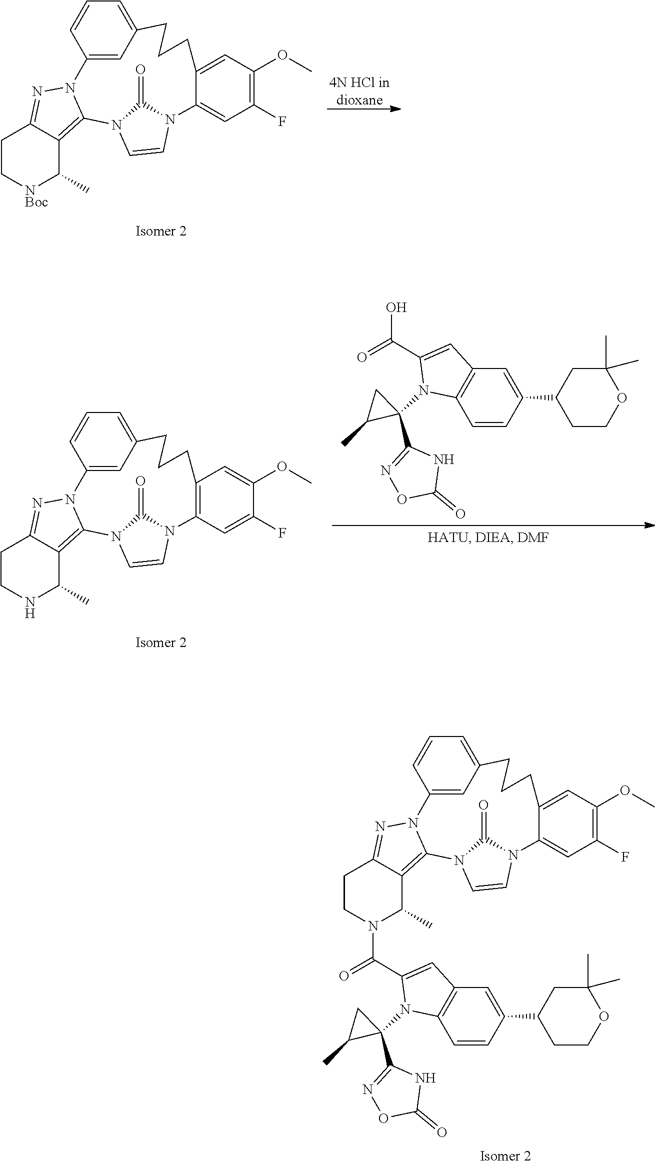

### Procedure for Synthesis of Compound 48

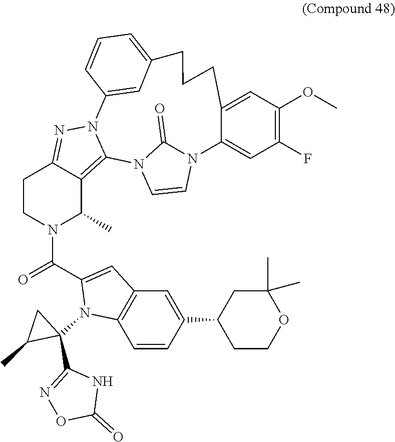

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17-fluoro-18-methoxy-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (4-fluoro-3-methoxyphenyl) methanol (5.0 g, 32.0 mmol) in acetonitrile (100 mL) was added 1-bromo-2,5-pyrrolidinedione (5.7 g, 32.0 mmol) at 25° C., the mixture was stirred for 16 h. The reaction mixture was concentrated in vacuo and the residue was suspended in diethyl ether. Solid material was removed by filtration and washed with further diethyl ether. The filtrate was washed with water and brine, then dried over Na₂SO₄, filtered and concentrated in vacuo to give (2-bromo-4-fluoro-5-methoxyphenyl) methanol (4.68 g, 17.9 mmol) as a white solid. ¹H NMR (400 MHz, CDCl₃) δ 7.27 (d, J=10.7 Hz, 1H), 7.15 (d, J=8.8 Hz, 1H), 4.70 (s, 2H), 3.90 (s, 3H).

Step B: To a solution of (2-bromo-4-fluoro-5-methoxyphenyl) methanol (4.68 g, 19.9 mmol) in dichloromethane (108 mL) was added tribromophosphane (10.8 g, 39.8 mmol) at 0° C., the mixture was then stirred at 25° C. for 16 h and then cooled to 0° C. again. The reaction was quenched by slow addition of saturated sodium hydrogen carbonate aqueous solution until effervescence had ceased. The layers were separated and the aqueous layer was extracted with DCM (2×). The combined organic layers were dried over MgSO₄, filtered and concentrated in vacuo to give 1-bromo-2-(bromomethyl)-5-fluoro-4-methoxybenzene (4.34 g, 13.8 mmol) as a white solid. ¹H NMR (400 MHz, DMSO) δ 7.61 (d, J=10.8 Hz, 1H), 7.53 (d, J=9.0 Hz, 1H), 4.70 (s, 2H), 3.85 (s, 3H).

Step C: To a mixture of 1-bromo-2-(bromomethyl)-5-fluoro-4-methoxybenzene (4.19 g, 14.1 mmol), 2,2′-bipyridyl (439 mg, 2.81 mmol), copper iodide (536 mg, 2.81 mmol) in THF (223 mL) at 0° C. was added vinylmagnesium bromide (1.0M in THF, 14.1 mL, 14.1 mmol) under N₂ atmosphere. The reaction mixture was stirred at 0° C. for 1 hour and was then quenched with saturated aqueous NH₄Cl solution, extracted with DCM (2×). The combined organics were dried over Na₂SO₄, filtered and the filtrate was concentrated under reduced pressure. The crude residue was purified by column chromatography (0-50% EtOAc in PE) to give 1-allyl-2-bromo-4-fluoro-5-methoxybenzene (2.09 g, 5.97 mmol) as a yellow oil. ¹H NMR (400 MHz, CDCl₃) δ 7.28 (d, J=10.8 Hz, 1H), 6.82 (d, J=8.8 Hz, 1H), 5.96-5.89 (m, 1H), 5.14-5.05 (m, 1H), 3.86 (s, 3H), 3.45 (d, J=6.4 Hz, 2H).

Step D: To a mixture of tert-butyl (S)-4-methyl-3-(2-oxo-2,3-dihydro-1H-imidazol-1-yl)-2-(m-styrenyl)-1,2,5-triaza-2,4,6,7-tetrahydro-5H-indene-5-carboxylate (2.23 g, 5.29 mmol), 1-allyl-2-bromo-4-fluoro-5-methoxybenzene (2.07 g, 8.46 mmol) in NMP (68.6 mL) was added K₂CO₃ (2.19 g, 15.9 mmol), (1R,2R)-1,2-bis(methylamino)cyclohexane (1.13 g, 7.94 mmol) and CuI (1.01 g, 5.29 mmol). The mixture was stirred at 130° C. under N₂ atmosphere for 4 h and then cooled to ambient temperature. The mixture was treated with water, then extracted with EtOAc (2×). The combined organics were dried over Na₂SO₄, filtered and the filtrate was concentrated under reduced pressure. The crude residue was purified by column chromatography (0-80% EtOAc in PE) to give tert-butyl (S)-3-[3-(2-allyl-5-fluoro-4-methoxyphenyl)-2-oxo-2,3-dihydro-1H-imidazol-1-yl]-4-methyl-2-(m-styrenyl)-1,2,5-triaza-2,4,6,7-tetrahydro-5H-indene-5-carboxylate (1.18 g, 1.81 mmol) as a yellow solid. m/z (ESI-pos) M+1=586.2.

Step E: To a solution of tert-butyl (S)-3-[3-(2-allyl-5-fluoro-4-methoxyphenyl)-2-oxo-2,3-dihydro-1H-imidazol-1-yl]-4-methyl-2-(m-styrenyl)-1,2,5-triaza-2,4,6,7-tetrahydro-5H-indene-5-carboxylate (1.18 g, 2.01 mmol) in 1,2-dichloroethane (1.18 L) at 25° C. was added Hoveyda Grubbs II (1.18 g, 1.89 mmol) and the mixture solution was stirred at 80° C. for 3.5 hours. The mixture was concentrated and the crude compound was purified by column chromatography (0-100% EtOAc in PE) to give tert-butyl (S)-17-fluoro-18-methoxy-8-methyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,22,24,26-decaene-7-carboxylate (780 mg, 1.26 mmol) as a yellow solid. m/z (ESI-pos) M+1=558.2.

Step F: To a solution of tert-butyl (S)-17-fluoro-18-methoxy-8-methyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,22,24,26-decaene-7-carboxylate (780 mg, 1.26 mmol) in ethanol (14 mL) was added Pd/C (375 mg). The mixture was then evacuated and backfilled with H₂. The mixture was stirred at ambient temperature under H₂ balloon for 16 h and was then diluted with ethanol and filtered through a pad of celite. The filtrate was evaporated under reduced pressure to give tert-butyl (S)-17-fluoro-18-methoxy-8-methyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (718 mg, 1.15 mmol) as a yellow solid. The solid was then dissolved in MeOH (150 mL) and separated by chiral supercritical fluid chromatography [Apparatus: SFC 80; Column: Daicel CHIRALCEL IB-N, 250 mm×30 mm I.D., 10 μm; Mobile Phase: CO₂/MeOH [0.2% NH₃ (7M Solution in MeOH)]=65/35 at 80 g/min; Temp: 35° C.; Wavelength: 214 nm] to afford (Isomer 1, 336 mg) and (Isomer 2, 260 mg) as white solids. (The absolute configuration was unknown, the configuration of two atropisomers were assigned arbitrarily). m/z (ESI-pos) M+1=560.2.

Step G: tert-butyl (S)-17-fluoro-18-methoxy-8-methyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (Isomer 1, 60 mg, 107 μmol) was dissolved in 4 N HCl in 1,4-dioxane (4 mL) and stirred at ambient temperature for 1 h and was then directly concentrated under reduced pressure to give (S)-17-fluoro-18-methoxy-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (48 mg, 104 μmol) as yellow oil. m/z (ESI-pos) M+1=460.2

Step H: To a solution of(S)-17-fluoro-18-methoxy-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (Isomer 1, 48 mg, 104 μmol), 5-[(S)-2,2-dimethyltetrahydro-4H-pyran-4-yl]-1-[(2S)-2-methyl-1-(5-oxo-4,5-dihydro-1,2,4-oxadiazol-3-yl)cyclopropyl]-2-indolecarboxylic acid (44 mg, 107 μmol) in DMF (6 mL) was added DIEA (138 mg, 1.07 mmol) and HATU (61 mg, 160 μmol). The mixture was stirred at ambient temperature under N₂ atmosphere for 16 hours. The reaction mixture was diluted with ethyl acetate and washed with water (2×). The aqueous layer was extracted with ethyl acetate (2×). The combined organic layers were washed with brine, dried over anhydrous Na₂SO₄, filtered and the filtrate was evaporated under reduced pressure to give the crude compound and purified by column chromatography (0-10% MeOH in DCM) to give the product, which was further purified by pre-HPLC [Instrument: Waters Prep-LC with UV detector; Column: Waters Luna C18 10 μm 21.2*250 mm; Gradient: eluted with 70-95% ACN in H₂O (0.1% FA) in 12.5 min; Flow rate: 20 mL/min] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17-fluoro-18-methoxy-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (26.4 mg, 30.9 μmol) (Isomer 1) as a white solid. m/z (ESI-pos) M+1=853.3; ¹H NMR (400 MHz, DMSO) δ 11.63 (s, 1H), 7.51 (s, 1H), 7.44-7.39 (m, 3H), 7.28-7.14 (m, 5H), 6.92-6.75 (m, 1H), 6.67-6.45 (m, 2H), 5.70-5.48 (m, 1H), 4.57-4.33 (m, 1H), 3.89 (s, 3H), 3.73-3.71 (m, 2H), 3.62-3.45 (m, 1H), 2.90-2.65 (m, 4H), 2.33-2.25 (m, 1H), 2.08-2.00 (m, 1H), 1.96-1.49 (m, 12H), 1.32-1.24 (m, 4H), 1.19 (s, 6H).

Step I: Steps G and H were repeated using Isomer 2 in place of Isomer 1 to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17-fluoro-18-methoxy-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 2, 34.2 mg, 40.0 μmol) as a white solid. m/z (ESI-pos) M+1=853.3; ¹H NMR (400 MHz, DMSO) δ 11.59 (s, 1H), 7.50 (s, 1H), 7.43-7.35 (m, 4H), 7.24-7.14 (m, 4H), 6.87 (s, 1H), 6.55-6.52 (m, 2H), 5.70-5.49 (m, 1H), 4.49-4.34 (m, 1H), 3.89 (s, 3H), 3.76-3.70 (m, 2H), 3.59-3.50 (m, 1H), 2.99-2.84 (m, 3H), 2.71-2.66 (m, 1H), 2.32-2.24 (m, 1H), 2.09-2.01 (m, 1H), 1.97-1.49 (m, 12H), 1.30-1.24 (m, 4H), 1.18 (s, 6H).

### Example 49—Synthesis of Compound 49

### Synthetic Scheme

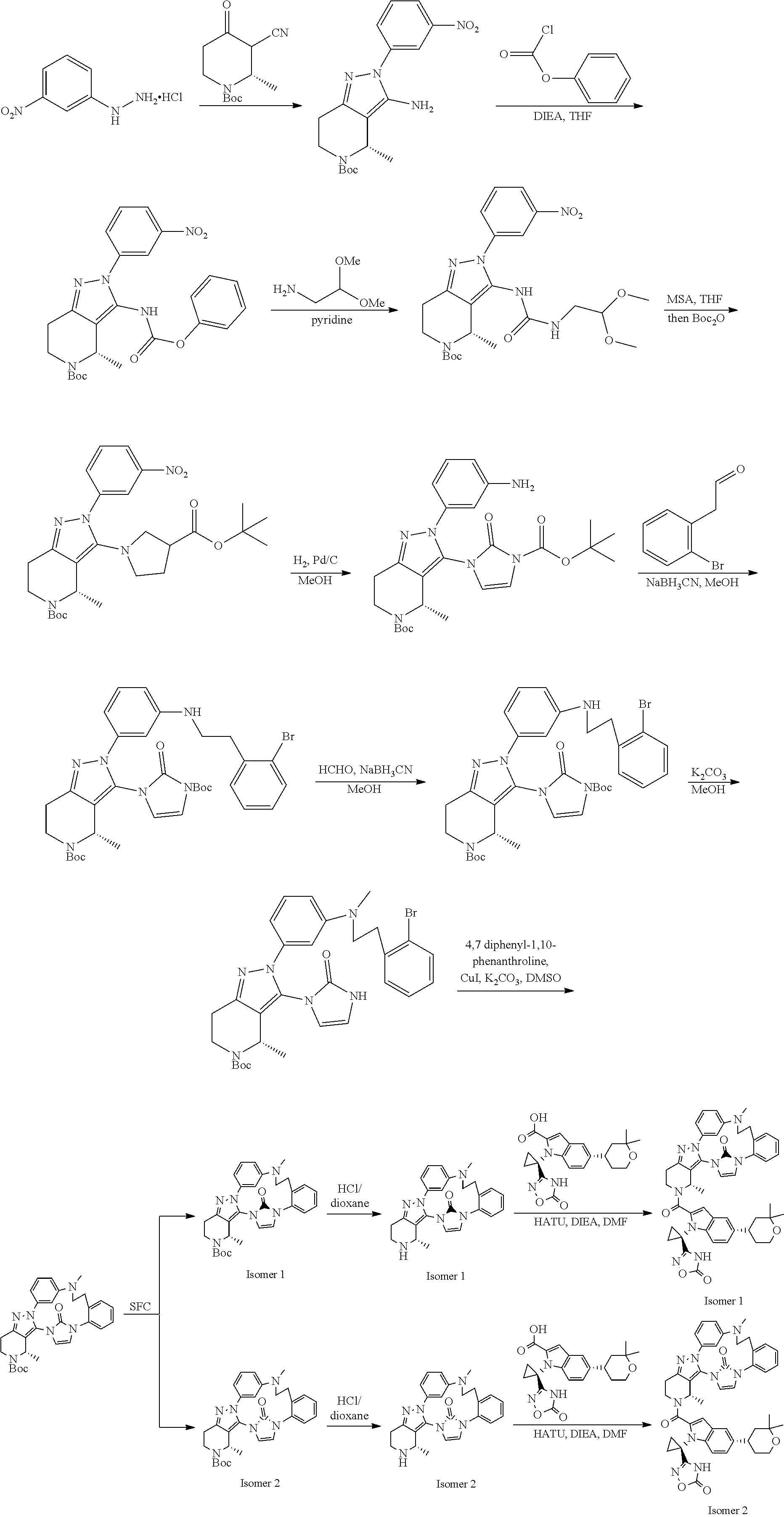

### Procedure for Synthesis of Compound 49

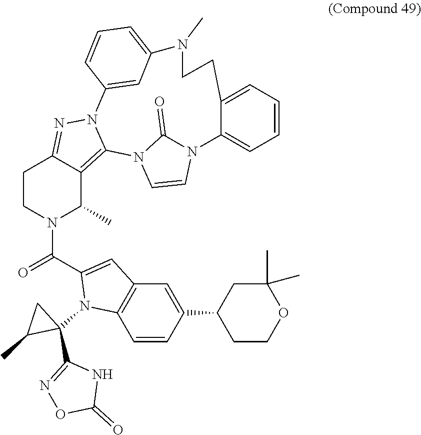

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-23-methyl-2,3,7,11,14,23-hexaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (3-nitrophenyl)hydrazine hydrochloride (2.6 g, 13.7 mmol) in ethanol (50 mL) was added DIEA (1.77 g, 13.7 mmol) and tert-butyl(2S)-3-cyano-2-methyl-4-oxo-1-piperidinecarboxylate (3.27 g, 13.7 mmol) and the mixture was stirred at 80° C. for 16 hours. The reaction mixture was concentrated, and the crude product was purified by column chromatography (0-25% EtOAc in PE) to give tert-butyl (S)-3-amino-4-methyl-2-(3-nitrophenyl)-2,4,6,7-tetrahydro-5H-pyrazolo[4,3-c]pyridine-5-carboxylate (3 g, 7.23 mmol) as a yellow solid. m/z (ESI-pos) M+1=374.1.

Step B: To a solution of tert-butyl (S)-3-amino-4-methyl-2-(3-nitrophenyl)-2,4,6,7-tetrahydro-5H-pyrazolo[4,3-c]pyridine-5-carboxylate (3 g, 7.23 mmol) in THF (39.1 mL) was added DIEA (2.8 g, 21.7 mmol) and benzenyl chloroformate (2.26 g, 14.5 mmol). The mixture was stirred at ambient temperature for 1 hour and then directly concentrated under vacuum to get crude tert-butyl (S)-4-methyl-2-(3-nitrophenyl)-3-((phenoxycarbonyl)amino)-2,4,6,7-tetrahydro-5H-pyrazolo[4,3-c]pyridine-5-carboxylate (4.4 g, 7.13 mmol) as a light yellow solid, which was used directly in next step without further purification. m/z (ESI-pos) M+1=494.3.

Step C: To a solution of tert-butyl (S)-4-methyl-2-(3-nitrophenyl)-3-((phenoxycarbonyl)amino)-2,4,6,7-tetrahydro-5H-pyrazolo[4,3-c]pyridine-5-carboxylate (4.4 g, 7.13 mmol) in pyridine (20 mL) was added 2,2-dimethoxyethylamine (2.25 g, 21.4 mmol). The mixture was stirred at ambient temperature for 3 hours and then directly concentrated to get the crude product, which was purified by column chromatography (0-66% EtOAc in PE) to give tert-butyl (S)-3-(3-(2,2-dimethoxyethyl)ureido)-4-methyl-2-(3-nitrophenyl)-2,4,6,7-tetrahydro-5H-pyrazolo[4,3-c]pyridine-5-carboxylate (3.5 g, 6.24 mmol) as a white solid. m/z (ESI-pos) M+1=505.2.

Step D: To a solution of tert-butyl (S)-3-(3-(2,2-dimethoxyethyl)ureido)-4-methyl-2-(3-nitrophenyl)-2,4,6,7-tetrahydro-5H-pyrazolo[4,3-c]pyridine-5-carboxylate (3.5 g, 6.24 mmol) in THF (60 mL) was added methanesulfonic acid (1.8 g, 18.7 mmol). The mixture was stirred at 60° C. for 2 h and then cooled to ambient temperature, then adjusted the pH to about 9 with added aqueous K₃PO₄. Then Boc20 (6.8 g, 31.2 mmol) was added and the mixture was stirred for 16 hours. The pH was adjusted to about 7 with aq. Citric acid, and the mixture was extracted with EtOAc (2×). The combined organic layers were dried over anhydrous Na₂SO₄, filtered and concentrated. The crude product was purified by column chromatography (0-50% EtOAc in PE) to give tert-butyl (S)-4-methyl-2-(3-nitrophenyl)-3-(2-oxo-2,3-dihydro-1H-imidazol-1-yl)-2,4,6,7-tetrahydro-5H-pyrazolo[4,3-c]pyridine-5-carboxylate (2.65 g, 4.41 mmol) as a white solid. m/z (ESI-pos) M+1=541.2.

Step E: To a solution of tert-butyl (S)-4-methyl-2-(3-nitrophenyl)-3-(2-oxo-2,3-dihydro-1H-imidazol-1-yl)-2,4,6,7-tetrahydro-5H-pyrazolo[4,3-c]pyridine-5-carboxylate (2.65 g, 4.41 mmol) in methanol (50 mL) was added Pd/C (470 mg, 4.41 mmol). The mixture was stirred under H₂ atmosphere at ambient temperature for 2 hours, then filtered through a pad of celite. The filtrate was concentrated under reduced pressure to give tert-butyl (S)-2-(3-aminophenyl)-3-(3-(tert-butoxycarbonyl)-2-oxo-2,3-dihydro-1H-imidazol-1-yl)-4-methyl-2,4,6,7-tetrahydro-5H-pyrazolo[4,3-c]pyridine-5-carboxylate (2.3 g, 4.05 mmol) as a white solid, which was used in the next step without further purification. m/z (ESI-pos) M+1=511.2

Step F: To a solution of tert-butyl (S)-2-(3-aminophenyl)-3-(3-(tert-butoxycarbonyl)-2-oxo-2,3-dihydro-1H-imidazol-1-yl)-4-methyl-2,4,6,7-tetrahydro-5H-pyrazolo[4,3-c]pyridine-5-carboxylate (2.3 g, 4.05 mmol) in MeOH (50 mL) was added (o-bromophenyl) acetaldehyde (897 mg, 4.05 mmol). The mixture was stirred at ambient temperature for 1 hour. Then NaBH₃CN (764 mg, 12.2 mmol) was added and the mixture was stirred for 24 hours. The reaction solution was quenched with water, extracted with EtOAc (3×), the combined organic layers were dried over anhydrous Na₂SO₄, filtered and the filtrate was concentrated under vacuum. The crude product was purified by column chromatography (0-50% EtOAc in PE) to give tert-butyl (S)-2-(3-((2-bromophenethyl)amino)phenyl)-3-(3-(tert-butoxycarbonyl)-2-oxo-2,3-dihydro-1H-imidazol-1-yl)-4-methyl-2,4,6,7-tetrahydro-5H-pyrazolo[4,3-c]pyridine-5-carboxylate (2.18 g, 2.83 mmol) as a white solid. m/z (ESI-pos) M+1=693.2, 695.2.

Step G: To a solution of tert-butyl (S)-2-(3-((2-bromophenethyl)amino)phenyl)-3-(3-(tert-butoxycarbonyl)-2-oxo-2,3-dihydro-1H-imidazol-1-yl)-4-methyl-2,4,6,7-tetrahydro-5H-pyrazolo[4,3-c]pyridine-5-carboxylate (1.4 g, 1.82 mmol) in methanol (50 mL) was added formaldehyde (545 mg, 18.2 mmol) and acetic acid (218 mg, 3.63 mmol). The mixture was stirred at ambient temperature for 30 mins, then NaBH₃CN (571 mg, 9.08 mmol) was added into and the mixture was stirred for 16 hours. The reaction was quenched with water, then extracted with EtOAc (3×) and the combined organic layers were dried over anhydrous Na₂SO₄, filtered and the filtrate was concentrated under vacuum to give crude tert-butyl (S)-2-(3-((2-bromophenethyl)(methyl)amino)phenyl)-3-(3-(tert-butoxycarbonyl)-2-oxo-2,3-dihydro-1H-imidazol-1-yl)-4-methyl-2,4,6,7-tetrahydro-5H-pyrazolo[4,3-c]pyridine-5-carboxylate (0.8 g, 1.02 mmol) as a white solid. m/z (ESI-pos) M+1=707.2, 709.2.

Step H: To a solution of tert-butyl (S)-2-(3-((2-bromophenethyl)(methyl)amino)phenyl)-3-(3-(tert-butoxycarbonyl)-2-oxo-2,3-dihydro-1H-imidazol-1-yl)-4-methyl-2,4,6,7-tetrahydro-5H-pyrazolo[4,3-c]pyridine-5-carboxylate (0.8 g, 1.02 mmol) in methanol (20 mL) was added K₂CO₃ (703 mg, 5.09 mmol). The mixture was stirred at ambient temperature for 1 hour and then diluted with water. The mixture was extracted with EtOAc (3×), the combined organic layers were dried over anhydrous Na₂SO₄, filtered and the filtrate was concentrated under vacuum. The crude product was purified by column chromatography (0-66% EtOAc in PE) to give tert-butyl (S)-2-(3-((2-bromophenethyl)(methyl)amino)phenyl)-4-methyl-3-(2-oxo-2,3-dihydro-1H-imidazol-1-yl)-2,4,6,7-tetrahydro-5H-pyrazolo[4,3-c]pyridine-5-carboxylate (640 mg, 948 μmol) as a white solid. m/z (ESI-pos) M+1=607.1, 609.1.

Step I: To a solution of tert-butyl (S)-2-(3-((2-bromophenethyl)(methyl)amino)phenyl)-4-methyl-3-(2-oxo-2,3-dihydro-1H-imidazol-1-yl)-2,4,6,7-tetrahydro-5H-pyrazolo[4,3-c]pyridine-5-carboxylate (640 mg, 948 μmol) in DMSO (19 mL) was added CuI (135 mg, 711 μmol), 1,8-diphenyl-4,5-diazaphenanthrene (315 mg, 948 μmol) and K₂CO₃ (328 mg, 2.37 mmol). The mixture was sealed and stirred at 120° C. in an oil bath for 48 hrs. The reaction mixture was cooled to ambient temperature, diluted with water and extracted with EtOAc (3×). The combined organic layers were washed with brine (3×), dried over anhydrous Na₂SO₄, filtered and the filtrate was concentrated under vacuum. The crude product was purified by column chromatography (0-50% EtOAc in PE) to give tert-butyl (S)-8-methyl-23-methyl-29-oxo-2,3,7,11,14,23-hexaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (0.1 g, 171 μmol) as a yellow solid. The isomers were separated by chiral supercritical fluid chromatography [Column: Daicel CHIRALCEL IG, 250 mm*30 mm I.D., 10 μm; Mobile Phase: CO2/MeOH [0.2% NH₃ (7M Solution in MeOH)]=40/60; Flow rate: 80 g/min; Temp: 35° C.; Wavelength: 214 nm] to afford (Isomer 1, 13 mg, 23.5 μmol) and (Isomer 2, 50 mg, 90.2 μmol) as white solids (The absolute configurations were unknown and assigned arbitrarily). Isomer 1 and Isomer 2: m/z (ESI-pos) M+1=527.2.

Step J: To a solution of tert-butyl (S)-8-methyl-23-methyl-29-oxo-2,3,7,11,14,23-hexaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (Isomer 1, 13 mg, 23.5 μmol) in DCM (1 mL, 15.6 mmol) was added HCl/1,4-dioxane (1 mL, 23.5 μmol). The mixture was stirred at ambient temperature for 1 hour and then concentrated to give crude(S)-8-methyl-23-methyl-2,3,7,11,14,23-hexaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (Isomer 1, 10 mg, 22.3 μmol) as a white solid. m/z (ESI-pos) M+1=427.2.

Step K: To a solution of(S)-8-methyl-23-methyl-2,3,7,11,14,23-hexaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (Isomer 1, 10 mg, 22.3 μmol) in DMF (2.5 mL) was added 5-[(S)-2,2-dimethyltetrahydro-4H-pyran-4-yl]-1-[1-(5-oxo-4,5-dihydro-1,2,4-oxadiazol-3-yl)cyclopropyl]-2-indolecarboxylic acid (8.85 mg, 22.3 μmol) and DIEA (11.5 mg, 89.1 μmol). The mixture was stirred for 10 mins and then HATU (11 mg, 29 μmol) was added, and the mixture was stirred at ambient temperature for 16 hours. The reaction was quenched with water and extracted with EtOAc (2×). The combined EtOAc layers were washed with brine, dried over Na₂SO₄, filtered and concentrated in vacuo. The residue was purified by column chromatography (0-5% MeOH in DCM) to give the crude product, which was further purified by prep-HPLC [Instrument: Waters Prep-LC with UV Detector; Column: Waters Sunfire C18 5 um 50*150 mm; Gradient: eluted with 55-98% ACN in H₂O (0.1% FA) in 15 min; Flow rate: 15 mL/min] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-23-methyl-2,3,7,11,14,23-hexaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 1, 6.23 mg, 7.46 μmol) as a white solid. m/z (ESI-pos) M+1=820.3; ¹H NMR (400 MHz, DMSO) δ 11.61 (s, 1H), 7.52-7.31 (m, 7H), 7.24-7.22 (m, 1H), 6.91-6.81 (m, 3H), 6.68-6.60 (m, 3H), 5.60 (s, 1H), 4.39 (s, 1H), 3.73-3.71 (m, 2H), 3.60-3.53 (m, 2H), 3.24-3.16 (m, 2H), 3.02-3.01 (m, 2H), 2.99 (s, 3H), 2.87-2.82 (m, 1H), 2.76-2.68 (m, 1H), 2.40-2.34 (m, 1H), 1.73-1.68 (m, 3H), 1.60-1.50 (m, 5H), 1.28-1.25 (m, 10H).

Step L: Steps J and K were repeated using Isomer 2 in place of Isomer 1 to provide (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-23-methyl-2,3,7,11,14,23-hexaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (Isomer 2, 32.6 mg, 38.4 μmol) as a white solid. m/z (ESI-pos) M+1=820.3; ¹ HNMR (400 MHz, DMSO) δ 11.59 (s, 1H), 7.52-7.39 (m, 6H), 7.32 (t, J=8.4 Hz, 1H), 7.24 (d, J=8.4 Hz, 1H), 6.92-6.85 (m, 3H), 6.65-6.63 (m, 3H), 5.58 (s, 1H), 4.42 (s, 1H), 3.73-3.71 (m, 2H), 3.63-3.57 (m, 2H), 3.24-3.16 (m, 2H), 3.03-3.01 (m, 2H), 2.98 (s, 3H), 2.91-2.87 (m, 1H), 2.75-2.68 (m, 1H), 2.37-2.31 (m, 1H), 1.74-1.68 (m, 3H), 1.63-1.50 (m, 5H), 1.28-1.25 (m, 4H), 1.19 (s, 6H).

### Example 50—Synthesis of Compound 50

### Synthetic Scheme

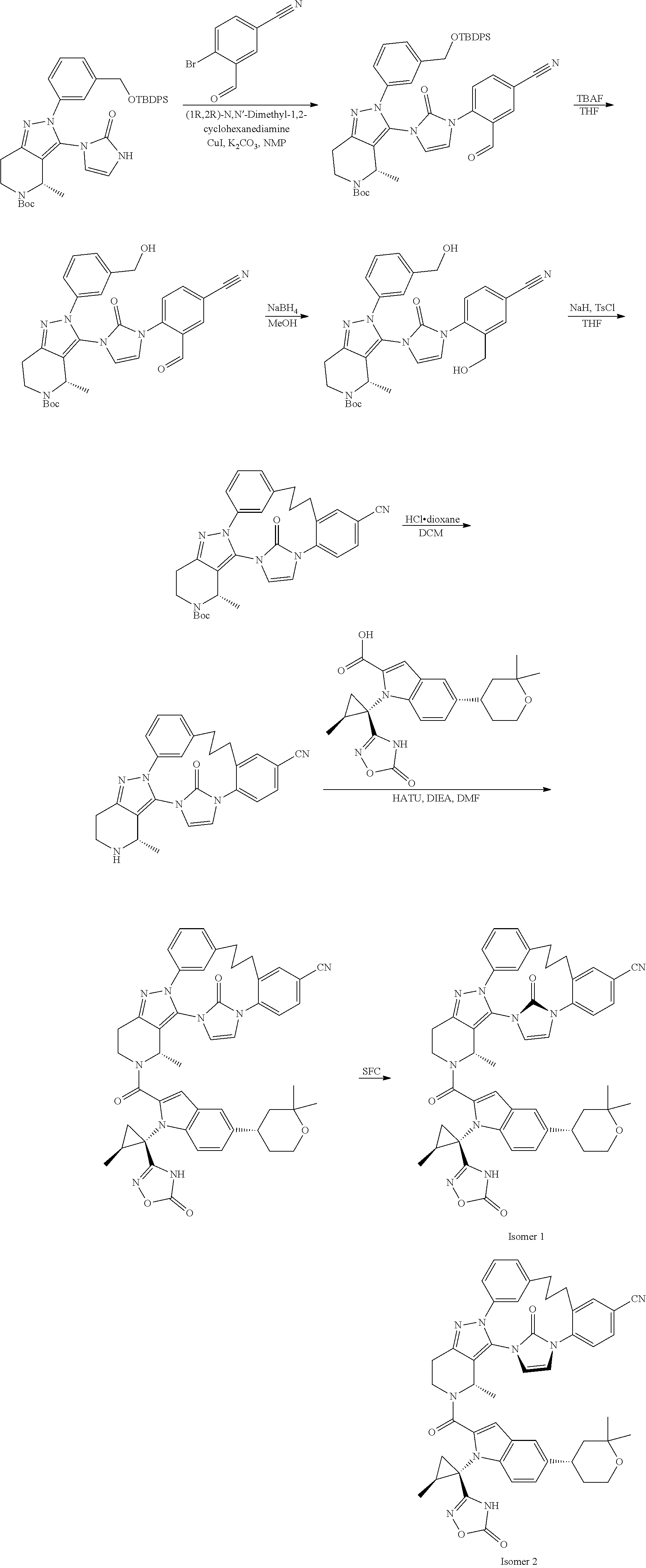

### Procedure for Synthesis of Compound 50

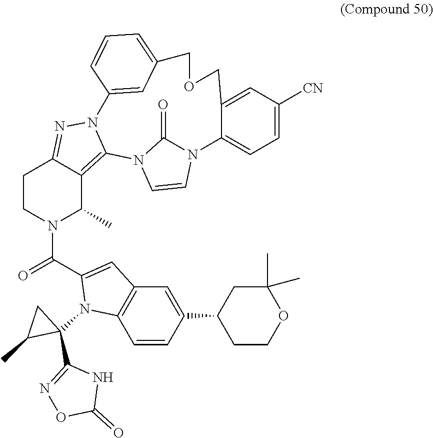

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-18-carbonitrile

Step A: To a solution of tert-butyl (S)-2-(3-(((tert-butyldiphenylsilyl)oxy)methyl)phenyl)-4-methyl-3-(2-oxo-2,3-dihydro-1H-imidazol-1-yl)-2,4,6,7-tetrahydro-5H-pyrazolo[4,3-c]pyridine-5-carboxylate (1.4 g, 1.9 mmol) and 4-bromo-3-formylbenzonitrile (1.20 g, 5.69 mmol) in NMP (50 mL) was added copper iodide (361 mg, 1.9 mmol), rel-(1R,2R)-1,2-bis(methylamino)cyclohexane (540 mg 3.8 mmol) and dipotassium carbonate (787 mg, 5.69 mmol). The mixture was stirred at 130° C. for 4 hours. The reaction mixture was cooled to ambient temperature and diluted with H₂O, then extracted with EtOAc. The combined organic layers were washed with aq. LiCl (2×) and concentrated under reduced pressure. The crude product was purified by column chromatography (0-33% EtOAc in PE) to give tert-butyl (S)-2-(3-(((tert-butyldiphenylsilyl)oxy)methyl)phenyl)-3-(3-(4-cyano-2-formylphenyl)-2-oxo-2,3-dihydro-1H-imidazol-1-yl)-4-methyl-2,4,6,7-tetrahydro-5H-pyrazolo[4,3-c]pyridine-5-carboxylate (1 g, 1.01 mmol) as a yellow solid. m/z (ESI-pos) M+1=793.3.

Step B: To a solution of tert-butyl (S)-2-(3-(((tert-butyldiphenylsilyl)oxy)methyl)phenyl)-3-(3-(4-cyano-2-formylphenyl)-2-oxo-2,3-dihydro-1H-imidazol-1-yl)-4-methyl-2,4,6,7 tetrahydro-5H-pyrazolo[4,3-c]pyridine-5-carboxylate (0.9 g, 908 μmol) in tetrahydrofuran (40 mL) at 0° C. was added tetrabutylammonium fluoride (1.0 M in THF, 1.36 mL, 1.36 mmol). The mixture allowed to warm to ambient temperature where it stirred for 1 hour. The reaction was quenched with H₂O and extracted with EtOAc (2×). The combined organic layers were washed with brine and concentrated under vacuum. The crude product was purified by column chromatography (0-70% EtOAc in PE) to give tert-butyl (S)-3-[3-(4-cyano-2-formylphenyl)-2-oxo-2,3-dihydro-1H-imidazol-1-yl]-2-[m-(hydroxymethyl)phenyl]-4-methyl-1,2,5-triaza-2,4,6,7-tetrahydro-5H-indene-5-carboxylate (350 mg, 568 μmol) as a yellow solid. m/z (ESI-pos) M+1=555.2.

Step C: To a solution of tert-butyl (S)-3-[3-(4-cyano-2-formylphenyl)-2-oxo-2,3-dihydro-1H-imidazol-1-yl]-2-[m-(hydroxymethyl)phenyl]-4-methyl-1,2,5-triaza-2,4,6,7-tetrahydro-5H-indene-5-carboxylate (350 mg, 568 μmol) in methanol (10 mL) at 0° C. was added NaBH₄ (107 mg, 2.84 mmol) and the mixture was stirred at 20° C. for 16 hours. The reaction was quenched with H₂O and then extracted with EtOAc (2×). The combined organic layers were washed with brine, dried, and concentrated under reduced pressure. The crude product was purified by column chromatography (0-80% EtOAc in PE) to give tert-butyl (S)-3-{3-[4-cyano-2-(hydroxy methyl)phenyl]-2-oxo-2,3-dihydro-1H-imidazol-1-yl}-2-[m-(hydroxy methyl)phenyl]-4-methyl-1,2,5-triaza-2,4,6,7-tetrahydro-5H-indene-5-carboxylate (260 mg, 234 μmol) as a yellow solid. m/z (ESI-pos) M+1=557.2.

Step D: To a solution of tert-butyl (S)-3-{3-[4-cyano-2-(hydroxymethyl)phenyl]-2-oxo-2,3-dihydro-1H-imidazol-1-yl}-2-[m-(hydroxymethyl)phenyl]-4-methyl-1,2,5-triaza-2,4,6,7-tetrahydro-5H-indene-5-carboxylate (260 mg, 234 μmol) in THF (16 mL) at 0° C. was added sodium hydride (60% w/w, 28 mg, 701 μmol) and the mixture was stirred at 0° C. for 30 mins. Then p-(chlorosulfonyl) toluene (31.2 mg, 163 μmol) in THF (4 mL) was added dropwise. The mixture was stirred at 0° C. for 1 hour. The reaction was quenched with water and extracted with EtOAc (2×). The combined organic layers were washed with brine, dried over Na₂SO₄ and concentrated. The crude product was purified by column chromatography (0-50% EtOAc in PE) to give tert-butyl (S)-18-cyano-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (110 mg, 184 μmol) as a yellow solid. m/z (ESI-pos) M+1=539.2.

Step E: To a solution of tert-butyl (S)-18-cyano-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (110 mg, 184 μmol) in DCM (5 mL) was added HCl/1,4-dioxane (4M, 5 mL). The reaction mixture was stirred at ambient temperature for 2 hours and then concentrated in vacuo to get crude(S)-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-18-carbonitrile hydrogen chloride (89 mg, 183 μmol) as a white solid. m/z (ESI-pos) M+1=439.1

Step F: To a solution of(S)-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-18-carbonitrile hydrogen chloride (89 mg, 183 μmol) in DMF (5 mL) was added 5-[(S)-2,2-dimethyltetrahydro-4H-pyran-4-yl]-1-[(2S)-2-methyl-1-(5-oxo-4,5-dihydro-1,2,4-oxadiazol-3-yl)cyclo propyl]-2-indolecarboxylic acid (75.2 mg, 183 μmol) and DIEA (94.4 mg, 731 μmol). The reaction mixture was stirred at ambient temperature for 10 mins and then HATU (90.3 mg, 237 μmol) was added. The mixture was stirred for 16 hours. H₂O was added, and the mixture was extracted with EtOAc (2×). The combined EtOAc layers were washed with brine and dried over Na₂SO₄, filtered and concentrated in vacuo. The crude product was purified by column chromatography (0-5% MeOH in DCM) to give 150 mg crude product, which was purified by prep-HPLC [Instrument: GILSON Prep LC with UV detector; Column: XBridge Prep C18 19*150 mm 5 um; Gradient: eluted with 60-90% ACN in H2O (0.1% FA) in 10 min; Flow rate: 25 ml/min] to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-18-carbonitrile (100 mg, 114 μmol) as a white solid. m/z (ESI-pos) M+1=832.2.

Step G: A solution of(S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-18-carbonitrile (100 mg, 114 μmol) in MeOH (10 mL) was separated by chiral supercritical fluid chromatography [separation condition: Daicel CHIRALCEL IH, 250 mm*30 mm I.D., 10 μm; Mobile Phase: CO2/MeOH [0.2% NH₃ (7M Solution in MeOH)]=50/50 at 120 g/min; Temp: 35° C.; Wavelength: 214 nm] to afford Isomer 1 (48.8 mg) and Isomer 2 (30.7 mg) as white solids (The absolute configuration was unknown, the configuration of two atropisomers were assigned arbitrarily). Isomer 1: m/z (ESI-pos) M+1=832.3; ¹HNMR (400 MHz, DMSO) δ 11.54 (s, 1H), 8.05 (d, J=1.6 Hz, 1H), 7.95 (d, J=8.0 Hz, 1H), 7.67 (d, J=8.0 Hz, 1H), 7.58 (d, J=8.4 Hz, 1H), 7.51-7.47 (m, 2H), 7.40 (d, J=8.4 Hz, 1H), 7.29-7.22 (m, 2H), 7.06 (s, 1H), 6.83-6.73 (m, 3H), 5.63 (s, 1H), 4.65-4.58 (m, 2H), 4.43 (s, 1H), 4.27 (d, J=9.2 Hz, 1H), 4.13 (d, J=9.6 Hz, 1H), 3.73-3.71 (m, 2H), 3.57 (s, 1H), 3.01-2.99 (m, 2H), 2.92-2.88 (m, 1H), 1.81-1.68 (m, 4H), 1.65-1.50 (m, 5H), 1.28-1.25 (m, 4H), 1.19 (s, 6H). Isomer 2: m/z (ESI-pos) M+1=832.3; ¹HNMR (400 MHz, DMSO) δ 11.37 (s, 1H), 8.08 (s, 1H), 7.94 (d, J=6.8 Hz, 1H), 7.74 (s, 1H), 7.57 (d, J=8.4 Hz, 1H), 7.52-7.49 (m, 2H), 7.44-7.39 (m, 1H), 7.29 (d, J=7.6 Hz, 1H), 7.23 (d, J=8.8 Hz, 1H), 7.07 (s, 1H), 6.86-6.81 (m, 2H), 6.63 (d, J=3.2 Hz, 1H), 5.64 (s, 1H), 4.70-4.57 (m, 2H), 4.42 (s, 1H), 4.26-4.17 (m, 2H), 3.73-3.71 (m, 2H), 3.57 (s, 1H), 3.02-2.98 (m, 2H), 2.90-2.86 (m, 1H), 1.71-1.69 (m, 4H), 1.61-1.49 (m, 5H), 1.28-1.25 (m, 6H), 1.19 (s, 4H).

### Example 51—Synthesis of Compound 51

### Procedure for Synthesis of Compound 51

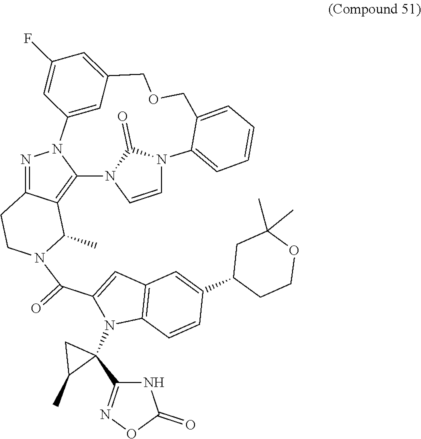

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 51 was synthesized following the procedure of Example 32, with (2-fluoro-5-iodo-3-tolyl)methanol and o-bromo(bromomethyl)benzene being replaced with (o-bromophenyl) methanol and 1-(bromomethyl)-3-fluoro-5-iodobenzene in step C. m/z (ESI-pos) M+1=825.5; ¹H NMR (400 MHz, CHLOROFORM-D) δ 11.31 (s, 1H), 7.58 (dd, J=8.6, 4.7 Hz, 1H), 7.56-7.30 (m, 6H), 7.28-7.25 (obs m, 1H), 7.00-6.87 (m, 2H), 6.80-6.68 (m, 1H), 6.55-5.91 (m, 2H), 5.85-5.24 (m, 1H), 4.75-4.53 (m, 2H), 4.48 (dd, J=13.7, 5.1 Hz, 1H), 4.34 (dd, J=35.3, 9.3 Hz, 1H), 4.07 (d, J=9.4 Hz, 1H), 3.92-3.78 (m, 2H), 3.69-3.33 (m, 1H), 3.20-2.98 (m, 3H), 1.88 (dt, J=23.0, 6.0 Hz, 1H), 1.81-1.62 (m, 6H), 1.61-1.37 (obs m, 3H), 1.30 (d, J=24.0 Hz, 6H), 1.16 (dd, J=28.0, 6.0 Hz, 3H).

### Example 52—Synthesis of Compound 52

### Synthetic Scheme

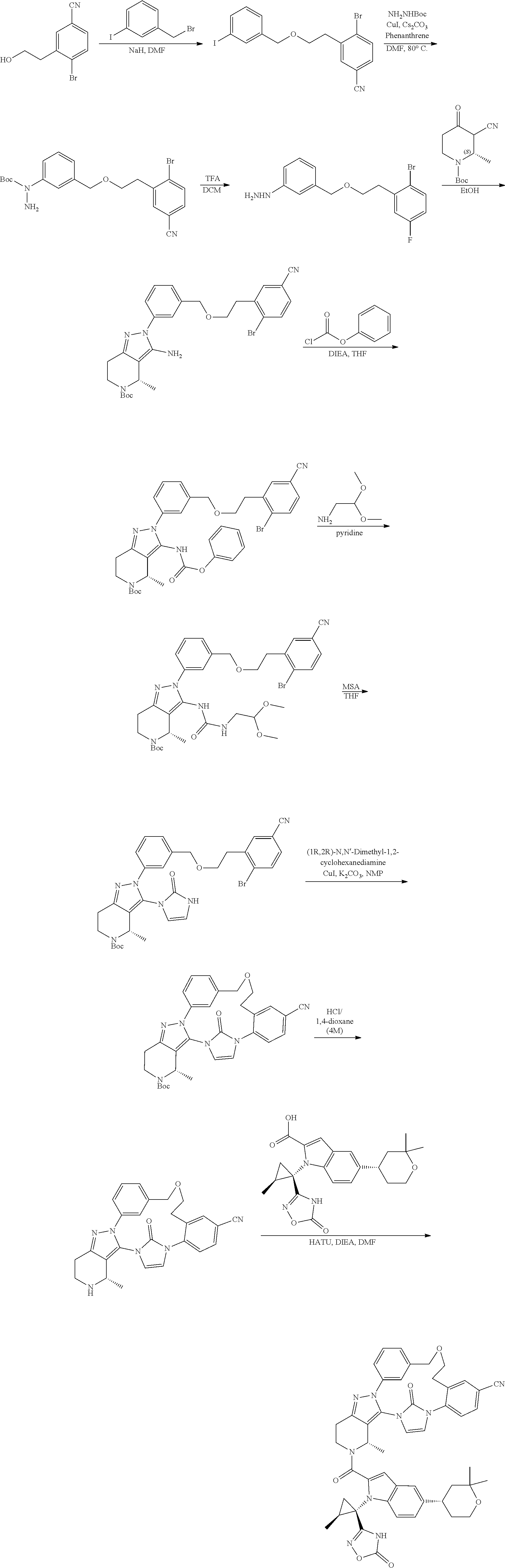

### Procedure for Synthesis of Compound 52

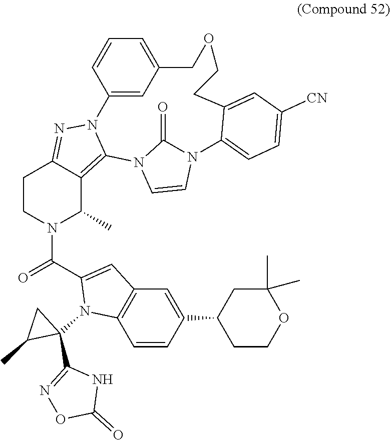

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-18-carbonitrile

Step A: To the solution of 4-bromo-3-(2-hydroxyethyl)benzonitrile (0.4 g, 1.77 mmol) in dimethylformamide (11.8 mL) was added sodium hydride 60% w/w (398 mg, 9.95 mmol) at 0° C. under N₂. The solution was stirred at 0° C. for 5 min. m-(bromomethyl) iodobenzene (788 mg, 2.65 mmol) was then added in one portion at 0° C. and the reaction was warmed to ambient temperature and stirred for 2 hours. The reaction mixture was quenched with addition of water and saturated aqueous NH₄Cl. The reaction mixture was extracted with EtOAc (3×). The combined organic layers were washed with brine (2×) then dried over Na₂SO₄, filtered and concentrated in vacuo to afford the crude product which was then purified via column chromatography (0-20% EtOAc in Heptane) to give 4-bromo-3-{2-[(m-iodophenyl)methoxy]ethyl}benzonitrile (616 mg, 1.39 mmol). ¹H NMR (400 MHz, CHLOROFORM-D) δ 7.69-7.54 (m, 4H), 7.36 (dd, J=8.2, 2.1 Hz, 1H), 7.23-7.18 (m, 1H), 7.06 (td, J=7.5, 1.1 Hz, 1H), 4.44 (s, 2H), 3.69 (t, J=6.5 Hz, 2H), 3.07 (t, J=6.5 Hz, 2H).

Step B: A mixture of 4-bromo-3-{2-[(m-iodophenyl)methoxy]ethyl}benzonitrile (391 mg, 884 μmol), dicesium carbonate (403 mg, 1.24 mmol), copper iodide (8.42 mg, 44.2 μmol), 4,5-diazaphenanthrene (31.9 mg, 177 μmol), and tert-butyl carbazate (152 mg, 1.15 mmol) in dimethylformamide (884 μL, 11.4 mmol) was sparged with Ar for 5 min and heated to 80° C. for 5 h then cooled to ambient temperature. The mixture was taken up in EtOAc and washed with saturated aqueous NH₄Cl (2×) followed by brine. The organics were dried over Na₂SO₄, filtered, and concentrated. The crude product was purified by silica gel chromatography (5-40% EtOAc/heptane) to give 4-bromo-3-(2-{[m-(tert-butyl-2-carboxyhydrazino)phenyl]methoxy}ethyl)benzonitrile (255 mg, 571 μmol). m/z (ESI-pos) M+1=390.1, 392.1 (−tBu)

Step C: To a solution of 4-bromo-3-(2-{[m-(tert-butyl-2-carboxyhydrazino)phenyl]methoxy}ethyl)benzonitrile (255 mg, 571 in μmol) dichloromethane (1.9 mL) was added trifluoroacetic acid (437 μL, 5.71 mmol) at ambient temperature. The reaction was stirred at ambient temperature for 3 h. The reaction diluted with CH₂Cl₂, cooled to 0° C. and quenched with saturated aqueous Na₂CO₃ (2 mL). The mixture was diluted further with water and CH₂Cl₂ and the layers were mixed and separated, and the organic layer was washed with water followed by brine. The combined aqueous layers were then back extracted with CH₂Cl₂ (2×), and the combined organics were washed with brine (1×) and then dried over Na₂SO₄, filtered, and concentrated in vacuo to provide 4-bromo-3-{2-[(m-hydrazinophenyl)methoxy]ethyl}benzonitrile which was used immediately, without purification in the following step. m/z (ESI-pos) M+1=346.1, 348.1

Step D: To a solution of 4-bromo-3-{2-[(m-hydrazinophenyl)methoxy]ethyl}benzonitrile (198 mg, 572 μmol) in ethanol (2.29 mL) was added tert-butyl(2S)-3-cyano-2-methyl-4-oxo-1-piperidinecarboxylate (164 mg, 686 μmol) followed by N-ethyldiisopropylamine (149 μL, 858 μmol). The mixture was then warmed to 80° C. where it stirred for 42 hours. The reaction mixture was cooled down to room temperature and concentrated. The residue was taken up in EtOAc, washed with saturated aqueous NH₄Cl, followed by water. The organic layer was then washed with brine solution, dried over anhydrous Na₂SO₄, filtered and the filtrate was evaporated under reduced pressure to give the crude compound which was purified by column chromatography on silica gel (25-55% EtOAc/Heptane) to afford tert-butyl (S)-3-amino-2-(m-{[2-(2-bromo-5-cyanophenyl)ethoxy]methyl}phenyl)-4-methyl-1,2,5-triaza-2,4,6,7-tetrahydro-5H-indene-5-carboxylate (141 mg, 249 μmol). m/z (ESI-pos) M+1=566.3, 568.3

Steps E through J were followed as outlined in Example 23 to afford(S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-18-carbonitrile (22 mg, 26 μmol). m/z (ESI-pos) M+1=846.6; ¹H NMR (400 MHz, CHLOROFORM-D) δ 11.25 (s, 1H), 7.68-7.40 (m, 7H), 7.32-7.27 (m, 1H), 7.22-7.03 (m, 2H), 6.76-6.66 (m, 1H), 6.50-6.43 (m, 1H), 6.35-5.98 (m, 1H), 5.89-5.23 (m, 1H), 4.73-4.43 (m, 2H), 3.91-3.56 (m, 2H), 3.44-2.94 (m, 7H), 1.92-1.70 (m, 4H), 1.69-1.56 (m, 5H), 1.54-1.48 (m, 3H), 1.34-1.25 (m, 6H), 1.20-1.00 (m, 3H).

### Example 53—Synthesis of Compound 53

### Synthetic Scheme

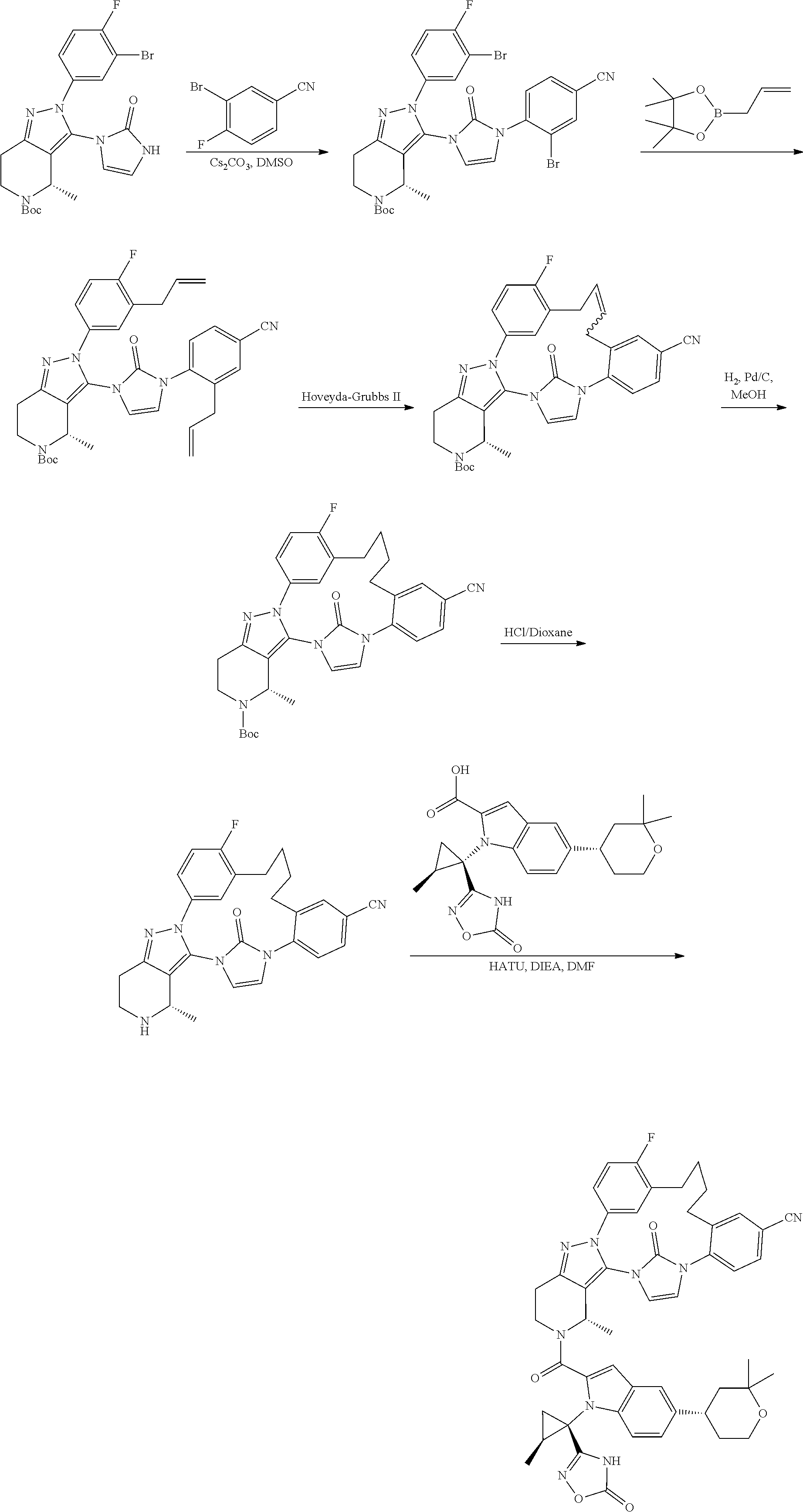

### Procedure for Synthesis of Compound 53

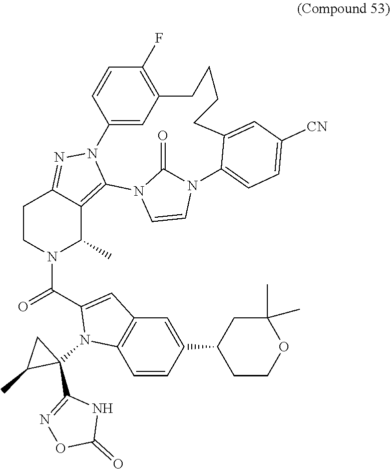

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-18-carbonitrile

Step A: To a solution of tert-butyl (S)-2-(3-bromo-4-fluorophenyl)-4-methyl-3-(2-oxo-2,3-dihydro-1H-imidazol-1-yl)-1,2,5-triaza-2,4,6,7-tetrahydro-5H-indene-5-carboxylate (74 mg, 150 μmol) in DMSO (0.2 mL) was added dicesium carbonate (88.1 mg, 271 μmol). The mixture was stirred at room temperature for 5 min, followed by the addition of 3-bromo-4-fluorobenzonitrile (63.1 mg, 316 μmol). The reaction mixture was heated at 40° C. for 60 min, cooled to room temperature, and quenched by the addition of H₂O and saturated aqueous NH₄Cl. The mixture was extracted with EtOAc (3×). The combined organic layers were washed with brine then dried over Na₂SO₄, filtered and concentrated in vacuo. The residue was purified by flash column chromatography (20-50% EtOAc in Heptane), to give tert-butyl (S)-3-[3-(2-bromo-4-cyanophenyl)-2-oxo-2,3-dihydro-1H-imidazol-1-yl]-2-(3-bromo-4-fluorophenyl)-4-methyl-1,2,5-triaza-2,4,6,7-tetrahydro-5H-indene-5-carboxylate (82 mg, 122 μmol). m/z (ESI-pos) M+1=673.2

Step B: tert-butyl (S)-3-[3-(2-bromo-4-cyanophenyl)-2-oxo-2,3-dihydro-1H-imidazol-1-yl]-2-(3-bromo-4-fluorophenyl)-4-methyl-1,2,5-triaza-2,4,6,7-tetrahydro-5H-indene-5-carboxylate (80 mg, 119 μmol), bis[2-(diphenylphosphino)-2,4-cyclopentadien-1-ide]-dichloro-palladamethane-dichloromethane-iron (1/1/1/1) (9.72 mg, 11.9 μmol), and dipotassium carbonate (49.3 mg, 357 μmol) were dissolved in 1,4-dioxane (793 μL) and water (119 μL). 2-allyl-4,4,5,5-tetramethyl-1,3,2-dioxaborolane (134 μL, 714 μmol) was added and the mixture was degassed with Ar for 5 min then heated to 80° C. where it stirred for 3 hours. Then additional 2-allyl-4,4,5,5-tetramethyl-1,3,2-dioxaborolane (134 μL, 714 μmol) was added and the mixture was stirred for another 6 hours. After cooling to ambient temperature, the reaction was diluted with water and extracted with EtOAc (3×). The combined organic layers were washed with brine, then dried over Na₂SO₄, filtered and concentrated. The crude material was purified by column chromatography (20-50% EtOAc/heptane) to give tert-butyl (S)-3-[3-(2-allyl-4-cyanophenyl)-2-oxo-2,3-dihydro-1H-imidazol-1-yl]-2-(3-allyl-4-fluorophenyl)-4-methyl-1,2,5-triaza-2,4,6,7-tetrahydro-5H-indene-5-carboxylate (50 mg, 84.1 μmol). m/z (ESI-pos) M+1=595.4.

Step C: tert-butyl (S)-3-[3-(2-allyl-4-cyanophenyl)-2-oxo-2,3-dihydro-1H-imidazol-1-yl]-2-(3-allyl-4-fluorophenyl)-4-methyl-1,2,5-triaza-2,4,6,7-tetrahydro-5H-indene-5-carboxylate (57 mg, 95.8 μmol) was dissolved in dichloromethane (31.9 mL). The mixture was warmed to 35° C. and then ruthenium o-isopropoxymethanediidyltoluene 1,3-bis(mesityl)-2,2-imidazolidinediide dichloride (15 mg, 24 μmol) was added and the mixture was stirred for 2 hours. The reaction was cooled to ambient temperature, concentrated and the crude material was purified by silica gel chromatography (10-30% EtOAc/heptane) to give tert-butyl (S)-18-cyano-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (40 mg, 70.6 μmol). m/z (ESI-pos) M+1=567.4

Step D: To a solution of tert-butyl (S)-18-cyano-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (42 mg, 74.1 μmol) in ethyl acetate (1.48 mL, 15 mmol) was added palladium 5% w/w (15.8 mg, 7.41 μmol) and the mixture was then purged with H₂ gas and the mixture was allowed to stir for 3 hours under a H₂ atmosphere with balloon pressure at ambient temperature. The mixture was then purged with Nitrogen gas and the mixture was filtered through a nylon filter. The filter was then washed with EtOAc and the filtrate was concentrated and the crude residue was purified by column chromatography on silica gel (10-40% EtOAc/Heptane) to afford the desired product as a white solid. m/z (ESI-pos) M+1=569.4

Step E: tert-butyl (S)-18-cyano-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (33 mg, 58 μmol) was treated with 4N HCl in Dioxane (0.725 mL, 2.9 mmol) and the mixture was stirred at ambient temperature for 1 hour. The mixture was then added dropwise to a stirring solution of saturated aqueous NaHCO₃. The mixture was then extracted with 10% IPA/CHCl₃ (3×) and the combined extracts were dried over Na₂SO₄, filtered and concentrated to provide (S)-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-18-carbonitrile (27 mg, 57.6 μmol). m/z (ESI-pos) M+1=469.4

Step F: To a solution of(S)-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-18-carbonitrile (27 mg, 57.6 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (30.8 mg, 74.9 μmol) in dimethylformamide (1.15 mL) was added DIPEA (50.2 μL, 288 μmol) and HATU (43.9 mg, 115 μmol). The mixture stirred at ambient temperature for 3 hours. The reaction mixture was diluted with EtOAc, washed with 1:1 water: saturated aqueous NH₄Cl (2×) then with brine (2×). The organic layer was dried over anhydrous Na₂SO₄, filtered and concentrated under reduced pressure to give the crude product and purified by column chromatography (1-4% MeOH in DCM). The product was further purified via Prep HPLC (Isco ACCQ Prep, Gemini column NX—C18, 250×21 mm, 20 ml/min, 45 minute separation, 20 to 75% H2O/ACN with 0.1% Formic acid buffer) to provide (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-18-carbonitrile (28 mg, 32.5 μmol). m/z (ESI-pos) M+1=862.5; ¹H NMR (400 MHz, CHLOROFORM-D) δ 11.22 (d, J=14.5 Hz, 1H), 7.65-7.36 (m, 6H), 7.31-7.26 (m, 1H), 7.24-7.09 (m, 3H), 6.76-6.65 (m, 1H), 6.44-6.13 (m, 1H), 5.83-4.44 (m, 1H), 3.90-3.79 (m, 2H), 3.69-3.24 (m, 1H), 3.14 (ddd, J=17.3, 12.1, 5.7 Hz, 1H), 3.09-2.82 (m, 3H), 2.69-2.38 (m, 1H), 1.91-1.71 (m, 8H), 1.68-1.56 (m, 4H), 1.30 (dd, J=24.5, 3.0 Hz, 10H), 1.22-1.00 (m, 4H); VT NMR @125° C.—¹H NMR (400 MHz, TETRACHLOROETHANE-D) δ 11.02 (d, J=42.2 Hz, 1H), 7.66-7.47 (m, 5H), 7.37 (d, J=8.0 Hz, 1H), 7.33-7.23 (m, 1H), 7.17 (t, J=9.1 Hz, 2H), 6.72 (d, J=11.1 Hz, 1H), 6.45-6.22 (m, 1H), 5.73 (s, 1H), 4.55 (br s, 1H), 4.01-3.74 (m, 2H), 3.62 (s, 1H), 3.27-2.95 (m, 4H), 2.87 (s, 1H), 2.70 (t, J=12.2 Hz, 1H), 2.56-2.41 (m, 1H), 2.00-1.73 (m, 6H), 1.73-1.49 (m, 6H), 1.42-1.29 (m, 9H), 1.22 (s, 3H).

### Example 54—Synthesis of Compound 54

### Synthetic Scheme

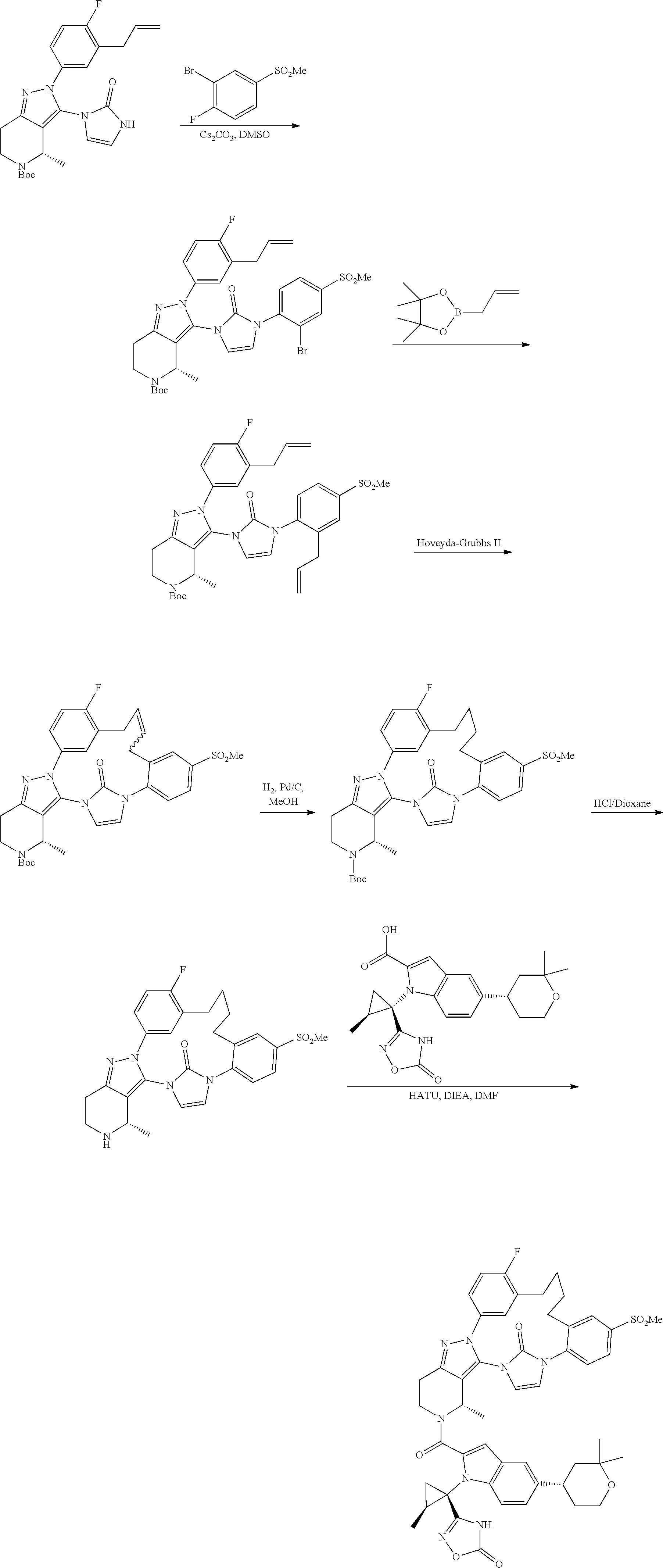

### Procedure for Synthesis of Compound 54

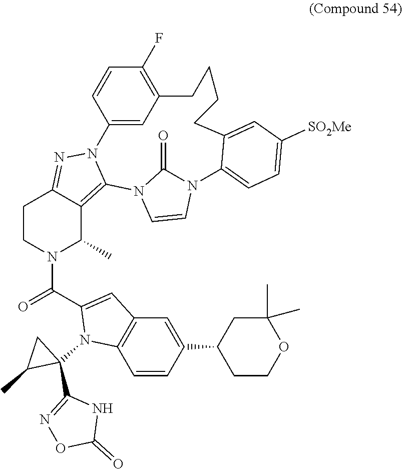

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-18-mesyl-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a solution of tert-butyl (S)-2-(3-allyl-4-fluorophenyl)-4-methyl-3-(2-oxo-2,3-dihydro-1H-imidazol-1-yl)-1,2,5-triaza-2,4,6,7-tetrahydro-5H-indene-5-carboxylate (153 mg, 337 μmol) in DMSO (2 mL) was added dicaesium carbonate (220 mg, 675 μmol). The mixture was stirred at room temperature for 5 min, followed by the addition of 2-bromo-1-fluoro-4-mesylbenzene (256 mg, 1.01 mmol). The reaction mixture was heated at 100° C. for 30 min, cooled to room temperature, and then diluted with H₂O and saturated aqueous NH₄Cl. The reaction mixture was extracted with EtOAc (3×). The combined organic layers were washed with brine then dried over Na₂SO₄, filtered and concentrated in vacuo. The residue was purified by flash column chromatography (20-70% EtOAc in Heptane), to give tert-butyl (S)-2-(3-allyl-4-fluorophenyl)-3-[3-(2-bromo-4-mesylphenyl)-2-oxo-2,3-dihydro-1H-imidazol-1-yl]-4-methyl-1,2,5-triaza-2,4,6,7-tetrahydro-5H-indene-5-carboxylate (226 mg, 329 μmol). m/z (ESI-pos) M+1=686.3, 688.3.

Step B: ert-butyl(S)-2-(3-allyl-4-fluorophenyl)-3-[3-(2-bromo-4-mesylphenyl)-2-oxo-2,3-dihydro-1H-imidazol-1-yl]-4-methyl-1,2,5-triaza-2,4,6,7-tetrahydro-5H-indene-5-carboxylate (204 mg, 297 μmol), bis[2-(diphenylphosphino)-2,4-cyclopentadien-1-ide]-dichloro-palladamethane-dichloromethane-iron (1/1/1/1) (60.7 mg, 74.3 μmol), and dipotassium carbonate (123 mg, 891 μmol) were dissolved in 1,4-dioxane (3.0 mL) and water (0.5 mL), then 2-allyl-4,4,5,5-tetramethyl-1,3,2-dioxaborolane (167 μL, 891 μmol) was added. The mixture was degassed with N₂ for 5 min then heated to 80° C. for 4 h. Then additional 2-allyl-4,4,5,5-tetramethyl-1,3,2-dioxaborolane (167 μL, 891 μmol), dipotassium carbonate (123 mg, 891 μmol) and bis[2-(diphenylphosphino)-2,4-cyclopentadien-1-ide]-dichloro-palladamethane-dichloromethane-iron (1/1/1/1) (60.7 mg, 74.3 μmol) were added and the mixture stirred for another 7 hours. The reaction mixture was cooled down to room temperature and diluted with water. The mixture was extracted with ethyl acetate (3×). The combined organic layers were washed with brine solution, dried over anhydrous Na₂SO₄, filtered and concentrated. The crude compound was purified by column chromatography (30-60% EtOAc/Heptane) to give tert-butyl (S)-2-(3-allyl-4-fluorophenyl)-3-[3-(2-allyl-4-mesylphenyl)-2-oxo-2,3-dihydro-1H-imidazol-1-yl]-4-methyl-1,2,5-triaza-2,4,6,7-tetrahydro-5H-indene-5-carboxylate (94 mg, 145 μmol). m/z (ESI-pos) M+1=648.4

Steps C, D, E and F were performed as described in Example 53 to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-18-mesyl-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (50 mg, 54.6 μmol). m/z (ESI-pos) M+1=915.5; 1H NMR (400 MHz, CHLOROFORM-D) δ 11.22 (d, J=15.5 Hz, 1H), 7.95-7.42 (m, 6H), 7.29-7.23 (obs m, 1H), 7.15 (dt, J=11.2, 8.9 Hz, 2H), 6.74-6.67 (m, 1H), 6.44-6.26 (m, 1H), 5.83-4.43 (m, 1H), 3.90-3.79 (m, 2H), 3.68-3.31 (m, 1H), 3.16-2.98 (m, 6H), 2.57 (dd, J=43.3, 11.3 Hz, 1H), 1.74 (d, J=18.7 Hz, 14H), 1.36-1.16 (m, 14H).

### Example 55—Synthesis of Compound 55

### Procedure for Synthesis of Compound 55

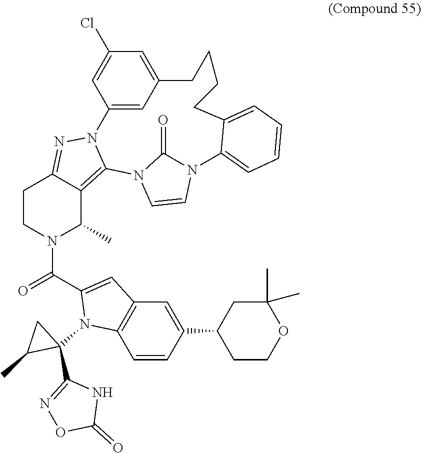

### (S)-27-chloro-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 55 was synthesized following the procedure of Example 34, with (3-bromo-4-fluorophenyl)hydrazine in Step B being replaced with commercially available (3-bromo-5-chlorophenyl)hydrazine hydrogen chloride (1.0 eq) and N-ethyldiisopropylamine (1.5 eq). This provided(S)-27-chloro-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (11 mg, 12.9 μmol). m/z (ESI-pos) M+1=853.5; ¹H NMR (400 MHz, CHLOROFORM-D) δ 11.27-11.21 (m, 1H), 7.60-7.54 (m, 1H), 7.54-7.44 (m, 2H), 7.38-7.26 (m, 4H), 7.20-6.95 (m, 3H), 6.78-6.64 (m, 1H), 6.46-5.92 (m, 2H), 5.85-5.21 (m, 1H), 4.91-4.42 (m, 1H), 3.88-3.80 (m, 2H), 3.67-3.55 (m, 1H), 3.20-2.96 (m, 4H), 2.79 (s, 2H), 2.73-2.65 (m, 2H), 2.50-2.31 (m, 1H), 1.88 (dt, J=21.5, 6.2 Hz, 2H), 1.81-1.65 (m, 6H), 1.60 (dd, J=11.8, 6.7 Hz, 4H), 1.31 (d, J=23.9 Hz, 6H), 1.26-1.18 (m, 3H); ¹H NMR (400 MHz, DMSO-D6 at 120° C.) δ 11.47 (s, 1H), 7.54-7.44 (m, 2H), 7.39 (t, J=8.3 Hz, 1H), 7.35-7.16 (m, 6H), 6.97-6.70 (m, 4H), 5.55 (s, 1H), 4.46 (s, 1H), 3.79-3.64 (m, 2H), 3.54 (t, J=11.8 Hz, 1H), 3.08-2.38 (m, 7H), 1.81-1.40 (m, 14H), 1.31-1.13 (m, 9H).

### Example 56—Synthesis of Compound 56

### Synthetic Scheme

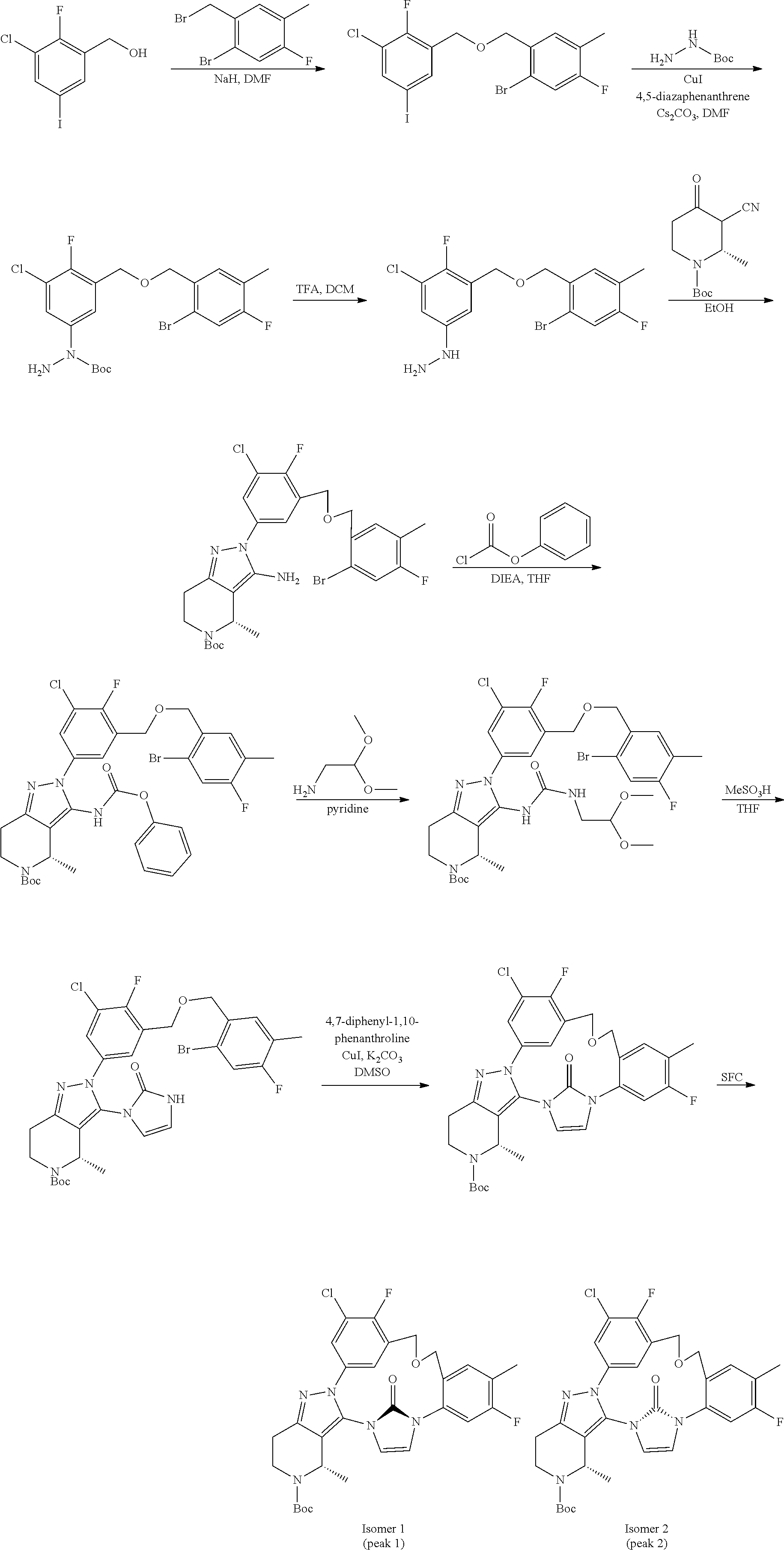

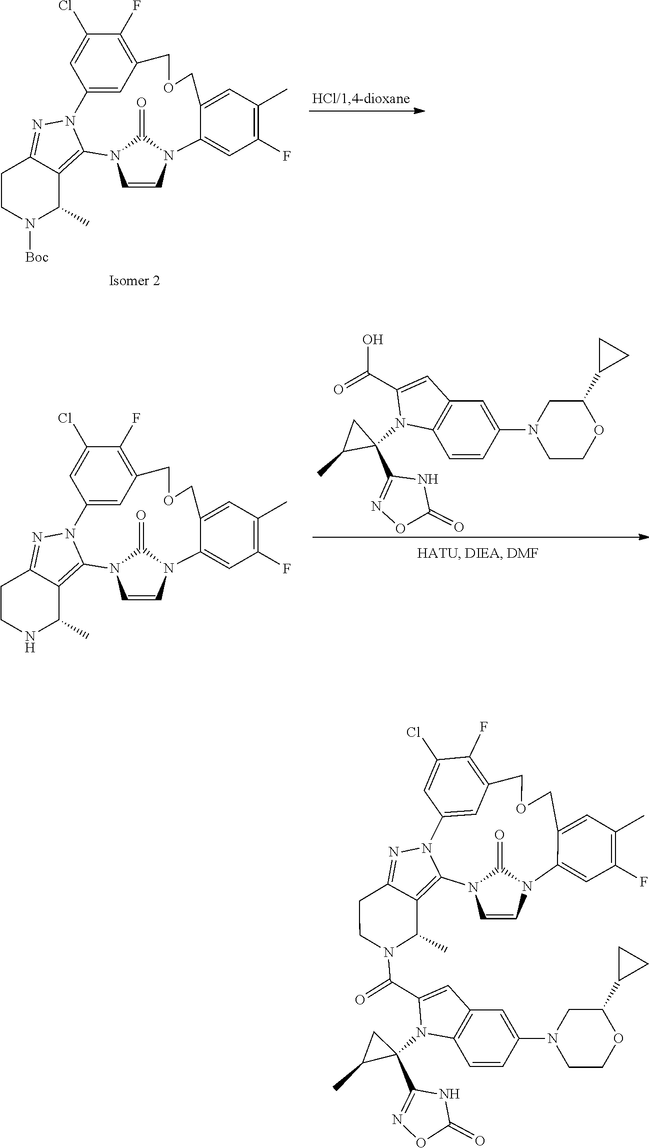

### Procedure for Synthesis of Compound 56

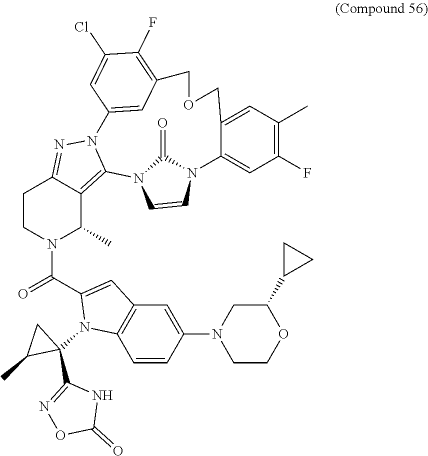

### (8S,10M)-26-chloro-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (3-chloro-2-fluoro-5-iodophenyl) methanol (2.9 g, 10.1 mmol) in DMF (30 mL) was added NaH 60% w/w (607 mg, 15.2 mmol) at 0° C. under N₂. The mixture was stirred at 0° C. for 30 minutes and then 4-bromo-5-(bromomethyl)-2-fluorotoluene (2.85 g, 10.1 mmol) was added. The resulting mixture was allowed to warm to room temperature and was stirred for 2 hours, and then the reaction was quenched with sat. aq. NH₄Cl, diluted with ethyl acetate, washed with water (×2) and the aqueous layer was extracted with ethyl acetate (×2). The combined organic layers were washed with brine solution, dried over anhydrous Na₂SO₄, and evaporated under reduced pressure to give the crude compound, which was purified by column chromatography (0-10% EtOAc in PE) to give 3-{[(2-bromo-4-fluoro-5-tolyl)methoxy]methyl}-1-chloro-2-fluoro-5-iodobenzene (2.5 g, 4.62 mmol) as a white solid. ¹H NMR (400 MHz, CDCl₃) δ 7.72-7.64 (m, 2H), 7.28 (d, J=8.4 Hz, 1H), 7.24 (d, J=9.2 Hz, 1H), 4.62 (s, 2H), 4.59 (s, 2H), 2.25 (d, J=1.7 Hz, 3H).

Step B: To a solution of 3-{[(2-bromo-4-fluoro-5-tolyl)methoxy]methyl}-1-chloro-2-fluoro-5-iodobenzene (2.5 g, 5.13 mmol), and tert-butyl carbazate (881 mg, 6.67 mmol) in DMF (48.2 mL) were added Cs₂CO₃ (2.34 g, 7.18 mmol), 4,5-diazaphenanthrene (370 mg, 2.05 mmol) and copper (I) iodide (97.7 mg, 513 μmol) at room temperature under N₂. The mixture was stirred at 80° C. under N₂ for 16 hours. The reaction mixture was cooled to room temperature and diluted with water, and then extracted with ethyl acetate (×2). The combined organics were washed with brine (×2), dried over Na₂SO₄ and concentrated in vacuo, and the crude product was purified by column chromatography (0-50% ethyl acetate in PE) to give tert-butyl 2-(5-{[(2-bromo-4-fluoro-5-tolyl)methoxy]methyl}-3-chloro-4-fluorophenyl) carbazate (2.03 g, 3.72 mmol) as a yellow solid. m/z (ESI-pos) [M+H-tBu]=435.0

Step C: To a solution of tert-butyl 2-(5-{[2-(o-bromophenyl) ethoxy]methyl}-3-chloro-4-fluorophenyl) carbazate (2.03 g, 4.28 mmol) in DCM (14 mL) was added TFA (4.88 g, 42.8 mmol) at 0° C. The reaction mixture was stirred at room temperature for 2 hours, and then the reaction was quenched with 2M aq. Na₂CO₃ at 0° C. The solution was then diluted with DCM and washed with water (3×), and the aqueous layer was extracted with DCM (2×). The combined organic layers were washed with brine solution, dried over anhydrous Na₂SO₄, and evaporated under reduced pressure to give the crude compound (5-{[2-(o-bromophenyl)ethoxy]methyl}-3-chloro-4-fluorophenyl)hydrazine (1.7 g, 2.27 mmol) as a yellow oil, which was used to next step directly. m/z (ESI-pos) [M+1]=391.0

Step D: To a solution of (5-{[(2-bromo-4-fluoro-5-tolyl)methoxy]methyl}-3-chloro-4-fluorophenyl)hydrazine (1.68 g, 4.28 mmol) in ethanol (20 mL) was added tert-butyl(2S)-3-cyano-2-methyl-4-oxo-1-piperidinecarboxylate (1.02 g, 4.28 mmol) at room temperature under N₂. The reaction mixture was then heated at 80° C. for 16 hours under N₂ and then the mixture was directly evaporated under reduced pressure to give the crude compound, which was purified by column chromatography (0-100% ethyl acetate in PE) to give tert-butyl (S)-3-amino-2-(5-{[(2-bromo-4-fluoro-5-tolyl)methoxy]methyl}-3-chloro-4-fluorophenyl)-4-methyl-1,2,5-triaza-2,4,6,7-tetrahydro-5H-indene-5-carboxylate (1.84 g, 2.2 mmol) as a yellow oil. m/z (ESI-pos) [M+1]=611.1

Step E: To a solution of tert-butyl (S)-3-amino-2-(5-{[(2-bromo-4-fluoro-5-tolyl)methoxy]methyl}-3-chloro-4-fluorophenyl)-4-methyl-1,2,5-triaza-2,4,6,7-tetrahydro-5H-indene-5-carboxylate (1.84 g, 3.01 mmol) and DIEA (1.17 g, 9.02 mmol) in THF (25 mL) was added benzenyl chloroformate (942 mg, 6.01 mmol) at 0° C. Then the mixture was warmed to room temperature and stirred at for 3 hours. The reaction mixture was directly concentrated under reduced pressure to give crude tert-butyl (S)-2-(5-{[(2-bromo-4-fluoro-5-tolyl)methoxy]methyl}-3-chloro-4-fluorophenyl)-4-methyl-3-[phenyl(oxycarbonylamino)]-1,2,5-triaza-2,4,6,7-tetrahydro-5H-indene-5-carboxylate (2.2 g, 2.4 mmol) as a yellow oil, which was used directly in the next step without any further purification. m/z (ESI-pos) [M+1]=731.0

Step F: To a solution of tert-butyl (S)-2-(5-{[(2-bromo-4-fluoro-5-tolyl)methoxy]methyl}-3-chloro-4-fluorophenyl)-4-methyl-3-[phenyl(oxycarbonylamino)]-1,2,5-triaza-2,4,6,7-tetrahydro-5H-indene-5-carboxylate (2.2 g, 3 mmol) in pyridine (20 mL) was added 2,2-dimethoxyethylamine (947 mg, 9 mmol) at 0° C. The reaction mixture was allowed to warm to room temperature and stirred for 2 hours, then the reaction mixture was concentrated to give the crude product, which was purified by column chromatography (0-100% ethyl acetate in PE) to give tert-butyl (S)-2-(5-{[(2-bromo-4-fluoro-5-tolyl)methoxy]methyl}-3-chloro-4-fluorophenyl)-3-[3-(2,2-dimethoxyethyl)ureido]-4-methyl-1,2,5-triaza-2,4,6,7-tetrahydro-5H-indene-5-carboxylate (1.5 g, 1.61 mmol) as a yellow solid. m/z (ESI-pos) [M+1]=742.2

Step G: To a solution of tert-butyl (S)-2-(5-{[(2-bromo-4-fluoro-5-tolyl)methoxy]methyl}-3-chloro-4-fluorophenyl)-3-[3-(2,2-dimethoxyethyl)ureido]-4-methyl-1,2,5-triaza-2,4,6,7-tetrahydro-5H-indene-5-carboxylate (1.5 g, 1.61 mmol) in THF (30 mL) was added methanesulfonic acid (233 mg, 2.42 mmol) at room temperature. The mixture was stirred at 60° C. for 2 hours and then cooled to room temperature, and the pH was adjusted to about 9 with aqueous K₃PO₄. Di-tert-butyl dicarbonate (352 mg, 1.61 mmol) was added, and the resulting mixture was stirred at room temperature for 16 hours. The reaction mixture was diluted with ethyl acetate, washed with water (3×), and the aqueous layer was extracted with ethyl acetate (3×). The combined organic layers were washed with brine solution, dried over anhydrous Na₂SO₄, filtered, and the filtrate was evaporated under reduced pressure. The crude compound was purified by column chromatography (0-100% ethyl acetate in PE) to give tert-butyl (S)-2-(5-{[(2-bromo-4-fluoro-5-tolyl)methoxy]methyl}-3-chloro-4-fluorophenyl)-4-methyl-3-(2-oxo-2,3-dihydro-1H-imidazol-1-yl)-1,2,5-triaza-2,4,6,7-tetrahydro-5H-indene-5-carboxylate (1.05 g, 1.47 mmol) as a yellow solid. m/z (ESI-pos) [M+1]=678.0

Step H: To a solution of tert-butyl (S)-2-(5-{[(2-bromo-4-fluoro-5-tolyl)methoxy]methyl}-3-chloro-4-fluorophenyl)-4-methyl-3-(2-oxo-2,3-dihydro-1H-imidazol-1-yl)-1,2,5-triaza-2,4,6,7-tetrahydro-5H-indene-5-carboxylate (1.02 g, 1.5 mmol) in DMSO (15 mL) was added K₂CO₃ (519 mg, 3.76 mmol), 4,7-diphenyl-1,10-phenanthroline (499 mg, 1.5 mmol), and copper (I) iodide (215 mg, 1.13 mmol) at room temperature. The mixture was stirred at 120° C. for 16 hours under N₂ protection. The reaction was then cooled to room temperature, diluted with ethyl acetate, washed with water (3×), and then the aqueous layer was extracted with ethyl acetate (3×). The combined organic layers were washed with brine solution, dried over anhydrous Na₂SO₄, and evaporated under reduced pressure to give the crude compound, which was purified by column chromatography (0-100% ethyl acetate in PE) to give tert-butyl (2⁴S)-1⁵-chloro-1⁴,4⁵-difluoro-2⁴,4⁴-dimethyl-3²-oxo-2⁴,2⁵,2⁶,2⁷,3²,3³-hexahydro-2²H,3¹H-6-oxa-2(2,3)-pyrazolo[4,3-c]pyridina-3(1,3)-imidazola-1(1,3),4(1,2)-dibenzenacycloheptaphane-2⁵-carboxylate (250 mg, 376 μmol) as a yellow solid. m/z (ESI-pos) [M+1]=598.1

Step I: tert-butyl (2⁴S)-1⁵-chloro-1⁴,4⁵-difluoro-2⁴,4⁴-dimethyl-3²-oxo-2⁴,2⁵,2⁶,2⁷,3²,3³ hexahydro-2²H,3¹H-6-oxa-2(2,3)-pyrazolo[4,3-c]pyridina-3(1,3)-imidazola-1(1,3),4(1,2)-dibenzenacycloheptaphane-2⁵-carboxylate (250 mg, 376 μmol) was separated by chiral supercritical fluid chromatography [Apparatus: SFC 80; Column: Daicel CHIRALCEL ID, 250 mm*30 mm; Mobile Phase: CO₂/MeOH [0.2% NH₃ (7M Solution in MeOH)]=60/40; Flow rate: 80 g/min; Temp: 35° C.; Wavelength: 214 nm] to afford tert-butyl (2⁴S,3¹S,3³R)-1⁵-chloro-1⁴,4⁵-difluoro-2⁴,4⁴-dimethyl-3²-oxo-2⁴,2⁵,2⁶,2⁷,3²,3³-hexahydro-2²H,3¹H-6-oxa-2(2,3)-pyrazolo[4,3-c]pyridina-3(1,3)-imidazola-1(1,3),4(1,2)-dibenzenacycloheptaphane-2⁵-carboxylate (isomer 1, 28.48 mg) and tert-butyl (2⁴S,3¹R,3³S)-1⁵-chloro-1⁴,4⁵-difluoro-2⁴,4⁴-dimethyl-3²-oxo-2⁴,2⁵,2⁶,2⁷,3²,3³-hexahydro-2²H,3¹H-6-oxa-2(2,3)-pyrazolo[4,3-c]pyridina-3(1,3)-imidazola-1(1,3),4(1,2)-dibenzenacycloheptaphane-2⁵-carboxylate (isomer 2, 118.96 mg) as white solids. (The absolute configuration was unknown, and assigned arbitrarily). m/z (ESI-pos) [M+1]=598.2

Step J: A solution of isomer 2 from the SFC separation (27 mg, 45.1 μmol) in 1.3 mL of 4M HCl/1,4-dioxane was stirred for 1 hour at room temperature. The solution was then concentrated and taken forward as the crude product. m/z (ESI-pos) [M+1]=498.3

Step K: A solution of (8S,10M)-26-chloro-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (21 mg, 39.3 μmol), DIEA (20.5 μL, 118 μmol), 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid (16.7 mg, 39.3 μmol), and HATU (22.4 mg, 58.9 μmol) in 0.2 mL of DMF was stirred overnight at room temperature. The reaction was then purified directly by reverse-phase HPLC (5-95% MeCN/water, 0.1% FA), and the product was concentrated to give (8S,10M)-26-chloro-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (9 mg, 9.95 μmol) as a white solid (the absolute configuration was unknown, and the configuration of the product was arbitrarily assigned). m/z (ESI-pos) [M+1]=904.6; ¹H NMR (400 MHz, chloroform-D) δ 11.26 (d, J=23.4 Hz, 1H), 7.98 (s, 1H), 7.77-7.68 (m, 1H), 7.59-7.50 (m, 1H), 7.37 (d, J=7.9 Hz, 1H), 7.14 (tt, J=7.5, 2.6 Hz, 2H), 6.76-6.61 (m, 1H), 6.44 (d, J=3.1 Hz, 1H), 6.34-6.22 (m, 1H), 5.78 (q, J=6.7 Hz, 1H), 4.86 (d, J=5.2 Hz, 1H), 4.74 (q, J=14.9 Hz, 2H), 4.49 (dd, J=13.8, 5.0 Hz, 1H), 4.25 (d, J=9.3 Hz, 1H), 4.11-4.01 (m, 2H), 3.79 (td, J=11.4, 2.6 Hz, 1H), 3.67-3.55 (m, 1H), 3.51 (d, J=11.6 Hz, 1H), 3.34 (d, J=11.6 Hz, 1H), 3.18-3.06 (m, 1H), 3.02 (dd, J=16.0, 3.9 Hz, 1H), 2.97-2.90 (m, 1H), 2.85 (td, J=11.6, 3.2 Hz, 1H), 2.76-2.66 (m, 1H), 2.35-2.28 (m, 3H), 1.88 (d, J=5.9 Hz, 1H), 1.64 (d, J=6.7 Hz, 3H), 1.59-1.48 (m, 1H), 1.23-1.11 (m, 3H), 0.95 (dtd, J=13.2, 8.3, 4.9 Hz, 1H), 0.67-0.52 (m, 2H), 0.50-0.41 (m, 1H), 0.29 (dq, J=9.6, 4.8 Hz, 1H).

### Example 57—Synthesis of Compound 57

### Synthetic Scheme

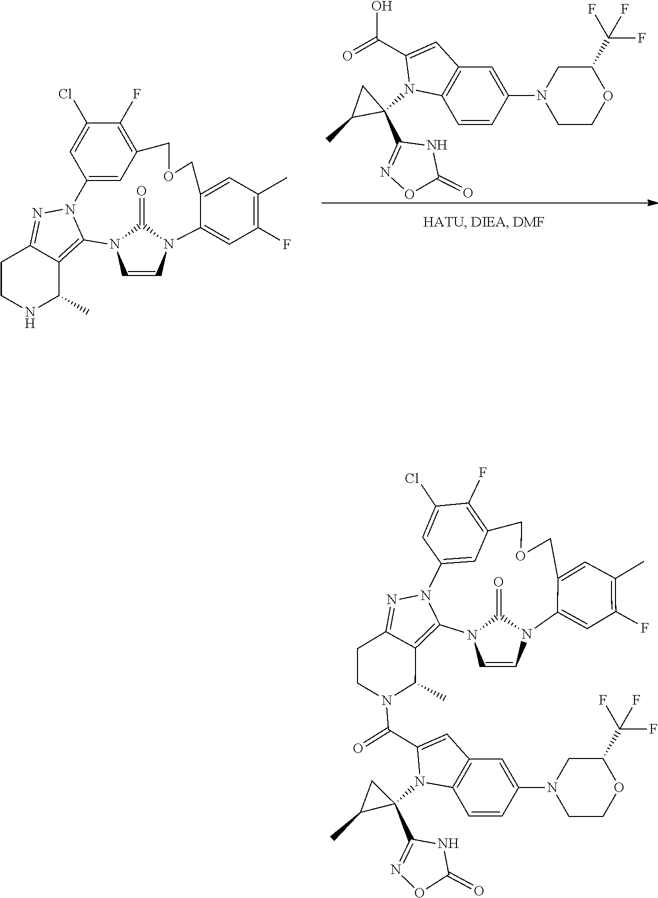

### Procedure for Synthesis of Compound 57

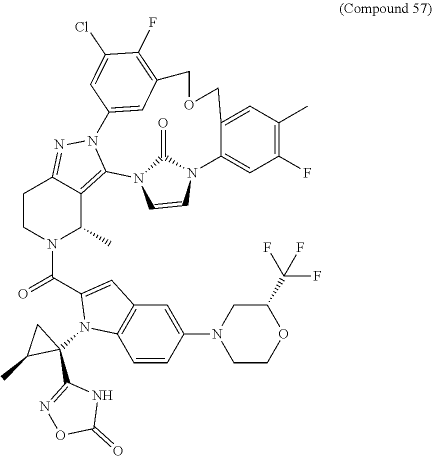

### (8S,10M)-26-chloro-17,25-difluoro-8,18-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: A solution of (8S,10M)-26-chloro-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (24 mg, 44.9 μmol), 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid (22.3 mg, 49.4 μmol), DIEA (23.5 μL, 135 μmol), and HATU (25.6 mg, 67.4 μmol) in 0.2 mL of DMF was stirred for overnight at 50 C. The reaction was then purified directly by reverse-phase HPLC (5-95% MeCN/water, 0.1% FA), and the product was concentrated to give (8S,10M)-26-chloro-17,25-difluoro-8,18-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (17 mg, 18.2 μmol) as a white solid (the absolute configuration was unknown, and the configuration of the product was arbitrarily assigned). m/z (ESI-pos) [M+1]=932.6; ¹H NMR (400 MHz, chloroform-D) δ 11.26 (s, 1H), 7.74 (dd, J=6.1, 2.7 Hz, 1H), 7.57 (t, J=8.0 Hz, 1H), 7.37 (d, J=7.9 Hz, 1H), 7.14 (dtd, J=11.1, 5.7, 2.6 Hz, 3H), 6.70 (d, J=34.8 Hz, 1H), 6.44 (d, J=3.0 Hz, 1H), 6.32-6.22 (m, 1H), 5.78 (q, J=6.6 Hz, 1H), 4.74 (q, J=14.9 Hz, 2H), 4.47 (dd, J=13.8, 5.0 Hz, 1H), 4.26 (d, J=9.4 Hz, 1H), 4.22-4.04 (m, 4H), 3.90 (td, J=11.4, 2.6 Hz, 1H), 3.67-3.50 (m, 2H), 3.36 (d, J=12.1 Hz, 1H), 3.19-2.82 (m, 5H), 2.35-2.28 (m, 3H), 1.90 (t, J=6.1 Hz, 1H), 1.64 (d, J=6.7 Hz, 3H), 1.56-1.48 (m, 2H), 1.21-1.11 (m, 3H).

### Example 58—Synthesis of Compound 58

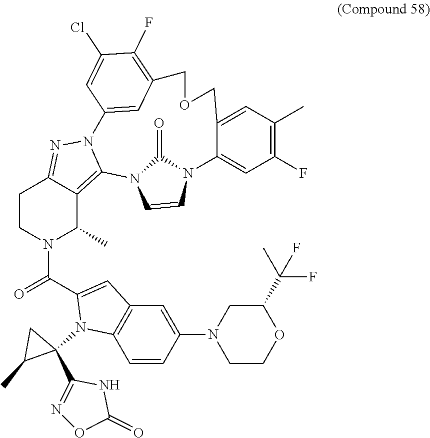

### (8S,10M)-26-chloro-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 58 was synthesized following the procedure of Example 57, with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid being replaced with 5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolecarboxylic acid. m/z (ESI-pos) [M+1]=932.6; 1H NMR (400 MHz, chloroform-D) δ 11.28 (s, 1H), 7.75 (s, 1H), 7.57 (d, J=8.7 Hz, 1H), 7.38 (d, J=7.8 Hz, 1H), 7.20-7.02 (m, 4H), 6.66 (d, J=1.8 Hz, 1H), 6.48-6.42 (m, 1H), 6.34-6.28 (m, 1H), 5.79 (d, J=7.0 Hz, 1H), 4.75 (q, J=15.4 Hz, 2H), 4.49 (d, J=14.0 Hz, 1H), 4.27 (d, J=9.4 Hz, 1H), 4.15-4.05 (m, 2H), 3.88 (t, J=11.2 Hz, 2H), 3.61 (dd, J=23.6, 11.9 Hz, 2H), 3.38 (d, J=12.1 Hz, 1H), 3.08 (dd, J=34.5, 13.9 Hz, 2H), 2.88 (t, J=11.7 Hz, 1H), 2.78 (t, J=11.0 Hz, 1H), 2.33 (d, J=8.5 Hz, 3H), 1.90 (t, J=5.9 Hz, 1H), 1.68-1.62 (m, 3H), 1.60-1.49 (m, 2H), 1.28-1.17 (m, 3H).

### Example 59—Synthesis of Compound 59

### Synthetic Scheme

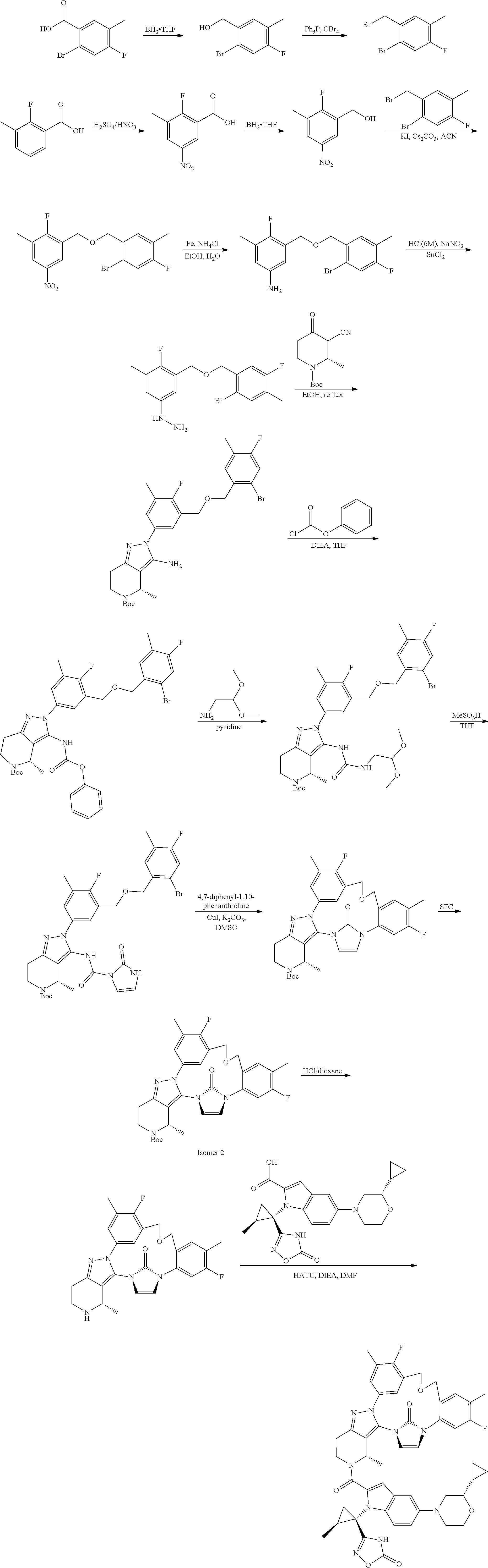

### Procedure for Synthesis of Compound 59

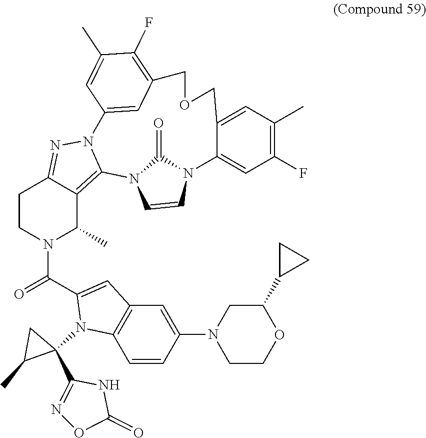

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-8,18,26-trimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of 4-bromo-6-fluoro-3-toluic acid (10 g, 42.9 mmol) in 200 mL of THF was added borane-methyl sulfide complex (1.0 M in THF, 85.8 mL, 85.8 mmol) at 0° C. The reaction was stirred at room temperature for 16 hours under N₂. MeOH (30 ml) was added dropwise into the reaction mixture at 0° and the resulting mixture was stirred at 60° C. for 1 hour. The mixture was cooled to room temperature and diluted with brine, and then extracted with ethyl acetate. The combined organics were washed with brine, dried over Na₂SO₄ and concentrated in vacuo. The crude product was purified by silica gel chromatography (0-25% EtOAc in pet. ether) to give (2-bromo-4-fluoro-5-tolyl) methanol (9.1 g, 37.4 mmol) as a white solid. ¹H NMR (400 MHz, CDCl₃) δ=7.27 (d, J=8.0, ¹H), 7.20 (d, J=8.8, ¹H), 4.65 (s, 2H), 2.30 (s, 1H), 2.22 (d, J=1.6, 3H).

Step B: To a solution of (2-bromo-4-fluoro-5-tolyl)methanol (9.1 g, 39.5 mmol) and PPh₃ (10.4 g, 39.5 mmol) in 100 mL of DCM was added CBr₄ (14.4 g, 43.4 mmol) at 0° C. The reaction was stirred at room temperature for 16 hours under N₂ and then diluted with brine and washed with DCM. The combined organics were concentrated in vacuo and the crude product was purified by silica gel chromatography (0-5% EtOAc in pet. ether) to give 4-bromo-5-(bromomethyl)-2-fluorotoluene (9.2 g, 29.4 mmol) as a white solid. ¹H NMR (400 MHz, CDCl₃) δ=7.28 (d, J=7.6, ¹H), 7.23 (d, J=8.8, ¹H), 4.54 (s, 2H), 2.22 (s, 3H).

Step C: A solution of 2-fluoro-3-toluic acid (10 g, 64.9 mmol) in sulfuric acid (60 mL, 64.9 mmol) was cooled to −10° C., then nitric acid (9.02 g, 97.3 mmol) was added into the solution and the reaction mixture was stirred at 0° C. for 3 hours under N₂. The reaction mixture was poured into ice water, then extracted with EA. The combined EA layers were washed with brine, dried over Na₂SO₄ and concentrated in vacuo to give 2-fluoro-5-nitro-3-toluic acid (13.1 g, 52.6 mmol) as a yellow solid, which was used in next step directly.

Step D: To a solution of 2-fluoro-5-nitro-3-toluic acid (11.6 g, 58.3 mmol) in 240 mL of THF was added borane-methyl sulfide complex (2.0M in THF, 116 mL, 117 mmol) at 0° C. The mixture was stirred at 50° C. for 3 hours under N₂. MeOH was added dropwise at 0° C. and then the mixture was stirred at 60° C. for 1 hour. The mixture was cooled to room temperature, diluted with brine and washed with EtOAc. The combined organics were concentrated in vacuo and the crude product was purified by silica gel chromatography (0-25% EtOAc in pet. ether) to give (2-fluoro-5-nitro-3-tolyl)methanol (9.4 g, 45.7 mmol) as a white solid. ¹H NMR (400 MHz, CDCl₃) δ=8.22 (dd, J=5.6, 2.8, ¹H), 8.04 (dd, J=6.0, 2.8, 1H), 4.82 (s, 2H), 2.37 (d, J=2.0, 3H).

Step E: To a solution of (2-fluoro-5-nitro-3-tolyl)methanol 90% w/w (5 g, 24.3 mmol) in 100 mL of acetonitrile was added 4-bromo-5-(bromomethyl)-2-fluorotoluene (7.54 g, 26.7 mmol), Cs₂CO₃ (19.8 g, 60.8 mmol) and KI (807 mg, 4.86 mmol). The mixture was stirred at 40° C. for 16 hours and then cooled to room temperature, diluted with water washed with EtOAc. The combined organics were concentrated in vacuo and the crude product was purified by silica gel chromatography (0-25% EtOAc in pet. ether) to give 3-{[(2-bromo-4-fluoro-5-tolyl)methoxy]methyl}-2-fluoro-5-nitrotoluene (3.3 g, 7.69 mmol) as a white solid. ¹H NMR (400 MHz, CDCl₃) δ=8.25 (dd, J=6.0, 3.2, ¹H), 8.06 (dd, J=6.4, 2.8, ¹H), 7.32 (d, J=8.0, ¹H), 7.25 (d, J=4.4, ¹H), 4.70 (s, 2H), 4.64 (s, 2H), 2.38 (d, J=2.0, 3H), 2.25 (d, J=2.0, 3H).

Step F: To the solution of 3-{[(2-bromo-4-fluoro-5-tolyl)methoxy]methyl}-2-fluoro-5-nitrotoluene (3.3 g, 6.84 mmol) in 60 mL of EtOH and 20 mL of H₂O were added iron (1.91 g, 34.2 mmol) and ammonium chloride (1.83 g, 34.2 mmol). The mixture was stirred at 70° C. for 6 hours. The mixture was cooled to room temperature and diluted with brine and then extracted with EtOAc. The organics were concentrated and purified by silica gel chromatography (0-25% EtOAc in pet. ether) to give 3-{[(2-bromo-4-fluoro-5-tolyl)methoxy]methyl}-4-fluoro-5-tolylamine (2.1 g, 5.6 mmol) as a yellow oil. m/z (ESI-pos) 356.1/358.1

Step G: To a solution of 3-((2-bromo-4-fluorophenethoxy)methyl)-5-chloroaniline (2.15 g, 5.4 mmol) in aq. HCl (6N, 60 mL) at 0° C. was added an aqueous solution of NaNO₂ (409 mg, 5.93 mmol) in water (10 mL). The mixture was stirred at room temperature for 1 hour, and then a solution of SnCl₂ (2.25 g, 11.9 mmol) in aq. HCl (6N, 20 mL) was added dropwise. The resulting mixture was stirred for 1 hour. Then the mixture was extracted with EtOaC, and the organics were dried with sodium sulfate, filtered, and concentrated in vacuo to get (3-{[(2-bromo-4-fluoro-5-tolyl)methoxy]methyl}-4-fluoro-5-tolyl) hydrazine hydrochloride (2 g, 5.39 mmol) as a yellow oil, which was used in next step directly. m/z (ESI-pos) 373.1.

Step H: A solution of (3-{[(2-bromo-4-fluoro-5-tolyl)methoxy]methyl}-4-fluoro-5-tolyl) hydrazine (2 g, 4.31 mmol) and tert-butyl(2S)-3-cyano-2-methyl-4-oxo-1-piperidinecarboxylate (1.03 g, 4.31 mmol) in ethanol (36.9 mL) was stirred at 80° C. for 16 hours under N₂. The mixture was concentrated under reduced pressure to give the crude residue, which was purified by silica gel chromatography (0-40% EtOAc in pet. ether) to give tert-butyl (S)-3-amino-2-(3-{[(2-bromo-4-fluoro-5-tolyl)methoxy]methyl}-4-fluoro-5-tolyl)-4-methyl-1,2,5-triaza-2,4,6,7-tetrahydro-5H-indene-5-carboxylate (1.47 g, 2.24 mmol) as a yellow oil. m/z (ESI-pos) 591.2

Step I: To a solution of tert-butyl (S)-3-amino-2-(3-{[(2-bromo-4-fluoro-5-tolyl)methoxy]methyl}-4-fluoro-5-tolyl)-4-methyl-1,2,5-triaza-2,4,6,7-tetrahydro-5H-indene-5-carboxylate (1.47 g, 2.24 mmol) in THF (20 mL) were added DIEA (1.19 mL, 6.71 mmol) and benzenyl chloroformate (0.7 g, 4.47 mmol) at 0° C. The mixture was stirred at room temperature for 1 hour under N₂ and then directly concentrated to give crude tert-butyl (S)-2-(3-{[(2-bromo-4-fluoro-5-tolyl)methoxy]methyl}-4-fluoro-5-tolyl)-4-methyl-3-[phenyl(oxycarbonylamino)]-1,2,5-triaza-2,4,6,7-tetrahydro-5H-indene-5-carboxylate (1.59 g, 1.79 mmol) as a yellow oil, which was used directly for next step without any further purification. m/z (ESI-pos) 711.2

Step J: To a solution of tert-butyl (S)-2-(3-{[(2-bromo-4-fluoro-5-tolyl)methoxy]methyl}-4-fluoro-5-tolyl)-4-methyl-3-[phenyl(oxycarbonylamino)]-1,2,5-triaza-2,4,6,7-tetrahydro-5H-indene-5-carboxylate (1.59 g, 2.23 mmol) in pyridine (18 mL) was added 2,2-dimethoxyethylamine (705 mg, 6.7 mmol) at room temperature and stirred for 16 hours. This was then directly concentrated to give the crude product, which was purified by silica gel chromatography (0-60% EtOAc in pet. ether) to give tert-butyl (S)-2-(3-{[(2-bromo-4-fluoro-5-tolyl)methoxy]methyl}-4-fluoro-5-tolyl)-3-[3-(2,2-dimethoxyethyl)ureido]-4-methyl-1,2,5-triaza-2,4,6,7-tetrahydro-5H-indene-5-carboxylate (1 g, 1.25 mmol) as a yellow oil. m/z (ESI-pos) 722.2

Step K: To a solution of tert-butyl (S)-2-(3-{[(2-bromo-4-fluoro-5-tolyl)methoxy]methyl}-4-fluoro-5-tolyl)-3-[3-(2,2-dimethoxyethyl)ureido]-4-methyl-1,2,5-triaza-2,4,6,7-tetrahydro-5H-indene-5-carboxylate (1 g, 1.25 mmol) in THF (20 mL) was added methanesulfonic acid (239 mg, 2.49 mmol) at room temperature. The mixture was stirred at 60° C. for 2 hours, and then cooled to room temperature, and the pH was adjusted to about 9 with aqueous K₃PO₄. Then Boc₂O (217 mg, 996 μmol) was added, and the resulting mixture was stirred at room temperature for 16 hours. The mixture was extracted with EtOAc, and the organics were concentrated and purified by silica gel chromatography (0-100% EtOAc in pet. ether) to give tert-butyl (S)-2-(3-{[(2-bromo-4-fluoro-5-tolyl)methoxy]methyl}-4-fluoro-5-tolyl)-4-methyl-3-(2-oxo-2,3-dihydro-1H-imidazol-1-yl)-1,2,5-triaza-2,4,6,7-tetrahydro-5H-indene-5-carboxylate (650 mg, 888 μmol) as a yellow solid. m/z (ESI-pos) 658.2

Step L: To a solution of tert-butyl (S)-2-(3-{[(2-bromo-4-fluoro-5-tolyl)methoxy]methyl}-4-fluoro-5-tolyl)-4-methyl-3-(2-oxo-2,3-dihydro-1H-imidazol-1-yl)-1,2,5-triaza-2,4,6,7-tetrahydro-5H-indene-5-carboxylate (0.6 g, 911 μmol) in DMSO (20 mL) was added CuI (130 mg, 683 μmol), 4,7-diphenyl-1,10-phenanthroline (303 mg, 911 μmol) and K₂CO₃ (315 mg, 2.28 mmol). The mixture was stirred at 120° C. for 16 hours under N₂ and then cooled to room temperature, diluted with water and extracted with EtOAc. The combined organics were concentrated and purified by silica gel chromatography (0-40% EtOAc in pet. ether) to give tert-butyl (2⁴S)-1⁵-chloro-4⁵-fluoro-2⁴-methyl-3²-oxo-2⁴,2⁵,2⁶,2⁷,3²,3³-hexahydro-2²H,3¹H-7-oxa-2(2,3)-pyrazolo[4,3-c]pyridina-3(1,3)-imidazola-1(1,3),4(1,2)-dibenzenacyclooctaphane-25-carboxylate (195 mg, 334 μmol) as a white solid. m/z (ESI-pos) 578.3.

Step M: Tert-butyl (2⁴S)-1⁵-chloro-4⁵-fluoro-2⁴-methyl-3²-oxo-2⁴,2⁵,2⁶,2⁷,3²,3³-hexahydro-2²H,3¹H-7-oxa-2(2,3)-pyrazolo[4,3-c]pyridina-3(1,3)-imidazola-1(1,3),4(1,2)-dibenzenacyclooctaphane-25-carboxylate (195 mg, 334 μmol) was separated by chiral supercritical fluid chromatography [Apparatus: SFC 80, Daicel CHIRALCEL IG, 250 mm×30 mm I.D., 10 μm, Mobile phase: CO₂/MeOH [0.2% NH₃ (7M Solution in MeOH)]=40/60, Flow rate: 80 g/min, Wave length: UV 214 nm, Temperature: 35° C.] to afford tert-butyl (2⁴S,3¹S,3³R)-1⁴,4⁵-difluoro-1⁵,2⁴,4⁴-trimethyl-3²-oxo-2⁴,2⁵,2⁶,2⁷,3²,3³-hexahydro-2²H,3¹H-6-oxa-2(2,3)-pyrazolo[4,3-c]pyridina-3(1,3)-imidazola-1(1,3),4(1,2)-dibenzenacycloheptaphane-2⁵-carboxylate (Isomer 1, 20.04 mg) and tert-butyl (2⁴S,3¹R,3³S)-1⁴,4⁵-difluoro-1⁵,2⁴,4⁴-trimethyl-32-oxo-2⁴,2⁵,2⁶,2⁷,3²,3³-hexahydro-2²H,3¹H-6-oxa-2(2,3)-pyrazolo[4,3-c]pyridina-3(1,3)-imidazola-1(1,3),4(1,2)-dibenzenacycloheptaphane-25-carboxylate (Isomer 2, 92.44 mg) as white solids. Isomer 2: (ESI) calcd for C₄₇H₄₇FN₈O₆ [M+H]+m/z 839.36, found 839.3. ¹H NMR (400 MHz, DMSO) δ 7.50 (d, J=8.4 Hz, 1H), 7.44 (dd, J=6.4, 2.4 Hz, 1H), 7.31 (d, J=10.0 Hz, 1H), 6.97 (dd, J=5.6, 2.8 Hz, 1H), 6.74 (d, J=3.2 Hz, 1H), 6.67 (d, J=3.2 Hz, 1H), 5.17 (q, J=6.4 Hz, 1H), 4.70-4.58 (m, 2H), 4.26 (dd, J=13.6, 4.4 Hz, 1H), 4.22-4.03 (m, 2H), 3.23-3.12 (m, 1H), 2.79-2.63 (m, 2H), 2.31-2.27 (m, 6H), 1.46 (s, 9H), 1.34 (d, J=6.8 Hz, 3H).

Step N: A solution of tert-butyl (8S,10M)-17,25-difluoro-8,18,26-trimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (60 mg, 104 μmol) in 0.5 mL of 4M HCl/dioxane was stirred for 1 hour. The solution was then concentrated and taken forward as the crude product. m/z (ESI-pos) [M+1]=478.3

Step O: A solution of (8S,10M)-17,25-difluoro-8,18,26-trimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (18 mg, 35 μmol), DIEA (18.3 μL, 105 μmol), 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid (14.9 mg, 35 μmol), and HATU (20 mg, 52.5 μmol) in 0.2 mL of DMF was stirred overnight. The reaction was then purified by reverse-phase chromatography (5-95% MeCN/water, 0.1% FA), and the product was concentrated to give (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-8,18,26-trimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (15 mg, 17 μmol). m/z (ESI-pos) [M+1]=884.7; ¹H NMR (400 MHz, chloroform-D) δ 11.30 (d, J=21.2 Hz, 1H), 7.99 (s, 1H), 7.55 (t, J=9.4 Hz, 1H), 7.45 (dd, J=6.3, 2.8 Hz, 1H), 7.37 (t, J=7.8 Hz, 1H), 7.20-6.99 (m, 2H), 6.77-6.63 (m, 1H), 6.38 (d, J=3.0 Hz, 1H), 6.22 (dd, J=22.0, 3.0 Hz, 1H), 5.78 (q, J=6.6 Hz, 1H), 4.71 (q, J=14.8 Hz, 2H), 4.47 (dd, J=13.8, 5.0 Hz, 1H), 4.19 (d, J=9.4 Hz, 1H), 4.11-3.99 (m, 2H), 3.80 (td, J=11.4, 2.4 Hz, 1H), 3.68-3.56 (m, 1H), 3.52 (d, J=11.6 Hz, 1H), 3.40-3.29 (m, 1H), 3.20-2.85 (m, 3H), 2.73 (dd, J=11.6, 10.1 Hz, 1H), 2.36-2.27 (m, 6H), 1.94-1.76 (m, 1H), 1.65 (d, J=6.7 Hz, 3H), 1.59-1.47 (m, 1H), 1.16 (dd, J=29.3, 6.0 Hz, 3H), 1.01-0.83 (m, 1H), 0.68-0.52 (m, 2H), 0.46 (dq, J=9.2, 4.8 Hz, 1H), 0.29 (dq, J=9.5, 4.8 Hz, 1H).

### Example 60—Synthesis of Compound 60

### Synthetic Scheme

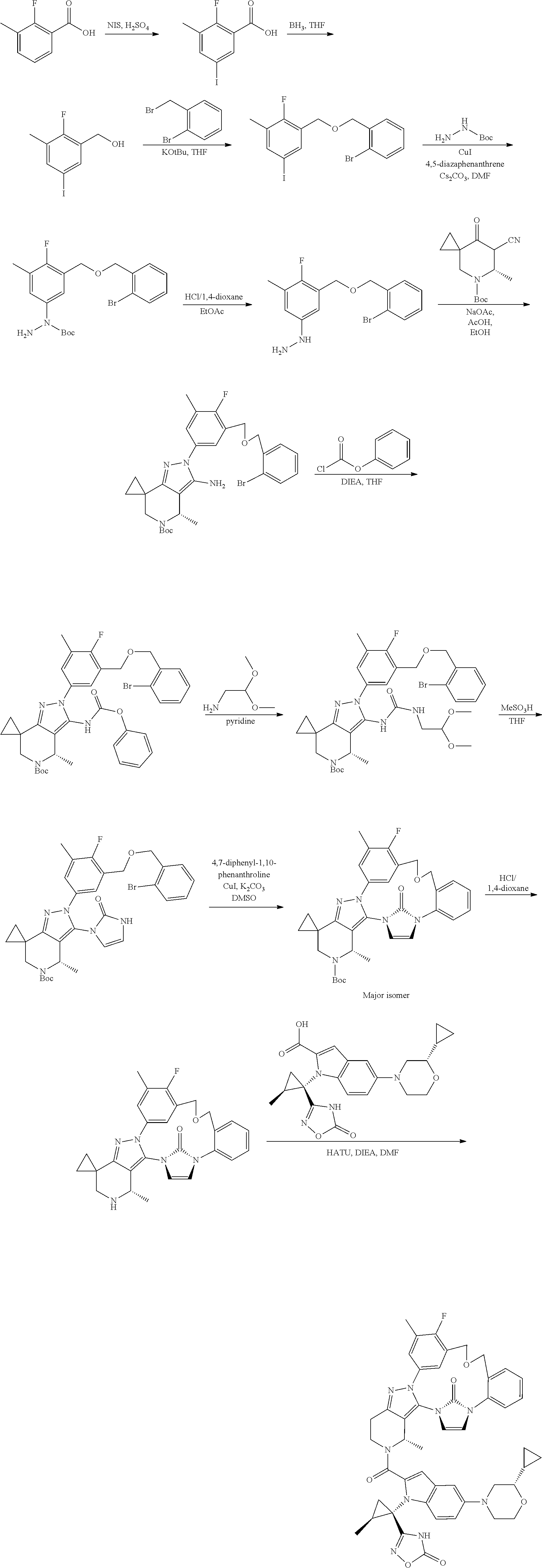

### Procedure for Synthesis of Compound 60

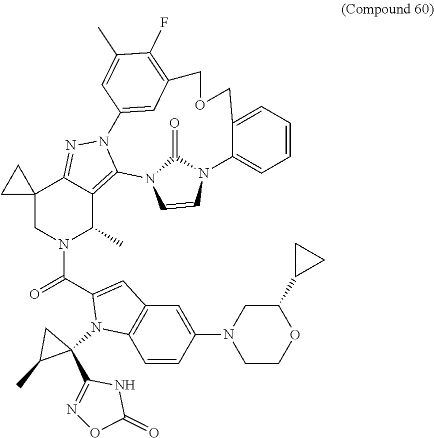

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one

Step A: To a solution of 2-fluoro-3-toluic acid (7 g, 45.4 mmol) and conc. Aq. H₂SO₄ (35 mL) under an ice bath, add 1-iodo-2,5-pyrrolidinedione (10.2 g, 45.4 mmol). After the addition, the reaction was stirred at room temperature and for 2 hours. The reaction solution was then cooled in an ice bath and diluted with water. The solution was filtered, rinsed with aq. sodium dithionate solution (~250 mg in 100 mL water), then rinsed twice with water. The filter cake was dried in a vacuum oven overnight to afford 2-fluoro-5-iodo-3-toluic acid (12 g, 42.9 mmol). m/z (ESI-pos) [M+1]=281.0

Step B: 1M Borane in THF (85 mL, 85.7 mmol) was added dropwise to a stirred solution of 2-fluoro-5-iodo-3-toluic acid (12 g, 42.9 mmol) in THF (42.9 mL) at 0° C. The resulting mixture was warmed to room temperature and stirred overnight. The mixture was poured into aq. sat. ammonium chloride solution and extracted with ethyl acetate. The combined organic phases were washed with 1M Na₂CO₃, then brine, then dried with sodium sulfate, filtered, and concentrated to afford (2-fluoro-5-iodo-3-tolyl)methanol (11 g, 41.3 mmol) which was used in the next step without purification. m/z (ESI-pos) [M-OH]=248.9

Step C: To a solution of (2-fluoro-5-iodo-3-tolyl)methanol (11.1 g, 41.7 mmol) in THF (40 mL) at 0° C. was slowly added KOtBu (6.09 g, 54.2 mmol) and stirred for 10 minutes, then o-bromo(bromomethyl)benzene (10.4 g, 41.7 mmol) was added and stirred at room temperature for 2 hours (solution turned cloudy white). The solution was then quenched with sat. NH4Cl and partitioned between Et₂O and water. The organics were dried with sodium sulfate, filtered, concentrated, and taken forward as the crude product. m/z (ESI-pos) [M+H₃O]=452.0

Step D: A solution of 3-{[(o-bromophenyl)methoxy]methyl}-2-fluoro-5-iodotoluene (16 g, 36.8 mmol), tert-butyl carbazate (6.32 g, 47.8 mmol), Cs₂CO₃ (16.8 g, 51.5 mmol), Cu (I) I (350 mg, 1.84 mmol), and 4,5-diazaphenanthrene (1.33 g, 7.36 mmol) in DMF (36.8 mL) was sparged with nitrogen for 5 minutes and heated to 80° C. for 3 hours. The solution was taken up in EtOAc (200 mL), filtered, and washed with brine (3×). The organics were dried over Na₂SO₄, filtered through celite, concentrated, and taken forward as the crude product. m/z (ESI-pos) [M+1-tBu]=383.1

Step E: To a solution of crude tert-butyl 2-(3-{[(o-bromophenyl)methoxy]methyl}-4-fluoro-5-tolyl)carbazate (15 g, 34.1 mmol) of EtOAc (45 mL) was added 45 mL of 4M HCl in dioxane and stirred at 45° C. for 12 hours, and then at room temperature overnight. The solution was filtered and rinsed with EtOAc, and the filter cake was dried in the vacuum oven to give crude product which was taken forward. m/z (ESI-pos) [M+1]=339.1

Step F: To a solution of 3-{[(o-bromophenyl)methoxy]methyl}-4-fluoro-5-toluidinoammonium chloride (1.21 g, 3.22 mmol), tert-butyl (6S)-7-cyano-6-methyl-8-oxo-5-aza-5-spiro[2.5]octanecarboxylate (850 mg, 3.22 mmol), and sodium acetate (317 mg, 3.86 mmol) in EtOH (15 mL) was added acetic acid (221 μL, 3.86 mmol) and stirred for 6 hours at 60° C., and then at room temperature overnight. The solution was then partitioned between DCM and sat. NaHCO₃, and the organics were concentrated and purified by silica gel chromatography (10-75% EtOAc/heptanes) to give tert-butyl (S)-1′-amino-2′-(3-{[(o-bromophenyl)methoxy]methyl}-4-fluoro-5-tolyl)-7′-methyl-2′,3′,6′-triaza-2′,5′,6′,7′-tetrahydrospiro[cyclopropane-1,4′-indene]-6′-carboxylate (720 mg, 1.23 mmol). m/z (ESI-pos) [M+1]=585.3

Step G: To a solution of tert-butyl (S)-1′-amino-2′-(3-{[(o-bromophenyl)methoxy]methyl}-4-fluoro-5-tolyl)-7′-methyl-2′,3′,6′-triaza-2′,5′,6′,7′-tetrahydrospiro[cyclopropane-1,4′-indene]-6′-carboxylate (715 mg, 1.22 mmol) in THF (6.11 mL) was added N-ethyldiisopropylamine (638 μL, 3.66 mmol) followed by benzenyl chloroformate (230 μL, 1.83 mmol). The reaction mixture was stirred at room temperature for 30 minutes. The reaction mixture was directly concentrated under reduced pressure to give the crude product as a yellow solid, which was used directly in the next step without any further purification. m/z (ESI-pos) [M+1]=705.4

Step H: tert-butyl (S)-2′-(3-{[(o-bromophenyl)methoxy]methyl}-4-fluoro-5-tolyl)-7′-methyl-1′-[phenyl(oxycarbonylamino)]-2′,3′,6′-triaza-2′,5′,6′,7′-tetrahydrospiro[cyclopropane-1,4′-indene]-6′-carboxylate (835 mg, 1.18 mmol) was dissolved in pyridine (2.5 mL), and then 2,2-dimethoxyethylamine (193 μL, 1.78 mmol) was added. The mixture was stirred at room temperature for 30 minutes, and then was concentrated under reduced pressure to give the crude residue as a yellow oil which was purified by column chromatography (0-5% MeOH/DCM) to give tert-butyl (S)-2′-(3-{[(o-bromophenyl)methoxy]methyl}-4-fluoro-5-tolyl)-1′-[3-(2,2-dimethoxyethyl)ureido]-7′-methyl-2′,3′,6′-triaza-2′,5′,6′,7′-tetrahydrospiro[cyclopropane-1,4′-indene]-6′-carboxylate (750 mg, 1.05 mmol). m/z (ESI-pos) [M+1]=716.4

Step I: A solution of tert-butyl (S)-2′-(3-{[(o-bromophenyl)methoxy]methyl}-4-fluoro-5-tolyl)-1′-[3-(2,2-dimethoxyethyl)ureido]-7′-methyl-2′,3′,6′-triaza-2′,5′,6′,7′-tetrahydrospiro[cyclopropane-1,4′-indene]-6′-carboxylate (720 mg, 1 mmol) and methanesulfonic acid (979 μL, 15.1 mmol) in THF (5 mL) was heated to 50° C. for 1 hour. The solution was then neutralized with sat. NaHCO₃ and stirred with Boc2O (263 mg, 1.21 mmol) for 1 hour. The solution was then partitioned between DCM and water, and the organics were concentrated and purified by silica gel chromatography (5-95% EtOAc/heptanes) to give tert-butyl (S)-2′-(3-{[(o-bromophenyl)methoxy]methyl}-4-fluoro-5-tolyl)-7′-methyl-1′-(2-oxo-2,3-dihydro-1H-imidazol-1-yl)-2′,3′,6′-triaza-2′,5′,6′,7′-tetrahydrospiro[cyclopropane-1,4′-indene]-6′-carboxylate (450 mg, 690 μmol). m/z (ESI-pos) [M+1]=652.3

Step J: A solution of tert-butyl (S)-2′-(3-{[(o-bromophenyl)methoxy]methyl}-4-fluoro-5-tolyl)-7′-methyl-1′-(2-oxo-2,3-dihydro-1H-imidazol-1-yl)-2′,3′,6′-triaza-2′,5′,6′,7′-tetrahydrospiro[cyclopropane-1,4′-indene]-6′-carboxylate (450 mg, 690 μmol), copper (I) iodide (98.5 mg, 517 μmol), bathophenanthroline (229 mg, 690 μmol), K₂CO₃ (238 mg, 1.72 mmol) was sparged with Argon for 5 minutes, and then heated to 130° C. for 2 days in a sealed vial with an Argon balloon. The solution was then diluted with Et₂O, filtered through celite, and partitioned between water and Et₂O. The organics were dried with Na₂SO₄, filtered, concentrated, and purified by silica gel chromatography (5-40% EtOAc/heptanes over 12CVs, hold at 40% for 10 CVs). The product was concentrated to give tert-butyl (8S,10M)-25-fluoro-8,26-dimethyl-29-oxospiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropane]-7-carboxylate (150 mg, 262 μmol). m/z (ESI-pos) [M+1]=572.4

Step H: A solution of tert-butyl (8S,10M)-25-fluoro-8,26-dimethyl-29-oxospiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropane]-7-carboxylate (145 mg, 254 μmol) in 1.3 mL of 4M HCl/dioxane was stirred for 1 hour at room temperature. The solution was then concentrated and dried in the vacuum oven to give crude product which was taken forward. m/z (ESI-pos) [M+1]=472.3

Step I: A solution of (8S,10M)-25-fluoro-8,26-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one-hydrogen chloride (1/1) (32 mg, 63 μmol), 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid (26.7 mg, 63 μmol), DIEA (32.9 μL, 189 μmol), and HATU (35.9 mg, 94.5 μmol) in DMF (0.3 mL) was stirred overnight at room temperature. The reaction was then purified by reverse-phase chromatography (5-95% MeCN/water, 0.1% FA), and the product was concentrated to give (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one (9 mg, 10.3 μmol). m/z (ESI-pos) [M+1]=878.7; ¹H NMR (400 MHz, chloroform-D) δ 11.33 (s, 1H), 7.97 (s, 1H), 7.60-7.37 (m, 4H), 7.22-6.95 (m, 3H), 6.73 (d, J=38.2 Hz, 1H), 6.42 (d, J=3.0 Hz, 1H), 6.27 (dd, J=23.6, 3.0 Hz, 1H), 5.91-5.82 (m, 1H), 4.79-4.61 (m, 2H), 4.32-4.18 (m, 1H), 4.14 (d, J=9.3 Hz, 1H), 4.08-4.00 (m, 2H), 3.82 (t, J=14.0 Hz, 1H), 3.69 (d, J=12.4 Hz, 1H), 3.52 (d, J=11.4 Hz, 1H), 3.35 (d, J=11.7 Hz, 1H), 3.10-2.84 (m, 2H), 2.74 (t, J=10.8 Hz, 1H), 2.35-2.28 (m, 3H), 1.77 (dd, J=51.2, 6.6 Hz, 3H), 1.47-1.28 (m, 1H), 1.17 (dd, J=18.9, 5.4 Hz, 3H), 1.07 (d, J=5.5 Hz, 1H), 0.94 (tq, J=8.3, 4.2 Hz, 1H), 0.89-0.79 (m, 1H), 0.67-0.24 (m, 4H).

### Example 61—Synthesis of Compound 61

### Synthetic Scheme

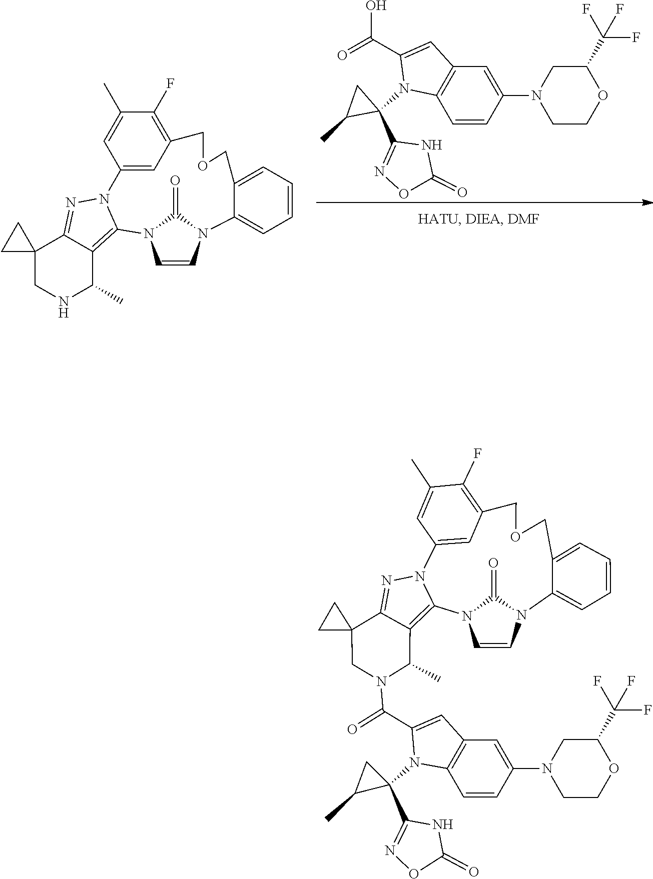

### Procedure for Synthesis of Compound 61

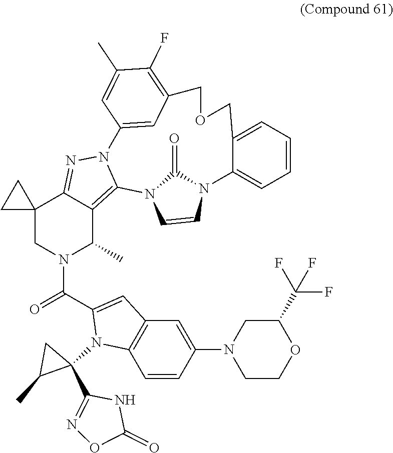

### (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)spiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one

Step A: A solution of (8S,10M)-25-fluoro-8,26-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one hydrogen chloride (40 mg, 78.7 μmol), 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid (42.7 mg, 94.5 μmol), DIEA (41.1 μL, 236 μmol), and HATU (44.9 mg, 118 μmol) in DMF (0.3 mL) was stirred overnight at room temperature. The reaction was then purified by reverse-phase chromatography (5-95% MeCN/water, 0.1% FA), and the product was concentrated to give (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)spiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one (30 mg, 33.1 μmol). m/z (ESI-pos) [M+1]=906.6; ¹H NMR (400 MHz, chloroform-D) δ 11.31 (d, J=14.2 Hz, 1H), 7.60-7.29 (m, 5H), 7.22-6.94 (m, 3H), 6.72 (d, J=31.5 Hz, 1H), 6.42 (d, J=3.0 Hz, 1H), 6.26 (dd, J=23.5, 3.0 Hz, 1H), 5.91-5.82 (m, 1H), 4.79-4.60 (m, 2H), 4.34-4.00 (m, 6H), 3.95-3.76 (m, 1H), 3.65 (d, J=14.0 Hz, 1H), 3.54 (dq, J=11.4, 2.7 Hz, 1H), 3.40-3.31 (m, 1H), 3.00-2.82 (m, 2H), 2.31 (dd, J=9.3, 1.9 Hz, 3H), 1.92-1.79 (m, 2H), 1.74-1.59 (m, 3H), 1.43-1.28 (m, 2H), 1.28-1.13 (m, 4H), 1.10-1.04 (m, 1H), 0.91-0.80 (m, 1H).

### Example 62—Synthesis of Compound 62

### Synthetic Scheme

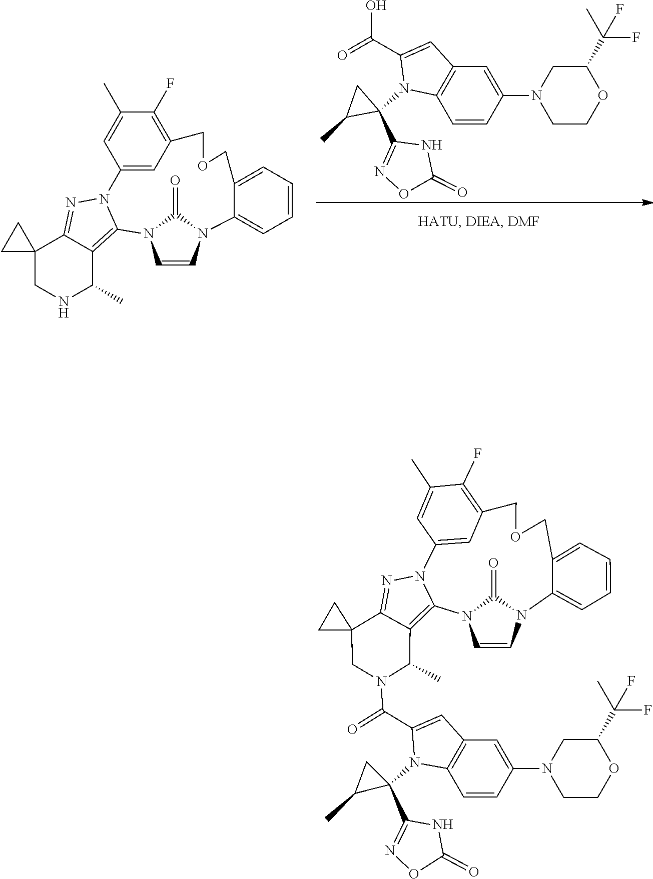

### Procedure for Synthesis of Compound 62

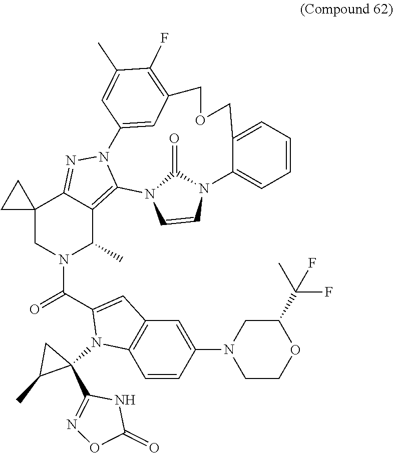

### (8S,10M)-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one

Step A: A solution of (8S,10M)-25-fluoro-8,26-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one-hydrogen chloride (1/1) (20 mg, 39.4 μmol), 5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolecarboxylic acid (21.2 mg, 47.2 μmol), DIEA (20.6 μL, 118 μmol), and HATU (22.5 mg, 59.1 μmol) in DMF (0.3 mL) was stirred overnight at room temperature. The reaction was then purified by reverse-phase chromatography (5-95% MeCN/water, 0.1% FA), and the product was concentrated to give (8S,10M)-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one (12 mg, 13.3 μmol). m/z (ESI-pos) [M+1]=902.6; ¹H NMR (400 MHz, chloroform-D) δ 11.33 (d, J=13.9 Hz, 1H), 7.59-7.32 (m, 5H), 7.21-6.93 (m, 3H), 6.71 (d, J=31.1 Hz, 1H), 6.42 (d, J=3.0 Hz, 1H), 6.26 (dd, J=23.6, 3.1 Hz, 1H), 5.91-5.81 (m, 1H), 4.79-4.60 (m, 2H), 4.33-4.00 (m, 3H), 3.92-3.76 (m, 2H), 3.65 (d, J=14.0 Hz, 1H), 3.57 (d, J=11.5 Hz, 1H), 3.36 (d, J=11.8 Hz, 1H), 2.87 (td, J=11.8, 3.3 Hz, 1H), 2.77 (t, J=11.0 Hz, 1H), 2.34-2.27 (m, 3H), 1.84-1.58 (m, 7H), 1.52 (d, J=3.6 Hz, 1H), 1.35 (dddd, J=20.4, 14.6, 10.5, 5.3 Hz, 2H), 1.17 (dd, J=20.0, 5.4 Hz, 3H), 1.05 (dd, J=14.8, 5.6 Hz, 1H), 0.83 (dt, J=11.6, 5.4 Hz, 1H).

### Example 63—Synthesis of Compound 63

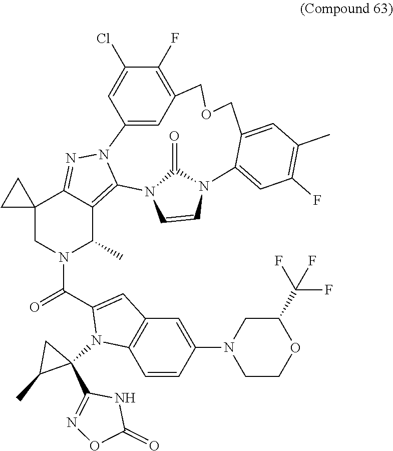

### (8S,10M)-26-chloro-17,25-difluoro-8,18-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)spiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one

Compound 63 was synthesized following the procedure of Examples 60 and 61, with (8S,10M)-25-fluoro-8,26-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one being replaced by (8S,10M)-26-chloro-17,25-difluoro-8,18-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one. m/z (ESI-pos) [M+1]=958.6; ¹H NMR (400 MHz, chloroform-D) δ 11.27 (d, J=16.8 Hz, 1H), 7.98 (s, 1H), 7.65 (ddd, J=11.8, 6.1, 2.7 Hz, 1H), 7.57 (dd, J=9.0, 6.9 Hz, 1H), 7.36 (t, J=7.6 Hz, 1H), 7.21-7.01 (m, 2H), 6.72 (dd, J=34.6, 0.9 Hz, 1H), 6.43 (d, J=3.1 Hz, 1H), 6.28 (dd, J=31.4, 3.0 Hz, 1H), 5.93-5.81 (m, 1H), 4.80-4.60 (m, 2H), 4.25 (d, J=9.5 Hz, 1H), 4.21-3.99 (m, 5H), 3.90 (td, J=11.4, 2.6 Hz, 1H), 3.72 (dd, J=52.3, 13.8 Hz, 1H), 3.54 (d, J=11.5 Hz, 1H), 3.36 (d, J=12.3 Hz, 1H), 3.00-2.82 (m, 2H), 2.32 (dd, J=9.2, 1.7 Hz, 3H), 1.75 (dd, J=50.9, 6.7 Hz, 3H), 1.66-1.30 (m, 5H), 1.22-1.12 (m, 3H), 0.90-0.81 (m, 1H).

### Example 64—Synthesis of Compound 64

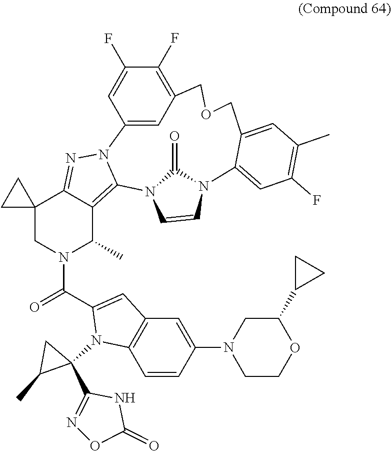

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25,26-trifluoro-8,18-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one

Compound 64 was synthesized following the procedure of Example 60, with (8S,10M)-25-fluoro-8,26-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one being replaced by (8S,10M)-17,25,26-trifluoro-8,18-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one. m/z (ESI-pos) [M+1]=914.6; ¹H NMR (400 MHz, chloroform-D) δ 11.28 (d, J=16.4 Hz, 1H), 7.98 (s, 1H), 7.59-7.51 (m, 1H), 7.50-7.39 (m, 1H), 7.35 (dd, J=9.2, 6.4 Hz, 1H), 7.17-7.03 (m, 1H), 6.91 (d, J=34.3 Hz, 1H), 6.77-6.62 (m, 1H), 6.44 (dd, J=8.4, 3.1 Hz, 1H), 6.30 (dd, J=30.7, 3.1 Hz, 1H), 5.93-5.81 (m, 1H), 4.80-4.62 (m, 3H), 4.32-4.17 (m, 2H), 4.10-3.98 (m, 3H), 3.85-3.74 (m, 1H), 3.67 (d, J=14.0 Hz, 1H), 3.52 (d, J=11.3 Hz, 1H), 3.34 (d, J=11.7 Hz, 1H), 2.99-2.82 (m, 2H), 2.72 (t, J=10.9 Hz, 1H), 2.35-2.28 (m, 3H), 1.68 (d, J=6.7 Hz, 3H), 1.53-1.49 (m, 1H), 1.41-1.31 (m, 2H), 1.21-1.13 (m, 3H), 0.95 (tq, J=8.6, 4.3 Hz, 1H), 0.66-0.52 (m, 2H), 0.46 (dq, J=9.2, 4.7 Hz, 1H), 0.30 (dt, J=9.5, 4.6 Hz, 1H).

### Example 65—Synthesis of Compound 65

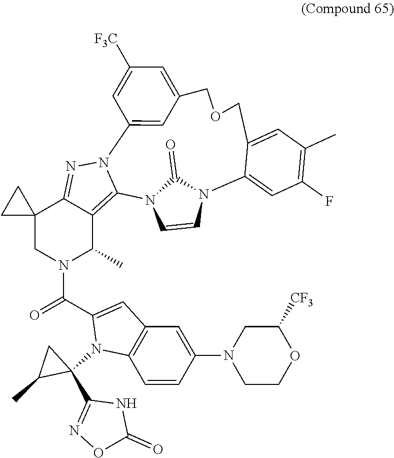

### (8S,10M)-17-fluoro-8,18-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-26-(trifluoromethyl)spiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one

Compound 65 was synthesized following the procedure of Example 60 and 61, with (8S,10M)-26-chloro-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one being replaced by (8S,10M)-17-fluoro-8,18-dimethyl-26-(trifluoromethyl)spiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one. m/z (ESI-pos) [M+1]=974.6; ¹H NMR (400 MHz, chloroform-D) δ 11.26 (d, J=18.7 Hz, 1H), 7.88 (d, J=17.1 Hz, 1H), 7.57 (t, J=8.7 Hz, 1H), 7.39 (d, J=9.3 Hz, 1H), 7.34 (d, J=7.7 Hz, 2H), 7.18-7.05 (m, 2H), 6.73 (dd, J=32.8, 0.8 Hz, 1H), 6.47 (d, J=3.1 Hz, 1H), 6.31 (dd, J=42.0, 3.1 Hz, 1H), 5.94-5.83 (m, 1H), 4.65 (dd, J=25.1, 11.4 Hz, 2H), 4.30-4.24 (m, 1H), 4.23-4.09 (m, 3H), 4.08-3.98 (m, 2H), 3.90 (td, J=11.5, 2.6 Hz, 1H), 3.74 (dd, J=50.4, 13.8 Hz, 1H), 3.54 (d, J=11.4 Hz, 1H), 3.36 (d, J=11.9 Hz, 1H), 3.00-2.82 (m, 2H), 2.31 (dd, J=8.6, 1.8 Hz, 3H), 1.76 (dd, J=53.5, 6.7 Hz, 3H), 1.48-1.28 (m, 2H), 1.27-1.13 (m, 3H), 0.86 (dt, J=9.9, 6.0 Hz, 1H).

### Example 66—Synthesis of Compound 66

### Synthetic Scheme

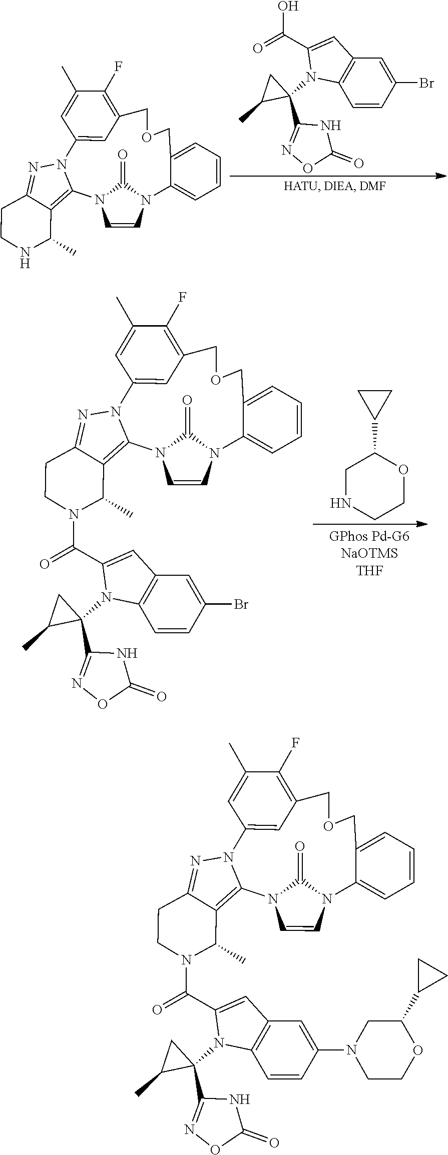

### Procedure for Synthesis of Compound 66

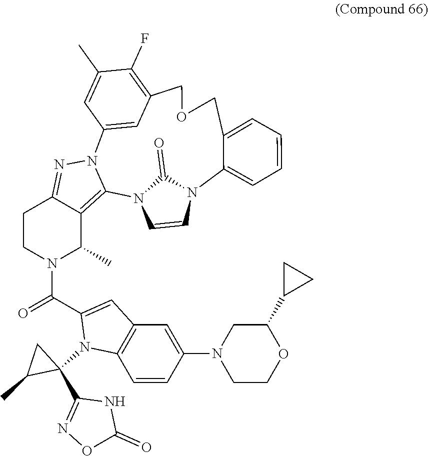

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20), 16,18,24(28),25-nonaen-29-one

Step A: To a solution of (8S,10M)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (0.5 g, 1.04 mmol) and 5-bromo-1-((1S,2S)-2-methyl-1-(5-oxo-4,5-dihydro-1,2,4-oxadiazol-3-yl)cyclopropyl)-1H-indole-2-carboxylic acid (432 mg, 1.14 mmol) in DMF (3.46 mL) was added DIEA (723 μL, 4.15 mmol) and HATU (552 mg, 1.45 mmol), and the mixture was stirred at room temperature overnight. The solution was then taken up in EtOAc and washed with water (3×). The organics were concentrated and purified by silica gel chromatography (10-95% EtOAc/heptanes) to give (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (710 mg, 881 μmol). m/z (ESI-pos) [M+1]=805.4

Step B: A 4 mL vial was charged with (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (20 mg, 25.9 μmol), (S)-2-cyclopropylmorpholine (3.16 mg, 24.8 μmol), GPhos Pd-G6 (2.44 mg, 2.59 μmol), and sodium trimethylsilanolate (11.6 mg, 103 μmol), in THF (646 μL). The mixture was sparged for 5 minutes with Argon and then heated to 70° C. overnight. The next morning the solution was filtered and purified by reverse-phase chromatography (5-95% MeCN/water, 0.1% FA). The product was concentrated to give (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (18 mg, 21.1 μmol). m/z (ESI-pos) [M+1]=852.7; ¹H NMR (400 MHz, chloroform-D) δ 11.33 (s, 1H), 7.58-7.42 (m, 6H), 7.12 (ddt, J=11.9, 5.6, 2.5 Hz, 2H), 6.76-6.63 (m, 1H), 6.44 (d, J=3.0 Hz, 1H), 6.27 (dd, J=13.8, 3.0 Hz, 1H), 5.79 (q, J=6.6 Hz, 1H), 4.80-4.62 (m, 3H), 4.48 (dd, J=13.8, 5.0 Hz, 1H), 4.40-4.27 (m, 1H), 4.19-4.11 (m, 1H), 4.09-4.01 (m, 1H), 3.79 (td, J=11.4, 2.6 Hz, 1H), 3.62 (td, J=13.1, 4.1 Hz, 1H), 3.52 (d, J=11.6 Hz, 1H), 3.34 (d, J=11.8 Hz, 1H), 3.13 (ddd, J=17.4, 12.4, 5.3 Hz, 1H), 3.07-3.00 (m, 1H), 2.98-2.90 (m, 1H), 2.85 (td, J=11.6, 3.2 Hz, 1H), 2.71 (dd, J=11.6, 10.1 Hz, 1H), 2.36-2.28 (m, 3H), 1.94-1.75 (m, 2H), 1.65 (d, J=6.7 Hz, 3H), 1.59-1.47 (m, 2H), 1.24-1.10 (m, 3H), 0.96 (qt, J=8.6, 4.8 Hz, 1H), 0.67-0.40 (m, 3H), 0.29 (dq, J=9.6, 4.8 Hz, 1H)

### Example 67—Synthesis of Compound 67

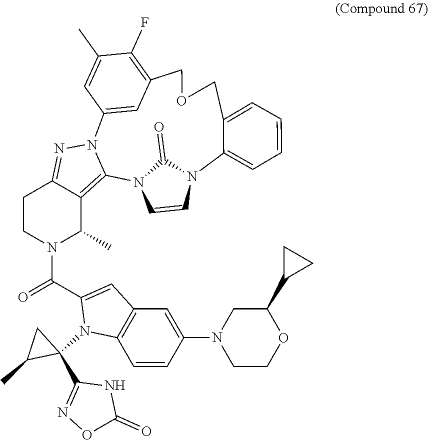

### (8S,10M)-7-({5-[(R)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20), 16,18,24(28),25-nonaen-29-one

Compound 67 was synthesized following the procedure of Example 66, with (R)-2-cyclopropylmorpholine being replaced by(S)-2-cyclopropylmorpholine. m/z (ESI-pos) [M+1]=852.7; ¹H NMR (400 MHz, chloroform-D) δ 11.32 (s, 1H), 7.58-7.50 (m, 2H), 7.50-7.42 (m, 4H), 7.18-7.09 (m, 2H), 6.79-6.62 (m, 1H), 6.44 (dd, J=3.2, 1.4 Hz, 1H), 6.28 (dd, J=3.1, 1.4 Hz, 1H), 5.79 (q, J=6.5 Hz, 1H), 4.81-4.62 (m, 3H), 4.51-4.43 (m, 1H), 4.40-4.27 (m, 1H), 4.15 (d, J=9.4 Hz, 1H), 4.04 (d, J=11.2 Hz, 1H), 3.87-3.73 (m, 1H), 3.62 (t, J=12.1 Hz, 1H), 3.52 (d, J=11.5 Hz, 1H), 3.33 (d, J=11.6 Hz, 1H), 3.18-3.06 (m, 1H), 3.02 (d, J=15.0 Hz, 1H), 2.93 (t, J=9.3 Hz, 1H), 2.85 (t, J=11.3 Hz, 1H), 2.69 (t, J=10.9 Hz, 1H), 2.32 (d, J=9.4 Hz, 3H), 1.94-1.82 (m, 1H), 1.79-1.62 (m, 3H), 1.61-1.47 (m, 1H), 1.27-1.17 (m, 3H), 1.10 (dd, J=13.8, 6.9 Hz, 1H), 0.95 (dt, J=8.8, 4.8 Hz, 1H), 0.58 (ddt, J=17.3, 13.2, 6.6 Hz, 2H), 0.45 (dd, J=9.4, 4.8 Hz, 1H), 0.29 (dd, J=9.5, 4.8 Hz, 1H).

### Example 68—Synthesis of Compound 68

### Synthetic Scheme

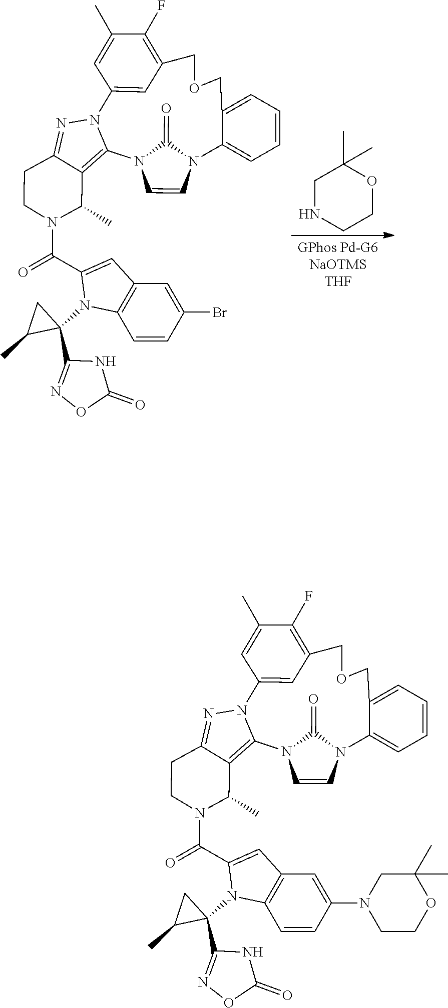

### Procedure for Synthesis of Compound 68

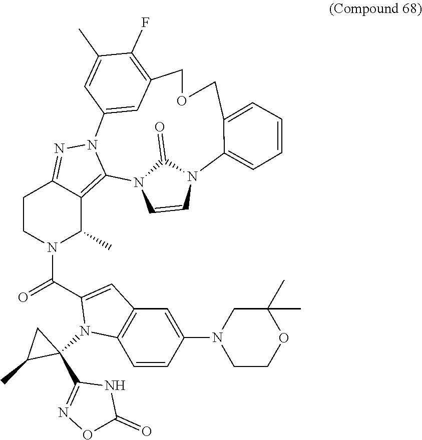

### (8S,10M)-7-{[5-(2,2-dimethyl-4-morpholinyl)-1-[(1S,2S)-2-methyl-1-(5-oxo-4,5-dihydro-1,2,4-oxadiazol-3-yl)cyclopropyl]-2-indolyl]carbonyl}-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20), 16,18,24(28),25-nonaen-29-one

Step A: A 4 mL vial was charged with (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (20 mg, 25.9 μmol), 2,2-dimethylmorpholine (7.15 mg, 62.1 μmol), GPhos Pd-G6 (2.44 mg, 2.59 μmol), sodium trimethylsilanolate (11.6 mg, 103 μmol), followed by addition of THF (646 μL). The mixture was sparged for 5 minutes with Argon and then heated to 70° C. overnight. The solution was then filtered and purified by reverse-phase chromatography (5-95% MeCN/water, 0.1% FA). The product was concentrated to give (8S,10M)-7-{[5-(2,2-dimethyl-4-morpholinyl)-1-[(1S,2S)-2-methyl-1-(5-oxo-4,5-dihydro-1,2,4-oxadiazol-3-yl)cyclopropyl]-2-indolyl]carbonyl}-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20), 16,18,24(28),25-nonaen-29-one (16 mg, 18.3 μmol). m/z (ESI-pos) [M+1]=902.6; ¹H NMR (400 MHz, chloroform-D) δ 7.58-7.40 (m, 5H), 7.13-7.06 (m, 3H), 6.63 (s, 1H), 6.44 (d, J=3.0 Hz, 1H), 6.26 (dd, J=13.8, 3.1 Hz, 1H), 5.89-5.74 (m, 1H), 4.80-4.62 (m, 2H), 4.46 (dd, J=13.8, 5.1 Hz, 1H), 4.40-4.27 (m, 1H), 4.19-4.11 (m, 1H), 3.95-3.88 (m, 2H), 3.62 (td, J=13.2, 4.2 Hz, 1H), 3.19-2.98 (m, 5H), 2.90 (s, 2H), 2.36-2.29 (m, 3H), 1.94-1.75 (m, 2H), 1.65 (d, J=6.7 Hz, 3H), 1.59-1.46 (m, 2H), 1.35 (s, 7H), 1.19 (d, J=5.9 Hz, 3H).

### Example 69 and 70—Synthesis of Compound 69 and 70

### Synthetic Scheme

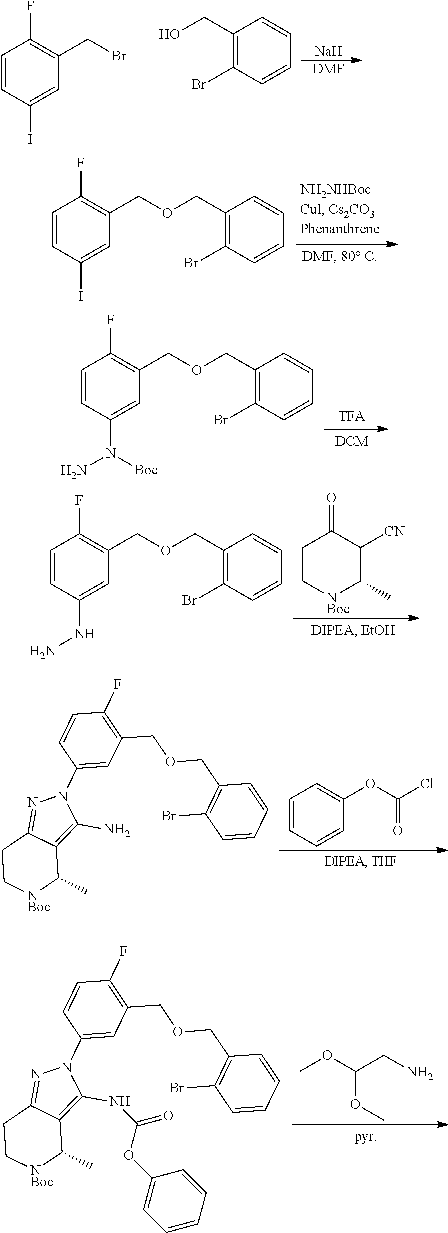

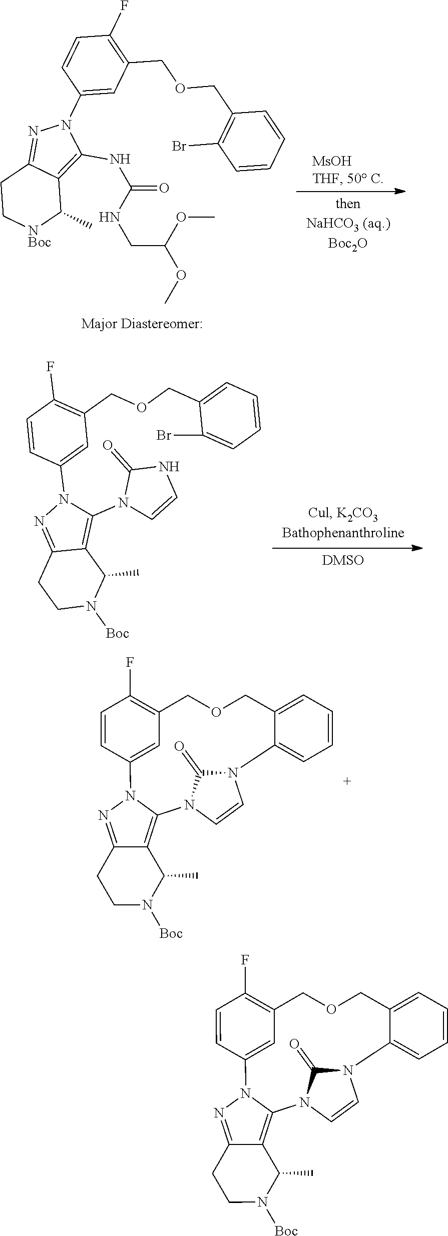

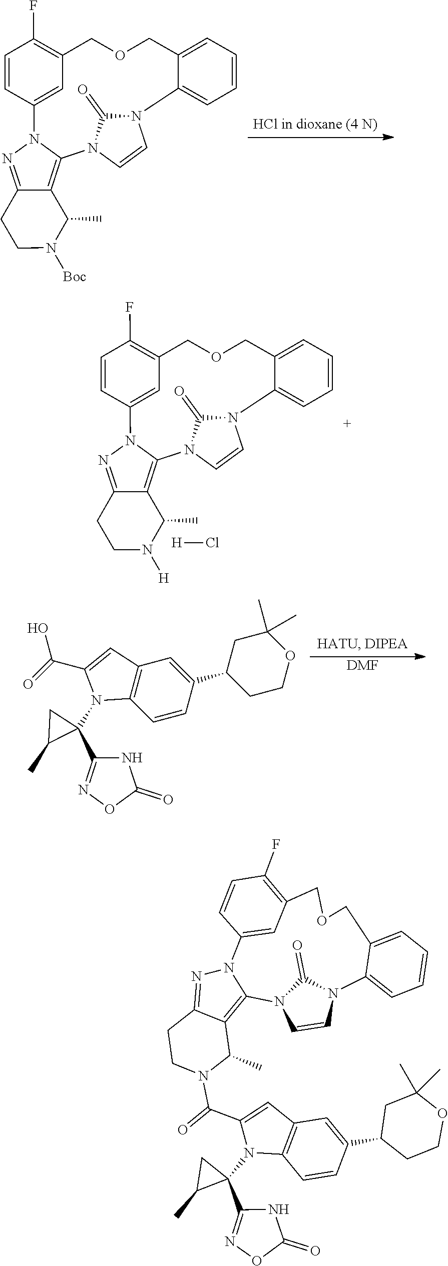

### Procedure for Synthesis of Compound 69

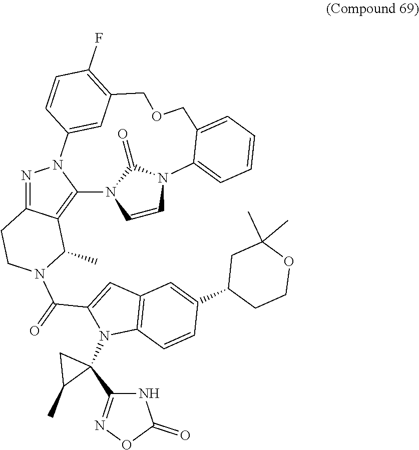

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To the solution of (o-bromophenyl) methanol (1.29 g, 6.9 mmol) in DMF (23 mL) at 0° C., sodium hydride 60% w/w (414 mg, 10.3 mmol) was added. Stir 20 min at 0° C. then 2-(bromomethyl)-1-fluoro-4-iodobenzene (2.5 g, 7.93 mmol) was added portionwise. The resulting mixture was allowed to stir at 0° C. for 20 min then warmed to room temperature for an additional 20 min. The reaction mixture was quenched at 0° C. with sat. aq. NH₄Cl (20 mL), diluted with ethyl acetate (50 mL), washed with water (2×50 mL) and the aqueous layer was extracted with ethyl acetate (2×50 mL). The combined organic layers were washed with brine (2×50 mL), dried over Na₂SO₄ and concentrated in vacuo. The crude product was purified by column chromatography (0-10% EtOAc in heptane) to give 2-bromo-4,5-difluoro-1 2-{[(o-bromophenyl)methoxy]methyl}-1-fluoro-4-iodobenzene (2.69 g, 6.39 mmol) as a colorless oil. m/z (ESI-pos) M+1=not found-does not ionize

Step B: A solution of 2-{[(o-bromophenyl)methoxy]methyl}-1-fluoro-4-iodobenzene (2.69 g, 6.39 mmol), tert-butyl carbazate (1.1 g, 8.31 mmol), dicaesium carbonate (2.91 g, 8.94 mmol), CuI (60.8 mg, 319 μmol), and 4,5-diazaphenanthrene (230 mg, 1.28 mmol) in DMF (6.4 mL) was sparged with Ar for 5 min and heated to 80° C. for 3 h. The solution was cooled to room temperature, diluted with EtOAc (20 mL), and washed with brine (3×20 mL). The organics were dried over Na₂SO₄, filtered, and concentrated. The crude product purified by column chromatography (5-30% EtOAc in heptane) to give tert-butyl 2-(3-{[(o-bromophenyl)methoxy]methyl}-4-fluorophenyl)carbazate (2.45 g, 5.76 mmol). m/z (ESI-pos) M+1-tert-butyl=369.1/371.1

Step C: To a solution of tert-butyl 2-(3-{[(o-bromophenyl)methoxy]methyl}-4-fluorophenyl) carbazate (2.45 g, 5.76 mmol) in DCM (19 mL), trifluoroacetic acid (4.41 mL, 57.6 mmol) was added. The reaction stirred at room temperature for 2.5 h. Upon completion, the reaction was *carefully* quenched with sat. aq. Na₂CO₃ (~30 mL), extracted DCM (1×50 mL), then washed with water (2×50 mL) and brine (1×50 mL). The combined aqueous phases were extracted with with 20% IPA/CHCl₃ (2×50 mL) and the combined organics were washed with sat. aq. NaHCO₃ (3×50 mL) and then dried over Na₂SO₄, filtered, and concentrated in vacuo to provide (3-{[(o-bromophenyl)methoxy]methyl}-4-fluorophenyl)hydrazine (1.75 g, 5.38 mmol) as a yellow oil which was used without purification in the following step. m/z (ESI-pos) M+1=325.1/327.1

Step D: To a solution of crude (3-{[(o-bromophenyl)methoxy]methyl}-4-fluorophenyl)hydrazine (1.75 g, 5.38 mmol) in EtOH (21.5 mL) was added N-ethyldiisopropylamine (1.41 mL, 8.07 mmol) then tert-butyl(2S)-3-cyano-2-methyl-4-oxo-1-piperidinecarboxylate (1.54 g, 6.46 mmol). The mixture was stirred at 80° C. for 20 h. After completion, the reaction mixture was cooled to ambient temperature and the volatiles were evaporated under reduced pressure to give the crude compound which was purified by column chromatography (15-60% EtOAc in heptane) to give tert-butyl (S)-3-amino-2-(3-{[(o-bromophenyl)methoxy]methyl}-4-fluorophenyl)-4-methyl-1,2,5-triaza-2,4,6,7-tetrahydro-5H-indene-5-carboxylate (1.7 g, 3.12 mmol) as an off-white solid over 2 steps. m/z (ESI-pos) M+1=545.3/547.3

Step E: To a solution of tert-butyl (S)-3-amino-2-(3-{[(o-bromophenyl)methoxy]methyl}-4-fluorophenyl)-4-methyl-1,2,5-triaza-2,4,6,7-tetrahydro-5H-indene-5-carboxylate (1.7 g, 3.12 mmol) in THF (15.6 mL) was added N-ethyldiisopropylamine (1.63 mL, 9.35 mmol) followed by benzenyl chloroformate (782 μL, 6.23 mmol). The reaction mixture was stirred at 25° C. for 30 min. The reaction mixture was directly concentrated under reduced pressure to give the crude tert-butyl (S)-2-(3-{[(o-bromophenyl)methoxy]methyl}-4-fluorophenyl)-4-methyl-3-[phenyl(oxycarbonylamino)]-1,2,5-triaza-2,4,6,7-tetrahydro-5H-indene-5-carboxylate (2.07 g, 3.11 mmol; assumed quant. yield) as a yellow oil, which was used directly in the next step without any further purification. m/z (ESI-pos) M+1=665.3/667.3

Step F: Crude tert-butyl (S)-2-(3-{[(o-bromophenyl)methoxy]methyl}-4-fluorophenyl)-4-methyl-3-[phenyl(oxycarbonylamino)]-1,2,5-triaza-2,4,6,7-tetrahydro-5H-indene-5-carboxylate (2.07 g, 3.11 mmol) was dissolved in pyridine (10.4 mL) and then 2,2-dimethoxyethylamine (508 μL, 4.67 mmol) was added. The mixture was stirred at room temperature for 30 min and then directly concentrated under reduced pressure. The crude residue was purified by column chromatography (5-80% EtOAc in heptane) to give tert-butyl (S)-2-(3-{[(o-bromophenyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (1.48 g, 2.19 mmol) as a white solid over 2 steps. m/z (ESI-pos) M+1=676.4/678.3

Step G: To a solution of tert-butyl (S)-2-(3-{[(o-bromophenyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (1.48 g, 2.19 mmol) in THF (10.9 mL) was added methanesulfonic acid (426 μL, 6.56 mmol). The mixture was stirred at 55° C. for 1 h and then cooled to ambient temperature. Saturated aqueous NaHCO₃ was added until pH 8 then tert-butyl-tert-butyl (oxycarbonyloxy) formylate (525 mg, 2.41 mmol) was added, and the resulting mixture was stirred at room temperature for 30 min. The reaction mixture was diluted with water (20 mL) and was extracted with ethyl acetate (30 mL×2). The combined organic layers were washed with brine (30 mL) and dried over anhydrous Na₂SO₄, filtered and concentrated under reduced pressure. The crude compound, which was purified by column chromatography (10-90% EtOAc in heptane) to give tert-butyl (S)-2-(3-{[(0-bromophenyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (1.28 g, 2.09 mmol) as an off-white solid. m/z (ESI-pos) M+1=612.4/614.3

Step H: A solution of tert-butyl (S)-2-(3-{[(o-bromophenyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (0.4 g, 653 μmol), copper iodide (93.3 mg, 490 μmol), bathophenanthroline (217 mg, 653 μmol), and dipotassium carbonate (226 mg, 1.63 mmol) in DMSO (8.2 mL) was sparged with Ar for 5 minutes, and then heated to 130° C. in a sealed vial for 89 h. The reaction was cooled to ambient temperature and diluted with EtOAc and sat. aq. NH₄Cl, the workup solution (organic and aqueous) was filtered through a pad of celite. The filtrate was partitioned and the organic phase was washed with brine, dried with sodium sulfate, filtered, concentrated. The crude was purified by silica gel chromatography (5-50% EtOAc in heptane) to give two diastereomers, whose stereochemistry was arbitrarily assigned, tert-butyl (8S,10M)-25-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (99 mg, 186 μmol) and tert-butyl (8S,10P)-25-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (27 mg, 347 μmol). m/z (ESI-pos) M+1=532.3

Step I: Hydrogen chloride in dioxane (4N, 1.5 mL) was added to a vial containing tert-butyl (8S,10M)-25-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (19 mg, 35.7 μmol) and the solution stirred at room temperature for 20 min. Upon completion, the solvent was evaporated to give crude (8S,10M)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (16 mg; 35.7 μmol) which was used in the next step without purification. m/z (ESI-pos) M+1=432.3

Step J: To a solution of (8S,10M)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (16 mg; 35.7 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (18.9 mg, 45.8 μmol) in DMF (700 μL) was added added N-ethyldiisopropylamine (31 μL, 220 μmol) and HATU (26.8 mg, 70.5 μmol). The reaction stirred at room temperature overnight. The reaction mixture was purified by Prep HPLC (Isco ACCQ Prep, Gemini column NX—C18, 250×21 mm, 20 ml/min, 45 minute separation, 20 to 80% H₂O/ACN with 0.1% Formic acid buffer), pure fractions were collected and lyophilized to give (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (19.5 mg, 23.6 μmol). m/z (ESI-pos) M+1=825.5; ¹H NMR (400 MHz, CHLOROFORM-D) δ 11.28 (d, J=18.9 Hz, 1H), 7.65-7.41 (m, 7H), 7.38-7.26 (m, 2H), 7.18 (t, J=9.0 Hz, 1H), 6.74 (d, J=33.3 Hz, 1H), 6.48-5.85 (m, 2H), 5.80 (q, J=6.6 Hz, 1H), 5.29-4.40 (m, 3H), 4.35-4.12 (m, 2H), 3.84 (d, J=15.0 Hz, 2H), 3.70-3.33 (m, 1H), 3.20-2.93 (m, 3H), 1.96-1.54 (m, 10H), 1.33 (s, 3H), 1.27 (s, 3H), 1.24-1.08 (m, 4H).

### Procedure for Synthesis of Compound 70

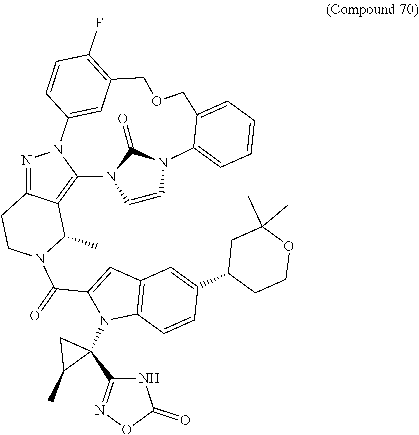

### (8S,10P)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 70 was synthesized following the procedure of Example 69 with tert-butyl (8S,10M)-25-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate being replaced with tert-butyl (8S,10P)-25-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate in step I. m/z (ESI-pos) M+1=825.5; ¹H NMR (400 MHz, CHLOROFORM-D) δ 11.28 (d, J=27.2 Hz, 1H), 7.66-7.26 (m, 8H), 7.24-7.11 (m, 2H), 6.70 (s, 1H), 6.39 (dd, J=17.8, 3.0 Hz, 1H), 6.18 (dd, J=24.6, 3.1 Hz, 1H), 5.92-5.22 (m, 1H), 4.90-4.63 (m, 2H), 4.47 (dd, J=13.8, 5.1 Hz, 1H), 4.28-4.02 (m, 2H), 3.85 (dq, J=13.9, 4.2 Hz, 2H), 3.48 (dtd, J=80.6, 13.2, 4.5 Hz, 1H), 3.23-2.94 (m, 3H), 1.90 (t, J=5.5 Hz, 1H), 1.81-1.47 (m, 8H), 1.45-1.31 (m, 4H), 1.24 (dd, J=30.1, 4.1 Hz, 5H), 0.99 (d, J=6.1 Hz, 1H).

### Example 71—Synthesis of Compound 71

### Synthetic Scheme

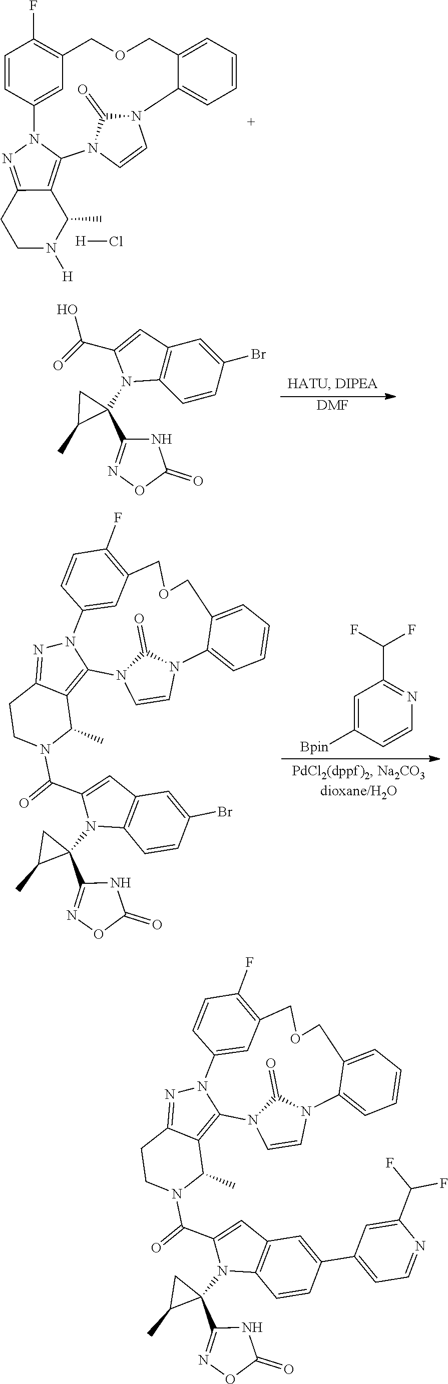

### Procedure for Synthesis of Compound 71

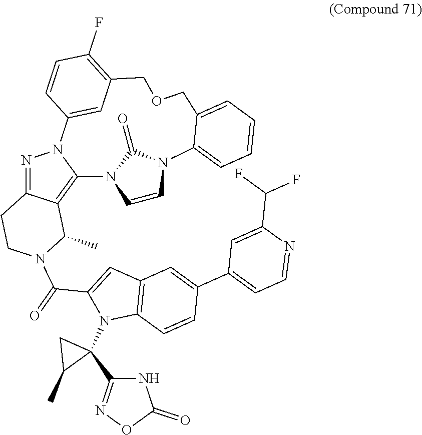

### (8S,10M)-7-({5-[2-(difluoromethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (8S,10M)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (67.8 mg; 145 μmol) in DMF (2.9 mL) was added N-ethyldiisopropylamine (126 μL, 724 μmol), 5-bromo-1-[(1S,2S)-2-methyl-1-(5-oxo-4,5-dihydro-1,2,4-oxadiazol-3-yl)cyclopropyl]-2-indolecarboxylic acid (65.8 mg, 174 μmol), and HATU (110 mg, 290 μmol). The mixture stirred at room temperature overnight. The product was purified by Prep HPLC (Isco ACCQ Prep, Gemini column NX—C18, 250×21 mm, 20 ml/min, 45 minute separation, 20 to 80% H₂O/ACN with 0.1% Formic acid buffer), pure fractions were collected and concentrated to give (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (94 mg, 119 μmol). m/z (ESI-pos) M+1=791.4/793.4

Step B: A solution of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (7.2 mg, 9.1 μmol), PdCl₂(dppf) (0.7 mg, 0.91 μmol), 2-[2-(difluoromethyl)-4-pyridyl]-4,4,5,5-tetramethyl-1,3,2-dioxaborolane (4.64 mg, 18.2 μmol) and disodium carbonate (4.82 mg, 45.5 μmol) in 1,4-dioxane (300 μL) and H₂O (100 μL) was heated to 80° C. for 1 h. The mixture was cooled to room temperature and filtered through an HPLC filter, rinsing with MeOH/MeCN then purified directly by Prep HPLC (Isco ACCQ Prep, Gemini column NX—C18, 250×21 mm, 20 ml/min, 45 minute separation, 20 to 70% H₂O/ACN with 0.1% Formic acid buffer), pure fractions were collected and lyophilized to give (8S,10M)-7-({5-[2-(difluoromethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (2.63 mg, 3.13 μmol). m/z (ESI-pos) M+1=840.5; ¹H NMR (400 MHz, CHLOROFORM-D) δ 11.26 (s, 1H), 8.69 (d, J=5.2 Hz, 1H), 7.94 (d, J=41.5 Hz, 2H), 7.78 (d, J=8.6 Hz, 1H), 7.70-7.42 (m, 6H), 7.31 (s, 1H), 7.18 (t, J=9.0 Hz, 1H), 7.01-6.54 (m, 2H), 6.44-5.84 (m, 2H), 5.86-5.76 (m, 1H), 4.83-4.68 (m, 2H), 4.47 (d, J=10.1 Hz, 1H), 4.24 (dd, J=61.4, 9.2 Hz, 2H), 3.68 (t, J=12.9 Hz, 1H), 3.20-2.99 (m, 2H), 1.98 (t, J=6.0 Hz, 1H), 1.76-1.24 (m, 9H).

### Example 72—Synthesis of Compound 72

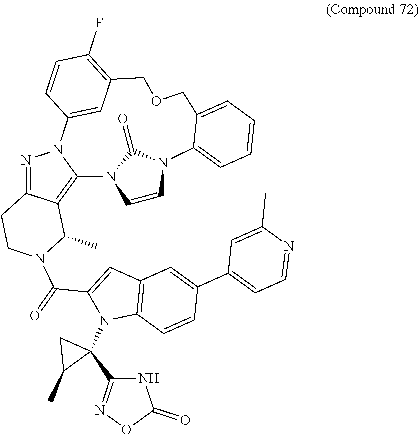

### (8S,10M)-25-fluoro-8-methyl-7-({1-[(1S,2S)-2-methyl-1-(5-oxo-4,5-dihydro-1,2,4-oxadiazol-3-yl)cyclopropyl]-5-(2-methyl-4-pyridyl)-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 72 was synthesized following the procedure of Example 71, with 2-[2-(difluoromethyl)-4-pyridyl]-4,4,5,5-tetramethyl-1,3,2-dioxaborolane being replaced with (2-methyl-4-pyridyl) boranediol in step B. m/z (ESI-pos) M+1=804.5; ¹H NMR (400 MHz, CHLOROFORM-D) δ 11.28 (s, 1H), 8.54 (d, J=5.2 Hz, 1H), 7.99-7.91 (m, 1H), 7.74 (d, J=8.6 Hz, 1H), 7.67-7.38 (m, 6H), 7.33 (ddd, J=14.0, 5.6, 2.2 Hz, 2H), 7.18 (t, J=9.0 Hz, 1H), 6.85 (d, J=34.2 Hz, 1H), 6.48-5.87 (m, 2H), 5.83 (q, J=6.7 Hz, 1H), 4.99-4.64 (m, 2H), 4.48 (dd, J=13.8, 5.1 Hz, 1H), 4.36-4.14 (m, 2H), 3.75-3.38 (m, 1H), 3.23-2.99 (m, 2H), 2.63 (s, 3H) 2.01-1.80 (m, 3H), 1.69 (d, J=6.7 Hz, 3H), 1.23-1.15 (m, 4H).

### Example 73—Synthesis of Compound 73

### Synthetic Scheme

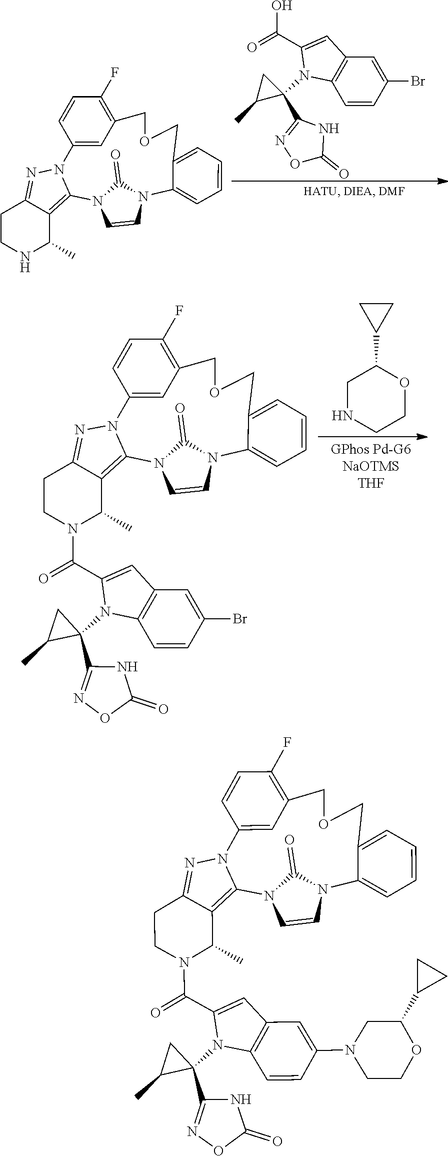

### Procedure for Synthesis of Compound 73

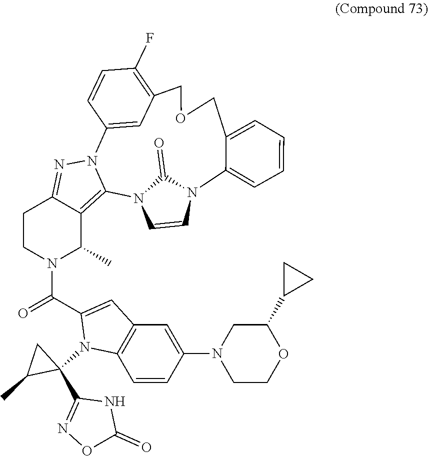

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20), 16,18,24(28),25-nonaen-29-one

Step A: A solution of (8S,10M)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (0.2 g, 427 μmol), 5-bromo-1-[(1S,2S)-2-methyl-1-(5-oxo-4,5-dihydro-1,2,4-oxadiazol-3-yl)cyclopropyl]-2-indolecarboxylic acid (194 mg, 513 μmol), DIEA (223 μL, 1.28 mmol), and DMF (2.14 mL) was stirred overnight at room temperature. The solution was then taken up in EtOAc and washed with water (3×). The organics were concentrated and purified by silica gel chromatography (10-95% EtOAc/heptanes) to give (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (232 mg, 293 μmol). m/z (ESI-pos) [M+1]=791.3

Step B: A 4 mL vial was charged with (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (20 mg, 25.9 μmol), (S)-2-cyclopropylmorpholine (7.71 mg, 60.6 μmol), GPhos Pd-G6 (2.44 mg, 2.59 μmol), sodium trimethylsilanolate (11.6 mg, 103 μmol), followed by addition of THF (646 μL). The mixture was sparged for 5 minutes with Argon and then heated to 70° C. overnight. The next morning the solution was filtered and purified by reverse-phase chromatography (5-95% MeCN/water, 0.1% FA). The product was then concentrated and repurified by silica gel chromatography (5-95% EtOAc/heptanes), and the product was concentrated to give (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (4 mg, 4.77 μmol). m/z (ESI-pos) [M+1]=902.6; ¹H NMR (400 MHz, chloroform-D) δ 11.32 (s, 1H), 7.66-7.58 (m, 1H), 7.57-7.42 (m, 4H), 7.39-7.27 (m, 1H), 7.22-7.10 (m, 3H), 6.65 (d, J=0.9 Hz, 1H), 6.43 (d, J=3.0 Hz, 1H), 6.30-6.22 (m, 1H), 5.79 (q, J=6.6 Hz, 1H), 4.82-4.68 (m, 2H), 4.48 (dd, J=13.8, 5.1 Hz, 1H), 4.29 (dd, J=25.5, 9.2 Hz, 1H), 4.19-4.07 (m, 1H), 4.07-4.02 (m, 1H), 3.78 (td, J=11.4, 2.6 Hz, 1H), 3.63 (td, J=13.1, 4.1 Hz, 1H), 3.51 (d, J=11.5 Hz, 1H), 3.34 (d, J=11.7 Hz, 1H), 3.20-3.07 (m, 1H), 3.06-3.00 (m, 1H), 2.96-2.89 (m, 1H), 2.85 (td, J=11.6, 3.3 Hz, 1H), 2.75-2.65 (m, 1H), 1.89 (t, J=6.1 Hz, 1H), 1.66 (d, J=6.7 Hz, 3H), 1.20 (d, J=5.9 Hz, 3H), 1.01-0.83 (m, 1H), 0.66-0.51 (m, 2H), 0.46 (dq, J=9.3, 4.8 Hz, 1H), 0.29 (dq, J=9.6, 4.8 Hz, 1H).

### Example 74—Synthesis of Compound 74

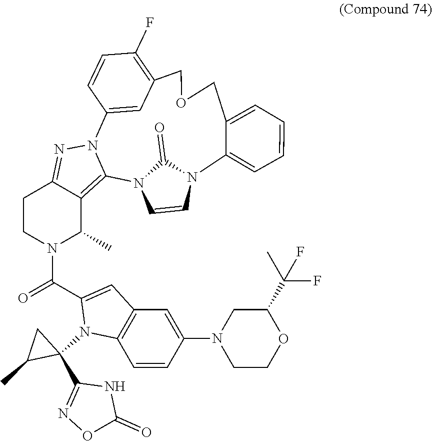

### (8S,10M)-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20), 16,18,24(28),25-nonaen-29-one

Compound 74 was synthesized following the procedure of Example 73, with(S)-2-cyclopropylmorpholine being replaced with (R)-2-(1,1-difluoroethyl)morpholine. m/z (ESI-pos) [M+1]=862.6; ¹H NMR (400 MHz, chloroform-D) δ 11.29 (d, J=20.5 Hz, 1H), 7.65-7.41 (m, 5H), 7.30 (dd, J=6.0, 2.8 Hz, 1H), 7.22-7.09 (m, 3H), 6.69 (d, J=33.3 Hz, 1H), 6.43 (dd, J=3.1, 1.1 Hz, 1H), 6.31-6.23 (m, 1H), 5.80 (q, J=6.5 Hz, 1H), 4.82-4.63 (m, 2H), 4.47 (dd, J=13.8, 5.0 Hz, 1H), 4.35-4.22 (m, 1H), 4.19-4.06 (m, 3H), 3.87 (dd, J=12.1, 9.2 Hz, 2H), 3.61 (dd, J=29.1, 12.0 Hz, 2H), 3.37 (d, J=12.0 Hz, 1H), 3.19-3.07 (m, 1H), 3.07-2.98 (m, 1H), 2.92-2.83 (m, 1H), 2.77 (t, J=11.0 Hz, 1H), 2.03 (d, J=1.1 Hz, 2H), 1.94-1.47 (m, 10H), 1.25 (td, J=7.1, 1.1 Hz, 3H), 1.22-1.17 (m, 2H).

### Example 75—Synthesis of Compound 75

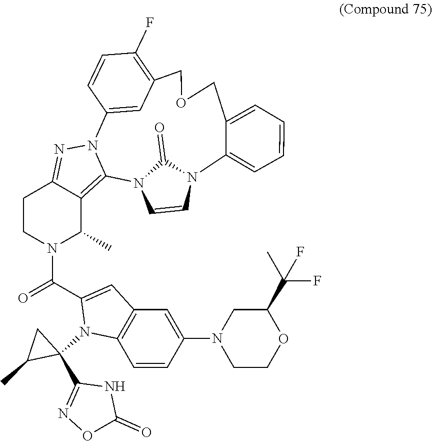

### (8S,10M)-7-({5-[(S)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20), 16,18,24(28),25-nonaen-29-one

Compound 75 was synthesized following the procedure of Example 73, with(S)-2-cyclopropylmorpholine being replaced by(S)-2-(1,1-difluoroethyl)morpholine. m/z (ESI-pos) [M+1]=862.6; ¹H NMR (400 MHz, chloroform-D) δ 7.65-7.42 (m, 5H), 7.30 (dd, J=6.1, 2.8 Hz, 1H), 7.22-7.10 (m, 3H), 6.70 (d, J=33.6 Hz, 1H), 6.43 (d, J=3.0 Hz, 1H), 6.27 (dd, J=11.5, 3.0 Hz, 1H), 5.91-5.75 (m, 1H), 4.82-4.63 (m, 2H), 4.47 (dd, J=13.8, 5.2 Hz, 1H), 4.35-4.22 (m, 1H), 4.19-4.06 (m, 2H), 3.87 (dd, J=11.9, 9.2 Hz, 2H), 3.69-3.54 (m, 2H), 3.36 (d, J=11.9 Hz, 1H), 3.19-3.08 (m, 1H), 3.03 (dd, J=15.9, 3.9 Hz, 1H), 2.88 (td, J=11.8, 3.3 Hz, 1H), 2.77 (t, J=11.1 Hz, 1H), 2.05-1.61 (m, 6H), 1.60-1.47 (m, 1H), 1.22-1.11 (m, 3H).

### Example 76—Synthesis of Compound 76

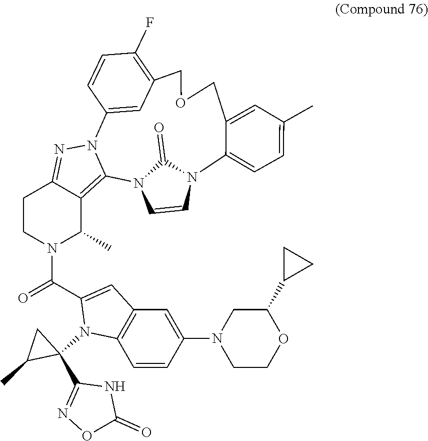

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20), 16,18,24(28),25-nonaen-29-one

Compound 76 was synthesized following the procedure of Examples 69 and 73, with (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one being replaced by (24S,31R,33R)-14-fluoro-24,44-dimethyl-24,25,26,27,32,33-hexahydro-22H,31H-6-oxa-2(2,3)-pyrazolo[4,3-c]pyridina-3(1,3)-imidazola-1(1,3),4(1,2)-dibenzenacycloheptaphan-32-one. m/z (ESI-pos) [M+1]=852.7; ¹H NMR (400 MHz, chloroform-D) δ 11.33 (s, 1H), 7.64-7.50 (m, 2H), 7.37-7.28 (m, 4H), 7.19-7.11 (m, 2H), 6.75-6.63 (m, 1H), 6.39 (d, J=3.0 Hz, 1H), 6.23 (dd, J=13.1, 3.1 Hz, 1H), 5.87-5.74 (m, 1H), 4.81-4.67 (m, 3H), 4.47 (dd, J=13.8, 5.0 Hz, 1H), 4.28-4.15 (m, 1H), 4.11 (d, J=9.2 Hz, 1H), 4.04 (d, J=11.3 Hz, 1H), 3.87-3.73 (m, 1H), 3.69-3.57 (m, 1H), 3.56-3.48 (m, 1H), 3.34 (d, J=11.8 Hz, 1H), 3.21-3.07 (m, 1H), 3.07-2.98 (m, 1H), 2.97-2.89 (m, 1H), 2.88-2.80 (m, 1H), 2.76-2.66 (m, 1H), 2.40 (d, J=8.3 Hz, 4H), 1.88 (t, J=6.0 Hz, 1H), 1.70-1.58 (m, 3H), 1.57-1.48 (m, 1H), 1.19 (d, J=5.9 Hz, 3H), 1.01-0.84 (m, 1H), 0.58 (dtd, J=22.4, 8.8, 4.2 Hz, 2H), 0.45 (dq, J=9.2, 4.8 Hz, 1H), 0.29 (dq, J=9.6, 4.8 Hz, 1H).

### Example 77—Synthesis of Compound 77

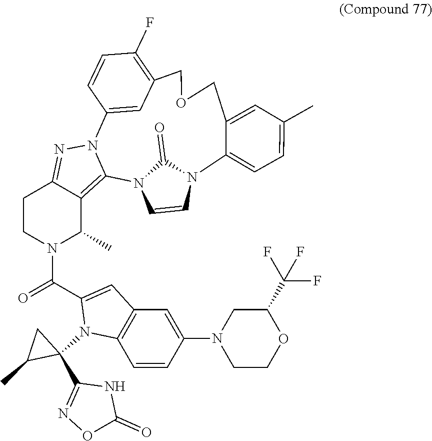

### (8S,10M)-25-fluoro-8,18-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20), 16,18,24(28),25-nonaen-29-one

Compound 77 was synthesized following the procedure of Examples 69 and 76, with(S)-2-cyclopropylmorpholine being replaced by (R)-2-(trifluoromethyl)morpholine. m/z (ESI-pos) [M+1]=880.6; ¹H NMR (400 MHz, chloroform-D) δ 11.30 (s, 1H), 7.64-7.52 (m, 2H), 7.39-7.26 (m, 3H), 7.23-7.08 (m, 3H), 6.77-6.64 (m, 1H), 6.39 (d, J=3.0 Hz, 1H), 6.27-6.18 (m, 1H), 5.87-5.74 (m, 1H), 4.81-4.63 (m, 2H), 4.46 (dd, J=13.7, 5.0 Hz, 1H), 4.25 (d, J=9.2 Hz, 1H), 4.19-4.09 (m, 3H), 3.90 (td, J=11.4, 2.6 Hz, 1H), 3.63 (td, J=12.9, 4.1 Hz, 1H), 3.54 (d, J=11.5 Hz, 1H), 3.36 (d, J=12.1 Hz, 1H), 3.19-3.06 (m, 1H), 3.06-2.80 (m, 3H), 2.40 (d, J=8.6 Hz, 3H), 1.90 (t, J=6.2 Hz, 1H), 1.66 (d, J=6.7 Hz, 3H), 1.63-1.47 (m, 1H), 1.21-1.12 (m, 3H)

### Example 78—Synthesis of Compound 78

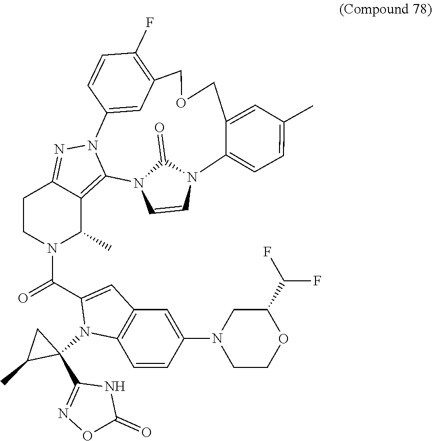

### (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20), 16,18,24(28),25-nonaen-29-one

Compound 78 was synthesized following the procedure of Compounds 69 and 76, with(S)-2-cyclopropylmorpholine being replaced by (R)-2-(difluoromethyl)morpholine. m/z (ESI-pos) [M+1]=862.6; ¹H NMR (400 MHz, chloroform-D) δ 11.31 (s, 1H), 7.94-7.51 (m, 2H), 7.39-7.28 (m, 3H), 7.21-7.08 (m, 2H), 6.79-6.62 (m, 1H), 6.39 (dd, J=3.0, 0.5 Hz, 1H), 6.27-6.19 (m, 1H), 5.98-5.64 (m, 2H), 4.81-4.63 (m, 2H), 4.52-4.34 (m, 1H), 4.29-4.16 (m, 1H), 4.15-4.08 (m, 2H), 4.01-3.83 (m, 2H), 3.62 (td, J=13.1, 4.0 Hz, 1H), 3.49 (d, J=11.5 Hz, 1H), 3.36 (d, J=11.8 Hz, 1H), 3.20-3.07 (m, 1H), 3.02 (dd, J=15.6, 3.8 Hz, 1H), 2.91 (td, J=11.6, 3.3 Hz, 1H), 2.80 (t, J=11.0 Hz, 1H), 2.40 (d, J=8.7 Hz, 3H), 1.94-1.81 (m, 1H), 1.73 (dd, J=51.8, 6.7 Hz, 3H), 1.62-1.47 (m, 1H), 1.23-1.11 (m, 3H).

### Example 79—Synthesis of Compound 79

### Synthetic Scheme

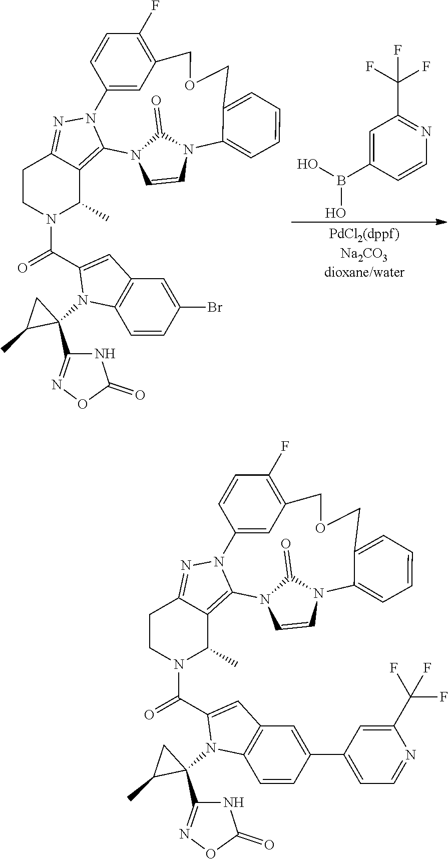

### Procedure for Synthesis of Compound 79

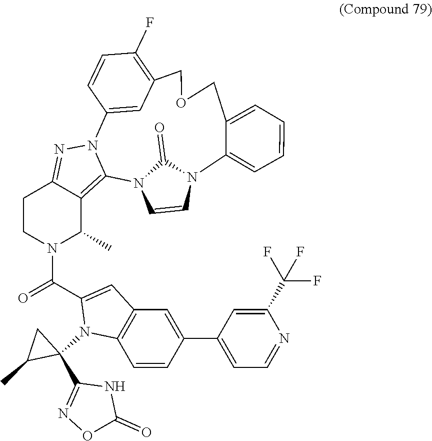

### (8S,10M)-25-fluoro-8-methyl-7-({1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: A solution of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (12 mg, 15.2 μmol), PdCl2(dppf) (1.24 mg, 1.52 μmol), [2-(trifluoromethyl)-4-pyridyl]boranediol (5.79 mg, 30.3 μmol) and 2M Na₂CO₃ (38 μL, 75 μmol) in dioxane (0.1 mL) was heated to 90° C. for 1 hour. The solution was then purified directly by reverse-phase chromatography (5-95% MeCN/water) and concentrated to give (8S,10M)-25-fluoro-8-methyl-7-({1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (11 mg, 12.8 μmol). m/z (ESI-pos) [M+1]=858.5; ¹H NMR (400 MHz, chloroform-D) δ 11.25 (s, 1H), 8.77 (d, J=5.1 Hz, 1H), 8.05-7.96 (m, 1H), 7.93 (d, J=1.8 Hz, 1H), 7.79 (d, J=8.6 Hz, 1H), 7.73 (dd, J=5.1, 1.8 Hz, 1H), 7.66 (dd, J=8.7, 1.8 Hz, 1H), 7.61 (dt, J=8.9, 3.6 Hz, 1H), 7.57-7.53 (m, 1H), 7.52-7.43 (m, 2H), 7.31 (dd, J=6.0, 2.8 Hz, 1H), 7.19 (t, J=9.0 Hz, 1H), 6.95-6.81 (m, 1H), 6.44 (d, J=3.1 Hz, 1H), 6.31-6.22 (m, 1H), 5.91-5.78 (m, 1H), 4.82-4.63 (m, 2H), 4.47 (dd, J=13.8, 5.0 Hz, 1H), 4.35-4.21 (m, 1H), 4.20-4.12 (m, 1H), 3.74-3.62 (m, 1H), 3.23-2.98 (m, 2H), 2.01-1.93 (m, 1H), 1.69 (d, J=6.7 Hz, 3H), 1.65-1.54 (m, 1H), 1.27-1.14 (m, 3H).

### Example 80—Synthesis of Compound 80

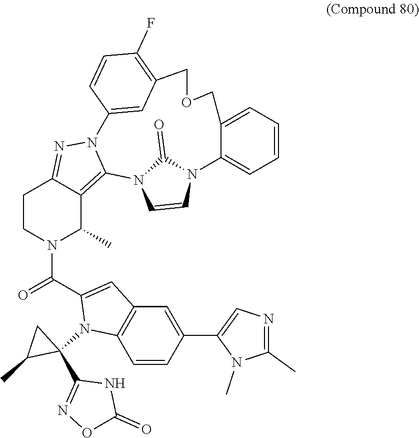

### (8S,10M)-25-fluoro-8-methyl-7-{[5-(1-methyl-2-methyl-5-imidazolyl)-1-[(1S,2S)-2-methyl-1-(5-oxo-4,5-dihydro-1,2,4-oxadiazol-3-yl)cyclopropyl]-2-indolyl]carbonyl}-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 80 was synthesized following the procedure of Example 79, with [2-(trifluoromethyl)-4-pyridyl]boranediol being replaced by 4,4,5,5-tetramethyl-2-(1-methyl-2-methyl-5-imidazolyl)-1,3,2-dioxaborolane. m/z (ESI-pos) [M+1]=807.6; ¹H NMR (400 MHz, chloroform-D) δ 7.76 (d, J=8.6 Hz, 1H), 7.67 (s, 1H), 7.60 (dd, J=8.0, 3.8 Hz, 1H), 7.56 (dd, J=6.9, 2.2 Hz, 1H), 7.52-7.42 (m, 3H), 7.34-7.27 (m, 1H), 7.23-7.11 (m, 2H), 6.84 (d, J=34.5 Hz, 1H), 6.44 (d, J=3.0 Hz, 1H), 6.28 (d, J=3.1 Hz, 1H), 5.93-5.78 (m, 1H), 4.82-4.68 (m, 3H), 4.48-4.39 (m, 1H), 4.34-4.21 (m, 1H), 4.16 (d, J=9.2 Hz, 1H), 3.73-3.61 (m, 1H), 3.59 (s, 3H), 3.13 (ddd, J=28.3, 20.3, 15.7 Hz, 2H), 2.69 (s, 4H), 1.69 (d, J=6.7 Hz, 3H), 1.57 (dd, J=9.5, 5.6 Hz, 1H), 1.26-1.20 (m, 6H)

### Example 81—Synthesis of Compound 81

### Synthetic Scheme

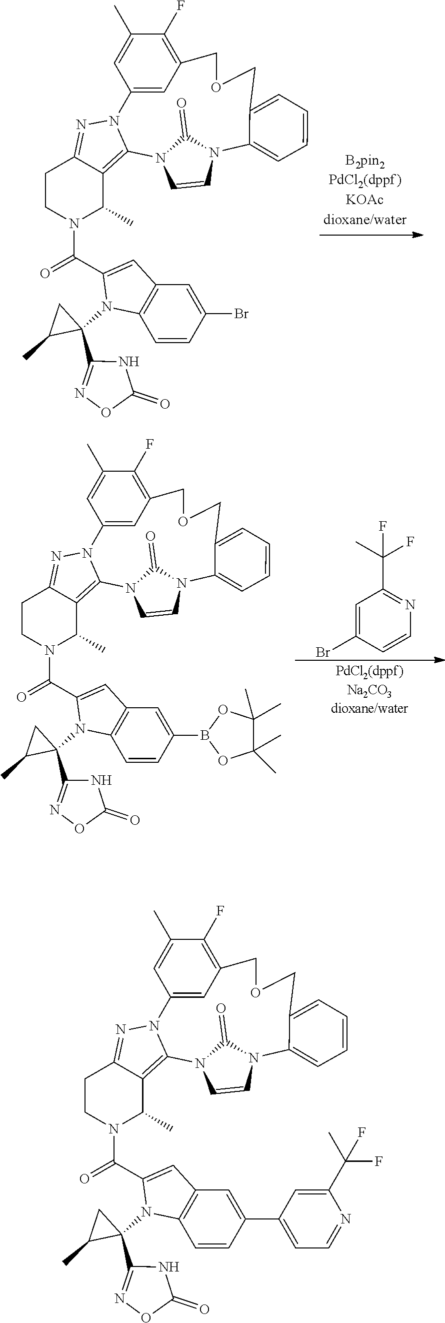

### Procedure for Synthesis of Compound 81

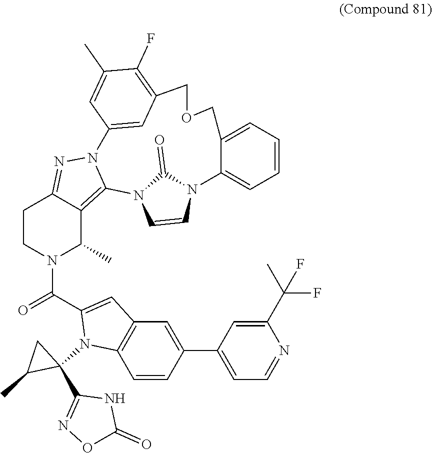

### (8S,10M)-7-({5-[2-(1,1-difluoroethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: A solution of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (104 mg, 129 μmol), PdCl₂ (dppf) (10.5 mg, 12.9 μmol), B₂pin₂ (65.4 mg, 258 μmol), and potassium acetate (63.2 mg, 644 μmol) in of dioxane (1 mL) was sparged with Argon and heated to 85° C. for 1 hour. The solution was then filtered through celite, concentrated, and purified by silica gel chromatography (1-10% MeOH/DCM) to give (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-(4,4,5,5-tetramethyl-1,3,2-dioxaborolan-2-yl)-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (94 mg, 110 μmol). m/z (ESI-pos) [M+1]=853.5

Step B: A solution of (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-(4,4,5,5-tetramethyl-1,3,2-dioxaborolan-2-yl)-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (25 mg, 29.3 μmol), PdCl2(dppf) (2.39 mg, 2.93 μmol), 4-bromo-2-(1,1-difluoroethyl)pyridine (13 mg, 58.6 μmol), and 2M disodium carbonate (147 μL, 293 μmol) in dioxane (0.2 mL) was sparged with Argon and heated to 90° C. for 1 hour. The solution was then filtered and purified by reverse-phase chromatography (5-95% MeCN/water, 0.1% FA). The product was concentrated to give (8S,10M)-7-({5-[2-(1,1-difluoroethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (17 mg, 19.6 μmol). m/z (ESI-pos) [M+1]=868.6; ¹H NMR (400 MHz, chloroform-D) δ 11.27 (s, 1H), 8.69 (dd, J=5.2, 0.8 Hz, 1H), 7.99 (dd, J=1.8, 0.7 Hz, 1H), 7.91 (dd, J=1.8, 0.8 Hz, 1H), 7.80-7.72 (m, 1H), 7.70-7.64 (m, 1H), 7.61 (dd, J=5.2, 1.9 Hz, 1H), 7.58-7.41 (m, 4H), 7.11 (dd, J=5.6, 2.8 Hz, 1H), 6.93-6.80 (m, 1H), 6.44 (d, J=3.0 Hz, 1H), 6.26 (dd, J=16.8, 3.0 Hz, 1H), 5.91-5.78 (m, 1H), 4.81-4.62 (m, 2H), 4.47 (dd, J=13.8, 4.9 Hz, 1H), 4.30 (d, J=9.3 Hz, 1H), 4.16 (d, J=9.3 Hz, 1H), 3.67 (td, J=13.1, 4.1 Hz, 1H), 3.22-3.10 (m, 1H), 3.07 (dd, J=16.1, 4.1 Hz, 1H), 2.36-2.29 (m, 3H), 2.07 (t, J=18.7 Hz, 3H), 1.95 (dt, J=22.7, 6.3 Hz, 1H), 1.75 (dd, J=56.0, 6.7 Hz, 3H), 1.66-1.55 (m, 1H), 1.26-1.13 (m, 3H).

### Example 82—Synthesis of Compound 82

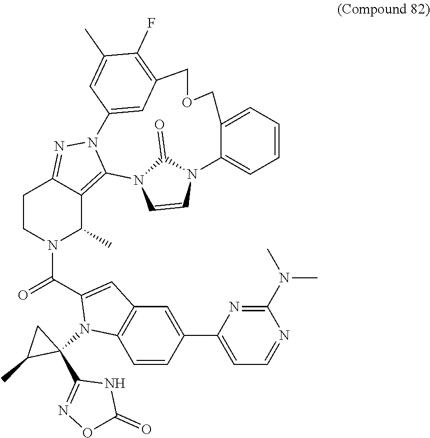

### (8S,10M)-7-({5-[2-(dimethylamino)-4-pyrimidinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 82 was synthesized following the procedure of Example 81, with 4-bromo-2-(1,1-difluoroethyl)pyridine replaced by N,N-dimethyl (4-bromo-2-pyrimidinyl)amine. m/z (ESI-pos) [M+1]=848.6; ¹H NMR (400 MHz, chloroform-D) δ 11.29 (s, 1H), 8.47-8.40 (m, 1H), 8.37 (d, J=5.2 Hz, 1H), 8.15-8.03 (m, 1H), 7.70 (dd, J=12.3, 8.7 Hz, 1H), 7.58-7.41 (m, 4H), 7.11 (dd, J=5.6, 2.8 Hz, 1H), 6.98-6.88 (m, 1H), 6.83 (s, 1H), 6.44 (d, J=3.0 Hz, 1H), 6.26 (dd, J=18.9, 3.1 Hz, 1H), 5.88-5.77 (m, 1H), 4.81-4.61 (m, 2H), 4.49 (dd, J=13.9, 5.0 Hz, 1H), 4.31 (d, J=9.3 Hz, 1H), 4.15 (dd, J=9.1, 5.2 Hz, 1H), 3.72-3.60 (m, 1H), 3.29 (d, J=1.9 Hz, 6H), 3.22-3.11 (m, 1H), 3.10-2.98 (m, 1H), 2.36-2.31 (m, 3H), 1.96 (t, J=6.2 Hz, 1H), 1.71-1.60 (m, 3H), 1.60-1.51 (m, 1H), 1.18 (dd, J=30.4, 6.1 Hz, 3H)

### Example 83—Synthesis of Compound 83

### Synthetic Scheme

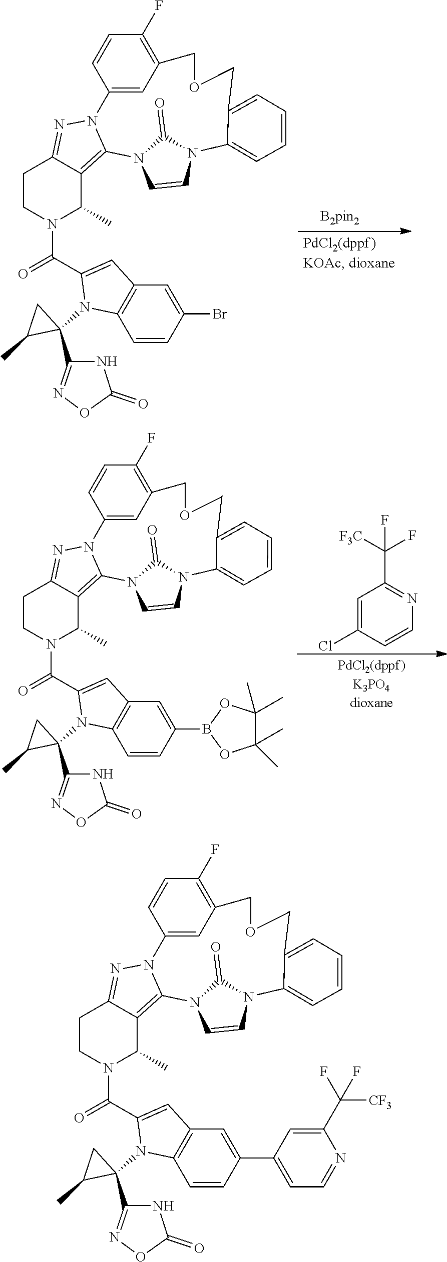

### Procedure for Synthesis of Compound 83

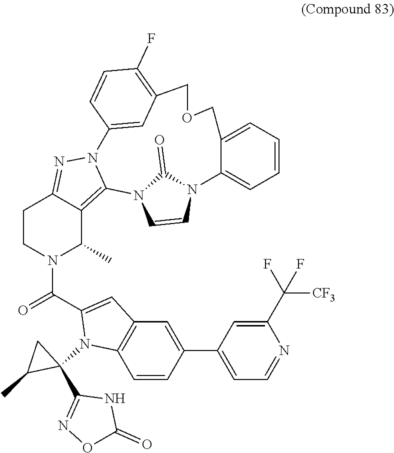

### (8S,10M)-25-fluoro-8-methyl-7-({1-[(1S,2S)-2-methyl-1-(5-oxo-4,5-dihydro-1,2,4-oxadiazol-3-yl)cyclopropyl]-5-[2-(1,1,2,2,2-pentafluoroethyl)-4-pyridyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: A solution of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (125 mg, 158 μmol)\, PdCl2(dppf) (12.9 mg, 15.8 μmol), B₂pin₂ (80.2 mg, 316 μmol), and potassium acetate (77.5 mg, 790 μmol) in 1 mL of dioxane was sparged with Argon and heated to 85° C. for 1 hour. The solution was cooled to ambient temperature and then filtered through celite, concentrated, and purified by silica gel chromatography (1-10% MeOH/DCM) to give (8S,10M)-25-fluoro-8-methyl-7-({1-[(1S,2S)-2-methyl-1-(5-oxo-4,5-dihydro-1,2,4-oxadiazol-3-yl)cyclopropyl]-5-(4,4,5,5-tetramethyl-1,3,2-dioxaborolan-2-yl)-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (120 mg, 129 μmol). m/z (ESI-pos) [M+1]=839.5

Step B: A solution of (8S,10M)-25-fluoro-8-methyl-7-({1-[(1S,2S)-2-methyl-1-(5-oxo-4,5-dihydro-1,2,4-oxadiazol-3-yl)cyclopropyl]-5-(4,4,5,5-tetramethyl-1,3,2-dioxaborolan-2-yl)-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (17.4 mg, 20.7 μmol), PdCl2(dppf) (1.69 mg, 2.07 μmol), 4-chloro-2-(1,1,2,2,2-pentafluoroethyl)pyridine (9.59 mg, 41.4 μmol) and K₃PO₄ (22 mg, 104 μmol) in 1:1 dioxane/water (0.2 mL) was sparged with Argon and heated to 95° C. for 1 hour. The solution was then diluted with MeOH, filtered, and purified directly by reverse-phase chromatography (5-95% MeCN/water, 0.1% formic acid), and concentrated to give (8S,10M)-25-fluoro-8-methyl-7-({1-[(1S,2S)-2-methyl-1-(5-oxo-4,5-dihydro-1,2,4-oxadiazol-3-yl)cyclopropyl]-5-[2-(1,1,2,2,2-pentafluoroethyl)-4-pyridyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (4 mg, 4.41 μmol). m/z (ESI-pos) [M+1]=908.5; ¹H NMR (400 MHz, chloroform-D) δ 11.24 (s, 1H), 8.80 (d, J=5.1 Hz, 1H), 8.04-7.96 (m, 1H), 7.94 (s, 1H), 7.80 (d, J=8.6 Hz, 1H), 7.74 (s, OH), 7.66 (dd, J=8.7, 1.8 Hz, 1H), 7.64-7.59 (m, 0.5H), 7.56 (dd, J=6.8, 2.2 Hz, 0.5H), 7.53-7.43 (m, 3H), 7.31 (dd, J=6.1, 2.7 Hz, 1H), 7.18 (t, J=9.0 Hz, 1H), 6.88 (d, J=35.2 Hz, 1H), 6.44 (d, J=3.0 Hz, 1H), 6.30-6.22 (m, 1H), 5.91-5.79 (m, 1H), 4.82-4.67 (m, 2H), 4.46 (dd, J=13.8, 5.0 Hz, 1H), 4.35-4.22 (m, 1H), 4.17 (d, J=9.2 Hz, 1H), 3.73-3.62 (m, 1H), 3.22-3.11 (m, 1H), 3.11-3.01 (m, 1H), 1.99 (d, J=4.8 Hz, 1H), 1.72-1.62 (m, 3H), 1.57 (dd, J=9.6, 5.6 Hz, 1H), 1.24 (d, J=6.0 Hz, 3H)

### Example 84—Synthesis of Compound 84

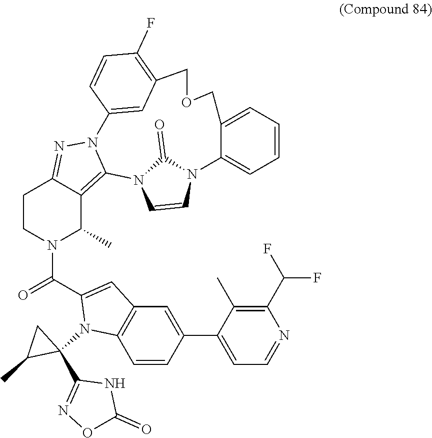

### (8S,10M)-7-({5-[2-(difluoromethyl)-3-methyl-4-pyridyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28), 3,9,12,15,17,19,24,26-nonaen-29-one

Compound 84 was synthesized following the procedure of Example 83, with 4-chloro-2-(1,1,2,2,2-pentafluoroethyl)pyridine being replaced by 4-bromo-2-(difluoromethyl)-3-methylpyridine. m/z (ESI-pos) [M+1]=854.6; ¹H NMR (400 MHz, chloroform-D) δ 11.29 (s, 1H), 8.48 (d, J=4.9 Hz, 1H), 7.73 (d, J=8.5 Hz, 1H), 7.63-7.42 (m, 5H), 7.33-7.27 (m, 2H), 7.18 (t, J=8.9 Hz, 1H), 6.97-6.64 (m, 2H), 6.44 (d, J=3.0 Hz, 1H), 6.28 (d, J=3.1 Hz, 1H), 5.83 (q, J=6.7 Hz, 1H), 4.82-4.64 (m, 2H), 4.48 (dd, J=13.7, 5.1 Hz, 1H), 4.35-4.22 (m, 1H), 4.16 (d, J=9.2 Hz, 1H), 3.67 (td, J=13.0, 4.1 Hz, 1H), 3.23-3.10 (m, 1H), 3.06 (dd, J=16.3, 3.9 Hz, 1H), 2.42 (d, J=1.9 Hz, 3H), 2.00-1.91 (m, 1H), 1.69 (d, J=6.7 Hz, 3H), 1.64-1.56 (m, 1H), 1.24 (d, J=5.7 Hz, 3H)

### Example 85—Synthesis of Compound 85

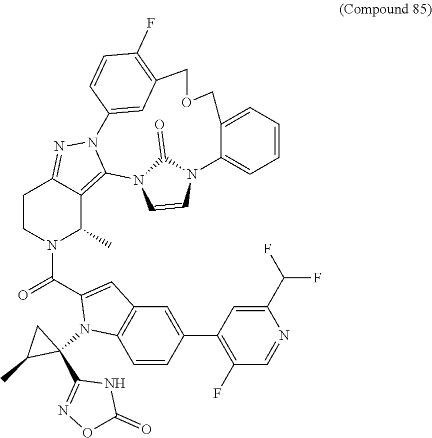

### (8S,10M)-7-({5-[2-(difluoromethyl)-5-fluoro-4-pyridyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 85 was synthesized following the procedure of Example 83, with 4-chloro-2-(1,1,2,2,2-pentafluoroethyl)pyridine being replaced by 4-bromo-2-(difluoromethyl)-5-fluoropyridine. m/z (ESI-pos) [M+1]=858.5; ¹H NMR (400 MHz, chloroform-D) δ 11.25 (s, 1H), 8.56 (s, 1H), 7.98 (d, J=12.2 Hz, 1H), 7.83-7.73 (m, 3H), 7.62 (dd, J=8.7, 6.2 Hz, 2H), 7.57-7.52 (m, 1H), 7.52-7.42 (m, 2H), 7.34 (dd, J=19.1, 4.6 Hz, 1H), 7.18 (t, J=8.9 Hz, 1H), 6.83 (s, 1H), 6.73-6.40 (m, 2H), 6.27 (dd, J=9.9, 3.1 Hz, 1H), 5.92-5.78 (m, 1H), 4.82-4.63 (m, 2H), 4.47 (dd, J=13.8, 5.0 Hz, 1H), 4.28 (dd, J=25.8, 9.1 Hz, 1H), 4.16 (d, J=9.1 Hz, 1H), 3.73-3.62 (m, 1H), 3.23-3.02 (m, 2H), 1.95 (dt, J=23.3, 6.2 Hz, 1H), 1.73-1.60 (m, 3H), 1.60-1.39 (m, 1H), 1.20 (dd, J=28.9, 6.1 Hz, 3H)

### Example 86—Synthesis of Compound 86

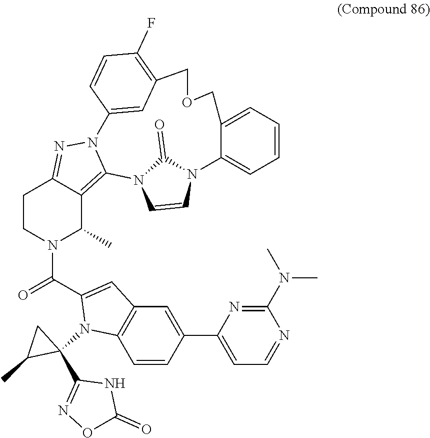

### (8S,10M)-7-({5-[2-(dimethylamino)-4-pyrimidinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 86 was synthesized following the procedure of Example 83, with 4-chloro-2-(1,1,2,2,2-pentafluoroethyl)pyridine replaced by N,N-dimethyl (4-bromo-2-pyrimidinyl)amine. m/z (ESI-pos) [M+1]=834.6; ¹H NMR (400 MHz, chloroform-D) δ 8.48-8.39 (m, 1H), 8.37 (d, J=5.2 Hz, 1H), 8.15-8.03 (m, 1H), 7.71 (d, J=8.8 Hz, 1H), 7.62 (dt, J=8.3, 3.7 Hz, 1H), 7.58-7.44 (m, 3H), 7.39-7.27 (m, 1H), 7.18 (t, J=8.9 Hz, 1H), 6.95 (d, J=5.2 Hz, 1H), 6.83 (s, 1H), 6.44 (d, J=3.0 Hz, 1H), 6.26 (dd, J=15.6, 3.1 Hz, 1H), 5.88-5.78 (m, 1H), 4.82-4.63 (m, 3H), 4.54-4.46 (m, 1H), 4.28 (dd, J=26.3, 9.0 Hz, 1H), 4.16 (q, J=7.8 Hz, 1H), 3.66 (t, J=11.4 Hz, 1H), 3.29 (s, 7H), 3.21-2.99 (m, 2H), 1.98-1.79 (m, 2H), 1.68 (d, J=6.8 Hz, 3H), 1.61-1.51 (m, 1H), 1.22 (d, J=6.0 Hz, 3H)

### Example 87—Synthesis of Compound 87

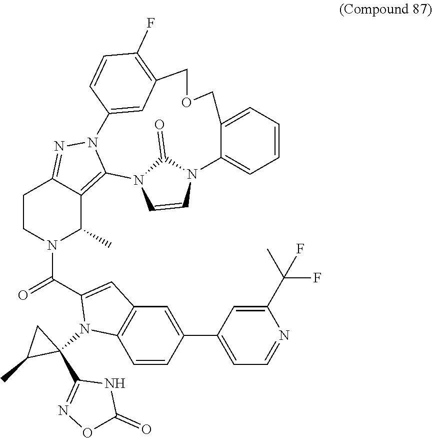

### (8S,10M)-7-({5-[2-(1,1-difluoroethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 87 was synthesized following the procedure of Example 83, 4-chloro-2-(1,1,2,2,2-pentafluoroethyl)pyridine replaced by 4-bromo-2-(1,1-difluoroethyl)pyridine. m/z (ESI-pos) [M+1]=854.5; ¹H NMR (400 MHz, chloroform-D) δ 11.26 (s, 1H), 8.69 (d, J=5.1 Hz, 1H), 8.04-7.96 (m, 1H), 7.91 (dd, J=1.8, 0.8 Hz, 1H), 7.77 (d, J=8.6 Hz, 1H), 7.67 (dd, J=8.7, 1.8 Hz, 1H), 7.65-7.59 (m, 1H), 7.55 (dd, J=6.9, 2.2 Hz, 1H), 7.52-7.41 (m, 3H), 7.31 (dd, J=6.0, 2.8 Hz, 1H), 7.18 (t, J=8.9 Hz, 1H), 6.93-6.80 (m, 1H), 6.44 (d, J=3.0 Hz, 1H), 6.31-6.22 (m, 1H), 5.83 (q, J=6.7 Hz, 1H), 4.82-4.63 (m, 3H), 4.48 (dd, J=13.7, 5.0 Hz, 1H), 4.29 (dd, J=26.4, 9.2 Hz, 1H), 4.16 (d, J=9.3 Hz, 1H), 3.74-3.62 (m, 1H), 3.23-3.13 (m, 1H), 3.07 (dd, J=15.9, 3.9 Hz, 1H), 2.07 (t, J=18.7 Hz, 3H), 1.98 (t, J=6.1 Hz, 1H), 1.68 (t, J=7.4 Hz, 3H), 1.62-1.53 (m, 1H), 1.23 (d, J=6.1 Hz, 3H)

### Example 88—Synthesis of Compound 88

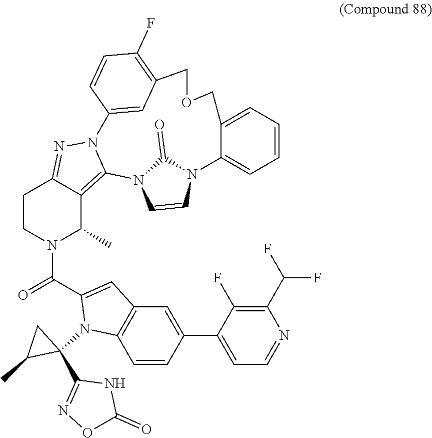

### (8S,10M)-7-({5-[2-(difluoromethyl)-3-fluoro-4-pyridyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 88 was synthesized following the procedure of Example 83, with 4-chloro-2-(1,1,2,2,2-pentafluoroethyl)pyridine replaced by 4-bromo-2-(difluoromethyl)-3-fluoropyridine m/z (ESI-pos) [M+1]=858.5; ¹H NMR (400 MHz, chloroform-D) δ 11.25 (s, 1H), 8.52 (d, J=4.9 Hz, 1H), 7.97 (d, J=11.9 Hz, 1H), 7.78 (d, J=8.6 Hz, 1H), 7.66-7.41 (m, 6H), 7.31 (dd, J=6.1, 2.8 Hz, 1H), 7.18 (t, J=9.0 Hz, 1H), 7.04-6.72 (m, 2H), 6.44 (d, J=3.0 Hz, 1H), 6.27 (dd, J=8.4, 3.1 Hz, 1H), 5.93-5.77 (m, 1H), 4.82-4.68 (m, 2H), 4.47 (dd, J=14.0, 5.1 Hz, 1H), 4.29 (dd, J=25.9, 9.2 Hz, 1H), 4.16 (d, J=9.2 Hz, 1H), 3.74-3.62 (m, 1H), 3.13 (dtd, J=31.7, 16.2, 4.7 Hz, 2H), 1.95 (dt, J=24.3, 6.4 Hz, 1H), 1.69 (d, J=6.8 Hz, 3H), 1.60 (qd, J=9.5, 4.3 Hz, 1H), 1.23 (d, J=5.9 Hz, 3H)

### Example 89—Synthesis of Compound 89

### Synthetic Scheme

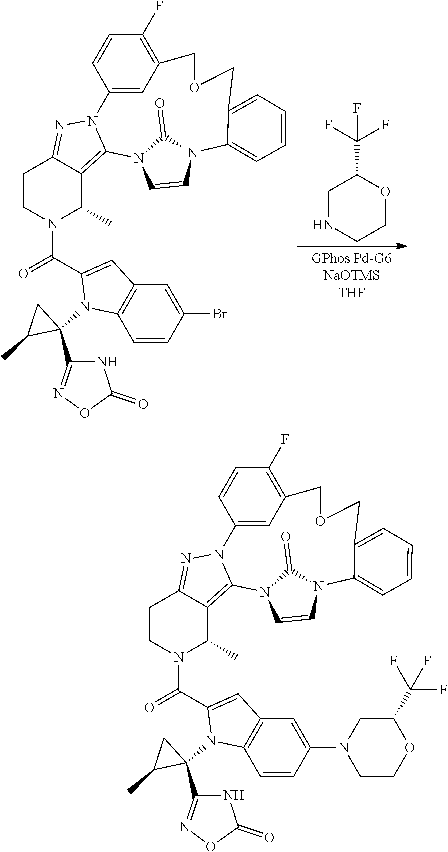

### Procedure for the Synthesis of Compound 89

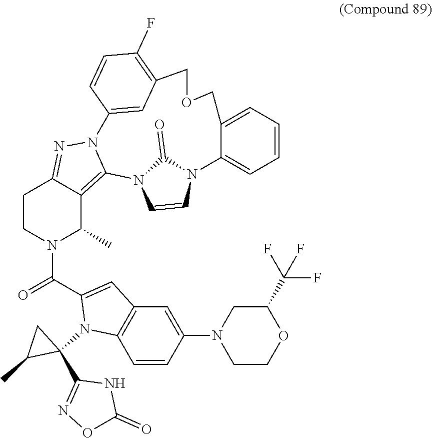

### (8S,10M)-25-fluoro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20), 16,18,24(28),25-nonaen-29-one

Step A: A 4 mL vial was charged with (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (20 mg, 25.9 μmol), [(R)-2-(trifluoromethyl)-4λ⁴-4-morpholinium-4-yl]-22-chloranide (18.8 mg, 98.5 μmol), GPhos Pd-G6 (2.44 mg, 2.59 μmol), sodium trimethylsilanolate (11.6 mg, 103 μmol), followed by addition of tetrahydrofuran (646 μL). The mixture was sparged for 5 minutes with Argon and then heated to 70° C. overnight. The next morning the solution was filtered and purified by reverse-phase chromatography (5-95% MeCN/water, 0.1% FA). The product was concentrated to give (8S,10M)-25-fluoro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20), 16,18,24(28),25-nonaen-29-one (24 mg, 27.7 μmol). m/z (ESI-pos) [M+1]=866.6; ¹H NMR (400 MHz, chloroform-D) δ 11.30 (s, 1H), 7.65-7.41 (m, 5H), 7.38-7.27 (m, 1H), 7.22-7.08 (m, 3H), 6.66 (s, 1H), 6.44 (d, J=3.0 Hz, 1H), 6.26 (dd, J=10.1, 3.0 Hz, 1H), 5.80 (q, J=6.6 Hz, 1H), 4.82-4.67 (m, 2H), 4.46 (dd, J=13.8, 5.0 Hz, 1H), 4.40-4.22 (m, 1H), 4.15 (d, J=9.6 Hz, 4H), 3.90 (td, J=11.4, 2.6 Hz, 1H), 3.63 (td, J=13.1, 4.1 Hz, 1H), 3.54 (d, J=11.3 Hz, 1H), 3.36 (d, J=12.0 Hz, 1H), 3.20-2.82 (m, 4H), 1.93-1.76 (m, 2H), 1.66 (d, J=6.7 Hz, 3H), 1.63-1.47 (m, 2H), 1.20 (d, J=6.0 Hz, 3H).

### Example 90—Synthesis of Compound 90

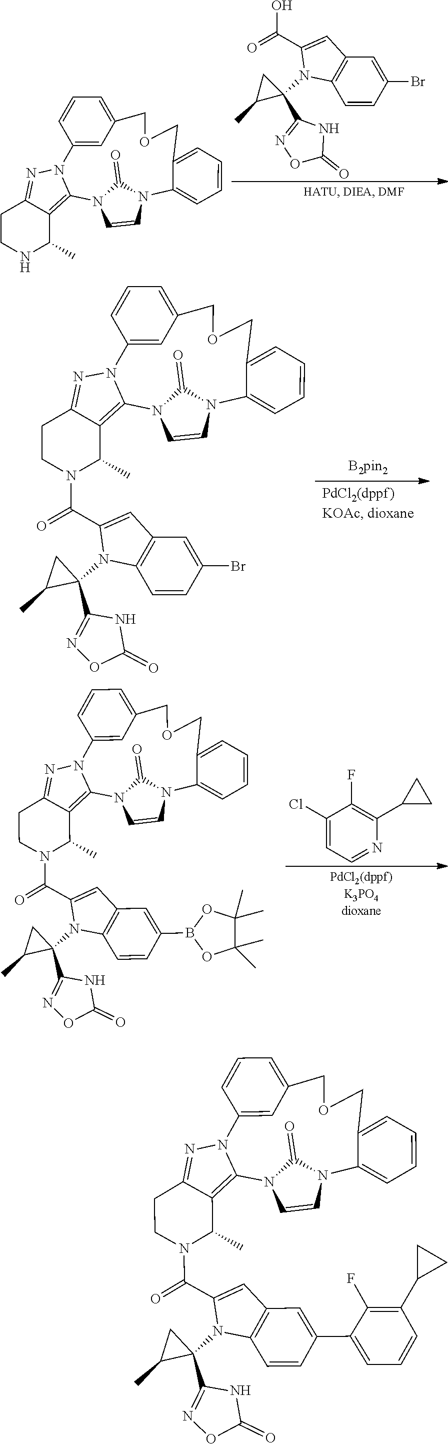

### Procedure for the Synthesis of Compound 90

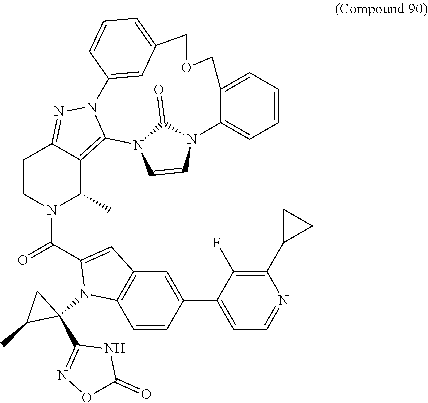

### (8S,10M)-7-{[5-(2-cyclopropyl-3-fluoro-4-pyridyl)-1-[(1S,2S)-2-methyl-1-(5-oxo-4,5-dihydro-1,2,4-oxadiazol-3-yl)cyclopropyl]-2-indolyl]carbonyl}-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (8S,10M)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (215 mg, 478 μmol) and 5-bromo-1-[(1S,2S)-2-methyl-1-(5-oxo-4,5-dihydro-1,2,4-oxadiazol-3-yl)cyclopropyl]-2-indolecarboxylic acid (199 mg, 526 μmol) in dimethylformamide (2.39 mL) was added N-ethyldiisopropylamine (416 μL, 2.39 mmol) and HATU (363 mg, 956 μmol). The mixture stirred at ambient temperature for 72 hours. The product was directly purified by Prep HPLC (Isco ACCQ Prep, Gemini column NX—C18, 250×50 mm, 20 ml/min, 25 minute separation×2 injections, 40 to 80% H₂O/ACN with 0.1% Formic acid buffer), pure fractions were collected and concentrated to give (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (306 mg, 396 μmol). m/z (ESI-pos) [M+1]=773.4, 775.4

Step B: A solution of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (180 mg, 233 μmol), PdCl₂(dppf) (19 mg, 23.3 μmol), B₂pin₂ (118 mg, 465 μmol), and potassium acetate (114 mg, 1.16 mmol) in 2.4 mL of dioxane was sparged with Argon and heated to 80° C. overnight. The solution was then cooled to ambient temperature and filtered through celite, concentrated, and purified by silica gel chromatography (1-10% MeOH/DCM) to give (8S,10M)-8-methyl-7-({1-[(1S,2S)-2-methyl-1-(5-oxo-4,5-dihydro-1,2,4-oxadiazol-3-yl)cyclopropyl]-5-(4,4,5,5-tetramethyl-1,3,2-dioxaborolan-2-yl)-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (130 mg, 158 μmol). m/z (ESI-pos) [M+1]=821.6

Step C: A solution of (8S,10M)-8-methyl-7-({1-[(1S,2S)-2-methyl-1-(5-oxo-4,5-dihydro-1,2,4-oxadiazol-3-yl)cyclopropyl]-5-(4,4,5,5-tetramethyl-1,3,2-dioxaborolan-2-yl)-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (25 mg, 30.5 μmol), PdCl₂(dppf) (2.49 mg, 3.05 μmol), 4-chloro-2-cyclopropyl-3-fluoropyridine (10.5 mg, 60.9 μmol) and K₃PO₄ (32.3 mg, 152 μmol) in 0.2 mL of 1:1 dioxane/water was sparged with Argon and heated to 95° C. for 1 hour. The solution was then cooled to ambient temperature, diluted with MeOH, filtered, and purified directly by reverse-phase chromatography (5-95% MeCN/water) and concentrated to give product which was further purified by silica gel chromatography (50-100% EA/heptanes) to give (8S,10M)-7-{[5-(2-cyclopropyl-3-fluoro-4-pyridyl)-1-[(1S,2S)-2-methyl-1-(5-oxo-4,5-dihydro-1,2,4-oxadiazol-3-yl)cyclopropyl]-2-indolyl]carbonyl}-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (7 mg, 8.01 μmol). m/z (ESI-pos) [M+1]=830.5; ¹H NMR (400 MHz, chloroform-D) δ 11.28 (d, J=19.6 Hz, 1H), 8.27 (d, J=4.9 Hz, 1H), 7.93 (d, J=11.8 Hz, 1H), 7.74 (t, J=8.8 Hz, 1H), 7.68-7.57 (m, 2H), 7.54-7.41 (m, 5H), 7.21 (d, J=7.6 Hz, 1H), 7.13 (dd, J=5.8, 4.9 Hz, 1H), 6.92-6.77 (m, 1H), 6.45 (d, J=3.0 Hz, 1H), 6.30 (d, J=3.1 Hz, 1H), 5.89-5.80 (m, 1H), 4.67 (s, 2H), 4.49 (dd, J=13.7, 5.2 Hz, 1H), 4.27 (dd, J=27.3, 9.3 Hz, 1H), 4.16-4.06 (m, 2H), 3.67 (td, J=13.1, 4.0 Hz, 1H), 3.23-3.03 (m, 2H), 2.45-2.39 (m, OH), 1.97 (t, J=5.8 Hz, 1H), 1.68 (d, J=6.7 Hz, 3H), 1.23 (dd, J=6.5, 3.4 Hz, 3H), 1.20-1.13 (m, 3H), 1.06 (dt, J=8.3, 3.1 Hz, 2H)

### Example 91—Synthesis of Compound 91

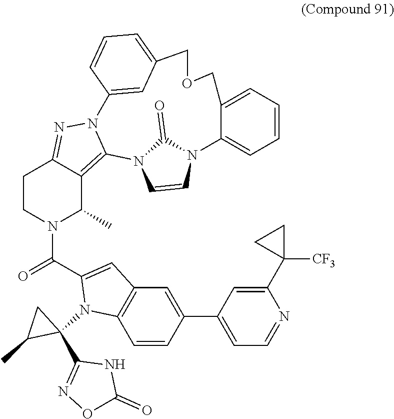

Compound 91 was synthesized following the procedure of Example 90, with 4-chloro-2-cyclopropyl-3-fluoropyridine replaced with 4-bromo-2-[1-(trifluoromethyl)cyclopropyl]pyridine. m/z (ESI-pos) [M+1]=880.6; ¹H NMR (400 MHz, chloroform-D) δ 11.28 (s, 1H), 8.59 (d, J=5.2 Hz, 1H), 8.00-7.92 (m, 1H), 7.81-7.71 (m, 2H), 7.68-7.61 (m, 2H), 7.56-7.40 (m, 6H), 7.23-7.14 (m, 1H), 6.83 (s, 1H), 6.45 (dd, J=3.1, 0.6 Hz, 1H), 6.35-6.20 (m, 1H), 5.90-5.79 (m, 1H), 4.67 (s, 2H), 4.48 (dd, J=13.9, 5.0 Hz, 1H), 4.27 (dd, J=26.6, 9.3 Hz, 1H), 4.10 (d, J=9.4 Hz, 1H), 3.68 (td, J=13.2, 4.2 Hz, 1H), 3.25-3.04 (m, 2H), 1.99-1.80 (m, 1H), 1.69 (d, J=6.7 Hz, 3H), 1.64-1.53 (m, 1H), 1.19 (dd, J=29.6, 6.1 Hz, 3H)

### Example 92—Synthesis of Compound 92

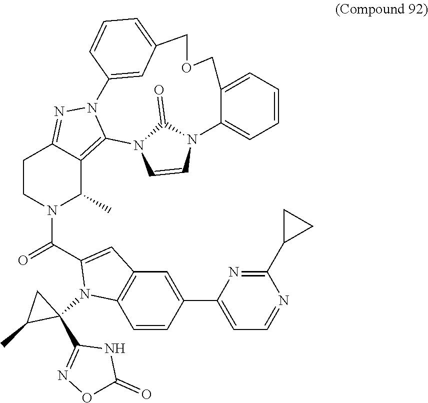

### (8S,10M)-7-({[5-(2-(cyclopropyl)-4-pyrimidinyl]-1-[(1S,2S)-2-methyl-1-(5-oxo-4,5-dihydro-1,2,4-oxadiazol-3-yl)cyclopropyl]-2-indolyl]carbonyl}-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 92 was synthesized following the procedure of Example 90, with 4-chloro-2-cyclopropyl-3-fluoropyridine replaced by 4-chloro-2-cyclopropylpyrimidine. m/z (ESI-pos) [M+1]=813.6; ¹H NMR (400 MHz, chloroform-D) δ 11.29 (s, 1H), 8.58 (d, J=5.4 Hz, 1H), 8.48 (d, J=12.9 Hz, 1H), 8.12-7.99 (m, 1H), 7.73 (d, J=8.8 Hz, 1H), 7.65 (d, J=8.1 Hz, 1H), 7.55-7.40 (m, 6H), 7.23-7.14 (m, 1H), 7.00-6.83 (m, 1H), 6.51-6.42 (m, 1H), 6.27 (dd, J=24.1, 3.1 Hz, 1H), 5.87-5.79 (m, 1H), 4.67 (s, 2H), 4.52-4.44 (m, 1H), 4.34-4.21 (m, 1H), 4.10 (d, J=9.3 Hz, 1H), 3.73-3.62 (m, 1H), 3.17 (dd, J=12.4, 5.1 Hz, 1H), 3.08 (d, J=17.7 Hz, 1H), 2.32 (dq, J=8.2, 4.2 Hz, 1H), 2.01-1.89 (m, 1H), 1.82 (d, J=6.8 Hz, 1H), 1.68 (d, J=6.6 Hz, 3H), 1.23 (d, J=6.1 Hz, 3H), 1.18-1.07 (m, 2H).

### Example 93—Synthesis of Compound 93

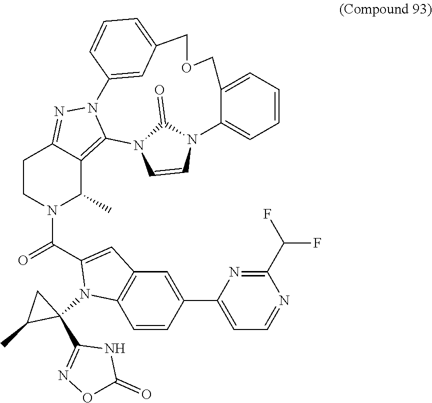

### (8S,10M)-7-({5-[2-(difluoromethyl)-4-pyrimidinyl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 93 was synthesized following the procedure of Example 90, with 4-chloro-2-cyclopropyl-3-fluoropyridine replaced by 4-bromo-2-(difluoromethyl)pyrimidine m/z (ESI-pos) [M+1]=823.5; ¹H NMR (400 MHz, chloroform-D) δ 11.28 (s, 1H), 8.86 (d, J=5.4 Hz, 1H), 8.66-8.59 (m, 1H), 8.15-8.02 (m, 1H), 7.84 (d, J=5.4 Hz, 1H), 7.77 (d, J=8.8 Hz, 1H), 7.70-7.58 (m, 1H), 7.54-7.41 (m, 5H), 7.21 (d, J=7.6 Hz, 1H), 6.89-6.83 (m, 1H), 6.65 (d, J=54.7 Hz, 1H), 6.45 (d, J=3.0 Hz, 1H), 6.30 (d, J=3.0 Hz, 1H), 5.88-5.79 (m, 1H), 4.67 (s, 2H), 4.47 (dd, J=13.9, 5.3 Hz, 1H), 4.27 (dd, J=26.1, 9.3 Hz, 1H), 4.10 (d, J=9.2 Hz, 1H), 3.75-3.58 (m, 2H), 3.24-3.00 (m, 2H), 2.00-1.80 (m, 1H), 1.69 (d, J=6.8 Hz, 3H), 1.23 (d, J=6.0 Hz, 3H)

### Example 94—Synthesis of Compound 94

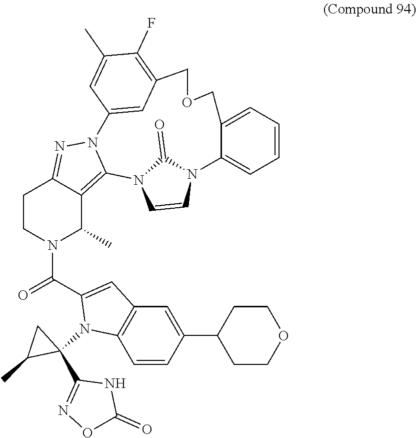

### (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-(tetrahydro-4H-pyran-4-yl)-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 94 was synthesized following the procedure of Example 66, with 5-bromo-1-((1S,2S)-2-methyl-1-(5-oxo-4,5-dihydro-1,2,4-oxadiazol-3-yl)cyclopropyl)-1H-indole-2-carboxylic acid being replaced by 1-[(1S,2S)-2-methyl-1-(5-oxo-4,5-dihydro-1,2,4-oxadiazol-3-yl)cyclopropyl]-5-(tetrahydro-4H-pyran-4-yl)-2-indolecarboxylic acid. m/z (ESI-pos) [M+1]=811.5; ¹H NMR (400 MHz, chloroform-D) δ 11.29 (d, J=18.7 Hz, 1H), 7.60 (d, J=8.5 Hz, 1H), 7.57-7.52 (m, 1H), 7.52-7.42 (m, 4H), 7.28 (d, J=1.7 Hz, 1H), 7.10 (dd, J=5.7, 2.8 Hz, 1H), 6.70 (d, J=0.8 Hz, 1H), 6.44 (d, J=3.0 Hz, 1H), 6.28 (d, J=3.0 Hz, 1H), 5.80 (q, J=6.6 Hz, 1H), 4.81-4.62 (m, 3H), 4.46 (dd, J=13.8, 5.1 Hz, 1H), 4.30 (d, J=9.3 Hz, 1H), 4.26-4.06 (m, 4H), 3.67-3.44 (m, 4H), 3.21-3.08 (m, 1H), 3.03 (dd, J=15.6, 3.8 Hz, 1H), 2.91-2.81 (m, 1H), 2.34-2.29 (m, 3H), 1.94-1.77 (m, 6H), 1.66 (d, J=6.7 Hz, 3H), 1.20 (d, J=5.8 Hz, 3H)

### Example 95—Synthesis of Compound 95

### Synthetic Scheme

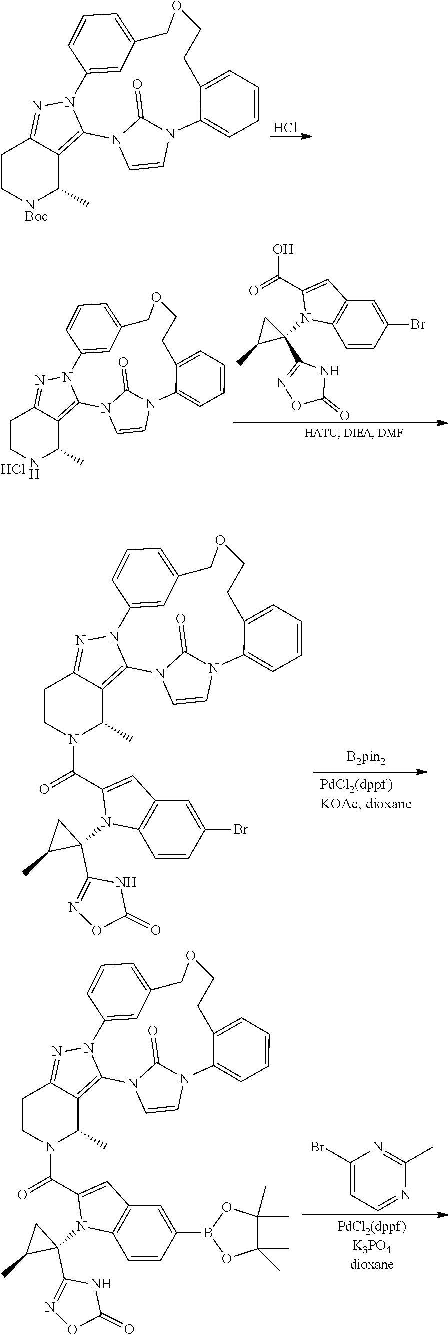

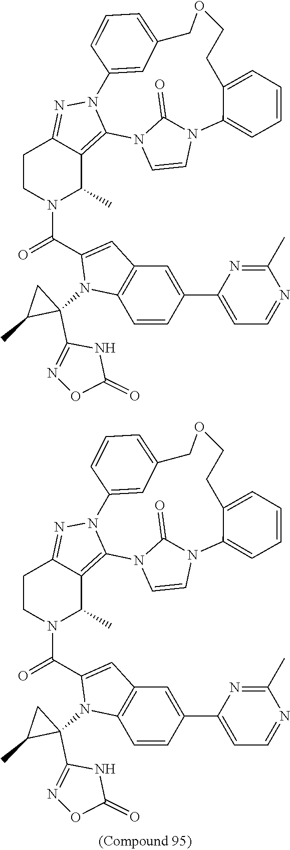

### (S)-8-methyl-7-({1-[(1S,2S)-2-methyl-1-(5-oxo-4,5-dihydro-1,2,4-oxadiazol-3-yl)cyclopropyl]-5-(2-methyl-4-pyrimidinyl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: A solution of tert-butyl (S)-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(28),3,9,12,15(20),16,18,25(29),26-nonaene-7-carboxylate (550 mg, 1.04 mmol) in 5 mL of 4M HCl in dioxane was stirred for 1 hour and concentrated to give (S)-8-methyl-23-oxa-2,3,7,11,14 pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (480 mg, 1.03 mmol) which was used directly in the subsequent step. m/z (ESI-pos) M+1=428.3

Step B: A solution of (S)-8-methyl-23-oxa-2,3,7,11,14 pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one hydrogen chloride (480 mg, 1.03 mmol), 5-bromo-1-[(1S,2S)-2-methyl-1-(5-oxo-4,5-dihydro-1,2,4-oxadiazol-3-yl)cyclopropyl]-2-indolecarboxylic acid (469 mg, 1.24 mmol), HATU (590 mg, 1.55 mmol), and DIEA (541 μL, 3.1 mmol) in 1 mL of DMF was stirred for 1 hour at 50° C. The solution was then taken up in EtOAc and washed with water (3×). The organics were concentrated and purified directly by silica gel chromatography (10-94% EtOAc/heptanes) to give (S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (550 mg, 663 μmol). m/z (ESI-pos) M+1=787.4, 789.3

Step C: A solution of (S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (20 mg, 25.4 μmol), PdCl₂(dppf) (2.07 mg, 2.54 μmol), B₂pin₂ (12.9 mg, 50.8 μmol), and potassium acetate (12.5 mg, 127 μmol) in 0.4 mL of dioxane was sparged with Argon and heated to 80° C. overnight. The solution was then filtered through celite, concentrated, and purified by silica gel chromatography (1-10% MeOH/DCM) to give (S)-8-methyl-7-({1-[(1S,2S)-2-methyl-1-(5-oxo-4,5-dihydro-1,2,4-oxadiazol-3-yl)cyclopropyl]-5-(4,4,5,5-tetramethyl-1,3,2-dioxaborolan-2-yl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (13 mg, 15.6 μmol).

Step D: A solution of (S)-8-methyl-7-({1-[(1S,2S)-2-methyl-1-(5-oxo-4,5-dihydro-1,2,4-oxadiazol-3-yl)cyclopropyl]-5-(4,4,5,5-tetramethyl-1,3,2-dioxaborolan-2-yl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (10.6 mg, 12.7 μmol), PdCl₂(dppf) (1.04 mg, 1.27 μmol), 4-bromo-2-methylpyrimidine (4.39 mg, 25.4 μmol) and disodium carbonate (6.73 mg, 63.5 μmol) in 0.1 mL of dioxane was heated to 90° C. for 1 hour. The solution was cooled to ambient temperature and then purified directly by reverse-phase chromatography (5-95% MeCN/water) and to give (S)-8-methyl-7-({1-[(1S,2S)-2-methyl-1-(5-oxo-4,5-dihydro-1,2,4-oxadiazol-3-yl)cyclopropyl]-5-(2-methyl-4-pyrimidinyl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (3 mg, 3.75 μmol). m/z (ESI-pos) [M+1]=801.5; ¹H NMR (400 MHz, chloroform-D) δ 11.25 (d, J=17.4 Hz, 1H), 8.68-8.59 (m, 1H), 8.51-8.42 (m, 1H), 8.10-8.03 (m, 1H), 7.77-7.69 (m, 1H), 7.58 (d, J=8.0 Hz, 1H), 7.54 (d, J=5.3 Hz, 1H), 7.48 (td, J=7.7, 2.6 Hz, 1H), 7.34 (d, J=2.7 Hz, 3H), 7.19 (dd, J=14.2, 9.4 Hz, 2H), 6.91-6.79 (m, 1H), 6.50-6.21 (m, 2H), 6.10-5.77 (m, 1H), 5.29 (d, J=0.9 Hz, 1H), 4.80-4.39 (m, 4H), 4.11 (qd, J=7.2, 0.9 Hz, 1H), 3.73-3.58 (m, 1H), 3.48 (d, J=3.8 Hz, 1H), 3.44-3.29 (m, 3H), 3.29-3.12 (m, 1H), 3.07 (dd, J=16.2, 3.7 Hz, 1H), 2.97-2.86 (m, 1H), 2.82-2.77 (m, 3H), 2.03 (d, J=0.9 Hz, 2H), 1.97 (t, J=6.1 Hz, 1H), 1.66 (d, J=6.7 Hz, 3H), 1.58 (m, 4H), 1.21 (d, J=6.1 Hz, 3H).

### Example 96—Synthesis of Compound 96

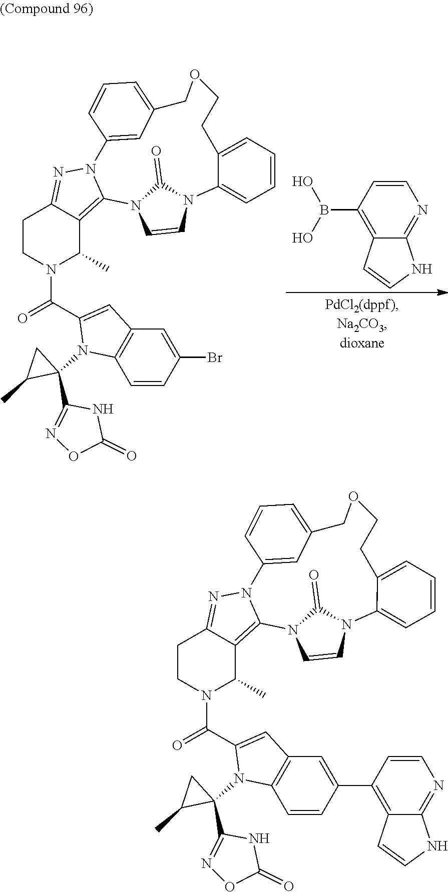

### (S)-7-{[5-(1,7-diaza-1H-inden-4-yl)-1-[(1S,2S)-2-methyl-1-(5-oxo-4,5-dihydro-1,2,4-oxadiazol-3-yl)cyclopropyl]-2-indolyl]carbonyl}-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: A solution of (S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (10 mg, 12.7 μmol), PdCl₂(dppf) (1.04 mg, 1.27 μmol), (1,7-diaza-1H-inden-4-yl)boranediol (4.11 mg, 25.4 μmol) and disodium carbonate (6.73 mg, 63.5 μmol) in 0.1 mL of dioxane was heated to 90° C. for 1 hour. The solution was then cooled to ambient temperature and purified directly by reverse-phase chromatography (5-95% MeCN/water) and concentrated to give (S)-7-{[5-(1,7-diaza-1H-inden-4-yl)-1-[(1S,2S)-2-methyl-1-(5-oxo-4,5-dihydro-1,2,4-oxadiazol-3-yl)cyclopropyl]-2-indolyl]carbonyl}-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (4 mg, 4.85 μmol). m/z (ESI-pos) [M+1]=825.6; ¹H NMR (400 MHz, chloroform-D) δ 9.10 (s, 1H), 8.36 (s, 1H), 8.04 (s, 1H), 7.77 (d, J=8.9 Hz, 2H), 7.58 (d, J=8.1 Hz, 1H), 7.48 (t, J=7.8 Hz, 1H), 7.39-7.31 (m, 4H), 7.23-7.13 (m, 2H), 6.92-6.81 (m, 1H), 6.75 (s, 1H), 6.50-6.24 (m, 2H), 5.83 (d, J=6.7 Hz, 1H), 4.76-4.44 (m, 2H), 3.76-3.60 (m, 1H), 3.46-3.15 (m, 2H), 3.08 (d, J=16.5 Hz, 1H), 2.97-2.75 (m, 1H), 2.05-1.95 (m, 1H), 1.64 (dd, J=18.5, 7.1 Hz, 4H), 1.23 (d, J=5.7 Hz, 3H).

### Example 97—Synthesis of Compound 97

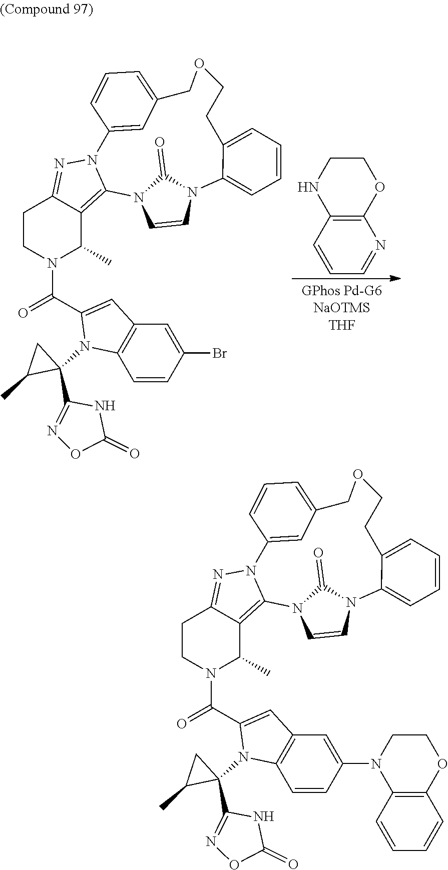

### (S)-8-methyl-7-({1-[(1S,2S)-2-methyl-1-(5-oxo-4,5-dihydro-1,2,4-oxadiazol-3-yl)cyclopropyl]-5-(4-oxa-1,5-diaza-2,3-dihydro-1H-naphth-1-yl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: A solution of (S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (17 mg, 21.6 μmol), GPhos Pd G6 (2.04 mg, 2.16 μmol), NaOTMS (9.68 mg, 86.3 μmol), and 4-oxa-1,5-diaza-2,3-dihydro-1H-naphthalene (5.9 mg, 43.2 μmol) in 0.3 mL of THF was sparged with Argon and then heated to 75° C. overnight and then cooled to ambient temperature. The reaction was then purified by silica gel chromatography (1-10% MeOH/dcm). The product was further purified by reverse-phase chromatography (5-95% MeCN/water, 0.1% FA) to give (S)-8-methyl-7-({1-[(1S,2S)-2-methyl-1-(5-oxo-4,5-dihydro-1,2,4-oxadiazol-3-yl)cyclopropyl]-5-(4-oxa-1,5-diaza-2,3-dihydro-1H-naphth-1-yl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (3 mg, 3.56 μmol). m/z (ESI-pos) [M+1]=843.5; ¹H NMR (400 MHz, chloroform-D) δ 11.26 (d, J=14.8 Hz, 1H), 7.70-7.61 (m, 2H), 7.57 (d, J=8.1 Hz, 1H), 7.54-7.40 (m, 2H), 7.37-7.32 (m, 2H), 7.30-7.26 (m, 1H), 7.19 (dd, J=12.7, 7.7 Hz, 2H), 6.96-6.87 (m, 1H), 6.73-6.59 (m, 2H), 6.50-6.27 (m, 2H), 6.26-5.74 (m, 1H), 4.82-4.57 (m, 2H), 4.55-4.40 (m, 4H), 3.76-3.58 (m, 3H), 3.36 (tt, J=11.5, 5.6 Hz, 3H), 3.21-2.86 (m, 4H), 1.94 (t, J=5.7 Hz, 1H), 1.66 (t, J=7.5 Hz, 3H), 1.21 (d, J=5.8 Hz, 3H)

### Example 98—Synthesis of Compound 98

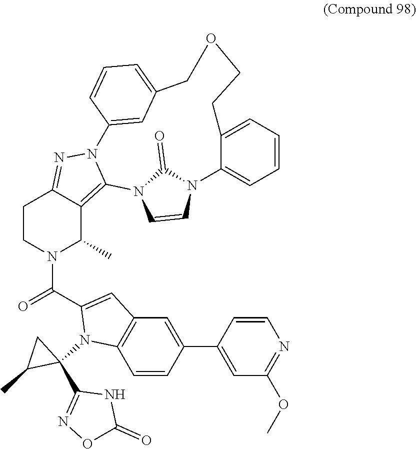

### (S)-7-{[5-(2-methoxy-4-pyridyl)-1-[(1S,2S)-2-methyl-1-(5-oxo-4,5-dihydro-1,2,4-oxadiazol-3-yl)cyclopropyl]-2-indolyl]carbonyl}-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 98 was synthesized following the procedure of Example 96, with (1,7-diaza-1H-inden-4-yl)boranediol replaced by (2-methoxy-4-pyridyl)boranediol. m/z (ESI-pos) [M+1]=816.6; ¹H NMR (400 MHz, chloroform-D) δ 11.24 (d, J=22.1 Hz, 1H), 8.23-8.19 (m, 1H), 7.92 (dd, J=1.8, 0.7 Hz, 1H), 7.76-7.68 (m, 1H), 7.62 (dd, J=8.7, 1.7 Hz, 1H), 7.61-7.57 (m, 1H), 7.48 (td, J=7.7, 3.8 Hz, 1H), 7.36-7.31 (m, 3H), 7.23-7.19 (m, 1H), 7.16-7.10 (m, 2H), 7.00-6.92 (m, 1H), 6.88-6.76 (m, 1H), 6.49-6.26 (m, 2H), 6.25-5.76 (m, 1H), 4.76-4.53 (m, 2H), 4.48 (dd, J=15.0, 5.2 Hz, 1H), 4.01-3.95 (m, 3H), 3.74-3.58 (m, 1H), 3.36 (qd, J=11.7, 7.0 Hz, 2H), 3.19 (ddd, J=21.7, 11.5, 5.2 Hz, 1H), 3.07 (dd, J=15.9, 3.7 Hz, 1H), 2.98-2.85 (m, 1H), 1.65 (d, J=6.7 Hz, 3H), 1.60 (dd, J=6.4, 4.0 Hz, 1H), 1.21 (d, J=6.1 Hz, 3H)

### Example 99—Synthesis of Compound 99

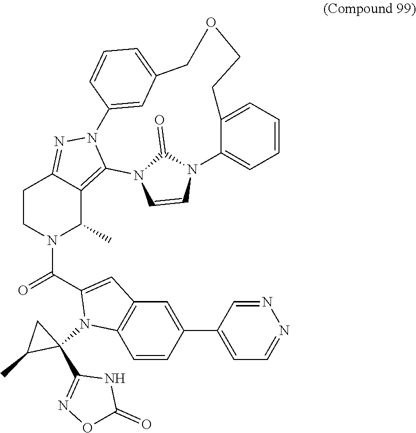

### (S)-8-methyl-7-({1-[(1S,2S)-2-methyl-1-(5-oxo-4,5-dihydro-1,2,4-oxadiazol-3-yl)cyclopropyl]-5-(4-pyridazinyl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 99 was synthesized following the procedure of Example 96, with (1,7-diaza-1H-inden-4-yl)boranediol replaced by 4,4,5,5-tetramethyl-2-(4-pyridazinyl)-1,3,2-dioxaborolane. m/z (ESI-pos) [M+1]=787.5; ¹H NMR (400 MHz, chloroform-D) δ 11.23 (s, 1H), 9.51 (s, 1H), 9.25-9.15 (m, 1H), 8.00 (d, J=1.9 Hz, 1H), 7.80 (t, J=8.8 Hz, 1H), 7.67 (d, J=1.7 Hz, 1H), 7.58 (ddd, J=8.1, 2.2, 1.2 Hz, 1H), 7.51-7.46 (m, 1H), 7.37-7.32 (m, 3H), 7.23-7.17 (m, 2H), 6.87-6.79 (m, 1H), 6.50-6.23 (m, 3H), 6.06-5.77 (m, 1H), 4.80-4.38 (m, 4H), 3.75-3.60 (m, 1H), 3.46-3.29 (m, 2H), 3.19 (ddd, J=17.5, 12.1, 5.3 Hz, 1H), 3.12-3.00 (m, 1H), 2.96-2.86 (m, 1H), 2.02-1.89 (m, 1H), 1.86-1.80 (m, OH), 1.68 (dd, J=14.8, 6.7 Hz, 3H), 1.61 (dd, J=6.5, 3.1 Hz, 1H), 1.22 (dd, J=6.1, 1.7 Hz, 3H).

### Example 100—Synthesis of Compound 100

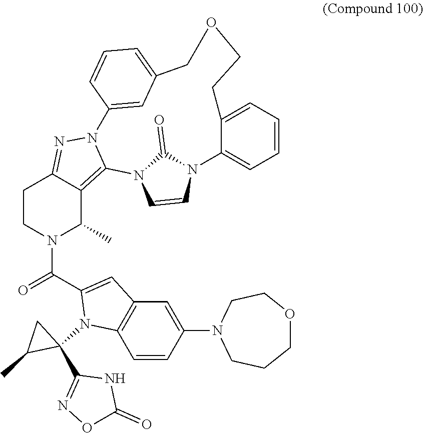

### (S)-8-methyl-7-({1-[(1S,2S)-2-methyl-1-(5-oxo-4,5-dihydro-1,2,4-oxadiazol-3-yl)cyclopropyl]-5-(1,4-oxazepan-4-yl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 100 was synthesized following the procedure of Example 97, with 4-oxa-1,5-diaza-2,3-dihydro-1H-naphthalene replaced by 1,4-oxazepane. m/z (ESI-pos) [M+1]=808.6; ¹H NMR (400 MHz, chloroform-D) δ 11.31 (d, J=21.0 Hz, 1H), 7.58 (d, J=8.0 Hz, 1H), 7.52-7.43 (m, 2H), 7.35-7.30 (m, 3H), 7.19 (t, J=10.2 Hz, 2H), 6.98-6.81 (m, 2H), 6.64-6.51 (m, 1H), 6.49-6.24 (m, 2H), 5.98-5.72 (m, 1H), 4.73-4.46 (m, 3H), 3.90-3.83 (m, 2H), 3.73-3.59 (m, 8H), 3.43-3.30 (m, 2H), 3.21-2.86 (m, 1H), 2.07 (p, J=5.9 Hz, 2H), 1.90-1.72 (m, 1H), 1.61 (dd, J=14.6, 6.7 Hz, 3H), 1.18 (d, J=5.7 Hz, 3H)

### Example 101—Synthesis of Compound 101

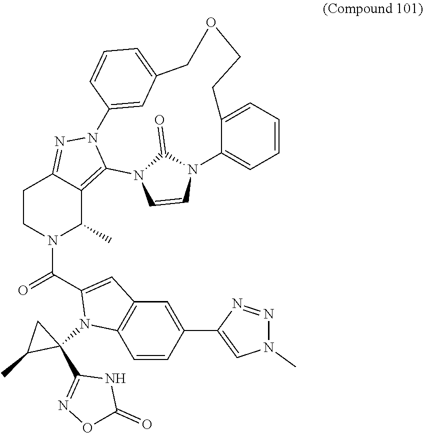

### (S)-8-methyl-7-({1-[(1S,2S)-2-methyl-1-(5-oxo-4,5-dihydro-1,2,4-oxadiazol-3-yl)cyclopropyl]-5-(1-methyl-1H-1,2,3-triazol-4-yl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 101 was synthesized following the procedure of Example 96, with (1,7-diaza-1H-inden-4-yl)boranediol replaced by (1-methyl-1H-1,2,3-triazol-4-yl)boranediol. m/z (ESI-pos) [M+1]=789.6; ¹H NMR (400 MHz, chloroform-D) δ 11.27 (d, J=21.1 Hz, 1H), 7.79-7.71 (m, 2H), 7.65-7.54 (m, 3H), 7.51-7.44 (m, 2H), 7.36-7.31 (m, 2H), 7.19 (dd, J=15.8, 7.7 Hz, 2H), 6.80-6.68 (m, 1H), 6.50-6.20 (m, 2H), 5.96-5.73 (m, 1H), 4.74-4.39 (m, 3H), 3.95 (d, J=1.6 Hz, 3H), 3.66 (td, J=13.4, 4.5 Hz, 1H), 3.45-3.32 (m, 3H), 3.20-2.84 (m, 2H), 1.96-1.74 (m, 2H), 1.65 (d, J=6.7 Hz, 3H), 1.20 (d, J=5.9 Hz, 3H)

### Example 102—Synthesis of Compound 102

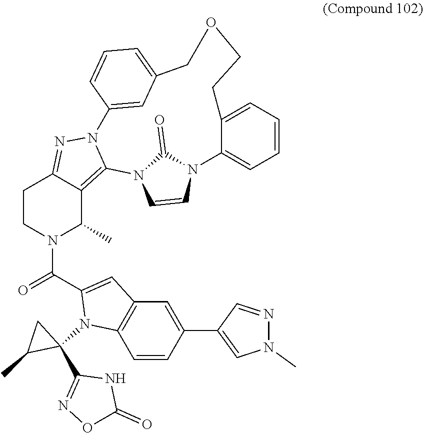

### (S)-8-methyl-7-({1-[(1S,2S)-2-methyl-1-(5-oxo-4,5-dihydro-1,2,4-oxadiazol-3-yl)cyclopropyl]-5-(1-methyl-4-pyrazolyl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 102 was synthesized following the procedure of Example 96, with (1,7-diaza-1H-inden-4-yl)boranediol replaced by (1-methyl-4-pyrazoyl)boranediol. m/z (ESI-pos) [M+1]=789.6; ¹H NMR (400 MHz, chloroform-D) δ 7.78-7.68 (m, 2H), 7.66-7.43 (m, 5H), 7.37-7.27 (m, 3H), 7.24-7.06 (m, 2H), 6.79-6.68 (m, 1H), 6.48-6.35 (m, 2H), 5.95-5.74 (m, 1H), 4.75-4.40 (m, 3H), 3.95 (d, J=1.7 Hz, 3H), 3.71-3.56 (m, 1H), 3.45-3.03 (m, 3H), 2.94 (s, 1H), 1.96-1.78 (m, 1H), 1.71-1.53 (m, 4H), 1.16 (dd, J=28.0, 6.1 Hz, 3H)

### Example 103—Synthesis of Compound 103

### Synthetic Scheme

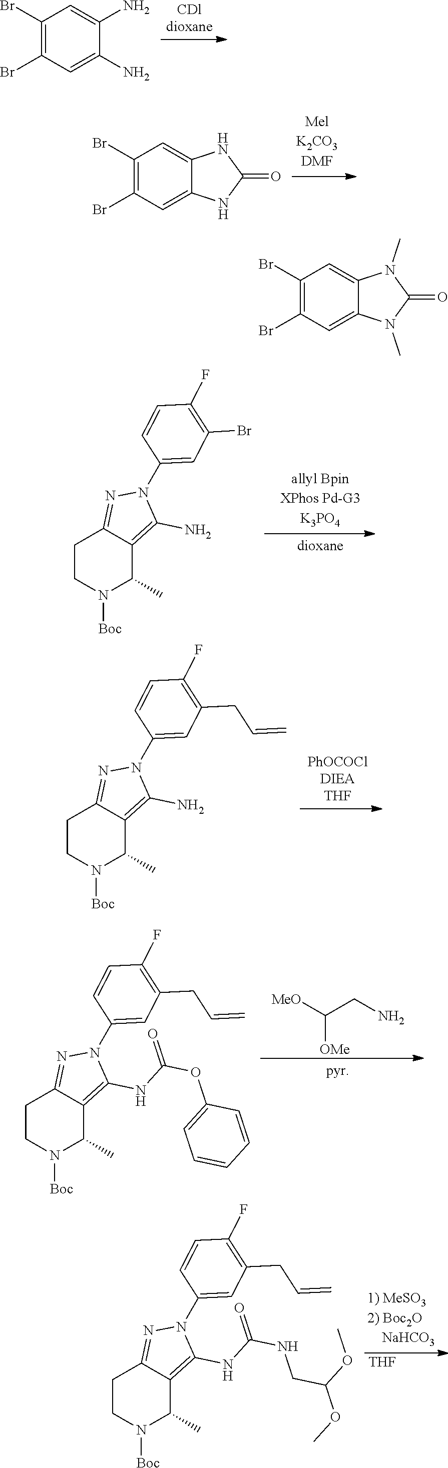

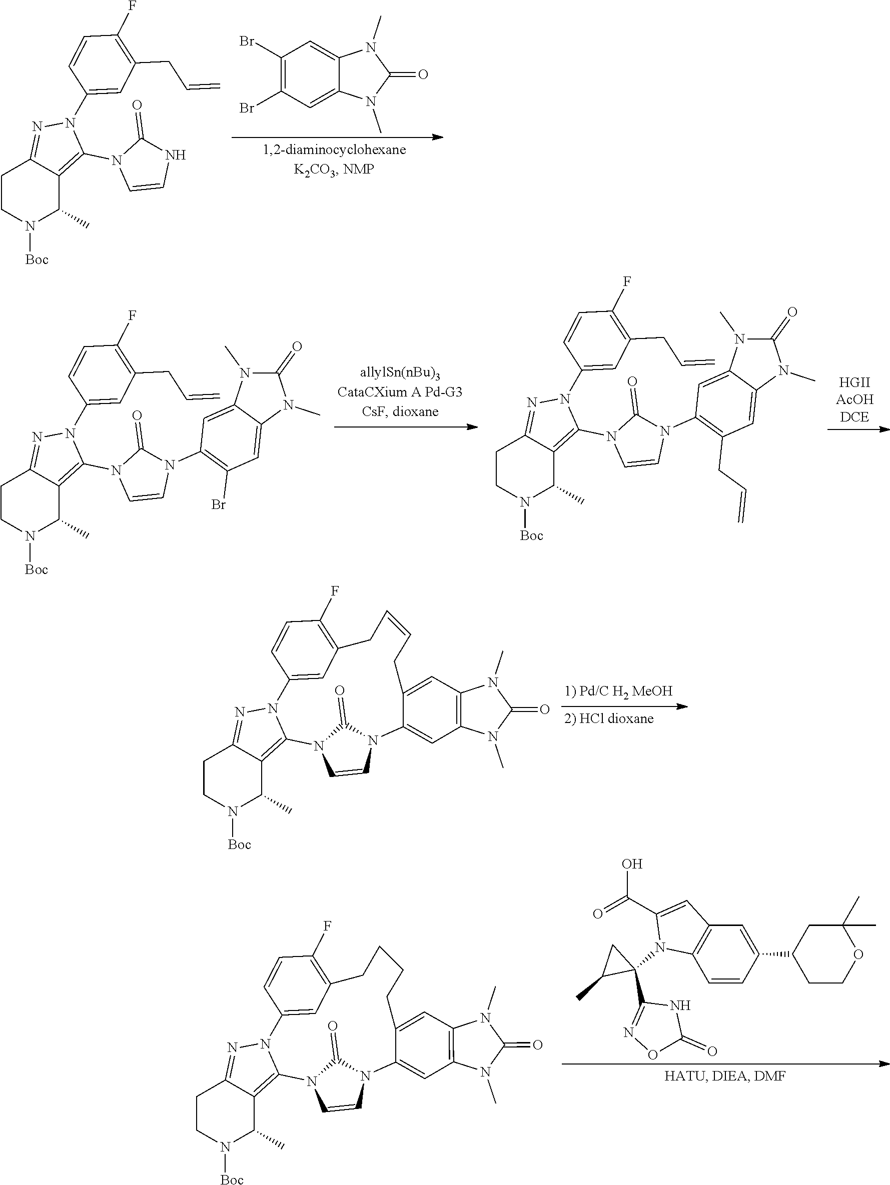

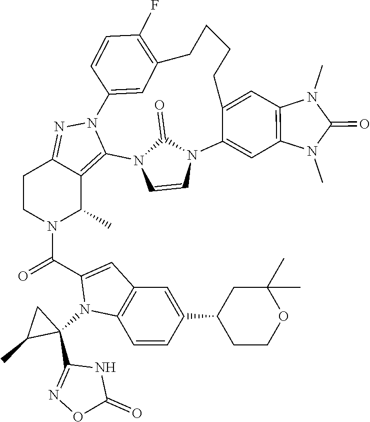

### Procedure for the Synthesis of Compound 103

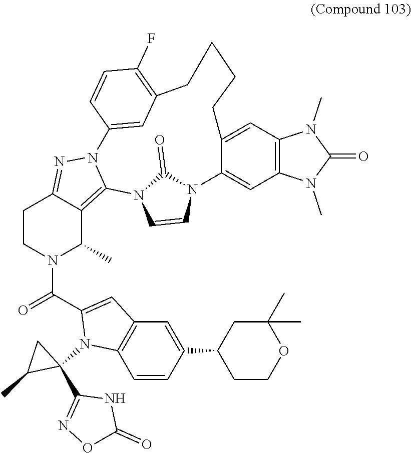

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-29-fluoro-8-methyl-18,20-dimethyl-2,3,7,11,14,18,20-heptaazaheptacyclo[26.3.1.1¹¹,¹⁴.0²,¹⁰.0⁴,⁹.0¹⁵,²³.0¹⁷,²¹]tritriaconta-1(32),3,9,12,15,17(21),22,28,30-nonaene-19,33-dione

Step A: A solution of 4,5-dibromo-1,2-benzenediamine (1 g, 3.76 mmol) and CDI (671 mg, 4.14 mmol) in dioxane (10 mL) was stirred at 60° C. over the weekend. The solution was concentrated, and the solids were washed with 1:4 ether:heptanes, then 0.5 M HCl, then dried and taken forward as the crude product. m/z (ESI-pos) [M+1]=290.9

Step B: A solution of 5,6-dibromo-1,3-dihydro-2H-1,3-benzimidazol-2-one (1.05 g, 3.6 mmol), K₂CO₃ (1.49 g, 10.8 mmol) and iodomethane (672 μL, 10.8 mmol) in DMF (10 mL) was stirred at room temperature overnight. The solution was then taken up in EtOAc and washed with water (3×). The organics were filtered through celite and concentrated. The solids were then triturated with 0.5M HCl and then 20% EtOAc/heptanes. The solids were dried overnight in the vacuum oven to give crude product. m/z (ESI-pos) [M+1]=319.0

Step C: A solution of tert-butyl (S)-3-amino-2-(3-bromo-4-fluorophenyl)-4-methyl-1,2,5-triaza-2,4,6,7-tetrahydro-5H-indene-5-carboxylate (295 mg, 694 μmol), XPhos Pd-G3 (58.8 mg, 69.4 μmol), and allyl Bpin (195 μL, 1.04 mmol) in 0.2 mL of 1M K₃PO₄ and 0.5 mL of dioxane was vacuum/backfilled with nitrogen and heated to 90° C. for 1 hour. The solution was then partitioned between DCM and water. The organics were dried with sodium sulfate, filtered, concentrated, and purified by silica gel chromatography (1-10% MeOH/dcm) to give tert-butyl (S)-2-(3-allyl-4-fluorophenyl)-3-amino-4-methyl-1,2,5-triaza-2,4,6,7-tetrahydro-5H-indene-5-carboxylate (245 mg, 634 μmol). m/z (ESI-pos) [M+1]=387.3

Step D: To a solution of tert-butyl (S)-2-(3-allyl-4-fluorophenyl)-3-amino-4-methyl-1,2,5-triaza-2,4,6,7-tetrahydro-5H-indene-5-carboxylate (290 mg, 750 μmol) and DIEA (393 μL, 2.25 mmol) in THF (3 mL) was added phenyl chloroformate (142 μL, 1.13 mmol) and stirred for 15 minutes. The solution was then concentrated and taken forward as the crude product. m/z (ESI-pos) [M+1]=507.3

Step E: A solution of tert-butyl (S)-2-(3-allyl-4-fluorophenyl)-4-methyl-3-[phenyl(oxycarbonylamino)]-1,2,5-triaza-2,4,6,7-tetrahydro-5H-indene-5-carboxylate (360 mg, 711 μmol), and dimethoxyethylamine (116 μL, 1.07 mmol) in pyridine (3 mL) was stirred for 1 hour. The solution was then concentrated and partitioned between 10% aq. CuSO₄ and DCM. The organics were dried with sodium sulfate, filtered, concentrated, and taken forward as the crude material. m/z (ESI-pos) [M+1]=518.4

Step F: A solution of tert-butyl (S)-2-(3-allyl-4-fluorophenyl)-3-[3-(2,2-dimethoxyethyl)ureido]-4-methyl-1,2,5-triaza-2,4,6,7-tetrahydro-5H-indene-5-carboxylate (360 mg, 696 μmol) and methanesulfonic acid (185 mg, 1.92 mmol) in THF (1 mL) was heated to 50° C. for 1 hour. The solution was then neutralized with sat. NaHCO₃ and then stirred with boc₂o (182 mg, 835 μmol) for 1 hour. The solution was then concentrated and purified by silica gel chromatography (1-10% MeOH/dcm) to give tert-butyl (S)-2-(3-allyl-4-fluorophenyl)-4-methyl-3-(2-oxo-2,3-dihydro-1H-imidazol-1-yl)-1,2,5-triaza-2,4,6,7-tetrahydro-5H-indene-5-carboxylate (170 mg, 375 μmol). m/z (ESI-pos) [M+1]=454.4

Step G: A solution of tert-butyl (S)-2-(3-allyl-4-fluorophenyl)-4-methyl-3-(2-oxo-2,3-dihydro-1H-imidazol-1-yl)-1,2,5-triaza-2,4,6,7-tetrahydro-5H-indene-5-carboxylate (0.1 g, 220 μmol), 5,6-dibromo-1,3-dimethyl-1,3-dihydro-2H-1,3-benzimidazol-2-one (106 mg, 331 μmol), Copper (I) iodide (21 mg, 110 μmol), (1S,2S)-1,2-bis(methylamino)cyclohexane (31.4 mg, 220 μmol), and K₂CO₃ (91.4 mg, 661 μmol) in NMP (1 mL) was vacuum/backfilled with nitrogen and heated to 110° C. overnight. The solution was then taken up in EtOAc and washed with water (3×). The organics were concentrated and purified by silica gel chromatography (1-10% MeOH/dcm) to give impure product which was taken forward into the Stille. m/z (ESI-pos) [M+1]=692.3

Step H: A solution of tert-butyl (S)-2-(3-allyl-4-fluorophenyl)-3-[3-(6-bromo-1,3-dimethyl-2-oxo-2,3-dihydro-1H-1,3-benzimidazol-5-yl)-2-oxo-2,3-dihydro-1H-imidazol-1-yl]-4-methyl-1,2,5-triaza-2,4,6,7-tetrahydro-5H-indene-5-carboxylate (80 mg, 116 μmol), allyltris(butyl)stannane (107 μL, 347 μmol), cataCXium A Pd G3 (16.8 mg, 23.1 μmol), and caesium fluoride (87.7 mg, 578 μmol) in dioxane (0.5 mL) was sparged with nitrogen and heated to 90° C. for 2 hours. The solution was then filtered through celite, concentrated, and purified by silica gel chromatography (10-95% EtOAc/heptanes) to give tert-butyl (S)-3-[3-(6-allyl-1,3-dimethyl-2-oxo-2,3-dihydro-1H-1,3-benzimidazol-5-yl)-2-oxo-2,3-dihydro-1H-imidazol-1-yl]-2-(3-allyl-4-fluorophenyl)-4-methyl-1,2,5-triaza-2,4,6,7-tetrahydro-5H-indene-5-carboxylate (62 mg, 47.4 μmol). m/z (ESI-pos) [M+1]=654.3

Step I: A solution of tert-butyl (S)-3-[3-(6-allyl-1,3-dimethyl-2-oxo-2,3-dihydro-1H-1,3-benzimidazol-5-yl)-2-oxo-2,3-dihydro-1H-imidazol-1-yl]-2-(3-allyl-4-fluorophenyl)-4-methyl-1,2,5-triaza-2,4,6,7-tetrahydro-5H-indene-5-carboxylate (180 mg, 275 μmol) and Hoveyda-Grubbs II (86.3 mg, 138 μmol) and acetic acid (15.7 μL, 275 μmol) in DCE (45 mL) was sparged with nitrogen for 5 minutes and heated to 60° C. for 1 hour. The solution was concentrated and purified by silica gel chromatography (10-95% EtOAc/dcm) to give tert-butyl (S)-29-fluoro-8-methyl-18,20-dimethyl-19,33-dioxo-2,3,7,11,14,18,20-heptaazaheptacyclo[26.3.1.1¹¹,¹⁴.0²,¹⁰.0⁴,⁹.0¹⁵,²³.0¹⁷,²¹]tritriaconta 1(32),3,9,12,15,17(21),22,25,28,30-decaene-7-carboxylate (130 mg, 208 μmol). m/z (ESI-pos) [M+1]=626.4

Step J: A solution of tert-butyl (S)-29-fluoro-8-methyl-18,20-dimethyl-19,33-dioxo-2,3,7,11,14,18,20-heptaazaheptacyclo[26.3.1.1¹¹,¹⁴.0²,¹⁰.0⁴,⁹.0¹⁵,²³.0¹⁷,²¹]tritriaconta-1(32),3,9,12,15,17(21),22,25,28,30-decaene-7-carboxylate (115 mg, 184 μmol) and Pd/C 10% w/w (19.6 mg, 18.4 μmol) in MeOH (2 mL) was sparged with hydrogen and then sealed and stirred for 1 hour. The solution was filtered through celite, concentrated, and stirred in 1 mL of 4M HCl in dioxane for 1 hour. The solution was concentrated and taken forward as the crude HCl salt. m/z (ESI-pos) [M+1]=528.4

Step K: A solution of (S)-29-fluoro-8-methyl-18,20-dimethyl-2,3,7,11,14,18,20-heptaazaheptacyclo[26.3.1.1¹¹,¹⁴.0²,¹⁰.0⁴,⁹.0¹⁵,²³.0¹⁷,²¹]tritriaconta-1(32),3,9,12,15,17(21),22,28,30-nonaene-19,33-dione-hydrogen chloride (1/1) (95 mg, 168 μmol), 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (90.1 mg, 219 μmol), HATU (96.1 mg, 253 μmol), and DIEA (88 μL, 505 μmol) in DMF (1 mL) was stirred for 1 hour at 50° C. The solution was then taken up in EtOAc and washed with water (3×). The organics were concentrated and purified by silica gel chromatography (1-10% MeOH/DCM) to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-29-fluoro-8-methyl-18,20-dimethyl-2,3,7,11,14,18,20-heptaazaheptacyclo[26.3.1.1¹¹,¹⁴.0²,¹⁰.0⁴,⁹.0¹⁵,²³.0¹⁷,²¹]tritriaconta-1(32),3,9,12,15,17(21),22,28,30-nonaene-19,33-dione (52 mg, 54.8 μmol). m/z (ESI-pos) [M+1]=921.7; ¹H NMR (400 MHz, chloroform-D) δ 11.26 (d, J=22.5 Hz, 1H), 7.57 (t, J=8.6 Hz, 1H), 7.51 (s, 1H), 7.46 (dt, J=8.7, 3.2 Hz, 1H), 7.23-7.06 (m, 2H), 6.90-6.73 (m, 3H), 6.72-6.66 (m, 1H), 6.42 (dd, J=3.1, 1.9 Hz, 1H), 6.33-6.21 (m, 1H), 5.88-5.75 (m, 1H), 4.45 (dd, J=13.6, 5.1 Hz, 1H), 3.84 (d, J=11.8 Hz, 3H), 3.76-3.56 (m, 1H), 3.48 (d, J=5.5 Hz, 4H), 3.45-3.40 (m, 7H), 3.39-3.28 (m, 1H), 3.23-3.10 (m, 1H), 3.02 (d, J=12.8 Hz, 3H), 2.64 (t, J=11.8 Hz, 1H), 1.90 (t, J=5.6 Hz, 1H), 1.88-1.71 (m, 4H), 1.66 (dd, J=12.6, 7.0 Hz, 3H), 1.56 (d, J=18.2 Hz, 4H), 1.50-1.46 (m, 3H), 1.43 (d, J=6.6 Hz, 3H), 1.33 (d, J=2.4 Hz, 6H), 1.30-1.23 (m, 6H), 1.19 (d, J=5.6 Hz, 3H), 1.07 (dd, J=31.7, 5.6 Hz, 1H), 0.90 (d, J=5.8 Hz, 1H).

### Example 104—Synthesis of Compound 104

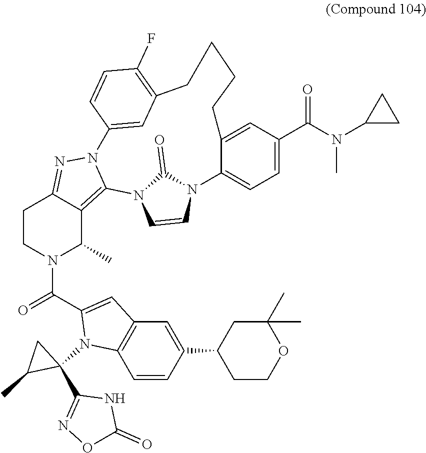

### N-cyclopropyl-N-methyl-(S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-18-carboxamide

Compound 104 was synthesized following the procedure of Example 103, with(S)-29-fluoro-8-methyl-18,20-dimethyl-2,3,7,11,14,18,20-heptaazaheptacyclo[26.3.1.1¹¹,¹⁴.0²,¹⁰.0⁴,⁹.0¹⁵,²³.0¹⁷,²¹]tritriaconta-1(32),3,9,12,15,17(21),22,28,30-nonaene-19,33-dione replaced by N-cyclopropyl-N-methyl-(S)-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-18-carboxamide. m/z (ESI-pos) [M+1]=934.7; ¹H NMR (400 MHz, chloroform-D) δ 11.25 (s, 1H), 7.66-7.39 (m, 6H), 7.15 (t, J=9.6 Hz, 2H), 6.75-6.08 (m, 3H), 5.91-5.67 (m, 1H), 5.04-4.87 (m, 2H), 4.46 (s, 1H), 4.16 (s, 1H), 3.85 (s, 3H), 3.08 (d, J=17.4 Hz, 6H), 2.85 (d, J=23.7 Hz, 2H), 2.60 (d, J=24.7 Hz, 2H), 2.47-2.35 (m, 1H), 2.08-1.92 (m, 4H), 1.76 (m, 5H), 1.63 (s, 6H), 1.20-1.13 (m, 6H), 0.85-0.47 (m, 4H).

### Example 105—Synthesis of Compound 105

### Synthetic Scheme

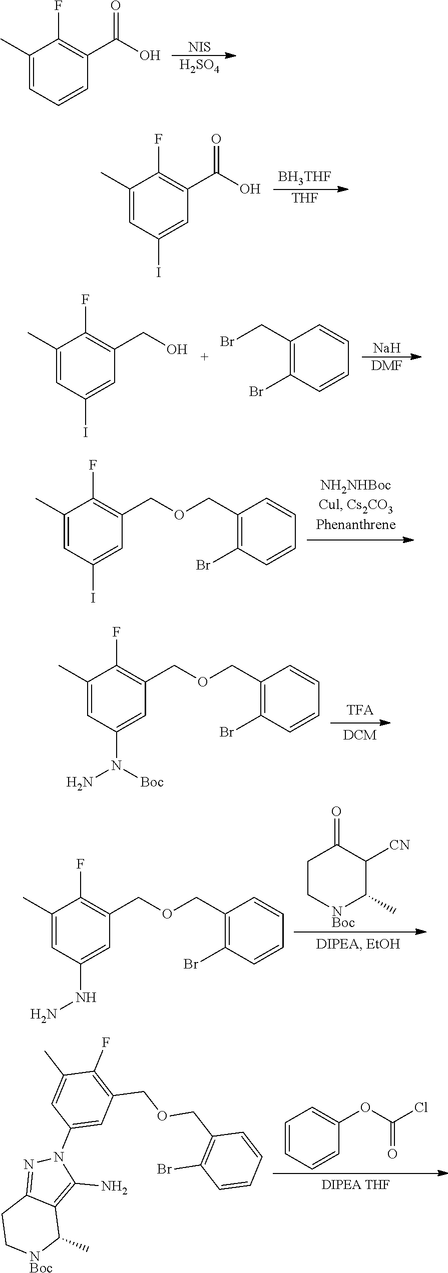

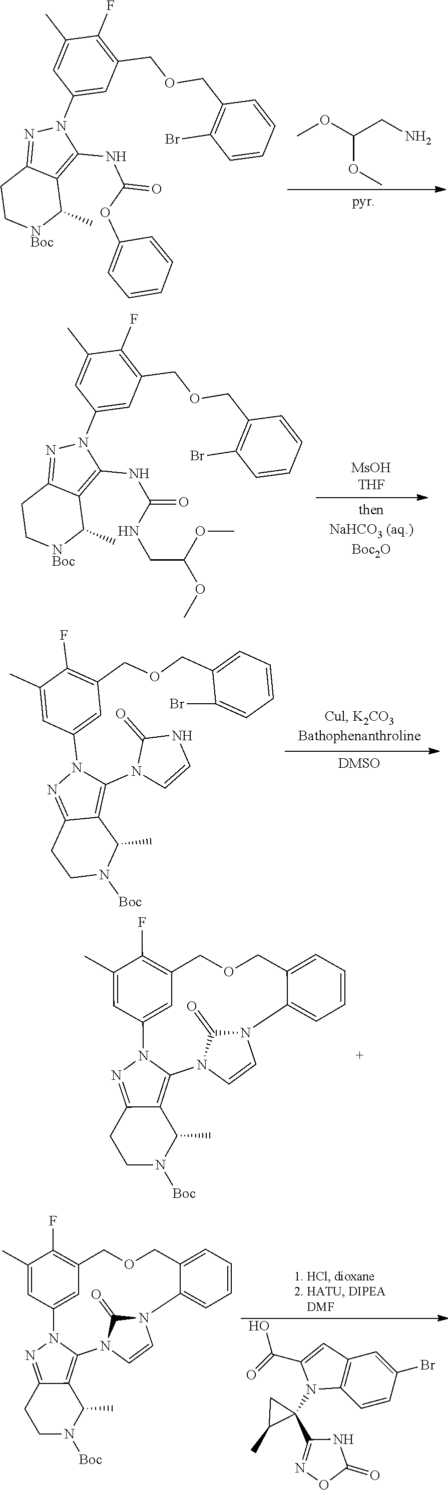

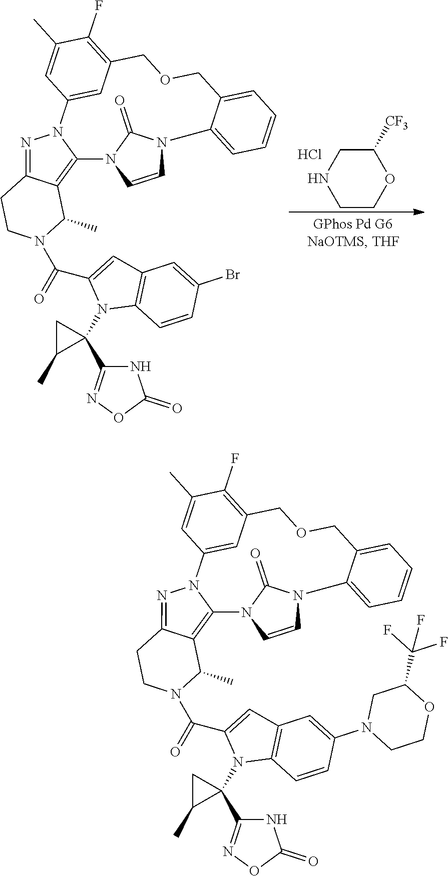

### Procedure for Synthesis of Compound 105

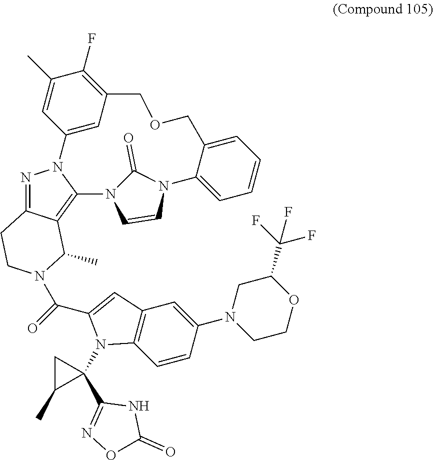

### (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Step A: 2-fluoro-3-toluic acid (15 g, 97.3 mmol) and conc. aqueous sulfuric acid (75 mL) were added into the reaction flask and cooled to 0° C., 1-iodo-2,5-pyrrolidinedione (21.9 g, 97.3 mmol) was added portion-wise. After the addition was complete, the reaction temperature was naturally raised to room temperature and stirred for 2h. Upon completion, the reaction was cooled in an ice bath and diluted with water, the mixture was filtered, rinsing with aqueous sodium dithionate solution (~500 mg in 200 mL water), then twice with water. The filter cake was dried in a vacuum oven overnight to afford 2-fluoro-5-iodo-3-toluic acid (26.1 g, 93.1 mmol). 1H NMR (400 MHz, CHLOROFORM-D) δ 11.41 (s, 1H), 8.12 (dd, J=6.3, 2.4 Hz, 1H), 7.73 (ddd, J=6.3, 2.4, 0.9 Hz, 1H), 2.28 (d, J=2.4 Hz, 3H).

Step B: 1M Borane in THF (12.9 g, 150 mmol) was added dropwise to a stirred solution of 2-fluoro-5-iodo-3-toluic acid (21 g, 75 mmol) in THF (75 mL) cooled to 0° C. The resulting mixture was warmed to room temperature and stirred for an additional 3 h. The mixture was poured into a sat. aq. ammonium chloride solution and extracted with ethyl acetate. The combined organic phases were washed with sat. aq. sodium bicarbonate, brine, dried over Na₂SO₄ and concentrated to afford (2-fluoro-5-iodo-3-tolyl)methanol (19.9 g, 75 mmol) which was used in the next step without purification. 1H NMR (400 MHz, CHLOROFORM-D) δ 7.56 (ddq, J=6.4, 2.5, 0.7 Hz, 1H), 7.48-7.41 (m, 1H), 4.69 (d, J=4.5 Hz, 2H), 2.22 (dt, J=2.1, 0.7 Hz, 3H), 1.74 (d, J=6.3 Hz, 1H).

Step C: To a solution of (2-fluoro-5-iodo-3-tolyl)methanol (970 mg, 3.65 mmol) and o-bromo (bromomethyl)benzene (911 mg, 3.65 mmol) in DMF (7.29 mL) was added sodium hydride 60% w/w (153 mg, 3.83 mmol) at 0° C. The reaction was stirred at 0° C. for 30 min and then at ambient temperature for 30 min. The reaction mixture was quenched with sat. aq. NH₄Cl, diluted with ethyl acetate, and washed with brine (2×). The organic layer was dried over Na₂SO₄, filtered and concentrated. The crude product was purified by silica gel column chromatography (0-10% EtOAc/heptane) to give 3-{[(o-bromophenyl)methoxy]methyl}-2-fluoro-5-iodotoluene (1.04 g, 2.4 mmol) as a yellow oil.

Step D: A solution of 3-{[(o-bromophenyl)methoxy]methyl}-2-fluoro-5-iodotoluene (1.04 g, 2.4 mmol), tert-butyl carbazate (413 mg, 3.12 mmol), dicaesium carbonate (1.1 g, 3.36 mmol), copper iodide (22.9 mg, 120 μmol), and 4,5-diazaphenanthrene (86.6 mg, 480 μmol) in DMF (2.4 mL) was sparged with Ar for 5 min and heated to 80° C. for 3h. Upon cooling to rt, the solution was taken up in EtOAc, filtered through Celite, and washed with brine (3×). The organic phase was dried over Na₂SO₄, filtered, and concentrated. The crude product was purified by silica gel chromatography (5-30% EtOAc/heptane) to give tert-butyl 2-(m-{[2-(2-bromo-4,5-difluorophenyl)ethoxy]methyl}phenyl)carbazate (347 mg, 759 μmol) as a yellow oil. m/z (ESI-pos) M+1=383.1 and 385.1 (loss of tBu).

Step E: To a solution of tert-butyl 2-(3-{[(o-bromophenyl)methoxy]methyl}-4-fluoro-5-tolyl)carbazate (0.8 g, 1.82 mmol) in DCM (6.07 mL), trifluoroacetic acid (2.09 mL, 27.3 mmol) was added at ambient temperature. The reaction was stirred for 3 h. The reaction was then quenched with sat. aq. Na₂CO₃ (~20 mL) and extracted with DCM (1×), then washed with water (2×) and brine (1×). The combined aqueous layers were then extracted with 20% IPA/CHCl₃ (2×) and the combined organic layers were dried over Na₂SO₄, filtered, and concentrated to provide (3-{[(o-bromophenyl)methoxy]methyl}-4-fluoro-5-tolyl) hydrazine (618 mg, 1.82 mmol) as a yellow oil which was used without purification in the following step. m/z (ESI-pos) M+1=338.1 and 340.1.

Step F: To a solution of crude (3-{[(o-bromophenyl)methoxy]methyl}-4-fluoro-5-tolyl) hydrazine (618 mg, 1.82 mmol) in EtOH (7.29 mL) was added N-ethyldiisopropylamine (317 μL, 1.82 mmol) then tert-butyl(2S)-3-cyano-2-methyl-4-oxo-1-piperidinecarboxylate (521 mg, 2.19 mmol) at ambient temperature. The mixture was then stirred at 80° C. for 20 h. After completion, the reaction mixture was cooled, and the solvent was evaporated under vacuo. The crude material was purified by column chromatography on silica gel (10-60% EtOAc/heptane) to give tert-butyl (S)-3-amino-2-(3-{[(o-bromophenyl)methoxy]methyl}-4-fluoro-5-tolyl)-4-methyl-1,2,5-triaza-2,4,6,7-tetrahydro-5H-indene-5-carboxylate (554 mg, 990 μmol) as a yellow solid over 2 steps. m/z (ESI-pos) M+1=559.3 and 561.3.

Step G: To a solution of tert-butyl (S)-3-amino-2-(3-{[(o-bromophenyl)methoxy]methyl}-4-fluoro-5-tolyl)-4-methyl-1,2,5-triaza-2,4,6,7-tetrahydro-5H-indene-5-carboxylate (554 mg, 990 μmol) in THF (5 mL) was added N-ethyldiisopropylamine (517 μL, 2.97 mmol) followed by benzenyl chloroformate (248 μL, 1.98 mmol). The reaction mixture was stirred at room temperature for 30 min. The reaction mixture was directly concentrated under vacuo to give the crude tert-butyl (S)-2-(3-{[(o-bromophenyl)methoxy]methyl}-4-fluoro-5-tolyl)-4-methyl-3-[phenyl(oxycarbonylamino)]-1,2,5-triaza-2,4,6,7-tetrahydro-5H-indene-5-carboxylate (673 mg, 990 μmol) as a yellow solid, which was used directly in the next step without any further purification. m/z (ESI-pos) M+1=679.3 and 681.3.

Step H: Crude tert-butyl (S)-2-(3-{[(o-bromophenyl)methoxy]methyl}-4-fluoro-5-tolyl)-4-methyl-3-[phenyl(oxycarbonylamino)]-1,2,5-triaza-2,4,6,7-tetrahydro-5H-indene-5-carboxylate (673 mg, 990 μmol) was dissolved in pyridine (3.3 mL) and then 2,2-dimethoxyethylamine (162 μL, 1.49 mmol) was added. The mixture was stirred at room temperature for 30 min and then directly concentrated under vacuo to give the crude residue which was purified by column chromatography (5-80% EtOAc/heptane) to give tert-butyl (S)-2-(3-{[(o-bromophenyl)methoxy]methyl}-4-fluoro-5-tolyl)-3-[3-(2,2-dimethoxyethyl)ureido]-4-methyl-1,2,5-triaza-2,4,6,7-tetrahydro-5H-indene-5-carboxylate (464 mg, 672 μmol) as a white solid over 2 steps. m/z (ESI-pos) M+1=689.3 and 691.3.

Step I: To a solution of tert-butyl (S)-2-(3-{[(2-bromo-4-fluorophenyl)methoxy]methyl}-4-fluoro-5-tolyl)-3-[3-(2,2-dimethoxyethyl)ureido]-4-methyl-1,2,5-triaza-2,4,6,7-tetrahydro-5H-indene-5-carboxylate (464 mg, 655 μmol) in THF (3.3 mL) was added methanesulfonic acid (128 μL, 1.96 mmol). The mixture was stirred at 55° C. for 1 h and then cooled to ambient temperature. Next, saturated aqueous NaHCO₃ was added until the solution was pH 8 and then tert-butoxycarbonyl-tert-butyl carbonate (157 mg, 720 μmol) was added, and the resulting mixture was stirred at room temperature for 30 min. Upon completion, the reaction mixture was diluted with water and extracted with ethyl acetate (2×). The combined organic layers were washed with brine and dried over anhydrous Na₂SO₄, filtered and concentrated under vacuo. The crude compound was purified by column chromatography (5-90% EtOAc/heptane) to give tert-butyl (S)-2-(3-{[(o-bromophenyl)methoxy]methyl}-4-fluoro-5-tolyl)-4-methyl-3-(2-oxo-2,3-dihydro-1H-imidazol-1-yl)-1,2,5-triaza-2,4,6,7-tetrahydro-5H-indene-5-carboxylate (340 mg, 543 μmol) as an off-white solid. m/z (ESI-pos) M+1=626.3 and 628.3.

Step J: A solution of tert-butyl (S)-2-(3-{[(o-bromophenyl)methoxy]methyl}-4-fluoro-5-tolyl)-4-methyl-3-(2-oxo-2,3-dihydro-1H-imidazol-1-yl)-1,2,5-triaza-2,4,6,7-tetrahydro-5H-indene-5-carboxylate (340 mg, 543 μmol), copper iodide (77.5 mg, 407 μmol), bathophenanthroline (180 mg, 543 μmol), and dipotassium carbonate (187 mg, 1.36 mmol) in DMSO (18.1 mL) was sparged with Ar for 5 minutes and then heated to 130° C. overnight for 24 h. The reaction was diluted with EtOAc and sat. aq. NH₄Cl and then filtered through a pad of celite. The filtrate was partitioned, and the organic phase was washed with brine, dried with sodium sulfate, filtered, concentrated under vacuo. The crude material was purified by silica gel chromatography (5-50% EtOAc/heptane), which provided separation of the isomers. The major isomer was tert-butyl (8S,10M)-25-fluoro-8,26-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (100 mg, 183 μmol). m/z (ESI-pos) M+1=546.4.

Step K: 4.0M HCl in dioxane (2 mL) was added to a vial containing tert-butyl (8S,10M)-25-fluoro-8,26-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (27.1 mg, 49.7 μmol) and the solution stirred at ambient temperature for 20 min. Upon completion, the solvent was evaporated to give a crude white residue which was used in the next step without purification. m/z (ESI-pos) M+1=446.3

Step L: To a solution of crude (8S,10M)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (500 mg, 1.04 mmol) and 5-bromo-1-[(1S,2S)-2-methyl-1-(5-oxo-4,5-dihydro-1,2,4-oxadiazol-3-yl)cyclopropyl]-2-indolecarboxylic acid (432 mg, 1.14 mmol) in DMF (3.5 mL) was added N-ethyldiisopropylamine (723 μL, 4.15 mmol) and HATU (552 mg, 1.45 mmol). The mixture stirred at ambient temperature overnight. The reaction mixture was directly loaded onto the reverse phase column and purified by Prep HPLC (Isco ACCQ Prep, Gemini column NX-C18, 250×21 mm, 20 ml/min, 45 minute separation, 20 to 80% H₂O/ACN with 0.1% Formic acid buffer), pure fractions were collected and lyophilized to give (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (710 mg, 881 μmol). m/z (ESI-pos) M+2=807.4.

Step M: To a mixture of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (10 mg, 12.4 μmol), (R)-2-(trifluoromethyl)morpholine hydrochloride (7.1 mg, 37.2 mmol), bromidopalladium(1+) 2-(trimethylsilyl)ethyl 1-benzenid-4-carboxylate-(3-tert-butoxy-2′,6′-diisopropyl-6-methoxy-2-biphenylyl)dicyclohexylphosphine (1/1) (1.76 mg, 1.86 μmol), and sodium trimethylsilanolate (5.57 mg, 49.6 μmol), was added tetrahydrofuran (0.5 mL). The mixture was argon sparged for 5 minutes then heated to 70° C. for 16 hours. The crude residue was purified by reverse-phase prep-HPLC purification eluting with 20-75% MeCN+0.1% FA in water+0.1% FA. Desired fractions were collected and lyophilized to give (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (4 mg, 4.55 μmol). m/z (ESI-pos) M+1=880.6; ¹H NMR (400 MHz, CHLOROFORM-D) δ 11.32 (s, 1H), 7.59-7.53 (m, 2H), 7.49-7.44 (m, 4H), 7.15-7.10 (m, 3H), 6.66 (s, 1H), 6.43 (d, J=3.1 Hz, 1H), 6.28 (d, J=3.0 Hz, 1H), 5.79 (q, J=6.7 Hz, 1H), 4.79-4.68 (m, 2H), 4.49-4.43 (m, 1H), 4.32-4.28 (m, 1H), 4.17-4.14 (m, 3H), 3.94-3.87 (m, 1H), 3.66-3.51 (m, 2H), 3.39-3.34 (m, 1H), 3.16-2.85 (m, 4H), 2.32 (s, 3H), 1.90 (t, J=6.2 Hz, 1H), 1.66 (d, J=6.7 Hz, 3H), 1.52-1.49 (m, 2H), 1.20 (d, J=6.0 Hz, 3H).

### Procedure for Synthesis of Compound 106

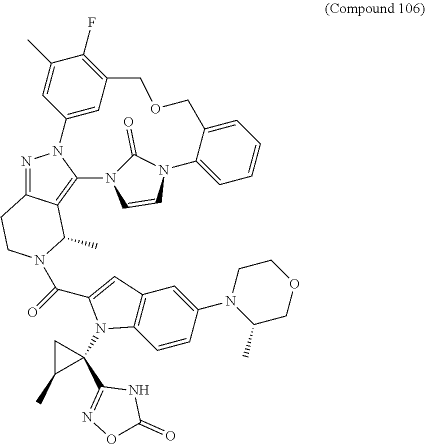

### (8S,10M)-25-fluoro-8,26-dimethyl-7-({5-[(S)-3-methyl-4-morpholinyl]-1-[(1S,2S)-2-methyl-1-(5-oxo-4,5-dihydro-1,2,4-oxadiazol-3-yl)cyclopropyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 106 was synthesized following the procedure of Example 105, (R)-2-(trifluoromethyl)morpholine being replaced with(S)-3-methylmorpholine in step M. m/z (ESI-pos) M+1=826.6; 1H NMR (400 MHz, CHLOROFORM-D) δ 11.33 (s, 1H), 7.54-7.44 (m, 6H), 7.20-7.09 (m, 3H), 6.64 (s, 1H), 6.43 (d, J=3.1 Hz, 1H), 6.28 (d, J=3.1 Hz, 1H), 5.79 (q, J=6.6 Hz, 1H), 4.82-4.65 (m, 2H), 4.54-4.11 (m, 3H), 3.93-3.51 (m, 6H), 3.17-3.00 (m, 4H), 2.33 (s, 3H), 1.89 (t, J=5.7 Hz, 1H), 1.66 (d, J=6.7 Hz, 3H), 1.53-1.50 (m, 2H), 1.20 (d, J=5.8 Hz, 3H), 0.96 (d, J=6.4 Hz, 3H).

### Procedure for Synthesis of Compound 107

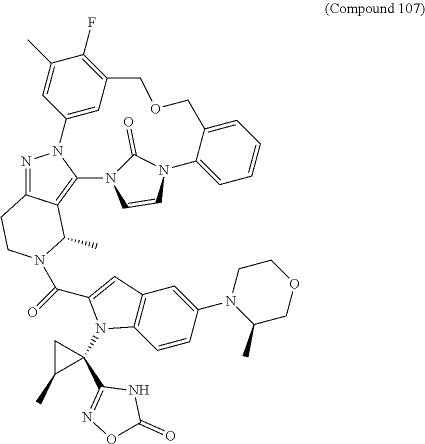

### (8S,10M)-25-fluoro-8,26-dimethyl-7-({5-[(R)-3-methyl-4-morpholinyl]-1-[(1S,2S)-2-methyl-1-(5-oxo-4,5-dihydro-1,2,4-oxadiazol-3-yl)cyclopropyl]-2-indolyl}carbonyl)-22-oxa-2,3,7,11,14-pentaazahexacyclo[23.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 107 was synthesized following the procedure of Example 105, (R)-2-(trifluoromethyl)morpholine being replaced with (R)-3-methylmorpholine in step M. m/z (ESI-pos) M+1=826.6; 1H NMR (400 MHz, CHLOROFORM-D) δ 11.31 (s, 1H), 7.56-7.44 (m, 7H), 7.20-7.09 (m, 2H), 6.64 (s, 1H), 6.44 (d, J=3.1 Hz, 1H), 6.28 (d, J=3.0 Hz, 1H), 5.79 (q, J=6.7 Hz, 1H), 4.95-4.46 (m, 2H), 4.33-4.12 (m, 2H), 3.94-3.79 (m, 3H), 3.67-3.45 (m, 3H), 3.18-2.97 (m, 5H), 2.33 (s, 3H), 1.89 (t, J=5.5 Hz, 1H), 1.66 (d, J=6.7 Hz, 3H), 1.63-1.53 (m, 2H) 1.20 (d, J=5.7 Hz, 3H), 0.95 (d, J=6.4 Hz, 3H).

### Example 108—Synthesis of Compound 108

### Synthetic Scheme

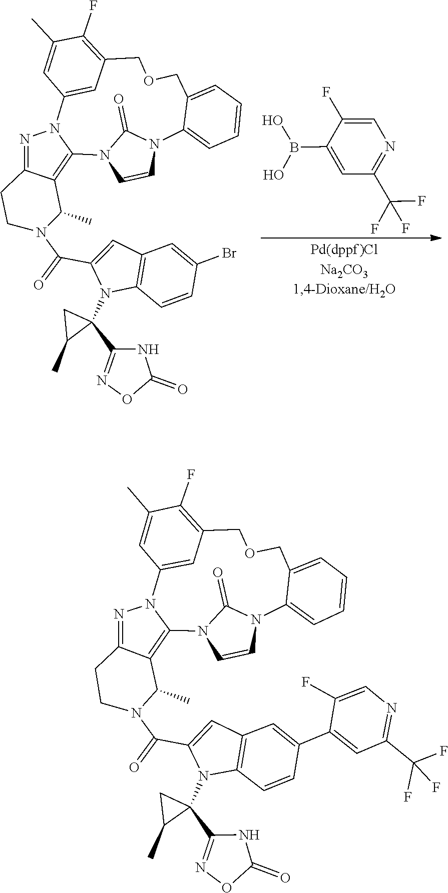

### Procedure for Synthesis of Compound 108

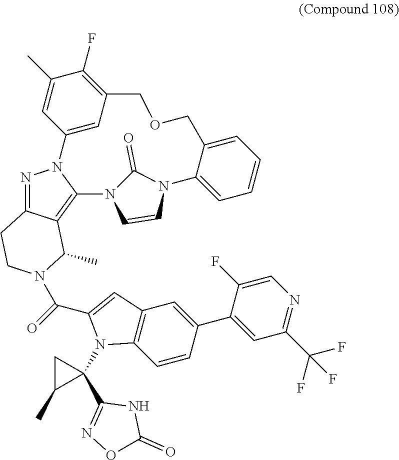

### (8S,10M)-25-fluoro-7-({5-[5-fluoro-2-(trifluoromethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (20 mg, 24.8 μmol), [5-fluoro-2-(trifluoromethyl)-4-pyridyl]boranediol (25.9 mg, 124 μmol) and Na₂CO₃ (13.2 mg, 124 μmol) in 1,4-dioxane (0.6 mL) and water (0.2 mL) was added Pd(dppf)Cl₂ dichloromethane complex (4 mg, 4.96 μmol). The mixture was stirred at 80° C. for 1 h and then the reaction mixture was cooled to ambient temperature. The solution was filtered and directly and then purified by reverse-phase chromatography (25-45% MeCN/water with 0.1% FA) and the product was lyophilized to afford (8S,10M)-25-fluoro-7-({5-[5-fluoro-2-(trifluoromethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (14 mg, 15.7 μmol). m/z (ESI-pos) M+1=890.5; 1H NMR (400 MHz, CHLOROFORM-D) δ 11.24 (s, 1H), 8.63 (s, 1H), 7.97 (s, 1H), 7.86-7.76 (m, 2H), 7.64-7.46 (m, 6H), 7.13-7.09 (m, 1H), 6.83 (s, 1H), 6.44 (d, J=3.0 Hz, 1H), 6.28 (d, J=3.0 Hz, 1H), 5.83 (q, J=6.6 Hz, 1H), 4.80-4.67 (m, 2H), 4.50-4.12 (m, 3H), 3.72-3.63 (m, 1H), 3.21-3.03 (m, 2H), 2.35-2.32 (m, 3H), 1.98 (t, J=6.1 Hz, 1H), 1.69 (d, J=6.7 Hz, 3H), 1.64-1.61 (m, 2H), 1.23 (d, J=6.0 Hz, 3H).

### Procedure for Synthesis of Compound 109

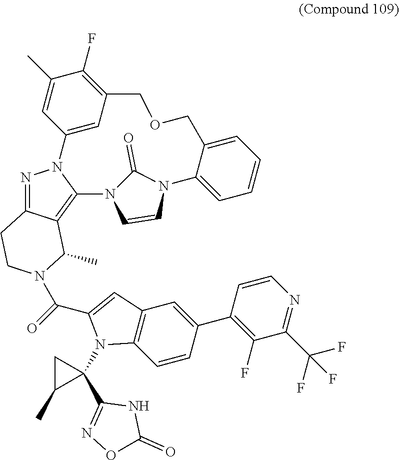

### (8S,10M)-25-fluoro-7-({5-[5-fluoro-2-(trifluoromethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 109 was synthesized following the procedure of Example 108, with [5-fluoro-2-(trifluoromethyl)-4-pyridyl]boranediol being replaced with 2-[3-fluoro-2-(trifluoromethyl)-4-pyridyl]. m/z (ESI-pos) M+1=890.5; 1H NMR (400 MHz, CHLOROFORM-D) δ 11.26 (s, 1H), 8.54 (d, J=4.9 Hz, 1H), 7.99-7.94 (m, 1H), 7.81-7.75 (m, 1H), 7.66-7.63 (m, 1H), 7.60-7.54 (m, 2H), 7.51-7.43 (m, 4H), 7.11-7.09 (m, 1H), 6.82 (s, 1H), 6.45 (d, J=3.0 Hz, 1H), 6.29 (d, J=3.0 Hz, 1H), 5.83 (q, J=6.6 Hz, 1H), 4.96-4.65 (m, 2H), 4.51-4.11 (m, 3H), 3.72-3.63 (m, 1H), 3.20-3.05 (m, 2H), 2.34 (s, 3H), 1.98 (t, J=6.0 Hz, 1H), 1.69 (d, J=6.7 Hz, 3H), 1.61-1.55 (m, 2H), 1.24 (d, J=5.9 Hz, 3H).

### Procedure for Synthesis of Compound 110

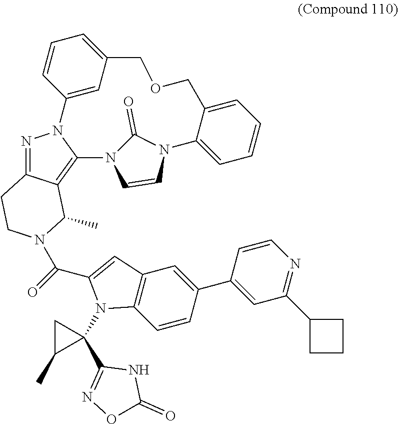

### (8S,10M)-7-{[5-(2-cyclobutyl-4-pyridyl)-1-[(1S,2S)-2-methyl-1-(5-oxo-4,5-dihydro-1,2,4-oxadiazol-3-yl)cyclopropyl]-2-indolyl]carbonyl}-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 110 was synthesized following the procedure of Examples 90 and 108, with [5-fluoro-2-(trifluoromethyl)-4-pyridyl]boranediol being replaced with (2-cyclobutyl-4-pyridyl)boranediol. m/z (ESI-pos) M+1=826.6; 1H NMR (400 MHz, CHLOROFORM-D) δ 11.30 (s, 1H), 8.61 (d, J=5.2 Hz, 1H), 7.95-7.93 (m, 1H), 7.77-7.732 (m, 1H), 7.67-7.62 (m, 2H), 7.54-7.40 (m, 7H), 7.37-7.34 (m, 1H), 7.22-7.17 (m, 1H), 6.82 (s, 1H), 6.45 (d, J=3.0 Hz, 1H), 6.30 (d, J=3.0 Hz, 1H), 5.84 (q, J=6.6 Hz, 1H), 4.67 (s, 2H), 4.48 (dd, J=13.8, 5.0 Hz, 1H), 4.30 (d, J=9.3 Hz, 1H), 4.10 (d, J=9.3 Hz, 1H), 3.81-3.63 (m, 2H), 3.23-3.03 (m, 2H), 2.44-2.37 (m, 4H), 2.15-2.06 (m, 1H), 2.01-1.93 (m, 2H), 1.68 (d, J=6.7 Hz, 3H), 1.63-1.54 (m, 2H), 1.23 (d, J=6.0 Hz, 3H).

### Procedure for Synthesis of Compound 111

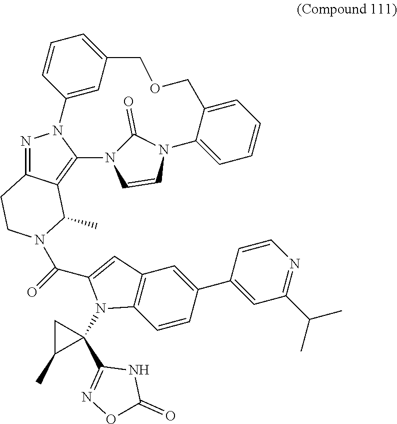

### (8S,10M)-7-{[5-(2-isopropyl-4-pyridyl)-1-[(1S,2S)-2-methyl-1-(5-oxo-4,5-dihydro-1,2,4-oxadiazol-3-yl)cyclopropyl]-2-indolyl]carbonyl}-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 111 was synthesized following the procedure of Examples 90 and 108, with [5-fluoro-2-(trifluoromethyl)-4-pyridyl]boranediol being replaced with 2-(2-isopropyl-4-pyridyl)-4,4,5,5-tetramethyl-1,3,2-dioxaborolane. m/z (ESI-pos) M+1=814.6; 1H NMR (400 MHz, CHLOROFORM-D) δ 11.30 (s, 1H), 8.58 (d, J=5.1 Hz, 1H), 7.94 (s, 1H), 7.75 (d, J=8.6 Hz, 1H), 7.64 (dd, J=8.5, 1.9 Hz, 2H), 7.54-7.39 (m, 7H), 7.35 (dd, J=5.2, 1.8 Hz, 1H), 7.22-7.17 (m, 1H), 6.82 (s, 1H), 6.45 (d, J=3.0 Hz, 1H), 6.30 (d, J=3.1 Hz, 1H), 5.90-5.78 (m, 1H), 4.67 (s, 2H), 4.58-4.43 (m, 1H), 4.27 (d, J=9.3 Hz, 1H), 4.10 (d, J=9.3 Hz, 1H), 3.68 (td, J=13.1, 4.0 Hz, 1H), 3.23-3.03 (m, 3H), 1.97 (t, J=6.0 Hz, 1H), 1.69 (d, J=6.7 Hz, 3H), 1.65-1.59 (m, 2H), 1.37 (d, J=6.9 Hz, 6H), 1.23 (d, J=6.0 Hz, 3H).

### Procedure for Synthesis of Compound 112

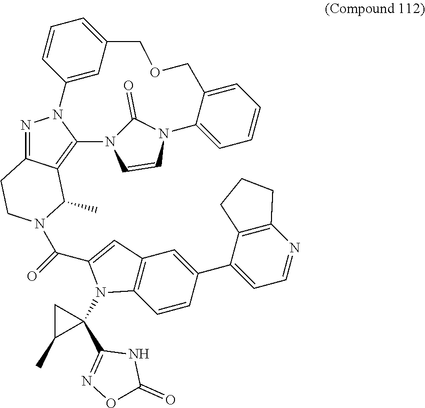

### (8S,10M)-7-{[5-(4-aza-7-indanyl)-1-[(1S,2S)-2-methyl-1-(5-oxo-4,5-dihydro-1,2,4-oxadiazol-3-yl)cyclopropyl]-2-indolyl]carbonyl}-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 112 was synthesized following the procedure of Examples 90 and 108, with [5-fluoro-2-(trifluoromethyl)-4-pyridyl]boranediol being replaced with (4-aza-7-indanyl)boranediol. m/z (ESI-pos) M+1=812.6; 1H NMR (400 MHz, CHLOROFORM-D) δ 11.31 (s, 1H), 8.40 (d, J=5.2 Hz, 1H), 7.84-7.69 (m, 2H), 7.67-7.60 (m, 1H), 7.53-7.43 (m, 7H), 7.21 (d, J=7.7 Hz, 1H), 7.12 (d, J=5.2 Hz, 1H), 6.79 (s, 1H), 6.45 (d, J=3.0 Hz, 1H), 6.30 (d, J=3.0 Hz, 1H), 5.84 (q, J=6.6 Hz, 1H), 4.67 (s, 2H), 4.49 (dd, J=13.8, 5.0 Hz, 1H), 4.30 (d, J=9.4 Hz, 1H), 4.13-4.04 (m, 1H), 3.67 (td, J=13.1, 4.1 Hz, 1H), 3.19-3.05 (m, 6H), 2.15-2.09 (m, 2H), 1.96 (t, J=5.7 Hz, 1H), 1.69 (d, J=6.7 Hz, 3H), 1.62-1.57 (m, 2H), 1.23 (d, J=5.8 Hz, 3H).

### Example 113—Synthesis of Compound 113

### Synthetic Scheme

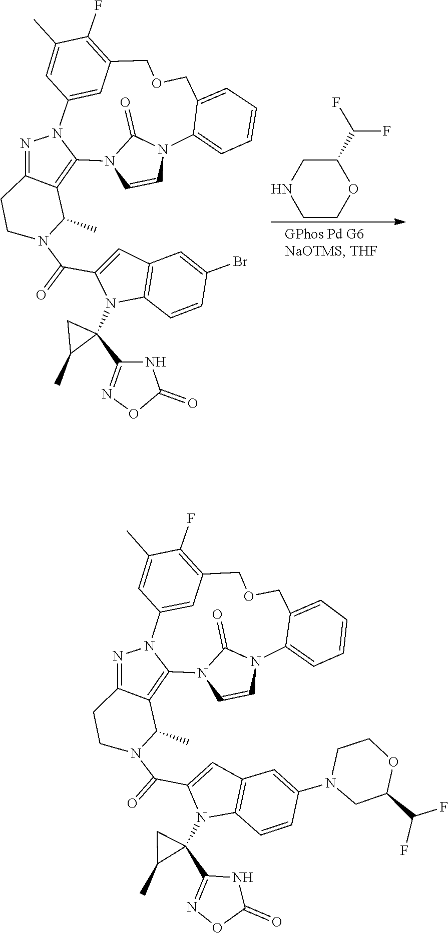

### Procedure for Synthesis of Compound 113

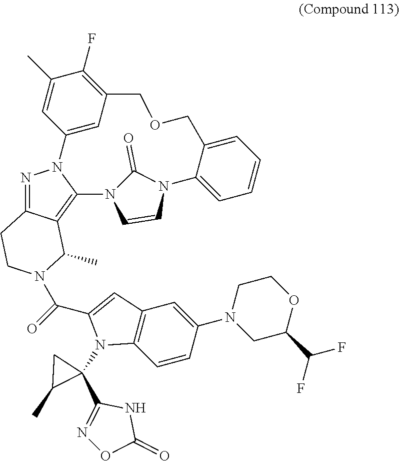

### (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Step A: To a mixture of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (25 mg, 31 μmol), (R)-2-(difluoromethyl)morpholine (17 mg, 124 μmol), bromidopalladium(1+) 2-(trimethylsilyl)ethyl 1-benzenid-4-carboxylate-(3-tert-butoxy-2′,6′-diisopropyl-6-methoxy-2-biphenylyl)dicyclohexylphosphine (1/1) (4.4 mg, 4.65 μmol), and sodium trimethylsilanolate (24.4 mg, 217 μmol), was added tetrahydrofuran (0.8 mL). The mixture was argon sparged for 5 minutes then heated to 70° C. for 16 hours. The crude residue was purified by reverse-phase prep-HPLC purification eluting with 20-75% MeCN+0.1% FA in water+0.1% FA. Desired fractions were collected and lyophilized to give (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (15 mg, 17.4 μmol). m/z (ESI-pos) M+1=862.6; 1H NMR (400 MHz, CHLOROFORM-D) δ 11.31 (s, 1H), 7.58-7.44 (m, 7H), 7.15-7.11 (m, 2H), 6.65 (s, 1H), 6.44 (d, J=3.0 Hz, 1H), 6.28 (d, J=3.0 Hz, 1H), 6.00-5.63 (m, 2H), 4.94-4.66 (m, 2H), 4.51-4.07 (m, 4H), 4.01-3.83 (m, 2H), 3.69-3.32 (m, 3H), 3.17-2.78 (m, 4H), 2.33 (s, 3H), 1.89 (t, J=6.1 Hz, 1H), 1.66 (d, J=6.7 Hz, 3H), 1.53-1.48 (m, 2H), 1.20 (d, J=6.0 Hz, 3H).

### Procedure for Synthesis of Compound 114

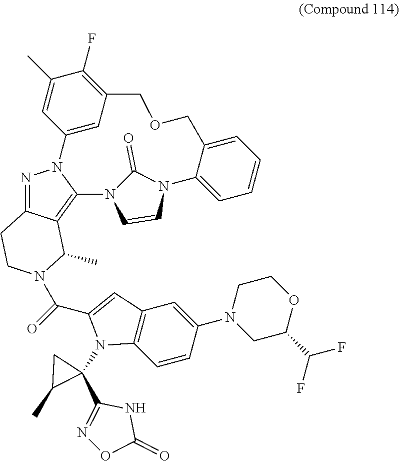

### (8S,10M)-7-({5-[(S)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 114 was synthesized following the procedure of Example 113, (R)-2-(difluoromethyl)morpholine being replaced with(S)-2-(difluoromethyl)morpholine. m/z (ESI-pos) M+1=862.6; 1H NMR (400 MHz, CHLOROFORM-D) δ 11.31 (s, 1H), 7.59-7.42 (m, 7H), 7.15-7.10 (m, 2H), 6.65 (s, 1H), 6.43 (d, J=3.1 Hz, 1H), 6.28 (d, J=3.1 Hz, 1H), 5.95-5.66 (m, 2H), 4.80-4.67 (m, 2H), 4.50-4.21 (m, 2H), 4.18-4.07 (m, 2H), 4.01-3.84 (m, 2H), 3.69-3.31 (m, 3H), 3.16-2.77 (m, 4H), 2.33 (s, 3H), 1.89 (t, J=6.1 Hz, 1H), 1.66 (d, J=6.7 Hz, 3H), 1.53-1.48 (m, 2H), 1.20 (d, J=6.0 Hz, 3H).

### Example 115—Synthesis of Compound 115

### Synthetic Scheme

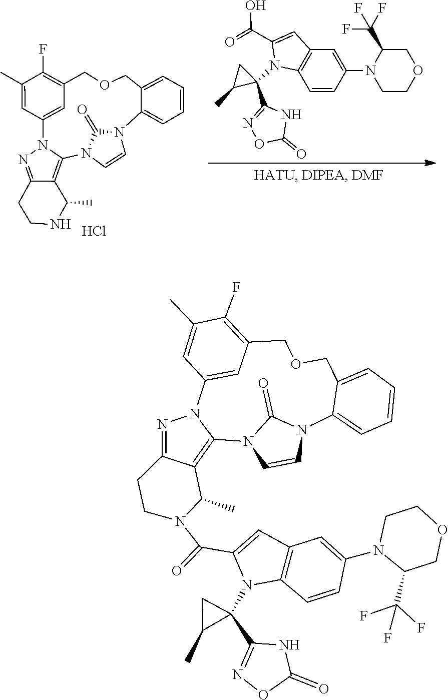

### Procedure for Synthesis of Compound 115

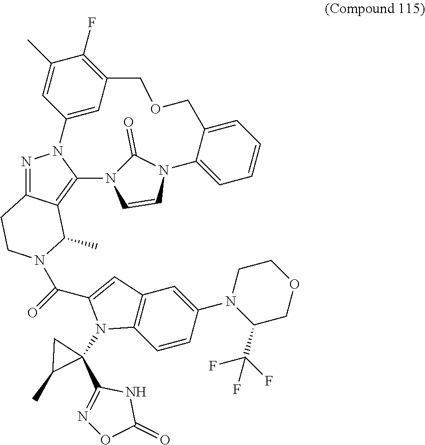

### (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-[(R)-3-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of crude (8S,10M)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (4 mg, 8.3 μmol) and 1-[(1S,2S)-2-methyl-1-(5-oxo-4,5-dihydro-1,2,4-oxadiazol-3-yl)cyclopropyl]-5-[(R)-3-(trifluoromethyl)-4-morpholinyl]-2-indolecarboxylic acid (4 mg, 8.71 μmol) in DMF (0.1 mL) was added N-ethyldiisopropylamine (6 μL, 33.2 μmol) and HATU (5 mg, 12.4 μmol). The mixture stirred at ambient temperature overnight. The reaction mixture was directly loaded onto the reverse phase column and purified by Prep HPLC (Isco ACCQ Prep, Gemini column NX-C18, 250×21 mm, 20 ml/min, 45 minute separation, 20 to 80% H₂O/ACN with 0.1% Formic acid buffer), pure fractions were collected and lyophilized to give (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-[(R)-3-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (4 mg, 4.55 μmol). m/z (ESI-pos) M+1=880.6; 1H NMR (400 MHz, CHLOROFORM-D) δ 11.30 (s, 1H), 7.55-7.44 (m, 6H), 7.17-7.05 (m, 3H), 6.62 (s, 1H), 6.43 (d, J=3.1 Hz, 1H), 6.28 (d, J=3.0 Hz, 1H), 5.79 (q, J=6.7 Hz, 1H), 4.90-4.66 (m, 2H), 4.51-4.24 (m, 3H), 4.17-3.91 (m, 4H), 3.78-3.60 (m, 3H), 3.16-3.00 (m, 3H), 2.33 (s, 3H), 1.89 (t, J=6.0 Hz, 1H), 1.66 (d, J=6.7 Hz, 3H), 1.51-1.48 (m, 2H), 1.19 (d, J=5.9 Hz, 3H)

### Example 116—Synthesis of Compound 116

### Synthetic Scheme

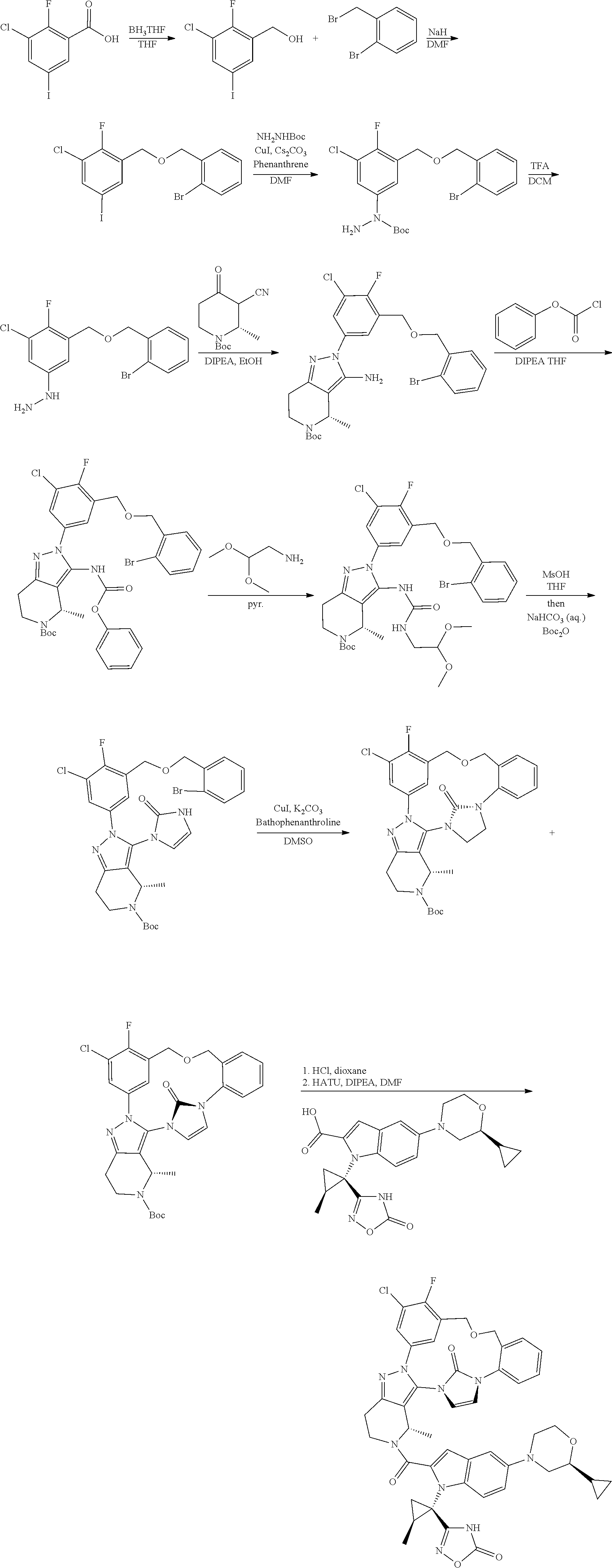

### Procedure for Synthesis of Compound 116

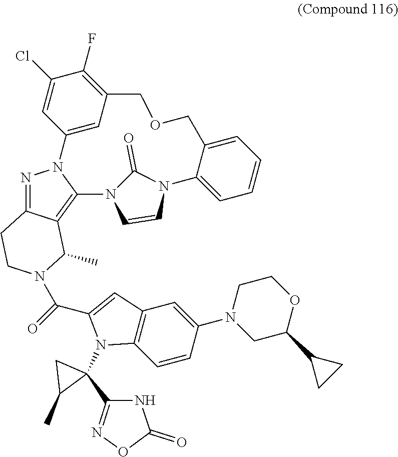

### (8S,10M)-26-chloro-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: 1M Borane in THF (166 mL, 166 mmol) was added dropwise to a stirred solution of 3-chloro-2-fluoro-5-iodobenzoic acid (25 g, 83.2 mmol) in THF (119 mL) cooled to 0° C. The resulting mixture was warmed to room temperature and stirred for an additional 16 h. The mixture was poured into a sat. aq. ammonium chloride solution and extracted with ethyl acetate. The combined organic phases were washed with sat. aq. sodium bicarbonate, brine, dried over Na₂SO₄ and concentrated to afford (3-chloro-2-fluoro-5-iodophenyl)methanol (23 g, 80.3 mmol) which was used in the next step without purification.

Step B: To a solution of (3-chloro-2-fluoro-5-iodophenyl)methanol (20 g, 69.8 mmol) and o-bromo (bromomethyl)benzene (18.3 g, 73.3 mmol) in DMF (233 mL) was added sodium hydride 60% w/w (2.93 g, 73.3 mmol) at 0° C. The reaction was stirred at 0° C. for 30 min and then at ambient temperature for 30 min. The reaction mixture was quenched with sat. aq. NH₄Cl, diluted with ethyl acetate, and washed with brine (2×). The organic layer was dried over Na₂SO₄, filtered and concentrated. The crude product was purified by silica gel column chromatography (0-10% EtOAc/heptane) to give 3-{[(o-bromophenyl)methoxy]methyl}-1-chloro-2-fluoro-5-iodobenzene (22 g, 48.3 mmol) as a clear oil.

Step C: A solution of 3-{[(o-bromophenyl)methoxy]methyl}-1-chloro-2-fluoro-5-iodobenzene (21.7 g, 47.6 mmol), tert-butyl carbazate (8.19 g, 61.9 mmol), dicaesium carbonate (21.7 g, 66.7 mmol), copper iodide (181 mg, 953 μmol), and 4,5-diazaphenanthrene (343 mg, 1.91 mmol) in DMF (60.5 mL) was sparged with Ar for 5 min and heated to 80° C. for 16 h. Upon cooling to rt, the solution was taken up in EtOAc, filtered through Celite, and washed with brine (3×). The organic phase was dried over Na₂SO₄, filtered, and concentrated. The crude product was purified by silica gel chromatography (5-30% EtOAc/heptane) to give tert-butyl 2-(5-{[(o-bromophenyl)methoxy]methyl}-3-chloro-4-fluorophenyl)carbazate (12 g, 26.1 mmol) as a yellow oil. m/z (ESI-pos) M+1=403.1 and 405.1 (loss of tBu).

Step D: To a solution of tert-butyl 2-(5-{[(o-bromophenyl)methoxy]methyl}-3-chloro-4-fluorophenyl)carbazate (1.05 g, 2.28 mmol) in DCM (8.78 mL), trifluoroacetic acid (2.6 mL, 22.8 mmol) was added at ambient temperature. The reaction was stirred for 3 h. The reaction was then quenched with sat. aq. Na₂CO₃ (~20 mL) and extracted with DCM (1×), then washed with water (2×) and brine (1×). The combined aqueous layers were then extracted with 20% IPA/CHCl₃ (2×) and the combined organic layers were dried over Na₂SO₄, filtered, and concentrated to provide (5-{[(o-bromophenyl)methoxy]methyl}-3-chloro-4-fluorophenyl)hydrazine (821 mg, 2.28 mmol) as a yellow oil which was used without purification in the following step. m/z (ESI-pos) M+2=361.1.

Step E: To a solution of crude (5-{[(o-bromophenyl)methoxy]methyl}-3-chloro-4-fluorophenyl)hydrazine (654 mg, 1.36 mmol) in EtOH (5.46 mL) was added N-ethyldiisopropylamine (95 μL, 546 μmol) then tert-butyl(2S)-3-cyano-2-methyl-4-oxo-1-piperidinecarboxylate (390 mg, 1.64 mmol) at ambient temperature. The mixture was then stirred at 80° C. for 20 h. After completion, the reaction mixture was cooled, and the solvent was evaporated under vacuo. The crude material was purified by column chromatography on silica gel (10-60% EtOAc/heptane) to give tert-butyl (S)-3-amino-2-(5-{[(o-bromophenyl)methoxy]methyl}-3-chloro-4-fluorophenyl)-4-methyl-1,2,5-triaza-2,4,6,7-tetrahydro-5H-indene-5-carboxylate (663 mg, 1.14 mmol) as a yellow solid. m/z (ESI-pos) M+2=581.3.

Step F: To a solution of tert-butyl (S)-3-amino-2-(5-{[(o-bromophenyl)methoxy]methyl}-3-chloro-4-fluorophenyl)-4-methyl-1,2,5-triaza-2,4,6,7-tetrahydro-5H-indene-5-carboxylate (580 mg, 1 mmol) in THF (5 mL) was added N-ethyldiisopropylamine (523 μL, 3 mmol) followed by benzenyl chloroformate (251 μL, 2 mmol). The reaction mixture was stirred at room temperature for 30 min. The reaction mixture was directly concentrated under vacuo to give the crude tert-butyl (S)-2-(5-{[(o-bromophenyl)methoxy]methyl}-3-chloro-4-fluorophenyl)-4-methyl-3-[phenyl(oxycarbonylamino)]-1,2,5-triaza-2,4,6,7-tetrahydro-5H-indene-5-carboxylate (0.7 g, 1 mmol) as a yellow oil, which was used directly in the next step without any further purification. m/z (ESI-pos) M+1=701.3 and 703.3.

Step G: Crude tert-butyl tert-butyl (S)-2-(5-{[(o-bromophenyl)methoxy]methyl}-3-chloro-4-fluorophenyl)-4-methyl-3-[phenyl(oxycarbonylamino)]-1,2,5-triaza-2,4,6,7-tetrahydro-5H-indene-5-carboxylate (0.7 g, 1 mmol) was dissolved in pyridine (3.3 mL) and then 2,2-dimethoxyethylamine (436 μL, 4 mmol) was added. The mixture was stirred at room temperature for 30 min and then directly concentrated under vacuo to give the crude residue which was purified by column chromatography (5-80% EtOAc/heptane) to tert-butyl (S)-2-(5-{[(o-bromophenyl)methoxy]methyl}-3-chloro-4-fluorophenyl)-3-[3-(2,2-dimethoxyethyl)ureido]-4-methyl-1,2,5-triaza-2,4,6,7-tetrahydro-5H-indene-5-carboxylate (0.5 g, 703 μmol) as a white solid over 2 steps. m/z (ESI-pos) M+1=712.4 and 713.4.

Step H: To a solution of tert-butyl (S)-2-(5-{[(o-bromophenyl)methoxy]methyl}-3-chloro-4-fluorophenyl)-3-[3-(2,2-dimethoxyethyl)ureido]-4-methyl-1,2,5-triaza-2,4,6,7-tetrahydro-5H-indene-5-carboxylate (0.5 g, 703 μmol) in THF (3.5 mL) was added methanesulfonic acid (137 μL, 2.11 mmol). The mixture was stirred at 55° C. for 1 h and then cooled to ambient temperature. Next, saturated aqueous NaHCO₃ was added until the solution was pH 8 and then tert-butoxycarbonyl-tert-butyl carbonate (169 mg, 774 μmol) was added, and the resulting mixture was stirred at room temperature for 30 min. Upon completion, the reaction mixture was diluted with water and extracted with ethyl acetate (2×). The combined organic layers were washed with brine and dried over anhydrous Na₂SO₄, filtered and concentrated under vacuo. The crude compound was purified by column chromatography (5-90% EtOAc/heptane) to give tert-butyl (8S,10M)-26-chloro-25-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (400 mg, 618 μmol) as an off-white solid. m/z (ESI-pos) M+2=648.3.

Step I: A solution of tert-butyl (S)-2-(5-{[(o-bromophenyl)methoxy]methyl}-3-chloro-4-fluorophenyl)-4-methyl-3-(2-oxo-2,3-dihydro-1H-imidazol-1-yl)-1,2,5-triaza-2,4,6,7-tetrahydro-5H-indene-5-carboxylate (431 mg, 666 μmol), bathophenanthroline (293 mg, 881 μmol), and dipotassium carbonate (304 mg, 2.2 mmol) in DMSO (22.2 mL) was sparged with Ar for 5 minutes and then heated to 130° C. overnight for 21 h. The reaction was diluted with EtOAc and sat. aq. NH₄Cl and then filtered through a pad of celite. The filtrate was partitioned, and the organic phase was washed with brine, dried with sodium sulfate, filtered, concentrated under vacuo. The crude material was purified by silica gel chromatography (5-50% EtOAc/heptane), which provided separation of the atropisomers. The major atropisomer product was tert-butyl (8S,10M)-26-chloro-25-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (112 mg, 198 μmol). m/z (ESI-pos) M+1=566.3 and 567.3.

Step J: 4.0M HCl in dioxane (4 mL) was added to a vial containing tert-butyl (8S,10M)-26-chloro-25-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (112 mg, 198 μmol) and the solution stirred at ambient temperature for 2 h. Upon completion, the solvent was evaporated to give a crude (8S,10M)-26-chloro-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (99 mg, 197 μmol) white solid which was used in the next step without purification. m/z (ESI-pos) M+1=466.2

Step K: To a solution of crude (8S,10M)-26-chloro-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (99 mg, 197 μmol) and 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid (125 mg, 296 μmol) in DMF (2 mL) was added N-ethyldiisopropylamine (137 μL, 788 μmol) and HATU (112 mg, 296 μmol). The mixture stirred at ambient temperature overnight. The reaction mixture was directly loaded onto the reverse phase column and purified by Prep HPLC (Isco ACCQ Prep, Gemini column NX-C18, 250×21 mm, 20 ml/min, 45 minute separation, 20 to 80% H₂O/ACN with 0.1% Formic acid buffer), pure fractions were collected and lyophilized to give (8S,10M)-26-chloro-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (0.1 g, 115 μmol). m/z (ESI-pos) M+1=873.6. 1H NMR (400 MHz, CHLOROFORM-D) δ 11.29 (s, 1H), 7.81-7.70 (m, 1H), 7.57-7.35 (m, 6H), 7.15-7.12 (m, 2H), 6.65 (s, 1H), 6.49 (d, J=3.0 Hz, 1H), 6.33 (d, J=3.0 Hz, 1H), 5.79 (q, J=6.6 Hz, 1H), 4.93-4.65 (m, 2H), 4.55-4.27 (m, 2H), 4.19-4.01 (m, 2H), 3.81-3.32 (m, 4H), 3.16-3.00 (m, 2H), 2.95-2.67 (m, 3H), 1.89 (t, J=5.9 Hz, 1H), 1.64 (d, J=6.7 Hz, 3H), 1.54-1.48 (m, 2H), 1.19 (d, J=5.9 Hz, 3H), 1.00-0.90 (m, 1H), 0.66-0.52 (m, 2H), 0.49-0.42 (m, 1H), 0.33-0.25 (m, 1H).

### Procedure for Synthesis of Compound 117

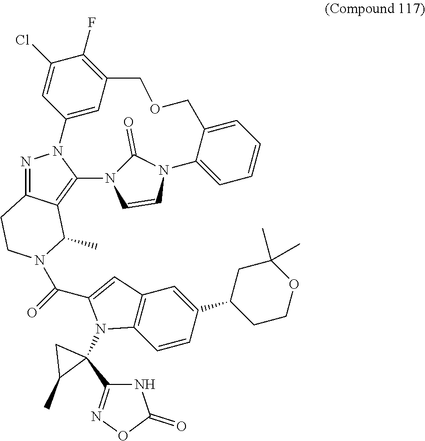

### (8S,10M)-26-chloro-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 117 was synthesized following the procedure of Example 116 with 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid in step K. m/z (ESI-pos) M+1=860.5; 1H NMR (400 MHz, CHLOROFORM-D) δ 11.28 (s, 1H), 7.78-7.74 (m, 1H), 7.61-7.57 (m, 1H), 7.56-7.42 (m, 6H), 7.17-7.13 (m, 1H), 6.69 (s, 1H), 6.49 (d, J=3.1 Hz, 1H), 6.33 (d, J=3.0 Hz, 1H), 5.80 (q, J=6.6 Hz, 1H), 4.83-4.67 (m, 2H), 4.47 (dd, J=13.9, 5.0 Hz, 1H), 4.36 (d, J=9.3 Hz, 1H), 4.18-4.12 (m, 1H), 3.91-3.77 (m, 2H), 3.66-3.57 (m, 1H), 3.19-3.08 (m, 1H), 3.06-2.99 (m, 2H), 1.91 (t, J=5.5 Hz, 1H), 1.79-1.71 (m, 4H), 1.64 (d, J=6.7 Hz, 3H), 1.53-1.49 (m, 2H), 1.33 (s, 3H), 1.27 (s, 3H), 1.20 (d, J=5.7 Hz, 3H).

### Procedure for Synthesis of Compound 118

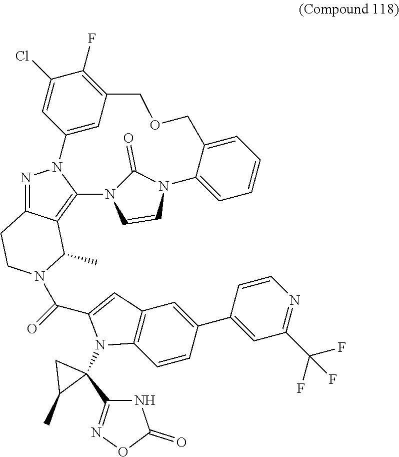

### (8S,10M)-26-chloro-25-fluoro-8-methyl-7-({1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 118 was synthesized following the procedure of Examples 116 and 108 and 105, [5-fluoro-2-(trifluoromethyl)-4-pyridyl]boranediol being replaced with [2-(trifluoromethyl)-4-pyridyl]boranediol. m/z (ESI-pos) M+1=893.5; 1H NMR (400 MHz, CHLOROFORM-D) δ 11.23 (s, 1H), 8.77 (d, J=5.1 Hz, 1H), 8.00-7.91 (m, 2H), 7.79-7.71 (m, 3H), 7.68-7.64 (m, 1H), 7.55-7.44 (m, 4H), 7.18-7.15 (m, 1H), 6.83 (s, 1H), 6.50 (d, J=3.0 Hz, 1H), 6.33 (d, J=3.0 Hz, 1H), 5.83 (q, J=6.7 Hz, 1H), 4.82-4.70 (m, 2H), 4.48 (dd, J=13.8, 5.0 Hz, 1H), 4.36 (d, J=9.3 Hz, 1H), 4.15 (d, J=9.3 Hz, 1H), 3.71-3.63 (m, 1H), 3.20-3.02 (m, 2H), 1.98 (t, J=6.0 Hz, 1H), 1.67 (d, J=6.7 Hz, 3H), 1.63-1.58 (m, 2H), 1.23 (d, J=6.0 Hz, 3H).

### Example 119—Synthesis of Compound 119

### Synthetic Scheme

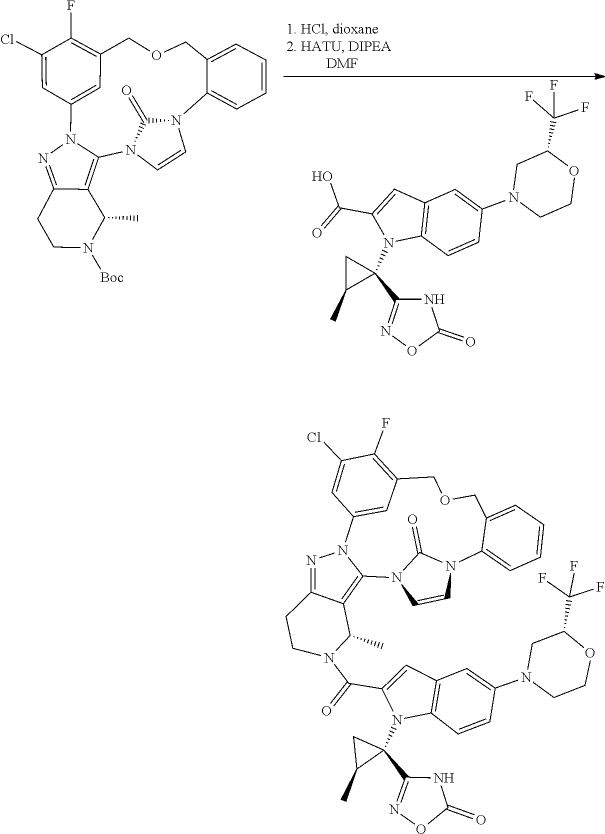

### Procedure for Synthesis of Compound 119

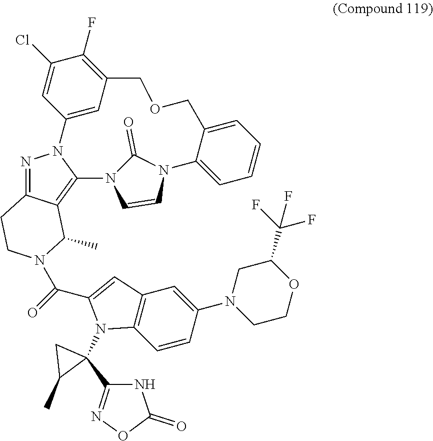

### (8S,10M)-26-chloro-25-fluoro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: 4.0M HCl in dioxane (4 mL) was added to a vial containing tert-butyl (8S,10M)-26-chloro-25-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (112 mg, 198 μmol) and the solution stirred at ambient temperature for 2 h. Upon completion, the solvent was evaporated to give a crude (8S,10M)-26-chloro-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (99 mg, 197 μmol) white solid which was used in the next step without purification. m/z (ESI-pos) M+1=466.2

Step B: To a solution of crude (8S,10M)-26-chloro-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (20 mg, 39.8 μmol) and 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid (19.8 mg, 43.8 μmol) in DMF (0.5 mL) was added N-ethyldiisopropylamine (27.7 μL, 159 μmol) and HATU (22.7 mg, 59.7 μmol). The mixture stirred at ambient temperature overnight. The reaction mixture was directly loaded onto the reverse phase column and purified by Prep HPLC (Isco ACCQ Prep, Gemini column NX-C18, 250×21 mm, 20 ml/min, 45 minute separation, 20 to 80% H2O/ACN with 0.1% Formic acid buffer), pure fractions were collected and lyophilized to give (8S,10M)-26-chloro-25-fluoro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (18 mg, 20 μmol). m/z (ESI-pos) M+1=901.5; ¹H NMR (400 MHz, CHLOROFORM-D) δ 11.27 (s, 1H), 7.79-7.32 (m, 7H), 7.15-7.10 (m, 2H), 6.66 (s, 1H), 6.49 (d, J=3.0 Hz, 1H), 6.32 (d, J=3.0 Hz, 1H), 5.79 (q, J=6.6 Hz, 1H), 4.95-4.25 (m, 4H), 4.17-4.13 (m, 3H), 3.94-3.31 (m, 4H), 3.14-2.84 (m, 4H), 1.90 (t, J=6.0 Hz, 1H), 1.65 (d, J=6.7 Hz, 3H), 1.54-1.48 (m, 2H), 1.20 (d, J=6.0 Hz, 3H).

### Example 120—Synthesis of Compound 120

### Synthetic Scheme

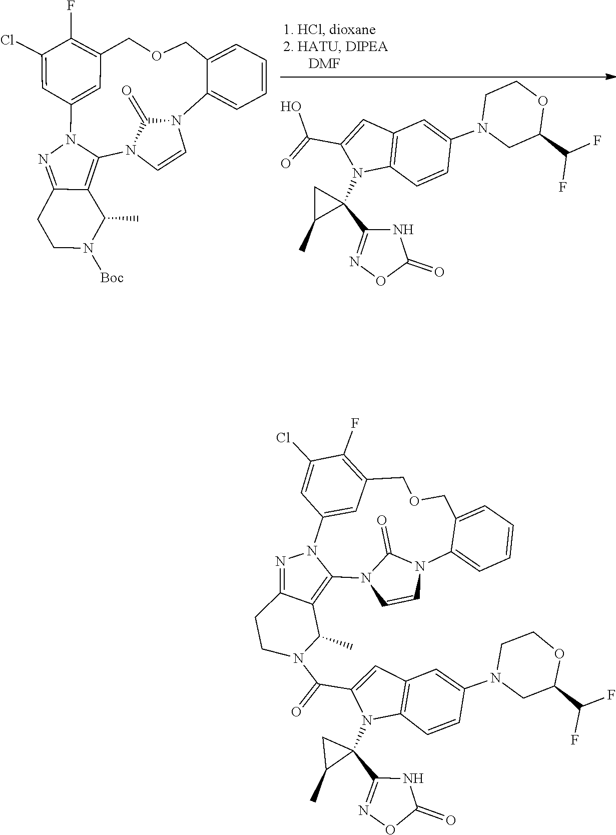

### Procedure for Synthesis of Compound 120

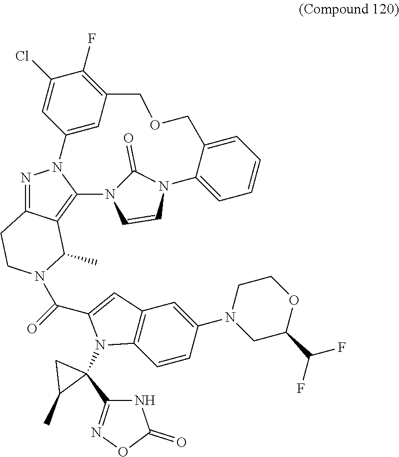

### (8S,10M)-26-chloro-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: 4.0M HCl in dioxane (4 mL) was added to a vial containing tert-butyl (8S,10M)-26-chloro-25-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (112 mg, 198 μmol) and the solution stirred at ambient temperature for 2 h. Upon completion, the solvent was evaporated to give a crude (8S,10M)-26-chloro-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (99 mg, 197 μmol) white solid which was used in the next step without purification. m/z (ESI-pos) M+1=466.2

Step B: To a solution of crude (8S,10M)-26-chloro-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one hydrogen chloride (20 mg, 39.8 μmol) and 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid (19 mg, 43.8 μmol) in DMF (0.5 mL) was added N-ethyldiisopropylamine (27.7 μL, 159 μmol) and HATU (22.7 mg, 59.7 μmol). The mixture stirred at ambient temperature overnight. The reaction mixture was directly loaded onto the reverse phase column and purified by Prep HPLC (Isco ACCQ Prep, Gemini column NX-C18, 250×21 mm, 20 ml/min, 45 minute separation, 20 to 80% H₂O/ACN with 0.1% Formic acid buffer), pure fractions were collected and lyophilized to give (8S,10M)-26-chloro-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (18 mg, 20.4 μmol) m/z (ESI-pos) M+1=883.5; ¹H NMR (400 MHz, CHLOROFORM-D) δ 11.28 (s, 1H), 7.78-7.33 (m, 7H), 7.14-7.11 (m, 2H), 6.66 (s, 1H), 6.49 (d, J=3.0 Hz, 1H), 6.32 (d, J=3.1 Hz, 1H), 5.97-5.64 (m, 2H), 4.91-4.25 (m, 4H), 4.15-3.86 (m, 4H), 3.66-3.32 (m, 3H), 3.14-2.78 (m, 4H), 1.90 (t, J=6.0 Hz, 1H), 1.64 (d, J=6.7 Hz, 3H), 1.53-1.48 (m, 2H), 1.20 (d, J=5.9 Hz, 3H).

### Procedure for Synthesis of Compound 121

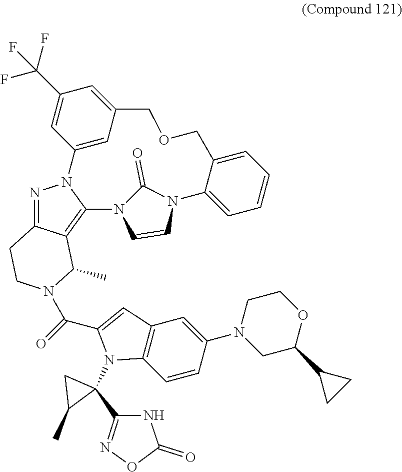

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-8-methyl-26-(trifluoromethyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 121 was synthesized following the procedure of Example 116, (3-chloro-2-fluoro-5-iodophenyl)methanol being replaced with [3-iodo-5-(trifluoromethyl)phenyl]methanol in step B. m/z (ESI-pos) M+1=888.6; ¹H NMR (400 MHz, CHLOROFORM-D) δ 11.29 (s, 1H), 8.03-7.98 (m, 1H), 7.55-7.43 (m, 6H), 7.36-7.32 (m, 1H), 7.17-7.12 (m, 2H), 6.66 (s, 1H), 6.54 (d, J=3.0 Hz, 1H), 6.38 (d, J=3.0 Hz, 1H), 5.81 (q, J=6.6 Hz, 1H), 4.81-4.62 (m, 2H), 4.55-4.28 (m, 2H), 4.12-3.75 (m, 3H), 3.68-3.31 (m, 3H), 3.17-2.70 (m, 5H), 1.89 (t, J=6.0 Hz, 1H), 1.64 (d, J=6.7 Hz, 3H), 1.59-1.55 (m, 2H), 1.20 (d, J=5.9 Hz, 3H), 1.01-0.89 (m, 1H), 0.67-0.51 (m, 2H), 0.50-0.41 (m, 1H), 0.35-0.23 (m, 1H).

### Procedure for Synthesis of Compound 122

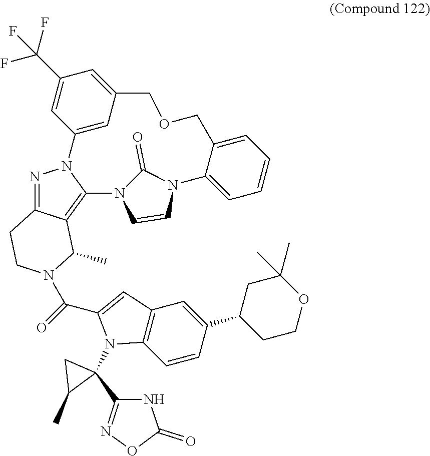

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-26-(trifluoromethyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 122 was synthesized following the procedure of Example 116, (3-chloro-2-fluoro-5-iodophenyl)methanol being replaced with [3-iodo-5-(trifluoromethyl)phenyl]methanol in step B and 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid in step K m/z (ESI-pos) M+1=875.6; 1H NMR (400 MHz, CHLOROFORM-D) δ 11.27 (s, 1H), 8.03-7.94 (m, 1H), 7.61-7.55 (m, 1H), 7.52-7.42 (m, 6H), 7.36-7.27 (m, 2H), 6.70 (s, 1H), 6.54 (d, J=3.1 Hz, 1H), 6.38 (d, J=3.1 Hz, 1H), 5.82 (q, J=6.6 Hz, 1H), 4.81-4.25 (m, 4H), 4.13-3.56 (m, 4H), 3.20-3.02 (m, 3H), 1.91 (t, J=5.5 Hz, 1H), 1.80-1.72 (m, 4H), 1.64 (d, J=6.7 Hz, 3H), 1.61-1.54 (m, 2H), 1.34 (s, 3H), 1.28 (s, 3H), 1.20 (d, J=5.7 Hz, 3H).

### Example 123—Synthesis of Compound 123

### Synthetic Scheme

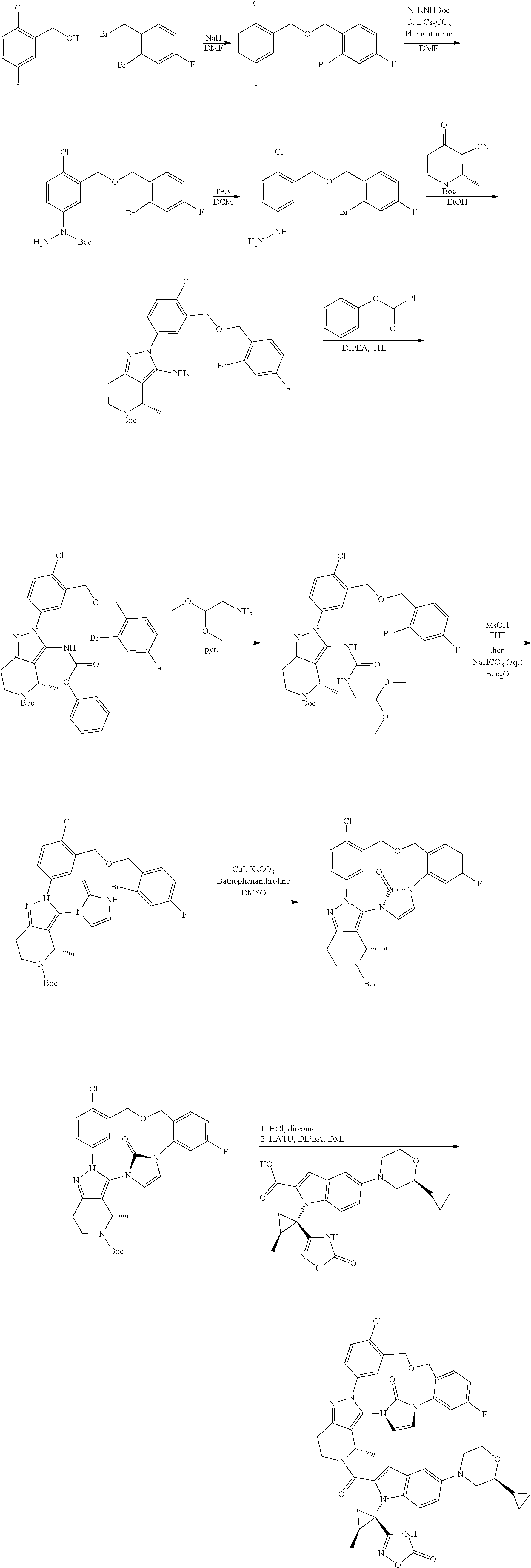

### Procedure for Synthesis of Compound 123

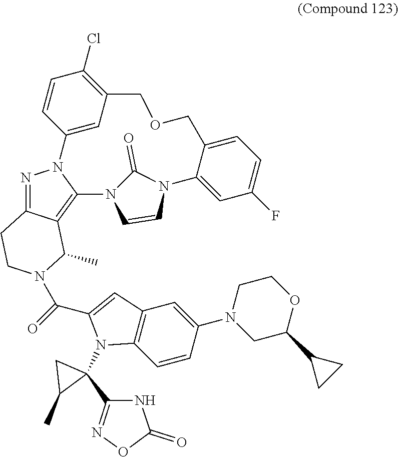

### (8S,10M)-25-chloro-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (2-chloro-5-iodophenyl)methanol (2.36 g, 8.79 mmol) and 2-bromo-1-(bromomethyl)-4-fluorobenzene (2.35 g, 8.79 mmol) in DMF (17.6 mL), sodium hydride was added 60% w/w (369 mg, 9.23 mmol) at 0° C. The reaction was stirred at 0° C. for 30 min and then at ambient temperature for 30 min. The reaction mixture was quenched with sat. aq. NH₄Cl, diluted with ethyl acetate, and washed with brine (2×). The organic layer was dried over Na₂SO₄, filtered and concentrated. The crude product was purified by silica gel column chromatography (0-10% EtOAc/heptane) to give 2-{[(2-bromo-4-fluorophenyl)methoxy]methyl}-1-chloro-4-iodobenzene (3.18 g, 6.98 mmol) as a clear oil.

Step B: A solution of 2-{[(2-bromo-4-fluorophenyl)methoxy]methyl}-1-chloro-4-iodobenzene (3.18 g, 6.98 mmol), tert-butyl carbazate (1.2 g, 9.07 mmol), dicaesium carbonate (3.18 g, 9.76 mmol), copper iodide (133 mg, 697 μmol), and 4,5-diazaphenanthrene (503 mg, 2.79 mmol) in DMF (8.9 mL) was sparged with Ar for 5 min and heated to 80° C. for 15 h. Upon cooling to rt, the solution was taken up in EtOAc, filtered through Celite, and washed with brine (3×). The organic phase was dried over Na₂SO₄, filtered, and concentrated. The crude product was purified by silica gel chromatography (5-30% EtOAc/heptane) to give tert-butyl 2-(3-{[(2-bromo-4-fluorophenyl)methoxy]methyl}-4-chlorophenyl)carbazate (2.65 g, 5.76 mmol) as a yellow oil. m/z (ESI-pos) M+1=383.1 and 405.1 (loss of tBu).

Step C: To a solution of tert-butyl 2-(3-{[(2-bromo-4-fluorophenyl)methoxy]methyl}-4-chlorophenyl)carbazate (2.65 g, 5.76 mmol) in DCM (19.2 mL), trifluoroacetic acid (4.4 mL, 57.6 mmol) was added at ambient temperature. The reaction was stirred for 3 h. The reaction was then quenched with sat. aq. Na₂CO₃ (~20 mL) and extracted with DCM (1×), then washed with water (2×) and brine (1×). The combined aqueous layers were then extracted with 20% IPA/CHCl₃ (2×) and the combined organic layers were dried over Na₂SO₄, filtered, and concentrated to provide (3-{[(2-bromo-4-fluorophenyl)methoxy]methyl}-4-chlorophenyl)hydrazine (1.75 g, 4.87 mmol) as a yellow oil which was used without purification in the following step. m/z (ESI-pos) M+2=361.1.

Step D: To a solution of crude (3-{[(2-bromo-4-fluorophenyl)methoxy]methyl}-4-chlorophenyl)hydrazine (1.75 g, 4.02 mmol) in EtOH (16.1 mL) was added tert-butyl(2S)-3-cyano-2-methyl-4-oxo-1-piperidinecarboxylate (1.15 g, 4.82 mmol) at ambient temperature. The mixture was then stirred at 80° C. for 20 h. After completion, the reaction mixture was cooled, and the solvent was evaporated under vacuo. The crude material was purified by column chromatography on silica gel (10-60% EtOAc/heptane) to give tert-butyl (S)-3-amino-2-(3-{[(2-bromo-4-fluorophenyl)methoxy]methyl}-4-chlorophenyl)-4-methyl-1,2,5-triaza-2,4,6,7-tetrahydro-5H-indene-5-carboxylate (1.93 g, 3.33 mmol) as an off white solid over 2 steps. m/z (ESI-pos) M+2=581.3.

Step E: To a solution of tert-butyl (S)-3-amino-2-(3-{[(2-bromo-4-fluorophenyl)methoxy]methyl}-4-chlorophenyl)-4-methyl-1,2,5-triaza-2,4,6,7-tetrahydro-5H-indene-5-carboxylate (1.92 g, 3.31 mmol) in THF (16.6 mL) was N-ethyldiisopropylamine (1.73 mL, 9.93 mmol) followed by benzenyl chloroformate (831 μL, 6.62 mmol). The reaction mixture was stirred at room temperature for 30 min. The reaction mixture was directly concentrated under vacuo to give the crude tert-butyl (S)-2-(3-{[(2-bromo-4-fluorophenyl)methoxy]methyl}-4-chlorophenyl)-4-methyl-3-[phenyl(oxycarbonylamino)]-1,2,5-triaza-2,4,6,7-tetrahydro-5H-indene-5-carboxylate (2.3 g, 3.29 mmol) as a yellow oil, which was used directly in the next step without any further purification. m/z (ESI-pos) M+1=701.3 and 702.3.

Step F: Crude tert-butyl (S)-2-(3-{[(2-bromo-4-fluorophenyl)methoxy]methyl}-4-chlorophenyl)-4-methyl-3-[phenyl(oxycarbonylamino)]-1,2,5-triaza-2,4,6,7-tetrahydro-5H-indene-5-carboxylate (2.3 g, 3.29 mmol) was dissolved in pyridine (11 mL) and then 2,2-dimethoxyethylamine (1.4 mL, 13.1 mmol) was added. The mixture was stirred at room temperature for 30 min and then directly concentrated under vacuo to give the crude residue which was purified by column chromatography (5-80% EtOAc/heptane) to give tert-butyl (S)-2-(3-{[(2-bromo-4-fluorophenyl)methoxy]methyl}-4-chlorophenyl)-3-[3-(2,2-dimethoxyethyl)ureido]-4-methyl-1,2,5-triaza-2,4,6,7-tetrahydro-5H-indene-5-carboxylate (1.81 g, 2.55 mmol) as a white foam over 2 steps. m/z (ESI-pos) M+1=712.3 and 713.3.

Step G: To a solution of tert-butyl (S)-2-(3-{[(2-bromo-4-fluorophenyl)methoxy]methyl}-4-chlorophenyl)-3-[3-(2,2-dimethoxyethyl)ureido]-4-methyl-1,2,5-triaza-2,4,6,7-tetrahydro-5H-indene-5-carboxylate (1.41 g, 1.98 mmol) in THF (9.9 mL) was added methanesulfonic acid (386 μL, 5.95 mmol). The mixture was stirred at 55° C. for 1 h and then cooled to ambient temperature. Next, saturated aqueous NaHCO₃ was added until the solution was pH 8 and then tert-butoxycarbonyl-tert-butyl carbonate (476 mg, 2.18 mmol) was added, and the resulting mixture was stirred at room temperature for 30 min. Upon completion, the reaction mixture was diluted with water and extracted with ethyl acetate (2×). The combined organic layers were washed with brine and dried over anhydrous Na₂SO₄, filtered and concentrated under vacuo. The crude compound was purified by column chromatography (5-90% EtOAc/heptane) to give tert-butyl (S)-2-(3-{[(2-bromo-4-fluorophenyl)methoxy]methyl}-4-chlorophenyl)-4-methyl-3-(2-oxo-2,3-dihydro-1H-imidazol-1-yl)-1,2,5-triaza-2,4,6,7-tetrahydro-5H-indene-5-carboxylate (1.19 g, 1.84 mmol) as an off-white foam. m/z (ESI-pos) M+2=648.3.

Step H: A solution of tert-butyl (S)-2-(3-{[(2-bromo-4-fluorophenyl)methoxy]methyl}-4-chlorophenyl)-4-methyl-3-(2-oxo-2,3-dihydro-1H-imidazol-1-yl)-1,2,5-triaza-2,4,6,7-tetrahydro-5H-indene-5-carboxylate (0.5 g, 773 μmol), copper iodide (110 mg, 580 μmol), bathophenanthroline (257 mg, 773 μmol), and dipotassium carbonate (267 mg, 1.93 mmol) in DMSO (25.8 mL) was sparged with Ar for 5 minutes and then heated to 130° C. overnight for 40 h. The reaction was diluted with EtOAc and sat. aq. NH₄Cl and then filtered through a pad of celite. The filtrate was partitioned, and the organic phase was washed with brine, dried with sodium sulfate, filtered, concentrated under vacuo. The crude material was purified by silica gel chromatography (5-50% EtOAc/heptane), which provided separation of the atropisomers. The major atropisomer product was tert-butyl (8S,10M)-25-chloro-17-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (132 mg, 233 μmol). m/z (ESI-pos) M+1=566.3 and 567.3.

Step I: 4.0M HCl in dioxane (4 mL) was added to a vial containing tert-butyl (8S,10M)-25-chloro-17-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (129 mg, 228 μmol) and the solution stirred at ambient temperature for 1 h. Upon completion, the solvent was evaporated to give (8S,10M)-25-chloro-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (114 mg, 227 μmol) as a crude white residue which was used in the next step without purification. m/z (ESI-pos) M+1=466.2

Step J: To a solution of crude (8S,10M)-25-chloro-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (25 mg, 49.8 μmol) and 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid (25.3 mg, 59.7 μmol) in DMF (0.5 mL) was added N-ethyldiisopropylamine (34.7 μL, 199 μmol) and HATU (28.4 mg, 74.6 μmol). The mixture stirred at ambient temperature overnight. The reaction mixture was directly loaded onto the reverse phase column and purified by Prep HPLC (Isco ACCQ Prep, Gemini column NX-C18, 250×21 mm, 20 ml/min, 45 minute separation, 20 to 80% H₂O/ACN with 0.1% Formic acid buffer), pure fractions were collected and lyophilized to give (8S,10M)-25-chloro-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (22 mg, 25.2 μmol). m/z (ESI-pos) M+1=872.6 and 873.6. 1H NMR (400 MHz, CHLOROFORM-D) δ 11.30 (s, 1H), 7.65-7.27 (m, 5H), 7.21-7.08 (m, 4H), 6.64 (s, 1H), 6.45 (d, J=3.0 Hz, 1H), 6.30 (d, J=3.1 Hz, 1H), 5.79 (q, J=6.6 Hz, 1H), 4.78 (q, J=15.1 Hz, 2H), 4.53-4.44 (m, 1H), 4.35-4.00 (m, 3H), 3.83-3.29 (m, 4H), 3.18-2.65 (m, 5H), 1.89 (t, J=6.0 Hz, 1H), 1.66 (d, J=6.7 Hz, 3H), 1.53-1.48 (m, 2H), 1.20 (d, J=5.9 Hz, 3H), 1.00-0.91 (m, 1H), 0.66-0.52 (m, 2H), 0.49-0.41 (m, 1H), 0.33-0.25 (m, 1H).

### Procedure for Synthesis of Compound 124

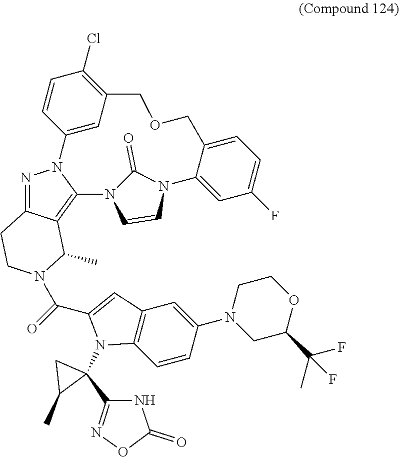

### (8S,10M)-25-chloro-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 124 was synthesized following the procedure of Example 123, 5—[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolecarboxylic acid in step J. m/z (ESI-pos) M+1=896.5, 897.5; 1H NMR (400 MHz, CHLOROFORM-D) δ 11.28 (s, 1H), 7.64-7.59 (m, 1H), 7.58-7.51 (m, 2H), 7.49-7.45 (m, 1H), 7.32-7.28 (m, 1H), 7.22-7.13 (m, 4H), 6.65 (s, 1H), 6.45 (d, J=3.1 Hz, 1H), 6.30 (d, J=3.0 Hz, 1H), 5.79 (q, J=6.7 Hz, 1H), 4.78 (q, J=15.1 Hz, 2H), 4.52-4.25 (m, 2H), 4.20-3.84 (m, 4H), 3.69-3.53 (m, 2H), 3.40-2.99 (m, 3H), 2.92-2.73 (m, 2H), 1.89 (t, J=6.0 Hz, 1H), 1.70-1.64 (m, 6H), 1.52-1.48 (m, 2H), 1.20 (d, J=6.0 Hz, 3H).

### Procedure for Synthesis of Compound 125

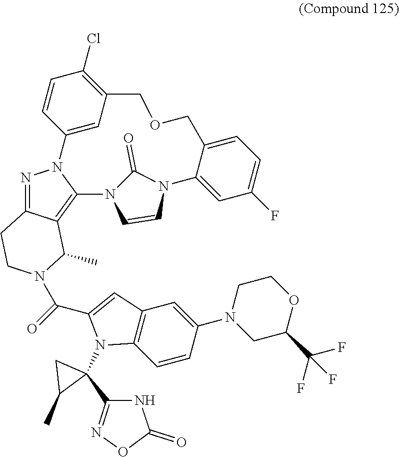

### (8S,10M)-25-chloro-17-fluoro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28), 3,9,12,15,17,19,24,26-nonaen-29-one

Compound 125 was synthesized following the procedure of Example 123, 5—[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid in step J. m/z (ESI-pos) M+1=900.6, 901.6; 1H NMR (400 MHz, CHLOROFORM-D) δ 11.27 (s, 1H), 7.60-7.45 (m, 4H), 7.33-7.28 (m, 1H), 7.21-7.12 (m, 4H), 6.67-6.62 (m, 1H), 6.45 (d, J=3.1 Hz, 1H), 6.30 (d, J=3.1 Hz, 1H), 5.79 (q, J=6.7 Hz, 1H), 4.84-4.73 (m, 2H), 4.50-4.14 (m, 5H), 3.92-3.34 (m, 4H), 3.18-3.00 (m, 2H), 2.95-2.83 (m, 2H), 1.90 (t, J=6.1 Hz, 1H), 1.66 (d, J=6.7 Hz, 3H), 1.53-1.49 (m, 2H), 1.20 (d, J=6.0 Hz, 3H).

### Procedure for Synthesis of Compound 126

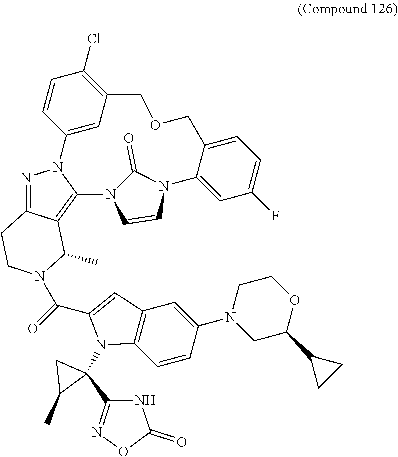

### (8S,10M)-25-chloro-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 126 was synthesized following the procedure of Example 123, 2-bromo-1-(bromomethyl)-4-fluorobenzene being replaced with o-bromo (bromomethyl)benzene in step A. m/z (ESI-pos) M+1=854.6, 855.6; ¹H NMR (400 MHz, CHLOROFORM-D) δ 11.31 (s, 1H), 7.63-7.46 (m, 7H), 7.38-7.30 (m, 1H), 7.17-7.12 (m, 2H), 6.66-6.62 (m, 1H), 6.49-6.38 (m, 1H), 6.31-6.15 (m, 1H), 5.91-5.75 (m, 1H), 4.85-4.74 (m, 2H), 4.52-4.01 (m, 4H), 3.83-3.32 (m, 4H), 3.15-2.98 (m, 2H), 2.94-2.65 (m, 3H), 1.89 (t, J=6.0 Hz, 1H), 1.66 (d, J=6.7 Hz, 3H), 1.56-1.48 (m, 2H), 1.19 (d, J=5.9 Hz, 3H), 0.98-0.93 (m, 1H), 0.64-0.53 (m, 2H), 0.50-0.42 (m, 1H), 0.33-0.25 (m, 1H).

### Procedure for Synthesis of Compound 127

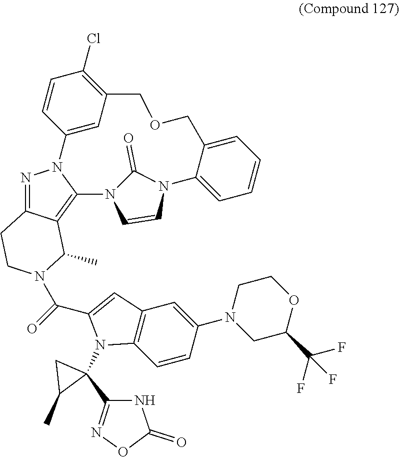

### (8S,10M)-25-chloro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 127 was synthesized following the procedure of Example 123, 2-bromo-1-(bromomethyl)-4-fluorobenzene being replaced with o-bromo (bromomethyl)benzene in step A and 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid in step J. m/z (ESI-pos) M+1=882.5, 883.5; ¹H NMR (400 MHz, CHLOROFORM-D) δ 11.29 (s, 1H), 7.64-7.55 (m, 3H), 7.52-7.46 (m, 4H), 7.32 (d, J=2.7 Hz, 1H), 7.17-7.09 (m, 2H), 6.66 (s, 1H), 6.47 (d, J=3.1 Hz, 1H), 6.29 (d, J=3.0 Hz, 1H), 5.80 (q, J=6.6 Hz, 1H), 4.86-4.73 (m, 2H), 4.53-3.85 (m, 6H), 3.68-3.32 (m, 3H), 3.17-2.83 (m, 4H), 1.90 (t, J=6.2 Hz, 1H), 1.67 (d, J=6.7 Hz, 3H), 1.52-1.48 (m, 2H), 1.20 (d, J=6.0 Hz, 3H).

### Procedure for Synthesis of Compound 128

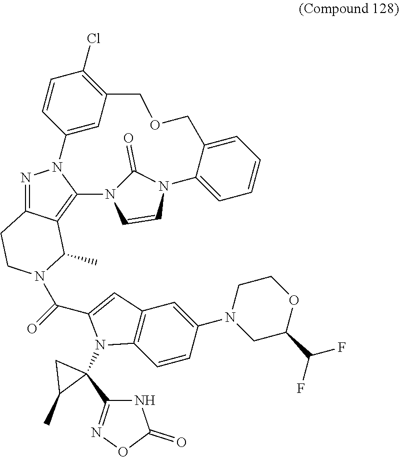

### (8S,10M)-25-chloro-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 128 was synthesized following the procedure of Example 123, 2-bromo-1-(bromomethyl)-4-fluorobenzene being replaced with o-bromo (bromomethyl)benzene in step A and 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid in step J. m/z (ESI-pos) M+1=864.6, 865.6; ¹H NMR (400 MHz, CHLOROFORM-D) δ 11.30 (s, 1H), 7.64-7.55 (m, 3H), 7.52-7.46 (m, 4H), 7.32 (d, J=2.7 Hz, 1H), 7.14-7.10 (m, 2H), 6.65 (s, 1H), 6.47 (d, J=3.0 Hz, 1H), 6.29 (d, J=3.0 Hz, 1H), 5.97-5.65 (m, 2H), 4.80 (q, J=15.2 Hz, 2H), 4.53-4.06 (m, 4H), 4.00-3.83 (m, 2H), 3.69-3.33 (m, 3H), 3.19-2.76 (m, 4H), 1.90 (t, J=6.1 Hz, 1H), 1.66 (d, J=6.7 Hz, 3H), 1.52-1.47 (m, 2H), 1.20 (d, J=6.0 Hz, 3H).

### Procedure for Synthesis of Compound 129

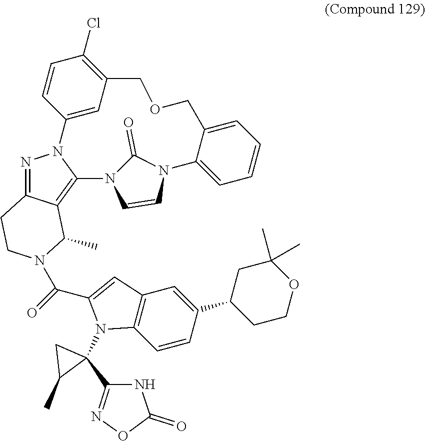

### (8S,10M)-25-chloro-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 129 was synthesized following the procedure of Example 123, 2-bromo-1-(bromomethyl)-4-fluorobenzene being replaced with o-bromo (bromomethyl)benzene in step A and 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid in step J. m/z (ESI-pos) M+1=841.6, 842.6; ¹H NMR (400 MHz, CHLOROFORM-D) δ 11.29 (s, 1H), 7.64-7.46 (m, 9H), 7.39-7.31 (m, 1H), 6.68 (s, 1H), 6.46 (d, J=3.0 Hz, 1H), 6.29 (d, J=3.0 Hz, 1H), 5.80 (q, J=6.7 Hz, 1H), 4.85-4.73 (m, 2H), 4.50-4.17 (m, 3H), 3.90-3.80 (m, 2H), 3.67-3.58 (m, 1H), 3.18-3.01 (m, 3H), 1.91 (t, J=5.7 Hz, 1H), 1.81-1.72 (m, 4H), 1.66 (d, J=5.8 Hz, 3H), 1.65-1.55 (m, 2H), 1.33 (s, 3H), 1.27 (s, 3H), 1.20 (d, J=5.8 Hz, 3H).

### Example 130—Synthesis of Compound 130

### Synthetic Scheme

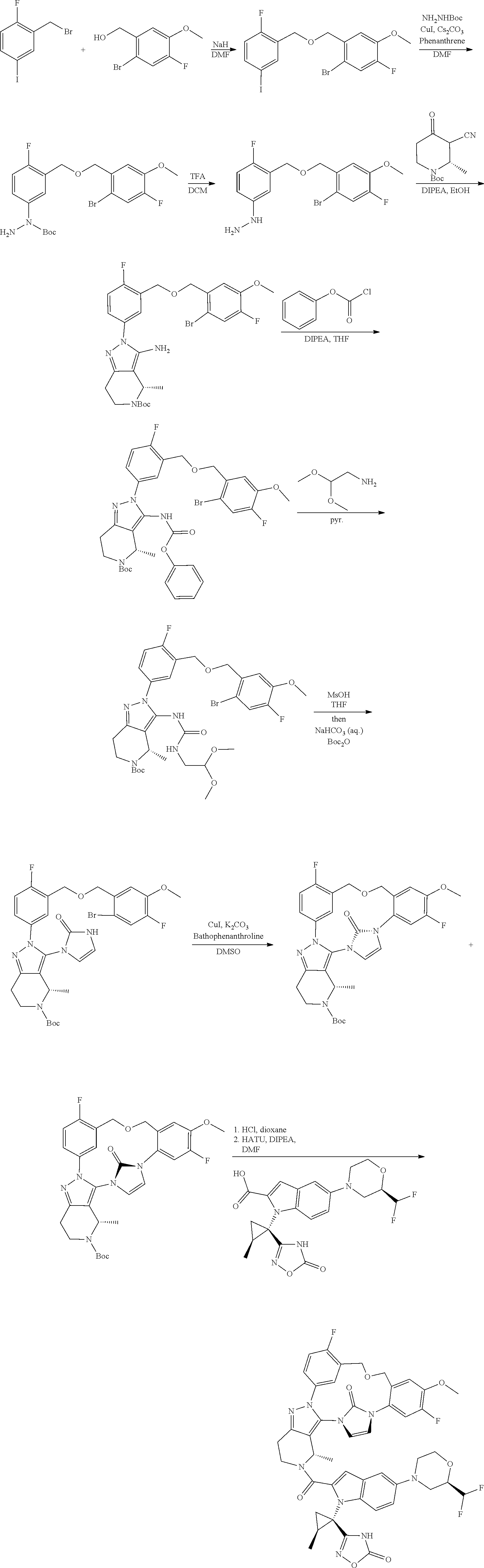

### Procedure for Synthesis of Compound 130

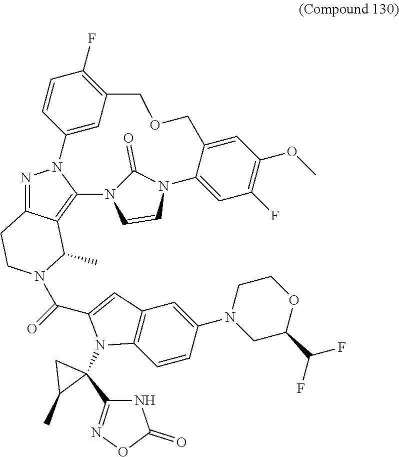

### (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-18-methoxy-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (2-bromo-4-fluoro-5-methoxyphenyl)methanol (1.8 g, 7.27 mmol) and 2-(bromomethyl)-1-fluoro-4-iodobenzene (2.41 g, 7.27 mmol) in DMF (14.5 mL) was added sodium hydride 60% w/w (306 mg, 7.64 mmol) at 0° C. The reaction was stirred at 0° C. for 30 min and then at ambient temperature for 30 min. The reaction mixture was quenched with sat. aq. NH₄Cl, diluted with ethyl acetate, and washed with brine (2×). The organic layer was dried over Na₂SO₄, filtered and concentrated. The crude product was purified by silica gel column chromatography (0-10% EtOAc/heptane) to give 1-bromo-5-fluoro-2-{[(2-fluoro-5-iodophenyl)methoxy]methyl}-4-methoxybenzene (2.77 g, 5.91 mmol) as a white powder.

Step B: A solution of 1-bromo-5-fluoro-2-{[(2-fluoro-5-iodophenyl)methoxy]methyl}-4-methoxybenzene (2.77 g, 5.91 mmol), tert-butyl carbazate (1.01 g, 7.68 mmol), dicaesium carbonate (2.69 g, 8.27 mmol), copper iodide (112 mg, 591 μmol), and 4,5-diazaphenanthrene (426 mg, 2.36 mmol) in DMF (7.5 mL) was sparged with Ar for 5 min and heated to 80° C. for 16 h. Upon cooling to rt, the solution was taken up in EtOAc, filtered through Celite, and washed with brine (3×). The organic phase was dried over Na2SO4, filtered, and concentrated. The crude product was purified by silica gel chromatography (5-30% EtOAc/heptane) to give tert-butyl 2-(3-{[(2-bromo-4-fluoro-5-methoxyphenyl)methoxy]methyl}-4-fluorophenyl)carbazate (2.5 g, 5.28 mmol) as yellow oil. m/z (ESI-pos) M+1=417.1 and 420.1 (loss of tBu).

Step C: To a solution of tert-butyl 2-(3-{[(2-bromo-4-fluoro-5-methoxyphenyl)methoxy]methyl}-4-fluorophenyl)carbazate (2.4 g, 5.07 mmol) in DCM (17 mL), trifluoroacetic acid (3.9 mL, 50.7 mmol) was added at ambient temperature. The reaction was stirred for 3 h. The reaction was then quenched with sat. aq. Na₂CO₃ (~20 mL) and extracted with DCM (1×), then washed with water (2×) and brine (1×). The combined aqueous layers were then extracted with 20% IPA/CHCl₃ (2×) and the combined organic layers were dried over Na₂SO₄, filtered, and concentrated to provide (3-{[(2-bromo-4-fluoro-5-methoxyphenyl)methoxy]methyl}-4-fluorophenyl)hydrazine (1.6 g, 4.29 mmol) as a yellow oil which was used without purification in the following step. m/z (ESI-pos) M+2=375.1.

Step D: To a solution of crude (3-{[(2-bromo-4-fluoro-5-methoxyphenyl)methoxy]methyl}-4-fluorophenyl)hydrazine (1.6 g, 3.43 mmol) in EtOH (14 mL) was added N-ethyldiisopropylamine (299 μL, 1.71 mmol) then tert-butyl(2S)-3-cyano-2-methyl-4-oxo-1-piperidinecarboxylate (981 mg, 4.12 mmol) at ambient temperature. The mixture was then stirred at 80° C. for 20 h. After completion, the reaction mixture was cooled, and the solvent was evaporated under vacuo. The crude material was purified by column chromatography on silica gel (10-60% EtOAc/heptane) to give tert-butyl (S)-3-amino-2-(3-{[(2-bromo-4-fluoro-5-methoxyphenyl)methoxy]methyl}-4-fluorophenyl)-4-methyl-1,2,5-triaza-2,4,6,7-tetrahydro-5H-indene-5-carboxylate (1.6 g, 2.7 mmol) as an off white solid. m/z (ESI-pos) M+2=595.3.

Step E: To a solution of tert-butyl (S)-3-amino-2-(3-{[(2-bromo-4-fluoro-5-methoxyphenyl)methoxy]methyl}-4-fluorophenyl)-4-methyl-1,2,5-triaza-2,4,6,7-tetrahydro-5H-indene-5-carboxylate (1.5 g, 2.53 mmol) in THF (12.6 mL) was added N-ethyldiisopropylamine (1.32 mL, 7.58 mmol) followed by benzenyl chloroformate (634 μL, 5.1 mmol). The reaction mixture was stirred at room temperature for 30 min. The reaction mixture was directly concentrated under vacuo to give the crude tert-butyl tert-butyl (S)-2-(3-{[(2-bromo-4-fluoro-5-methoxyphenyl)methoxy]methyl}-4-fluorophenyl)-4-methyl-3-[phenyl(oxycarbonylamino)]-1,2,5-triaza-2,4,6,7-tetrahydro-5H-indene-5-carboxylate (1.8 g, 2.52 mmol) as a yellow oil, which was used directly in the next step without any further purification. m/z (ESI-pos) M+2=715.4.

Step F: Crude tert-butyl (S)-2-(3-{[(2-bromo-4-fluoro-5-methoxyphenyl)methoxy]methyl}-4-fluorophenyl)-4-methyl-3-[phenyl(oxycarbonylamino)]-1,2,5-triaza-2,4,6,7-tetrahydro-5H-indene-5-carboxylate (1.8 g, 2.52 mmol) was dissolved in pyridine (8.4 mL) and then 2,2-dimethoxyethylamine (1.1 mL, 10.1 mmol) was added. The mixture was stirred at room temperature for 30 min and then directly concentrated under vacuo to give the crude residue which was purified by column chromatography (5-80% EtOAc/heptane) to afford tert-butyl (S)-2-(3-{[(2-bromo-4-fluoro-5-methoxyphenyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (1.34 g, 1.85 mmol) as a white foam over 2 steps. m/z (ESI-pos) M+1=724.4 and 726.4.

Step G: To a solution of tert-butyl (S)-2-(3-{[(2-bromo-4-fluoro-5-methoxyphenyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (1.8 g, 2.48 mmol) in THF (12.4 mL) was added methanesulfonic acid (484 μL, 7.45 mmol). The mixture was stirred at 55° C. for 1 h and then cooled to ambient temperature. Next, saturated aqueous NaHCO₃ was added until the solution was pH 8 and then tert-butoxycarbonyl-tert-butyl carbonate (596 mg, 2.73 mmol) was added, and the resulting mixture was stirred at room temperature for 30 min. Upon completion, the reaction mixture was diluted with water and extracted with ethyl acetate (2×). The combined organic layers were washed with brine and dried over anhydrous Na2SO4, filtered and concentrated under vacuo. The crude compound was purified by column chromatography (5-90% EtOAc/heptane) to give tert-butyl (S)-2-(3-{[(2-bromo-4-fluoro-5-methoxyphenyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (1.2 g, 1.82 mmol) as an off-white solid. m/z (ESI-pos) M+2=662.3.

Step H: A solution of tert-butyl (S)-2-(3-{[(2-bromo-4-fluoro-5-methoxyphenyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (0.6 g, 908 μmol), bathophenanthroline (302 mg, 908 μmol), and dipotassium carbonate (314 mg, 2.3 mmol) in DMSO (30 mL) was sparged with Ar for 5 minutes and then heated to 130° C. overnight for 40 h. The reaction was diluted with EtOAc and sat. aq. NH₄Cl and then filtered through a pad of celite. The filtrate was partitioned, and the organic phase was washed with brine, dried with sodium sulfate, filtered, concentrated under vacuo. The crude material was purified by silica gel chromatography (5-50% EtOAc/heptane), which provided separation of the atropisomers. The major atropisomer product was tert-butyl (8S,10M)-17,25-difluoro-18-methoxy-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (140 mg, 242 μmol) m/z (ESI-pos) M+1=580.4.

Step I: 4.0M HCl in dioxane (4 mL) was added to a vial containing tert-butyl (8S,10M)-17,25-difluoro-18-methoxy-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (0.1 g, 173 μmol) and the solution stirred at ambient temperature for 2 h. Upon completion, the solvent was evaporated to give a crude (8S,10M)-17,25-difluoro-18-methoxy-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (89 mg, 172 μmol) white solid which was used in the next step without purification. m/z (ESI-pos) M+1=480.3

Step J: To a solution (8S,10M)-17,25-difluoro-18-methoxy-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (20 mg, 38.8 μmol) and 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid (18.5 mg, 42.6 μmol) in DMF (0.5 mL) was added N-ethyldiisopropylamine (27 μL, 155 μmol) and HATU (22.1 mg, 58.1 μmol). The mixture stirred at ambient temperature overnight. The reaction mixture was directly loaded onto the reverse phase column and purified by Prep HPLC (Isco ACCQ Prep, Gemini column NX-C18, 250×21 mm, 20 ml/min, 45 minute separation, 20 to 80% H2O/ACN with 0.1% Formic acid buffer), pure fractions were collected and lyophilized to give (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-18-methoxy-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (14 mg, 15.6 μmol) m/z (ESI-pos) M+1=896.6. 1H NMR (400 MHz, CHLOROFORM-D) δ 11.29 (s, 1H), 7.62-7.31 (m, 3H), 7.20-7.11 (m, 5H), 6.65 (s, 1H), 6.36 (d, J=3.1 Hz, 1H), 6.23 (d, J=3.1 Hz, 1H), 5.97-5.65 (m, 2H), 4.92-4.43 (m, 3H), 4.22-4.08 (m, 3H), 3.96 (s, 3H), 3.94-3.84 (m, 2H), 3.68-3.32 (m, 3H), 3.17-2.78 (m, 4H), 1.89 (t, J=6.1 Hz, 1H), 1.66 (d, J=6.7 Hz, 3H), 1.54-1.48 (m, 2H), 1.20 (d, J=6.0 Hz, 3H).

### Procedure for Synthesis of Compound 131

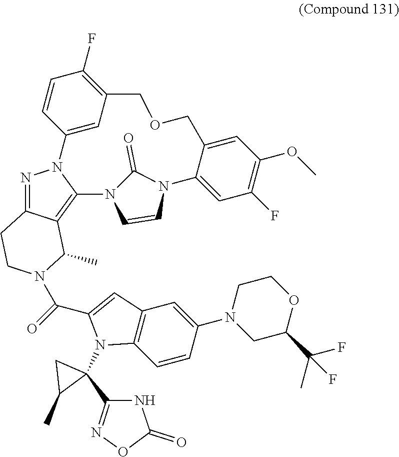

### (8S,10M)-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-18-methoxy-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 131 was synthesized following the procedure of Example 130, 5—[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolecarboxylic acid in step J. m/z (ESI-pos) M+1=910.6; ¹H NMR (400 MHz, CHLOROFORM-D) δ 11.30 (s, 1H), 7.60-7.55 (m, 2H), 7.35-7.32 (m, 1H), 7.19-7.11 (m, 5H), 6.65 (s, 1H), 6.36 (d, J=3.0 Hz, 1H), 6.23 (d, J=3.1 Hz, 1H), 5.78 (q, J=6.7 Hz, 1H), 4.80-4.69 (m, 2H), 4.50-4.07 (m, 4H), 3.96 (s, 3H), 3.91-3.33 (m, 5H), 3.18-2.72 (m, 4H), 1.89 (t, J=6.0 Hz, 1H), 1.68-1.63 (m, 6H), 1.51-1.48 (m, 2H), 1.20 (d, J=5.9 Hz, 3H).

### Procedure for Synthesis of Compound 132

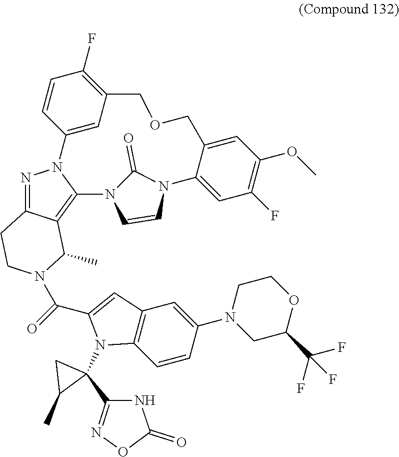

### (8S,10M)-17,25-difluoro-18-methoxy-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 132 was synthesized following the procedure of Example 130, 5—[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid in step J. m/z (ESI-pos) M+1=914.6; ¹H NMR (400 MHz, CHLOROFORM-D) δ 11.28 (s, 1H), 7.63-7.30 (m, 3H), 7.20-7.11 (m, 5H), 6.65 (s, 1H), 6.36 (d, J=3.1 Hz, 1H), 6.23 (d, J=3.0 Hz, 1H), 5.79 (q, J=6.6 Hz, 1H), 4.92-4.44 (m, 3H), 4.20-4.10 (m, 4H), 3.96 (s, 3H), 3.94-3.85 (m, 1H), 3.68-3.33 (m, 3H), 3.16-2.84 (m, 4H), 1.90 (t, J=6.1 Hz, 1H), 1.66 (d, J=6.7 Hz, 3H), 1.54-1.48 (m, 2H), 1.20 (d, J=6.0 Hz, 3H).

### Procedure for Synthesis of Compound 133

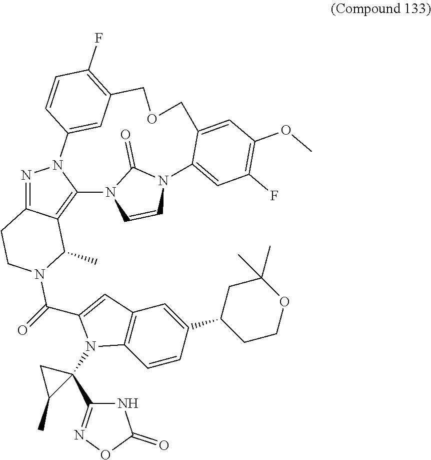

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,25-difluoro-18-methoxy-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 133 was synthesized following the procedure of Example 130, 5—[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid in step J. m/z (ESI-pos) M+1=873.6; ¹H NMR (400 MHz, CHLOROFORM-D) δ 11.29 (s, 1H), 7.62-7.31 (m, 5H), 7.22-7.11 (m, 3H), 6.71-6.67 (m, 1H), 6.36 (d, J=3.0 Hz, 1H), 6.23 (d, J=3.1 Hz, 1H), 5.79 (q, J=6.6 Hz, 1H), 4.94-4.41 (m, 3H), 4.23-4.06 (m, 2H), 3.96 (s, 3H), 3.90-3.55 (m, 3H), 3.18-2.99 (m, 3H), 1.91 (t, J=5.7 Hz, 1H), 1.80-1.71 (m, 4H), 1.66 (d, J=6.6 Hz, 3H), 1.53-1.48 (m, 2H), 1.33 (s, 3H), 1.27 (s, 3H), 1.20 (d, J=5.8 Hz, 3H).

### Procedure for Synthesis of Compound 134

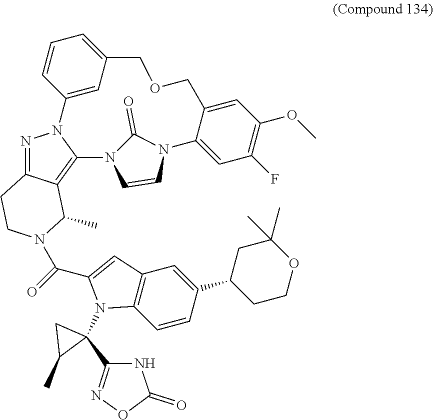

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17-fluoro-18-methoxy-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 134 was synthesized following the procedure of Example 130, 2-(bromomethyl)-1-fluoro-4-iodobenzene being replaced with m-(bromomethyl) iodobenzene in step A and 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid in step J. m/z (ESI-pos) M+1=855.6; ¹H NMR (400 MHz, CHLOROFORM-D) δ 11.31 (s, 1H), 7.63-7.57 (m, 2H), 7.51-7.44 (m, 2H), 7.29-7.26 (m, 2H), 7.21-7.09 (m, 3H), 6.69 (s, 1H), 6.36 (d, J=3.0 Hz, 1H), 6.24 (d, J=3.0 Hz, 1H), 5.85-5.77 (m, 1H), 4.76-4.42 (m, 3H), 4.21-4.02 (m, 2H), 3.95 (s, 3H), 3.89-3.58 (m, 3H), 3.20-3.00 (m, 3H), 1.91 (t, J=5.7 Hz, 1H), 1.81-1.71 (m, 4H), 1.66 (d, J=6.5 Hz, 3H), 1.64-1.58 (m, 2H), 1.33 (s, 3H), 1.27 (s, 3H), 1.20 (d, J=5.8 Hz, 3H).

### Procedure for Synthesis of Compound 135

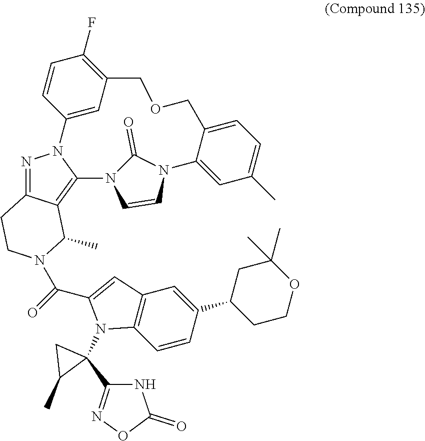

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-8,17-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 135 was synthesized following the procedure of Example 130, (2-bromo-4-fluoro-5-methoxyphenyl)methanol being replaced with (2-bromo-4-tolyl)methanol in step A and 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid in step J. m/z (ESI-pos) M+1=839.6; ¹H NMR (400 MHz, CHLOROFORM-D) δ 11.32 (s, 1H), 7.63-7.49 (m, 4H), 7.45-7.39 (m, 1H), 7.34-7.30 (m, 1H), 7.28-7.26 (m, 2H), 7.19-7.14 (m, 1H), 6.69 (s, 1H), 6.40 (d, J=3.1 Hz, 1H), 6.24 (d, J=3.0 Hz, 1H), 5.79 (q, J=6.6 Hz, 1H), 4.78-4.64 (m, 2H), 4.46 (dd, J=13.8, 5.1 Hz, 1H), 4.25 (d, J=9.2 Hz, 1H), 4.12 (d, J=9.3 Hz, 1H), 3.89-3.80 (m, 2H), 3.67-3.59 (m, 1H), 3.19-3.09 (m, 1H), 3.05-3.00 (m, 2H), 2.42 (s, 3H), 1.90 (t, J=5.7 Hz, 1H), 1.81-1.72 (m, 4H), 1.67 (d, J=6.7 Hz, 3H), 1.55-1.52 (m, 2H), 1.33 (s, 3H), 1.27 (s, 3H), 1.20 (d, J=5.8 Hz, 3H).

### Example 136—Synthesis of Compound 136

### Synthetic Scheme

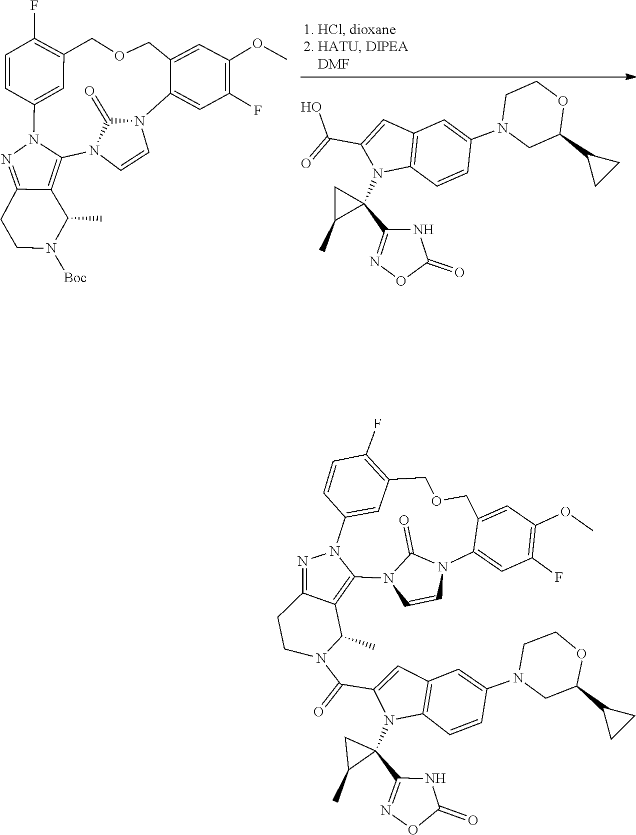

### Procedure for Synthesis of Compound 136

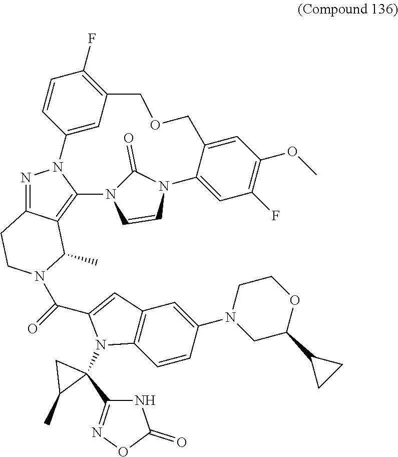

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-18-methoxy-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: 4.0M HC in dioxane (4 mL) was added to a vial containing tert-butyl (8S,10M)-17,25-difluoro-18-methoxy-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (0.1 g, 173 μmol) and the solution stirred at ambient temperature for 2 h. Upon completion, the solvent was evaporated to give a crude (8S,10M)-17,25-difluoro-18-methoxy-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (89 mg, 172 μmol) white solid which was used in the next step without purification. m/z (ESI-pos) M+1=480.3

Step B: To a solution (8S,10M)-17,25-difluoro-18-methoxy-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (25 mg, 48.5 μmol) and 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid (22.6 mg, 53.3 μmol) in DMF (0.5 mL) was added N-ethyldiisopropylamine (34 μL, 194 μmol) and HATU (27.6 mg, 72.7 μmol). The mixture stirred at ambient temperature overnight. The reaction mixture was directly loaded onto the reverse phase column and purified by Prep HPLC (Isco ACCQ Prep, Gemini column NX-C18, 250×21 mm, 20 ml/min, 45 minute separation, 20 to 80% H₂O/ACN with 0.1% Formic acid buffer), pure fractions were collected and lyophilized to give (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-18-methoxy-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (18 mg, 20.3 μmol) m/z (ESI-pos) M+1=886.6; ¹H NMR (400 MHz, CHLOROFORM-D) δ 11.30 (s, 1H), 7.66-7.54 (m, 2H), 7.36-7.32 (m, 1H), 7.20-7.12 (m, 5H), 6.64 (s, 1H), 6.36 (d, J=3.1 Hz, 1H), 6.23 (d, J=3.0 Hz, 1H), 5.78 (q, J=6.6 Hz, 1H), 4.80-4.69 (m, 2H), 4.55-4.02 (m, 4H), 3.96 (s, 3H), 3.82-3.58 (m, 2H), 3.54-3.30 (m, 2H), 3.17-2.99 (m, 2H), 2.96-2.67 (m, 3H), 1.89 (t, J=6.0 Hz, 1H), 1.66 (d, J=6.7 Hz, 3H), 1.51-1.44 (m, 2H), 1.20 (d, J=5.9 Hz, 3H), 1.00-0.91 (m, 1H), 0.65-0.51 (m, 2H), 0.50-0.41 (m, 1H), 0.34-0.24 (m, 1H).

### Procedure for Synthesis of Compound 137

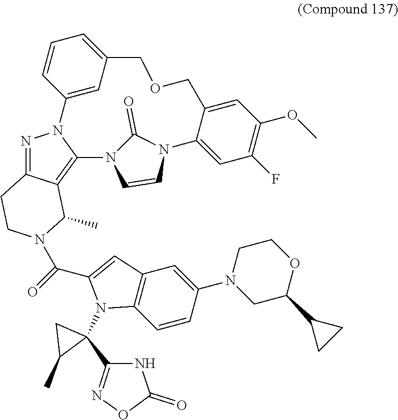

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-18-methoxy-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 137 was synthesized following the procedure of Example 130, 2-(bromomethyl)-1-fluoro-4-iodobenzene being replaced with m-(bromomethyl) iodobenzene in step A and 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid. m/z (ESI-pos) M+1=868.6; ¹H NMR (400 MHz, CHLOROFORM-D) δ 11.32 (s, 1H), 7.63-7.28 (m, 4H), 7.22-7.12 (m, 5H), 6.65 (s, 1H), 6.36 (d, J=3.0 Hz, 1H), 6.25 (d, J=3.1 Hz, 1H), 5.79 (q, J=6.6 Hz, 1H), 4.75-4.44 (m, 3H), 4.20-4.02 (m, 3H), 3.95 (s, 3H), 3.82-3.48 (m, 3H), 3.38-3.00 (m, 3H), 2.95-2.67 (m, 3H), 1.89 (t, J=6.1 Hz, 1H), 1.66 (d, J=6.7 Hz, 3H), 1.52-1.48 (m, 2H), 1.20 (d, J=6.0 Hz, 3H), 1.00-0.91 (m, 1H), 0.64-0.51 (m, 2H), 0.49-0.42 (m, 1H), 0.33-0.25 (m, 1H).

### Procedure for Synthesis of Compound 138

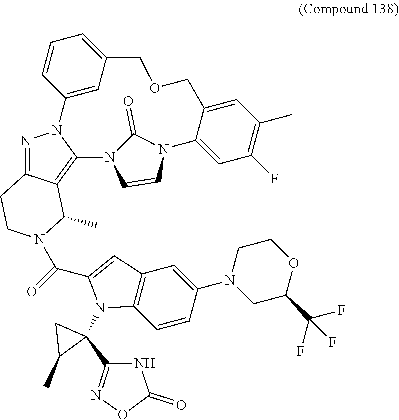

### (8S,10M)-17-fluoro-8,18-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 138 was synthesized following the procedure of Example 130, 2-(bromomethyl)-1-fluoro-4-iodobenzene being replaced with m-(bromomethyl) iodobenzene, (2-bromo-4-fluoro-5-methoxyphenyl)methanol being replaced with (2-bromo-5-fluoro-4-tolyl)methanol step A and 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid in step J. m/z (ESI-pos) M+1=880.6; ¹H NMR (400 MHz, CHLOROFORM-D) δ 11.31 (s, 1H), 7.62-7.34 (m, 4H), 7.21-7.10 (m, 5H), 6.66 (s, 1H), 6.38 (d, J=3.1 Hz, 1H), 6.26 (d, J=3.0 Hz, 1H), 5.80 (q, J=6.6 Hz, 1H), 4.73-4.42 (m, 3H), 4.20-4.14 (m, 2H), 4.05-3.86 (m, 2H), 3.67-3.50 (m, 2H), 3.43-2.82 (m, 6H), 2.32 (s, 3H), 1.90 (t, J=6.2 Hz, 1H), 1.66 (d, J=6.7 Hz, 3H), 1.54-1.49 (m, 2H), 1.20 (d, J=6.0 Hz, 3H).

### Procedure for Synthesis of Compound 139

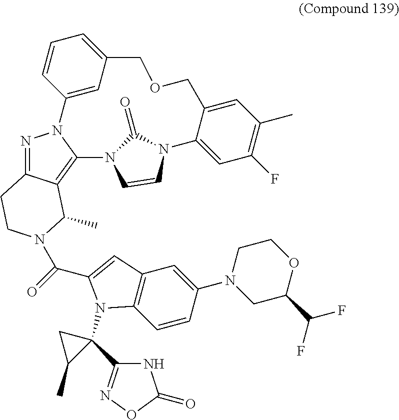

### (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 139 was synthesized following the procedure of Example 130, 2-(bromomethyl)-1-fluoro-4-iodobenzene being replaced with m-(bromomethyl) iodobenzene and (2-bromo-4-fluoro-5-methoxyphenyl)methanol being replaced with (2-bromo-5-fluoro-4-tolyl)methanol step A. m/z (ESI-pos) M+1=862.6; ¹H NMR (400 MHz, CHLOROFORM-D) δ 11.32 (s, 1H), 7.65-7.54 (m, 2H), 7.49-7.32 (m, 2H), 7.21-7.10 (m, 5H), 6.66 (s, 1H), 6.38 (d, J=3.0 Hz, 1H), 6.26 (d, J=3.0 Hz, 1H), 5.97-5.64 (m, 2H), 4.72-4.42 (m, 3H), 4.21-4.02 (m, 3H), 3.98-3.86 (m, 2H), 3.67-3.33 (m, 3H), 3.18-3.01 (m, 2H), 2.94-2.77 (m, 2H), 2.31 (s, 3H), 1.89 (t, J=6.1 Hz, 1H), 1.67 (d, J=6.7 Hz, 3H), 1.56-1.49 (m, 2H), 1.20 (d, J=6.0 Hz, 3H).

### Example 140—Synthesis of Compound 140

### Synthetic Scheme

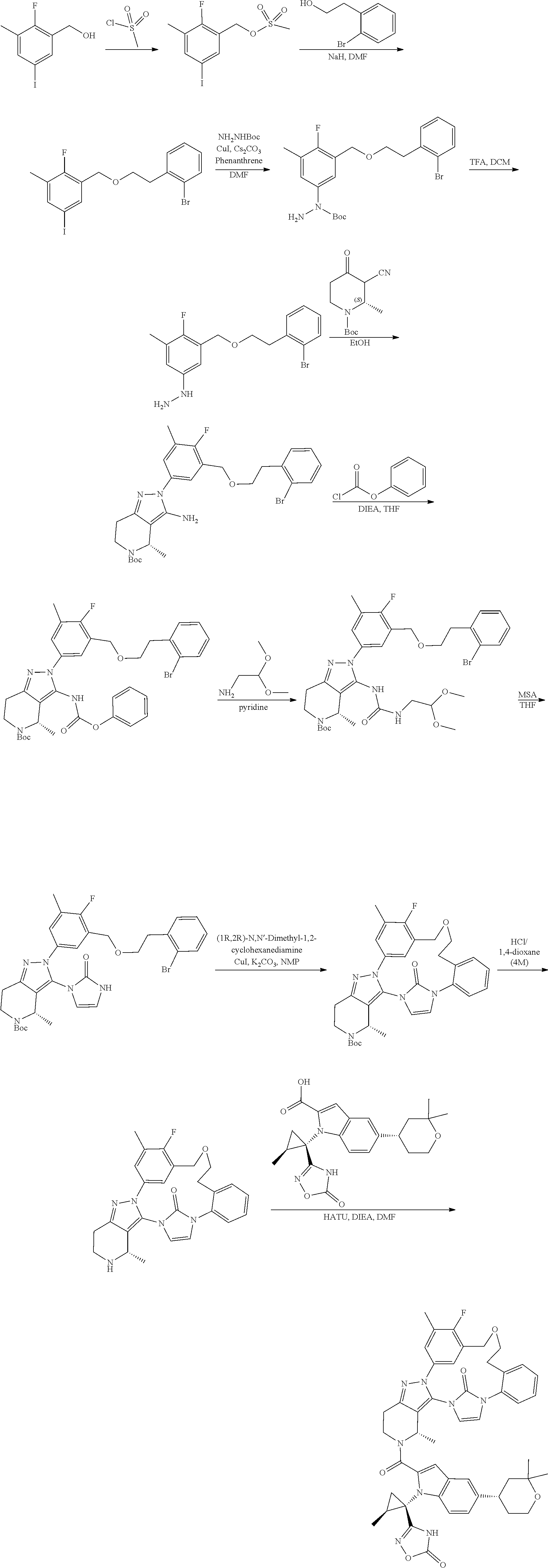

### Procedure for Synthesis of Compound 140

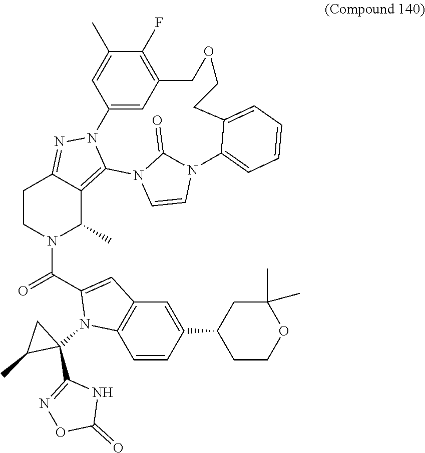

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8,27-dimethyl-23-oxa-2,3,7,11,14-pentaazahexacyclo[22.3.1.1¹¹,¹⁴.0²,¹⁰.0⁴,⁹.0¹⁵,²⁰]triaconta-1(29),3,9,12,15,17,19,25,27-nonaen-30-one

Step A: To a solution of (2-fluoro-5-iodo-3-tolyl)methanol (1 g, 3.76 mmol), triethylamine (786 μL, 5.64 mmol) in dichloromethane (19 mL) at 0° C. was added (chlorosulfonyl)methane (320 μL, 4.13 mmol). The mixture was stirred at 0° C. for 30 minutes, LCMS showed full conversion of desired product. The reaction mixture was diluted with H2O and extracted with dichloromethane (2×). The organics were washed with brine, dried with sodium sulfate, filtered and concentrated in vacuo to afford 2-fluoro-5-iodo-3-[(mesyloxy)methyl]toluene (1.29 g, 3.75 mmol).

Step B: To a solution of 2-(o-bromophenyl) ethanol (0.7 g, 3.48 mmol) in DMF (12 mL) was added sodium hydride 60% w/w (209 mg, 5.22 mmol) at 0° C. under N₂. The mixture was stirred at 0° C. for 30 min. Then 2-fluoro-5-iodo-3-[(mesyloxy)methyl]toluene (1.2 g, 3.48 mmol) was added to the mixture. The reaction was allowed to warm to room temperature and was stirred for 2 hours. The reaction mixture was then quenched with saturated aqueous NH₄Cl, diluted with ethyl acetate, washed with water (2×) and the aqueous layer was extracted with ethyl acetate (2×). The combined ethyl acetate layers were washed with brine, dried over Na₂SO₄, filtered and then concentrated in vacuo. The crude product was purified by column chromatography (0-15% ethyl acetate in heptane). to give 3-{[2-(o-bromophenyl)ethoxy]methyl}-2-fluoro-5-iodotoluene (870 mg, 1.94 mmol) as a yellow oil.

Step C: A solution of 3-{[2-(o-bromophenyl)ethoxy]methyl}-2-fluoro-5-iodotoluene (870 mg, 1.94 mmol), tert-butyl carbazate (333 mg, 2.52 mmol), dicaesium carbonate (884 mg, 2.71 mmol), copper iodide (18.4 mg, 96.9 μmol), and 4,5-diazaphenanthrene (69.8 mg, 387 μmol) in DMF (2 mL) was sparged with Ar for 5 min and heated to 80° C. for 3h. Upon cooling to rt, the solution was taken up in EtOAc, filtered through Celite, and washed with brine (3×). The organic phase was dried over Na₂SO₄, filtered, and concentrated. The crude product was purified by silica gel chromatography (5-30% EtOAc/heptane) to give tert-butyl 2-(3-{[2-(o-bromophenyl)ethoxy]methyl}-4-fluoro-5-tolyl)carbazate (496 mg, 1.09 mmol) as yellow oil. m/z (ESI-pos) M+1=399.1 (loss of tBu).

Step D: To a solution of tert-butyl 2-(3-{[2-(o-bromophenyl)ethoxy]methyl}-4-fluoro-5-tolyl)carbazate (496 mg, 1.09 mmol) in DCM (3.7 mL), trifluoroacetic acid (837 μL, 10.9 mmol) was added at ambient temperature. The reaction was stirred for 3 h. The reaction was then quenched with sat. aq. Na₂CO₃ (~20 mL) and extracted with DCM (1×), then washed with water (2×) and brine (1×). The combined aqueous layers were then extracted with 20% IPA/CHCl₃ (2×) and the combined organic layers were dried over Na₂SO₄, filtered, and concentrated to provide (3-{[2-(o-bromophenyl)ethoxy]methyl}-4-fluoro-5-tolyl) hydrazine (386 mg, 1.09 mmol) as a yellow oil which was used without purification in the following step. m/z (ESI-pos) M+2=355.1.

Step E: To a solution of (3-{[2-(o-bromophenyl)ethoxy]methyl}-4-fluoro-5-tolyl) hydrazine (386 mg, 1.09 mmol) in EtOH (4.4 mL) was added N-ethyldiisopropylamine (286 μL, 1.64 mmol) then tert-butyl(2S)-3-cyano-2-methyl-4-oxo-1-piperidinecarboxylate (391 mg, 1.64 mmol) and the mixture was stirred at 80° C. for 24 hours. The mixture was concentrated under reduced pressure to get the crude product, which was purified by column chromatography (0-33% ethyl acetate in heptane) to give tert-butyl (S)-3-amino-2-(3-{[2-(o-bromophenyl)ethoxy]methyl}-4-fluoro-5-tolyl)-4-methyl-1,2,5-triaza-2,4,6,7-tetrahydro-5H-indene-5-carboxylate (130 mg, 227 μmol) as an off white solid m/z (ESI-pos) M+2=575.4.

Step F: To a solution of tert-butyl (S)-3-amino-2-(3-{[2-(0-bromophenyl)ethoxy]methyl}-4-fluoro-5-tolyl)-4-methyl-1,2,5-triaza-2,4,6,7-tetrahydro-5H-indene-5-carboxylate (130 mg, 227 μmol) in tetrahydrofuran (1.1 mL) at 0° C. was added DIEA (118 μL, 680 μmol) and benzenyl chloroformate (56.9 μL, 453 μmol). The mixture was stirred at room temperature for 2 hours and then concentrated to get crude tert-butyl (S)-2-(3-{[2-(o-bromophenyl)ethoxy]methyl}-4-fluoro-5-tolyl)-4-methyl-3-[phenyl(oxycarbonylamino)]-1,2,5-triaza-2,4,6,7-tetrahydro-5H-indene-5-carboxylate (157 mg, 226 μmol) as a yellow solid, which was used directly in next step. m/z (ESI-pos) M+2=695.4.

Step G: To a solution of crude tert-butyl (S)-2-(3-{[2-(o-bromophenyl)ethoxy]methyl}-4-fluoro-5-tolyl)-4-methyl-3-[phenyl(oxycarbonylamino)]-1,2,5-triaza-2,4,6,7-tetrahydro-5H-indene-5-carboxylate (140 mg, 202 μmol) in pyridine (0.7 mL) was added 2,2-dimethoxyethylamine (33 μL, 303 μmol). The mixture was stirred at room temperature for 1 hour and then concentrated under vacuum. The obtained residue was purified by column chromatography (0-100% ethyl acetate in heptane) to give tert-butyl (S)-2-(3-{[2-(o-bromophenyl)ethoxy]methyl}-4-fluoro-5-tolyl)-3-[3-(2,2-dimethoxyethyl)ureido]-4-methyl-1,2,5-triaza-2,4,6,7-tetrahydro-5H-indene-5-carboxylate (78 mg, 111 μmol) as a white solid. m/z (ESI-pos) M+2=706.4.

Step H: To a solution of tert-butyl (S)-2-(3-{[2-(o-bromophenyl)ethoxy]methyl}-4-fluoro-5-tolyl)-3-[3-(2,2-dimethoxyethyl)ureido]-4-methyl-1,2,5-triaza-2,4,6,7-tetrahydro-5H-indene-5-carboxylate (78 mg, 111 μmol) in tetrahydrofuran (553 μL) was added methanesulfonic acid (21.6 μL, 332 μmol). The mixture was stirred at 60° C. for 1 hour and then cooled to room temperature. The reaction mixture was diluted with H₂O and extracted with ethyl acetate (2×). The combined organic layers were dried over anhydrous Na₂SO₄ and concentrated under vacuum to get the crude product, which was purified by column chromatography (0-100% ethyl acetate in heptane) to give tert-butyl (S)-2-(3-{[2-(0-bromophenyl)ethoxy]methyl}-4-fluoro-5-tolyl)-4-methyl-3-(2-oxo-2,3-dihydro-1H-imidazol-1-yl)-1,2,5-triaza-2,4,6,7-tetrahydro-5H-indene-5-carboxylate (33.4 mg, 52.1 μmol) as a white solid. m/z (ESI-pos) M+2=642.4.

Step I: To a solution tert-butyl (S)-2-(3-{[2-(o-bromophenyl)ethoxy]methyl}-4-fluoro-5-tolyl)-4-methyl-3-(2-oxo-2,3-dihydro-1H-imidazol-1-yl)-1,2,5-triaza-2,4,6,7-tetrahydro-5H-indene-5-carboxylate (33.4 mg, 52.1 μmol) in NMP (1.6 mL) was added copper iodide (9.93 mg, 52.1 μmol), (1R,2R)-1,2-bis(methylamino)cyclohexane (9.87 μL, 62.6 μmol) and dipotassium carbonate (22 mg, 156 μmol). The mixture was stirred at 130° C. for 4 hours and then cooled to room temperature, diluted with H₂O and extracted with ethyl acetate (2×). The combined ethyl acetate layers were washed with aqueous LiCl (2×), then dried over Na₂SO₄, filtered and concentrated in vacuo. The crude product was purified by column chromatography (0-80% ethyl acetate in heptane) to give tert-butyl (S)-26-fluoro-8,27-dimethyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(28),3,9,12,15(20),16,18,25(29),26-nonaene-7-carboxylate (14.5 mg, 25.9 μmol). m/z (ESI-pos) M+1=560.4.

Step J: Tert-butyl (S)-26-fluoro-8,27-dimethyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (14.5 mg, 25.9 μmol) in HCl/1,4-dioxane (4M, 0.5 mL) was stirred at room temperature for 1 hour. The mixture was concentrated under vacuum to give crude (S)-26-fluoro-8,27-dimethyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (12.9 mg, 26 μmol) as a white solid. m/z (ESI-pos) M+1=460.3.

Step K: To a solution of (S)-26-fluoro-8,27-dimethyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (12.9 mg, 26 μmol) in DMF (0.5 mL) was added 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (14 mg, 34.1 μmol) and DIEA (22.8 μL, 131 μmol) and the mixture was stirred at room temperature for 5 minutes, then HATU (20 mg, 52.4 μmol) was added and the mixture was stirred at room temperature for 16 hours. Then H₂O was added and extracted with ethyl acetate (2×). The combined ethyl acetate layers were washed with brine, then dried over Na₂SO₄ and concentrated in vacuo to get the crude product. The crude reaction mixture directly loaded onto the reverse phase column and purified by Prep HPLC (Isco ACCQ Prep, Gemini column NX-C18, 250×21 mm, 20 ml/min, 45 minute separation, 20 to 80% H₂O/ACN with 0.1% Formic acid buffer), pure fractions were collected and lyophilized to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8,27-dimethyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (11 mg, 12.9 μmol). m/z (ESI-pos) M+1=853.6; 1H NMR (400 MHz, CHLOROFORM-D) δ 11.26 (s, 1H), 7.62-7.37 (m, 3H), 7.37-7.26 (m, 4H), 7.24-6.88 (m, 2H), 6.77-6.62 (m, 1H), 6.50-6.05 (m, 2H), 5.92-5.19 (m, 1H), 4.91-4.69 (m, 1H), 4.63-4.40 (m, 2H), 3.92-3.76 (m, 2H), 3.69-2.74 (m, 8H), 2.30 (s, 3H), 2.00-1.62 (m, 4H), 1.55-0.97 (m, 15H).

### Procedure for Synthesis of Compound 141

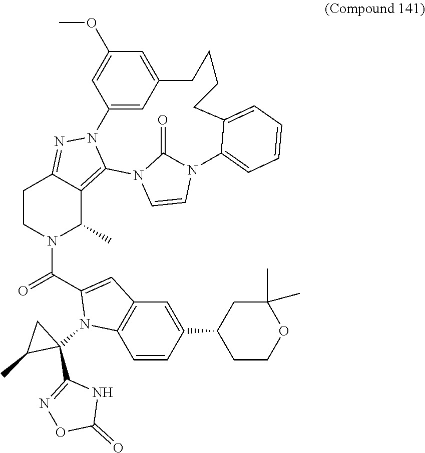

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-27-methoxy-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 141 was synthesized following the procedure of Example 5 & 6. Tert-butyl (S)-3-amino-2-(3-bromophenyl)-4-methyl-2,4,6,7-tetrahydro-5H-pyrazolo[4,3-c]pyridine-5-carboxylate being replaced with tert-butyl (S)-2-(3-allyl-5-methoxyphenyl)-3-amino-4-methyl-1,2,5-triaza-2,4,6,7-tetrahydro-5H-indene-5-carboxylate in example 5 step A. m/z (ESI-pos) M+1=849.7 and 850.6; 1H NMR @398K (400 MHz, TETRACHLOROETHAN) δ 11.11 (s, 1H), 7.69-7.49 (m, 2H), 7.45-7.21 (m, 5H), 7.13 (s, 1H), 6.88-6.65 (m, 3H), 6.54-6.15 (m, 2H), 5.77 (s, 1H), 4.59 (s, 1H), 3.99-3.79 (m, 5H), 3.72-3.59 (m, 1H), 3.22 (d, J=13.7 Hz, 1H), 3.15-3.01 (m, 2H), 3.02-2.86 (m, 1H), 2.76 (dd, J=16.0, 6.2 Hz, 2H), 2.61-2.38 (m, 1H), 2.04-1.53 (m, 12H), 1.50-1.38 (m, 6H), 1.34 (d, J=3.8 Hz, 3H), 1.26 (s, 3H).

### Example 142—Synthesis of Compound 142

### Synthetic Scheme

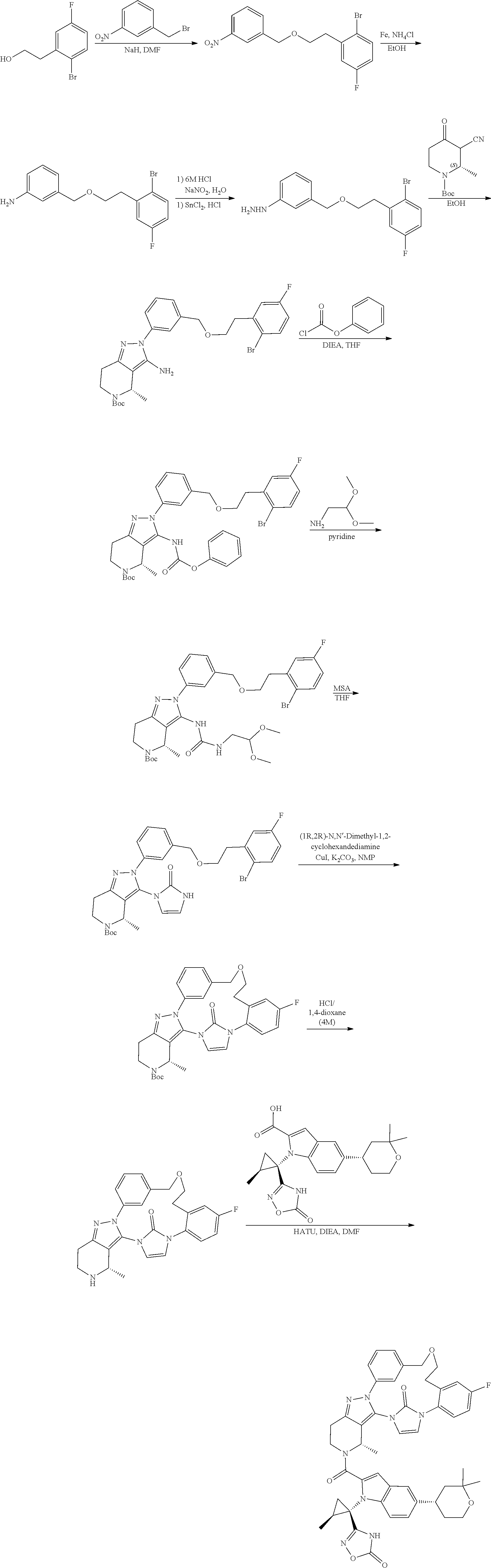

### Procedure for Synthesis of Compound 142

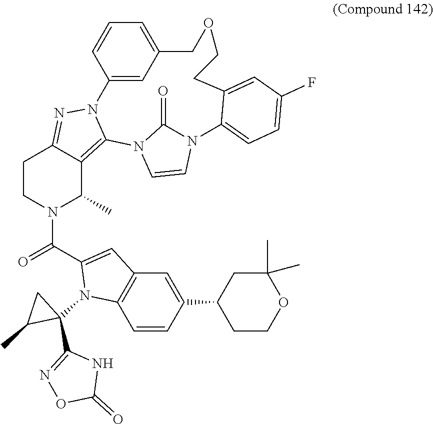

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To the solution of 2-(2-bromo-5-fluorophenyl) ethanol (1.5 g, 6.85 mmol) in DMF (13.7 mL) was added sodium hydride 60% w/w (548 mg, 13.7 mmol) at 0° C. under N₂ and stirred for 30 min. Then m-(bromomethyl) nitrobenzene (1.63 g, 7.53 mmol) was added to the mixture. The reaction was allowed to warm to room temperature and stirred for 1 h. The reaction was quenched with saturated aqueous NH₄Cl, diluted with ethyl acetate, and washed with water (3×). The aqueous layer was extracted with ethyl acetate (3×). The combined organic layers were washed with brine (3×), dried over anhydrous Na₂SO₄, filtered, and evaporated under reduced pressure. The crude compound was purified by column chromatography (0-15% EtOAc in heptane) to give 1-bromo-4-fluoro-2-{2-[(m-nitrophenyl)methoxy]ethyl}benzene (2.3 g, 6.49 mmol) as a yellow solid. m/z (ESI-pos)=not found-does not ionize

Step B: To the solution of 1-bromo-4-fluoro-2-{2-[(m-nitrophenyl)methoxy]ethyl}benzene (2.3 g, 6.49 mmol) in EtOH (25.5 mL) and water (8.1 mL), iron (1.81 g, 32.5 mmol) and ammonium chloride (1.74 g, 32.5 mmol) were added under N₂. The reaction stirred at 70° C. overnight. The reaction mixture was cooled to ambient temperature and filtered through Celite. The filtrate was diluted with ethyl acetate, washed with water (3×) and the aqueous layer was extracted with ethyl acetate (3×). The combined organic layers were washed with brine, dried over anhydrous Na₂SO₄, filtered and concentrated. The crude compound was purified by column chromatography (0-30% EtOAc in heptane) to give m-{[2-(2-bromo-5-fluorophenyl)ethoxy]methyl}aniline (1.89 g, 5.83 mmol) as a slightly yellow oil. m/z (ESI-pos) M+1=324.1/326.1

Step C: A solution of m-{[2-(2-bromo-5-fluorophenyl)ethoxy]methyl}aniline (1.89 g, 5.83 mmol) in conc. HCl (15 mL) and H₂O (15 mL) was cooled to 0° C. and sodium nitrite (442 mg, 6.41 mmol) in H₂O (5 mL) was added dropwise. The mixture solution was stirred at 0° C. for 1 h and then dichlorostannanediyl (2.43 g, 12.8 mmol) in conc. HCl (15 mL) and H₂O (15 mL) was added dropwise. The resulting mixture solution was allowed to warm to room temperature and stirred for an additional 45 min. The reaction mixture was diluted with water (200 mL) and adjusted to pH ~10 with 5 N aq. NaOH. The resulting suspension was extracted with ethyl acetate (3×). The combined organic layers were washed with brine and dried over anhydrous Na₂SO₄, filtered and evaporated under reduced pressure. The resulting crude compound (m-{[2-(2-bromo-5-fluorophenyl)ethoxy]methyl}phenyl)hydrazine (1.85 g, 5.45 mmol) was used in the following step without purification. m/z (ESI-pos) M+1=339.1/341.1

Step D: To a solution of (m-{[2-(2-bromo-5-fluorophenyl)ethoxy]methyl}phenyl)hydrazine (1.85 g, 5.45 mmol) in EtOH (16.5 mL) was added N-ethyldiisopropylamine (1.29 mL, 7.43 mmol) and tert-butyl(2S)-3-cyano-2-methyl-4-oxo-1-piperidinecarboxylate (1.18 g, 4.95 mmol) at room temperature. The mixture was stirred at 80° C. overnight. The reaction mixture was cooled down to room temperature, diluted with ethyl acetate and washed with water (3×). The aqueous layer was extracted with ethyl acetate (2×). The combined organic layers were washed with brine, dried over anhydrous Na₂SO₄, filtered and the filtrate was evaporated under reduced pressure. The crude compound was purified by column chromatography (10-60% EtOAc in heptane) to give tert-butyl (S)-3-amino-2-(m-{[2-(2-bromo-5-fluorophenyl)ethoxy]methyl}phenyl)-4-methyl-1,2,5-triaza-2,4,6,7-tetrahydro-5H-indene-5-carboxylate (1.26 g, 2.25 mmol) as a yellow solid. m/z (ESI-pos) M+1=559.3/561.3

Step E: To a solution of tert-butyl (S)-3-amino-2-(m-{[2-(2-bromo-5-fluorophenyl)ethoxy]methyl}phenyl)-4-methyl-1,2,5-triaza-2,4,6,7-tetrahydro-5H-indene-5-carboxylate (435 mg, 778 μmol) in THF (3.89 mL) was added N-ethyldiisopropylamine (406 μL, 2.33 mmol) followed by benzenyl chloroformate (195 μL, 1.56 mmol). The reaction mixture was stirred at 25° C. for 30 min. The reaction was then directly concentrated under reduced pressure to give the crude tert-butyl (S)-2-(m-{[2-(2-bromo-5-fluorophenyl)ethoxy]methyl}phenyl)-4-methyl-3-[phenyl(oxycarbonylamino)]-1,2,5-triaza-2,4,6,7-tetrahydro-5H-indene-5-carboxylate (528 mg, 777 μmol) as a yellow oil, which was used directly in the next step without any further purification. m/z (ESI-pos) M+1=679.3/681.3

Step F Crude tert-butyl (S)-2-(m-{[2-(2-bromo-5-fluorophenyl)ethoxy]methyl}phenyl)-4-methyl-3-[phenyl(oxycarbonylamino)]-1,2,5-triaza-2,4,6,7-tetrahydro-5H-indene-5-carboxylate (528 mg, 777 μmol) was dissolved in pyridine (2.59 mL) and then 2,2-dimethoxyethylamine (127 μL, 1.17 mmol) was added. The mixture was stirred at room temperature for 1 h and directly concentrated under reduced pressure to give the crude residue as a yellow oil (537 mg, 777 μmol) which was used in the following step without purification. m/z (ESI-pos) M+1=690.4/692.4

Step G: To a solution of tert-butyl (S)-2-(m-{[2-(2-bromo-5-fluorophenyl)ethoxy]methyl}phenyl)-3-[3-(2,2-dimethoxyethyl)ureido]-4-methyl-1,2,5-triaza-2,4,6,7-tetrahydro-5H-indene-5-carboxylate (537 mg, 778 μmol) in THF (5.2 mL) was added methanesulfonic acid (252 μL, 3.89 mmol). The mixture was stirred at 55° C. for 1.5 h and then cooled to ambient temperature. Saturated aqueous NaHCO₃ was added until pH 8 then tert-butyl-tert-butyl(oxycarbonyloxy) formylate (255 mg, 1.17 mmol) was added, and the resulting mixture was stirred at room temperature for 1 h. Upon completion, the reaction mixture was diluted with water and ethyl acetate. The aqueous layer was extracted with ethyl acetate (2×), and the combined organic layers were washed with brine and dried over anhydrous Na₂SO₄, filtered and the filtrate was evaporated under reduced pressure. The crude compound was purified by column chromatography (5-60% EtOAc in heptane) to give tert-butyl (S)-2-(m-{[2-(2-bromo-5-fluorophenyl)ethoxy]methyl}phenyl)-4-methyl-3-(2-oxo-2,3-dihydro-1H-imidazol-1-yl)-1,2,5-triaza-2,4,6,7-tetrahydro-5H-indene-5-carboxylate (265 mg, 423 μmol) as a white solid. m/z (ESI-pos) M+1=626.3/628.3

Step H: A solution of tert-butyl (S)-2-(m-{[2-(2-bromo-5-fluorophenyl)ethoxy]methyl}phenyl)-4-methyl-3-(2-oxo-2,3-dihydro-1H-imidazol-1-yl)-1,2,5-triaza-2,4,6,7-tetrahydro-5H-indene-5-carboxylate (120 mg, 192 μmol), CuI (29.2 mg, 153 μmol), (1S,2S)-1,2-bis(methylamino)cyclohexane (27.2 mg, 192 μmol), and K₂CO₃ (79.4 mg, 575 μmol) in NMP (3.5 mL) was sparged with Ar and then heated to 130° C. for 4 h then cooled to ambient temperature, diluted with ethyl acetate, and washed with water (3×). The aqueous layer was extracted with ethyl acetate (3×), and the combined organic layers were washed with brine, dried over anhydrous Na₂SO₄, filtered and evaporated under reduced pressure. The crude compound was purified by column chromatography (10-50% EtOAc in heptane) to give tert-butyl (S)-18-fluoro-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (32.5 mg, 59.6 μmol) as a white solid. m/z (ESI-pos) M+1=546.4

Step I: HCl (1.5 mL; 4N in dioxane) was added to a vial containing tert-butyl (S)-18-fluoro-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (32.5 mg, 59.6 μmol) and the solution stirred at room temperature for 20 min. Upon completion, the solvent was evaporated to give (S)-18-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (24 mg, 49.8 μmol) as a white solid which was used in the next step without further purification. m/z (ESI-pos) M+1=446.3

Step J: To a solution of (S)-18-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (24 mg, 49.8 μmol) in DMF (1 mL) was added 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (64.7 μmol), N-ethyldiisopropylamine (43 μL, 249 μmol), and HATU (37.9 mg, 99.6 μmol). The mixture stirred at room temperature overnight. The reaction was then diluted with ethyl acetate and washed with sat. aq. NH₄Cl then water. The aqueous layer was extracted with ethyl acetate (3×), and the combined organic layers were washed with brine, dried over anhydrous Na₂SO₄, filtered and the filtrate was evaporated under reduced pressure. The crude compound purified by column chromatography (0-4% MeOH in DCM) to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (22.5 mg, 26.8 μmol). m/z (ESI-pos) M+1=839.6; ¹H NMR (400 MHz, CHLOROFORM-D) δ 11.26 (d, J=21.4 Hz, 1H), 7.61-7.43 (m, 4H), 7.35-7.12 (m, 3H), 7.07-6.86 (m, 2H), 6.79-6.64 (m, 1H), 6.48-5.91 (m, 2H), 5.83 (dq, J=34.2, 6.7 Hz, 1H), 4.77-4.40 (m, 3H), 3.84 (dq, J=13.4, 7.0 Hz, 2H), 3.69-3.54 (m, 1H), 3.46-3.09 (m, 3H), 3.08-2.96 (m, 2H), 2.92-2.80 (m, 1H), 1.96-1.56 (m, 8H), 1.36-1.01 (m, 13H).

### Example 143—Synthesis of Compound 143

### Synthetic Scheme

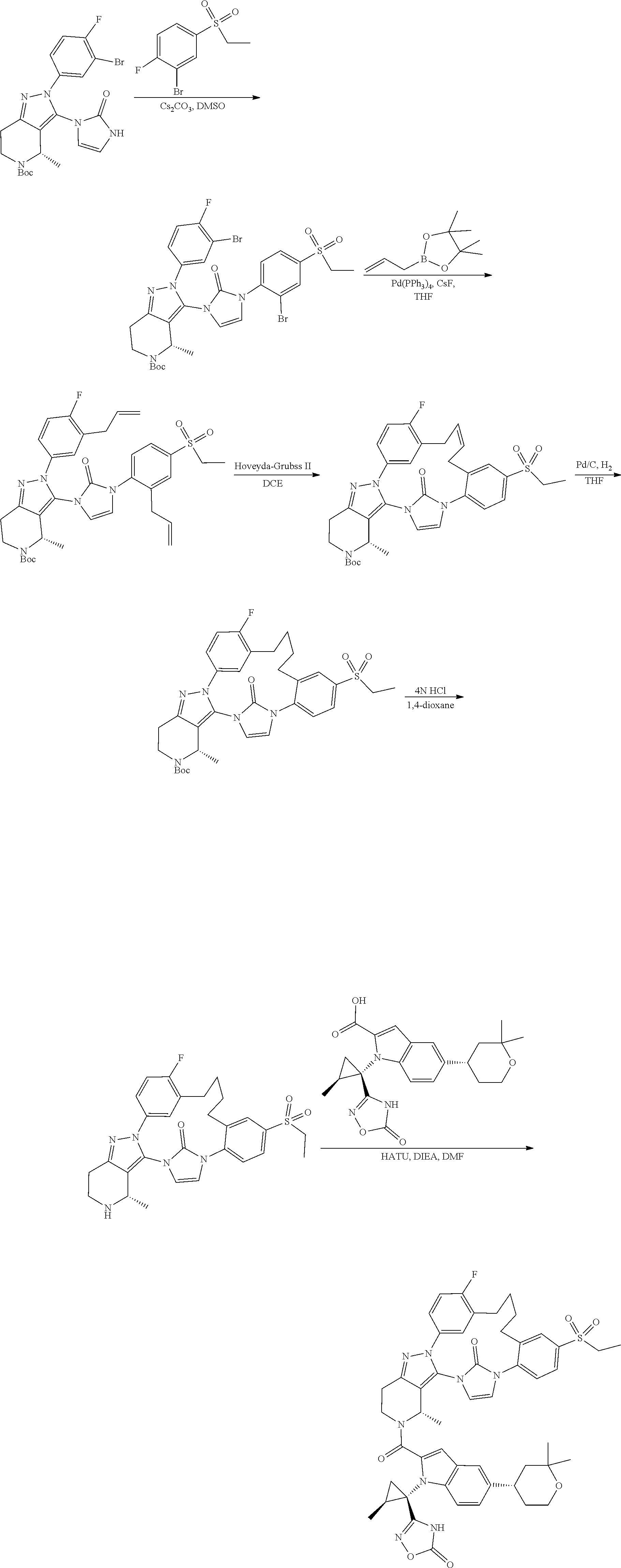

### Procedure for Synthesis of Compound 143

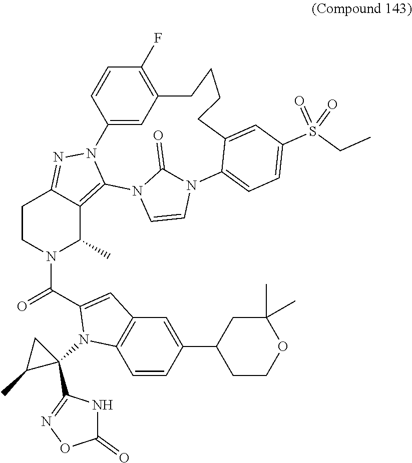 [SMILES: CCS(=O)(=O)c1ccc2c(c1)CCCCc1cc(ccc1F)-n1nc3c(c1-n1ccn-2c1=O)[C@H](C)N(C(=O)c1cc2cc(C4CCOC(C)(C)C4)ccc2n1[C@@]1(c2noc(=O)[nH]2)C[C@@H]1C)CC3]

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18-(ethylsulfonyl)-26-fluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a solution of tert-butyl (S)-2-(3-bromo-4-fluorophenyl)-4-methyl-3-(2-oxo-2,3-dihydro-1H-imidazol-1-yl)-1,2,5-triaza-2,4,6,7-tetrahydro-5H-indene-5-carboxylate (64 mg, 130 μmol) in DMSO (900 μL) was added dicaesium carbonate (84.7 mg, 260 μmol). The mixture was stirred at room temperature for 5 min, followed by the addition of 2-bromo-4-(ethylsulfonyl)-1-fluorobenzene (104 mg, 390 μmol). The reaction was heated at 40° C. for 45 min. Upon completion, the reaction was cooled to room temperature and quenched by the addition of H₂O and saturated aqueous NH₄Cl. The quenched reaction mixture was extracted with EtOAc (3×). The combined organic layers were washed with brine, then dried over Na₂SO₄, filtered and concentrated in vacuo. The residue was purified by column chromatography (20-70% EtOAc in heptane), to give tert-butyl (S)-3-{3-[2-bromo-4-(ethylsulfonyl)phenyl]-2-oxo-2,3-dihydro-1H-imidazol-1-yl}-2-(3-bromo-4-fluorophenyl)-4-methyl-1,2,5-triaza-2,4,6,7-tetrahydro-5H-indene-5-carboxylate (79.5 mg, 108 μmol) as a white solid. m/z (ESI-pos) M+1=740.2/742.2

Step B: Tert-butyl (S)-3-{3-[2-bromo-4-(ethylsulfonyl)phenyl]-2-oxo-2,3-dihydro-1H-imidazol-1-yl}-2-(3-bromo-4-fluorophenyl)-4-methyl-1,2,5-triaza-2,4,6,7-tetrahydro-5H-indene-5-carboxylate (79.5 mg, 108 μmol), bis[2-(diphenylphosphino)-2,4-cyclopentadien-1-ide]-dichloro-palladamethane-dichloromethane-iron (1/1/1/1) (8.78 mg, 10.8 μmol), and dipotassium carbonate (44.6 mg, 323 μmol) were dissolved in 1,4-dioxane (700 μL) and H₂O (110 μL) and 2-allyl-4,4,5,5-tetramethyl-1,3,2-dioxaborolane (121 μL, 645 μmol) was added. The resulting solution was degassed with Ar for 5 min then heated to 80° C. After 1.5h, another batch of 2-allyl-4,4,5,5-tetramethyl-1,3,2-dioxaborolane (121 μL, 645 μmol) was added and stirred for an additional 3 h at 80° C. After cooling to room temperature, the reaction was concentrated and the crude material was purified by column chromatography (10-80% EtOAc in heptane) to give tert-butyl (S)-3-{3-[2-allyl-4-(ethylsulfonyl)phenyl]-2-oxo-2,3-dihydro-1H-imidazol-1-yl}-2-(3-allyl-4-fluorophenyl)-4-methyl-1,2,5-triaza-2,4,6,7-tetrahydro-5H-indene-5-carboxylate (48.6 mg, 73.4 μmol) as a white solid. m/z (ESI-pos) M+1=662.4

Step C: Tert-butyl (S)-3-[3-(2-allyl-4-chlorophenyl)-2-oxo-2,3-dihydro-1H-imidazol-1-yl]-2-(3-allyl-4-fluorophenyl)-4-methyl-1,2,5-triaza-2,4,6,7-tetrahydro-5H-indene-5-carboxylate (50 mg, 82.8 μmol) was dissolved in DCE (41.4 mL) and acetic acid (5.68 μL, 99.3 μmol) was added followed by ruthenium o-isopropoxymethanediidyltoluene 1,3-bis(mesityl)-2,2-imidazolidinediide dichloride (13 mg, 20.7 μmol). The solution was degassed with Ar for 10 min and the reaction was heated to 40° C. where it stirred for 2.5 h. The reaction was cooled to room temperature and concentrated. The crude material was purified by silica gel chromatography (10-80% EtOAc in heptane) to give tert-butyl (S)-18-chloro-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (30.3 mg, 52.6 μmol) as an off-white solid. m/z (ESI-pos) M+1=634.4

Step D: To a solution of tert-butyl (S)-18-(ethylsulfonyl)-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (44.5 mg, 70.2 μmol) in EtOAc (1.4 mL) was added Pd 10% w/w (14.9 mg, 14 μmol) and the mixture was then purged with H₂ gas and the mixture was allowed to stir under a H₂ atmosphere with balloon pressure at ambient temperature. After 2 h, the mixture was filtered through a nylon filter, the filter was washed with EtOAc and the filtrate was concentrated. The crude material was used in the following step without purification. m/z (ESI-pos) M+1=636.4

Step E: 4N hydrogen chloride (1.5 mL) was added to a vial containing tert-butyl (S)-18-(ethylsulfonyl)-26-fluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (44.6 mg, 70.2 μmol) and the solution stirred at room temperature for 20 min. Upon completion, the solvent was evaporated to give (S)-18-(ethylsulfonyl)-26-fluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (36 mg, 62.9 μmol)) as a white solid which was used in the next step without further purification. m/z (ESI-pos) M+1=536.4

Step F: To a solution of (S)-18-(ethylsulfonyl)-26-fluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (36 mg, 62.9 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (33.7 mg, 81.8 μmol) in DMF (1.26 mL) was added N-ethyldiisopropylamine (55 μL, 315 μmol) and HATU (47.9 mg, 126 μmol). The mixture stirred at room temperature overnight. The reaction solution was directly purified by Prep HPLC (Isco ACCQ Prep, Gemini column NX-C18, 250×21 mm, 20 mL/min, 55 minute separation, 20 to 80% H₂O/ACN with 0.1% Formic acid buffer), pure fractions were collected and lyophilized to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18-(ethylsulfonyl)-26-fluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (34.8 mg, 37.5 μmol) as a white solid. m/z (ESI-pos) M+1=929.6; ¹H NMR (400 MHz, CHLOROFORM-D) δ 11.24 (s, 1H), 7.91-7.71 (m, 3H), 7.62-7.34 (m, 5H), 7.30-7.07 (m, 2H), 6.76-6.66 (m, 1H), 6.42 (dd, J=6.4, 3.1 Hz, 1H), 6.35-5.96 (m, 1H), 5.86-5.71 (m, 1H), 5.24-4.41 (m, 1H), 3.84 (tdd, J=11.7, 7.9, 4.6 Hz, 3H), 3.69-3.51 (m, 1H), 3.43-3.22 (m, 1H), 3.19-2.86 (m, 5H), 2.67-2.43 (m, 1H), 1.93-1.87 (m, 1H), 1.86-1.48 (m, 10H), 1.44-1.20 (m, 12H), 1.21-0.99 (m, 3H).

### Example 144—Synthesis of Compound 144

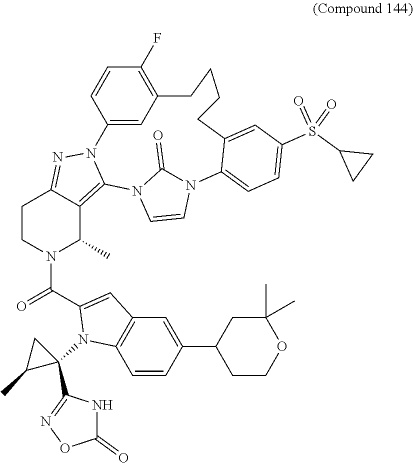 [SMILES: C[C@H]1c2c(nn3c2-n2ccn(c2=O)-c2ccc(S(=O)(=O)C4CC4)cc2CCCCc2cc-3ccc2F)CCN1C(=O)c1cc2cc(C3CCOC(C)(C)C3)ccc2n1[C@@]1(c2noc(=O)[nH]2)C[C@@H]1C]

### (S)-18-(cyclopropylsulfonyl)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 144 was synthesized following the procedure of Example 143, with 2-bromo-4-(ethylsulfonyl)-1-fluorobenzene being replaced with 2-bromo-4-(cyclopropylsulfonyl)-1-fluorobenzene in step A. m/z (ESI-pos) M+1=941.6; ¹H NMR (400 MHz, CHLOROFORM-D) δ 11.23 (d, J=12.8 Hz, 1H), 7.90-7.81 (m, 2H), 7.62-7.32 (m, 5H), 7.27-7.25 (m, 1H), 7.21-7.09 (m, 1H), 6.76-6.66 (m, 1H), 6.45-5.95 (m, 2H), 5.79 (dd, J=26.0, 6.8 Hz, 1H), 5.23-4.42 (m, 1H), 3.92-3.30 (m, 4H), 3.20-2.90 (m, 3H), 2.72-2.40 (m, 1H), 1.94-1.48 (m, 14H), 1.43-1.23 (m, 9H), 1.21-1.00 (m, 7H).

### Example 145—Synthesis of Compound 145

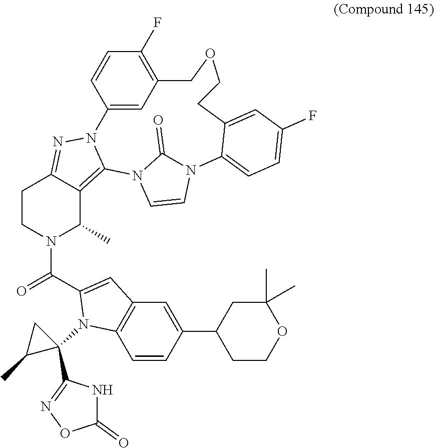 [SMILES: C[C@H]1c2c(nn3c2-n2ccn(c2=O)-c2ccc(F)cc2CCOCc2cc-3ccc2F)CCN1C(=O)c1cc2cc(C3CCOC(C)(C)C3)ccc2n1[C@@]1(c2noc(=O)[nH]2)C[C@@H]1C]

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18,26-difluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 145 was synthesized following the procedure of Example 142, with m-(bromomethyl) nitrobenzene being replaced with 2-(bromomethyl)-1-fluoro-4-nitrobenzene in step A. m/z (ESI-pos) M+1=857.5; ¹H NMR (400 MHz, CHLOROFORM-D) δ 11.25 (s, 1H), 7.62-7.42 (m, 4H), 7.25-7.14 (m, 2H), 7.09-6.95 (m, 2H), 6.78-6.63 (m, 1H), 6.46-6.05 (m, 2H), 5.93-5.75 (m, 1H), 5.28-4.40 (m, 4H), 3.84 (d, J=10.5 Hz, 3H), 3.70-3.53 (m, 1H), 3.54-2.69 (m, 5H), 1.90 (t, J=5.3 Hz, 1H), 1.84-1.46 (m, 6H), 1.39-1.01 (m, 13H).

### Example 146—Synthesis of Compound 146

### Synthetic Scheme

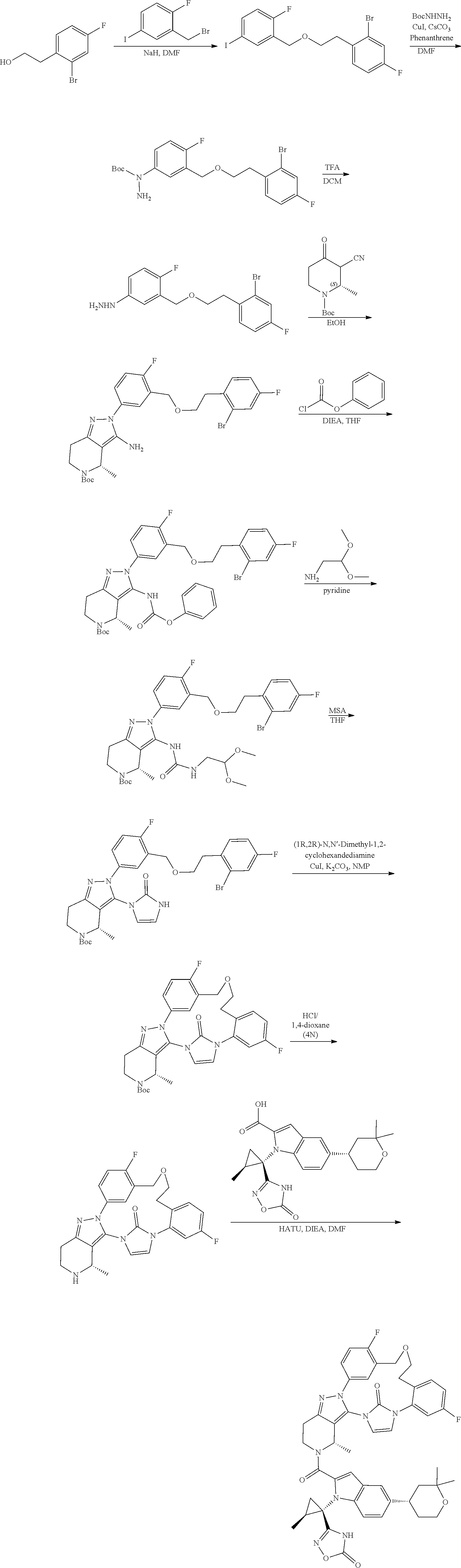

### Procedure for Synthesis of Compound 146

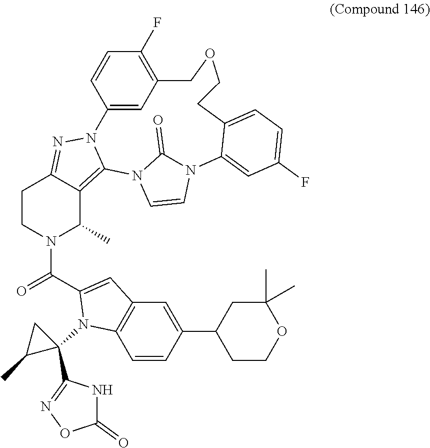 [SMILES: C[C@H]1c2c(nn3c2-n2ccn(c2=O)-c2cc(F)ccc2CCOCc2cc-3ccc2F)CCN1C(=O)c1cc2cc(C3CCOC(C)(C)C3)ccc2n1[C@@]1(c2noc(=O)[nH]2)C[C@@H]1C]

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,26-difluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a solution of 2-(2-bromo-4-fluorophenyl) ethanol (0.5 g, 2.28 mmol) and 2-(bromomethyl)-1-fluoro-4-iodobenzene (791 mg, 2.51 mmol) in DMF (4.57 mL) was added sodium hydride 60% w/w (95.9 mg, 2.4 mmol) at 0° C. The reaction was stirred at 0° C. for 30 min and then at room temperature for 1.5 h. The reaction mixture was quenched with sat. aq. NH₄Cl (2 mL), diluted with ethyl acetate (20 mL), and washed with brine (2×20 mL). The organic layer was dried over Na₂SO₄ and concentrated in vacuo. The crude product was purified by column chromatography (0-10% EtOAc in heptane) to give 2-bromo-4-fluoro-1-{2-[(2-fluoro-5-iodophenyl)methoxy]ethyl}benzene (860 mg, 1.9 mmol) as a clear oil. m/z (ESI-pos) M+1=not found-does not ionize

Step B: A solution of 2-bromo-4-fluoro-1-{2-[(2-fluoro-5-iodophenyl)methoxy]ethyl}benzene (860 mg, 1.9 mmol), tert-butyl carbazate (301 mg, 2.28 mmol), copper iodide (18.1 mg, 94.9 μmol), 4,5-diazaphenanthrene (68.4 mg, 380 μmol), and dicaesium carbonate (680 mg, 2.09 mmol) in DMF (1.9 mL) was sparged with Ar for 5 min and heated to 80° C. for 3 h. The mixture was cooled to room temperature then diluted with EtOAc (20 mL) and washed with brine (3×20 mL). The organics were dried over Na₂SO₄, filtered, and concentrated. The crude product was purified by silica gel chromatography (5-30% EtOAc in heptane) to give tert-butyl 2-(3-{[2-(2-bromo-4-fluorophenyl)ethoxy]methyl}-4-fluorophenyl)carbazate (570 mg, 1.25 mmol). m/z (ESI-pos) M+1-tert-butyl=401.1/403.1

Step C: To a solution of tert-butyl 2-(3-{[2-(2-bromo-4-fluorophenyl)ethoxy]methyl}-4-fluorophenyl)carbazate (570 mg, 1.25 mmol) in DCM (12.5 mL) was added trifluoroacetic acid (1.86 mL, 24.3 mmol). The reaction was stirred at room temperature for 2.5 h. Upon completion, the reaction was quenched with sat. aq. Na₂CO₃ (20 mL). The organic layer was separated, washed with sat. aq. NaHCO₃ (3×15 mL), dried over Na₂SO₄, filtered, and concentrated in vacuo to provide crude (3-{[2-(2-bromo-4-fluorophenyl)ethoxy]methyl}-4-fluorophenyl)hydrazine (assumed quant. yield; 445 mg, 1.25 mmol) which was taken to the next step without purification. m/z (ESI-pos) M+1=357.1/359.1

Step D: To a solution of crude (3-{[2-(2-bromo-4-fluorophenyl)ethoxy]methyl}-4-fluorophenyl)hydrazine (445 mg, 1.25 mmol) in EtOH (5 mL) was added N-ethyldiisopropylamine (326 μL, 1.87 mmol) then tert-butyl(2S)-3-cyano-2-methyl-4-oxo-1-piperidinecarboxylate (327 mg, 1.37 mmol). The mixture was stirred at 80° C. for 36 h. After completion, the reaction mixture was cooled to room temperature, the volatiles were evaporated under reduced pressure. The crude mixture was purified by column chromatography (5-60% EtOAc in heptane) to give tert-butyl (S)-3-amino-2-(3-{[2-(2-bromo-4-fluorophenyl)ethoxy]methyl}-4-fluorophenyl)-4-methyl-1,2,5-triaza-2,4,6,7-tetrahydro-5H-indene-5-carboxylate (295 mg, 511 μmol) as a yellow oil over 2 steps. m/z (ESI-pos) M+1=577.3/579.3

Step E: To a solution of tert-butyl (S)-3-amino-2-(3-{[2-(2-bromo-4-fluorophenyl)ethoxy]methyl}-4-fluorophenyl)-4-methyl-1,2,5-triaza-2,4,6,7-tetrahydro-5H-indene-5-carboxylate (295 mg, 511 μmol) in THF (2.55 mL, 31.4 mmol) was added N-ethyldiisopropylamine (267 μL, 1.53 mmol) followed by benzenyl chloroformate (128 μL, 1.02 mmol). The reaction mixture was stirred at 25° C. for 30 min and then directly concentrated under reduced pressure to give crude tert-butyl (S)-2-(3-{[2-(2-bromo-4-fluorophenyl)ethoxy]methyl}-4-fluorophenyl)-4-methyl-3-[phenyl(oxycarbonylamino)]-1,2,5-triaza-2,4,6,7-tetrahydro-5H-indene-5-carboxylate (356 mg, 510 μmol), which was used directly in the next step without any further purification. m/z (ESI-pos) M+1=697.3/699.3

Step F: Crude tert-butyl (S)-2-(3-{[2-(2-bromo-4-fluorophenyl)ethoxy]methyl}-4-fluorophenyl)-4-methyl-3-[phenyl(oxycarbonylamino)]-1,2,5-triaza-2,4,6,7-tetrahydro-5H-indene-5-carboxylate (356 mg, 510 μmol) was dissolved in pyridine (1.7 mL) and then 2,2-dimethoxyethylamine (83.4 μL, 766 μmol) was added. The reaction stirred at room temperature overnight and then directly concentrated. The crude residue was purified by column chromatography (5-80% EtOAc in heptane) to give tert-butyl (S)-2-(3-{[2-(2-bromo-4-fluorophenyl)ethoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (205 mg, 289 μmol) as a yellow foam over two steps. m/z (ESI-pos) M+1=708.3/710.3

Step G: To a solution of tert-butyl (S)-2-(3-{[2-(2-bromo-4-fluorophenyl)ethoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (205 mg, 289 μmol) in THF (1.93 mL) was added methanesulfonic acid (94 μL, 1.45 mmol). The mixture was stirred at 55° C. for 30 min and then cooled to ambient temperature. Saturated aqueous NaHCO₃ was added until pH 8 then tert-butyl-tert-butyl(oxycarbonyloxy) formylate (69.5 mg, 318 μmol) was added and the resulting mixture was stirred at room temperature for an additional 30 min. The reaction mixture was diluted with water (10 mL) and was extracted with ethyl acetate (10 mL×2). The combined organic layers were washed with brine (20 mL) and dried over anhydrous Na₂SO₄, filtered and concentrated under reduced pressure. The crude compound was purified by column chromatography (5-90% EtOAc in heptane) to give tert-butyl (S)-2-(3-{[2-(2-bromo-4-fluorophenyl)ethoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (162 mg, 251 μmol) as a white solid. m/z (ESI-pos) M+1=644.4/646.3

Step H: A solution of tert-butyl (S)-2-(3-{[2-(2-bromo-4-fluorophenyl)ethoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (80 mg, 124 μmol), CuI (23.6 mg, 124 μmol), rac-(1R,2R)-1,2-bis(methylamino)cyclohexane (29.4 μL, 186 μmol), and K₂CO₃ (51.5 mg, 372 μmol) in NMP (4.14 mL) was sparged with Ar and then heated to 130° C. for 5 h. The solution was then cooled to ambient temperature and diluted with water. The mixture was then extracted with EtOAc (3×10 mL). The combined organic layers were washed with brine (2×), dried over Na₂SO₄, filtered and concentrated. The crude material was then purified via column chromatography (10-50% EtOAc in heptane) to give tert-butyl (S)-17,26-difluoro-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (25.2 mg, 44.7 μmol) as a white solid. m/z (ESI-pos) M+1=564.4

Step I: 4N hydrogen chloride (1.5 mL in dioxane) was added to a vial containing tert-butyl (S)-17,26-difluoro-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (25 mg, 44.4 μmol) and the solution stirred at room temperature for 20 min. Upon completion, the solvent was evaporated to give (S)-17,26-difluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (22.2 mg, 44.4 μmol; assumed quant. yield) as a white solid which was used in the next step without further purification. m/z (ESI-pos) M+1=464.4

Step J: To a solution of (S)-17,26-difluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (22 mg, 44 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (23.5 mg, 57.2 μmol) in DMF (880 μL) was added N-ethyldiisopropylamine (38 μL, 220 μmol) and HATU (33.5 mg, 88 μmol). The mixture stirred at 35° C. for 2 h. The reaction solution was directly purified by Prep HPLC (Isco ACCQ Prep, Gemini column NX-C18, 250×21 mm, 20 mL/min, 45 minute separation, 20 to 80% H₂O/ACN with 0.1% Formic acid buffer), pure fractions were collected and lyophilized to give (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,26-difluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (21.4 mg, 24.9 μmol) as a white solid. m/z (ESI-pos) M+1=857.6; ¹H NMR (400 MHz, CHLOROFORM-D) δ 11.25 (d, J=16.1 Hz, 1H), 7.65-7.39 (m, 3H), 7.37-7.26 (m, 1H), 7.25-7.09 (m, 2H), 7.09-6.86 (m, 2H), 6.76-6.64 (m, 1H), 6.45-6.05 (m, 2H), 5.96-5.19 (m, 1H), 4.95-4.41 (m, 3H), 3.97-3.76 (m, 2H), 3.70-3.09 (m, 3H), 3.07-2.71 (m, 3H), 1.90 (t, J=5.2 Hz, 1H), 1.76 (td, J=17.5, 5.1 Hz, 1H), 1.67-1.60 (m, 2H), 1.59-1.50 (m, 9H), 1.33 (d, J=1.9 Hz, 3H), 1.30-1.22 (m, 3H), 1.21-1.01 (m, 3H).

### Example 147—Synthesis of Compound 147

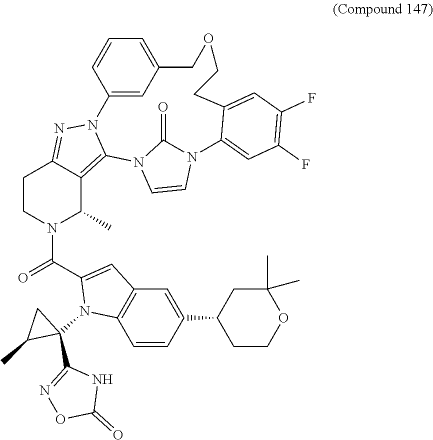

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,18-difluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 147 was synthesized following the procedure of Example 146 with 2-(bromomethyl)-1-fluoro-4-iodobenzene being replaced with m-(bromomethyl) iodobenzene and 2-(2-bromo-4-fluorophenyl) ethanol being replaced with 2-(2-bromo-4,5-difluorophenyl) ethanol in step A. m/z (ESI-pos) M+1=857.6; ¹H NMR (400 MHz, CHLOROFORM-D) δ 11.26 (d, J=21.2 Hz, 1H), 7.64-7.42 (m, 4H), 7.27-6.98 (m, 4H), 6.77-6.65 (m, 1H), 6.48-5.92 (m, 2H), 5.91-4.81 (m, 1H), 4.75-4.39 (m, 3H), 3.94-3.75 (m, 2H), 3.69-3.54 (m, 1H), 3.35 (dt, J=16.3, 5.8 Hz, 2H), 3.27-3.08 (m, 1H), 3.11-2.95 (m, 2H), 2.90-2.62 (m, 1H), 1.95-1.50 (m, 12H), 1.33 (d, J=5.4 Hz, 3H), 1.30-1.23 (m, 3H), 1.21-1.01 (m, 3H).

### Example 148—Synthesis of Compound 148

### Synthetic Scheme

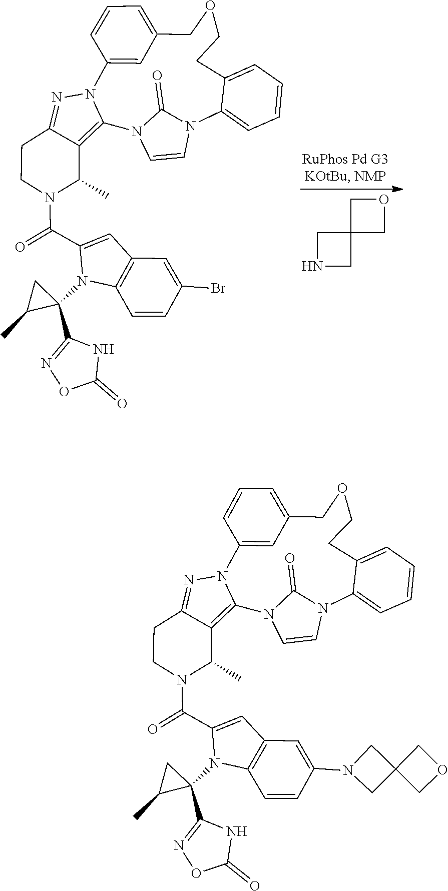 [SMILES: C[C@H]1c2c(nn3c2-n2ccn(c2=O)-c2ccccc2CCOCc2cccc-3c2)CCN1C(=O)c1cc2cc(Br)ccc2n1[C@@]1(c2noc(=O)[nH]2)C[C@@H]1C ; C1NCC12COC2 ; C[C@H]1c2c(nn3c2-n2ccn(c2=O)-c2ccccc2CCOCc2cccc-3c2)CCN1C(=O)c1cc2cc(N3CC4(COC4)C3)ccc2n1[C@@]1(c2noc(=O)[nH]2)C[C@@H]1C]

### Procedure for Synthesis of Compound 148

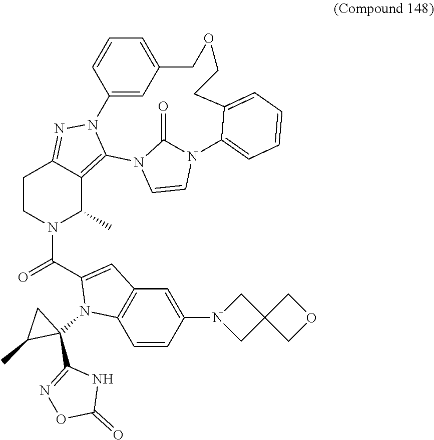

### (S)-8-methyl-7-({1-[(1S,2S)-2-methyl-1-(5-oxo-4,5-dihydro-1,2,4-oxadiazol-3-yl)cyclopropyl]-5-(2-oxa-6-aza-spiro[3.3]heptyl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: (S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (15 mg, 19 μmol), RuPhos Pd G3 (1.59 mg, 1.9 μmol), KOtBu (6.41 mg, 57.1 μmol), oxalic acid-2-oxa-6-azaspiro[3.3]heptane (1/2) (11 mg, 38.1 μmol) were dissolved in N-methyl-2-pyrrolidone (381 μL, 3.95 mmol) and degassed with Ar. The solution was heated to 100° C. overnight. Some starting material remained, so a second batch of all reagents was added, and the reaction was degassed and heated to 100° C. for an additional 2 h. The reaction mixture was cooled to room temperature, filtered, and purified by Prep HPLC (Isco ACCQ Prep, Gemini column NX-C18, 250×21 mm, 20 mL/min, 45 minute separation, 20 to 70% H₂O/ACN with 0.1% Formic acid buffer), pure fractions were collected and lyophilized to give (S)-8-methyl-7-({1-[(1S,2S)-2-methyl-1-(5-oxo-4,5-dihydro-1,2,4-oxadiazol-3-yl)cyclopropyl]-5-(2-oxa-6-aza-6-spiro[3.3]heptyl)-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (770 μg, 0.955 μmol). m/z (ESI-pos) M+1=806.6; ¹H NMR (400 MHz, CHLOROFORM-D) δ 11.31 (s, 1H), 7.60-7.27 (m, 7H), 7.24-7.13 (m, 2H), 6.66-6.57 (m, 3H), 6.52-5.89 (m, 2H), 5.78 (d, J=6.7 Hz, 1H), 4.84 (d, J=3.6 Hz, 4H), 4.69-4.44 (m, 3H), 4.02 (s, 4H), 3.61 (d, J=14.1 Hz, 1H), 3.46-2.29 (m, 4H), 1.95-1.74 (m, 1H), 1.62 (d, J=6.7 Hz, 3H), 1.48 (d, J=9.3 Hz, 1H), 1.38-0.78 (m, 6H).

### Example 149—Synthesis of Compound 149

### Synthetic Scheme

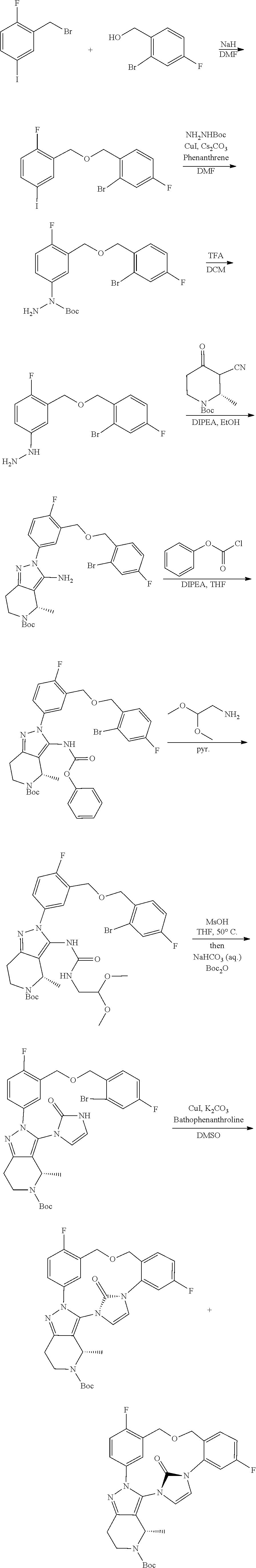

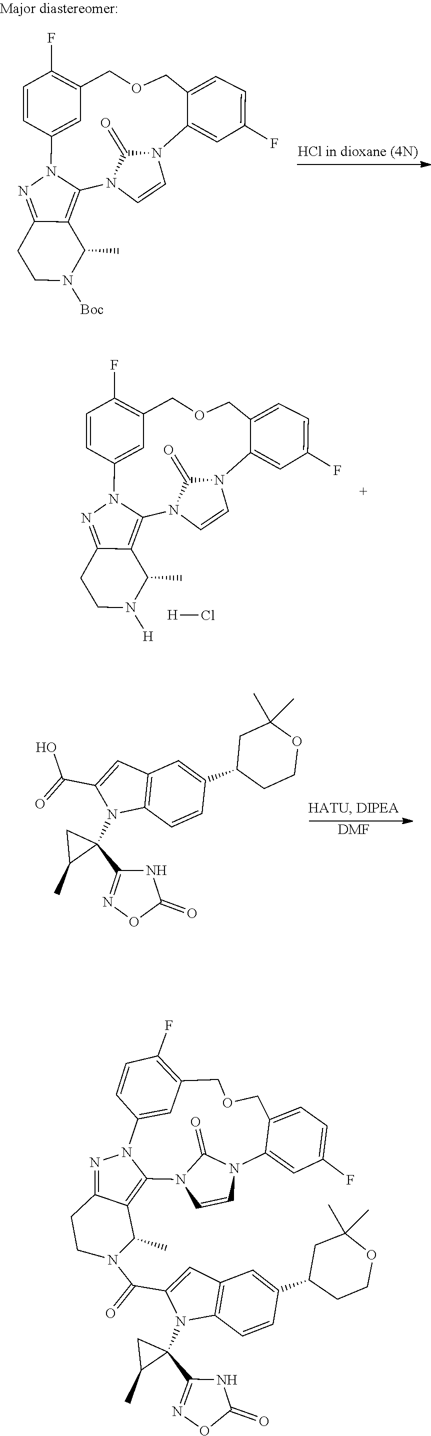

### Procedure for Synthesis of Compound 149

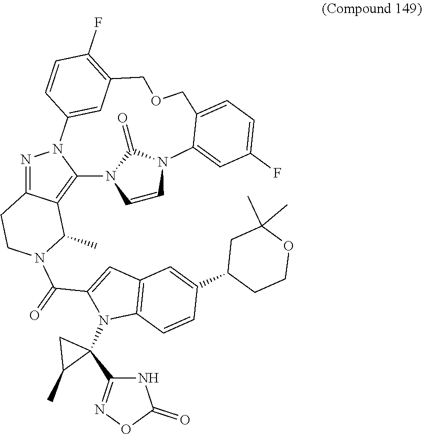

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (2-bromo-4-fluorophenyl)methanol (0.6 g, 2.93 mmol) and 2-(bromomethyl)-1-fluoro-4-iodobenzene (1.01 g, 3.22 mmol) in DMF (5.85 mL) was added sodium hydride 60% w/w (123 mg, 3.07 mmol) at 0° C. The reaction was stirred at 0° C. for 30 min and at room temperature for 30 min. The reaction mixture was quenched with sat. aq. NH₄Cl, diluted with ethyl acetate, washed with brine. The organic layer was dried over Na₂SO₄ and concentrated in vacuo. The crude product was purified by column chromatography (0-10% EtOAc in heptane) to give 2-{[(2-bromo-4-fluorophenyl)methoxy]methyl}-1-fluoro-4-iodobenzene (1.13 g, 2.57 mmol) as a clear oil. m/z (ESI-pos) M+1=not found—does not ionize

Step B: A solution of 1-bromo-4-fluoro-2-{[(2-fluoro-5-iodophenyl)methoxy]methyl}benzene (1.13 g, 2.57 mmol), tert-butyl carbazate (442 mg, 3.34 mmol), dicaesium carbonate (1.17 g, 3.6 mmol), copper iodide (24.5 mg, 129 μmol), and 4,5-diazaphenanthrene (92.7 mg, 514 μmol) in DMF (2.57 mL) was sparged with Ar for 5 min and heated to 80° C. for 2.5 h. The solution was taken up in EtOAc (20 mL) and washed with brine (3×20 mL). The organics were dried over Na₂SO₄, filtered, and concentrated. The crude product purified by silica gel chromatography (5-30% EtOAc in heptane) to give tert-butyl 2-(3-{[(2-bromo-4-fluorophenyl)methoxy]methyl}-4-fluorophenyl)carbazate (862 mg, 1.94 mmol) as a yellow oil. m/z (ESI-pos) M+1-tBu=387.1/389.1

Step C: To a solution of tert-butyl 2-(3-{[(2-bromo-4-fluorophenyl)methoxy]methyl}-4-fluorophenyl)carbazate (862 mg, 1.94 mmol) in DCM (6.5 mL), trifluoroacetic acid (1.49 mL, 19.4 mmol) was added. The reaction was stirred at room temperature for 3 h. Upon completion, the reaction was *carefully* quenched with sat. aq. Na₂CO₃ (~30 mL), extracted DCM (1×50 mL), then washed with water (2×50 mL) and brine (1×50 mL). The combined aqueous phases were extracted with 20% IPA/CHCl₃ (2×50 mL) and the combined organics were washed with sat. aq. NaHCO₃ (1×50 mL) and then dried over Na₂SO₄, filtered, and concentrated in vacuo to provide (3-{[(2-bromo-4-fluorophenyl)methoxy]methyl}-4-fluorophenyl)hydrazine (653 mg, 1.9 mmol) as a yellow oil which was used without purification in the following step. m/z (ESI-pos) M+1=343.1/345.1

Step D: To a solution of crude (3-{[(2-bromo-4-fluorophenyl)methoxy]methyl}-4-fluorophenyl)hydrazine (653 mg, 1.9 mmol) in EtOH (7.6 mL) was added N-ethyldiisopropylamine (331 μL, 1.9 mmol) and tert-butyl(2S)-3-cyano-2-methyl-4-oxo-1-piperidinecarboxylate (544 mg, 2.28 mmol). The mixture was stirred at 80° C. for 20 h. After completion, the reaction mixture was cooled to ambient temperature and the volatiles were evaporated under reduced pressure. The crude compound was purified by column chromatography (15-60% EtOAc in heptane) to give tert-butyl (S)-3-amino-2-(3-{[(2-bromo-4-fluorophenyl)methoxy]methyl}-4-fluorophenyl)-4-methyl-1,2,5-triaza-2,4,6,7-tetrahydro-5H-indene-5-carboxylate (614 mg, 1.09 mmol) as a yellow solid over 2 steps. m/z (ESI-pos) M+1=563.3/565.3

Step E: To a solution of tert-butyl (S)-3-amino-2-(3-{[(2-bromo-4-fluorophenyl)methoxy]methyl}-4-fluorophenyl)-4-methyl-1,2,5-triaza-2,4,6,7-tetrahydro-5H-indene-5-carboxylate (614 mg, 1.09 mmol) in THF (5.45 mL) was added N-ethyldiisopropylamine (569 μL, 3.27 mmol) followed by benzenyl chloroformate (273 μL, 2.18 mmol). The reaction mixture was stirred at 25° C. for 30 min. The reaction mixture was then directly concentrated under reduced pressure to give crude tert-butyl (S)-2-(3-{[(2-bromo-4-fluorophenyl)methoxy]methyl}-4-fluorophenyl)-4-methyl-3-[phenyl(oxycarbonylamino)]-1,2,5-triaza-2,4,6,7-tetrahydro-5H-indene-5-carboxylate (745 mg, 1.09 mmol) as a yellow oil, which was used directly in the next step without any further purification. m/z (ESI-pos) M+1=683.3/685.3

Step F: Crude tert-butyl (S)-2-(3-{[(2-bromo-4-fluorophenyl)methoxy]methyl}-4-fluorophenyl)-4-methyl-3-[phenyl(oxycarbonylamino)]-1,2,5-triaza-2,4,6,7-tetrahydro-5H-indene-5-carboxylate (745 mg, 1.09 mmol) was dissolved in pyridine (3.63 mL) and then 2,2-dimethoxyethylamine (178 μL, 1.63 mmol) was added. The mixture was stirred at room temperature for 30 min and then directly concentrated under reduced pressure. The crude yellow residue was purified by column chromatography (5-80% EtOAc in heptane) to give tert-butyl (S)-2-(3-{[(2-bromo-4-fluorophenyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (430 mg, 619 μmol) as a white solid over 2 steps. m/z (ESI-pos) M+1=694.4/696.3

Step G: To a solution of tert-butyl (S)-2-(3-{[(2-bromo-4-fluorophenyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (430 mg, 619 μmol) in THF (3.1 mL) was added methanesulfonic acid (121 μL, 1.86 mmol). The mixture was stirred at 55° C. for 1 h and cooled to ambient temperature. Saturated aqueous NaHCO₃ was added until pH 8 then tert-butyl-tert-butyl(oxycarbonyloxy) formylate (149 mg, 681 μmol) was added, and the resulting mixture was stirred at room temperature for 30 min. The reaction mixture was diluted with water (20 mL) and was extracted with ethyl acetate (2×30 mL). The combined organic layers were washed with brine (30 mL) and dried over anhydrous Na₂SO₄, filtered and concentrated under reduced pressure. The crude compound was purified by column chromatography (5-90% EtOAc in heptane) to give tert-butyl (S)-2-(3-{[(2-bromo-4-fluorophenyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (185 mg, 293 μmol) as an off-white solid. m/z (ESI-pos) M+1=630.3/632.3

Step H: A solution of tert-butyl (S)-2-(3-{[(2-bromo-4-fluorophenyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (185 mg, 294 μmol), copper iodide (42 mg, 220 μmol), Bathophenanthroline (97.7 mg, 294 μmol), and dipotassium carbonate (102 mg, 735 μmol) in DMSO (9.8 mL) was sparged with Ar for 5 minutes, and then heated to 130° C. overnight in a sealed vial for 2 d. Upon cooling to ambient temperature, the reaction was filtered through a pad of Celite (rinsing with EtOAc) then washed with water and sat. aq. NH₄Cl. The workup solution (organic and aqueous) was again filtered through a pad of celite. The filtrate was partitioned and the organic phase was washed with brine, dried with sodium sulfate, filtered, concentrated. The crude was purified by silica gel chromatography (5-50% EtOAc in heptane) to give two diastereomers, whose structure was arbitrarily assigned, tert-butyl (8S,10M)-17,25-difluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (43.4 mg, 79 μmol) and tert-butyl (8S,10P)-17,25-difluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (12.8 mg, 23.3 μmol). m/z (ESI-pos) M+1=550.4

Step I: Hydrogen chloride (1.5 mL; 4N in dioxane) was added to a vial containing tert-butyl (8S,10M)-17,25-difluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (15 mg, 27.3 μmol) and the solution stirred at room temperature for 20 min. Upon completion, the solvent was evaporated to give the crude (8S,10M)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (13.3 mg, 27.4 μmol) as a white solid which was used in the next step without purification. m/z (ESI-pos) M+1=450.4

Step J: To a solution of (8S,10M)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (13.3 mg, 27.4 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (14.6 mg, 35.6 μmol) in DMF (550 μL) was added N-ethyldiisopropylamine (23.8 μL, 137 μmol) and HATU (20.8 mg, 54.7 μmol). The mixture stirred at room temperature overnight. The reaction was directly purified by Prep HPLC (Isco ACCQ Prep, Gemini column NX-C18, 250×21 mm, 20 ml/min, 45 minute separation, 20 to 80% H₂O/ACN with 0.1% Formic acid buffer), pure fractions were collected and lyophilized to give (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (19.2 mg, 22.8 μmol). m/z (ESI-pos) M+1=843.6; ¹H NMR (400 MHz, CHLOROFORM-D) δ 11.30 (s, 1H), 7.65-7.48 (m, 4H), 7.33-7.27 (m, 2H), 7.19 (td, J=8.5, 3.4 Hz, 3H), 6.74 (d, J=35.0 Hz, 1H), 6.48-5.86 (m, 2H), 5.85-5.21 (m, 1H), 4.99-4.41 (m, 3H), 4.32-4.08 (m, 2H), 3.91-3.77 (m, 2H), 3.70-3.34 (m, 1H), 3.24-2.93 (m, 3H), 2.00-1.58 (m, 8H), 1.55-1.50 (m, 2H), 1.33 (s, 3H), 1.27 (s, 3H), 1.16 (dd, J=30.2, 6.0 Hz, 3H).

### Example 150—Synthesis of Compound 150

### Synthetic Scheme

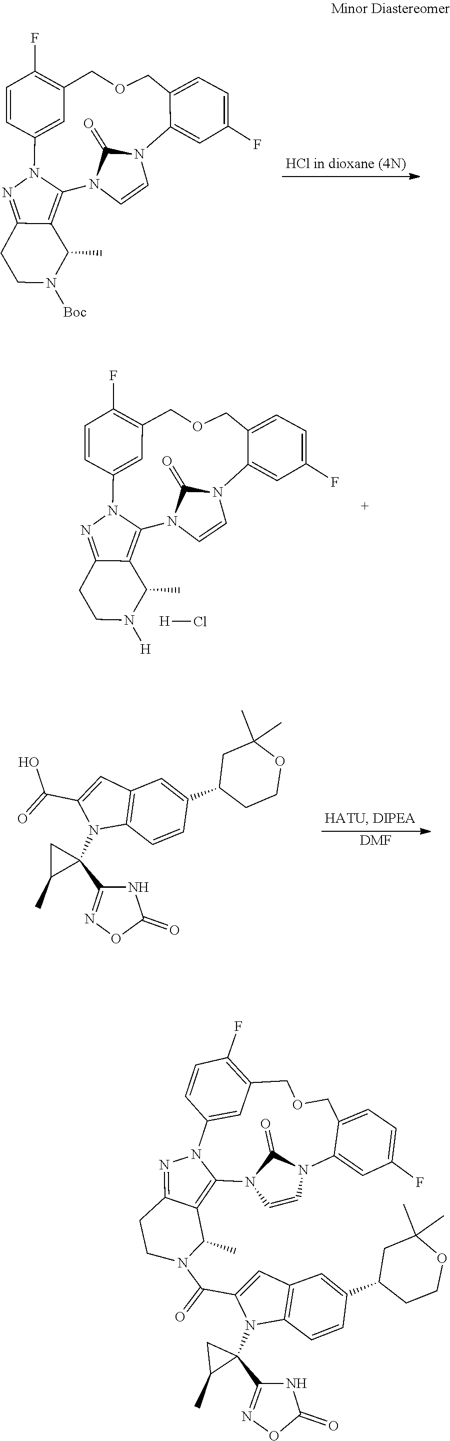

### Procedure for Synthesis of Compound 150

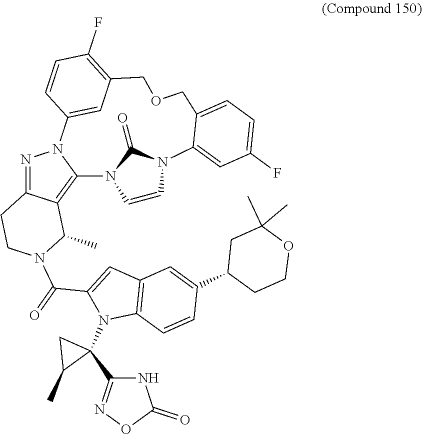

### (8S,10P)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: Hydrogen chloride (1 mL; 4N in dioxane) was added to a vial containing tert-butyl (8S,10P)-17,25-difluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (12.8 mg, 23.3 μmol) and the solution stirred at room temperature for 20 min. Upon completion, the solvent was evaporated to give the crude residue, (8S,10P)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (11.3 mg, 23.3 μmol), that was used in the next step without purification. m/z (ESI-pos) M+1=450.4

Step B: To a solution of (8S,10P)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (11.3 mg, 23.3 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (12.4 mg, 30.2 μmol) in DMF (500 μL) was added N-ethyldiisopropylamine (20.3 μL, 116 μmol) and HATU (17.7 mg, 46.5 μmol). The mixture stirred at room temperature overnight. The reaction mixture was then directly purified by Prep HPLC (Isco ACCQ Prep, Gemini column NX-C18, 250×21 mm, 20 ml/min, 45 minute separation, 20 to 80% H₂O/ACN with 0.1% Formic acid buffer), pure fractions were collected and lyophilized to give (8S,10P)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (12.4 mg, 14.8 μmol). m/z (ESI-pos) M+1=843.6; ¹H NMR (400 MHz, CHLOROFORM-D) δ 11.23 (s, 1H), 7.65-7.41 (m, 4H), 7.36-7.26 (m, 1H), 7.24-7.08 (m, 3H), 6.70 (d, J=1.3 Hz, 1H), 6.38 (dd, J=17.6, 3.1 Hz, 1H), 6.19 (dd, J=24.2, 3.1 Hz, 1H), 5.55 (dq, J=243.8, 6.6 Hz, 1H), 4.89-4.43 (m, 3H), 4.29-3.98 (m, 2H), 3.91-3.79 (m, 2H), 3.64-3.32 (m, 1H), 3.24-2.94 (m, 3H), 1.96-1.37 (m, 9H), 1.30 (dd, J=24.8, 2.6 Hz, 7H), 1.09 (dd, J=85.2, 5.9 Hz, 4H).

### Example 151—Synthesis of Compound 151

### Synthetic Scheme

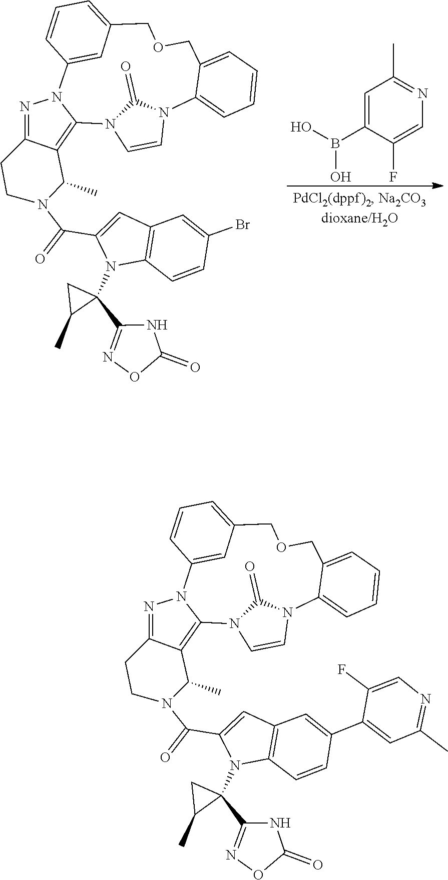

### Procedure for Synthesis of Compound 151

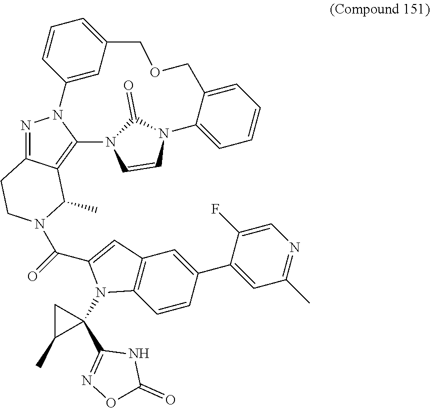

### (8S,10M)-7-{[5-(5-fluoro-2-methyl-4-pyridyl)-1-[(1S,2S)-2-methyl-1-(5-oxo-4,5-dihydro-1,2,4-oxadiazol-3-yl)cyclopropyl]-2-indolyl]carbonyl}-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: A solution of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (30 mg, 38.8 μmol), PdCl₂(dppf) (3.17 mg, 3.88 μmol), (5-fluoro-2-methyl-4-pyridyl)boranediol (18 mg, 116 μmol) and disodium carbonate (20.5 mg, 194 μmol) in 1,4-dioxane (1 mL) and water (390 μL) was heated to 80° C. for 1 h. The solution was partitioned between EtOAc and water, the organic layer was dried with Na₂SO₄, filtered and concentrated. The crude product was purified by reverse-phase chromatography (25-90% MeCN/H₂O with 0.1% FA) and the pure fractions were lyophilized to afford (8S,10M)-7-{[5-(5-fluoro-2-methyl-4-pyridyl)-1-[(1S,2S)-2-methyl-1-(5-oxo-4,5-dihydro-1,2,4-oxadiazol-3-yl)cyclopropyl]-2-indolyl]carbonyl}-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (17.9 mg, 22.3 μmol) as an off-white solid. m/z (ESI-pos) M+1=804.5; ¹H NMR (400 MHz, CHLOROFORM-D) δ 11.30 (s, 1H), 8.41 (d, J=2.4 Hz, 1H), 8.10-7.89 (m, 1H), 7.78-7.40 (m, 8H), 7.36-7.26 (m, 1H), 7.19 (dd, J=11.6, 5.2 Hz, 1H), 6.85 (d, J=30.8 Hz, 1H), 6.48-5.87 (m, 2H), 5.87-5.23 (m, 1H), 4.98-4.06 (m, 4H), 4.67 (s, 2H), 3.74-3.38 (m, 1H), 3.25-2.84 (m, 2H), 2.59 (s, 3H), 2.01-1.78 (m, 2H), 1.68 (d, J=6.7 Hz, 3H), 1.66-1.51 (m, 1H), 1.46-1.27 (m, 1H), 1.19 (dd, J=29.1, 6.1 Hz, 3H).

### Example 152 and 153—Synthesis of Compound 152 and 153

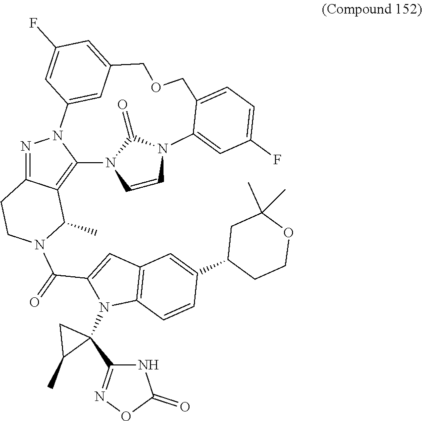

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,26-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compounds 152 and 153 were synthesized following the procedure of Example 149 and 150, respectfully, with 2-(bromomethyl)-1-fluoro-4-iodobenzene being replaced with 1-(bromomethyl)-3-fluoro-5-iodobenzene in step A. Major diastereomer: m/z (ESI-pos) M+1=843.6; ¹H NMR (400 MHz, CHLOROFORM-D) δ 11.28 (s, 1H), 7.66-7.26 (m, 4H), 7.21-7.05 (m, 2H), 6.97-6.87 (m, 2H), 6.80-6.67 (m, 1H), 6.55-5.90 (m, 2H), 5.86-5.22 (m, 1H), 4.96-4.41 (m, 3H), 4.29 (dd, J=32.2, 9.5 Hz, 1H), 4.05 (d, J=9.5 Hz, 1H), 3.95-3.77 (m, 2H), 3.62 (td, J=13.1, 4.1 Hz, 1H), 3.20-2.93 (m, 3H), 1.88 (dt, J=23.9, 6.0 Hz, 1H), 1.82-1.70 (m, 3H), 1.69-1.62 (m, 3H), 1.60-1.48 (m, 4H), 1.33 (s, 3H), 1.27 (s, 3H), 1.16 (dd, J=28.6, 6.0 Hz, 3H).

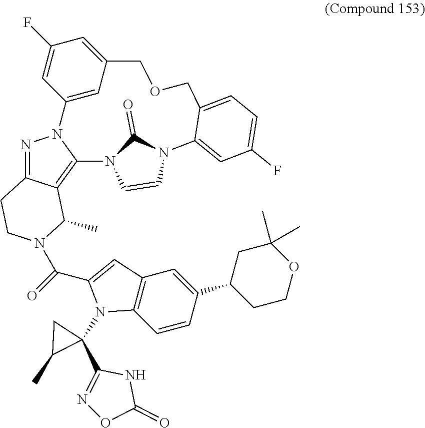

### (8S,10P)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,26-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Minor diastereomer: m/z (ESI-pos) M+1=843.6; ¹H NMR (400 MHz, CHLOROFORM-D) δ 11.20 (s, 1H), 7.61-7.36 (m, 5H), 7.24-7.05 (m, 2H), 7.04-6.65 (m, 3H), 6.52-6.21 (m, 2H), 5.55 (dq, J=241.5, 6.6 Hz, 1H), 4.94-4.57 (m, 2H), 4.55-4.19 (m, 2H), 3.99 (dd, J=48.5, 9.5 Hz, 1H), 3.90-3.77 (m, 2H), 3.48 (dtd, J=77.1, 13.0, 4.2 Hz, 1H), 3.24-2.95 (m, 3H), 1.94-1.69 (m, 5H), 1.67-1.58 (m, 3H), 1.54-1.35 (m, 1H), 1.33 (s, 3H), 1.27 (s, 3H), 1.25 (d, J=3.3 Hz, 1H), 1.09 (dd, J=82.9, 5.9 Hz, 3H).

### Example 154—Synthesis of Compound 154

### Synthetic Scheme

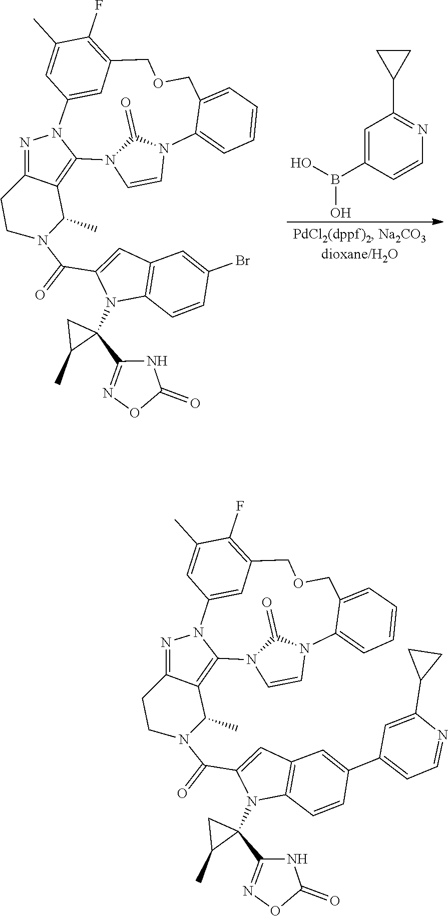

### Procedure for Synthesis of Compound 154

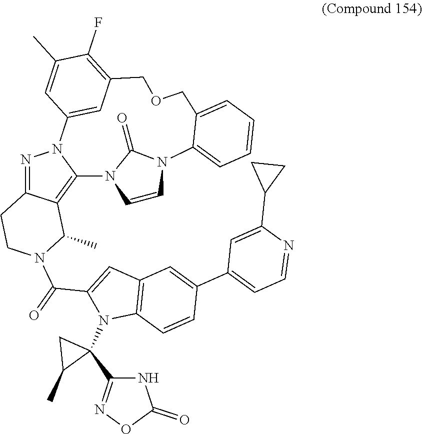

### (8S,10M)-7-{[5-(2-cyclopropyl-4-pyridyl)-1-[(1S,2S)-2-methyl-1-(5-oxo-4,5-dihydro-1,2,4-oxadiazol-3-yl)cyclopropyl]-2-indolyl]carbonyl}-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: A solution of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (15 mg, 18.6 μmol) PdCl₂(dppf) (1.52 mg, 1.86 μmol), (2-cyclopropyl-4-pyridyl)boranediol (9.1 mg, 55.9 μmol) and disodium carbonate (9.87 mg, 93.1 μmol) in 1,4-dioxane (465 μL) and water (186 μL) was heated to 80° C. for 1 hour. The solution was partitioned between EtOAc and water, the organic layer was dried with Na₂SO₄, filtered and concentrated. The crude residue was purified by reverse-phase chromatography (25-60% MeCN/H₂O with 0.1% FA) and the pure fractions were lyophilized to afford (8S,10M)-7-{[5-(2-cyclopropyl-4-pyridyl)-1-[(1S,2S)-2-methyl-1-(5-oxo-4,5-dihydro-1,2,4-oxadiazol-3-yl)cyclopropyl]-2-indolyl]carbonyl}-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (12.5 mg, 14.8 μmol) as an off-white solid. m/z (ESI-pos) M+1=844.5; ¹H NMR (400 MHz, CHLOROFORM-D) δ 11.29 (s, 1H), 8.48 (d, J=5.2 Hz, 1H), 7.98-7.91 (m, 1H), 7.74 (d, J=8.6 Hz, 1H), 7.64 (dd, J=8.6, 1.8 Hz, 1H), 7.58-7.32 (m, 6H), 7.28 (dd, J=5.2, 1.8 Hz, 1H), 7.14-7.00 (m, 1H), 6.92-6.75 (m, 1H), 6.53-5.86 (m, 2H), 5.86-5.22 (m, 1H), 4.97-4.12 (m, 5H), 3.67 (td, J=13.0, 4.0 Hz, 1H), 3.26-2.98 (m, 2H), 2.35-2.30 (m, 3H), 2.10 (ddd, J=13.1, 8.1, 4.9 Hz, 1H), 1.94 (dt, J=24.5, 6.3 Hz, 1H), 1.75 (dd, J=56.3, 6.7 Hz, 3H), 1.57 (s, 2H), 1.19 (dd, J=29.5, 6.1 Hz, 3H), 1.12-0.99 (m, 4H).

### Example 155—Synthesis of Compound 155

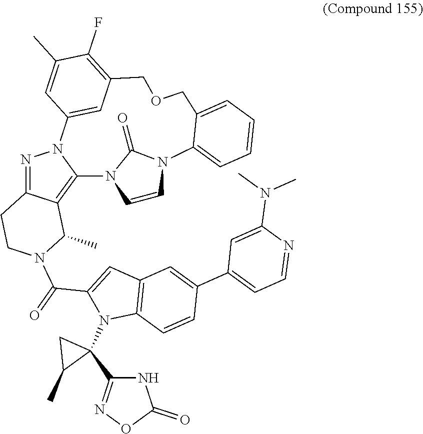

### (8S,10M)-7-({5-[2-(dimethylamino)-4-pyridyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 155 was synthesized following the procedure of Example 154 with (2-cyclopropyl-4-pyridyl)boranediol being replaced with N,N-dimethyl[4-(dihydroxyboryl)-2-pyridyl]amine in step A. m/z (ESI-pos) M+1=847.5; ¹H NMR (400 MHz, CHLOROFORM-D) δ 11.28 (s, 1H), 8.25 (d, J=5.4 Hz, 1H), 7.93 (d, J=13.2 Hz, 1H), 7.74 (d, J=8.7 Hz, 1H), 7.63 (d, J=8.6 Hz, 1H), 7.58-7.42 (m, 5H), 7.11 (s, 1H), 6.94-6.69 (m, 3H), 6.51-5.87 (m, 2H), 5.85-5.19 (m, 1H), 4.74 (q, J=14.6 Hz, 2H), 4.48 (d, J=11.7 Hz, 1H), 4.31 (d, J=9.3 Hz, 1H), 4.16 (d, J=9.2 Hz, 1H), 3.66 (t, J=11.2 Hz, 1H), 3.20 (s, 6H), 3.08 (t, J=19.2 Hz, 2H), 2.32 (d, J=9.4 Hz, 3H), 1.96 (t, J=6.1 Hz, 1H), 1.68 (d, J=6.7 Hz, 3H), 1.58-1.36 (m, 2H), 1.19 (dd, J=29.7, 6.1 Hz, 3H).

### Example 156—Synthesis of Compound 156

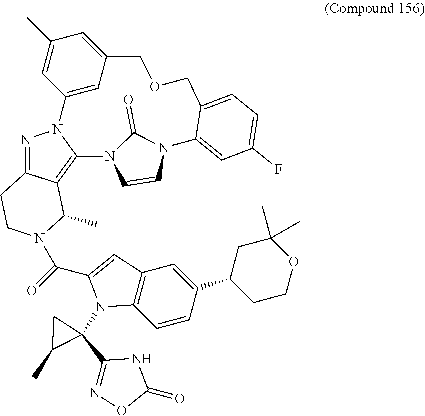

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 156 was synthesized following the procedure of Example 149, with 2-(bromomethyl)-1-fluoro-4-iodobenzene and (2-bromo-4-fluorophenyl)methanol being replaced 2-bromo-1-(bromomethyl)-4-fluorobenzene and (3-iodo-5-tolyl)methanol in step A. m/z (ESI-pos) M+1=839.6; ¹H NMR (400 MHz, CHLOROFORM-D) δ 11.31 (s, 1H), 7.66-7.26 (m, 5H), 7.20-7.05 (m, 2H), 7.00 (d, J=10.4 Hz, 2H), 6.74 (d, J=31.2 Hz, 1H), 6.49-5.86 (m, 2H), 5.85-5.22 (m, 1H), 4.96-4.57 (m, 2H), 4.48 (dt, J=13.5, 6.7 Hz, 1H), 4.21 (dd, J=28.1, 9.5 Hz, 1H), 4.06 (t, J=9.0 Hz, 1H), 3.91-3.77 (m, 2H), 3.71-3.33 (m, 1H), 3.24-2.94 (m, 4H), 2.40 (d, J=9.5 Hz, 3H), 1.88 (dt, J=25.8, 6.1 Hz, 1H), 1.81-1.70 (m, 2H), 1.65 (d, J=6.8 Hz, 3H), 1.61-1.49 (m, 2H), 1.33 (s, 3H), 1.26 (d, J=13.4 Hz, 4H), 1.16 (dd, J=30.7, 6.0 Hz, 3H).

### Example 157—Synthesis of Compound 157

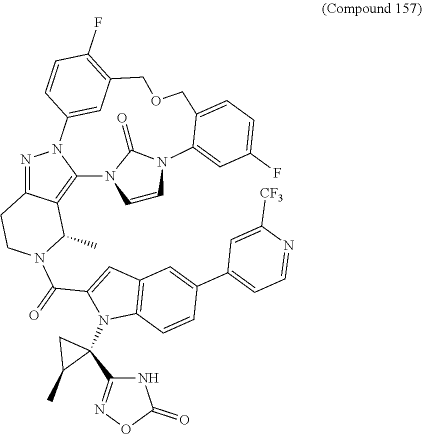

### (8S,10M)-17,25-difluoro-8-methyl-7-({1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 157 was synthesized following the procedure of Examples 149 and 151, with (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one and (5-fluoro-2-methyl-4-pyridyl)boranediol being replaced with (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one and [2-(trifluoromethyl)-4-pyridyl]boranediol in step A. m/z (ESI-pos) M+1=876.4; ¹H NMR (400 MHz, CHLOROFORM-D) δ 11.20 (s, 1H), 8.77 (d, J=5.1 Hz, 1H), 8.05-7.97 (m, 1H), 7.93 (d, J=1.6 Hz, 1H), 7.82-7.43 (m, 5H), 7.29 (dd, J=6.1, 2.8 Hz, 1H), 7.22-7.04 (m, 3H), 6.88 (d, J=36.6 Hz, 1H), 6.47-5.88 (m, 2H), 5.87-5.21 (m, 1H), 4.99-4.38 (m, 3H), 4.31-4.08 (m, 2H), 3.78-3.37 (m, 1H), 3.13 (dtd, J=31.6, 16.0, 4.5 Hz, 2H), 2.12-1.89 (m, 1H), 1.76 (dd, J=57.3, 6.7 Hz, 3H), 1.63-1.54 (m, 1H), 1.33-1.11 (m, 4H).

### Example 158—Synthesis of Compound 158

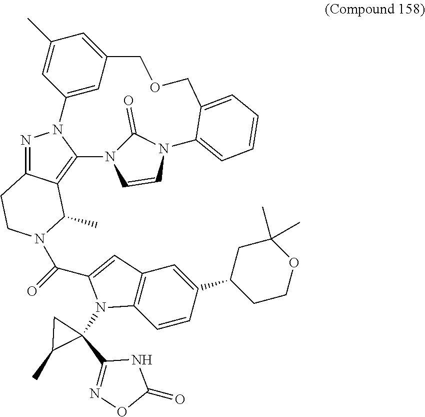

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 158 was synthesized following the procedure of Example 69, with 2-(bromomethyl)-1-fluoro-4-iodobenzene and (o-bromophenyl)methanol being replaced with o-bromo (bromomethyl)benzene and (3-iodo-5-tolyl)methanol in step A. m/z (ESI-pos) M+1=821.6; ¹H NMR (400 MHz, CHLOROFORM-D) δ 11.30 (d, J=21.3 Hz, 1H), 7.65-7.26 (m, 8H), 7.08-6.93 (m, 2H), 6.84-6.69 (m, 1H), 6.50-5.87 (m, 2H), 5.84-5.21 (m, 1H), 4.98-4.41 (m, 3H), 4.38-4.01 (m, 2H), 3.92-3.77 (m, 2H), 3.63 (td, J=13.1, 4.0 Hz, 1H), 3.25-2.94 (m, 3H), 2.40 (d, J=9.5 Hz, 3H), 1.99 (s, 1H), 1.88 (dt, J=24.9, 6.1 Hz, 1H), 1.82-1.48 (m, 7H), 1.31 (d, J=23.6 Hz, 7H), 1.16 (dd, J=29.6, 6.0 Hz, 3H).

### Example 159—Synthesis of Compound 159

### Synthetic Scheme

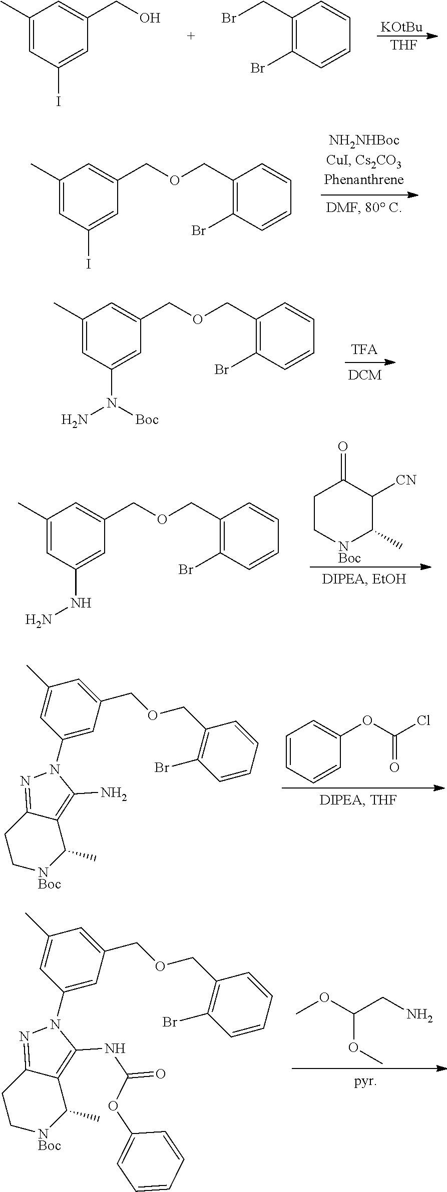

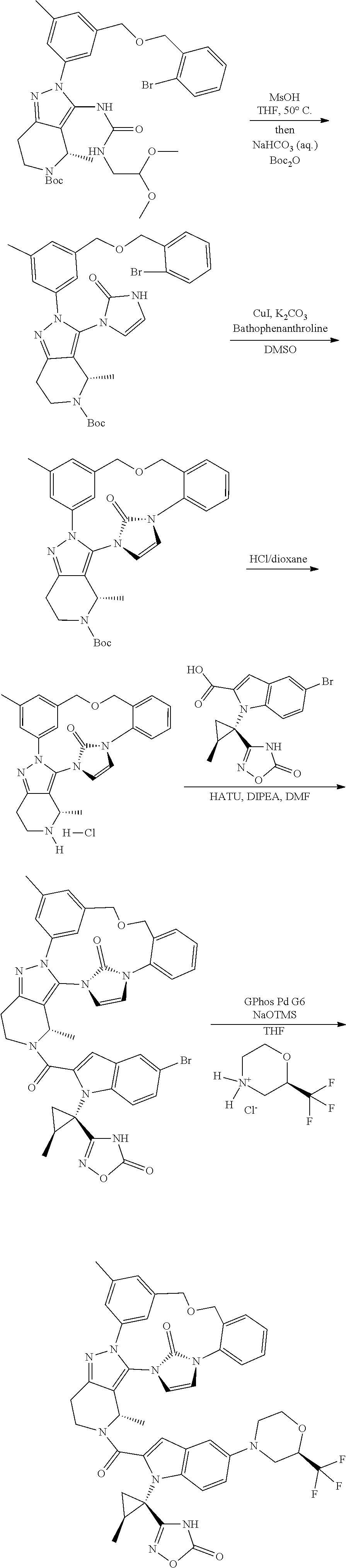

### Procedure for Synthesis of Compound 159

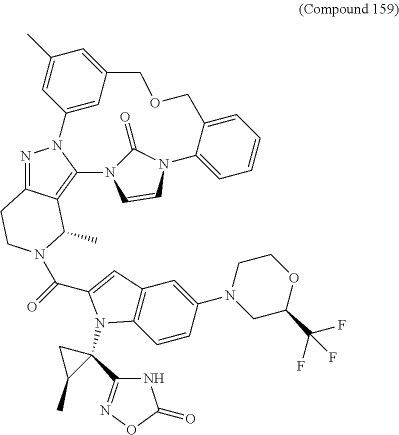

### (8S,10M)-8,26-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Step A: To a solution of (3-iodo-5-tolyl)methanol (998 mg, 4.02 mmol) and o-bromo (bromomethyl)benzene (1.01 g, 4.02 mmol) in DMF (8 mL) was added sodium hydride 60% w/w (169 mg, 4.22 mmol) at 0° C. The reaction was stirred at 0° C. for 30 min and then at room temperature for an additional 30 min. The reaction mixture was quenched with sat. aq. NH₄Cl (20 mL), diluted with ethyl acetate (50 mL), and washed with brine (2×50 mL). The organic layer was dried over Na₂SO₄ and concentrated in vacuo to get crude The crude product was purified by column chromatography (0-10% EtOAc in heptane) to give 5-{[(o-bromophenyl)methoxy]methyl}-3-iodotoluene (1.49 g, 3.57 mmol) as a colorless oil. m/z (ESI-pos) M+1=not found-does not ionize

Step B: A solution of 5-{[(o-bromophenyl)methoxy]methyl}-3-iodotoluene (1.49 g, 3.57 mmol), tert-butyl carbazate (614 mg, 4.64 mmol), dicaesium carbonate (1.63 g, 5 mmol), copper iodide (34 mg, 179 μmol), and 4,5-diazaphenanthrene (129 mg, 714 μmol) in DMF (3.6 mL) was sparged with Ar for 5 min and heated to 80° C. for 2.5 h. The solution was taken up in EtOAc (20 mL), filtered through Celite, and washed with brine (3×20 mL). The organics were dried over Na₂SO₄, filtered, and concentrated. The crude product purified by silica gel chromatography (5-30% EtOAc in heptane) to give tert-butyl 2-(m-{[2-(2-bromo-4,5-difluorophenyl)ethoxy]methyl}phenyl)carbazate (347 mg, 759 μmol) as a colorless oil. m/z (ESI-pos) M+1=365.1/367.2

Step C: To a solution of tert-butyl 2-(3-{[(o-bromophenyl)methoxy]methyl}-5-tolyl)carbazate (1.24 g, 2.94 mmol) in DCM (9.8 mL) and trifluoroacetic acid (3.38 mL, 44.1 mmol) was added at room temperature. The reaction was stirred for 2.5 h. Upon completion, the reaction was *carefully* quenched with sat. aq. Na₂CO₃ (~30 mL), extracted with DCM (2×20 mL), then washed with NaHCO₃ (2×20 mL) and brine (1×20 mL), and then dried over Na₂SO₄, filtered, and concentrated in vacuo to provide (3-{[(o-bromophenyl)methoxy]methyl}-5-tolyl) hydrazine (866 mg, 2.7 mmol) as a yellow oil which was used without purification in the following step. m/z (ESI-pos) M+1=321.1/323.1

Step D: To a solution of (3-{[(o-bromophenyl)methoxy]methyl}-5-tolyl) hydrazine (866 mg, 2.7 mmol) in EtOH (10.8 mL) was added N-ethyldiisopropylamine (470 μL, 2.7 mmol) then tert-butyl(2S)-3-cyano-2-methyl-4-oxo-1-piperidinecarboxylate (771 mg, 3.24 mmol) at room temperature. The mixture was stirred at 80° C. for 24 h. After completion, the reaction mixture was cooled to ambient temperature and the volatiles evaporated under reduced pressure. The crude compound was purified by column chromatography (10-60% EtOAc in heptane) to tert-butyl (S)-3-amino-2-(3-{[(o-bromophenyl)methoxy]methyl}-5-tolyl)-4-methyl-1,2,5-triaza-2,4,6,7-tetrahydro-5H-indene-5-carboxylate (835 mg, 1.54 mmol) as a yellow solid over 2 steps. m/z (ESI-pos) M+1=541.3/543.3

Step E: To a solution of tert-butyl (S)-3-amino-2-(3-{[(o-bromophenyl)methoxy]methyl}-5-tolyl)-4-methyl-1,2,5-triaza-2,4,6,7-tetrahydro-5H-indene-5-carboxylate (835 mg, 1.54 mmol) in THF (7.7 mL) was added N-ethyldiisopropylamine (806 μL, 4.63 mmol) followed by benzenyl chloroformate (387 μL, 3.08 mmol). The reaction was stirred at 25° C. for 30 min. The reaction mixture was directly concentrated under reduced pressure to give the crude tert-butyl (S)-2-(3-{[(o-bromophenyl)methoxy]methyl}-5-tolyl)-4-methyl-3-[phenyl(oxycarbonylamino)]-1,2,5-triaza-2,4,6,7-tetrahydro-5H-indene-5-carboxylate (1.02 g, 1.54 mmol, assume quant. yield.) as a yellow solid, which was used directly in the next step without any further purification. m/z (ESI-pos) M+1=661.4/663.4

Step F: Crude tert-butyl (S)-2-(3-{[(o-bromophenyl)methoxy]methyl}-5-tolyl)-4-methyl-3-[phenyl(oxycarbonylamino)]-1,2,5-triaza-2,4,6,7-tetrahydro-5H-indene-5-carboxylate (1.02 g, 1.54 mmol) was dissolved in pyridine (5.2 mL) and then 2,2-dimethoxyethylamine (252 μL, 2.31 mmol) was added. The mixture was stirred at room temperature for 30 min and then directly concentrated under reduced pressure. The crude residue was purified by column chromatography (5-70% EtOAc in heptane) to give tert-butyl (S)-2-(3-{[(o-bromophenyl)methoxy]methyl}-5-tolyl)-3-[3-(2,2-dimethoxyethyl)ureido]-4-methyl-1,2,5-triaza-2,4,6,7-tetrahydro-5H-indene-5-carboxylate (670 mg, 996 μmol) as a white solid over 2 steps. m/z (ESI-pos) M+1=672.4/674.4

Step G: To a solution of tert-butyl (S)-2-(3-{[(o-bromophenyl)methoxy]methyl}-5-tolyl)-3-[3-(2,2-dimethoxyethyl)ureido]-4-methyl-1,2,5-triaza-2,4,6,7-tetrahydro-5H-indene-5-carboxylate (670 mg, 996 μmol) in THF (5 mL) was added methanesulfonic acid (194 μL, 2.99 mmol). The mixture was stirred at 55° C. for 1 h and then cooled to ambient temperature. Saturated aq. NaHCO₃ was added until pH 8 then tert-butyl-tert-butyl(oxycarbonyloxy) formylate (239 mg, 1.1 mmol) was added, and the resulting mixture was stirred at room temperature for 30 min. Upon completion, the reaction mixture was diluted with water (20 mL) and extracted with ethyl acetate (30 mL×2). The combined organic layers were washed with brine (30 mL) and dried over anhydrous Na₂SO₄, filtered and concentrated under reduced pressure. The crude compound was purified by column chromatography (5-75% EtOAc in heptane) to give tert-butyl (S)-2-(3-{[(o-bromophenyl)methoxy]methyl}-5-tolyl)-4-methyl-3-(2-oxo-2,3-dihydro-1H-imidazol-1-yl)-1,2,5-triaza-2,4,6,7-tetrahydro-5H-indene-5-carboxylate (490 mg, 805 μmol) as an white solid. m/z (ESI-pos) M+1=608.3/610.4

Step H: A solution of tert-butyl (S)-2-(3-{[(o-bromophenyl)methoxy]methyl}-5-tolyl)-4-methyl-3-(2-oxo-2,3-dihydro-1H-imidazol-1-yl)-1,2,5-triaza-2,4,6,7-tetrahydro-5H-indene-5-carboxylate (490 mg, 805 μmol), copper iodide (115 mg, 604 μmol), Bathophenanthroline (268 mg, 805 μmol), and dipotassium carbonate (278 mg, 2.01 mmol) in DMSO (27 mL) was sparged with Ar for 5 minutes and then heated to 130° C. for 42 h. Upon completion, the reaction cooled to room temperature and diluted with EtOAc and sat. aq. NH₄Cl, then filtered through a pad of Celite. The filtrate was partitioned and the organic phase was washed with brine, dried with sodium sulfate, filtered, concentrated. The crude was purified by silica gel chromatography (5-50% EtOAc in heptane) to give tert-butyl (8S,10M)-8,26-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (108 mg, 205 μmol) as the major diastereomer whose stereochemistry was arbitrarily assigned. m/z (ESI-pos) M+1=528.4

Step I: Hydrogen chloride (2 mL; 4N in dioxane) was added to a vial containing tert-butyl (8S,10M)-8,26-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (91 mg, 172 μmol) and the solution stirred at room temperature for 40 min. Upon completion, the solvent was evaporated to give the crude residue, (8S,10M)-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (80 mg, 172 μmol), that was used in the next step without purification. m/z (ESI-pos) M+1=428.4

Step J: To a solution of (8S,10M)-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (80 mg, 172 μmol) in DMF (900 μL) was added 5-bromo-1-[(1S,2S)-2-methyl-1-(5-oxo-4,5-dihydro-1,2,4-oxadiazol-3-yl)cyclopropyl]-2-indolecarboxylic acid (84.8 mg, 224 μmol), N-ethyldiisopropylamine (150 μL, 862 μmol), and HATU (131 mg, 345 μmol). The resulting mixture stirred at room temperature overnight. The reaction was directly purified by Prep HPLC (Isco ACCQ Prep, Gemini column NX-C18, 250×21 mm, 20 ml/min, 45 minute separation, 20 to 80% H₂O/ACN with 0.1% Formic acid buffer), pure fractions were collected and concentrated to give (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (112 mg, 142 μmol). m/z (ESI-pos) M+1=787.4/789.4

Step K: A 2 dram vial was charged with (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (30 mg, 38.1 μmol), bromidopalladium(1+) 2-(trimethylsilyl)ethyl 1-benzenid-4-carboxylate-(3-tert-butoxy-2′,6′-diisopropyl-6-methoxy-2-biphenylyl)dicyclohexylphosphine (1/1) (3.6 mg, 3.81 μmol), sodium trimethylsilanolate (17.1 mg, 152 μmol), and (R)-2-(trifluoromethyl)morpholine-hydrogen chloride (1/1) (7.3 mg, 38.1 μmol). THF (1 mL) was added, and the mixture was sparged with Ar for 5 minutes. The vial was sealed and heated to 70° C. overnight. Upon completion, the reaction was cooled to ambient temperature, filtered through an HPLC filter, and concentrated prior to purification by Prep HPLC (Isco ACCQ Prep, Gemini column NX-C18, 250×21 mm, 20 ml/min, 45-minute separation, 20 to 80% H₂O/ACN with 0.1% Formic acid buffer). Pure fractions were collected and lyophilized to give (8S,10M)-8,26-dimethyl-7-({1-[(1S,2S)-2-methyl-1-(5-oxo-4,5-dihydro-1,2,4-oxadiazol-3-yl)cyclopropyl]-5—[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (18.1 mg, 20.9 μmol) as a white solid. m/z (ESI-pos) M+1=862.6; ¹H NMR (400 MHz, CHLOROFORM-D) δ 11.32 (s, 1H), 7.61-7.26 (m, 6H), 7.19-7.09 (m, 2H), 7.00 (d, J=13.8 Hz, 2H), 6.70 (d, J=30.6 Hz, 1H), 6.49-5.86 (m, 2H), 5.83-5.20 (m, 1H), 4.68-4.44 (m, 3H), 4.26 (dd, J=29.3, 9.4 Hz, 1H), 4.17-4.04 (m, 3H), 3.95-3.85 (m, 1H), 3.72-3.32 (m, 3H), 3.20-2.83 (m, 4H), 2.40 (d, J=8.7 Hz, 3H), 1.90 (t, J=6.2 Hz, 1H), 1.81-1.55 (m, 3H), 1.42-1.10 (m, 5H).

### Example 160—Synthesis of Compound 160

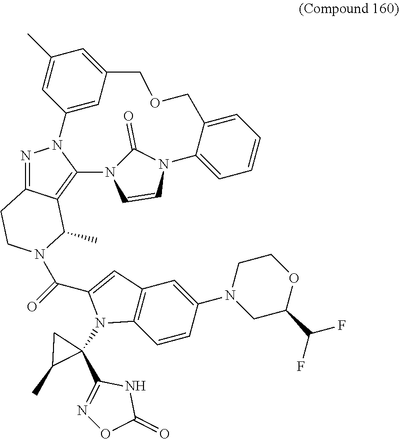

### (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 160 was synthesized following the procedure of Example 159, with (R)-2-(trifluoromethyl)morpholine-hydrogen chloride (1/1) being replaced with (R)-2-(difluoromethyl)morpholine-hydrogen chloride (1/1) in step K. m/z (ESI-pos) M+1=844.5; ¹H NMR (400 MHz, CHLOROFORM-D) δ 11.33 (s, 1H), 7.70-7.30 (m, 7H), 7.20-7.06 (m, 2H), 7.04-6.94 (m, 2H), 6.70 (d, J=30.6 Hz, 1H), 6.54-5.94 (m, 2H), 5.91-5.21 (m, 2H), 4.94-4.57 (m, 2H), 4.56-4.42 (m, 1H), 4.25 (dd, J=28.3, 9.3 Hz, 1H), 4.09 (t, J=9.8 Hz, 2H), 3.99-3.83 (m, 2H), 3.63 (td, J=13.0, 4.1 Hz, 1H), 3.43 (dd, J=54.6, 11.7 Hz, 2H), 3.10 (dtd, J=31.8, 16.2, 4.6 Hz, 2H), 2.96-2.76 (m, 2H), 2.40 (d, J=9.0 Hz, 3H), 1.96-1.75 (m, 1H), 1.65 (d, J=6.7 Hz, 3H), 1.62-1.34 (m, 1H), 1.26-1.10 (m, 3H).

### Example 161—Synthesis of Compound 161

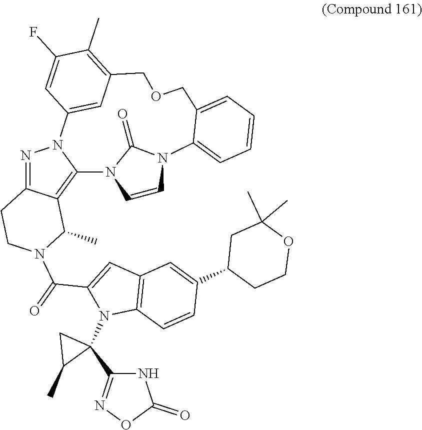

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8,25-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 161 was synthesized following the procedure of Example 69, with 2-(bromomethyl)-1-fluoro-4-iodobenzene and (o-bromophenyl)methanol and being replaced with (3-fluoro-5-iodo-2-tolyl)methanol and o-bromo (bromomethyl)benzene in step A. m/z (ESI-pos) M+1=839.5; ¹H NMR (400 MHz, CHLOROFORM-D) δ 11.31 (s, 1H), 7.64-7.25 (m, 8H), 7.06-6.91 (m, 1H), 6.84-6.68 (m, 1H), 6.54-5.89 (m, 2H), 5.84-5.22 (m, 1H), 4.96-4.41 (m, 3H), 4.34 (dd, J=28.3, 9.4 Hz, 1H), 4.17 (d, J=9.5 Hz, 1H), 3.91-3.77 (m, 1H), 3.70-3.36 (m, 1H), 3.22-2.97 (m, 3H), 2.24-2.14 (m, 3H), 1.94-1.69 (m, 4H), 1.67-1.49 (m, 7H), 1.34 (s, 3H), 1.27 (d, J=2.9 Hz, 3H), 1.16 (dd, J=28.4, 6.0 Hz, 3H).

### Example 162—Synthesis of Compound 162

### Synthetic Scheme

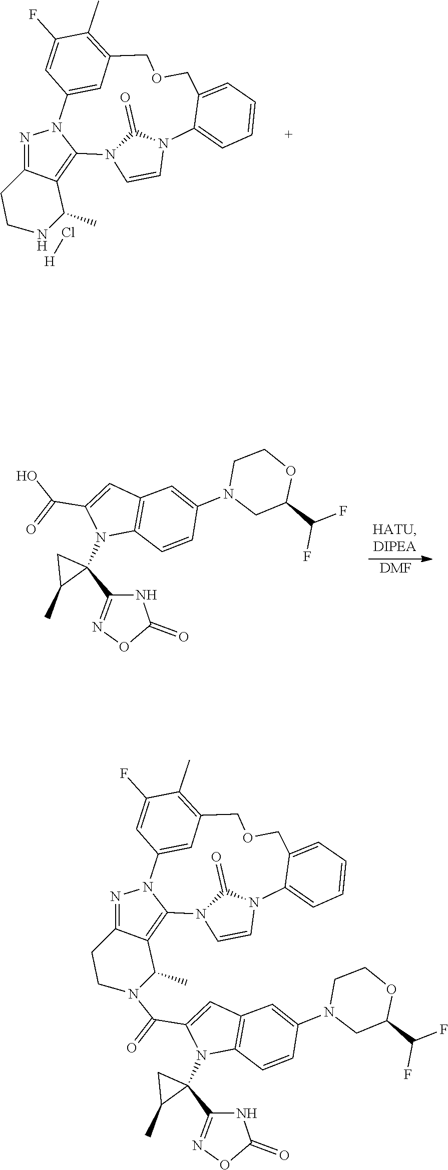

### Procedure for Synthesis of Compound 162

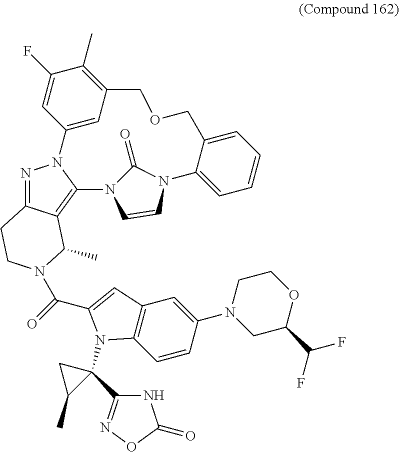

### (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-26-fluoro-8,25-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (8S,10M)-26-fluoro-8,25-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (14 mg, 29 μmol) and 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid (13.9 mg, 32 μmol) in DMF (290 μL) was added N-ethyldiisopropylamine (20.2 μL, 116 μmol) and HATU (16.6 mg, 43.6 μmol). The reaction stirred at room temperature for 4 h. The reaction mixture was directly purified by Prep HPLC (Isco ACCQ Prep, Gemini column NX-C18, 250×21 mm, 20 ml/min, 45 minute separation, 20 to 80% H₂O/ACN with 0.1% Formic acid buffer), pure fractions were collected and lyophilized to give (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-26-fluoro-8,25-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (15.3 mg, 17.8 μmol). m/z (ESI-pos) M+1=862.5; ¹H NMR (400 MHz, CHLOROFORM-D) δ 11.28 (d, J=23.3 Hz, 1H), 7.60-7.32 (m, 6H), 7.19-7.09 (m, 2H), 7.07-6.92 (m, 1H), 6.70 (d, J=32.6 Hz, 1H), 6.51-5.94 (m, 2H), 5.94-5.20 (m, 2H), 4.96-4.43 (m, 3H), 4.34 (dd, J=26.7, 9.3 Hz, 1H), 4.13 (dd, J=29.7, 10.4 Hz, 2H), 3.99-3.83 (m, 2H), 3.63 (td, J=13.0, 4.1 Hz, 1H), 3.49 (d, J=11.5 Hz, 1H), 3.36 (d, J=11.9 Hz, 1H), 3.19-2.97 (m, 2H), 2.91 (td, J=11.6, 3.3 Hz, 1H), 2.80 (t, J=10.9 Hz, 1H), 2.26-2.18 (m, 3H), 1.93-1.75 (m, 2H), 1.66 (d, J=6.7 Hz, 3H), 1.62-1.34 (m, 1H), 1.16 (dd, J=28.4, 6.1 Hz, 3H).

### Example 163—Synthesis of Compound 163

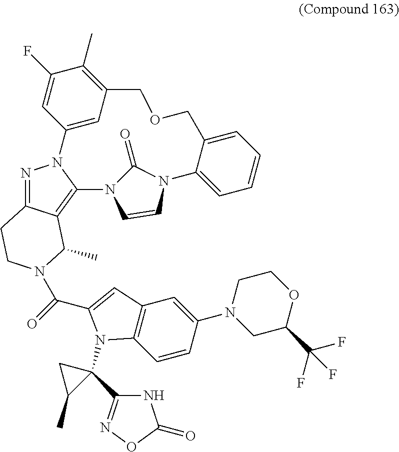

### (8S,10M)-26-fluoro-8,25-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 163 was synthesized following the procedure of Example 162, with 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid and being replaced with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid in step J. m/z (ESI-pos) M+1=880.5; ¹H NMR (400 MHz, CHLOROFORM-D) δ 11.28 (d, J=24.5 Hz, 1H), 7.64-7.43 (m, 5H), 7.38 (dd, J=10.2, 2.3 Hz, 1H), 7.21-7.07 (m, 2H), 7.06-6.91 (m, 1H), 6.80-6.63 (m, 1H), 6.52-5.88 (m, 2H), 5.88-5.18 (m, 1H), 4.95-4.42 (m, 3H), 4.40-4.27 (m, 1H), 4.21-4.08 (m, 3H), 3.90 (td, J=11.4, 2.6 Hz, 1H), 3.71-3.32 (m, 3H), 3.21-2.81 (m, 4H), 2.20 (dd, J=11.3, 1.7 Hz, 3H), 1.93-1.76 (m, 1H), 1.66 (d, J=6.7 Hz, 3H), 1.62-1.33 (m, 2H), 1.25-1.11 (m, 3H).

### Example 164—Synthesis of Compound 164

### Synthetic Scheme

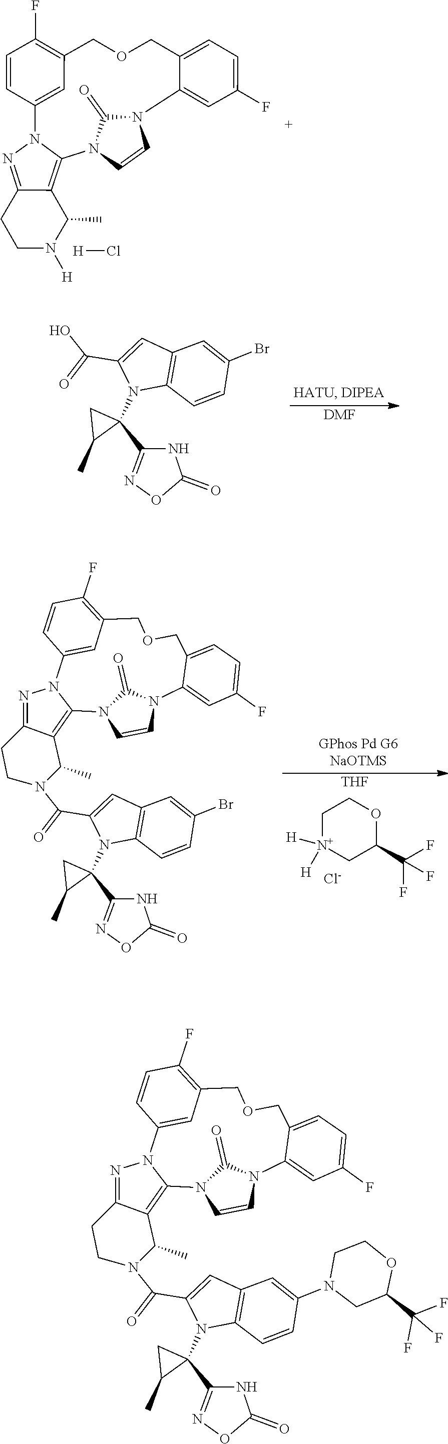

### Procedure for Synthesis of Compound 164

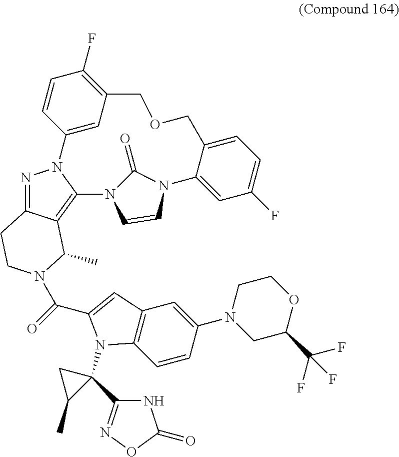

### (8S,10M)-17,25-difluoro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Step A: To a solution (8S,10M)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (22 mg, 45.3 μmol) in DMF (300 μL) was added N-ethyldiisopropylamine (39.4 μL, 226 μmol) and 5-bromo-1-[(1S,2S)-2-methyl-1-(5-oxo-4,5-dihydro-1,2,4-oxadiazol-3-yl)cyclopropyl]-2-indolecarboxylic acid (17.1 mg, 45.3 μmol) and HATU (34.4 mg, 90.5 μmol). The mixture stirred at 35° C. for 1 h. The reaction was then purified by Prep HPLC (Isco ACCQ Prep, Gemini column NX-C18, 250×21 mm, 20 ml/min, 45 minute separation, 20 to 80% H₂O/ACN with 0.1% Formic acid buffer), pure fractions were collected and concentrated to give (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (22.5 mg, 27.8 μmol). m/z (ESI-pos) M+1=809.3/811.3

Step B: A vial was charged with (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (40 mg, 49.6 μmol), bromidopalladium(1+) 2-(trimethylsilyl)ethyl 1-benzenid-4-carboxylate-(3-tert-butoxy-2′,6′-diisopropyl-6-methoxy-2-biphenylyl)dicyclohexylphosphine (1/1) (1.75 mg, 1.85 μmol), sodium trimethylsilanolate (12.5 mg, 111 μmol), (R)-2-(trifluoromethyl)morpholine-hydrogen chloride (1/1) (10.6 mg, 55.6 μmol) followed by addition of THF (1 mL). The mixture was sparged for 5 minutes then heated to 70° C. overnight. The reaction was filtered through an HPLC filter, concentrated, and purified by Prep HPLC (Isco ACCQ Prep, Gemini column NX-C18, 250×21 mm, 20 ml/min, 45-minute separation, 20 to 80% H₂O/ACN with 0.1% Formic acid buffer), pure fractions were collected and lyophilized to (8S,10M)-17,25-difluoro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (21.9 mg, 24.8 μmol). m/z (ESI-pos) M+1=884.5; ¹H NMR (400 MHz, CHLOROFORM-D) δ 11.30 (s, 1H), 7.65-7.47 (m, 3H), 7.28 (dd, J=6.1, 2.8 Hz, 1H), 7.22-7.07 (m, 6H), 6.77-6.62 (m, 1H), 6.45-5.87 (m, 2H), 5.53 (dq, J=213.8, 6.6 Hz, 1H), 4.95-4.41 (m, 3H), 4.33-4.08 (m, 4H), 3.90 (td, J=11.4, 2.7 Hz, 1H), 3.70-3.32 (m, 3H), 3.20-2.99 (m, 2H), 2.98-2.81 (m, 2H), 1.93-1.76 (m, 1H), 1.66 (d, J=6.7 Hz, 3H), 1.64-1.43 (m, 1H), 1.32-1.09 (m, 3H).

### Example 165—Synthesis of Compound 165

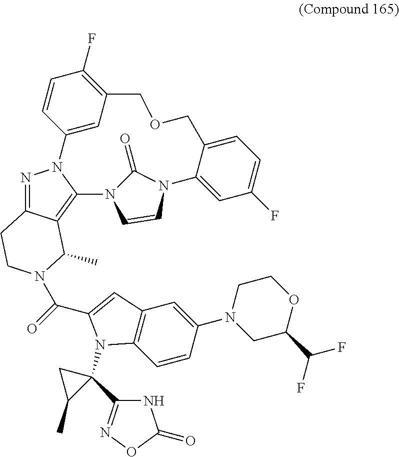

### (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 165 was synthesized following the procedure of Example 164, with (R)-2-(trifluoromethyl)morpholine-hydrogen chloride (1/1) being replaced with (R)-2-(difluoromethyl)morpholine-hydrogen chloride (1/1) in step B. m/z (ESI-pos) M+1=866.5; ¹H NMR (400 MHz, CHLOROFORM-D) δ 11.30 (s, 1H), 7.72-7.34 (m, 4H), 7.28 (dd, J=6.0, 2.8 Hz, 1H), 7.23-7.06 (m, 4H), 6.70 (d, J=34.0 Hz, 1H), 6.46-5.94 (m, 2H), 5.89-5.20 (m, 2H), 4.97-4.63 (m, 2H), 4.48 (dd, J=13.8, 5.2 Hz, 1H), 4.30-4.18 (m, 1H), 4.16-4.06 (m, 2H), 3.99-3.83 (m, 2H), 3.69-3.58 (m, 1H), 3.43 (dd, J=54.4, 11.8 Hz, 2H), 3.20-3.00 (m, 2H), 2.96-2.76 (m, 2H), 2.02-1.85 (m, 2H), 1.66 (d, J=6.7 Hz, 3H), 1.63-1.47 (m, 1H), 1.22-1.09 (m, 3H).

### Example 166—Synthesis of Compound 166

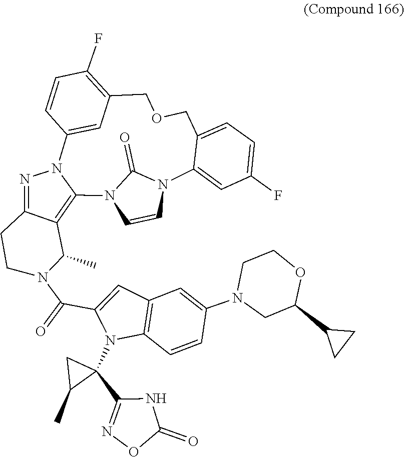

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 166 was synthesized following the procedure of Example 164, with (R)-2-(trifluoromethyl)morpholine-hydrogen chloride (1/1) being replaced(S)-2-cyclopropylmorpholine in step B. m/z (ESI-pos) M+1=856.6; ¹H NMR (400 MHz, CHLOROFORM-D) δ 11.31 (s, 1H), 7.79-7.44 (m, 3H), 7.28 (dd, J=6.1, 2.8 Hz, 1H), 7.22-7.08 (m, 5H), 6.79-6.59 (m, 1H), 6.48-5.85 (m, 2H), 5.84-5.20 (m, 1H), 4.94-4.42 (m, 3H), 4.29-4.02 (m, 3H), 3.79 (td, J=11.4, 2.6 Hz, 1H), 3.63 (td, J=13.1, 4.1 Hz, 1H), 3.52 (dt, J=11.5, 2.1 Hz, 1H), 3.34 (d, J=11.7 Hz, 1H), 3.20-2.99 (m, 2H), 2.98-2.80 (m, 2H), 2.71 (dd, J=11.6, 10.1 Hz, 1H), 1.86 (dt, J=24.6, 6.3 Hz, 1H), 1.72 (dd, J=53.1, 6.7 Hz, 3H), 1.62-1.23 (m, 2H), 1.23-1.08 (m, 3H), 1.02-0.83 (m, 1H), 0.67-0.40 (m, 3H), 0.29 (dq, J=9.6, 4.7 Hz, 1H).

### Example 167—Synthesis of Compound 167

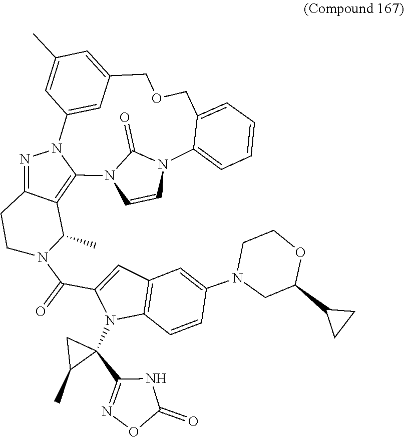

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 167 was synthesized following the procedure of Example 159, with (R)-2-(trifluoromethyl)morpholine-hydrogen chloride (1/1) being replaced with(S)-2-cyclopropylmorpholine in step K. m/z (ESI-pos) M+1=834.6; ¹H NMR (400 MHz, CHLOROFORM-D) δ 11.34 (s, 1H), 7.65-7.28 (m, 6H), 7.19-7.10 (m, 2H), 7.04-6.94 (m, 2H), 6.78-6.63 (m, 1H), 6.49-5.86 (m, 2H), 5.85-5.19 (m, 1H), 5.00-4.42 (m, 3H), 4.25 (dd, J=28.1, 9.3 Hz, 1H), 4.13-4.01 (m, 2H), 3.79 (td, J=11.4, 2.6 Hz, 1H), 3.63 (td, J=13.0, 4.1 Hz, 1H), 3.52 (d, J=11.5 Hz, 1H), 3.35 (d, J=11.7 Hz, 1H), 3.20-3.00 (m, 2H), 2.98-2.81 (m, 2H), 2.76-2.66 (m, 1H), 2.40 (d, J=9.1 Hz, 3H), 1.86 (dt, J=23.9, 6.2 Hz, 1H), 1.72 (dd, J=52.9, 6.7 Hz, 3H), 1.61-1.46 (m, 1H), 1.16 (dd, J=29.8, 6.1 Hz, 4H), 0.95 (tq, J=8.7, 4.2 Hz, 1H), 0.58 (dtt, J=21.7, 8.7, 4.1 Hz, 2H), 0.46 (dt, J=9.5, 4.7 Hz, 1H), 0.29 (dq, J=9.5, 4.8 Hz, 1H).

### Example 168—Synthesis of Compound 168

### Synthetic Scheme

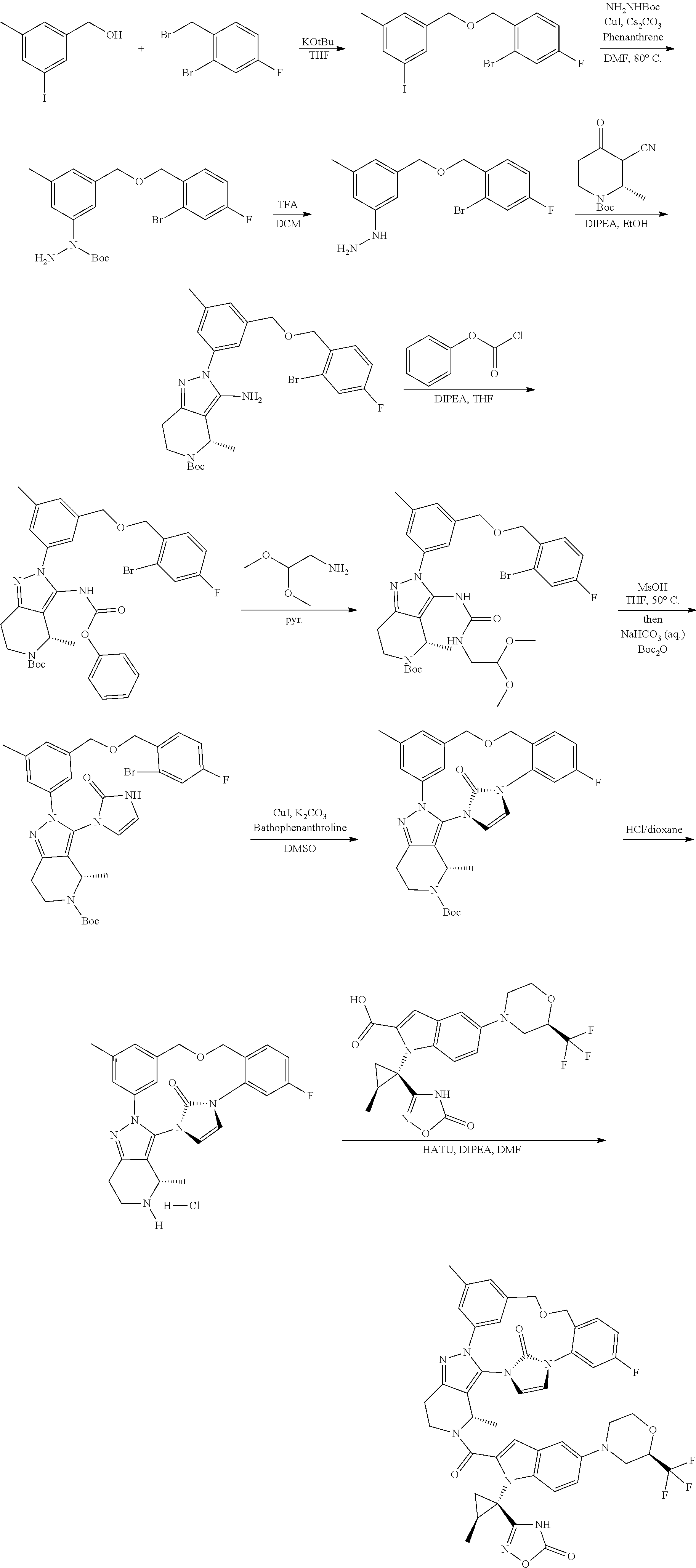

### Procedure for Synthesis of Compound 168

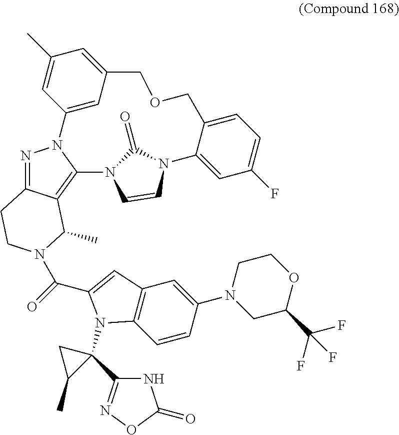

### (8S,10M)-17-fluoro-8,26-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Step A: Potassium t-butoxide (1.45 g, 12.9 mmol) added to an ice-bath cooled solution of (3-iodo-5-tolyl)methanol (2.92 g, 11.8 mmol) in THF (29.7 mL). The solution was stirred for 5 min before 2-bromo-1-(bromomethyl)-4-fluorobenzene (3.15 g, 11.8 mmol) was added After 30 min, the ice-bath was removed and the reaction was allowed to warm to room temperature overnight. Upon completion, the reaction mixture was diluted with water and heptane, and the layers were separated. The organics were dried over Na₂SO₄ and concentrated. The crude oil, 2-bromo-4-fluoro-1-{[(3-iodo-5-tolyl)methoxy]methyl}benzene (5.1 g, 11.7 mmol) was used in the following step without purification. m/z (ESI-pos) M+1=not found—does not ionize

Step B: A solution of 2-bromo-4-fluoro-1-{[(3-iodo-5-tolyl)methoxy]methyl}benzene (1.12 g, 2.57 mmol), tert-butyl carbazate (442 mg, 3.35 mmol), dicaesium carbonate (1.17 g, 3.6 mmol), copper iodide (24.5 mg, 129 μmol), and 4,5-diazaphenanthrene (92.8 mg, 515 μmol) in DMF (2.6 mL) was sparged with Ar for 5 min and then heated to 80° C. for 3 h. Upon completion, EtOAc (20 mL) was added, and the mixture was filtered through Celite, and the filtrate was washed with brine (3×20 mL). The organics were dried over Na₂SO₄, filtered, and concentrated. The crude product purified by silica gel chromatography (5-30% EtOAc in heptane) to give tert-butyl 2-(3-{[(2-bromo-4-fluorophenyl)methoxy]methyl}-5-tolyl)carbazate (880 mg, 2 mmol) as a colorless oil. m/z (ESI-pos) M+1-tBu=383.1/385.2

Step C: To a solution of tert-butyl 2-(3-{[(2-bromo-4-fluorophenyl)methoxy]methyl}-5-tolyl)carbazate (880 mg, 2 mmol) in DCM (6.7 mL) was added TFA (2.3 mL, 30 mmol) at room temperature. After 3 h, the reaction was *carefully* quenched with sat. aq. Na₂CO₃ (~30 mL), then extracted DCM (2×20 mL). The combined organic layers were washed with sat. aq. NaHCO₃ (3×20 mL), water (2×20 mL) and brine (1×20 mL), and then dried over Na₂SO₄, filtered, and concentrated. The crude (3-{[(2-bromo-4-fluorophenyl)methoxy]methyl}-5-tolyl) hydrazine (595 mg, 1.75 mmol) was used without purification in the following step. m/z (ESI-pos) M+1=339.1/341.1

Step D: To a solution of crude (3-{[(2-bromo-4-fluorophenyl)methoxy]methyl}-5-tolyl) hydrazine (595 mg, 1.75 mmol) in EtOH (7.02 mL) was added N-ethyldiisopropylamine (306 μL, 1.75 mmol) and tert-butyl(2S)-3-cyano-2-methyl-4-oxo-1-piperidinecarboxylate (502 mg, 2.1 mmol) at room temperature. The mixture was stirred at 80° C. for 24 h. After completion, the reaction mixture was cooled and evaporated under reduced pressure to give the crude compound which was purified by column chromatography (10-60% EtOAc in heptane) to give tert-butyl (S)-3-amino-2-(3-{[(2-bromo-4-fluorophenyl)methoxy]methyl}-5-tolyl)-4-methyl-1,2,5-triaza-2,4,6,7-tetrahydro-5H-indene-5-carboxylate (491 mg, 878 μmol) as a yellow solid over 2 steps. m/z (ESI-pos) M+1=559.3/561.3

Step E: To a solution of tert-butyl (S)-3-amino-2-(3-{[(2-bromo-4-fluorophenyl)methoxy]methyl}-5-tolyl)-4-methyl-1,2,5-triaza-2,4,6,7-tetrahydro-5H-indene-5-carboxylate (491 mg, 878 μmol) in THF (4.4 mL) was added N-ethyldiisopropylamine (459 μL, 2.63 mmol) followed by benzenyl chloroformate (220 μL, 1.76 mmol). The reaction mixture was stirred at 25° C. for 30 min. Upon completion, the reaction was directly concentrated under reduced pressure to give the crude tert-butyl (S)-2-(3-{[(2-bromo-4-fluorophenyl)methoxy]methyl}-5-tolyl)-4-methyl-3-[phenyl(oxycarbonylamino)]-1,2,5-triaza-2,4,6,7-tetrahydro-5H-indene-5-carboxylate (596 mg, 877 μmol) as a yellow solid, which was used directly in the next step without any further purification. m/z (ESI-pos) M+1=679.4/681.3

Step F: Crude tert-butyl (S)-2-(3-{[(2-bromo-4-fluorophenyl)methoxy]methyl}-5-tolyl)-4-methyl-3-[phenyl(oxycarbonylamino)]-1,2,5-triaza-2,4,6,7-tetrahydro-5H-indene-5-carboxylate (596 mg, 877 μmol) was dissolved in pyridine (2.92 mL) and then 2,2-dimethoxyethylamine (143 μL, 1.32 mmol) was added. The mixture was stirred at room temperature for 30 min and then directly concentrated under reduced pressure. The crude residue was purified by column chromatography (5-80% EtOAc in heptane) to give tert-butyl (S)-2-(3-{[(2-bromo-4-fluorophenyl)methoxy]methyl}-5-tolyl)-3-[3-(2,2-dimethoxyethyl)ureido]-4-methyl-1,2,5-triaza-2,4,6,7-tetrahydro-5H-indene-5-carboxylate (440 mg, 637 μmol) as a white solid over 2 steps. m/z (ESI-pos) M+1=690.4/692.4

Step G: To a solution of tert-butyl (S)-2-(3-{[(2-bromo-4-fluorophenyl)methoxy]methyl}-5-tolyl)-3-[3-(2,2-dimethoxyethyl)ureido]-4-methyl-1,2,5-triaza-2,4,6,7-tetrahydro-5H-indene-5-carboxylate (440 mg, 637 μmol) in THF (3.19 mL) was added methanesulfonic acid (124 μL, 1.91 mmol). The mixture was stirred at 55° C. for 1 h and then cooled to ambient temperature. Saturated aqueous NaHCO₃ was added until pH 8 then tert-butyl-tert-butyl(oxycarbonyloxy) formylate (153 mg, 701 μmol) was added, and the resulting mixture was stirred at room temperature for 30 min. The reaction was diluted with water (20 mL) and extracted with ethyl acetate (30 mL×2). The combined organic layers were washed with brine (30 mL) and dried over anhydrous Na₂SO₄, filtered and concentrated under reduced pressure to give the crude compound, which was purified by column chromatography (5-75% EtOAc in heptane) to give tert-butyl (S)-2-(3-{[(2-bromo-4-fluorophenyl)methoxy]methyl}-5-tolyl)-4-methyl-3-(2-oxo-2,3-dihydro-1H-imidazol-1-yl)-1,2,5-triaza-2,4,6,7-tetrahydro-5H-indene-5-carboxylate (328 mg, 524 μmol) as a white solid. m/z (ESI-pos) M+1=626.3/628.3

Step H: A solution of tert-butyl (S)-2-(3-{[(2-bromo-4-fluorophenyl)methoxy]methyl}-5-tolyl)-4-methyl-3-(2-oxo-2,3-dihydro-1H-imidazol-1-yl)-1,2,5-triaza-2,4,6,7-tetrahydro-5H-indene-5-carboxylate (1.3 g, 2.07 mmol), copper iodide (296 mg, 1.56 mmol), bathophenanthroline (690 mg, 2.07 mmol), and dipotassium carbonate (860 mg, 6.22 mmol) in DMSO (63 mL) was sparged with Ar for 30 minutes, and then heated to 130° C. under Ar balloon for 70 h. The reaction was diluted with EtOAc and sat. aq. NH₄Cl, the workup solution (organic and aqueous) was filtered through a pad of celite. The filtrate was partitioned and the organic phase was washed with brine, dried with sodium sulfate, filtered, concentrated. The crude was purified by silica gel chromatography (5-50% EtOAc in heptane) to give tert-butyl (8S,10M)-17-fluoro-8,26-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (0.2 g, 367 μmol) as the major diastereomer whose stereochemistry was arbitrarily assigned. m/z (ESI-pos) M+1=546.4

Step I: Hydrogen chloride (4N in dioxane; 1.5 mL) was added to a vial containing tert-butyl (8S,10M)-17-fluoro-8,26-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (33 mg, 60.5 μmol) and the solution stirred at room temperature for 30 min. Upon completion, the solvent was evaporated to give the crude residue (8S,10M)-17-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (29 mg, 60.2 μmol) that was used in the next step without purification. m/z (ESI-pos) M+1=446.3

Step J: To a solution of (8S,10M)-17-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (14 mg, 29 μmol) and 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid (14.5 mg, 32 μmol) in DMF (290 μL) was added N-ethyldiisopropylamine (20.2 μL, 116 μmol) and HATU (16.6 mg, 43.6 μmol). The reaction was stirred at room temperature overnight. The crude reaction mixture was directly purified by Prep HPLC (Isco ACCQ Prep, Gemini column NX-C18, 250×21 mm, 20 mL/min, 45 minute separation, 20 to 80% H₂O/ACN with 0.1% Formic acid buffer), pure fractions were collected and lyophilized to give (8S,10M)-17-fluoro-8,26-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (17.8 mg, 20.2 μmol). m/z (ESI-pos) M+1=880.6; ¹H NMR (400 MHz, CHLOROFORM-D) δ 11.32 (s, 1H), 7.57 (t, J=8.5 Hz, 1H), 7.51-7.40 (m, 2H), 7.20-7.04 (m, 5H), 7.04-6.91 (m, 2H), 6.70 (d, J=32.1 Hz, 1H), 6.50-5.85 (m, 2H), 5.86-5.21 (m, 1H), 4.97-4.41 (m, 3H), 4.24 (d, J=9.5 Hz, 1H), 4.19-4.02 (m, 3H), 3.95-3.85 (m, 1H), 3.68-3.50 (m, 2H), 3.36 (d, J=12.1 Hz, 1H), 3.20-3.00 (m, 2H), 2.98-2.82 (m, 2H), 2.40 (d, J=9.0 Hz, 3H), 1.94-1.75 (m, 1H), 1.65 (d, J=6.7 Hz, 3H), 1.59-1.47 (m, 1H), 1.21-1.03 (m, 3H).

### Example 169—Synthesis of Compound 169

### Synthetic Scheme

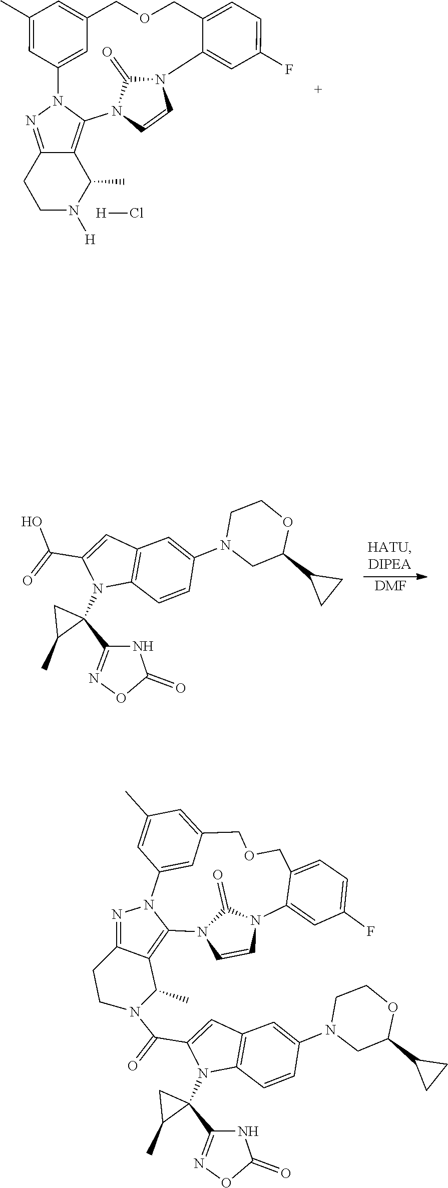

### Procedure for Synthesis of Compound 169

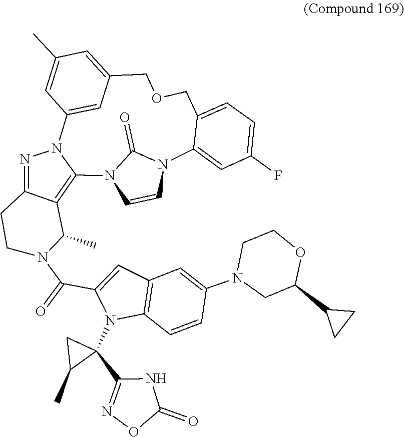

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (8S,10M)-17-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (10 mg, 20.7 μmol) and 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid (9.69 mg, 22.8 μmol) in DMF (200 μL) was added N-ethyldiisopropylamine (15 μL, 83 μmol) and HATU (11.8 mg, 31.1 μmol). The reaction stirred at room temperature overnight. The crude reaction was directly purified by Prep HPLC (Isco ACCQ Prep, Gemini column NX-C18, 250×21 mm, 20 mL/min, 45 minute separation, 20 to 80% H₂O/ACN with 0.1% Formic acid buffer), pure fractions were collected and lyophilized to give (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (4.9 mg, 5.75 μmol). m/z (ESI-pos) M+1=852.6; ¹H NMR (400 MHz, CHLOROFORM-D) δ 11.28 (d, J=22.9 Hz, 1H), 7.61-7.41 (m, 3H), 7.24-6.93 (m, 6H), 6.67 (s, 1H), 6.48-5.87 (m, 2H), 5.86-5.25 (m, 1H), 4.96-4.43 (m, 3H), 4.21 (dd, J=29.3, 9.5 Hz, 1H), 4.05 (dd, J=9.6, 6.0 Hz, 2H), 3.83 (t, J=11.4 Hz, 1H), 3.68-3.57 (m, 1H), 3.52 (s, 1H), 3.37 (d, J=11.6 Hz, 1H), 3.21-2.73 (m, 5H), 2.40 (d, J=8.8 Hz, 3H), 1.93-1.77 (m, 1H), 1.65 (d, J=6.7 Hz, 3H), 1.63-1.47 (m, 1H), 1.41-1.08 (m, 4H), 1.00-0.83 (m, 1H), 0.68-0.50 (m, 2H), 0.47 (dq, J=9.4, 4.7 Hz, 1H), 0.31 (dt, J=9.4, 4.7 Hz, 1H).

### Example 170—Synthesis of Compound 170

### Synthetic Scheme

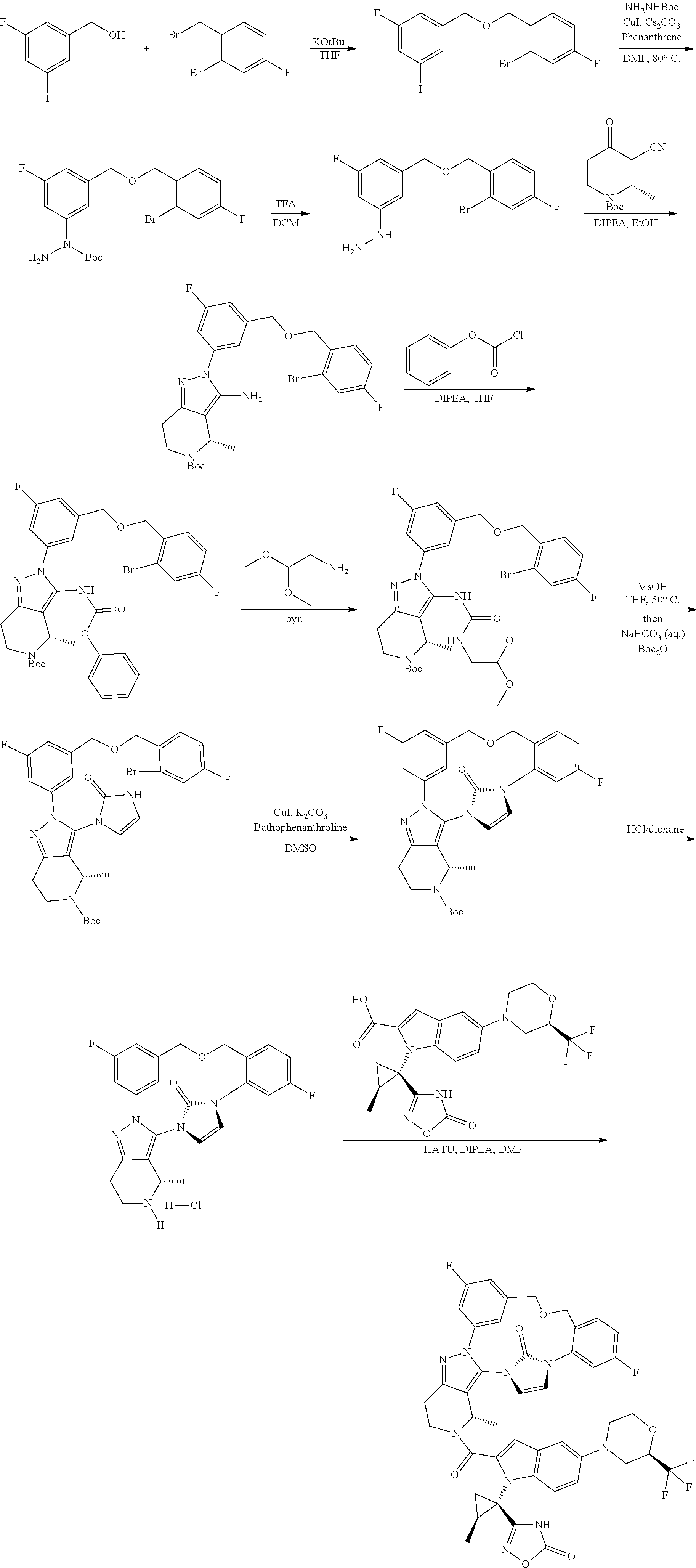

### Procedure for Synthesis of Compound 170

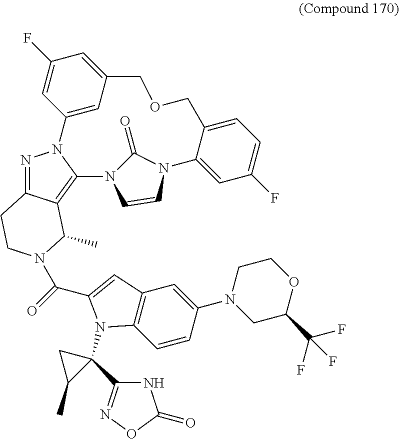

### (8S,10M)-17,26-difluoro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: Potassium t-butoxide (2.45 g, 21.8 mmol) was added to an ice-bath cooled solution of (3-fluoro-5-iodophenyl)methanol (5 g, 19.8 mmol) in THF (50 mL). The reaction was stirred for 5 min before 2-bromo-1-(bromomethyl)-4-fluorobenzene (5.32 g, 19.8 mmol) was added. The ice bath was removed, and reaction was allowed to warm to room temperature for 1.5 h. Upon completion, the reaction mixture was diluted with H₂O (50 mL) and heptane (100 mL) and the layers were separated. The organics were dried over Na₂SO₄, filtered, and concentrated. The crude oil, 2-bromo-4-fluoro-1-{[(3-fluoro-5-iodophenyl)methoxy]methyl}benzene (7.8 g, 17.8 mmol), was used in the following step without purification. m/z (ESI-pos) M+1=not found-does not ionize

Step B: A solution of 1-bromo-4-fluoro-2-{[(3-fluoro-5-iodophenyl)methoxy]methyl}benzene (1.21 g, 2.76 mmol), tert-butyl carbazate (474 mg, 3.58 mmol), dicaesium carbonate (1.26 g, 3.86 mmol), copper iodide (26.2 mg, 138 μmol), and 4,5-diazaphenanthrene (99.3 mg, 551 μmol) in DMF (2.76 mL) was sparged with Ar for 5 min and then heated to 80° C. for 3 h. Upon completion, EtOAc (20 mL) was added and the mixture was filtered through Celite, and the filtrate was washed with brine (3×20 mL). The organics were dried over Na₂SO₄, filtered, and concentrated. The crude product was purified by silica gel chromatography (5-30% EtOAc in heptane) to give tert-butyl 2-(m-{[2-(2-bromo-4,5-difluorophenyl)ethoxy]methyl}phenyl)carbazate (347 mg, 759 μmol) as a colorless oil. m/z (ESI-pos) M+1-tBu=387.1/389.1

Step C: To a solution of tert-butyl 2-(5-{[(2-bromo-4-fluorophenyl)methoxy]methyl}-3-fluorophenyl)carbazate (975 mg, 2.2 mmol) in DCM (7.33 mL), trifluoroacetic acid (2.52 mL, 33 mmol) was added at room temperature. The reaction was stirred for 2 h. The reaction was then *carefully* quenched with sat. aq. Na₂CO₃ (~30 mL), then extracted DCM (2×20 mL). The combined organic layers were washed with sat. aq. NaHCO₃ (3×20 mL), water (2×20 mL) and brine (1×20 mL), and then dried over Na₂SO₄, filtered, and concentrated. The crude (5-{[(2-bromo-4-fluorophenyl)methoxy]methyl}-3-fluorophenyl)hydrazine (0.6 g, 1.75 mmol) was used without purification in the following step. m/z (ESI-pos) M+1=343.0/345.1

Step D: To a solution of crude (5-{[(2-bromo-4-fluorophenyl)methoxy]methyl}-3-fluorophenyl)hydrazine (0.6 g, 1.75 mmol) in EtOH (7 mL) was added N-ethyldiisopropylamine (305 μL, 1.75 mmol) and tert-butyl(2S)-3-cyano-2-methyl-4-oxo-1-piperidinecarboxylate (0.5 g, 2.1 mmol). The mixture was stirred at 80° C. for 20 h. After completion, the reaction mixture was cooled to room temperature and the volatiles evaporated under reduced pressure. The crude compound was purified by column chromatography (15-60% EtOAc in heptane) to give tert-butyl (S)-3-amino-2-(5-{[(2-bromo-4-fluorophenyl)methoxy]methyl}-3-fluorophenyl)-4-methyl-1,2,5-triaza-2,4,6,7-tetrahydro-5H-indene-5-carboxylate (649 mg, 1.15 mmol) as a yellow solid over 2 steps. m/z (ESI-pos) M+1=563.3/565.3

Step E: To a solution of tert-butyl (S)-3-amino-2-(5-{[(2-bromo-4-fluorophenyl)methoxy]methyl}-3-fluorophenyl)-4-methyl-1,2,5-triaza-2,4,6,7-tetrahydro-5H-indene-5-carboxylate (649 mg, 1.15 mmol) in THF (5.76 mL) was added N-ethyldiisopropylamine (602 μL, 3.46 mmol) followed by benzenyl chloroformate (289 μL, 2.3 mmol). The reaction mixture was stirred at 25° C. for 30 min. Upon completion, the reaction was directly concentrated under reduced pressure to give the crude tert-butyl (S)-2-(5-{[(2-bromo-4-fluorophenyl)methoxy]methyl}-3-fluorophenyl)-4-methyl-3-[phenyl(oxycarbonylamino)]-1,2,5-triaza-2,4,6,7-tetrahydro-5H-indene-5-carboxylate (787 mg, 1.15 mmol) which was used directly in the next step without any further purification. m/z (ESI-pos) M+1=683.3/685.3

Step F: Crude tert-butyl (S)-2-(5-{[(2-bromo-4-fluorophenyl)methoxy]methyl}-3-fluorophenyl)-4-methyl-3-[phenyl(oxycarbonylamino)]-1,2,5-triaza-2,4,6,7-tetrahydro-5H-indene-5-carboxylate (787 mg, 1.15 mmol) was dissolved in pyridine (3.84 mL) and then 2,2-dimethoxyethylamine (188 μL, 1.73 mmol) was added. The mixture was stirred at room temperature for 30 min. Upon completion, the reaction was concentrated under reduced pressure to give the crude residue which was purified by column chromatography (5-80% EtOAc in heptane) to give tert-butyl (S)-2-(5-{[(2-bromo-4-fluorophenyl)methoxy]methyl}-3-fluorophenyl)-3-[3-(2,2-dimethoxyethyl)ureido]-4-methyl-1,2,5-triaza-2,4,6,7-tetrahydro-5H-indene-5-carboxylate (422 mg, 608 μmol) as a white solid over 2 steps. m/z (ESI-pos) M+1=694.4/696.3

Step G: To a solution of tert-butyl (S)-2-(5-{[(2-bromo-4-fluorophenyl)methoxy]methyl}-3-fluorophenyl)-3-[3-(2,2-dimethoxyethyl)ureido]-4-methyl-1,2,5-triaza-2,4,6,7-tetrahydro-5H-indene-5-carboxylate (422 mg, 608 μmol) in THF (3.04 mL, 37.3 mmol) was added methanesulfonic acid (118 μL, 1.82 mmol). The mixture was stirred at 55° C. for 1 h and cooled to ambient temperature. Saturated aqueous NaHCO₃ was added until pH 8 then tert-butoxycarbonyl-tert-butyl carbonate (146 mg, 668 μmol) was added, and the resulting mixture was stirred at room temperature for 30 min. The reaction was diluted with water (20 mL) and extracted with ethyl acetate (30 mL×2). The combined organic layers were washed with brine (30 mL) and dried over anhydrous Na₂SO₄, filtered and concentrated under reduced pressure. The crude compound was purified by column chromatography (5-90% EtOAc in heptane) to give tert-butyl (S)-2-(5-{[(2-bromo-4-fluorophenyl)methoxy]methyl}-3-fluorophenyl)-4-methyl-3-(2-oxo-2,3-dihydro-1H-imidazol-1-yl)-1,2,5-triaza-2,4,6,7-tetrahydro-5H-indene-5-carboxylate (327 mg, 519 μmol) as an off-white solid. m/z (ESI-pos) M+1=630.3/632.3

Step H: A solution of tert-butyl (S)-2-(5-{[(2-bromo-4-fluorophenyl)methoxy]methyl}-3-fluorophenyl)-4-methyl-3-(2-oxo-2,3-dihydro-1H-imidazol-1-yl)-1,2,5-triaza-2,4,6,7-tetrahydro-5H-indene-5-carboxylate (327 mg, 519 μmol), copper iodide (74.1 mg, 389 μmol), Bathophenanthroline (172 mg, 519 μmol), and dipotassium carbonate (179 mg, 1.3 mmol) in DMSO (17.3 mL) was sparged with Ar for 5 minutes, and then heated to 130° C. in a sealed vial for 2 days. The reaction was diluted with EtOAc and sat. aq. NH₄Cl, the workup solution (organic and aqueous) was filtered through a pad of celite. The filtrate was partitioned and the organic phase was washed with brine, dried over sodium sulfate, filtered, concentrated. The crude was purified by silica gel chromatography (5-50% EtOAc in heptane) to give tert-butyl (8S,10M)-17,26-difluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (74.2 mg, 135 μmol) as the major diastereomer whose stereochemistry was arbitrarily assigned. m/z (ESI-pos) M+1=550.4

Step I: Hydrogen chloride (2 mL; 4N in dioxane) was added to a vial containing tert-butyl (8S,10M)-17,26-difluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (53.8 mg, 97.9 μmol) and the solution stirred at room temperature for 60 min. Upon completion, the solvent was evaporated to give the crude residue, (8S,10M)-17,26-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (48 mg, 98.8 μmol), that was used in the next step without purification. m/z (ESI-pos) M+1=450.3

Step J: To a solution of (8S,10M)-17,26-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (14.5 mg, 29.8 μmol) and 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid (14.8 mg, 32.8 μmol) in DMF (300 μL) was added HATU (17 mg, 44.8 μmol) and N-ethyldiisopropylamine (20.8 μL, 119 μmol). The mixture stirred at room temperature for 2 days. The crude reaction was directly purified by Prep HPLC (Isco ACCQ Prep, Gemini column NX-C18, 250×21 mm, 20 mL/min, 45 minute separation, 20 to 80% H2O/ACN with 0.1% Formic acid buffer), pure fractions were collected and lyophilized to give (8S,10M)-17,26-difluoro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (20.4 mg, 23.1 μmol). m/z (ESI-pos) M+1=884.5; 11.28 (s, 1H), 7.65-7.53 (m, 1H), 7.52-7.35 (m, 2H), 7.22-7.04 (m, 4H), 6.97-6.86 (m, 2H), 6.70 (d, J=32.6 Hz, 1H), 6.54-5.92 (m, 2H), 5.85-5.22 (m, 1H), 4.99-4.44 (m, 3H), 4.28 (dd, J=30.4, 9.4 Hz, 1H), 4.19-4.10 (m, 2H), 4.05 (d, J=9.6 Hz, 1H), 3.90 (td, J=11.4, 2.5 Hz, 1H), 3.69-3.50 (m, 2H), 3.36 (d, J=11.9 Hz, 1H), 3.20-3.00 (m, 2H), 3.00-2.82 (m, 2H), 2.00 (s, 1H), 1.90 (t, J=6.1 Hz, 1H), 1.64 (d, J=6.7 Hz, 3H), 1.62-1.46 (m, 1H), 1.21-1.09 (m, 3H).

### Example 171—Synthesis of Compound 171

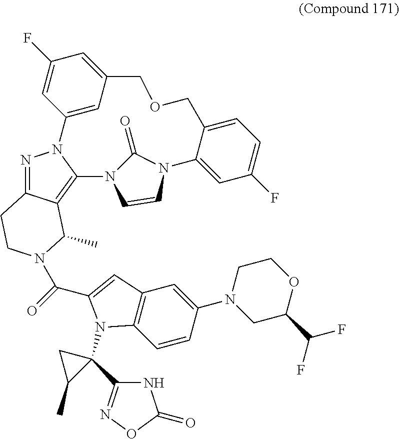

### (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17,26-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 171 was synthesized following the procedure of Example 170, with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid being replaced with 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid in step J. m/z (ESI-pos) M+1=866.5; ¹H NMR (400 MHz, CHLOROFORM-D) δ 11.27 (d, J=26.2 Hz, 1H), 7.62-7.37 (m, 3H), 7.22-7.06 (m, 4H), 7.00-6.88 (m, 2H), 6.71 (d, J=32.5 Hz, 1H), 6.54-5.96 (m, 2H), 5.96-5.25 (m, 2H), 5.01-4.45 (m, 3H), 4.30 (dd, J=30.5, 9.4 Hz, 1H), 4.08 (dd, J=20.8, 10.4 Hz, 2H), 4.00-3.84 (m, 2H), 3.64 (td, J=13.1, 4.1 Hz, 1H), 3.51 (d, J=11.5 Hz, 41H), 3.37 (d, J=11.9 Hz, 1H), 3.21-3.01 (m, 2H), 2.92 (td, J=11.6, 3.3 Hz, 1H), 2.82 (t, J=10.9 Hz, 1H), 2.04-1.76 (m, 2H), 1.59 (dd, J=44.7, 6.1 Hz, 4H), 1.26-1.11 (m, 3H).

### Example 172—Synthesis of Compound 172

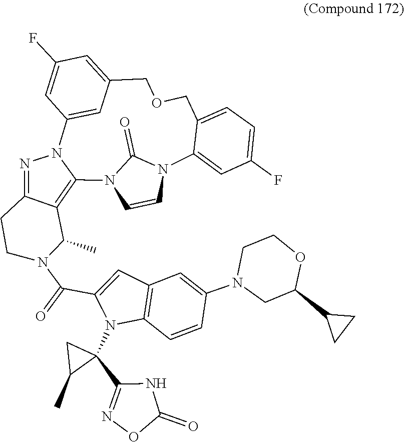

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17,26-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 172 was synthesized following the procedure of Example 170, with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid being replaced with 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid in step J. m/z (ESI-pos) M+1=856.6; ¹H NMR (400 MHz, CHLOROFORM-D) δ 11.28 (d, J=26.6 Hz, 1H), 7.75-7.34 (m, 3H), 7.23-7.07 (m, 4H), 6.97-6.86 (m, 2H), 6.70 (d, J=36.0 Hz, 1H), 6.56-5.91 (m, 2H), 5.86-5.22 (m, 1H), 4.72-4.44 (m, 3H), 4.28 (dd, J=29.7, 9.5 Hz, 1H), 4.05 (d, J=9.9 Hz, 2H), 3.83-3.73 (m, 1H), 3.62 (td, J=13.1, 4.2 Hz, 1H), 3.43 (dd, J=69.0, 11.6 Hz, 2H), 3.22-2.81 (m, 3H), 2.71 (t, J=10.9 Hz, 1H), 2.06-1.73 (m, 3H), 1.64 (dd, J=6.7, 4.0 Hz, 3H), 1.58-1.34 (m, 1H), 1.29-1.08 (m, 4H), 0.95 (tq, J=8.4, 4.2 Hz, 1H), 0.71-0.24 (m, 3H).

### Example 173—Synthesis of Compound 173

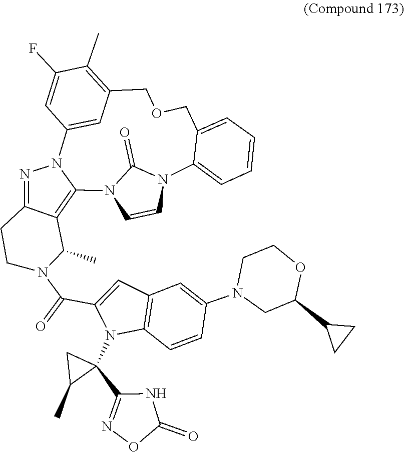

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-26-fluoro-8,25-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 173 was synthesized following the procedure of Example 162, with 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid in step A. m/z (ESI-pos) M+1=852.6; ¹H NMR (400 MHz, CHLOROFORM-D) δ 11.29 (d, J=22.5 Hz, 1H), 7.67-7.32 (m, 6H), 7.14 (d, J=8.4 Hz, 2H), 7.07-6.90 (m, 1H), 6.65 (s, 1H), 6.50-5.88 (m, 2H), 5.86-5.22 (m, 1H), 4.95-3.99 (m, 6H), 3.90-3.58 (m, 2H), 3.43 (dd, J=68.4, 11.7 Hz, 2H), 3.21-2.79 (m, 3H), 2.72 (t, J=10.8 Hz, 1H), 2.24-2.17 (m, 3H), 1.94-1.82 (m, 1H), 1.72 (dd, J=52.2, 6.7 Hz, 3H), 1.59-1.32 (m, 3H), 1.16 (dd, J=28.8, 6.1 Hz, 4H), 0.94 (tt, J=8.5, 4.7 Hz, 1H), 0.65-0.42 (m, 2H), 0.29 (dq, J=9.7, 4.8 Hz, 1H).

### Example 174—Synthesis of Compound 174

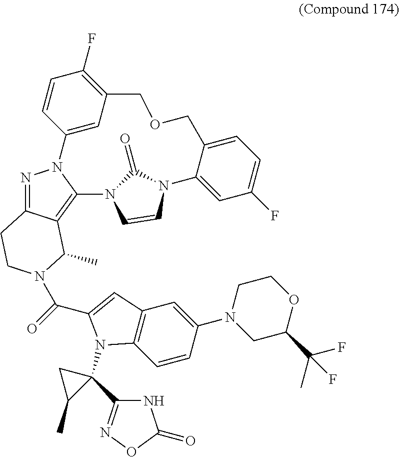

### (8S,10M)-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 174 was synthesized following the procedure of Example 164, with (R)-2-(trifluoromethyl)morpholine-hydrogen chloride (1/1) being replaced (R)-2-(1,1-difluoroethyl) morpholine in step B. m/z (ESI-pos) M+1=880.6; ¹H NMR (400 MHz, CHLOROFORM-D) δ 11.30 (s, 1H), 7.65-7.47 (m, 3H), 7.25 (s, 3H), 7.23-7.07 (m, 5H), 6.69 (d, J=34.2 Hz, 1H), 6.49-5.88 (m, 2H), 5.53 (dq, J=210.9, 6.7 Hz, 1H), 4.98-4.63 (m, 2H), 4.48 (dd, J=13.8, 5.0 Hz, 1H), 4.30-4.18 (m, 1H), 4.16-4.06 (m, 2H), 3.87 (td, J=11.2, 2.4 Hz, 1H), 3.69-3.53 (m, 2H), 3.37 (d, J=11.9 Hz, 1H), 3.20-2.99 (m, 2H), 2.87 (td, J=11.8, 3.3 Hz, 1H), 2.78 (t, J=11.1 Hz, 1H), 1.92-1.77 (m, 2H), 1.75-1.63 (m, 5H), 1.61-1.47 (m, 1H), 1.16 (dd, J=30.4, 6.1 Hz, 3H).

### Example 175—Synthesis of Compound 175

### Synthetic Scheme

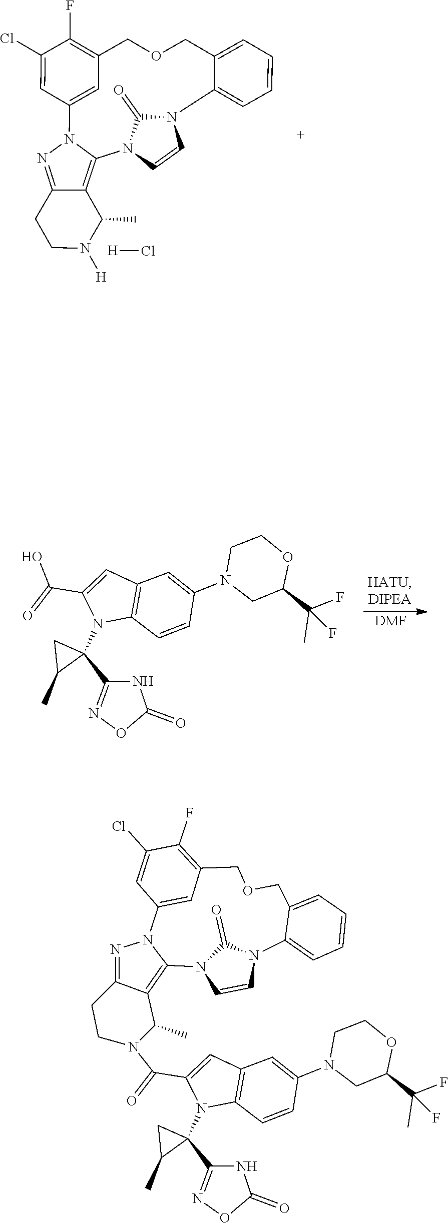

### Procedure for Synthesis of Compound 175

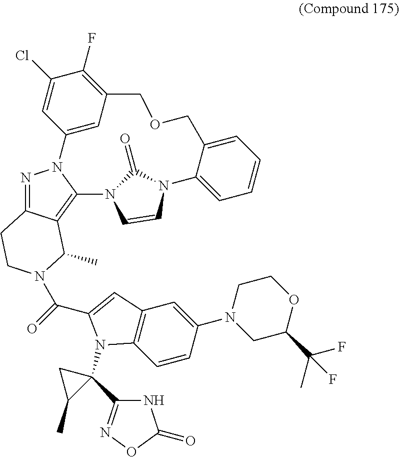

### (8S,10M)-26-chloro-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (8S,10M)-26-chloro-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (18.6 mg, 36.9 μmol) and 5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolecarboxylic acid (18.2 mg, 40.6 μmol) in DMF (400 μL) was added N-ethyldiisopropylamine (26 μL, 148 μmol) and HATU (21.1 mg, 55.4 μmol). The reaction stirred at room temperature overnight. The crude reaction was directly purified by Prep HPLC (Isco ACCQ Prep, Gemini column NX-C18, 250×21 mm, 20 mL/min, 45 minute separation, 20 to 80% H₂O/ACN with 0.1% Formic acid buffer), pure fractions were collected and lyophilized to give (8S,10M)-26-chloro-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one. m/z (ESI-pos) M+1=896.6; ¹H NMR (400 MHz, CHLOROFORM-D) δ 11.26 (d, J=23.8 Hz, 1H), 7.75 (ddd, J=14.2, 6.1, 2.8 Hz, 1H), 7.59-7.34 (m, 5H), 7.19-7.07 (m, 3H), 6.70 (d, J=32.7 Hz, 1H), 6.51-5.90 (m, 2H), 5.84-5.22 (m, 1H), 4.94-4.45 (m, 3H), 4.32 (dd, J=32.4, 9.2 Hz, 1H), 4.21-3.80 (m, 3H), 3.72-3.53 (m, 2H), 3.37 (d, J=12.0 Hz, 1H), 3.07 (dqd, J=31.7, 16.0, 5.1 Hz, 2H), 2.88 (td, J=11.8, 3.3 Hz, 1H), 2.78 (t, J=11.0 Hz, 1H), 1.99 (s, 1H), 1.87 (dt, J=22.5, 6.3 Hz, 1H), 1.76 (d, J=19.5 Hz, 1H), 1.71-1.62 (m, 4H), 1.60-1.47 (m, 1H), 1.40-1.22 (m, 2H), 1.16 (dd, J=26.6, 6.0 Hz, 3H).

### Example 176—Synthesis of Compound 176

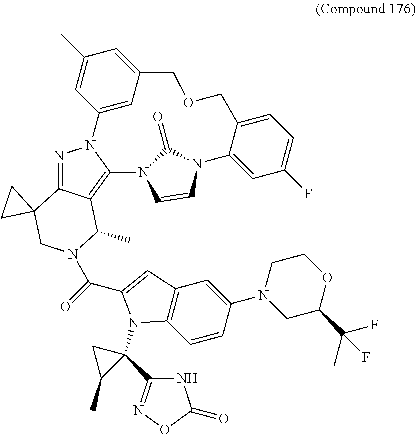

### (8S,10M)-17-fluoro-8,26-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)spiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one

Compound 176 was synthesized following the procedure of Example 168, with tert-butyl (2S)-3-cyano-2-methyl-4-oxo-1-piperidinecarboxylate being replaced with tert-butyl (6S)-7-cyano-6-methyl-8-oxo-5-aza-5-spiro[2.5]octanecarboxylate in step D. m/z (ESI-pos) M+1=906.6; ¹H NMR (400 MHz, CHLOROFORM-D) δ 11.30 (d, J=14.1 Hz, 1H), 7.65-7.34 (m, 3H), 7.30-7.03 (m, 4H), 7.01-6.88 (m, 2H), 6.72 (d, J=28.9 Hz, 1H), 6.46-5.89 (m, 2H), 5.93-5.25 (m, 1H), 4.55 (d, J=35.3 Hz, 2H), 4.31-3.99 (m, 6H), 3.95-3.87 (m, 1H), 3.81 (d, J=13.7 Hz, 1H), 3.65 (d, J=13.9 Hz, 1H), 3.54 (d, J=10.9 Hz, 1H), 3.36 (d, J=11.9 Hz, 1H), 3.09-2.81 (m, 2H), 2.38 (d, J=8.2 Hz, 3H), 1.92-1.79 (m, 2H), 1.68 (dd, J=15.3, 6.5 Hz, 3H), 1.48-1.27 (m, 1H), 1.17 (dd, J=21.1, 5.2 Hz, 3H), 1.08 (d, J=6.1 Hz, 1H), 0.84 (dt, J=10.5, 5.7 Hz, 1H).

### Example 177—Synthesis of Compound 177

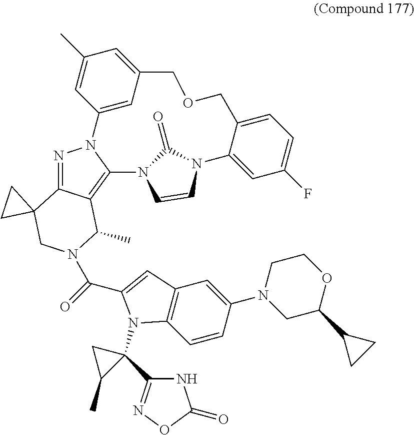

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8,26-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one

Compound 177 was synthesized following the procedure of Example 168, with tert-butyl (2S)-3-cyano-2-methyl-4-oxo-1-piperidinecarboxylate being replaced with tert-butyl (6S)-7-cyano-6-methyl-8-oxo-5-aza-5-spiro[2.5]octanecarboxylate in step D and 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid being replaced with 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid in step J. m/z (ESI-pos) M+1=878.6; ¹H NMR (400 MHz, CHLOROFORM-D) δ 11.32 (d, J=14.2 Hz, 1H), 7.51 (dq, J=23.8, 7.1 Hz, 2H), 7.38 (d, J=12.4 Hz, 1H), 7.23-7.05 (m, 4H), 7.02-6.89 (m, 2H), 6.72 (d, J=34.4 Hz, 1H), 6.46-6.18 (m, 2H), 5.93-5.33 (m, 1H), 4.54 (d, J=38.7 Hz, 2H), 4.26-4.12 (m, 1H), 4.08-4.00 (m, 3H), 3.79 (t, J=12.5 Hz, 1H), 3.67 (d, J=14.0 Hz, 1H), 3.52 (d, J=11.6 Hz, 1H), 3.34 (d, J=11.6 Hz, 1H), 2.99-2.82 (m, 1H), 2.73 (dd, J=13.5, 8.3 Hz, 1H), 2.38 (d, J=8.6 Hz, 3H), 1.93-1.79 (m, 2H), 1.68 (dd, J=14.8, 6.4 Hz, 3H), 1.57-1.48 (m, 2H), 1.36 (ddd, J=24.9, 9.4, 5.4 Hz, 2H), 1.25-1.05 (m, 4H), 0.95 (tt, J=8.3, 3.8 Hz, 1H), 0.84 (dt, J=11.5, 6.3 Hz, 1H), 0.59 (dddd, J=25.6, 13.0, 8.6, 4.6 Hz, 2H), 0.46 (dt, J=9.0, 4.5 Hz, 1H), 0.30 (dq, J=9.9, 4.9 Hz, 1H).

### Example 178—Synthesis of Compound 178

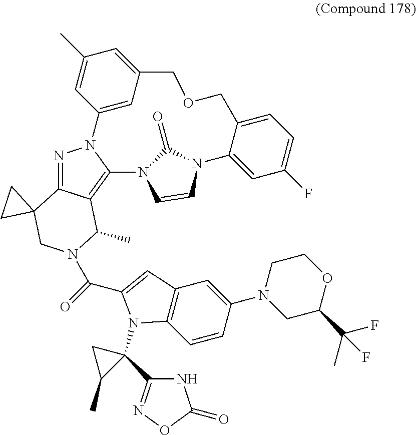

### (8S,10M)-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8,26-dimethylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one

Compound 178 synthesized following the procedure of Example 168, with tert-butyl(2S)-3-cyano-2-methyl-4-oxo-1-piperidinecarboxylate being replaced with tert-butyl (6S)-7-cyano-6-methyl-8-oxo-5-aza-5-spiro[2.5]octanecarboxylate in step D and 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid being replaced with 5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolecarboxylic acid in step J. m/z (ESI-pos) M+1=902.5; ¹H NMR (400 MHz, CHLOROFORM-D) δ 11.31 (d, J=14.3 Hz, 1H), 7.62-7.34 (m, 3H), 7.21-7.05 (m, 4H), 7.06-6.84 (m, 2H), 6.71 (d, J=28.1 Hz, 1H), 6.47-6.18 (m, 2H), 5.98-5.29 (m, 1H), 4.55 (d, J=34.2 Hz, 2H), 4.32-4.01 (m, 4H), 3.92-3.77 (m, 2H), 3.61 (dd, J=37.2, 12.8 Hz, 2H), 3.36 (d, J=11.9 Hz, 1H), 3.01-2.71 (m, 2H), 2.38 (d, J=8.3 Hz, 3H), 1.91-1.79 (m, 2H), 1.76-1.59 (m, 7H), 1.61-1.42 (m, 2H), 1.24-1.03 (m, 4H), 0.84 (dt, J=11.5, 5.8 Hz, 1H).

### Example 179—Synthesis of Compound 179

### Synthetic Scheme

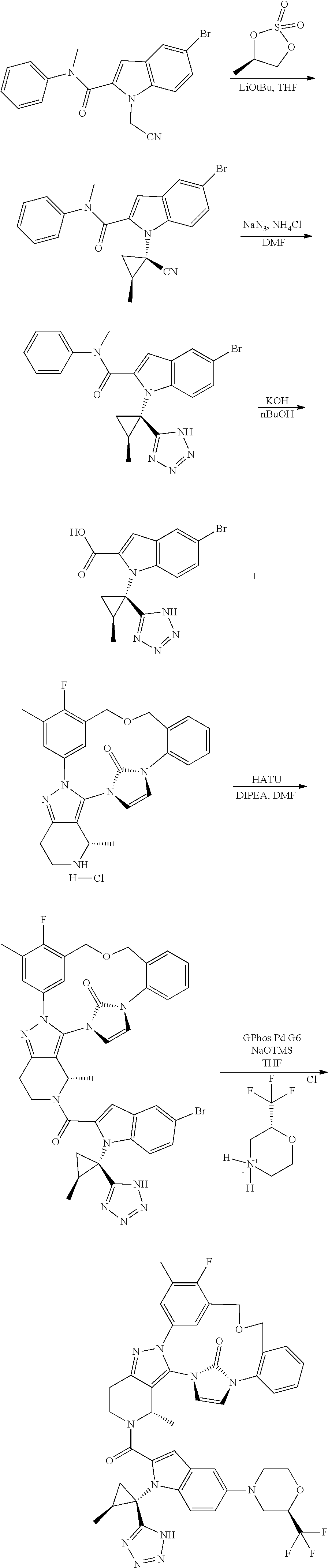

### Procedure for Synthesis of Compound 179

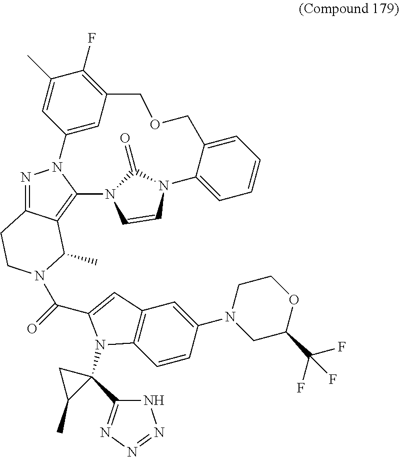

### (8S,10M)-25-fluoro-8,26-dimethyl-7-({1-[(1S,2S)-2-methyl-1-(1H-tetraazol-5-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Step A: N-methyl-N-phenyl-5-bromo-1-(cyanomethyl)-2-indolecarboxamide (6.81 g, 18.5 mmol), (R)-4-methyl-1,3,226-dioxathiolane-2,2-dione (5.11 g, 37 mmol), and THF (92.5 mL) were added to a flask and cooled to 0° C. Lithium 2-methyl-2-propanolate (10.4 g, 129 mmol) was added, and the mixture was stirred for 16 h, warming to room temperature. The mixture was quenched with addition of aqueous saturated NH₄Cl and the mixture was extracted twice with EtOAc. The combined organics were washed with brine, dried over sodium sulfate, filtered and concentrated. The crude residue was purified by column chromatography (0-30% EtOAc in heptane) to afford N-methyl-N-phenyl-5-bromo-1-[(1S,2S)-1-cyano-2-methylcyclopropyl]-2-indolecarboxamide (4.81 g, 11.8 mmol). m/z (ESI-pos) M+1=408.2/410.1

Step B: A vial was charged with N-methyl-N-phenyl-5-bromo-1-[(1S,2S)-1-cyano-2-methylcyclopropyl]-2-indolecarboxamide (150 mg, 367 μmol), sodium azide (119 mg, 1.84 mmol), and ammonium chloride (98.3 mg, 1.84 mmol) in DMF (750 μL) and the reaction was heated to 110° C. overnight. Upon completion, the reaction was cooled to room temperature, diluted with H₂O, and extracted with EtOAc. The organic layers were combined and washed with brine, dried over Na₂SO₄, filtered and concentrated. The crude material was purified by silica gel chromatography (0-50% EtOAc in heptane) to give N-methyl-N-phenyl-5-bromo-1-[(1S,2S)-2-methyl-1-(1H-tetraazol-5-yl)cyclopropyl]-2-indolecarboxamide (0.1 g, 222 μmol). m/z (ESI-pos) M+1=451.2/453.2

Step C: A solution of N-methyl-N-phenyl-5-bromo-1-[(1S,2S)-2-methyl-1-(1H-tetraazol-5-yl)cyclopropyl]-2-indolecarboxamide (0.1 g, 222 μmol) and KOH (173 mg, 3.08 mmol) in n-butanol (443 μL) was heated to 100° C. in a sealed vial for 2.5 h. The reaction was cooled to room temperature and then diluted with DCM and H₂O. 1M HCl was added and the aqueous phase was extracted with DCM. The combined organic phases were washed with brine, dried over Na₂SO₄, filtered, and concentrated. The crude material 5-bromo-1-[(1S,2S)-2-methyl-1-(1H-tetraazol-5-yl)cyclopropyl]-2-indolecarboxylic acid (73 mg, 202 μmol) was used in the following step without purification. m/z (ESI-pos) M+1=362.1/364.0

Step D: To a solution of (8S,10M)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (12.4 mg, 25.7 μmol) and 5-bromo-1-[(1S,2S)-2-methyl-1-(1H-tetraazol-5-yl)cyclopropyl]-2-indolecarboxylic acid (10.3 mg, 28.3 μmol) in DMF (200 μL) was added HATU (14.7 mg, 38.6 μmol) and N-ethyldiisopropylamine (17.9 μL, 103 μmol). The mixture stirred at 35° C. for 1 h. Upon completion, the reaction mixture was purified by Prep HPLC (Isco ACCQ Prep, Gemini column NX-C18, 250×21 mm, 20 mL/min, 45 minute separation, 20 to 80% H₂O/ACN with 0.1% Formic acid buffer), pure fractions were collected and lyophilized to give (8S,10M)-7-({5-bromo-1-[(1S,2S)-2-methyl-1-(1H-tetraazol-5-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (18.1 mg, 22.9 μmol). m/z (ESI-pos) M+1=789.4/791.4

Step E: A vial was charged with (8S,10M)-7-({5-bromo-1-[(1S,2S)-2-methyl-1-(1H-tetraazol-5-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (18 mg, 22.8 μmol), bromidopalladium(1+) 2-(trimethylsilyl)ethyl 1-benzenid-4-carboxylate-(3-tert-butoxy-2′,6′-diisopropyl-6-methoxy-2-biphenylyl)dicyclohexylphosphine (1/1) (1.08 mg, 1.14 μmol), (R)-2-(trifluoromethyl)morpholine-hydrogen chloride (1/1) (6.55 mg, 34.2 μmol), sodium trimethylsilanolate (7.67 mg, 68.4 μmol), followed by addition of THF (570 μL). and the reaction was sparged for 2 minutes then heated to 70° C. for 12 h. After 12 h, starting material remained so an additional portion of each reagent was added. The reaction was again sparged for 2 min then heated to 70° C. for an additional 3 hours. The reaction was cooled to room temperature, filtered through an HPLC filter, concentrated, and then purified by Prep HPLC (Isco ACCQ Prep, Gemini column NX-C18, 250×21 mm, 20 mL/min, 45-minute separation, 20 to 80% H₂O/ACN with 0.1% Formic acid buffer), pure fractions were collected and lyophilized to give (8S,10M)-25-fluoro-8,26-dimethyl-7-({1-[(1S,2S)-2-methyl-1-(1H-tetraazol-5-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (10.9 mg, 12.7 μmol). m/z (ESI-pos) M+1=864.5; ¹H NMR (400 MHz, CHLOROFORM-D) δ 7.70-7.29 (m, 5H), 7.16-6.97 (m, 3H), 6.76-6.62 (m, 1H), 6.46-5.88 (m, 2H), 5.89-5.23 (m, 1H), 5.02-4.63 (m, 2H), 4.52 (dd, J=13.8, 5.0 Hz, 1H), 4.27 (dd, J=29.2, 9.2 Hz, 1H), 4.20-4.09 (m, 3H), 3.88 (td, J=11.4, 2.7 Hz, 1H), 3.67 (td, J=13.1, 4.1 Hz, 1H), 3.52 (d, J=11.5 Hz, 1H), 3.32 (d, J=11.9 Hz, 1H), 3.23-2.99 (m, 2H), 2.95-2.78 (m, 2H), 2.39-2.28 (m, 3H), 2.17 (dt, J=21.7, 6.5 Hz, 1H), 1.84-1.72 (m, 2H), 1.72-1.31 (m, 3H), 1.24 (s, 1H), 0.90 (dd, J=32.3, 6.2 Hz, 3H).

### Example 180—Synthesis of Compound 180

### Synthetic Scheme

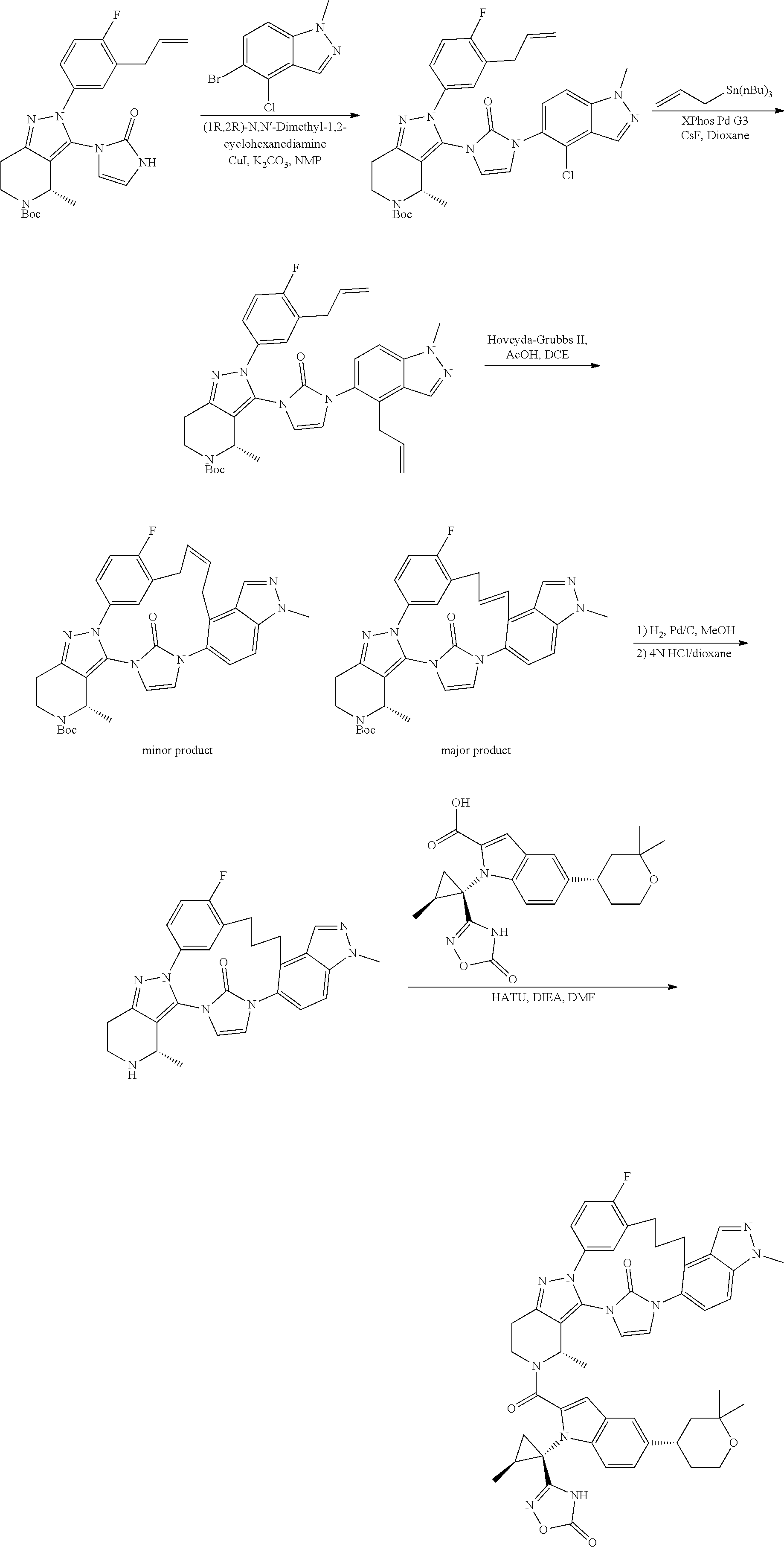

### Procedure for Synthesis of Compound 180

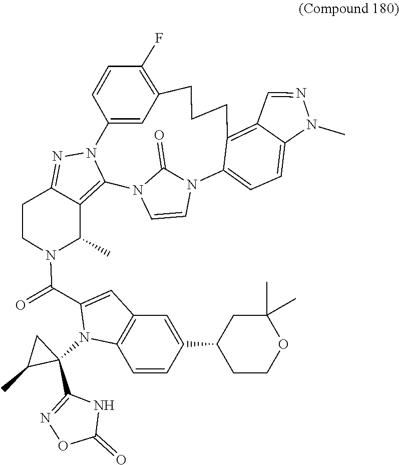

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-28-fluoro-8-methyl-19-methyl-2,3,7,11,14,19,20-heptaazaheptacyclo[25.3.1.1¹¹,¹⁴.0²,¹⁰.0⁴,⁹.0¹⁵,²³,0¹⁸,²²]dotriaconta-1(31),3,9,12,15,17,20,22,27,29-decaen-32-one

Step A: N-methyl-2-pyrrolidone (1.54 mL) was added to an 8 mL vial containing tert-butyl (S)-2-(3-allyl-4-fluorophenyl)-4-methyl-3-(2-oxo-2,3-dihydro-1H-imidazol-1-yl)-1,2,5-triaza-2,4,6,7-tetrahydro-5H-indene-5-carboxylate (140 mg, 309 μmol), 5-bromo-4-chloro-1-methyl-1H-indazole (152 mg, 618 μmol), dipotassium carbonate (128 mg, 926 μmol), copper iodide (29.4 mg, 154 μmol), and (1R,2R)-1,2-bis(methylamino)cyclohexane (49.3 μL, 309 μmol). The mixture was sparged with nitrogen for 5 minutes, then heated to 135° C. for 5 hours. The reaction mixture was diluted with ethyl acetate and 1:1 saturated aqueous NH₄Cl/brine. The organic layer was dried over anhydrous Na₂SO₄, filtered and the filtrate was evaporated under reduced pressure to give the crude compound and purified by over silica eluting with 20-90% EtOAc/Heptane to afford tert-butyl (S)-2-(3-allyl-4-fluorophenyl)-3-[3-(4-chloro-1-methyl-1H-indazol-5-yl)-2-oxo-2,3-dihydro-1H-imidazol-1-yl]-4-methyl-1,2,5-triaza-2,4,6,7-tetrahydro-5H-indene-5-carboxylate (110 mg, 178 μmol). m/z (ESI-pos) M+1=618.4

Step B: To a solution of tert-butyl (S)-2-(3-allyl-4-fluorophenyl)-3-[3-(4-chloro-1-methyl-1H-indazol-5-yl)-2-oxo-2,3-dihydro-1H-imidazol-1-yl]-4-methyl-1,2,5-triaza-2,4,6,7-tetrahydro-5H-indene-5-carboxylate (96.8 mg, 157 μmol), caesium fluoride (95.2 mg, 626 μmol), and XPhos Pd G3 (13.3 mg, 15.7 μmol) in 1,4-dioxane (1.57 mL) was added allyltris(butyl)stannane (146 μL, 470 μmol). The mixture was sparged with nitrogen for 5 minutes, then heated to 90° C. for 3 hours. The reaction was directly loaded onto a silica samplet and purified over silica eluting with 10-100% EtOAc/Heptane to afford tert-butyl (S)-2-(3-allyl-4-fluorophenyl)-3-[3-(4-allyl-1-methyl-1H-indazol-5-yl)-2-oxo-2,3-dihydro-1H-imidazol-1-yl]-4-methyl-1,2,5-triaza-2,4,6,7-tetrahydro-5H-indene-5-carboxylate (95.7 mg, 153 μmol). m/z (ESI-pos) M+1=624.4

Step C: To a 250 mL flask was added tert-butyl (S)-2-(3-allyl-4-fluorophenyl)-3-[3-(4-allyl-1-methyl-1H-indazol-5-yl)-2-oxo-2,3-dihydro-1H-imidazol-1-yl]-4-methyl-1,2,5-triaza-2,4,6,7-tetrahydro-5H-indene-5-carboxylate (95.7 mg, 153 μmol), 1,2-dichloroethane (100 mL), and acetic acid (17.6 μL, 307 μmol). The mixture was sparged with nitrogen for 10 minutes, then ruthenium 1,3-bis(mesityl)-2,2-imidazolidinediide [(4-isopropoxy-3-methanediidyltolylsulfonyl)-N-methylamino]methane dichloride (90.1 mg, 123 μmol) was added, and the reaction was heated to 60° C. while sparging with nitrogen for 4 hours. The reaction was concentrated in vacuo and purified via column chromatography eluting with 10-100% EtOAc/Heptane to remove the ruthenium catalyst. The mixture was then purified by ACCQ reverse-phase prep-HPLC purification eluting with 20-80% MeCN+0.1% formic acid in water+0.1% formic acid to afford tert-butyl (S)-8-methyl-19-methyl-32-oxo-2,3,7,11,14,19,20-heptaazaheptacyclo[25.3.1.1¹¹,¹⁴.0²,¹⁰.0⁴,⁹.0¹⁵,²³,0¹⁸,²²]dotriaconta-1(30),3,9,12,15 (23),16,18(22),20,24,27(31),28-undecaene-7-carboxylate (major product) (24 mg, 41.3 μmol) and tert-butyl (8S,25Z)-29-fluoro-8-methyl-19-methyl-33-oxo-2,3,7,11,14,19,20-heptaazaheptacyclo[26.3.1.1¹¹,¹⁴.0²,¹⁰.0⁴,⁹.0¹⁵,²³,0¹⁸,²²]tritriaconta-1(31),3,9,12,15,17,20,22,25,28 (32),29-undecaene-7-carboxylate (minor product) (8.3 mg, 13.9 μmol) as a mixture of alkene isomers. m/z (ESI-pos) M+1=582.4 and 596.4

Step D: Tert-butyl (S)-8-methyl-19-methyl-32-oxo-2,3,7,11,14,19,20-heptaazaheptacyclo[25.3.1.1¹¹,¹⁴.0²,¹⁰.0⁴,⁹.0¹⁵,²³,0¹⁸,²²]dotriaconta-1(30),3,9,12,15 (23),16,18(22),20,24,27(31),28-undecaene-7-carboxylate (major product) (23 mg, 39.5 μmol) was dissolved in methanol (2 mL), 10% palladium on carbon (40 mg) was added, and the mixture was sparged with hydrogen for one minute, then left stirring under hydrogen balloon atmosphere for 16 hours. The reaction was diluted with DCM, filtered, and concentrated in vacuo to afford tert-butyl (S)-28-fluoro-8-methyl-19-methyl-32-oxo-2,3,7,11,14,19,20-heptaazaheptacyclo[25.3.1.1¹¹,¹⁴.0²,¹⁰.0⁴,⁹.0¹⁵,²³,0¹⁸,²²]dotriaconta-1(30),3,9,12,15,17,20,22,27 (31),28-decaene-7-carboxylate (23.1 mg, 39.6 μmol). m/z (ESI-pos) M+1=584.4

Step E: 4M HCl in Dioxane (1 mL) was added to a stirring solution of tert-butyl (S)-28-fluoro-8-methyl-19-methyl-32-oxo-2,3,7,11,14,19,20-heptaazaheptacyclo[25.3.1.1¹¹,¹⁴.0²,¹⁰.0⁴,⁹.0¹⁵,²³,0¹⁸,²²]dotriaconta-1(30),3,9,12,15,17,20,22,27 (31),28-decaene-7-carboxylate (23.1 mg, 39.6 μmol) in dichloromethane (1 mL). The reaction was stirred at room temperature for 30 minutes, then concentrated in vacuo to afford (S)-28-fluoro-8-methyl-19-methyl-2,3,7,11,14,19,20-heptaazaheptacyclo[25.3.1.1¹¹,¹⁴.0²,¹⁰.0⁴,⁹.0¹⁵,²³,0¹⁸,²²]dotriaconta-1(30),3,9,12,15,17,20,22,27 (31),28-decaen-7-ium-32-one chloride (20.6 mg, 39.6 μmol). m/z (ESI-pos) M+1=484.4

Step F: To a solution of (S)-28-fluoro-8-methyl-19-methyl-2,3,7,11,14,19,20-heptaazaheptacyclo[25.3.1.1¹¹,¹⁴.0²,¹⁰.0⁴,⁹.0¹⁵,²³,0¹⁸,²²]dotriaconta-1(30),3,9,12,15,17,20,22,27 (31),28-decaen-7-ium-32-one chloride (20.6 mg, 39.6 μmol) in dimethylformamide (350 μL) was added N-ethyldiisopropylamine (27.6 μL, 158 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (32.6 mg, 79.2 μmol) followed by [(dimethylamino)(3-hydroxy-1λ⁵,2,3,4-tetraaza-3H-inden-1-yl)methylene]bis(methyl)azanium hexafluoridophosphate(1−) (31.7 mg, 83.2 μmol), and the mixture was stirred at room temperature for 16 hours. The reaction mixture was diluted with ethyl acetate and 1:1 saturated aqueous NH₄Cl/brine. The organic layer was dried over anhydrous Na₂SO₄, filtered and the filtrate was evaporated under reduced pressure. The crude residue was purified via column chromatography eluting with 20-100% EtOAc/Heptane to afford (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-28-fluoro-8-methyl-19-methyl-2,3,7,11,14,19,20-heptaazaheptacyclo[25.3.1.1¹¹,¹⁴.0²,¹⁰.0⁴,⁹.0¹⁵,²³,0¹⁸,²²]dotriaconta-1(30),3,9,12,15,17,20,22,27 (31),28-decaen-32-one (13 mg, 14.8 μmol). m/z (ESI-pos) M+1=877.6; ¹H NMR (400 MHz, CHLOROFORM-D) δ 11.34-11.24 (m, 1H), 8.14-7.99 (m, 1H), 7.63-7.12 (m, 7H), 6.81-6.67 (m, 1H), 6.29-6.02 (m, 2H), 5.92-5.76 (m, 1H), 5.31-4.41 (m, 1H), 4.17-4.04 (m, 3H), 3.92-3.78 (m, 2H), 3.70-3.54 (m, 1H), 3.22-2.96 (m, 4H), 2.84-2.65 (m, 2H), 2.49-2.12 (m, 3H), 1.96-1.48 (m, 10H), 1.42-1.19 (m, 10H).

### Example 181—Synthesis of Compound 181

### Procedure for Synthesis of Compound 181

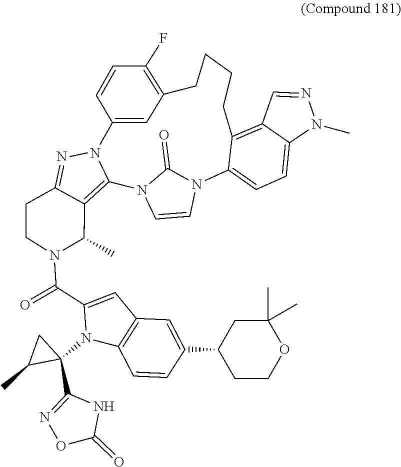

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-29-fluoro-8-methyl-19-methyl-2,3,7,11,14,19,20-heptaazaheptacyclo[26.3.1.1¹¹,¹⁴.0²,¹⁰.0⁴.⁹.0¹⁵,²³.0¹⁸,²²]tritriaconta-1(32),3,9,12,15,17,20,22,28,30-decaen-33-one

Compound 181 was synthesized following the procedure of Example 180, with tert-butyl (S)-8-methyl-19-methyl-32-oxo-2,3,7,11,14,19,20-heptaazaheptacyclo[25.3.1.1¹¹,¹⁴.0²,¹⁰.0⁴.⁹.0¹⁵,²³.0¹⁸,²²]dotriaconta-1(30),3,9,12,15 (23),16,18(22),20,24,27(31),28-undecaene-7-carboxylate (major product) being replaced with tert-butyl (S)-29-fluoro-8-methyl-19-methyl-33-oxo-2,3,7,11,14,19,20-heptaazaheptacyclo[26.3.1.1¹¹,¹⁴.0²,¹⁰.0⁴.⁹.0¹⁵,²³.0¹⁸,²²]tritriaconta-1(31),3,9,12,15,17,20,22,25,28 (32),29-undecaene-7-carboxylate (minor product). m/z (ESI-pos) M+1=891.6; ¹H NMR (400 MHz, CHLOROFORM-D) δ 11.27 (d, J=22.5 Hz, 1H), 8.11-8.00 (m, 1H), 7.65-7.10 (m, 7H), 6.71 (d, J=4.1 Hz, 1H), 6.49-6.14 (m, 2H), 5.98-5.74 (m, 1H), 5.32-4.43 (m, 1H), 4.09 (d, J=10.4 Hz, 3H), 3.87 (s, 2H), 3.69-3.59 (m, 1H), 3.45-2.55 (m, 8H), 1.95-1.17 (m, 23H).

### Example 182—Synthesis of Compound 182

### Synthetic Scheme

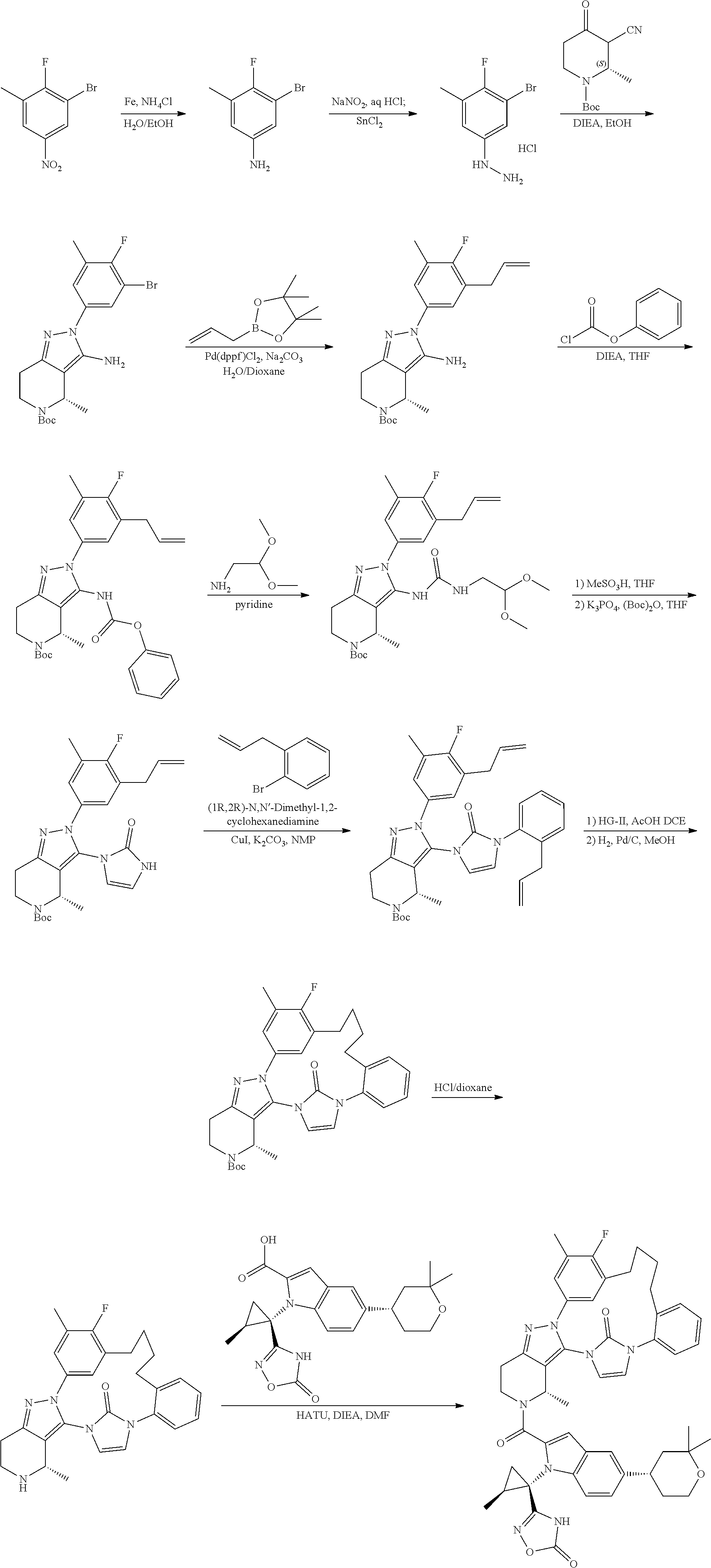

### Procedure for Synthesis of Compound 182

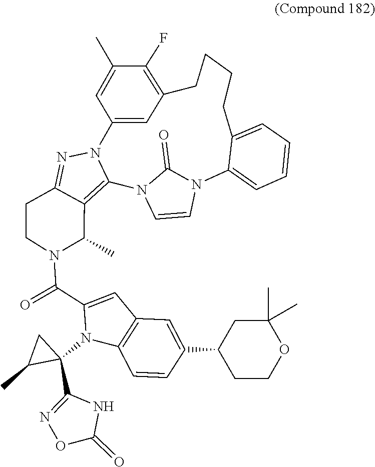

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8,27-dimethyl-2,3,7,11,14-pentaazahexacyclo[23.3.1.1¹¹,¹⁴.0²,¹⁰.0⁴.⁹.0¹⁵,²⁰]triaconta-1(29),3,9,12,15,17,19,25,27-nonaen-30-one

Step A: To the solution of 3-bromo-2-fluoro-5-nitrotoluene (1.00 g, 4.27 mmol) in ethanol (8.55 mL) was added water (2.14 mL), iron (1.19 g, 21.4 mmol) and ammonium chloride (2.29 mg, 42.7 mmol) under nitrogen. The reaction stirred at 70° C. for 24 hours. The reaction mixture was filtered through celite with EtOAc to remove the iron. The filtrate was diluted with water and extracted with ethyl acetate twice. The combined ethyl acetate layers were washed with brine, dried over Na₂SO₄, and concentrated in vacuo to afford 3-bromo-4-fluoro-5-tolylamine (826 mg, 4.05 mmol) which was used in the next step without purification. m/z (ESI-pos) M+1=204.0/206.0

Step B: Concentrated hydrochloric acid (10 mL) was added to 3-bromo-4-fluoro-5-tolylamine (826 mg, 4.05 mmol), and sodium nitrite (363 mg, 5.26 mmol) dissolved in water (2.5 mL) was added dropwise over 3 minutes while stirring vigorously at 0° C. and continued to stir for 30 minutes. A solution of dichloro-22-stannane-water (1/2) (1.92 g, 8.5 mmol) in water (2.5 mL) was then added over 2 minutes. Water (7.5 mL) was further added, and the mixture was stirred at room temperature for 45 minutes. The solid in the reaction mixture was collected by filtration over disposable filter funnel, washed with cold water (2×20 mL), and dried in a vacuum oven overnight to afford (3-bromo-4-fluoro-5-tolyl) hydrazine-hydrogen chloride (1/1) (595 mg, 2.33 mmol). m/z (ESI-pos) M+1=219.0/221.0

Step C: To a 40 mL vial was added tert-butyl(2S)-3-cyano-2-methyl-4-oxo-1-piperidinecarboxylate (555 mg, 2.33 mmol), 3-bromo-4-fluoro-5-toluidinoammonium chloride (595 mg, 2.33 mmol), EtOH (9.32 mL) and N-ethyldiisopropylamine (527 μL, 3.03 mmol). The reaction was capped and heated to 90° C. for 24 hours. The reaction was cooled and concentrated in vacuo. The residue was partitioned between EtOAc and water. Organics collected and aqueous layer extracted twice more with EtOAc. Combined organics were washed again with water, then brine, dried over sodium sulfate, filtered and concentrated in vacuo to afford tert-butyl (S)-3-amino-2-(3-bromo-4-fluoro-5-tolyl)-4-methyl-1,2,5-triaza-2,4,6,7-tetrahydro-5H-indene-5-carboxylate (1.02 g, 2.32 mmol) which was used in the next step without purification. m/z (ESI-pos) M+1=439.2/441.2

Step D: To a mixture of tert-butyl (S)-3-amino-2-(3-bromo-4-fluoro-5-tolyl)-4-methyl-1,2,5-triaza-2,4,6,7-tetrahydro-5H-indene-5-carboxylate (1.02 g, 2.32 mmol), disodium carbonate (492 mg, 4.64 mmol), water (2.32 mL), 1,4-dioxane (11.6 mL) was added Pd(dppf)Cl₂ (94.8 mg, 116 μmol) and 2-allyl-4,4,5,5-tetramethyl-1,3,2-dioxaborolane (1.31 mL, 6.97 mmol) at 25° C. The solution was purged with nitrogen for 10 minutes, vial was capped, and the resulting mixture was stirred at 80° C. for 3 hours. The reaction mixture was cooled down to room temperature and diluted with water. The aqueous mixture was extracted with ethyl acetate three times. The combined organic layers were washed with brine solution, dried over anhydrous Na₂SO₄, filtered and the filtrate was evaporated under reduced pressure to give the crude compound. The crude compound was purified by silica gel column chromatography eluting with 10-60% EtOAc/heptane to afford tert-butyl (S)-2-(3-allyl-4-fluoro-5-tolyl)-3-amino-4-methyl-1,2,5-triaza-2,4,6,7-tetrahydro-5H-indene-5-carboxylate (697 mg, 1.74 mmol). m/z (ESI-pos) M+1=401.3

Step E: To a solution of tert-butyl (S)-2-(3-allyl-4-fluoro-5-tolyl)-3-amino-4-methyl-1,2,5-triaza-2,4,6,7-tetrahydro-5H-indene-5-carboxylate (697 mg, 1.74 mmol) and N-ethyldiisopropylamine (909 μL, 5.22 mmol) in tetrahydrofuran (17.4 mL) was added benzenyl chloroformate (437 μL, 3.48 mmol), and the reaction was stirred at 25° C. for 30 minutes. The reaction was concentrated in vacuo to afford tert-butyl (S)-2-(3-allyl-4-fluoro-5-tolyl)-4-methyl-3-[phenyl(oxycarbonylamino)]-1,2,5-triaza-2,4,6,7-tetrahydro-5H-indene-5-carboxylate (906 mg, 1.74 mmol) and used directly in the next step. m/z (ESI-pos) M+1=521.4

Step F: Crude tert-butyl (S)-2-(3-allyl-4-fluoro-5-tolyl)-4-methyl-3-[phenyl(oxycarbonylamino)]-1,2,5-triaza-2,4,6,7-tetrahydro-5H-indene-5-carboxylate (906 mg, 1.74 mmol) was dissolved in pyridine (8.7 mL), to which 2,2-dimethoxyethylamine (569 μL, 5.22 mmol) was added. The mixture was stirred at room temperature for 30 minutes. The reaction mixture was concentrated to give the crude tert-butyl (S)-2-(3-allyl-4-fluoro-5-tolyl)-3-[3-(2,2-dimethoxyethyl)ureido]-4-methyl-1,2,5-triaza-2,4,6,7-tetrahydro-5H-indene-5-carboxylate (925 mg, 1.74 mmol) which was directly used for the next step. m/z (ESI-pos) M+1=532.4

Step G: A solution of crude tert-butyl (S)-2-(3-allyl-4-fluoro-5-tolyl)-3-[3-(2,2-dimethoxyethyl)ureido]-4-methyl-1,2,5-triaza-2,4,6,7-tetrahydro-5H-indene-5-carboxylate (925 mg, 1.74 mmol) in tetrahydrofuran (17.4 mL) was treated with methanesulfonic acid (565 μL, 8.7 mmol). The mixture was stirred at 60° C. for 30 minutes. Aqueous K₃PO₄ (0.5 M, ~4 mL) was added to adjusted pH to about 9, and then tert-butoxycarbonyl-tert-butyl carbonate (190 mg, 870 μmol) was added, and the resulting mixture was stirred at room temperature for 30 minutes. The reaction mixture was diluted with ethyl acetate and washed with water twice. The organic layer was then dried over anhydrous Na₂SO₄, filtered and the filtrate was evaporated under reduced pressure. The crude residue was purified via column chromatography eluting with 10-100% EtOAc/Heptane to afford tert-butyl 3-[1-(3-allyl-4-fluorophenyl)-5-(2-oxo-2,3-dihydro-1H-imidazol-1-yl)-4-pyrazolyl]-1-azetidinecarboxylate (285 mg, 648 μmol). m/z (ESI-pos) M+1=468.4

Step H: To a solution of tert-butyl (S)-2-(3-allyl-4-fluoro-5-tolyl)-4-methyl-3-(2-oxo-2,3-dihydro-1H-imidazol-1-yl)-1,2,5-triaza-2,4,6,7-tetrahydro-5H-indene-5-carboxylate (106 mg, 227 μmol) and o-allylbromobenzene (134 mg, 680 μmol) in N-methyl-2-pyrrolidone (1.13 mL) were added (1R,2R)-1,2-bis(methylamino)cyclohexane (36.2 μL, 227 μmol), dipotassium carbonate (125 mg, 907 μmol), and copper iodide (21.6 mg, 113 μmol). The reaction mixture was sparged with nitrogen for 5 minutes, then sealed with parafilm and heated at 130° C. for 3 hours. The reaction mixture was cooled to ambient temperature and diluted with ethyl acetate and washed with aqueous saturated ammonium chloride solution twice. The organic layer was dried over anhydrous Na₂SO₄ and evaporated under reduced pressure. The crude residue was purified via column chromatography eluting with 10-85% EtOAc/Heptane to afford tert-butyl (S)-2-(3-allyl-4-fluoro-5-tolyl)-3-[3-(o-allylphenyl)-2-oxo-2,3-dihydro-1H-imidazol-1-yl]-4-methyl-1,2,5-triaza-2,4,6,7-tetrahydro-5H-indene-5-carboxylate (80 mg, 137 μmol). m/z (ESI-pos) M+1=584.4

Step I: To a 250 mL flask was added tert-butyl (S)-2-(3-allyl-4-fluoro-5-tolyl)-3-[3-(o-allylphenyl)-2-oxo-2,3-dihydro-1H-imidazol-1-yl]-4-methyl-1,2,5-triaza-2,4,6,7-tetrahydro-5H-indene-5-carboxylate (80 mg, 137 μmol), 1,2-dichloroethane (80 mL), and acetic acid (7.84 μL, 137 μmol). The mixture was sparged with nitrogen for 10 minutes, then ruthenium 1,3-bis(mesityl)-2,2-imidazolidinediide [(4-isopropoxy-3-methanediidyltolylsulfonyl)-N-methylamino]methane dichloride (25.1 mg, 34.3 μmol) was added, and the reaction was heated to 40° C. in a closed system for 24 hours. The reaction was concentrated in vacuo and purified via column chromatography eluting with 10-90% EtOAc/Heptane followed by ACCQ reverse phase purification eluting with 35-75% MeCN+0.1% formic acid in water+0.1% formic acid. Desired fraction lyophilized overnight to afford tert-butyl (8S,22Z)-26-fluoro-8,27-dimethyl-30-oxo-2,3,7,11,14-pentaazahexacyclo[23.3.1.1¹¹,¹⁴.0²,¹⁰.0⁴.⁹.0¹⁵,²⁰]triaconta-1(28),3,9,12,15,17,19,22,25(29),26-decaene-7-carboxylate (44 mg, 79.2 μmol) as a mixture of alkene isomers. m/z (ESI-pos) M+1=556.4

Step J: Tert-butyl (8S,22Z)-26-fluoro-8,27-dimethyl-30-oxo-2,3,7,11,14-pentaazahexacyclo[23.3.1.1¹¹,¹⁴.0²,¹⁰.0⁴.⁹.0¹⁵,²⁰]triaconta-1(28),3,9,12,15,17,19,22,25(29),26-decaene-7-carboxylate (44 mg, 79.2 μmol) was dissolved in methanol (1 mL), 10% palladium on carbon (20 mg) was added, and the mixture was sparged with hydrogen for one minute, then left stirring under hydrogen balloon atmosphere for 1.5 hours. The reaction was diluted with DCM, filtered, and concentrated in vacuo. The crude residue was purified by ACCQ prep-HPLC purification eluting with 40-75% MeCN+0.1% formic acid in water+0.1% formic acid. Desired fractions were lyophilized overnight to afford tert-butyl (S)-26-fluoro-8,27-dimethyl-30-oxo-2,3,7,11,14-pentaazahexacyclo[23.3.1.1¹¹,¹⁴.0²,¹⁰.0⁴.⁹.0¹⁵,²⁰]triaconta-1(28),3,9,12,15,17,19,25(29),26-nonaene-7-carboxylate (26 mg, 46.6 μmol). m/z (ESI-pos) M+1=558.4

Step K: 4M HCl in dioxane (0.5 mL) was added to a stirring solution of tert-butyl (S)-26-fluoro-8,27-dimethyl-30-oxo-2,3,7,11,14-pentaazahexacyclo[23.3.1.1¹¹,¹⁴.0²,¹⁰.0⁴.⁹.0¹⁵,²⁰]triaconta-1(28),3,9,12,15,17,19,25(29),26-nonaene-7-carboxylate (26 mg, 46.6 μmol) in dichloromethane (0.5 mL). After 30 minutes, the reaction was concentrated in vacuo to afford (S)-26-fluoro-8,27-dimethyl-2,3,7,11,14-pentaazahexacyclo[23.3.1.1¹¹,¹⁴.0²,¹⁰.0⁴.⁹.0¹⁵,²⁰]triaconta-1(28),3,9,12,15,17,19,25(29),26-nonaen-7-ium-30-one chloride (23 mg, 46.6 μmol). m/z (ESI-pos) M+1=458.3

Step L: To a solution of (S)-26-fluoro-8,27-dimethyl-2,3,7,11,14-pentaazahexacyclo[23.3.1.1¹¹,¹⁴.0²,¹⁰.0⁴.⁹.0¹⁵,²⁰]triaconta-1(28),3,9,12,15,17,19,25(29),26-nonaen-7-ium-30-one chloride (23 mg, 46.6 μmol) in dimethylformamide (1 mL) was added N-ethyldiisopropylamine (40.5 μL, 233 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (38.3 mg, 93.1 μmol) followed by [(dimethylamino)(3-hydroxy-1λ⁵,2,3,4-tetraaza-3H-inden-1-yl)methylene]bis(methyl)azanium hexafluoridophosphate (1-) (37.3 mg, 97.8 μmol) and the mixture stirred at room temperature for 16 hours. The reaction mixture was directly purified by ACCQ reverse phase prep-HPLC eluting with 40-100% MeCN+0.1% formic acid in water+0.1% formic acid. Desired fractions were lyophilized overnight to afford (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8,27-dimethyl-2,3,7,11,14-pentaazahexacyclo[23.3.1.1¹¹,¹⁴.0²,¹⁰.0⁴.⁹.0¹⁵,²⁰]triaconta-1(29),3,9,12,15,17,19,25,27-nonaen-30-one (20 mg, 23.5 μmol). m/z (ESI-pos) M+1=851.6; ¹H NMR (400 MHz, CHLOROFORM-D) δ 11.29 (s, 1H), 7.63-7.45 (m, 2H), 7.40-7.13 (m, 6H), 7.11-6.91 (m, 1H), 6.79-6.67 (m, 1H), 6.44-5.88 (m, 2H), 5.89-5.17 (m, 1H), 4.94-4.41 (m, 1H), 3.93-2.77 (m, 8H), 2.73-2.35 (m, 2H), 2.34-2.27 (m, 3H), 1.94-1.01 (m, 25H).

### Example 183—Synthesis of Compound 183

### Synthetic Scheme

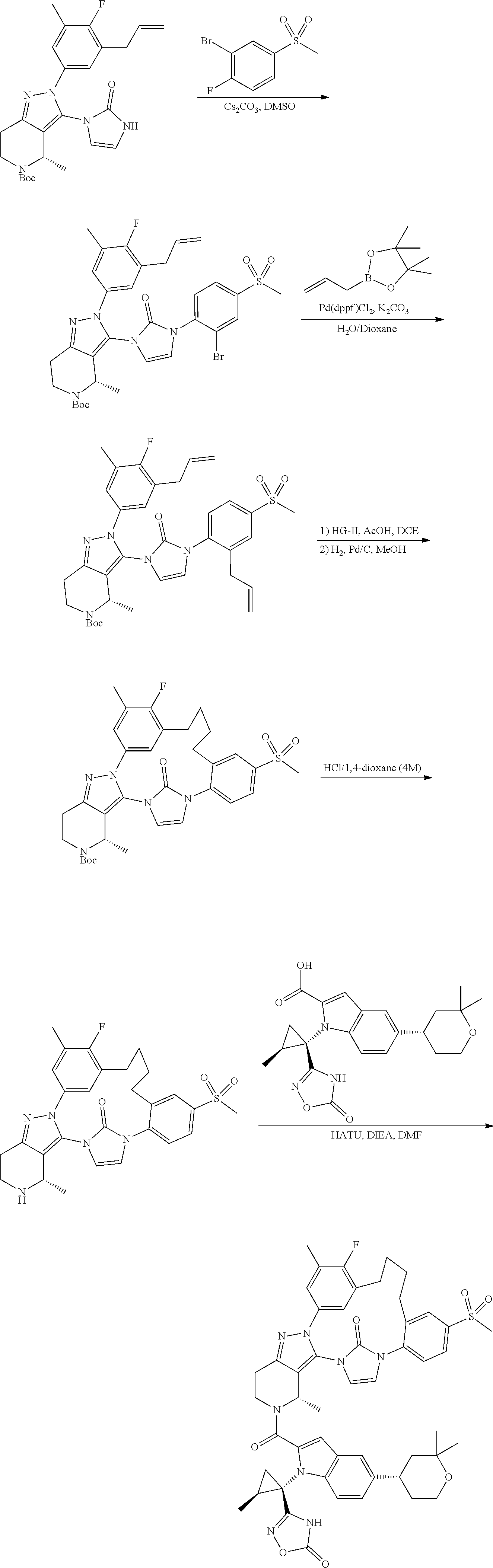

### Procedure for Synthesis of Compound 183

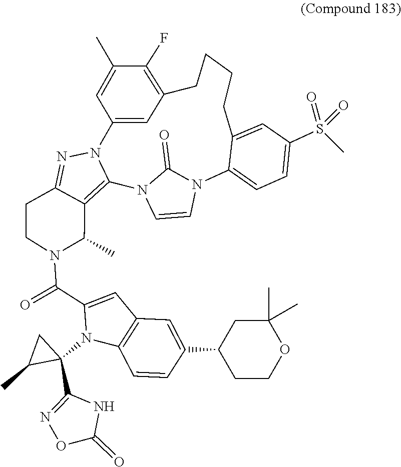 [SMILES: Cc1cc2cc(c1F)CCCCc1cc(S(C)(=O)=O)ccc1-n1ccn(c1=O)-c1c3c(nn1-2)CCN(C(=O)c1cc2cc([C@H]4CCOC(C)(C)C4)ccc2n1[C@@]1(c2noc(=O)[nH]2)C[C@@H]1C)[C@H]3C]

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-18-mesyl-8,27-dimethyl-2,3,7,11,14-pentaazahexacyclo[23.3.1.1¹¹,¹⁴.0²,¹⁰.0⁴.⁹.0¹⁵,²⁰]triaconta-1(29),3,9,12,15,17,19,25,27-nonaen-30-one

Step A: An 8 mL vial was charged with tert-butyl (S)-2-(3-allyl-4-fluoro-5-tolyl)-4-methyl-3-(2-oxo-2,3-dihydro-1H-imidazol-1-yl)-1,2,5-triaza-2,4,6,7-tetrahydro-5H-indene-5-carboxylate (95 mg, 203 μmol), 2-bromo-1-fluoro-4-mesylbenzene (154 mg, 610 μmol), dicaesium carbonate (132 mg, 406 μmol), and dimethyl sulfoxide (1.35 mL). The vial was capped and stirred at room temperature for 30 minutes. The reaction was then heated to 40° C. for 45 minutes. Reaction cooled and partitioned between water and extracted with EtOAc twice. Organics dried over brine, sodium sulfate, filtered and concentrated. Crude residue was purified via column chromatography eluting with 10-100% EtOAc/Heptane to afford tert-butyl (S)-2-(3-allyl-4-fluoro-5-tolyl)-3-[3-(2-bromo-4-mesylphenyl)-2-oxo-2,3-dihydro-1H-imidazol-1-yl]-4-methyl-1,2,5-triaza-2,4,6,7-tetrahydro-5H-indene-5-carboxylate (87 mg, 124 μmol). m/z (ESI-pos) M+1=700.3/702.4

Step B: Tert-butyl (S)-2-(3-allyl-4-fluoro-5-tolyl)-3-[3-(2-bromo-4-mesylphenyl)-2-oxo-2,3-dihydro-1H-imidazol-1-yl]-4-methyl-1,2,5-triaza-2,4,6,7-tetrahydro-5H-indene-5-carboxylate (87 mg, 124 μmol), Pd(dppf)Cl₂ (30.4 mg, 37.3 μmol), and dipotassium carbonate (85.8 mg, 621 μmol) were dissolved in 1,4-dioxane (1.24 mL) and water (207 μL), and 2-allyl-4,4,5,5-tetramethyl-1,3,2-dioxaborolane (116 μL, 621 μmol) was added. The resulting solution was degassed with nitrogen for 5 minutes, then heated to 90° C. for 2 hours. The reaction mixture was cooled down to room temperature and diluted with water. The aqueous mixture was extracted with ethyl acetate three times. The combined organic layers were washed with brine solution, dried over anhydrous Na₂SO₄, filtered, and the filtrate was evaporated under reduced pressure. The crude residue was purified by silica gel column chromatography eluting with 5-95% EtOAc/Heptane to afford tert-butyl (S)-2-(3-allyl-4-fluoro-5-tolyl)-3-[3-(2-allyl-4-mesylphenyl)-2-oxo-2,3-dihydro-1H-imidazol-1-yl]-4-methyl-1,2,5-triaza-2,4,6,7-tetrahydro-5H-indene-5-carboxylate (63.8 mg, 96.4 μmol). m/z (ESI-pos) M+1=662.4

Step C: To a 250 mL flask was added tert-butyl (S)-2-(3-allyl-4-fluoro-5-tolyl)-3-[3-(2-allyl-4-mesylphenyl)-2-oxo-2,3-dihydro-1H-imidazol-1-yl]-4-methyl-1,2,5-triaza-2,4,6,7-tetrahydro-5H-indene-5-carboxylate (63.8 mg, 96.4 μmol), 1,2-dichloroethane (65 mL), and acetic acid (5.51 μL, 96.4 μmol). The mixture was sparged with nitrogen for 10 minutes, then ruthenium o-isopropoxymethanediidyltoluene 1,3-bis(mesityl)-2,2-imidazolidinediide dichloride (15.1 mg, 24.1 μmol) was added and the reaction was heated to 50° C. for 16 hours with continuous sparging. The reaction was concentrated in vacuo and purified via column chromatography eluting with 10-90% EtOAc/Heptane to afford tert-butyl (S)-26-fluoro-18-mesyl-8,27-dimethyl-30-oxo-2,3,7,11,14-pentaazahexacyclo[23.3.1.1¹¹,¹⁴.0²,¹⁰.0⁴.⁹.0¹⁵,²⁰]triaconta-1(29),3,9,12,15(20),16,18,22,25,27-decaene-7-carboxylate (61.1 mg, 96.4 μmol) as a mixture of alkene isomers. m/z (ESI-pos) M+1=634.4

Step D: Tert-butyl (S)-26-fluoro-18-mesyl-8,27-dimethyl-30-oxo-2,3,7,11,14-pentaazahexacyclo[23.3.1.1¹¹,¹⁴.0²,¹⁰.0⁴.⁹.0¹⁵,²⁰]triaconta-1(29),3,9,12,15(20),16,18,22,25,27-decaene-7-carboxylate (61.1 mg, 96.4 μmol) was dissolved in methanol (2 mL), 10% palladium on carbon (30 mg) was added, and the mixture was sparged with hydrogen for one minute, then left stirring under hydrogen balloon atmosphere for 2 hours. The reaction was diluted with DCM, filtered, and concentrated in vacuo to afford tert-butyl (S)-26-fluoro-18-mesyl-8,27-dimethyl-30-oxo-2,3,7,11,14-pentaazahexacyclo[23.3.1.1¹¹,¹⁴.0²,¹⁰.0⁴.⁹.0¹⁵,²⁰]triaconta-1(29),3,9,12,15(20),16,18,25,27-nonaene-7-carboxylate (61.3 mg, 96.4 μmol). m/z (ESI-pos) M+1=636.4

Step E: 4N HCl in dioxane (1 mL) was added to a stirring solution of tert-butyl (S)-26-fluoro-18-mesyl-8,27-dimethyl-30-oxo-2,3,7,11,14-pentaazahexacyclo[23.3.1.1¹¹,¹⁴.0²,¹⁰.0⁴.⁹.0¹⁵,²⁰]triaconta-1(29),3,9,12,15(20),16,18,25,27-nonaene-7-carboxylate (61.3 mg, 96.4 μmol) in dichloromethane (2 mL). After 30 minutes, the reaction was concentrated in vacuo to afford (S)-26-fluoro-18-mesyl-8,27-dimethyl-2,3,7,11,14-pentaazahexacyclo[23.3.1.1¹¹,¹⁴.0²,¹⁰.0⁴.⁹.0¹⁵,²⁰]triaconta-1(29),3,9,12,15(20),16,18,25,27-nonaen-7-ium-30-one chloride (55.2 mg, 96.5 μmol). m/z (ESI-pos) M+1=536.4

Step F: An 8 mL vial was charged with(S)-26-fluoro-18-mesyl-8,27-dimethyl-2,3,7,11,14-pentaazahexacyclo[23.3.1.1¹¹,¹⁴.0²,¹⁰.0⁴.⁹.0¹⁵,²⁰]triaconta-1(29),3,9,12,15(20),16,18,25,27-nonaen-7-ium-30-one chloride (55.2 mg, 96.5 μmol), dimethylformamide (1 mL), and N-ethyldiisopropylamine (84 μL, 482 μmol). 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (79.4 mg, 193 μmol) was then added followed by [(dimethylamino)(3-hydroxy-1λ⁵,2,3,4-tetraaza-3H-inden-1-yl)methylene]bis(methyl)azanium hexafluoridophosphate (1-) (77.2 mg, 203 μmol) and the mixture was stirred at room temperature for 16 hours. Reaction was transferred with MeCN/H₂O and purified directly by reverse phase prep-HPLC purification eluting with 40-90% MeCN+0.1% formic acid in H₂O+0.1% formic acid. Desired fractions were dried in vacuo to afford (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-18-mesyl-8,27-dimethyl-2,3,7,11,14-pentaazahexacyclo[23.3.1.1¹¹,¹⁴.0²,¹⁰.0⁴.⁹.0¹⁵,²⁰]triaconta-1(29),3,9,12,15,17,19,25,27-nonaen-30-one (47.9 mg, 51.6 μmol). m/z (ESI-pos) M+1=929.6; ¹H NMR (400 MHz, CHLOROFORM-D) δ 11.30-11.20 (m, 1H), 7.95-7.75 (m, 2H), 7.62-7.45 (m, 3H), 7.43-7.17 (m, 2H), 7.09-6.88 (m, 1H), 6.77-6.66 (m, 1H), 6.50-5.97 (m, 2H), 5.87-5.15 (m, 1H), 4.94-4.42 (m, 1H), 3.92-3.51 (m, 3H), 3.43-2.78 (m, 8H), 2.69-2.39 (m, 2H), 2.34-2.26 (m, 3H), 1.94-0.98 (m, 23H).

### Example 184—Synthesis of Compound 184

### Synthetic Scheme

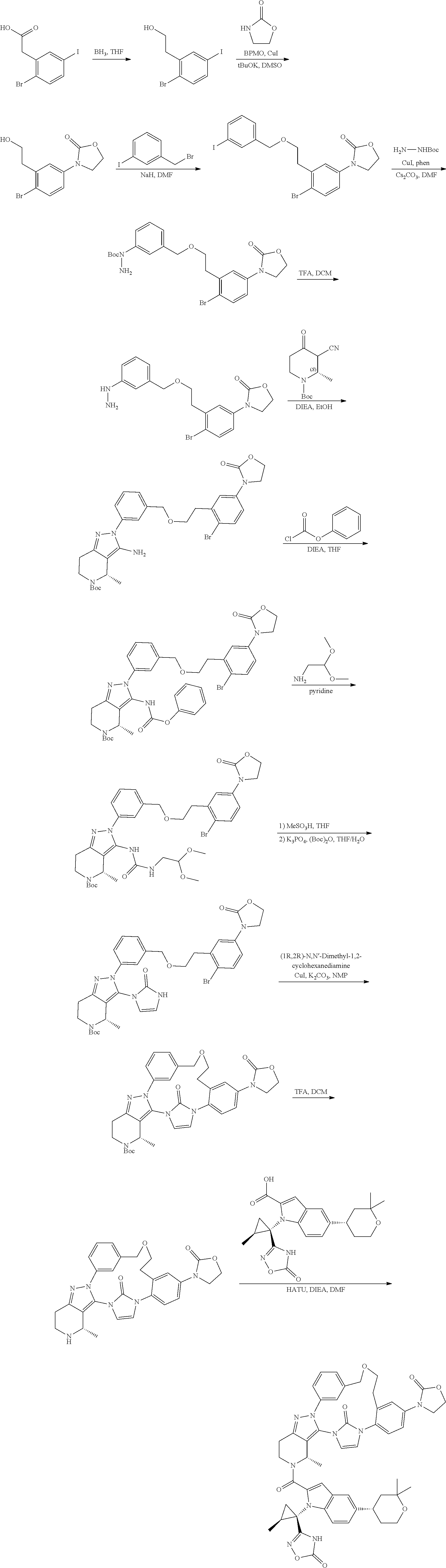

### Procedure for Synthesis of Compound 184

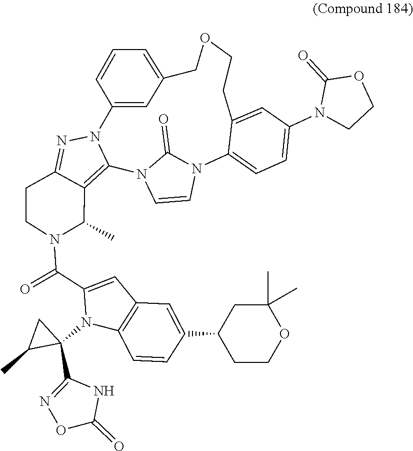 [SMILES: C[C@H]1c2c(nn3c2-n2ccn(c2=O)-c2ccc(N4CCOC4=O)cc2CCOCc2cccc-3c2)CCN1C(=O)c1cc2cc([C@H]3CCOC(C)(C)C3)ccc2n1[C@@]1(c2noc(=O)[nH]2)C[C@@H]1C]

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-18-(2-oxo-1,3-oxazolidin-3-yl)-23-oxa-2,3,7,11,14-pentaazahexacyclo[23.3.1.1¹¹,¹⁴.0²,¹⁰.0⁴.⁹.0¹⁵,²⁰]triaconta-1(29),3,9,12,15,17,19,25,27-nonaen-30-one

Step A: 1M Borane in THF (945 mg, 11 mmol) was added dropwise to a stirred solution of (2-bromo-5-iodophenyl) acetic acid (1.5 g, 4.4 mmol) in tetrahydrofuran (30 mL) cooled to 0° C. The resulting mixture was warmed to room temperature and stirred for 4 days. The mixture was poured into aqueous ammonium chloride solution and extracted with ethyl acetate. The combined organic phases were washed with brine, dried with sodium sulfate, and concentrated to afford 2-(2-bromo-5-iodophenyl) ethanol (1.43 g, 4.36 mmol) which was used in the next step without purification.

Step B: An 8 mL vial was charged with 2-(2-bromo-5-iodophenyl) ethanol (1.43 g, 4.36 mmol), 1,3-oxazolidin-2-one (570 mg, 6.55 mmol), 2-({[(2-pyridyl)methyl]carbamoylcarbonylamino}methyl)pyridine (118 mg, 436 μmol), copper iodide (83.1 mg, 436 μmol), and potassium 2-methyl-2-propanolate (735 mg, 6.55 mmol), followed by addition of dimethyl sulfoxide (4.36 mL). The solution got warm upon addition of DMSO. Reaction was sparged for 5 minutes with nitrogen, then stirred at room temperature for 20 hours. The reaction was diluted with EtOAc and washed with water, then brine. Organics dried over sodium sulfate, filtered, and concentrated in vacuo. The crude residue was purified via column chromatography eluting with 10-100% EtOAc/Heptane to afford 3-[4-bromo-3-(2-hydroxyethyl)phenyl]-1,3-oxazolidin-2-one (660 mg, 2.31 mmol). m/z (ESI-pos) M+1=286.1/288.1

Step C: To the solution of 3-[4-bromo-3-(2-hydroxyethyl)phenyl]-1,3-oxazolidin-2-one (660 mg, 2.31 mmol) in dimethylformamide (15.4 mL) was added 60% sodium hydride in mineral oil (185 mg, 4.61 mmol) at 0° C. under nitrogen. The solution was stirred at 0° C. for 30 minutes. m-(bromomethyl) iodobenzene (890 mg, 3 mmol) was then added and the reaction was stirred at 0° C. for 1 hour and then at room temperature for 1.5 hour. The reaction mixture was quenched with saturated aqueous NaHCO₃, diluted with ethyl acetate, and washed with brine twice. The organic layer was dried over Na₂SO₄ and concentrated in vacuo. The crude product was purified via column chromatography eluting with 5-75% EtOAc/Heptane to afford 3-(4-bromo-3-{2-[(m-iodophenyl)methoxy]ethyl}phenyl)-1,3-oxazolidin-2-one (1.07 g, 2.14 mmol). m/z (ESI-pos) M+1=502.1/504.1

Step D: A solution of 3-(4-bromo-3-{2-[(m-iodophenyl)methoxy]ethyl}phenyl)-1,3-oxazolidin-2-one (1.07 g, 2.14 mmol), tert-butyl carbazate (340 mg, 2.57 mmol), dicaesium carbonate (977 mg, 3 mmol), copper iodide (20.4 mg, 107 μmol), and 4,5-diazaphenanthrene (77.2 mg, 428 μmol) in dimethylformamide (2.14 mL) was sparged with nitrogen for 5 minutes and heated to 80° C. for 16 hours. The solution was taken up in EtOAc, filtered, and washed with brine three times. The organics were dried over Na₂SO₄, filtered, and concentrated. The crude product purified by silica gel chromatography eluting with 10-100% EtOAc/heptanes to afford 3-[4-bromo-3-(2-{[m-(tert-butyl-2-carboxyhydrazino)phenyl]methoxy}ethyl)phenyl]-1,3-oxazolidin-2-one (680 mg, 1.34 mmol). m/z (ESI-pos) M+1=406.2/408.2 (-boc)

Step E: Trifluoroacetic acid (2.06 mL, 26.9 mmol) was added to a solution of 3-[4-bromo-3-(2-{[m-(tert-butyl-2-carboxyhydrazino)phenyl]methoxy}ethyl)phenyl]-1,3-oxazolidin-2-one (680 mg, 1.34 mmol) in dichloromethane (5 mL). The mixture was stirred at room temperature for 1.5 hours. The mixture was quenched with addition of 2M aqueous Na₂CO₃ and the aqueous was extracted with DCM. Organics washed again with 2M aqueous Na₂CO3 solution, dried over magnesium sulfate, filtered and concentrated in vacuo to afford [m-({2-[2-bromo-5-(2-oxo-1,3-oxazolidin-3-yl)phenyl]ethoxy}methyl)phenyl]hydrazine (542 mg, 1.33 mmol). m/z (ESI-pos) M+1=406.1/408.1

Step F: To a 40 mL vial was added tert-butyl(2S)-3-cyano-2-methyl-4-oxo-1-piperidinecarboxylate (319 mg, 1.34 mmol), [m-({2-[2-bromo-5-(2-oxo-1,3-oxazolidin-3-yl)phenyl]ethoxy}methyl)phenyl]hydrazine (544 mg, 1.34 mmol), N-ethyldiisopropylamine (350 μL, 2.01 mmol), and ethanol (6.69 mL). The reaction was capped and heated to 80° C. for 16 hours. The reaction was cooled and concentrated in vacuo. The residue was then partitioned between EtOAc and water. Organics collected and aqueous layer extracted twice more with EtOAc. Combined organics were washed with brine, dried over sodium sulfate, filtered and concentrated in vacuo. Crude residue purified via column chromatography eluting with 5-100% EtOAc/Heptane to afford tert-butyl (S)-3-amino-2-[m-({2-[2-bromo-5-(2-oxo-1,3-oxazolidin-3-yl)phenyl]ethoxy}methyl)phenyl]-4-methyl-1,2,5-triaza-2,4,6,7-tetrahydro-5H-indene-5-carboxylate (0.3 g, 479 μmol). m/z (ESI-pos) M+1=626.4/628.4

Step G: To a solution of tert-butyl (S)-3-amino-2-[m-({2-[2-bromo-5-(2-oxo-1,3-oxazolidin-3-yl)phenyl]ethoxy}methyl)phenyl]-4-methyl-1,2,5-triaza-2,4,6,7-tetrahydro-5H-indene-5-carboxylate (300 mg, 479 μmol) and N-ethyldiisopropylamine (250 μL, 1.44 mmol) in tetrahydrofuran (4.79 mL) was added benzenyl chloroformate (120 μL, 958 μmol), and the reaction was stirred at 25° C. for 10 minutes. The reaction was concentrated in vacuo to afford tert-butyl (S)-2-[m-({2-[2-bromo-5-(2-oxo-1,3-oxazolidin-3-yl)phenyl]ethoxy}methyl)phenyl]-4-methyl-3-[phenyl(oxycarbonylamino)]-1,2,5-triaza-2,4,6,7-tetrahydro-5H-indene-5-carboxylate (358 mg, 479 μmol) which was used directly in the next step. m/z (ESI-pos) M+1=746.4/748.4

Step H: Crude tert-butyl (S)-2-[m-({2-[2-bromo-5-(2-oxo-1,3-oxazolidin-3-yl)phenyl]ethoxy}methyl)phenyl]-4-methyl-3-[phenyl(oxycarbonylamino)]-1,2,5-triaza-2,4,6,7-tetrahydro-5H-indene-5-carboxylate (358 mg, 479 μmol) was dissolved in pyridine (2.4 mL), to which 2,2-dimethoxyethylamine (157 μL, 1.44 mmol) was added. The mixture was stirred at room temperature for 1 hour. The reaction mixture was concentrated in vacuo then purified via column chromatography eluting with 5-100% EtOAc/Heptane to afford tert-butyl (S)-2-[m-({2-[2-bromo-5-(2-oxo-1,3-oxazolidin-3-yl)phenyl]ethoxy}methyl)phenyl]-3-[3-(2,2-dimethoxyethyl)ureido]-4-methyl-1,2,5-triaza-2,4,6,7-tetrahydro-5H-indene-5-carboxylate (287 mg, 379 μmol). m/z (ESI-pos) M+1=757.5/759.5

Step I: To a solution of tert-butyl (S)-2-[m-({2-[2-bromo-5-(2-oxo-1,3-oxazolidin-3-yl)phenyl]ethoxy}methyl)phenyl]-3-[3-(2,2-dimethoxyethyl)ureido]-4-methyl-1,2,5-triaza-2,4,6,7-tetrahydro-5H-indene-5-carboxylate (387 mg, 511 μmol) in tetrahydrofuran (5.11 mL) was added methanesulfonic acid (166 μL, 2.55 mmol). The mixture was stirred at 60° C. for 30 minutes. Aqueous saturated sodium bicarbonate was added until bubbling ceased (~pH=8), and then tert-butyl-tert-butyl(oxycarbonyloxy)formylate (111 mg, 511 μmol) was added, and the resulting mixture was stirred at room temperature for 1 hour. The reaction mixture was diluted with ethyl acetate and washed with water twice. The organic layer was then dried over anhydrous Na₂SO₄, filtered and the filtrate was evaporated under reduced pressure. The crude residue was purified via column chromatography eluting with 0-6% MeOH/DCM to afford tert-butyl (S)-2-[m-({2-[2-bromo-5-(2-oxo-1,3-oxazolidin-3-yl)phenyl]ethoxy}methyl)phenyl]-4-methyl-3-(2-oxo-2,3-dihydro-1H-imidazol-1-yl)-1,2,5-triaza-2,4,6,7-tetrahydro-5H-indene-5-carboxylate (248 mg, 358 μmol). m/z (ESI-pos) M+1=693.3/695.3

Step J: A solution of tert-butyl (S)-2-[m-({2-[2-bromo-5-(2-oxo-1,3-oxazolidin-3-yl)phenyl]ethoxy}methyl)phenyl]-4-methyl-3-(2-oxo-2,3-dihydro-1H-imidazol-1-yl)-1,2,5-triaza-2,4,6,7-tetrahydro-5H-indene-5-carboxylate (248 mg, 358 μmol), dipotassium carbonate (148 mg, 1.07 mmol), copper iodide (68.1 mg, 358 μmol), and (1R,2R)-1,2-bis(methylamino)cyclohexane (84.6 μL, 536 μmol) in NMP (10.9 mL) was sparged with nitrogen for 5 minutes. The solution was stirred at 130° C. for 3 hours. The solution was allowed to cool to ambient temperature and diluted with water. The mixture was then extracted with EtOAc twice. The combined organic layers were washed with brine, dried over Na₂SO₄, filtered and concentrated. The crude product was then purified via column chromatography on silica eluting with 10-100% EtOAc/Heptane to afford tert-butyl (S)-8-methyl-30-oxo-18-(2-oxo-1,3-oxazolidin-3-yl)-23-oxa-2,3,7,11,14-pentaazahexacyclo[23.3.1.1¹¹,¹⁴.0²,¹⁰.0⁴.⁹.0¹⁵,²⁰]triaconta-1(28),3,9,12,15(20),16,18,25(29),26-nonaene-7-carboxylate (79 mg, 129 μmol). m/z (ESI-pos) M+1=613.4

Step K: Trifluoroacetic acid (0.7 mL, 9.15 mmol) was added to a stirring solution of tert-butyl (S)-8-methyl-30-oxo-18-(2-oxo-1,3-oxazolidin-3-yl)-23-oxa-2,3,7,11,14-pentaazahexacyclo[23.3.1.1¹¹,¹⁴.0²,¹⁰.0⁴.⁹.0¹⁵,²⁰]triaconta-1(28),3,9,12,15(20),16,18,25(29),26-nonaene-7-carboxylate (79 mg, 129 μmol) in dichloromethane (5 mL). After an hour, the reaction was partitioned between DCM and 2M aqueous Na₂CO₃. Organics collected, washed again with 2M aqueous Na₂CO₃, dried over magnesium sulfate, filtered and concentrated in vacuo to afford (S)-8-methyl-18-(2-oxo-1,3-oxazolidin-3-yl)-23-oxa-2,3,7,11,14-pentaazahexacyclo[23.3.1.1¹¹,¹⁴.0²,¹⁰.0⁴.⁹.0¹⁵,²⁰]triaconta-1(28),3,9,12,15(20),16,18,25(29),26-nonaen-30-one (47.4 mg, 92.5 μmol). m/z (ESI-pos) M+1=513.4

Step L: An 8 mL vial was charged with(S)-8-methyl-18-(2-oxo-1,3-oxazolidin-3-yl)-23-oxa-2,3,7,11,14-pentaazahexacyclo[23.3.1.1¹¹,¹⁴.0²,¹⁰.0⁴.⁹.0¹⁵,²⁰]triaconta-1(28),3,9,12,15(20),16,18,25(29),26-nonaen-30-one (24 mg, 46.8 μmol), dimethylformamide (468 μL), and N-ethyldiisopropylamine (24.5 μL, 140 μmol). 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (38.5 mg, 93.6 μmol) was then added followed by [(dimethylamino)(3-hydroxy-1λ⁵,2,3,4-tetraaza-3H-inden-1-yl)methylene]bis(methyl)azanium hexafluoridophosphate (1-) (37.5 mg, 98.3 μmol) and the mixture was stirred at room temperature for 20 hours. Reaction was transferred with MeCN/H₂O and purified directly by reverse phase prep-HPLC purification eluting with 40-100% MeCN+0.1% formic acid in H₂O+0.1% formic acid. Desired fractions were lyophilized overnight to afford (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-18-(2-oxo-1,3-oxazolidin-3-yl)-23-oxa-2,3,7,11,14-pentaazahexacyclo[23.3.1.1¹¹,¹⁴.0²,¹⁰.0⁴.⁹.0¹⁵,²⁰]triaconta-1(28),3,9,12,15(20),16,18,25(29),26-nonaen-30-one (18.9 mg, 20.9 μmol). m/z (ESI-pos) M+1=906.6; ¹H NMR (400 MHz, CHLOROFORM-D) δ 11.28 (m, 1H), 7.70-7.43 (m, 6H), 7.37-6.97 (m, 4H), 6.78-6.66 (m, 1H), 6.48-5.92 (m, 2H), 5.92-5.21 (m, 1H), 4.95-4.39 (m, 5H), 4.13-2.71 (m, 11H), 1.98-0.99 (m, 20H).

### Example 185—Synthesis of Compound 185

### Procedure for Synthesis of Compound 185

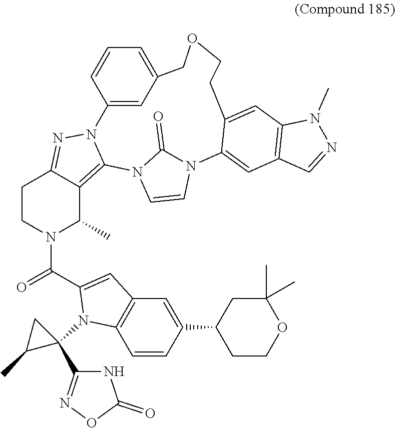 [SMILES: C[C@H]1c2c(nn3c2-n2ccn(c2=O)-c2cc4cnn(C)c4cc2CCOCc2cccc-3c2)CCN1C(=O)c1cc2cc([C@H]3CCOC(C)(C)C3)ccc2n1[C@@]1(c2noc(=O)[nH]2)C[C@@H]1C]

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-20-methyl-26-oxa-2,3,7, 11, 14, 19,20-heptaazaheptacyclo[26.3.1.1¹¹,¹⁴.0²,¹⁰.0⁴,⁹.0¹⁵,²³.0¹⁷,²¹]tritriaconta-1(32),3,9,12,15,17(21), 18,22,28,30-decaen-33-one

Compound 185 was synthesized following the procedure of Example 184, with 3-[4-bromo-3-(2-hydroxyethyl)phenyl]-1,3-oxazolidin-2-one being replaced with 2-(5-bromo-1-methyl-1H-indazol-6-yl) ethanol. m/z (ESI-pos) M+1=875.6; ¹H NMR (400 MHz, CHLOROFORM-D) δ 11.34-11.24 (m, 1H), 8.01-7.07 (m, 9H), 7.01-5.94 (m, 3H), 5.94-5.23 (m, 1H), 4.95-4.42 (m, 3H), 4.11-2.82 (m, 14H), 2.03-0.85 (m, 19H).

### Example 186—Synthesis of Compound 186

### Synthetic Scheme

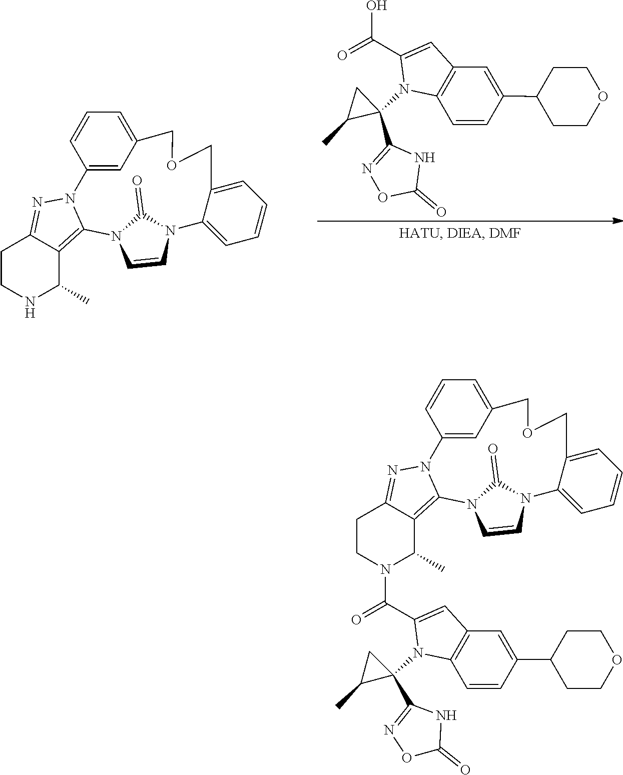

### Procedure for Synthesis of Compound 186

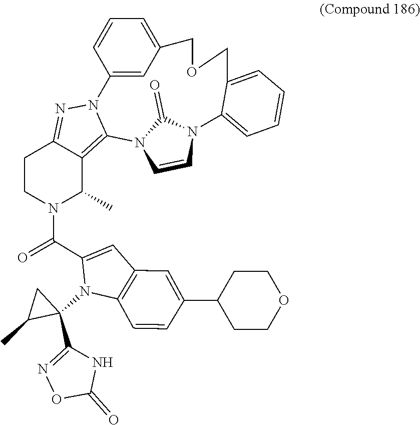

### (8S,10M)-8-methyl-7-({1-[(1S,2S)-2-methyl-1-(5-oxo-4,5-dihydro-1,2,4-oxadiazol-3-yl)cyclopropyl]-5-(tetrahydro-4H-pyran-4-yl)-2-indolyl}carbonyl)-22-oxa-2,3,7,11,14-pentaazahexacyclo[22.3.1.1¹¹,¹⁴.0²,¹⁰.0⁴.⁹.0¹⁵,²⁰]nonacosa-1(28),3,9,12,15,17,19,24,26-nonaen-29-one

Step A: An 8 mL vial was charged with(S)-8-methyl-22-oxa-2,3,7,11,14-pentaazahexacyclo[22.3.1.1¹¹,¹⁴.0²,¹⁰.0⁴.⁹.0¹⁵,²⁰]nonacosa-1(27),3,9,12,15(20),16,18,24(28),25-nonaen-29-one-hydrogen chloride (1/1) (20 mg, 44.5 μmol), 1-[(1S,2S)-2-methyl-1-(5-oxo-4,5-dihydro-1,2,4-oxadiazol-3-yl)cyclopropyl]-5-(tetrahydro-4H-pyran-4-yl)-2-indolecarboxylic acid (22.2 mg, 57.8 μmol), dimethylformamide (445 μL), and N-ethyldiisopropylamine (30.4 μL, 178 μmol) followed by addition of HATU (27.1 mg, 71.1 μmol) and the mixture was stirred at room temperature for 16 hours. The reaction was transferred with MeCN and purified directly by reverse phase prep-HPLC purification eluting with 40-100% MeCN+0.1% formic acid in water+0.1% formic acid. Desired fractions were concentrated in vacuo to afford (8S,10M)-8-methyl-7-({1-[(1S,2S)-2-methyl-1-(5-oxo-4,5-dihydro-1,2,4-oxadiazol-3-yl)cyclopropyl]-5-(tetrahydro-4H-pyran-4-yl)-2-indolyl}carbonyl)-22-oxa-2,3,7,11,14-pentaazahexacyclo[22.3.1.1¹¹,¹⁴.0²,¹⁰.0⁴.⁹.0¹⁵,²⁰]nonacosa-1(28),3,9,12,15,17,19,24,26-nonaen-29-one (25.4 mg, 32.6 μmol). m/z (ESI-pos) M+1=779.6; ¹H NMR (400 MHz, CHLOROFORM-D) δ 11.37-11.26 (m, 1H), 7.69-7.15 (m, 11H), 6.82-6.70 (m, 1H), 6.47-5.85 (m, 2H), 5.86-5.24 (m, 1H), 4.95-4.06 (m, 7H), 3.70-2.81 (m, 6H), 1.95-1.11 (m, 13H).

### Example 187—Synthesis of Compound 187

### Synthetic Scheme

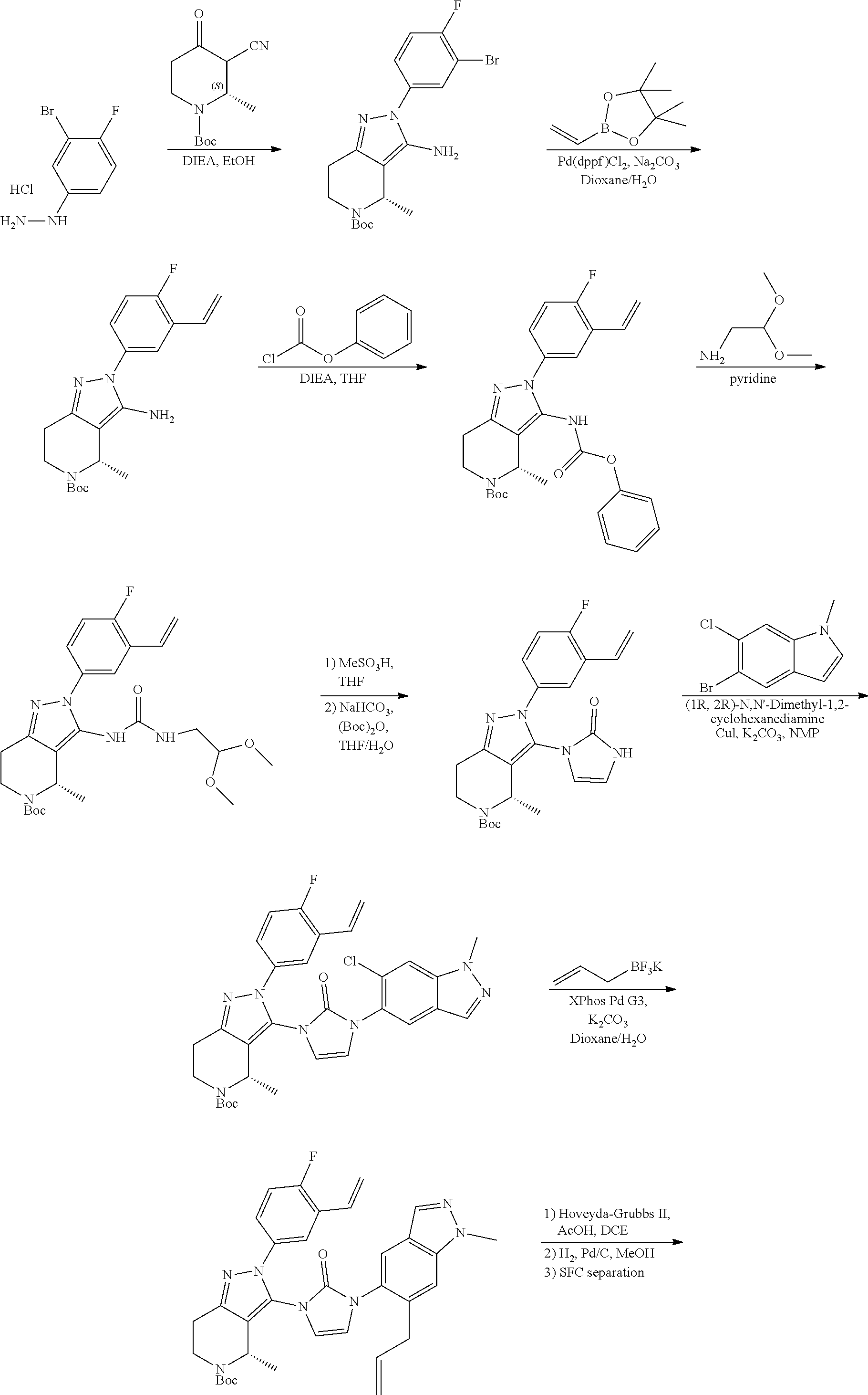

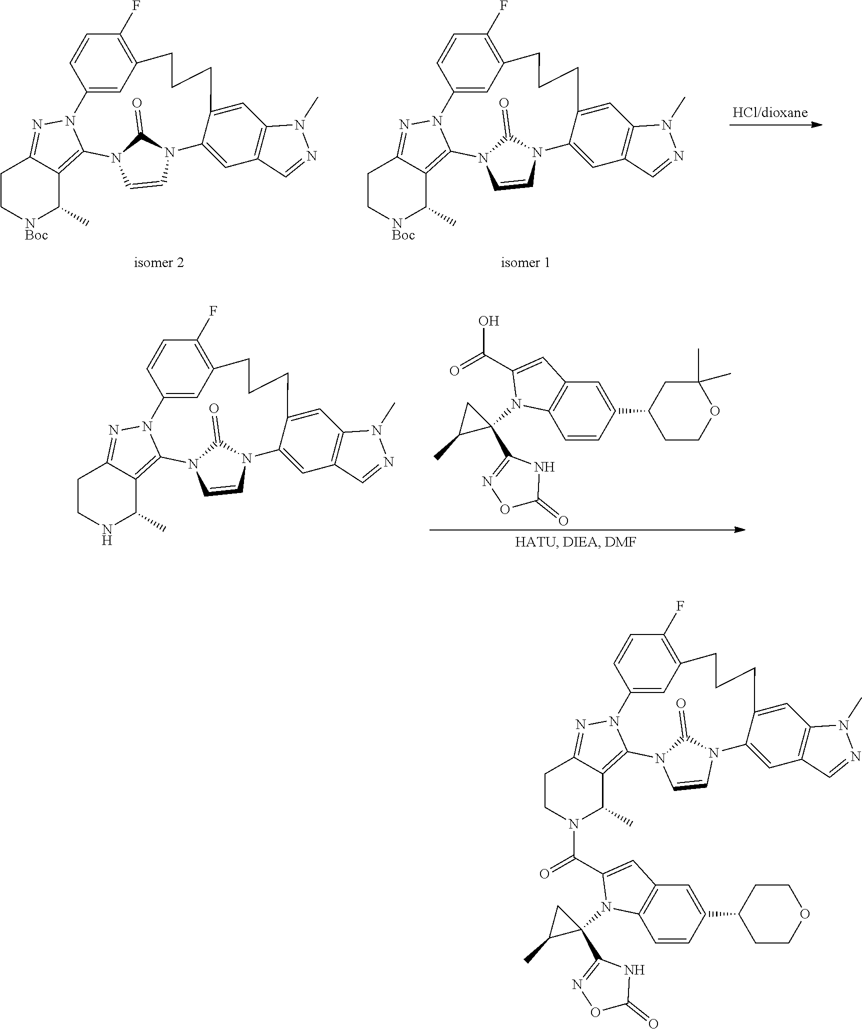

### Procedure for Synthesis of Compound 187

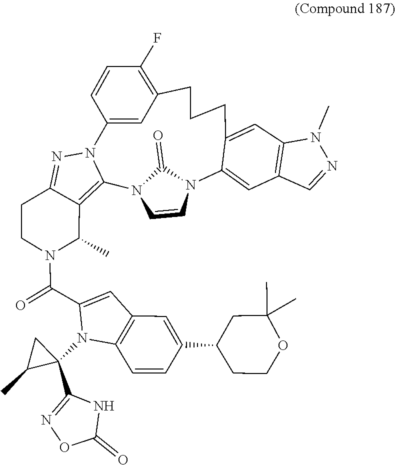

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-28-fluoro-8-methyl-20-methyl-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21), 18,22,27,29-decaen-32-one

Step A: To a 500 mL flask was added tert-butyl(2S)-3-cyano-2-methyl-4-oxo-1-piperidinecarboxylate (5 g, 21 mmol), 3-bromo-4-fluorophenylaminoammonium chloride (5.07 g, 21 mmol), N-ethyldiisopropylamine (4.75 mL, 27.3 mmol), and ethanol (83.9 mL). The reaction was fitted with a reflux condenser and heated in a heating block to 90° C. for 20 hours. The reaction was cooled and concentrated in vacuo. The residue was partitioned between EtOAc and water. Organics were collected and the aqueous layer was extracted twice more with EtOAc. Combined organics were washed again with water, then brine, dried over sodium sulfate, filtered and concentrated in vacuo to afford tert-butyl (S)-3-amino-2-(3-bromo-4-fluorophenyl)-4-methyl-1,2,5-triaza-2,4,6,7-tetrahydro-5H-indene-5-carboxylate (9.24 g, 21.7 mmol) which was used in the next step without purification. m/z (ESI-pos) M+1=425.2/427.2

Step B: To a mixture of tert-butyl (S)-3-amino-2-(3-bromo-4-fluorophenyl)-4-methyl-1,2,5-triaza-2,4,6,7-tetrahydro-5H-indene-5-carboxylate (8.92 g, 21 mmol), disodium carbonate (4.45 g, 41.9 mmol), water (41.9 mL), 1,4-dioxane (105 mL) was added Pd (dppf) C₁₂ (856 mg, 1.05 mmol) and 4,4,5,5-tetramethyl-2-vinyl-1,3,2-dioxaborolane (10.7 mL, 62.9 mmol). The solution was purged with nitrogen for 10 minutes, fitted with a reflux condenser, and the resulting mixture was stirred at 85° C. for 24 hours. The reaction mixture was cooled down to room temperature and diluted with water. The aqueous mixture was extracted with ethyl acetate three times. The combined organic layers were washed with brine solution, dried over anhydrous Na₂SO₄, filtered and the filtrate was evaporated under reduced pressure to give the crude compound. The crude compound was purified by silica column chromatography eluting with 10-60% EtOAc/heptane to afford tert-butyl (S)-3-amino-2-(4-fluoro-3-styrenyl)-4-methyl-1,2,5-triaza-2,4,6,7-tetrahydro-5H-indene-5-carboxylate (6.08 g, 16.3 mmol). m/z (ESI-pos) M+1=373.2

Step C: To a solution of tert-butyl (S)-3-amino-2-(4-fluoro-3-styrenyl)-4-methyl-1,2,5-triaza-2,4,6,7-tetrahydro-5H-indene-5-carboxylate (6.08 g, 16.3 mmol) and N-ethyldiisopropylamine (8.53 mL, 49 mmol) in tetrahydrofuran (163 mL) was added benzenyl chloroformate (4.1 mL, 32.6 mmol) and the reaction was stirred at room temperature for 30 minutes. The reaction was concentrated in vacuo to afford tert-butyl (S)-2-(4-fluoro-3-styrenyl)-4-methyl-3-[phenyl(oxycarbonylamino)]-1,2,5-triaza-2,4,6,7-tetrahydro-5H-indene-5-carboxylate (8.04 g, 16.3 mmol) which was used directly in the next step. m/z (ESI-pos) M+1=493.4

Step D: Crude tert-butyl (S)-2-(4-fluoro-3-styrenyl)-4-methyl-3-[phenyl(oxycarbonylamino)]-1,2,5-triaza-2,4,6,7-tetrahydro-5H-indene-5-carboxylate (8.04 g, 16.3 mmol) was dissolved in pyridine (54.4 mL, 676 mmol) to which 2,2-dimethoxyethylamine (5.34 mL, 49 mmol) was added. The mixture was stirred at room temperature for 10 minutes. The reaction mixture was concentrated in vacuo, dissolved in DCM, filtered, and directly loaded onto a silica cartridge eluting with 20-100% EtOAc/Heptane to afford tert-butyl (S)-3-[3-(2,2-dimethoxyethyl)ureido]-2-(4-fluoro-3-styrenyl)-4-methyl-1,2,5-triaza-2,4,6,7-tetrahydro-5H-indene-5-carboxylate (4.35 g, 8.64 mmol). m/z (ESI-pos) M+1=504.4

Step E: To a solution of tert-butyl (S)-3-[3-(2,2-dimethoxyethyl)ureido]-2-(4-fluoro-3-styrenyl)-4-methyl-1,2,5-triaza-2,4,6,7-tetrahydro-5H-indene-5-carboxylate (4.35 g, 8.64 mmol) in tetrahydrofuran (43.2 mL) was added methanesulfonic acid (2.8 mL, 43.2 mmol). The mixture was stirred at 60° C. for 30 minutes. Aqueous saturated sodium bicarbonate was added until bubbling ceased (pH=8), and then tert-butyl-tert-butyl(oxycarbonyloxy) formylate (2.83 g, 13 mmol) was added, and the resulting mixture was stirred at room temperature for 45 minutes. The reaction mixture was diluted with ethyl acetate and washed with water twice. The organic layer was then dried over anhydrous Na₂SO₄, filtered, and the filtrate was evaporated under reduced pressure. The crude residue was purified via column chromatography eluting with 0-7% MeOH/DCM to afford tert-butyl (S)-2-(4-fluoro-3-styrenyl)-4-methyl-3-(2-oxo-2,3-dihydro-1H-imidazol-1-yl)-1,2,5-triaza-2,4,6,7-tetrahydro-5H-indene-5-carboxylate (3.36 g, 7.65 mmol). m/z (ESI-pos) M+1=440.3

Step F: To a solution of tert-butyl (S)-2-(4-fluoro-3-styrenyl)-4-methyl-3-(2-oxo-2,3-dihydro-1H-imidazol-1-yl)-1,2,5-triaza-2,4,6,7-tetrahydro-5H-indene-5-carboxylate (750 mg, 1.71 mmol) and 5-bromo-6-chloro-1-methyl-1H-indazole (754 mg, 3.07 mmol) in N-methyl-2-pyrrolidone (8.53 mL) were added (1S,2S)-1,2-bis(methylamino)cyclohexane (273 μL, 1.71 mmol), copper iodide (163 mg, 853 μmol) and dipotassium carbonate (708 mg, 5.12 mmol). The reaction mixture was sparged with nitrogen for 5 minutes, then sealed with parafilm and heated at 100° C. for 16 hours. The reaction mixture was cooled to ambient temperature and diluted with ethyl acetate and washed with aqueous saturated ammonium chloride solution twice. The organic layer was dried over anhydrous Na₂SO4 and evaporated under reduced pressure. The crude residue was purified via column chromatography eluting with 10-100% EtOAc/Heptane to afford tert-butyl (S)-3-[3-(6-chloro-1-methyl-1H-indazol-5-yl)-2-oxo-2,3-dihydro-1H-imidazol-1-yl]-2-(4-fluoro-3-styrenyl)-4-methyl-1,2,5-triaza-2,4,6,7-tetrahydro-5H-indene-5-carboxylate (536 mg, 887 μmol). m/z (ESI-pos) M+1=604.4

Step G: 1,4-Dioxane (3.97 mL) was added to a vial containing tert-butyl (S)-3-[3-(6-chloro-1-methyl-1H-indazol-5-yl)-2-oxo-2,3-dihydro-1H-imidazol-1-yl]-2-(4-fluoro-3-styrenyl)-4-methyl-1,2,5-triaza-2,4,6,7-tetrahydro-5H-indene-5-carboxylate (480 mg, 795 μmol), XPhos Pd G3 (67.3 mg, 79.5 μmol), potassium allyltris(fluoro)boranuide (294 mg, 1.99 mmol), dipotassium carbonate (329 mg, 2.38 mmol), and water (1 mL). The mixture was sparged with nitrogen for 5 minutes, then heated to 80° C. for 1.5 hours. Reaction was concentrated in vacuo and loaded onto a silica cartridge eluting with 20-100% EtOAc/Heptane. Material repurified by ACCQ prep-hplc eluting with 35-85% MeCN+0.1% formic acid in water+0.1% formic acid. Desired fractions concentrated in vacuo to afford tert-butyl (S)-3-[3-(6-allyl-1-methyl-1H-indazol-5-yl)-2-oxo-2,3-dihydro-1H-imidazol-1-yl]-2-(4-fluoro-3-styrenyl)-4-methyl-1,2,5-triaza-2,4,6,7-tetrahydro-5H-indene-5-carboxylate (160 mg, 262 μmol). m/z (ESI-pos) M+1=610.5

Step H: To a 250 mL flask was added tert-butyl (S)-3-[3-(6-allyl-1-methyl-1H-indazol-5-yl)-2-oxo-2,3-dihydro-1H-imidazol-1-yl]-2-(4-fluoro-3-styrenyl)-4-methyl-1,2,5-triaza-2,4,6,7-tetrahydro-5H-indene-5-carboxylate (160 mg, 262 μmol), 1,2-dichloroethane (160 mL), and acetic acid (15 μL, 262 μmol). The mixture was sparged with nitrogen for 10 minutes, then ruthenium o-isopropoxymethanediidyltoluene 1,3-bis(mesityl)-2,2-imidazolidinediide dichloride (41.1 mg, 65.6 μmol) was added and the reaction was heated to 55° C. while sparging with nitrogen for 20 hours. The reaction was concentrated in vacuo and purified via column chromatography eluting with 10-100% EtOAc/Heptane to afford tert-butyl (S)-28-fluoro-8-methyl-20-methyl-32-oxo-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(30),3,9,12,15 (23),16,18,21,25,27 (31),28-undecaene-7-carboxylate (153 mg, 263 μmol) as a mixture of alkene isomers. m/z (ESI-pos) M+1=582.4

Step I: Tert-butyl (S)-28-fluoro-8-methyl-20-methyl-32-oxo-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(30),3,9,12,15 (23),16,18,21,25,27 (31),28-undecaene-7-carboxylate (160 mg, 275 μmol) was dissolved in MeOH (5 mL), 10% palladium on carbon (50 mg, 470 μmol) was added, and the mixture was sparged with hydrogen for one minute, then left stirring under hydrogen balloon atmosphere for 5 hours. The reaction was diluted with DCM, filtered, and concentrated in vacuo. Crude residue was purified by ACCQ prep-HPLC purification eluting with 40-100% MeCN+0.1% formic acid in water+0.1% formic acid, desired fractions concentrated in vacuo. The mixture of atropisomers was purified by chiral SFC, Daicel ChiralPak IC (150×21 mm), 35° C., 35% ethanol, 65 mL/min, to afford tert-butyl (8S,10M)-28-fluoro-8-methyl-20-methyl-32-oxo-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21),18,22,27,29-decaene-7-carboxylate (isomer 1, 15 mg, 25.7 μmol) and tert-butyl (8S,10P)-28-fluoro-8-methyl-20-methyl-32-oxo-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21),18,22,27,29-decaene-7-carboxylate (isomer 2, 16 mg, 27.4 μmol). m/z (ESI-pos) M+1=584.4

Step J: 4M HCl in Dioxane (0.5 mL) was added to a stirring solution of tert-butyl (8S,10M)-28-fluoro-8-methyl-20-methyl-32-oxo-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21),18,22,27,29-decaene-7-carboxylate (isomer 1, 7.5 mg, 12.8 μmol) in dichloromethane (0.5 mL). The reaction was stirred at room temperature for 45 minutes, then concentrated in vacuo to afford (8S,10M)-28-fluoro-8-methyl-20-methyl-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21),18,22,27,29-decaen-32-one hydrochloride (6.68 mg, 12.8 μmol).

Step K: An 8 mL vial was charged with (8S,10M)-28-fluoro-8-methyl-20-methyl-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21),18,22,27,29-decaen-32-one hydrochloride (6.68 mg, 12.8 μmol), 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (6.87 mg, 16.7 μmol), dimethylformamide (0.5 mL), and N-ethyldiisopropylamine (8.95 μL, 51.4 μmol) followed by addition of HATU (7.82 mg, 20.6 μmol) and the mixture was stirred at room temperature for 16 hours. The reaction was transferred with MeCN and purified directly by reverse phase prep-HPLC purification eluting with 40-100% MeCN+0.1% formic acid in water+0.1% formic acid. Desired fractions were concentrated in vacuo to afford (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-28-fluoro-8-methyl-20-methyl-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21),18,22,27,29-decaen-32-one (8.17 mg, 9.32 μmol). m/z (ESI-pos) M+1=877.6; ¹H NMR (400 MHz, CHLOROFORM-D) δ 11.30 (m, 1H), 8.07-7.01 (m, 9H), 6.82-6.54 (m, 1H), 6.32-5.84 (m, 2H), 5.85-5.22 (m, 1H), 4.96-3.11 (m, 8H), 3.10-0.98 (m, 27H).

### Example 188—Synthesis of Compound 188

### Procedure for Synthesis of Compound 188

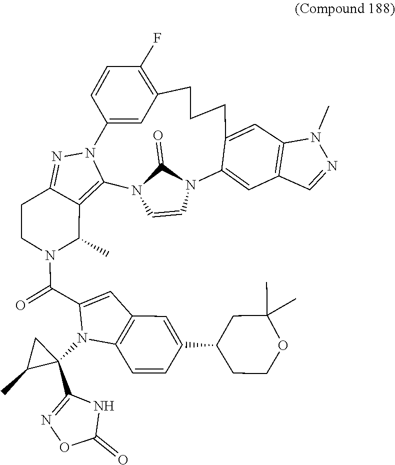

### (8S,10P)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-28-fluoro-8-methyl-20-methyl-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21), 18,22,27,29-decaen-32-one

Compound 188 was synthesized following the procedure of Example 187, with tert-butyl (8S,10M)-28-fluoro-8-methyl-20-methyl-32-oxo-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21),18,22,27,29-decaene-7-carboxylate (isomer 1) being replaced with tert-butyl (8S,10P)-28-fluoro-8-methyl-20-methyl-32-oxo-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21),18,22,27,29-decaene-7-carboxylate (isomer 2). m/z (ESI-pos) M+1=877.6; ¹H NMR (400 MHz, CHLOROFORM-D) δ 11.30-11.23 (m, 1H), 8.08-7.11 (m, 9H), 6.76-6.68 (m, 1H), 6.31-6.05 (m, 2H), 5.93-5.22 (m, 1H), 4.94-4.45 (m, 1H), 4.15-4.02 (m, 3H), 3.97-2.69 (m, 7H), 2.55-0.80 (m, 24H).

### Example 189—Synthesis of Compound 189

### Synthetic Scheme

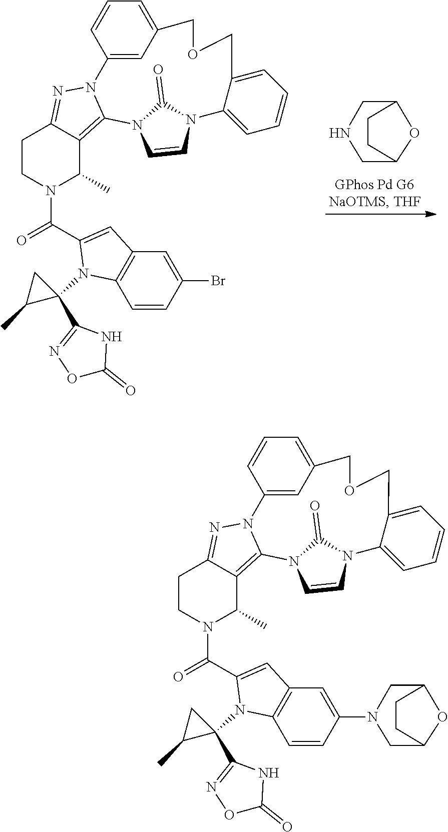

### Procedure for Synthesis of Compound 189

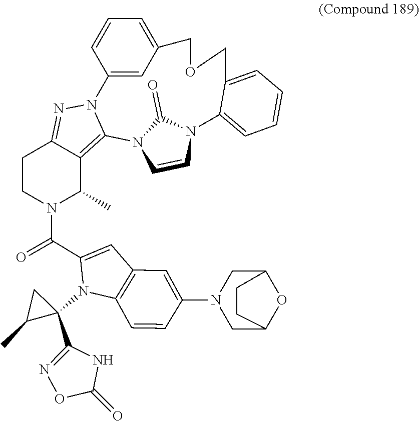

### (8S,10M)-8-methyl-7-({1-[(1S,2S)-2-methyl-1-(5-oxo-4,5-dihydro-1,2,4-oxadiazol-3-yl)cyclopropyl]-5-(8-oxa-3-azabicyclo[3.2.1]oct-3-yl)-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: A 4 mL vial was charged with (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (15 mg, 19.4 μmol), 8-oxa-3-azabicyclo[3.2.1]octane (4.39 mg, 38.8 μmol), GPhos Pd G6 (1.83 mg, 1.94 μmol), sodium trimethylsilanolate (6.96 mg, 62 μmol), followed by addition of tetrahydrofuran (485 μL). The mixture was sparged with nitrogen for 5 minutes then heated to 70° C. After 16 hours, the reaction was cooled and partitioned between saturated aqueous ammonium chloride and ethyl acetate. The aqueous was extracted twice with ethyl acetate, then the organics were dried over sodium sulfate, filtered, and concentrated in vacuo. The crude residue was purified by ACCQ reverse phase prep-HPLC eluting with 30-80% MeCN+0.1% formic acid in water+0.1% formic acid. The desired fractions were lyophilized overnight to afford (8S,10M)-8-methyl-7-({1-[(1S,2S)-2-methyl-1-(5-oxo-4,5-dihydro-1,2,4-oxadiazol-3-yl)cyclopropyl]-5-(8-oxa-3-azabicyclo[3.2.1]oct-3-yl)-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (4.44 mg, 5.1 μmol). m/z (ESI-pos) M+1=806.6; ¹H NMR (400 MHz, CHLOROFORM-D) δ 11.35-11.26 (m, 1H), 7.69-7.30 (m, 7H), 7.26-7.14 (m, 2H), 7.08-6.96 (m, 2H), 6.76-6.60 (m, 1H), 6.47-5.82 (m, 2H), 5.83-5.21 (m, 1H), 4.92-4.65 (m, 2H), 4.60-4.42 (m, 3H), 4.34-4.21 (m, 1H), 4.14-4.05 (m, 1H), 3.70-3.36 (m, 1H), 3.34-2.98 (m, 6H), 2.04-1.08 (m, 13H).

### Example 190—Synthesis of Compound 190

### Procedure for Synthesis of Compound 190

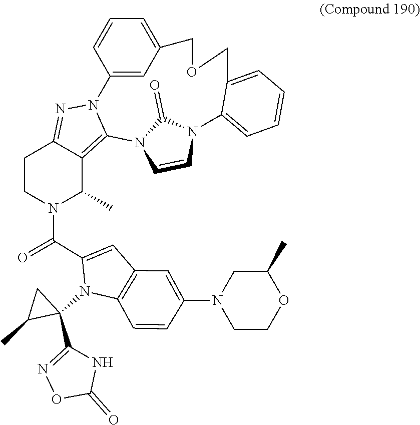

### (8S,10M)-8-methyl-7-({5-[(R)-2-methyl-4-morpholinyl]-1-[(1S,2S)-2-methyl-1-(5-oxo-4,5-dihydro-1,2,4-oxadiazol-3-yl)cyclopropyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 190 was synthesized following the procedure of Example 189, with 8-oxa-3-azabicyclo[3.2.1]octane being replaced with (R)-2-methylmorpholine. m/z (ESI-pos) M+1=794.6; ¹H NMR (400 MHz, CHLOROFORM-D) δ 11.36-11.32 (m, 1H), 7.68-7.33 (m, 7H), 7.28-7.09 (m, 4H), 6.75-6.63 (m, 1H), 6.48-5.85 (m, 2H), 5.86-5.23 (m, 1H), 4.94-4.44 (m, 3H), 4.35-4.22 (m, 1H), 4.16-3.58 (m, 5H), 3.45-3.31 (m, 2H), 3.22-2.99 (m, 2H), 2.90-2.79 (m, 1H), 2.55-2.45 (m, 1H), 1.93-1.10 (m, 11H).

### Example 191—Synthesis of Compound 191

### Procedure for Synthesis of Compound 191

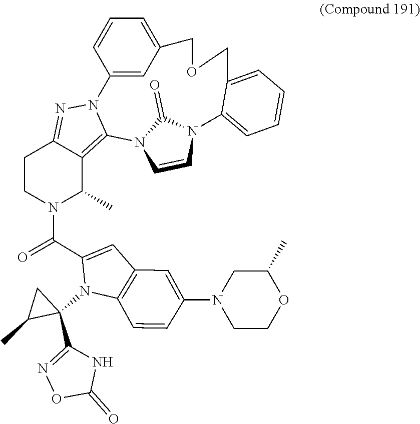

### (8S,10M)-7-({5-[(S)-2-methyl-4-morpholinyl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 191 was synthesized following the procedure of Example 189, with 8-oxa-3-azabicyclo[3.2.1]octane being replaced with(S)-2-methylmorpholine. m/z (ESI-pos) M+1=794.6; ¹H NMR (400 MHz, CHLOROFORM-D) δ 11.46-11.22 (m, 1H), 7.69-7.32 (m, 7H), 7.25-7.10 (m, 4H), 6.77-6.64 (m, 1H), 6.49-5.85 (m, 2H), 5.86-5.24 (m, 1H), 4.95-4.44 (m, 3H), 4.36-4.22 (m, 1H), 4.15-3.58 (m, 5H), 3.46-3.29 (m, 2H), 3.23-2.99 (m, 2H), 2.89-2.78 (m, 1H), 2.56-2.46 (m, 1H), 1.93-1.10 (m, 12H).

### Example 192—Synthesis of Compound 192

### Synthetic Scheme

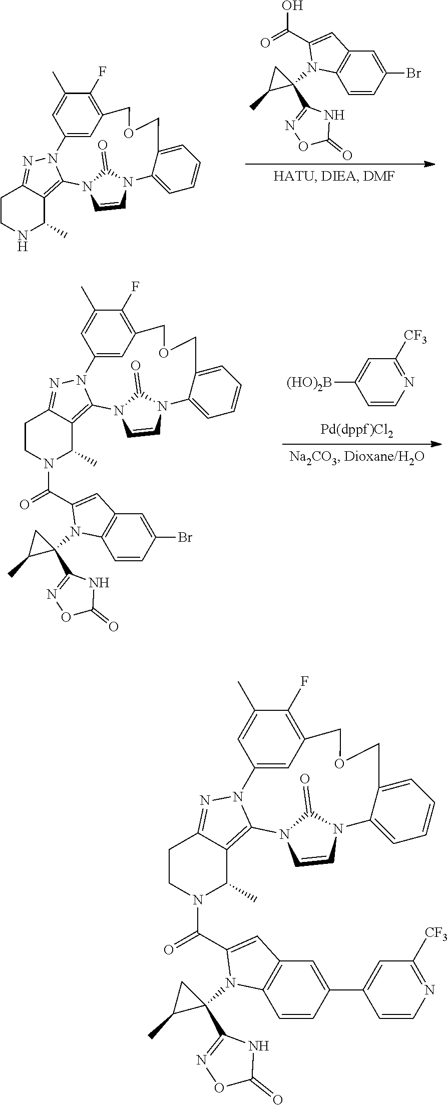

### Procedure for Synthesis of Compound 192

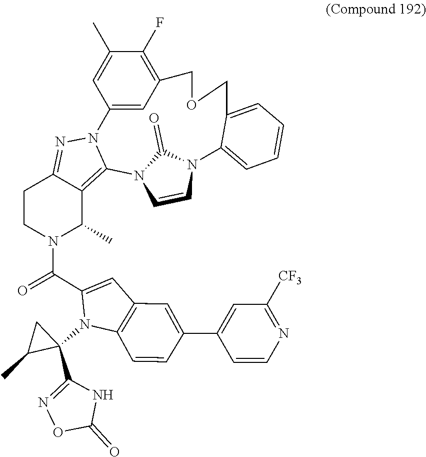

### (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (8S,10M)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (0.5 g, 1.04 mmol) and 5-bromo-1-[(1S,2S)-2-methyl-1-(5-oxo-4,5-dihydro-1,2,4-oxadiazol-3-yl)cyclopropyl]-2-indolecarboxylic acid (432 mg, 1.14 mmol), in dimethylformamide (3.46 mL) was added N-ethyldiisopropylamine (723 μL, 4.15 mmol), and HATU (552 mg, 1.45 mmol). The mixture stirred at room temperature for 16 hours. The reaction mixture was directly purified by reverse phase column prep-HPLC eluting with 40 to 100% MeCN/H₂O (with 0.1% formic acid buffer), pure fractions were collected and lyophilized to afford (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (655 mg, 813 μmol). m/z (ESI-pos) M+1=805.4/807.4.

Step B: A solution of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (15 mg, 18.6 μmol) Pd(dppf)Cl₂ (1.52 mg, 1.86 μmol), [2-(trifluoromethyl)-4-pyridyl]boranediol (10.7 mg, 55.9 μmol), and disodium carbonate (9.87 mg, 93.1 μmol) in 1,4-dioxane (465 μL) and water (186 μL) was heated to 80° C. for 1 hour. The solution was partitioned between EtOAc and water, the organic layer was dried with Na₂SO₄, filtered and concentrated. The crude residue was purified by reverse-phase prep-HPLC (40-100% MeCN/water with 0.1% formic acid) and the product was lyophilized to afford (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (9.44 mg, 10.8 μmol). m/z (ESI-pos) M+1=872.5; ¹H NMR (400 MHz, CHLOROFORM-D) δ 11.35-11.19 (m, 1H), 8.81-8.75 (m, 1H), 8.06-7.92 (m, 2H), 7.83-7.64 (m, 3H), 7.60-7.31 (m, 5H), 7.16-7.01 (m, 1H), 6.95-6.82 (m, 1H), 6.48-5.88 (m, 2H), 5.87-5.22 (m, 1H), 4.99-4.42 (m, 3H), 4.34-4.12 (m, 2H), 3.74-3.40 (m, 1H), 3.24-3.00 (m, 2H), 2.37-2.30 (m, 3H), 2.02-1.14 (m, 9H).

### Example 193—Synthesis of Compound 193

### Procedure for Synthesis of Compound 193

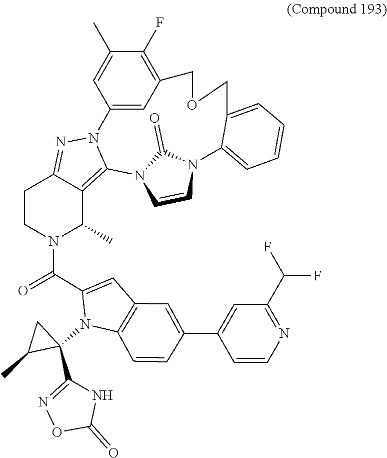

### (8S,10M)-7-({5-[2-(difluoromethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 193 was synthesized following the procedure of Example 192, with [2-(trifluoromethyl)-4-pyridyl]boranediol being replaced with 2-[2-(difluoromethyl)-4-pyridyl]-4,4,5,5-tetramethyl-1,3,2-dioxaborolane. m/z (ESI-pos) M+1=854.6; ¹H NMR (400 MHz, CHLOROFORM-D) δ 11.35-11.20 (m, 1H), 8.73-8.67 (m, 1H), 8.06-7.98 (m, 1H), 7.93-7.87 (m, 1H), 7.82-7.73 (m, 1H), 7.72-7.63 (m, 2H), 7.60-7.33 (m, 5H), 7.16-7.01 (m, 1H), 6.95-6.55 (m, 2H), 6.49-5.88 (m, 2H), 5.88-5.22 (m, 1H), 4.99-4.43 (m, 3H), 4.36-4.14 (m, 2H), 3.74-3.39 (m, 1H), 3.24-2.99 (m, 2H), 2.37-2.29 (m, 3H), 2.02-1.14 (m, 9H).

### Example 194—Synthesis of Compound 194

### Synthetic Scheme

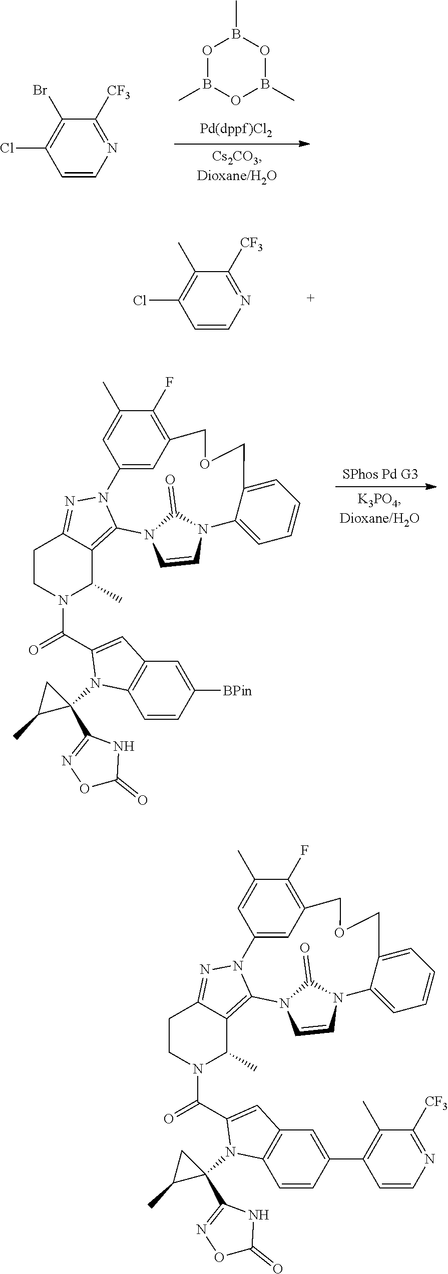

### Procedure for Synthesis of Compound 194

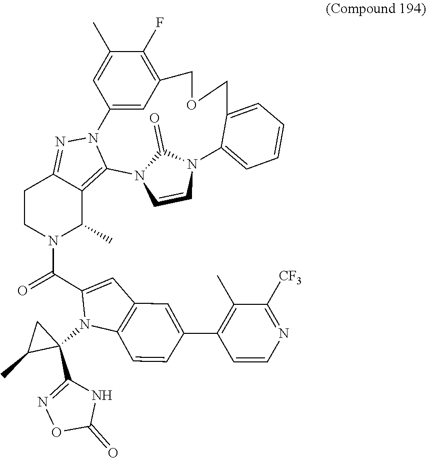

### (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-[3-methyl-2-(trifluoromethyl)-4-pyridyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of 3-bromo-4-chloro-2-(trifluoromethyl)pyridine (0.1 g, 384 μmol) in 1,4-dioxane (2 mL) and water (0.5 mL) was added trimethylboroxin (96.4 mg, 768 μmol), dicaesium carbonate (188 mg, 576 μmol), and Pd(dppf)Cl₂ (15.7 mg, 19.2 μmol). The reaction was heated at 100° C. for 1 hour, then quenched with water and extracted with EtOAc. The organic extract was dried over Na₂SO₄, filtered, and concentrated under reduced pressure. The residue was purified by silica gel column chromatography eluting with 0-10% EtOAc/heptane to afford 4-chloro-3-methyl-2-(trifluoromethyl)pyridine (12.8 mg, 65.4 μmol). m/z (ESI-pos) M+1=196.0

Step B: To a 4 mL vial was added (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-(4,4,5,5-tetramethyl-1,3,2-dioxaborolan-2-yl)-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (16 mg, 18.8 μmol), 4-chloro-3-methyl-2-(trifluoromethyl)pyridine (5.5 mg, 28.1 μmol), 1,4-dioxane (250 μL), tripotassium phosphate (7.97 mg, 37.5 μmol), water (125 μL), and SPhos Pd G3 (733 μg, 0.938 μmol). The mixture was sparged with nitrogen for 5 minutes, then heated to 90° C. for 3 hours. The reaction was partitioned between ethyl acetate and water/brine. The organics were collected, dried over sodium sulfate, filtered and concentrated in vacuo. The crude residue was purified by reverse-phase prep-HPLC purification eluting with 40-100% MeCN/water with 0.1% formic acid, desired fractions were lyophilized to afford (8S,10M)-25-fluoro-8,26-dimethyl-7-({1-[(1S,2S)-2-methyl-1-(5-oxo-4,5-dihydro-1,2,4-oxadiazol-3-yl)cyclopropyl]-5-[3-methyl-2-(trifluoromethyl)-4-pyridyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (8.37 mg, 9.16 μmol). m/z (ESI-pos) M+1=886.5; ¹H NMR (400 MHz, CHLOROFORM-D) δ 11.46-11.07 (m, 1H), 8.59-8.53 (m, 1H), 7.78-7.27 (m, 9H), 7.15-7.01 (m, 1H), 6.90-6.76 (m, 1H), 6.48-5.89 (m, 2H), 5.88-5.25 (m, 1H), 5.00-4.08 (m, 5H), 3.80-3.02 (m, 3H), 2.45-2.38 (m, 3H), 2.37-2.31 (m, 3H), 2.11-1.40 (m, 6H), 1.25 (m, 3H).

### Example 195—Synthesis of Compound 195

### Procedure for Synthesis of Compound 195

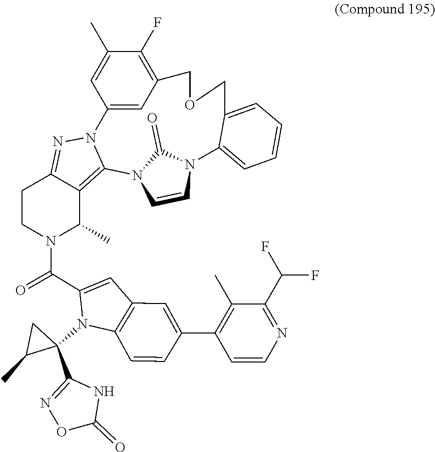

### (8S,10M)-7-({5-[2-(difluoromethyl)-3-methyl-4-pyridyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 195 was synthesized following the procedure of Example 194, with 4-chloro-3-methyl-2-(trifluoromethyl)pyridine being replaced with 4-bromo-2-(difluoromethyl)-3-methylpyridine. m/z (ESI-pos) M+1=868.6; ¹H NMR (400 MHz, CHLOROFORM-D) δ 11.36-11.23 (m, 1H), 8.52-8.46 (m, 1H), 7.78-7.27 (m, 9H), 7.16-6.64 (m, 3H), 6.49-5.91 (m, 2H), 5.88-5.25 (m, 1H), 4.99-4.12 (m, 5H), 3.74-3.38 (m, 1H), 3.23-2.98 (m, 2H), 2.46-2.41 (m, 3H), 2.37-2.32 (m, 3H), 2.02-1.15 (m, 9H).

### Example 196—Synthesis of Compound 196

### Procedure for Synthesis of Compound 196

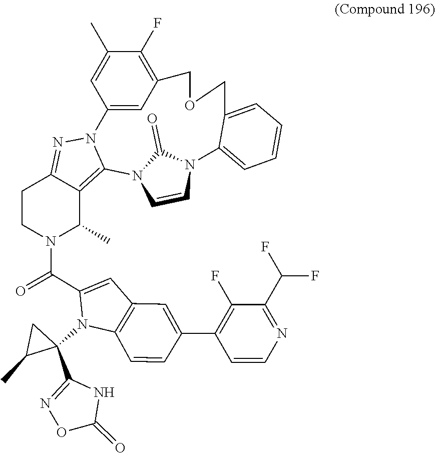

### (8S,10M)-7-({5-[2-(difluoromethyl)-3-fluoro-4-pyridyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 196 was synthesized following the procedure of Example 194, with 4-chloro-3-methyl-2-(trifluoromethyl)pyridine being replaced with 4-bromo-2-(difluoromethyl)-3-fluoropyridine. m/z (ESI-pos) M+1=872.5; ¹H NMR (400 MHz, CHLOROFORM-D) δ 11.32-11.19 (m, 1H), 8.56-8.50 (m, 1H), 8.02-7.94 (m, 1H), 7.79 (m, 1H), 7.71-7.33 (m, 7H), 7.15-6.72 (m, 3H), 6.48-5.89 (m, 2H), 5.88-5.23 (m, 1H), 4.99-4.10 (m, 5H), 3.74-3.39 (m, 1H), 3.24-3.03 (m, 2H), 2.37-2.30 (m, 3H), 2.04-1.14 (m, 9H).

### Example 197—Synthesis of Compound 197

### Synthetic Scheme

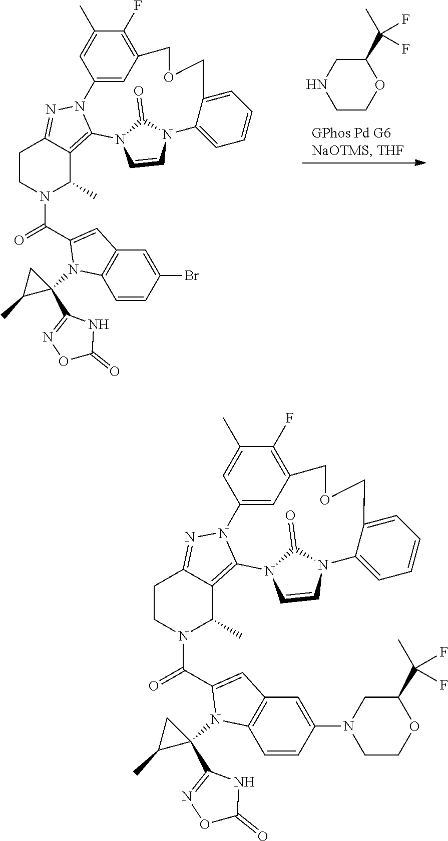

### Procedure for Synthesis of Compound 197

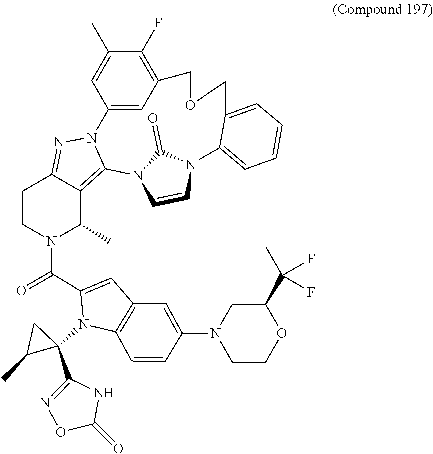

### (8S,10M)-7-({5-[(S)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: A 4 mL vial was charged with (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (30 mg, 37.2 μmol), (S)-2-(1,1-difluoroethyl) morpholine (5.63 mg, 37.2 μmol), GPhos Pd G6 (3.52 mg, 3.72 μmol), sodium trimethylsilanolate (16.7 mg, 149 μmol), followed by addition of tetrahydrofuran (931 μL). The mixture was sparged with nitrogen for 5 minutes then heated to 70° C. After 16 hours, the reaction was cooled and partitioned between saturated aqueous ammonium chloride and ethyl acetate. The aqueous was extracted twice with ethyl acetate, then the organics were dried over sodium sulfate, filtered, and concentrated in vacuo. The crude residue was purified by ACCQ reverse phase prep-HPLC eluting with 40-100% MeCN+0.1% formic acid in water+0.1% formic acid. The desired fractions were lyophilized overnight to afford (8S,10M)-7-({5-[(S)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (10.9 mg, 12.5 μmol). m/z (ESI-pos) M+1=876.6; ¹H NMR (400 MHz, CHLOROFORM-D) δ 11.32 (s, 1H), 7.61-7.34 (m, 6H), 7.22-7.01 (m, 3H), 6.77-6.64 (m, 1H), 6.48-5.86 (m, 2H), 5.53 (m, 1H), 4.95-2.73 (m, 15H), 2.37-1.10 (m, 14H).

### Example 198—Synthesis of Compound 198

### Synthetic Scheme

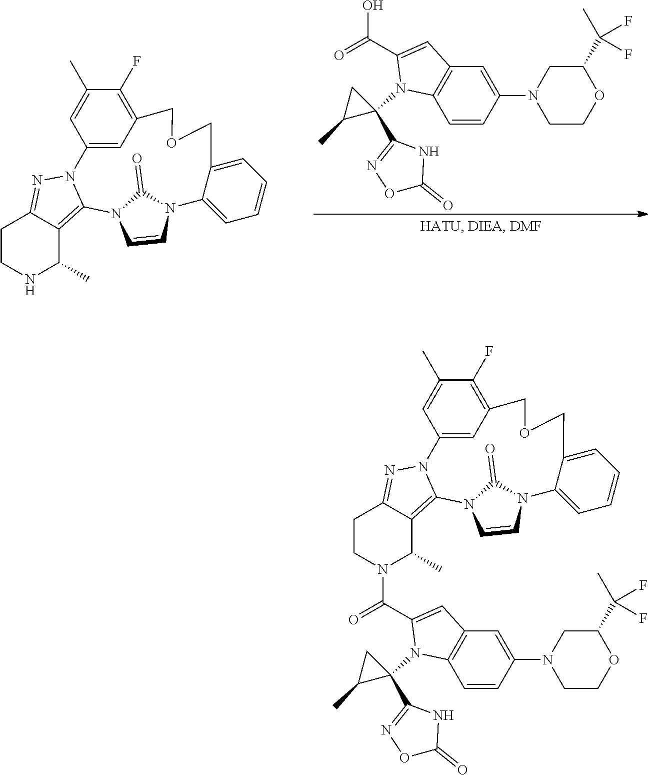

### Procedure for Synthesis of Compound 198

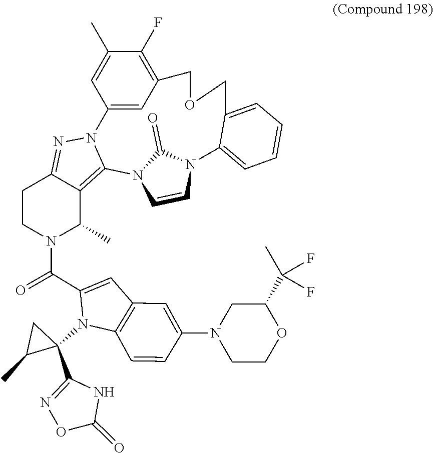

### (8S,10M)-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (8S,10M)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (221 mg, 459 μmol) and 5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolecarboxylic acid (226 mg, 504 μmol) in dimethylformamide (1.83 mL) was added HATU (262 mg, 688 μmol) and N-ethyldiisopropylamine (319 μL, 1.83 mmol). The mixture stirred at room temperature. After 16 hours, the reaction was diluted with EtOAc, and washed with saturated aqueous NH₄Cl and brine, extracted three times with EtOAC, and concentrated. The crude residue was dissolved in MeCN/MeOH/H₂O and loaded onto the reverse phase column and purified by prep-HPLC purification eluting with 20-80% MeCN/H₂O with 0.1% formic acid buffer. Combined pure fractions lyophilized overnight to afford (8S,10M)-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (325 mg, 371 μmol). m/z (ESI-pos) M+1=876.6; ¹H NMR (400 MHz, CHLOROFORM-D) δ 11.41-11.22 (m, 1H), 7.61-7.33 (m, 5H), 7.21-6.97 (m, 3H), 6.77-6.64 (m, 1H), 6.48-5.85 (m, 2H), 5.85-5.21 (m, 1H), 4.95-2.74 (m, 15H), 2.37-1.10 (m, 14H).

### Example 199—Synthesis of Compound 199

### Synthetic Scheme

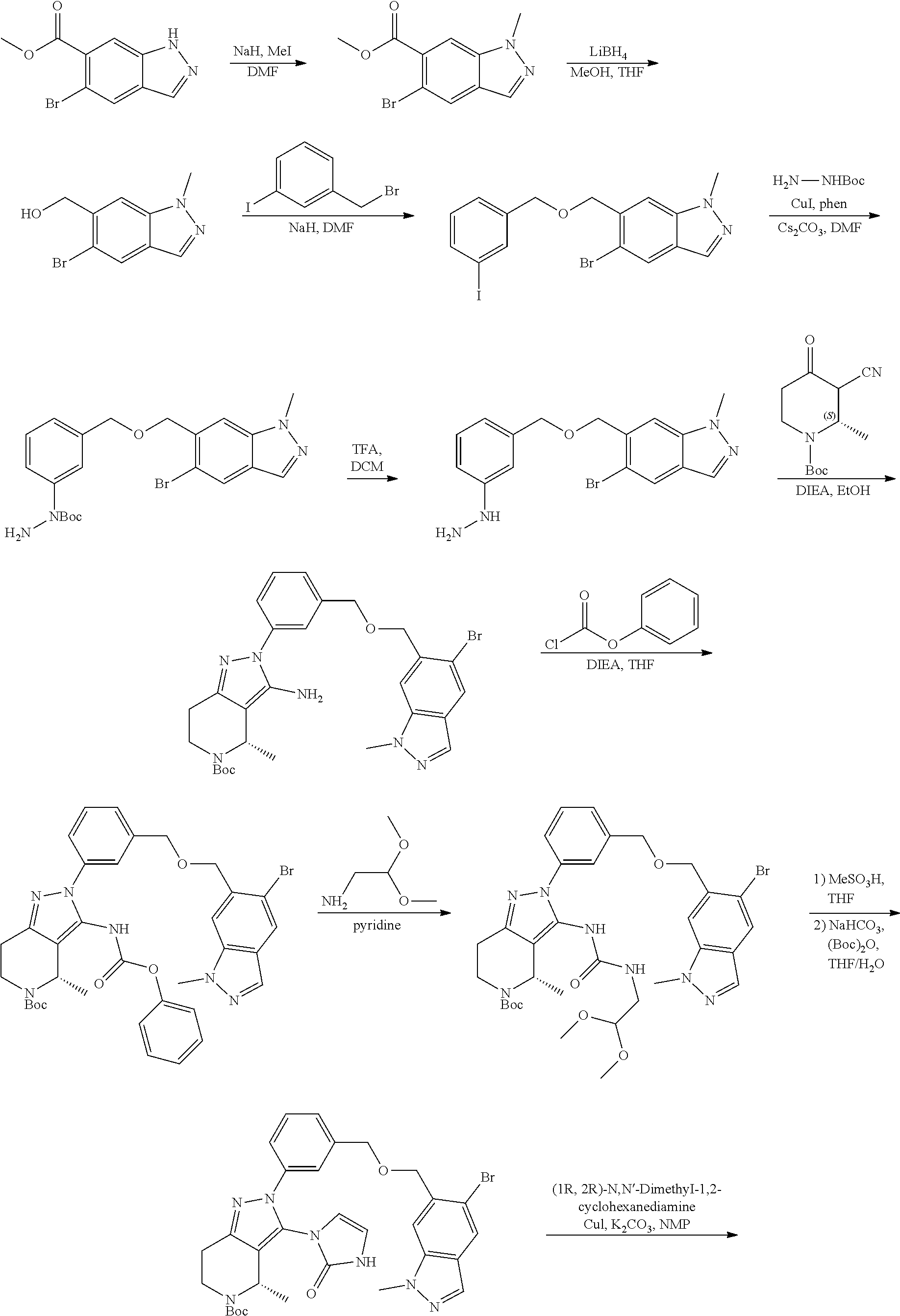

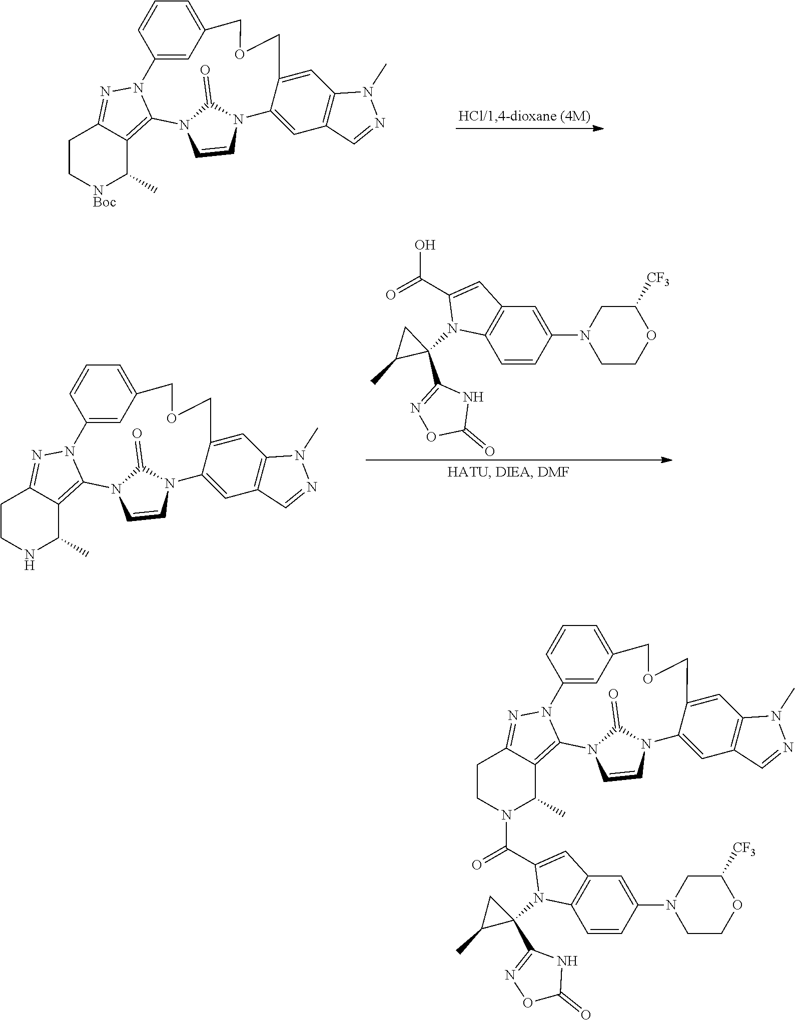

### Procedure for Synthesis of Compound 199

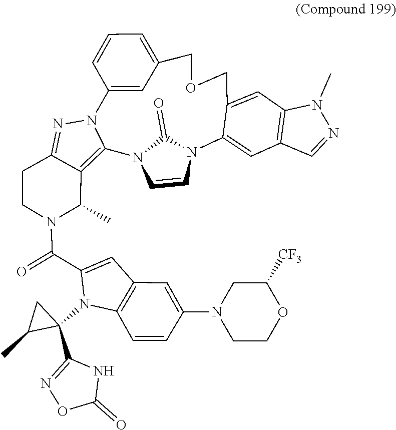

### (8S,10M)-8-methyl-20-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-25-oxa-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21), 18,22,27,29-decaen-32-one

Step A: To the solution of methyl 5-bromo-1H-indazole-6-carboxylate (5 g, 19.6 mmol) in dimethylformamide (39.2 mL) was added 60% sodium hydride in mineral oil (941 mg, 23.5 mmol) at 0° C. under nitrogen. The solution was stirred at 0° C. for 5 minutes, then iodomethane (2.44 mL, 39.2 mmol) was added in one portion and reaction was removed from the ice bath and stirred at room temperature for 16 hours. The reaction mixture was quenched with addition of saturated aqueous NaHCO₃, diluted with ethyl acetate, and the organics were washed twice with brine. The organic layer was dried over Na₂SO₄ and concentrated in vacuo. The crude product was purified via column chromatography eluting with 0-55% EtOAc/Heptane afford methyl 5-bromo-1-methyl-1H-indazole-6-carboxylate (3.1 g, 11.5 mmol) as the first eluting isomer peak. m/z (ESI-pos) M+1=269.0/271.0

Step B: LiBH₄ (374 mg, 17.2 mmol) was added to a solution of methyl 5-bromo-1-methyl-1H-indazole-6-carboxylate (3.08 g, 11.4 mmol) in tetrahydrofuran (11.4 mL) and methanol (695 μL) at 0° C. under nitrogen. The reaction was heated to 55° C. for 16 hours. Reaction was quenched with addition of water then extracted with EtOAc twice. Organics washed with brine, dried over sodium sulfate, filtered, and concentrated in vacuo. The crude residue was purified via column chromatography eluting with 0-6% MeOH/DCM to afford (5-bromo-1-methyl-1H-indazol-6-yl)methanol (1.15 g, 4.77 mmol). m/z (ESI-pos) M+1=241.0/243.0

Step C: To the solution of (5-bromo-1-methyl-1H-indazol-6-yl)methanol (0.6 g, 2.49 mmol) in dimethylformamide (8.3 mL) was added 60% sodium hydride in mineral oil (149 mg, 3.73 mmol) at 0° C. under nitrogen. The solution was stirred at 0° C. for 5 minutes, then m-(bromomethyl) iodobenzene (887 mg, 2.99 mmol) was added in one portion and the reaction was removed from the ice bath and stirred at room temperature for 1 hour. The reaction mixture was quenched with saturated aqueous NaHCO₃, diluted with ethyl acetate, and washed with brine twice. The organic layer was dried over Na₂SO₄ and concentrated in vacuo. The crude product was purified by column chromatography eluting with 0-30% ethyl acetate/heptane to afford 5-bromo-6-{[(m-iodophenyl)methoxy]methyl}-1-methyl-1H-indazole (1.07 g, 2.34 mmol). m/z (ESI-pos) M+1=457.0/459.1

Step D: A solution of 5-bromo-6-{[(m-iodophenyl)methoxy]methyl}-1-methyl-1H-indazole (1.07 g, 2.34 mmol), tert-butyl carbazate (402 mg, 3.04 mmol), dicaesium carbonate (1.07 g, 3.28 mmol), copper iodide (22.3 mg, 117 μmol), and 4,5-diazaphenanthrene (84.4 mg, 468 μmol) in dimethylformamide (2.34 mL) was sparged with nitrogen for 5 minutes and heated to 80° C. for 16 hours. The solution was taken up in EtOAc, filtered, and washed with brine three times. The organics were dried over Na₂SO₄, filtered, and concentrated. The crude product purified by silica gel chromatography eluting with 5-40% EtOAc/heptane to afford tert-butyl 2-(m-{[(5-bromo-1-methyl-1H-indazol-6-yl)methoxy]methyl}phenyl)carbazate (790 mg, 1.71 mmol). m/z (ESI-pos) M+1=405.1/407.1 (-tBu)

Step E: Trifluoroacetic acid (2.62 mL, 34.2 mmol) was added to a solution of tert-butyl 2-(m-{[(5-bromo-1-methyl-1H-indazol-6-yl)methoxy]methyl}phenyl)carbazate (790 mg, 1.71 mmol) in dichloromethane (5 mL). The mixture was stirred at room temperature for 2.5 hours. The mixture was quenched with addition of 2M aqueous Na₂CO₃ and the aqueous was extracted with DCM. Organics washed again with 2M aqueous Na₂CO₃ solution, dried over magnesium sulfate, filtered and concentrated in vacuo to afford (m-{[(5-bromo-1-methyl-1H-indazol-6-yl)methoxy]methyl}phenyl)hydrazine (0.6 g, 1.66 mmol). m/z (ESI-pos) M+1=361.1/363.1

Step F: To a solution of (m-{[(5-bromo-1-methyl-1H-indazol-6-yl)methoxy]methyl}phenyl)hydrazine (0.6 g, 1.66 mmol) in ethanol (5.54 mL) was added N-ethyldiisopropylamine (434 μL, 2.49 mmol) then tert-butyl(2S)-3-cyano-2-methyl-4-oxo-1-piperidinecarboxylate (475 mg, 1.99 mmol) at room temperature. The mixture was stirred at 80° C. After 16 hours, the reaction mixture was then cooled down to room temperature and concentrated. The residue was taken up in water and extracted with EtOAc twice. The organic layers were dried over anhydrous Na₂SO₄ and concentrated in vacuo. The crude compound was purified by column chromatography eluting with 10-95% EtOAc/heptane to afford tert-butyl (S)-3-amino-2-(m-{[(5-bromo-1-methyl-1H-indazol-6-yl)methoxy]methyl}phenyl)-4-methyl-1,2,5-triaza-2,4,6,7-tetrahydro-5H-indene-5-carboxylate (420 mg, 722 μmol). m/z (ESI-pos) M+1=581.3/583.3

Step G: To a solution of tert-butyl (S)-3-amino-2-(m-{[(5-bromo-1-methyl-1H-indazol-6-yl)methoxy]methyl}phenyl)-4-methyl-1,2,5-triaza-2,4,6,7-tetrahydro-5H-indene-5-carboxylate (380 mg, 653 μmol) in tetrahydrofuran (4.36 mL) was added N-ethyldiisopropylamine (228 μL, 1.31 mmol) followed by benzenyl chloroformate (123 μL, 980 μmol). The reaction mixture was stirred at 25° C. for 1 hour. The reaction mixture was directly concentrated under reduced pressure to give the crude tert-butyl (S)-2-(m-{[(5-bromo-1-methyl-1H-indazol-6-yl)methoxy]methyl}phenyl)-4-methyl-3-[phenyl(oxycarbonylamino)]-1,2,5-triaza-2,4,6,7-tetrahydro-5H-indene-5-carboxylate (458 mg, 653 μmol) as a yellow oil, which was used directly in the next step without any further purification. m/z (ESI-pos) M+1=701.3/703.4

Step H: Crude tert-butyl (S)-2-(m-{[(5-bromo-1-methyl-1H-indazol-6-yl)methoxy]methyl}phenyl)-4-methyl-3-[phenyl(oxycarbonylamino)]-1,2,5-triaza-2,4,6,7-tetrahydro-5H-indene-5-carboxylate (458 mg, 653 μmol) was dissolved in pyridine (3.26 mL) and then 2,2-dimethoxyethylamine (213 μL, 1.96 mmol) was added. The mixture was stirred at room temperature for 1 hour and then directly concentrated under reduced pressure. The crude residue was purified by silica gel column chromatography eluting with 10-100% EtOAc in Heptane to afford tert-butyl (S)-2-(m-{[(5-bromo-1-methyl-1H-indazol-6-yl)methoxy]methyl}phenyl)-3-[3-(2,2-dimethoxyethyl)ureido]-4-methyl-1,2,5-triaza-2,4,6,7-tetrahydro-5H-indene-5-carboxylate (390 mg, 547 μmol). m/z (ESI-pos) M+1=712.4/714.4

Step I: To a solution of tert-butyl (S)-2-(m-{[(5-bromo-1-methyl-1H-indazol-6-yl)methoxy]methyl}phenyl)-3-[3-(2,2-dimethoxyethyl)ureido]-4-methyl-1,2,5-triaza-2,4,6,7-tetrahydro-5H-indene-5-carboxylate (390 mg, 547 μmol) in tetrahydrofuran (5.47 mL) was added methanesulfonic acid (178 μL, 2.74 mmol). The mixture was stirred at 60° C. for 30 minutes. Aqueous saturated sodium bicarbonate was added to adjusted pH to about 8, and then tert-butyl-tert-butyl(oxycarbonyloxy)formylate (179 mg, 821 μmol) was added, and the resulting mixture was stirred at room temperature for 30 minutes. The reaction mixture was diluted with ethyl acetate and washed with water twice. The organic layer was then dried over anhydrous Na₂SO₄, filtered and the filtrate was evaporated under reduced pressure. The crude residue was purified via column chromatography eluting with 10-100% EtOAc/Heptane to afford tert-butyl (S)-2-(m-{[(5-bromo-1-methyl-1H-indazol-6-yl)methoxy]methyl}phenyl)-4-methyl-3-(2-oxo-2,3-dihydro-1H-imidazol-1-yl)-1,2,5-triaza-2,4,6,7-tetrahydro-5H-indene-5-carboxylate (307 mg, 473 μmol). m/z (ESI-pos) M+1=592.3/594.3 (-tBu)

Step J: Tert-butyl (S)-2-(m-{[(5-bromo-1-methyl-1H-indazol-6-yl)methoxy]methyl}phenyl)-4-methyl-3-(2-oxo-2,3-dihydro-1H-imidazol-1-yl)-1,2,5-triaza-2,4,6,7-tetrahydro-5H-indene-5-carboxylate (0.2 g, 308 μmol), powdered dipotassium carbonate (128 mg, 925 μmol), copper iodide (44 mg, 231 μmol), 1,8-diphenyl-4,5-diazaphenanthrene (103 mg, 308 μmol) and dimethyl sulfoxide (12.3 mL) were added to a vial and sparged with nitrogen for 10 minutes. The solution was stirred at 130° C. for 42 hours. The reaction was cooled to room temperature, and water, aqueous saturated ammonium chloride and EtOAc were added to the stirring solution. The mixture was filtered over a disposable filter funnel, washing with EtOAc and transferred to a separatory funnel and organics were collected. The aqueous layer was extracted again with EtOAc. Combined organics were washed with ~4:1 water/saturated ammonium chloride, then brine. The organics were then dried over magnesium sulfate, filtered and concentrated in vacuo. Crude material was purified via column chromatography eluting with 20-80% EtOAc/Heptane. Mixed fractions were repurified by ACCQ prep-HPLC eluting with 40-60% MeCN+0.1% formic acid in water+0.1% formic acid, desired fractions concentrated in vacuo to afford tert-butyl (8S,10M)-8-methyl-20-methyl-32-oxo-25-oxa-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(30),3,9,12,15 (23),16,18,21,27 (31),28-decaene-7-carboxylate (32.3 mg, 56.8 μmol). m/z (ESI-pos) M+1=568.4

Step K: 4M HCl in Dioxane (0.5 mL) was added to a stirring solution of tert-butyl (8S,10M)-8-methyl-20-methyl-32-oxo-25-oxa-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(30),3,9,12,15 (23),16,18,21,27 (31),28-decaene-7-carboxylate (5.25 mg, 9.25 μmol) in dichloromethane (360 μL). The mixture was stirred at room temperature for 3 hours. The mixture was concentrated in vacuo to afford (8S,10M)-8-methyl-20-methyl-25-oxa-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(30),3,9,12,15 (23),16,18,21,27 (31),28-decaen-32-one-hydrogen chloride (1/1) (4.66 mg, 9.25 μmol). m/z (ESI-pos) M+1=468.4

Step L: An 8 mL vial was charged with (8S,10M)-8-methyl-20-methyl-25-oxa-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21),18,22,27,29-decaen-32-one-hydrogen chloride (1/1) (5 mg, 9.92 μmol), dimethylformamide (496 μL), and N-ethyldiisopropylamine (8.64 μL, 49.6 μmol). 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid (5.39 mg, 11.9 μmol) was then added followed by [(dimethylamino)(3-hydroxy-1λ⁵,2,3,4-tetraaza-3H-inden-1-yl)methylene]bis(methyl)azanium hexafluoridophosphate (1-) (5.67 mg, 14.9 μmol) and the mixture was stirred at room temperature for 16 hours. Reaction was transferred with MeCN/H₂O and purified directly by reverse phase prep-HPLC purification eluting with 40-100% MeCN+0.1% formic acid in H₂O+0.1% formic acid. Desired fractions were dried in vacuo to afford (8S,10M)-8-methyl-20-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-25-oxa-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21),18,22,27,29-decaen-32-one (4.45 mg, 4.72 μmol). m/z (ESI-pos) M+1=902.7; ¹H NMR (400 MHz, CHLOROFORM-D) δ 11.36-11.25 (m, 1H), 8.07-7.08 (m, 10H), 6.73 (d, J=35.2 Hz, 1H), 6.50-5.88 (m, 2H), 5.88-5.24 (m, 1H), 4.79-2.83 (m, 18H), 2.02-1.11 (m, 9H).

### Example 200—Synthesis of Compound 200

### Procedure for Synthesis of Compound 200

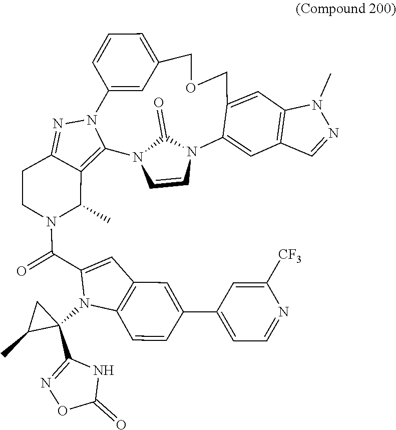

### (8S,10M)-8-methyl-20-methyl-7-({1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolyl}carbonyl)-25-oxa-2,3,7,11,14,19,20-heptaazaheptacyclo[25.3.1.1¹¹,¹⁴.0²,¹⁰.0⁴,⁹.0¹⁵,²³.0¹⁷,²¹]dotriaconta-1(31),3,9,12,15,17(21), 18,22,27,29-decaen-32-one

Compound 200 was synthesized following the procedure of Example 199, with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid being replaced with 1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolecarboxylic acid. m/z (ESI-pos) M+1=894.5; ¹H NMR (400 MHz, CHLOROFORM-D) δ 11.31-11.20 (m, 1H), 8.81-8.74 (m, 1H), 8.07-7.18 (m, 12H), 6.97-6.83 (m, 1H), 6.49-5.90 (m, 2H), 5.90-5.25 (m, 1H), 4.99-3.05 (m, 11H), 2.04-1.15 (m, 9H).

### Example 201—Synthesis of Compound 201

### Synthetic Scheme

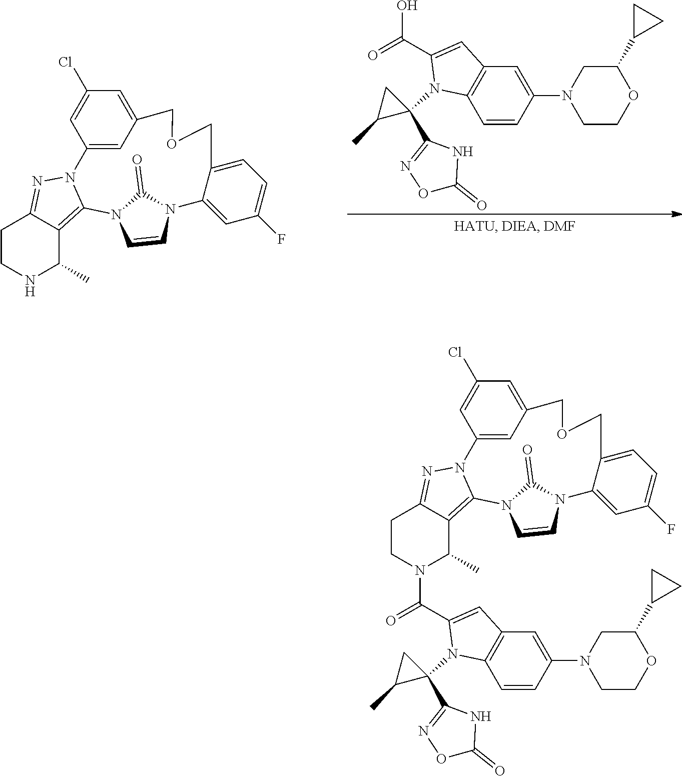

### Procedure for Synthesis of Compound 201

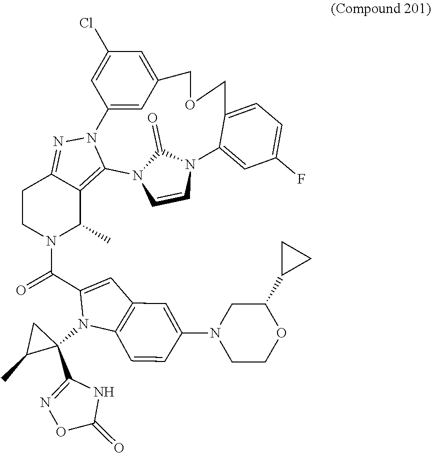

### (8S,10M)-26-chloro-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (8S,10M)-26-chloro-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (17.5 mg, 34.8 μmol) and 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid (16.3 mg, 38.3 μmol) in dimethylformamide (348 μL) was added N-ethyldiisopropylamine (24.3 μL, 139 μmol) and HATU (17.2 mg, 45.3 μmol). The mixture was stirred at room temperature. After 16 hours, the reaction mixture was directly purified by prep-HPLC eluting with 40 to 100% MeCN/H₂O with 0.1% formic acid buffer, pure fractions were collected and lyophilized to afford (8S,10M)-26-chloro-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (11.8 mg, 13.5 μmol). m/z (ESI-pos) M+1=872.6; ¹H NMR (400 MHz, CHLOROFORM-D) δ 11.27 (dm, J=24.6 Hz, 1H), 7.75-7.45 (m, 3H), 7.22-6.97 (m, 4H), 6.77-6.64 (m, 1H), 6.55-5.91 (m, 2H), 5.86-5.25 (m, 1H), 4.97-2.66 (m, 15H), 2.03-0.25 (m, 14H).

### Example 202—Synthesis of Compound 202

### Synthetic Scheme

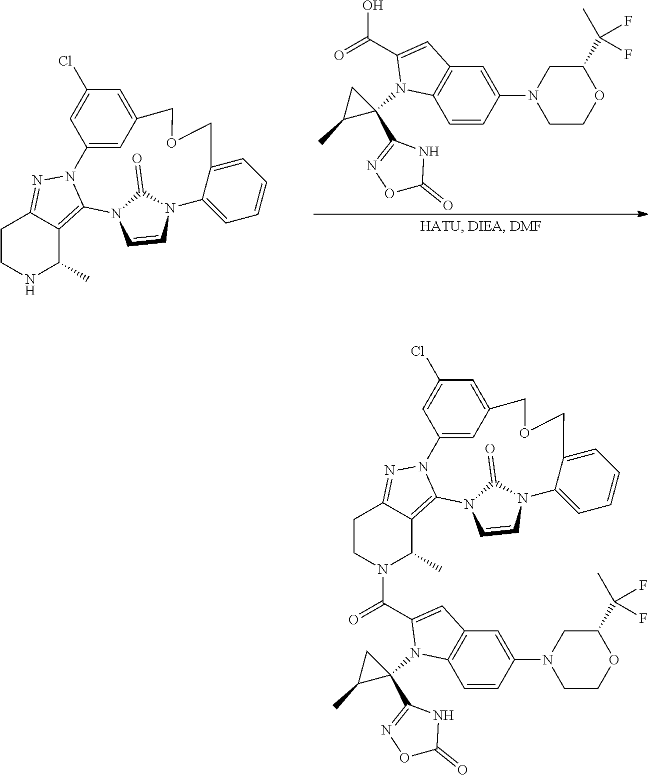

### Procedure for Synthesis of Compound 202

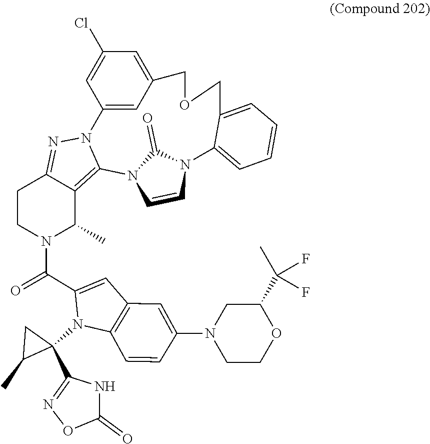

### (8S,10M)-26-chloro-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (8S,10M)-26-chloro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (14.1 mg, 29.1 μmol) and 5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolecarboxylic acid (14.4 mg, 32 μmol) in dimethylformamide (291 μL) was added N-ethyldiisopropylamine (20.3 μL, 116 μmol) and HATU (14.4 mg, 37.8 μmol). The mixture stirred at room temperature for 16 hours. The reaction mixture was directly purified by prep-HPLC eluting with 40-100% MeCN/H₂O with 0.1% formic acid buffer, pure fractions were collected and lyophilized to afford (8S,10M)-26-chloro-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (14.1 mg, 16.1 μmol). m/z (ESI-pos) M+1=878.5; ¹H NMR (400 MHz, CHLOROFORM-D) δ 11.31 (m, 1H), 7.75-7.31 (m, 6H), 7.22-7.02 (m, 4H), 6.77-6.65 (m, 1H), 6.55-5.90 (m, 2H), 5.86-5.23 (m, 1H), 4.97-2.73 (m, 15H), 1.96-1.09 (m, 12H).

### Example 203—Synthesis of Compound 203

### Synthetic Scheme

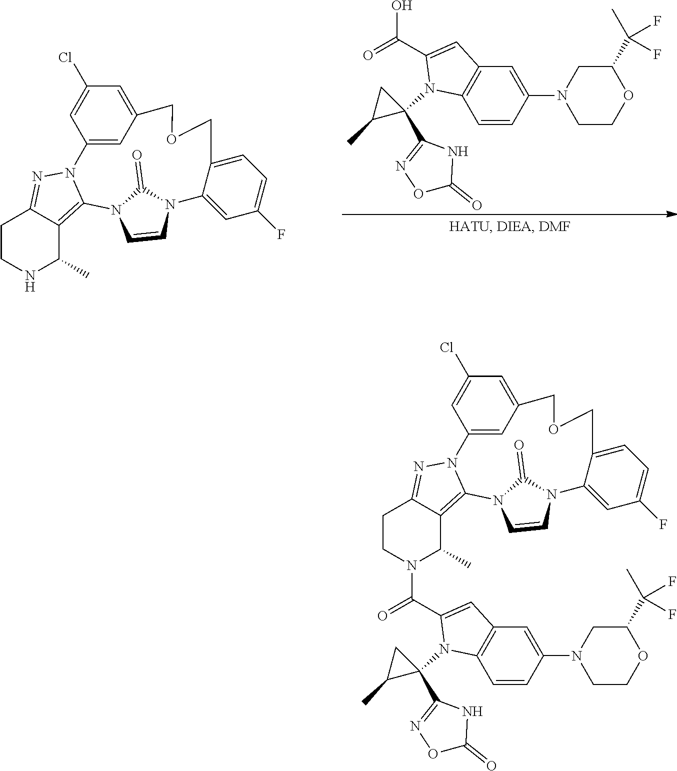

### Procedure for Synthesis of Compound 203

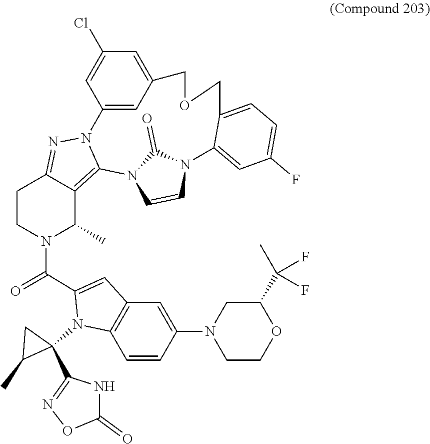

### (8S,10M)-26-chloro-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (8S,10M)-26-chloro-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (16.9 mg, 33.6 μmol) and 5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolecarboxylic acid (16.6 mg, 37 μmol) in dimethylformamide (336 μL) was added N-ethyldiisopropylamine (23.4 μL, 135 μmol) and HATU (16.6 mg, 43.7 μmol). The mixture stirred at room temperature for 16 hours. The reaction mixture was directly purified by prep-HPLC eluting with 40-100% MeCN/H₂O with 0.1% formic acid buffer, pure fractions were collected and lyophilized to afford (8S,10M)-26-chloro-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (18.3 mg, 20.4 μmol). m/z (ESI-pos) M+1=896.5; ¹H NMR (400 MHz, CHLOROFORM-D) δ 11.32-11.21 (m, 1H), 7.75-7.44 (m, 3H), 7.22-6.98 (m, 6H), 6.77-6.65 (m, 1H), 6.54-5.92 (m, 2H), 5.86-5.24 (m, 1H), 4.97-2.71 (m, 14H), 1.94-1.11 (m, 13H).

### Example 204—Synthesis of Compound 204

### Procedure for Synthesis of Compound 204

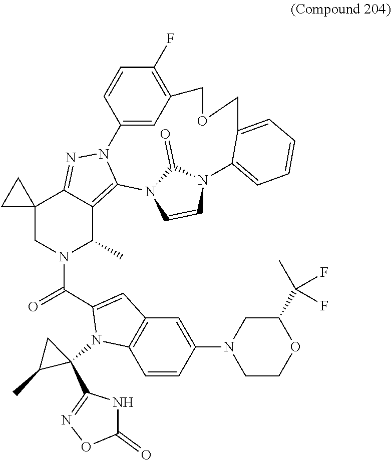

### (8S,10M)-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one

Compound 204 was synthesized following the procedure of Example 74, with tert-butyl (2S)-3-cyano-2-methyl-4-oxo-1-piperidinecarboxylate being replaced with tert-butyl (6S)-7-cyano-6-methyl-8-oxo-5-aza-5-spiro[2.5]octanecarboxylate. m/z (ESI-pos) M+1=888.7; ¹H NMR (400 MHz, CHLOROFORM-D) δ 11.35-11.28 (m, 1H), 7.62-7.34 (m, 6H), 7.29-7.08 (m, 4H), 6.79-6.66 (m, 1H), 6.46-6.23 (m, 2H), 5.92-5.32 (m, 1H), 4.81-2.72 (m, 14H), 1.95-0.77 (m, 16H).

### Example 205—Synthesis of Compound 205

### Procedure for Synthesis of Compound 205

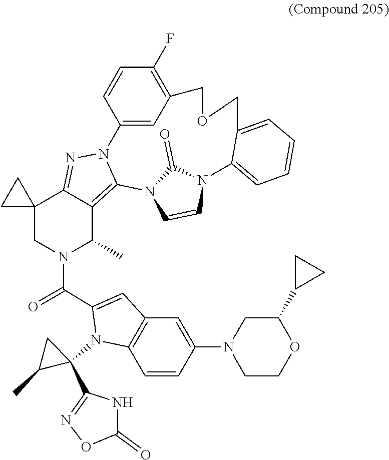

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25-fluoro-8-methylspiro[(M)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-5,1′-cyclopropan]-29-one

Compound 205 was synthesized following the procedure of Example 73, with tert-butyl (2S)-3-cyano-2-methyl-4-oxo-1-piperidinecarboxylate being replaced with tert-butyl (6S)-7-cyano-6-methyl-8-oxo-5-aza-5-spiro[2.5]octanecarboxylate. m/z (ESI-pos) M+1=864.7; ¹H NMR (400 MHz, CHLOROFORM-D) δ 11.33 (m, 1H), 7.69-7.34 (m, 7H), 7.29-7.10 (m, 3H), 6.78-6.66 (m, 1H), 6.46-6.23 (m, 2H), 5.93-5.33 (m, 1H), 4.81-2.65 (m, 12H), 1.96-0.24 (m, 16H).

### Example 206—Synthesis of Compound 206

### Synthetic Scheme

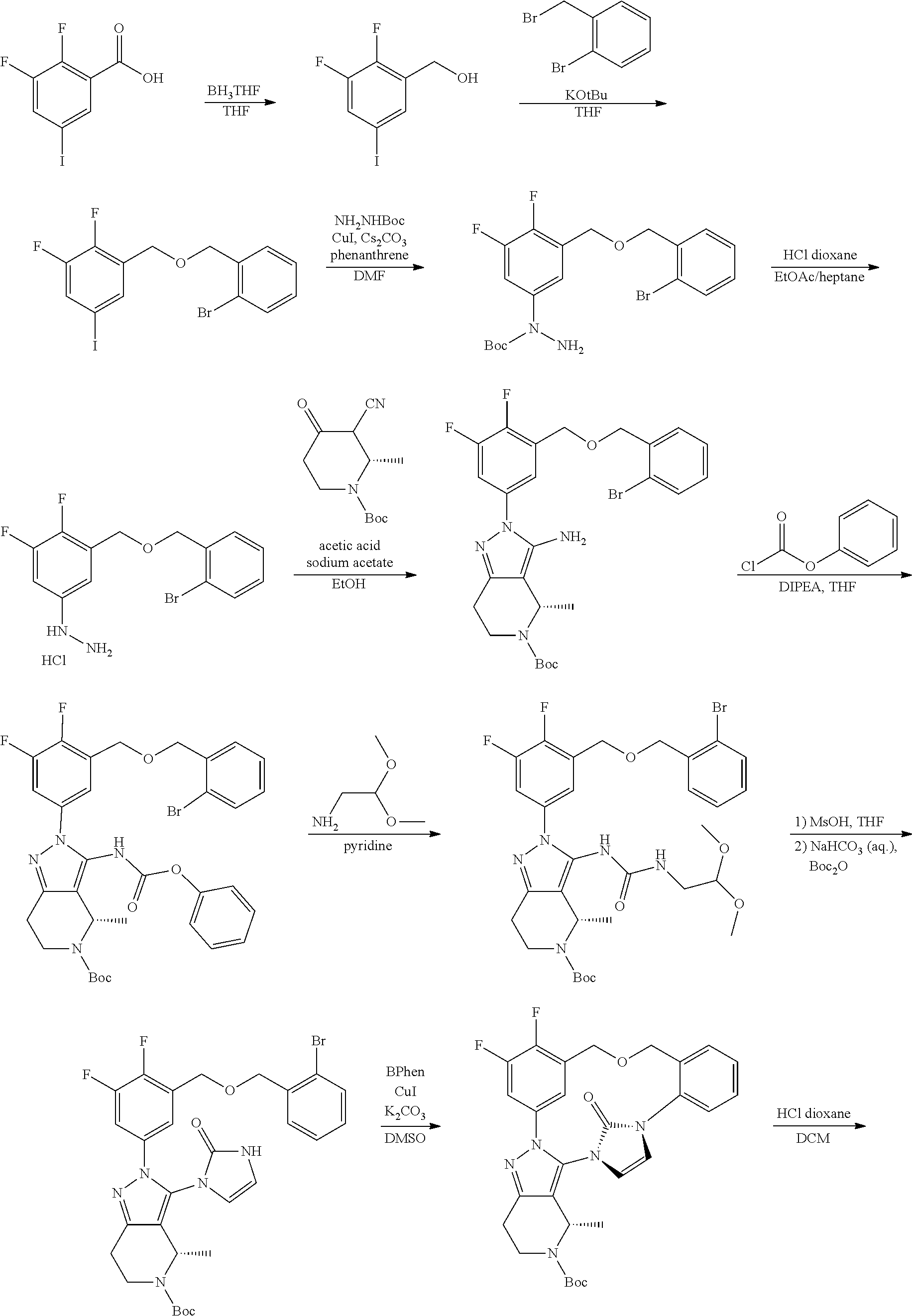

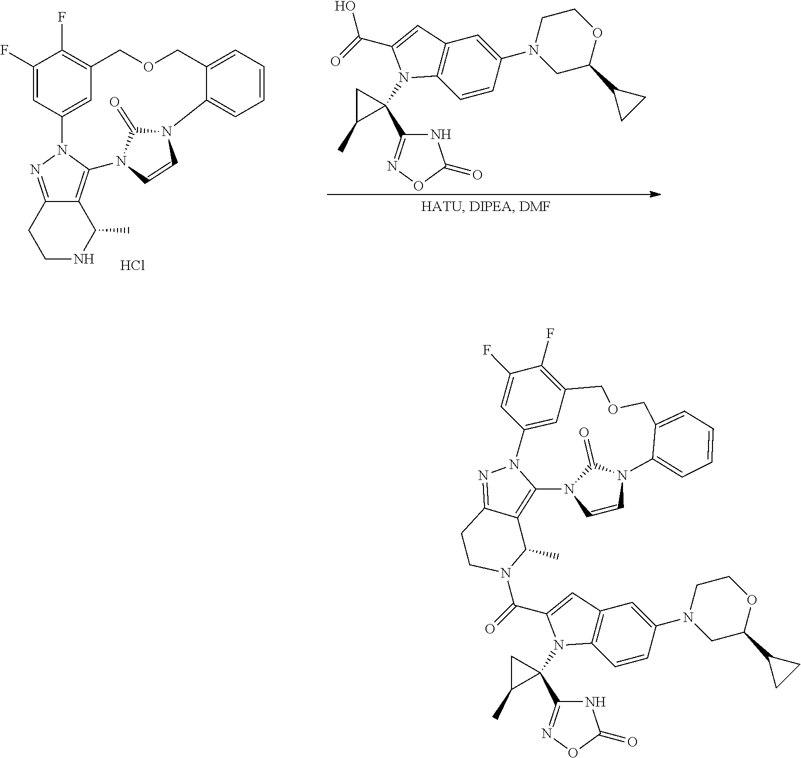

### Procedure for Synthesis of Compound 206

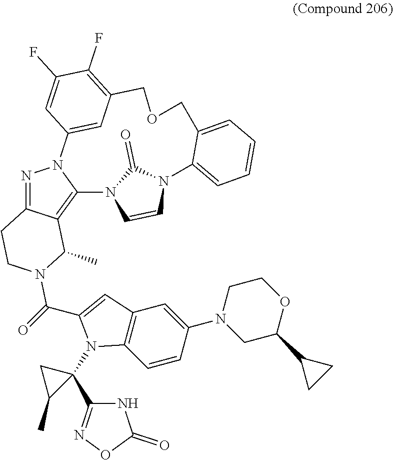

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25,26-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: 1M Borane in THF (70.4 mL, 70.4 mmol) was added dropwise to a stirred solution of 2,3-difluoro-5-iodobenzoic acid (10 g, 35.2 mmol) in THF (88 mL) at 0° C. The reaction was stirred at 0° C. for 30 minutes and at room temperature for 2 hours. Additional 1M Borane in THF (20 mL, 20 mmol) was added at room temperature and the reaction was stirred for 1 hour. The reaction mixture was cooled to 0° C., slowly quenched with saturated aqueous NaHCO₃ and extracted with EtOAc. The organic layer was washed with brine (2×), dried over Na₂SO₄, and concentrated in vacuo to afford (2,3-difluoro-5-iodophenyl)methanol (9.15 g, 33.9 mmol) which was used in the next step without purification.

Step B: Potassium 2-methyl-2-propanolate (4.94 g, 44.1 mmol) was added to a stirred solution of (2,3-difluoro-5-iodophenyl)methanol (9.15 g, 33.9 mmol) in THF (68 mL) at 0° C. The mixture was stirred at 0° C. for 5 minutes and o-bromo (bromomethyl)benzene (8.47 g, 33.9 mmol) was added. The reaction mixture was allowed to warm to room temperature and stirred for 1 hour. The reaction mixture was diluted with EtOAc and washed with brine (2×). The organic layer was dried over Na₂SO₄ and concentrated in vacuo. The crude product was purified by column chromatography (silica gel, 0-10% EtOAc in heptane) to afford 3-{[(o-bromophenyl)methoxy]methyl}-1,2-difluoro-5-iodobenzene (12.1 g, 27.7 mmol) as a clear oil. ¹H NMR (400 MHz, CHLOROFORM-D) δ 7.63-7.40 (m, 4H), 7.37-7.26 (m, 1H), 7.21-7.05 (m, 1H), 4.68-4.61 (m, 4H).

Step C: A mixture of 3-{[(o-bromophenyl)methoxy]methyl}-1,2-difluoro-5-iodobenzene (12.1 g, 27.7 mmol), tert-butyl carbazate (4.75 g, 35.9 mmol), cesium carbonate (12.6 g, 38.7 mmol), copper iodide (263 mg, 1.38 mmol), and 1,10-phenanthroline (498 mg, 2.77 mmol) in DMF (28 mL) was sparged with nitrogen for 15 minutes. The reaction mixture was stirred at 80° C. for 4.5 hours. The reaction mixture was allowed to cool to room temperature, diluted with EtOAc, and filtered through a pad of celite. The filtrate was washed with brine (2×). The organic layer was dried over Na₂SO₄ and concentrated in vacuo. The crude residue was purified by column chromatography (silica gel, 0-40% EtOAc in heptane) to afford tert-butyl 2-(5-{[(o-bromophenyl)methoxy]methyl}-3,4-difluorophenyl)carbazate (6.3 g, 14.2 mmol) as a yellow oil. m/z (ESI-pos) M+1=387.1/389.1 (loss of tBu).

Step D: To a stirred solution of tert-butyl 2-(5-{[(o-bromophenyl)methoxy]methyl}-3,4-difluorophenyl)carbazate (6.3 g, 14.2 mmol) in EtOAc (40 mL) and heptane (40 mL) was added 4M HCl in dioxane (27.7 mL, 111 mmol). The reaction mixture was stirred at 45° C. overnight. The reaction mixture was allowed to cool to room temperature, the resulting solids were filtered, washed with heptane, and dried in vacuo to afford 5-{[(o-bromophenyl)methoxy]methyl}-3,4-difluorophenylaminoammonium chloride (4.96, 13.1 mmol) as a pink solid. m/z (ESI-pos) M+1=343.1/345.1

Step E: To a stirred solution of 5-{[(o-bromophenyl)methoxy]methyl}-3,4-difluorophenylaminoammonium chloride (4.96 g, 13.1 mmol), tert-butyl(2S)-3-cyano-2-methyl-4-oxo-1-piperidinecarboxylate (3.11 g, 13.1 mmol), and sodium acetate (1.07 g, 13.1 mmol) in ethanol (38 mL) was added acetic acid (748 μL, 13.1 mmol). The reaction mixture was stirred at 60° C. overnight. The reaction mixture was allowed to cool to room temperature, quenched with saturated aqueous Na₂CO₃ and extracted with EtOAc. The organic layer was washed with brine, dried over Na₂SO₄, and concentrated in vacuo. The crude compound was purified by column chromatography (silica gel, 10-50% EtOAc in heptane) to afford tert-butyl (S)-3-amino-2-(5-{[(o-bromophenyl)methoxy]methyl}-3,4-difluorophenyl)-4-methyl-1,2,5-triaza-2,4,6,7-tetrahydro-5H-indene-5-carboxylate (5.79 g, 10.3 mmol). m/z (ESI-pos) M+1=563.3/565.3

Step F: To a stirred solution of tert-butyl (S)-3-amino-2-(5-{[(o-bromophenyl)methoxy]methyl}-3,4-difluorophenyl)-4-methyl-1,2,5-triaza-2,4,6,7-tetrahydro-5H-indene-5-carboxylate (5.79 g, 10.3 mmol) in tetrahydrofuran (51 mL) was added N-ethyldiisopropylamine (5.37 mL, 30.8 mmol) and benzenyl chloroformate (2.58 mL, 20.6 mmol). The reaction mixture was stirred at room temperature for 1 hour. The reaction mixture was concentrated in vacuo to give tert-butyl (S)-2-(5-{[(o-bromophenyl)methoxy]methyl}-3,4-difluorophenyl)-4-methyl-3-[phenyl(oxycarbonylamino)]-1,2,5-triaza-2,4,6,7-tetrahydro-5H-indene-5-carboxylate as a yellow oil, which was used directly in the next step without purification.

Step G: To a stirred solution of tert-butyl (S)-2-(5-{[(o-bromophenyl)methoxy]methyl}-3,4-difluorophenyl)-4-methyl-3-[phenyl(oxycarbonylamino)]-1,2,5-triaza-2,4,6,7-tetrahydro-5H-indene-5-carboxylate in pyridine (51 mL) was added 2,2-dimethoxyethylamine (4.48 mL, 41.1 mmol) at room temperature. The reaction mixture was stirred at room temperature for 30 minutes and concentrated in vacuo. The crude product was purified by column chromatography (silica gel, 10-60% EtOAc in heptane) to afford tert-butyl (S)-2-(5-{[(o-bromophenyl)methoxy]methyl}-3,4-difluorophenyl)-3-[3-(2,2-dimethoxyethyl)ureido]-4-methyl-1,2,5-triaza-2,4,6,7-tetrahydro-5H-indene-5-carboxylate (6.25 g, 9 mmol).

Step H: To a solution of tert-butyl (S)-2-(5-{[(o-bromophenyl)methoxy]methyl}-3,4-difluorophenyl)-3-[3-(2,2-dimethoxyethyl)ureido]-4-methyl-1,2,5-triaza-2,4,6,7-tetrahydro-5H-indene-5-carboxylate (6.25 g, 9 mmol) in tetrahydrofuran (45 mL) was added methanesulfonic acid (1.75 mL, 27 mmol). The mixture was stirred at 55° C. for 1 hour then allowed to cool to ambient temperature. Saturated aqueous NaHCO₃ (45 mL) and tert-butyl-tert-butyl(oxycarbonyloxy) formylate (2.95 g, 13.5 mmol) were added, and the mixture was stirred at room temperature for 30 minutes. The mixture was extracted with DCM, dried over anhydrous Na₂SO₄, and concentrated in vacuo. The crude residue was purified by column chromatography (silica gel, 10-80% EtOAc in heptane) to give tert-butyl (S)-2-(5-{[(0-bromophenyl)methoxy]methyl}-3,4-difluorophenyl)-4-methyl-3-(2-oxo-2,3-dihydro-1H-imidazol-1-yl)-1,2,5-triaza-2,4,6,7-tetrahydro-5H-indene-5-carboxylate (5.41 g, 8.58 mmol) as a white solid. m/z (ESI-pos) M+1=630.3, 632.3

Step I: A solution of tert-butyl (S)-2-(5-{[(o-bromophenyl)methoxy]methyl}-3,4-difluorophenyl)-4-methyl-3-(2-oxo-2,3-dihydro-1H-imidazol-1-yl)-1,2,5-triaza-2,4,6,7-tetrahydro-5H-indene-5-carboxylate (1.5 g, 2.38 mmol), copper iodide (340 mg, 1.78 mmol), bathophenanthroline (791 mg, 2.38 mmol), and dipotassium carbonate (822 mg, 5.95 mmol) in dimethyl sulfoxide (79.3 mL) was sparged with N₂ for 15 minutes. The reaction was stirred at 130° C. for 21 hours. The reaction mixture was allowed to cool to room temperature then filtered through a pad of Celite with EtOAc. The filtrate was washed with saturated aqueous NH₄Cl and washed with brine. The organic layer was dried over sodium sulfate and concentrated in vacuo. The crude residue was purified by column chromatography (silica gel, 5-40% EtOAc in heptane) to give tert-butyl (8S,10M)-25,26-difluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (270 mg, 491 μmol). m/z (ESI-pos) M+1=550.4

Step J: 4M HCl in dioxane (0.5 mL, 910 μmol) was added to a stirred solution of tert-butyl (8S,10M)-25,26-difluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (25 mg, 45.5 μmol) in dichloromethane (0.25 mL) at room temperature. The solution was stirred at room temperature for 40 minutes and concentrated in vacuo to provide (8S,10M)-25,26-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (22.1 mg, 45.5 μmol). m/z (ESI-pos) M+1=450.3

Step K: To a solution of (8S,10M)-25,26-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (22.1 mg, 45.5 μmol) and 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid (25.1 mg, 59.1 μmol) in dimethylformamide (0.6 mL) was added N-ethyldiisopropylamine (31.7 μL, 182 μmol) and HATU (24.2 mg, 63.7 μmol) at room temperature. The mixture stirred at room temperature for 1 hour. The mixture was diluted with EtOAc, washed with saturated aqueous NH₄Cl, washed with brine, dried over anhydrous Na₂SO₄, and concentrated in vacuo. The crude residue was purified by reverse-phase chromatography (40-100% MeCN in water with 0.1% FA) to provide (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-25,26-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (4.39 mg, 4.98 μmol) as a white solid. m/z (ESI-pos) M+1=856.6; ¹H NMR (400 MHz, CHLOROFORM-D) δ 11.33-11.21 (m, 1H), 7.60-7.32 (m, 6H), 7.19-7.09 (m, 2H), 7.03-6.89 (m, 1H), 6.77-6.63 (m, 1H), 6.55-5.90 (m, 2H), 5.85-5.24 (m, 1H), 4.96-4.66 (m, 2H), 4.56-4.27 (m, 2H), 4.19-4.10 (m, 1H), 4.10-4.00 (m, 1H), 3.80 (td, J=11.4, 2.5 Hz, 1H), 3.63 (td, J=13.3, 4.1 Hz, 1H), 3.53 (d, J=11.5 Hz, 1H), 3.46-3.30 (m, 1H), 3.21-2.80 (m, 4H), 2.72 (dd, J=11.6, 10.1 Hz, 1H), 1.93-1.82 (m, 1H), 1.81-1.08 (m, 8H), 0.97 (dtd, J=13.2, 8.4, 4.9 Hz, 1H), 0.69-0.52 (m, 2H), 0.47 (dq, J=9.3, 4.7 Hz, 1H), 0.30 (dq, J=9.5, 4.7 Hz, 1H).

### Example 207—Synthesis of Compound 207

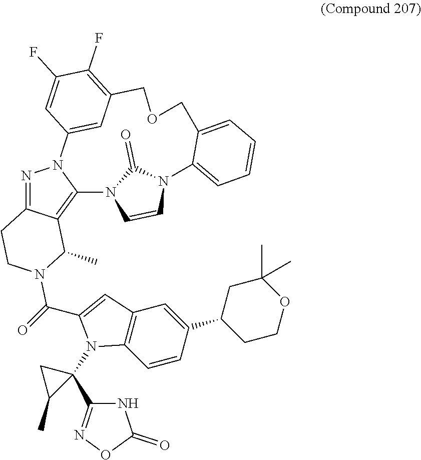

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25,26-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 207 was synthesized following the procedure of Example 206, with 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid in step K. m/z (ESI-pos) M+1=843.6; ¹H NMR (400 MHz, CHLOROFORM-D) δ 11.37-11.18 (m, 1H), 7.63-7.27 (m, 8H), 7.03-6.90 (m, 1H), 6.81-6.68 (m, 1H), 6.55-5.90 (m, 2H), 5.87-5.20 (m, 1H), 4.98-4.44 (m, 3H), 4.43-4.27 (m, 1H), 4.21-4.07 (m, 1H), 3.94-3.74 (m, 2H), 3.70-3.35 (m, 1H), 3.25-2.90 (m, 3H), 1.96-1.37 (m, 10H), 1.32 (d, J=23.8 Hz, 6H), 1.24-1.09 (m, 3H).

### Example 208—Synthesis of Compound 208

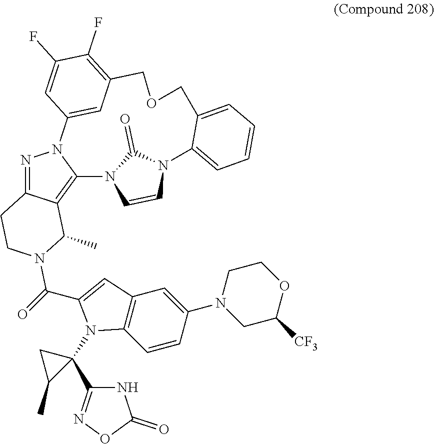

### (8S,10M)-25,26-difluoro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 208 was synthesized following the procedure of Example 206, with 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid in step K. m/z (ESI-pos) M+1=884.6; ¹H NMR (400 MHz, CHLOROFORM-D) δ 11.35-11.20 (m, 1H), 7.62-7.32 (m, 6H), 7.22-7.10 (m, 2H), 7.04-6.89 (m, 1H), 6.79-6.64 (m, 1H), 6.55-5.90 (m, 2H), 5.86-5.22 (m, 1H), 4.98-4.64 (m, 2H), 4.55-4.26 (m, 2H), 4.23-4.08 (m, 3H), 3.91 (td, J=11.4, 2.6 Hz, 1H), 3.72-3.49 (m, 2H), 3.37 (d, J=12.0 Hz, 1H), 3.22-2.80 (m, 4H), 1.95-1.83 (m, 1H), 1.82-1.33 (m, 5H), 1.32-1.10 (m, 3H).

### Example 209—Synthesis of Compound 209

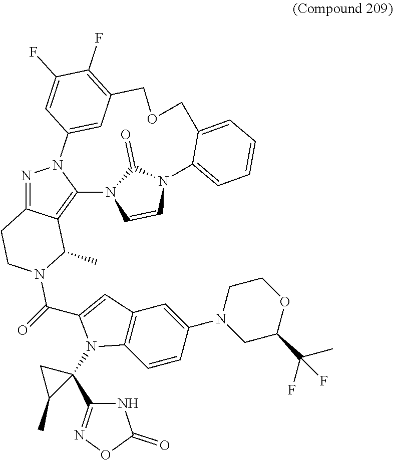

### (8S,10M)-7-({5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-25,26-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 209 was synthesized following the procedure of Example 206, with 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid being replaced with 5-[(R)-2-(1,1-difluoroethyl)-4-morpholinyl]-1-[(1S,2S)-2-methyl-1-(5-oxo-4,5-dihydro-1,2,4-oxadiazol-3-yl)cyclopropyl]-2-indolecarboxylic acid in step K. m/z (ESI-pos) M+1=880.6; ¹H NMR (400 MHz, CHLOROFORM-D) δ 11.38-11.15 (m, 1H), 7.61-7.32 (m, 6H), 7.21-7.10 (m, 2H), 7.02-6.89 (m, 1H), 6.77-6.64 (m, 1H), 6.55-5.90 (m, 2H), 5.86-5.22 (m, 1H), 4.96-4.66 (m, 2H), 4.56-4.27 (m, 2H), 4.19-4.05 (m, 2H), 3.95-3.79 (m, 2H), 3.70-3.54 (m, 2H), 3.45-3.32 (m, 1H), 3.21-2.96 (m, 2H), 2.89 (td, J=11.8, 3.3 Hz, 1H), 2.79 (t, J=11.0 Hz, 1H), 1.94-1.82 (m, 1H), 1.80-1.25 (m, 8H), 1.25-1.10 (m, 3H).

### Example 210—Synthesis of Compound 210

### Synthetic Scheme

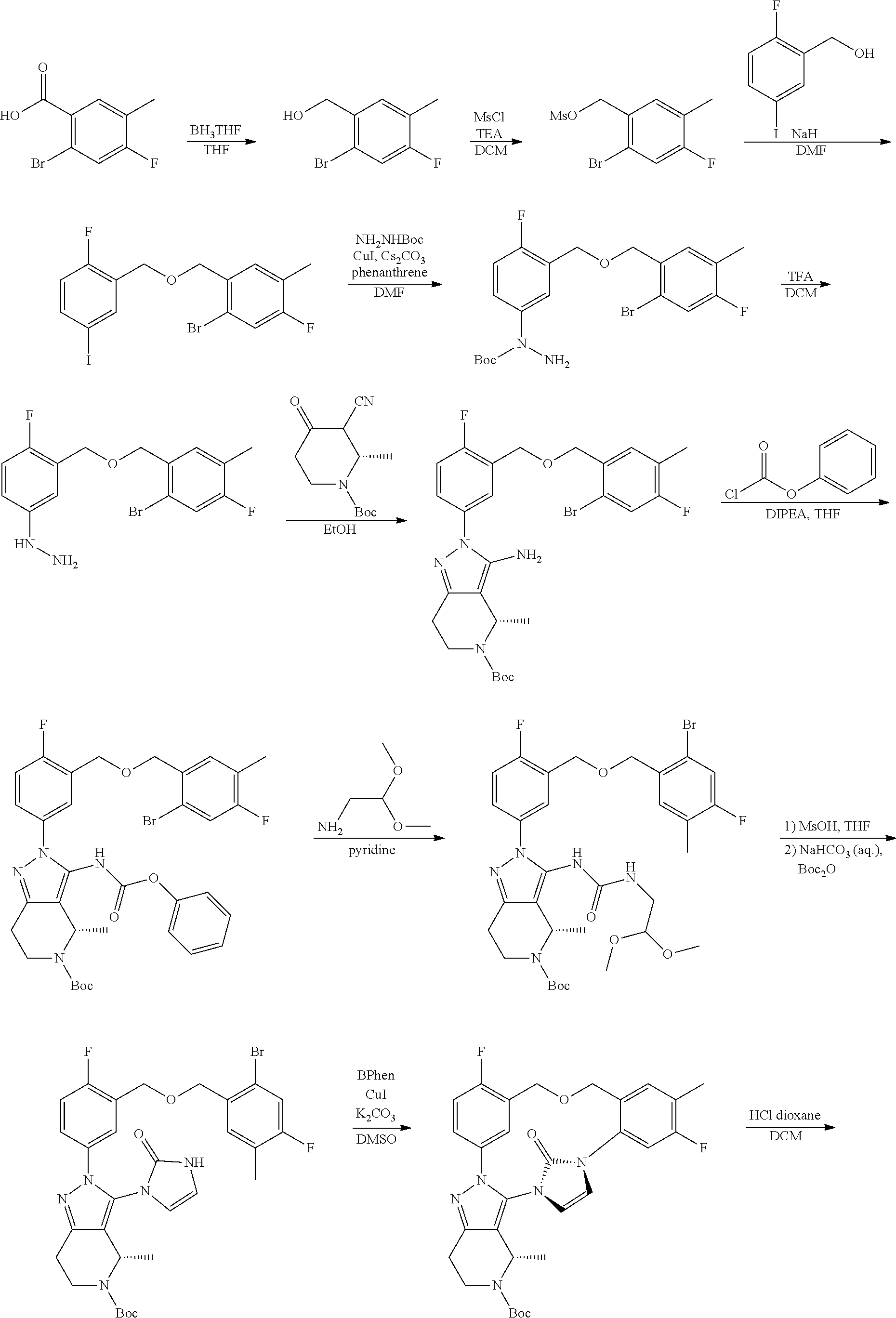

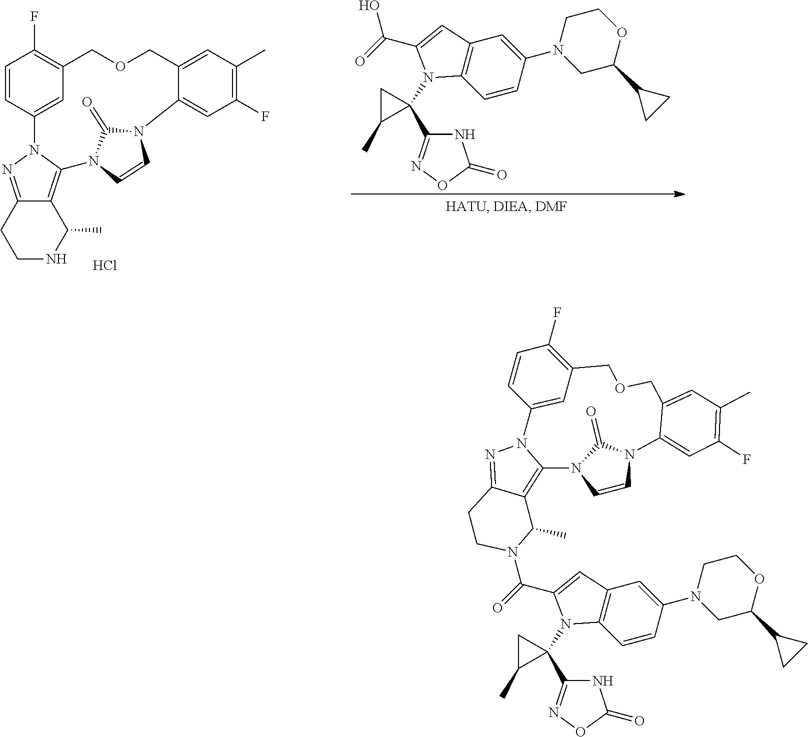

### Procedure for Synthesis of Compound 210

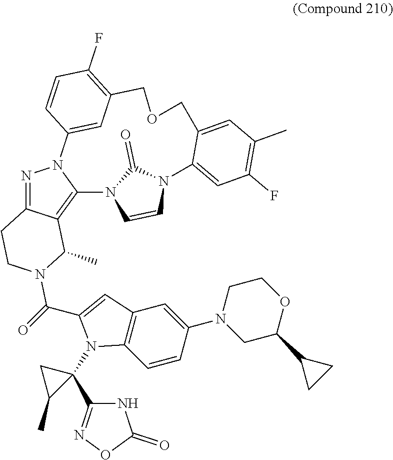

### (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: 1M Borane in THF (129 mL, 129 mmol) was added dropwise to a stirred solution of 4-bromo-6-fluoro-3-toluic acid (15 g, 64.4 mmol) in tetrahydrofuran (161 mL) at 0° C. The reaction was stirred at 0° C. for 30 minutes and at room temperature for 2 hours. The reaction mixture was cooled 0° C., quenched with saturated aqueous NaHCO₃, diluted with EtOAc, and washed with brine (2×). The organic layer was dried over Na₂SO₄ and concentrated in vacuo to afford (2-bromo-4-fluoro-5-tolyl)methanol (14 g, 63.9 mmol).

Step B: To a solution of (2-bromo-4-fluoro-5-tolyl)methanol (5 g, 22.8 mmol) and triethylamine (4.77 mL, 34.2 mmol) in dichloromethane (114 mL) at 0° C. was added (chlorosulfonyl)methane (2 mL, 25.8 mmol). The mixture was stirred at 0° C. for 2 hours. The reaction was diluted with water. The organic layer was separated, washed with brine, dried over Na₂SO₄, filtered, and concentrated in vacuo to afford 4-bromo-2-fluoro-5-[(mesyloxy)methyl]toluene which was used directly in the next step.

Step C: Sodium hydride 60% w/w (1.37 g, 34.2 mmol) was added to a solution of (2-fluoro-5-iodophenyl)methanol (5.75 g, 22.8 mmol) in DMF (38 mL) at 0° C. The solution was stirred at 0° C. for 15 minutes. A solution of 4-bromo-2-fluoro-5-[(mesyloxy)methyl]toluene in DMF (38 mL) was added and the reaction was stirred at 0° C. for 1 hour. The reaction mixture was quenched with sat. aq. NH₄Cl, diluted with ethyl acetate, washed with brine (2×). The organic layer was dried over Na₂SO₄, filtered, and concentrated in vacuo. The crude product was purified by column chromatography (silica gel, 0-15% EA in heptane) to give 4-bromo-2-fluoro-5-{[(2-fluoro-5-iodophenyl)methoxy]methyl}toluene (8.45 g, 18.7 mmol).

Step D: A mixture of 4-bromo-2-fluoro-5-{[(2-fluoro-5-iodophenyl)methoxy]methyl}toluene (8.45 g, 18.7 mmol), tert-butyl carbazate (2.96 g, 22.4 mmol), cesium carbonate (8.51 g, 26.1 mmol), copper iodide (355 mg, 1.87 mmol), and 1,10-phenanthroline (1.34 g, 7.46 mmol) in DMF (20 mL) was sparged with N₂ for 10 minutes. The reaction mixture was stirred at 80° C. overnight. The reaction mixture was allowed to cool to room temperature, diluted with EtOAc, and filtered through a pad of Celite. The filtrate was washed with brine (2×). The organic layer was dried over Na₂SO₄ and concentrated in vacuo. The crude residue was purified by column chromatography (silica gel, 0-50% EtOAc in heptane) to afford tert-butyl 3-(3-{[(2-bromo-4-fluoro-5-tolyl)methoxy]methyl}-4-fluorophenyl)carbazate (5.95 g, 13 mmol). m/z (ESI-pos) M+1=401.1, 403.1 (loss of tBu).

Step E: Trifluoroacetic acid (14.9 mL, 195 mmol) was added to a stirred solution of tert-butyl 3-(3-{[(2-bromo-4-fluoro-5-tolyl)methoxy]methyl}-4-fluorophenyl)carbazate (5.95 g, 13 mmol) in dichloromethane (30 mL) at room temperature. The reaction was stirred at room temperature for 3 hours. The reaction was quenched with sat. aq. Na₂CO₃. The organics were separated, washed with brine, dried over Na₂SO₄, filtered, and concentrated in vacuo to provide (3-{[(2-bromo-4-fluoro-5-tolyl)methoxy]methyl}-4-fluorophenyl)hydrazine (4.65 g, 13 mmol). m/z (ESI-pos) M+1=357.1, 359.1.

Step F: Tert-butyl(2S)-3-cyano-2-methyl-4-oxo-1-piperidinecarboxylate (3.1 g, 13 mmol) was added to a stirred solution of (3-{[(2-bromo-4-fluoro-5-tolyl)methoxy]methyl}-4-fluorophenyl)hydrazine (4.65 g, 13 mmol) in ethanol (26 mL) at room temperature. The mixture was stirred at 80° C. for 18 hours. The reaction mixture was allowed to cool to room temperature and concentrated in vacuo. The crude compound was purified by column chromatography (silica, 10-50% EtOAc/heptane) to afford tert-butyl (S)-3-amino-2-(3-{[(2-bromo-4-fluoro-5-tolyl)methoxy]methyl}-4-fluorophenyl)-4-methyl-1,2,5-triaza-2,4,6,7-tetrahydro-5H-indene-5-carboxylate (4.66 g, 8.07 mmol). m/z (ESI-pos) M+1=577.3, 579.3.

Step G: To a stirred solution of tert-butyl (S)-3-amino-2-(3-{[(2-bromo-4-fluoro-5-tolyl)methoxy]methyl}-4-fluorophenyl)-4-methyl-1,2,5-triaza-2,4,6,7-tetrahydro-5H-indene-5-carboxylate (4.66 g, 8.07 mmol) in tetrahydrofuran (40 mL) was added N-ethyldiisopropylamine (4.22 mL, 24.2 mmol) followed by benzenyl chloroformate (2.02 mL, 16.1 mmol). The reaction mixture was stirred at room temperature for 30 minutes. The reaction mixture was concentrated in vacuo to give crude tert-butyl (S)-2-(3-{[(2-bromo-4-fluoro-5-tolyl)methoxy]methyl}-4-fluorophenyl)-4-methyl-3-[phenyl(oxycarbonylamino)]-1,2,5-triaza-2,4,6,7-tetrahydro-5H-indene-5-carboxylate which was used directly in the next step. m/z (ESI-pos) M+1=697.3, 699.4

Step H: 2,2-Dimethoxyethylamine (3.52 mL, 32.3 mmol) was added to a stirred solution of crude tert-butyl (S)-2-(3-{[(2-bromo-4-fluoro-5-tolyl)methoxy]methyl}-4-fluorophenyl)-4-methyl-3-[phenyl(oxycarbonylamino)]-1,2,5-triaza-2,4,6,7-tetrahydro-5H-indene-5-carboxylate in pyridine (40 mL). The mixture was stirred at room temperature for 30 minutes and concentrated in vacuo. The residue was diluted with EtOAc and washed with sat. aq. NaHCO₃. The organic layer was dried over Na₂SO₄, filtered, and concentrated in vacuo. The crude product was purified by column chromatography (silica, 10-60% EtOAc in heptane) to give tert-butyl (S)-2-(3-{[(2-bromo-4-fluoro-5-tolyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (4.86 g, 6.86 mmol). m/z (ESI-pos) M+1=708.4, 710.4.

Step I: To a solution of tert-butyl (S)-2-(3-{[(2-bromo-4-fluoro-5-tolyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (4.86 g, 6.86 mmol) in tetrahydrofuran (70 mL) was added methanesulfonic acid (2.23 mL, 34.3 mmol). The mixture was stirred at 55° C. for 1 hour then allowed to cool to ambient temperature. Saturated aqueous NaHCO₃ (70 mL) and tert-butyl-tert-butyl(oxycarbonyloxy) formylate (2.99 g, 13.7 mmol) were added, and the mixture was stirred at room temperature for 1 hour. The mixture was extracted with DCM, dried over anhydrous Na₂SO₄, and concentrated in vacuo. The crude residue was purified by column chromatography (silica gel, 10-80% EtOAc in heptane) to give tert-butyl (S)-2-(3-{[(2-bromo-4-fluoro-5-tolyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (3.92 g, 6.08 mmol). m/z (ESI-pos) M+1=644.3, 646.3.

Step J: A solution of tert-butyl (S)-2-(3-{[(2-bromo-4-fluoro-5-tolyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (1 g, 1.55 mmol), copper iodide (222 mg, 1.16 mmol), bathophenanthroline (516 mg, 1.55 mmol), and dipotassium carbonate (536 mg, 3.88 mmol) in dimethyl sulfoxide (52 mL) was sparged with N₂ for 30 minutes. The reaction was stirred at 130° C. for 40 hours. The reaction mixture was allowed to cool to room temperature then filtered through a pad of Celite with EtOAc. The filtrate was washed with saturated aqueous NH₄Cl and washed with brine (2×). The organic layer was dried over sodium sulfate and concentrated in vacuo. The crude residue was purified by column chromatography (silica gel, 5-40% EtOAc in heptane) to give tert-butyl (8S,10M)-17,25-difluoro-8,18-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (169 mg, 299 μmol). m/z (ESI-pos) M+1=564.4.

Step K: 4M HCl in dioxane (0.75 mL) was added to a stirred solution of tert-butyl (8S,10M)-17,25-difluoro-8,18-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (43.3 mg, 76.8 μmol) in dichloromethane (0.75 mL) at room temperature. The reaction was stirred at room temperature for 2 hours and concentrated in vacuo to provide (8S,10M)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (23.1 mg, 46.2 μmol). m/z (ESI-pos) M+1=464.3.

Step L: To a solution of (8S,10M)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (23.1 mg, 46.2 μmol) and 5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolecarboxylic acid (21.6 mg, 50.8 μmol) in dimethylformamide (0.8 mL) was added N-ethyldiisopropylamine (32.2 μL, 185 μmol) and HATU (24.6 mg, 64.7 μmol) at room temperature. The mixture stirred at room temperature overnight. The mixture was diluted with EtOAc, washed with saturated aqueous NH₄Cl, washed with brine (2×), dried over anhydrous Na₂SO₄, and concentrated in vacuo. The crude residue was purified by reverse-phase chromatography (40-100% MeCN in water with 0.1% FA) to provide (8S,10M)-7-({5-[(S)-2-cyclopropyl-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (12.2 mg, 13.8 μmol) as a white solid. m/z (ESI-pos) M+1=870.6; ¹H NMR (400 MHz, CHLOROFORM-D) δ 11.38-11.23 (m, 1H), 7.70-7.50 (m, 2H), 7.44-7.35 (m, 1H), 7.35-7.27 (m, 1H), 7.25-6.98 (m, 4H), 6.76-6.63 (m, 1H), 6.42-5.85 (m, 2H), 5.84-5.23 (m, 1H), 4.96-4.63 (m, 2H), 4.49 (dd, J=13.8, 5.1 Hz, 1H), 4.27-4.01 (m, 3H), 3.79 (td, J=11.4, 2.6 Hz, 1H), 3.64 (td, J=13.1, 4.3 Hz, 1H), 3.56-3.29 (m, 2H), 3.24-2.79 (m, 4H), 2.77-2.66 (m, 1H), 2.40-2.28 (m, 3H), 1.99-1.07 (m, 9H), 1.03-0.88 (m, 1H), 0.69-0.52 (m, 2H), 0.47 (dq, J=9.2, 4.8 Hz, 1H), 0.30 (dq, J=9.6, 4.8 Hz, 1H).

### Example 211—Synthesis of Compound 211

### Synthetic Scheme

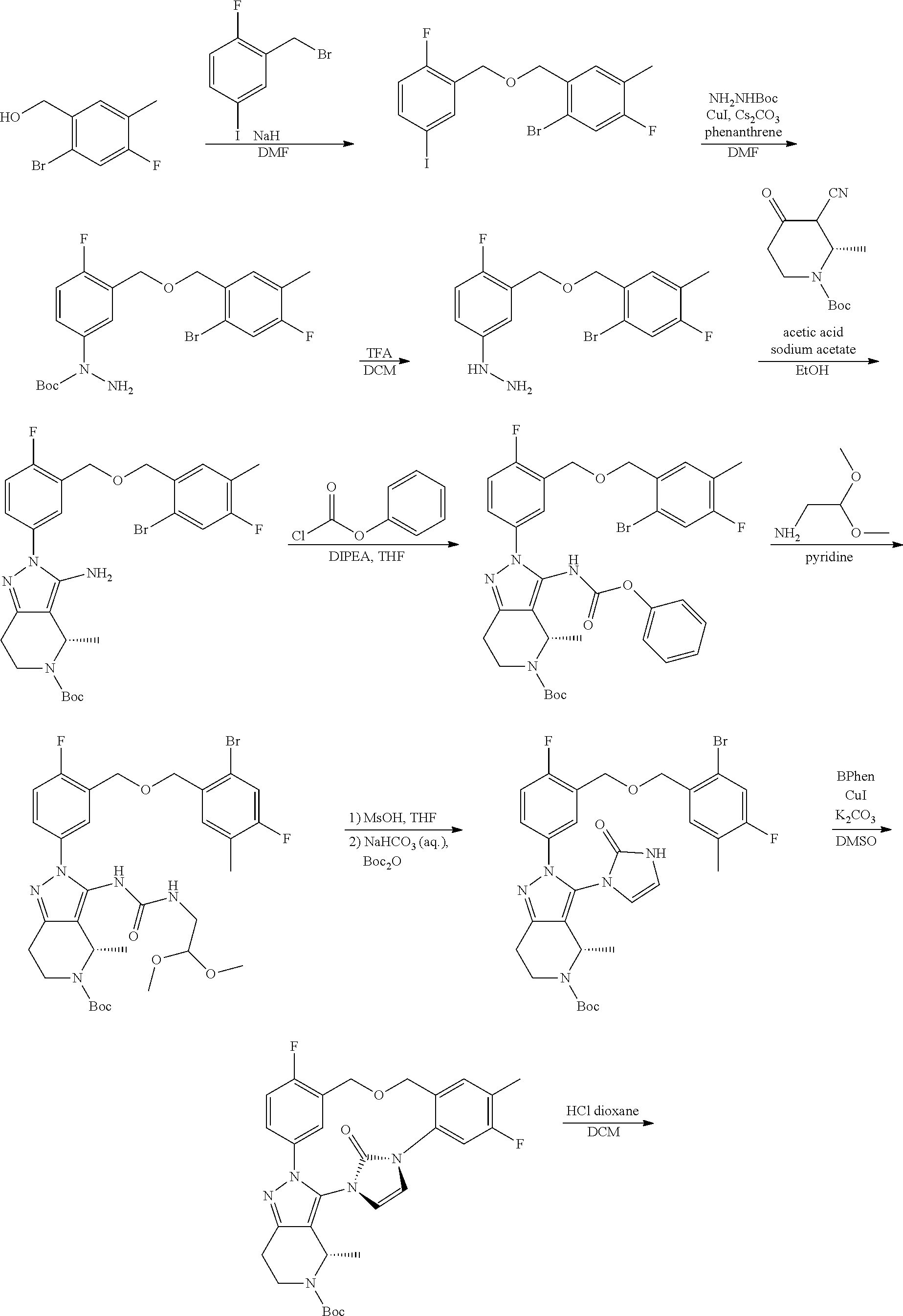

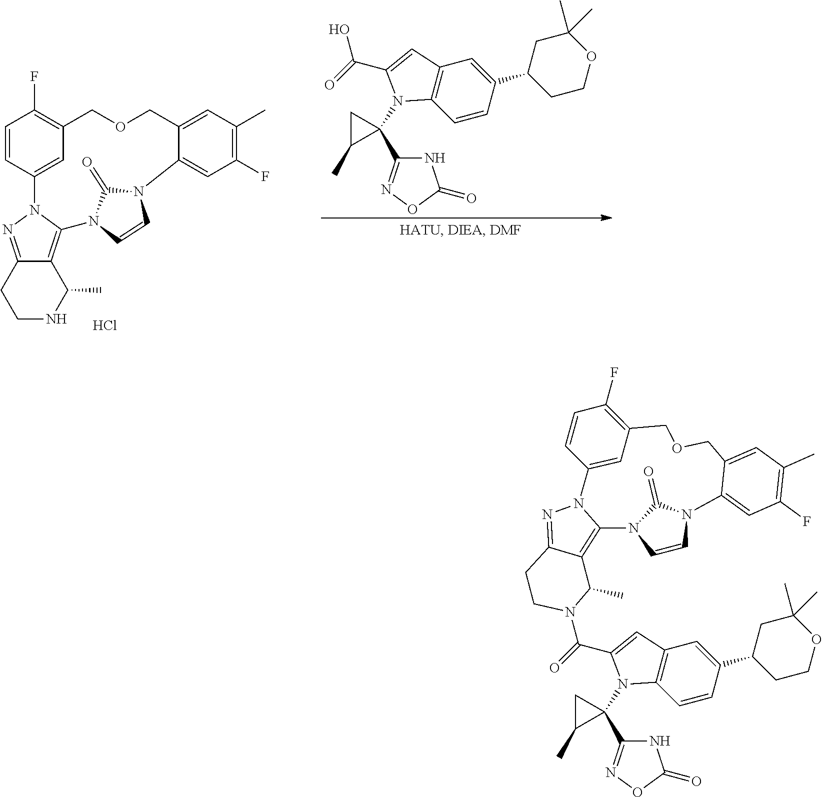

### Procedure for Synthesis of Compound 211

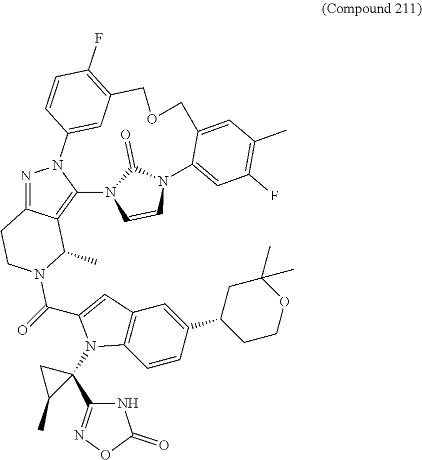

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a mixture of (2-bromo-4-fluoro-5-tolyl)methanol (560 mg, 2.56 mmol) in dimethylformamide (8.5 mL) was added sodium hydride 60% w/w (153 mg, 3.83 mmol) at 0° C. The reaction was stirred at 0° C. for 20 minutes and 2-(bromomethyl)-1- fluoro-4-iodobenzene (926 mg, 2.94 mmol) was added portion-wise. The reaction mixture was allowed to warm to room temperature and stirred for 1 hour. The mixture was slowly quenched with saturated aqueous NH₄Cl and extracted with EtOAc. The organics were washed with brine (2×), dried over Na₂SO₄, filtered, and concentrated in vacuo. The crude product was purified by column chromatography (silica gel, 0-15% EA in heptane) to give 4-bromo-2-fluoro-5-{[(2-fluoro-5-iodophenyl)methoxy]methyl}toluene (1.13 g, 2.48 mmol). ¹H NMR (400 MHz, CHLOROFORM-D) δ 7.81 (dt, J=6.8, 1.6 Hz, 1H), 7.61-7.52 (m, 1H), 7.31 (d, J=8.4 Hz, 1H), 7.22 (d, J=8.9 Hz, 1H), 6.82 (dd, J=9.6, 8.6 Hz, 1H), 4.67-4.56 (m, 4H), 2.25 (d, J=2.5 Hz, 3H).

Step B: A mixture of 4-bromo-2-fluoro-5-{[(2-fluoro-5-iodophenyl)methoxy]methyl}toluene (1.13 g, 2.48 mmol), tert-butyl carbazate (394 mg, 2.98 mmol), cesium carbonate (1.13 g, 3.48 mmol), copper iodide (47.3 mg, 248 μmol), and 1,10-phenanthroline (179 mg, 993 μmol) in DMF (3.15 mL) was sparged with N₂ for 5 minutes. The reaction mixture was stirred at 80° C. for 8 hours. The reaction mixture was allowed to cool to room temperature, diluted with EtOAc, and filtered through a pad of celite. The filtrate was washed with brine (2×). The organic layer was dried over Na₂SO₄ and concentrated in vacuo. The crude residue was purified by column chromatography (silica gel, 10-50% EtOAc in heptane) to afford tert-butyl 3-(3-{[(2-bromo-4-fluoro-5-tolyl)methoxy]methyl}-4-fluorophenyl)carbazate (715 mg, 1.56 mmol). m/z (ESI-pos) M+1=401.1, 403.1 (loss of tBu).

Step C: Trifluoroacetic acid (1.2 mL, 15.6 mmol) was added to a stirred solution of tert-butyl 3-(3-{[(2-bromo-4-fluoro-5-tolyl)methoxy]methyl}-4-fluorophenyl)carbazate (715 mg, 1.56 mmol) in dichloromethane (5.2 mL) at room temperature. The reaction was stirred at room temperature for 2.5 hours. The reaction was quenched with sat. aq. Na₂CO₃, extracted with DCM, washed with brine, dried over Na₂SO₄, filtered, and concentrated in vacuo to provide (3-{[(2-bromo-4-fluoro-5-tolyl)methoxy]methyl}-4-fluorophenyl)hydrazine (559 mg, 1.56 mmol). m/z (ESI-pos) M+1=357.1, 359.1.

Step D: Tert-butyl(2S)-3-cyano-2-methyl-4-oxo-1-piperidinecarboxylate (447 mg, 1.88 mmol) and N-ethyldiisopropylamine (409 μL, 2.35 mmol) were added to a stirred solution of (3-{[(2-bromo-4-fluoro-5-tolyl)methoxy]methyl}-4-fluorophenyl)hydrazine (559 mg, 1.56 mmol) in ethanol (5.2 mL) at room temperature. The mixture was stirred at 80° C. for 8 hours. The reaction mixture was allowed to cool to room temperature, concentrated in vacuo, diluted with water, and extracted with DCM (2×). The organic layers were dried over anhydrous Na₂SO₄ and concentrated in vacuo. The crude compound was purified by column chromatography (silica, 10-50% EtOAc/heptane) to afford tert-butyl (S)-3-amino-2-(3-{[(2-bromo-4-fluoro-5-tolyl)methoxy]methyl}-4-fluorophenyl)-4-methyl-1,2,5-triaza-2,4,6,7-tetrahydro-5H-indene-5-carboxylate (272 mg, 471 μmol). m/z (ESI-pos) M+1=577.3, 579.3.

Step E: To a stirred solution of tert-butyl (S)-3-amino-2-(3-{[(2-bromo-4-fluoro-5-tolyl)methoxy]methyl}-4-fluorophenyl)-4-methyl-1,2,5-triaza-2,4,6,7-tetrahydro-5H-indene-5-carboxylate (272 mg, 471 μmol) in tetrahydrofuran (2.4 mL) was added N-ethyldiisopropylamine (246 μL, 1.41 mmol) followed by benzenyl chloroformate (118 μL, 942 μmol). The reaction mixture was stirred at room temperature for 30 minutes. The reaction mixture was concentrated in vacuo to give crude tert-butyl (S)-2-(3-{[(2-bromo-4-fluoro-5-tolyl)methoxy]methyl}-4-fluorophenyl)-4-methyl-3-[phenyl(oxycarbonylamino)]-1,2,5-triaza-2,4,6,7-tetrahydro-5H-indene-5-carboxylate which was used directly in the next step. m/z (ESI-pos) M+1=697.4, 699.4.

Step F: 2,2-Dimethoxyethylamine (205 μL, 1.88 mmol) was added to a stirred solution of crude tert-butyl (S)-2-(3-{[(2-bromo-4-fluoro-5-tolyl)methoxy]methyl}-4-fluorophenyl)-4-methyl-3-[phenyl(oxycarbonylamino)]-1,2,5-triaza-2,4,6,7-tetrahydro-5H-indene-5-carboxylate in pyridine (2.35 mL). The mixture was stirred at room temperature for 30 minutes and concentrated in vacuo. The crude product was purified by column chromatography (silica, 10-80% EtOAc in heptane) to give tert-butyl (S)-2-(3-{[(2-bromo-4-fluoro-5-tolyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (286 mg, 404 μmol). m/z (ESI-pos) M+1=708.4, 710.4.

Step G: To a solution of tert-butyl (S)-2-(3-{[(2-bromo-4-fluoro-5-tolyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (286 mg, 404 μmol) in tetrahydrofuran (4 mL) was added methanesulfonic acid (131 μL, 2.02 mmol). The mixture was stirred at 55° C. for 1 hour then allowed to cool to ambient temperature. Saturated aqueous NaHCO₃ (4 mL) and tert-butyl-tert-butyl(oxycarbonyloxy) formylate (176 mg, 808 μmol) were added, and the mixture was stirred at room temperature for 30 minutes. The mixture was extracted with DCM, dried over anhydrous Na₂SO₄, and concentrated in vacuo. The crude residue was purified by column chromatography (silica gel, 10-80% EtOAc in heptane) to give tert-butyl (S)-2-(3-{[(2-bromo-4-fluoro-5-tolyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (260 mg, 403 μmol). m/z (ESI-pos) M+1=644.3, 646.3.

Step H: A solution of tert-butyl (S)-2-(3-{[(2-bromo-4-fluoro-5-tolyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (260 mg, 403 μmol), copper iodide (57.6 mg, 302 μmol), bathophenanthroline (134 mg, 403 μmol), and dipotassium carbonate (139 mg, 1.01 mmol) in dimethyl sulfoxide (13 mL) was sparged with N₂ for 10 minutes. The reaction was stirred at 130° C. for 40 hours. The reaction mixture was allowed to cool to room temperature then filtered through a pad of Celite with EtOAc. The filtrate was washed with saturated aqueous NH₄Cl and washed with brine. The organic layer was dried over sodium sulfate and concentrated in vacuo. The crude residue was purified by column chromatography (silica gel, 5-40% EtOAc in heptane) to give tert-butyl (8S,10M)-17,25-difluoro-8,18-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (43.3 mg, 76.8 μmol). m/z (ESI-pos) M+1=564.4.

Step I: 4M HCl in dioxane (1 mL) was added to a stirred solution of tert-butyl (8S,10M)-17,25-difluoro-8,18-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-temperature. The solution was stirred at room temperature for 1 hour and concentrated in vacuo to provide (8S,10M)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (38.4 mg, 76.8 μmol). m/z (ESI-pos) M+1=464.3.

Step J: To a solution of (8S,10M)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (38.4 mg, 76.8 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (41.1 mg, 99.9 μmol) in dimethylformamide (1.5 mL) was added N-ethyldiisopropylamine (66.9 μL, 384 μm) and HATU (58.4 mg, 154 μmol) at room temperature. The mixture stirred at room temperature for 3 hours. The mixture was diluted with EtOAc, washed with saturated aqueous NH₄Cl, washed with water, washed with brine, dried over anhydrous Na₂SO₄, and concentrated in vacuo. The crude residue was purified by reverse-phase chromatography (40-100% MeCN in water with 0.1% FA) to provide (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (18.8 mg, 21.7 μmol) as a white solid. m/z (ESI-pos) M+1=857.6; ¹H NMR (400 MHz, CHLOROFORM-D) δ 11.31 (s, 1H), 7.65-7.54 (m, 2H), 7.52 (d, J=1.6 Hz, 1H), 7.44-7.35 (m, 1H), 7.32 (dd, J=6.1, 2.8 Hz, 1H), 7.26 (s, 1H), 7.24-7.02 (m, 2H), 6.81-6.68 (m, 1H), 6.32 (dd, J=54.1, 3.0 Hz, 2H), 5.84-5.20 (m, 1H), 4.97-4.62 (m, 2H), 4.47 (dd, J=13.8, 5.1 Hz, 1H), 4.27-4.06 (m, 2H), 3.93-3.77 (m, 2H), 3.71-3.36 (m, 1H), 3.23-2.93 (m, 3H), 2.40-2.27 (m, 3H), 1.96-1.37 (m, 10H), 1.37-1.26 (m, 6H), 1.24-1.11 (m, 3H).

### Procedure for Synthesis of Compound 212

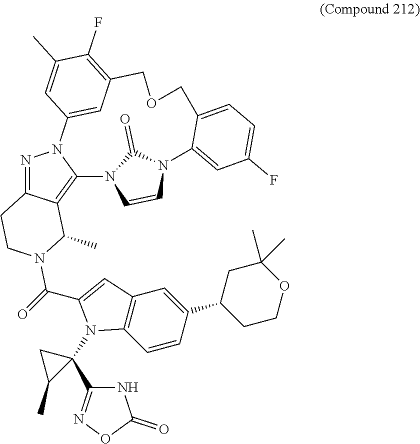

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17,25-difluoro-8,26-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 212 was synthesized following the procedure of Example 211 with (2-bromo-4-fluoro-5-tolyl)methanol being replaced with (2-fluoro-5-iodo-3-tolyl)methanol and 2-(bromomethyl)-1-fluoro-4-iodobenzene being replaced with 2-bromo-4-fluoro-1-[(mesyloxy)methyl]benzene in step A. m/z (ESI-pos) M+1=857.6; ¹H NMR (400 MHz, CHLOROFORM-D) δ 11.38-11.22 (m, 1H), 7.63-7.58 (m, 1H), 7.58-7.42 (m, 3H), 7.29-7.25 (m, 1H), 7.23-7.11 (m, 2H), 7.11-7.00 (m, 1H), 6.80-6.69 (m, 1H), 6.46-5.87 (m, 2H), 5.85-5.23 (m, 1H), 4.96-4.62 (m, 2H), 4.47 (dd, J=13.8, 5.0 Hz, 1H), 4.32-4.08 (m, 2H), 3.93-3.78 (m, 2H), 3.70-3.35 (m, 1H), 3.21-2.96 (m, 3H), 2.37-2.30 (m, 3H), 1.96-1.37 (m, 10H), 1.32 (d, J=23.9 Hz, 6H), 1.17 (dd, J=29.8, 6.0 Hz, 3H).

### Procedure for Synthesis of Compound 213

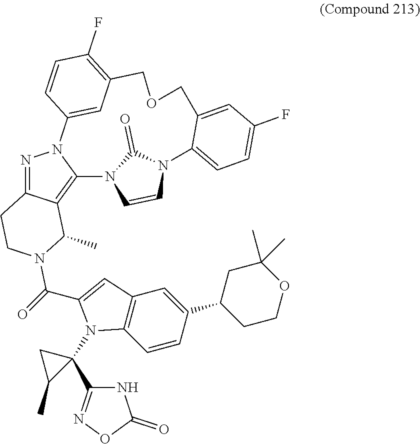

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 213 was synthesized following the procedure of Example 211, with (2-bromo-4-fluoro-5-tolyl)methanol being replaced with (2-bromo-5-fluorophenyl)methanol in step A. m/z (ESI-pos) M+1=843.6; ¹H NMR (400 MHz, CHLOROFORM-D) δ 11.41-11.19 (m, 1H), 7.67-7.54 (m, 2H), 7.52 (d, J=1.6 Hz, 1H), 7.48-7.32 (m, 1H), 7.32-7.23 (m, 3H), 7.23-7.11 (m, 2H), 6.80-6.67 (m, 1H), 6.45-5.87 (m, 2H), 5.85-5.20 (m, 1H), 4.96-4.63 (m, 2H), 4.52-4.42 (m, 1H), 4.30-4.05 (m, 2H), 3.93-3.76 (m, 2H), 3.71-3.33 (m, 1H), 3.24-2.92 (m, 3H), 1.96-1.37 (m, 10H), 1.37-1.25 (m, 6H), 1.24-1.08 (m, 3H).

### Example 214—Synthesis of Compound 214

### Synthetic Scheme

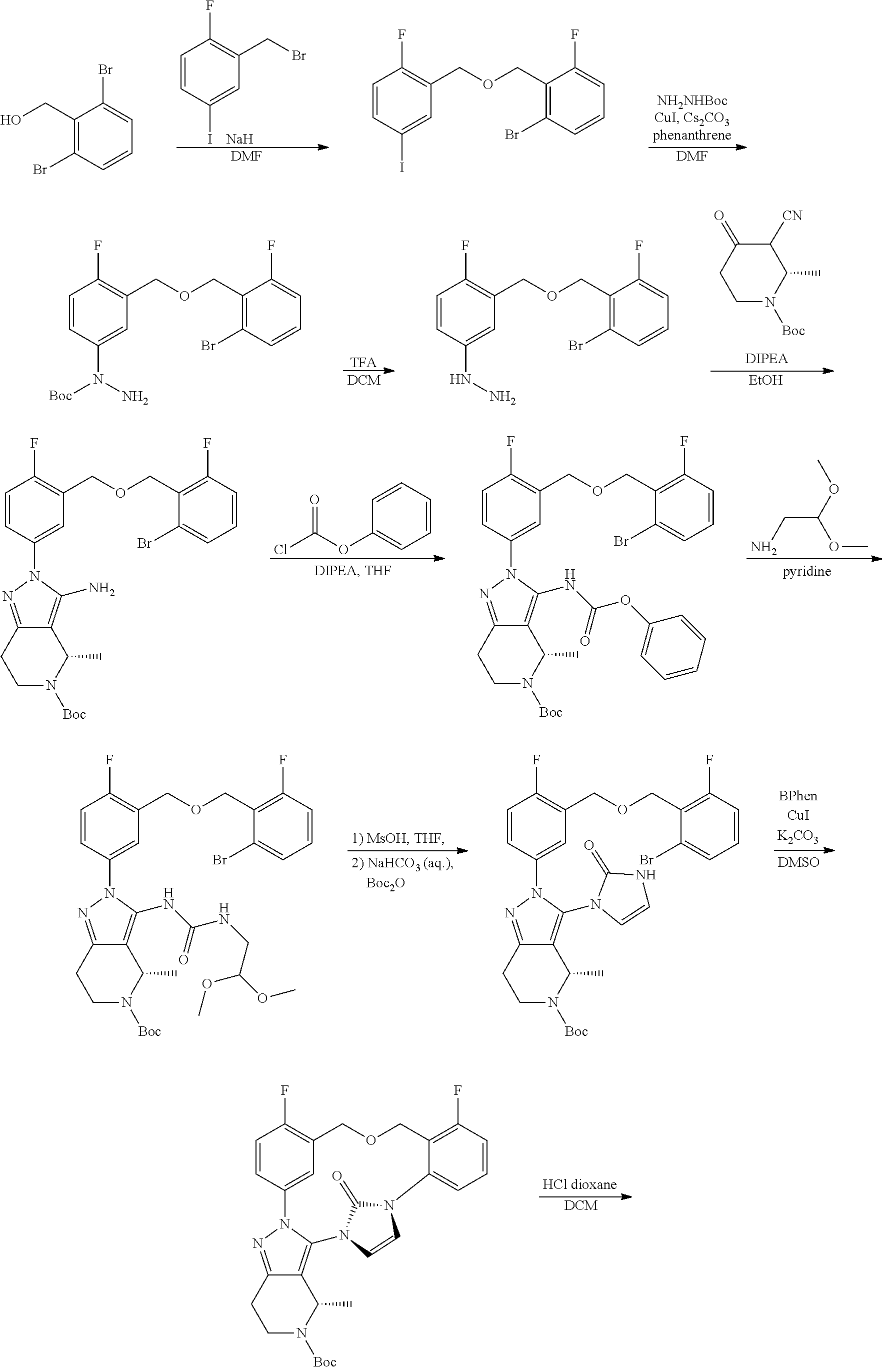

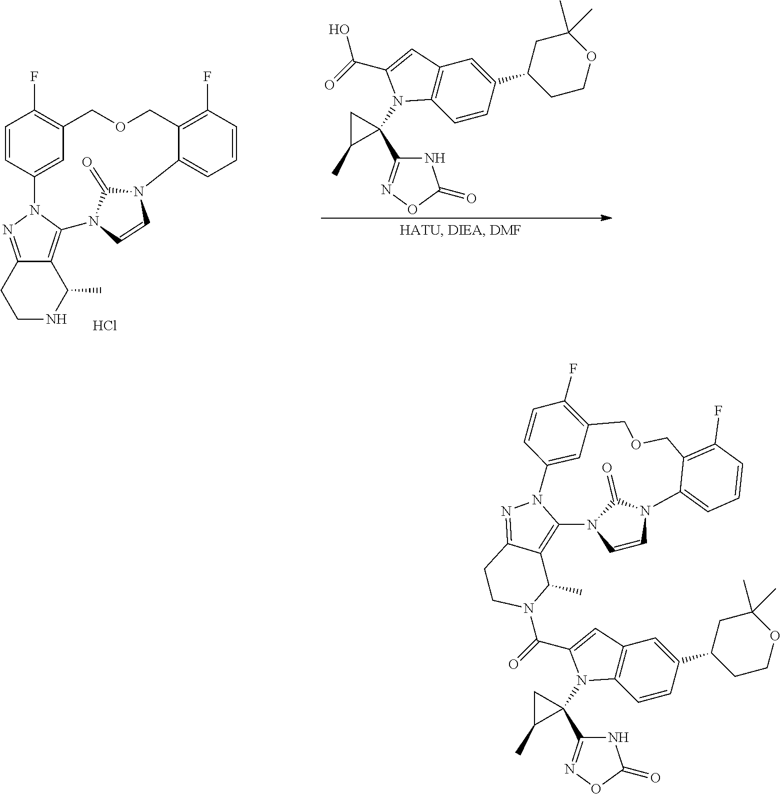

### Procedure for Synthesis of Compound 214

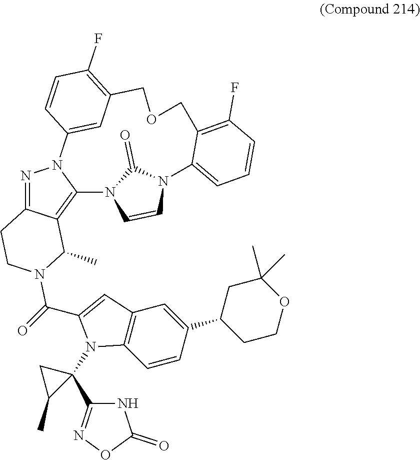

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-19,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of (2-bromo-6-fluorophenyl)methanol (942 mg, 4.59 mmol) in dimethylformamide (15 mL) was added sodium hydride 60% w/w (276 mg, 6.89 mmol) at 0° C. The reaction was stirred at 0° C. for 20 minutes and 2-(bromomethyl)-1- fluoro-4-iodobenzene (1.66 g, 5.28 mmol) was added. The reaction was allowed to warm to room temperature and stirred for 1 hour. The mixture was slowly quenched with saturated aqueous NH₄Cl and extracted with EtOAc. The organics were washed with brine (2×), dried over Na₂SO₄, filtered, and concentrated in vacuo. The crude product was purified by column chromatography (silica gel, 0-15% EtOAc in heptane) to give 1-bromo-3-fluoro-2-{[(2-fluoro-5-iodophenyl)methoxy]methyl}benzene (1.72 g, 3.73 mmol). 1H NMR (400 MHz, CHLOROFORM-D) δ 7.80 (dd, J=6.8, 2.4 Hz, 1H), 7.54 (ddd, J=8.6, 4.9, 2.4 Hz, 1H), 7.39 (dt, J=8.1, 1.1 Hz, 1H), 7.18 (td, J=8.2, 5.9 Hz, 1H), 7.05 (ddd, J=9.4, 8.3, 1.2 Hz, 1H), 6.79 (dd, J=9.7, 8.6 Hz, 1H), 4.75 (d, J=2.4 Hz, 2H), 4.67-4.53 (m, 2H).

Step B: A mixture of 1-bromo-3-fluoro-2-{[(2-fluoro-5-iodophenyl)methoxy]methyl}benzene (1.72 g, 3.92 mmol), tert-butyl carbazate (622 mg, 4.71 mmol), cesium carbonate (1.79 g, 5.49 mmol), copper iodide (74.7 mg, 392 μmol), and 1,10-phenanthroline (283 mg, 1.57 mmol) in DMF (5 mL) was sparged with N₂ for 5 minutes. The reaction mixture was stirred at 80° C. for 10 hours. The reaction mixture was allowed to cool to room temperature, diluted with EtOAc, and filtered through a pad of celite. The filtrate was washed with brine (2×). The organic layer was dried over Na₂SO₄ and concentrated in vacuo. The crude residue was purified by column chromatography (silica gel, 10-50% EtOAc in heptane) to afford tert-butyl 3-(3-{[(2-bromo-6-fluorophenyl)methoxy]methyl}-4-fluorophenyl)carbazate (728 mg, 1.64 mmol). m/z (ESI-pos) M+1=387.1, 389.1 (loss of tBu).

Step C: Trifluoroacetic acid (1.26 mL, 16.4 mmol) was added to a stirred solution of tert-butyl 2-(3-{[(2-bromo-6-fluorophenyl)methoxy]methyl}-4-fluorophenyl)carbazate (728 mg, 1.64 mmol) in dichloromethane (5.5 mL) at room temperature. The reaction was stirred at room temperature for 2.5 hours. The reaction was quenched with sat. aq. Na₂CO₃, extracted with DCM, washed with brine, dried over Na₂SO₄, filtered, and concentrated in vacuo to provide (3-{[(2-bromo-6-fluorophenyl)methoxy]methyl}-4-fluorophenyl)hydrazine (563 mg, 1.64 mmol). m/z (ESI-pos) M+1=343.1, 345.1.

Step D: Tert-butyl(2S)-3-cyano-2-methyl-4-oxo-1-piperidinecarboxylate (469 mg, 1.97 mmol) and N-ethyldiisopropylamine (429 μL, 2.46 mmol) were added to a stirred solution of (3-{[(2-bromo-6-fluorophenyl)methoxy]methyl}-4-fluorophenyl)hydrazine (563 mg, 1.64 mmol) in ethanol (5.5 mL) at room temperature. The mixture was stirred at 80° C. overnight. The reaction mixture was allowed to cool to room temperature, diluted with water, and extracted with DCM (2×). The organic layers were dried over anhydrous Na₂SO₄ and concentrated in vacuo. The crude compound was purified by column chromatography (silica, 10-50% EtOAc/heptane) to afford tert-butyl (S)-3-amino-2-(3-{[(2-bromo-6-fluorophenyl)methoxy]methyl}-4-fluorophenyl)-4-methyl-1,2,5-triaza-2,4,6,7-tetrahydro-5H-indene-5-carboxylate (285 mg, 506 μmol). m/z (ESI-pos) M+1=563.3, 565.3.

Step E: To a stirred solution of tert-butyl (S)-3-amino-2-(3-{[(2-bromo-6-fluorophenyl)methoxy]methyl}-4-fluorophenyl)-4-methyl-1,2,5-triaza-2,4,6,7-tetrahydro-5H-indene-5-carboxylate (285 mg, 506 μmol) in tetrahydrofuran (2.5 mL) was added N-ethyldiisopropylamine (264 μL, 1.52 mmol) followed by benzenyl chloroformate (127 μL, 1.01 mmol). The reaction mixture was stirred at room temperature for 30 minutes. The reaction mixture was concentrated in vacuo to give crude tert-butyl (S)-2-(3-{[(2-bromo-6-fluorophenyl)methoxy]methyl}-4-fluorophenyl)-4-methyl-3-[phenyl(oxycarbonylamino)]-1,2,5-triaza-2,4,6,7-tetrahydro-5H-indene-5-carboxylate which was used directly in the next step. m/z (ESI-pos) M+1=683.4, 685.3.

Step F: 2,2-Dimethoxyethylamine (221 μL, 2.02 mmol) was added to a stirred solution of crude tert-butyl (S)-2-(3-{[(2-bromo-6-fluorophenyl)methoxy]methyl}-4-fluorophenyl)-4-methyl-3-[phenyl(oxycarbonylamino)]-1,2,5-triaza-2,4,6,7-tetrahydro-5H-indene-5-carboxylate in pyridine (2.53 mL). The mixture was stirred at room temperature for 30 minutes and concentrated in vacuo. The crude product was purified by column chromatography (silica, 10-60% EtOAc in heptane) to give tert-butyl (S)-2-(3-{[(2-bromo-6-fluorophenyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (263 mg, 378 μmol). m/z (ESI-pos) M+1=694.4, 696.4.

Step G: To a solution of tert-butyl (S)-2-(3-{[(2-bromo-6-fluorophenyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (263 mg, 378 μmol) in tetrahydrofuran (2.25 mL) was added methanesulfonic acid (123 μL, 1.89 mmol). The mixture was stirred at 55° C. for 1 hour then allowed to cool to ambient temperature. Saturated aqueous NaHCO₃ (2.25 mL) and tert-butyl-tert-butyl(oxycarbonyloxy) formylate (165 mg, 756 μmol) were added, and the mixture was stirred at room temperature for 30 minutes. The mixture was extracted with DCM, dried over anhydrous Na₂SO₄, and concentrated in vacuo. The crude residue was purified by column chromatography (silica gel, 10-80% EtOAc in heptane) to give tert-butyl (S)-2-(3-{[(2-bromo-6-fluorophenyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (226 mg, 359 μmol). m/z (ESI-pos) M+1=630.3, 632.4.

Step H: A mixture of tert-butyl (S)-2-(3-{[(2-bromo-6-fluorophenyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (226 mg, 359 μmol), bathophenanthroline (119 mg, 359 μmol), and dipotassium carbonate (124 mg, 897 μmol) in dimethyl sulfoxide (12 mL) was sparged with N₂ for 5 minutes. Copper iodide (51.2 mg, 269 μmol) was added, and the mixture was sparged with N₂ for 10 minutes. The reaction was stirred at 130° C. for 40 hours. The reaction mixture was allowed to cool to room temperature then filtered through a pad of Celite with EtOAc. The filtrate was washed with saturated aqueous NH₄Cl and washed with brine. The organic layer was dried over sodium sulfate and concentrated in vacuo. The crude residue was purified by column chromatography (silica gel, 5-40% EtOAc in heptane) to give tert-butyl (8S,10M)-19,25-difluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (29.9 mg, 54.4 μmol). m/z (ESI-pos) M+1=550.4.

Step I: To a solution of tert-butyl (8S,10M)-19,25-difluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (29.9 mg, 54.4 μmol) in dichloromethane (1 mL) was added 4M HCl in dioxane (1 mL) at room temperature. The reaction was stirred at room temperature for 40 minutes. The reaction was concentrated in vacuo to provide (8S,10M)-19,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (26.4 mg, 54.3 μmol). m/z (ESI-pos) M+1=450.3.

Step J: To a solution of (8S,10M)-19,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (26.4 mg, 54.3 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (29.1 mg, 70.6 μmol) in dimethylformamide (1.1 mL) was added N-ethyldiisopropylamine (47.3 μL, 272 μmol) and HATU (41.3 mg, 109 μmol) at room temperature. The mixture was stirred at room temperature overnight. The mixture was diluted with EtOAc, washed with saturated aqueous NH₄Cl, washed with brine, dried over anhydrous Na₂SO₄, and concentrated in vacuo. The crude residue was purified by reverse-phase chromatography (40-100% MeCN in water with 0.1% FA) to provide (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-19,25-difluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (28.2 mg, 33.2 μmol) as a white solid. m/z (ESI-pos) M+1=843.6; ¹H NMR (400 MHz, CHLOROFORM-D) δ 11.38-11.22 (m, 1H), 7.65-7.41 (m, 4H), 7.36 (dd, J=6.1, 2.8 Hz, 1H), 7.32-7.13 (m, 5H), 6.81-6.66 (m, 1H), 6.43-5.85 (m, 2H), 5.84-5.21 (m, 1H), 4.98-4.64 (m, 2H), 4.53-4.33 (m, 1H), 4.28-4.10 (m, 1H), 3.94-3.75 (m, 2H), 3.72-3.35 (m, 1H), 3.22-2.94 (m, 3H), 1.97-1.45 (m, 10H), 1.37-1.26 (m, 6H), 1.24-1.10 (m, 3H).

### Example 215—Synthesis of Compound 215

### Synthetic Scheme

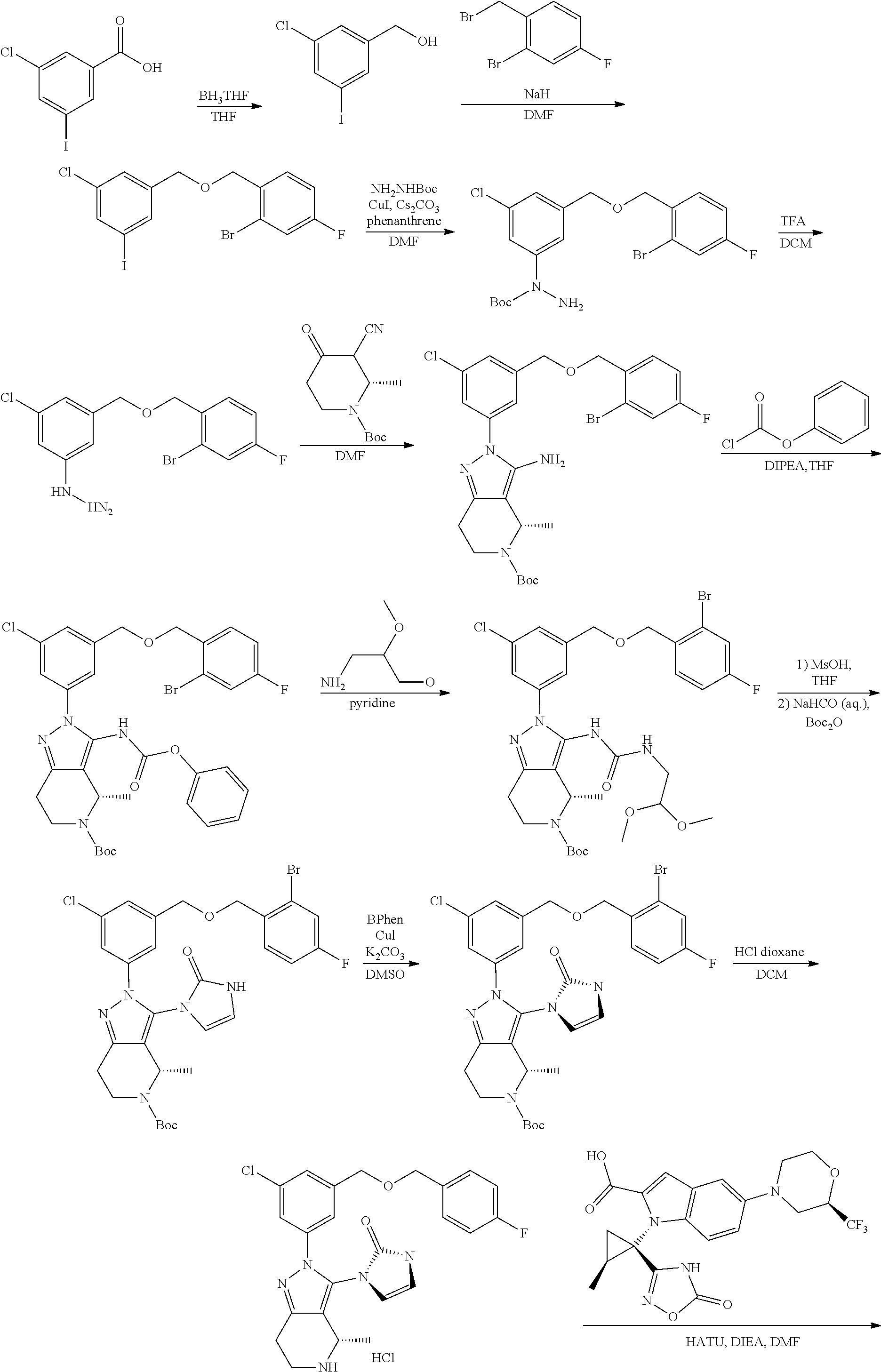

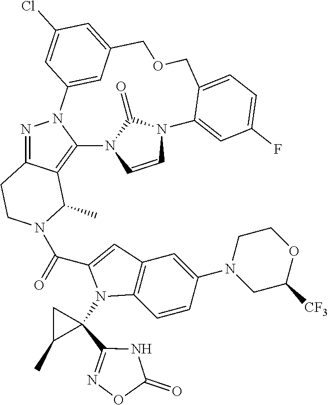

### Procedure for Synthesis of Compound 215

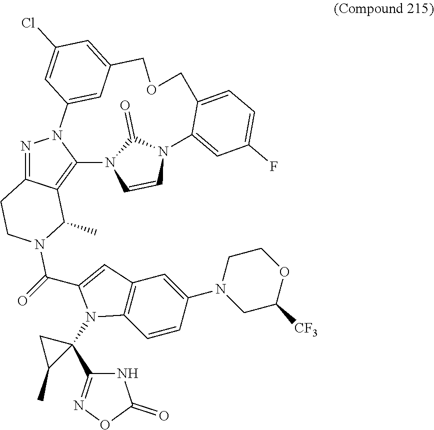

### (8S,10M)-26-chloro-17-fluoro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: 1M Borane in THF (170 mL, 170 mmol) was added dropwise to a stirred solution of 3-chloro-5-iodobenzoic acid (24 g, 85.1 mmol) in tetrahydrofuran (100 mL) at 0° C. The reaction was stirred at 0° C. for 30 minutes and at room temperature for 2 hours. The reaction mixture was cooled 0° C., quenched with saturated aqueous NaHCO₃, diluted with EtOAc, and washed with brine (2×). The organic layer was dried over Na₂SO₄ and concentrated in vacuo to give (3-chloro-5-iodophenyl)methanol (20.7 g, 77.1 mmol) as a white solid.

Step B: To a solution of (3-chloro-5-iodophenyl)methanol (3 g, 11.2 mmol) in dimethylformamide (37 mL) was added sodium hydride 60% w/w (670 mg, 16.8 mmol) at 0° C. The mixture was stirred at 0° C. for 30 minutes. 2-Bromo-1-(bromomethyl)-4-fluorobenzene (3.29 g, 12.3 mmol) was added and the reaction was stirred at 0° C. for 30 minutes. The reaction was allowed to warm to room temperature and stirred for 45 minutes. The reaction mixture was quenched with saturated aqueous NH₄Cl, diluted with EtOAc, washed with brine (2×). The organic layer was dried over Na₂SO₄, filtered, and concentrated in vacuo. The crude product was purified by column chromatography (silica gel, 0-15% EtOAc in heptane) to give 2-bromo-1-{[(3-chloro-5-iodophenyl)methoxy]methyl}-4-fluorobenzene (4.21 g, 9.24 mmol).

Step C: A mixture of 2-bromo-1-{[(3-chloro-5-iodophenyl)methoxy]methyl}-4-fluorobenzene (4.21 g, 9.24 mmol), tert-butyl carbazate (1.47 g, 11.1 mmol), cesium carbonate (4.22 g, 12.9 mmol), copper iodide (176 mg, 924 μmol), and 1,10-phenanthroline (666 mg, 3.7 mmol) in DMF (12 mL) was sparged with N₂ for 10 minutes. The reaction mixture was stirred at 80° C. for 8 hours. The reaction mixture was allowed to cool to room temperature, diluted with EtOAc, and filtered through a pad of celite. The filtrate was washed with brine (2×). The organic layer was dried over Na₂SO₄ and concentrated in vacuo. The crude residue was purified by column chromatography (silica gel, 0-50% EtOAc in heptane) to afford tert-butyl 3-(5-{[(2-bromo-4-fluorophenyl)methoxy]methyl}-3-chlorophenyl)carbazate (3.48 g, 7.57 mmol). m/z (ESI-pos) M+1=359.1, 361.1 (loss of tBu).

Step D: Trifluoroacetic acid (5.79 mL, 75.7 mmol) was added to a stirred solution of tert-butyl 2-(5-{[(2-bromo-4-fluorophenyl)methoxy]methyl}-3-chlorophenyl)carbazate (3.48 g, 7.57 mmol) in dichloromethane (25 mL) at room temperature. The reaction was stirred at room temperature for 3 hours. The reaction was quenched with sat. aq. Na₂CO₃, extracted with DCM, washed with brine, dried over Na₂SO₄, filtered, and concentrated in vacuo to provide provide (5-{[(2-bromo-4-fluorophenyl)methoxy]methyl}-3-chlorophenyl)hydrazine (2.72 g, 7.56 mmol). m/z (ESI-pos) M+1=359.1, 361.1

Step E: Tert-butyl(2S)-3-cyano-2-methyl-4-oxo-1-piperidinecarboxylate (1.8 g, 7.56 mmol) was added to a stirred solution of (5-{[(2-bromo-4-fluorophenyl)methoxy]methyl}-3-chlorophenyl)hydrazine (2.72 g, 7.56 mmol) in ethanol (15 mL) at room temperature. The mixture was stirred at 80° C. overnight. The reaction mixture was allowed to cool to room temperature and concentrated in vacuo. The crude compound was purified by column chromatography (silica, 10-50% EtOAc/heptane) to afford tert-butyl (S)-3-amino-2-(5-{[(2-bromo-4-fluorophenyl)methoxy]methyl}-3-chlorophenyl)-4-methyl-1,2,5-triaza-2,4,6,7-tetrahydro-5H-indene-5-carboxylate (2.77 g, 4.78 mmol). m/z (ESI-pos) M+1=579.3, 581.3.

Step F: To a stirred solution of tert-butyl (S)-3-amino-2-(5-{[(2-bromo-4-fluorophenyl)methoxy]methyl}-3-chlorophenyl)-4-methyl-1,2,5-triaza-2,4,6,7-tetrahydro-5H-indene-5-carboxylate (2.77 g, 4.78 mmol) in tetrahydrofuran (24 mL) was added N-ethyldiisopropylamine (2.5 mL, 14.3 mmol) followed by benzenyl chloroformate (1.2 mL, 9.55 mmol). The reaction mixture was stirred at room temperature for 45 minutes. The reaction mixture was concentrated in vacuo to give crude tert-butyl (S)-2-(5-{[(2-bromo-4-fluorophenyl)methoxy]methyl}-3-chlorophenyl)-4-methyl-3-[phenyl(oxycarbonylamino)]-1,2,5-triaza-2,4,6,7-tetrahydro-5H-indene-5-carboxylate which was used directly in the next step. m/z (ESI-pos) M+1=699.3, 701.3.

Step G: 2,2-Dimethoxyethylamine (2.6 mL, 23.9 mmol) was added to a stirred solution of crude tert-butyl (S)-2-(5-{[(2-bromo-4-fluorophenyl)methoxy]methyl}-3-chlorophenyl)-4-methyl-3-[phenyl(oxycarbonylamino)]-1,2,5-triaza-2,4,6,7-tetrahydro-5H-indene-5-carboxylate in pyridine (24 mL). The mixture was stirred at room temperature for 30 minutes and concentrated in vacuo. The residue was diluted with EtOAc and washed with sat. aq. NaHCO₃. The organic layer was dried over Na₂SO₄, filtered, and concentrated in vacuo. The crude product was purified by column chromatography (silica, 10-80% EtOAc in heptane) to give tert-butyl (S)-2-(5-{[(2-bromo-4-fluorophenyl)methoxy]methyl}-3-chlorophenyl)-3-[3-(2,2-dimethoxyethyl)ureido]-4-methyl-1,2,5-triaza-2,4,6,7-tetrahydro-5H-indene-5-carboxylate (2.63 g, 3.7 mmol). m/z (ESI-pos) M+1=710.3, 712.3.

Step H: To a solution of tert-butyl (S)-2-(5-{[(2-bromo-4-fluorophenyl)methoxy]methyl}-3-chlorophenyl)-3-[3-(2,2-dimethoxyethyl)ureido]-4-methyl-1,2,5-triaza-2,4,6,7-tetrahydro-5H-indene-5-carboxylate (2.63 g, 3.7 mmol) in tetrahydrofuran (37 mL) was added methanesulfonic acid (1.2 mL, 18.5 mmol). The mixture was stirred at 55° C. for 1 hour then allowed to cool to ambient temperature. Saturated aqueous NaHCO₃ (37 mL) and tert-butyl-tert-butyl(oxycarbonyloxy) formylate (1.61 g, 7.4 mmol) were added, and the mixture was stirred at room temperature for 30 minutes. The mixture was extracted with DCM, dried over anhydrous Na₂SO₄, and concentrated in vacuo. The crude residue was purified by column chromatography (silica gel, 10-80% EtOAc in heptane) to give tert-butyl (S)-2-(5-{[(2-bromo-4-fluorophenyl)methoxy]methyl}-3-chlorophenyl)-4-methyl-3-(2-oxo-2,3-dihydro-1H-imidazol-1-yl)-1,2,5-triaza-2,4,6,7-tetrahydro-5H-indene-5-carboxylate (2.14 g, 3.31 mmol). m/z (ESI-pos) M+1=646.3, 648.3.

Step I: A solution of tert-butyl (S)-2-(5-{[(2-bromo-4-fluorophenyl)methoxy]methyl}-3-chlorophenyl)-4-methyl-3-(2-oxo-2,3-dihydro-1H-imidazol-1-yl)-1,2,5-triaza-2,4,6,7-tetrahydro-5H-indene-5-carboxylate (0.5 g, 773 μmol), copper iodide (110 mg, 580 μmol), bathophenanthroline (257 mg, 773 μmol), and dipotassium carbonate (267 mg, 1.93 mmol) in dimethyl sulfoxide (26 mL) was sparged with N₂ for 15 minutes. The reaction was stirred at 130° C. for 40 hours. The reaction mixture was allowed to cool to room temperature then filtered through a pad of Celite with EtOAc. The filtrate was washed with saturated aqueous NH₄Cl and washed with brine. The organic layer was dried over sodium sulfate and concentrated in vacuo. The crude residue was purified by column chromatography (silica gel, 5-40% EtOAc in heptane) to give tert-butyl (8S,10M)-26-chloro-17-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (59.2 mg, 105 μmol). m/z (ESI-pos) M+1=566.3.

Step J: 4M HCl in dioxane (0.5 mL) was added to a stirred solution of tert-butyl (8S,10M)-26-chloro-17-fluoro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (19.7 mg, 34.8 μmol) in dichloromethane (0.5 mL) at room temperature. The solution was stirred at room temperature for 1 hour and concentrated in vacuo to provide (8S,10M)-26-chloro-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (17.5 mg, 34.8 μmol). m/z (ESI-pos) M+1=466.2.

Step K: To a solution of (8S,10M)-26-chloro-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (17.5 mg, 34.8 μmol) and 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid (20.5 mg, 45.3 μmol) in dimethylformamide (1 mL) was added N-ethyldiisopropylamine (24.3 μL, 139 μmol) and HATU (26.5 mg, 69.7 μmol) at room temperature. The mixture was stirred at room temperature overnight. The mixture was diluted with EtOAc, washed with saturated aqueous NH₄Cl, washed with brine (2×), dried over anhydrous Na₂SO₄, and concentrated in vacuo. The crude residue was purified by reverse-phase chromatography (40-100% MeCN in water with 0.1% FA) to provide (8S,10M)-26-chloro-17-fluoro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (14.1 mg, 15.5 μmol) as a white solid. m/z (ESI-pos) M+1=900.5; ¹H NMR (400 MHz, CHLOROFORM-D) δ 11.34-11.18 (m, 1H), 7.74-7.66 (m, 1H), 7.62-7.55 (m, 1H), 7.53-7.46 (m, 1H), 7.22-7.01 (m, 6H), 6.79-6.64 (m, 1H), 6.56-5.90 (m, 2H), 5.87-5.22 (m, 1H), 4.98-4.44 (m, 3H), 4.38-4.00 (m, 4H), 3.91 (td, J=11.4, 2.6 Hz, 1H), 3.73-3.50 (m, 2H), 3.48-3.29 (m, 1H), 3.23-2.80 (m, 4H), 1.95-1.82 (m, 1H), 1.81-1.25 (m, 5H), 1.24-1.10 (m, 3H).

### Example 216—Synthesis of Compound 216

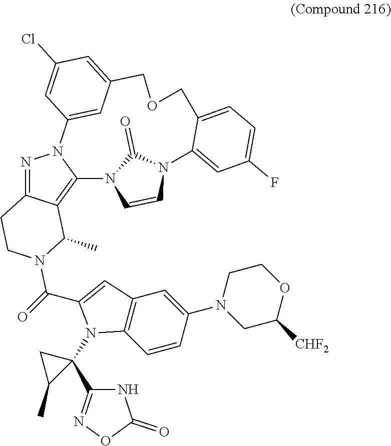

### (8S,10M)-26-chloro-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 216 was synthesized following the procedure of Example 215, with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid being replaced with 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid in step K. m/z (ESI-pos) M+1=882.5; ¹H NMR (400 MHz, CHLOROFORM-D) δ 11.33-11.20 (M, 1H), 7.74-7.66 (M, 1H), 7.62-7.54 (M, 1H), 7.53-7.45 (M, 1H), 7.23-7.00 (M, 6H), 6.78-6.63 (M, 1H), 6.55-5.21 (M, 4H), 4.97-4.44 (M, 3H), 4.37-4.20 (M, 1H), 4.15-3.83 (M, 4H), 3.63 (TD, J=13.3, 4.2 HZ, 1H), 3.51 (D, J=11.5 HZ, 1H), 3.37 (D, J=11.9 HZ, 1H), 3.22-2.98 (M, 2H), 2.92 (TD, J=11.6, 3.3 HZ, 1H), 2.82 (T, J=10.9 HZ, 1H), 1.95-1.82 (M, 1H), 1.81-1.24 (M, 5H), 1.24-1.10 (M, 3H). m/z (ESI-pos) M+1=882.5

### Example 217—Synthesis of Compound 217

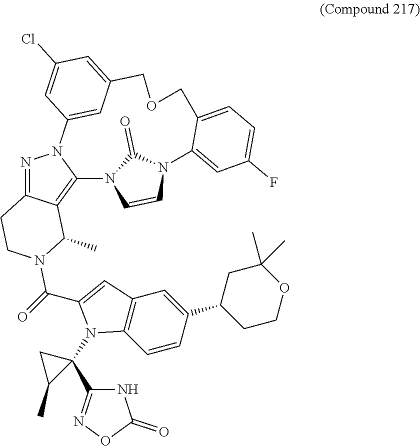

### (8S,10M)-26-chloro-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17-fluoro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 217 was synthesized following the procedure of Example 215, with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid being replaced with 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid in step K. ¹H NMR (400 MHz, CHLOROFORM-D) A 11.38-11.17 (M, 1H), 7.74-7.65 (M, 1H), 7.63-7.57 (M, 1H), 7.57-7.45 (M, 2H), 7.29-7.25 (M, 1H), 7.21-7.14 (M, 3H), 7.12-7.01 (M, 1H), 6.81-6.69 (M, 1H), 6.56-5.90 (M, 2H), 5.88-5.24 (M, 1H), 4.97-4.42 (M, 3H), 4.37-4.00 (M, 2H), 3.93-3.76 (M, 2H), 3.70-3.34 (M, 1H), 3.23-2.93 (M, 3H), 1.96-1.38 (M, 10H), 1.32 (D, J=23.9 HZ, 6H), 1.24-1.08 (M, 3H). m/z (ESI-pos) M+1=859.5

### Example 218—Synthesis of Compound 218

### Synthetic Scheme

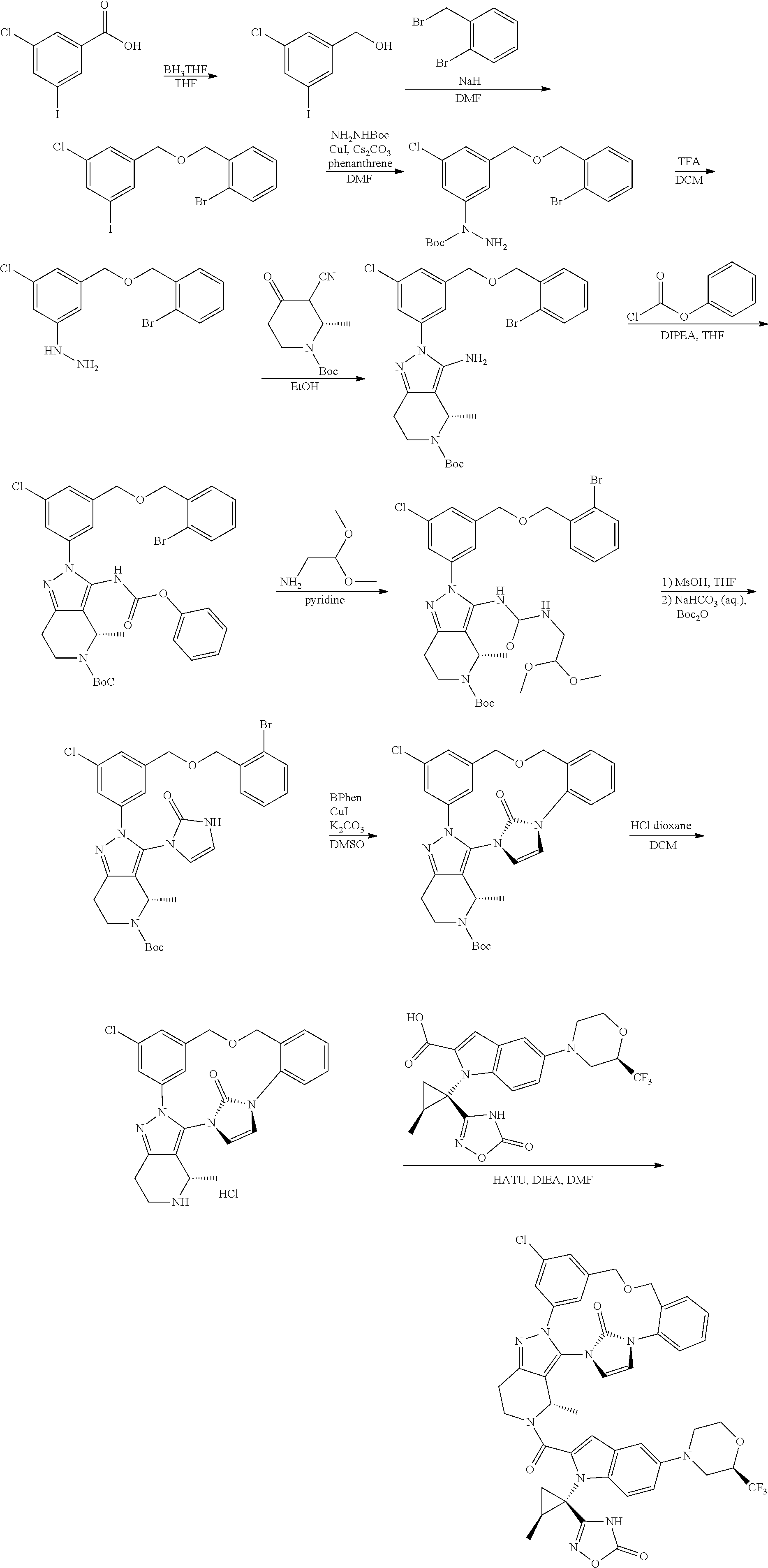

### Procedure for Synthesis of Compound 218

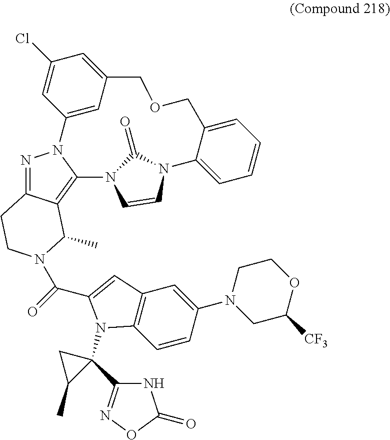

### (8S,10M)-26-chloro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: 1M Borane in THF (170 mL, 170 mmol) was added dropwise to a stirred solution of 3-chloro-5-iodobenzoic acid (24 g, 85.1 mmol) in tetrahydrofuran (100 mL) at 0° C. The reaction was stirred at 0° C. for 30 minutes and at room temperature for 2 hours. The reaction mixture was cooled 0° C., quenched with saturated aqueous NaHCO₃, diluted with EtOAc, and washed with brine (2×). The organic layer was dried over Na₂SO₄ and concentrated in vacuo to give (3-chloro-5-iodophenyl)methanol (20.7 g, 77.1 mmol) as a white solid.

Step B: Sodium hydride 60% w/w (1.12 g, 27.9 mmol) was added to a stirred solution of (3-chloro-5-iodophenyl)methanol (5 g, 18.6 mmol) in dimethylformamide (62 mL) at 0° C. The mixture was stirred at 0° C. for 15 minutes and o-bromo (bromomethyl)benzene (5.12 g, 20.5 mmol) was added. The reaction was stirred at 0° C. for 30 minutes and at room temperature for 1 hour. The reaction mixture was quenched with saturated aqueous NH₄Cl at 0° C., diluted with EtOAc, and washed with brine (2×). The organic layer was dried over Na₂SO₄, filtered, and concentrated in vacuo. The crude product was purified by column chromatography (silica gel, 0-15% EtOAc in heptane) to give 5-{[(o-bromophenyl)methoxy]methyl}-1-chloro-3-iodobenzene (7.76 g, 17.7 mmol).

Step C: A mixture of 5-{[(o-bromophenyl)methoxy]methyl}-1-chloro-3-iodobenzene (7.76 g, 17.7 mmol), tert-butyl carbazate (2.81 g, 21.3 mmol), cesium carbonate (8.09 g, 24.8 mmol), copper iodide (338 mg, 1.77 mmol), and 1,10-phenanthroline (1.28 g, 7.09 mmol) in DMF (22.5 mL) was sparged with N₂ for 5 minutes. The reaction mixture was stirred at 80° C. overnight. The reaction mixture was allowed to cool to room temperature, diluted with EtOAc, and filtered through a pad of celite. The filtrate was washed with brine (2×). The organic layer was dried over Na₂SO₄ and concentrated in vacuo. The crude residue was purified by column chromatography (silica gel, 10-40% EtOAc in heptane) to afford tert-butyl 3-(5-{[(o-bromophenyl)methoxy]methyl}-3-chlorophenyl)carbazate (6.34 g, 14.4 mmol). m/z (ESI-pos) M+1=341.1, 343.0 (loss of tBu).

Step D: Trifluoroacetic acid (11 mL, 144 mmol) was added to a stirred solution of tert-butyl 2-(5-{[(o-bromophenyl)methoxy]methyl}-3-chlorophenyl)carbazate (6.34 g, 14.4 mmol) in dichloromethane (48 mL) at room temperature. The reaction was stirred at room temperature for 3 hours. The reaction was quenched with sat. aq. Na₂CO₃, extracted with DCM, washed with brine, dried over Na₂SO₄, filtered, and concentrated in vacuo to provide (5-{[(o-bromophenyl)methoxy]methyl}-3-chlorophenyl)hydrazine (4.91 g, 14.4 mmol). m/z (ESI-pos) M+1=341.0, 343.1.

Step E: Tert-butyl(2S)-3-cyano-2-methyl-4-oxo-1-piperidinecarboxylate (4.11 g, 17.2 mmol) and N-ethyldiisopropylamine (2.5 mL, 14.4 mmol) were added to a stirred solution of (5-{[(o-bromophenyl)methoxy]methyl}-3-chlorophenyl)hydrazine (4.91 g, 14.4 mmol) in ethanol (43 mL) at room temperature. The mixture was stirred at 80° C. overnight. The reaction mixture was allowed to cool to room temperature, diluted with water, and extracted with DCM (2×). The organic layers were dried over anhydrous Na₂SO₄ and concentrated in vacuo. The crude compound was purified by column chromatography (silica, 10-50% EtOAc/heptane) to afford tert-butyl (S)-3-amino-2-(5-{[(o-bromophenyl)methoxy]methyl}-3-chlorophenyl)-4-methyl-1,2,5-triaza-2,4,6,7-tetrahydro-5H-indene-5-carboxylate (4.5 g, 8.01 mmol). m/z (ESI-pos) M+1=561.3, 563.3.

Step F: To a stirred solution of tert-butyl (S)-3-amino-2-(5-{[(o-bromophenyl)methoxy]methyl}-3-chlorophenyl)-4-methyl-1,2,5-triaza-2,4,6,7-tetrahydro-5H-indene-5-carboxylate (4.5 g, 8.01 mmol) in tetrahydrofuran (40 mL) was added N-ethyldiisopropylamine (4.18 mL, 24 mmol) followed by benzenyl chloroformate (2.01 mL, 16 mmol). The reaction mixture was stirred at room temperature for 30 minutes. The reaction mixture was concentrated in vacuo to give crude tert-butyl (S)-2-(5-{[(o-bromophenyl)methoxy]methyl}-3-chlorophenyl)-4-methyl-3-[phenyl(oxycarbonylamino)]-1,2,5-triaza-2,4,6,7-tetrahydro-5H-indene-5-carboxylate which was used directly in the next step. m/z (ESI-pos) M+1=681.3, 683.3.

Step G: 2,2-Dimethoxyethylamine (3.49 mL, 32 mmol) was added to a stirred solution of crude tert-butyl (S)-2-(5-{[(o-bromophenyl)methoxy]methyl}-3-chlorophenyl)-4-methyl-3-[phenyl(oxycarbonylamino)]-1,2,5-triaza-2,4,6,7-tetrahydro-5H-indene-5-carboxylate in pyridine (40 mL). The mixture was stirred at room temperature for 30 minutes and concentrated in vacuo. The crude product was purified by column chromatography (silica, 10-80% EtOAc in heptane) to give tert-butyl (S)-2-(5-{[(o-bromophenyl)methoxy]methyl}-3-chlorophenyl)-3-[3-(2,2-dimethoxyethyl)ureido]-4-methyl-1,2,5-triaza-2,4,6,7-tetrahydro-5H-indene-5-carboxylate (4.29 g, 6.19 mmol). m/z (ESI-pos) M+1=692.3, 694.3.

Step H: To a solution of tert-butyl (S)-2-(5-{[(o-bromophenyl)methoxy]methyl}-3-chlorophenyl)-3-[3-(2,2-dimethoxyethyl)ureido]-4-methyl-1,2,5-triaza-2,4,6,7-tetrahydro-5H-indene-5-carboxylate (4.29 g, 6.19 mmol) in tetrahydrofuran (62 mL) was added methanesulfonic acid (2.01 mL, 31 mmol). The mixture was stirred at 55° C. for 1 hour then allowed to cool to ambient temperature. Saturated aqueous NaHCO₃ (62 mL) and tert-butyl-tert-butyl(oxycarbonyloxy) formylate (2.7 g, 12.4 mmol) were added, and the mixture was stirred at room temperature for 30 minutes. The mixture was extracted with DCM, dried over anhydrous Na₂SO₄, and concentrated in vacuo. The crude residue was purified by column chromatography (silica gel, 10-80% EtOAc in heptane) to give tert-butyl (S)-2-(5-{[(o-bromophenyl)methoxy]methyl}-3-chlorophenyl)-4-methyl-3-(2-oxo-2,3-dihydro-1H-imidazol-1-yl)-1,2,5-triaza-2,4,6,7-tetrahydro-5H-indene-5-carboxylate (3.89 g, 6.18 mmol). m/z (ESI-pos) M+1=628.3, 630.3.

Step I: A mixture of tert-butyl (S)-2-(5-{[(o-bromophenyl)methoxy]methyl}-3-chlorophenyl)-4-methyl-3-(2-oxo-2,3-dihydro-1H-imidazol-1-yl)-1,2,5-triaza-2,4,6,7-tetrahydro-5H-indene-5-carboxylate (0.5 g, 795 μmol), bathophenanthroline (264 mg, 795 μmol), and dipotassium carbonate (275 mg, 1.99 mmol) in dimethyl sulfoxide (27 mL) was sparged with N₂ for 5 minutes. Copper iodide (114 mg, 596 μmol) was added, and the mixture was sparged with N₂ for 10 minutes. The reaction was stirred at 120° C. for 40 hours. The reaction mixture was allowed to cool to room temperature then filtered through a pad of Celite with EtOAc. The filtrate was washed with saturated aqueous NH₄Cl and washed with brine. The organic layer was dried over sodium sulfate and concentrated in vacuo. The crude residue was purified by column chromatography (silica gel, 5-40% EtOAc in heptane) to give tert-butyl (8S,10M)-26-chloro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (33.9 mg, 61.9 μmol). m/z (ESI-pos) M+1=548.3.

Step J: 4M HCl in dioxane (0.5 mL) was added to a stirred solution of tert-butyl (8S,10M)-26-chloro-8-methyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (28.5 mg, 52 μmol) in dichloromethane (0.5 mL) at room temperature. The solution was stirred at room temperature for 2 hours and concentrated in vacuo to provide (8S,10M)-26-chloro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (25.2 mg, 52 μmol). m/z (ESI-pos) M+1=448.3.

Step K: To a solution of (8S,10M)-26-chloro-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (25.2 mg, 52 μmol) and 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid (30.6 mg, 67.6 μmol) in dimethylformamide (1 mL) was added N-ethyldiisopropylamine (36.2 μL, 208 μmol) and HATU (39.6 mg, 104 μmol) at room temperature. The mixture was stirred at room temperature overnight. The mixture was diluted with EtOAc, washed with saturated aqueous NH₄Cl, washed with brine (2×), dried over anhydrous Na₂SO₄, and concentrated in vacuo. The crude residue was purified by reverse-phase chromatography (40-100% MeCN in water with 0.1% FA) to provide (8S,10M)-26-chloro-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (24.5 mg, 27.5 μmol) as a white solid. m/z (ESI-pos) M+1=882.5; ¹H NMR (400 MHz, CHLOROFORM-D) δ 11.37-11.19 (m, 1H), 7.74-7.66 (m, 1H), 7.62-7.55 (m, 1H), 7.54-7.31 (m, 4H), 7.22-7.02 (m, 4H), 6.78-6.65 (m, 1H), 6.57-5.89 (m, 2H), 5.87-5.21 (m, 1H), 4.97-4.43 (m, 3H), 4.42-4.24 (m, 1H), 4.22-4.03 (m, 3H), 3.91 (td, J=11.4, 2.6 Hz, 1H), 3.69-3.58 (m, 1H), 3.55 (d, J=11.3 Hz, 1H), 3.47-3.31 (m, 1H), 3.22-2.81 (m, 4H), 1.96-1.82 (m, 1H), 1.81-1.24 (m, 5H), 1.24-1.11 (m, 3H).

### Example 219—Synthesis of Compound 219

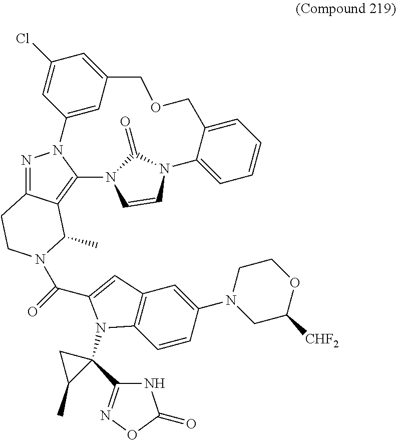

### (8S,10M)-26-chloro-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 219 was synthesized following the procedure of Example 218, with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid being replaced with 5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolecarboxylic acid in step K. m/z (ESI-pos) M+1=864.5; ¹H NMR (400 MHz, CHLOROFORM-D) δ 11.36-11.20 (m, 1H), 7.74-7.66 (m, 1H), 7.61-7.32 (m, 5H), 7.22-7.02 (m, 4H), 6.77-6.65 (m, 1H), 6.57-5.21 (m, 4H), 4.97-4.44 (m, 3H), 4.42-4.24 (m, 1H), 4.16-4.03 (m, 2H), 4.02-3.83 (m, 2H), 3.63 (td, J=13.2, 4.1 Hz, 1H), 3.55-3.46 (m, 1H), 3.46-3.30 (m, 1H), 3.22-2.97 (m, 2H), 2.92 (td, J=11.6, 3.3 Hz, 1H), 2.86-2.75 (m, 1H), 1.95-1.82 (m, 1H), 1.81-1.24 (m, 5H), 1.24-1.09 (m, 3H).

### Example 220—Synthesis of Compound 220

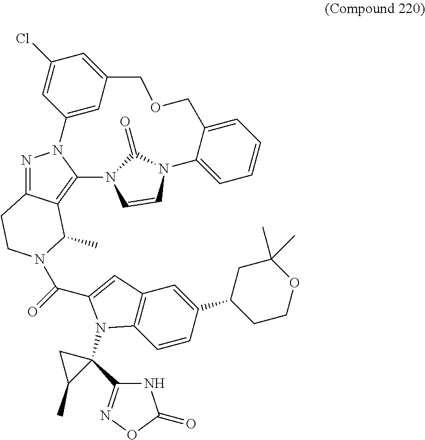

### (8S,10M)-26-chloro-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 220 was synthesized following the procedure of Example 218, with 1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolecarboxylic acid being replaced with 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid in step K. m/z (ESI-pos) M+1=841.5; ¹H NMR (400 MHz, CHLOROFORM-D) δ 11.35-11.20 (m, 1H), 7.75-7.65 (m, 1H), 7.63-7.32 (m, 6H), 7.30-7.26 (m, 1H), 7.23-7.15 (m, 1H), 7.11-7.02 (m, 1H), 6.81-6.68 (m, 1H), 6.55-5.88 (m, 2H), 5.87-5.24 (m, 1H), 4.97-4.43 (m, 3H), 4.42-4.25 (m, 1H), 4.16-4.03 (m, 1H), 3.94-3.77 (m, 2H), 3.71-3.35 (m, 1H), 3.24-2.95 (m, 3H), 1.97-1.83 (m, 1H), 1.82-1.59 (m, 9H), 1.32 (d, 6H), 1.24-1.10 (m, 3H).

### Example 221—Synthesis of Compound 221

### Synthetic Scheme

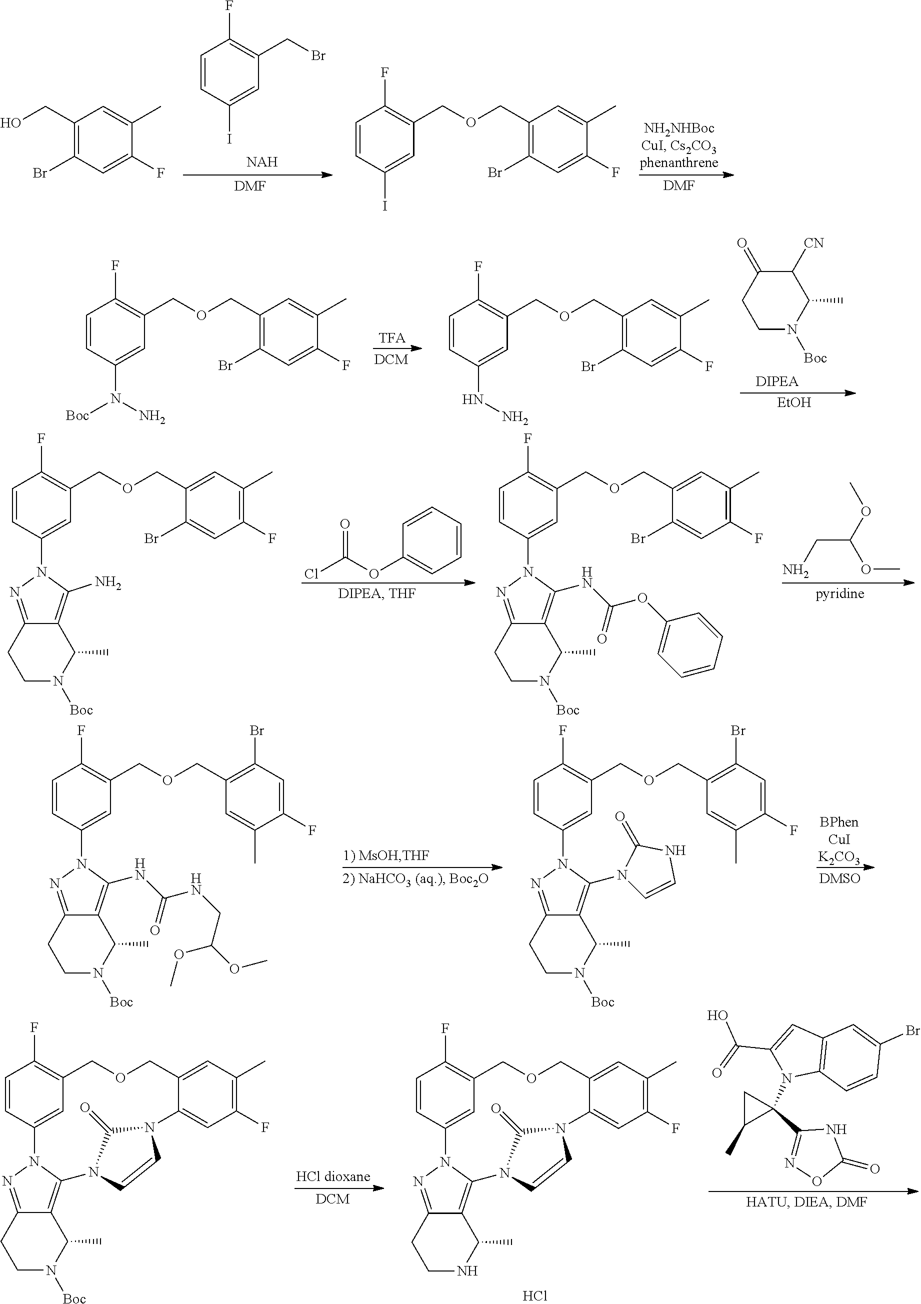

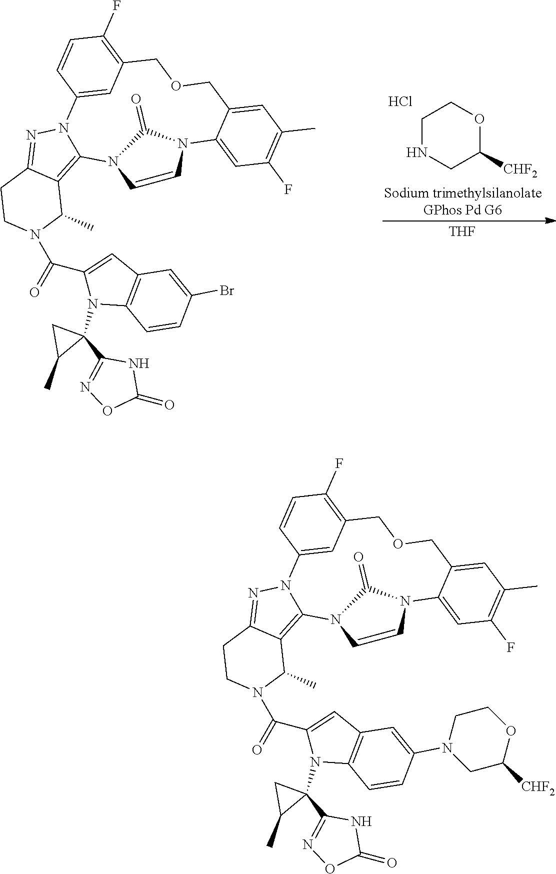

### Procedure for Synthesis of Compound 221

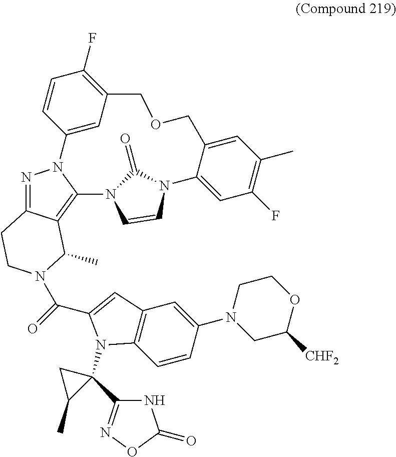

### (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Step A: To a solution of (2-bromo-4-fluoro-5-tolyl)methanol (988 mg, 4.51 mmol) in dimethylformamide (15 mL) was added sodium hydride 60% w/w (271 mg, 6.76 mmol) at 0° C. The reaction was stirred at 0° C. for 20 minutes and 2-(bromomethyl)-1- fluoro-4-iodobenzene (1.63 g, 5.19 mmol) was added portion-wise. The reaction was allowed to warm to room temperature and stirred for 1 hour. The reaction mixture was slowly quenched with saturated aqueous NH₄Cl and extracted with EtOAc. The organics were washed with brine (2×), dried over Na₂SO₄, filtered, and concentrated in vacuo. The crude product was purified by column chromatography (silica gel, 0-15% EtOAc in heptane) to give 4-bromo-2-fluoro-5-{[(2-fluoro-5-iodophenyl)methoxy]methyl}toluene (2.06 g, 4.54 mmol).

Step B: A mixture of 4-bromo-2-fluoro-5-{[(2-fluoro-5-iodophenyl)methoxy]methyl}toluene (2.06 g, 4.54 mmol), tert-butyl carbazate (721 mg, 5.45 mmol), cesium carbonate (2.07 g, 6.36 mmol), copper iodide (86.5 mg, 454 μmol), and 1,10-phenanthroline (328 mg, 1.82 mmol) in DMF (6 mL) was sparged with N₂ for 5 minutes. The reaction mixture was stirred at 80° C. overnight. The reaction mixture was allowed to cool to room temperature, diluted with EtOAc, and filtered through a pad of celite. The filtrate was washed with brine (2×). The organic layer was dried over Na₂SO₄ and concentrated in vacuo. The crude residue was purified by column chromatography (silica gel, 10-50% EtOAc in heptane) to afford tert-butyl 3-(3-{[(2-bromo-4-fluoro-5-tolyl)methoxy]methyl}-4-fluorophenyl)carbazate (1 g, 2.19 mmol). m/z (ESI-pos) M+1=401.1, 403.1 (loss of tBu).

Step C: Trifluoroacetic acid (1.67 mL, 21.9 mmol) was added to a stirred solution of tert-butyl 3-(3-{[(2-bromo-4-fluoro-5-tolyl)methoxy]methyl}-4-fluorophenyl)carbazate (1 g, 2.19 mmol) in dichloromethane (7.3 mL) at room temperature. The reaction was stirred at room temperature for 3 hours. The reaction was quenched with sat. aq. Na₂CO₃, extracted with DCM, washed with brine, dried over Na₂SO₄, filtered, and concentrated in vacuo to provide (3-{[(2-bromo-4-fluoro-5-tolyl)methoxy]methyl}-4-fluorophenyl)hydrazine (781 mg, 2.19 mmol). m/z (ESI-pos) M+1=357.1, 359.1.

Step D: Tert-butyl(2S)-3-cyano-2-methyl-4-oxo-1-piperidinecarboxylate (625 mg, 2.62 mmol) and N-ethyldiisopropylamine (381 μL, 2.19 mmol) were added to a stirred solution of (3-{[(2-bromo-4-fluoro-5-tolyl)methoxy]methyl}-4-fluorophenyl)hydrazine (781 mg, 2.19 mmol) in ethanol (7.3 mL) at room temperature. The mixture was stirred at 80° C. overnight. The reaction mixture was allowed to cool to room temperature, concentrated in vacuo, diluted with water, and extracted with DCM (2×). The organic layers were dried over anhydrous Na₂SO₄ and concentrated in vacuo. The crude compound was purified by column chromatography (silica, 10-50% EtOAc/heptane) to afford tert-butyl (S)-3-amino-2-(3-{[(2-bromo-4-fluoro-5-tolyl)methoxy]methyl}-4-fluorophenyl)-4-methyl-1,2,5-triaza-2,4,6,7-tetrahydro-5H-indene-5-carboxylate (750 mg, 1.3 mmol). m/z (ESI-pos) M+1=577.3, 579.3.

Step E: To a stirred solution of tert-butyl (S)-3-amino-2-(3-{[(2-bromo-4-fluoro-5-tolyl)methoxy]methyl}-4-fluorophenyl)-4-methyl-1,2,5-triaza-2,4,6,7-tetrahydro-5H-indene-5-carboxylate (750 mg, 1.3 mmol) in tetrahydrofuran (6.5 mL) was added N-ethyldiisopropylamine (679 μL, 3.9 mmol) followed by benzenyl chloroformate (326 μL, 2.6 mmol). The reaction mixture was stirred at room temperature for 30 minutes. The reaction mixture was concentrated in vacuo to give crude tert-butyl (S)-2-(3-{[(2-bromo-4-fluoro-5-tolyl)methoxy]methyl}-4-fluorophenyl)-4-methyl-3-[phenyl(oxycarbonylamino)]-1,2,5-triaza-2,4,6,7-tetrahydro-5H-indene-5-carboxylate which was used directly in the next step. m/z (ESI-pos) M+1=697.4.

Step F: 2,2-Dimethoxyethylamine (566 μL, 5.2 mmol) was added to a stirred solution of crude tert-butyl (S)-2-(3-{[(2-bromo-4-fluoro-5-tolyl)methoxy]methyl}-4-fluorophenyl)-4-methyl-3-[phenyl(oxycarbonylamino)]-1,2,5-triaza-2,4,6,7-tetrahydro-5H-indene-5-carboxylate in pyridine (6.5 mL). The mixture was stirred at room temperature for 30 minutes and concentrated in vacuo. The crude product was purified by column chromatography (silica, 10-80% EtOAc in heptane) to give tert-butyl (S)-2-(3-{[(2-bromo-4-fluoro-5-tolyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (590 mg, 833 μmol). m/z (ESI-pos) M+1=708.4, 710.4.

Step G: To a solution of tert-butyl (S)-2-(3-{[(2-bromo-4-fluoro-5-tolyl)methoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (590 mg, 833 μmol) in tetrahydrofuran (8.3 mL) was added methanesulfonic acid (270 μL, 4.16 mmol). The mixture was stirred at 55° C. for 1 hour then allowed to cool to ambient temperature. Saturated aqueous NaHCO₃ (8.3 mL) and tert-butyl-tert-butyl(oxycarbonyloxy) formylate (363 mg, 1.67 mmol) were added, and the mixture was stirred at room temperature for 1 hour. The mixture was extracted with DCM, dried over anhydrous Na₂SO₄, and concentrated in vacuo. The crude residue was purified by column chromatography (silica gel, 10-80% EtOAc in heptane) to give tert-butyl (S)-2-(3-{[(2-bromo-4-fluoro-5-tolyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (560 mg, 869 μmol). m/z (ESI-pos) M+1=644.4, 646.4.

Step H: A mixture of tert-butyl (S)-2-(3-{[(2-bromo-4-fluoro-5-tolyl)methoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (560 mg, 869 μmol), bathophenanthroline (289 mg, 869 μmol), and dipotassium carbonate (0.3 g, 2.17 mmol) in dimethyl sulfoxide (29 mL) was sparged with N₂ for 5 minutes. Copper iodide (124 mg, 652 μmol) was added, and the mixture was sparged with N₂ for 10 minutes. The reaction was stirred at 125° C. for 40 hours. The reaction mixture was allowed to cool to room temperature then filtered through a pad of Celite with EtOAc. The filtrate was washed with saturated aqueous NH₄Cl and washed with brine (2×). The organic layer was dried over sodium sulfate and concentrated in vacuo. The crude residue was purified by column chromatography (silica gel, 5-40% EtOAc in heptane) to give tert-butyl (8S,10M)-17,25-difluoro-8,18-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (79.6 mg, 141 μmol). m/z (ESI-pos) M+1=564.4.

Step I: 4M HCl in dioxane (1 mL) was added to a stirred solution of tert-butyl (8S,10M)-17,25-difluoro-8,18-dimethyl-29-oxo-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-temperature. The solution was stirred at room temperature for 1.5 hours and concentrated in vacuo to provide (8S,10M)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (70.6 mg, 141 μmol). m/z (ESI-pos) M+1=464.3.

Step J: To a solution of (8S,10M)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (70.6 mg, 141 μmol) and 5-bromo-1-[(1S,2S)-2-methyl-1-(5-oxo-4,5-dihydro-1,2,4-oxadiazol-3-yl)cyclopropyl]-2-indolecarboxylic acid (58.7 mg, 155 μmol) in dimethylformamide (1 mL) was added N-ethyldiisopropylamine (98.4 μL, 565 μmol) and HATU (75.2 mg, 198 μmol). The mixture was stirred at room temperature overnight. The reaction mixture was diluted with EtOAc, washed with sat. aq. NH₄Cl, and washed with brine (2×). The organic layer was dried over Na₂SO₄, filtered, and concentrated in vacuo. The crude residue was purified by column chromatography (silica gel, 10-100% EtOAc in heptane) to provide (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (80 mg, 97.1 μmol). m/z (ESI-pos) M+1=823.3, 825.3

Step K: A mixture of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (40 mg, 48.6 μmol), (R)-2-(difluoromethyl)morpholine-hydrogen chloride (1/1) (12.6 mg, 72.8 μmol), bromidopalladium(1+) 2-(trimethylsilyl)ethyl 1-benzenid-4-carboxylate-(3-tert-butoxy-2′,6′-diisopropyl-6-methoxy-2-biphenylyl)dicyclohexylphosphine (1/1) (4.59 mg, 4.86 μmol), and sodium trimethylsilanolate (21.8 mg, 194 μmol) in tetrahydrofuran (1.2 mL) was sparged with N₂ for 2 minutes. The reaction was stirred at 70° C. overnight. The reaction was allowed to cool to room temperature, filtered through Celite with EtOAc, and the filtrate was concentrated. The crude residue was purified by reverse-phase chromatography (40-100% MeCN in water, 0.1% FA) to provide (8S,10M)-7-({5-[(R)-2-(difluoromethyl)-4-morpholinyl]-1-[(1S,2S)-2-methyl-1-(5-oxo-4,5-dihydro-1,2,4-oxadiazol-3-yl)cyclopropyl]-2-indolyl}carbonyl)-17,25-difluoro-8,18-dimethyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (16.5 mg, 18.2 μmol). m/z (ESI-pos) M+1=880.6; ¹H NMR (400 MHz, CHLOROFORM-D) δ 11.31 (s, 1H), 7.74-7.50 (m, 2H), 7.44-7.35 (m, 1H), 7.32 (dd, J=6.1, 2.8 Hz, 1H), 7.24-7.02 (m, 4H), 6.77-6.63 (m, 1H), 6.43-5.21 (m, 4H), 4.97-4.60 (m, 2H), 4.48 (dd, J=13.8, 5.1 Hz, 1H), 4.28-3.81 (m, 5H), 3.64 (td, J=13.1, 4.0 Hz, 1H), 3.54-3.31 (m, 2H), 3.21-2.97 (m, 2H), 2.92 (td, J=11.6, 3.3 Hz, 1H), 2.87-2.76 (m, 1H), 2.39-2.26 (m, 3H), 1.96-1.24 (m, 6H), 1.24-1.08 (m, 3H).

### Example 222—Synthesis of Compound 222

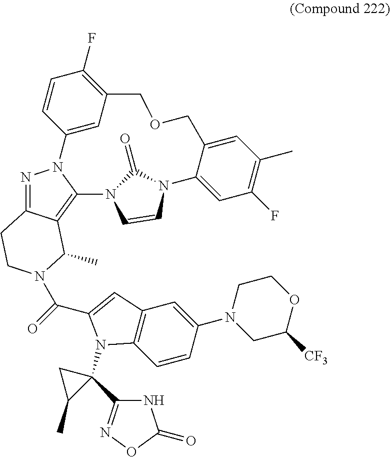

### (8S,10M)-17,25-difluoro-8,18-dimethyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 222 was synthesized following the procedure of Example 221, with (R)-2-(difluoromethyl)morpholine-hydrogen chloride (1/1) being replaced with (R)-2-(trifluoromethyl)morpholine-hydrogen chloride (1/1) in step K. m/z (ESI-pos) M+1=898.6; ¹H NMR (400 MHz, CHLOROFORM-D) δ 11.42-11.16 (m, 1H), 7.66-7.53 (m, 2H), 7.43-7.35 (m, 1H), 7.32 (dd, J=6.1, 2.8 Hz, 1H), 7.24-7.02 (m, 4H), 6.79-6.63 (m, 1H), 6.44-5.85 (m, 2H), 5.85-5.21 (m, 1H), 4.96-4.41 (m, 3H), 4.27-4.05 (m, 4H), 3.91 (td, J=11.4, 2.7 Hz, 1H), 3.64 (td, J=13.2, 4.1 Hz, 1H), 3.55 (d, J=11.4 Hz, 1H), 3.48-3.32 (m, 1H), 3.22-2.81 (m, 4H), 2.38-2.27 (m, 3H), 1.96-1.83 (m, 1H), 1.82-1.24 (m, 5H), 1.24-1.11 (m, 3H).

### Example 223—Synthesis of Compound 223

### Synthetic Scheme

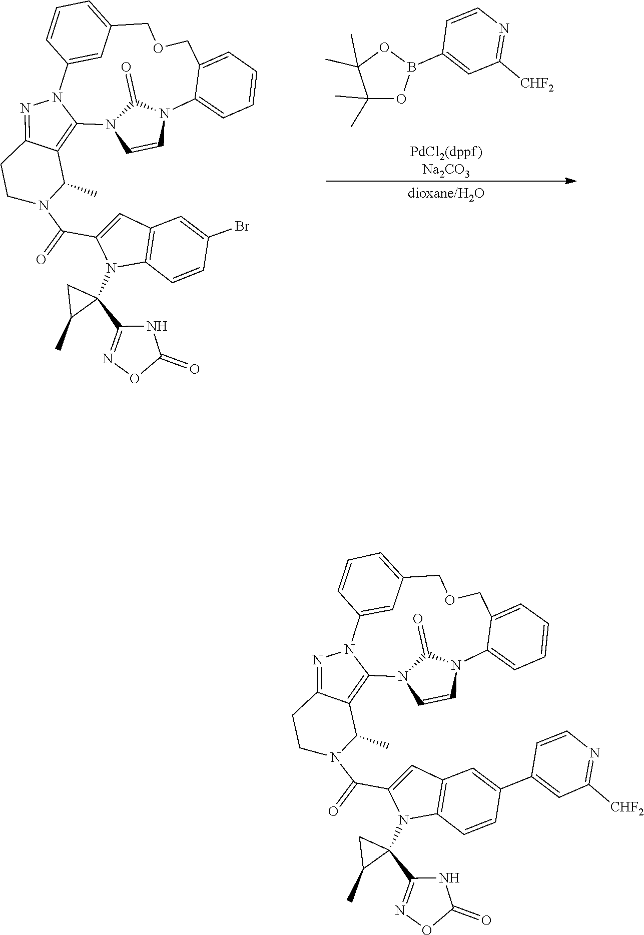

### Procedure for Synthesis of Compound 223

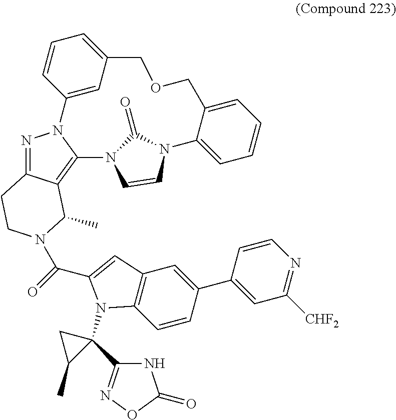

### (8S,10M)-7-({5-[2-(difluoromethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: A solution of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (37.6 mg, 48.6 μmol), PdCl₂(dppf) (19.8 mg, 24.3 μmol), 2-[2-(difluoromethyl)-4-pyridyl]-4,4,5,5-tetramethyl-1,3,2-dioxaborolane (52.5 mg, 206 μmol) and disodium carbonate (25.8 mg, 243 μmol) in 1,4-dioxane (1.22 mL) and water (486 μL) was stirred at 100° C. for 1 hour. The reaction was allowed to cool to room temperature, filtered through Celite with EtOAc, and the filtrate was concentrated. The crude residue was purified by reverse-phase chromatography (40-100% MeCN in water, 0.1% FA) to provide (8S,10M)-7-({5-[2-(difluoromethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (17.8 mg, 21 μmol). m/z (ESI-pos) M+1=822.5; ¹H NMR (400 MHz, CHLOROFORM-D) δ 11.39-11.19 (m, 1H), 8.70 (d, 1H), 8.07-7.98 (m, 1H), 7.95-7.85 (m, 1H), 7.84-7.74 (m, 1H), 7.73-7.58 (m, 3H), 7.56-7.41 (m, 5H), 7.28-7.17 (m, 2H), 7.03-6.54 (m, 2H), 6.38 (dd, J=58.7, 3.1 Hz, 2H), 5.88-5.24 (m, 1H), 5.02-4.04 (m, 5H), 3.79-3.34 (m, 1H), 3.28-2.94 (m, 2H), 2.05-1.11 (m, 9H).

### Example 224—Synthesis of Compound 224

### Synthetic Scheme

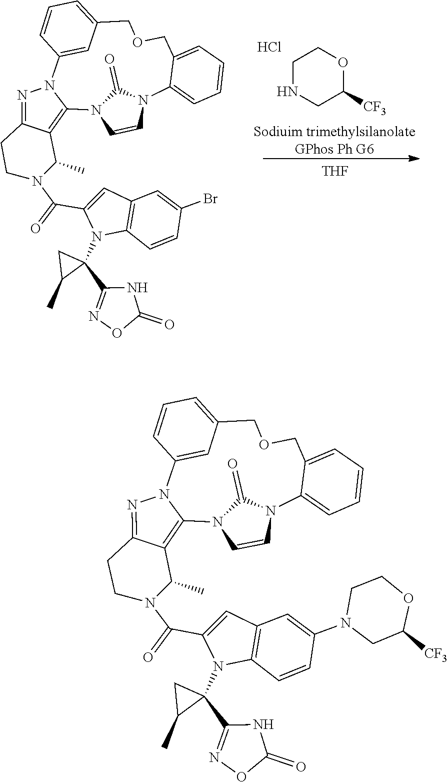

### Procedure for Synthesis of Compound 224

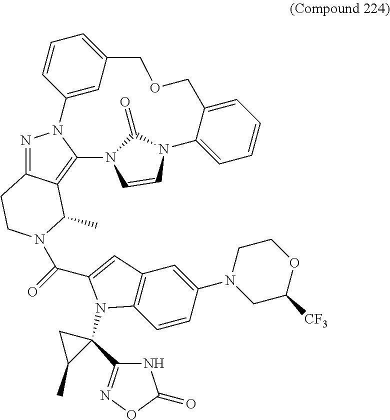

### (8S,10M)-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Step A: A solution of (8S,10M)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (20 mg, 25.9 μmol), [(R)-2-(trifluoromethyl)-4λ⁴-4-morpholinium-4-yl]-λ²-chloranide (7.39 mg, 38.8 μmol), bromidopalladium(1+) 2-(trimethylsilyl)ethyl 1-benzenid-4-carboxylate-(3-tert-butoxy-2′,6′-diisopropyl-6-methoxy-2-biphenylyl)dicyclohexylphosphine (1/1) (2.44 mg, 2.59 μmol), and sodium trimethylsilanolate (11.6 mg, 103 μmol) in tetrahydrofuran (646 μL) was sparged with N₂ for 5 minutes. The reaction was stirred at 70° C. overnight. The reaction was allowed to cool to room temperature, filtered through Celite with EtOAc, and the filtrate was concentrated. The crude residue was purified by reverse-phase chromatography (40-100% MeCN in water, 0.1% FA) to provide (8S,10M)-8-methyl-7-({1-[(1S,2S)-2-methyl-1-(5-oxo-4,5-dihydro-1,2,4-oxadiazol-3-yl)cyclopropyl]-5-[(R)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one (6.67 mg, 7.79 μmol). m/z (ESI-pos) M+1=848.6; ¹H NMR (400 MHz, CHLOROFORM-D) δ 11.39-11.25 (m, 1H), 7.68-7.62 (m, 1H), 7.62-7.55 (m, 1H), 7.55-7.40 (m, 5H), 7.27-7.08 (m, 4H), 6.79-6.63 (m, 1H), 6.48-5.87 (m, 2H), 5.86-5.18 (m, 1H), 4.97-4.55 (m, 2H), 4.48 (dd, J=13.8, 5.0 Hz, 1H), 4.36-4.22 (m, 1H), 4.20-4.07 (m, 3H), 3.91 (td, J=11.4, 2.6 Hz, 1H), 3.65 (td, J=13.1, 4.1 Hz, 1H), 3.55 (d, J=11.5 Hz, 1H), 3.38 (d, J=12.1 Hz, 1H), 3.23-2.82 (m, 4H), 1.95-1.83 (m, 1H), 1.82-1.64 (m, 3H), 1.64-1.48 (m, 2H), 1.26-1.11 (m, 3H).

### Example 225—Synthesis of Compound 225

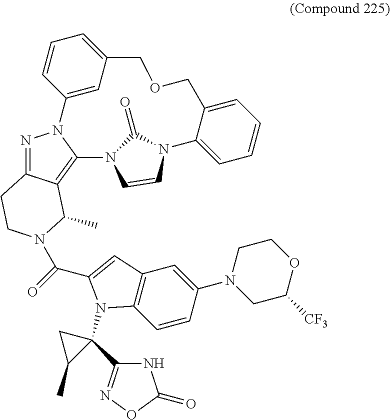

### (8S,10M)-8-methyl-7-({1-[(1S,2S)-2-methyl-1-(5-oxo-4,5-dihydro-1,2,4-oxadiazol-3-yl)cyclopropyl]-5-[(S)-2-(trifluoromethyl)-4-morpholinyl]-2-indolyl}carbonyl)-22-oxa-2,3,7,11,14-pentaazahexacyclo[22.3.1.1¹¹,¹⁴.0²,¹⁰.0⁴,⁹.0¹⁵,²⁰]nonacosa-1(27),3,9,12,15(20),16,18,24(28),25-nonaen-29-one

Compound 225 was synthesized following the procedure of Example 224, with [(R)-2-(trifluoromethyl)-4λ⁴-4-morpholinium-4-yl]-22-chloranide being replaced with [(S)-2-(trifluoromethyl)-4λ⁴-4-morpholinium-4-yl]-22-chloranide in step A. m/z (ESI-pos) M+1=848.6; ¹H NMR (400 MHz, CHLOROFORM-D) δ 11.37-11.24 (m, 1H), 7.68-7.62 (m, 1H), 7.62-7.57 (m, 1H), 7.57-7.41 (m, 5H), 7.28-7.09 (m, 4H), 6.77-6.65 (m, 1H), 6.48-5.85 (m, 2H), 5.85-5.21 (m, 1H), 4.73-4.54 (m, 2H), 4.48 (dd, J=13.6, 5.0 Hz, 1H), 4.36-4.23 (m, 1H), 4.21-4.07 (m, 3H), 3.92 (td, J=11.4, 2.6 Hz, 1H), 3.65 (td, J=13.1, 4.1 Hz, 1H), 3.56 (d, J=11.4 Hz, 1H), 3.38 (d, J=12.0 Hz, 1H), 3.22-2.81 (m, 4H), 1.95-1.83 (m, 1H), 1.82-1.63 (m, 3H), 1.63-1.34 (m, 2H), 1.25-1.09 (m, 3H).

### Example 226—Synthesis of Compound 226

### Synthetic Scheme

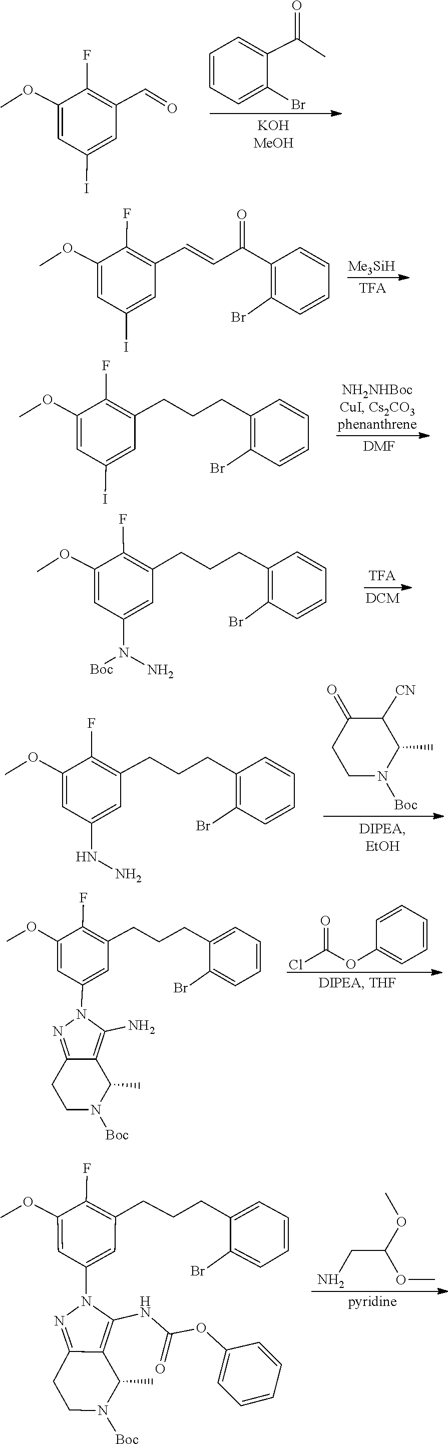

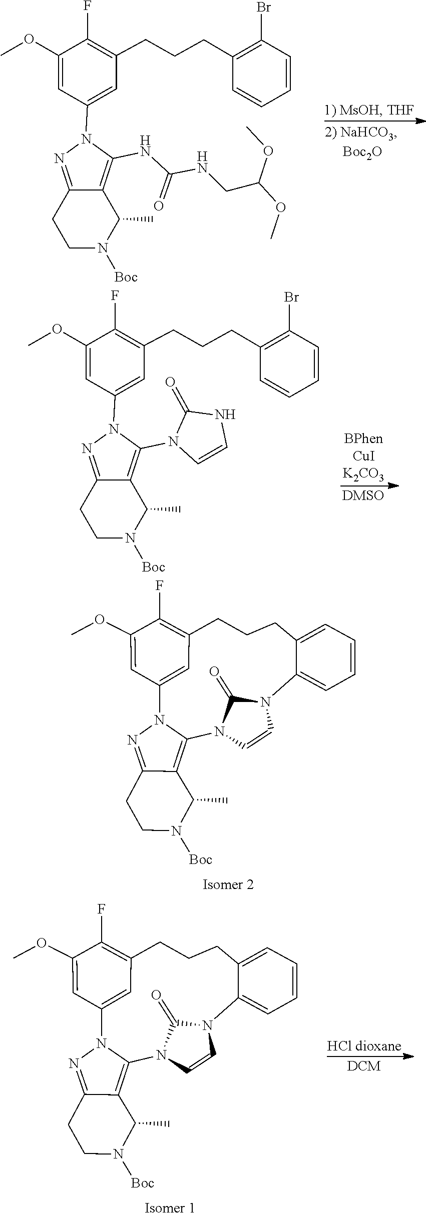

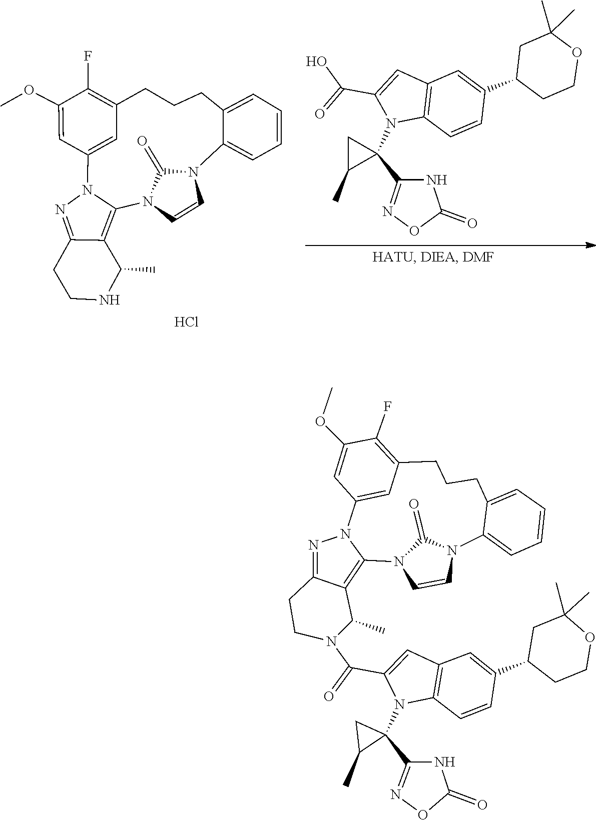

### Procedure for Synthesis of Compound 226

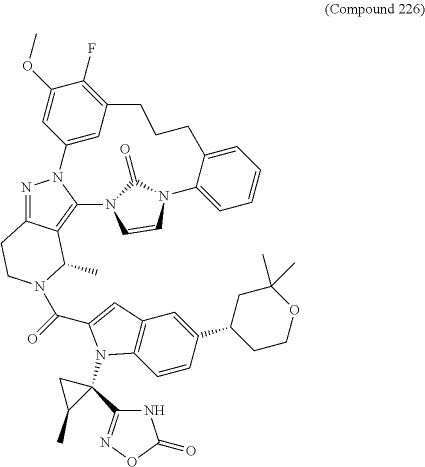

### (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-26-methoxy-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Step A: To a solution of 2-fluoro-5-iodo-3-anisaldehyde (736 mg, 2.63 mmol) and 1-(o-bromophenyl)-1-ethanone (533 mg, 2.68 mmol) in methanol (9 mL) was added potassium hydroxide (442 mg, 7.88 mmol). The reaction was stirred at room temperature overnight. The reaction was diluted with water and extracted with DCM (2×). The organic layers were combined, dried over Na₂SO₄, filtered, and concentrated in vacuo. The crude residue was purified by column chromatography (silica gel, 0-10% EtOAc in heptane) to provide (E)-1-(o-bromophenyl)-3-(2-fluoro-5-iodo-3-methoxyphenyl)-2-propen-1-one (806 mg, 1.75 mmol). ¹H NMR (400 MHz, CHLOROFORM-D) δ 7.63 (dt, J=7.8, 1.2 Hz, 1H), 7.53-7.35 (m, 4H), 7.33 (dddd, J=7.9, 7.0, 2.2, 0.9 Hz, 1H), 7.23 (dt, J=7.6, 1.5 Hz, 1H), 7.18-7.09 (m, 1H), 5.28 (d, J=0.7 Hz, 1H), 3.86 (s, 3H).

Step B: To a solution of (E)-1-(o-bromophenyl)-3-(2-fluoro-5-iodo-3-methoxyphenyl)-2-propen-1-one (806 mg, 1.75 mmol) in TFA (5.82 mL) was added trimethyl silane (1.75 mL) at room temperature. The reaction was stirred at room temperature overnight. The reaction was diluted with water and extracted with DCM. The organic layer was dried over Na₂SO₄, filtered, and concentrated in vacuo. The crude residue was dissolved in THF (2 mL) and 6M NaOH (2 mL) was added. The mixture was stirred at room temperature for 1 hour, diluted with water, extracted with DCM, dried over Na₂SO₄, filtered, and concentrated in vacuo. The crude residue was purified by column chromatography (silica gel, 0-10% EtOAc in heptane) to afford 1-[3-(o-bromophenyl) propyl]-2-fluoro-5-iodo-3-methoxybenzene (523 mg, 1.04 mmol). 1H NMR (400 MHz, CHLOROFORM-D) δ 7.52 (dd, J=7.9, 1.1 Hz, 1H), 7.28-7.17 (m, 2H), 7.15-7.00 (m, 3H), 3.85 (s, 3H), 2.81-2.72 (m, 2H), 2.66 (td, J=7.7, 1.6 Hz, 2H), 1.97-1.84 (m, 2H).

Step C: A mixture of 1-[3-(o-bromophenyl) propyl]-2-fluoro-5-iodo-3-methoxybenzene (523 mg, 1.16 mmol), tert-butyl carbazate (185 mg, 1.4 mmol), cesium carbonate (531 mg, 1.63 mmol), copper iodide (22.2 mg, 116 μmol), and 1,10-phenanthroline (83.9 mg, 466 μmol) in DMF (1.5 mL) was sparged with N₂ for 5 minutes. The reaction mixture was stirred at 80° C. overnight. The reaction mixture was allowed to cool to room temperature, diluted with EtOAc, and filtered through a pad of celite. The filtrate was washed with brine (2×). The organic layer was dried over Na₂SO₄ and concentrated in vacuo. The crude residue was purified by column chromatography (silica gel, 10-50% EtOAc in heptane) to afford tert-butyl 3-{3-[3-(o-bromophenyl) propyl]-4-fluoro-5-methoxyphenyl}carbazate (378 mg, 833 μmol). m/z (ESI-pos) M+1=353.1, 355.1 (loss of Boc).

Step D: Trifluoroacetic acid (637 μL, 8.33 mmol) was added to a stirred solution of tert-butyl 2-{3-[3-(o-bromophenyl) propyl]-4-fluoro-5-methoxyphenyl}carbazate (378 mg, 833 μmol) in dichloromethane (50 mL) at room temperature. The reaction was stirred at room temperature for 2.5 hours. The reaction was quenched with sat. aq. Na₂CO₃, extracted with DCM, washed with brine, dried over Na₂SO₄, filtered, and concentrated in vacuo to provide {3-[3-(o-bromophenyl) propyl]-4-fluoro-5-methoxyphenyl}hydrazine (294 mg, 832 μmol). m/z (ESI-pos) M+1=353.1, 355.1.

Step E: Tert-butyl(2S)-3-cyano-2-methyl-4-oxo-1-piperidinecarboxylate (238 mg, 999 μmol) and N-ethyldiisopropylamine (217 μL, 1.25 mmol) were added to a stirred solution of {3-[3-(o-bromophenyl) propyl]-4-fluoro-5-methoxyphenyl}hydrazine (294 mg, 832 μmol) in ethanol (2.5 mL) at room temperature. The mixture was stirred at 80° C. for 20 hours. The reaction mixture was allowed to cool to room temperature, concentrated in vacuo, diluted with water, and extracted with DCM (2×). The organic layers were dried over anhydrous Na₂SO₄ and concentrated in vacuo. The crude compound was purified by column chromatography (silica, 10-50% EtOAc/heptane) to afford tert-butyl (S)-3-amino-2-{3-[3-(0-bromophenyl) propyl]-4-fluoro-5-methoxyphenyl}-4-methyl-1,2,5-triaza-2,4,6,7-tetrahydro-5H-indene-5-carboxylate (122 mg, 212 μmol). m/z (ESI-pos) M+1=573.3, 575.3.

Step F: To a stirred solution of tert-butyl (S)-3-amino-2-{3-[3-(o-bromophenyl) propyl]-4-fluoro-5-methoxyphenyl}-4-methyl-1,2,5-triaza-2,4,6,7-tetrahydro-5H-indene-5-carboxylate (122 mg, 212 μmol) in tetrahydrofuran (1.1 mL) was added N-ethyldiisopropylamine (111 μL, 637 μmol) followed by benzenyl chloroformate (53.2 μL, 424 μmol). The reaction mixture was stirred at room temperature for 30 minutes. The reaction mixture was concentrated in vacuo to give crude tert-butyl (S)-2-{3-[3-(o-bromophenyl) propyl]-4-fluoro-5-methoxyphenyl}-4-methyl-3-[phenyl(oxycarbonylamino)]-1,2,5-triaza-2,4,6,7-tetrahydro-5H-indene-5-carboxylate which was used directly in the next step. m/z (ESI-pos) M+1=693.4, 695.4.

Step G: 2,2-Dimethoxyethylamine (92.1 μL, 845 μmol) was added to a stirred solution of crude tert-butyl (S)-2-{3-[3-(o-bromophenyl) propyl]-4-fluoro-5-methoxyphenyl}-4-methyl-3-[phenyl(oxycarbonylamino)]-1,2,5-triaza-2,4,6,7-tetrahydro-5H-indene-5-carboxylate in pyridine (1.1 mL). The mixture was stirred at room temperature for 50 minutes and concentrated in vacuo. The crude product was purified by column chromatography (silica, 10-70% EtOAc in heptane) to give tert-butyl (S)-2-{3-[3-(o-bromophenyl) propyl]-4-fluoro-5-methoxyphenyl}-3-[3-(2,2-dimethoxyethyl)ureido]-4-methyl-1,2,5-triaza-2,4,6,7-tetrahydro-5H-indene-5-carboxylate (103 mg, 146 μmol). m/z (ESI-pos) M+1=704.4, 706.4.

Step H: To a solution of tert-butyl (S)-2-{3-[3-(o-bromophenyl) propyl]-4-fluoro-5-methoxyphenyl}-3-[3-(2,2-dimethoxyethyl)ureido]-4-methyl-1,2,5-triaza-2,4,6,7-tetrahydro-5H-indene-5-carboxylate (103 mg, 146 μmol) in tetrahydrofuran (1.5 mL) was added methanesulfonic acid (47.4 μL, 729 μmol). The mixture was stirred at 55° C. for 30 minutes then allowed to cool to ambient temperature. Saturated aqueous NaHCO₃ (1.5 mL) and tert-butyl-tert-butyl(oxycarbonyloxy) formylate (63.7 mg, 292 μmol) were added, and the mixture was stirred at room temperature for 30 minutes. The mixture was extracted with DCM, dried over anhydrous Na₂SO₄, and concentrated in vacuo. The crude residue was purified by column chromatography (silica gel, 10-80% EtOAc in heptane) to give tert-butyl (S)-2-{3-[3-(o-bromophenyl) propyl]-4-fluoro-5-methoxyphenyl}-4-methyl-3-(2-oxo-2,3-dihydro-1H-imidazol-1-yl)-1,2,5-triaza-2,4,6,7-tetrahydro-5H-indene-5-carboxylate (91.2 mg, 142 μmol). m/z (ESI-pos) M+1=640.4, 642.4.

Step I: A mixture of tert-butyl (S)-2-{3-[3-(o-bromophenyl) propyl]-4-fluoro-5-methoxyphenyl}-4-methyl-3-(2-oxo-2,3-dihydro-1H-imidazol-1-yl)-1,2,5-triaza-2,4,6,7-tetrahydro-5H-indene-5-carboxylate (91.2 mg, 142 μmol), bathophenanthroline (47.3 mg, 142 μmol), and dipotassium carbonate (49.2 mg, 356 μmol) in dimethyl sulfoxide (5 mL) was sparged with N₂ for 5 minutes. Copper iodide (20.3 mg, 107 μmol) was added, and the mixture was sparged with N₂ for 10 minutes. The reaction was stirred at 130° C. for 40 hours. The reaction mixture was allowed to cool to room temperature then filtered through a pad of Celite with EtOAc. The filtrate was washed with saturated aqueous NH₄Cl and washed with brine. The organic layer was dried over sodium sulfate and concentrated in vacuo. The crude residue was purified by column chromatography (silica gel, 5-40% EtOAc in heptane) to give tert-butyl (8S,10M)-25-fluoro-26-methoxy-8-methyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (11.6 mg, 20.7 μmol) and tert-butyl (8S,10P)-25-fluoro-26-methoxy-8-methyl-29-oxo-2,3,7,11,14-pentaazahexacyclo[22.3.1.1¹¹,¹⁴.0²,¹⁰.0⁴.⁹.0¹⁵,²⁰]nonacosa-1(28),3,9,12,15,17,19,24,26-nonaene-7-carboxylate (4.9 mg, 8.76 μmol). m/z (ESI-pos) M+1=560.4.

Step J: 4M HCl in dioxane (0.5 mL) was added to a stirred solution of tert-butyl (8S,10M)-25-fluoro-26-methoxy-8-methyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate (11.6 mg, 20.7 μmol) in dichloromethane (0.5 mL) at room temperature. The solution was stirred at room temperature for 1.5 hours and concentrated in vacuo to provide (8S,10M)-25-fluoro-26-methoxy-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (10.3 mg, 20.8 μmol). m/z (ESI-pos) M+1=460.3.

Step K: To a solution of (8S,10M)-25-fluoro-26-methoxy-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one-hydrogen chloride (1/1) (10.3 mg, 20.8 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (14.9 mg, 36.3 μmol) in dimethylformamide (0.73 mL) was added N-ethyldiisopropylamine (18.1 μL, 104 μmol) and HATU (15.8 mg, 41.5 μmol) at room temperature. The mixture was stirred at room temperature for 3 hours. The mixture was diluted with EtOAc, washed with saturated aqueous NH₄Cl, washed with brine, dried over anhydrous Na₂SO₄, and concentrated in vacuo. The crude residue was purified by reverse-phase chromatography (40-100% MeCN in water with 0.1% FA) to provide (8S,10M)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-26-methoxy-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one (4.39 mg, 5.1 μmol). m/z (ESI-pos) M+1=853.6; ¹H NMR (400 MHz, CHLOROFORM-D) δ 11.30 (s, 1H), 7.63-7.49 (m, 2H), 7.45-7.24 (m, 5H), 7.12-6.81 (m, 2H), 6.81-6.64 (m, 1H), 6.32-5.84 (m, 2H), 5.84-5.20 (m, 1H), 4.98-4.40 (m, 1H), 3.98-3.92 (m, 3H), 3.91-3.74 (m, 2H), 3.72-3.34 (m, 1H), 3.25-2.61 (m, 7H), 2.45-1.07 (m, 21H).

### Example 227—Synthesis of Compound 227

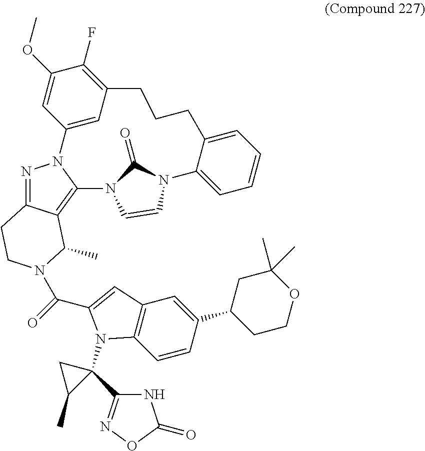

### (8S,10P)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-25-fluoro-26-methoxy-8-methyl-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaen-29-one

Compound 227 was synthesized following the procedure of Example 226, with tert-butyl (8S,10M)-25-fluoro-26-methoxy-8-methyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate being replaced with tert-butyl (8S,10P)-25-fluoro-26-methoxy-8-methyl-29-oxo-2,3,7,11,14-pentaazahexacyclo[22.3.1.1¹¹,¹⁴.0²,¹⁰.0⁴,⁹.0¹⁵,²⁰]nonacosa-1(28),3,9,12,15,17,19,24,26-nonaene-7-carboxylate in step J. m/z (ESI-pos) M+1=853.6; ¹H NMR (400 MHz, CHLOROFORM-D) δ 11.34-11.15 (m, 1H), 7.65-7.49 (m, 2H), 7.48-7.14 (m, 5H), 7.11-6.86 (m, 2H), 6.75-6.00 (m, 3H), 5.92-5.21 (m, 1H), 4.96-4.41 (m, 1H), 3.99-2.59 (m, 13H), 2.42-0.94 (m, 21H).

### Example 228—Synthesis of Compound 228

### Synthetic Scheme

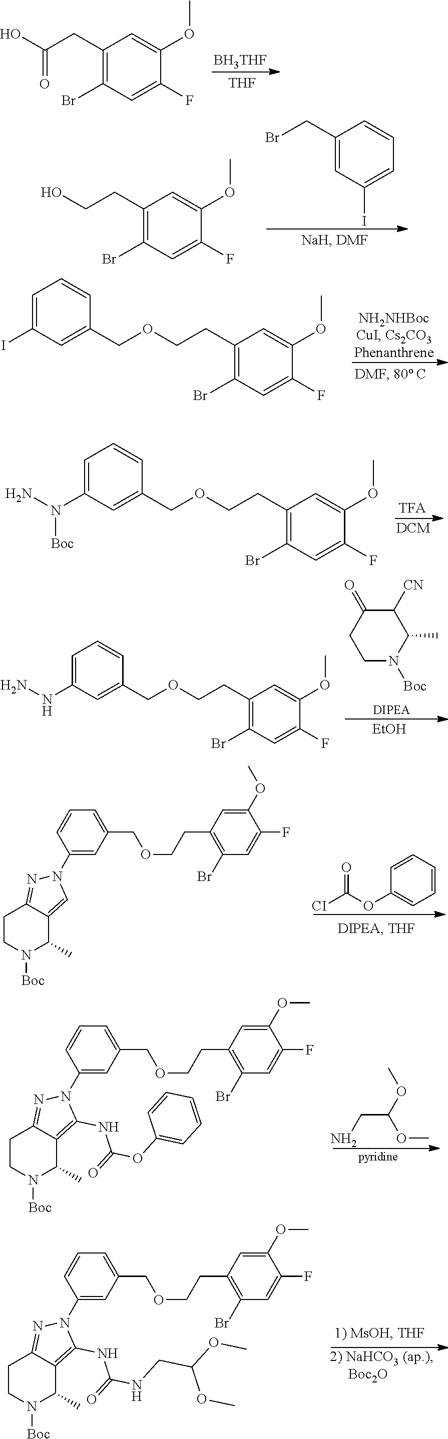

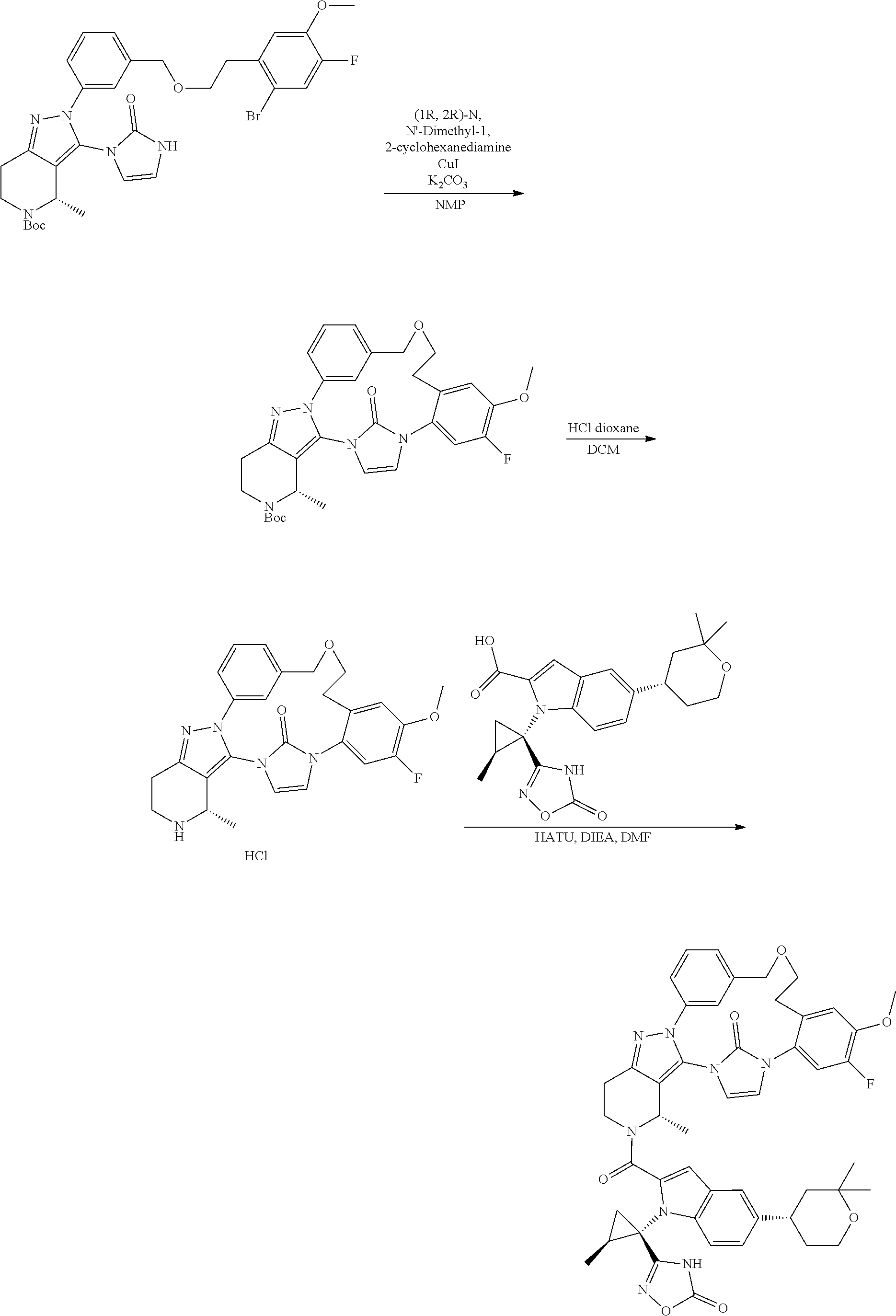

### Procedure for Synthesis of Compound 228

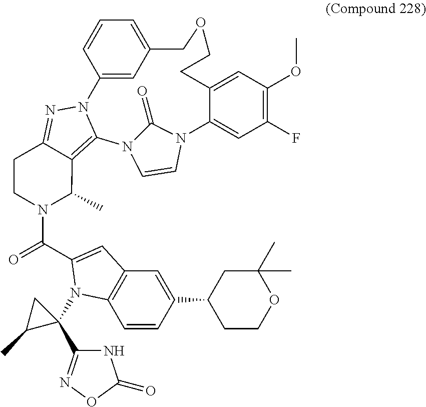

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17-fluoro-18-methoxy-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: 1M Borane in THF (13.6 mL, 13.6 mmol) was added dropwise to a stirred solution of (2-bromo-4-fluoro-5-methoxyphenyl) acetic acid (1.43 g, 5.44 mmol) in tetrahydrofuran (18.1 mL) at 0° C. The mixture was allowed to warm to room temperature and stirred for 30 minutes. The mixture was diluted with sat. aq. NH₄Cl and extracted with ethyl acetate. The organic layer was dried over Na₂SO₄, filtered, and concentrated in vacuo. The crude residue was purified by column chromatography (silica gel, 0-60% EA in heptane) to give 2-(2-bromo-4-fluoro-5-methoxyphenyl) ethanol (1.18 g, 4.75 mmol) as a clear oil. 1H NMR (400 MHz, CHLOROFORM-D) δ 7.15 (dt, J=10.6, 1.8 Hz, 1H), 6.81 (dd, J=8.8, 1.8 Hz, 1H), 3.80-3.75 (m, 3H), 3.72 (q, J=4.9 Hz, 2H), 2.96-2.78 (m, 2H).

Step B: To a solution of 2-(2-bromo-4-fluoro-5-methoxyphenyl) ethanol (1.18 g, 4.75 mmol) and m-(bromomethyl) iodobenzene (2.12 g, 7.13 mmol) in dimethylformamide (9.5 mL) was added NaH 60% w/w (0.2 g, 4.99 mmol) at 0° C. The reaction was stirred at 0° C. for 30 minutes and at room temperature for 30 minutes. The reaction mixture was quenched with saturated aqueous NH₄Cl, diluted with EtOAc, washed with brine (2×). The organic layer was dried over Na₂SO₄ and concentrated in vacuo. The crude product was purified by column chromatography (silica gel, 0-10% EtOAc in heptane) to give 2-bromo-4-fluoro-1-{2-[(m-iodophenyl)methoxy]ethyl}-5-methoxybenzene (1.59 g, 3.43 mmol). 1H NMR (400 MHz, CHLOROFORM-D) δ 7.66-7.56 (m, 2H), 7.29-7.18 (m, 2H), 7.04 (t, J=7.7 Hz, 1H), 6.89 (d, J=8.8 Hz, 1H), 4.44 (s, 2H), 3.84 (d, J=0.9 Hz, 3H), 3.67 (t, J=6.7 Hz, 2H), 3.00 (t, J=6.7 Hz, 2H).

Step C: A mixture of 2-bromo-4-fluoro-1-{2-[(m-iodophenyl)methoxy]ethyl}-5-methoxybenzene (1.6 g, 3.43 mmol), tert-butyl carbazate (544 mg, 4.12 mmol), cesium carbonate (1.57 g, 4.81 mmol), copper iodide (65.4 mg, 343 μmol), and 1,10-phenanthroline (247 mg, 1.37 mmol) in DMF (4.4 mL) was sparged with N₂ for 10 minutes. The reaction mixture was stirred at 80° C. for 21 hours. The reaction mixture was allowed to cool to room temperature, diluted with EtOAc, and filtered through a pad of celite. The filtrate was washed with water (3×). The organic layer was dried over Na₂SO₄ and concentrated in vacuo. The crude residue was purified by column chromatography (silica gel, 10-50% EtOAc in heptane) to afford tert-butyl 2-(m-{[2-(2-bromo-4-fluoro-5-methoxyphenyl)ethoxy]methyl}phenyl)carbazate (1.42 g, 3.02 mmol). m/z (ESI-pos) M+1=413.2, 415.2 (loss of tBu).

Step D: Trifluoroacetic acid (2.31 mL, 30.2 mmol) was added to a stirred solution of tert-butyl 2-(m-{[2-(2-bromo-4-fluoro-5-methoxyphenyl)ethoxy]methyl}phenyl)carbazate (1.42 g, 3.02 mmol) in dichloromethane (6 mL) at room temperature. The reaction was stirred at room temperature for 2 hours. The reaction was quenched with sat. aq. Na₂CO₃, extracted with DCM, washed with brine, dried over Na₂SO₄, filtered, and concentrated in vacuo to provide (m-{[2-(2-bromo-4-fluoro-5-methoxyphenyl)ethoxy]methyl}phenyl)hydrazine (1.11 g, 3.01 mmol). m/z (ESI-pos) M+1=369.2, 371.1.

Step E: Tert-butyl(2S)-3-cyano-2-methyl-4-oxo-1-piperidinecarboxylate (852 mg, 3.57 mmol) and N-ethyldiisopropylamine (778 μL, 4.47 mmol) were added to a stirred solution of (m-{[2-(2-bromo-4-fluoro-5-methoxyphenyl)ethoxy]methyl}phenyl)hydrazine (1.11 g, 3.01 mmol) in ethanol (10 mL) at room temperature. The mixture was stirred at 80° C. overnight. The reaction mixture was allowed to cool to room temperature, concentrated in vacuo, diluted with water, and extracted with DCM (2×). The organic layers were dried over anhydrous Na₂SO₄ and concentrated in vacuo. The crude compound was purified by column chromatography (silica, 10-50% EtOAc/heptane) to afford tert-butyl (S)-3-amino-2-(m-{[2-(2-bromo-4-fluoro-5-methoxyphenyl)ethoxy]methyl}phenyl)-4-methyl-1,2,5-triaza-2,4,6,7-tetrahydro-5H-indene-5-carboxylate (851 mg, 1.44 mmol). m/z (ESI-pos) M+1=589.3, 591.4.

Step F: To a stirred solution of tert-butyl (S)-3-amino-2-(m-{[2-(2-bromo-4-fluoro-5-methoxyphenyl)ethoxy]methyl}phenyl)-4-methyl-1,2,5-triaza-2,4,6,7-tetrahydro-5H-indene-5-carboxylate (851 mg, 1.44 mmol) in tetrahydrofuran (7.2 mL) was added N-ethyldiisopropylamine (754 μL, 4.33 mmol) followed by benzenyl chloroformate (362 μL, 2.89 mmol). The reaction mixture was stirred at room temperature for 30 minutes. The reaction mixture was concentrated in vacuo to give crude tert-butyl (S)-2-(m-{[2-(2-bromo-4-fluoro-5-methoxyphenyl)ethoxy]methyl}phenyl)-4-methyl-3-[phenyl(oxycarbonylamino)]-1,2,5-triaza-2,4,6,7-tetrahydro-5H-indene-5-carboxylate which was used directly in the next step. m/z (ESI-pos) M+1=709.4, 711.4.

Step G: 2,2-Dimethoxyethylamine (629 μL, 5.77 mmol) was added to a stirred solution of crude tert-butyl (S)-2-(m-{[2-(2-bromo-4-fluoro-5-methoxyphenyl)ethoxy]methyl}phenyl)-4-methyl-3-[phenyl(oxycarbonylamino)]-1,2,5-triaza-2,4,6,7-tetrahydro-5H-indene-5-carboxylate in pyridine (7.2 mL). The mixture was stirred at room temperature for 1 hour and concentrated in vacuo. The crude product was purified by column chromatography (silica, 10-100% EtOAc in heptane) to give tert-butyl (S)-2-(m-{[2-(2-bromo-4-fluoro-5-methoxyphenyl)ethoxy]methyl}phenyl)-3-[3-(2,2-dimethoxyethyl)ureido]-4-methyl-1,2,5-triaza-2,4,6,7-tetrahydro-5H-indene-5-carboxylate (763 mg, 1.06 mmol). m/z (ESI-pos) M+1=720.4, 722.4.

Step H: To a solution of tert-butyl (S)-2-(m-{[2-(2-bromo-4-fluoro-5-methoxyphenyl)ethoxy]methyl}phenyl)-3-[3-(2,2-dimethoxyethyl)ureido]-4-methyl-1,2,5-triaza-2,4,6,7-tetrahydro-5H-indene-5-carboxylate (763 mg, 1.06 mmol) in tetrahydrofuran (11 mL) was added methanesulfonic acid (344 μL, 5.29 mmol). The mixture was stirred at 55° C. for 1 hour then allowed to cool to ambient temperature. Saturated aqueous NaHCO₃ (11 mL) and tert-butyl-tert-butyl(oxycarbonyloxy) formylate (462 mg, 2.12 mmol) were added, and the mixture was stirred at room temperature for 15 minutes. The mixture was extracted with DCM, dried over anhydrous Na₂SO₄, and concentrated in vacuo. The crude residue was purified by column chromatography (silica gel, 10-80% EtOAc in heptane) to give tert-butyl (S)-2-(m-{[2-(2-bromo-4-fluoro-5-methoxyphenyl)ethoxy]methyl}phenyl)-4-methyl-3-(2-oxo-2,3-dihydro-1H-imidazol-1-yl)-1,2,5-triaza-2,4,6,7-tetrahydro-5H-indene-5-carboxylate (606 mg, 922 μmol). m/z (ESI-pos) M+1=656.4, 658.4.

Step I: A mixture of tert-butyl (S)-2-(m-{[2-(2-bromo-4-fluoro-5-methoxyphenyl)ethoxy]methyl}phenyl)-4-methyl-3-(2-oxo-2,3-dihydro-1H-imidazol-1-yl)-1,2,5-triaza-2,4,6,7-tetrahydro-5H-indene-5-carboxylate (201 mg, 306 μmol), dipotassium carbonate (127 mg, 919 μmol), and (1R,2R)-1,2-bis(methylamino)cyclohexane (72.5 μL, 460 μmol) in NMP (9.3 mL) was sparged with N₂ for 5 minutes. Copper iodide (58.4 mg, 306 μmol) was added, and the solution was sparged with N₂ for 5 minutes. The reaction was stirred at 130° C. for 3 hours. The solution was allowed to cool to ambient temperature, diluted with EtOAc, washed with water, washed with brine, dried over Na₂SO₄, filtered, and concentrated in vacuo. The crude product was purified by column chromatography (silica gel, 10-80% EtOAc/heptane) to give tert-butyl (S)-17-fluoro-18-methoxy-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (57 mg, 99 μmol). m/z (ESI-pos) M+1=576.4

Step J: 4M HCl in dioxane (0.5 mL) was added to a stirred solution of tert-butyl (S)-17-fluoro-18-methoxy-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (57 mg, 99 μmol) in dichloromethane (0.5 mL) at room temperature. The solution was stirred at room temperature for 30 minutes and concentrated in vacuo to provide(S)-17-fluoro-18-methoxy-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (50.7 mg, 99 μmol). m/z (ESI-pos) M+1=476.3.

Step K: To a solution of (S)-17-fluoro-18-methoxy-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (50.7 mg, 99 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (53 mg, 129 μmol) in dimethylformamide (1 mL) was added N-ethyldiisopropylamine (86.2 μL, 495 μmol) and HATU (75.3 mg, 198 μmol) at room temperature. The mixture was stirred at room temperature for 16 hours. The mixture was diluted with EtOAc, washed with saturated aqueous NH₄Cl, washed with water, washed with brine, dried over anhydrous Na₂SO₄, and concentrated in vacuo. The crude residue was purified by reverse-phase chromatography (40-100% MeCN in water with 0.1% FA) to provide(S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17-fluoro-18-methoxy-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (42.9 mg, 48.8 μmol) as a white solid. m/z (ESI-pos) M+1=869.6; ¹H NMR (400 MHz, CHLOROFORM-D) δ 11.38-11.19 (m, 1H), 7.64-7.41 (m, 4H), 7.31-7.12 (m, 3H), 7.12-6.78 (m, 2H), 6.78-6.65 (m, 1H), 6.48-6.07 (m, 2H), 5.95-5.19 (m, 1H), 4.95-4.37 (m, 3H), 3.95-3.76 (m, 5H), 3.71-2.57 (m, 8H), 1.95-1.37 (m, 8H), 1.37-0.97 (m, 11H).

### Example 229—Synthesis of Compound 229

### Synthetic Scheme

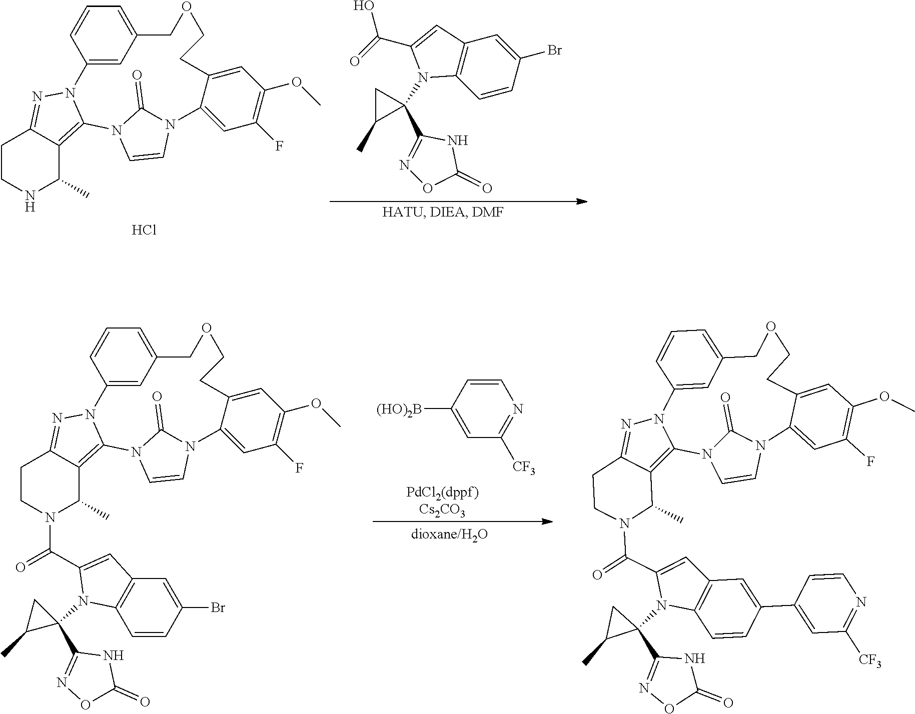

### Procedure for Synthesis of Compound 229

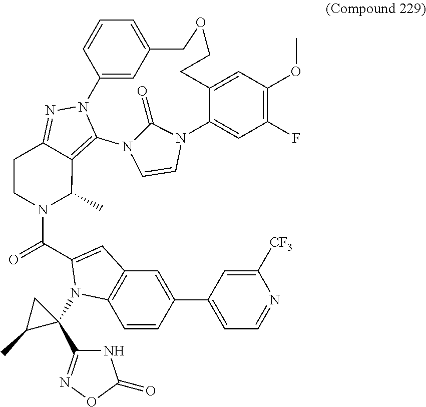

### (S)-17-fluoro-18-methoxy-8-methyl-7-({1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a solution of (S)-17-fluoro-18-methoxy-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (45.5 mg, 88.9 μmol) and 5-bromo-1-[(1S,2S)-2-methyl-1-(5-oxo-4,5-dihydro-1,2,4-oxadiazol-3-yl)cyclopropyl]-2-indolecarboxylic acid (43.7 mg, 116 μmol) in dimethylformamide (1 mL) was added N-ethyldiisopropylamine (77.4 μL, 444 μmol) and HATU (67.8 mg, 178 μmol) at room temperature. The mixture was stirred at room temperature overnight. The mixture was diluted with EtOAc, washed with saturated aqueous NH₄Cl, washed with water, washed with brine, dried over anhydrous Na₂SO₄, and concentrated in vacuo. The crude residue was purified by column chromatography (silica gel, 10-90% EtOAc in heptane) to afford (S)-7-({5-bromo-1-[(1S,2S)-2-methyl-1-(5-oxo-4,5-dihydro-1,2,4-oxadiazol-3-yl)cyclopropyl]-2-indolyl}carbonyl)-17-fluoro-18-methoxy-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (58.2 mg, 69.6 μmol). m/z (ESI-pos) M+1=835.4, 837.

Step B: A solution of (S)-7-({5-bromo-1-[(1S,2S)-2-methyl-1-(5-oxo-4,5-dihydro-1,2,4-oxadiazol-3-yl)cyclopropyl]-2-indolyl}carbonyl)-17-fluoro-18-methoxy-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (20 mg, 23.9 μmol), PdCl₂(dppf) (9.77 mg, 12 μmol), [2-(trifluoromethyl)-4-pyridyl]boranediol (22.8 mg, 120 μmol), and disodium carbonate (12.7 mg, 120 μmol) in 1,4-dioxane (0.6 mL) and water (0.24 mL) was sparged with N₂ for 2 minutes. The reaction was stirred at 80° C. for 1 hour. The reaction was allowed to cool to room temperature, filtered through Celite with EtOAc, and the filtrate was concentrated. The crude residue was purified by reverse-phase chromatography (30-100% MeCN in water, 0.1% FA) to provide(S)-17-fluoro-18-methoxy-8-methyl-7-({1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (10.4 mg, 11.4 μmol) as a peach solid. m/z (ESI-pos) M+1=902.5; ¹H NMR (400 MHz, CHLOROFORM-D) δ 11.40-11.08 (m, 1H), 8.84-8.72 (m, 1H), 8.07-7.86 (m, 2H), 7.84-7.40 (m, 5H), 7.25-7.01 (m, 3H), 6.97-6.74 (m, 2H), 6.49-5.91 (m, 2H), 5.88-5.20 (m, 1H), 5.04-4.37 (m, 3H), 3.95-3.83 (m, 3H), 3.77-3.61 (m, 1H), 3.48-2.99 (m, 5H), 2.85 (dt, J=13.1, 6.7 Hz, 1H), 2.03-1.79 (m, 1H), 1.74-1.43 (m, 5H), 1.28-1.04 (m, 3H).

### Example 230—Synthesis of Compound 230

### Synthetic Scheme

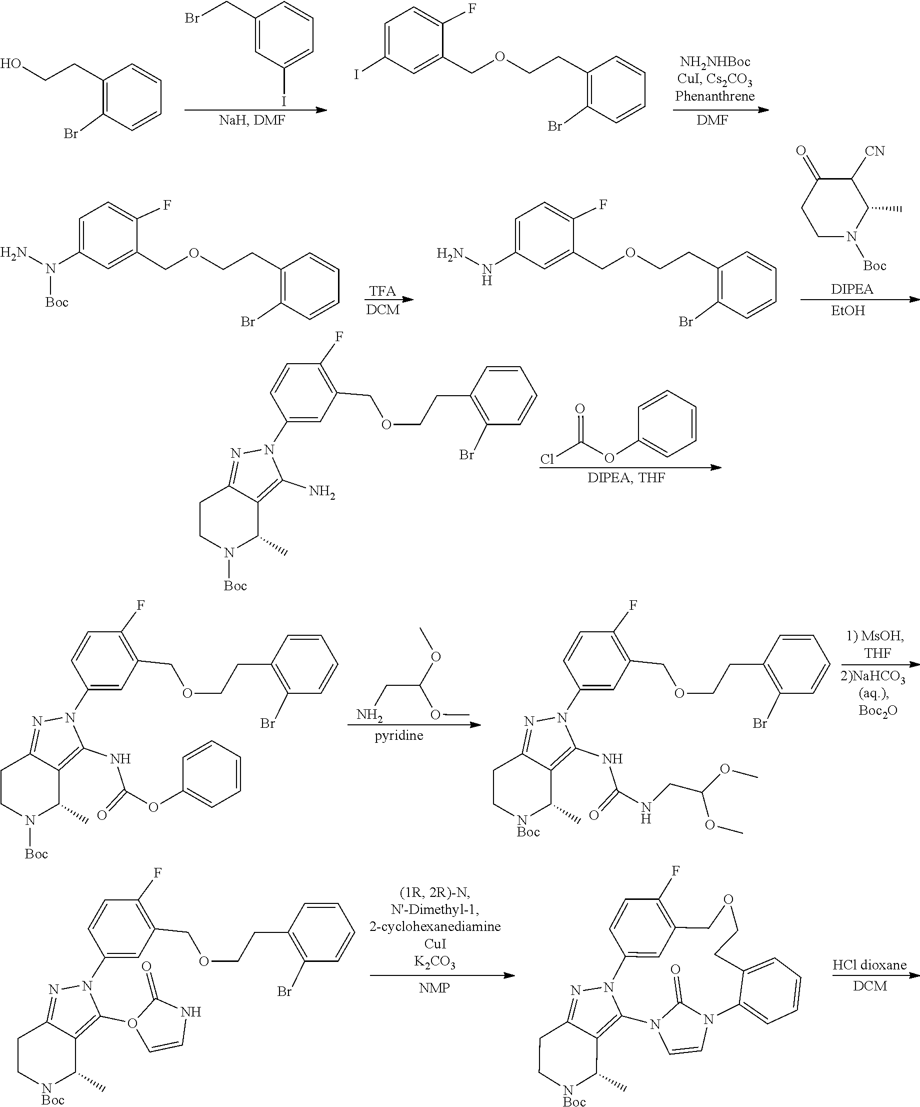

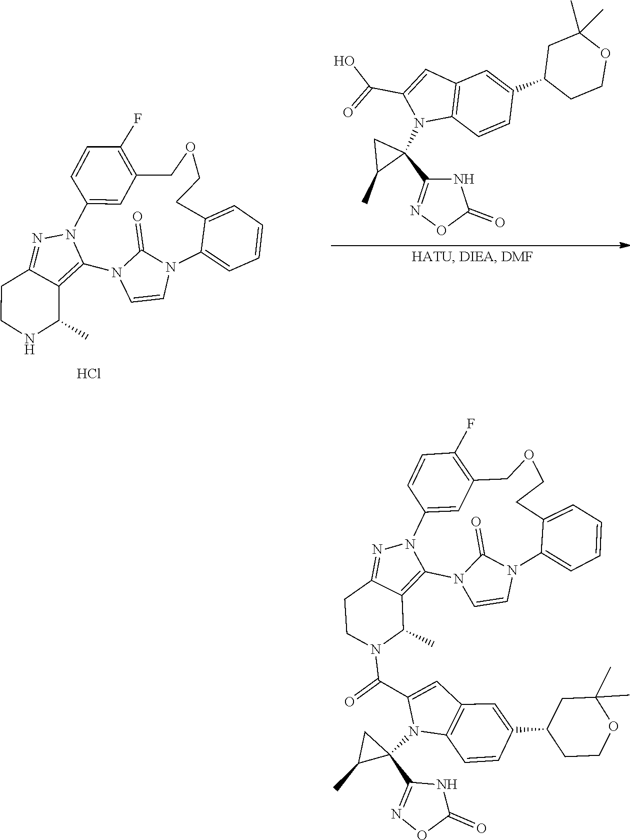

### Procedure for Synthesis of Compound 230

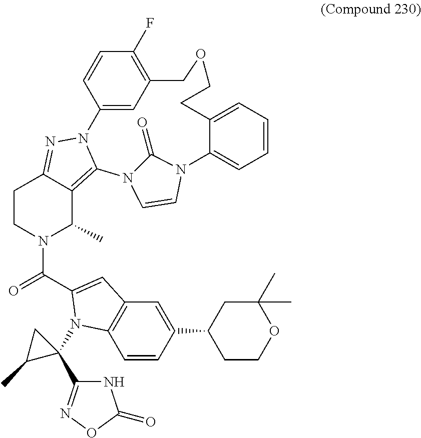

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a solution of 2-(o-bromophenyl) ethanol (1.22 g, 6.05 mmol) and 2-(bromomethyl)-1-fluoro-4-iodobenzene (2.1 g, 6.65 mmol) in dimethylformamide (12 mL) was added NaH 60% w/w (254 mg, 6.35 mmol) at 0° C. The reaction was stirred at 0° C. for 30 minutes and at room temperature for 30 minutes. The reaction mixture was quenched with saturated aqueous NH₄Cl, diluted with ethyl acetate, washed with brine (2×). The organic layer was dried over Na₂SO₄, filtered, and concentrated in vacuo. The crude product was purified by column chromatography (silica gel, 0-10% EtOAc in heptane) to give 2-{[2-(o-bromophenyl)ethoxy]methyl}-1-fluoro-4-iodobenzene (2.3 g, 5.29 mmol).

Step B: A mixture of 2-{[2-(o-bromophenyl)ethoxy]methyl}-1-fluoro-4-iodobenzene (2.3 g, 5.29 mmol), tert-butyl carbazate (838 mg, 6.34 mmol), cesium carbonate (2.41 g, 7.4 mmol), copper iodide (101 mg, 529 μmol), and 1,10-phenanthroline (381 mg, 2.11 mmol) in DMF (6.7 mL) was sparged with N₂ for 5 minutes. The reaction mixture was stirred at 80° C. for 16 hours. The reaction mixture was allowed to cool to room temperature, diluted with EtOAc, and filtered through a pad of celite. The filtrate was washed with brine (2×). The organic layer was dried over Na₂SO₄ and concentrated in vacuo. The crude residue was purified by column chromatography (silica gel, 10-50% EtOAc in heptane) to afford tert-butyl 2-(3-{[2-(o-bromophenyl)ethoxy]methyl}-4-fluorophenyl)carbazate (1.88 g, 4.28 mmol). m/z (ESI-pos) M+1=383.1, 385.1 (loss of tBu).

Step C: Trifluoroacetic acid (6.38 mL, 83.4 mmol) was added to a stirred solution of tert-butyl 2-(3-{[2-(o-bromophenyl)ethoxy]methyl}-4-fluorophenyl)carbazate (1.88 g, 4.28 mmol) in dichloromethane (64 mL) at room temperature. The reaction was stirred at room temperature for 2.25 hours. The reaction was quenched with sat. aq. Na₂CO₃, extracted with DCM, washed with brine, dried over Na₂SO₄, filtered, and concentrated in vacuo to provide (3-{[2-(o-bromophenyl)ethoxy]methyl}-4-fluorophenyl)hydrazine (1.45 g, 4.27 mmol). m/z (ESI-pos) M+1=339.1, 341.1.

Step D: Tert-butyl(2S)-3-cyano-2-methyl-4-oxo-1-piperidinecarboxylate (1.22 g, 5.13 mmol) and N-ethyldiisopropylamine (1.12 mL, 6.41 mmol) were added to a stirred solution of (3-{[2-(o-bromophenyl)ethoxy]methyl}-4-fluorophenyl)hydrazine (1.45 g, 4.27 mmol) in ethanol (14 mL) at room temperature. The mixture was stirred at 80° C. for 16 hours. The reaction mixture was allowed to cool to room temperature, concentrated in vacuo, diluted with water, and extracted with DCM (2×). The organic layers were dried over anhydrous Na₂SO₄ and concentrated in vacuo. The crude compound was purified by column chromatography (silica, 10-50% EtOAc/heptane) to afford tert-butyl (S)-3-amino-2-(3-{[2-(o-bromophenyl)ethoxy]methyl}-4-fluorophenyl)-4-methyl-1,2,5-triaza-2,4,6,7-tetrahydro-5H-indene-5-carboxylate (1.31 g, 2.34 mmol). m/z (ESI-pos) M+1=559.4, 561.3.

Step E: To a stirred solution of tert-butyl (S)-3-amino-2-(3-{[2-(o-bromophenyl)ethoxy]methyl}-4-fluorophenyl)-4-methyl-1,2,5-triaza-2,4,6,7-tetrahydro-5H-indene-5-carboxylate (1.31 g, 2.34 mmol) in tetrahydrofuran (12 mL) was added N-ethyldiisopropylamine (1.22 mL, 7.02 mmol) followed by benzenyl chloroformate (587 μL, 4.68 mmol). The reaction mixture was stirred at room temperature for 30 minutes. The reaction mixture was concentrated in vacuo to give crude tert-butyl (S)-2-(3-{[2-(0-bromophenyl)ethoxy]methyl}-4-fluorophenyl)-4-methyl-3-[phenyl(oxycarbonylamino)]-1,2,5-triaza-2,4,6,7-tetrahydro-5H-indene-5-carboxylate which was used directly in the next step. m/z (ESI-pos) M+1=679.3, 681.4.

Step F: 2,2-Dimethoxyethylamine (1.02 mL, 9.36 mmol) was added to a stirred solution of crude tert-butyl (S)-2-(3-{[2-(o-bromophenyl)ethoxy]methyl}-4-fluorophenyl)-4-methyl-3-[phenyl(oxycarbonylamino)]-1,2,5-triaza-2,4,6,7-tetrahydro-5H-indene-5-carboxylate in pyridine (12 mL). The mixture was stirred at room temperature for 5 minutes and concentrated in vacuo. The crude product was purified by column chromatography (silica, 10-80% EtOAc in heptane) to give tert-butyl (S)-2-(3-{[2-(o-bromophenyl)ethoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (1.09 g, 1.58 mmol). m/z (ESI-pos) M+1=690.4, 692.4.

Step G: To a solution of tert-butyl (S)-2-(3-{[2-(o-bromophenyl)ethoxy]methyl}-4-fluorophenyl)-3-[3-(2,2-dimethoxyethyl)ureido]-4-methyl-1,2,5-triaza-2,4,6,7-tetrahydro-5H-indene-5-carboxylate (1.09 g, 1.58 mmol) in tetrahydrofuran (16 mL) was added methanesulfonic acid (512 μL, 7.89 mmol). The mixture was stirred at 55° C. for 30 minutes then allowed to cool to ambient temperature. Saturated aqueous NaHCO₃ (16 mL) and tert-butyl-tert-butyl(oxycarbonyloxy) formylate (689 mg, 3.16 mmol) were added, and the mixture was stirred at room temperature for 1.5 hours. The mixture was extracted with DCM, dried over anhydrous Na₂SO₄, and concentrated in vacuo. The crude residue was purified by column chromatography (silica gel, 10-80% EtOAc in heptane) to give tert-butyl (S)-2-(3-{[2-(o-bromophenyl)ethoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (940 mg, 1.5 mmol). m/z (ESI-pos) M+1=626.4, 628.4.

Step H: A mixture of tert-butyl (S)-2-(3-{[2-(o-bromophenyl)ethoxy]methyl}-4-fluorophenyl)-4-methyl-3-(2-oxo-2,3-dihydro-1H-imidazol-1-yl)-1,2,5-triaza-2,4,6,7-tetrahydro-5H-indene-5-carboxylate (95 mg, 152 μmol), dipotassium carbonate (62.9 mg, 455 μmol), and (1R,2R)-1,2-bis(methylamino)cyclohexane (35.9 μL, 227 μmol) in NMP (4.6 mL) was sparged with N₂ for 5 minutes. Copper iodide (28.9 mg, 152 μmol) was added, and the mixture was sparged with N₂ for 5 minutes. The reaction was stirred at 100° C. for 16 hours. The mixture was allowed to cool to ambient temperature, diluted with water, and extracted with EtOAc (2×). The combined organic layers were washed with brine, dried over Na₂SO₄, filtered, and concentrated in vacuo. The crude product was purified by column chromatography (silica gel, 10-80% EtOAc/heptane) to give tert-butyl (S)-26-fluoro-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (43.3 mg, 79.4 μmol). m/z (ESI-pos) M+1=546.4.

Step I: 4M HCl in dioxane (1 mL) was added to a stirred solution of tert-butyl (S)-26-fluoro-8-methyl-30-oxo-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (89.2 mg, 163 μmol) in dichloromethane (1 mL) at room temperature. The solution was stirred at room temperature for 1.5 hours and concentrated in vacuo to provide(S)-26-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (78.8 mg, 163 μmol). m/z (ESI-pos) M+1=446.3.

Step J: To a solution of (S)-26-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (78.8 mg, 163 μmol) and 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (87.5 mg, 213 μmol) in dimethylformamide (1.5 mL) was added N-ethyldiisopropylamine (142 μL, 817 μmol) and HATU (124 mg, 327 μmol) at room temperature. The mixture was stirred at room temperature for 2 hours. The mixture was diluted with EtOAc, washed with saturated aqueous NH₄Cl, washed with water, washed with brine, dried over anhydrous Na₂SO₄, and concentrated in vacuo. The crude residue was purified by reverse-phase chromatography (40-100% MeCN in water with 0.1% FA) to provide(S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (96.8 mg, 114 μmol) as a white solid. m/z (ESI-pos) M+1=839.6; ¹H NMR (400 MHz, CHLOROFORM-D) δ 11.32-11.20 (m, 1H), 7.63-7.43 (m, 3H), 7.41-7.09 (m, 7H), 6.79-6.65 (m, 1H), 6.49-6.05 (m, 2H), 5.97-5.19 (m, 1H), 4.95-4.70 (m, 1H), 4.68-4.38 (m, 2H), 3.98-2.73 (m, 10H), 2.02-1.38 (m, 7H), 1.37-1.00 (m, 12H).

### Example 231—Synthesis of Compound 231

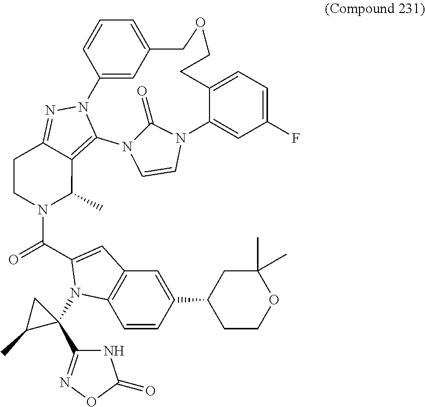 [SMILES: C[C@H]1c2c(nn3c2-n2ccn(c2=O)-c2cc(F)ccc2CCOCc2cccc-3c2)CCN1C(=O)c1cc2cc([C@H]3CCOC(C)(C)C3)ccc2n1[C@@]1(c2noc(=O)[nH]2)C[C@@H]1C]

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 231 was synthesized following the procedure of Example 230, with 2-(0-bromophenyl) ethanol being replaced with 2-(2-bromo-4-fluorophenyl) ethanol and 2-(bromomethyl)-1-fluoro-4-iodobenzene being replaced with m-(bromomethyl) iodobenzene in step A. m/z (ESI-pos) M+1=839.5; ¹H NMR (400 MHz, CHLOROFORM-D) δ 11.34-11.18 (m, 1H), 7.66-7.40 (m, 3H), 7.35-7.13 (m, 5H), 7.13-6.87 (m, 2H), 6.80-6.64 (m, 1H), 6.50-5.93 (m, 2H), 5.93-5.19 (m, 1H), 4.98-4.37 (m, 3H), 3.95-3.74 (m, 2H), 3.72-2.65 (m, 8H), 1.97-1.37 (m, 8H), 1.37-0.92 (m, 11H).

### Example 232—Synthesis of Compound 232

### Synthetic Scheme

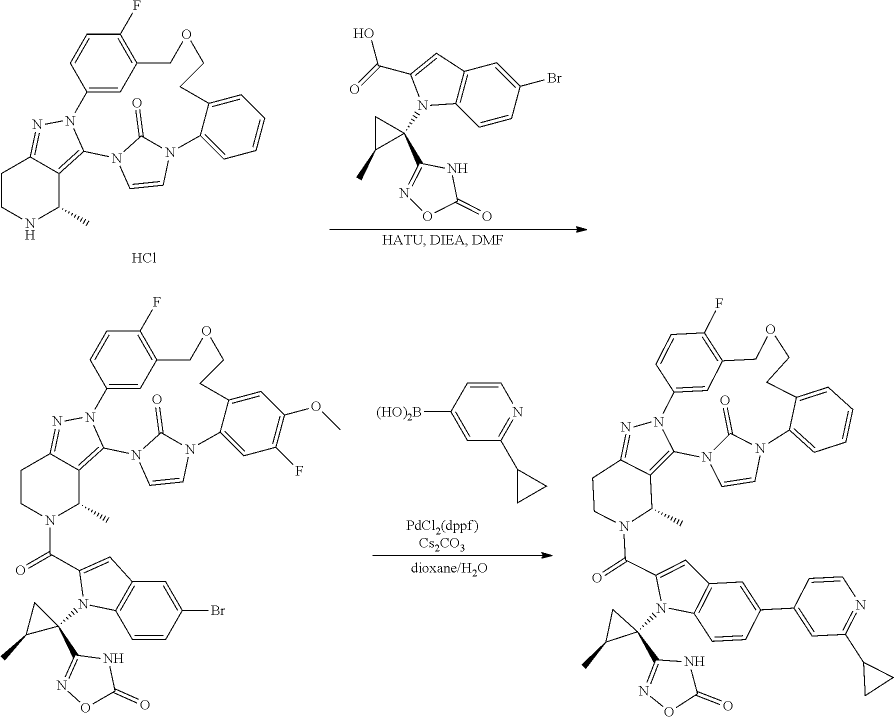

### Procedure for Synthesis of Compound 232

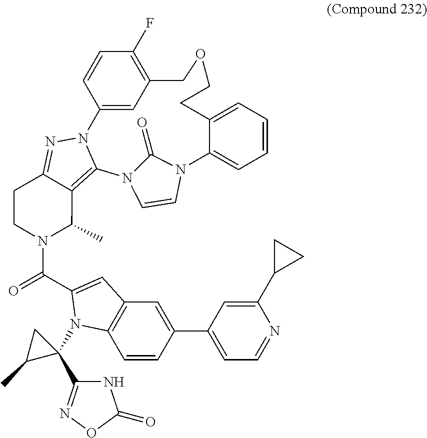 [SMILES: C[C@H]1c2c(nn3c2-n2ccn(c2=O)-c2ccccc2CCOCc2cc-3ccc2F)CCN1C(=O)c1cc2cc(-c3ccnc(C4CC4)c3)ccc2n1[C@@]1(c2noc(=O)[nH]2)C[C@@H]1C]

### (S)-7-{[5-(2-cyclopropyl-4-pyridyl)-1-[(1S,2S)-2-methyl-1-(5-oxo-4,5-dihydro-1,2,4-oxadiazol-3-yl)cyclopropyl]-2-indolyl]carbonyl}-26-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a solution of (S)-26-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (167 mg, 347 μmol) and 5-bromo-1-[(1S,2S)-2-methyl-1-(5-oxo-4,5-dihydro-1,2,4-oxadiazol-3-yl)cyclopropyl]-2-indolecarboxylic acid (144 mg, 381 μmol) in dimethylformamide (1.73 mL) was added N-ethyldiisopropylamine (302 μL, 1.73 mmol) and [(dimethylamino)(3-hydroxy-1λ⁵,2,3,4-tetraaza-3H-inden-1-yl)methylene]bis(methyl)azanium hexafluoridophosphate (1-) (264 mg, 693 μmol). The reaction mixture was stirred at room temperature for 2 hours. The reaction was diluted with EtOAc, washed with sat. aq. NH₄Cl, washed with water, and washed with brine. The organic layer was dried over Na₂SO₄, filtered, and concentrated in vacuo. The crude residue was purified by column chromatography (silica, 10-90% EtOAc in heptane) to afford (S)-7-({5-bromo-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (235 mg, 292 μmol). m/z (ESI-pos) M+1=805.4, 807.4

Step B: A solution of (S)-7-({5-bromo-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (20 mg, 24.8 μmol), PdCl₂(dppf) (10.1 mg, 12.4 μmol), (2-cyclopropyl-4-pyridyl)boranediol (20.2 mg, 124 μmol), and disodium carbonate (13.2 mg, 124 μmol) in 1,4-dioxane (0.62 mL) and water (0.25 mL) was sparged with N₂ for 2 minutes. The reaction was stirred at 100° C. for 1 hour. The reaction was allowed to cool to room temperature, filtered through Celite with EtOAc, and the filtrate was concentrated. The crude residue was purified by reverse-phase chromatography (30-100% MeCN in water, 0.1% FA) to provide(S)-7-{[5-(2-cyclopropyl-4-pyridyl)-1-[(1S,2S)-2-methyl-1-(5-oxo-4,5-dihydro-1,2,4-oxadiazol-3-yl)cyclopropyl]-2-indolyl]carbonyl}-26-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (6.2 mg, 7.27 μmol). m/z (ESI-pos) M+1=844.6; ¹H NMR (400 MHz, CHLOROFORM-D) δ 11.37-11.11 (m, 1H), 8.53-8.42 (m, 1H), 7.99-7.82 (m, 1H), 7.79-7.69 (m, 1H), 7.68-7.50 (m, 3H), 7.41-7.08 (m, 7H), 6.90-6.75 (m, 1H), 6.51-6.01 (m, 2H), 5.98-5.23 (m, 1H), 4.97-4.40 (m, 3H), 3.78-2.76 (m, 7H), 2.20-1.37 (m, 7H), 1.28-0.95 (m, 7H).

### Example 233—Synthesis of Compound 233

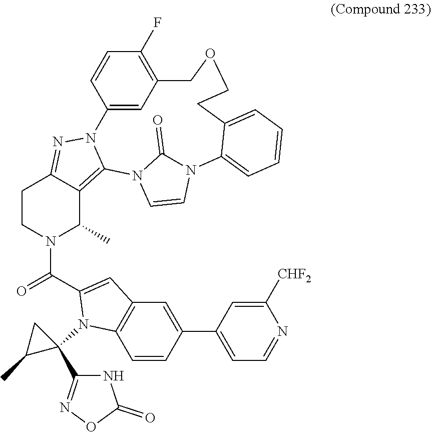 [SMILES: C[C@H]1c2c(nn3c2-n2ccn(c2=O)-c2ccccc2CCOCc2cc-3ccc2F)CCN1C(=O)c1cc2cc(-c3ccnc(C(F)F)c3)ccc2n1[C@@]1(c2noc(=O)[nH]2)C[C@@H]1C]

### (S)-7-({5-[2-(difluoromethyl)-4-pyridyl]-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 233 was synthesized following the procedure of Example 232, with (2-cyclopropyl-4-pyridyl)boranediol being replaced with 2-[2-(difluoromethyl)-4-pyridyl]-4,4,5,5-tetramethyl-1,3,2-dioxaborolane in step B. m/z (ESI-pos) M+1=854.6; ¹H NMR (400 MHz, CHLOROFORM-D) δ 11.34-11.12 (m, 1H), 8.77-8.61 (m, 1H), 8.07-7.95 (m, 1H), 7.93-7.49 (m, 5H), 7.41-7.06 (m, 6H), 6.95-6.53 (m, 2H), 6.52-5.99 (m, 2H), 5.98-5.19 (m, 1H), 5.03-4.29 (m, 3H), 3.81-2.75 (m, 7H), 2.10-0.98 (m, 9H).

### Example 234—Synthesis of Compound 234

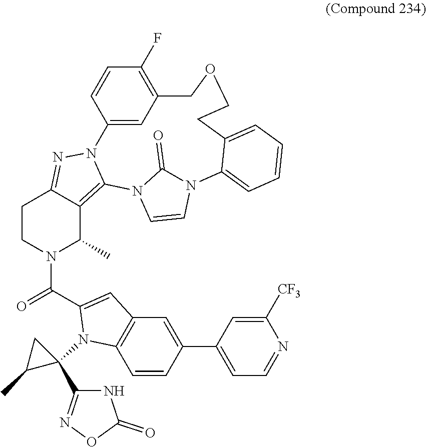 [SMILES: C[C@H]1c2c(nn3c2-n2ccn(c2=O)-c2ccccc2CCOCc2cc-3ccc2F)CCN1C(=O)c1cc2cc(-c3ccnc(C(F)(F)F)c3)ccc2n1[C@@]1(c2noc(=O)[nH]2)C[C@@H]1C]

### (S)-26-fluoro-8-methyl-7-({1-[(1S,2S)-2-methyl-1-(5-oxo-4,5-dihydro-1,2,4-oxadiazol-3-yl)cyclopropyl]-5-[2-(trifluoromethyl)-4-pyridyl]-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 234 was synthesized following the procedure of Example 232, with (2-cyclopropyl-4-pyridyl)boranediol being replaced with [2-(trifluoromethyl)-4-pyridyl]boranediol in step B. m/z (ESI-pos) M+1=872.5; ¹H NMR (400 MHz, CHLOROFORM-D) δ 11.32-11.12 (m, 1H), 8.85-8.68 (m, 1H), 8.08-7.85 (m, 2H), 7.84-7.49 (m, 4H), 7.42-7.06 (m, 6H), 6.95-6.75 (m, 1H), 6.52-6.00 (m, 2H), 5.99-5.16 (m, 1H), 5.00-4.38 (m, 3H), 3.80-2.76 (m, 7H), 2.06-1.37 (m, 6H), 1.28-1.01 (m, 3H).

### Example 235—Synthesis of Compound 235

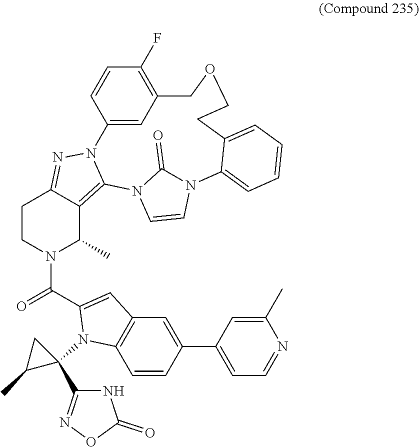

### (S)-26-fluoro-8-methyl-7-({1-[(1S,2S)-2-methyl-1-(5-oxo-4,5-dihydro-1,2,4-oxadiazol-3-yl)cyclopropyl]-5-(2-methyl-4-pyridyl]-2-indolyl}carbonyl)-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 235 was synthesized following the procedure of Example 232, with (2-cyclopropyl-4-pyridyl)boranediol being replaced with (2-methyl-4-pyridyl)boranediol in step B. m/z (ESI-pos) M+1=818.5; ¹H NMR (400 MHz, CHLOROFORM-D) δ 11.26 (s, 1H), 8.55 (d, J=5.2 Hz, 1H), 8.01-7.86 (m, 1H), 7.79-7.62 (m, 2H), 7.61-7.48 (m, 1H), 7.46-7.10 (m, 7H), 6.92-6.76 (m, 1H), 6.51-5.88 (m, 2H), 5.87-5.19 (m, 1H), 5.00-4.38 (m, 3H), 3.79-2.82 (m, 7H), 2.64 (s, 4H), 2.04-1.10 (m, 9H).

### Example 236—Synthesis of Compound 236

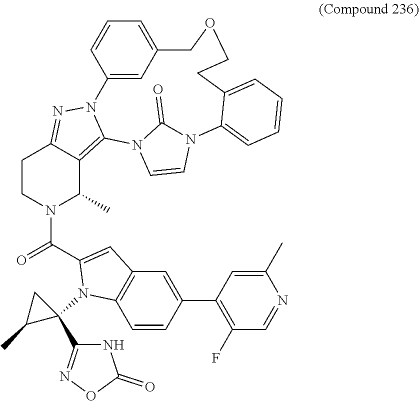

### (S)-7-{[5-(5-fluoro-2-methyl-4-pyridyl)-1-[(1S,2S)-2-methyl-1-(5-oxo-4,5-dihydro-1,2,4-oxadiazol-3-yl)cyclopropyl]-2-indolyl]carbonyl}-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 236 was synthesized following the procedure of Example 232, with (2-cyclopropyl-4-pyridyl)boranediol and(S)-7-({5-bromo-1-[(1S,2S)-2-methyl-1-(5-oxo-4,5-dihydro-1,2,4-oxadiazol-3-yl)cyclopropyl]-2-indolyl}carbonyl)-26-fluoro-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one being replaced with (5-fluoro-2-methyl-4-pyridyl)boranediol and(S)-7-({5-bromo-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one in step B. m/z (ESI-pos) M+1=818.5; ¹H NMR (400 MHz, CHLOROFORM-D) δ 11.32-11.17 (m, 1H), 8.48-8.36 (m, 1H), 7.98-7.86 (m, 1H), 7.80-7.43 (m, 4H), 7.39-7.07 (m, 7H), 6.92-6.72 (m, 1H), 6.56-5.90 (m, 2H), 5.90-5.20 (m, 1H), 5.01-4.38 (m, 3H), 3.80-2.78 (m, 7H), 2.66-2.54 (m, 3H), 2.04-1.01 (m, 9H).

### Example 237—Synthesis of Compound 237

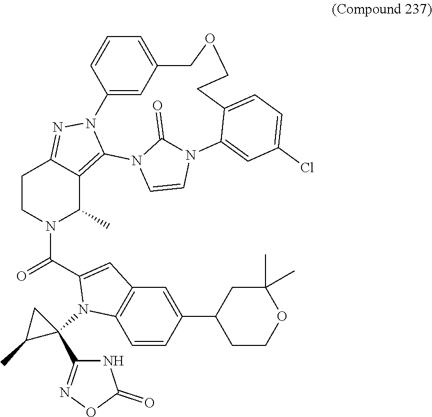 [SMILES: C[C@H]1c2c(nn3c2-n2ccn(c2=O)-c2cc(Cl)ccc2CCOCc2cccc-3c2)CCN1C(=O)c1cc2cc(C3CCOC(C)(C)C3)ccc2n1[C@@]1(c2noc(=O)[nH]2)C[C@@H]1C]

### (S)-17-chloro-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-23-oxa-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 237 was synthesized following the procedure of Example 23, with 2-(o-bromophenyl) ethanol being replaced with 2-(2-bromo-5-chlorophenyl) ethanol in step A. m/z (ESI-pos) M+1=855.5; ¹H NMR (400 MHz, CHLOROFORM-D) δ 11.36-11.19 (m, 1H), 7.65-7.42 (m, 4H), 7.38-7.04 (m, 6H), 6.81-6.64 (m, 1H), 6.50-5.92 (m, 2H), 5.92-5.17 (m, 1H), 4.97-4.30 (m, 3H), 3.96-3.72 (m, 2H), 3.72-2.67 (m, 8H), 1.98-1.47 (m, 7H), 1.47-0.99 (m, 12H).

### Example 238—Synthesis of Compound 238

### Synthetic Scheme

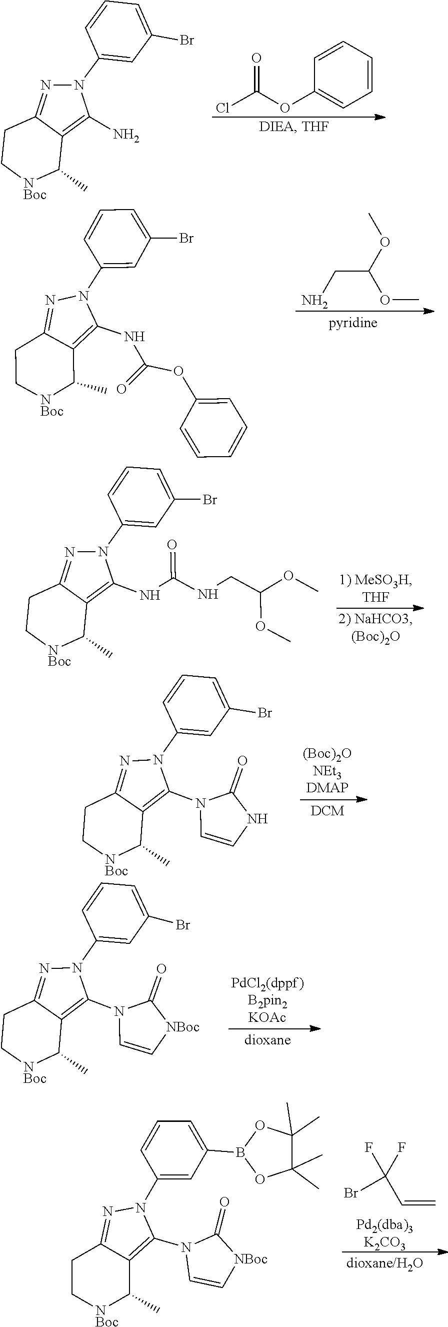

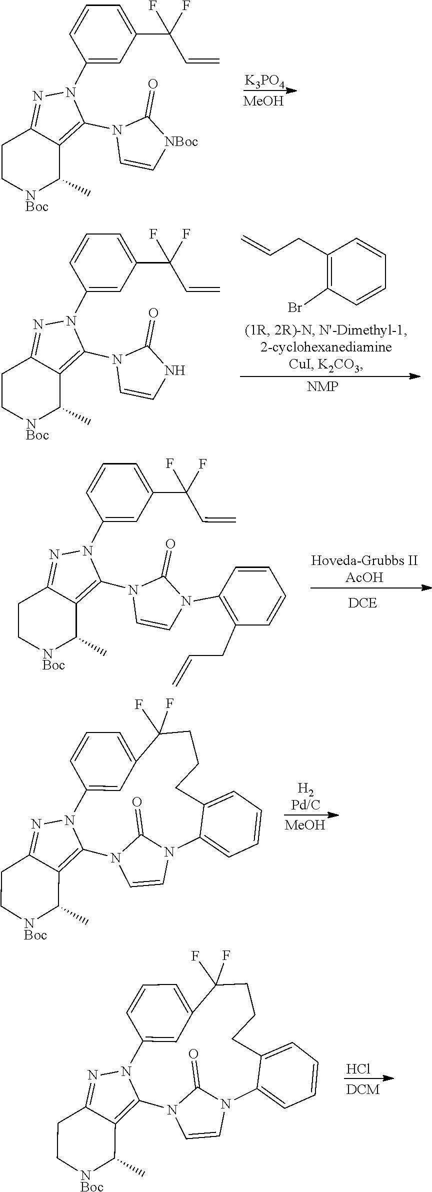

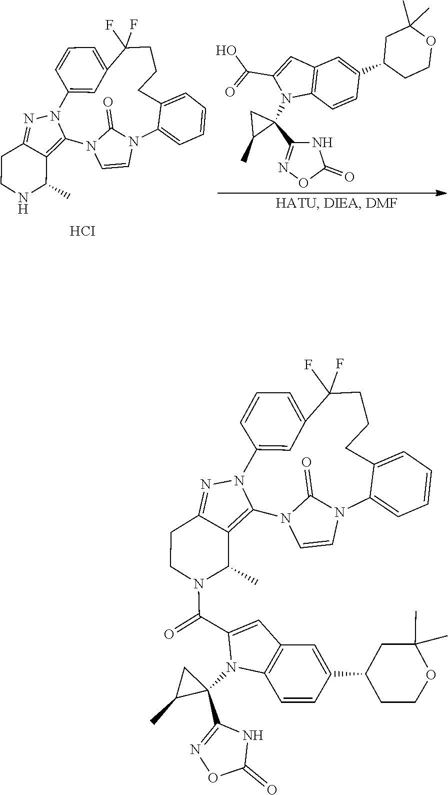

### Procedure for Synthesis of Compound 238

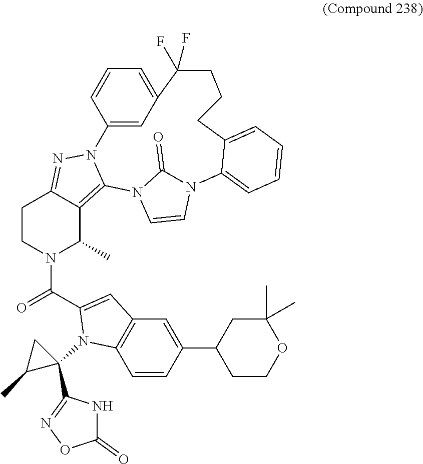

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-24,24-difluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Step A: To a stirred solution of tert-butyl (S)-3-amino-2-(m-bromophenyl)-4-methyl-1,2,5-triaza-2,4,6,7-tetrahydro-5H-indene-5-carboxylate (1.5 g, 3.68 mmol) in tetrahydrofuran (18 mL) was added N-ethyldiisopropylamine (1.92 mL, 11 mmol) followed by benzenyl chloroformate (924 μL, 7.37 mmol). The reaction mixture was stirred at room temperature for 30 minutes. The reaction mixture was concentrated in vacuo to give crude tert-butyl (S)-2-(m-bromophenyl)-4-methyl-3-[phenyl(oxycarbonylamino)]-1,2,5-triaza-2,4,6,7-tetrahydro-5H-indene-5-carboxylate which was used directly in the next step. m/z (ESI-pos) M+1=527.2, 529.2.

Step B: 2,2-Dimethoxyethylamine (802 μL, 7.36 mmol) was added to a stirred solution of crude tert-butyl (S)-2-(m-bromophenyl)-4-methyl-3-[phenyl(oxycarbonylamino)]-1,2,5-triaza-2,4,6,7-tetrahydro-5H-indene-5-carboxylate in pyridine (18 mL). The mixture was stirred at room temperature overnight and concentrated in vacuo. The crude product was purified by column chromatography (silica, 10-80% EtOAc in heptane) to give tert-butyl (S)-2-(m-bromophenyl)-3-[3-(2,2-dimethoxyethyl)ureido]-4-methyl-1,2,5-triaza-2,4,6,7-tetrahydro-5H-indene-5-carboxylate (1.68 g, 3.12 mmol). m/z (ESI-pos) M+1=538.3, 540.3.

Step C: To a solution of tert-butyl (S)-2-(m-bromophenyl)-3-[3-(2,2-dimethoxyethyl)ureido]-4-methyl-1,2,5-triaza-2,4,6,7-tetrahydro-5H-indene-5-carboxylate (1.68 g, 3.12 mmol) in tetrahydrofuran (31 mL) was added methanesulfonic acid (1.01 mL, 15.6 mmol). The mixture was stirred at 55° C. for 1 hour then allowed to cool to ambient temperature. Saturated aqueous NaHCO₃ (31 mL) and tert-butyl-tert-butyl(oxycarbonyloxy) formylate (1.02 g, 4.68 mmol) were added, and the mixture was stirred at room temperature for 1 hour. The mixture was extracted with DCM, dried over anhydrous Na₂SO₄, and concentrated in vacuo. The crude residue was purified by column chromatography (silica gel, 10-80% EtOAc in heptane) to give tert-butyl (S)-2-(m-bromophenyl)-4-methyl-3-(2-oxo-2,3-dihydro-1H-imidazol-1-yl)-1,2,5-triaza-2,4,6,7-tetrahydro-5H-indene-5-carboxylate (1.07 g, 2.25 mmol). m/z (ESI-pos) M+1=474.4, 476.4.

Step D: To solution of tert-butyl (S)-2-(m-bromophenyl)-4-methyl-3-(2-oxo-2,3-dihydro-1H-imidazol-1-yl)-1,2,5-triaza-2,4,6,7-tetrahydro-5H-indene-5-carboxylate (918 mg, 1.94 mmol), N,N-dimethyl-4-pyridylamine (23.6 mg, 194 μmol), and tert-butyl-tert-butyl(oxycarbonyloxy) formylate (845 mg, 3.87 mmol) in dichloromethane (6.5 mL) was added triethylamine (810 μL, 5.81 mmol) at room temperature. The reaction was stirred at room temperature for 1 hour and concentrated in vacuo. The crude product was purified by column chromatography (silica gel, 10-60% EtOAc in heptane) to give tert-butyl (S)-2-(m-bromophenyl)-3-(3-tert-butoxycarbonyl-2-oxo-2,3-dihydro-1H-imidazol-1-yl)-4-methyl-1,2,5-triaza-2,4,6,7-tetrahydro-5H-indene-5-carboxylate (1.09 g, 1.9 mmol). m/z (ESI-pos) M+1=574.3, 576.3.

Step E: A mixture of tert-butyl (S)-2-(m-bromophenyl)-3-(3-tert-butoxycarbonyl-2-oxo-2,3-dihydro-1H-imidazol-1-yl)-4-methyl-1,2,5-triaza-2,4,6,7-tetrahydro-5H-indene-5-carboxylate (0.2 g, 348 μmol), 4,4,5,5-tetramethyl-2-(4,4,5,5-tetramethyl-1,3,2-dioxaborolan-2-yl)-1,3,2-dioxaborolane (177 mg, 696 μmol), potassium acetate (103 mg, 1.04 mmol), and dichloro-palladamethane-iron-1-(diphenylphosphino)-1,2,3,4,5-cyclopentanepentayl (1/1/2) (50.9 mg, 69.6 μmol) in 1,4-dioxane (2.3 mL) was sparged with N₂ for 5 minutes and stirred at 85° C. for 3 hours. The reaction was allowed to cool to room temperature, filtered, and the filtrate was concentrated in vacuo. The crude product was purified by column chromatography (silica gel, 0-80% EtOAc in heptane) to give a tert-butyl (S)-3-(3-tert-butoxycarbonyl-2-oxo-2,3-dihydro-1H-imidazol-1-yl)-4-methyl-2-[m-(4,4,5,5-tetramethyl-1,3,2-dioxaborolan-2-yl)phenyl]-1,2,5-triaza-2,4,6,7-tetrahydro-5H-indene-5-carboxylate (191 mg, 306 μmol). m/z (ESI-pos) M+1=622.5.

Step F: To a vial containing tert-butyl (S)-3-(3-tert-butoxycarbonyl-2-oxo-2,3-dihydro-1H-imidazol-1-yl)-4-methyl-2-[m-(4,4,5,5-tetramethyl-1,3,2-dioxaborolan-2-yl)phenyl]-1,2,5-triaza-2,4,6,7-tetrahydro-5H-indene-5-carboxylate (191 mg, 306 μmol), dipotassium carbonate (127 mg, 919 μmol), and palladium-(1E,4E)-1,5-diphenyl-1,4-pentadien-3-one (2/3) (70.2 mg, 76.6 μmol) was added 1,4-dioxane (1.5 mL) and water (11 μL, 613 μmol) under air. 3-Bromo-3,3-difluoropropene (132 μL, 1.3 mmol) was added and the reaction was stirred at 80° C. for 1 hour. The reaction was allowed to cool to room temperature, filtered, and the filtrate was concentrated in vacuo. The crude product was purified by column chromatography (silica gel, 10-100% EtOAc in heptane) to give tert-butyl (S)-3-(3-tert-butoxycarbonyl-2-oxo-2,3-dihydro-1H-imidazol-1-yl)-2-[m-(1,1-difluoro-2-propenyl)phenyl]-4-methyl-1,2,5-triaza-2,4,6,7-tetrahydro-5H-indene-5-carboxylate (158 mg, 276 μmol). m/z (ESI-pos) M+1=572.4

Step G: A solution of tert-butyl (S)-3-(3-tert-butoxycarbonyl-2-oxo-2,3-dihydro-1H-imidazol-1-yl)-2-[m-(1,1-difluoro-2-propenyl)phenyl]-4-methyl-1,2,5-triaza-2,4,6,7-tetrahydro-5H-indene-5-carboxylate (158 mg, 276 μmol) and tripotassium phosphate (17.6 mg, 82.9 μmol) in methanol (1.4 mL) was stirred at 70° C. for 3 hours. The reaction was allowed to cool to room temperature, filtered, and the filtrate was concentrated in vacuo. The crude product was purified by column chromatography (silica gel, 0-100% EtOAc in heptane) to give tert-butyl (S)-2-[m-(1,1-difluoro-2-propenyl)phenyl]-4-methyl-3-(2-oxo-2,3-dihydro-1H-imidazol-1-yl)-1,2,5-triaza-2,4,6,7-tetrahydro-5H-indene-5-carboxylate (44.1 mg, 93.5 μmol). m/z (ESI-pos) M+1=472.4

Step H: A mixture of tert-butyl (S)-2-[m-(1,1-difluoro-2-propenyl)phenyl]-4-methyl-3-(2-oxo-2,3-dihydro-1H-imidazol-1-yl)-1,2,5-triaza-2,4,6,7-tetrahydro-5H-indene-5-carboxylate (44.1 mg, 93.5 μmol), dipotassium carbonate (38.8 mg, 281 μmol), (1R,2R)-1,2-bis(methylamino)cyclohexane (14.7 μL, 93.5 μmol), o-allylbromobenzene (65.6 mg, 333 μmol), and copper iodide (10.7 mg, 56.1 μmol) in N-methyl-2-pyrrolidone (0.5 mL) was sparged with N₂ for 1 minutes. The reaction was stirred at 130° C. for 2 hours. The reaction was allowed to cool to ambient temperature, diluted with water, and extracted with EtOAc. The organic layer was washed with brine, dried over Na₂SO₄, filtered and concentrated. The crude product was then purified by column chromatography (silica gel, 20-100% EtOAc in heptane) to give tert-butyl (S)-3-[3-(o-allylphenyl)-2-oxo-2,3-dihydro-1H-imidazol-1-yl]-2-[m-(1,1-difluoro-2-propenyl)phenyl]-4-methyl-1,2,5-triaza-2,4,6,7-tetrahydro-5H-indene-5-carboxylate (38.8 mg, 66 μmol). m/z (ESI-pos) M+1=588.4

Step I: Ruthenium o-isopropoxymethanediidyltoluene 1,3-bis(mesityl)-2,2-imidazolidinediide dichloride (31 mg, 49.5 μmol) was added to a solution of tert-butyl (S)-3-[3-(o-allylphenyl)-2-oxo-2,3-dihydro-1H-imidazol-1-yl]-2-[m-(1,1-difluoro-2-propenyl)phenyl]-4-methyl-1,2,5-triaza-2,4,6,7-tetrahydro-5H-indene-5-carboxylate (38.8 mg, 66 μmol) and acetic acid (7.55 μL, 132 μmol) in 1,2-dichloroethane (33 mL, 419 mmol). The mixture was sparged with N₂ for 20 minutes and stirred at 40° C. for 4 hours. Additional ruthenium o-isopropoxymethanediidyltoluene 1,3-bis(mesityl)-2,2-imidazolidinediide dichloride (15 mg) was added and the mixture was sparged with N₂ for 20 minutes. The reaction was stirred at 50° C. for 9 hours. The reaction was allowed to cool to room temperature and washed with saturated aqueous NaHCO₃. The organic layer was dried over sodium sulfate, filtered, and concentrated in vacuo. The crude material was purified by column chromatography (silica gel, 10-60% EtOAc in heptane) to give tert-butyl (8S,22Z)-24,24-difluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (14.2 mg, 25.4 μmol). m/z (ESI-pos) M+1=560.4

Step J: 10% w/w Palladium on carbon (13.5 mg, 12.7 μmol) was added to a solution of tert-butyl (8S,22Z)-24,24-difluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,22,25,27-decaene-7-carboxylate (14.2 mg, 25.4 μmol) in methanol (1 mL). The mixture was sparged with hydrogen for 1 minute and stirred under a balloon of hydrogen for 1 hour at room temperature. The reaction filtered through Celite with DCM and concentrated in vacuo to afford tert-butyl (S)-24,24-difluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (14.3 mg, 25.5 μmol). m/z (ESI-pos) M+1=562.4

Step K: 4M HCl in dioxane (0.5 mL) was added to a stirred solution of tert-butyl (S)-24,24-difluoro-8-methyl-30-oxo-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaene-7-carboxylate (14.3 mg, 25.5 μmol) in dichloromethane (0.5 mL) at room temperature. The solution was stirred at room temperature for 80 minutes and concentrated in vacuo to provide(S)-24,24-difluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (12.7 mg, 25.5 μmol). m/z (ESI-pos) M+1=462.3.

Step L: To a solution of (S)-24,24-difluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one-hydrogen chloride (1/1) (12.7 mg, 25.5 μmol), 5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolecarboxylic acid (13.6 mg, 33.2 μmol), and DIEA (22.2 μL, 128 μmol) in dimethylformamide (0.6 mL) was added HATU (19.4 mg, 51 μmol) at room temperature. The reaction was stirred at room temperature for 2 hours. The reaction mixture was diluted with sat. aq. NH₄Cl and EtOAc. The organic layer was washed with water, washed with brine, dried over anhydrous Na₂SO₄, filtered, and concentrated in vacuo. The crude residue was purified by reverse-phase chromatography (40-100% MeCN in water with 0.1% FA) to provide(S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-24,24-difluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one (6.6 mg, 7.6 μmol) as a white solid. m/z (ESI-pos) M+1=855.5; ¹H NMR (400 MHz, CHLOROFORM-D) δ 11.34-11.16 (m, 1H), 7.84-7.69 (m, 1H), 7.65-7.49 (m, 3H), 7.49-7.38 (m, 1H), 7.38-7.13 (m, 6H), 6.81-6.68 (m, 1H), 6.57-6.07 (m, 2H), 5.90-5.21 (m, 1H), 4.98-4.41 (m, 1H), 3.95-3.75 (m, 2H), 3.71-3.31 (m, 1H), 3.27-2.95 (m, 3H), 2.87-2.18 (m, 2H), 1.98-1.84 (m, 1H), 1.84-1.39 (m, 10H), 1.39-1.01 (m, 12H).

### Example 239—Synthesis of Compound 239

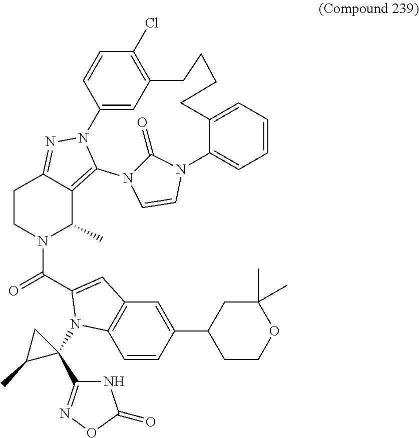

### (S)-26-chloro-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 239 was synthesized following the procedure of Example 34, (3-bromo-4-fluorophenyl)hydrazine being replaced with (3-bromo-4-chlorophenyl)hydrazine in step B. m/z (ESI-pos) M+1=853.6; ¹H NMR (400 MHz, CHLOROFORM-D) δ 11.34-11.16 (m, 1H), 7.64-7.40 (m, 3H), 7.39-7.12 (m, 7H), 6.79-6.67 (m, 1H), 6.49-5.90 (m, 2H), 5.89-5.17 (m, 1H), 4.99-4.19 (m, 1H), 3.99-3.73 (m, 2H), 3.72-2.32 (m, 8H), 2.03-1.45 (m, 11H), 1.44-0.96 (m, 12H).

### Example 240—Synthesis of Compound 240

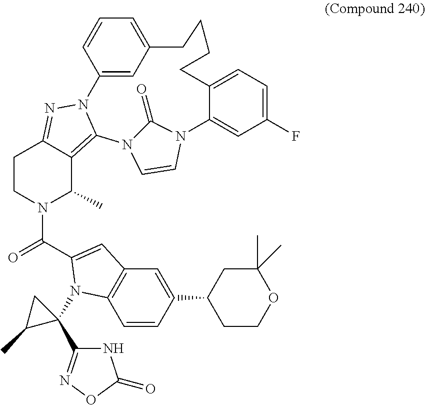 [SMILES: C[C@H]1c2c(nn3c2-n2ccn(c2=O)-c2cc(F)ccc2CCCCc2cccc-3c2)CCN1C(=O)c1cc2cc([C@H]3CCOC(C)(C)C3)ccc2n1[C@@]1(c2noc(=O)[nH]2)C[C@@H]1C]

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-17-fluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 240 was synthesized following the procedure of Example 34, (3-bromo-4-fluorophenyl)hydrazine being replaced with (m-bromophenyl)hydrazine in step B and 1-allyl-2-bromobenzene being replaced with 1-allyl-2-bromo-4-fluorobenzene in step G. m/z (ESI-pos) M+1=837.6; ¹H NMR (400 MHz, CHLOROFORM-D) δ 11.37-11.16 (m, 1H), 7.64-7.38 (m, 4H), 7.33-6.88 (m, 6H), 6.80-6.66 (m, 1H), 6.42-5.90 (m, 2H), 5.89-5.17 (m, 1H), 4.95-4.38 (m, 1H), 3.95-3.75 (m, 2H), 3.73-2.89 (m, 5H), 2.83-2.60 (m, 2H), 2.51-2.22 (m, 1H), 1.97-0.99 (m, 23H).

### Example 241—Synthesis of Compound 241

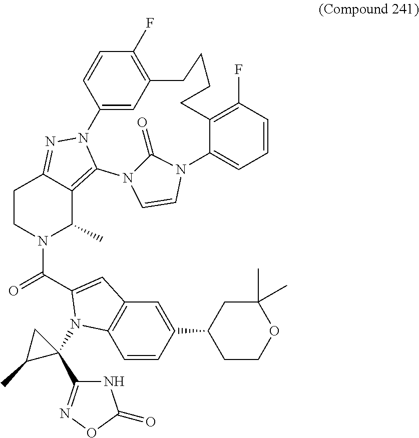

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-19,26-difluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 241 was synthesized following the procedure of Example 34, 1-allyl-2-bromobenzene being replaced with 2-allyl-1-bromo-3-fluorobenzene in step G. m/z (ESI-pos) M+1=855.6; ¹H NMR (400 MHz, CHLOROFORM-D) δ 11.31-11.19 (m, 1H), 7.64-7.38 (m, 4H), 7.34-6.94 (m, 5H), 6.79-6.66 (m, 1H), 6.45-5.90 (m, 2H), 5.90-5.16 (m, 1H), 4.98-4.38 (m, 1H), 3.96-3.76 (m, 2H), 3.73-2.53 (m, 8H), 1.95-1.48 (m, 11H), 1.43-1.00 (m, 12H).

### Example 242—Synthesis of Compound 242

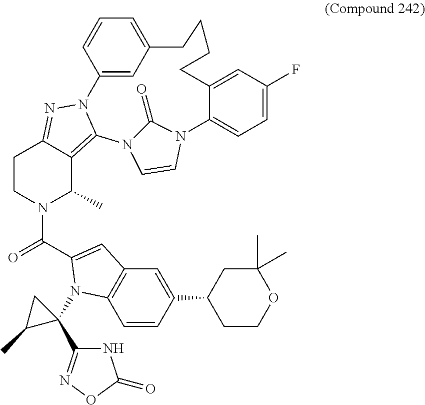 [SMILES: C[C@H]1c2c(nn3c2-n2ccn(c2=O)-c2ccc(F)cc2CCCCc2cccc-3c2)CCN1C(=O)c1cc2cc([C@H]3CCOC(C)(C)C3)ccc2n1[C@@]1(c2noc(=O)[nH]2)C[C@@H]1C]

### (S)-7-({5-[(S)-2,2-dimethyltetrahydro-4H-pyran-4-yl]-1-[(1S,2S)-2-methyl-1-(5-oxo-4,5-dihydro-1,2,4-oxadiazol-3-yl)cyclopropyl]-2-indolyl}carbonyl)-18-fluoro-8-methyl-2,3,7,11,14-pentaazahexacyclo[23.3.1.1¹¹,¹⁴.0²,¹⁰.0⁴,⁹.0¹⁵,²⁰]triaconta-1(29),3,9,12,15,17,19,25,27-nonaen-30-one

Compound 242 was synthesized following the procedure of Example 34, (3-bromo-4-fluorophenyl)hydrazine being replaced with (m-bromophenyl)hydrazine in step B and 1-allyl-2-bromobenzene being replaced with 2-allyl-1-bromo-4-fluorobenzene in step G. m/z (ESI-pos) M+1=837.6; ¹H NMR (400 MHz, CHLOROFORM-D) δ 11.36-11.20 (m, 1H), 7.64-7.37 (m, 3H), 7.35-6.82 (m, 7H), 6.79-6.66 (m, 1H), 6.40-5.89 (m, 2H), 5.88-5.15 (m, 1H), 4.97-4.38 (m, 1H), 3.94-3.75 (m, 2H), 3.73-2.17 (m, 8H), 1.97-1.45 (m, 11H), 1.44-1.00 (m, 12H).

### Cell Assay Principle

In vitro potency of the agonists described herein was determined by monitoring the production of cyclic adenosine monophosphate (CAMP) in CHO-K1 cells stably expressing human glucagon-like peptide-1 receptor (hGLP1R). Upon binding of the incretin GLP1 or other small molecule agonist to the receptor and the downstream exchange of GDP for GTP, the Gas subunit of the heterotrimeric G-protein complex dissociates. Active Gas recruits adenyl cyclase which then converts adenosine triphosphate (ATP) to CAMP which in turn acts as a secondary messenger to affect downstream biological processes through Protein Kinase A (PKA). Production of CAMP is monitored using a homogeneous, time-resolved fluorescence (HTRF) based assay where native cAMP produced by the cells and d2-labeled CAMP compete for a monoclonal anti-cAMP antibody conjugated to a europium cryptate. The amount of fluorescence produced is inversely proportional to the concentration of cAMP generated by the receptor/agonist interaction in vitro.

Cellular GLP-1R Agonist cAMP-Assay A

Test compounds were dissolved in dimethyl sulfoxide (DMSO) to 1000× the desired final assay concentration followed by a 10-point, 3-fold serial dilution in DMSO. Using an Echo acoustic liquid handler, 10 nL was transferred to a white, polystyrene 384-well assay plate (Greiner Cat. 784075). GLP1 (7-37) (MedChemExpress Cat. HY-P0055) was used as a positive, agonist control using a concentration range from 1 nM to 51 fM.

HEK293 cells stably overexpressing hGLPIR (BioDuro) were prepared and resuspended in Dulbecco's Phosphate-Buffered Saline (DPBS) (Gibco Cat. 14190-144) containing 0.5% bovine serum albumin (BSA) (MERCK Cat. V900933) and 500 μM 3-isobutyl-1-methylxanthine (IBMX, MedChemExpress Cat. HY-12318) to a final density of 100,000 cells/mL. 10 μL was then transferred to each assay plate, centrifuged (1 minute @ 1,000 RPM) and incubated at room temperature (23° C.) for 30 minutes to a final plated density of 1,000 cells/well and 0.1% DMSO (v/v). After this incubation, 5 L of both CAMP-d2 and 5 μL of anti-cAMP Europium conjugated antibody in detection buffer (Revvity CAMP Gs Dynamic Detection Kit, Cat. 62AM4PEC) were added followed by a room temperature incubation for 60 minutes. After this secondary incubation, each assay plate was read using an Envision Multimode Reader (Revvity) at Ex.340 nm and Em.665 nm/Em.620 nm.

Cellular GLP-1R Agonist cAMP-Assay B

CHO-K1 cells stably overexpressing hGLPIR (Eurofins/DiscoverX Cat. 95-0062C2) were prepared and resuspended in Dulbecco's Phosphate-Buffered Saline (DPBS) (Gibco Cat. 14040-133) containing 0.1% bovine serum albumin (BSA) (Fisher Bioreagents Cat. BP9706) and 500 μM 3-isobutyl-1-methylxanthine (IBMX, MedChemExpress Cat. HY-12318) to a final density of 125,000 cells/mL. 8 μL was then transferred to a white, polystyrene 384-well assay plate (Greiner Cat. 784075) to a final plated density of 1,000 cells/well.

Test compounds were dissolved in dimethyl sulfoxide (DMSO) to 500× the desired final assay concentration followed by a 10-point, 4-fold serial dilution in DMSO. Compounds were then diluted in assay buffer and 2 μL was transferred to each assay plate using an Apricot PP5 Personal Pipettor liquid handler to 0.1% DMSO (v/v). A fixed concentration of Danuglipron (MedChemExpress Cat. HY-125824) was used as a positive agonist control and the absence of agonist used as a negative control. Assay plates were then centrifuged (1 minute @ 500 RPM) and incubated at 37° C. for 60 minutes.

After this incubation, 5 μL of both cAMP-d2 and 5 μL of anti-cAMP Europium conjugated antibody in detection buffer (Revvity CAMP Gs Dynamic Detection Kit, Cat. 62AM4PEC) were added followed by a room temperature incubation for 60 minutes. After this secondary incubation, each assay plate was read using an Envision Multimode Reader (Revvity) at Ex.340 nm and Em.665 nm/Em.615 nm.

### Data Analysis

Data were analyzed by taking the ratio of Em.665/Em.615 at each test concentration and converting to the amount of cAMP produced through a standard curve located on each test plate. The following equation was then applied to determine % agonism (R) and then by fitting the converted dataset to a 4-parameter dose-response model in CDD Vault.

$$\% Agonism ( R ) = \frac{( cAMP {Produced}_{Test} ) - {\overline{X}}_{min}}{{\overline{X}}_{max} - {\overline{X}}_{min}} \times 100$$

### Where:

X̅ₘₐₓ=Average of the CAMP produced by the positive controls (maximal signal) defined as containing a known concentration of agonist in 0.1% DMSO
X̅ₘᵢₙ=Average of the CAMP produced by the negative controls (minimal signal) defined as containing 0.1% DMSO only

The EC₅₀ value was reported as the concentration of agonist where 50% overall response—as measured by cAMP production—between Rₘᵢₙ and Rₘₐₓ was observed. Biological activity data for representative compounds of the invention are provided in the Table below.

TABLE 1
Results of Cellular GLP-1R agonist cAMP assay

| | Assay A | Assay B |
|---|---|---|
| | EC₅₀ | EC₅₀ |
| Example No. | (nM) | (nM) |
| 1-Isomer 1 | 1.5 | 0.19 |
| 1-Isomer 2 | 55 | 2.92 |
| 2-Isomer 1 | 265 | 442 |
| 2-Isomer 2 | 1.7 | 0.16 |
| 3-Isomer 1 | | 0.71 |
| 3-Isomer 2 | | >500 |
| 4-Isomer 1 | 86 | 20 |
| 4-Isomer 2 | 0.85 | 0.08 |
| 5-Isomer 1 | 25 | 0.92 |
| 5-Isomer 2 | 1.6 | 0.06 |
| 6-Isomer 1 | 2.9 | 2.5 |
| 6-Isomer 2 | 3.1 | 2.5 |
| 7-Isomer 1 | 34 | 14.1 |
| 7-Isomer 2 | 12.8 | 7.1 |
| 8-Isomer 1 | 13.6 | 1.3 |
| 8-Isomer 2 | >1,000 | 609 |
| 9-Isomer 1 | | >1,000 |
| 9-Isomer 2 | | 3.3 |
| 10-Isomer 1 | | 155 |
| 10-Isomer 2 | | >1,000 |
| 11-Isomer 1 | 50 | |
| 11-Isomer 2 | 2.8 | |
| 12-Isomer 1 | | 4.7 |
| 12-Isomer 2 | | 67.4 |
| 13-Isomer 1 | | 542 |
| 13-Isomer 2 | | 374 |
| 14-Isomer 1 | 340 | 519 |
| 14-Isomer 2 | 212 | >1,000 |
| 15-Isomer 1 | | 1.4 |
| 15-Isomer 2 | | 128 |
| 16 | | 2.25 |
| 17 | | 2.9 |
| 18-Isomer 1 | | >500 |
| 18-Isomer 2 | | 6.05 |
| 19-Isomer 1 | | 0.52 |
| 19-Isomer 2 | | 173 |
| 20-Isomer 1 | | >900 |
| 20-Isomer 2 | | 7.2 |
| 21-Isomer 1 | | 4.86 |
| 21-Isomer 2 | | >1000 |
| 22-Isomer 1 | | 8.62 |
| 22-Isomer 2 | | 91.7 |
| 23 | | 0.71 |
| 24 | | 0.72 |
| 25 | | 0.44 |
| 26 | | 23.1 |
| 27 | | 3.9 |
| 28 | | 0.52 |
| 29 | | 1.4 |
| 30 | | 1.4 |
| 31 | | 2 |
| 32 | | 0.03 |
| 33-Isomer 1 | | 7.9 |
| 33-Isomer 2 | | 11 |
| 34 | | 1.8 |
| 35 | | 1.6 |
| 36-Isomer 1 | | 0.86 |
| 36-Isomer 2 | | 0.18 |
| 37 | | 3.1 |
| 38 | | 5.1 |
| 39 | | 0.17 |
| 40-Isomer 1 | | 14.7 |
| 40-Isomer 2 | | 0.3 |
| 41-Isomer 1 | | 38.8 |
| 41-Isomer 2 | | 0.14 |
| 42-Isomer 1 | | 32.1 |
| 42-Isomer 2 | | 0.85 |
| 43-Isomer 1 | | >1,000 |
| 43-Isomer 2 | | 1.9 |
| 44-Isomer 1 | | 0.04 |
| 44-Isomer 2 | | 27.4 |
| 45-Isomer 1 | | 386 |
| 45-Isomer 2 | | 0.26 |
| 46-Isomer 1 | | 1.9 |
| 46-Isomer 2 | | >1,000 |
| 47-Isomer 1 | | 2.2 |
| 47-Isomer 2 | | 35.3 |
| 48-Isomer 1 | | 134 |
| 48-Isomer 2 | | 0.28 |
| 49-Isomer 1 | | >1,000 |
| 49-Isomer 2 | | 2.5 |
| 50-Isomer 1 | | 48.3 |
| 50-Isomer 2 | | 0.93 |
| 51 | | 0.1 |
| 52 | | 0.37 |
| 53 | | 2.1 |
| 54 | | 0.39 |
| 55 | | 2.6 |
| 56 | | 0.46 |
| 57 | | 0.53 |
| 58 | | 1.2 |
| 59 | | 0.15 |
| 60 | | 0.21 |
| 61 | | 0.41 |
| 62 | | 0.87 |
| 63 | | 1.3 |
| 64 | | 0.49 |
| 65 | | 2.53 |
| 66 | | 0.05 |
| 67 | | 1.22 |
| 68 | | 0.03 |
| 69 | | 0.1 |
| 70 | | 0.49 |
| 71 | | 0.09 |
| 72 | | 0.04 |
| 73 | | 0.04 |
| 74 | | 0.03 |
| 75 | | 2.12 |
| 76 | | 0.1 |
| 77 | | 0.09 |
| 78 | | 0.12 |
| 79 | | 0.09 |
| 80 | | 33 |
| 81 | | 0.09 |
| 82 | | 0.88 |
| 83 | | 0.48 |
| 84 | | 0.03 |
| 85 | | 0.15 |
| 86 | | 0.19 |
| 87 | | 0.01 |
| 88 | | 0.08 |
| 89 | | 0.07 |
| 90 | | 0.71 |
| 91 | | 0.65 |
| 92 | | 1 |
| 93 | | 9.54 |
| 94 | | 0.01 |
| 95 | | 7.4 |
| 96 | | 0.92 |
| 97 | | 2.48 |
| 98 | | 2.63 |
| 99 | | 12.7 |
| 100 | | 12.2 |
| 101 | | 11.4 |
| 102 | | 7.71 |
| 103 | | 0.4 |
| 104 | | 2 |
| 105 | | 0.1 |
| 106 | | 0.06 |
| 107 | | 0.43 |
| 108 | | 0.49 |
| 109 | | 0.07 |
| 110 | | 6.94 |
| 111 | | 0.36 |
| 112 | | 0.33 |
| 113 | | 0.03 |
| 114 | | 0.19 |
| 115 | | 14.8 |
| 116 | | 0.07 |
| 117 | | 0.1 |
| 118 | | 0.1 |
| 119 | | 0.2 |
| 120 | | 0.02 |
| 121 | | 0.19 |
| 122 | | 0.52 |
| 123 | | 0.1 |
| 124 | | 0.05 |
| 125 | | 0.06 |
| 126 | | 0.04 |
| 127 | | 0.18 |
| 128 | | 0.05 |
| 129 | | 0.07 |
| 130 | | 0.02 |
| 131 | | 0.19 |
| 132 | | 0.09 |
| 133 | | 0.08 |
| 134 | | 0.09 |
| 135 | | 0.17 |
| 136 | | 0.02 |
| 137 | | 0.03 |
| 138 | | 0.05 |
| 139 | | 0.05 |
| 140 | | 0.06 |
| 141 | | 4.28 |
| 142 | | 0.61 |
| 143 | | 0.54 |
| 144 | | 0.86 |
| 145 | | 0.46 |
| 146 | | 0.3 |
| 147 | | 0.29 |
| 148 | | 19.5 |
| 149 | | 0.07 |
| 150 | | 1.32 |
| 151 | | 0.08 |
| 152 | | 0.13 |
| 153 | | 3.05 |
| 154 | | 0.15 |
| 155 | | 0.69 |
| 156 | | 0.13 |
| 157 | | 0.11 |
| 158 | | 0.1 |
| 159 | | 0.15 |
| 160 | | 0.23 |
| 161 | | 0.73 |
| 162 | | 0.92 |
| 163 | | 0.8 |
| 164 | | 0.06 |
| 165 | | 0.01 |
| 166 | | 0.02 |
| 167 | | 0.03 |
| 168 | | 0.07 |
| 169 | | 0.05 |
| 170 | | 0.06 |
| 171 | | 0.06 |
| 172 | | 0.04 |
| 173 | | 0.26 |
| 174 | | 0.03 |
| 175 | | 0.09 |
| 176 | | 2.44 |
| 177 | | 0.35 |
| 178 | | 0.84 |
| 179 | | 0.09 |
| 180 | | 0.9 |
| 181 | | 0.88 |
| 182 | | 2.46 |
| 183 | | 0.49 |
| 184 | | 0.35 |
| 185 | | 0.23 |
| 186 | | 0.1 |
| 187 | | 0.36 |
| 188 | | 14 |
| 189 | | 0.3 |
| 190 | | 2.16 |
| 191 | | 0.17 |
| 192 | | 0.11 |
| 193 | | 0.16 |
| 194 | | 0.1 |
| 195 | | 0.08 |
| 196 | | 0.31 |
| 197 | | 3.69 |
| 198 | | 0.09 |
| 199 | | 0.03 |
| 200 | | 0.12 |
| 201 | | 0.05 |
| 202 | | 0.49 |
| 203 | | 0.25 |
| 204 | | 0.48 |
| 205 | | 0.4 |
| 206 | | 0.03 |
| 207 | | 0.08 |
| 208 | | 0.06 |
| 209 | | 0.05 |
| 210 | | 0.05 |
| 211 | | 0.13 |
| 212 | | 0.07 |
| 213 | | 0.02 |
| 214 | | 0.08 |
| 215 | | 0.08 |
| 216 | | 0.08 |
| 217 | | 0.09 |
| 218 | | 0.11 |
| 219 | | 0.44 |
| 220 | | 0.16 |
| 221 | | 0.04 |
| 222 | | 0.07 |
| 223 | | 0.26 |
| 224 | | 0.31 |
| 225 | | 38 |
| 226 | | 0.41 |
| 227 | | 0.49 |
| 228 | | 0.47 |
| 229 | | 6.64 |
| 230 | | 0.27 |
| 231 | | 0.37 |
| 232 | | 0.97 |
| 233 | | 0.45 |
| 234 | | 0.48 |
| 235 | | 0.07 |
| 236 | | 0.73 |
| 237 | | 1.31 |
| 238 | | 3.69 |
| 239 | | 2.89 |
| 240 | | 1.98 |
| 241 | | 1.89 |
| 242 | | 1.65 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of formula (I): 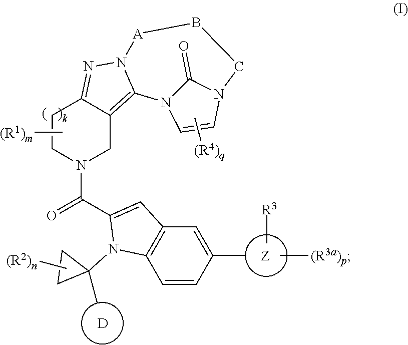
or a pharmaceutically acceptable salt thereof; wherein,
A is optionally substituted arylene, heteroarylene, or heterocycloalkylene;
B is -(alkylene)O(alkylene)-, alkylene, haloalkylene, alkenylene, —O(alkylene)-, -(alkylene)O—, —O(alkylene)O—, —O(alkylene)O(alkylene)-, -(alkylene)O(alkylene)O—, —S(alkylene)-, -(alkylene)S—, -(alkylene)S(alkylene)-, —S(alkylene)S—, —S(alkylene)S(alkylene)-, -(alkylene)S(alkylene)S—, —NH(alkylene)-, -(alkylene)NH—, -(alkylene)NH(alkylene)-, —NH(alkylene)NH—, —NH(alkylene)NH(alkylene)-, -(alkylene)NH(alkylene)NH—, —N(alkyl)(alkylene)-, -(alkylene)N(alkyl)-, or is a bond;
C is optionally substituted arylene, heteroarylene, or heterocycloalkylene;
Ring D is 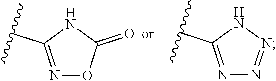
Ring Z is heterocycloalkylene or heteroarylene;
R¹ is independently for each occurrence alkyl, alkoxy, or halo; or two geminal occurrences of R¹ taken together with the carbon atom to which they are attached form an optionally substituted spirocyclic cycloalkyl ring;
R² is independently for each occurrence alkyl, alkoxy, or halo;
R³ is haloalkyl, unsubstituted cycloalkyl, cycloalkyl substituted with alkyl, cycloalkyl substituted with haloalkyl, cycloalkyl substituted with halo, unsubstituted heteroaryl, heteroaryl substituted with alkyl, heteroaryl substituted with halo, unsubstituted heterocycloalkyl, heterocycloalkyl substituted with alkyl, heterocycloalkyl substituted with halo, haloalkoxy, —NH₂, —NH(alkyl), or —N(alkyl)₂;
R^{3a} is independently for each occurrence alkyl, alkoxy, halo, haloalkyl, optionally substituted cycloalkyl, optionally substituted heteroaryl, optionally substituted heterocycloalkyl, haloalkoxy, —NH₂, —NH(alkyl), or —N(alkyl)₂;
R⁴ is independently for each occurrence alkyl, alkoxy, or halo;
k is an integer selected from 0-2;
m is an integer selected from 0-6;
n is an integer selected from 0-4;
p is an integer selected from 0-4; and
q is an integer selected from 0-2.

2-9. (canceled)

10. The compound of claim 1, wherein the compound is represented by formula (I-h): 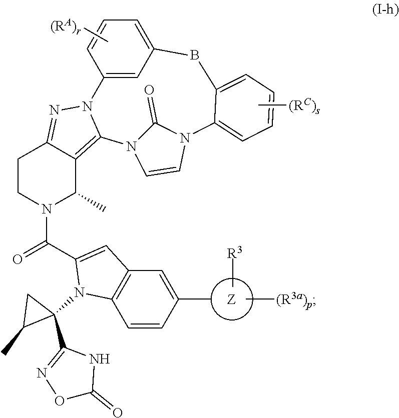
or a pharmaceutically acceptable salt thereof; wherein,
R^{A} is independently for each occurrence alkyl, cycloalkyl, heterocycloalkyl, alkenyl, cycloalkenyl, heterocycloalkenyl, alkynyl, silyl, —NH₂, —NH(alkyl), —N(alkyl)₂, hydroxyl, hydroxyalkyl, alkoxy, —SH, —S-alkyl, —S—S-alkyl, halo, haloalkyl, haloalkoxy, —C(O)H, —C(O)-alkyl, —C(O)NH₂, —C(O)NH(alkyl), —C(O)N(alkyl)₂,
—C(O)N(alkyl)(cycloalkyl), —C(O)OH, —OC(O)-alkyl, —C(O)O-alkyl, —C(O)S-alkyl, —S(O)-alkyl, —S(O)₂-alkyl, —S(O)₂-cycloalkyl, —S(O)₂NH₂, —S(O)₂NH(alkyl), —S(O)₂N(alkyl)₂, —S(O)NH₂, —S(O)NH(alkyl), —S(O)N(alkyl)₂, —S(═O)(═NH)(-alkyl), —S(═O)(═N-alkyl)(-alkyl), —CH₂S(O)₂ (alkyl), —OP(O)(O-alkyl)₂, —P(O)(alkyl)₂, —CN, —NO₂, —N₃, aryl, or heteroaryl;
R^{C} is independently for each occurrence alkyl, cycloalkyl, heterocycloalkyl, alkenyl, cycloalkenyl, heterocycloalkenyl, alkynyl, silyl, —NH₂, —NH(alkyl), —N(alkyl)₂, hydroxyl, hydroxyalkyl, alkoxy, —SH, —S-alkyl, —S—S-alkyl, halo, haloalkyl, haloalkoxy, —C(O)H, —C(O)-alkyl, —C(O)NH₂, —C(O)NH(alkyl), —C(O)N(alkyl)₂,
—C(O)N(alkyl)(cycloalkyl), —C(O)OH, —OC(O)-alkyl, —C(O)O-alkyl, —C(O)S-alkyl, —S(O)-alkyl, —S(O)₂-alkyl, —S(O)₂-cycloalkyl, —S(O)₂NH₂, —S(O)₂NH(alkyl), —S(O)₂N(alkyl)₂, —S(O)NH₂, —S(O)NH(alkyl), —S(O)N(alkyl)₂, —S(═O)(═NH)(-alkyl), —S(═O)(═N-alkyl)(-alkyl), —CH₂S(O)₂ (alkyl), —OP(O)(O-alkyl)₂, —P(O)(alkyl)₂, —CN, —NO₂, —N₃, aryl, or heteroaryl;
r is an integer selected from 0-4; and
s is an integer selected from 0-4.

11. The compound of claim 1, wherein A is optionally substituted phenylene, pyridinylene, pyridazinylene, pyrimidinylene, or pyrazinylene.

12. (canceled)

13. The compound of claim 1, wherein A is optionally substituted by one or more R^{A}, wherein R^{A} is independently for each occurrence alkyl, cycloalkyl, heterocycloalkyl, alkenyl, cycloalkenyl, heterocycloalkenyl, alkynyl, silyl, —NH₂, —NH(alkyl), —N(alkyl)₂, hydroxyl, hydroxyalkyl, alkoxy, —SH, —S-alkyl, —S—S-alkyl, halo, haloalkyl, haloalkoxy, —C(O)H, —C(O)-alkyl, —C(O)NH₂, —C(O)NH(alkyl), —C(O)N(alkyl)₂, —C(O)N(alkyl)(cycloalkyl), —C(O)OH, —OC(O)-alkyl, —C(O)O-alkyl, —C(O)S-alkyl, —S(O)-alkyl, —S(O)₂-alkyl, —S(O)₂-cycloalkyl, —S(O)₂NH₂, —S(O)₂NH(alkyl), —S(O)₂N(alkyl)₂, —S(O)NH₂, —S(O)NH(alkyl), —S(O)N(alkyl)₂, —S(═O)(═NH)(-alkyl), —S(═O)(═N-alkyl)(-alkyl), —CH₂S(O)₂ (alkyl), —OP(O)(O-alkyl)₂, —P(O)(alkyl)₂, —CN, —NO₂, —N₃, aryl, or heteroaryl.

14. (canceled)

15. The compound of claim 13, wherein each RA is independently methyl, trifluoromethyl, cyclopropyl, methoxy, fluoro, or chloro.

16-18. (canceled)

19. The compound of claim 1, wherein -A-* is 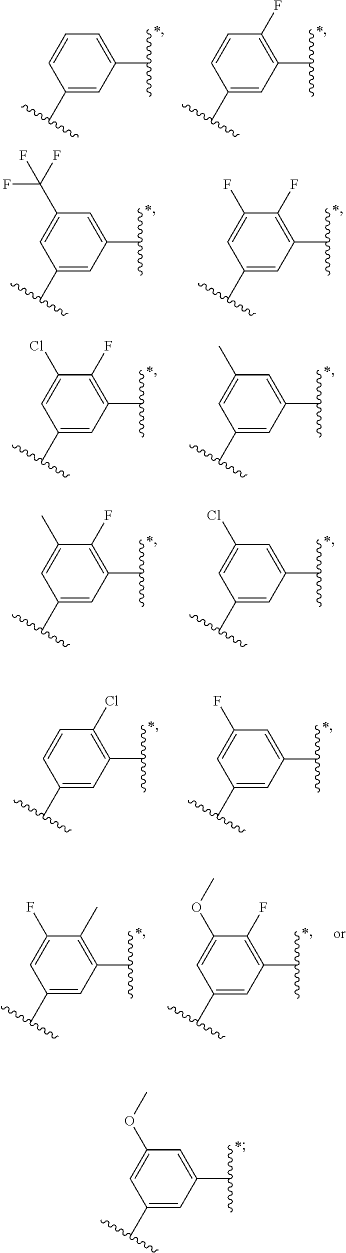
wherein the asterisk (*) indicates the point of attachment to B.

20. (canceled)

21. The compound of claim 1, wherein B is
—CH₂CH₂—, *—CH₂CH₂CH₂—, *—CH₂CH₂CH₂CH₂—, *—CH₂CH₂CH₂CH₂CH₂—, *—CF₂CH₂CH₂CH₂—, *—CH(CH₃)OCH₂—, *—OCH₂—, *—CH₂O—, *—OCH₂CH₂—, *—CH₂OCH₂—, *—CH₂CH₂O—, *—OCH₂CH₂CH₂—, *—CH₂OCH₂CH₂—, *—CH₂CH₂OCH₂—, *—CH₂CH₂CH₂O—, *—OCH₂CH₂O—, *—OCH₂CH₂CH₂CH₂—, *—CH₂OCH₂CH₂CH₂—, *—CH₂CH₂OCH₂CH₂—, *—CH₂CH₂CH₂OCH₂—, *—CH₂CH₂CH₂CH₂O—, *—SCH₂—, *—CH₂S—, *—SCH₂CH₂—, *—CH₂SCH₂—, *—CH₂CH₂S—, *—SCH₂CH₂CH₂—, *—CH₂SCH₂CH₂—, *—CH₂CH₂SCH₂—, *—CH₂CH₂CH₂S—, *—SCH₂CH₂S—, *—SCH₂CH₂CH₂CH₂—, *—CH₂SCH₂CH₂CH₂—, *—CH₂CH₂SCH₂CH₂—, *—CH₂CH₂CH₂SCH₂—, *—CH₂CH₂CH₂CH₂S—, *—CH—CH—, *—CH═CHCH₂—, *—CH₂CH═CH—, *—CH—CHCH₂CH₂—, *—CH₂CH═CHCH₂—, *—CH₂CH₂CH═CH—, *—CH═CHCH₂CH₂CH₂—, *—CH₂CH—CHCH₂CH₂—, *—CH₂CH₂CH—CHCH₂—, *—CH₂CH₂CH₂CH═CH—, *—N(CH₃)CH₂CH₂—, or *—CH₂CH₂N(CH₃)—;
wherein the asterisk (*) indicates the point of attachment to A.

22. (canceled)

23. The compound of claim 21, wherein B is 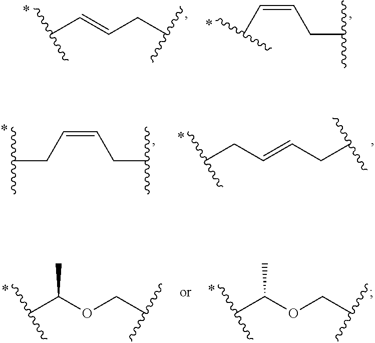
wherein the asterisk (*) indicates the point of attachment to A.

24. (canceled)

25. The compound of claim 1, wherein C is optionally substituted phenylene, pyridinylene, pyridazinylene, pyrimidinylene, pyrazinylene, indolylene, indazolylene, benzimidazolylene, or 2-hydroxybenzimidazolylene.

26. (canceled)

27. The compound of claim 25, wherein C is optionally substituted by one or more R^{C}, wherein R^{C} is independently for each occurrence alkyl, cycloalkyl, heterocycloalkyl, alkenyl, cycloalkenyl, heterocycloalkenyl, alkynyl, silyl, —NH₂, —NH(alkyl), —N(alkyl)₂, hydroxyl, hydroxyalkyl, alkoxy, —SH, —S-alkyl, —S—S-alkyl, halo, haloalkyl, haloalkoxy, —C(O)H, —C(O)-alkyl, —C(O)NH₂, —C(O)NH(alkyl), —C(O)N(alkyl)₂, —C(O)N(alkyl)(cycloalkyl), —C(O)OH, —OC(O)-alkyl, —C(O)O-alkyl, —C(O)S-alkyl, —S(O)-alkyl, —S(O)₂-alkyl, —S(O)₂-cycloalkyl, —S(O)₂NH₂, —S(O)₂NH(alkyl), —S(O)₂N(alkyl)₂, —S(O)NH₂, —S(O)NH(alkyl), —S(O)N(alkyl)₂, —S(═O)(═NH)(-alkyl), —S(═O)(═N-alkyl)(-alkyl), —CH₂S(O)₂ (alkyl), —OP(O)(O-alkyl)₂, —P(O)(alkyl)₂, —CN, —NO₂, —N₃, aryl, or heteroaryl.

28. (canceled)

29. The compound of claim 27, wherein each R^{C} is independently methyl, methoxy, chloro, fluoro, —C(O)NHMe, —S(O)₂Me, —S(O)₂Et, —CN,

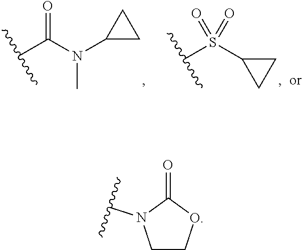

30-32. (canceled)

33. The compound of claim 1, wherein *—C— is 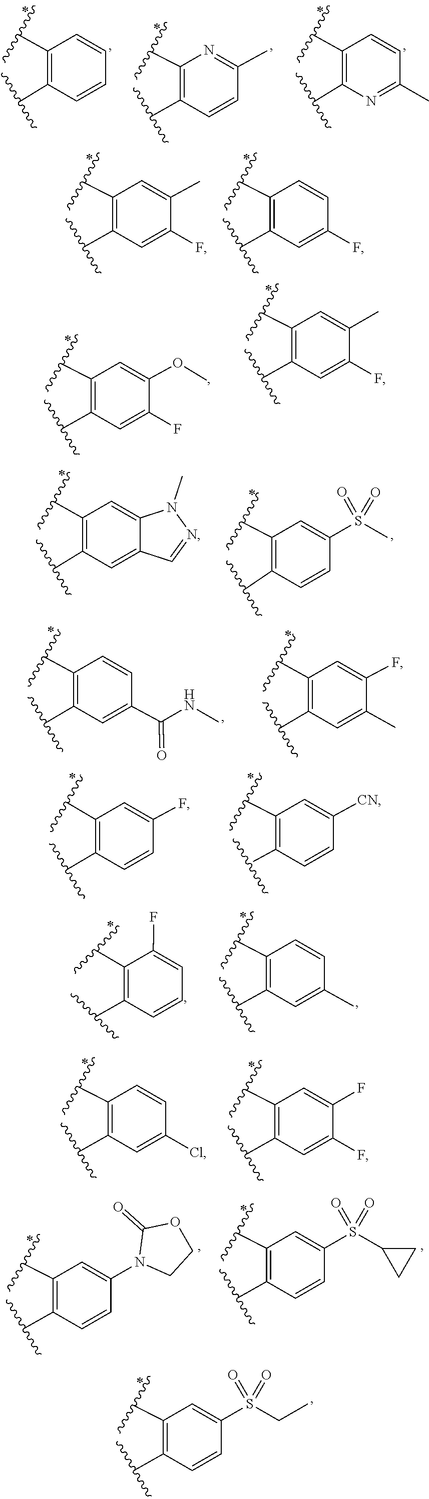 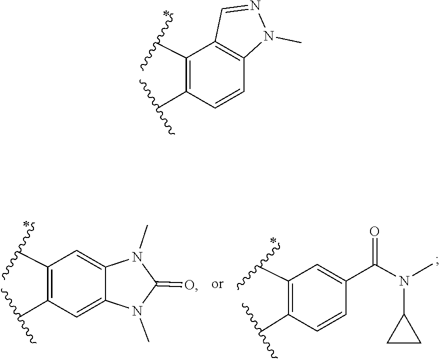 [SMILES: *c1ccc2c(cnn2C)c1* ; *c1cc2c(cc1*)n(C)c(=O)n2C ; "or" ; *c1ccc(C(=O)N(C)C2CC2)cc1*]
wherein the asterisk (*) indicates the point of attachment to B.

34. (canceled)

35. The compound of claim 1, wherein ring Z is heterocycloalkylene.

36. The compound of claim 35, wherein ring Z is tetrahydropyranylene, morpholinylene, 8-oxa-3-azabicyclo[3.2.1]octanylene, 2,3-dihydro-1H-pyrido[2,3-b][1,4]oxazinylene, 6,7-dihydro-5H-cyclopenta[b]pyridinylene, 2-oxa-6-azaspiro[3.3]heptanylene, or 1,4-oxazepanylene.

37. (canceled)

38. The compound of claim 1, wherein ring Z is heteroarylene.

39. The compound of claim 38, wherein ring Z is pyridinylene, pyrimidinylene, pyridazinylene, pyrazinylene, imidazolylene, 2,3-dihydro-1H-pyrido[2,3-b][1,4]oxazinylene, 6,7-dihydro-5H-cyclopenta[b]pyridinylene, 1H-pyrrolo[2,3-b]pyridinylene, 1,2,3-triazolylene, or pyrazolylene.

40. (canceled)

41. The compound of claim 1, wherein R³ is —NH₂, —N(Me) (Et), NMe₂,
—CHF₂, —CF₃, —CH₂CF₃, —CF₂CH₃, —CF₂CF₃, —OCHF₂, —OCF₃,

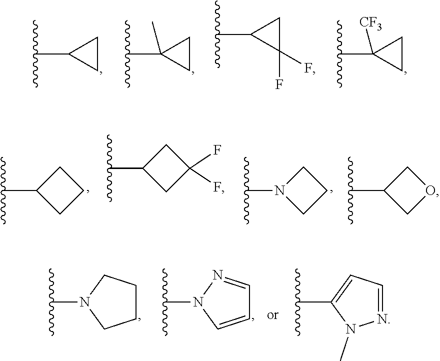

42. (canceled)

43. The compound of claim 1, wherein R^{3a} is alkyl or halo.

44. The compound of claim 43, wherein R^{3a} is methyl or fluoro.

45-46. (canceled)

47. The compound of claim 1, wherein

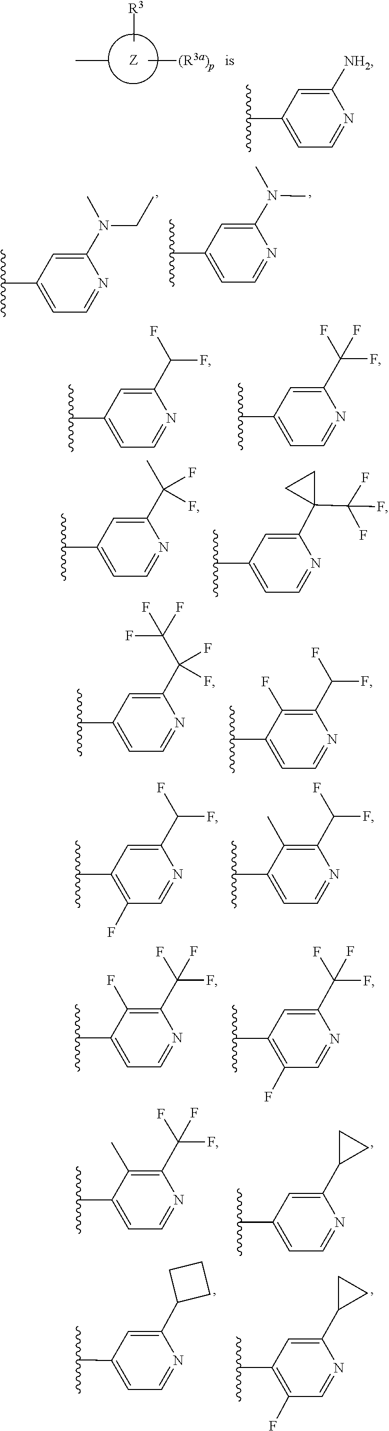

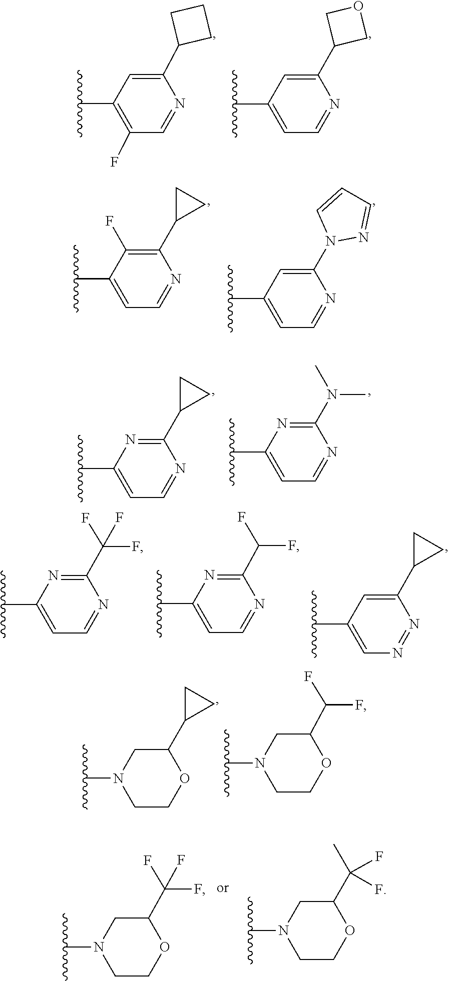

48. (canceled)

49. The compound of claim 1, wherein R¹ is alkyl.

50. The compound of claim 49, wherein R¹ is methyl.

51. (canceled)

52. The compound of claim 1, wherein two geminal occurrences of R¹ taken together with the carbon atom to which they are attached form a spirocyclic cyclopropyl ring.

53-55. (canceled)

56. The compound of claim 1, wherein R² is methyl.

57-59. (canceled)

60. The compound of claim 1, wherein the compound is selected from: 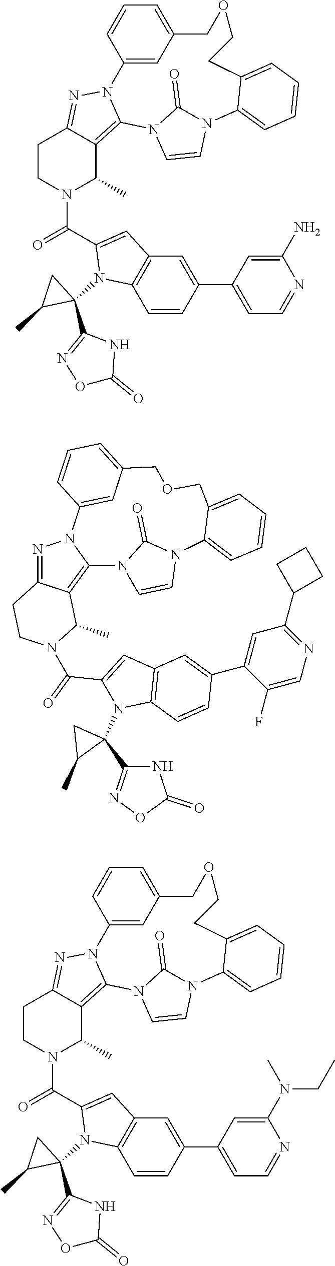 [SMILES: C[C@H]1c2c(nn3c2-n2ccn(c2=O)-c2ccccc2CCOCc2cccc-3c2)CCN1C(=O)c1cc2cc(-c3ccnc(N)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3cc(C4CCC4)ncc3F)ccc2n1[C@@]1(c2noc(=O)[nH]2)C[C@@H]1C ; CCN(C)c1cc(-c2ccc3c(c2)cc(C(=O)N2CCc4nn5c(c4[C@@H]2C)-n2ccn(c2=O)-c2ccccc2CCOCc2cccc-5c2)n3[C@@]2(c3noc(=O)[nH]3)C[C@@H]2C)ccn1] 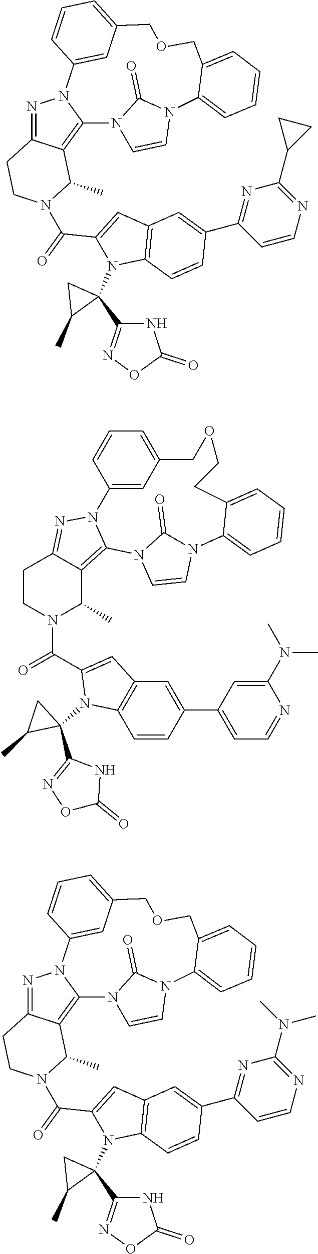 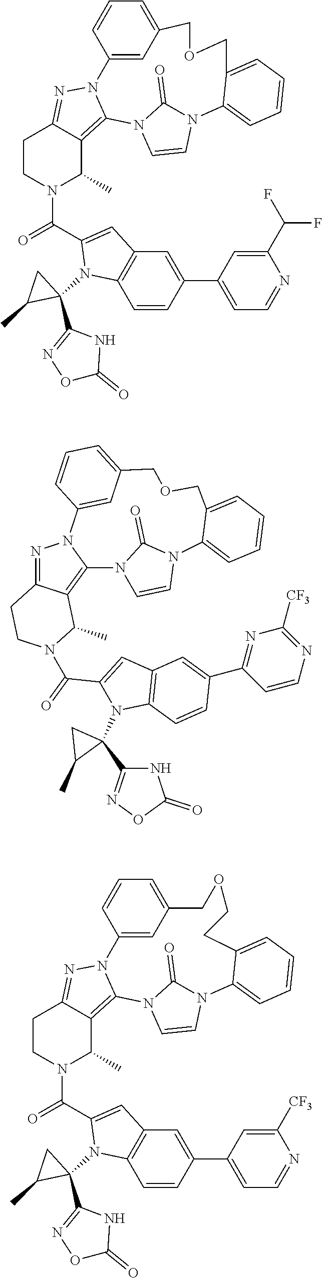 [SMILES: C[C@H]1c2c(nn3c2-n2ccn(c2=O)-c2ccccc2COCc2cccc-3c2)CCN1C(=O)c1cc2cc(-c3ccnc(C(F)F)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3ccnc(C(F)(F)F)n3)ccc2n1[C@@]1(c2noc(=O)[nH]2)C[C@@H]1C ; C[C@H]1c2c(nn3c2-n2ccn(c2=O)-c2ccccc2CCOCc2cccc-3c2)CCN1C(=O)c1cc2cc(-c3ccnc(C(F)(F)F)c3)ccc2n1[C@@]1(c2noc(=O)[nH]2)C[C@@H]1C] 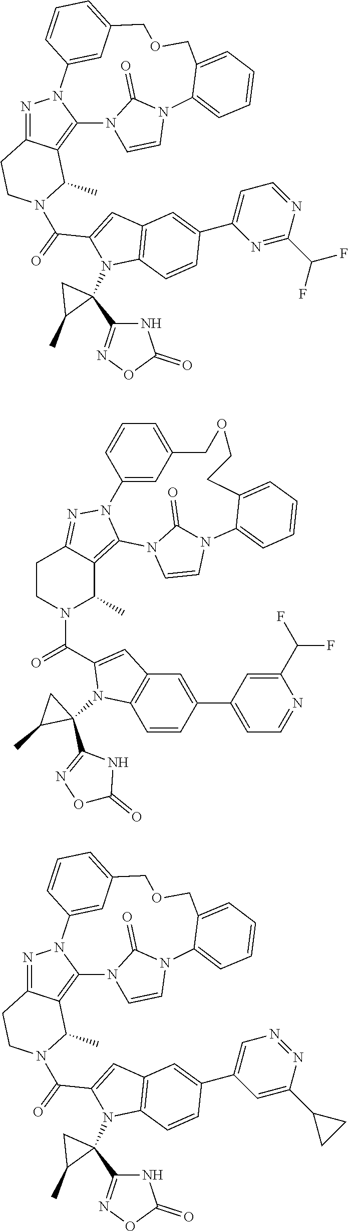 [SMILES: C[C@H]1c2c(nn3c2-n2ccn(c2=O)-c2ccccc2COCc2cccc-3c2)CCN1C(=O)c1cc2cc(-c3ccnc(C(F)F)n3)ccc2n1[C@@]1(c2noc(=O)[nH]2)C[C@@H]1C ; C[C@H]1c2c(nn3c2-n2ccn(c2=O)-c2ccccc2CCOCc2cccc-3c2)CCN1C(=O)c1cc2cc(-c3ccnc(C(F)F)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3cnnc(C4CC4)c3)ccc2n1[C@@]1(c2noc(=O)[nH]2)C[C@@H]1C] 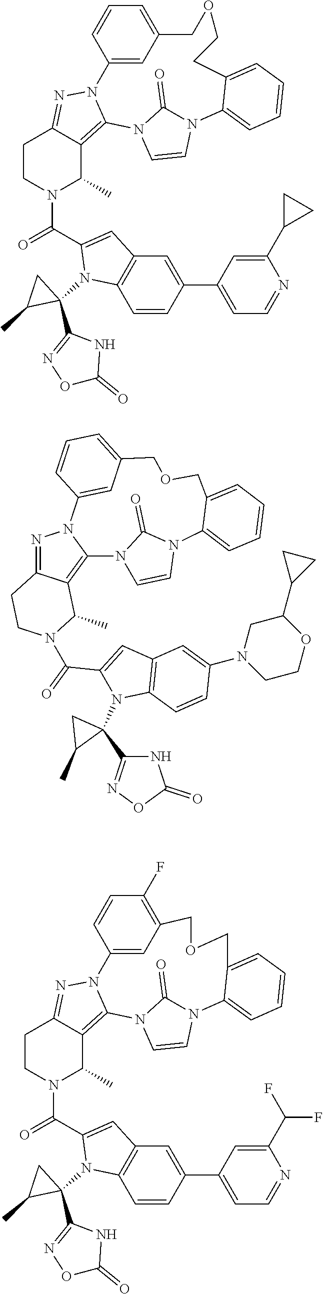 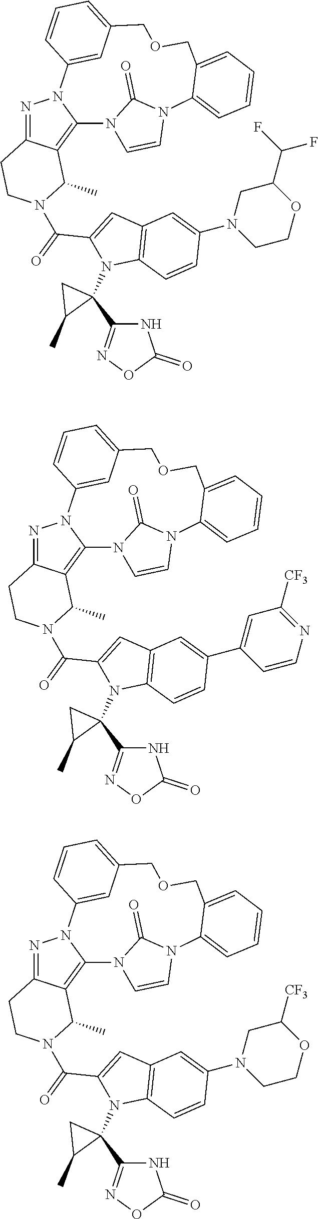 [SMILES: C[C@H]1c2c(nn3c2-n2ccn(c2=O)-c2ccccc2COCc2cccc-3c2)CCN1C(=O)c1cc2cc(N3CCOC(C(F)F)C3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3ccnc(C(F)(F)F)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(N3CCOC(C(F)(F)F)C3)ccc2n1[C@@]1(c2noc(=O)[nH]2)C[C@@H]1C] 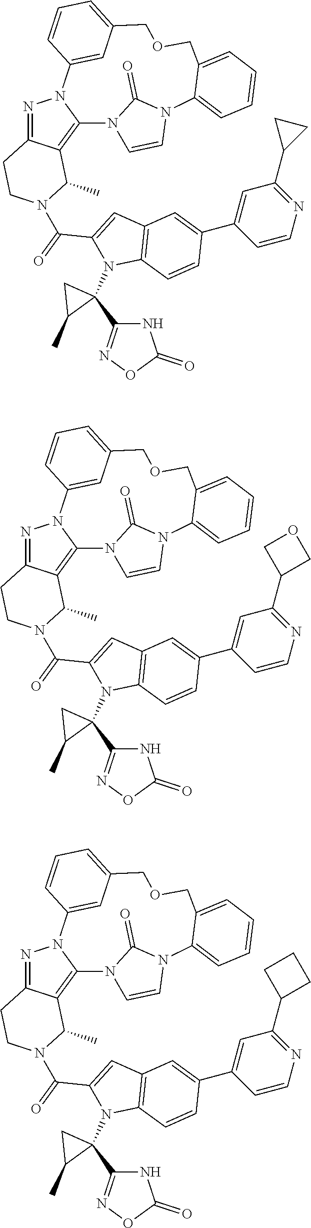 [SMILES: C[C@H]1c2c(nn3c2-n2ccn(c2=O)-c2ccccc2COCc2cccc-3c2)CCN1C(=O)c1cc2cc(-c3ccnc(C4CC4)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3ccnc(C4COC4)c3)ccc2n1[C@@]1(c2noc(=O)[nH]2)C[C@@H]1C ; C[C@H]1c2c(nn3c2-n2ccn(c2=O)-c2ccccc2COCc2cccc-3c2)CCN1C(=O)c1cc2cc(-c3ccnc(C4CCC4)c3)ccc2n1[C@@]1(c2noc(=O)[nH]2)C[C@@H]1C] 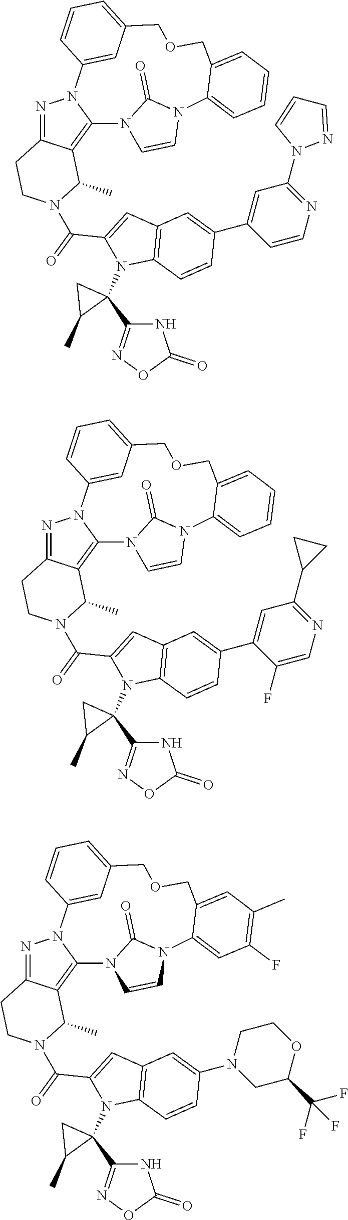 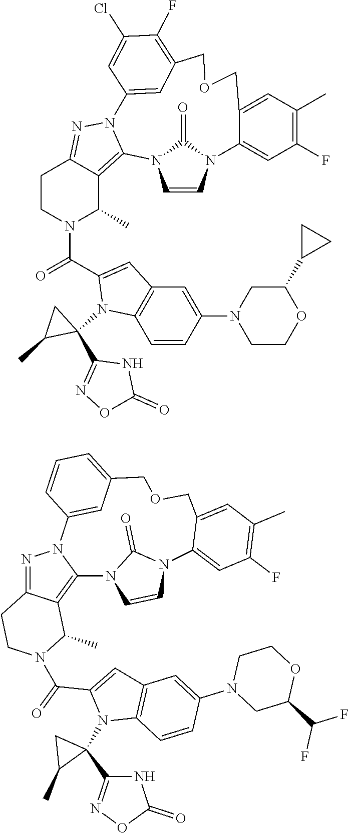 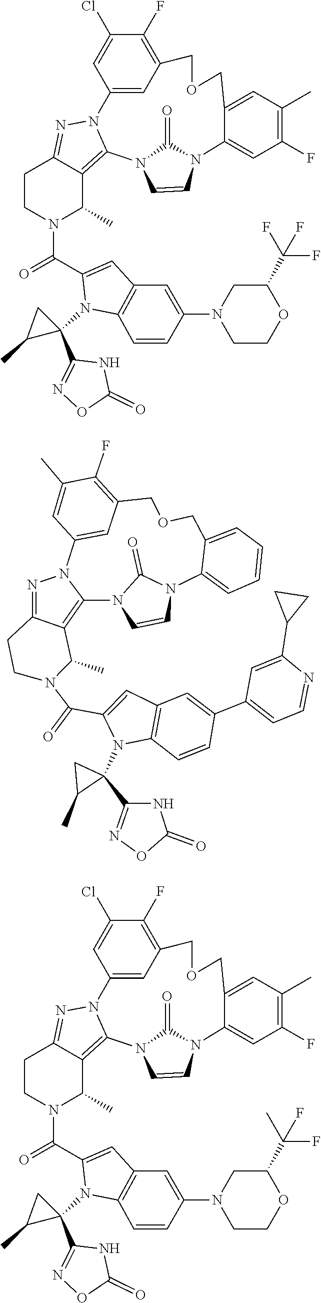 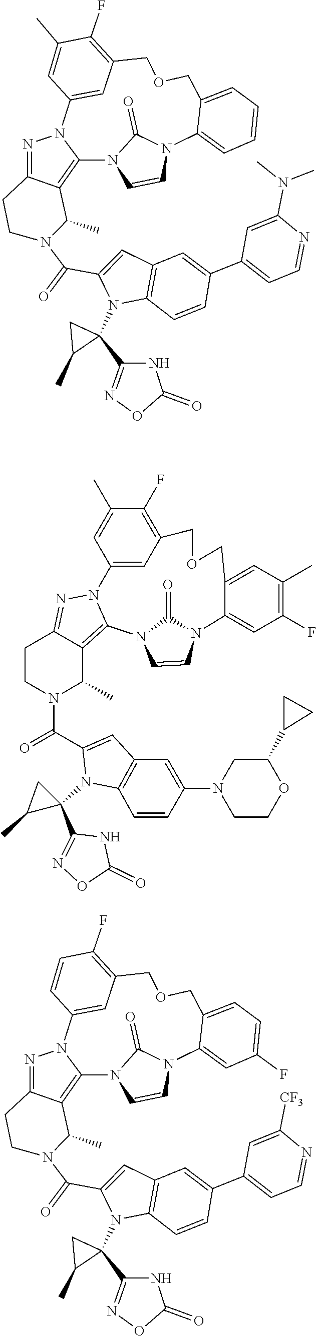 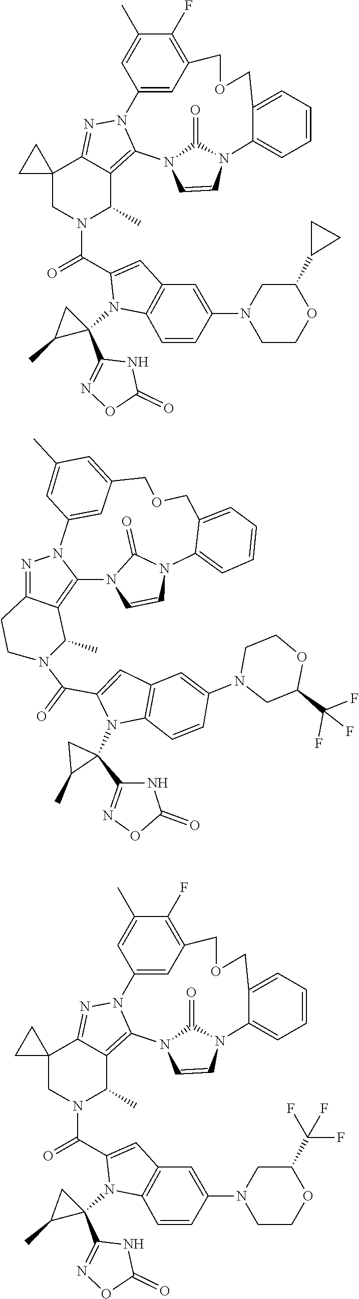 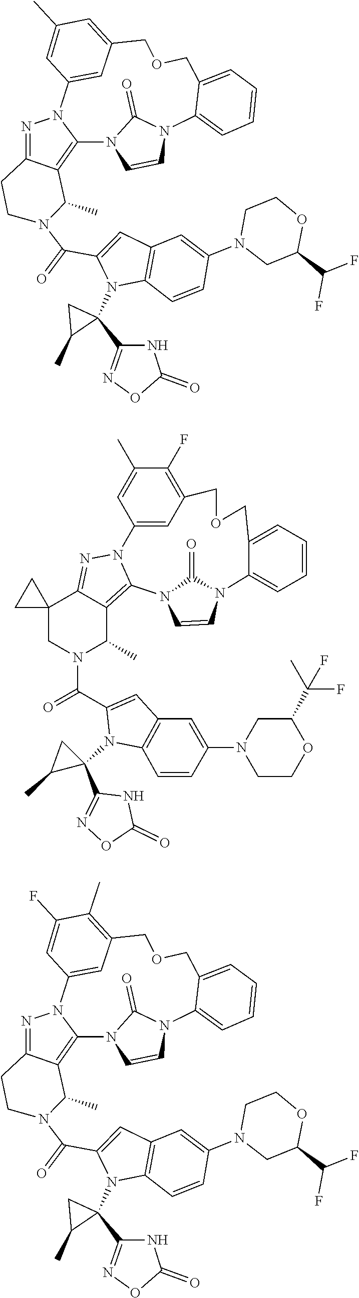 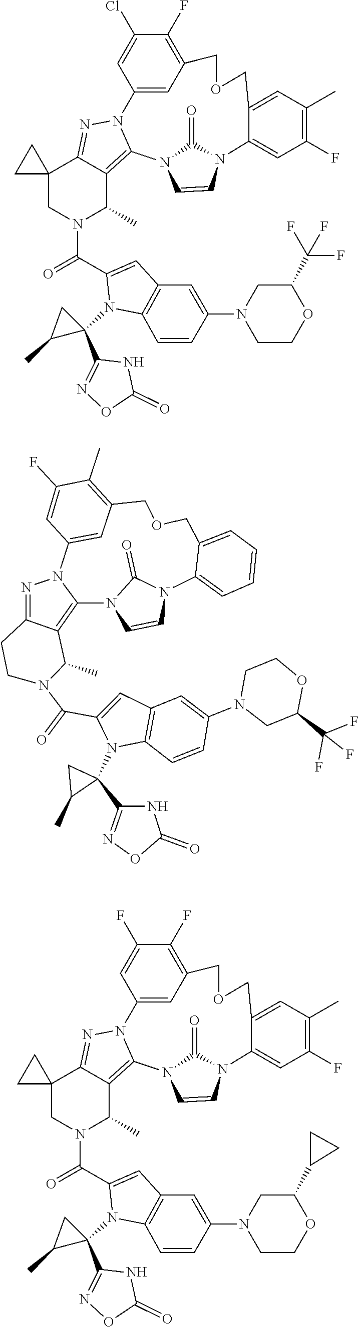 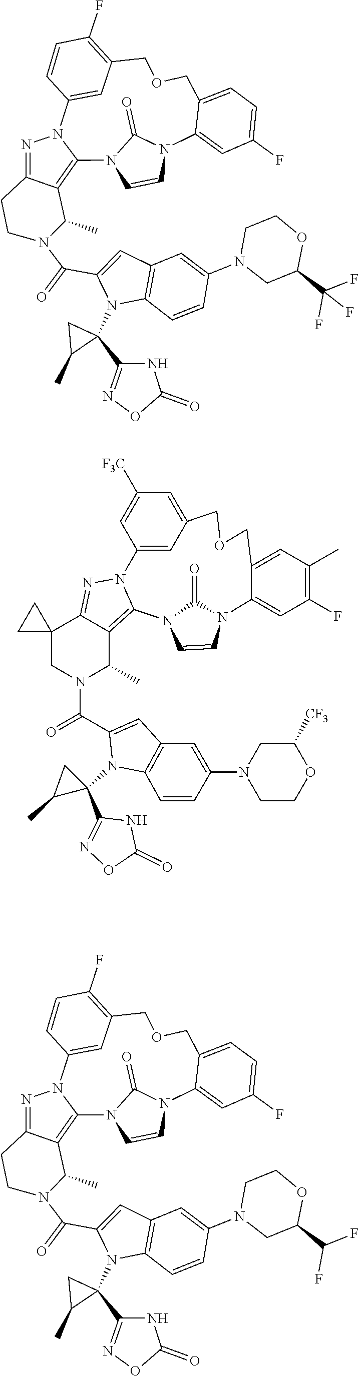 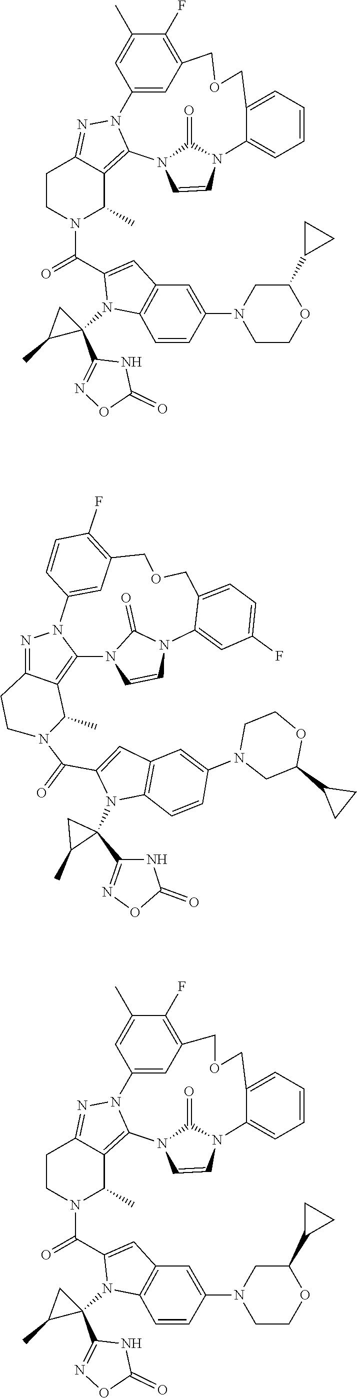 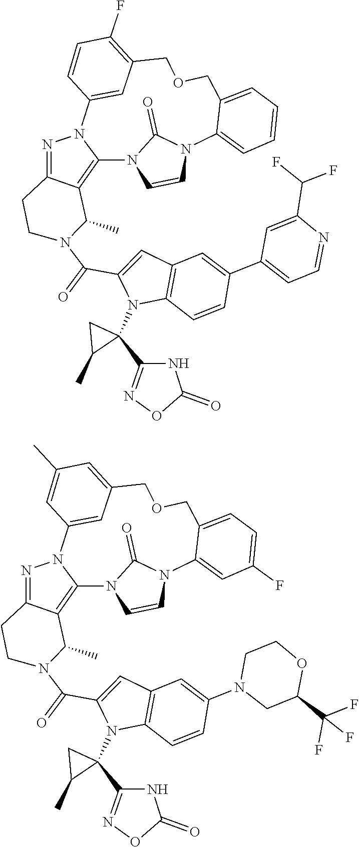 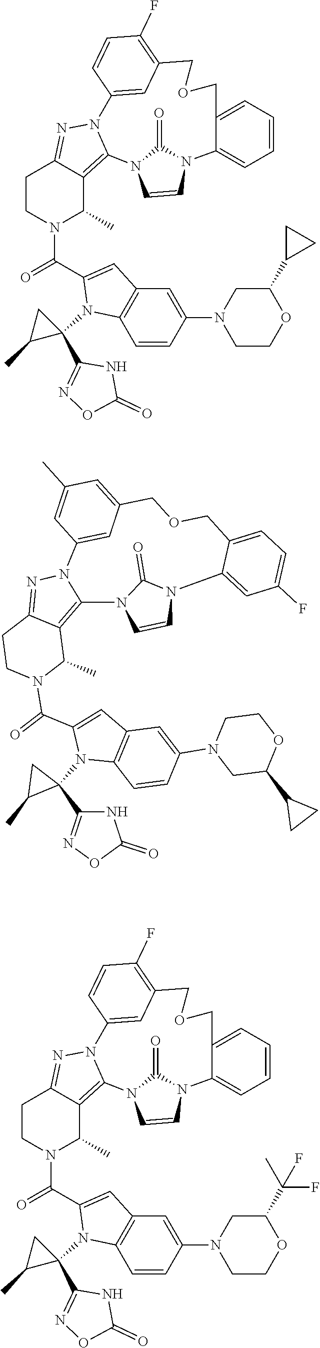 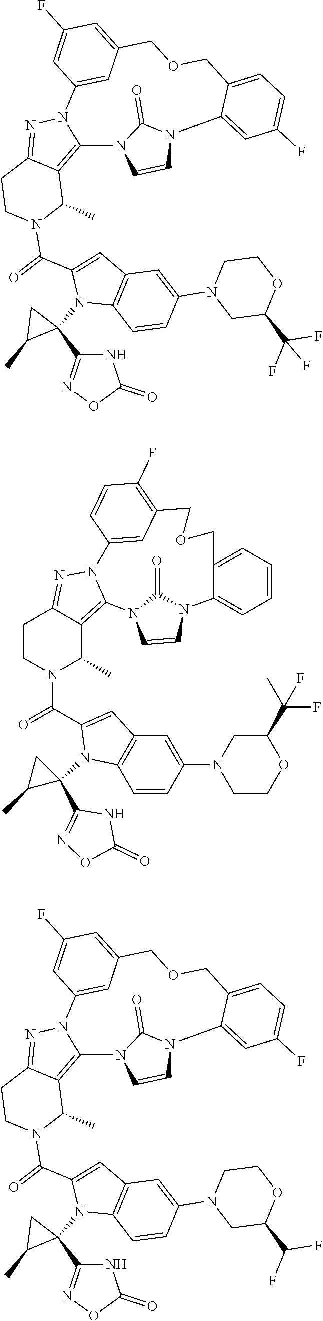 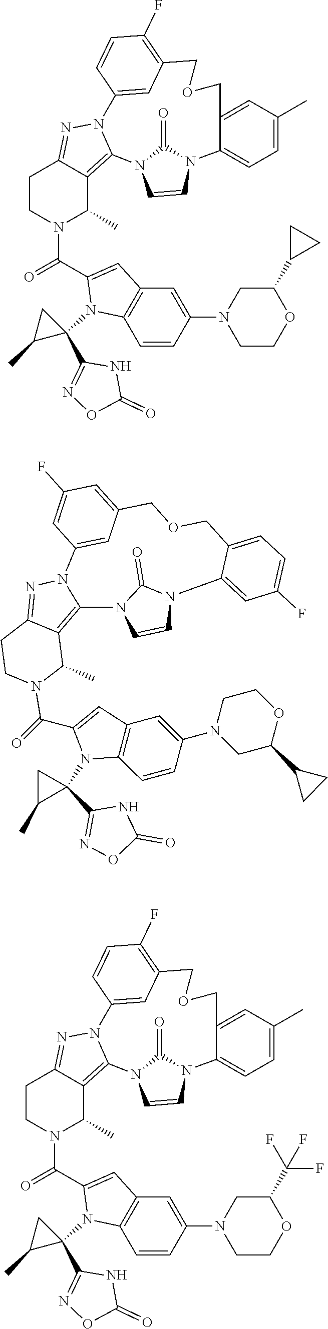 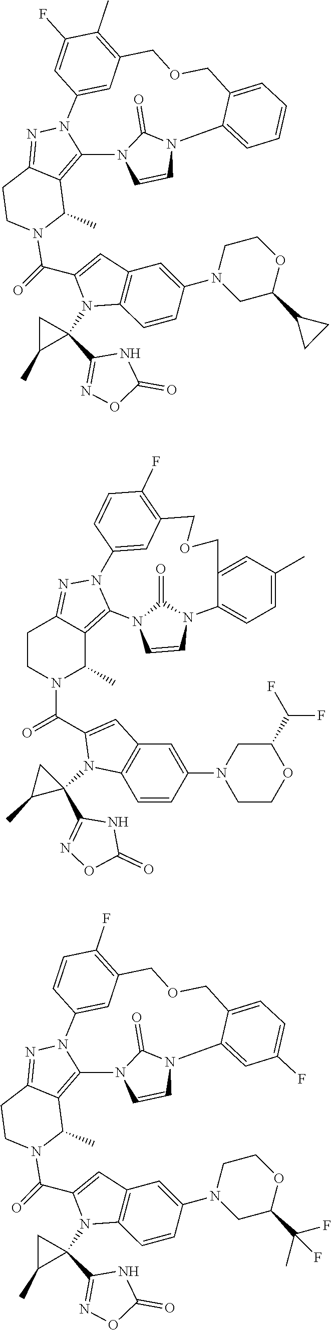 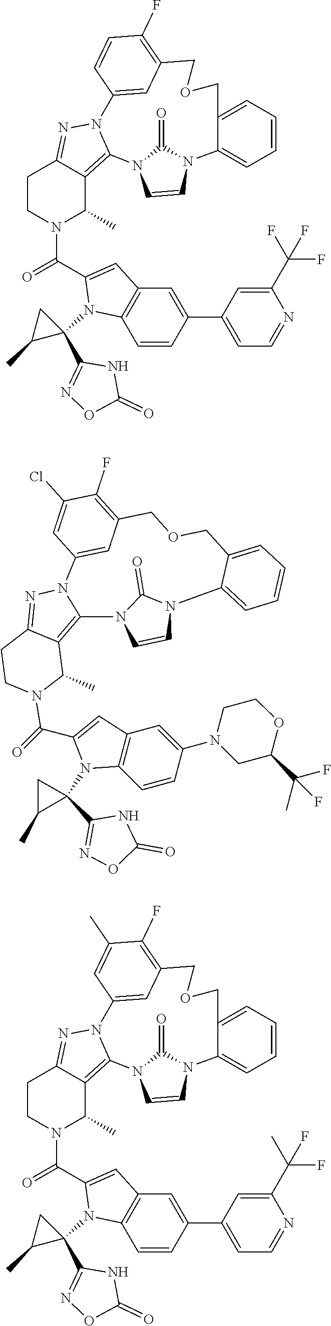 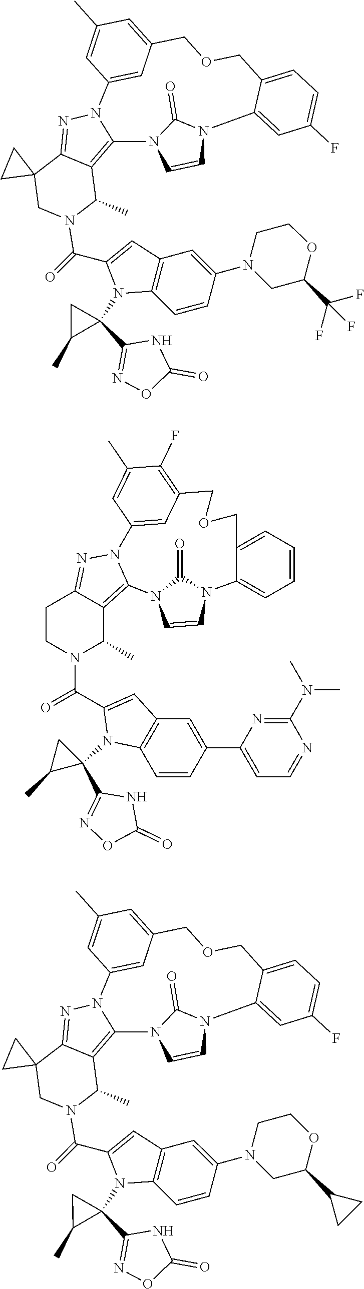 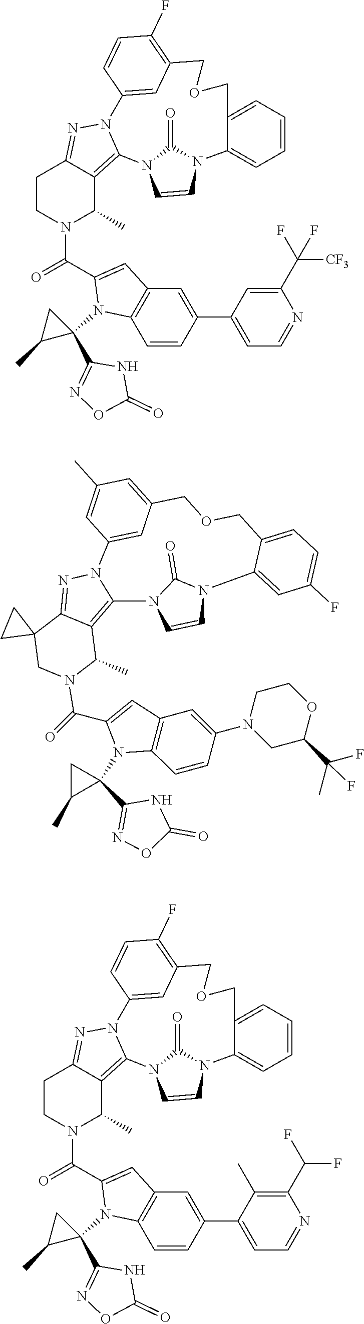 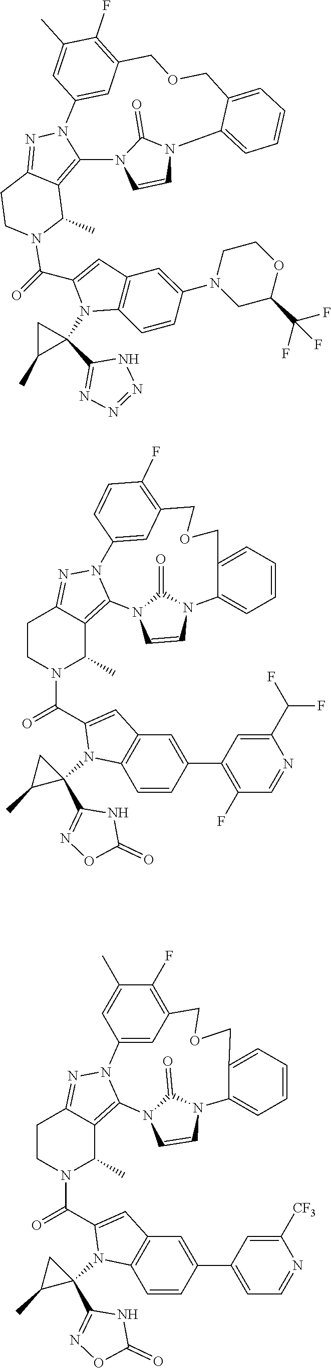 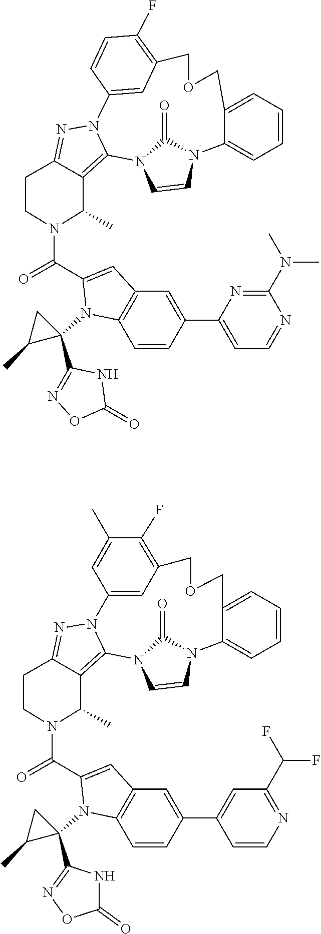 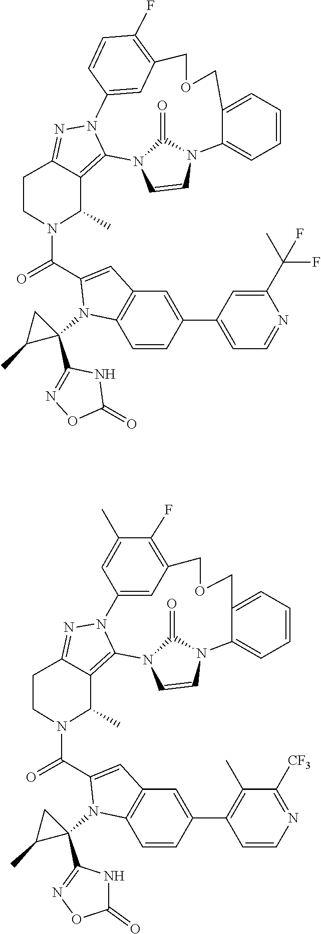 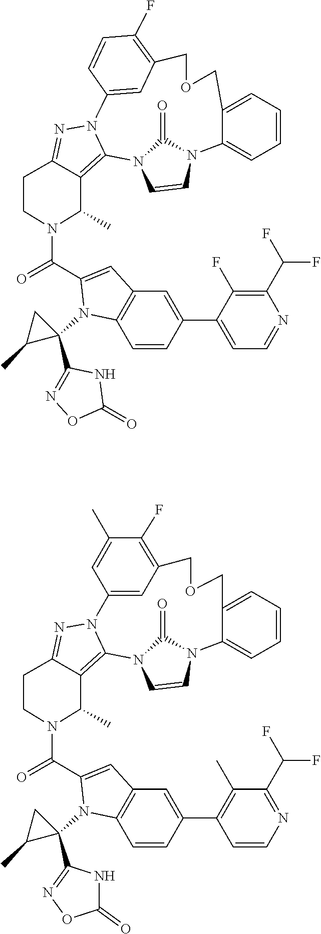 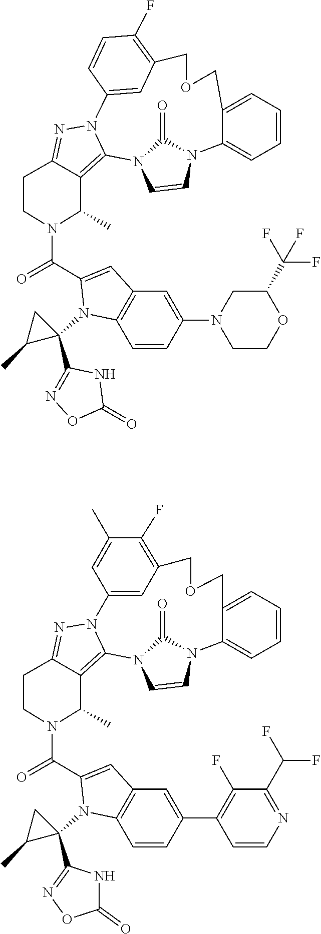 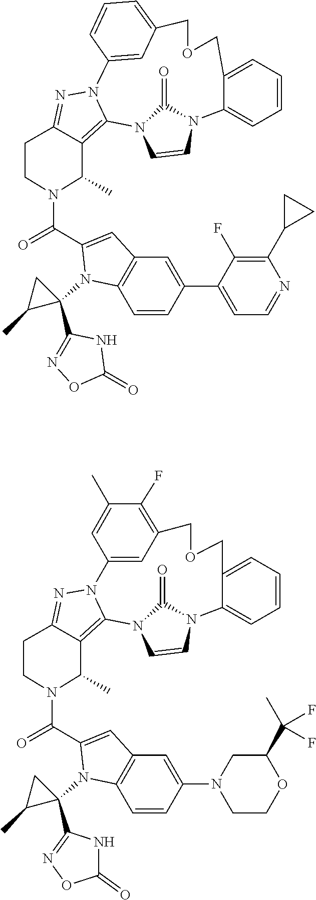 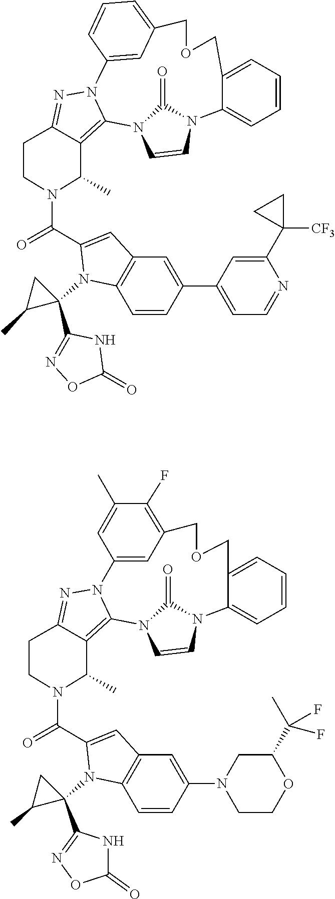 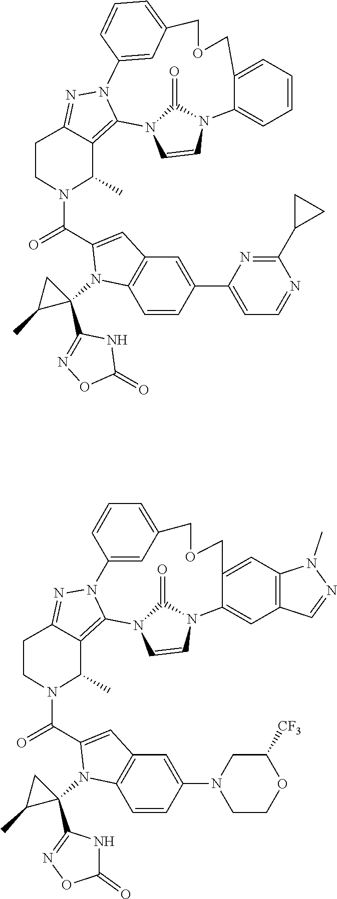 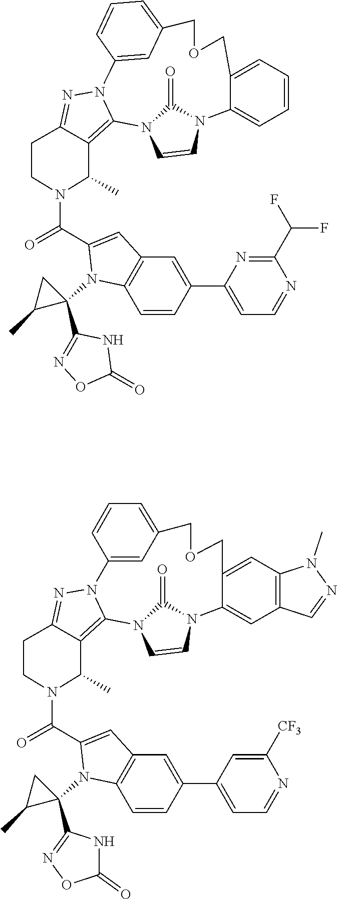 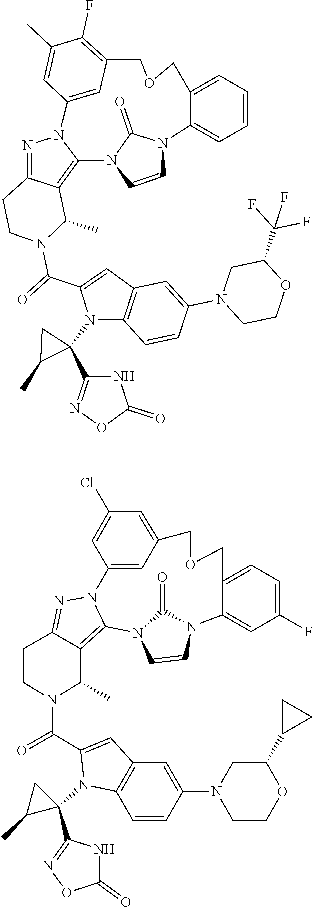 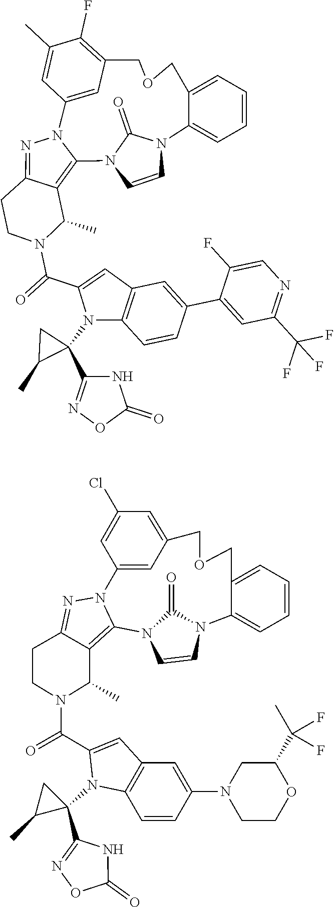 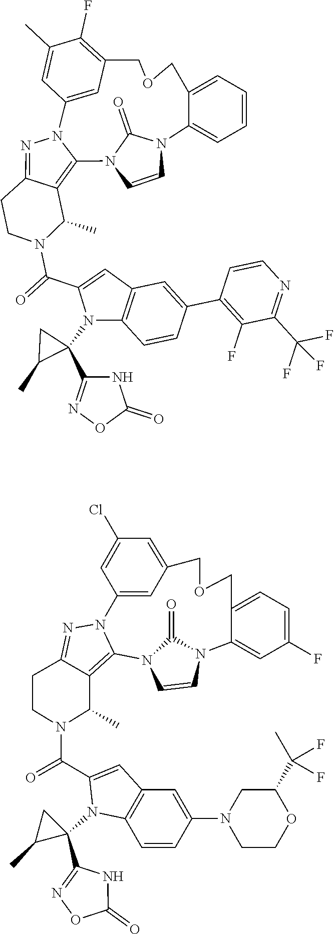 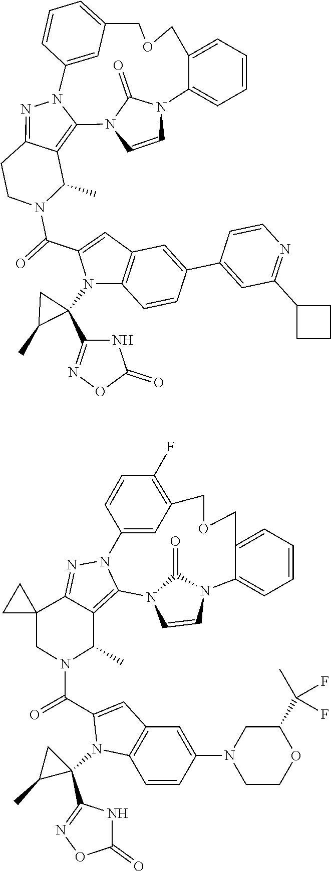 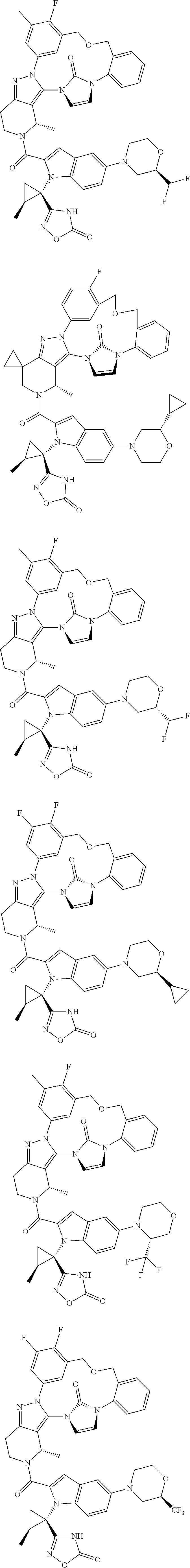 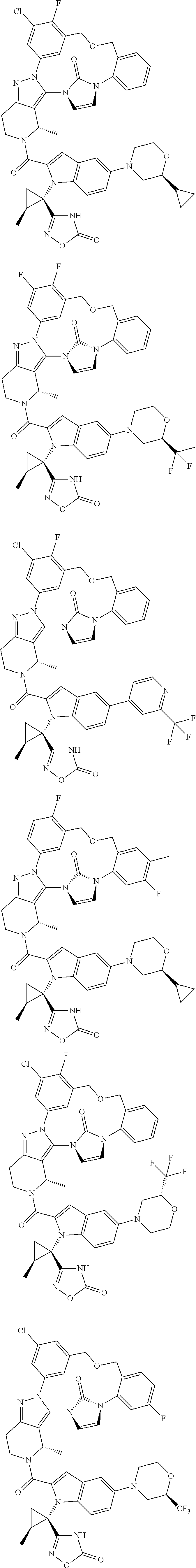 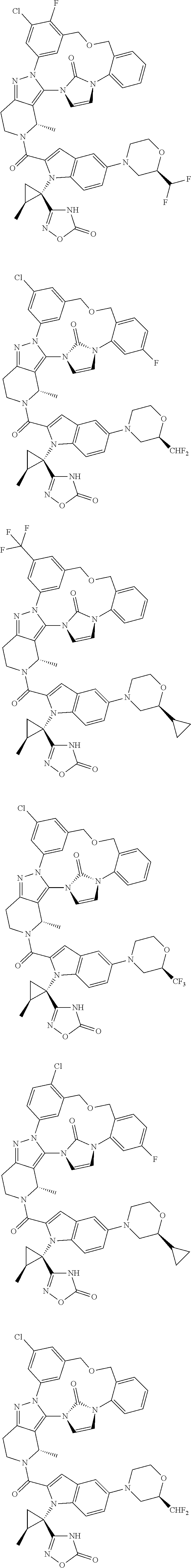 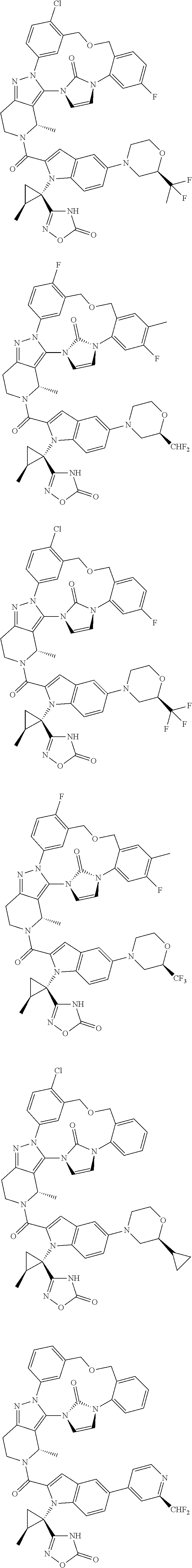 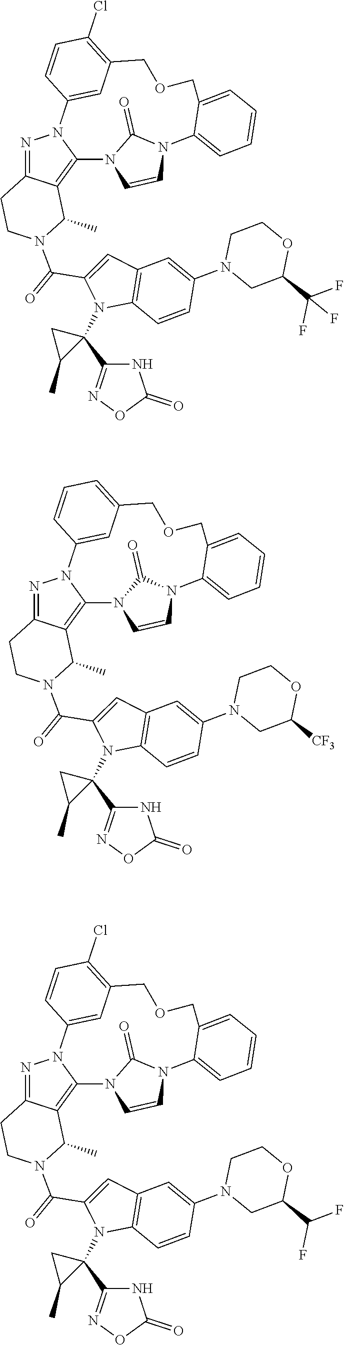 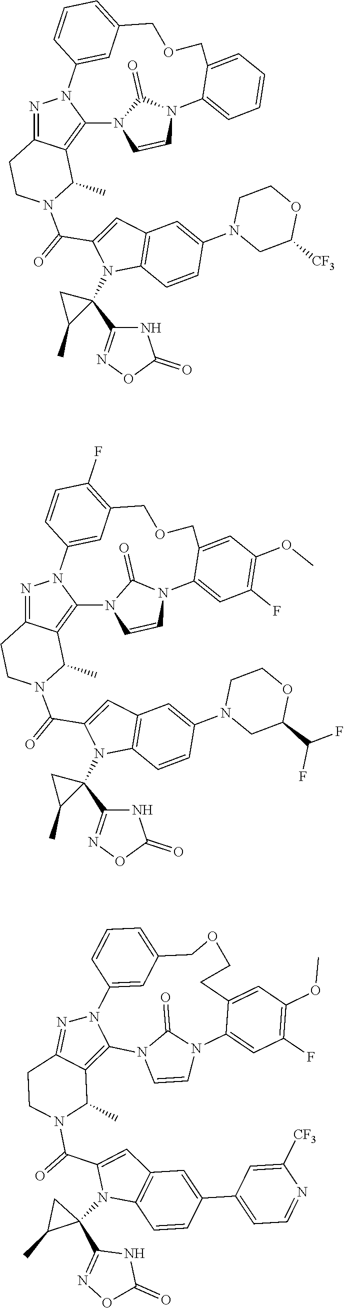 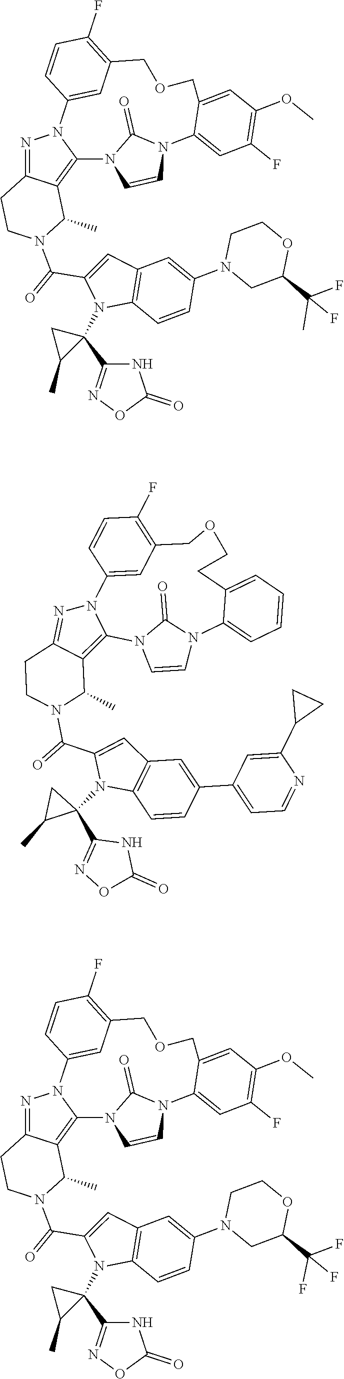 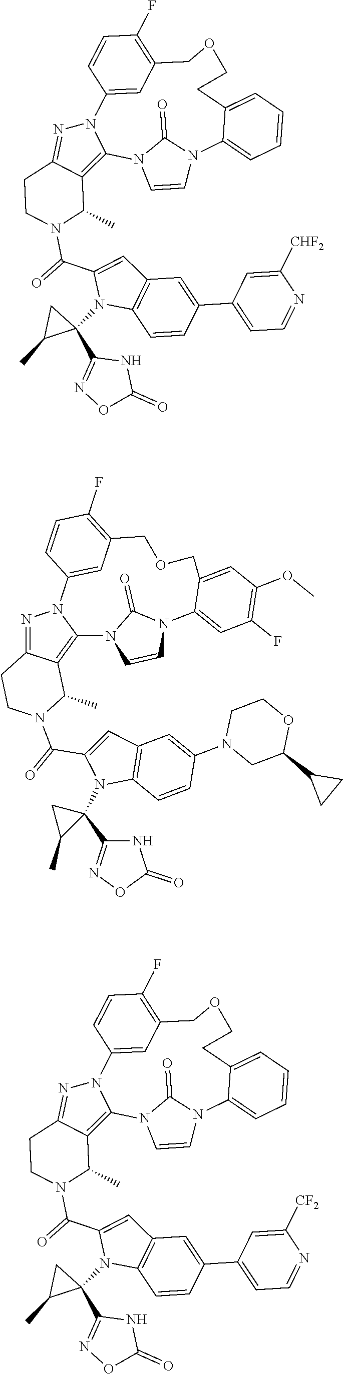 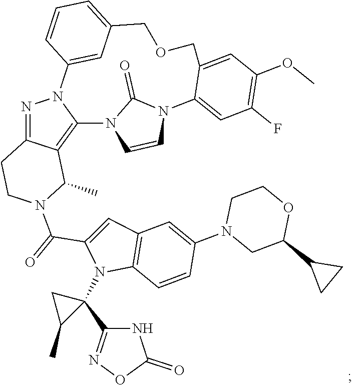
or a pharmaceutically acceptable salt thereof.

61. (canceled)

62. A compound, wherein the compound is selected from: 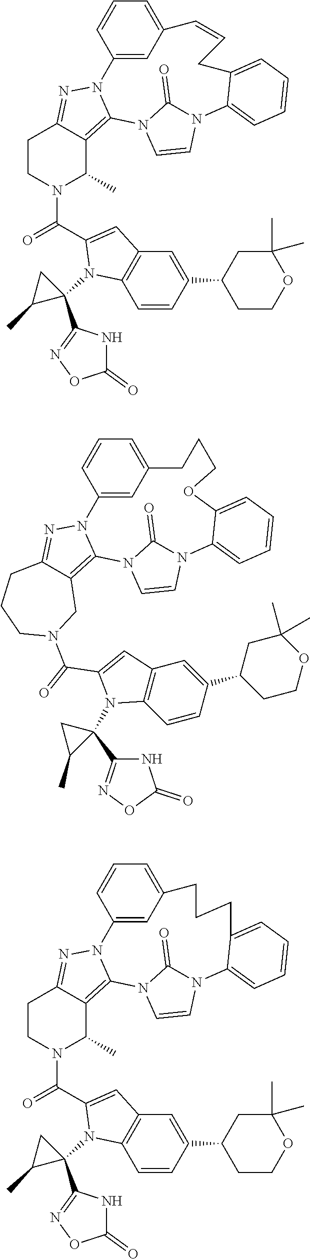 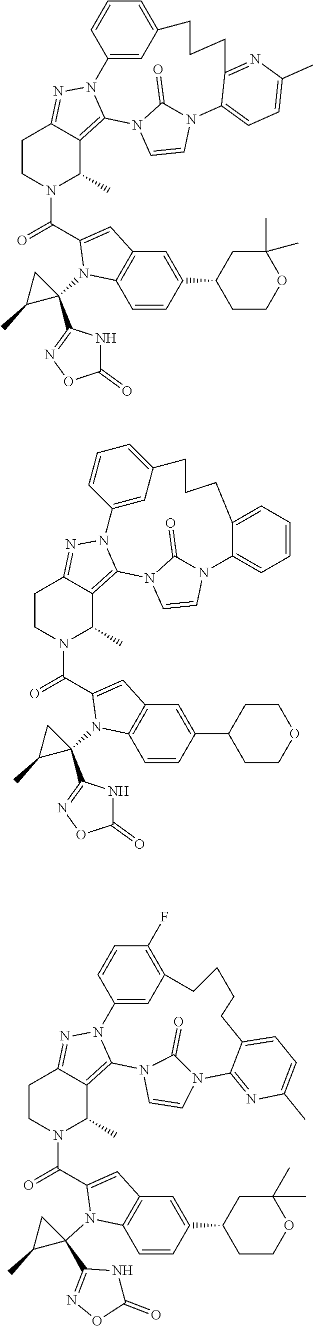 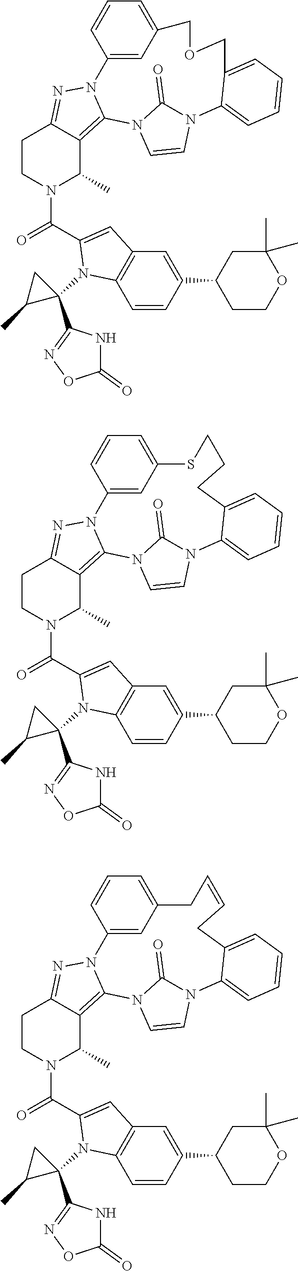 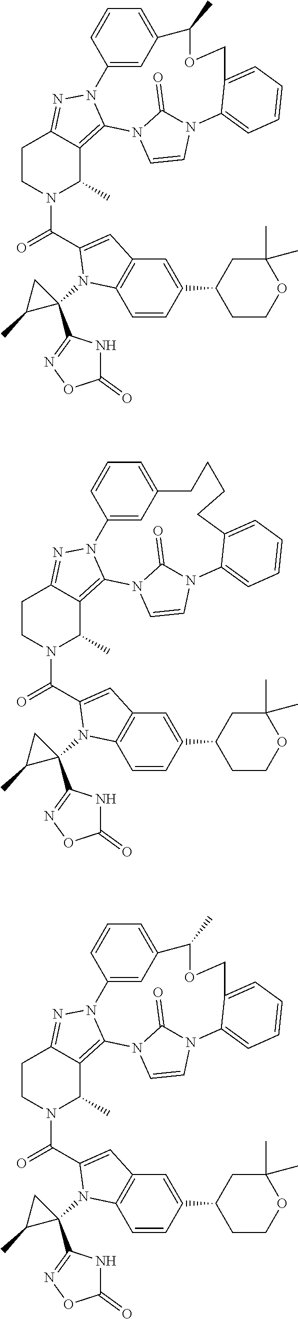 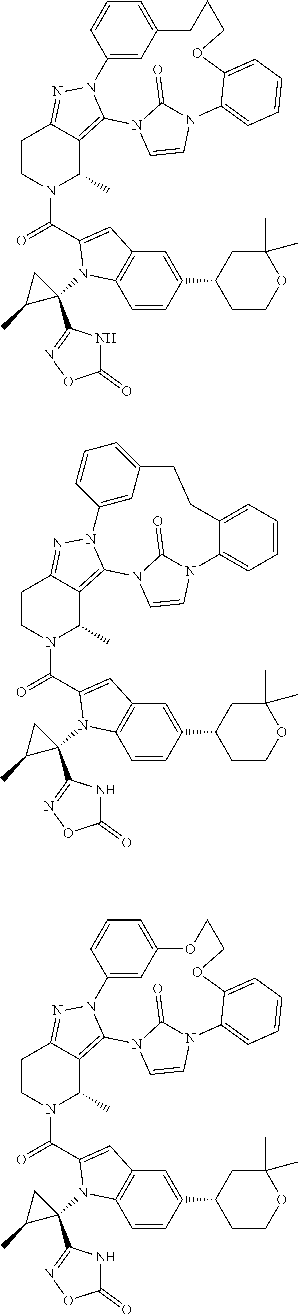 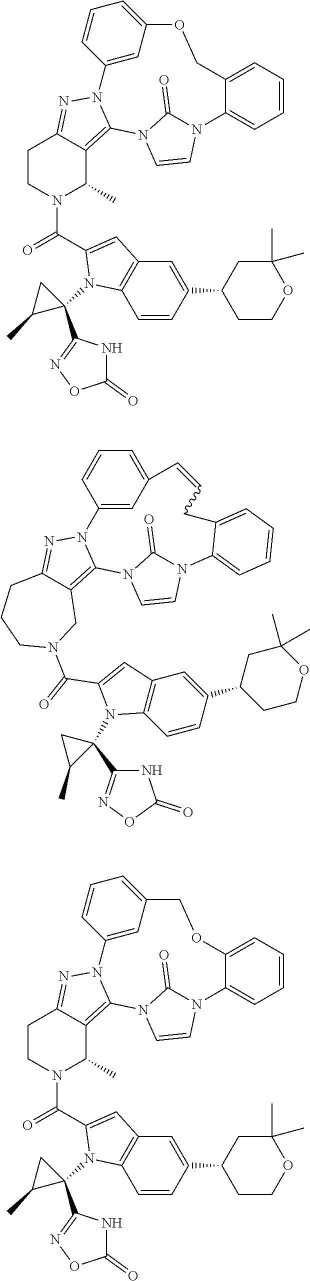 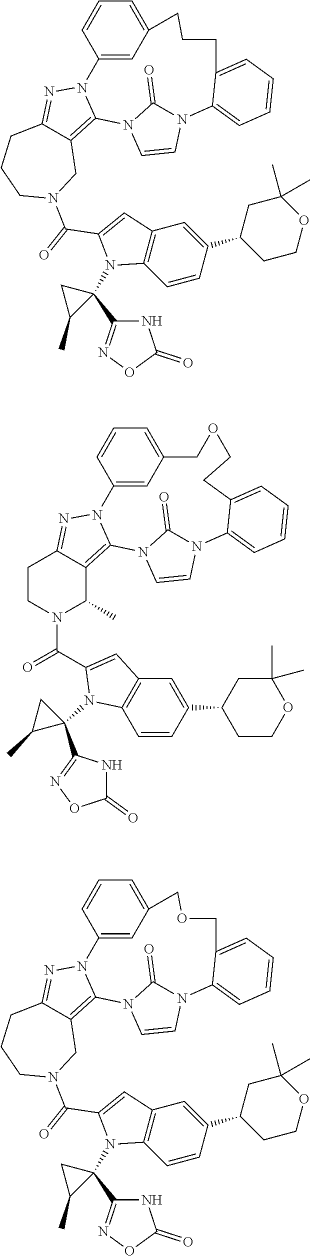 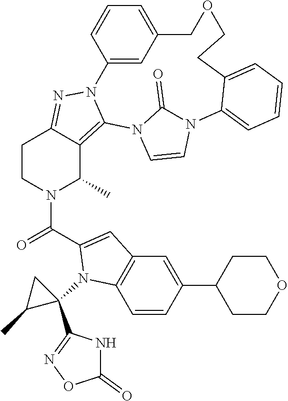 [SMILES: C[C@H]1c2c(nn3c2-n2ccn(c2=O)-c2ccccc2CCOCc2cccc-3c2)CCN1C(=O)c1cc2cc(C3CCOCC3)ccc2n1[C@@]1(c2noc(=O)[nH]2)C[C@@H]1C] 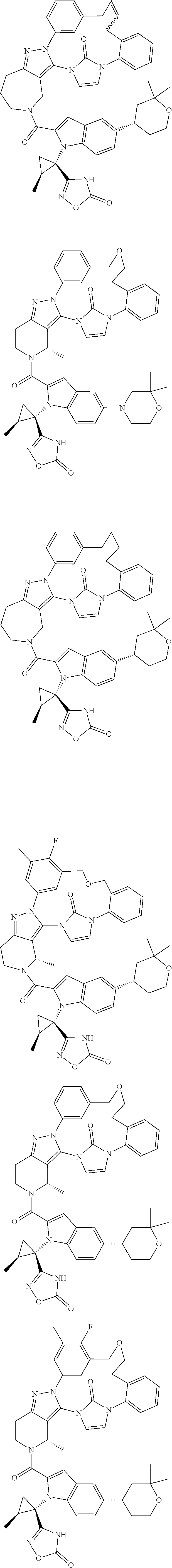 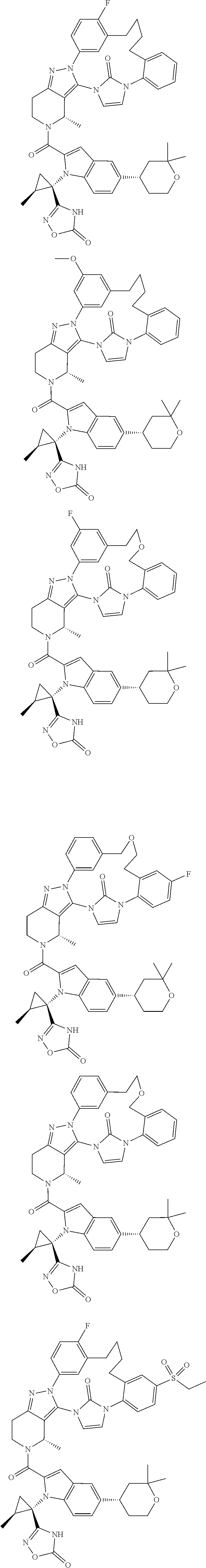 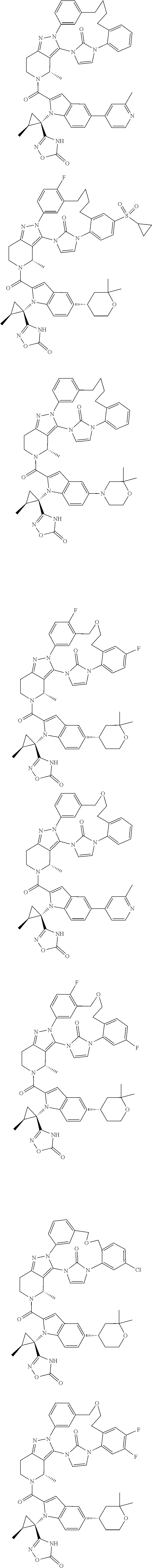 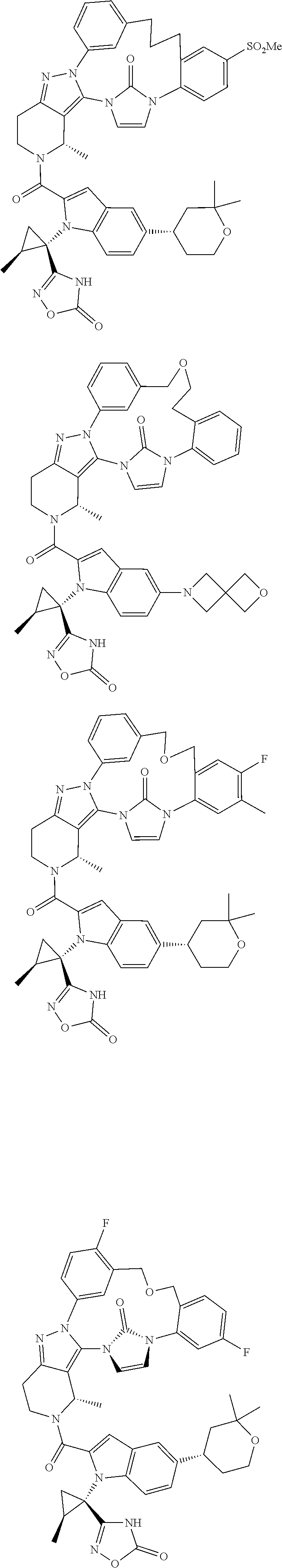 [chemical structure] 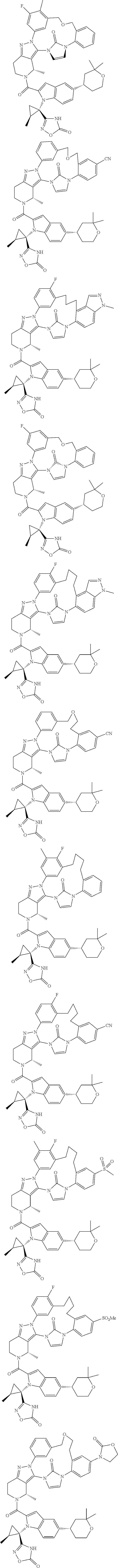 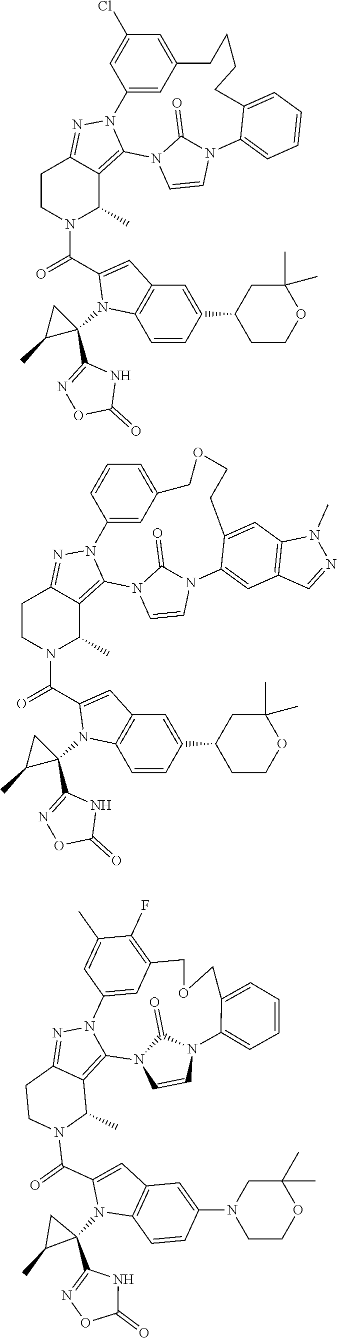 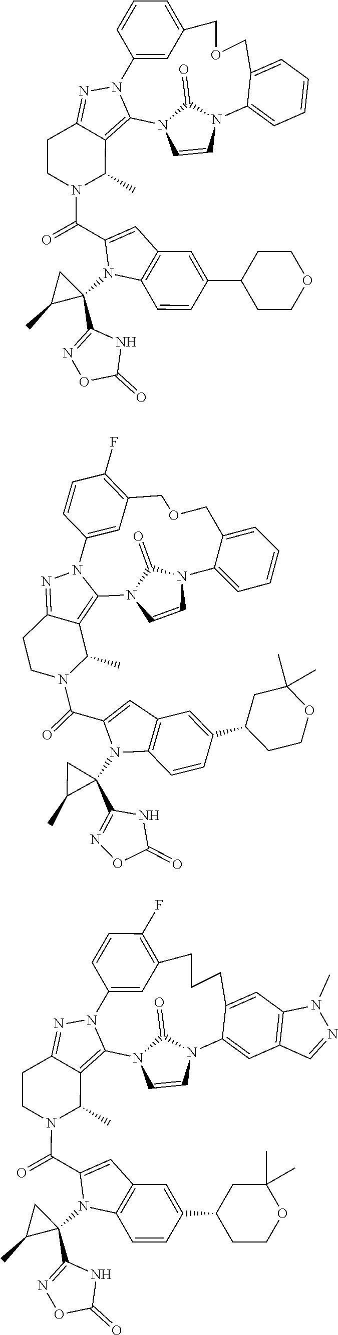 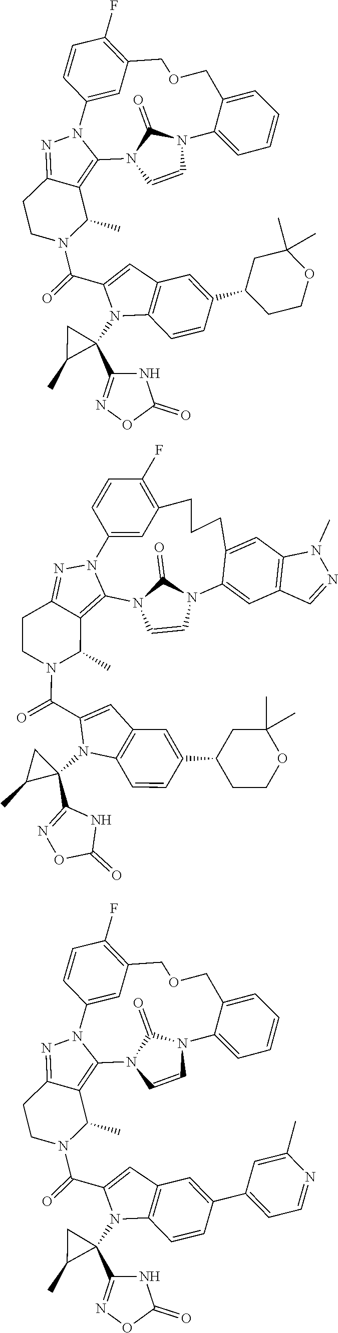 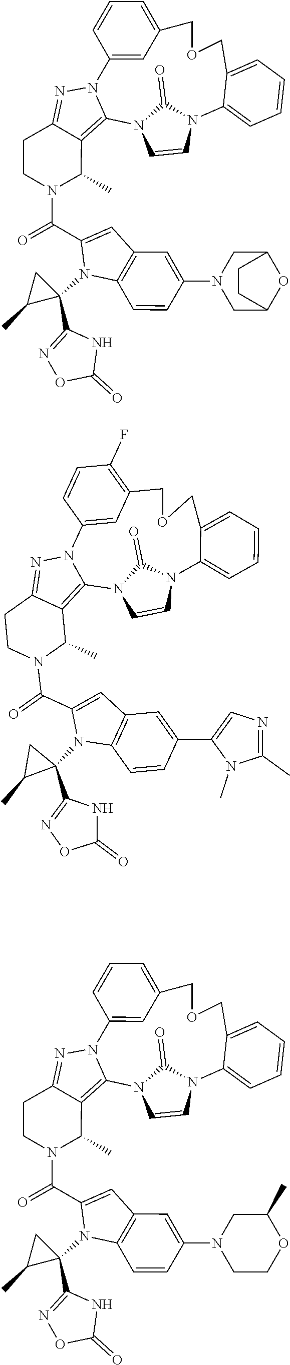 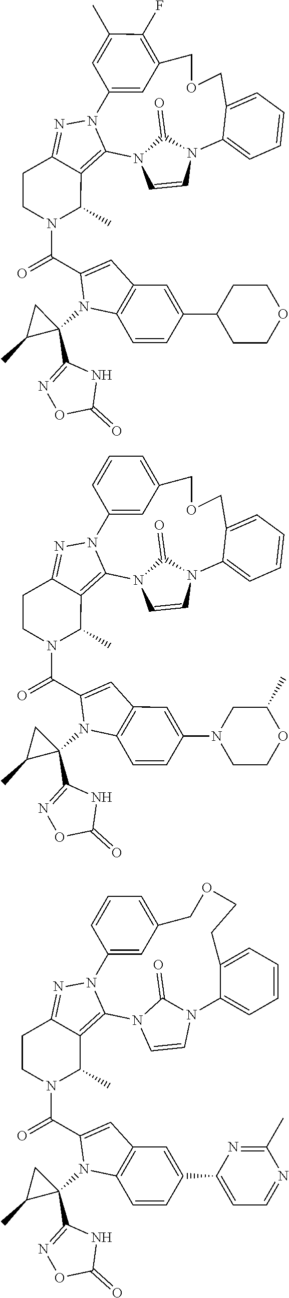 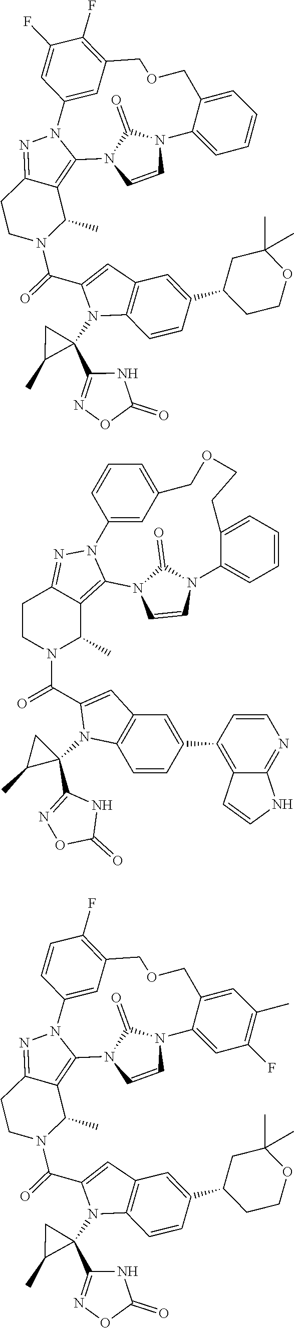 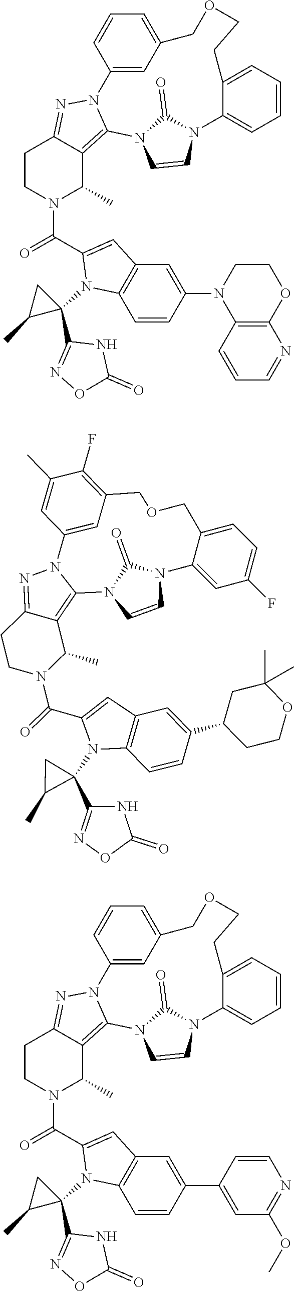 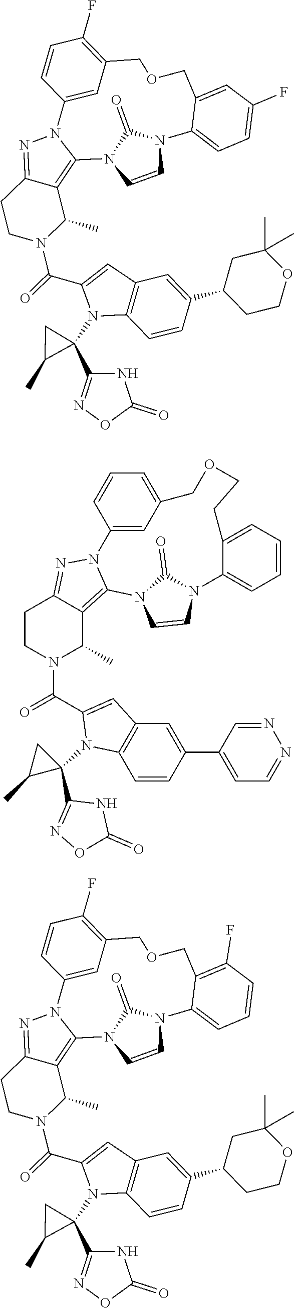 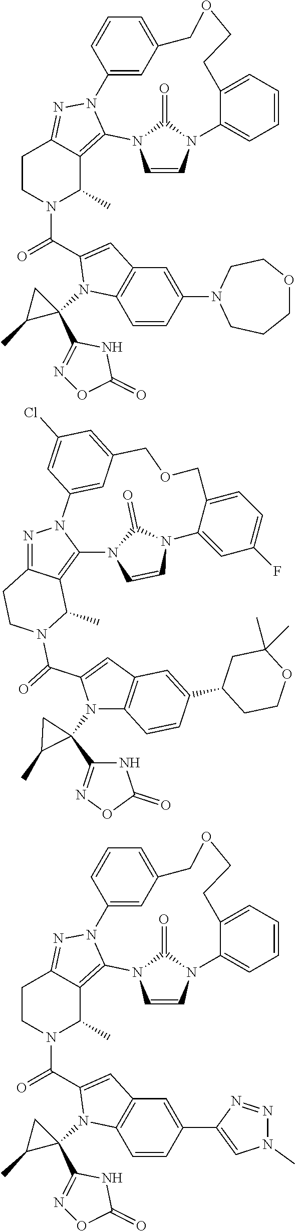 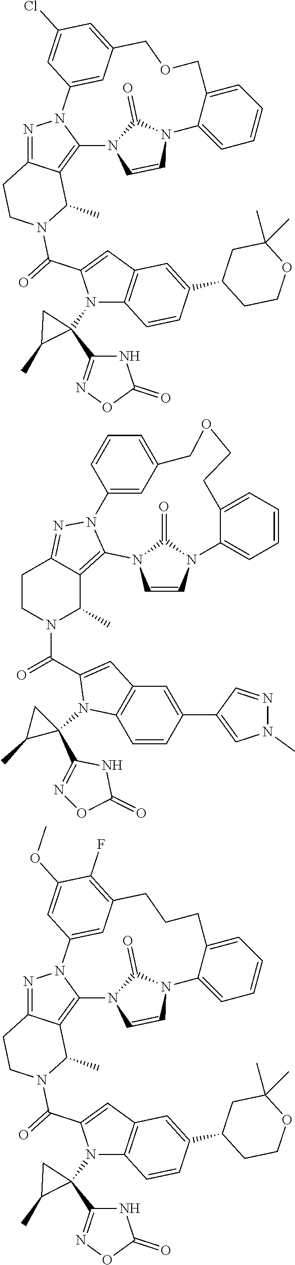 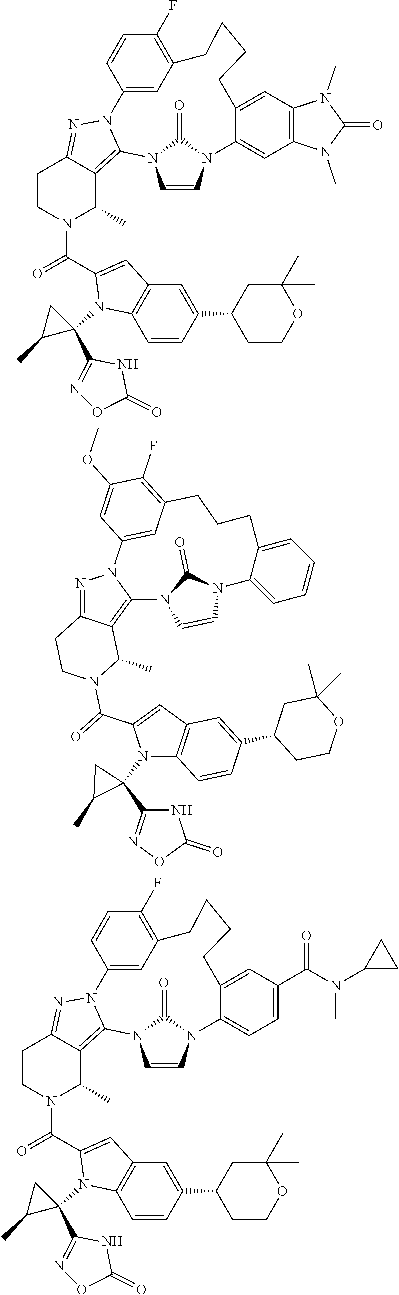 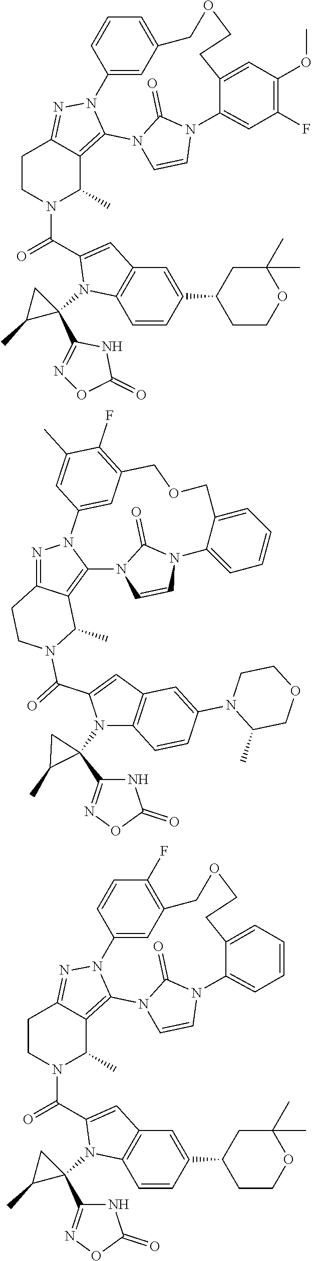 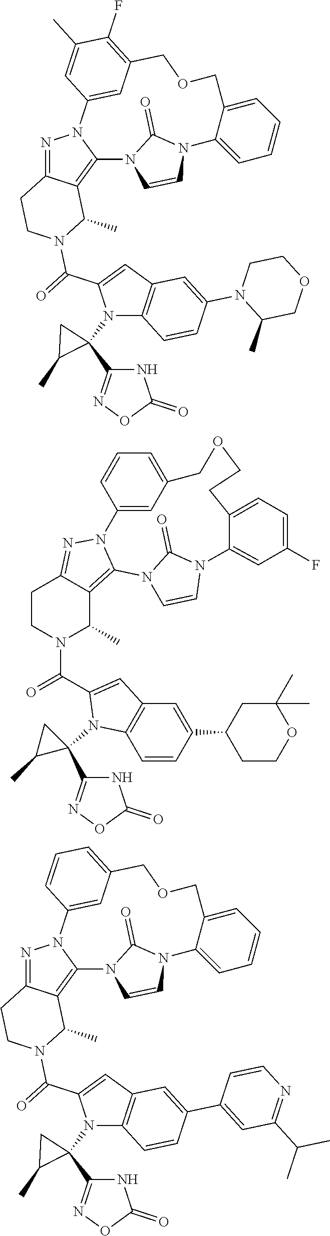 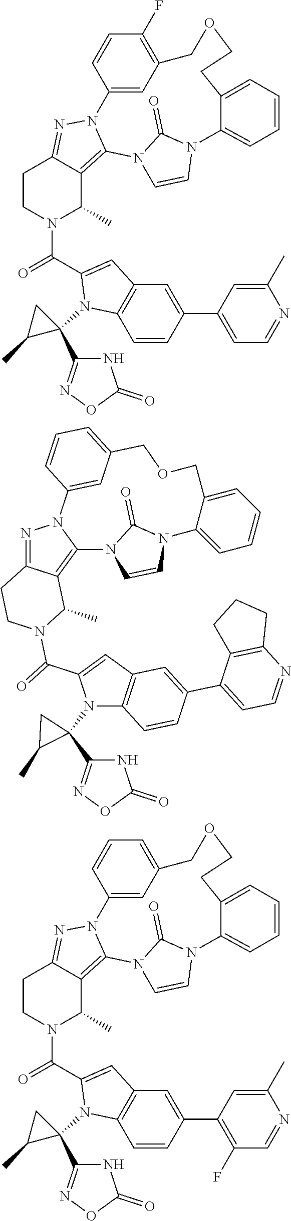 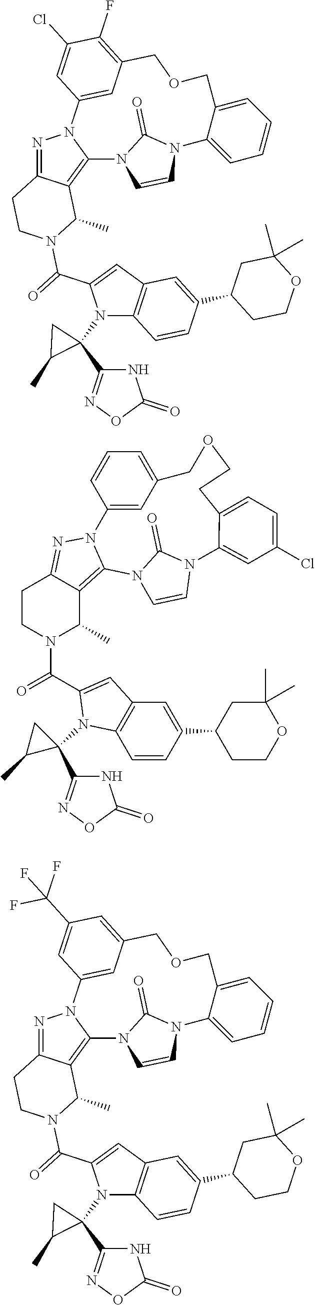 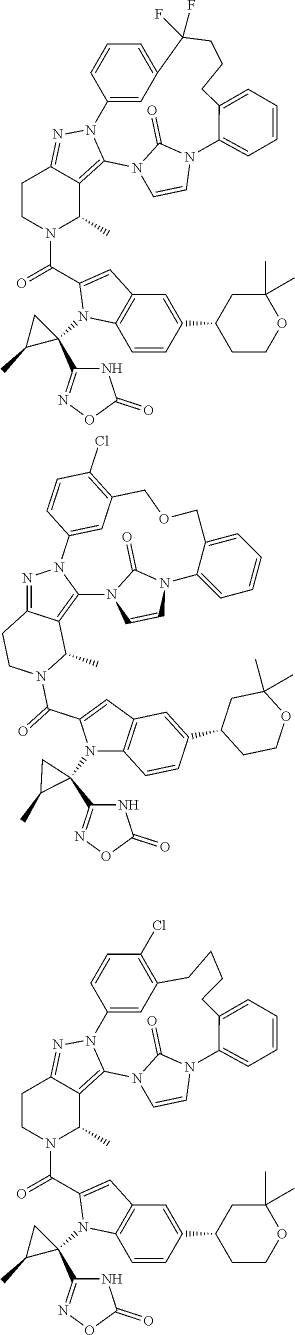 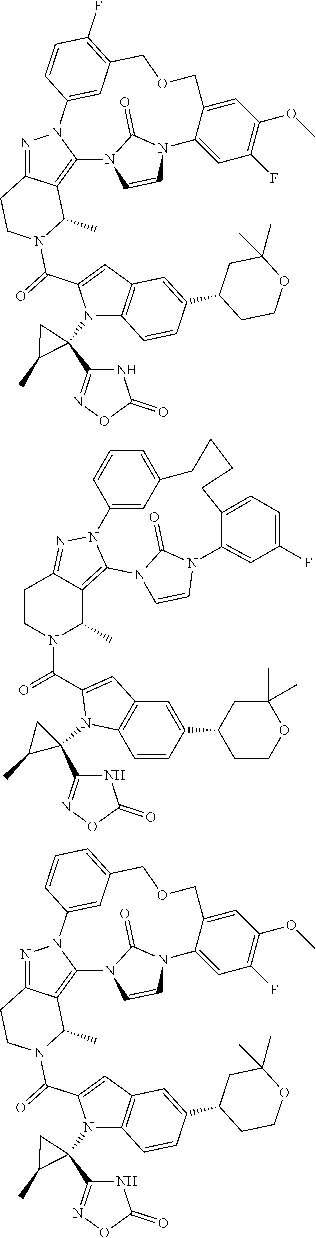 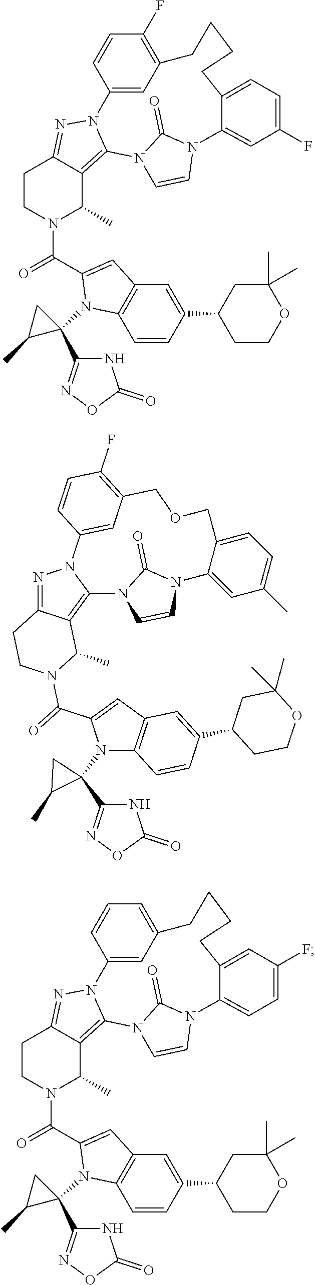
or a pharmaceutically acceptable salt thereof.

63. (canceled)

64. A pharmaceutical composition comprising a compound of claim 1, or a pharmaceutically acceptable salt thereof, and a pharmaceutically acceptable excipient.

65. (canceled)

66. A method of treating a GLP-1-associated disease, disorder, or condition in a subject in need thereof, the method comprising administering to the subject a therapeutically effective amount of a compound of claim 1, or a pharmaceutically acceptable salt thereof.

67-70. (canceled)
